(19) United States
(12) Patent Application Publication
(10) Pub. No.: US 2009/0018134 A1
(43) Pub. Date: Jan. 15, 2009

(54) Compounds - 945

(75) Inventors: Kurt Gordon PIKE, Macclesfield (GB); Jeffrey James Morris, Macclesfield (GB)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/170,128

(22) Filed: Jul. 9, 2008

Related U.S. Application Data
(60) Provisional application No. 60/948,566, filed on Jul. 9, 2007.
(60) Provisional application No. 61/030,297, filed on Feb. 21, 2008.

(51) Int. Cl. A61K 31/5377; C07D 413/04; A61P 35/04; C07D 413/14

(52) U.S. Cl. 514/235.8; 544/122

(57) ABSTRACT

A compound of formula (I)

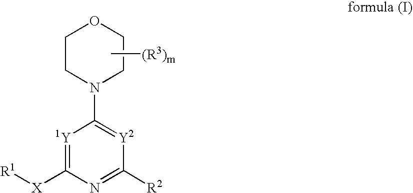

or a pharamaceutically acceptable salt thereof, processes for their preparation, pharmaceutical compositions containing them and their use in therapy, for example in the treatment of proliferative disease such as cancer and particularly in disease mediated by an mTOR kinase and/or one or more PI3K enzyme.

This application claims the benefit under under 35 U.S.C. §119(e) of Application No U.S. 60/948566 filed on 9 Jul. 2007 and of Application U.S. No. 61/030297 filed on 21 Feb. 2008.

The present invention relates to morpholino pyrimidine compounds, processes for their preparation, pharmaceutical compositions containing them and their use in therapy, for example in the treatment of proliferative disease such as cancer and particularly in disease mediated by an mTOR kinase and/or one or more PI3K enzyme.

It is now well understood that deregulation of oncogenes and tumour-suppressor genes contributes to the formation of malignant tumours, for example by way of increased cell proliferation or increased cell survival. It is also known that signalling pathways mediated by the PI3K/mTOR families have a central role in a number of cell processes including proliferation and survival, and deregulation of these pathways is a causative factor in a wide spectrum of human cancers and other diseases.

The mammalian target of the macrolide antibiotic Rapamycin (sirolimus) is the enzyme mTOR. This enzymes belongs to the phosphatidylinositol (PI) kinase-related kinase (PIKK) family of protein kinases, which also includes ATM, ATR, DNA-PK and hSMG-1. mTOR, like other PIKK family members, does not possess detectable lipid kinase activity, but instead functions as a serine/threonine kinase. Much of the knowledge of mTOR signalling is based upon the use of Rapamycin. Rapamycin first binds to the 12 kDa immunophilin FK506-binding protein (FKBP12) and this complex inhibits mTOR signalling (Tee and Blenis, Seminars in Cell and Developmental Biology, 2005, 16, 29-37). The mTOR protein consists of a catalytic kinase domain, an FKBP12-Rapamycin binding (FRB) domain, a putative repressor domain near the C-terminus and up to 20 tandemly-repeated HEAT motifs at the N-terminus, as well as FRAP-ATM-TRRAP (FAT) and FAT C-terminus domain (Huang and Houghton, Current Opinion in Pharmacology, 2003, 3, 371-377).

mTOR kinase is a key regulator of cell growth and has been shown to regulate a wide range of cellular functions including translation, transcription, mRNA turnover, protein stability, actin cytoskeleton reorganisation and autophagy (Jacinto and Hall, Nature Reviews Molecular and Cell Biology, 2005, 4, 117-126). mTOR kinase integrates signals from growth factors (such as insulin or insulin-like growth factor) and nutrients (such as amino acids and glucose) to regulate cell growth. mTOR kinase is activated by growth factors through the PI3K-Akt pathway. The most well characterised function of mTOR kinase in mammalian cells is regulation of translation through two pathways, namely activation of ribosomal S6K1 to enhance translation of mRNAs that bear a 5′-terminal oligopyrimidine tract (TOP) and suppression of 4E-BP1 to allow CAP-dependent mRNA translation.

Generally, investigators have explored the physiological and pathological roles of mTOR using inhibition with Rapamycin and related Rapamycin analogues based on their specificity for mTOR as an intracellular target. However, recent data suggests that Rapamycin displays variable inhibitory actions on mTOR signalling functions and suggest that direct inhibition of the mTOR kinase domain may display substantially broader anti-cancer activities than that achieved by Rapamycin (Edinger et al., Cancer Research, 2003, 63, 8451-8460). For this reason, potent and selective inhibitors of mTOR kinase activity would be useful to allow a more complete understanding of mTOR kinase function and to provide useful therapeutic agents.

There is now considerable evidence indicating that the pathways upstream of mTOR, such as the PI3K pathway, are frequently activated in cancer (Vivanco and Sawyers, Nature Reviews Cancer, 2002, 2, 489-501; Bjornsti and Houghton, Nature Reviews Cancer, 2004, 4, 335-348; Inoki et al., Nature Genetics, 2005, 37, 19-24). For example, components of the PI3K pathway that are mutated in different human tumours include activating mutations of growth factor receptors and the amplification and/or overexpression of PI3K and Akt.

In addition there is evidence that endothelial cell proliferation may also be dependent upon mTOR signalling. Endothelial cell proliferation is stimulated by vascular endothelial cell growth factor (VEGF) activation of the PI3K-Akt-mTOR signalling pathway (Dancey, Expert Opinion on Investigational Drugs, 2005, 14, 313-328). Moreover, mTOR kinase signalling is believed to partially control VEGF synthesis through effects on the expression of hypoxia-inducible factor-1α (HIF-1α) (Hudson et al., Molecular and Cellular Biology, 2002, 22, 7004-7014). Therefore, tumour angiogenesis may depend on mTOR kinase signalling in two ways, through hypoxia-induced synthesis of VEGF by tumour and stromal cells, and through VEGF stimulation of endothelial proliferation and survival through PI3K-Akt-mTOR signalling.

These findings suggest that pharmacological inhibitors of mTOR kinase should be of therapeutic value for treatment of the various forms of cancer comprising solid tumours such as carcinomas and sarcomas and the leukaemias and lymphoid malignancies. In particular, inhibitors of mTOR kinase should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

In addition to tumourigenesis, there is evidence that mTOR kinase plays a role in an array of hamartoma syndromes. Recent studies have shown that the tumour suppressor proteins such as TSC1, TSC2, PTEN and LKB1 tightly control mTOR kinase signalling. Loss of these tumour suppressor proteins leads to a range of hamartoma conditions as a result of elevated mTOR kinase signalling (Tee and Blenis, Seminars in Cell and Developmental Biology, 2005, 16, 29-37). Syndromes with an established molecular link to dysregulation of mTOR kinase include Peutz-Jeghers syndrome (PJS), Cowden disease, Bannayan-Riley-Ruvalcaba syndrome (BRRS), Proteus syndrome, Lhermitte-Duclos disease and Tuberous Sclerosis (TSC) (Inoki et al., Nature Genetics, 2005, 37, 19-24). Patients with these syndromes characteristically develop benign hamartomatous tumours in multiple organs.

Recent studies have revealed a role for mTOR kinase in other diseases (Easton & Houghton, Expert Opinion on Therapeutic Targets, 2004, 8, 551-564). Rapamycin has been demonstrated to be a potent immunosuppressant by inhibiting antigen-induced proliferation of T cells, B cells and antibody production (Sehgal, Transplantation Proceedings, 2003, 35, 7S-14S) and thus mTOR kinase inhibitors may also be useful immunosuppressives. Inhibition of the kinase activity of mTOR may also be useful in the prevention of restenosis, that is the control of undesired proliferation of normal cells in the vasculature in response to the introduction of stents in the treatment of vasculature disease (Morice et al., New England Journal of Medicine, 2002, 346, 1773-1780). Furthermore, the Rapamycin analogue, everolimus, can reduce the severity and incidence of cardiac allograft vasculopathy (Eisen et al., New England Journal of Medicine, 2003, 349, 847-858). Elevated mTOR kinase activity has been associated with cardiac hypertrophy, which is of clinical importance as a major risk factor for heart failure and is a consequence of increased cellular size of cardiomyocytes (Tee & Blenis, Seminars in Cell and Developmental Biology, 2005, 16, 29-37). Thus mTOR kinase inhibitors are expected to be of value in the prevention and treatment of a wide variety of diseases in addition to cancer.

It is also believed that a number of these morpholino pyrimidine derivatives may have inhibitory activity against the phosphatidylinositol (PI) 3-kinases family of kinases.

Phosphatidylinositol (PI) 3-kinases (PI3Ks) are ubiquitous lipid kinases that function both as signal transducers downstream of cell-surface receptors and in constitutive intracellular membrane and protein trafficking pathways. All PI3Ks are dual-specificity enzymes with a lipid kinase activity that phosphorylates phosphoinositides at the 3-hydroxy position, and a less well characterised protein kinase activity. The lipid products of PI3K-catalysed reactions comprising phosphatidylinositol 3,4,5-trisphosphate[PI(3,4,5)P₃], phosphatidylinositol 3,4-bisphosphate[PI(3,4)P₂] and phosphatidylinositol 3-monophosphate[PI(3)P] constitute second messengers in a variety of signal transduction pathways, including those essential to cell proliferation, adhesion, survival, cytoskeletal rearrangement and vesicle trafficking. PI(3)P is constitutively present in all cells and its levels do not change dramatically following agonist stimulation. Conversely, PI(3,4)P₂ and PI(3,4,5)P₃ are nominally absent in most cells but they rapidly accumulate on agonist stimulation.

The downstream effects of PI3K-produced 3-phosphoinositide second messengers are mediated by target molecules containing 3-phosphoinositide binding domains such as the pleckstrin homology (PH) domain and the recently identified FYVE and phox domains. Well-characterised protein targets for PI3K include PDK1 and protein kinase B (PKB). In addition, tyrosine kinases like Btk and Itk are dependent on PI3K activity.

The PI3K family of lipid kinases can be classified into three groups according to their physiological substrate specificity (Vanhaesebroeck et al., Trends in Biol. Sci., 1997, 22, 267). Class III PI3K enzymes phosphorylate PI alone. In contrast, Class II PI3K enzymes phosphorylate both PI and PI 4-phosphate[PI(4)P]. Class I PI3K enzymes phosphorylate PI, PI(4)P and PI 4,5-bisphosphate [PI(4,5)P₂], although only PI(4,5)P₂ is believed to be the physiological cellular substrate. Phosphorylation of PI(4,5)P₂ produces the lipid second messenger PI(3,4,5)P₃. More distantly related members of the lipid kinase superfamily are Class IV kinases such as mTOR (discussed above) and DNA-dependent kinase that phosphorylate serine/threonine residues within protein substrates. The most studied and understood of the PI3K lipid kinases are the Class I PI3K enzymes.

Class I PI3Ks are heterodimers consisting of a p110 catalytic subunit and a regulatory subunit. The family is further divided into Class Ia and Class Ib enzymes on the basis of regulatory partners and the mechanism of regulation. Class Ia enzymes consist of three distinct catalytic subunits (p110α, p110β and p110δ) that dimerise with five distinct regulatory subunits (p85α, p55α, p50α, p85β and p55γ), with all catalytic subunits being able to interact with all regulatory subunits to form a variety of heterodimers. Class Ia PI3Ks are generally activated in response to growth factor-stimulation of receptor tyrosine kinases via interaction of their regulatory subunit SH2 domains with specific phospho-tyrosine residues of activated receptor or adaptor proteins such as IRS-1. Both p110α and p110β are constitutively expressed in all cell types, whereas p 110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor systems (GPCRs) and its expression appears to be limited to leukocytes and cardiomyocytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 2, 489-501). For example, the p110α subunit is amplified in some tumours such as those of the ovary (Shayesteh et al., Nature Genetics, 1999, 21, 99-102) and cervix (Ma et al., Oncogene, 2000, 19, 2739-2744). More recently, activating mutations within the catalytic site of the p110α catalytic subunit have been associated with various other tumours such as those of the colorectal region and of the breast and lung (Samuels et al., Science, 2004, 304, 554). Tumour-related mutations in the p85α regulatory subunit have also been identified in cancers such as those of the ovary and colon (Philp et al., Cancer Research, 2001, 61, 7426-7429). In addition to direct effects, it is believed that activation of Class Ia PI3Ks contributes to tumourigenic events that occur upstream in signalling pathways, for example by way of ligand-dependent or ligand-independent activation of receptor tyrosine kinases, GPCR systems or integrins (Vara et al., Cancer Treatment Reviews, 2004, 30, 193-204). Examples of such upstream signalling pathways include over-expression of the receptor tyrosine kinase erbB2 in a variety of tumours leading to activation of PI3K-mediated pathways (Harari et al., Oncogene, 2000, 19, 6102-6114) and over-expression of the ras oncogene (Kauffmann-Zeh et al., Nature, 1997, 385, 544-548). In addition, Class Ia PI3Ks may contribute indirectly to tumourigenesis caused by various downstream signalling events. For example, loss of the effect of the PTEN tumour-suppressor phosphatase that catalyses conversion of PI(3,4,5)P₃ back to PI(4,5)P₂ is associated with a very broad range of tumours via deregulation of PI3K-mediated production of PI(3,4,5)P₃ (Simpson and Parsons, Exp. Cell Res., 2001, 264, 29-41). Furthermore, augmentation of the effects of other PI3K-mediated signalling events is believed to contribute to a variety of cancers, for example by activation of Akt (Nicholson and Anderson, Cellular Signalling, 2002, 14, 381-395).

In addition to a role in mediating proliferative and survival signalling in tumour cells, there is evidence that Class Ia PI3K enzymes contribute to tumourigenesis in tumour-associated stromal cells. For example, PI3K signalling is known to play an important role in mediating angiogenic events in endothelial cells in response to pro-angiogenic factors such as VEGF (Abid et al., Arterioscler. Thromb. Vasc. Biol., 2004, 24, 294-300). As Class I PI3K enzymes are also involved in motility and migration (Sawyer, Expert Opinion Investig. Drugs, 2004, 13, 1-19), PI3K enzyme inhibitors should provide therapeutic benefit via inhibition of tumour cell invasion and metastasis. In addition, Class I PI3K enzymes play an important role in the regulation of immune cells contributing to pro-tumourigenic effects of inflammatory cells (Coussens and Werb, Nature, 2002, 420, 860-867).

These findings suggest that pharmacological inhibitors of Class I PI3K enzymes will be of therapeutic value for the treatment of various diseases including different forms of the disease of cancer comprising solid tumours such as carcinomas and sarcomas and the leukaemias and lymphoid malignancies. In particular, inhibitors of Class I PI3K enzymes should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

PT3Kγ, the Class Ib PI3K, is activated by GPCRs, as was finally demonstrated in mice lacking the enzyme. Thus, neutrophils and macrophages derived from PT3Kγ-deficient animals failed to produce PI(3,4,5)P₃ in response to stimulation with various chemotactic substances (such as IL-8, C5a, fMLP and MIP-1a), whereas signalling through protein tyrosine kinase-coupled receptors to Class Ia PI3Ks was intact (Hirsch et al., Science, 2000, 287(5455), 1049-1053; Li et al., Science, 2002, 287(5455), 1046-1049; Sasaki et al., Science 2002, 287(5455), 1040-1046). Furthermore, PI(3,4,5)P₃-mediated phosphorylation of PKB was not initiated by these GPCR ligands in PT3Kγ-null cells. Taken together, the results demonstrated that, at least in resting haematopoietic cells, PI3Kγ is the sole PI3K isoform that is activated by GPCRs in vivo. When murine bone marrow-derived neutrophils and peritoneal macrophages from wild-type and PI3Kγ^{-/-} mice were tested in vitro, a reduced, but not completely abrogated, performance in chemotaxis and adherence assays was observed. However, this translated into a drastic impairment of IL-8 driven neutrophil infiltration into tissues (Hirsch et al., Science, 2000, 287(5455), 1049-1053.). Recent data suggest that PI3Kγ is involved in the path finding process rather than in the generation of mechanical force for motility, as random migration was not impaired in cells that lacked PI3Kγ (Hannigan et al., Proc. Nat. Acad. of Sciences of U.S.A., 2002, 99(6), 3603-8). Data linking PI3Kγ to respiratory disease pathology came with the demonstration that PI3Kγ has a central role in regulating endotoxin-induced lung infiltration and activation of neutrophils leading to acute lung injury (Yum et al., J. Immunology, 2001, 167(11), 6601-8). The fact that although PI3Kγ is highly expressed in leucocytes, its loss seems not to interfere with haematopoiesis, and the fact that PI3Kγ-null mice are viable and fertile further implicates this PI3K isoform as a potential drug target. Work with knockout mice also established that PI3Kγ is an essential amplifier of mast cell activation (Laffargue et al., Immunity, 2002, 16(3), 441-451).

Thus, in addition to tumourigenesis, there is evidence that Class I PI3K enzymes play a role in other diseases (Wymann et al., Trends in Pharmacological Science, 2003, 24, 366-376). Both Class Ia PI3K enzymes and the single Class Ib enzyme have important roles in cells of the immune system (Koyasu, Nature Immunology, 2003, 4, 313-319) and thus they are therapeutic targets for inflammatory and allergic indications. Recent reports demonstrate that mice deficient in PI3Kγ and PI3Kδ are viable, but have attenuated inflammatory and allergic responses (Ali et al., Nature, 2004, 431(7011), 1007-11). Inhibition of PI3K is also useful to treat cardiovascular disease via anti-inflammatory effects or directly by affecting cardiac myocytes (Prasad et al., Trends in Cardiovascular Medicine, 2003, 13, 206-212). Thus, inhibitors of Class I PI3K enzymes are expected to be of value in the prevention and treatment of a wide variety of diseases in addition to cancer.

Several compounds that inhibit PI3Ks and phosphatidylinositol (PI) kinase-related kinase (PI3KKs) have been identified, including wortmannin and the quercetin derivative LY294002. These compounds are reasonably specific inhibitors of PI3Ks and PI3KKs over other kinases but they lack potency and display little selectivity within the PI3K families.

Accordingly, it would be desirable to provide further effective mTOR and/or PI3K inhibitors for use in the treatment of cancer, inflammatory or obstructive airways diseases, immune or cardiovascular diseases.

Morpholino pyrimidine derivatives and PI3K inhibitors are known in the art.

International Patent Application WO 2004/048365 discloses compounds that possess PI3K enzyme inhibitory activity and are useful in the treatment of cancer. These compounds are arylamino- and heteroarylamino-substituted pyrimidines which differ from the compounds of the present invention by virtue of their arylamino- and heteroarylamino substituents. WO 2004/048365 does not disclose compounds with the —XR¹ substituents of the present invention. Inhibitors of PI3K activity useful in the treatment of cancer are also disclosed in European Patent Application 1 277 738 which mentions 4-morpholino-substituted bicyclic heteroaryl compounds such as quinazoline and pyrido[3,2-d]pyrimidine derivatives and 4-morpholino-substituted tricyclic heteroaryl compounds but not monocyclic pyrimidine derivatives.

WO2007/080382, WO2008/023180 and WO2008/023159 disclose compounds that possess mTOR and/or PI3K enzyme inhibitory activity and are useful in the treatment of cancer. WO2007/080382, WO2008/023180 and WO2008/023159 do not disclose compounds comprising a cyclic moiety in the linker group X in the group —XR¹.

A number of compounds such as 4-morpholin-4-yl-6-(phenylsulfonylmethyl)-2-pyridin-4-yl-pyrimidine and 4-{6-[(phenylsulfonyl)methyl]-2-pyridin-2-ylpyrimidin-4-yl}morpholine have been registered in the Chemical Abstracts database but no utility has been indicated and there is no suggestion that these compounds have mTOR and/or PI3K inhibitory activity or useful therapeutic properties.

Surprisingly, we have found that certain morpholino pyrimidine derivatives possess useful therapeutic properties. Without wishing to be bound by theoretical constraints, it is believed that the therapeutic usefulness of the derivatives is derived from their inhibitory activity against mTOR kinase and/or one or more PI3K enzyme (such as the Class Ia enzyme and/or the Class Ib enzyme). Because signalling pathways mediated by the PI3K/mTOR families have a central role in a number of cell processes including proliferation and survival, and because deregulation of these pathways is a causative factor in a wide spectrum of human cancers and other diseases, it is expected that the derivatives will be therapeutically useful. In particular, it is expected that the derivatives will have anti-proliferative and/or apoptotic properties which means that they will be useful in the treatement of proliferative disease such as cancer. The compounds of the present invention may also be useful in inhibiting the uncontrolled cellular proliferation which arises from various non-malignant diseases such as inflammatory diseases, obstructive airways diseases, immune diseases or cardiovascular diseases.

Generally, the compounds of the present invention possess potent inhibitory activity against mTOR kinase but the compound may also possess potent inhibitory activity against one or more PI3K enzyme (such as the Class Ia enzyme and/or the Class Ib enzyme).

In accordance with an aspect of the present invention, there is provided a compound of formula (I)

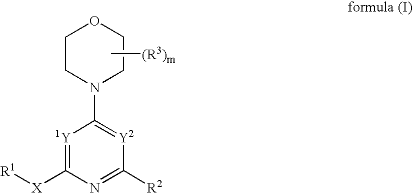

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, and —S(O)₂NR⁴CR⁶R⁷—;
R¹ is a group selected from hydrogen, C₁₋₆alkyl, C₂₋₆alkenyl, C₂-₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹⁹ is hydrogen, cyano or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl for use as a medicament in the treatment of proliferative disease.

In accordance with an aspect of the present invention, there is provided a compound of formula (I)

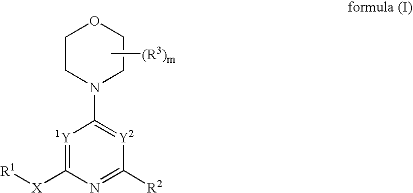

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, and —S(O)₂NR⁴CR⁶R⁷—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl for use as a medicament in the treatment of proliferative disease.

In accordance with another aspect of the present invention, there is provided a compound of formula (I)

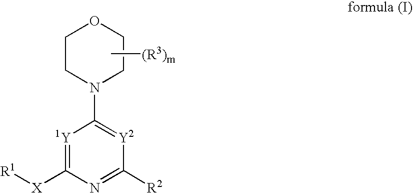

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
or X—R¹ is —CR⁶R⁷OH;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl for use as a medicament in the treatment of proliferative disease.

In accordance with another aspect of the present invention, there is provided a compound of formula (I)

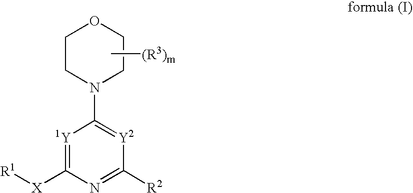

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷— and —S(O)₂NR⁴CR⁶R⁷—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
or X—R¹ is —CR⁶R⁷OH;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl for use as a medicament in the treatment of proliferative disease.

In accordance with another aspect of the present invention, there is provided the use of a compound of formula (I)

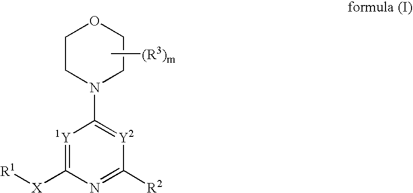

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹⁹ is hydrogen, cyano or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl in the manufacture of a medicament for use in the treatment of proliferative disease.

In accordance with another aspect of the present invention, there is provided the use of a compound of formula (I)

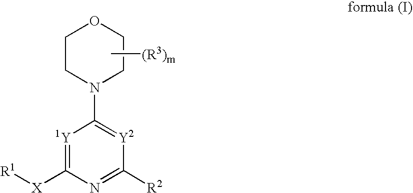

is or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl in the manufacture of a medicament for use in the treatment of proliferative disease.

In accordance with another aspect of the present invention, there is provided the use of a compound of formula (I)

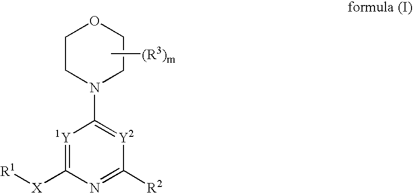

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
or X—R¹ is —CR⁶R⁷OH;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl in the manufacture of a medicament for use in the treatment of proliferative disease.

In accordance with another aspect of the present invention, there is provided the use of a compound of formula (I)

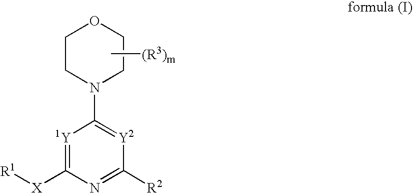

or a pharmaceutically acceptable salt; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷— and —S(O)₂NR⁴CR⁶R⁷—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
or X—R¹ is —CR⁶R⁷OH
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —R¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl in the manufacture of a medicament for use in the treatment of proliferative disease.

In accordance with a further aspect of the present invention, there is also provided a compound of formula (I)

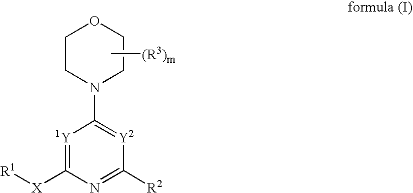

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —R¹³, —SOR¹³, —SO₂R¹³, —COR¹³, —CO₂R¹³, —CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³ , R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹⁹ is hydrogen, cyano or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In accordance with a further aspect of the present invention, there is also provided a compound of formula (I)

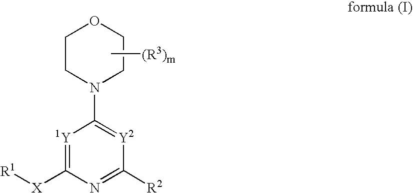

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR═CR⁵CR⁶R⁷—, —CR⁶R⁷CR⁵═CR⁴—, —C≡CCR⁶R⁷—, —CR⁶R⁷C≡C—, —NR⁴CR⁶R⁷, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —R¹³, ^{—}SOR¹³, —SO₂R¹³, —COR¹³, —CO₂R¹³, —CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In accordance with a further aspect of the present invention, there is also provided a compound of formula (I)

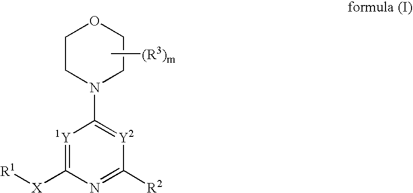

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹¹, —NR⁹COCONR¹⁰R¹⁵ and NR⁹SO₂R¹⁰;
or X—R¹ is —CR⁶R⁷OH;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —R¹³, ^{—}SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In accordance with a further aspect of the present invention, there is also provided a compound of formula (I)

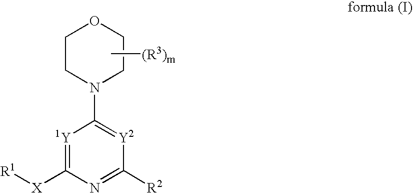

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵CR⁶R⁷—, —CR⁶R⁷CR⁵═CR⁴—, —C≡CCR⁶R⁷—, —CR⁶R⁷C≡C—, —NR⁴CR⁶R⁷, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷— and —S(O)₂NR⁴CR⁶R⁷—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;
or X—R¹ is —CR⁶R⁷OH;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —R¹³, ^{—}SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁹ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention. Solvates and mixtures thereof also form an aspect of the present invention. For example, a suitable solvate of a compound of formula (I) is, for example, a hydrate such as a hemi-hydrate, a mono-hydrate, a di-hydrate or a tri-hydrate or an alternative quantity thereof.

The present invention relates to the compounds of formula (I) as herein defined as well as to salts thereof Salts for use in pharmaceutical compositions will be pharmaceutically acceptable salts, but other salts may be useful in the production of the compounds of formula (I) and their pharmaceutically acceptable salts. Pharmaceutically acceptable salts of the invention may, for example, include acid addition salts of compounds of formula (I) as herein defined which are sufficiently basic to form such salts. Such acid addition salts include but are not limited to furmarate, methanesulfonate, hydrochloride, hydrobromide, citrate and maleate salts and salts formed with phosphoric and sulfuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl, 1,3-dioxolen-2-onylmethyl esters for example 5-methyl-1,3-dioxolen-2-onylmethyl, and C₁₋₆alkoxycarbonyloxyethyl esters for example 1-methoxycarbonyloxyethyl; and may be formed at any carboxy group in the compounds of this invention.

Suitable pharmaceutically acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl, benzoyl, phenylacetyl, substituted benzoyl and phenylacetyl; C₁₋₁₀alkoxycarbonyl (to give alkyl carbonate esters), for example ethoxycarbonyl; di-C₁₋₄alkylcarbamoyl and N-(di-C₁₋₄alkylaminoethyl)-N—C₁₋₄alkylcarbamoyl (to give carbamates); di-C₁₋₄alkylaminoacetyl and carboxyacetyl. Examples of ring substituents on phenylacetyl and benzoyl include aminomethyl, C₁₋₄alkylaminomethyl and di-(C₁₋₄alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆alkyl-CO—, wherein R^{A} is for example, benzyloxy-C₁₋₄alkyl, or phenyl. Suitable substituents on a phenyl group in such esters include, for example, 4-C₁₋₄piperazino-C₁₋₄alkyl, piperazino-C₁₋₄alkyl and morpholino-C₁₋₄alkyl.

The compounds of the formula (I) may be also be administered in the form of a prodrug which is broken down in the human or animal body to give a compound of the formula (I). Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

In this specification the generic term “C_{p-q}alkyl” includes both straight-chain and branched-chain alkyl groups. However references to individual alkyl groups such as “propyl” are specific for the straight chain version only (i.e. n-propyl and isopropyl) and references to individual branched-chain alkyl groups such as “tert-butyl” are specific for the branched chain version only.

The prefix C_{p-q} in C_{p-q}alkyl and other terms (where p and q are integers) indicates the range of carbon atoms that are present in the group, for example C₁₋₄alkyl includes C₁alkyl(methyl), C₂alkyl(ethyl), C₃alkyl(propyl as n-propyl and isopropyl) and C₄alkyl(n-butyl, sec-butyl, isobutyl and tert-butyl).

The term C_{p-q}alkoxy comprises —O—C_{p-q}alkyl groups.

The term C_{p-q}alkanoyl comprises —C(O)alkyl groups.

The term halo includes fluoro, chloro, bromo and iodo.

—Carbocyclyl” is a saturated, unsaturated or partially saturated monocyclic, bicyclic or tricyclic ring system containing from 3 to 14 ring atoms, wherein a ring CH₂ group may be replaced with a C═O group. “Carbocyclyl” includes “aryl”, “C_{p-q}cycloalkyl” and “C_{p-q}cycloalkenyl”.

“aryl” is an aromatic monocyclic, bicyclic or tricyclic carbcyclyl ring system.

“C_{p-q}cycloalkenyl” is an unsaturated or partially saturated monocyclic, bicyclic or tricyclic carbocyclyl ring system containing at least 1 C═C bond and wherein a ring CH₂ group may be replaced with a C═O group.

“C_{p-q}cycloalkyl” is a saturated monocyclic, bicyclic or tricyclic carbocyclyl ring system is and wherein a ring CH₂ group may be replaced with a C═O group.

“Heterocyclyl” is a saturated, unsaturated or partially saturated monocyclic, bicyclic or tricyclic ring system containing from 3 to 14 ring atoms of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group. “Heterocyclyl” includes “heteroaryl”, “cycloheteroalkyl” and “cycloheteroalkenyl”.

“Heteroaryl” is an aromatic monocyclic, bicyclic or tricyclic heterocyclyl, particularly having 5 to 10 ring atoms, of which 1, 2, 3 or 4 ring atoms are chosen from nitrogen, sulfur or oxygen where a ring nitrogen or sulfur may be oxidised.

“Cycloheteroalkenyl” is an unsaturated or partially saturated monocyclic, bicyclic or tricyclic heterocyclyl ring system, particularly having 5 to 10 ring atoms, of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

“Cycloheteroalkyl” is a saturated monocyclic, bicyclic or tricyclic heterocyclic ring system, particularly having 5 to 10 ring atoms, of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

This specification may make use of composite terms to describe groups comprising more than one functionality. Unless otherwise described herein, such terms are to be interpreted as is understood in the art. For example carbocyclylC_{p-q}alkyl comprises C_{p-q}alkyl substituted by carbocyclyl, heterocyclylC_{p-q}alkyl comprises C_{p-q}alkyl substituted by heterocyclyl, and bis(C_{p-q}alkyl)amino comprises amino substituted by 2 C_{p-q}alkyl groups which may be the same or different.

HaloC_{p-q}alkyl is a C_{p-q}alkyl group that is substituted by 1 or more halo substituents and particuarly 1, 2 or 3 halo substituents. Similarly, other generic terms containing halo such as haloC_{p-q}alkoxy may contain 1 or more halo substituents and particluarly 1, 2 or 3 halo substituents.

HydroxyC_{p-q}alkyl is a C_{p-q}alkyl group that is substituted by 1 or more hydroxyl substituents and particularly by 1, 2 or 3 hydroxy substituents. Similarly other generic terms containing hydroxy such as hydroxyC_{p-q}alkoxy may contain 1 or more and particularly 1, 2 or 3 hydroxy substituents.

C_{p-q}alkoxyC_{p-q}alkyl is a C_{p-q}alkyl group that is substituted by 1 or more C_{p-q}alkoxy substituents and particularly 1, 2 or 3 C_{p-q}alkoxy substituents. Similarly other generic terms containing C_{p-q}alkoxy such as C_{p-q}alkoxyC_{p-q}alkoxy may contain 1 or more C_{p-q}alkoxy substituents and particularly 1, 2 or 3 C_{p-q}alkoxy substituents.

Where optional substituents are chosen from “1 or 2”, from “1, 2, or 3” or from “1, 2, 3 or 4” groups or substituents it is to be understood that this definition includes all substituents being chosen from one of the specified groups i.e. all substitutents being the same or the substituents being chosen from two or more of the specified groups i.e. the substitutents not being the same.

Compounds of the present invention have been named with the aid of computer software (ACD/Name version 8.0).

“Proliferative disease(s)” includes malignant disease(s) such as cancer as well as non-malignant disease(s) such as inflammatory diseases, obstracutive airways diseases, immune diseases or cardiovascular diseases.

Suitable values for any R group or any part or substitutent for such groups include: for C₁₋₄alkyl: methyl, ethyl, propyl, butyl, 2-methylpropyl and tert-butyl;
for C₁₋₆alkyl: C₁₋₄alkyl, pentyl, 2,2-dimethylpropyl, 3-methylbutyl and hexyl;
for C₃₋₆cycloalkyl: cyclopropyl, cyclobutyl, cyclopentyl and cyclohexyl;
for C₃₋₆cycloalkylC₁₋₄alkyl: cyclopropylmethyl, cyclopropylethyl, cyclobutylmethyl, cyclopentylmethyl and cyclohexylmethyl;
for aryl: phenyl and naphthyl;
for arylC₁₋₄alkyl: benzyl, phenethyl, naphthylmethyl and naphthylethyl;
for carbocylyl: aryl, cyclohexenyl and C₃₋₆cycloalkyl;
for halo: fluoro, chloro, bromo and iodo;
for C₁₋₄alkoxy: methoxy, ethoxy, propoxy and isopropoxy;
for C₁₋₆alkoxy: C₁₋₄alkoxy, pentyloxy, 1-ethylpropoxy and hexyloxy;
for C₁₋₆alkanoyl: acetyl, propanoyl and 2-methylpropanoyl;
for heteroaryl: pyridinyl, imidazolyl, quinolinyl, cinnolyl, pyrimidinyl, thienyl, pyrrolyl, pyrazolyl, thiazolyl, thiazolyl, triazolyl, oxazolyl, isoxazolyl, furanyl, pyridazinyl, pyrazinyl, indolyl, benzofuranyl, dibenzofuranyl and benzothienyl;
for heteroarylC₁₋₄alkyl: pyrrolylmethyl, pyrrolylethyl, imidazolylmethyl, imidazolylethyl, pyrazolylmethyl, pyrazolylethyl, furanylmethyl, furanylethyl, thienylmethyl, theinylethyl, pyridinylmethyl, pyridinylethyl, pyrazinylmethyl, pyrazinylethyl, pyrimidinylmethyl, pyrimidinylethyl, pyrimidinylpropyl, pyrimidinylbutyl, imidazolylpropyl, imidazolylbutyl, quinolinylpropyl, 1,3,4-triazolylpropyl and oxazolylmethyl;
for heterocyclyl: heteroaryl, pyrrolidinyl, isoquinolinyl, quinoxalinyl, benzothiazolyl, benzoxazolyl, piperidinyl, piperazinyl, azetidinyl, morpholinyl, tetrahydroisoquinolinyl, tetrahydroquinolinyl, indolinyl, dihydro-2H-pyranyl and tetrahydrofuranyl.

It should be noted that examples given for terms used in the description are not limiting.

Particular values of m, X, ¹Y and Y², X, R¹, X—R¹, R², R³, R⁴, R⁵, R⁶, R⁷, R⁸, R⁹, R¹⁰, R¹¹, R¹², R¹³, R¹⁴, R^{17 , R}¹⁸ and R¹⁹ are as follows. Such values may be used idividually or in combination where appropriate, in connection with any aspect of the invention, or part thereof, and with any of the definitions, claims or embodiments defined herein.

m

In one aspect of the invention m is 0, 1, 2 or 3.

In another aspect m is 0, 1 or 2.

In a further aspect m is 0 or 1.

In yet another aspect m is 0 so that R³ is absent.

In yet another aspect m is 1 and R³ is methyl.

In yet another aspect m is 1 and R³ is hydroxymethyl.

In yet another aspect m is 1 and R³ is ethyl.

In yet another aspect m is 1 and R³ is dimethylcarbamoyl.

In yet another aspect m is 1 and R³ is carbamoyl.

In yet another aspect m is 2 and each R³ is methyl.

¹Y and Y²

In one aspect of the invention ¹Y is N and Y² is CR⁸.

In another aspect ¹Y is N and Y² is CH.

In yet another aspect ¹Y is CR⁸ and Y² is N.

In a further aspect ¹Y is CH or CF and Y² is N.

In yet a further aspect ¹Y is CH and Y² is N.

X

In one aspect of the invention X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷—.

In another aspect X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷.

In a further aspect X is a linker group selected from —NR⁴CR⁶R⁷—, —OCR⁶R⁷—, —SCR⁶R⁷—, —S(O)CR⁶R⁷— and —S(O)₂CR⁶R⁷—.

In a further aspect X is a linker group selected from —NR⁴CR⁶R⁷—, —OCR⁶R⁷—, —SCR⁶R⁷—, —S(O)CR⁶R⁷— and —S(O)₂CR⁶R⁷—.

In yet another aspect X is a linker group selected from —SCR⁶R⁷—, —S(O)CR⁶R⁷— and —S(O)₂CR⁶R⁷—.

In another aspect X is —SCR⁶R⁷— or —S(O)₂CR⁶R⁷—.

In another aspect X is —S(O)₂CR⁶R⁷—.

R¹

In one aspect of the invention R¹ is a group selected from C₁₋₄alkyl, C₃₋₁₀cycloalkyl, aryl, C₃₋₁₀cycloalkylC₁₋₄alkyl, arylC₁₋₄alkyl, cycloheteroalkyl, heteroaryl, cycloheteroalkylC₁₋₄alkyl, heteroarylC₁₋₄alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰.

In another aspect, R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thiadiazolyl, thiazolyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thiadiazolylmethyl, thiadiazolylethyl, thiazolylmethyl, thiazolylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰.

In another aspect, R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰.

In a further aspect, R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃.

In a further aspect, R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃.

In yet another aspect R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂C(O)NH₂, —CH₂C(O)NHMe, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-(trifluoromethyl)phenyl, 2-methylphenyl, 4-methylphenyl, 4-(2-hydroxyethylamino)phenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1-(difluoromethyl)-3,5-dimethylpyrazol-4-yl, 1,3-dimethylpyrazol-4-yl, pyridin-4-yl, pyridin-2-yl, 5-fluoropyridin-2-yl, 5-fluoropyridin-3-yl, thiazol-2-yl, 4-methylthiazol-2-yl, 4,5-dimethylthiazol-2-yl, 2,4-dimethylthiazol-5-yl, 5-methyl-1,3,4-thiadiazol-2-yl, terahydrofuran-3-yl and terahydropyran-4-yl.

In yet another aspect R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂OCH₃, —CH₂CH₂NHC(O)CH₃, —CH₂C(O)NH₂, —CH₂C(O)NHMe, phenyl, 4-fluorophenyl, 4-chlorophenyl, 2-chlorophenyl, 3,5-difluorophenyl, 2-(trifluoromethyl)phenyl, 2-methoxyphenyl, 2-methylphenyl, 4-(2-hydroxyethylamino)phenyl, 1H-imidazol-2-yl, 2-(dimethylcarbamoyl)pyridin-3-yl, 5-(dimethylcarbamoyl)pyridin-2-yl, 4-acetamidophenyl, 4-aminophenyl, pyridin-4-yl, pyridin-2-yl, 5-fluoropyridin-2-yl, 5-fluoropyridin-3-yl, 2-oxopyrolidin-3-yl, thiazol-2-yl, 4-methylthiazol-2-yl, 5-methyl-1,3,4-thiadiazol-2-yl and 3-methyl-1,3,4-thiadiazol-2-yl.

In yet another aspect R¹ is a group selected from methyl, isopropyl, cyclopropyl, cyclohexyl, —CH₂CH₂OH, —CH₂CH₂NHC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrolidin-3-yl, thiazol-2-yl, 4-methylthiazol-2-yl, and 3-methyl-1,3,4-thiadiazol-2-yl.

In yet another aspect R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂OCH₃, —CH₂CH₂NHC(O)CH₃, —CH₂C(O)NH₂, —CH₂C(O)NHMe, phenyl, 4-fluorophenyl, 4-chlorophenyl, 3,5-difluorophenyl, 2-(trifluoromethyl)phenyl, 4-(2-hydroxyethylamino)phenyl, 1H-imidazol-2-yl, 2-(dimethylcarbamoyl)pyridin-3-yl, 5-(dimethylcarbamoyl)pyridin-2-yl, pyridin-4-yl, pyridin-2-yl, 5-fluoropyridin-2-yl, 5-fluoropyridin-3-yl, thiazol-2-yl, 4-methylthiazol-2-yl and 5-methyl-1,3,4-thiadiazol-2-yl.

In yet another aspect R¹ is a group selected from methyl, ethyl, isopropyl, tert-butyl, cyclopropyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-methylphenyl, 4-methylphenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1,3-dimethylpyrazol-4-yl, pyridin-4-yl, pyridin-2-yl, 5-fluoropyridin-2-yl, thiazol-2-yl, 4-methylthiazol-2-yl, 4,5-dimethylthiazol-2-yl, 2,4-dimethylthiazol-5-yl and 5-methyl-1,3,4-thiadiazol-2-yl.

In yet another aspect R¹ is a group selected from methyl, ethyl, isopropyl, tert-butyl, cyclopropyl, —CH₂CH₂OH, —CH₂CH₂CH₂OH, —CH₂CH₂OCH₃, —CH₂CH₂NHC(O)CH₃, phenyl, 4-fluorophenyl, 4-chlorophenyl, 3,5-difluorophenyl, 1H-imidazol-2-yl, 2-(dimethylcarbamoyl)pyridin-3-yl, 5-(dimethylcarbamoyl)pyridin-2-yl, pyridin-4-yl, pyridin-2-yl, 5-fluoropyridin-2-yl, thiazol-2-yl, 4-methylthiazol-2-yl and 5-methyl-1,3,4-thiadiazol-2-yl.

In yet another aspect R¹ is a group selected from methyl, ethyl, cyclopropyl, —CH₂CH₂CH₂OH, phenyl, 2-fluorophenyl, 3-fluorophenyl, 4-fluorophenyl, 2-chlorophenyl, 2-methylphenyl, 5-fluoropyridin-2-yl, pyridin-2-yl, thiazol-2-yl and 4-methylthiazol-2-yl. In yet another aspect R¹ is a group selected from —CH₂CH₂CH₂OH, phenyl, 4-fluorophenyl, pyridin-2-yl, 5-fluoropyridin-2-yl, thiazol-2-yl and 4-methylthiazol-2-yl.

In yet another aspect R¹ is methyl.

In yet another aspect R¹ is ethyl.

In yet another aspect R¹ is cyclopropyl.

In yet another aspect R¹ is —CH₂CH₂CH₂OH.

In yet another aspect R¹ is phenyl.

In yet another aspect R¹ is 2-fluorophenyl.

In yet another aspect R¹ is 3-fluorophenyl.

In yet another aspect R¹ is 4-fluorophenyl.

In yet another aspect R¹ is 2-chlorophenyl.

In yet another aspect R¹ is 2-methylphenyl.

In yet another aspect R¹ is 5-fluoropyridin-2-yl

In yet another aspect R¹ is pyridin-2-yl.

In yet another aspect R¹ is thiazol-2-yl.

In yet another aspect R¹ is 4-methylthiazol-2-yl.

X—R¹

In one embodiment X—R¹ is —CR⁶R⁷OH.

R²

In one aspect of the invention R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R , —CONR¹¹R¹², —NR¹¹R¹², —NR¹¹COR¹², —NR¹¹COCONR¹²R¹⁶, —NR¹¹SO₂R¹¹, —NR¹⁷CONR¹⁸R¹⁹ and —NR¹⁷CSNR¹⁸R¹⁹.

In one aspect of the invention R² is selected from carbocyclyl or heterocyclyl which group is substituted by —NR¹⁷CONR¹⁸R¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from carbocyclyl or heterocyclyl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from carbocyclyl or heterocyclyl which group is substituted by —NHCONHR¹⁹ or —NHCSNHR¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is a group selected from 5 or 6 membered carbocyclyl or heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR SO₂R¹², —NR¹⁷CONR¹⁸R¹⁹ and —NR¹⁷CSNR¹⁸R¹⁹.

In one aspect of the invention R² is selected from 5 or 6 membered carbocyclyl or heterocyclyl which group is substituted by —NR¹⁷CONR¹⁸R¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from 5 or 6 membered carbocyclyl or heterocyclyl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from 5 or 6 membered carbocyclyl or heterocyclyl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from a 6 membered aryl and 5 or 6 membered heteroaryl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹.

In one aspect of the invention R² is selected from a 6 membered aryl and 5 or 6 membered heteroaryl which group is substituted by —NR¹⁷CONR¹⁸R¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from a 6 membered aryl and 5 or 6 membered heteroaryl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from a 6 membered aryl and 5 or 6 membered heteroaryl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from phenyl, pyrrolyl, imidazolyl, pyrazolyl, furanyl, thienyl, pyridinyl, pyrimidinyl, pyridazinyl, thiazolyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹.

In another aspect R² is selected from phenyl, pyrrolyl, imidazolyl, pyrazolyl, furanyl, thienyl, pyridinyl, pyrimidinyl, pyridazinyl, thiazolyl, which group is substituted by —NR¹⁷CONR¹⁸R¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In another aspect R² is selected from phenyl, pyrrolyl, imidazolyl, pyrazolyl, furanyl, thienyl, pyridinyl, pyrimidinyl, pyridazinyl, thiazolyl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In another aspect R² is selected from phenyl, pyrrolyl, imidazolyl, pyrazolyl, furanyl, thienyl, pyridinyl, pyrimidinyl, pyridazinyl, thiazolyl which group is substituted by —NHCONHR¹⁹ or —NHCSNHR¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from phenyl, pyrrolyl, imidazolyl, pyrazolyl, furanyl, thienyl, pyridinyl, pyrimidinyl, pyridazinyl, thiazolyl which group is optionally substituted by one or more substituent group independently selected from fluoro, methyl, methoxy, hydroxymethyl, cyanomethyl, —CONH₂, —CONHCH₃ and —CON(CH₃)₂, —NR¹¹COR¹², —NR¹¹SO₂R¹², —NR¹⁷CONR¹⁸R¹⁹ and —NR¹⁷CSNR¹⁸R¹⁹.

In another aspect R² is selected from phenyl, pyrrolyl, imidazolyl, pyrazolyl, furanyl, thienyl, pyridinyl, pyrimidinyl, pyridazinyl, thiazolyl which group is substituted by —NR¹⁷CONR¹⁸R¹⁹ or —NR¹⁷CSNR¹⁸R¹⁹ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is selected from phenyl, pyrrolyl, imidazolyl, pyrazolyl, furanyl, thienyl, pyridinyl, pyrimidinyl, pyridazinyl, thiazolyl which group is substituted by —NHCONR¹⁸R¹⁹ or —NHCSNR¹⁸R¹⁹ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is selected from phenyl, pyrrolyl, imidazolyl, pyrazolyl, furanyl, is thienyl, pyridinyl, pyrimidinyl, pyridazinyl, thiazolyl which group is substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂.

In one aspect of the invention R² is selected from phenyl, pyridinyl or pyrimidinyl which group is optionally substituted by one or more substituent group independently selected from fluoro, methyl, methoxy, hydroxymethyl, cyanomethyl, —CONH₂, —CONHCH₃ and —CON(CH₃)₂, —NR¹¹COR¹², —NR¹¹SO₂R¹², —NR¹⁷CONR¹⁸R¹⁹ and —NR¹⁷CSNR¹⁸R⁹.

In one aspect of the invention R² is selected from phenyl or pyridinyl which group is optionally substituted by one or more substituent group independently selected from fluoro, methyl, methoxy, hydroxymethyl, cyanomethyl, —CONH₂, —CONHCH₃ and —CON(CH₃)₂, —NR¹¹COR¹², —NR¹¹SO₂R¹², —NR¹⁷CONR¹⁸R¹⁹ and —NR¹⁷CSNR¹⁸R¹⁹.

In another aspect R² is phenyl, pyridinyl or pyrimidinyl substituted by —NR¹⁷CONR¹⁸R¹⁹ or —NR¹⁷CSNR¹⁸R¹⁹ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridinyl substituted by —NR¹⁷CONR¹⁸R¹⁹ or —NR¹⁷CSNR¹⁸R¹⁹ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridinyl substituted by —NHCONR¹⁸R¹⁹ or —NHCSNR¹⁸R¹⁹ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridinyl optionally substituted by —NR¹⁷CONR¹⁸R¹⁹ or —NR¹⁷CSNR¹⁸R¹⁹.

In another aspect R² is phenyl or pyridinyl optionally substituted by —NHCONR¹⁸R¹⁹ or —NHCSNR¹⁸R¹⁹.

In another aspect R² is phenyl or pyridinyl optionally substituted by —NHCONR¹⁸R¹⁹ or —NHCSNR¹⁸R¹⁹.

In another aspect R² is

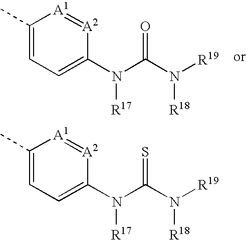

wherein A¹ and A² are selected from CH or N provided that at least one of A¹ or A² is CH.

In another aspect R² is

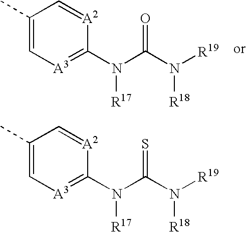

wherein A² and A³ are selected from CH or N.

In another aspect R² is

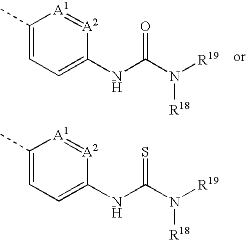

wherein A¹ and A² are selected from CH or N provided that at least one of A¹ or A² is CH.

In another aspect R² is

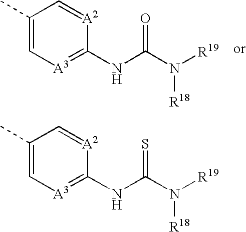

wherein A² and A³ are selected from CH or N.

In another aspect R² is

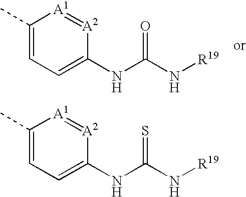

wherein A¹ and A² are selected from CH or N provided that at least one of A¹ or A² is CH.

In another aspect R² is

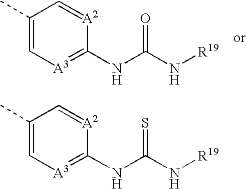

wherein A² and A³ are selected from CH or N.

In yet another aspect R² is 3-(hydroxymethyl)phenyl, 4-(hydroxymethyl)phenyl, 4-(cyanomethyl)phenyl, 3,4-dimethoxyphenyl, 3-fluoro-4-methoxyphenyl, 4-phenoxyphenyl, 3-pyrrolidin-1ylphenyl, 3-(aminocarbonyl)phenyl, 4-(dimethylaminocarbonyl)phenyl, furan-3-yl, thien-3-yl, 5-(hydroxymethyl)thien-2-yl, pyridin-2-yl, pyridin-4-yl, 2-methoxypyridin-5-yl, 2-methoxypyrimidin-5-yl, 2-methoxynaphth-6-yl, 5,7-diazabicyclo[4.3.0]nona-2,4,8,10-tetraenyl, azaindolyl, indol-5-yl, 1-methylindol-5-yl, quinolin-6-yl, benzimidazolyl, benzofuran-2-yl, dibenzofuran-1-yl and benzothien-3-yl.

In yet a further aspect R² is pyridin-2-yl, 3-hydroxyphenyl, 4-hydroxyphenyl, 3-hydroxymethylphenyl, 4-hydroxymethylphenyl or indol-5-yl.

In yet a further aspect R² is azaindolyl, indol-5-yl, benzimidazolyl, 3-hydroxyphenyl, 4-hydroxyphenyl, 3-hydroxymethylphenyl or 4-hydroxymethylphenyl

In another aspect R² is pyridin-2-yl.

In a further aspect R² is 3-hydroxyphenyl or 4-hydroxyphenyl.

In yet another aspect R² is 3-hydroxymethylphenyl or 4-hydroxymethylphenyl.

In yet a further aspect R² is indol-5-yl.

In one aspect R² is morpholinyl.

In another aspect R² is morpholino.

R³

Each R³ is independently selected from cyano, R¹³, and —CONR¹³R¹⁴, wherein R¹³ and R¹⁴ are independently hydrogen or a C₁₋₃alkyl which is optionally substituted by one or more substituent groups selected from halo, cyano, hydroxy and C₁₋₃alkoxy.

Each R³ is independently selected from hydrogen, C₁₋₃alkyl, hydroxyC₁₋₃alkyl, and —CONR¹³R¹⁴, wherein R¹³ and R¹⁴ are independently hydrogen or a C₁₋₃alkyl.

Each R³ is independently selected from hydrogen, methyl, ethyl, hydroxymethyl, carbamoyl and dimethylcarbamoyl.

R⁴

In one aspect of the invention R⁴ is hydrogen or methyl.

In another aspect R⁴ is hydrogen.

R⁴ and R¹

In another aspect of the invention, when X is —NR⁴CR⁶R⁷—, —NR⁴C(O)CR⁶R⁷—, —NR⁴C(O)NR⁵CR⁶R⁷— or —NR⁴S(O)₂CR⁶R⁷—, R¹ and R⁴ together with the atom or atoms to which they are attached form a 4- to 10-membered heterocyclic ring wherein 1, 2 or 3 ring carbon atoms is optionally replaced with N, O or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention, when X is —NR⁴CR⁶R⁷—, —NR⁴C(O)CR⁶R⁷—, —NR⁴C(O)NR⁵CR⁶R⁷— or —NR⁴S(O)₂CR⁶R⁷—, R¹ and R⁴ together with the atom or atoms to which they are attached form a 5-, 6- or 7-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention, when X is —NR⁴CR⁶R⁷—, —NR⁴C(O)CR⁶R⁷—, —NR⁴C(O)NR⁵CR⁶R⁷— or —NR⁴S(O)₂CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention, when X is —NR⁴CR⁶R⁷—, —NR⁴C(O)CR⁶R⁷—, —NR⁴C(O)NR⁵CR⁶R⁷— or —NR⁴S(O)₂CR⁶R⁷—, R¹ and R⁴ together with the atom or atoms to which they are attached form a morpholine or piperazine ring which ring is optionally substituted by one or more methyl groups.

In another aspect of the invention, when X is —NR⁴C(O)CR⁶R⁷—, R¹ and R⁴ together with the atom or atoms to which they are attached form a morpholine or piperazine ring which ring is optionally substituted by one or more methyl groups.

R⁵

In one aspect of the invention R⁵ is hydrogen or methyl.

In another aspect R⁵ is hydrogen.

In another aspect R⁵ is methyl.

R⁶ and R⁷

In one aspect of the invention R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R⁶ and R⁷ together with the carbon atom to which they are attached form a 3- to 6-membered carbocyclic ring or heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect R⁶ and R⁷ together with the carbon atom to which they are attached form a 3- to 6-membered carbocyclic ring.

In another aspect R⁶ and R⁷ together with the carbon atom to which they are attached form a 3- to 5-membered carbocyclic ring.

In another aspect R⁶ and R⁷ together with the carbon atom to which they are attached form a 3- to 4-membered carbocyclic ring.

In another aspect R⁶ and R⁷ together with the carbon atom to which they are attached form a 3-membered carbocyclic ring.

In another aspect R⁶ and R⁷ together with the carbon atom to which they are attached form a cyclopropyl, cyclobutyl, cyclopentyl, tetrahydropyranyl or piperidyl ring.

R⁸

In one aspect of the invention R⁸ is hydrogen or halo.

In another aspect R⁸ is hydrogen or fluoro.

In a further aspect R⁸ is hydrogen.

R⁹

In one aspect of the invention R⁹ is hydrogen or C₁₋₄alkyl optionally substituted by 1, 2 or 3 substituent groups selected from halo, cyano, nitro, hydroxy, C₁₋₄alkoxy, amino, C₁₋₄alkylamino and bis(C₁₋₄alkyl)amino.

In another aspect R⁹ is hydrogen or C₁₋₄alkyl optionally substituted by 1, 2 or 3 halo substituents.

In a further aspect R⁹ is hydrogen, methyl or trifluoromethyl.

R¹⁰

In one aspect of the invention R¹⁰ is hydrogen.

R¹¹

In one aspect of the invention R¹¹ is hydrogen or a group selected from C₁₋₄alkyl, aryl and cycloheteroalkyl which group is optionally substituted by 1, 2 or 3 groups selected from halo, hydroxy and cyano.

In another aspect R¹¹ is hydrogen, methyl optionally substituted with hydroxy or cyano, phenyl or pyrrolidinyl.

In another aspect R¹¹ is hydrogen or methyl.

R¹²

In one aspect of the invention R¹² is hydrogen or methyl.

R¹⁷

In one aspect of the invention R¹⁷ is hydrogen or a group selected from C₁₋₄alkyl, aryl and cycloheteroalkyl which group is optionally substituted by 1, 2 or 3 groups selected from halo, hydroxy and cyano.

In another aspect R¹⁷ is hydrogen, methyl optionally substituted with hydroxy or cyano, phenyl or pyrrolidinyl.

In another aspect R¹⁷ is hydrogen or methyl.

In another aspect R¹⁷ is hydrogen.

R¹⁸

In one aspect of the invention R¹⁸ is hydrogen or methyl.

In one aspect of the invention R¹⁸ is hydrogen

R¹⁹

In one aspect of the invention R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁹ is hydrogen or a group selected from C₁₋₆alkyl, C₃₋₆cycloakyl, phenyl, naphthyl, pyrrolyl, imidazolyl, isoxazolyl, pyrazolyl, furanyl, thienyl, pyridinyl, pyrimidinyl, pyridazinyl, azaindolyl, indolyl, quinolinyl, benzimidazolyl, benzofuranyl, dibenzofuranyl, benzothienyl, pyrrolidinyl, pyrazinyl, oxetanyl, dioxothiolanyl, thiazolyl, thiadiazolyl, phenylC₁₋₆alkyl, naphthylC₁₋₆alkyl, pyrrolylC₁₋₆alkyl, imidazolylC₁₋₆alkyl, isoxazolylC₁₋₆alkyl, pyrazolylC₁₋₆alkyl, furanylC₁₋₆alkyl, thienylC₁₋₆alkyl, pyridinylC₁₋₆alkyl, pyrimidinylC₁₋₆alkyl, pyridazinylC₁₋₆alkyl, azaindolylC₁₋₆alkyl, indolylC₁₋₆alkyl, quinolinylC₁₋₆alkyl, benzimidazolylC₁₋₆alkyl, benzofuranylC₁₋₆alkyl, dibenzofuranylC₁₋₆alkyl, benzothienylC₁₋₆alkyl, pyrrolidinylC₁₋₆alkyl, pyrazinylC₁₋₆alkyl, oxetanylC₁₋₆alkyl, dioxothiolanylC₁₋₆alkyl, thiazolylC₁₋₆alkyl and thiadiazolylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁹ is hydrogen or a group selected from C₁₋₆alkyl, C₃₋₆cycloakyl, phenyl, naphthyl, pyrrolyl, imidazolyl, isoxazolyl, pyrazolyl, furanyl, thienyl, pyridinyl, pyrimidinyl, pyridazinyl, azaindolyl, indolyl, quinolinyl, benzimidazolyl, benzofuranyl, dibenzofuranyl, benzothienyl, phenylC₁₋₆alkyl, naphthylC₁₋₆alkyl, pyrrolylC₁₋₆alkyl, imidazolylC₁₋₆alkyl, isoxazolylC₁₋₆alkyl, pyrazolylC₁₋₆alkyl, furanylC₁₋₆alkyl, thienylC₁₋₆alkyl, pyridinylC₁₋₆alkyl, pyrimidinylC₁₋₆alkyl, pyridazinylC₁₋₆alkyl, azaindolylC₁₋₆alkyl, indolylC₁₋₆alkyl, quinolinylC₁₋₆alkyl, benzimidazolylC₁₋₆alkyl, benzofuranylC₁₋₆alkyl, dibenzofuranylC₁₋₆alkyl, benzothienylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, phenyl, thienyl, dioxothiolanyl, imidazoylmethyl, isoxazolyl, oxazolyl, oxetanyl, pyrazinyl, pyrazolyl, pyrazolylmethyl, pyridinyl, pyrimidinyl, pyrrolidinyl, thiadiazolyl, thiazolyl and triazolyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyrazolylmethyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂SO₂Me, —CH₂CH(OH)CF₃, —CH₂CH₂CN, —CH₂CN, —CH₂CONMe₂, —CH₂CO₂H, 1-(methyl)cyclopropyl, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-(trifluoromethyl)phenyl, 4-fluorophenyl, 4-methoxyphenyl, 3,4-difluorophenyl, thien-2-yl, —CH₂CH₂(pyrrolidin-1-yl), —CH₂(imidazol-2-yl), —CH₂(imidazol-3-yl), 1-methylimidazol-4-yl, oxazolyl-2-yl, isoxazolyl-3-yl, is 6-oxo-1H-pryrdin-2-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, pyrimidin-2-yl, thiazol-2-yl, 1,2,4-thiadiazol-5-yl, 1-methylpyrazol-3-yl and 1H-pyrazol-3-yl.

In one aspect of the invention R¹⁹ is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂SO₂Me, —CH₂CH(OH)CF₃, —CH₂CH₂CN, —CH₂CN, —CH₂CONMe₂, —CH₂CO₂H, 1-(methyl)cyclopropyl, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl and 1-methylpyrazol-3-yl.

In one aspect of the invention R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, 13 C(CH₃)₂CH₂OH, —CH₂C(CH₃)₂OH, —CH₂C(CH₃)₂CH₂OH, —CH₂CH₂OH, —CH₂CH₂CH₂OH, —CH₂CHF₂, —CH₂CH₂SO₂Me, —CH₂CH(OH)CF₃, —CH₂CH₂CN, —CH₂CN, —CH₂CONMe₂, 1-(methyl)cyclopropyl, 1-(hydroxymethyl)cyclopropyl, phenyl, 4-methylphenyl, 4-chlorophenyl, 4-(trifluoromethyl)phenyl, 4-fluorophenyl, 4-methoxyphenyl, 3,4-difluorophenyl, thien-2-yl, —CH₂CH₂(pyrrolidin-1-yl), —CH₂(imidazol-2-yl), —CH₂(imidazol-3-yl), oxazolyl-2-yl, isoxazolyl-3-yl, 6-oxo-1H-pryrdin-2-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, pyrimidin-2-yl, thiazol-2-yl, 1,2,4-thiadiazol-5-yl, 1-methylpyrazol-3-yl and 1H-pyrazol-3-yl.

In one aspect of the invention R¹⁹ is hydrogen or a group selected from methyl, ethyl, is propyl, i-propyl, cyclopropyl, cyclobutyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂CN, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 5-methylisoxazol-3-yl, 1-methylpyrazol-4-yl, 5-methylpyrazin-2-yl, 6-methoxypryridin-3-yl, thiazol-2-yl, 1,2,4-thiadiazol-5-yl and 1-methylpyrazol-3-yl.

In one aspect of the invention R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-fluorophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, —CH₂(1-methylpyrazol-4-yl), 1-methylpyrazol-4-yl, —CH₂(1-methylpyrazol-4-yl), 6-methoxypryridin-3-yl, 5-fluoropyridin-2-yl, pyrimidin-2-yl, and 1H-pyrazol-3-yl.

In one aspect of the invention R¹⁹ is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CHF₂, —CH₂CH₂SO₂Me, —CH₂CH(OH)CF₃, —CH₂CH₂CN, —CH₂CN, —CH₂CONMe₂, 1-(methyl)cyclopropyl, 1-(hydroxymethyl)cyclopropyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl, and 1-methylpyrazol-3-yl.

In one aspect of the invention R¹⁹ is hydrogen or a group selected from methyl, ethyl, propyl, i-propyl, cyclopropyl, cyclobutyl, —CH₂(cyclopropyl), —CH₂CH₂NMe₂, —C(CH₃)₂CH₂OH, —CH₂CH₂OH, —CH₂CH₂CH₂OH, —CH₂CH₂CN, 1-(hydroxymethyl)cyclopropyl, phenyl, 4-methylphenyl, 4-chlorophenyl, 4-methoxyphenyl, 3,4-difluorophenyl, —CH₂CH₂(pyrrolidin-1-yl), —CH₂(imidazol-2-yl), oxazolyl-2-yl, isoxazolyl-3-yl, oxetan-3-yl, 5-methylisoxazol-3-yl, 1-methylpyrazol-4-yl, 5-methylpyrazin-2-yl, 6-methoxypryridin-3-yl, thiazol-2-yl, 1,2,4-thiadiazol-5-yl, and 1-methylpyrazol-3-yl.

In one aspect of the invention R¹⁹ is a group selected from methyl, ethyl, cyclopropyl, cyclobutyl, —CH(CH₃)CH₂OH, —CH₂CH₂OH, —CH₂CH₂CH₂OH, —CH₂CHF₂, —CH₂CH₂F, —CH₂CH₂CN, (1R)-2-hydroxy-1-methylethyl, (1S)-2-hydroxy-1-methylethyl, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl.

In one aspect of the invention R¹⁹ is a group selected from methyl, ethyl, cyclopropyl, cyclobutyl, —CH₂CH₂OH, —CH₂CH₂CH₂OH, —CH₂CH₂CN, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl.

In one aspect of the invention R¹⁹ is hydrogen or a group selected from methyl, ethyl, cyclopropyl, 1-methylpyrazol-4-yl, and —CH₂(1-methylpyrazol-4-yl).

In one aspect of the invention R¹⁹ is methyl.

In one aspect of the invention R¹⁹ is ethyl.

In one aspect of the invention R¹⁹ is cyclopropyl.

In one aspect of the invention R¹⁹ is cyclobutyl.

In one aspect of the invention R¹⁹ is —CH(CH₃)CH₂OH.

In one aspect of the invention R¹⁹ is —CH₂CH₂OH.

In one aspect of the invention R¹⁹ is —CH₂CH₂CH₂OH.

In one aspect of the invention R¹⁹ is —CH₂CHF₂.

In one aspect of the invention R¹⁹ is —CH₂CH₂F.

In one aspect of the invention R¹⁹ is —CH₂CH₂CN.

In one aspect of the invention R¹⁹ is (1R)-2-hydroxy-1-methylethyl.

In one aspect of the invention R¹⁹ is (1S)-2-hydroxy-1-methylethyl.

In one aspect of the invention R¹⁹ is —CH₂(imidazol-2-yl).

In one aspect of the invention R¹⁹ is oxazolyl-2-yl.

In one aspect of the invention R¹⁹ is isoxazolyl-3-yl.

In one aspect of the invention R¹⁹ is 1-methylpyrazol-4-yl.

In one aspect of the invention R¹⁹ is 5-methylpyrazin-2-yl.

In one aspect of the invention R¹⁹ is thiazol-2-yl.

In one aspect of the invention R¹⁹ is 1,2,4-thiadiazol-5-yl.

R¹⁸ and R¹⁹

In one aspect of the invention, R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention, R¹⁸ and R¹⁹ together with the nitrogen atom to which they are attached form a morpholine ring.

In one aspect of the invention, R¹⁸ and R¹⁹ together with the nitrogen atom to which they are attached form a 3-hydroxypyrrolidin-1-yl group.

In one aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is selected from cyano, R¹³, and —CONR¹³R¹⁴;
R⁴ and R⁵ are independently hydrogen or C₁₋₆alkyl;
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹³ and R¹⁴ are independently hydrogen or a C₁₋₃alkyl which is optionally substituted by one or more substituent groups selected from halo, cyano, hydroxy and C₁₋₃alkoxy; and
R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;

or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is selected from C₁₋₃alkyl, hydroxyC₁₋₃alkyl, and —CONR¹³R¹⁴;
R⁴ and R⁵ are independently hydrogen or C₁₋₆alkyl; or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹³ and R¹⁴ are independently hydrogen or a C₁₋₃alkyl; and
R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;

or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl. In another aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is methyl or ethyl;
R⁴ and R⁵ are independently hydrogen or C₁₋₆alkyl;
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen, cyano or a group selected from C₁₋₆alkyl, C₃-₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;

or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is methyl;
R⁴ and R⁵ are independently hydrogen or C₁₋₆alkyl;
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC, ₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen, cyano or a group selected from C₁₋₆alkyl, C₃-₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;

or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR^{R}¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is methyl;
R⁴ and R⁵ are independently hydrogen or C₁₋₆alkyl;
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from aryl and heteroaryl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is methyl;
R⁴ and R⁵ are independently hydrogen or C₁₋₆alkyl;
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶ R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is selected from aryl and heteroaryl which group is substituted by —NR¹⁷CONR¹⁸R¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
each R³, when present, is methyl;
R⁴ and R⁵ are independently hydrogen or C₁₋₆alkyl;
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷— and —S(O)₂CR⁶R⁷—;
R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thiadiazolyl, thiazolyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thiadiazolylmethyl, thiadiazolylethyl, thiazolylmethyl, thiazolylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from 5 or 6 membered aryl and heteroaryl which group is substituted by —NHCONR¹⁸R¹⁹ or —NHCSNR¹⁸R¹⁹and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
each R³, when present, is methyl;
R⁴ is hydrogen or C₁₋₆alkyl;
or, when X is —NR⁴CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (I), or a pharmaceutically acceptable salt thereof;
m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷— and —S(O)₂CR⁶R⁷—;
R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from 5 or 6 membered aryl and heteroaryl which group is substituted by —NHCONR¹⁸R¹⁹ or —NHCSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
each R³, when present, is methyl;
R⁴ is hydrogen or C₁₋₆alkyl;
or, when X is —NR⁴CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃-₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another particular class of compound of formula (I), or a pharmaceutically acceptable salt thereof;
m is 0 or 1;
¹Y is CH and Y² is N;
X is a —S(O)₂CR⁶R⁷— linker group;
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃;
or —XR¹ is —C(CH₃)₂OH or —CH₂OH;
R² is selected from phenyl, pyrrolyl, imidazolyl, pyrazolyl, furanyl, thienyl, pyridinyl, pyrimidinyl, pyridazinyl, thiazolyl which group is substituted by —NHCONHR¹⁹ or —NHCSNHR¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³, when present, is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another particular class of compound of formula (I), or a pharmaceutically acceptable salt thereof;
m is 0 or 1;
¹Y is CH and Y² is N;
X is a —S(O)₂CR⁶R⁷— linker group;
R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;
or —XR¹ is —C(CH₃)₂OH or —CH₂OH;
R² is selected from phenyl, pyrrolyl, imidazolyl, pyrazolyl, furanyl, thienyl, pyridinyl, pyrimidinyl, pyridazinyl, thiazolyl which group is substituted by —NHCONHR¹⁹ or —NHCSNHR¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³, when present, is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;
m is 0, 1 or 2;
X is a —S(O)₂CR⁶R⁷— linker group selected;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R³, when present, is methyl or ethyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
R¹⁹ is hydrogen, cyano or a group selected from methyl, ethyl, propyl, i-propyl, butyl, i-butyl, is t-butyl, pentyl, cyclopropyl, cyclobutyl, cyclopentyl, cyclohexyl, phenyl, thienyl, dioxothiolanyl, imidazoylmethyl, isoxazolyl, oxazolyl, oxetanyl, pyrazinyl, pyrazolyl, pyrazolylmethyl, pyridinyl, pyrimidinyl, pyrrolidinyl, thiadiazolyl, thiazolyl and triazolyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;
m is 1;
X is a —S(O)₂CR⁶R⁷— linker group selected;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R³ is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, is sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, phenyl, thienyl, dioxothiolanyl, imidazoylmethyl, isoxazolyl, oxazolyl, oxetanyl, pyrazinyl, pyrazolyl, pyrazolylmethyl, pyridinyl, pyrimidinyl, pyrrolidinyl, thiadiazolyl, thiazolyl and triazolyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;
m is 1;
X is a —S(O)₂CR⁶R⁷— linker group selected;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R³ is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyrazolylmethyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;
m is 1;
X is a —S(O)₂CR⁶R⁷— linker group;
¹Y is CH and Y² is N.
R¹ is a group selected from methyl, isopropyl, cyclopropyl, cyclohexyl, —CH₂CH₂OH, —CH₂CH₂NHC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrolidin-3-yl, thiazol-2-yl, 4-methylthiazol-2-yl, and 3-methyl-1,3,4-thiadiazol-2-yl;
R² is

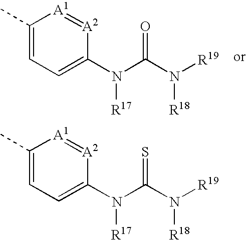

wherein A¹ and A² are selected from CH or N provided that at least one of A¹ or A² is CH;
   R¹⁷ is hydrogen;
   R¹⁸ is hydrogen;
   R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-flurophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, 1-methylpyrazol-4-yl, —CH₂(1-methylpyrazol-4-yl), —CH₂(1-methylpyrazol-4-yl), 6-methoxypryridin-3-yl, 5-fluoropyridin-2-yl, pyrimidin-2-yl, and 1H-pyrazol-3-yl;
R³ is methyl; and
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;
m is 0, 1 or 2;
X is a —S(O)₂CR⁶R⁷— linker group;
¹Y is CH and Y² is N.
R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂C(O)NH₂, —CH₂C(O)NHMe, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-(trifluoromethyl)phenyl, 2-methylphenyl, 4-methylphenyl, 4-(2-hydroxyethylamino)phenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1-(difluoromethyl)-3,5-dimethylpyrazol-4-yl, 1,3-dimethylpyrazol-4-yl, pyridin-4-yl, pyridin-2-yl, 5-fluoropyridin-2-yl, 5-fluoropyridin-3-yl, thiazol-2-yl, 4-methylthiazol-2-yl, 4,5-dimethylthiazol-2-yl, 2,4-dimethylthiazol-5-yl, 5-methyl-1,3,4-thiadiazol-2-yl, terahydrofuran-3-yl and terahydropyran-4-yl;
R² is

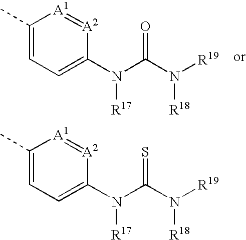

wherein A¹ and A² are selected from CH or N provided that at least one of A¹ or A² is CH;
   R¹⁷ is hydrogen;
   R¹⁸ is hydrogen;
   R¹⁹ is is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂SO₂Me, —CH₂CH(OH)CF₃, —CH₂CH₂CN, —CH₂CN, —CH₂CONMe₂, —CH₂CO₂H, 1-(methyl)cyclopropyl, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl and 1-methylpyrazol-3-yl;
R³, when present, is methyl or ethyl; and
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;
m is 1;
X is a —S(O)₂CR⁶R⁷— linker group;
¹Y is CH and Y² is N.
R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂OCH₃, —CH₂CH₂NHC(O)CH₃, —CH₂C(O)NH₂, —CH₂C(O)NHMe, phenyl, 4-fluorophenyl, 4-chlorophenyl, 3,5-difluorophenyl, 2-(trifluoromethyl)phenyl, 4-(2-hydroxyethylamino)phenyl, 1H-imidazol-2-yl, 2-(dimethylcarbamoyl)pyridin-3-yl, 5-(dimethylcarbamoyl)pyridin-2-yl, pyridin-4-yl, pyridin-2-yl, 5-fluoropyridin-2-yl, 5-fluoropyridin-3-yl, thiazol-2-yl, 4-methylthiazol-2-yl and 5-methyl-1,3,4-thiadiazol-2-yl;
R² is

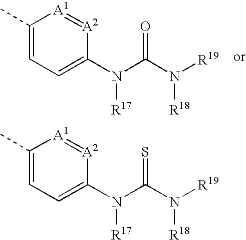

wherein A¹ and A² are selected from CH or N provided that at least one of A¹ or A² is CH;
   R¹⁷ is hydrogen;
   R¹⁸ is hydrogen;
   R¹⁹ is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CHF₂, —CH₂CH₂SO₂Me, —CH₂CH(OH)CF₃, —CH₂CH₂CN, —CH₂CN, —CH₂CONMe₂, 1-(methyl)cyclopropyl, 1-(hydroxymethyl)cyclopropyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl, and 1-methylpyrazol-3-yl;
R³ is methyl; and
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention there is provided a subset of compounds of formula (IA), (IB) or (IC)

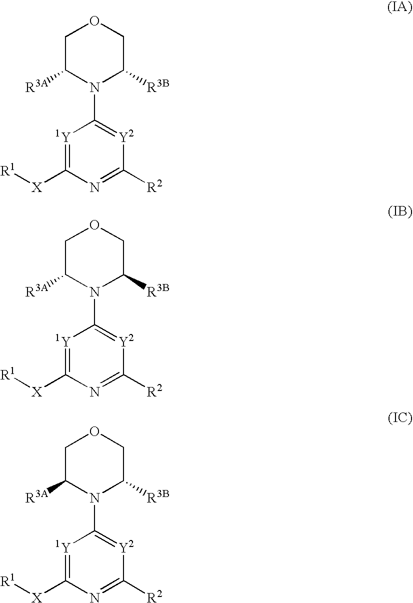

or a pharmaceutically acceptable salt thereof;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶ R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from aryl and heteroaryl which group is substituted by —NR¹⁷CONR¹⁸R¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R⁴ and R⁵ are independently hydrogen or C₁₋₆alkyl
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
when R^{3A} is hydrogen, R^{3B} is hydrogen, methyl, ethyl, hydroxymethyl, dimethylcarbamoyl or carbamoyl; or
when R^{3A} is methyl, R^{3B} is methyl.

In one aspect of the invention there is provided a subset of compounds of formula (IA), (IB) or (IC)

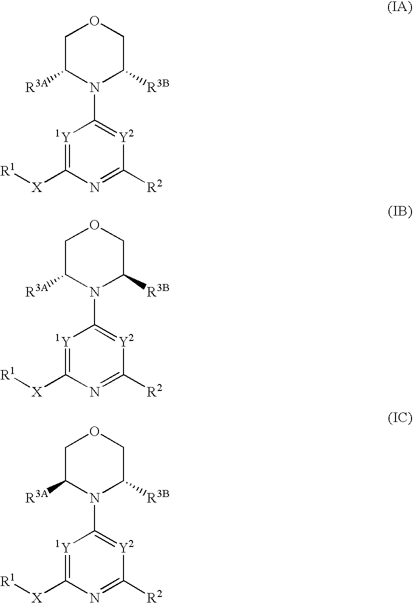

or a pharmaceutically acceptable salt thereof;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from aryl and heteroaryl which group is substituted by —NR¹⁷CONR¹⁸R¹⁹ or —NR¹⁷CSNR¹⁸R¹⁹and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R^{3A} and R^{3B} independently is hydrogen, methyl or ethyl;
R⁴ and R⁵ are independently hydrogen or C₁₋₆alkyl
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₁₆alkyl)aminoC₁₋₆alkyl, bis(C₁₋₆alkyl)aminoC₁₋₆alkyl, cyanoC₁₋₁₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloalkyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention there is provided a subset of compounds of formula (Ia) or (Ib)

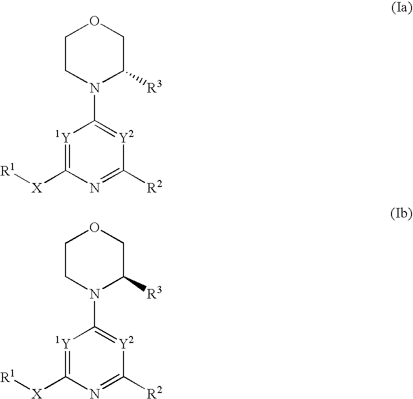

or a pharmaceutically acceptable salt thereof,
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from aryl and heteroaryl which group is substituted by —NR¹⁷CONR¹⁸R¹⁹ or —NR¹⁷CSNR¹⁸R¹⁹and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is hydrogen, methyl or ethyl;
R⁴ and R⁵ are independently hydrogen or C₁₋₆alkyl or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloalkyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;

or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention there is provided a subset of compounds of formula (Ia) or (Ib)

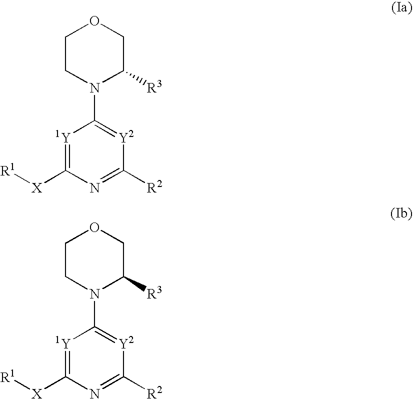

or a pharmaceutically acceptable salt thereof,
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from aryl and heteroaryl which group is substituted by —NR¹⁷CONR¹⁸R¹⁹ or —NR¹⁷CSNR¹⁸R¹⁹and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁴ and R⁵ are independently hydrogen or C₁₋₆alkyl
or, when X is —NR⁴CR⁶R⁷—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (Ia) or (Ib)

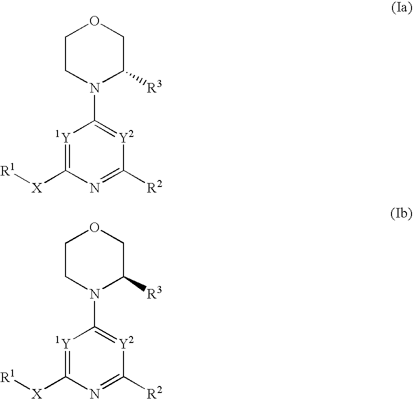

or a pharmaceutically acceptable salt thereof,
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷— and —S(O)₂CR⁶R⁷—;
R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thiadiazolyl, thiazolyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thiadiazolylmethyl, thiadiazolylethyl, thiazolylmethyl, thiazolylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from 5 or 6 membered aryl and heteroaryl which group is substituted by —NHCONR¹⁸R¹⁹ or —NHCSNR¹⁸R¹⁹and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁴ is hydrogen or C₁₋₆alkyl;
or, when X is —NR⁴CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₁₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₁₆alkylsulfonyl, C₁₋₁₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;

or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₋₁₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (Ia) or (Ib)

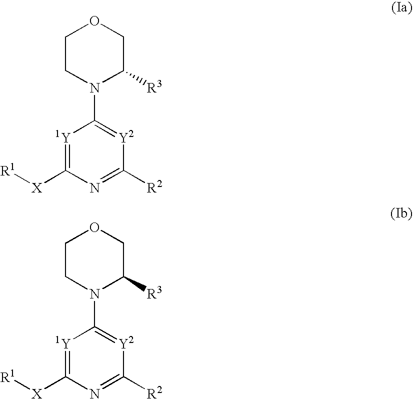

or a pharmaceutically acceptable salt thereof,
¹Y and Y² are independently N or CR⁸ provided that one of ¹Y and Y² is N and the other is CR⁸;
X is a linker group selected from —NR⁴CR⁶R⁷, —OCR⁶R⁷—, —SCR⁶R⁷—, —S(O)CR⁶R⁷— and —S(O)₂CR⁶R⁷—;
R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰;
or X—R¹ is —C(CH₃)₂OH or —CH₂OH;
R² is selected from 5 or 6 membered aryl and heteroaryl which group is substituted by —NHCONR¹⁸R¹⁹ or —NHCSNR¹⁸R¹⁹and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁴ is hydrogen or C₁₋₆alkyl;
or, when X is —NR⁴CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another particular class of compound of formula (Ia) or (Ib),

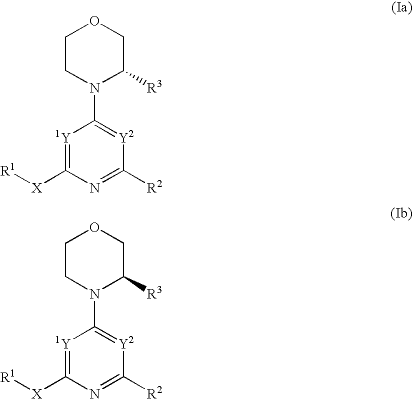

or a pharmaceutically acceptable salt thereof,
¹Y is CH and Y² is N;
X is a —S(O)₂CR⁶R⁷— linker group;
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃; or —XR¹ is —C(CH₃)₂OH or —CH₂OH;
R² is selected from phenyl, pyrrolyl, imidazolyl, pyrazolyl, furanyl, thienyl, pyridinyl, pyrimidinyl, pyridazinyl, thiazolyl which group is substituted by —NHCONHR¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and

R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another particular class of compound of formula (Ia) or (Ib),

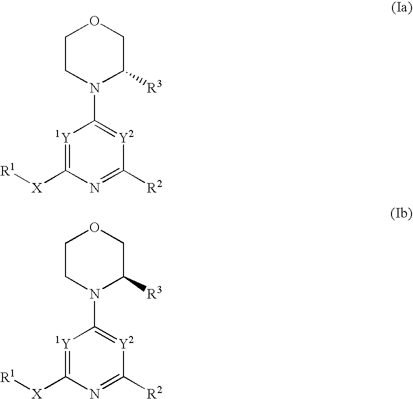

or a pharmaceutically acceptable salt thereof,
¹Y is CH and Y² is N;
X is a —S(O)₂CR⁶R⁷— linker group;
R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;
or —XR¹ is —C(CH₃)₂OH or —CH₂OH;
R² is selected from phenyl, pyrrolyl, imidazolyl, pyrazolyl, furanyl, thienyl, pyridinyl, pyrimidinyl, pyridazinyl, thiazolyl which group is substituted by —NHCONHR¹⁹ or —NR¹⁷CSNR¹⁸R¹⁹ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁹ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (IA), (IB) or (IC)

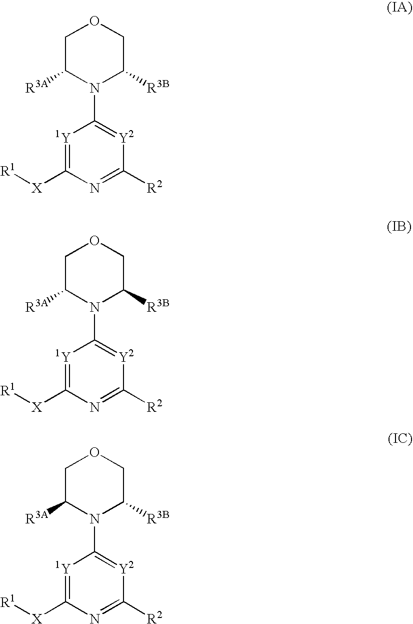

or a pharmaceutically acceptable salt thereof,
X is a —S(O)₂CR⁶R⁷— linker group;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R^{3A} and R^{3B} each independently is hydrogen, methyl or ethyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, phenyl, thienyl, dioxothiolanyl, imidazoylmethyl, isoxazolyl, oxazolyl, oxetanyl, pyrazinyl, pyrazolyl, pyrazolylmethyl, pyridinyl, pyrimidinyl, pyrrolidinyl, thiadiazolyl, thiazolyl and triazolyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
when R^{3A} is hydrogen, R^{3B} is hydrogen, methyl, ethyl, hydroxymethyl, dimethylcarbamoyl or carbamoyl; or
when R^{3A} is methyl, R^{3B} is methyl.

In a further particular class of compound of formula (Ia) or (Ib)

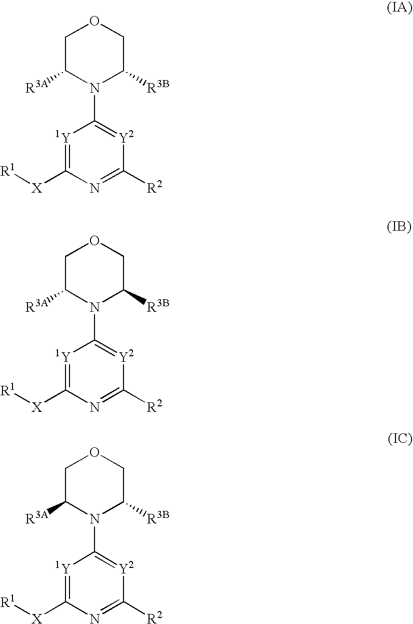

or a pharmaceutically acceptable salt thereof,
X is a —S(O)₂CR⁶R⁷— linker group;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R^{3A} and R^{3B} each independently is hydrogen, methyl or ethyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, phenyl, thienyl, dioxothiolanyl, imidazoylmethyl, isoxazolyl, oxazolyl, oxetanyl, pyrazinyl, pyrazolyl, pyrazolylmethyl, pyridinyl, pyrimidinyl, pyrrolidinyl, thiadiazolyl, thiazolyl and triazolyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

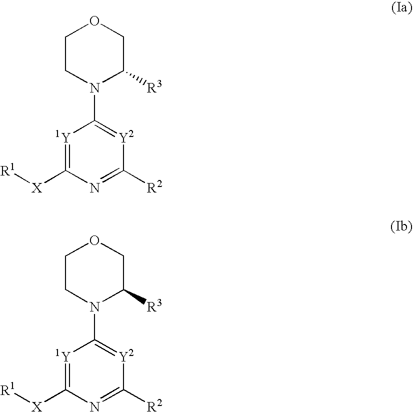

or a pharmaceutically acceptable salt thereof,
X is a —S(O)₂CR⁶R⁷— linker group;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R³ is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, phenyl, thienyl, dioxothiolanyl, imidazoylmethyl, isoxazolyl, oxazolyl, oxetanyl, pyrazinyl, pyrazolyl, pyrazolylmethyl, pyridinyl, pyrimidinyl, pyrrolidinyl, thiadiazolyl, thiazolyl and triazolyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

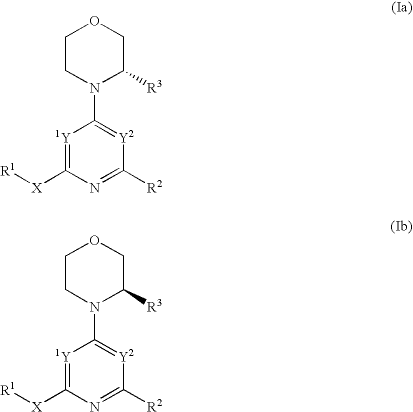

or a pharmaceutically acceptable salt thereof,
X is a —S(O)₂CR⁶R⁷— linker group;
¹Y is CH and Y² is N.
R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;
R² is phenyl or pyridinyl substituted by —NHCONHR¹⁹ or —NHCSNHR¹⁹ and optionally substituted by one or more substituent group independently selected from fluoro, methyl, methoxy, hydroxymethyl, cyanomethyl, —CONH₂, —CONHCH₃ and —CON(CH₃)₂;
R³ is methyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; and
R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyrazolylmethyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

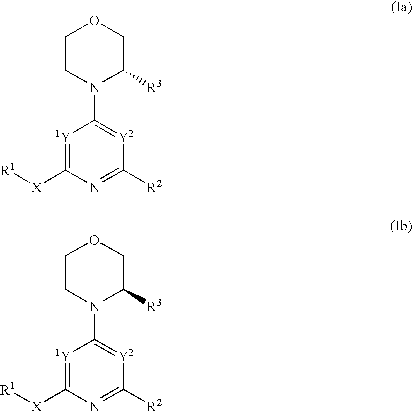

or a pharmaceutically acceptable salt thereof,
m is 1;
X is a —S(O)₂CR⁶R⁷— linker group;
¹Y is CH and Y² is N.
R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂OCH₃, —CH₂CH₂NHC(O)CH₃, —CH₂C(O)NH₂, —CH₂C(O)NHMe, phenyl, 4-fluorophenyl, 4-chlorophenyl, 2-chlorophenyl, 3,5-difluorophenyl, 2-(trifluoromethyl)phenyl, 4-(2-hydroxyethylamino)phenyl, 2-methoxyphenyl, 2-methylphenyl, 1H-imidazol-2-yl, 2-(dimethylcarbamoyl)pyridin-3-yl, 5-(dimethylcarbamoyl)pyridin-2-yl, 4-acetamidophenyl, 4-aminophenyl, pyridin-4-yl, pyridin-2-yl, 5-fluoropyridin-2-yl, 5-fluoropyridin-3-yl, 2-oxopyrolidin-3-yl, thiazol-2-yl, 4-methylthiazol-2-yl, 5-methyl-1,3,4-thiadiazol-2-yl and 3-methyl-1,3,4-thiadiazol-2-yl;
R² is

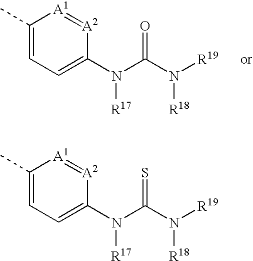

wherein A¹ and A² are selected from CH or N provided that at least one of A¹ or A² is CH;
   R¹⁷ is hydrogen;
   R¹⁸ is hydrogen; and
   R¹⁹ is hydrogen, cyano or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂C(CH₃)₂OH,
   —CH₂C(CH₃)₂CH₂OH, —CH₂CH₂OH, —CH₂CH₂CH₂OH, —CH₂CHF₂, —CH₂CH₂SO₂Me, —CH₂CH(OH)CF₃, —CH₂CH₂CN, —CH₂CN, —CH₂CONMe₂, 1-(methyl)cyclopropyl, 1-(hydroxymethyl)cyclopropyl, phenyl, 4-methylphenyl, 4-chlorophenyl, 4-(trifluoromethyl)phenyl, 4-fluorophenyl, 4-methoxyphenyl, 3,4-difluorophenyl, thien-2-yl, —CH₂CH₂(pyrrolidin-1-yl), —CH₂(imidazol-2-yl), —CH₂(imidazol-3-yl), oxazolyl-2-yl, isoxazolyl-3-yl, 6-oxo-1H-pryrdin-2-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, pyrimidin-2-yl, thiazol-2-yl, 1,2,4-thiadiazol-5-yl, 1-methylpyrazol-3-yl and 1H-pyrazol-3-yl;
R³ is methyl; and
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC-₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

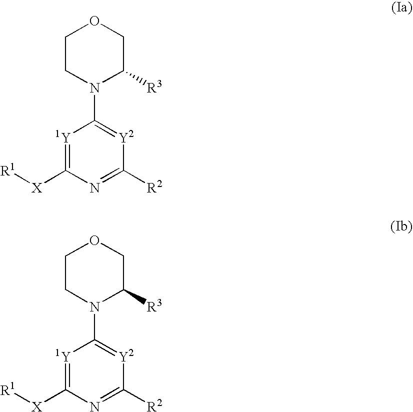

or a pharmaceutically acceptable salt thereof,
m is 1;
X is a —S(O)₂CR⁶R⁷— linker group;
¹Y is CH and Y² is N.
R¹ is a group selected from methyl, isopropyl, cyclopropyl, cyclohexyl, —CH₂CH₂OH, —CH₂CH₂NHC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrolidin-3-yl, thiazol-2-yl, 4-methylthiazol-2-yl, and 3-methyl-1,3,4-thiadiazol-2-yl;
R² is

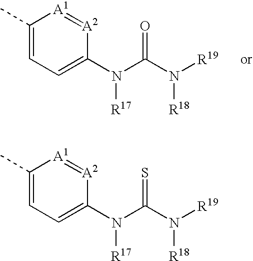

wherein A¹ and A² are selected from CH or N provided that at least one of A¹ or A² is CH;
   R¹⁷ is hydrogen;
   R¹⁸ is hydrogen; and
   R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-flurophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, 1-methylpyrazol-4-yl, —CH₂(1-methylpyrazol-4-yl), —CH₂(1-methylpyrazol-4-yl), 6-methoxypryridin-3-yl, 5-fluoropyridin-2-yl, pyrimidin-2-yl, and 1H-pyrazol-3-yl;
R³ is methyl; and
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC-₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

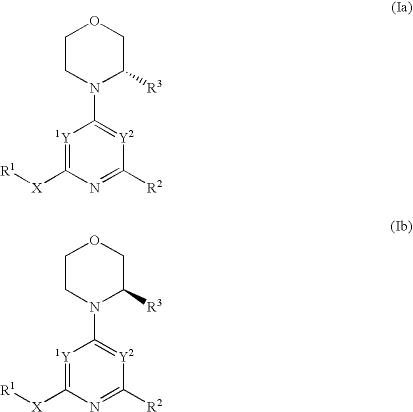

or a pharmaceutically acceptable salt thereof,
m is 1;
X is a —S(O)₂CR⁶R⁷— linker group;
¹Y is CH and Y² is N.
R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂OCH₃, —CH₂CH₂NHC(O)CH₃, —CH₂C(O)NH₂, —CH₂C(O)NHMe, phenyl, 4-fluorophenyl, 4-chlorophenyl, 3,5-difluorophenyl, 2-(trifluoromethyl)phenyl, 4-(2-hydroxyethylamino)phenyl, 1H-imidazol-2-yl, 2-(dimethylcarbamoyl)pyridin-3-yl, 5-(dimethylcarbamoyl)pyridin-2-yl, pyridin-4-yl, pyridin-2-yl, 5-fluoropyridin-2-yl, 5-fluoropyridin-3-yl, thiazol-2-yl, 4-methylthiazol-2-yl and 5-methyl-1,3,4-thiadiazol-2-yl;
R² is

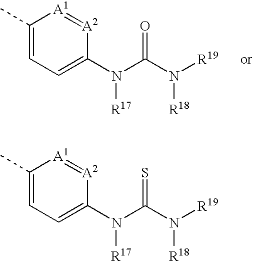

wherein A¹ and A² are selected from CH or N provided that at least one of A¹ or A² is CH;
   R¹⁷ is hydrogen;
   R¹⁸ is hydrogen; and
   R¹⁹ is is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CHF₂, —CH₂CH₂SO₂Me, —CH₂CH(OH)CF₃, —CH₂CH₂CN, —CH₂CN, —CH₂CONMe₂, 1-(methyl)cyclopropyl, 1-(hydroxymethyl)cyclopropyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl, and 1-methylpyrazol-3-yl;
R³ is methyl; and
R⁶ and R⁷ together with the carbon atom to which they are attached form a cyclopropyl, cyclobutyl, cyclopentyl, tetrahydropyranyl or piperidyl ring.

In a further particular class of compound of formula (IA), (IB) or (IC)

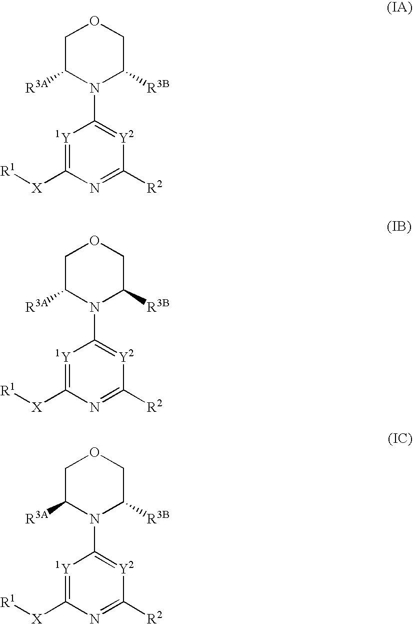

or a pharmaceutically acceptable salt thereof,
X is a —S(O)₂CR⁶R⁷— linker group;
¹Y is CH and Y² is N;
R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂C(O)NH₂, —CH₂C(O)NHMe, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 5 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-(trifluoromethyl)phenyl, 2-methylphenyl, 4-methylphenyl, 4-(2-hydroxyethylamino)phenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1-(difluoromethyl)-3,5-dimethylpyrazol-4-yl, 1,3-dimethylpyrazol-4-yl, pyridin-4-yl, pyridin-2-yl, 5-fluoropyridin-2-yl, 5-fluoropyridin-thiazol-2-yl, 4-methylthiazol-2-yl, 4,5-dimethylthiazol-2-yl, 2,4-dimethylthiazol-5-yl, 5-methyl-1,3,4-thiadiazol-2-yl, terahydrofuran-3-yl and terahydropyran-4-yl;
R² is

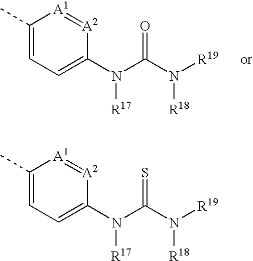

wherein A¹ and A² are selected from CH or N provided that at least one of A¹ or A² is CH;
   R¹⁷ is hydrogen;
   R¹⁸ is hydrogen; and
   R¹⁹ is is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂SO₂Me, —CH₂CH(OH)CF₃, —CH₂CH₂CN, —CH₂CN, —CH₂CONMe₂, —CH₂CO₂H, 1-(methyl)cyclopropyl, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl and 1-methylpyrazol-3-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl, ethyl, hydroxymethyl, dimethylcarbamoyl or carbamoyl; or
when R^{3A} is methyl, R^{3B} is methyl.

In a further particular class of compound of formula (IA), (IB) or (IC)

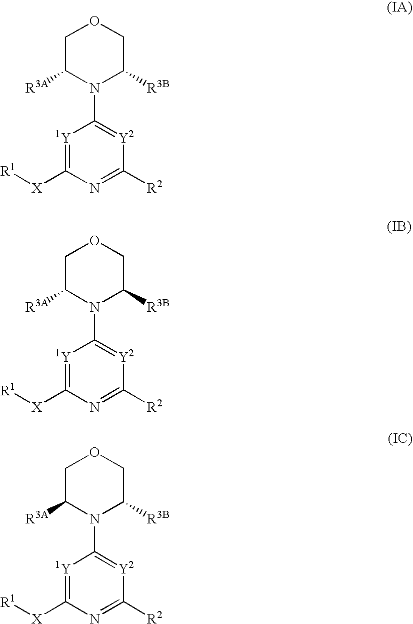

or a pharmaceutically acceptable salt thereof,
X is a —S(O)₂CR⁶R⁷— linker group;
¹Y is CH and Y² is N;
R¹ is a group selected from methyl, ethyl, isopropyl, tert-butyl, cyclopropyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-methylphenyl, 4-methylphenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1,3-dimethylpyrazol-4-yl, pyridin-4-yl, pyridin-2-yl, 5-fluoropyridin-2-yl, thiazol-2-yl, 4-methylthiazol-2-yl, 4,5-dimethylthiazol-2-yl, 2,4-dimethylthiazol-5-yl and 5-methyl-1,3,4-thiadiazol-2-yl;
R² is

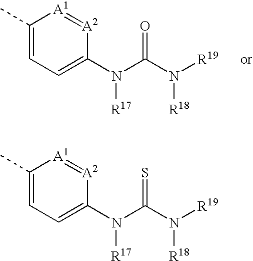

wherein A¹ and A² are selected from CH or N provided that at least one of A¹ or A² is CH;
   R¹⁷ is hydrogen;
   R¹⁸ is hydrogen; and
   R¹⁹ is hydrogen or a group selected from methyl, ethyl, propyl, i-propyl, cyclopropyl, cyclobutyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂CN, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 5-methylisoxazol-3-yl, 1-methylpyrazol-4-yl, 5-methylpyrazin-2-yl, 6-methoxypryridin-3-yl, thiazol-2-yl, 1,2,4-thiadiazol-5-yl and 1-methylpyrazol-3-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl or ethyl; or
when R^{3A} is methyl, R^{3B} is methyl.

In a further particular class of compound of formula (IA), (IB) or (IC)

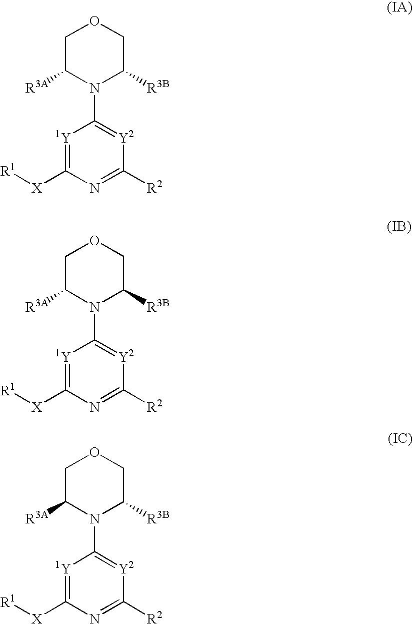

or a pharmaceutically acceptable salt thereof,
X is a —S(O)₂CR⁶R⁷— linker group;
¹Y is CH and Y² is N;
R¹ is a group selected from methyl, ethyl, cyclopropyl, —CH₂CH₂CH₂OH, phenyl, 2-fluorophenyl, 3-fluorophenyl, 4-fluorophenyl, 2-chlorophenyl, 2-methylphenyl, 5-fluoropyridin-2-yl, pyridin-2-yl, thiazol-2-yl and 4-methylthiazol-2-yl;
R² is

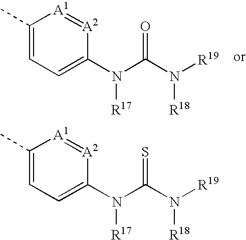

wherein A¹ and A² are selected from CH or N provided that at least one of A¹ or A² is CH;
   R¹⁷ is hydrogen;
   R¹⁸ is hydrogen; and
   R¹⁹ is a group selected from methyl, ethyl, cyclopropyl, cyclobutyl, —CH(CH₃)CH₂OH, —CH₂CH₂OH, —CH₂CH₂CH₂OH, —CH₂CHF₂, —CH₂CH₂F, —CH₂CH₂CN, (1R)-2-hydroxy-1-methylethyl, (1S)-2-hydroxy-1-methylethyl, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl or ethyl; or
when R^{3A} is methyl, R^{3B} is methyl.

In a further particular class of compound of formula (IA), (IB) or (IC)

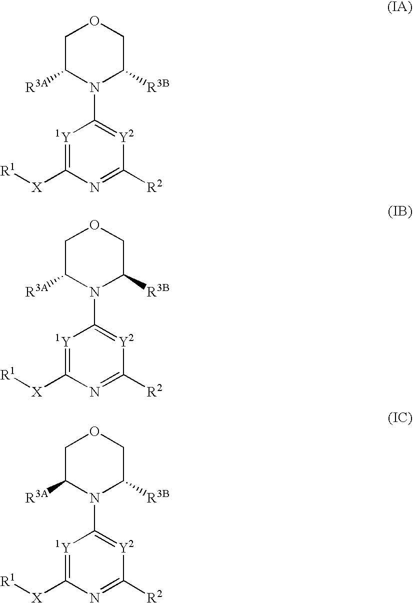

or a pharmaceutically acceptable salt thereof,
X is a —S(O)₂CR⁶R⁷— linker group;
¹Y is CH and Y² is N;
R¹ is a group selected from methyl, ethyl, isopropyl, tert-butyl, cyclopropyl, cyclopentyl, cyclohexyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂C(O)NH₂, —CH₂C(O)NHMe, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-(trifluoromethyl)phenyl, 2-methylphenyl, 4-methylphenyl, 4-(2-hydroxyethylamino)phenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1-(difluoromethyl)-3,5-dimethylpyrazol-4-yl, 1,3-dimethylpyrazol-4-yl, pyridin-4-yl, pyridin-2-yl, 5-fluoropyridin-2-yl, 5-fluoropyridin-3-yl, thiazol-2-yl, 4-methylthiazol-2-yl, 4,5-dimethylthiazol-2-yl, 2,4-dimethylthiazol-5-yl, 5-methyl-1,3,4-thiadiazol-2-yl, terahydrofuran-3-yl and terahydropyran-4-yl;
R² is

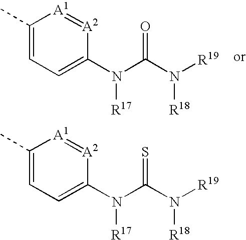

wherein A¹ and A² are CH;
   R¹⁷ is hydrogen;
   R¹⁸ is hydrogen; and
   R¹⁹ is is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₂OH, —C(CH₃)₂CH₂OH, —CH₂C(CH₃)₂OH, —CH₂C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂SO₂Me, —CH₂CH(OH)CF₃, —CH₂CH₂CN, —CH₂CN, —CH₂CONMe₂, —CH₂CO₂H, 1-(methyl)cyclopropyl, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl and 1-methylpyrazol-3-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl, ethyl, hydroxymethyl, dimethylcarbamoyl or carbamoyl; or when R³′ is methyl, R³ is methyl.

In a further particular class of compound of formula (IA), (IB) or (IC)

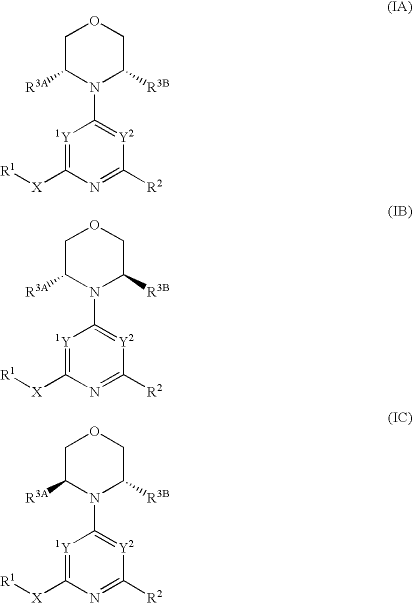

or a pharmaceutically acceptable salt thereof,
X is a —S(O)₂CR⁶R⁷— linker group;
¹Y is CH and Y² is N;
R¹ is a group selected from methyl, ethyl, isopropyl, tert-butyl, cyclopropyl, —CH₂CH₂OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-methylphenyl, 4-methylphenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1,3-dimethylpyrazol-4-yl, pyridin-4-yl, pyridin-2-yl, 5-fluoropyridin-2-yl, thiazol-2-yl, 4-methylthiazol-2-yl, 4,5-dimethylthiazol-2-yl, 2,4-dimethylthiazol-5-yl and 5-methyl-1,3,4-thiadiazol-2-yl;
R² is

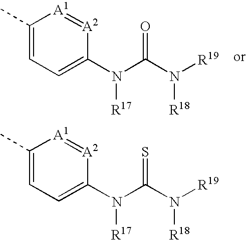

wherein A¹ and A² are CH;
   R¹⁷ is hydrogen;
   R¹⁸ is hydrogen; and
   R¹⁹ is hydrogen or a group selected from methyl, ethyl, propyl, i-propyl, cyclopropyl, cyclobutyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, —CH₂CF₃, —CH₂CHF₂, —CH₂CH₂F, —CH₂CH₂Cl, —CH₂CH₂CN, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 5-methylisoxazol-3-yl, 1-methylpyrazol-4-yl, 5-methylpyrazin-2-yl, 6-methoxypryridin-3-yl, thiazol-2-yl, 1,2,4-thiadiazol-5-yl and 1-methylpyrazol-3-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl or ethyl; or
when R^{3A} is methyl, R^{3B} is methyl.

In a further particular class of compound of formula (IA), (IB) or (IC)

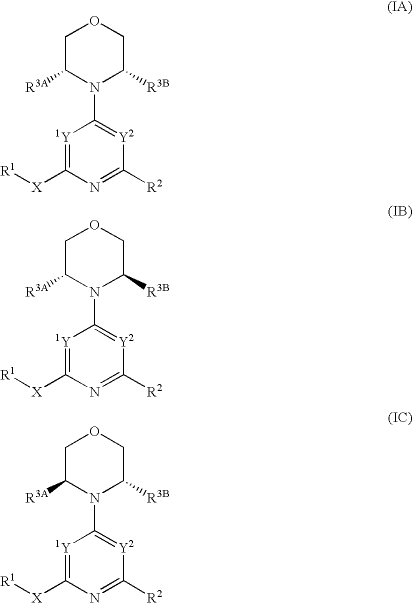

or a pharmaceutically acceptable salt thereof,
X is a —S(O)₂CR⁶R⁷— linker group;
¹Y is CH and Y² is N;
R¹ is a group selected from methyl, ethyl, cyclopropyl, —CH₂CH₂CH₂OH, phenyl, 2-fluorophenyl, 3-fluorophenyl, 4-fluorophenyl, 2-chlorophenyl, 2-methylphenyl, 5-fluoropyridin-2-yl, pyridin-2-yl, thiazol-2-yl and 4-methylthiazol-2-yl;
R² is

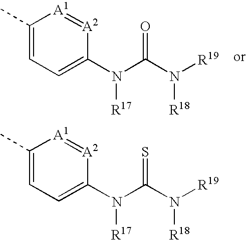

wherein A¹ and A² are CH;
   R¹⁷ is hydrogen;
   R¹⁸ is hydrogen; and
   R¹⁹ is a group selected from methyl, ethyl, cyclopropyl, cyclobutyl, —CH(CH₃)CH₂OH, —CH₂CH₂OH, —CH₂CH₂CH₂OH, —CH₂CHF₂, —CH₂CH₂F, —CH₂CH₂CN, (1R)-2-hydroxy-1-methylethyl, (1S)-2-hydroxy-1-methylethyl, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl or ethyl; or
when R^{3A} is methyl, R^{3B} is methyl.

In a further particular class of compound of formula (IA), (IB) or (IC)

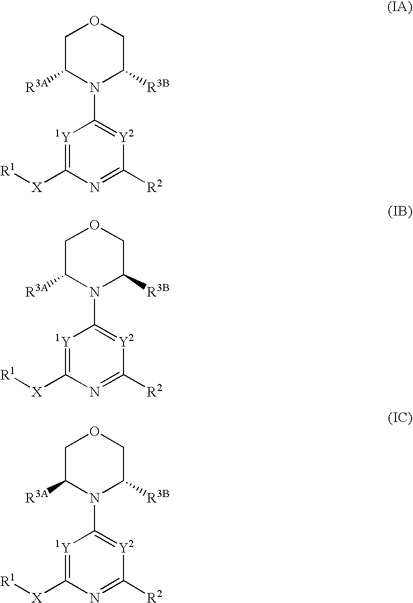

or a pharmaceutically acceptable salt thereof,
X is a —S(O)₂CR⁶R⁷— linker group;
¹Y is CH and Y² is N;
R¹ is a group selected from methyl, ethyl, cyclopropyl, —CH₂CH₂CH₂OH, phenyl, 2-fluorophenyl, 3-fluorophenyl, 4-fluorophenyl, 2-chlorophenyl, 2-methylphenyl, 5-fluoropyridin-2-yl, pyridin-2-yl, thiazol-2-yl and 4-methylthiazol-2-yl;
R² is

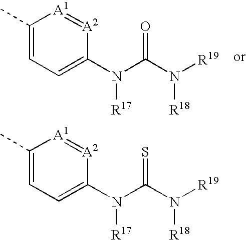

wherein A¹ and A² are CH;
   R¹⁷ is hydrogen;
   R¹⁸ is hydrogen; and
   R¹⁹ is a group selected from methyl, ethyl, cyclopropyl, cyclobutyl, —CH(CH₃)CH₂OH, —CH₂CH₂OH, —CH₂CH₂CH₂OH, —CH₂CHF₂, —CH₂CH₂F, —CH₂CH₂CN, (1R)-2-hydroxy-1-methylethyl, (1S)-2-hydroxy-1-methylethyl, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is methyl or ethyl; or
when R^{3A} is methyl, R^{3B} is methyl.

Another aspect of the invention provides a compound, or a combination of compounds, selected from any one of the Examples or a pharmaceutically acceptable salt thereof.

In another aspect of the invention there is provided a compound, or a combination of compounds, selected from any one of
3-Ethyl-1-[4-[4-[(3S)-3-methylmorpholin-4-yl]-6-(1-methylsulfonylcyclopropyl)pyrimidin-2-yl]phenyl]urea,
3-methyl-1-[4-[4-[(3S)-3-methylmorpholin-4-yl]-6-(1-methylsulfonylcyclopropyl)pyrimidin-2-yl]phenyl]urea,
3-Cyclopropyl-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-[(1-methylpyrazol-4-yl)methyl]urea,
1-[4-[4-[(3S)-3-methylmorpholin-4-yl]-6-(1-methylsulfonylcyclopropyl)pyrimidin-2-yl]phenyl]-3-(1-methylpyrazol-4-yl)urea,
3-cyclobutyl-1-[4-[4-[(3S)-3-methylmorpholin-4-yl]-6-(1-methylsulfonylcyclopropyl)pyrimidin-2-yl]phenyl]urea,
3-(1H-imidazol-2-ylmethyl)-1-[4-[4-[(3S)-3-methylmorpholin-4-yl]-6-(1-methylsulfonylcyclopropyl)pyrimidin-2-yl]phenyl]urea,
3-(2-dimethylaminoethyl)-1-[4-[4-[(3S)-3-methylmorpholin-4-yl]-6-(1-methylsulfonylcyclopropyl)pyrimidin-2-yl]phenyl]urea,
3-(1-hydroxy-2-methyl-propan-2-yl)-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(1,2-oxazol-3-yl)urea,
1-[4-[4-[(3S)-3-methylmorpholin-4-yl]-6-(1-methylsulfonylcyclopropyl)pyrimidin-2-yl]phenyl]-3-phenyl-urea,
3-(2-hydroxyethyl)-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-propan-2-yl-urea,
1-[4-[4-[(3S)-3-methylmorpholin-4-yl]-6-(1-methylsulfonylcyclopropyl)pyrimidin-2-yl]phenyl]-3-propyl-urea,
1-[4-[4-[(3S)-3-methylmorpholin-4-yl]-6-(1-methylsulfonylcyclopropyl)pyrimidin-2-yl]phenyl]-3-(2-methylpropyl)urea,
3-(cyclopropylmethyl)-1-[4-[4-[(3S)-3-methylmorpholin-4-yl]-6-(1-methylsulfonylcyclopropyl)pyrimidin-2-yl]phenyl]urea,
3-(1-hydroxypropan-2-yl)-1-[4-[4-[(3S)-3-methylmorpholin-4-yl]-6-(1-methylsulfonylcyclopropyl)pyrimidin-2-yl]phenyl]urea,
3-(6-methoxypyridin-3-yl)-1-[4-[4-[(3S)-3-methylmorpholin-4-yl]-6-(1-methylsulfonylcyclopropyl)pyrimidin-2-yl]phenyl]urea,
3-(4-fluorophenyl)-1-[4-[4-[(3S)-3-methylmorpholin-4-yl]-6-(1-methylsulfonylcyclopropyl)pyrimidin-2-yl]phenyl]urea,
3-(3,4-difluorophenyl)-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(4-methylphenyl)urea,
3-(4-chlorophenyl)-1-[4-[4-[(3S)-3-methylmorpholin-4-yl]-6-(1-methylsulfonylcyclopropyl)pyrimidin-2-yl]phenyl]urea,
3-(4-methoxyphenyl)-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(5-methyl-1,2-oxazol-3-yl)urea,
3-(5-fluoropyridin-2-yl)-1-[4-[4-[(3S)-3-methylmorpholin-4-yl]-6-(1-methylsulfonylcyclopropyl)pyrimidin-2-yl]phenyl]urea,
3-(3-hydroxy-2,2-dimethylpropyl)-1-[4-[4-[(3S)-3-methylmorpholin-4-yl]-6-(1-methylsulfonylcyclopropyl)pyrimidin-2-yl]phenyl]urea,
3-(2-cyanoethyl)-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(2-pyrrolidin-1-ylethyl)urea,
1-[4-[4-[(3S)-3-methylmorpholin-4-yl]-6-(1-methylsulfonylcyclopropyl)pyrimidin-2-yl]phenyl]-3-(3,3,3-trifluoro-2-hydroxypropyl)urea,
3-(2-hydroxy-2-methylpropyl)-1-[4-[4-[(3S)-3-methylmorpholin-4-yl]-6-(1-methylsulfonylcyclopropyl)pyrimidin-2-yl]phenyl]urea,
3-[1-(hydroxymethyl)cyclopropyl]-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(oxetan-3-yl)urea,
1-[4-[4-[(3S)-3-methylmorpholin-4-yl]-6-(1-methylsulfonylcyclopropyl)pyrimidin-2-yl]phenyl]-3-(1-methylpyrazol-3-yl)urea,
1-[4-[4-[(3S)-3-methylmorpholin-4-yl]-6-(1-methylsulfonylcyclopropyl)pyrimidin-2-yl]phenyl]-3-(1,2,4-thiadiazol-5-yl)urea,
3-(cyanomethyl)-1-[4-[4-[(3S)-3-methylmorpholin-4-yl]-6-(1-methylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(2H-1,2,4-triazol-3-ylmethyl)urea,
3-cyclopropyl-1-[4-[4-(1-cyclopropylsulfonylcyclopropyl)-6-[(3S)-3-methylmorpholin-4-yl]pyrimidin-2-yl]phenyl]urea,
3-cyclobutyl-1-[4-[4-(1-cyclopropylsulfonylcyclopropyl)-6-[(3S)-3-methylmorpholin-4-yl]pyrimidin-2-yl]phenyl]urea,
1-[4-[4-(1-cyclopropylsulfonylcyclopropyl)-6-[(3S)-3-methylmorpholin-4-yl]pyrimidin-2-yl]phenyl]-3-pyridin-2-yl-urea,
1-[4-[4-(1-cyclopropylsulfonylcyclopropyl)-6-[(3S)-3-methylmorpholin-4-yl]pyrimidin-2-yl]phenyl]-3-(2-methylpropyl)urea,
1-[4-[4-(1-cyclopropylsulfonylcyclopropyl)-6-[(3S)-3-methylmorpholin-4-yl]pyrimidin-2-yl]phenyl]-3-propan-2-yl-urea,
3-[4-[4-(1-cyclopropylsulfonylcyclopropyl)-6-[(3S)-3-methylmorpholin-4-yl]pyrimidin-2-yl]phenyl]-1-ethyl-urea,
1-[4-[4-(1-cyclopropylsulfonylcyclopropyl)-6-[(3S)-3-methylmorpholin-4-yl]pyrimidin-2-yl]phenyl]-3-(2-dimethylaminoethyl)urea,
1-[4-[4-(1-cyclopropylsulfonylcyclopropyl)-6-[(3S)-3-methylmorpholin-4-yl]pyrimidin-2-yl]phenyl]-3-(2-hydroxyethyl)urea,
3-[4-[4-(1-cyclopropylsulfonylcyclopropyl)-6-[(3S)-3-methylmorpholin-4-yl]pyrimidin-2-yl]phenyl]-1-propyl-urea,
1-[4-[4-(1-cyclopropylsulfonylcyclopropyl)-6-[(3S)-3-methylmorpholin-4-yl]pyrimidin-2-yl]phenyl]-3-methyl-urea,
1-[4-[4-(1-cyclopropylsulfonylcyclopropyl)-6-[(3S)-3-methylmorpholin-4-yl]pyrimidin-2-yl]phenyl]-3-[4-(trifluoromethyl)phenyl]urea,
1-[4-[4-(1-cyclopropylsulfonylcyclopropyl)-6-[(3S)-3-methylmorpholin-4-yl]pyrimidin-2-yl]phenyl]-3-(1-hydroxy-2-methyl-propan-2-yl)urea,
1-[4-[4-(1-cyclopropylsulfonylcyclopropyl)-6-[(3S)-3-methylmorpholin-4-yl]pyrimidin-2-yl]phenyl]-3-(3-hydroxypropyl)urea,
1-[4-[4-(1-cyclopropylsulfonylcyclopropyl)-6-[(3S)-3-methylmorpholin-4-yl]pyrimidin-2-yl]phenyl]-3-(1-methylpyrazol-4-yl)urea,
1-[4-[4-(1-cyclopropylsulfonylcyclopropyl)-6-[(3S)-3-methylmorpholin-4-yl]pyrimidin-2-yl]phenyl]-3-(3-hydroxy-2,2-dimethylpropyl)urea,
3-(2-cyanoethyl)-1-[4-[4-(1-cyclopropylsulfonylcyclopropyl)-6-[(3S)-3-methylmorpholin-4-yl]pyrimidin-2-yl]phenyl]urea,
1-[4-[4-(1-cyclopropylsulfonylcyclopropyl)-6-[(3S)-3-methylmorpholin-4-yl]pyrimidin-2-yl]phenyl]-3-(2-pyrrolidin-1-ylethyl)urea,
1-[4-[4-(1-cyclopropylsulfonylcyclopropyl)-6-[(3S)-3-methylmorpholin-4-yl]pyrimidin-2-yl]phenyl]-3-(3,3,3-trifluoro-2-hydroxypropyl)urea,
1-[4-[4-(1-cyclopropylsulfonylcyclopropyl)-6-[(3S)-3-methylmorpholin-4-yl]pyrimidin-2-yl]phenyl]-3-(2-hydroxy-2-methylpropyl)urea,
1-[4-[4-(1-cyclopropylsulfonylcyclopropyl)-6-[(3S)-3-methylmorpholin-4-yl]pyrimidin-2-yl]phenyl]-3-[1-(hydroxymethyl)cyclopropyl]urea,
1-[4-[4-(1-cyclopropylsulfonylcyclopropyl)-6-[(3S)-3-methylmorpholin-4-yl]pyrimidin-2-yl]phenyl]-3-(oxetan-3-yl)urea,
1-[4-[4-(1-cyclopropylsulfonylcyclopropyl)-6-[(3S)-3-methylmorpholin-4-yl]pyrimidin-2-yl]phenyl]-3-(1-methylpyrazol-3-yl)urea,
1-[4-[4-(1-cyclopropylsulfonylcyclopropyl)-6-[(3S)-3-methylmorpholin-4-yl]pyrimidin-2-yl]phenyl]-3-(1,2,4-thiadiazol-5-yl)urea,
3-methyl-1-[4-[4-[(3S)-3-methylmorpholin-4-yl]-6-(1-methylsulfonylcyclopentyl)pyrimidin-2-yl]phenyl]urea,
3-ethyl-1-[4-[4-[(3S)-3-methylmorpholin-4-yl]-6-(1-methylsulfonylcyclopentyl)pyrimidin-2-yl]phenyl]urea,
3-cyclopropyl-1-[4-[4-[(3S)-3-methylmorpholin-4-yl]-6-(1-methylsulfonylcyclopentyl)pyrimidin-2-yl]phenyl]urea,
1-[4-[4-[(3S)-3-methylmorpholin-4-yl]-6-(1-methylsulfonylcyclopentyl)pyrimidin-2-yl]phenyl]-3-propan-2-yl-urea,
3-cyclobutyl-1-[4-[4-[(3S)-3-methylmorpholin-4-yl]-6-(1-methylsulfonylcyclopentyl)pyrimidin-2-yl]phenyl]urea,
3-(2-hydroxyethyl)-1-[4-[4-[(3S)-3-methylmorpholin-4-yl]-6-(1-methylsulfonylcyclopentyl)pyrimidin-2-yl]phenyl]urea,
3-(1-hydroxy-2-methyl-propan-2-yl)-1-[4-[4-[(3S)-3-methylmorpholin-4-yl]-6-(1-methylsulfonylcyclopentyl)pyrimidin-2-yl]phenyl]urea,
3-(2-dimethylaminoethyl)-1-[4-[4-[(3S)-3-methylmorpholin-4-yl]-6-(1-methylsulfonylcyclopentyl)pyrimidin-2-yl]phenyl]urea,
1-[4-[4-[(3S)-3-methylmorpholin-4-yl]-6-(1-methylsulfonylcyclopentyl)pyrimidin-2-yl]phenyl]-3-propyl-urea,
1-[4-[4-[(3S)-3-methylmorpholin-4-yl]-6-(1-methylsulfonylcyclopentyl)pyrimidin-2-yl]phenyl]-3-(2-methylpropyl)urea,
3-(3-hydroxypropyl)-1-[4-[4-[(3S)-3-methylmorpholin-4-yl]-6-(1-methylsulfonylcyclopentyl)pyrimidin-2-yl]phenyl]urea,
1-[4-[4-[(3S)-3-methylmorpholin-4-yl]-6-(1-methylsulfonylcyclopentyl)pyrimidin-2-yl]phenyl]-3-[4-(trifluoromethyl)phenyl]urea,
1-[4-[4-[(3S)-3-methylmorpholin-4-yl]-6-(1-methylsulfonylcyclopentyl)pyrimidin-2-yl]phenyl]-3-pyridin-2-yl-urea,
1-[4-[4-[(3S)-3-methylmorpholin-4-yl]-6-(1-methylsulfonylcyclopentyl)pyrimidin-2-yl]phenyl]-3-(1-methylpyrazol-4-yl)urea,
3-(1H-imidazol-2-ylmethyl)-1-[4-[4-[(3S)-3-methylmorpholin-4-yl]-6-(1-methylsulfonylcyclopentyl)pyrimidin-2-yl]phenyl]urea,
3-methyl-1-[4-[4-[(3S)-3-methylmorpholin-4-yl]-6-(1-methylsulfonylcyclobutyl)pyrimidin-2-yl]phenyl]urea,
3-ethyl-1-[4-[4-[(3S)-3-methylmorpholin-4-yl]-6-(1-methylsulfonylcyclobutyl)pyrimidin-2-yl]phenyl]urea,
3-cyclopropyl-1-[4-[4-[(3S)-3-methylmorpholin-4-yl]-6-(1-methylsulfonylcyclobutyl)pyrimidin-2-yl]phenyl]urea,
1-[4-[4-[(3S)-3-methylmorpholin-4-yl]-6-(1-methylsulfonylcyclobutyl)pyrimidin-2-yl]phenyl]-3-propan-2-yl-urea,
3-cyclobutyl-1-[4-[4-[(3S)-3-methylmorpholin-4-yl]-6-(1-methylsulfonylcyclobutyl)pyrimidin-2-yl]phenyl]urea,
3-(2-hydroxyethyl)-1-[4-[4-[(3S)-3-methylmorpholin-4-yl]-6-(1-methylsulfonylcyclobutyl)pyrimidin-2-yl]phenyl]urea,
3-(1-hydroxy-2-methyl-propan-2-yl)-1-[4-[4-[(3S)-3-methylmorpholin-4-yl]-6-(1-methylsulfonylcyclobutyl)pyrimidin-2-yl]phenyl]urea,
3-(2-dimethylaminoethyl)-1-[4-[4-[(3S)-3-methylmorpholin-4-yl]-6-(1-methylsulfonylcyclobutyl)pyrimidin-2-yl]phenyl]urea,
1-[4-[4-[(3S)-3-methylmorpholin-4-yl]-6-(1-methylsulfonylcyclobutyl)pyrimidin-2-yl]phenyl]-3-propyl-urea,
1-[4-[4-[(3S)-3-methylmorpholin-4-yl]-6-(1-methylsulfonylcyclobutyl)pyrimidin-2-yl]phenyl]-3-(2-methylpropyl)urea,
3-(3-hydroxypropyl)-1-[4-[4-[(3S)-3-methylmorpholin-4-yl]-6-(1-methylsulfonylcyclobutyl)pyrimidin-2-yl]phenyl]urea,
1-[4-[4-[(3S)-3-methylmorpholin-4-yl]-6-(1-methylsulfonylcyclobutyl)pyrimidin-2-yl]phenyl]-3-[4-(trifluoromethyl)phenyl]urea,
1-[4-[4-[(3S)-3-methylmorpholin-4-yl]-6-(1-methylsulfonylcyclobutyl)pyrimidin-2-yl]phenyl]-3-pyridin-2-yl-urea,
1-[4-[4-[(3S)-3-methylmorpholin-4-yl]-6-(1-methylsulfonylcyclobutyl)pyrimidin-2-yl]phenyl]-3-(1-methylpyrazol-4-yl)urea,
3-(1H-imidazol-2-ylmethyl)-1-[4-[4-[(3S)-3-methylmorpholin-4-yl]-6-(1-methylsulfonylcyclobutyl)pyrimidin-2-yl]phenyl]urea,
3-cyclobutyl-1-[4-[4-[(3S)-3-methylmorpholin-4-yl]-6-(1-pyridin-4-ylsulfonylcyclopropyl)pyrimidin-2-yl]phenyl]urea,
1-[4-[4-[(3S)-3-methylmorpholin-4-yl]-6-(1-pyridin-4-ylsulfonylcyclopropyl)pyrimidin-2-yl]phenyl]-3-pyridin-2-yl-urea,
1-[4-[4-[(3S)-3-methylmorpholin-4-yl]-6-(1-pyridin-4-ylsulfonylcyclopropyl)pyrimidin-2-yl]phenyl]-3-(2-methylpropyl)urea,
1-[4-[4-[(3S)-3-methylmorpholin-4-yl]-6-(1-pyridin-4-ylsulfonylcyclopropyl)pyrimidin-2-yl]phenyl]-3-propan-2-yl-urea,
3-ethyl-1-[4-[4-[(3S)-3-methylmorpholin-4-yl]-6-(1-pyridin-4-ylsulfonylcyclopropyl)pyrimidin-2-yl]phenyl]urea,
3-(2-dimethylaminoethyl)-1-[4-[4-[(3S)-3-methylmorpholin-4-yl]-6-(1-pyridin-4-ylsulfonylcyclopropyl)pyrimidin-2-yl]phenyl]urea,
3-(2-hydroxyethyl)-1-[4-[4-[(3S)-3-methylmorpholin-4-yl]-6-(1-pyridin-4-ylsulfonylcyclopropyl)pyrimidin-2-yl]phenyl]urea,
1-[4-[4-[(3S)-3-methylmorpholin-4-yl]-6-(1-pyridin-4-ylsulfonylcyclopropyl)pyrimidin-2-yl]phenyl]-3-propyl-urea,
3-methyl-1-[4-[4-[(3S)-3-methylmorpholin-4-yl]-6-(1-pyridin-4-ylsulfonylcyclopropyl)pyrimidin-2-yl]phenyl]urea,
1-[4-[4-[(3S)-3-methylmorpholin-4-yl]-6-(1-pyridin-4-ylsulfonylcyclopropyl)pyrimidin-2-yl]phenyl]-3-[4-(trifluoromethyl)phenyl]urea,
3-(1-hydroxy-2-methyl-propan-2-yl)-1-[4-[4-[(3S)-3-methylmorpholin-4-yl]-6-(1-pyridin-4-ylsulfonylcyclopropyl)pyrimidin-2-yl]phenyl]urea,
3-(3-hydroxypropyl)-1-[4-[4-[(3S)-3-methylmorpholin-4-yl]-6-(1-pyridin-4-ylsulfonylcyclopropyl)pyrimidin-2-yl]phenyl]urea,
1-[4-[4-[(3S)-3-methylmorpholin-4-yl]-6-(1-pyridin-4-ylsulfonylcyclopropyl)pyrimidin-2-yl]phenyl]-3-(1-methylpyrazol-4-yl)urea,
3-cyclopropyl-1-[4-[4-[(3S)-3-methylmorpholin-4-yl]-6-(1-pyridin-4-ylsulfonylcyclopropyl)pyrimidin-2-yl]phenyl]urea,
3-methyl-1-[4-[4-[(3S)-3-methylmorpholin-4-yl]-6-(1-propan-2-ylsulfonylcyclopropyl)pyrimidin-2-yl]phenyl]urea,
1-ethyl-3-[4-[4-[(3S)-3-methylmorpholin-4-yl]-6-(1-propan-2-ylsulfonylcyclopropyl)pyrimidin-2-yl]phenyl]urea,
1-[4-[4-[(3S)-3-methylmorpholin-4-yl]-6-(1-propan-2-ylsulfonylcyclopropyl)pyrimidin-2-yl]phenyl]-3-propan-2-yl-urea,
3-cyclobutyl-1-[4-[4-[(3S)-3-methylmorpholin-4-yl]-6-(1-propan-2-ylsulfonylcyclopropyl)pyrimidin-2-yl]phenyl]urea,
3-cyclopropyl-1-[4-[4-[(3S)-3-methylmorpholin-4-yl]-6-(1-propan-2-ylsulfonylcyclopropyl)pyrimidin-2-yl]phenyl]urea,
3-(2-hydroxyethyl)-1-[4-[4-[(3S)-3-methylmorpholin-4-yl]-6-(1-propan-2-ylsulfonylcyclopropyl)pyrimidin-2-yl]phenyl]urea,
3-(1-hydroxy-2-methyl-propan-2-yl)-1-[4-[4-[(3S)-3-methylmorpholin-4-yl]-6-(1-propan-2-ylsulfonylcyclopropyl)pyrimidin-2-yl]phenyl]urea,
3-(2-dimethylaminoethyl)-1-[4-[4-[(3S)-3-methylmorpholin-4-yl]-6-(1-propan-2-ylsulfonylcyclopropyl)pyrimidin-2-yl]phenyl]urea,
3-[4-[4-[(3S)-3-methylmorpholin-4-yl]-6-(1-propan-2-ylsulfonylcyclopropyl)pyrimidin-2-yl]phenyl]-1-propyl-urea,
1-[4-[4-[(3S)-3-methylmorpholin-4-yl]-6-(1-propan-2-ylsulfonylcyclopropyl)pyrimidin-2-yl]phenyl]-3-(2-methylpropyl)urea,
3-(3-hydroxypropyl)-1-[4-[4-[(3S)-3-methylmorpholin-4-yl]-6-(1-propan-2-ylsulfonylcyclopropyl)pyrimidin-2-yl]phenyl]urea,
1-[4-[4-[(3S)-3-methylmorpholin-4-yl]-6-(1-propan-2-ylsulfonylcyclopropyl)pyrimidin-2-yl]phenyl]-3-[4-(trifluoromethyl)phenyl]urea,
1-[4-[4-[(3S)-3-methylmorpholin-4-yl]-6-(1-propan-2-ylsulfonylcyclopropyl)pyrimidin-2-yl]phenyl]-3-pyridin-2-yl-urea,
1-[4-[4-[(3S)-3-methylmorpholin-4-yl]-6-(1-propan-2-ylsulfonylcyclopropyl)pyrimidin-2-yl]phenyl]-3-(1-methylpyrazol-4-yl)urea,
1-[4-[4-[1-(4-fluorophenyl)sulfonylcyclopropyl]-6-[(3S)-3-methylmorpholin-4-yl]pyrimidin-2-yl]phenyl]-3-methyl-urea,
1-ethyl-3-[4-[4-[1-(4-fluorophenyl)sulfonylcyclopropyl]-6-[(3S)-3-methylmorpholin-4-yl]pyrimidin-2-yl]phenyl]urea,
3-cyclopropyl-1-[4-[4-[1-(4-fluorophenyl)sulfonylcyclopropyl]-6-[(3S)-3-methylmorpholin-4-yl]pyrimidin-2-yl]phenyl]urea,
1-[4-[4-[1-(4-fluorophenyl)sulfonylcyclopropyl]-6-[(3S)-3-methylmorpholin-4-yl]pyrimidin-2-yl]phenyl]-3-propan-2-yl-urea,
3-cyclobutyl-1-[4-[4-[1-(4-fluorophenyl)sulfonylcyclopropyl]-6-[(3S)-3-methylmorpholin-4-yl]pyrimidin-2-yl]phenyl]urea,
1-[4-[4-[1-(4-fluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(4-fluorophenyl)sulfonylcyclopropyl]-6-[(3S)-3-methylmorpholin-4-yl]pyrimidin-2-yl]phenyl]-3-(1-hydroxy-2-methyl-propan-2-yl)urea,
3-(2-dimethylaminoethyl)-1-[4-[4-[1-(4-fluorophenyl)sulfonylcyclopropyl]-6-[(3S)-3-methylmorpholin-4-yl]pyrimidin-2-yl]phenyl]urea,
3-[4-[4-[1-(4-fluorophenyl)sulfonylcyclopropyl]-6-[(3S)-3-methylmorpholin-4-yl]pyrimidin-2-yl]phenyl]-1-propyl-urea,
1-[4-[4-[1-(4-fluorophenyl)sulfonylcyclopropyl]-6-[(3S)-3-methylmorpholin-4-yl]pyrimidin-2-yl]phenyl]-3-(2-methylpropyl)urea,
1-[4-[4-[1-(4-fluorophenyl)sulfonylcyclopropyl]-6-[(3S)-3-methylmorpholin-4-yl]pyrimidin-2-yl]phenyl]-3-(3-hydroxypropyl)urea,
1-[4-[4-[1-(4-fluorophenyl)sulfonylcyclopropyl]-6-[(3S)-3-methylmorpholin-4-yl]pyrimidin-2-yl]phenyl]-3-[4-(trifluoromethyl)phenyl]urea,
1-[4-[4-[1-(4-fluorophenyl)sulfonylcyclopropyl]-6-[(3S)-3-methylmorpholin-4-yl]pyrimidin-2-yl]phenyl]-3-pyridin-2-yl-urea,
1-[4-[4-[1-(4-fluorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-(1-cyclopentylsulfonylcyclopropyl)-6-[(3S)-3-methylmorpholin-4-yl]pyrimidin-2-yl]phenyl]-3-cyclopropyl-urea,
3-cyclobutyl-1-[4-[4-(1-cyclopentylsulfonylcyclopropyl)-6-[(3S)-3-methylmorpholin-4-yl]pyrimidin-2-yl]phenyl]urea,
1-[4-[4-(1-cyclopentylsulfonylcyclopropyl)-6-[(3S)-3-methylmorpholin-4-yl]pyrimidin-2-yl]phenyl]-3-pyridin-2-yl-urea,
1-[4-[4-(1-cyclopentylsulfonylcyclopropyl)-6-[(3S)-3-methylmorpholin-4-yl]pyrimidin-2-yl]phenyl]-3-(2-methylpropyl)urea,
1-[4-[4-(1-cyclopentylsulfonylcyclopropyl)-6-[(3S)-3-methylmorpholin-4-yl]pyrimidin-2-yl]phenyl]-3-propan-2-yl-urea,
3-[4-[4-(1-cyclopentylsulfonylcyclopropyl)-6-[(3S)-3-methylmorpholin-4-yl]pyrimidin-2-yl]phenyl]-1-ethyl-urea,
1-[4-[4-(1-cyclopentylsulfonylcyclopropyl)-6-[(3S)-3-methylmorpholin-4-yl]pyrimidin-2-yl]phenyl]-3-(2-dimethylaminoethyl)urea,
1-[4-[4-(1-cyclopentylsulfonylcyclopropyl)-6-[(3S)-3-methylmorpholin-4-yl]pyrimidin-2-yl]phenyl]-3-(2-hydroxyethyl)urea,
3-[4-[4-(1-cyclopentylsulfonylcyclopropyl)-6-[(3S)-3-methylmorpholin-4-yl]pyrimidin-2-yl]phenyl]-1-propyl-urea,
1-[4-[4-(1-cyclopentylsulfonylcyclopropyl)-6-[(3S)-3-methylmorpholin-4-yl]pyrimidin-2-yl]phenyl]-3-methyl-urea,
1-[4-[4-(1-cyclopentylsulfonylcyclopropyl)-6-[(3S)-3-methylmorpholin-4-yl]pyrimidin-2-yl]phenyl]-3-[4-(trifluoromethyl)phenyl]urea,
1-[4-[4-(1-cyclopentylsulfonylcyclopropyl)-6-[(3S)-3-methylmorpholin-4-yl]pyrimidin-2-yl]phenyl]-3-(1-hydroxy-2-methyl-propan-2-yl)urea,
1-[4-[4-(1-cyclopentylsulfonylcyclopropyl)-6-[(3S)-3-methylmorpholin-4-yl]pyrimidin-2-yl]phenyl]-3-(3-hydroxypropyl)urea,
1-[4-[4-(1-cyclopentylsulfonylcyclopropyl)-6-[(3S)-3-methylmorpholin-4-yl]pyrimidin-2-yl]phenyl]-3-(1-methylpyrazol-4-yl)urea,
3-cyclopropyl-1-[4-[4-[(3S)-3-methylmorpholin-4-yl]-6-[1-[2-(trifluoromethyl)phenyl]sulfonylcyclopropyl]pyrimidin-2-yl]phenyl]urea,
3-cyclobutyl-1-[4-[4-[(3S)-3-methylmorpholin-4-yl]-6-[1-[2-(trifluoromethyl)phenyl]sulfonylcyclopropyl]pyrimidin-2-yl]phenyl]urea,
1-[4-[4-[(3S)-3-methylmorpholin-4-yl]-6-[1-[2-(trifluoromethyl)phenyl]sulfonylcyclopropyl]pyrimidin-2-yl]phenyl]-3-pyridin-2-yl-urea,
1-[4-[4-[(3S)-3-methylmorpholin-4-yl]-6-[1-[2-(trifluoromethyl)phenyl]sulfonylcyclopropyl]pyrimidin-2-yl]phenyl]-3-(2-methylpropyl)urea,
1-[4-[4-[(3S)-3-methylmorpholin-4-yl]-6-[1-[2-(trifluoromethyl)phenyl]sulfonylcyclopropyl]pyrimidin-2-yl]phenyl]-3-propan-2-yl-urea,
1-ethyl-3-[4-[4-[(3S)-3-methylmorpholin-4-yl]-6-[1-[2-(trifluoromethyl)phenyl]sulfonylcyclopropyl]pyrimidin-2-yl]phenyl]urea,
3-(2-dimethylaminoethyl)-1-[4-[4-[(3S)-3-methylmorpholin-4-yl]-6-[1-[2-(trifluoromethyl)phenyl]sulfonylcyclopropyl]pyrimidin-2-yl]phenyl]urea,
3-(2-hydroxyethyl)-1-[4-[4-[(3S)-3-methylmorpholin-4-yl]-6-[1-[2-(trifluoromethyl)phenyl]sulfonylcyclopropyl]pyrimidin-2-yl]phenyl]urea,
3-[4-[4-[(3S)-3-methylmorpholin-4-yl]-6-[1-[2-(trifluoromethyl)phenyl]sulfonylcyclopropyl]pyrimidin-2-yl]phenyl]-1-propyl-urea,
3-methyl-1-[4-[4-[(3S)-3-methylmorpholin-4-yl]-6-[1-[2-(trifluoromethyl)phenyl]sulfonylcyclopropyl]pyrimidin-2-yl]phenyl]urea,
1-[4-[4-[(3S)-3-methylmorpholin-4-yl]-6-[1-[2-(trifluoromethyl)phenyl]sulfonylcyclopropyl]pyrimidin-2-yl]phenyl]-3-[4-(trifluoromethyl)phenyl]urea,
3-(1-hydroxy-2-methyl-propan-2-yl)-1-[4-[4-[(3S)-3-methylmorpholin-4-yl]-6-[1-[2-(trifluoromethyl)phenyl]sulfonylcyclopropyl]pyrimidin-2-yl]phenyl]urea,
3-(3-hydroxypropyl)-1-[4-[4-[(3S)-3-methylmorpholin-4-yl]-6-[1-[2-(trifluoromethyl)phenyl]sulfonylcyclopropyl]pyrimidin-2-yl]phenyl]urea,
1-[4-[4-[(3S)-3-methylmorpholin-4-yl]-6-[1-[2-(trifluoromethyl)phenyl]sulfonylcyclopropyl]pyrimidin-2-yl]phenyl]-3-(1-methylpyrazol-4-yl)urea,
3-cyclopropyl-1-[4-[4-(1-ethylsulfonylcyclopropyl)-6-[(3S)-3-methylmorpholin-4-yl]pyrimidin-2-yl]phenyl]urea,
1-[4-[4-(1-ethylsulfonylcyclopropyl)-6-[(3S)-3-methylmorpholin-4-yl]pyrimidin-2-yl]phenyl]-3-methyl-urea,
1-ethyl-3-[4-[4-(1-ethylsulfonylcyclopropyl)-6-[(3S)-3-methylmorpholin-4-yl]pyrimidin-2-yl]phenyl]urea,
1-[4-[4-(1-ethylsulfonylcyclopropyl)-6-[(3S)-3-methylmorpholin-4-yl]pyrimidin-2-yl]phenyl]-3-propan-2-yl-urea,
3-cyclobutyl-1-[4-[4-(1-ethylsulfonylcyclopropyl)-6-[(3S)-3-methylmorpholin-4-yl]pyrimidin-2-yl]phenyl]urea,
1-[4-[4-(1-ethylsulfonylcyclopropyl)-6-[(3S)-3-methylmorpholin-4-yl]pyrimidin-2-yl]phenyl]-3-(2-hydroxyethyl)urea,
1-[4-[4-(1-ethylsulfonylcyclopropyl)-6-[(3S)-3-methylmorpholin-4-yl]pyrimidin-2-yl]phenyl]-3-(1-hydroxy-2-methyl-propan-2-yl)urea,
3-(2-dimethylaminoethyl)-1-[4-[4-(1-ethylsulfonylcyclopropyl)-6-[(3S)-3-methylmorpholin-4-yl]pyrimidin-2-yl]phenyl]urea,
3-[4-[4-(1-ethylsulfonylcyclopropyl)-6-[(3S)-3-methylmorpholin-4-yl]pyrimidin-2-yl]phenyl]-1-propyl-urea,
1-[4-[4-(1-ethylsulfonylcyclopropyl)-6-[(3S)-3-methylmorpholin-4-yl]pyrimidin-2-yl]phenyl]-3-(2-methylpropyl)urea,
1-[4-[4-(1-ethylsulfonylcyclopropyl)-6-[(3S)-3-methylmorpholin-4-yl]pyrimidin-2-yl]phenyl]-3-(3-hydroxypropyl)urea,
1-[4-[4-(1-ethylsulfonylcyclopropyl)-6-[(3S)-3-methylmorpholin-4-yl]pyrimidin-2-yl]phenyl]-3-[4-(trifluoromethyl)phenyl]urea,
1-[4-[4-(1-ethylsulfonylcyclopropyl)-6-[(3S)-3-methylmorpholin-4-yl]pyrimidin-2-yl]phenyl]-3-pyridin-2-yl-urea,
1-[4-[4-(1-ethylsulfonylcyclopropyl)-6-[(3S)-3-methylmorpholin-4-yl]pyrimidin-2-yl]phenyl]-3-(1-methylpyrazol-4-yl)urea,
3-cyclopropyl-1-[4-[4-(1-methylsulfonylcyclopropyl)-6-morpholin-4-ylpyrimidin-2-yl]phenyl]urea,
3-methyl-1-[4-[4-(1-methylsulfonylcyclopropyl)-6-morpholin-4-ylpyrimidin-2-yl]phenyl]urea,
3-ethyl-1-[4-[4-(1-methylsulfonylcyclopropyl)-6-morpholin-4-ylpyrimidin-2-yl]phenyl]urea,
3-cyclobutyl-1-[4-[4-(1-methylsulfonylcyclopropyl)-6-morpholin-4-ylpyrimidin-2-yl]phenyl]urea,
3-(2-hydroxyethyl)-1-[4-[4-(1-methylsulfonylcyclopropyl)-6-morpholin-4-ylpyrimidin-2-yl]phenyl]urea,
3-(1-hydroxy-2-methylpropan-2-yl)-1-[4-[4-(1-methylsulfonylcyclopropyl)-6-morpholin-4-ylpyrimidin-2-yl]phenyl]urea,
3-(2-dimethylaminoethyl)-1-[4-[4-(1-methylsulfonylcyclopropyl)-6-morpholin-4-ylpyrimidin-2-yl]phenyl]urea,
1-[4-[4-(1-methylsulfonylcyclopropyl)-6-morpholin-4-ylpyrimidin-2-yl]phenyl]-3-propylurea,
3-(3-hydroxypropyl)-1-[4-[4-(1-methylsulfonylcyclopropyl)-6-morpholin-4-ylpyrimidin-2-yl]phenyl]urea,
3-(1-methylpyrazol-4-yl)-1-[4-[4-(1-methylsulfonylcyclopropyl)-6-morpholin-4-ylpyrimidin-2-yl]phenyl]urea,
3-cyclopropyl-1-[4-[4-(1-methylsulfonylcyclopentyl)-6-morpholin-4-ylpyrimidin-2-yl]phenyl]urea,
3-methyl-1-[4-[4-(1-methylsulfonylcyclopentyl)-6-morpholin-4-ylpyrimidin-2-yl]phenyl]urea,
3-(2-hydroxyethyl)-1-[4-[4-(1-methylsulfonylcyclopentyl)-6-morpholin-4-ylpyrimidin-2-yl]phenyl]urea,
3-(1-hydroxy-2-methylpropan-2-yl)-1-[4-[4-(1-methylsulfonylcyclopentyl)-6-morpholin-4-ylpyrimidin-2-yl]phenyl]urea,
3-(2-dimethylaminoethyl)-1-[4-[4-(1-methylsulfonylcyclopentyl)-6-morpholin-4-ylpyrimidin-2-yl]phenyl]urea,
3-(3-hydroxypropyl)-1-[4-[4-(1-methylsulfonylcyclopentyl)-6-morpholin-4-ylpyrimidin-2-yl]phenyl]urea,
3-cyclopropyl-1-[4-[4-(1-methylsulfonylcyclobutyl)-6-morpholin-4-ylpyrimidin-2-yl]phenyl]urea,
3-methyl-1-[4-[4-(1-methylsulfonylcyclobutyl)-6-morpholin-4-ylpyrimidin-2-yl]phenyl]urea,
3-(2-hydroxyethyl)-1-[4-[4-(1-methylsulfonylcyclobutyl)-6-morpholin-4-ylpyrimidin-2-yl]phenyl]urea,
3-(2-dimethylaminoethyl)-1-[4-[4-(1-methylsulfonylcyclobutyl)-6-morpholin-4-ylpyrimidin-2-yl]phenyl]urea,
3-(3-hydroxypropyl)-1-[4-[4-(1-methylsulfonylcyclobutyl)-6-morpholin-4-ylpyrimidin-2-yl]phenyl]urea,
3-cyclopropyl-1-[4-[4-(1-cyclopropylsulfonylcyclopropyl)-6-morpholin-4-ylpyrimidin-2-yl]phenyl]urea,
1-[4-[4-(1-cyclopropylsulfonylcyclopropyl)-6-morpholin-4-ylpyrimidin-2-yl]phenyl]-3-methylurea,
1-[4-[4-(1-cyclopropylsulfonylcyclopropyl)-6-morpholin-4-ylpyrimidin-2-yl]phenyl]-3-ethylurea,
3-cyclobutyl-1-[4-[4-(1-cyclopropylsulfonylcyclopropyl)-6-morpholin-4-ylpyrimidin-2-yl]phenyl]urea,
1-[4-[4-(1-cyclopropylsulfonylcyclopropyl)-6-morpholin-4-ylpyrimidin-2-yl]phenyl]-3-(2-hydroxyethyl)urea,
1-[4-[4-(1-cyclopropylsulfonylcyclopropyl)-6-morpholin-4-ylpyrimidin-2-yl]phenyl]-3-(1-hydroxy-2-methylpropan-2-yl)urea,
1-[4-[4-(1-cyclopropylsulfonylcyclopropyl)-6-morpholin-4-ylpyrimidin-2-yl]phenyl]-3-(2-dimethylaminoethyl)urea,
1-[4-[4-(1-cyclopropylsulfonylcyclopropyl)-6-morpholin-4-ylpyrimidin-2-yl]phenyl]-3-propylurea,
1-[4-[4-(1-cyclopropylsulfonylcyclopropyl)-6-morpholin-4-ylpyrimidin-2-yl]phenyl]-3-(3-hydroxypropyl)urea,
1-[4-[4-(1-cyclopropylsulfonylcyclopropyl)-6-morpholin-4-ylpyrimidin-2-yl]phenyl]-3-(1-methylpyrazol-4-yl)urea,
3-cyclopropyl-1-[4-[4-(1-cyclopropylsulfonylcyclopentyl)-6-morpholin-4-ylpyrimidin-2-yl]phenyl]urea,
1-[4-[4-(1-cyclopropylsulfonylcyclopentyl)-6-morpholin-4-ylpyrimidin-2-yl]phenyl]-3-methylurea,
1-[4-[4-(1-cyclopropylsulfonylcyclopentyl)-6-morpholin-4-ylpyrimidin-2-yl]phenyl]-3-(2-hydroxyethyl)urea,
1-[4-[4-(1-cyclopropylsulfonylcyclopentyl)-6-morpholin-4-ylpyrimidin-2-yl]phenyl]-3-(1-hydroxy-2-methylpropan-2-yl)urea,
1-[4-[4-(1-cyclopropylsulfonylcyclopentyl)-6-morpholin-4-ylpyrimidin-2-yl]phenyl]-3-(2-dimethylaminoethyl)urea,
1-[4-[4-(1-cyclopropylsulfonylcyclopentyl)-6-morpholin-4-ylpyrimidin-2-yl]phenyl]-3-(3-hydroxypropyl)urea,
3-cyclopropyl-1-[4-[4-(1-cyclopropylsulfonylcyclobutyl)-6-morpholin-4-ylpyrimidin-2-yl]phenyl]urea,
1-[4-[4-(1-cyclopropylsulfonylcyclobutyl)-6-morpholin-4-ylpyrimidin-2-yl]phenyl]-3-methylurea,
1-[4-[4-(1-cyclopropylsulfonylcyclobutyl)-6-morpholin-4-ylpyrimidin-2-yl]phenyl]-3-(2-hydroxyethyl)urea,
1-[4-[4-(1-cyclopropylsulfonylcyclobutyl)-6-morpholin-4-ylpyrimidin-2-yl]phenyl]-3-(1-hydroxy-2-methylpropan-2-yl)urea,
1-[4-[4-(1-cyclopropylsulfonylcyclobutyl)-6-morpholin-4-ylpyrimidin-2-yl]phenyl]-3-(2-dimethylaminoethyl)urea,
1-[4-[4-(1-cyclopropylsulfonylcyclobutyl)-6-morpholin-4-ylpyrimidin-2-yl]phenyl]-3-(3-hydroxypropyl)urea,
3-cyclopropyl-1-[5-[4-(1-cyclopropylsulfonylcyclopropyl)-6-[(3S)-3-methylmorpholin-4-yl]pyrimidin-2-yl]pyridin-2-yl]urea,
1-[5-[4-(1-cyclopropylsulfonylcyclopropyl)-6-[(3S)-3-methylmorpholin-4-yl]pyrimidin-2-yl]pyridin-2-yl]-3-methylurea,
1-[5-[4-(1-cyclopropylsulfonylcyclopropyl)-6-[(3S)-3-methylmorpholin-4-yl]pyrimidin-2-yl]pyridin-2-yl]-3-(2-dimethylaminoethyl)urea,
1-[5-[4-(1-cyclopropylsulfonylcyclopropyl)-6-[(3S)-3-methylmorpholin-4-yl]pyrimidin-2-yl]pyridin-2-yl]-3-(2-hydroxyethyl)urea,
1-[5-[4-(1-cyclopropylsulfonylcyclopropyl)-6-[(3S)-3-methylmorpholin-4-yl]pyrimidin-2-yl]pyridin-2-yl]-3-(1-methylpyrazol-4-yl)urea,
3-cyclopropyl-1-[5-[4-[1-(4-fluorophenyl)sulfonylcyclopropyl]-6-[(3S)-3-methylmorpholin-4-yl]pyrimidin-2-yl]pyrimidin-2-yl]urea,
1-[5-[4-[1-(4-fluorophenyl)sulfonylcyclopropyl]-6-[(3S)-3-methylmorpholin-4-yl]pyrimidin-2-yl]pyrimidin-2-yl]-3-methylurea,
1-[5-[4-[1-(4-fluorophenyl)sulfonylcyclopropyl]-6-[(3S)-3-methylmorpholin-4-yl]pyrimidin-2-yl]pyrimidin-2-yl]-3-(2-hydroxyethyl)urea,
1-[5-[4-[1-(4-fluorophenyl)sulfonylcyclopropyl]-6-[(3S)-3-methylmorpholin-4-yl]pyrimidin-2-yl]pyrimidin-2-yl]-3-(1-methylpyrazol-4-yl)urea,
3-cyclopropyl-1-[5-[4-[1-(4-fluorophenyl)sulfonylcyclopropyl]-6-[(3S)-3-methylmorpholin-4-yl]pyrimidin-2-yl]pyridin-2-yl]urea,
1-[5-[4-[1-(4-fluorophenyl)sulfonylcyclopropyl]-6-[(3S)-3-methylmorpholin-4-yl]pyrimidin-2-yl]pyridin-2-yl]-3-methylurea,
3-(2-dimethylaminoethyl)-1-[5-[4-[1-(4-fluorophenyl)sulfonylcyclopropyl]-6-[(3S)-3-methylmorpholin-4-yl]pyrimidin-2-yl]pyridin-2-yl]urea,
1-[5-[4-[1-(4-fluorophenyl)sulfonylcyclopropyl]-6-[(3S)-3-methylmorpholin-4-yl]pyrimidin-2-yl]pyridin-2-yl]-3-(2-hydroxyethyl)urea,
1-[5-[4-[1-(4-fluorophenyl)sulfonylcyclopropyl]-6-[(3S)-3-methylmorpholin-4-yl]pyrimidin-2-yl]pyridin-2-yl]-3-(1-methylpyrazol-4-yl)urea,
3-cyclopropyl-1-[5-[4-[(3S)-3-methylmorpholin-4-yl]-6-(1-methylsulfonylcyclopentyl)pyrimidin-2-yl]pyridin-2-yl]urea,
3-methyl-1-[5-[4-[(3S)-3-methylmorpholin-4-yl]-6-(1-methylsulfonylcyclopentyl)pyrimidin-2-yl]pyridin-2-yl]urea,
3-(2-dimethylaminoethyl)-1-[5-[4-[(3S)-3-methylmorpholin-4-yl]-6-(1-methylsulfonylcyclopentyl)pyrimidin-2-yl]pyridin-2-yl]urea,
3-(2-hydroxyethyl)-1-[5-[4-[(3S)-3-methylmorpholin-4-yl]-6-(1-methylsulfonylcyclopentyl)pyrimidin-2-yl]pyridin-2-yl]urea,
1-[5-[4-[(3S)-3-methylmorpholin-4-yl]-6-(1-methylsulfonylcyclopentyl)pyrimidin-2-yl]pyridin-2-yl]-3-(1-methylpyrazol-4-yl)urea,
3-cyclopropyl-1-[5-[4-[(3S)-3-methylmorpholin-4-yl]-6-(1-methylsulfonylcyclopentyl)pyrimidin-2-yl]pyrimidin-2-yl]urea,
3-methyl-1-[5-[4-[(3S)-3-methylmorpholin-4-yl]-6-(1-methylsulfonylcyclopentyl)pyrimidin-2-yl]pyrimidin-2-yl]urea,
3-(2-hydroxyethyl)-1-[5-[4-[(3S)-3-methylmorpholin-4-yl]-6-(1-methylsulfonylcyclopentyl)pyrimidin-2-yl]pyrimidin-2-yl]urea,
1-[5-[4-[(3S)-3-methylmorpholin-4-yl]-6-(1-methylsulfonylcyclopentyl)pyrimidin-2-yl]pyrimidin-2-yl]-3-(1-methylpyrazol-4-yl)urea,
3-cyclopropyl-1-[5-[4-(1-cyclopropylsulfonylcyclopropyl)-6-[(3S)-3-methylmorpholin-4-yl]pyrimidin-2-yl]pyrimidin-2-yl]urea,
1-[5-[4-(1-cyclopropylsulfonylcyclopropyl)-6-[(3S)-3-methylmorpholin-4-yl]pyrimidin-2-yl]pyrimidin-2-yl]-3-methylurea,
1-[5-[4-(1-cyclopropylsulfonylcyclopropyl)-6-[(3S)-3-methylmorpholin-4-yl]pyrimidin-2-yl]pyrimidin-2-yl]-3-(2-hydroxyethyl)urea,
1-[5-[4-(1-cyclopropylsulfonylcyclopropyl)-6-[(3S)-3-methylmorpholin-4-yl]pyrimidin-2-yl]pyrimidin-2-yl]-3-(1-methylpyrazol-4-yl)urea,
3-(2-hydroxyethyl)-1-[5-[4-[1-[4-(2-hydroxyethylamino)phenyl]sulfonylcyclopropyl]-6-[(3S)-3-methylmorpholin-4-yl]pyrimidin-2-yl]pyrimidin-2-yl]urea,
3-cyclopropyl-1-[4-[4-[1-(2-hydroxyethylsulfonyl)cyclopropyl]-6-[(3S)-3-methylmorpholin-4-yl]pyrimidin-2-yl]phenyl]urea,
3-ethyl-1-[4-[4-[1-(2-hydroxyethylsulfonyl)cyclopropyl]-6-[(3S)-3-methylmorpholin-4-yl]pyrimidin-2-yl]phenyl]urea,
1-[4-[4-[1-(2-hydroxyethylsulfonyl)cyclopropyl]-6-[(3S)-3-methylmorpholin-4-yl]pyrimidin-2-yl]phenyl]-3-methylurea,
3-cyclobutyl-1-[4-[4-[1-(2-hydroxyethylsulfonyl)cyclopropyl]-6-[(3S)-3-methylmorpholin-4-yl]pyrimidin-2-yl]phenyl]urea,
3-(2-hydroxyethyl)-1-[4-[4-[1-(2-hydroxyethylsulfonyl)cyclopropyl]-6-[(3S)-3-methylmorpholin-4-yl]pyrimidin-2-yl]phenyl]urea,
1-[4-[4-[1-(2-hydroxyethylsulfonyl)cyclopropyl]-6-[(3S)-3-methylmorpholin-4-yl]pyrimidin-2-yl]phenyl]-3-(1,2,4-thiadiazol-5-yl)urea,
1-[4-[4-[1-(2-hydroxyethylsulfonyl)cyclopropyl]-6-[(3S)-3-methylmorpholin-4-yl]pyrimidin-2-yl]phenyl]-3-(1-methylpyrazol-4-yl)urea,
3-[4-[4-[1-(2-hydroxyethylsulfonyl)cyclopropyl]-6-[(3S)-3-methylmorpholin-4-yl]pyrimidin-2-yl]phenyl]-1-propylurea,
1-[4-[4-[1-(2-hydroxyethylsulfonyl)cyclopropyl]-6-[(3S)-3-methylmorpholin-4-yl]pyrimidin-2-yl]phenyl]-3-(3-hydroxypropyl)urea,
3-(2-cyanoethyl)-1-[4-[4-[1-(2-hydroxyethylsulfonyl)cyclopropyl]-6-[(3S)-3-methylmorpholin-4-yl]pyrimidin-2-yl]phenyl]urea,
1-[4-[4-[1-(2-hydroxyethylsulfonyl)cyclopropyl]-6-[(3S)-3-methylmorpholin-4-yl]pyrimidin-2-yl]phenyl]-3-(1H-imidazol-2-ylmethyl)urea,
1-[4-[4-[1-(2-hydroxyethylsulfonyl)cyclopropyl]-6-[(3S)-3-methylmorpholin-4-yl]pyrimidin-2-yl]phenyl]-3-[1-(hydroxymethyl)cyclopropyl]urea,
1-[4-[4-[1-(5-fluoropyridin-3-yl)sulfonylcyclopropyl]-6-[(3S)-3-methylmorpholin-4-yl]pyrimidin-2-yl]phenyl]-3-(2-hydroxyethyl)urea,
3-ethyl-1-[4-[4-[1-(5-fluoropyridin-3-yl)sulfonylcyclopropyl]-6-[(3S)-3-methylmorpholin-4-yl]pyrimidin-2-yl]phenyl]urea,
1-[4-[4-[1-(5-fluoropyridin-3-yl)sulfonylcyclopropyl]-6-[(3S)-3-methylmorpholin-4-yl]pyrimidin-2-yl]phenyl]-3-methylurea,
3-cyclopropyl-1-[4-[4-[1-(5-fluoropyridin-3-yl)sulfonylcyclopropyl]-6-[(3S)-3-methylmorpholin-4-yl]pyrimidin-2-yl]phenyl]urea,
1-[4-[4-(1-tert-butylsulfonylcyclopropyl)-6-[(3S)-3-methylmorpholin-4-yl]pyrimidin-2-yl]phenyl]-3-cyclopropylurea,
1-[4-[4-(1-tert-butylsulfonylcyclopropyl)-6-[(3S)-3-methylmorpholin-4-yl]pyrimidin-2-yl]phenyl]-3-cyclobutylurea,
1-[4-[4-(1-tert-butylsulfonylcyclopropyl)-6-[(3S)-3-methylmorpholin-4-yl]pyrimidin-2-yl]phenyl]-3-pyridin-2-ylurea,
1-[4-[4-(1-tert-butylsulfonylcyclopropyl)-6-[(3S)-3-methylmorpholin-4-yl]pyrimidin-2-yl]phenyl]-3-(2-methylpropyl)urea,
1-[4-[4-(1-tert-butylsulfonylcyclopropyl)-6-[(3S)-3-methylmorpholin-4-yl]pyrimidin-2-yl]phenyl]-3-propan-2-ylurea,
1-[4-[4-(1-tert-butylsulfonylcyclopropyl)-6-[(3S)-3-methylmorpholin-4-yl]pyrimidin-2-yl]phenyl]-3-ethylurea,
1-[4-[4-(1-tert-butylsulfonylcyclopropyl)-6-[(3S)-3-methylmorpholin-4-yl]pyrimidin-2-yl]phenyl]-3-(2-dimethylaminoethyl)urea,
1-[4-[4-(1-tert-butylsulfonylcyclopropyl)-6-[(3S)-3-methylmorpholin-4-yl]pyrimidin-2-yl]phenyl]-3-(2-hydroxyethyl)urea,
1-[4-[4-(1-tert-butylsulfonylcyclopropyl)-6-[(3S)-3-methylmorpholin-4-yl]pyrimidin-2-yl]phenyl]-3-propylurea,
1-[4-[4-(1-tert-butylsulfonylcyclopropyl)-6-[(3S)-3-methylmorpholin-4-yl]pyrimidin-2-yl]phenyl]-3-methylurea,
1-[4-[4-(1-tert-butylsulfonylcyclopropyl)-6-[(3S)-3-methylmorpholin-4-yl]pyrimidin-2-yl]phenyl]-3-[4-(trifluoromethyl)phenyl]urea,
1-[4-[4-(1-tert-butylsulfonylcyclopropyl)-6-[(3S)-3-methylmorpholin-4-yl]pyrimidin-2-yl]phenyl]-3-(1-hydroxy-2-methylpropan-2-yl)urea,
1-[4-[4-(1-tert-butylsulfonylcyclopropyl)-6-[(3S)-3-methylmorpholin-4-yl]pyrimidin-2-yl]phenyl]-3-(3-hydroxypropyl)urea,
1-[4-[4-(1-tert-butylsulfonylcyclopropyl)-6-[(3S)-3-methylmorpholin-4-yl]pyrimidin-2-yl]phenyl]-3-(1-methylpyrazol-4-yl)urea,
3-cyclopropyl-1-[4-[4-[1-(3,5-difluorophenyl)sulfonylcyclopropyl]-6-[(3S)-3-methylmorpholin-4-yl]pyrimidin-2-yl]phenyl]urea,
3-cyclobutyl-1-[4-[4-[1-(3,5-difluorophenyl)sulfonylcyclopropyl]-6-[(3S)-3-methylmorpholin-4-yl]pyrimidin-2-yl]phenyl]urea,
1-[4-[4-[1-(3,5-difluorophenyl)sulfonylcyclopropyl]-6-[(3S)-3-methylmorpholin-4-yl]pyrimidin-2-yl]phenyl]-3-pyridin-2-ylurea,
1-[4-[4-[1-(3,5-difluorophenyl)sulfonylcyclopropyl]-6-[(3S)-3-methylmorpholin-4-yl]pyrimidin-2-yl]phenyl]-3-(2-methylpropyl)urea,
1-[4-[4-[1-(3,5-difluorophenyl)sulfonylcyclopropyl]-6-[(3S)-3-methylmorpholin-4-yl]pyrimidin-2-yl]phenyl]-3-propan-2-ylurea,
1-[4-[4-[1-(3,5-difluorophenyl)sulfonylcyclopropyl]-6-[(3S)-3-methylmorpholin-4-yl]pyrimidin-2-yl]phenyl]-3-ethylurea,
1-[4-[4-[1-(3,5-difluorophenyl)sulfonylcyclopropyl]-6-[(3S)-3-methylmorpholin-4-yl]pyrimidin-2-yl]phenyl]-3-(2-dimethylaminoethyl)urea,
1-[4-[4-[1-(3,5-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(3,5-difluorophenyl)sulfonylcyclopropyl]-6-[(3S)-3-methylmorpholin-4-yl]pyrimidin-2-yl]phenyl]-3-propylurea,
1-[4-[4-[1-(3,5-difluorophenyl)sulfonylcyclopropyl]-6-[(3S)-3-methylmorpholin-4-yl]pyrimidin-2-yl]phenyl]-3-methylurea,
1-[4-[4-[1-(3,5-difluorophenyl)sulfonylcyclopropyl]-6-[(3S)-3-methylmorpholin-4-yl]pyrimidin-2-yl]phenyl]-3-[4-(trifluoromethyl)phenyl]urea,
1-[4-[4-[1-(3,5-difluorophenyl)sulfonylcyclopropyl]-6-[(3S)-3-methylmorpholin-4-yl]pyrimidin-2-yl]phenyl]-3-(1-hydroxy-2-methylpropan-2-yl)urea,
1-[4-[4-[1-(3,5-difluorophenyl)sulfonylcyclopropyl]-6-[(3S)-3-methylmorpholin-4-yl]pyrimidin-2-yl]phenyl]-3-(3-hydroxypropyl)urea,
1-[4-[4-[1-(3,5-difluorophenyl)sulfonylcyclopropyl]-6-[(3S)-3-methylmorpholin-4-yl]pyrimidin-2-yl]phenyl]-3-(1-methylpyrazol-4-yl)urea,
3-cyclopropyl-1-[4-[4-[(3S)-3-methylmorpholin-4-yl]-6-(4-methylsulfonylpiperidin-4-yl)pyrimidin-2-yl]phenyl]urea,
3-methyl-1-[4-[4-[(3S)-3-methylmorpholin-4-yl]-6-(4-methylsulfonylpiperidin-4-yl)pyrimidin-2-yl]phenyl]urea,
3-(2-dimethylaminoethyl)-1-[4-[4-[(3S)-3-methylmorpholin-4-yl]-6-(4-methylsulfonylpiperidin-4-yl)pyrimidin-2-yl]phenyl]urea,
3-(2-hydroxyethyl)-1-[4-[4-[(3S)-3-methylmorpholin-4-yl]-6-(4-methylsulfonylpiperidin-4-yl)pyrimidin-2-yl]phenyl]urea,
1-[4-[4-[(3S)-3-methylmorpholin-4-yl]-6-(4-methylsulfonylpiperidin-4-yl)pyrimidin-2-yl]phenyl]-3-(1-methylpyrazol-4-yl)urea,
3-cyclopropyl-1-[4-[4-(4-cyclopropylsulfonylpiperidin-4-yl)-6-[(3S)-3-methylmorpholin-4-yl]pyrimidin-2-yl]phenyl]urea,
1-[4-[4-(4-cyclopropylsulfonylpiperidin-4-yl)-6-[(3S)-3-methylmorpholin-4-yl]pyrimidin-2-yl]phenyl]-3-methylurea,
1-[4-[4-(4-cyclopropylsulfonylpiperidin-4-yl)-6-[(3S)-3-methylmorpholin-4-yl]pyrimidin-2-yl]phenyl]-3-(2-hydroxyethyl)urea,
1-[4-[4-(4-cyclopropylsulfonylpiperidin-4-yl)-6-[(3S)-3-methylmorpholin-4-yl]pyrimidin-2-yl]phenyl]-3-(1-methylpyrazol-4-yl)urea,
1-[4-[4-[1-Benzyl-4-(3,5-difluorophenyl)sulfonylpiperidin-4-yl]-6-[(3S)-3-methylmorpholin-4-yl]pyrimidin-2-yl]phenyl]-3-cyclopropylurea,
3-cyclopropyl-1-[4-[4-[(3S)-3-methylmorpholin-4-yl]-6-(4-methylsulfonyloxan-4-yl)pyrimidin-2-yl]phenyl]urea,
3-methyl-1-[4-[4-[(3S)-3-methylmorpholin-4-yl]-6-(4-methylsulfonyloxan-4-yl)pyrimidin-2-yl]phenyl]urea,
3-(2-dimethylaminoethyl)-1-[4-[4-[(3S)-3-methylmorpholin-4-yl]-6-(4-methylsulfonyloxan-4-yl)pyrimidin-2-yl]phenyl]urea,
3-(2-hydroxyethyl)-1-[4-[4-[(3S)-3-methylmorpholin-4-yl]-6-(4-methylsulfonyloxan-4-yl)pyrimidin-2-yl]phenyl]urea,
1-[4-[4-[(3S)-3-methylmorpholin-4-yl]-6-(4-methylsulfonyloxan-4-yl)pyrimidin-2-yl]phenyl]-3-(1-methylpyrazol-4-yl)urea,
3-cyclopropyl-1-[4-[4-(4-cyclopropylsulfonyloxan-4-yl)-6-[(3S)-3-methylmorpholin-4-yl]pyrimidin-2-yl]phenyl]urea,
1-[4-[4-(4-cyclopropylsulfonyloxan-4-yl)-6-[(3S)-3-methylmorpholin-4-yl]pyrimidin-2-yl]phenyl]-3-methylurea,
1-[4-[4-(4-cyclopropylsulfonyloxan-4-yl)-6-[(3S)-3-methylmorpholin-4-yl]pyrimidin-2-yl]phenyl]-3-(2-dimethylaminoethyl)urea,
1-[4-[4-(4-cyclopropylsulfonyloxan-4-yl)-6-[(3S)-3-methylmorpholin-4-yl]pyrimidin-2-yl]phenyl]-3-(2-hydroxyethyl)urea,
1-[4-[4-(4-cyclopropylsulfonyloxan-4-yl)-6-[(3S)-3-methylmorpholin-4-yl]pyrimidin-2-yl]phenyl]-3-(1-methylpyrazol-4-yl)urea,
1-[4-[4-[4-(4-chlorophenyl)sulfonyloxan-4-yl]-6-[(3S)-3-methylmorpholin-4-yl]pyrimidin-2-yl]phenyl]-3-methylurea,
1-[4-[4-[4-(4-chlorophenyl)sulfonyloxan-4-yl]-6-[(3S)-3-methylmorpholin-4yl]pyrimidin-2-yl]phenyl]-3-(2-dimethylaminoethyl)urea,
1-[4-[4-[4-(4-chlorophenyl)sulfonyloxan-4-yl]-6-[(3S)-3-methylmorpholin-4-yl]pyrimidin-2-yl]phenyl]-3-(2-hydroxyethyl)urea,
1-[4-[4-[4-(4-chlorophenyl)sulfonyloxan-4-yl]-6-[(3S)-3-methylmorpholin-4-yl]pyrimidin-2-yl]phenyl]-3-(1-methylpyrazol-4-yl)urea,
1-[4-[4-[4-(4-chlorophenyl)sulfonyloxan-4-yl]-6-[(3S)-3-methylmorpholin-4-yl]pyrimidin-2-yl]phenyl]-3-cyclopropylurea,
3-cyclopropyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-cyclobutyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
1-[4-[4-[(3S)-3-methylmorpholin-4-yl]-6-[1-[(4-methyl-1,3-thiazo-2-yl)sulfonyl]cyclopropyl]pyrimidin-2-yl]phenyl]-3-pyridin-2-ylurea,
1-[4-[4-[(3S)-3-methylmorpholin-4-yl]-6-[1-[(4-methyl-1,3-thiazo-2-yl)sulfonyl]cyclopropyl]pyrimidin-2-yl]phenyl]-3-(2-methylpropyl)urea,
1-[4-[4-[(3S)-3-methylmorpholin-4-yl]-6-[1-[(4-methyl-1,3-thiazol-2-yl)sulfonyl]cyclopropyl]pyrimidin-2-yl]phenyl]-3-propan-2-ylurea,
1-ethyl-3-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-dimethylaminoethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-hydroxyethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-[4-[4-[(3S)-3-methylmorpholin-4-yl]-6-[1-[(4-methyl-1,3-thiazol-2-yl)sulfonyl]cyclopropyl]pyrimidin-2-yl]phenyl]-1-propylurea,
3-methyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
1-[4-[4-[(3S)-3-methylmorpholin-4-yl]-6-[1-[(4-methyl-1,3-thiazo-2-yl)sulfonyl]cyclopropyl]pyrimidin-2-yl]phenyl]-3-[4-(trifluoromethyl)phenyl]urea,
3-(1-hydroxy-2-methylpropan-2-yl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(3-hydroxypropyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
1-[4-[4-[(3S)-3-methylmorpholin-4-yl]-6-[1-[(4-methyl-1,3-thiazo-2-yl)sulfonyl]cyclopropyl]pyrimidin-2-yl]phenyl]-3-(1-methylpyrazol-4-yl)urea,
1-[4-[4-(1-cyclohexylsulfonylcyclopropyl)-6-[(3S)-3-methylmorpholin-4-yl]pyrimidin-2-yl]phenyl]-3-cyclopropylurea,
3-cyclobutyl-1-[4-[4-(1-cyclohexylsulfonylcyclopropyl)-6-[(3S)-3-methylmorpholin-4-yl]pyrimidin-2-yl]phenyl]urea,
3-[4-[4-(1-cyclohexylsulfonylcyclopropyl)-6-[(3S)-3-methylmorpholin-4-yl]pyrimidin-2-yl]phenyl]-1-ethylurea,
1-[4-[4-(1-cyclohexylsulfonylcyclopropyl)-6-[(3S)-3-methylmorpholin-4-yl]pyrimidin-2-yl]phenyl]-3-(2-dimethylaminoethyl)urea,
1-[4-[4-(1-cyclohexylsulfonylcyclopropyl)-6-[(3S)-3-methylmorpholin-4-yl]pyrimidin-2-yl]phenyl]-3-(2-hydroxyethyl)urea,
1-[4-[4-(1-cyclohexylsulfonylcyclopropyl)-6-[(3S)-3-methylmorpholin-4-yl]pyrimidin-2-yl]phenyl]-3-methylurea,
1-[4-[4-(1-cyclohexylsulfonylcyclopropyl)-6-[(3S)-3-methylmorpholin-4-yl]pyrimidin-2-yl]phenyl]-3-(1-hydroxy-2-methylpropan-2-yl)urea,
1-[4-[4-(1-cyclohexylsulfonylcyclopropyl)-6-[(3S)-3-methylmorpholin-4-yl]pyrimidin-2-yl]phenyl]-3-(3-hydroxypropyl)urea,
1-[4-[4-(1-cyclohexylsulfonylcyclopropyl)-6-[(3S)-3-methylmorpholin-4-yl]pyrimidin-2-yl]phenyl]-3-(1-methylpyrazol-4-yl)urea,
1-[4-[4-[1-(4-chlorophenyl)sulfonylcyclopropyl]-6-[(3S)-3-methylmorpholin-4-yl]pyrimidin-2-yl]phenyl]-3-cyclopropylurea,
1-[4-[4-[1-(4-chlorophenyl)sulfonylcyclopropyl]-6-[(3S)-3-methylmorpholin-4-yl]pyrimidin-2-yl]phenyl]-3-cyclobutylurea,
1-[4-[4-[1-(4-chlorophenyl)sulfonylcyclopropyl]-6-[(3S)-3-methylmorpholin-4-yl]pyrimidin-2-yl]phenyl]-3-pyridin-2-ylurea,
1-[4-[4-[1-(4-chlorophenyl)sulfonylcyclopropyl]-6-[(3S)-3-methylmorpholin-4-yl]pyrimidin-2-yl]phenyl]-3-(2-methylpropyl)urea,
1-[4-[4-[1-(4-chlorophenyl)sulfonylcyclopropyl]-6-[(3S)-3-methylmorpholin-4-yl]pyrimidin-2-yl]phenyl]-3-propan-2-ylurea,
1-[4-[4-[1-(4-chlorophenyl)sulfonylcyclopropyl]-6-[(3S)-3-methylmorpholin-4-yl]pyrimidin-2-yl]phenyl]-3-methylurea,
3-[4-[4-[1-(4-chlorophenyl)sulfonylcyclopropyl]-6-[(3S)-3-methylmorpholin-4-yl]pyrimidin-2-yl]phenyl]-1-ethylurea,
1-[4-[4-[1-(4-chlorophenyl)sulfonylcyclopropyl]-6-[(3S)-3-methylmorpholin-4-yl]pyrimidin-2-yl]phenyl]-3-(2-dimethylaminoethyl)urea,
1-[4-[4-[1-(4-chlorophenyl)sulfonylcyclopropyl]-6-[(3S)-3-methylmorpholin-4-yl]pyrimidin-2-yl]phenyl]-3-(2-hydroxyethyl)urea,
3-[4-[4-[1-(4-chlorophenyl)sulfonylcyclopropyl]-6-[(3S)-3-methylmorpholin-4-yl]pyrimidin-2-yl]phenyl]-1-propylurea,
1-[4-[4-[1-(4-chlorophenyl)sulfonylcyclopropyl]-6-[(3S)-3-methylmorpholin-4-yl]pyrimidin-2-yl]phenyl]-3-[4-(trifluoromethyl)phenyl]urea,
1-[4-[4-[1-(4-chlorophenyl)sulfonylcyclopropyl]-6-[(3S)-3-methylmorpholin-4-yl]pyrimidin-2-yl]phenyl]-3-(1-hydroxy-2-methylpropan-2-yl)urea,
1-[4-[4-[1-(4-chlorophenyl)sulfonylcyclopropyl]-6-[(3S)-3-methylmorpholin-4-yl]pyrimidin-2-yl]phenyl]-3-(3-hydroxypropyl)urea,
1-[4-[4-[1-(4-chlorophenyl)sulfonylcyclopropyl]-6-[(3S)-3-methylmorpholin-4-yl]pyrimidin-2-yl]phenyl]-3-(1-methylpyrazol-4-yl)urea,
3-cyclopropyl-1-[4-[4-[(3S)-3-methylmorpholin-4-yl]-6-(1-pyridin-2-ylsulfonylcyclopropyl)pyrimidin-2-yl]phenyl]urea,
3-cyclobutyl-1-[4-[4-[(3S)-3-methylmorpholin-4-yl]-6-(1-pyridin-2-ylsulfonylcyclopropyl)pyrimidin-2-yl]phenyl]urea,
1-[4-[4-[(3S)-3-methylmorpholin-4-yl]-6-(1-pyridin-2-ylsulfonylcyclopropyl)pyrimidin-2-yl]phenyl]-3-pyridin-2-ylurea,
1-[4-[4-[(3S)-3-methylmorpholin-4-yl]-6-(1-pyridin-2-ylsulfonylcyclopropyl)pyrimidin-2-yl]phenyl]-3-(2-methylpropyl)urea,
1-[4-[4-[(3S)-3-methylmorpholin-4-yl]-6-(1-pyridin-2-ylsulfonylcyclopropyl)pyrimidin-2-yl]phenyl]-3-propan-2-ylurea,
3-methyl-1-[4-[4-[(3S)-3-methylmorpholin-4-yl]-6-(1-pyridin-2-ylsulfonylcyclopropyl)pyrimidin-2-yl]phenyl]urea,
3-(2-dimethylaminoethyl)-1-[4-[4-[(3S)-3-methylmorpholin-4-yl]-6-(1-pyridin-2-ylsulfonylcyclopropyl)pyrimidin-2-yl]phenyl]urea,
3-(2-hydroxyethyl)-1-[4-[4-[(3S)-3-methylmorpholin-4-yl]-6-(1-pyridin-2-ylsulfonylcyclopropyl)pyrimidin-2-yl]phenyl]urea,
3-[4-[4-[(3S)-3-methylmorpholin-4-yl]-6-(1-pyridin-2-ylsulfonylcyclopropyl)pyrimidin-2-yl]phenyl]-1-propylurea,
1-[4-[4-[(3S)-3-methylmorpholin-4-yl]-6-(1-pyridin-2-ylsulfonylcyclopropyl)pyrimidin-2-yl]phenyl]-3-[4-(trifluoromethyl)phenyl]urea,
3-(1-hydroxy-2-methylpropan-2-yl)-1-[4-[4-[(3S)-3-methylmorpholin-4-yl]-6-(1-pyridin-2-ylsulfonylcyclopropyl)pyrimidin-2-yl]phenyl]urea,
3-(3-hydroxypropyl)-1-[4-[4-[(3S)-3-methylmorpholin-4-yl]-6-(1-pyridin-2-ylsulfonylcyclopropyl)pyrimidin-2-yl]phenyl]urea,
1-[4-[4-[(3S)-3-methylmorpholin-4-yl]-6-(1-pyridin-2-ylsulfonylcyclopropyl)pyrimidin-2-yl]phenyl]-3-(1-methylpyrazol-4-yl)urea
[4-[4-[(3S)-3-methylmorpholin-4-yl]-6-(1-methylsulfonylcyclopropyl)pyrimidin-2-yl]phenyl]urea,
3-cyclopropyl-1-[4-[4-[4-(3,5-difluorophenyl)sulfonylpiperidin-4-yl]-6-[(3S)-3-methylmorpholin-4-yl]pyrimidin-2-yl]phenyl]urea,
1-[4-[4-[1-(3-hydroxypropylsulfonyl)cyclopropyl]-6-[(3S)-3-methylmorpholin-4-yl]pyrimidin-2-yl]phenyl]-3-prop an-2-ylurea,
3-cyclopropyl-1-[4-[4-[1-(3-hydroxypropylsulfonyl)cyclopropyl]-6-[(3S)-3-methylmorpholin-4-yl]pyrimidin-2-yl]phenyl]urea,
3-cyclobutyl-1-[4-[4-[1-(3-hydroxypropylsulfonyl)cyclopropyl]-6-[(3S)-3-methylmorpholin-4-yl]pyrimidin-2-yl]phenyl]urea,
1-[4-[4-[1-(3-hydroxypropylsulfonyl)cyclopropyl]-6-[(3S)-3-methylmorpholin-4-yl]pyrimidin-2-yl]phenyl]-3-pyridin-2-ylurea,
1-[4-[4-[1-(3-hydroxypropylsulfonyl)cyclopropyl]-6-[(3S)-3-methylmorpholin-4-yl]pyrimidin-2-yl]phenyl]-3-(2-methylpropyl)urea,
1-ethyl-3-[4-[4-[1-(3-hydroxypropylsulfonyl)cyclopropyl]-6-[(3S)-3-methylmorpholin-4-yl]pyrimidin-2-yl]phenyl]urea,
3-(2-hydroxyethyl)-1-[4-[4-[1-(3-hydroxypropylsulfonyl)cyclopropyl]-6-[(3S)-3-methylmorpholin-4-yl]pyrimidin-2-yl]phenyl]urea,
1-[4-[4-[1-(3-hydroxypropylsulfonyl)cyclopropyl]-6-[(3S)-3-methylmorpholin-4-yl]pyrimidin-2-yl]phenyl]-3-methylurea,
1-[4-[4-[1-(3-hydroxypropylsulfonyl)cyclopropyl]-6-[(3S)-3-methylmorpholin-4-yl]pyrimidin-2-yl]phenyl]-3-[4-(trifluoromethyl)phenyl]urea,
3-(3-hydroxypropyl)-1-[4-[4-[1-(3-hydroxypropylsulfonyl)cyclopropyl]-6-[(3S)-3-methylmorpholin-4-yl]pyrimidin-2-yl]phenyl]urea,
1-[4-[4-[1-(3-hydroxypropylsulfonyl)cyclopropyl]-6-[(3S)-3-methylmorpholin-4-yl]pyrimidin-2-yl]phenyl]-3-(1-methylpyrazol-4-yl)urea,
3-[4-[4-[1-(3-hydroxypropylsulfonyl)cyclopropyl]-6-[(3S)-3-methylmorpholin-4-yl]pyrimidin-2-yl]phenyl]-1-propylurea,
3-(2-dimethylaminoethyl)-1-[4-[4-[1-(3-hydroxypropylsulfonyl)cyclopropyl]-6-[(3S)-3-methylmorpholin-4-yl]pyrimidin-2-yl]phenyl]urea
3-(1-hydroxy-2-methylpropan-2-yl)-1-[4-[4-[1-(3-hydroxypropylsulfonyl)cyclopropyl]-6-[(3S)-3-methylmorpholin-4-yl]pyrimidin-2-yl]phenyl]urea
N,N-dimethyl-1-[2-[4-(methylcarbamoylamino)phenyl]-6-morpholin-4-ylpyrimidin-4-yl]cyclopropane-1-carboxamide
3-methyl-1-[4-[4-[1-[(3S)-3-methylmorpholine-4-carbonyl]cyclopropyl]-6-morpholin-4-ylpyrimidin-2-yl]phenyl]urea
N-cyclopropyl-1-[2-[4-(methylcarbamoylamino)phenyl]-6-morpholin-4-ylpyrimidin-4-yl]cyclopropane-1-carboxamide
N-cyclopropyl-N-methyl-1-[2-[4-(methylcarbamoylamino)phenyl]-6-morpholin-4-ylpyrimidin-4-yl]cyclopropane-1-carboxamide
3-methyl-1-[4-[4-[1-(4-methylpiperazine-1-carbonyl)cyclopropyl]-6-morpholin-4-ylpyrimidin-2-yl]phenyl]urea
1-[2-[4-(cyclopropylcarbamoylamino)phenyl]-6-morpholin-4-ylpyrimidin-4-yl]-N,N-dimethylcyclopropane-1-carboxamide
3-cyclopropyl-1-[4-[4-[1-[(3S)-3-methylmorpholine-4-carbonyl]cyclopropyl]-6-morpholin-4-ylpyrimidin-2-yl]phenyl]urea
N-cyclopropyl-1-[2-[4-(cyclopropylcarbamoylamino)phenyl]-6-morpholin-4-ylpyrimidin-4-yl]cyclopropane-1-carboxamide
N-cyclopropyl-1-[2-[4-(cyclopropylcarbamoylamino)phenyl]-6-morpholin-4-ylpyrimidin-4-yl]-N-methylcyclopropane-1-carboxamide
3-cyclopropyl-1-[4-[4-[1-(4-methylpiperazine-1-carbonyl)cyclopropyl]-6-morpholin-4-ylpyrimidin-2-yl]phenyl]urea
1-[2-[4-(2-hydroxyethylcarbamoylamino)phenyl]-6-morpholin-4-ylpyrimidin-4-yl]-N,N-dimethylcyclopropane-1-carboxamide
3-(2-hydroxyethyl)-1-[4-[4-[1-[(3S)-3-methylmorpholine-4-carbonyl]cyclopropyl]-6-morpholin-4-ylpyrimidin-2-yl]phenyl]urea
N-cyclopropyl-1-[2-[4-(2-hydroxyethylcarbamoylamino)phenyl] -6-morpholin-4-ylpyrimidin-4-yl]cyclopropane-1-carboxamide
N-cyclopropyl-1-[2-[4-(2-hydroxyethylcarbamoylamino)phenyl]-6-morpholin-4-ylpyrimidin-4-yl]-N-methylcyclopropane-1-carboxamide,
3-(2-hydroxyethyl)-1-[4-[4-[1-(4-methylpiperazine-1-carbonyl)cyclopropyl]-6-morpholin-4-ylpyrimidin-2-yl]phenyl]urea,
3-methyl-1-[4-[4-[(3S)-3-methylmorpholin-4-yl]-6-(1-methylsulfonylcyclopropyl)pyrimidin-2-yl]phenyl]thiourea,
3-ethyl-1-[4-[4-[(3S)-3-methylmorpholin-4-yl]-6-(1-methylsulfonylcyclopropyl)pyrimidin-2-yl]phenyl]thiourea,
3-(2-hydroxyethyl)-1-[4-[4-[(3S)-3-methylmorpholin-4-yl]-6-(1-methylsulfonylcyclopropyl)pyrimidin-2-yl]phenyl]thiourea,
3-(2-dimethylaminoethyl)-1-[4-[4-[(3S)-3-methylmorpholin-4-yl]-6-(1-methylsulfonylcyclopropyl)pyrimidin-2-yl]phenyl]thiourea,
3-cyclopropyl-1-[4-[4-[(3S)-3-methylmorpholin-4-yl]-6-(1-methylsulfonylcyclopropyl)pyrimidin-2-yl]phenyl]thiourea,
3-[1-(hydroxymethyl)cyclopropyl]-1-[4-[4-[1-(3-hydroxypropylsulfonyl)cyclopropyl]-6-[(3S)-3-methylmorpholin-4-yl]pyrimidin-2-yl]phenyl]urea,
1-[4-[4-[1-(3-hydroxypropylsulfonyl)cyclopropyl]-6-[(3S)-3-methylmorpholin-4-yl]pyrimidin-2-yl]phenyl]-3-(1,2,4-thiadiazol-5-yl)urea,
1-[4-[4-[1-(3-hydroxypropylsulfonyl)cyclopropyl]-6-[(3S)-3-methylmorpholin-4-yl]pyrimidin-2-yl]phenyl]-3-(1H-imidazol-2-ylmethyl)urea,
1-[4-[4-(1-cyclopropylsulfonylcyclobutyl)-6-[(3S)-3-methylmorpholin-4-yl]pyrimidin-2-yl]phenyl]-3-methylurea,
3-cyclopropyl-1-[4-[4-(1-cyclopropylsulfonylcyclobutyl)-6-[(3S)-3-methylmorpholin-4-yl]pyrimidin-2-yl]phenyl]urea,
1-[4-[4-(1-cyclopropylsulfonylcyclobutyl)-6-[(3S)-3-methylmorpholin-4-yl]pyrimidin-2-yl]phenyl]-3-(2-hydroxyethyl)urea,
3-(2-cyanoethyl)-1-[4-[4-(1-cyclopropylsulfonylcyclobutyl)-6-[(3S)-3-methylmorpholin-4-yl]pyrimidin-2-yl]phenyl]urea,
1-[4-[4-(1-cyclopropylsulfonylcyclobutyl)-6-[(3S)-3-methylmorpholin-4-yl]pyrimidin-2-yl]phenyl]-3-(1-methylpyrazol-4-yl)urea,
1-[4-[4-(1-cyclopropylsulfonylcyclobutyl)-6-[(3S)-3-methylmorpholin-4-yl]pyrimidin-2-yl]phenyl]-3-(1H-imidazol-2-ylmethyl)urea,
3-cyclopropyl-1-[4-[4-(1-cyclopropylsulfonylcyclopentyl)-6-[(3S)-3-methylmorpholin-4-yl]pyrimidin-2-yl]phenyl]urea,
1-[4-[4-(1-cyclopropylsulfonylcyclopentyl)-6-[(3S)-3-methylmorpholin-4-yl]pyrimidin-2-yl]phenyl]-3-(2-hydroxyethyl)urea,
3-(2-cyanoethyl)-1-[4-[4-(1-cyclopropylsulfonylcyclopentyl)-6-[(3S)-3-methylmorpholin-4-yl]pyrimidin-2-yl]phenyl]urea,
1-[4-[4-(1-cyclopropylsulfonylcyclopentyl)-6-[(3S)-3-methylmorpholin-4-yl]pyrimidin-2-yl]phenyl]-3-(3-hydroxypropyl)urea,
3-(1H-imidazol-2-ylmethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-cyanoethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
1-[4-[4-[(3S)-3-methylmorpholin-4-yl]-6-[1-[(4-methyl-1,3-thiazo-2-yl)sulfonyl]cyclopropyl]pyrimidin-2-yl]phenyl]-3-(1,2,4-thiadiazol-5-yl)urea,
3-[1-(hydroxymethyl)cyclopropyl]-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-cyanoethyl)-1-[4-[4-(1-methylsulfonylcyclopropyl)-6-morpholin-4-ylpyrimidin-2-yl]phenyl]urea,
1-[4-[4-(1-methylsulfonylcyclopropyl)-6-morpholin-4-ylpyrimidin-2-yl]phenyl]-3-(1,2,4-thiadiazol-5-yl)urea,
3-(1H-imidazol-2-ylmethyl)-1-[4-[4-(1-methylsulfonylcyclopropyl)-6-morpholin-4-ylpyrimidin-2-yl]phenyl]urea,
3-(2-cyanoethyl)-1-[4-[4-(1-ethylsulfonylcyclopropyl)-6-[(3S)-3-methylmorpholin-4-yl]pyrimidin-2-yl]phenyl]urea,
1-[4-[4-(1-ethylsulfonylcyclopropyl)-6-[(3S)-3-methylmorpholin-4-yl]pyrimidin-2-yl]phenyl]-3-[1-(hydroxymethyl)cyclopropyl]urea,
1-[4-[4-(1-ethylsulfonylcyclopropyl)-6-[(3S)-3-methylmorpholin-4-yl]pyrimidin-2-yl]phenyl]-3-(1H-imidazol-2-ylmethyl)urea,
1-[4-[4-[1-(4-fluorophenyl)sulfonylcyclopropyl]-6-[(3S)-3-methylmorpholin-4-yl]pyrimidin-2-yl]phenyl]-3-[1-(hydroxymethyl)cyclopropyl]urea,
3-(2-cyanoethyl)-1-[4-[4-[1-(4-fluorophenyl)sulfonylcyclopropyl]-6-[(3S)-3-methylmorpholin-4-yl]pyrimidin-2-yl]phenyl]urea,
1-[4-[4-[1-(4-fluorophenyl)sulfonylcyclopropyl]-6-[(3S)-3-methylmorpholin-4-yl]pyrimidin-2-yl]phenyl]-3-(1,2,4-thiadiazol-5-yl)urea,
1-[4-[4-[1-(4-fluorophenyl)sulfonylcyclopropyl]-6-[(3S)-3-methylmorpholin-4-yl]pyrimidin-2-yl]phenyl]-3-(1H-imidazol-2-ylmethyl)urea,
3-cyclopropyl-1-[4-[4-[(3S)-3-methylmorpholin-4-yl]-6-(1-pyridin-2-ylsulfonylcyclopentyl)pyrimidin-2-yl]phenyl]urea,
3-(2-hydroxyethyl)-1-[4-[4-[(3S)-3-methylmorpholin-4-yl]-6-(1-pyridin-2-ylsulfonylcyclopentyl)pyrimidin-2-yl]phenyl]urea,
3-(2-cyanoethyl)-1-[4-[4-[(3S)-3-methylmorpholin-4-yl]-6-(1-pyridin-2-ylsulfonylcyclopentyl)pyrimidin-2-yl]phenyl]urea,
3-(3-hydroxypropyl)-1-[4-[4-[(3S)-3-methylmorpholin-4-yl]-6-(1-pyridin-2-ylsulfonylcyclopentyl)pyrimidin-2-yl]phenyl]urea,
3-cyclopropyl-1-[4-[4-[(3S)-3-methylmorpholin-4-yl]-6-(1-pyridin-2-ylsulfonylcyclobutyl)pyrimidin-2-yl]phenyl]urea,
3-methyl-1-[4-[4-[(3S)-3-methylmorpholin-4-yl]-6-(1-pyridin-2-ylsulfonylcyclobutyl)pyrimidin-2-yl]phenyl]urea,
3-(2-hydroxyethyl)-1-[4-[4-[(3S)-3-methylmorpholin-4-yl]-6-(1-pyridin-2-ylsulfonylcyclobutyl)pyrimidin-2-yl]phenyl]urea,
3-(2-cyanoethyl)-1-[4-[4-[(3S)-3-methylmorpholin-4-yl]-6-(1-pyridin-2-ylsulfonylcyclobutyl)pyrimidin-2-yl]phenyl]urea,
1-[4-[4-[(3S)-3-methylmorpholin-4-yl]-6-(1-pyridin-2-ylsulfonylcyclobutyl)pyrimidin-2-yl]phenyl]-3-(1-methylpyrazol-4-yl)urea,
3-(1H-imidazol-2-ylmethyl)-1-[4-[4-[(3S)-3-methylmorpholin-4-yl]-6-(1-pyridin-2-ylsulfonylcyclobutyl)pyrimidin-2-yl]phenyl]urea,
3-cyclopropyl-1-[4-[4-[1-(4-fluorophenyl)sulfonylcyclopropyl]-6-morpholin-4-ylpyrimidin-2-yl]phenyl]urea,
3-(2-cyanoethyl)-1-[4-[4-[1-(4-fluorophenyl)sulfonylcyclopropyl]-6-morpholin-4-ylpyrimidin-2-yl]phenyl]urea,
1-[4-[4-[1-(4-fluorophenyl)sulfonylcyclopropyl]-6-morpholin-4-ylpyrimidin-2-yl]phenyl]-3-(1,2,4-thiadiazol-5-yl)urea,
1-ethyl-3-[4-[4-[1-(4-fluorophenyl)sulfonylcyclopropyl]-6-morpholin-4-ylpyrimidin-2-yl]phenyl]urea,
3-[4-[4-[1-(4-fluorophenyl)sulfonylcyclopropyl]-6-morpholin-4-ylpyrimidin-2-yl]phenyl]-1-methylurea,
3-[4-[4-[1-(4-fluorophenyl)sulfonylcyclopropyl]-6-morpholin-4-ylpyrimidin-2-yl]phenyl]-1-(1-methylpyrazol-4-yl)urea,
3-(2-hydroxyethyl)-1-[4-[4-morpholin-4-yl-6-(1-pyridin-4-ylsulfonylcyclopropyl)pyrimidin-2-yl]phenyl]urea,
1-(1-methylpyrazol-4-yl)-3-[4-[4-morpholin-4-yl-6-(1-pyridin-4-ylsulfonylcyclopropyl)pyrimidin-2-yl]phenyl]urea,
1-methyl-3-[4-[4-morpholin-4-yl-6-(1-pyridin-4-ylsulfonylcyclopropyl)pyrimidin-2-yl]phenyl]urea,
1-ethyl-3-[4-[4-morpholin-4-yl-6-(1-pyridin-4-ylsulfonylcyclopropyl)pyrimidin-2-yl]phenyl]urea,
3-cyclopropyl-1-[4-[4-morpholin-4-yl-6-(1-pyridin-4-ylsulfonylcyclopropyl)pyrimidin-2-yl]phenyl]urea,
3-(2-cyanoethyl)-1-[4-[4-morpholin-4-yl-6-(1-pyridin-4-ylsulfonylcyclopropyl)pyrimidin-2-yl]phenyl]urea,
3-(3-hydroxypropyl)-1-[4-[4-morpholin-4-yl-6-(1-pyridin-4-ylsulfonylcyclopropyl)pyrimidin-2-yl]phenyl]urea,
1-[4-[4-[(3S)-3-methylmorpholin-4-yl]-6-(1-methylsulfonylcyclopropyl)pyrimidin-2-yl]phenyl]-3-(1,3-thiazol-2-yl)urea,
3-[4-[4-[(3S)-3-methylmorpholin-4-yl]-6-(1-methylsulfonylcyclopropyl)pyrimidin-2-yl]phenyl]-1-(5-methylpyrazin-2-yl)urea,
1-[4-[4-[(3S)-3-methylmorpholin-4-yl]-6-(1-methylsulfonylcyclopropyl)pyrimidin-2-yl]phenyl]-3-(1,3-oxazol-2-yl)urea,
3-(3-hydroxypropyl)-1-[4-[4-[(3S)-3-methylmorpholin-4-yl]-6-(1-methylsulfonylcyclopropyl)pyrimidin-2-yl]phenyl]urea,
1-[4-[4-[1-(benzenesulfonyl)cyclopropyl]-6-[(3S)-3-methylmorpholin-4-yl]pyrimidin-2-yl]phenyl]-3-cyclopropylurea,
1-[4-[4-[1-(benzenesulfonyl)cyclopropyl]-6-[(3S)-3-methylmorpholin-4-yl]pyrimidin-2-yl]phenyl]-3-cyclobutylurea,
1-[4-[4-[1-(benzenesulfonyl)cyclopropyl]-6-[(3S)-3-methylmorpholin-4-yl]pyrimidin-2-yl]phenyl]-3-(3,3,3-trifluoro-2-hydroxypropyl)urea,
1-[4-[4-[1-(benzenesulfonyl)cyclopropyl]-6-[(3S)-3-methylmorpholin-4-yl]pyrimidin-2-yl]phenyl]-3-propan-2-ylurea,
3-[4-[4-[1-(benzenesulfonyl)cyclopropyl]-6-[(3S)-3-methylmorpholin-4-yl]pyrimidin-2-yl]phenyl]-1-ethylurea,
1-[4-[4-[1-(benzenesulfonyl)cyclopropyl]-6-[(3S)-3-methylmorpholin-4-yl]pyrimidin-2-yl]phenyl]-3-(2-hydroxyethyl)urea,
3-[4-[4-[1-(benzenesulfonyl)cyclopropyl]-6-[(3S)-3-methylmorpholin-4-yl]pyrimidin-2-yl]phenyl]-1-propylurea,
1-[4-[4-[1-(benzenesulfonyl)cyclopropyl]-6-[(3S)-3-methylmorpholin-4-yl]pyrimidin-2-yl]phenyl]-3-methylurea,
1-[4-[4-[1-(benzenesulfonyl)cyclopropyl]-6-[(3S)-3-methylmorpholin-4-yl]pyrimidin-2-yl]phenyl]-3-(1-hydroxy-2-methylpropan-2-yl)urea,
1-[4-[4-[1-(benzenesulfonyl)cyclopropyl]-6-[(3S)-3-methylmorpholin-4-yl]pyrimidin-2-yl]phenyl]-3-(3-hydroxypropyl)urea,
1-[4-[4-[1-(benzenesulfonyl)cyclopropyl]-6-[(3S)-3-methylmorpholin-4-yl]pyrimidin-2-yl]phenyl]-3-(2-cyanoethyl)urea,
1-[4-[4-[1-(benzenesulfonyl)cyclopropyl]-6-[(3S)-3-methylmorpholin-4-yl]pyrimidin-2-yl]phenyl]-3-(1H-imidazol-2-ylmethyl)urea,
1-[4-[4-[1-(benzenesulfonyl)cyclopropyl]-6-[(3S)-3-methylmorpholin-4-yl]pyrimidin-2-yl]phenyl]-3-[1-(hydroxymethyl)cyclopropyl]urea,
1-[4-[4-[1-(benzenesulfonyl)cyclopropyl]-6-[(3S)-3-methylmorpholin-4-yl]pyrimidin-2-yl]phenyl]-3-(oxetan-3-yl)urea,
1-[4-[4-[1-(benzenesulfonyl)cyclopropyl]-6-[(3S)-3-methylmorpholin-4-yl]pyrimidin-2-yl]phenyl]-3-(1,2,4-thiadiazol-5-yl)urea,
1-[4-[4-[1-(benzenesulfonyl)cyclopropyl]-6-[(3S)-3-methylmorpholin-4-yl]pyrimidin-2-yl]phenyl]-3-(1-methylpyrazol-4-yl)urea,
1-[4-[4-[1-(benzenesulfonyl)cyclobutyl]-6-[(3S)-3-methylmorpholin-4-yl]pyrimidin-2-yl]phenyl]-3-cyclopropylurea,
1-[4-[4-[1-(benzenesulfonyl)cyclobutyl]-6-[(3S)-3-methylmorpholin-4-yl]pyrimidin-2-yl]phenyl]-3-(2-hydroxyethyl)urea,
1-[4-[4-[1-(benzenesulfonyl)cyclobutyl]-6-[(3S)-3-methylmorpholin-4-yl]pyrimidin-2-yl]phenyl]-3-methylurea,
1-[4-[4-[1-(benzenesulfonyl)cyclobutyl]-6-[(3S)-3-methylmorpholin-4-yl]pyrimidin-2-yl]phenyl]-3-(1-methylpyrazol-4-yl)urea,
1-[4-[4-[1-(benzenesulfonyl)cyclopentyl]-6-[(3S)-3-methylmorpholin-4-yl]pyrimidin-2-yl]phenyl]-3-cyclopropylurea,
1-[4-[4-[1-(benzenesulfonyl)cyclopentyl]-6-[(3S)-3-methylmorpholin-4-yl]pyrimidin-2-yl]phenyl]-3-(2-hydroxyethyl)urea,
1-[4-[4-[1-(benzenesulfonyl)cyclopentyl]-6-[(3S)-3-methylmorpholin-4-yl]pyrimidin-2-yl]phenyl]-3-(3-hydroxypropyl)urea,
1-[4-[4-[1-(benzenesulfonyl)cyclopentyl]-6-[(3S)-3-methylmorpholin-4-yl]pyrimidin-2-yl]phenyl]-3-(2-cyanoethyl)urea,
1-[4-[4-[4-(3-hydroxypropylsulfonyl)oxan-4-yl]-6-[(3S)-3-methylmorpholin-4-yl]pyrimidin-2-yl]phenyl]-3-methylurea,
1-ethyl-3-[4-[4-[4-(3-hydroxypropylsulfonyl)oxan-4-yl]-6-[(3S)-3-methylmorpholin-4-yl]pyrimidin-2-yl]phenyl]urea,
3-cyclopropyl-1-[4-[4-[4-(3-hydroxypropylsulfonyl)oxan-4-yl]-6-[(3S)-3-methylmorpholin-4-yl]pyrimidin-2-yl]phenyl]urea,
3-cyclobutyl-1-[4-[4-[4-(3-hydroxypropylsulfonyl)oxan-4-yl]-6-[(3S)-3-methylmorpholin-4-yl]pyrimidin-2-yl]phenyl]urea,
3-(2-cyanoethyl)-1-[4-[4-[4-(3-hydroxypropylsulfonyl)oxan-4-yl]-6-[(3S)-3-methylmorpholin-4-yl]pyrimidin-2-yl]phenyl]urea,
3-[4-[4-[4-(3-hydroxypropylsulfonyl)oxan-4-yl]-6-[(3S)-3-methylmorpholin-4-yl]pyrimidin-2-yl]phenyl]-1-propylurea,
3-(2-hydroxyethyl)-1-[4-[4-[4-(3-hydroxypropylsulfonyl)oxan-4-yl]-6-[(3S)-3-methylmorpholin-4-yl]pyrimidin-2-yl]phenyl]urea,
1-[4-[4-[4-(3-hydroxypropylsulfonyl)oxan-4-yl]-6-[(3S)-3-methylmorpholin-4-yl]pyrimidin-2-yl]phenyl]-3-(1-methylpyrazol-4-yl)urea,
1-[4-[4-[4-(3-hydroxypropylsulfonyl)oxan-4-yl]-6-[(3S)-3-methylmorpholin-4-yl]pyrimidin-2-yl]phenyl]-3-(1,3-thiazol-2-yl)urea,
1-[4-[4-[1-(3-hydroxypropylsulfonyl)cyclopropyl]-6-[(3S)-3-methylmorpholin-4-yl]pyrimidin-2-yl]phenyl]-3-(1,3-thiazol-2-yl)urea,
1-cyclopropyl-3-[4-[4-[1-(3-hydroxypropylsulfonyl)cyclopropyl]-6-[(3S)-3-methylmorpholin-4-yl]pyrimidin-2-yl]phenyl]-1-methylurea,
1-[4-[4-[1-(3-hydroxypropylsulfonyl)cyclopropyl]-6-[(3S)-3-methylmorpholin-4-yl]pyrimidin-2-yl]phenyl]-3-(1-methylcyclopropyl)urea,
3-(2,2-difluoroethyl)-1-[4-[4-[1-(3-hydroxypropylsulfonyl)cyclopropyl]-6-[(3S)-3-methylmorpholin-4-yl]pyrimidin-2-yl]phenyl]urea,
3-tert-butyl-1-[4-[4-[1-(3-hydroxypropylsulfonyl)cyclopropyl]-6-[(3S)-3-methylmorpholin-4-yl]pyrimidin-2-yl]phenyl]urea,
3-cyano-1-[4-[4-[1-(3-hydroxypropylsulfonyl)cyclopropyl]-6-[(3S)-3-methylmorpholin-4-yl]pyrimidin-2-yl]phenyl]urea,
3-hydroxy-N-[4-[4-[1-(3-hydroxypropylsulfonyl)cyclopropyl]-6-[(3S)-3-methylmorpholin-4-yl]pyrimidin-2-yl]phenyl]pyrrolidine-1-carboxamide,
1-[4-[4-[1-(3-hydroxypropylsulfonyl)cyclopropyl]-6-[(3S)-3-methylmorpholin-4-yl]pyrimidin-2-yl]phenyl]-3-(2-methylsulfonylethyl)urea,

3-(1,1-dioxothiolan-3-yl)-1-[4-[4-[1-(3-hydroxypropylsulfonyl)cyclopropyl]-6-[(3S)-3-methylmorpholin-4-yl]pyrimidin-2-yl]phenyl]urea,
2-[[4-[4-[1-(3-hydroxypropylsulfonyl)cyclopropyl]-6-[(3S)-3-methylmorpholin-4-yl]pyrimidin-2-yl]phenyl]carbamoylamino]-N,N-dimethylacetamide,
1-[4-[4-[(3S)-3-methylmorpholin-4-yl]-6-(1-pyridin-2-ylsulfonylcyclopropyl)pyrimidin-2-yl]phenyl]-3-(1,2,4-thiadiazol-5-yl)urea,
3-(1H-imidazol-2-ylmethyl)-1-[4-[4-[(3S)-3-methylmorpholin-4-yl]-6-(1-pyridin-2-ylsulfonylcyclopropyl)pyrimidin-2-yl]phenyl]urea,
3-[1-(hydroxymethyl)cyclopropyl]-1-[4-[4-[(3S)-3-methylmorpholin-4-yl]-6-(1-pyridin-2-ylsulfonylcyclopropyl)pyrimidin-2-yl]phenyl]urea,
3-(1H-imidazol-2-ylmethyl)-1-[4-[4-[(3S)-3-methylmorpholin-4-yl]-6-(1-pyridin-4-ylsulfonylcyclopropyl)pyrimidin-2-yl]phenyl]urea,
3-[1-(hydroxymethyl)cyclopropyl]-1-[4-[4-[(3S)-3-methylmorpholin-4-yl]-6-(1-pyridin-4-ylsulfonylcyclopropyl)pyrimidin-2-yl]phenyl]urea,
1-[4-[4-[4-(benzenesulfonyl)oxan-4-yl]-6-[(3S)-3-methylmorpholin-4-yl]pyrimidin-2-yl]phenyl]-3-methylurea,
3-[4-[4-[4-(benzenesulfonyl)oxan-4-yl]-6-[(3S)-3-methylmorpholin-4-yl]pyrimidin-2-yl]phenyl]-1-ethylurea,
1-[4-[4-[4-(benzenesulfonyl)oxan-4-yl]-6-[(3S)-3-methylmorpholin-4-yl]pyrimidin-2-yl]phenyl]-3-cyclopropylurea,
1-[4-[4-[4-(benzenesulfonyl)oxan-4-yl]-6-[(3S)-3-methylmorpholin-4-yl]pyrimidin-2-yl]phenyl]-3-(2-hydroxyethyl)urea,
1-[4-[4-[1-(benzenesulfonyl)cyclopropyl]-6-morpholin-4-ylpyrimidin-2-yl]phenyl]-3-cyclopropylurea,
3-[4-[4-[1-(benzenesulfonyl)cyclopropyl]-6-morpholin-4-ylpyrimidin-2-yl]phenyl]-1-ethylurea,
1-[4-[4-[1-(benzenesulfonyl)cyclopropyl]-6-morpholin-4-ylpyrimidin-2-yl]phenyl]-3-(2-hydroxyethyl)urea,
3-[4-[4-[1-(benzenesulfonyl)cyclopropyl]-6-morpholin-4-ylpyrimidin-2-yl]phenyl]-1-methylurea,
1-[4-[4-[1-(benzenesulfonyl)cyclopropyl]-6-morpholin-4-ylpyrimidin-2-yl]phenyl]-3-(3-hydroxypropyl)urea,
1-[4-[4-[1-(benzenesulfonyl)cyclopropyl]-6-morpholin-4-ylpyrimidin-2-yl]phenyl]-3-(2-cyanoethyl)urea,
1-[4-[4-[1-(benzenesulfonyl)cyclopropyl]-6-morpholin-4-ylpyrimidin-2-yl]phenyl]-3-(1,2,4-thiadiazol-5-yl)urea,
3-[4-[4-[1-(benzenesulfonyl)cyclopropyl]-6-morpholin-4-ylpyrimidin-2-yl]phenyl]-1-(1-methylpyrazol-4-yl)urea,
3-cyclopropyl-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea,
3-(2-hydroxyethyl)-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea,
3-(2-cyanoethyl)-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea,
3-(3-hydroxypropyl)-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea,
N-[4-[4-[1-(benzenesulfonyl)cyclopropyl]-6-[(3S)-3-methylmorpholin-4-yl]pyrimidin-2-yl]phenyl]-3-hydroxypyrrolidine-1-carboxamide,
2-[[4-[4-[1-(benzenesulfonyl)cyclopropyl]-6-[(3S)-3-methylmorpholin-4-yl]pyrimidin-2-yl]phenyl]carbamoylamino]acetic acid,
2-[[4-[4-[1-(benzenesulfonyl)cyclopropyl]-6-[(3S)-3-methylmorpholin-4-yl]pyrimidin-2-yl]phenyl]carbamoylamino]-N,N-dimethylacetamide,
3-[4-[4-[1-(benzenesulfonyl)cyclopropyl]-6-[(3S)-3-methylmorpholin-4-yl]pyrimidin-2-yl]phenyl]-1-cyclopropyl-1-methylurea,
1-[4-[4-[1-(benzenesulfonyl)cyclopropyl]-6-[(3S)-3-methylmorpholin-4-yl]pyrimidin-2-yl]phenyl]-3-(2-fluoroethyl)urea,
1-[4-[4-[1-(benzenesulfonyl)cyclobutyl]-6-[(3S)-3-methylmorpholin-4-yl]pyrimidin-2-yl]phenyl]-3-(2-cyanoethyl)urea,
1-[4-[4-[1-(benzenesulfonyl)cyclobutyl]-6-[(3S)-3-methylmorpholin-4-yl]pyrimidin-2-yl]phenyl]-3-(1H-imidazol-2-ylmethyl)urea,
1-[4-[4-[1-(benzenesulfonyl)cyclopropyl]-6-[(3S)-3-methylmorpholin-4-yl]pyrimidin-2-yl]phenyl]-3-(1-methylpyrazol-3-yl)urea,
3-[4-[4-[1-(benzenesulfonyl)cyclopropyl]-6-[(3S)-3-methylmorpholin-4-yl]pyrimidin-2-yl]phenyl]-1-(5-methyl-1,2-oxazol-3-yl)urea,
1-[4-[4-[1-(benzenesulfonyl)cyclopropyl]-6-[(3S)-3-methylmorpholin-4-yl]pyrimidin-2-yl]phenyl]-3-(2,2-difluoroethyl)urea,
1-[4-[4-[1-(benzenesulfonyl)cyclopropyl]-6-[(3S)-3-methylmorpholin-4-yl]pyrimidin-2-yl]phenyl]-3-(1,3-oxazol-2-yl)urea,
1-[4-[4-[1-(benzenesulfonyl)cyclopropyl]-6-[(3S)-3-methylmorpholin-4-yl]pyrimidin-2-yl]phenyl]-3-(1,2-oxazol-3-yl)urea,
1-[4-[4-[1-(benzenesulfonyl)cyclopropyl]-6-[(3S)-3-methylmorpholin-4-yl]pyrimidin-2-yl]phenyl]-3-(2,2,2-trifluoroethyl)urea,
1-[4-[4-[1-(benzenesulfonyl)cyclopropyl]-6-[(3S)-3-methylmorpholin-4-yl]pyrimidin-2-yl]phenyl]-3-(1-methylimidazol-4-yl)urea,
1-[4-[4-[1-(benzenesulfonyl)cyclopropyl]-6-[(3S)-3-methylmorpholin-4-yl]pyrimidin-2-yl]phenyl]-3-[(2S)-1-hydroxypropan-2-yl]urea,
1-[4-[4-[1-(benzenesulfonyl)cyclopropyl]-6-[(3S)-3-methylmorpholin-4-yl]pyrimidin-2-yl]phenyl]-3-[(2R)-1-hydroxypropan-2-yl]urea,
1-[4-[4-[1-(benzenesulfonyl)cyclopropyl]-6-[(3S)-3-methylmorpholin-4-yl]pyrimidin-2-yl]phenyl]-3-(2-chloroethyl)urea,
1-[4-[4-(1-ethylsulfonylcyclopropyl)-6-[(3S)-3-methylmorpholin-4-yl]pyrimidin-2-yl]phenyl]-3-(2-fluoroethyl)urea,
3-(2,2-difluoroethyl)-1-[4-[4-(1-ethylsulfonylcyclopropyl)-6-[(3S)-3-methylmorpholin-4-yl]pyrimidin-2-yl]phenyl]urea,
1-[4-[4-(1-cyclopropylsulfonylcyclopropyl)-6-[(3S)-3-methylmorpholin-4-yl]pyrimidin-2-yl]phenyl]-3-methylthiourea,
3-cyclopropyl-1-[4-[4-(1-cyclopropylsulfonylcyclopropyl)-6-[(3S)-3-methylmorpholin-4-yl]pyrimidin-2-yl]phenyl]thiourea,
1-[4-[4-(1-cyclopropylsulfonylcyclopropyl)-6-[(3S)-3-methylmorpholin-4-yl]pyrimidin-2-yl]phenyl]-3-(2-hydroxyethyl)thiourea,
3-cyclobutyl-1-[4-[4-[1-(3-hydroxypropylsulfonyl)cyclopropyl]-6-[(3S)-3-methylmorpholin-4-yl]pyrimidin-2-yl]phenyl]thiourea,
3-(2-cyanoethyl)-1-[4-[4-[1-(3-hydroxypropylsulfonyl)cyclopropyl]-6-[(3S)-3-methylmorpholin-4-yl]pyrimidin-2-yl]phenyl]thiourea,
3-(3-hydroxypropyl)-1-[4-[4-[1-(3-hydroxypropylsulfonyl)cyclopropyl]-6-[(3S)-3-methylmorpholin-4-yl]pyrimidin-2-yl]phenyl]thiourea,
1-[4-[4-[1-(3-hydroxypropylsulfonyl)cyclopropyl]-6-[(3S)-3-methylmorpholin-4-yl]pyrimidin-2-yl]phenyl]-3-methylthiourea,
3-cyclopropyl-1-[4-[4-[1-(3-hydroxypropylsulfonyl)cyclopropyl]-6-[(3S)-3-methylmorpholin-4-yl]pyrimidin-2-yl]phenyl]thiourea,
3-(2-hydroxyethyl)-1-[4-[4-[1-(3-hydroxypropylsulfonyl)cyclopropyl]-6-[(3S)-3-methylmorpholin-4-yl]pyrimidin-2-yl]phenyl]thiourea,
1-[4-[4-[1-(3-hydroxypropylsulfonyl)cyclopropyl]-6-[(3S)-3-methylmorpholin-4-yl]pyrimidin-2-yl]phenyl]-3-(1-methylpyrazol-4-yl)thiourea,
1-ethyl-3-[4-[4-[1-(3-hydroxypropylsulfonyl)cyclopropyl]-6-[(3S)-3-methylmorpholin-4-yl]pyrimidin-2-yl]phenyl]thiourea,
3-[4-[4-[1-(3-hydroxypropylsulfonyl)cyclopropyl]-6-[(3S)-3-methylmorpholin-4-yl pyrimidin-2-yl]phenyl]-1-propylthiourea,
3-(3-hydroxypropyl)-1-[4-[4-[(3S)-3-methylmorpholin-4-yl]-6-(1-methylsulfonylcyclopropyl)pyrimidin-2-yl]phenyl]thiourea,
1-[4-[4-[(3S)-3-methylmorpholin-4-yl]-6-(1-methylsulfonylcyclopropyl)pyrimidin-2-yl]phenyl]-3-propylthiourea,
1-[4-[4-[(3S)-3-methylmorpholin-4-yl]-6-(1-methylsulfonylcyclopropyl)pyrimidin-2-yl]phenyl]-3-(1-methylpyrazol-4-yl)thiourea,
3-methyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]thiourea,
3-cyclopropyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]thiourea,
3-(2-hydroxyethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]thiourea,
1-[4-[4-[1-(4-fluorophenyl)sulfonylcyclopropyl]-6-[(3S)-3-methylmorpholin-4-yl]pyrimidin-2-yl]phenyl]-3-methylthiourea,
3-cyclopropyl-1-[4-[4-[1-(4-fluorophenyl)sulfonylcyclopropyl]-6-[(3S)-3-methylmorpholin-4-yl]pyrimidin-2-yl]phenyl]thiourea,
1-[4-[4-[1-(4-fluorophenyl)sulfonylcyclopropyl]-6-[(3S)-3-methylmorpholin-4-yl]pyrimidin-2-yl]phenyl]-3-(2-hydroxyethyl)thiourea,
1-[4-[4-[1-(benzenesulfonyl)cyclopropyl]-6-[(3S)-3-methylmorpholin-4-yl]pyrimidin-2-yl]phenyl]-3-cyclopropylthiourea,
1-[4-[4-[1-(benzenesulfonyl)cyclopropyl]-6-[(3S)-3-methylmorpholin-4-yl]pyrimidin-2-yl]phenyl]-3-(1-methylpyrazol-4-yl)thiourea,
1-[4-[4-[1-(benzenesulfonyl)cyclopropyl]-6-[(3S)-3-methylmorpholin-4-yl]pyrimidin-2-yl]phenyl]-3-(3-hydroxypropyl)thiourea,
1-[4-[4-[1-(benzenesulfonyl)cyclopropyl]-6-[(3S)-3-methylmorpholin-4-yl]pyrimidin-2-yl]phenyl]-3-(2-cyanoethyl)thiourea,
3-[4-[4-[1-(benzenesulfonyl)cyclopropyl]-6-[(3S)-3-methylmorpholin-4-yl]pyrimidin-2-yl]phenyl]-1-ethylthiourea,
1-[4-[4-[1-(benzenesulfonyl)cyclopropyl]-6-[(3S)-3-methylmorpholin-4-yl]pyrimidin-2-yl]phenyl]-3-(2-hydroxyethyl)thiourea,
1-[4-[4-[1-(benzenesulfonyl)cyclopropyl]-6-[(3S)-3-methylmorpholin-4-yl]pyrimidin-2-yl]phenyl]-3-methylthiourea,
1-[4-[4-[1-(benzenesulfonyl)cyclopropyl]-6-[(3S)-3-methylmorpholin-4-yl]pyrimidin-2-yl]phenyl]-3-(1H-imidazol-2-ylmethyl)thiourea,
3-(2-hydroxyethyl)-1-[4-[4-[(3S)-3-methylmorpholin-4-yl]-6-(1-pyridin-4-ylsulfonylcyclopropyl)pyrimidin-2-yl]phenyl]thiourea,
3-cyclopropyl-1-[4-[4-[(3S)-3-methylmorpholin-4-yl]-6-(1-pyridin-4-ylsulfonylcyclopropyl)pyrimidin-2-yl]phenyl]thiourea,
3-methyl-1-[4-[4-[(3S)-3-methylmorpholin-4-yl]-6-(1-pyridin-4-ylsulfonylcyclopropyl)pyrimidin-2-yl]phenyl]thiourea,
1-[4-[4-[1-(benzenesulfonyl)cyclopropyl]-6-[(3S)-3-methylmorpholin-4-yl]pyrimidin-2-yl]phenyl]-3-(1-hydroxy-2-methylpropan-2-yl)thiourea,
3-cyclopropyl-1-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]urea,
3-methyl-1-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]urea,
3-cyclopropyl-1-[4-[4-[(3S)-3-methylmorpholin-4-yl]-6-[1-(1,3-thiazol-2-ylsulfonyl)cyclopropyl]pyrimidin-2-yl]phenyl]urea,
3-methyl-1-[4-[4-[(3S)-3-methylmorpholin-4-yl]-6-[1-(1,3-thiazol-2-ylsulfonyl)cyclopropyl]pyrimidin-2-yl]phenyl]urea,
1-[4-[4-[1-(1H-imidazol-2-ylsulfonyl)cyclopropyl]-6-[(3S)-3-methylmorpholin-4-yl]pyrimidin-2-yl]phenyl]-3-methylurea,
3-cyclopropyl-1-[4-[4-[1-(1H-imidazol-2-ylsulfonyl)cyclopropyl]-6-[(3S)-3-methylmorpholin-4-yl]pyrimidin-2-yl]phenyl]urea,
3-(2-cyanoethyl)-1-[4-[4-[(3S)-3-methylmorpholin-4-yl]-6-(1-methylsulfonylcyclopropyl)pyrimidin-2-yl]phenyl]thiourea,
3-cyclopropyl-1-[4-[4-[1-[(4-methyl-1,3-thiazol-2-yl)sulfonyl]cyclopropyl]-6-morpholin-4-ylpyrimidin-2-yl]phenyl]urea,
3-(2-hydroxyethyl)-1-[4-[4-[1-[(4-methyl-1,3-thiazol-2-yl)sulfonyl]cyclopropyl]-6-morpholin-4-ylpyrimidin-2-yl]phenyl]urea,
3-(1-methylpyrazol-4-yl)-1-[4-[4-[1-[(4-methyl-1,3-thiazol-2-yl)sulfonyl]cyclopropyl]-6-morpholin-4-ylpyrimidin-2-yl]phenyl]urea,
3-methyl-1-[4-[4-[1-[(4-methyl-1,3-thiazol-2-yl)sulfonyl]cyclopropyl]-6-morpholin-4-ylpyrimidin-2-yl]phenyl]urea,
1-ethyl-3-[4-[4-[1-[(4-methyl-1,3-thiazol-2-yl)sulfonyl]cyclopropyl]-6-morpholin-4-ylpyrimidin-2-yl]phenyl]urea,
3-cyclobutyl-1-[4-[4-[1-[(4-methyl-1,3-thiazol-2-yl)sulfonyl]cyclopropyl]-6-morpholin-4-ylpyrimidin-2-yl]phenyl]urea,
3-(2-cyanoethyl)-1-[4-[4-[1-[(4-methyl-1,3-thiazol-2-yl)sulfonyl]cyclopropyl]-6-morpholin-4-ylpyrimidin-2-yl]phenyl]urea,
3-[4-[4-[1-[(4-methyl-1,3-thiazol-2-yl)sulfonyl]cyclopropyl]-6-morpholin-4-ylpyrimidin-2-yl]phenyl]-1-propylurea,
1-[4-[4-[1-[(4-methyl-1,3-thiazol-2-yl)sulfonyl]cyclopropyl]-6-morpholin-4-ylpyrimidin2-yl]phenyl]-3-propan-2-ylurea,
3-cyclopropyl-1-[4-[4-[1-(3-hydroxypropylsulfonyl)cyclobutyl]-6-[(3S)-3-methylmorpholin-4-yl]pyrimidin-2-yl]phenyl]urea,
1-[4-[4-[1-(3-hydroxypropylsulfonyl)cyclobutyl]-6-[(3S)-3-methylmorpholin-4-yl]pyrimidin-2-yl]phenyl]-3-methylurea,
3-(2-hydroxyethyl)-1-[4-[4-[1-(3-hydroxypropylsulfonyl)cyclobutyl]-6-[(3S)-3-methylmorpholin-4-yl]pyrimidin-2-yl]phenyl]urea,
3-(2-cyanoethyl)-1-[4-[4-[1-(3-hydroxypropylsulfonyl)cyclobutyl]-6-[(3S)-3-methylmorpholin-4-yl]pyrimidin-2-yl]phenyl]urea,
1-[4-[4-[1-(3-hydroxypropylsulfonyl)cyclobutyl]-6-[(3S)-3-methylmorpholin-4-yl]pyrimidin-2-yl]phenyl]-3-(1-methylpyrazol-4-yl)urea,
1-[4-[4-[1-(3-hydroxypropylsulfonyl)cyclobutyl]-6-[(3S)-3-methylmorpholin-4-yl]pyrimidin-2-yl]phenyl]-3-(1H-imidazol-2-ylmethyl)urea,
3-cyclopropyl-1-[4-[4-[1-(3-hydroxypropylsulfonyl)cyclopentyl]-6-[(3S)-3-methylmorpholin-4-yl]pyrimidin-2-yl]phenyl]urea,
3-(2-hydroxyethyl)-1-[4-[4-[1-(3-hydroxypropylsulfonyl)cyclopentyl]-6-[(3S)-3-methylmorpholin-4-yl]pyrimidin-2-yl]phenyl]urea,
3-(2-cyanoethyl)-1-[4-[4-[1-(3-hydroxypropylsulfonyl)cyclopentyl]-6-[(3S)-3-methylmorpholin-4-yl]pyrimidin-2-yl]phenyl]urea,
3-(3-hydroxypropyl)-1-[4-[4-[1-(3-hydroxypropylsulfonyl)cyclopentyl]-6-[(3-methylmorpholin-4-yl]pyrimidin-2-yl]phenyl]urea,
1-ethyl-3-[4-[4-[1-(3-hydroxypropylsulfonyl)cyclobutyl]-6-[(3S)-3-methylmorpholin-4-yl]pyrimidin-2-yl]phenyl]urea,
3-[4-[4-[1-(3-hydroxypropylsulfonyl)cyclobutyl]-6-[(3S)-3-methylmorpholin-4-yl]pyrimidin-2-yl]phenyl]-1-propylurea,
3-(3-hydroxypropyl)-1-[4-[4-[1-(3-hydroxypropylsulfonyl)cyclobutyl]-6-[(3S)-3-methylmorpholin-4-yl]pyrimidin-2-yl]phenyl]urea,
1-[4-[4-[1-(3-hydroxypropylsulfonyl)cyclobutyl]-6-[(3S)-3-methylmorpholin-4-yl]pyrimidin-2-yl]phenyl]-3-(1,2-oxazol-3-yl)urea,
3-(2-fluoroethyl)-1-[4-[4-[1-(3-hydroxypropylsulfonyl)cyclobutyl]-6-[(3S)-3-methylmorpholin-4-yl]pyrimidin-2-yl]phenyl]urea,
3-(2,2-difluoroethyl)-1-[4-[4-[1-(3-hydroxypropylsulfonyl)cyclobutyl]-6-[(3S)-3-methylmorpholin-4-yl]pyrimidin-2-yl]phenyl]urea,
3-(1-hydroxy-2-methylpropan-2-yl)-1-[4-[4-[1-(3-hydroxypropylsulfonyl)cyclobutyl]-6-(3S)-3-methylmorpholin-4-yl]pyrimidin-2-yl]phenyl]urea,
3-[(2S)-1-hydroxypropan-2-yl]-1-[4-[4-[1-(3-hydroxypropylsulfonyl)cyclobutyl]-6-[(3S)-3-methylmorpholin-4-yl]pyrimidin-2-yl]phenyl]urea,
3-[(2R)-1-hydroxypropan-2-yl]-1-[4-[4-[1-(3-hydroxypropylsulfonyl)cyclobutyl]-6-[(3S)-3-methylmorpholin-4-yl]pyrimidin-2-yl]phenyl]urea,
1-[4-[4-[1-(3-hydroxypropylsulfonyl)cyclobutyl]-6-[(3S)-3-methylmorpholin-4-yl]pyrimidin-2-yl]phenyl]-3-(1,2,4-thiadiazol-5-yl)urea,
1-[4-[4-[1-(3-hydroxypropylsulfonyl)cyclobutyl]-6-[(3S)-3-methylmorpholin-4-yl]pyrimidin-2-yl]phenyl]-3-(1,3-thiazol-2-yl)urea,
N-[2-[1-[2-[4-(cyclopropylcarbamoylamino)phenyl]-6-[(3S)-3-methylmorpholin-4-yl]pyrimidin-4-yl]cyclopropyl]sulfonylethyl]acetamide,
N-[2-[1-[2-[4-(ethylcarbamoylamino)phenyl]-6-[(3S)-3-methylmorpholin-4-yl]pyrimidin-4-yl]cyclopropyl]sulfonylethyl]acetamide,
2-[1-[2-[4-(cyclopropylcarbamoylamino)phenyl]-6-morpholin-4-ylpyrimidin-4-yl]cyclopropyl]sulfonylacetamide,
2-[1-[2-[4-(cyclopropylcarbamoylamino)phenyl]-6-morpholin-4-ylpyrimidin-4-yl]cyclopropyl]sulfonyl-N-methylacetamide,
3-cyclopropyl-1-[4-[4-[1-(2-hydroxyethylsulfonyl)cyclopropyl]-6-morpholin-4-ylpyrimidin-2-yl]phenyl]urea,
1-[4-[4-[1-(2-hydroxyethylsulfonyl)cyclopropyl]-6-morpholin-4-ylpyrimidin-2-yl]phenyl]-3-methylurea,
3-ethyl-1-[4-[4-[1-(2-hydroxyethylsulfonyl)cyclopropyl]-6-morpholin-4-ylpyrimidin-2-yl]phenyl]urea,
1-[4-[4-[1-(5-fluoropyridin-2-yl)sulfonylcyclopropyl]-6-[(3S)-3-methylmorpholin-4-yl]pyrimidin-2-yl]phenyl]-3-methylurea,
3-cyclopropyl-1-[4-[4-[1-(5-fluoropyridin-2-yl)sulfonylcyclopropyl]-6-[(3S)-3-methylmorpholin-4-yl]pyrimidin-2-yl]phenyl]urea,
N,N-dimethyl-6-[1-[2-[4-(methylcarbamoylamino)phenyl]-6-[(3S)-3-methylmorpholin-4-yl]pyrimidin-4-yl]cyclopropyl]sulfonylpyridine-3-carboxamide,
6-[1-[2-[4-(cyclopropylcarbamoylamino)phenyl]-6-[(3S)-3-methylmorpholin-4-yl]pyrimidin-4-yl]cyclopropyl]sulfonyl-N,N-dimethylpyridine-3-carboxamide,
N,N-dimethyl-3-[1-[2-[4-(methylcarbamoylamino)phenyl]-6-[(3S)-3-methylmorpholin-4-yl]pyrimidin-4-yl]cyclopropyl]sulfonylpyridine-2-carboxamide,
3-[1-[2-[4-(cyclopropylcarbamoylamino)phenyl]-6-[(3S)-3-methylmorpholin-4-yl]pyrimidin-4-yl]cyclopropyl]sulfonyl-N,N-dimethylpyridine-2-carboxamide,
1-[4-[4-[1-(2-methoxyethylsulfonyl)cyclopropyl]-6-[(3S)-3-methylmorpholin-4-yl]pyrimidin-2-yl]phenyl]-3-methylurea,
3-cyclopropyl-1-[2-fluoro-4-[4-[(3S)-3-methylmorpholin-4-yl]-6-(1-methylsulfonylcyclopropyl)pyrimidin-2-yl]phenyl]urea,
1-[2-fluoro-4-[4-[(3S)-3-methylmorpholin-4-yl]-6-(1-methylsulfonylcyclopropyl)pyrimidin-2-yl]phenyl]-3-methylurea,
3-ethyl-1-[2-fluoro-4-[4-[(3S)-3-methylmorpholin-4-yl]-6-(1-methylsulfonylcyclopropyl)pyrimidin-2-yl]phenyl]urea,
1-[2-fluoro-4-[4-[(3S)-3-methylmorpholin-4-yl]-6-(1-methylsulfonylcyclopropyl)pyrimidin-2-yl]phenyl]-3-(2-hydroxyethyl)urea,
1-[2-fluoro-4-[4-[(3S)-3-methylmorpholin-4-yl]-6-(1-methylsulfonylcyclopropyl)pyrimidin-2-yl]phenyl]-3-(1-methylpyrazol-4-yl)urea,
1-[4-[4-[1-(3-fluorophenyl)sulfonylcyclopropyl]-6-[(3S)-3-methylmorpholin-4-yl]pyrimidin-2-yl]phenyl]-3-methylurea,
3-cyclopropyl-1-[4-[4-[1-(3-fluorophenyl)sulfonylcyclopropyl]-6-[(3S)-3-methylmorpholin-4-yl]pyrimidin-2-yl]phenyl]urea,
1-[4-[4-[1-(3-fluorophenyl)sulfonylcyclopropyl]-6-[(3S)-3-methylmorpholin-4-yl]pyrimidin-2-yl]phenyl]-3-(2-hydroxyethyl)urea,
3-(2-fluoroethyl)-1-[4-[4-[1-(3-fluorophenyl)sulfonylcyclopropyl]-6-[(3S)-3-methylmorpholin-4-yl]pyrimidin-2-yl]phenyl]urea,
3-(2,2-difluoroethyl)-1-[4-[4-[1-(3-fluorophenyl)sulfonylcyclopropyl]-6-[(3S)-3-methylmorpholin-4-yl]pyrimidin-2-yl]phenyl]urea,
1-ethyl-3-[4-[4-[1-(3-fluorophenyl)sulfonylcyclopropyl]-6-[(3S)-3-methylmorpholin-4-yl]pyrimidin-2-yl]phenyl]urea,
1-[4-[4-[1-(3-fluorophenyl)sulfonylcyclopropyl]-6-[(3S)-3-methylmorpholin-4-yl]pyrimidin-2-yl]phenyl]-3-(1-methylpyrazol-4-yl)urea,
3-methyl-1-[4-[4-[(3S)-3-methylmorpholin-4-yl]-6-[1-(2-methylphenyl)sulfonylcyclopropyl]pyrimidin-2-yl]phenyl]urea,
3-cyclopropyl-1-[4-[4-[(3S)-3-methylmorpholin-4-yl]-6-[1-(2-methylphenyl)sulfonylcyclopropyl]pyrimidin-2-yl]phenyl]urea,
3-(2-hydroxyethyl)-1-[4-[4-[(3S)-3-methylmorpholin-4-yl]-6-[1-(2-methylphenyl)sulfonylcyclopropyl]pyrimidin-2-yl]phenyl]urea,
3-(2-fluoroethyl)-1-[4-[4-[(3S)-3-methylmorpholin-4-yl]-6-[1-(2-methylphenyl)sulfonylcyclopropyl]pyrimidin-2-yl]phenyl]urea,
3-(2,2-difluoroethyl)-1-[4-[4-[(3S)-3-methylmorpholin-4-yl]-6-[1-(2-methylphenyl)sulfonylcyclopropyl]pyrimidin-2-yl]phenyl]urea,
1-ethyl-3-[4-[4-[(3S)-3-methylmorpholin-4-yl]-6-[1-(2-methylphenyl)sulfonylcyclopropyl]pyrimidin-2-yl]phenyl]urea,
1-[4-[4-[(3S)-3-methylmorpholin-4-yl]-6-[1-(2-methylphenyl)sulfonylcyclopropyl]pyrimidin-2-yl]phenyl]-3-(1-methylpyrazol-4-yl)urea,
1-[4-[4-[1-(1,3-dimethylpyrazol-4-yl)sulfonylcyclopropyl]-6-[(3S)-3-methylmorpholin-4-yl]pyrimidin-2-yl]phenyl]-3-methylurea,
3-cyclopropyl-1-[4-[4-[1-(1,3-dimethylpyrazol-4-yl)sulfonylcyclopropyl]-6-[(3S)-3-methylmorpholin-4-yl]pyrimidin-2-yl]phenyl]urea,
1-[4-[4-[1-(1,3-dimethylpyrazol-4-yl)sulfonylcyclopropyl]-6-[(3S)-3-methylmorpholin-4-yl]pyrimidin-2-yl]phenyl]-3-(2-hydroxyethyl)urea,
1-[4-[4-[1-(1,3-dimethylpyrazol-4-yl)sulfonylcyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-(1,3-dimethylpyrazol-4-yl)sulfonylcyclopropyl]-6-[(3S)-3-methylmorpholin-4-yl]pyrimidin-2-yl]phenyl]urea,
3-[4-[4-[1-(1,3-dimethylpyrazol-4-yl)sulfonylcyclopropyl]-6-[(3S)-3-methylmorpholin-4-yl]pyrimidin-2-yl]phenyl]-1-ethylurea,
1-[4-[4-[1-(1,3-dimethylpyrazol-4-yl)sulfonylcyclopropyl]-6-[(3S)-3-methylmorpholin-4-yl]pyrimidin-2-yl]phenyl]-3-(1-methylpyrazol-4-yl)urea,
3-cyclopropyl-1-[4-[4-morpholin-4-yl-6-(1-pyridin-2-ylsulfonylcyclopropyl)pyrimidin-2-yl]phenyl]urea,
3-methyl-1-[4-[4-morpholin-4-yl-6-(1-pyridin-2-ylsulfonylcyclopropyl)pyrimidin-2-yl]phenyl]urea,
3-ethyl-1-[4-[4-morpholin-4-yl-6-(1-pyridin-2-ylsulfonylcyclopropyl)pyrimidin-2-yl]phenyl]urea,
3-(2-hydroxyethyl)-1-[4-[4-morpholin-4-yl-6-(1-pyridin-2-ylsulfonylcyclopropyl)pyrimidin-2-yl]phenyl]urea,
3-(1-methylpyrazol-4-yl)-1-[4-[4-morpholin-4-yl-6-(1-pyridin-2-ylsulfonylcyclopropyl)pyrimidin-2-yl]phenyl]urea,
1-[4-[4-[1-(2-fluoro-4-methylaminophenyl)sulfonylcyclopropyl]-6-[(3S)-3-methylmorpholin-4-yl]pyrimidin-2-yl]phenyl]-3-methylurea,
3-cyclopropyl-1-[4-[4-[1-(2,4-difluorophenyl)sulfonylcyclopropyl]-6-[(3S)-3-methylmorpholin-4-yl]pyrimidin-2-yl]phenyl]urea,
1-[4-[4-[1-(2,4-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(2,4-difluorophenyl)sulfonylcyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-(2,4-difluorophenyl)sulfonylcyclopropyl]-6-[(3S)-3-methylmorpholin-4-yl]pyrimidin-2-yl]phenyl]urea,
3-[4-[4-[1-(2,4-difluorophenyl)sulfonylcyclopropyl]-6-[(3S)-3-methylmorpholin-4-yl]pyrimidin-2-yl]phenyl]-1-ethylurea,
1-[4-[4-[1-(2,4-difluorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-[1-(2,4-difluorophenyl)sulfonylcyclopropyl]-6-[(3S)-3-methylmorpholin-4-yl]pyrimidin-2-yl]phenyl]-3-[(1-hydroxycyclopropyl)methyl]urea,
1-[4-[4-[1-(2-fluorophenyl)sulfonylcyclopropyl]-6-[(3S)-3-methylmorpholin-4-yl]pyrimidin-2-yl]phenyl]-3-methylurea,
3-cyclopropyl-1-[4-[4-[1-(2-fluorophenyl)sulfonylcyclopropyl]-6-[(3S)-3-methylmorpholin-4-yl]pyrimidin-2-yl]phenyl]urea,
1-[4-[4-[1-(2-fluorophenyl)sulfonylcyclopropyl]-6-[(3S)-3-methylmorpholin-4-yl]pyrimidin-2-yl]phenyl]-3-(2-hydroxyethyl)urea,
3-(2-fluoroethyl)-1-[4-[4-[1-(2-fluorophenyl)sulfonylcyclopropyl]-6-[(3S)-3-methylmorpholin-4-yl]pyrimidin-2-yl]phenyl]urea,
3-(2,2-difluoroethyl)-1-[4-[4-[1-(2-fluorophenyl)sulfonylcyclopropyl]-6-[(3S)-3-methylmorpholin-4-yl]pyrimidin-2-yl]phenyl]urea,
1-ethyl-3-[4-[4-[1-(2-fluorophenyl)sulfonylcyclopropyl]-6-[(3S)-3-methylmorpholin-4-yl]pyrimidin-2-yl]phenyl]urea,
1-[4-[4-[1-(2-fluorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-[1-(2-fluorophenyl)sulfonylcyclopropyl]-6-[(3S)-3-methylmorpholin-4-yl]pyrimidin-2-yl]phenyl]-3-[(1-hydroxycyclopropyl)methyl]urea,
1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-methylurea,
3-cyclopropyl-1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]urea,
1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-(2-hydroxyethyl)urea,
1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]urea,
1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-[(1-hydroxycyclopropyl)methyl]urea,
3-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-1-ethylurea,
1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-(1-methylpyrazol-43-yl)urea,
1-[4-[4-[1-(2,5-difluorophenyl)sulfonylcyclopropyl]-6-[(3S)-3-methylmorpholin-4-yl]pyrimidin-2-yl]phenyl]-3-methylurea,
3-cyclopropyl-1-[4-[4-[1-(2,5-difluorophenyl)sulfonylcyclopropyl]-6-[(3S)-3-methylmorpholin-4-yl]pyrimidin-2-yl]phenyl]urea,
1-[4-[4-[1-(2,5-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(2,5-difluorophenyl)sulfonylcyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-(2,5-difluorophenyl)sulfonylcyclopropyl]-6-[(3S)-3-methylmorpholin-4-yl]pyrimidin-2-yl]phenyl]urea,
3-[4-[4-[1-(2,5-difluorophenyl)sulfonylcyclopropyl]-6-[(3S)-3-methylmorpholin-4-yl]pyrimidin-2-yl]phenyl]-1-ethylurea,
1-[4-[4-[1-(2,5-difluorophenyl)sulfonylcyclopropyl]-6-[(3S)-3-methylmorpholin-4-yl]pyrimidin-2-yl]phenyl]-3-(1-methylpyrazol-43-yl)urea
1-[4-[4-[1-(5-fluoro-2-methylphenyl)sulfonylcyclopropyl]-6-[(3S)-3-methylmorpholin-4-yl]pyrimidin-2-yl]phenyl]-3-methylurea,
3-cyclopropyl-1-[4-[4-[1-(5-fluoro-2-methylphenyl)sulfonylcyclopropyl]-6-[(3S)-3-methylmorpholin-4-yl]pyrimidin-2-yl]phenyl]urea,
1-[4-[4-[1-(5-fluoro-2-methylphenyl)sulfonylcyclopropyl]-6-[(3S)-3-methylmorpholin-4-yl]pyrimidin-2-yl]phenyl]-3-(2-hydroxyethyl)urea,
3-(2-fluoroethyl)-1-[4-[4-[1-(5-fluoro-2-methylphenyl)sulfonylcyclopropyl]-6-[(3S)-3-methylmorpholin-4-yl]pyrimidin-2-yl]phenyl]urea,
3-(2,2-difluoroethyl)-1-[4-[4-[1-(5-fluoro-2-methylphenyl)sulfonylcyclopropyl]-6-[(3S)-3-methylmorpholin-4-yl]pyrimidin-2-yl]phenyl]urea,
1-ethyl-3-[4-[4-[1-(5-fluoro-2-methylphenyl)sulfonylcyclopropyl]-6-[(3S)-3-methylmorpholin-4-yl]pyrimidin-2-yl]phenyl]urea,
1-[4-[4-[1-(5-fluoro-2-methylphenyl)sulfonylcyclopropyl]-6-[(3S)-3-methylmorpholin-4-yl]pyrimidin-2-yl]phenyl]-3-(1-methylpyrazol-43-yl)urea,
3-(2-hydroxyethyl)-1-[4-[4-[(3S)-3-methylmorpholin-4-yl]-6-(1-methylsulfonylcyclobutyl)pyrimidin-2-yl]phenyl]thiourea,
3-(1H-imidazol-2-ylmethyl)-1-[4-[4-[(3S)-3-methylmorpholin-4-yl]-6-(1-methylsulfonylcyclobutyl)pyrimidin-2-yl]phenyl]thiourea,
3-[(2S)-1-hydroxypropan-2-yl]-1-[4-[4-[(3S)-3-methylmorpholin-4-yl]-6-(1-methylsulfonylcyclobutyl)pyrimidin-2-yl]phenyl]thiourea,
3-[(2R)-1-hydroxypropan-2-yl]-1-[4-[4-[(3S)-3-methylmorpholin-4-yl]-6-(1-methylsulfonylcyclobutyl)pyrimidin-2-yl]phenyl]thiourea,
3-(3-hydroxypropyl)-1-[4-[4-[(3S)-3-methylmorpholin-4-yl]-6-(1-methylsulfonylcyclobutyl)pyrimidin-2-yl]phenyl]thiourea,
3-(1H-imidazol-2-ylmethyl)-1-[4-[4-[(3S)-3-methylmorpholin-4-yl]-6-(1-pyridin-4-ylsulfonylcyclobutyl)pyrimidin-2-yl]phenyl]thiourea,
3-cyclopropyl-1-[4-[4-[1-[4-(difluoromethoxy)phenyl]sulfonylcyclopropyl]-6-[(3S)-3-methylmorpholin-4-yl]pyrimidin-2-yl]phenyl]urea,
1-[4-[4-[1-[4-(difluoromethoxy)phenyl]sulfonylcyclopropyl]-6-[(3S)-3-methylmorpholin-4-yl]pyrimidin-2-yl]phenyl]-3-methylurea,
3-[4-[4-[1-[4-(difluoromethoxy)phenyl]sulfonylcyclopropyl]-6-[(3S)-3-methylmorpholin-4-yl]pyrimidin-2-yl]phenyl]-1-ethylurea,
1-[4-[4-[1-[4-(difluoromethoxy)phenyl]sulfonylcyclopropyl]-6-[(3S)-3-methylmorpholin-4-yl]pyrimidin-2-yl]phenyl]-3-(2-hydroxyethyl)urea,
1-[4-[4-[1-[4-(difluoromethoxy)phenyl]sulfonylcyclopropyl]-6-[(3S)-3-methylmorpholin-4-yl]pyrimidin-2-yl]phenyl]-3-(1-methylpyrazol-4-yl)urea,
1-[4-[4-[1-[4-(difluoromethoxy)phenyl]sulfonylcyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-[4-(difluoromethoxy)phenyl]sulfonylcyclopropyl]-6-[(3S)-3-methylmorpholin-4-yl]pyrimidin-2-yl]phenyl]urea,
1-[4-[4-[1-(3,4-difluorophenyl)sulfonylcyclopropyl]-6-[(3S)-3-methylmorpholin-4-yl]pyrimidin-2-yl]phenyl]-3-methylurea,
3-[4-[4-[1-(3,4-difluorophenyl)sulfonylcyclopropyl]-6-[(3S)-3-methylmorpholin-4-yl]pyrimidin-2-yl]phenyl]-1-ethylurea,
3-cyclopropyl-1-[4-[4-[1-(3,4-difluorophenyl)sulfonylcyclopropyl]-6-[(3S)-3-methylmorpholin-4-yl]pyrimidin-2-yl]phenyl]urea,
1-[4-[4-[1-(3,4-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(3,4-difluorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-[1-(3,4-difluorophenyl)sulfonylcyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-(3,4-difluorophenyl)sulfonylcyclopropyl]-6-[(3S)-3-methylmorpholin-4-yl]pyrimidin-2-yl]phenyl]urea,
1-[4-[4-[1-[3-fluoro-4-(2-hydroxyethylamino)phenyl]sulfonylcyclopropyl]-6-[(3S)-3-methylmorpholin-4-yl]pyrimidin-2-yl]phenyl]-3-(2-hydroxyethyl)urea,
1-[4-[4-[1-(benzenesulfonyl)cyclobutyl]-6-[(3S)-3-methylmorpholin-4-yl]pyrimidin-2-yl]phenyl]-3-(1-hydroxy-2-methylpropan-2-yl)urea,
1-[4-[4-[1-(benzenesulfonyl)cyclobutyl]-6-[(3S)-3-methylmorpholin-4-yl]pyrimidin-2-yl]phenyl]-3-[(2S)-1-hydroxypropan-2-yl]urea,
1-[4-[4-[1-(benzenesulfonyl)cyclobutyl]-6-[(3S)-3-methylmorpholin-4-yl]pyrimidin-2-yl]phenyl]-3-[(2R)-1-hydroxypropan-2-yl]urea,
1-[4-[4-[1-(benzenesulfonyl)cyclobutyl]-6-[(3S)-3-methylmorpholin-4-yl]pyrimidin-2-yl]phenyl]-3-(3-hydroxypropyl)urea,
1-[4-[4-[1-(benzenesulfonyl)cyclobutyl]-6-[(3S)-3-methylmorpholin-4-yl]pyrimidin-2-yl]phenyl]-3-(2-fluoroethyl)urea,
1-[4-[4-(1-cyclopropylsulfonylcyclopropyl)-6-[(3S)-3-methylmorpholin-4-yl]pyrimidin-2-yl]phenyl]-3-(2-fluoroethyl)urea,
1-[4-[4-(1-cyclopropylsulfonylcyclopropyl)-6-[(3S)-3-methylmorpholin-4-yl]pyrimidin-2-yl]phenyl]-3-(2,2-difluoroethyl)urea,
1-[4-[4-(1-cyclopropylsulfonylcyclopropyl)-6-[(3S)-3-methylmorpholin-4-yl]pyrimidin-2-yl]phenyl]-3-(1H-imidazol-2-ylmethyl)urea,
1-[4-[4-(1-cyclopropylsulfonylcyclobutyl)-6-[(3S)-3-methylmorpholin-4-yl]pyrimidin-2-yl]phenyl]-3-(1-hydroxy-2-methylpropan-2-yl)urea,
1-[4-[4-(1-cyclopropylsulfonylcyclobutyl)-6-[(3S)-3-methylmorpholin-4-yl]pyrimidin-2-yl]phenyl]-3-[(2S)-1-hydroxypropan-2-yl]urea,
1-[4-[4-(1-cyclopropylsulfonylcyclobutyl)-6-[(3S)-3-methylmorpholin-4-yl]pyrimidin-2-yl]phenyl]-3-[(2R)-1-hydroxypropan-2-yl]urea,
1-[4-[4-(1-cyclopropylsulfonylcyclobutyl)-6-[(3S)-3-methylmorpholin-4-yl]pyrimidin-2-yl]phenyl]-3-(3-hydroxypropyl)urea,
3-(2-fluoroethyl)-1-[4-[4-[(3S)-3-methylmorpholin-4-yl]-6-(1-methylsulfonylcyclopropyl)pyrimidin-2-yl]phenyl]urea,
3-(2,2-difluoroethyl)-1-[4-[4-[(3S)-3-methylmorpholin-4-yl]-6-(1-methylsulfonylcyclopropyl)pyrimidin-2-yl]phenyl]urea,
3-[(2S)-1-hydroxypropan-2-yl]-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-[(2R)-1-hydroxypropan-2-yl]-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-fluoroethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2,2-difluoroethyl)-1-[4-[4-[(3S)-3-methylmorpholin-4-yl]-6-(1-pyridin-2-ylsulfonylcyclopropyl)pyrimidin-2-yl]phenyl]urea,
1-[4-[4-[(3S)-3-methylmorpholin-4-yl]-6-(1-pyridin-2-ylsulfonylcyclopropyl)pyrimidin-2-yl]phenyl]-3-(1,2-oxazol-3-yl)urea,
1-[4-[4-[1-(4-chlorophenyl)sulfonylcyclopropyl]-6-[(3S)-3-methylmorpholin-4-yl]pyrimidin-2-yl]phenyl]-3-(2,2-difluoroethyl)urea,
1-[4-[4-[1-(4-chlorophenyl)sulfonylcyclopropyl]-6-[(3S)-3-methylmorpholin-4-yl]pyrimidin-2-yl]phenyl]-3-(1,2-oxazol-3-yl)urea,
3-(2-fluoroethyl)-1-[4-[4-[1-(3-hydroxypropylsulfonyl)cyclopropyl]-6-[(3S)-3-methylmorpholin-4-yl]pyrimidin-2-yl]phenyl]urea,
1-[4-[4-[1-(3-hydroxypropylsulfonyl)cyclopropyl]-6-[(3S)-3-methylmorpholin-4-yl]pyrimidin-2-yl]phenyl]-3-(1,2-oxazol-3-yl)urea,
1-[4-[4-[1-[1-(difluoromethyl)pyrazol-4-yl]sulfonylcyclopropyl]-6-[(3S)-3-methylmorpholin-4-yl]pyrimidin-2-yl]phenyl]-3-(2-hydroxyethyl)urea,
1-[4-[4-[1-(4-fluorophenyl)sulfonylcyclobutyl]-6-[(3S)-3-methylmorpholin-4-yl]pyrimidin-2-yl]phenyl]-3-(1H-imidazol-2-ylmethyl)urea,
1-[4-[4-[1-(4-fluorophenyl)sulfonylcyclobutyl]-6-[(3S)-3-methylmorpholin-4-yl]pyrimidin-2-yl]phenyl]-3-(3-hydroxypropyl)urea,
1-[4-[4-[1-(4-fluorophenyl)sulfonylcyclobutyl]-6-[(3S)-3-methylmorpholin-4-yl]pyrimidin-2-yl]phenyl]-3-(2-hydroxyethyl)urea,
1-[4-[4-[1-(4-fluorophenyl)sulfonylcyclobutyl]-6-[(3S)-3-methylmorpholin-4-yl]pyrimidin-2-yl]phenyl]-3-(1-hydroxy-2-methylpropan-2-yl)urea,
1-[4-[4-[1-(4-fluorophenyl)sulfonylcyclobutyl]-6-[(3S)-3-methylmorpholin-4-yl]pyrimidin-2-yl]phenyl]-3-[(2S)-1-hydroxypropan-2-yl]urea,
1-[4-[4-[1-(4-fluorophenyl)sulfonylcyclobutyl]-6-[(3S)-3-methylmorpholin-4-yl]pyrimidin-2-yl]phenyl]-3-[(2R)-1-hydroxypropan-2-yl]urea,
3-(2-hydroxyethyl)-1-[4-[4-[(3S)-3-methylmorpholin-4-yl]-6-[1-(1,3-thiazol-2-ylsulfonyl)cyclopropyl]pyrimidin-2-yl]phenyl]urea,
3-(3-hydroxypropyl)-1-[4-[4-[(3S)-3-methylmorpholin-4-yl]-6-[1-(1,3-thiazol-2-ylsulfonyl)cyclopropyl]pyrimidin-2-yl]phenyl]urea,
3-(1H-imidazol-2-ylmethyl)-1-[4-[4-[(3S)-3-methylmorpholin-4-yl]-6-[1-(1,3-thiazol-2-ylsulfonyl)cyclopropyl]pyrimidin-2-yl]phenyl]urea,
3-(2-hydroxyethyl)-1-[4-[4-[(3S)-3-methylmorpholin-4-yl]-6-[1-(1,3-thiazol-2-ylsulfonyl)cyclopropyl]pyrimidin-2-yl]phenyl]thiourea,
3-cyclopropyl-1-[4-[4-[(3S)-3-methylmorpholin-4-yl]-6-[1-(4-methylphenyl)sulfonylcyclobutyl]pyrimidin-2-yl]phenyl]urea,
1-[4-[4-[(3S)-3-methylmorpholin-4-yl]-6-[1-(4-methylphenyl)sulfonylcyclobutyl]pyrimidin-2-yl]phenyl]-3-(1-methylpyrazol-4-yl)urea,
1-[4-[4-[1-(benzenesulfonyl)cyclopropyl]-6-[(3R)-3-methylmorpholin-4-yl]pyrimidin-2-yl]phenyl]-3-ethylurea,
1-[4-[4-[1-(benzenesulfonyl)cyclopropyl]-6-[(3S)-3-ethylmorpholin-4-yl]pyrimidin-2-yl]phenyl]-3-ethylurea,
1-[4-[4-[1-(benzenesulfonyl)cyclopropyl]-6-[3-(hydroxymethyl)morpholin-4-yl]pyrimidin-2-yl]phenyl]-3-ethylurea,
1-[4-[4-[1-(benzenesulfonyl)cyclopropyl]-6-[(3S,5S)-3,5-dimethylmorpholin-4-yl]pyrimidin-2-yl]phenyl]-3-ethylurea,
1-[4-[4-[1-(benzenesulfonyl)cyclopropyl]-6-[(3R,5S)-3,5-dimethylmorpholin-4-yl]pyrimidin-2-yl]phenyl]-3-ethylurea,
1-[4-[4-[1-(benzenesulfonyl)cyclopropyl]-6-[(3S)-3-ethylmorpholin-4-yl]pyrimidin-2-yl]phenyl]-3-methylurea,
1-[4-[4-[1-(benzenesulfonyl)cyclopropyl]-6-[(3S)-3-ethylmorpholin-4-yl]pyrimidin-2-yl]phenyl]-3-cyclopropylurea,
1-[4-[4-[1-(benzenesulfonyl)cyclopropyl]-6-[(3S)-3-ethylmorpholin-4-yl]pyrimidin-2-yl]phenyl]-3-(2-hydroxyethyl)urea,
1-[4-[4-[1-(benzenesulfonyl)cyclopropyl]-6-[(3S)-3-ethylmorpholin-4-yl]pyrimidin-2-yl]phenyl]-3-(2-fluoroethyl)urea,
1-[4-[4-[1-(benzenesulfonyl)cyclopropyl]-6-[(3S)-3-ethylmorpholin-4-yl]pyrimidin-2-yl]phenyl]-3-(2,2-difluoroethyl)urea,
1-[4-[4-[1-(benzenesulfonyl)cyclopropyl]-6-[(3S)-3-ethylmorpholin-4-yl]pyrimidin-2-yl]phenyl]-3-(1-methylpyrazol-4-yl)urea,
1-[4-[4-[1-(benzenesulfonyl)cyclopropyl]-6-[(3S)-3-ethylmorpholin-4-yl]pyrimidin-2-yl]phenyl]-3-[(2S)-1-hydroxypropan-2-yl]urea,
1-[4-[4-[1-(benzenesulfonyl)cyclopropyl]-6-[(3S)-3-ethylmorpholin-4-yl]pyrimidin-2-yl]phenyl]-3-[(2R)-1-hydroxypropan-2-yl]urea,
1-[4-[4-[1-(benzenesulfonyl)cyclopropyl]-6-[(3S)-3-ethylmorpholin-4-yl]pyrimidin-2-yl]phenyl]-3-(3-hydroxypropyl)urea,
1-[4-[4-[1-(benzenesulfonyl)cyclopropyl]-6-[(3S)-3-ethylmorpholin-4-yl]pyrimidin-2-yl]phenyl]-3-[(1-hydroxycyclopropyl)methyl]urea,
3-Cyclopropyl-1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]urea,
1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-methylurea,
3-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-1-ethylurea,
1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-(2-hydroxyethyl)urea,
3-(2,2-difluoroethyl)-1-[4-[4-[-[1-(difluoromethyl)-3,5-dimethylpyrazol-4-yl]sulfonylcyclopropyl]-6-[(3S)-3-methylmorpholin-4-yl]pyrimidin-2-yl]phenyl]urea,
1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-(2-fluoroethyl)urea,
1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-(1-methylpyrazol-4-yl)urea,
1-[4-[4-[1-(2-chlorophenyl)sulfonylcyclopropyl]-6-[(3S)-3-methylmorpholin-4-yl]pyrimidin-2-yl]phenyl]-3-cyclopropylurea,
1-[4-[4-[1-(2-chlorophenyl)sulfonylcyclopropyl]-6-[(3S)-3-methylmorpholin-4-yl]pyrimidin-2-yl]phenyl]-3-methylurea,
3-[4-[4-[1-(2-chlorophenyl)sulfonylcyclopropyl]-6-[(3S)-3-methylmorpholin-4-yl]pyrimidin-2-yl]phenyl]-1-ethylurea,
1-[4-[4-[1-(2-chlorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(2-chlorophenyl)sulfonylcyclopropyl]-6-[(3S)-3-methylmorpholin-4-yl]pyrimidin-2-yl]phenyl]-3-(2,2-difluoroethyl)urea,
1-[4-[4-[1-(2-chlorophenyl)sulfonylcyclopropyl]-6-[(3S)-3-methylmorpholin-4-yl]pyrimidin-2-yl]phenyl]-3-(2-fluoroethyl)urea,
1-[4-[4-[1-(2-chlorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-[1-(4-cyanophenyl)sulfonylcyclopropyl]-6-[(3S)-3-methylmorpholin-4-yl]pyrimidin-2-yl]phenyl]-3-cyclopropylurea,
1-[4-[4-[1-(4-cyanophenyl)sulfonylcyclopropyl]-6-[(3S)-3-methylmorpholin-4-yl]pyrimidin-2-yl]phenyl]-3-methylurea,
3-[4-[4-[1-(4-cyanophenyl)sulfonylcyclopropyl]-6-[(3S)-3-methylmorpholin-4-yl]pyrimidin-2-yl]phenyl]-1-ethylurea,
1-[4-[4-[1-(4-cyanophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(4-cyanophenyl)sulfonylcyclopropyl]-6-[(3S)-3-methylmorpholin-4-yl]pyrimidin-2-yl]phenyl]-3-(2,2-difluoroethyl)urea,
1-[4-[4-[1-(4-cyanophenyl)sulfonylcyclopropyl]-6-[(3S)-3-methylmorpholin-4-yl]pyrimidin-2-yl]phenyl]-3-(2-fluoroethyl)urea,
1-[4-[4-[1-(4-cyanophenyl)sulfonylcyclopropyl]-6-[(3S)-3-methylmorpholin-4-yl]pyrimidin-2-yl]phenyl]-3-(1-methylpyrazol-4-yl)urea,
3-cyclopropyl-1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]urea,
1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-methylurea,
3-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-1-ethylurea,
1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-(2-hydroxyethyl)urea,
3-(2,2-difluoroethyl)-1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]urea,
1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-(2-fluoroethyl)urea,
1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-(1-methylpyrazol-4-yl)urea,
3-cyclopropyl-1-[4-[4-[1-(3-hydroxy-3-methylbutyl)sulfonylcyclopropyl]-6-[(3S)-3-methylmorpholin-4-yl]pyrimidin-2-yl]phenyl]urea,
1-[4-[4-[1-(3-hydroxy-3-methylbutyl)sulfonylcyclopropyl]-6-[(3S)-3-methylmorpholin-4-yl]pyrimidin-2-yl]phenyl]-3-methylurea,
3-(2-hydroxyethyl)-1-[4-[4-[1-(3-hydroxy-3-methylbutyl)sulfonylcyclopropyl]-6-[(3S)-3-methylmorpholin-4-yl]pyrimidin-2-yl]phenyl]urea,
3-(2,2-difluoroethyl)-1-[4-[4-[1-(3-hydroxy-3-methylbutyl)sulfonylcyclopropyl]-6-[(3S)-3-methylmorpholin-4-yl]pyrimidin-2-yl]phenyl]urea,
3-(2-fluoroethyl)-1-[4-[4-[1-(3-hydroxy-3-methylbutyl)sulfonylcyclopropyl]-6-[(3S)-3-methylmorpholin-4-yl]pyrimidin-2-yl]phenyl]urea,
1-[4-[4-[1-(3-hydroxy-3-methylbutyl)sulfonylcyclopropyl]-6-[(3S)-3-methylmorpholin-4-yl]pyrimidin-2-yl]phenyl]-3-(1-methylpyrazol-4-yl)urea,
1-ethyl-3-[4-[4-[1-(3-hydroxy-3-methylbutyl)sulfonylcyclopropyl]-6-[(3S)-3-methylmorpholin-4-yl]pyrimidin-2-yl]phenyl]urea,
3-(2,2-difluoroethyl)-1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]urea,
1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-(2-fluoroethyl)urea,
3-cyclopropyl-1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]urea,
1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-(2-hydroxyethyl)urea,
1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-methylurea,
3-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-1-ethylurea,
1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-(1-methylpyrazol-4-yl)urea,
1-[4-[4-[1-(4-fluoro-2-methylphenyl)sulfonylcyclopropyl]-6-[(3S)-3-methylmorpholin-4-yl]pyrimidin-2-yl]phenyl]-3-methylurea,
3-cyclopropyl-1-[4-[4-[1-(4-fluoro-2-methylphenyl)sulfonylcyclopropyl]-6-[(3S)-3-methylmorpholin-4-yl]pyrimidin-2-yl]phenyl]urea,
1-[4-[4-[1-(4-fluoro-2-methylphenyl)sulfonylcyclopropyl]-6-[(3S)-3-methylmorpolin-4-yl]pyrimidin-2-yl]phenyl]-3-(2-hydroxyethyl)urea,
3-(2-fluoroethyl)-1-[4-[4-[1-(4-fluoro-2-methylphenyl)sulfonylcyclopropyl]-6-[(3S)-3-methylmorpholin-4-yl]pyrimidin-2-yl]phenyl]urea,
3-(2,2-difluoroethyl)-1-[4-[4-[1-(4-fluoro-2-methylphenyl)sulfonylcyclopropyl]-6-[(3S)-3-methylmorpholin-4-yl]pyrimidin-2-yl]phenyl]urea,
3-ethyl-1-[4-[4-[1-(4-fluoro-2-methylphenyl)sulfonylcyclopropyl]-6-[(3S)-3-methylmorpholin-4-yl]pyrimidin-2-yl]phenyl]urea,
1-[4-[4-[1-(4-fluoro-2-methylphenyl)sulfonylcyclopropyl]-6-[(3S)-3-methylmorpholin-4-yl]pyrimidin-2-yl]phenyl]-3-(1-methylpyrazol-4-yl)urea,
3-(2-hydroxyethyl)-1-[4-[4-[(3S)-3-methylmorpholin-4-yl]-6-(1-pyridin-4-ylsulfonylcyclobutyl)pyrimidin-2-yl]phenyl]urea,
3-[(2S)-1-hydroxypropan-2-yl]-1-[4-[4-[(3S)-3-methylmorpholin-4-yl]-6-(1-pyridin-4-ylsulfonylcyclobutyl)pyrimidin-2-yl]phenyl]urea,
3-[(2R)-1-hydroxypropan-2-yl]-1-[4-[4-[(3S)-3-methylmorpholin-4-yl]-6-(1-pyridin-4-ylsulfonylcyclobutyl)pyrimidin-2-yl]phenyl]urea,
3-(1-hydroxy-2-methylpropan-2-yl)-1-[4-[4-[(3S)-3-methylmorpholin-4-yl]-6-(1-pyridin-4-ylsulfonylcyclobutyl)pyrimidin-2-yl]phenyl]urea,
3-(1H-imidazol-2-ylmethyl)-1-[4-[4-[(3S)-3-methylmorpholin-4-yl]-6-(1-pyridin-4-ylsulfonylcyclobutyl)pyrimidin-2-yl]phenyl]urea,
1-[4-[4-[(3S)-3-ethylmorpholin-4-yl]-6-(1-methylsulfonylcyclopropyl)pyrimidin-2-yl]phenyl]-3-methylurea,
3-cyclopropyl-1-[4-[4-[(3S)-3-ethylmorpholin-4-yl]-6-(1-methylsulfonylcyclopropyl)pyrimidin-2-yl]phenyl]urea,
1-[4-[4-[(3S)-3-ethylmorpholin-4-yl]-6-(1-methylsulfonylcyclopropyl)pyrimidin-2-yl]phenyl]-3-(2-hydroxyethyl)urea,
1-[4-[4-[(3S)-3-ethylmorpholin-4-yl]-6-(1-methylsulfonylcyclopropyl)pyrimidin-2-yl]phenyl]-3-(2-fluoroethyl)urea,
3-(2,2-difluoroethyl)-1-[4-[4-[(3S)-3-ethylmorpholin-4-yl]-6-(1-methylsulfonylcyclopropyl)pyrimidin-2-yl]phenyl]urea,
3-ethyl-1-[4-[4-[(3S)-3-ethylmorpholin-4-yl]-6-(1-methylsulfonylcyclopropyl)pyrimidin-2-yl]phenyl]urea,
1-[4-[4-[(3S)-3-ethylmorpholin-4-yl]-6-(1-methylsulfonylcyclopropyl)pyrimidin-2yl]phenyl]-3-(1-methylpyrazol-4-yl)urea
3-chloro-4-[1-[2-[4-(methylcarbamoylamino)phenyl]-6-[(3S)-3-methylmorpholin-4-yl]pyrimidin-4-yl]cyclopropyl]sulfonylbenzamide
3-chloro-4-[1-[2-[4-(ethylcarbamoylamino)phenyl]-6-[(3S)-3-methylmorpholin-4-yl]pyrimidin-4-yl]cyclopropyl]sulfonylbenzamide
3-chloro-4-[1-[2-[4-(cyclopropylcarbamoylamino)phenyl]-6-[(3S)-3-methylmorpholin-4-yl]pyrimidin-4-yl]cyclopropyl]sulfonylbenzamide
3-chloro-4-[1-[2-[4-(2-fluoroethylcarbamoylamino)phenyl]-6-[(3S)-3-methylmorpholin-4-yl]pyrimidin-4-yl]cyclopropyl]sulfonylbenzamide
3-chloro-4-[1-[2-[4-(2-hydroxyethylcarbamoylamino)phenyl]-6-[(3S)-3-methylmorpholin-4-yl]pyrimidin-4-yl]cyclopropyl]sulfonylbenzamide
1-[4-[4-[1-(2-chloro-4-cyanophenyl)sulfonylcyclopropyl]-6-[(3S)-3-methylmorpholin-4-yl]pyrimidin-2-yl]phenyl]-3-methylurea
3-[4-[4-[1-(2-chloro-4-cyanophenyl)sulfonylcyclopropyl]-6-[(3S)-3-methylmorpholin-4-yl]pyrimidin-2-yl]phenyl]-1-ethylurea
1-[4-[4-[1-(2-chloro-4-cyanophenyl)sulfonylcyclopropyl]-6-[(3S)-3-methylmorpholin-4-yl]pyrimidin-2-yl]phenyl]-3-cyclopropylurea
1-[4-[4-[1-(2-chloro-4-cyanophenyl)sulfonylcyclopropyl]-6-[(3S)-3-methylmorpholin-4-yl]pyrimidin-2-yl]phenyl]-3-(2,2-difluoroethyl)urea
1-[4-[4-[1-(2-chloro-4-cyanophenyl)sulfonylcyclopropyl]-6-[(3S)-3-methylmorpholin-4-yl]pyrimidin-2-yl]phenyl]-3-(2-fluoroethyl)urea
1-[4-[4-[1-(2-chloro-4-cyanophenyl)sulfonylcyclopropyl]-6-[(3S)-3-methylmorpholin-4-yl]pyrimidin-2-yl]phenyl]-3-(2-hydroxyethyl)urea
1-[4-[4-[1-(2,6-difluorophenyl)sulfonylcyclopropyl]-6-[(3S)-3-methylmorpholin-4-yl]pyrimidin-2-yl]phenyl]-3-methylurea
1-[4-[4-[1-(2,6-difluorophenyl)sulfonylcyclopropyl]-6-[(3S)-3-methylmorpholin-4-yl]pyrimidin-2-yl]phenyl]-3-ethylurea
3-cyclopropyl-1-[4-[4-[1-(2,6-difluorophenyl)sulfonylcyclopropyl]-6-[(3S)-3-methylmorpholin-4-yl]pyrimidin-2-yl]phenyl]urea
1-[4-[4-[1-(2,6-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(2,6-difluorophenyl)sulfonylcyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-(2,6-difluorophenyl)sulfonylcyclopropyl]-6-[(3S)-3-methylmorpholin-4-yl]pyrimidin-2-yl]phenyl]urea,
1-[4-[4-[1-(2,6-difluorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-(1-ethylsulfonylcyclobutyl)-6-[(3S)-3-methylmorpholin-4-yl]pyrimidin-2-yl]phenyl]-3-(2-hydroxyethyl)urea,
1-[4-[4-(1-ethylsulfonylcyclobutyl)-6-[(3S)-3-methylmorpholin-4-yl]pyrimidin-2-yl]phenyl]-3-(1H-imidazol-2-ylmethyl)urea,
1-[4-[4-(1-ethylsulfonylcyclobutyl)-6-[(3S)-3-methylmorpholin-4-yl]pyrimidin-2-yl]phenyl]-3-(3-hydroxypropyl)urea,
1-[4-[4-(1-ethylsulfonylcyclobutyl)-6-[(3S)-3-methylmorpholin-4-yl]pyrimidin-2-yl]phenyl]-3-methylurea,
1-[4-[4-(1-ethylsulfonylcyclobutyl)-6-[(3S)-3-methylmorpholin-4-yl]pyrimidin-2-yl]phenyl]-3-(1-hydroxy-2-methylpropan-2-yl)urea,
1-[4-[4-(1-ethylsulfonylcyclobutyl)-6-[(3S)-3-methylmorpholin-4-yl]pyrimidin-2-yl]phenyl]-3-[(2S)-1-hydroxypropan-2-yl]urea,
1-[4-[4-(1-ethylsulfonylcyclobutyl)-6-[(3S)-3-methylmorpholin-4-yl]pyrimidin-2-yl]phenyl]-3-[(2R)-1-hydroxypropan-2-yl]urea,
1-[4-[4-(1-ethylsulfonylcyclobutyl)-6-[(3S)-3-methylmorpholin-4-yl]pyrimidin-2-yl]phenyl]-3-(3-hydroxypropyl)thiourea,
1-[4-[4-(1-ethylsulfonylcyclobutyl)-6-[(3S)-3-methylmorpholin-4-yl]pyrimidin-2-yl]phenyl]-3-(2-hydroxyethyl)thiourea,
1-[4-[4-(1-ethylsulfonylcyclobutyl)-6-[(3S)-3-methylmorpholin-4-yl]pyrimidin-2-yl]phenyl]-3-(1H-imidazol-2-ylmethyl)thiourea,
1-[4-[4-(1-ethylsulfonylcyclopropyl)-6-[(3S)-3-methylmorpholin-4-yl]pyrimidin-2-yl]phenyl]-3-(3-hydroxypropyl)thiourea,
1-[4-[4-(1-ethylsulfonylcyclopropyl)-6-[(3S)-3-methylmorpholin-4-yl]pyrimidin-2-yl]phenyl]-3-(2-hydroxyethyl)thiourea,
1-[4-[4-(1-ethylsulfonylcyclopropyl)-6-[(3S)-3-methylmorpholin-4-yl]pyrimidin-2-yl]phenyl]-3-(1H-imidazol-2-ylmethyl)thiourea,
4-[6-[1-(benzenesulfonyl)cyclopropyl]-2-[4-(ethylcarbamoylamino)phenyl]pyrimidin-4-yl]morpholine-3-carboxamide,
4-[6-[1-(benzenesulfonyl)cyclopropyl]-2-[4-(ethylcarbamoylamino)phenyl]pyrimidin-4-yl]-N,N-dimethylmorpholine-3-carboxamide,
3-cyclopropyl-1-[4-[4-[(3S,5S)-3,5-dimethylmorpholin-4-yl]-6-(1-methylsulfonylcyclopropyl)pyrimidin-2-yl]phenyl]urea,
1-[4-[4-[(3S,5S)-3,5-dimethylmorpholin-4-yl]-6-(1-methylsulfonylcyclopropyl)pyrimidin-2-yl]phenyl]-3-methylurea,
1-[4-[4-[(3S,5S)-3,5-dimethylmorpholin-4-yl]-6-(1-methylsulfonylcyclopropyl)pyrimidin-2-yl]phenyl]-3-ethylurea,
1-[4-[4-[(3S,5S)-3,5-dimethylmorpholin-4-yl]-6-(1-methylsulfonylcyclopropyl)pyrimidin-2-yl]phenyl]-3-(2-fluoroethyl)urea,
1-[4-[4-[(3S,5S)-3,5-dimethylmorpholin-4-yl]-6-(1-methylsulfonylcyclopropyl)pyrimidin-2-yl]phenyl]-3-(2-hydroxyethyl)urea,
3-cyclopropyl-1-[4-[4-(1-cyclopropylsulfonylcyclopropyl)-6-[(3S,5S)-3,5-dimethylmorpholin-4-yl]pyrimidin-2-yl]phenyl]urea,
1-[4-[4-(1-cyclopropylsulfonylcyclopropyl)-6-[(3S,5S)-3,5-dimethylmorpholin-4-yl]pyrimidin-2-yl]phenyl]-3-(2,2-difluoroethyl)urea,
1-[4-[4-(1-cyclopropylsulfonylcyclopropyl)-6-[(3S,5S)-3,5-dimethylmorpholin-4-yl]pyrimidin-2-yl]phenyl]-3-(1-methylpyrazol-4-yl)urea,
1-[4-[4-(1-cyclopropylsulfonylcyclopropyl)-6-[(3S,5S)-3,5-dimethylmorpholin-4-yl]pyrimidin-2-yl]phenyl]-3-methylurea,
1-[4-[4-(1-cyclopropylsulfonylcyclopropyl)-6-[(3S,5S)-3,5-dimethylmorpholin-4-yl]pyrimidin-2-yl]phenyl]-3-ethylurea,
1-[4-[4-(1-cyclopropylsulfonylcyclopropyl)-6-[(3S,5S)-3,5-dimethylmorpholin-4-yl]pyrimidin-2-yl]phenyl]-3-(2-fluoroethyl)urea,
1-[4-[4-(1-cyclopropylsulfonylcyclopropyl)-6-[(3S,5S)-3,5-dimethylmorpholin-4-yl]pyrimidin-2-yl]phenyl]-3-(2-hydroxyethyl)urea,
1-[4-[4-[1-(benzenesulfonyl)cyclopropyl]-6-[(3S,5S)-3,5-dimethylmorpholin-4-yl]pyrimidin-2-yl]phenyl]-3-cyclopropylurea,
1-[4-[4-[1-(benzenesulfonyl)cyclopropyl]-6-[(3S,5S)-3,5-dimethylmorpholin-4-yl]pyrimidin-2-yl]phenyl]-3-methylurea
1-[4-[4-[1-(benzenesulfonyl)cyclopropyl]-6-[(3S,5S)-3,5-dimethylmorpholin-4-yl]pyrimidin-2-yl]phenyl]-3-(2-hydroxyethyl)urea
1-[4-[4-[1-(benzenesulfonyl)cyclopropyl]-6-[(3S,5S)-3,5-dimethylmorpholin-4-yl]pyrimidin-2-yl]phenyl]-3-(2-fluoroethyl)urea
1-[4-[4-[1-(benzenesulfonyl)cyclopropyl]-6-[(3S,5S)-3,5-dimethylmorpholin-4-yl]pyrimidin-2-yl]phenyl]-3-(2,2-difluoroethyl)urea
1-[4-[4-[1-(benzenesulfonyl)cyclopropyl]-6-[(3S,5S)-3,5-dimethylmorpholin-4-yl]pyrimidin-2-yl]phenyl]-3-(1-methylpyrazol-4-yl)urea
3-methyl-1-[4-[4-[(3S)-3-methylmorpholin-4-yl]-6-[1-(oxan-4-ylsulfonyl)cyclopropyl]pyrimidin-2-yl]phenyl]urea
1-ethyl-3-[4-[4-[(3S)-3-methylmorpholin-4-yl]-6-[1-(oxan-4-ylsulfonyl)cyclopropyl]pyrimidin-2-yl]phenyl]urea
3-cyclopropyl-1-[4-[4-[(3S)-3-methylmorpholin-4-yl]-6-[1-(oxan-4-ylsulfonyl)cyclopropyl]pyrimidin-2-yl]phenyl]urea
3-(2-hydroxyethyl)-1-[4-[4-[(3S)-3-methylmorpholin-4-yl]-6-[1-(oxan-4-ylsulfonyl)cyclopropyl]pyrimidin-2-yl]phenyl]urea
1-[4-[4-[(3S)-3-methylmorpholin-4-yl]-6-[1-(oxan-4-ylsulfonyl)cyclopropyl]pyrimidin-2-yl]phenyl]-3-(1-methylpyrazol-4-yl)urea
3-cyclobutyl-1-[4-[4-[(3S)-3-methylmorpholin-4-yl]-6-[1-(oxan-4-ylsulfonyl)cyclopropyl]pyrimidin-2-yl]phenyl]urea
3-methyl-1-[4-[4-[(3S)-3-methylmorpholin-4-yl]-6-[1-(oxolan-3-ylsulfonyl)cyclopropyl]pyrimidin-2-yl]phenyl]urea
1-ethyl-3-[4-[4-[(3S)-3-methylmorpholin-4-yl]-6-[1-(oxolan-3-ylsulfonyl)cyclopropyl]pyrimidin-2-yl]phenyl]urea
3-cyclopropyl-1-[4-[4-[(3S)-3-methylmorpholin-4-yl]-6-[1-(oxolan-3-ylsulfonyl)cyclopropyl]pyrimidin-2-yl]phenyl]urea
3-(2-hydroxyethyl)-1-[4-[4-[(3S)-3-methylmorpholin-4-yl]-6-[1-(oxolan-3-ylsulfonyl)cyclopropyl]pyrimidin-2-yl]phenyl]urea
1-[4-[4-[(3S)-3-methylmorpholin-4-yl]-6-[1-(oxolan-3-ylsulfonyl)cyclopropyl]pyrimidin-2-yl]phenyl]-3-(1-methylpyrazol-4-yl)urea,
1-[4-[4-[1-(3-chloro-4-fluorophenyl)sulfonylcyclopropyl]-6-[(3S)-3-methylmorpholin-4-yl]pyrimidin-2-yl]phenyl]-3-methylurea,
1-[4-[4-[1-(3-chloro-4-fluorophenyl)sulfonylcyclopropyl]-6-[(3S)-3-methylmorpholin-4-yl]pyrimidin-2-yl]phenyl]-3-cyclopropylurea,
1-[4-[4-[1-(3-chloro-4-fluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(3-chloro-4-fluorophenyl)sulfonylcyclopropyl]-6-[(3S)-3-methylmorpholin-4-yl]pyrimidin-2-yl]phenyl]-3-(2-fluoroethyl)urea,
1-[4-[4-[1-(3-chloro-4-fluorophenyl)sulfonylcyclopropyl]-6-[(3S)-3-methylmorpholin-4-yl]pyrimidin-2-yl]phenyl]-3-(2,2-difluoroethyl)urea,
3-[4-[4-[1-(3-chloro-4-fluorophenyl)sulfonylcyclopropyl]-6-[(3S)-3-methylmorpholin-4-yl]pyrimidin-2-yl]phenyl]-1-ethylurea,
1-[4-[4-[1-(3-chloro-4-fluorophenyl)sulfonylcyclopropyl]-6-[(3S)-3-methylmorpholin-4-yl]pyrimidin-2-yl]phenyl]-3-(1-methylpyrazol-4-yl)urea,
1-[4-[4-[1-(3-chloro-4-methylaminophenyl)sulfonylcyclopropyl]-6-[(3S)-3-methylmorpholin-4-yl]pyrimidin-2-yl]phenyl]-3-methylurea,
1-[4-[4-[1-[3-chloro-4-(2-hydroxyethylamino)phenyl]sulfonylcyclopropyl]-6-[(3S)-3-methylmorpholin-4-yl]pyrimidin-2-yl]phenyl]-3-(2-hydroxyethyl)urea,
1-[4-[4-[1-[3-chloro-4-(2-fluoroethylamino)phenyl]sulfonylcyclopropyl]-6-[(3S)-3-methylmorpholin-4-yl]pyrimidin-2-yl]phenyl]-3-(2-fluoroethyl)urea,
1-[4-[4-[1-(3-chloro-4-ethylaminophenyl)sulfonylcyclopropyl]-6-[(3S)-3-methylmorpholin-4-yl]pyrimidin-2-yl]phenyl]-3-ethylurea,
3-methyl-1-[4-[4-[(3S)-3-methylmorpholin-4-yl]-6-[1-(4-methylphenyl)sulfonylcyclopropyl]pyrimidin-2-yl]phenyl]urea,
3-cyclopropyl-1-[4-[4-[(3S)-3-methylmorpholin-4-yl]-6-[1-(4-methylphenyl)sulfonylcyclopropyl]pyrimidin-2-yl]phenyl]urea,
3-(2-hydroxyethyl)-1-[4-[4-[(3S)-3-methylmorpholin-4-yl]-6-[1-(4-methylphenyl)sulfonylcyclopropyl]pyrimidin-2-yl]phenyl]urea,
3-(2-fluoroethyl)-1-[4-[4-[(3S)-3-methylmorpholin-4-yl]-6-[1-(4-methylphenyl)sulfonylcyclopropyl]pyrimidin-2-yl]phenyl]urea,
3-(2,2-difluoroethyl)-1-[4-[4-[(3S)-3-methylmorpholin-4-yl]-6-[1-(4-methylphenyl)sulfonylcyclopropyl]pyrimidin-2-yl]phenyl]urea,
1-ethyl-3-[4-[4-[(3S)-3-methylmorpholin-4-yl]-6-[1-(4-methylphenyl)sulfonylcyclopropyl]pyrimidin-2-yl]phenyl]urea,
1-[4-[4-[(3S)-3-methylmorpholin-4-yl]-6-[1-(4-methylphenyl)sulfonylcyclopropyl]pyrimidin-2-yl]phenyl]-3-(1-methylpyrazol-4-yl)urea,
1-[4-[4-[1-[3-(difluoromethoxy)propylsulfonyl]cyclopropyl]-6-[(3S)-3-methylmorpholin-4-yl]pyrimidin-2-yl]phenyl]-3-(2-fluoroethyl)urea,
3-(2,2-difluoroethyl)-1-[4-[4-[1-[3-(difluoromethoxy)propylsulfonyl]cyclopropyl]-6-[(3S)-3-methylmorpholin-4-yl]pyrimidin-2-yl]phenyl]urea,
1-[4-[4-[1-[3-(difluoromethoxy)propylsulfonyl]cyclopropyl]-6-[(3S)-3-methylmorpholin-4-yl]pyrimidin-2-yl]phenyl]-3-ethylurea,
1-[4-[4-[1-[3-(difluoromethoxy)propylsulfonyl]cyclopropyl]-6-[(3S)-3-methylmorpholin-4-yl]pyrimidin-2-yl]phenyl]-3-(1-methylpyrazol-4-yl)urea,
3-cyclopropyl-1-[4-[4-[1-[3-(difluoromethoxy)propylsulfonyl]cyclopropyl]-6-[(3S)-3-methylmorpholin-4-yl]pyrimidin-2-yl]phenyl]urea,
3-methyl-1-[4-[4-[1-(2-methylaminoethylsulfonyl)cyclopropyl]-6-morpholin-4-ylpyrimidin-2-yl]phenyl]urea,
3-(1-hydroxy-2-methylpropan-2-yl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-[(2S)-1-hydroxypropan-2-yl]-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-(1H-imidazol-2-ylmethyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-(3-hydroxypropyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-(2-hydroxyethyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-[(2R)-1-hydroxypropan-2-yl]-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-(1-hydroxy-2-methylpropan-2-yl)-1-[4-[4-[(3S)-3-methylmorpholin-4-yl]-6-[1-(1,3-thiazol-2-ylsulfonyl)cyclobutyl]pyrimidin-2-yl]phenyl]urea,
3-[(2S)-1-hydroxypropan-2-yl]-1-[4-[4-[(3S)-3-methylmorpholin-4-yl]-6-[1-(1,3-thiazol-2-ylsulfonyl)cyclobutyl]pyrimidin-2-yl]phenyl]urea,
3-(1H-imidazol-2-ylmethyl)-1-[4-[4-[(3S)-3-methylmorpholin-4-yl]-6-[1-(1,3-thiazol-2-ylsulfonyl)cyclobutyl]pyrimidin-2-yl]phenyl]urea,
3-(3-hydroxypropyl)-1-[4-[4-[(3S)-3-methylmorpholin-4-yl]-6-[1-(1,3-thiazol-2-ylsulfonyl)cyclobutyl]pyrimidin-2-yl]phenyl]urea,
3-(2-hydroxyethyl)-1-[4-[4-[(3S)-3-methylmorpholin-4-yl]-6-[1-(1,3-thiazol-2-ylsulfonyl)cyclobutyl]pyrimidin-2-yl]phenyl]urea,
3-[(2R)-1-hydroxypropan-2-yl]-1-[4-[4-[(3S)-3-methylmorpholin-4-yl]-6-[1-(1,3-thiazol-2-ylsulfonyl)cyclobutyl]pyrimidin-2-yl]phenyl]urea,
1-[4-[4-[1-(3-hydroxypropylsulfonyl)cyclobutyl]-6-[(3S)-3-methylmorpholin-4-yl]pyrimidin-2-yl]phenyl]-3-methylthiourea,
3-cyclopropyl-1-[4-[4-[1-(3-hydroxypropylsulfonyl)cyclobutyl]-6-[(3S)-3-methylmorpholin-4-yl]pyrimidin-2-yl]phenyl]thiourea,
1-[4-[4-[1-(3-hydroxypropylsulfonyl)cyclobutyl]-6-[(3S)-3-methylmorpholin-4-yl]pyrimidin-2-yl]phenyl]-3-(1H-imidazol-2-ylmethyl)thiourea,
3-(1-hydroxy-2-methylpropan-2-yl)-1-[4-[4-[1-(3-hydroxypropylsulfonyl)cyclobutyl]-6-[(3S)-3-methylmorpholin-4-yl]pyrimidin-2-yl]phenyl]thiourea,
1-ethyl-3-[4-[4-[1-(3-hydroxypropylsulfonyl)cyclobutyl]-6-[(3S)-3-methylmorpholin-4-yl]pyrimidin-2-yl]phenyl]thiourea,
3-(2-hydroxyethyl)-1-[4-[4-[1-(3-hydroxypropylsulfonyl)cyclobutyl]-6-[(3S)-3-methylmorpholin-4-yl]pyrimidin-2-yl]phenyl]thiourea,
3-(2,2-difluoroethyl)-1-[4-[4-[1-(3-hydroxypropylsulfonyl)cyclobutyl]-6-[(3S)-3-methylmorpholin-4-yl]pyrimidin-2-yl]phenyl]thiourea,
3-[4-[4-[1-(3-hydroxypropylsulfonyl)cyclobutyl]-6-[(3S)-3-methylmorpholin-4-yl]pyrimidin-2-yl]phenyl]-1-propylthiourea,
3-(3-hydroxypropyl)-1-[4-[4-[1-(3-hydroxypropylsulfonyl)cyclobutyl]-6-[(3S)-3-methylmorpholin-4-yl]pyrimidin-2-yl]phenyl]thiourea, and
3-(2,2-difluoroethyl)-1-[4-[4-[1-(3-hydroxypropylsulfonyl)cyclopropyl]-6-[(3S)-3-methylmorpholin-4-yl]pyrimidin-2-yl]phenyl]thiourea,
   or a pharmaceutically acceptable salt thereof.

In another aspect of the invention there is provided a compound, or a combination of compounds, selected from any one of
1-[4-[4-(1-ethylsulfonylcyclopropyl)-6-[(3S)-3-methylmorpholin-4-yl]pyrimidin-2-yl]phenyl]-3-methyl-urea,
1-[4-[4-(1-cyclopropylsulfonylcyclopropyl)-6-[(3S)-3-methylmorpholin-4-yl]pyrimidin-2-yl]phenyl]-3-(2-hydroxyethyl)urea,
3-(2-hydroxyethyl)-1-[4-[4-[(3S)-3-methylmorpholin-4-yl]-6-(1-methylsulfonylcyclopropyl)pyrimidin-2-yl]phenyl]thiourea,
3-cyclopropyl-1-[4-[4-(1-methylsulfonylcyclopropyl)-6-morpholin-4-ylpyrimidin-2-yl]phenyl]urea,
1-[4-[4-[1-(4-chlorophenyl)sulfonylcyclopropyl]-6-[(3S)-3-methylmorpholin-4-yl]pyrimidin-2-yl]phenyl]-3-(2-hydroxyethyl)urea,
3-(2-hydroxyethyl)-1-[4-[4-[(3S)-3-methylmorpholin-4-yl]-6-(1-pyridin-2-ylsulfonylcyclopropyl)pyrimidin-2-yl]phenyl]urea,
3-(2-hydroxyethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
1-[4-[4-[1-(benzenesulfonyl)cyclopropyl]-6-[(3S)-3-methylmorpholin-4-yl]pyrimidin-2-yl]phenyl]-3-cyclopropylurea,
1-[4-[4-[1-(benzenesulfonyl)cyclopropyl]-6-[(3S)-3-methylmorpholin-4-yl]pyrimidin-2-yl]phenyl]-3-(2-hydroxyethyl)urea,
1-[4-[4-[1-(benzenesulfonyl)cyclopropyl]-6-[(3S)-3-methylmorpholin-4-yl]pyrimidin-2-yl]phenyl]-3-(3-hydroxypropyl)urea,
1-[4-[4-(1-cyclopropylsulfonylcyclopropyl)-6-[(3S)-3-methylmorpholin-4-yl]pyrimidin-2-yl]phenyl]-3-(2-hydroxyethyl)thiourea,
3-cyclopropyl-1-[4-[4-[1-(4-fluorophenyl)sulfonylcyclopropyl]-6-[(3S)-3-methylmorpholin-4-yl]pyrimidin-2-yl]phenyl]thiourea,
3-cyclopropyl-1-[4-[4-[1-(3-hydroxypropylsulfonyl)cyclopropyl]-6-[(3S)-3-methylmorpholin-4-yl]pyrimidin-2-yl]phenyl]thiourea,
1-ethyl-3-[4-[4-[1-(3-hydroxypropylsulfonyl)cyclopropyl]-6-[(3S)-3-methylmorpholin-4-yl]pyrimidin-2-yl]phenyl]thiourea,
1-[4-[4-(1-cyclopropylsulfonylcyclobutyl)-6-[(3S)-3-methylmorpholin-4-yl]pyrimidin-2-yl]phenyl]-3-(2-hydroxyethyl)urea,
1-[4-[4-[1-(benzenesulfonyl)cyclobutyl]-6-[(3S)-3-methylmorpholin-4-yl]pyrimidin-2-yl]phenyl]-3-(2-hydroxyethyl)urea,
1-[4-[4-[1-(benzenesulfonyl)cyclopropyl]-6-[(3S)-3-methylmorpholin-4-yl]pyrimidin-2-yl]phenyl]-3-methylthiourea,
1-[4-[4-[1-(benzenesulfonyl)cyclopropyl]-6-[(3S)-3-methylmorpholin-4-yl]pyrimidin-2-yl]phenyl]-3-(1H-imidazol-2-ylmethyl)thiourea,
3-(2-hydroxyethyl)-1-[4-[4-[(3S)-3-methylmorpholin-4-yl]-6-(1-pyridin-4-ylsulfonylcyclopropyl)pyrimidin-2-yl]phenyl]thiourea,
1-[4-[4-[1-(benzenesulfonyl)cyclopropyl]-6-[(3S)-3-methylmorpholin-4-yl]pyrimidin-2-yl]phenyl]-3-(2-fluoroethyl)urea,
1-[4-[4-[1-(2,4-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(3-fluorophenyl)sulfonylcyclopropyl]-6-[(3S)-3-methylmorpholin-4-yl]pyrimidin-2-yl]phenyl]-3-(2-hydroxyethyl)urea,
3-(2-hydroxyethyl)-1-[4-[4-[(3S)-3-methylmorpholin-4-yl]-6-[1-(2-methylphenyl)sulfonylcyclopropyl]pyrimidin-2-yl]phenyl]urea,
3-(2-fluoroethyl)-1-[4-[4-[(3S)-3-methylmorpholin-4-yl]-6-[1-(2-methylphenyl)sulfonylcyclopropyl]pyrimidin-2-yl]phenyl]urea,
3-(2-hydroxyethyl)-1-[4-[4-[(3S)-3-methylmorpholin-4-yl]-6-[1-(1,3-thiazol-2-ylsulfonyl)cyclopropyl]pyrimidin-2-yl]phenyl]urea,
1-[4-[4-[1-(2-chlorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(benzenesulfonyl)cyclobutyl]-6-[(3S)-3-methylmorpholin-4-yl]pyrimidin-2-yl]phenyl]-3-[(2S)-1-hydroxypropan-2-yl]urea,
1-[4-[4-(1-cyclopropylsulfonylcyclobutyl)-6-[(3S)-3-methylmorpholin-4-yl]pyrimidin-2-yl]phenyl]-3-[(2S)-1-hydroxypropan-2-yl]urea,
1-[4-[4-[1-(benzenesulfonyl)cyclobutyl]-6-[(3S)-3-methylmorpholin-4-yl]pyrimidin-2-yl]phenyl]-3-[(2R)-1-hydroxypropan-2-yl]urea,
3-(2-fluoroethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-hydroxyethyl)-1-[4-[4-[(3S)-3-methylmorpholin-4-yl]-6-[1-[4-methyl-1,3-thiazol-2-yl)sulfonyl]cyclobutyl]pyrimidin-2-yl]phenyl]urea,
3-(2-hydroxyethyl)-1-[4-[4-[(3S)-3-methylmorpholin-4-yl]-6-[1-(1,3-thiazol-2-ylsulfonyl)cyclobutyl]pyrimidin-2-yl]phenyl]urea,
1-[4-[4-[1-(benzenesulfonyl)cyclopropyl]-6-[(3S)-3-ethylmorpholin-4-yl]pyrimidin-2-yl]phenyl]-3-methylurea,
1-[4-[4-[1-(benzenesulfonyl)cyclopropyl]-6-[(3S)-3-ethylmorpholin-4-yl]pyrimidin-2-yl]phenyl]-3-cyclopropylurea,
1-[4-[4-[1-(benzenesulfonyl)cyclopropyl]-6-[(3S)-3-ethylmorpholin-4-yl]pyrimidin-2-yl]phenyl]-3-(2-hydroxyethyl)urea,
1-[4-[4-[1-(benzenesulfonyl)cyclopropyl]-6-[(3S)-3-ethylmorpholin-4-yl]pyrimidin-2-yl]phenyl]-3-(2-fluoroethyl)urea,
1-[4-[4-[1-(benzenesulfonyl)cyclopropyl]-6-[(3S)-3-methylmorpholin-4-yl]pyrimidin-2-yl]phenyl]-3-(2,2-difluoroethyl)urea,
1-[4-[4-[1-(benzenesulfonyl)cyclopropyl]-6-[(3S)-3-methylmorpholin-4-yl]pyrimidin-2-yl]phenyl]-3-[(2S)-1-hydroxypropan-2-yl]urea,
1-[4-[4-[1-(benzenesulfonyl)cyclopropyl]-6-[(3S)-3-methylmorpholin-4-yl]pyrimidin-2-yl]phenyl]-3-[(2R)-1-hydroxypropan-2-yl]urea,
3-cyclopropyl-1-[4-[4-[(3S)-3-ethylmorpholin-4-yl]-6-(1-methylsulfonylcyclopropyl)pyrimidin-2-yl]phenyl]urea,
1-[4-[4-[(3S)-3-ethylmorpholin-4-yl]-6-(1-methylsulfonylcyclopropyl)pyrimidin-2-yl]phenyl]-3-(2-hydroxyethyl)urea,
1-[4-[4-[1-(2,6-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-(1-cyclopropylsulfonylcyclopropyl)-6-[(3S,5S)-3,5-dimethylmorpholin-4-yl]pyrimidin-2-yl]phenyl]-3-(2-hydroxyethyl)urea,
3-cyclopropyl-1-[4-[4-[(3S,5S)-3,5-dimethylmorpholin-4-yl]-6-(1-methylsulfonylcyclopropyl)pyrimidin-2-yl]phenyl]urea,
1-[4-[4-[(3S,5S)-3,5-dimethylmorpholin-4-yl]-6-(1-methylsulfonylcyclopropyl)pyrimidin-2-yl]phenyl]-3-(2-fluoroethyl)urea,
1-[4-[4-[(3S,5S)-3,5-dimethylmorpholin-4-yl]-6-(1-methylsulfonylcyclopropyl)pyrimidin-2-yl]phenyl]-3-(2-hydroxyethyl)urea,
1-[4-[4-[1-(benzenesulfonyl)cyclopropyl]-6-[(3S,5S)-3,5-dimethylmorpholin-4-yl]pyrimidin-2-yl]phenyl]-3-methylurea,
1-[4-[4-[1-(benzenesulfonyl)cyclopropyl]-6-[(3S,5S)-3,5-dimethylmorpholin-4-yl]pyrimidin-2-yl]phenyl]-3-cyclopropylurea,
1-[4-[4-[1-(benzenesulfonyl)cyclopropyl]-6-[(3S,5S)-3,5-dimethylmorpholin-4-yl]pyrimidin-2-yl]phenyl]-3-(2-hydroxyethyl)urea, and
1-[4-[4-[1-(benzenesulfonyl)cyclopropyl]-6-[(3S)-3-ethylmorpholin-4-yl]pyrimidin-2-yl]phenyl]-3-[(2S)-1-hydroxypropan-2-yl]urea,
1-[4-[4-[1-(benzenesulfonyl)cyclopropyl]-6-[(3S)-3-ethylmorpholin-4-yl]pyrimidin-2-yl]phenyl]-3-(3-hydroxypropyl)urea,
1-[4-[4-[1-(benzenesulfonyl)cyclopropyl]-6-[(3S)-3-ethylmorpholin-4-yl]pyrimidin-2-yl]phenyl]-3-[(1-hydroxycyclopropyl)methyl]urea,
1-[4-[4-(1-ethylsulfonylcyclobutyl)-6-[(3S)-3-methylmorpholin-4-yl]pyrimidin-2-yl]phenyl]-3-(2-hydroxyethyl)thiourea,
1-[4-[4-[(3S,5S)-3,5-dimethylmorpholin-4-yl]-6-(1-methylsulfonylcyclopropyl)pyrimidin-2-yl]phenyl]-3-ethylurea,
3-[(2S)-1-hydroxypropan-2-yl]-1-[4-[4-[(3S)-3-methylmorpholin-4-yl]-6-[1-(1,3-thiazol-2-ylsulfonyl)cyclobutyl]pyrimidin-2-yl]phenyl]urea, and
1-[4-[4-[1-(benzenesulfonyl)cyclopropyl]-6-[(3S,5S)-3,5-dimethylmorpholin-4-yl]pyrimidin-2-yl]phenyl]-3-(2-fluoroethyl)urea,
   or a pharmaceutically acceptable salt thereof.

The invention also provides processes for the preparation of a compound of formula (I) or a pharmaceutically acceptable salt thereof.

A compound of formula (I), wherein X═—S(O)₂CR⁶R⁷—, may be prepared by oxidising a compound of the formula (I), wherein X═SCR⁶R⁷—, for example by using Oxone® at room temperature in a mixed solvent system of water and ethanol

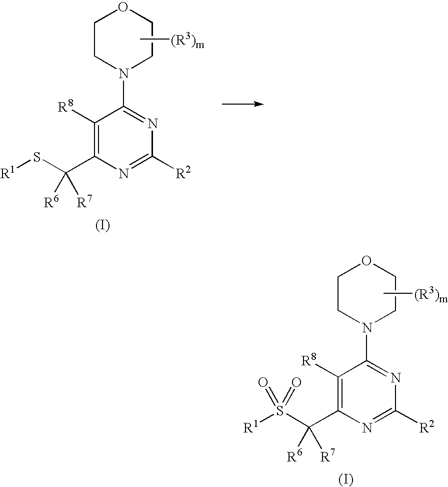

A compound of formula (I), wherein R¹X═R¹OCR⁶R⁷—, may be prepared by the reaction of a compound of formula (I), wherein R¹X═HOCR⁶R⁷—, with a compound of formula (II), wherein L¹ is a leaving group (such as halo, tosyl, mesyl etc.) optionally in the presence of a suitable base such as triethylamine and a solvent such as tetrahydrofuran or N,N-dimethylformamide.

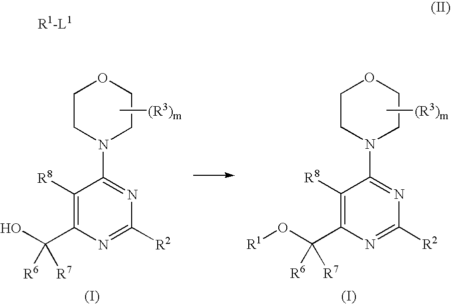

A compound of formula (I), wherein R¹X═R¹R⁴NCR⁶R⁷—, may be prepared by the reaction of a compound of formula (I), wherein R¹X═HR⁴NCR⁶R⁷—, with a compound of formula (II), wherein L¹ is a leaving group (such as halo, tosyl, mesyl etc.) optionally in the presence of a suitable base such as triethylamine and a solvent such as tetrahydrofuran or N,N-dimethylformamide; or by the reaction of a compound of formula (I), wherein R¹X═HR⁴NCR⁶R⁷—, with a compound of formula (III) in the presence of a suitable reducing agent such as NaCNBH₃.

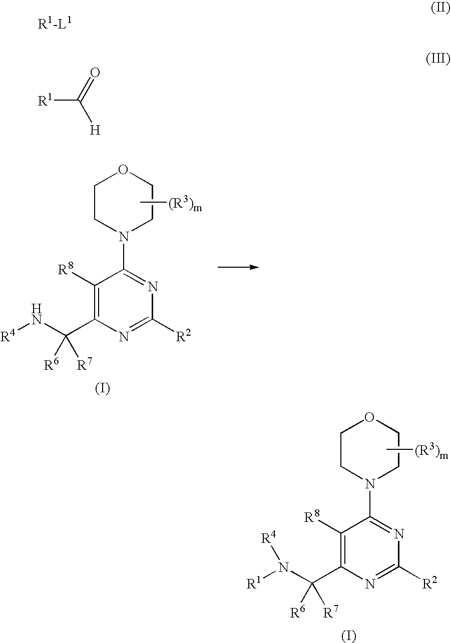

A compound of formula (I), wherein X¹═—S(O)₂CR⁶R⁷—, —SCR⁶R⁷—, —OCR⁶R⁷—, —R⁴NCR⁶R⁷—, —S(O)CR⁶R⁷—, may be prepared by the reaction of a compound of formula (IV), wherein L¹ is a leaving group (such as halo, tosyl, mesyl etc.), with a compound of formula (V) optionally in the presence of a suitable base such as triethylamine and a solvent such as tetrahydrofuran or N,N-dimethylformamide.

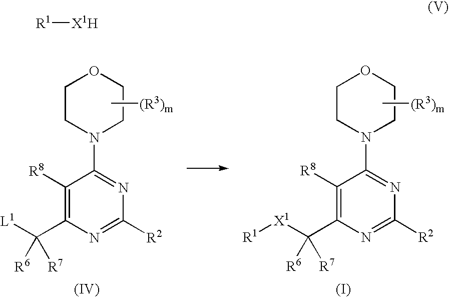

A compound of formula (I), wherein X═—SCR⁶R⁷—, may be prepared by the reaction of a compound of formula (IV), wherein L¹ is a leaving group (such as halo, tosyl, mesyl etc.), with thiourea in a suitable solvent such as ethanol to generate a compound of formula (VI) which is then subsequently reacted with a compound of formula (II) in the presence of a suitable base such as sodium hydroxide and a solvent such as N,N-dimethylformamide.

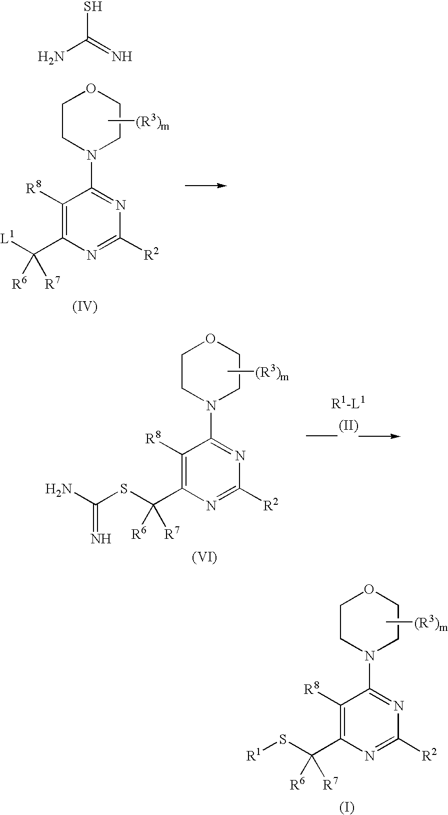

A compound of formula (I), wherein X═—S(O)₂CR⁶R⁷—, may be prepared by the reaction of a compound of formula (VIII) with a compound of formula (VII), wherein R^{6 & 7} is a 2 to 9 membered, optionally substituted, alkylene chain in which 1 carbon may be optionally replaced with O, N or S, and wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

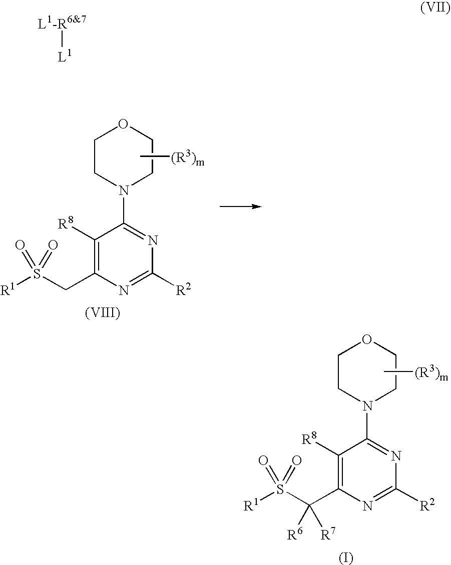

A compound of formula (I), wherein X═—S(O)₂CR⁶R⁷—, may be prepared by the reaction of a compound of formula (VIII) with a compound of formula (IX), wherein R^{6 & 7} is a 2 to 9 membered, optionally substituted, alkylene chain in which 1 carbon may be optionally replaced with O, N or S, and wherein L¹ is a leaving group (such as halo, tosyl, mesyl etc.), and L³ is a group which can be transformed to a suitable leaving group (such as halo, tosyl, mesyl) at a later stage, to give a compound of formula (X) in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide, and subsequently converting L³ to an appropriate leaving group (such as halo, tosyl, mesyl etc.) and then exposing to a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

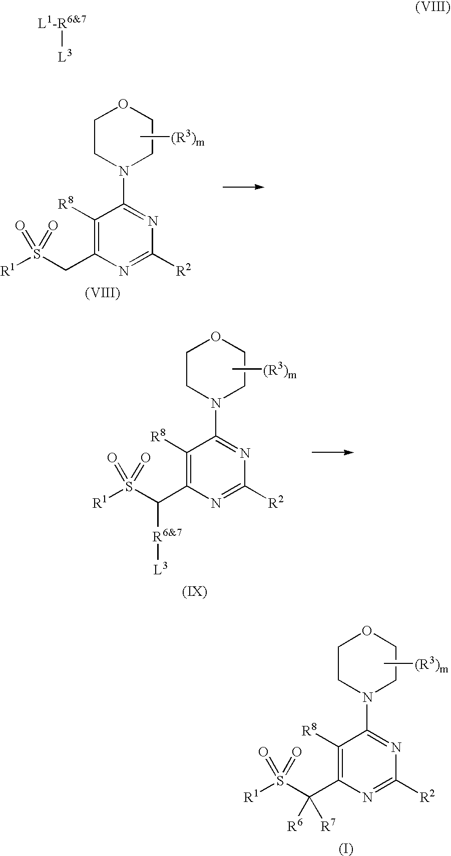

A compound of formula (I), wherein R¹X═HOCR⁶R⁷—, may be prepared by the reaction of a compound of formula (XI), with suitable organometallic reagent of formula (XII) wherein R^{6 & 7} is a 2 to 9 membered, optionally substituted, alkylene chain in which 1 carbon may be optionally replaced with O, N or S, such as the grignard reagent in a suitable solvent.

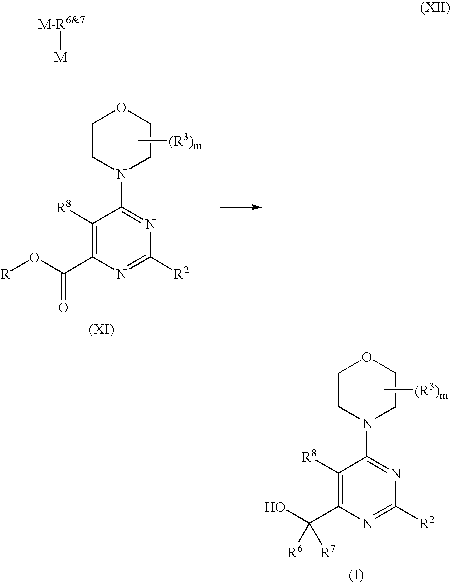

A compound of formula (I), wherein R¹X═HOCR⁶R⁷—, may be prepared by the reaction of a compound of formula (XI), with suitable organometallic reagent of formula (XIII) wherein R^{6 & 7} is a 2 to 9 membered, optionally substituted, alkylene chain in which 1 carbon may be optionally replaced with O, N or S, and M¹ is a group which can be transformed into a suitable organometallic reagent (such as a grignard reagent) at a later date, such as the grignard reagent in a suitable solvent, to give a compound of formula (XIV), and then subsequent conversion of M¹ to a suitable organometallic reagent and subsequent reaction.

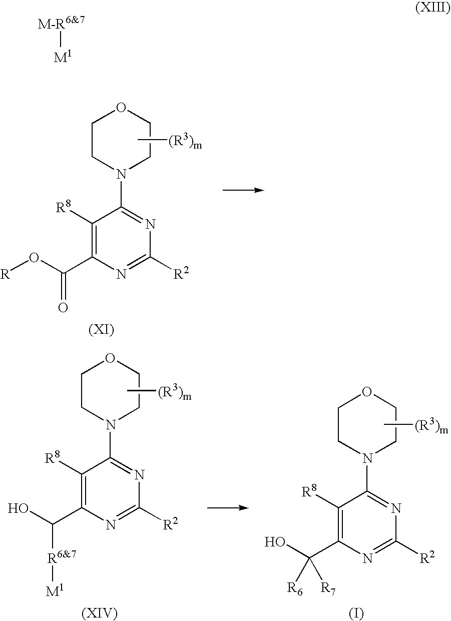

A compound of formula (I) may be prepared from a compound of formula (XV), wherein L² is a leaving group (such as halo, tosyl, mesyl, —SMe, —S(O)₂Me etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (I) may be prepared from a compound of formula (XIII), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

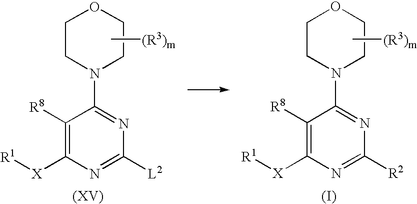

It will be appreciated that a compound of formula (XV) may be transformed into another compound of formula (XV) by techniques such as oxidation, alkylation, reductive amination etc., either listed above or otherwise known in the literature.

A compound of formula (XV), wherein X¹═—S(O)₂CR⁶R⁷—, —SCR⁶R⁷—, —OCR⁶R⁷—, —R⁴NCR⁶R⁷—, —S(O)CR⁶R⁷—, may be prepared by the reaction of a compound of formula (XVI), wherein L¹ is a leaving group (such as halo, tosyl, mesyl etc.), with a compound of formula (V) optionally in the presence of a suitable base such as triethylamine and a solvent such as tetrahydrofuran or N,N-dimethylformamide.

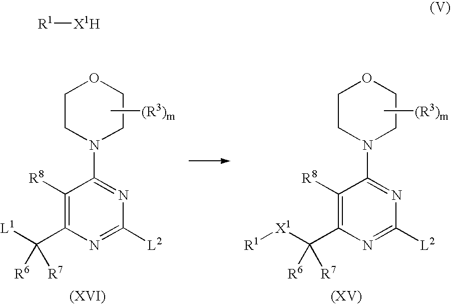

A compound of formula (XV), wherein X═—SCR⁶R⁷—, may be prepared by the reaction of a compound of formula (XVI), wherein L¹ is a leaving group (such as halo, tosyl, mesyl etc.), with thiourea in a suitable solvent such as ethanol to generate a compound of formula (XVII) which is then subsequently reacted with a compound of formula (II) in the presence of a suitable base such as sodium hydroxide and a solvent such as N,N-dimethylformamide.

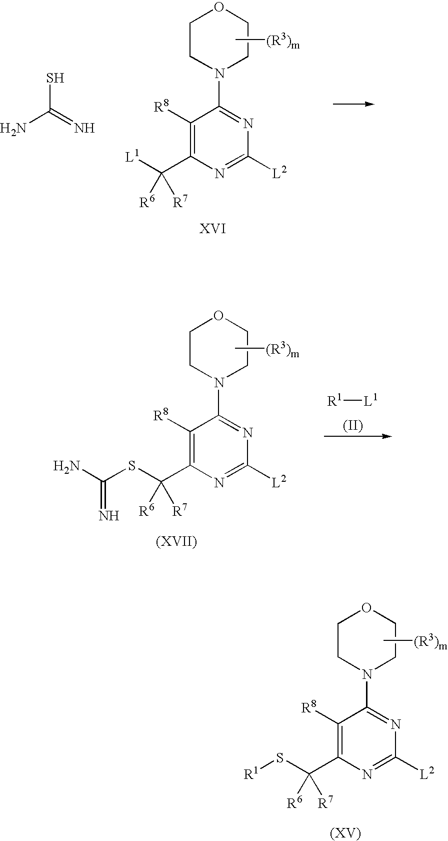

A compound of formula (XV), wherein X═—S(O)₂CR⁶R⁷—, may be prepared by the reaction of a compound of formula (XVIII), wherein X═—S(O)₂CH₂—, with a compound of formula (VII), wherein R^{6 & 7} is a 2 to 9 membered, optionally substituted, alkylene chain in which 1 carbon may be optionally replaced with O, N or S, and wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

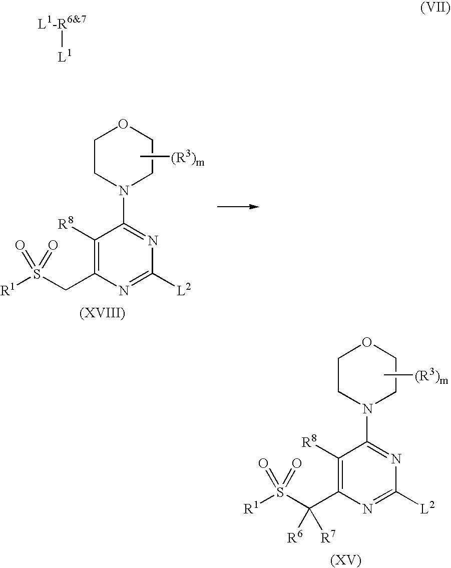

A compound of formula (XV), wherein X═—S(O)₂CR⁶R⁷—, may be prepared by the reaction of a compound of formula (XVIII), wherein X═—S(O)₂CH₂—, with a compound of formula (IX), wherein R^{6 & 7} is a 2 to 9 membered, optionally substituted, alkylene chain in which 1 carbon may be optionally replaced with O, N or S, and wherein L¹ is a leaving group (such as halo, tosyl, mesyl etc.), and L³ is a group which can be transformed to a suitable leaving group (such as halo, tosyl, mesyl) at a later stage, to give a compound of formula (XIX) in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide, and subsequently converting L3 to an appropriate leaving group (such as halo, tosyl, mesyl etc.) and then exposing to a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

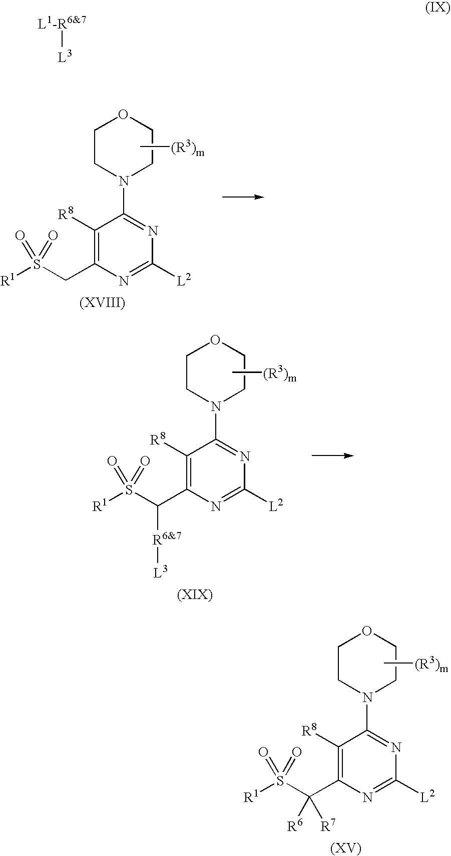

A compound of formula (XV), wherein R¹X═HOCR⁶R⁷—, may be prepared by the reaction of a compound of formula (XX), with suitable organometallic reagent of formula (XII) wherein R^{6 & 7} is a 2 to 9 membered, optionally substituted, alkylene chain in which 1 carbon may be optionally replaced with O, N or S, such as the grignard reagent in a suitable solvent.

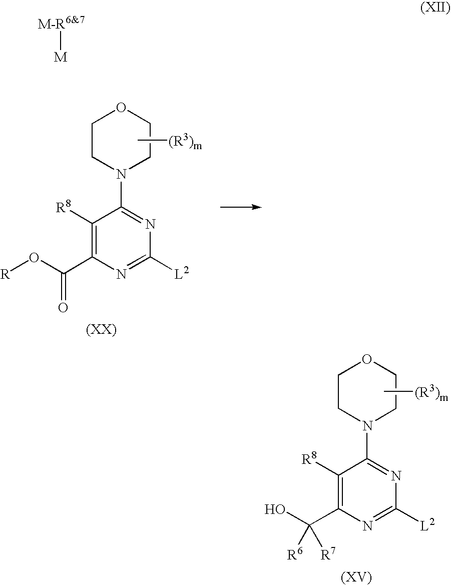

A compound of formula (XV), wherein R¹X═HOCR⁶R⁷—, may be prepared by the reaction of a compound of formula (XX), with suitable organometallic reagent of formula (XIII) wherein R^{6 & 7} is a 2 to 9 membered, optionally substituted, alkylene chain in which 1 carbon may be optionally replaced with O, N or S, and M¹ is a group which can be transformed into a suitable organometallic reagent (such as a grignard reagent) at a later date, such as the grignard reagent in a suitable solvent, to give a compound of formula (XXI), and then subsequent conversion of M¹ to a suitable organometallic reagent and subsequent reaction.

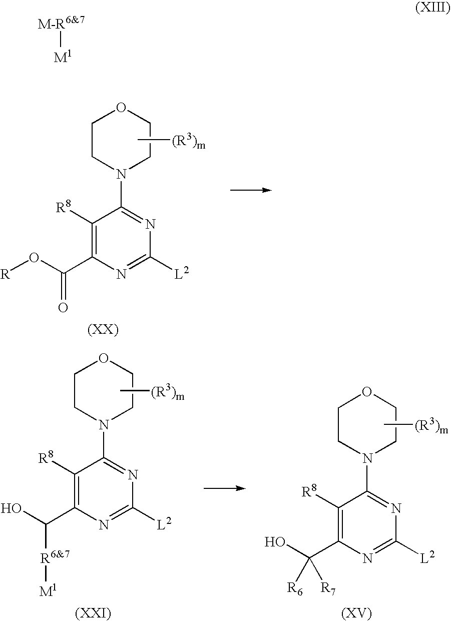

A compound of formula (IV) may be prepared from a compound of formula (XVI), wherein L² is a leaving group (such as halo, tosyl, mesyl, —SMe, —S(O)₂Me etc.) and L¹ is a leaving group (such as halo, tosyl, mesyl etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (IV) may be prepared from a compound of formula (XVI), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

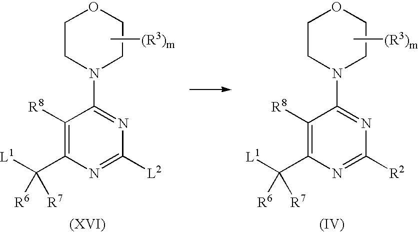

A compound of formula (XI) may be prepared from a compound of formula (XX), wherein L² is a leaving group (such as halo, tosyl, mesyl, —SMe, —S(O)₂Me etc.) and R is a hydrogen or C₁₋₄ alkyl group, with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (XI) may be prepared from a compound of formula (XX), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

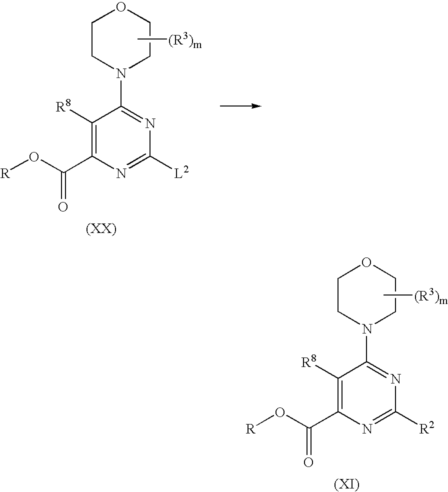

A compound of formula (XXII) may be prepared from a compound of formula (XXIII), wherein L² is a leaving group (such as halo, tosyl, mesyl, —SMe, —S(O)₂Me etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R2 connects to the pyrimidine ring through a nitrogen, oxygen or sulphur atom a compound of formula (XXII) may be prepared from a compound of formula (XXIII), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

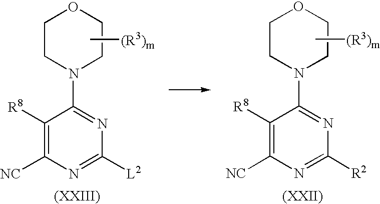

A compound of formula (XXIV) may be prepared from a compound of formula (XXV), wherein L² is a leaving group (such as halo, tosyl, mesyl, —SMe, —S(O)₂Me etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (XXIV) may be prepared from a compound of formula (XXV), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

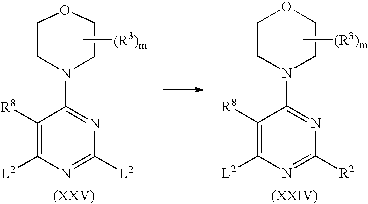

A compound of formula (I), wherein L¹ is a leaving group (such as halo, tosyl, mesyl etc.), may be prepared by the reaction of a compound of formula (XXVI) with a compound of formula (XXVII) optionally in the presence of a suitable base such as triethylamine in a suitable solvent such as N,N-dimethylformamide.

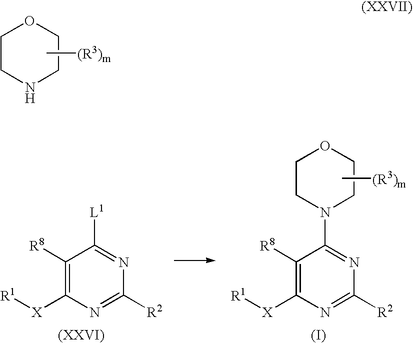

It will be appreciated that a compound of formula (XXV) may be transformed into another compound of formula (XXV) by techniques such as oxidation, alkylation, reductive amination etc., either listed above or otherwise known in the literature.

A compound of formula (IV), wherein L¹ is a leaving group (such as halo, tosyl, mesyl etc.), may be prepared by the reaction of a compound of formula (XXVIII) with a compound of formula (XXVII) optionally in the presence of a suitable base such as triethylamine in a suitable solvent such as N,N-dimethylformamide.

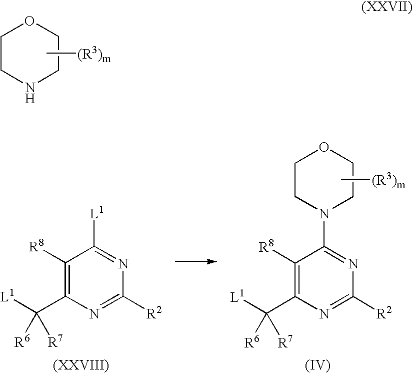

A compound of formula (XI), wherein R is a hydrogen or a C₁₋₄ alkyl group, may be prepared by the reaction of a compound of formula (XXIX), wherein L¹ is a leaving group (such as halo, tosyl, mesyl etc.) with a compound of formula (XXVII) optionally in the presence of a suitable base such as triethylamine in a suitable solvent such as N,N-dimethylformamide.

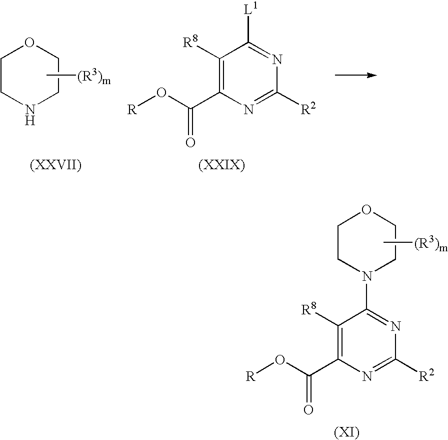

A compound of formula (XXII) may be prepared by the reaction of a compound of formula (XXX), wherein L¹ is a leaving group (such as halo, tosyl, mesyl etc.), with a compound of formula (XXVII) optionally in the presence of a suitable base such as triethylamine in a suitable solvent such as N,N-dimethylformamide.

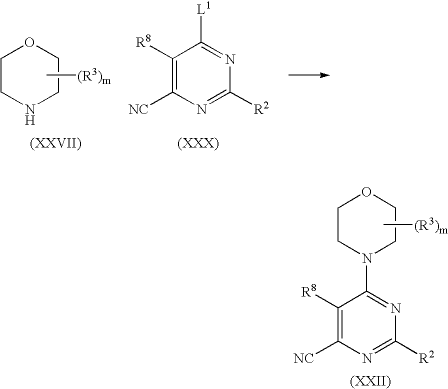

A compound of formula (XXIV), wherein L² is a leaving group (such as halo, tosyl, mesyl, —SMe, —S(O)₂Me etc.), may be prepared by the reaction of a compound of formula (XXXI), wherein L¹ is a leaving group (such as halo, tosyl, mesyl etc.) with a compound of formula (XXVII) optionally in the presence of a suitable base such as triethylamine in a suitable solvent such as N,N-dimethylformamide.

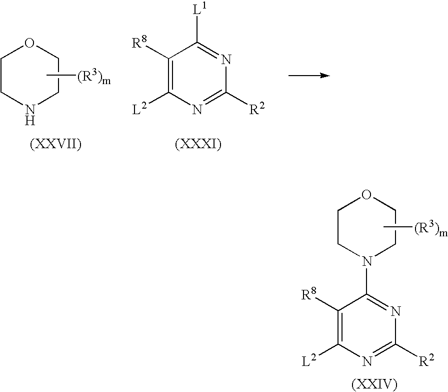

A compound of formula (XV), wherein L² is a leaving group (such as halo, tosyl, mesyl, —SMe, —S(O)₂Me etc.), may be prepared by the reaction of a compound of formula (XXXII), wherein L¹ is a leaving group (such as halo, tosyl, mesyl etc.) with a compound of formula (XXVII) optionally in the presence of a suitable base such as triethylamine in a suitable solvent such as N,N-dimethylformamide.

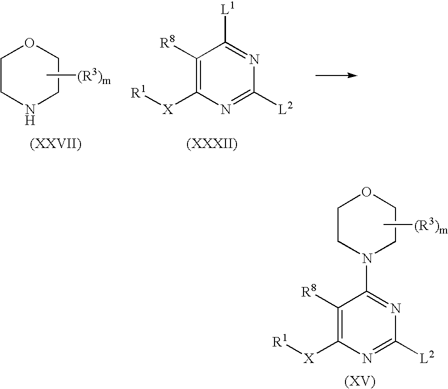

It will be appreciated that a compound of formula (XXXII) may be transformed into another compound of formula (XXXII) by techniques such as oxidation, alkylation, reductive amination etc., either listed above or otherwise known in the literature.

A compound of formula (XVI), wherein L¹ is a leaving group (such as halo, tosyl, mesyl etc.) and L² is a leaving group (such as halo, tosyl, mesyl, —SMe, —S(O)₂Me etc.), may be prepared by the reaction of a compound of formula (XXXIII) with a compound of formula (XXVII) optionally in the presence of a suitable base such as triethylamine in a suitable solvent such as N,N-dimethylformamide.

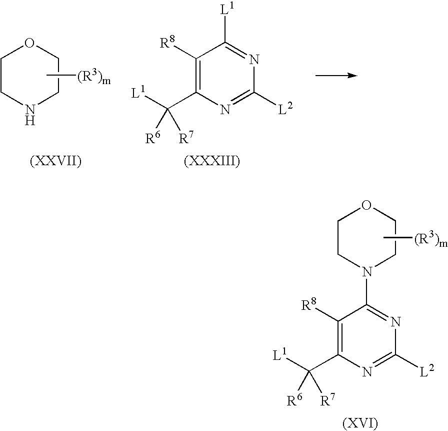

A compound of formula (XX), wherein L² is a leaving group (such as halo, tosyl, mesyl, —SMe, —S(O)₂Me etc.) and R is a hydrogen or a C₁₋₄ alkyl group, may be prepared by the reaction of a compound of formula (XXXIV), wherein L¹ is a leaving group (such as halo, tosyl, mesyl etc.) with a compound of formula (XXVII) optionally in the presence of a suitable base such as triethylamine in a suitable solvent such as N,N-dimethylformamide.

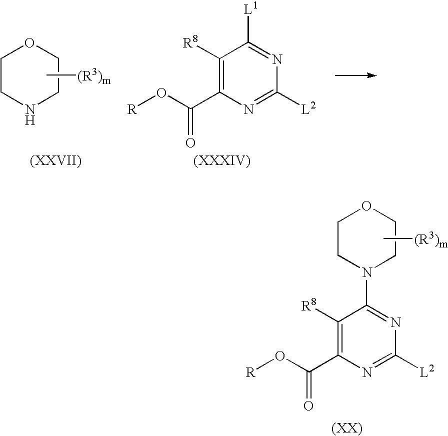

A compound of formula (XXIII), wherein L² is a leaving group (such as halo, tosyl, mesyl, —SMe, —S(O)₂Me etc.), may be prepared by the reaction of a compound of formula (XXXV), wherein L¹ is a leaving group (such as halo, tosyl, mesyl etc.) with a compound of formula (XXVII) optionally in the presence of a suitable base such as triethylamine in a suitable solvent such as N,N-dimethylformamide.

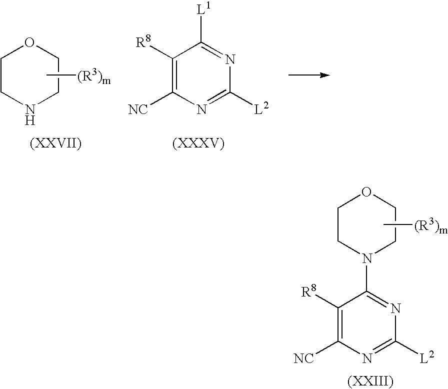

A compound of formula (XXV), wherein L² is a leaving group (such as halo, tosyl, mesyl, —SMe, —S(O)₂Me etc.), may be prepared by the reaction of a compound of formula (XXXVI), wherein L¹ is a leaving group (such as halo, tosyl, mesyl etc.), with a compound of formula (XXVII) optionally in the presence of a suitable base such as triethylamine in a suitable solvent such as N,N-dimethylformamide.

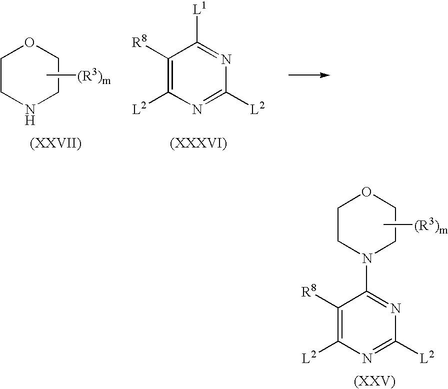

A compound of formula (XXXVII), wherein R¹X═H₂NC(O)—, may be prepared from a compound of formula (XXII) by hydrolysis with, for example, sodium hydroxide in a suitable solvent such as a water ethanol mix.

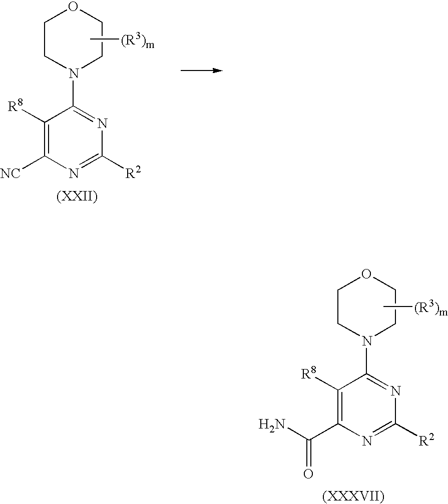

A compound of formula (I), wherein R¹X═H₂NCR⁶R⁷—, may be prepared by the reaction of a compound of formula (XXII), with suitable organometallic reagent of formula (XII) wherein R^{6 & 7} is a 2 to 9 membered, optionally substituted, alkylene chain in which 1 carbon may be optionally replaced with O, N or S, such as the grignard reagent or alkyl lithium reagent in a suitable solvent.

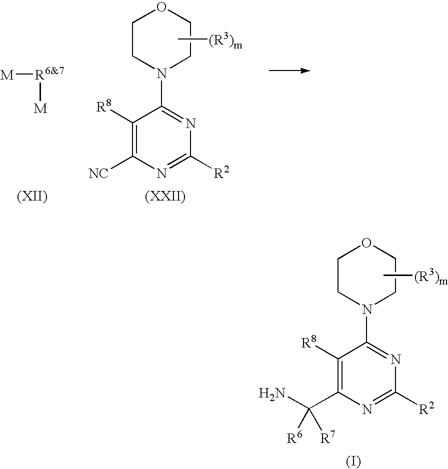

A compound of formula (XV), wherein R¹X═H₂NCR⁶R⁷—, may be prepared by the reaction of a compound of formula (XXIII), with suitable organometallic reagent of formula (XII) wherein R^{6 & 7} is a 2 to 9 membered, optionally substituted, alkylene chain in which 1 carbon may be optionally replaced with O, N or S, such as the grignard reagent or alkyl lithium reagent in a suitable solvent.

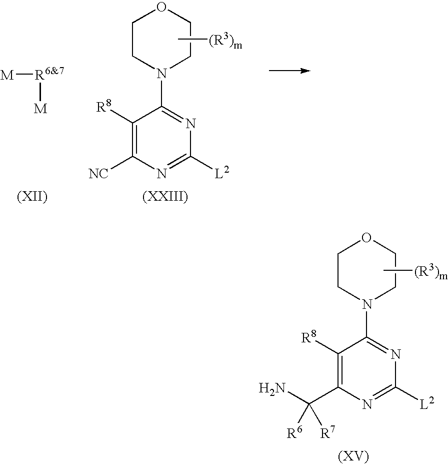

A compound of formula (VIII) may be prepared by the reaction of a compound of formula (XXXVIII), wherein L¹ is a leaving group (such as halo, tosyl, mesyl etc.), with a compound of formula (V), wherein X¹═—S—, —SO₂—, optionally in the presence of a suitable base such as triethylamine and a solvent such as tetrahydrofuran or N,N-dimethylformamide. In the case where X¹═—SH a subsequent oxidation step, for example by using Oxone® at room temperature in a solvent system of water and ethanol, or for example by using 3-chloroperbenzoic acid with dichloromethane as solvent will be required.

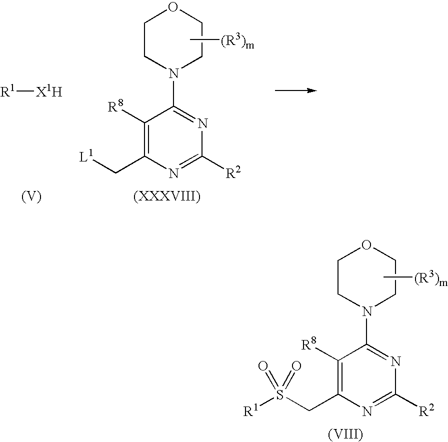

A compound of formula (VIII) may be prepared by the reaction of a compound of formula (XXXVIII), wherein L¹ is a leaving group (such as halo, tosyl, mesyl etc.), with thiourea in a suitable solvent such as ethanol to generate a compound of formula (XXXIX) which is then subsequently reacted with a compound of formula (II) in the presence of a suitable base such as sodium hydroxide and a solvent such as N,N-dimethylformamide, and subsequently oxidised, for example by using Oxone® at room temperature in a solvent system of water and ethanol, or for example by using 3-chloroperbenzoic acid with dichloromethane as solvent.

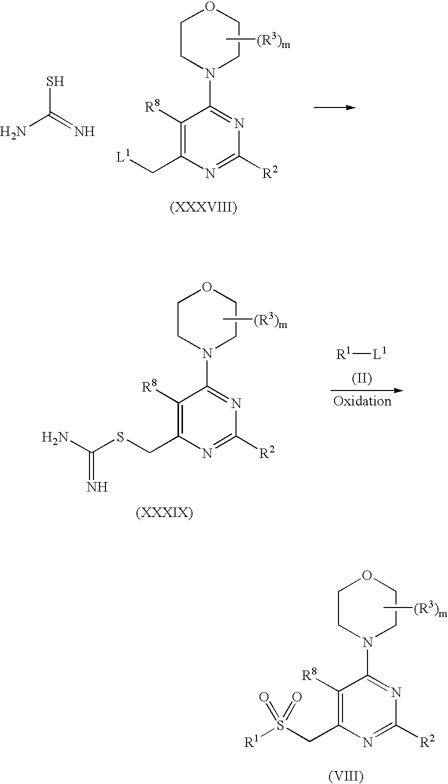

A compound of formula (XVIII), wherein L² is a leaving group (such as halo, tosyl mesyl, —SMe, —S(O)₂Me etc.), may be prepared by the reaction of a compound of formula (XL), wherein L¹ is a leaving group (such as halo, tosyl, mesyl etc.), with a compound of formula (V), wherein X¹═—S—, —SO₂—, optionally in the presence of a suitable base such as triethylamine and a solvent such as tetrahydrofuran or N,N-dimethylformamide. In the case where X¹═—S— a subsequent oxidation step, for example by using Oxone® at room temperature in a solvent system of water and ethanol, or for example by using 3-chloroperbenzoic acid with dichloromethane as solvent will be required.

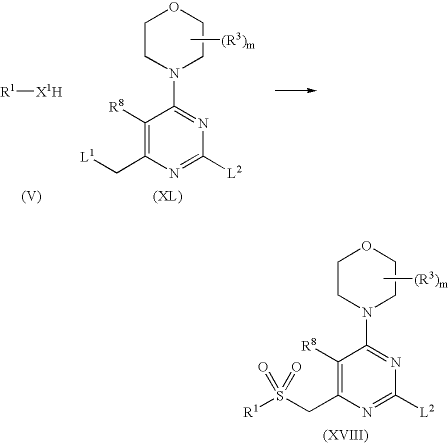

A compound of formula (XVIII), wherein L² is a leaving group (such as halo, tosyl mesyl, —SMe, —S(O)₂Me etc.), may be prepared by the reaction of a compound of formula (XL), wherein L¹ is a leaving group (such as halo, tosyl, mesyl etc.), with thiourea in a suitable solvent such as ethanol to generate a compound of formula (XLI) which is then subsequently reacted with a compound of formula (II) in the presence of a suitable base such as sodium hydroxide and a solvent such as N,N-dimethylformamide, and subsequently oxidised, for example by using Oxone® at room temperature in a solvent system of water and ethanol, or for example by using 3-chloroperbenzoic acid with dichloromethane as solvent.

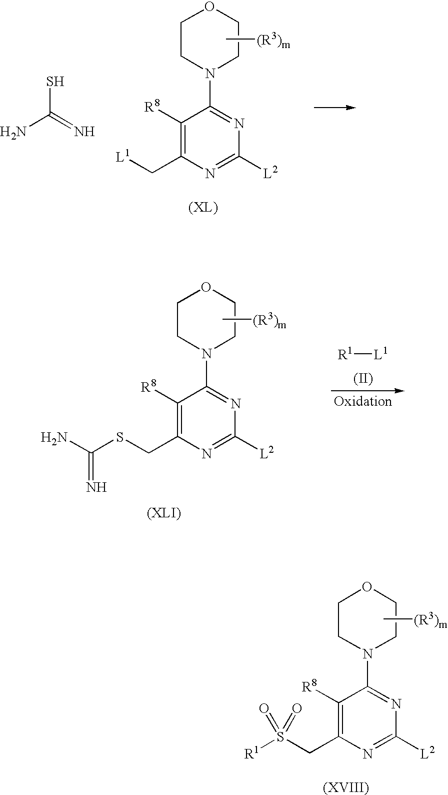

It will be appreciated that the substituent R¹ present in a compound of formula (I) and formula (XV) can be transformed into another substituent R¹ by a series of chemical transformations known in the literature, such as oxidation, reduction, nucleophilic or electrophilic reactions, addition and elimination reactions. An example of such a transformation would be the reaction of a compound of formula (XLII), wherein L² is a leaving group (such as halo, tosyl mesyl, —SMe, —S(O)₂Me etc.), with an electrophile such as dimethyl carbonate in the presence of a base such as sodium hydride in a suitable solvent such as tetrahydrofuran, followed by a reduction, such as the conversion to a mixed anhydride followed by treatment with a hydride source, to give a compound of formula (XLIII)

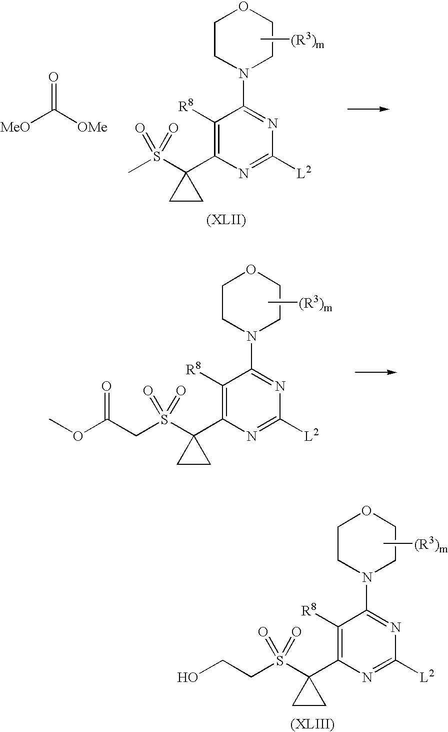

It will be appreciated that the R² group may be introduced and subsequently converted to another group of the formula R² at a subsequent stage in the synthesis using methods known in the literature. For example, but not limited to, an R2 containing an alkyl or aryl amine (which may be suitably protected as, for example, a nitro or t-butoxycarbamate) may be introduced at any stage and then converted, for instance, to a urea by reaction with a suitable isocyanate (or by activation to a suitable group, such as isocyanate or phenoxycarbamate, and subsequent reaction with an amine); or to a thiourea by reaction with a suitable isothiocyanate (or by activation to a suitable group, such as an isothiocyanate, and subsequent reaction with an amine); or to an amide or sulphonamide by reaction with a suitably activated carboxylic acid or sulphonic acid derivative; or by other methods known in the literature.

It will be appreciated that where R⁶ and R⁷, together with the carbon to which they are attached, form a 3-10 membered heterocyclic ring containing a nitrogen atom that the nitrogen atom may be suitably protected (for example a t-butoxycarbamate or benzyl group) and that the protecting group may be removed and if necessary a further reaction performed on the nitrogen (for example an alkylation, reductive amination or amidation) at any stage in the synthesis.

A compound of formula (XLV) may be prepared by the reaction of an amine of formula R¹R⁴NH with a compound of formula (XLIV) in the presence of a suitable coupling agent, such as HATU, or following suitable activation of (XLIV), such as the conversion to an acid chloride.

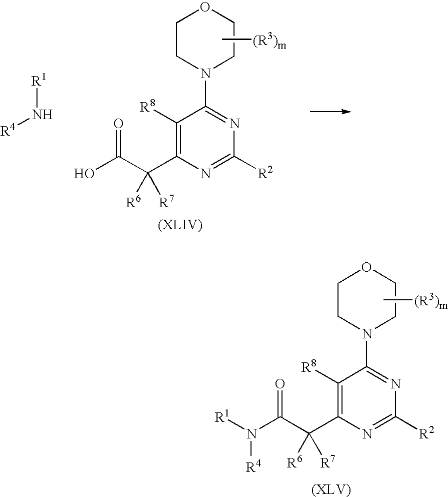

A compound of formula (XLIV) may be prepared from a compound of formula (XLVI) by hydrolysis, for example with sodium hydroxide in a suitable solvent such as an ethanol:water mix.

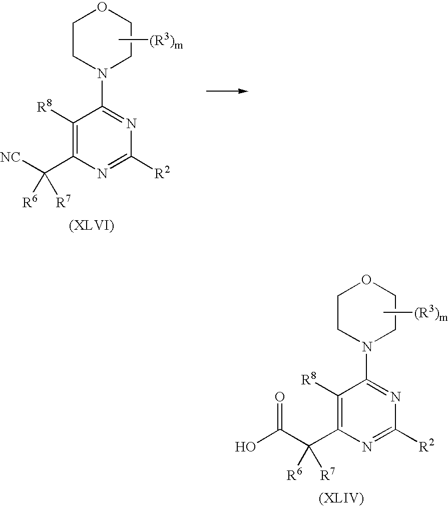

A compound of formula (XLVII) may be prepared by the reaction of an amine of formula R¹R⁴NH with a compound of formula (XLVIII) in the presence of a suitable coupling agent, such as HATU, or following suitable activation of (XLVIII), such as the conversion to an acid chloride.

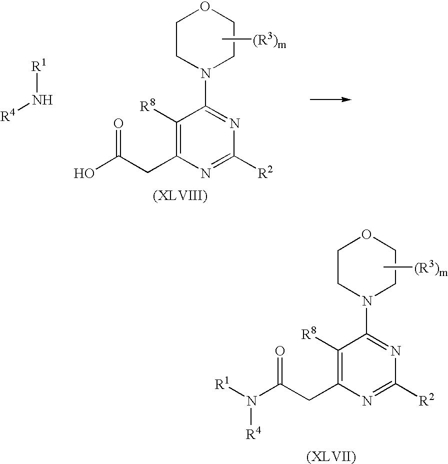

A compound of formula (XLIX) may be prepared from a compound of formula (XLVIII) by hydrolysis, for example with sodium hydroxide in a suitable solvent such as an ethanol:water mix.

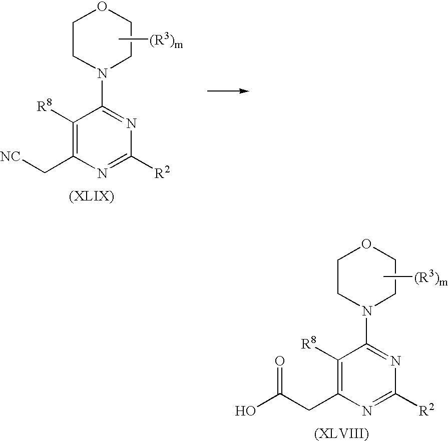

A compound of formula (L), wherein Y═R¹R⁴NC(O)—, ROC(O)—, NC—, may be prepared by the reaction of a compound of formula (VIII) with a compound of formula (LI), wherein R^{6 & 7} is a 2 to 9 membered, optionally substituted, alkylene chain in which 1 carbon may be optionally replaced with O, N or S, and wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

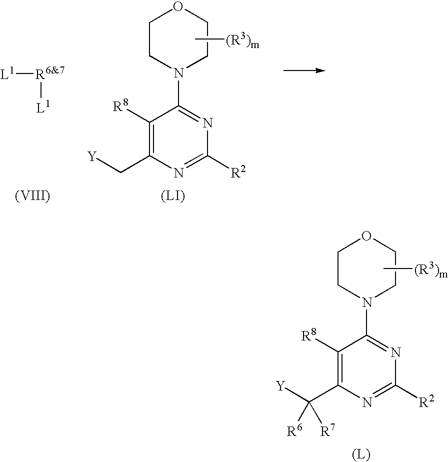

A compound of formula (LI), wherein Y═NC—, HOC(O)—, may be prepared by the reaction of a compound of formula (XXXVIII) with a suitable nucleophile, such as for example sodium cyanide or for example tris(phenylthio)methane anion followed by a suitable hydrolysis.

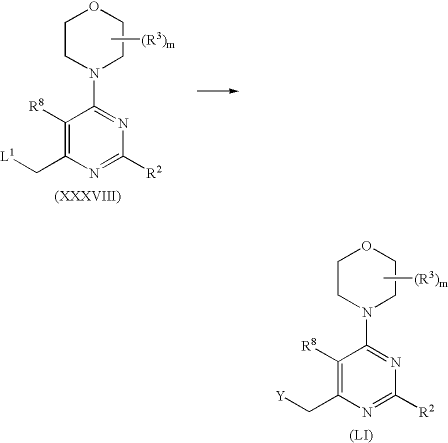

A compound of formula (LII), wherein L² is a leaving group (such as halo, tosyl, mesyl, trifluoromethylsulphonyl, —SMe, —S(O)₂Me etc.), may be prepared by the reaction of an amine of formula R¹R⁴NH with a compound of formula (LIII) in the presence of a suitable coupling agent, such as HATU, or following suitable activation of (LIII), such as the conversion to an acid chloride.

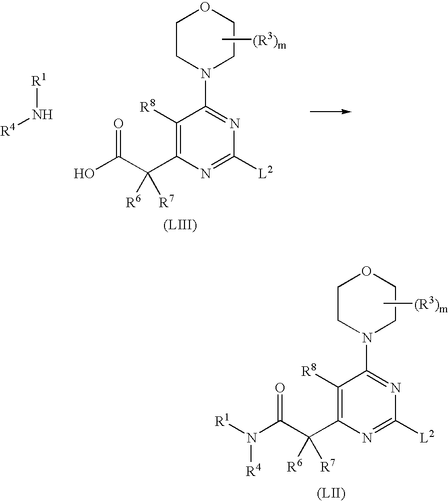

A compound of formula (LIII), wherein L² is a leaving group (such as halo, tosyl, mesyl, trifluoromethylsulphonyl, —SMe, —S(O)₂Me etc.), may be prepared from a compound of formula (LIV) by hydrolysis, for example with sodium hydroxide in a suitable solvent such as an ethanol:water mix.

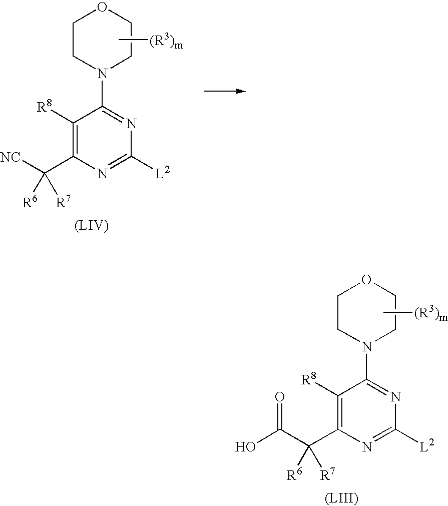

A compound of formula (LV), wherein L² is a leaving group (such as halo, tosyl, mesyl, trifluoromethylsulphonyl, —SMe, —S(O)₂Me etc.), may be prepared by the reaction of an amine of formula R¹R⁴NH with a compound of formula (LVI) in the presence of a suitable coupling agent, such as HATU, or following suitable activation of (LVI), such as the conversion to an acid chloride.

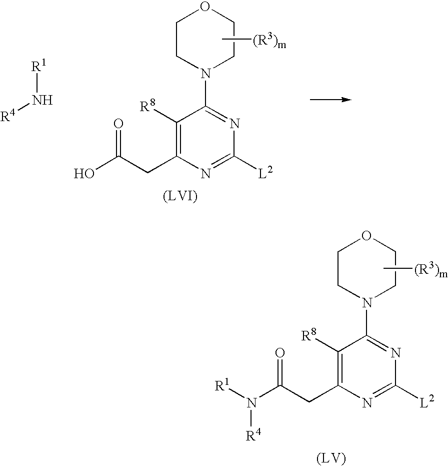

A compound of formula (LVI), wherein L² is a leaving group (such as halo, tosyl, mesyl, trifluoromethylsulphonyl, —SMe, —S(O)₂Me etc.), may be prepared from a compound of formula (LVII) by hydrolysis, for example with sodium hydroxide in a suitable solvent such as an ethanol:water mix.

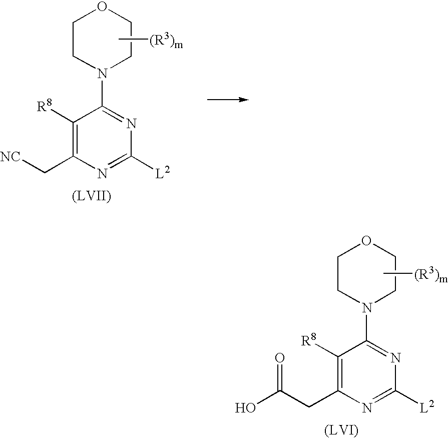

A compound of formula (LVIII), wherein Y═R¹R⁴NC(O)—, ROC(O)—, NC—, and L² is a leaving group (such as halo, tosyl, mesyl, trifluoromethylsulphonyl, —SMe, —S(O)₂Me etc.), may be prepared by the reaction of a compound of formula (VIII) with a compound of formula (LIX), wherein R^{6 & 7} is a 2 to 9 membered, optionally substituted, alkylene chain in which 1 carbon may be optionally replaced with O, N or S, and wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

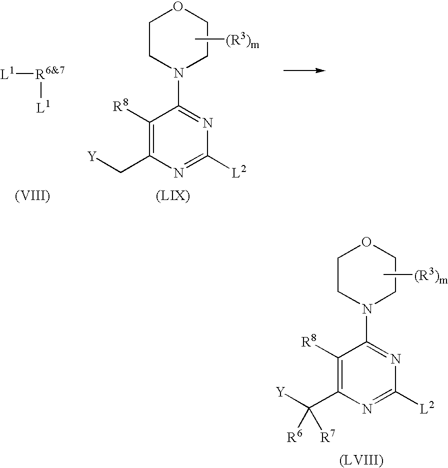

A compound of formula (LIX), wherein Y═NC—, HOC(O)—, and L² is a leaving group (such as halo, tosyl, mesyl, trifluoromethylsulphonyl, —SMe, —S(O)₂Me etc.), may be prepared by the reaction of a compound of formula (XL) with a suitable nucleophile, such as for example sodium cyanide or for example tris(phenylthio)methane anion followed by a suitable hydrolysis.

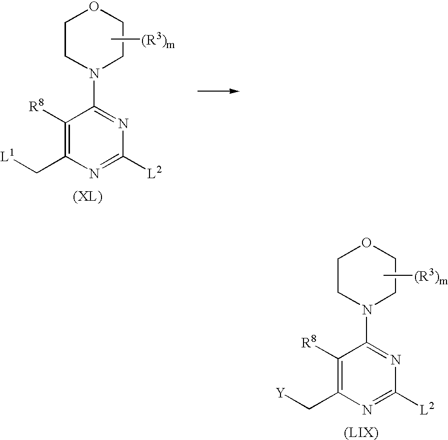

A compound of formula (L), wherein Y═NC—, ROC(O)—, may be prepared by the reaction of a compound of formula (XXVII) with a compound of formula (LX), wherein L² is a leaving group (such as halo, tosyl, mesyl, trifluoromethylsulphonyl, —SMe, —S(O)₂Me etc.), optionally in the presence of a suitable base such as triethylamine in a suitable solvent such as N,N-dimethylformamide.

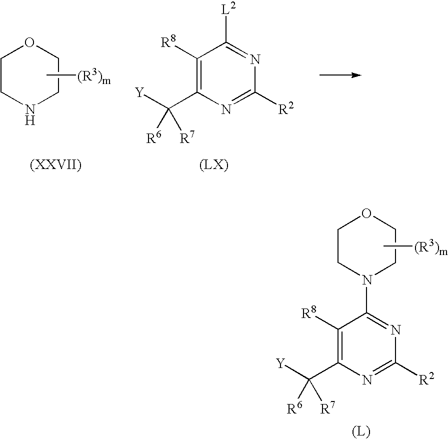

A compound of formula (LX), wherein Y═NC—, ROC(O)—, and L² is a leaving group (such as halo, tosyl, mesyl, trifluoromethylsulphonyl, —SMe, —S(O)₂Me etc.) may be prepared by the reaction of a compound of formula (LXI) with a compound of formula (LXII) and subsequent conversion of the OH group to a suitable leaving group, such as by reaction with N-phenyltrifluoromethanesulfonimide in the presence of a suitable base such as DBU and a suitable solvent such as dichloromethane.

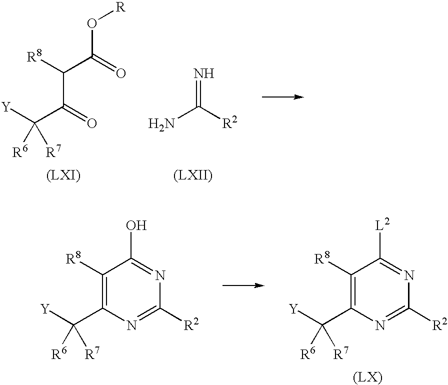

A compound of formula (LI), wherein Y═NC—, ROC(O)—, may be prepared by the reaction of a compound of formula (XXVII) with a compound of formula (LXIII), wherein L² is a leaving group (such as halo, tosyl, mesyl, trifluoromethylsulphonyl, —SMe, —S(O)₂Me etc.), optionally in the presence of a suitable base such as triethylamine in a suitable solvent such as N,N-dimethylformamide.

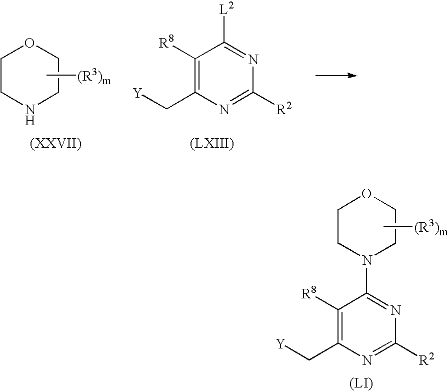

A compound of formula (LXIII), wherein Y═NC—, ROC(O)—, and L² is a leaving group (such as halo, tosyl, mesyl, trifluoromethylsulphonyl, —SMe, —S(O)₂Me etc.) may be prepared by the reaction of a compound of formula (LXIV) with a compound of formula (LXII) and subsequent conversion of the OH group to a suitable leaving group, such as by reaction with N-phenyltrifluoromethanesulfonimide in the presence of a suitable base such as DBU and a suitable solvent such as dichloromethane.

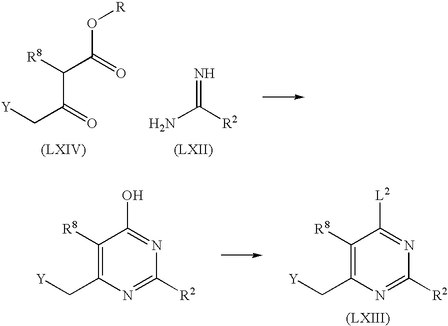

A compound of formula (LVIII), wherein Y═NC—, ROC(O)—, and L² is a leaving group (such as halo, tosyl, mesyl, trifluoromethylsulphonyl, —SMe, —S(O)₂Me etc.) may be prepared by the reaction of a compound of formula (XXVII) with a compound of formula (LXV), wherein L² is a leaving group (such as halo, tosyl, mesyl, trifluoromethylsulphonyl, —SMe, —S(O)₂Me etc.), optionally in the presence of a suitable base such as triethylamine in a suitable solvent such as N,N-dimethylformamide.

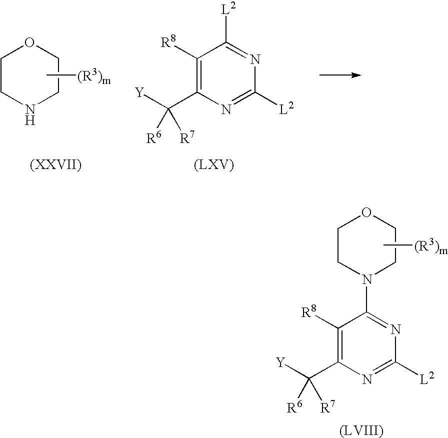

A compound of formula (LXV), wherein Y═NC—, ROC(O)—, and L² is a leaving group (such as halo, tosyl, mesyl, trifluoromethylsulphonyl, —SMe, —S(O)₂Me etc.) may be prepared by the reaction of a compound of formula (LXI) with a compound of formula (LXVI) and subsequent conversion of the OH group to a suitable leaving group, such as by reaction with N-phenyltrifluoromethanesulfonimide in the presence of a suitable base such as DBU and a suitable solvent such as dichloromethane.

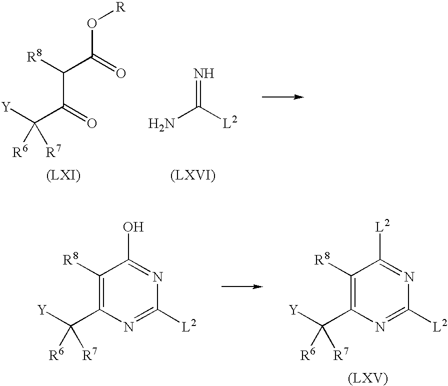

A compound of formula (LIX), wherein Y═NC—, ROC(O)—, and L² is a leaving group (such as halo, tosyl, mesyl, trifluoromethylsulphonyl, —SMe, —S(O)₂Me etc.) may be prepared by the reaction of a compound of formula (XXVII) with a compound of formula (LXVII), wherein L² is a leaving group (such as halo, tosyl, mesyl, trifluoromethylsulphonyl, —SMe, —S(O)₂Me etc.), optionally in the presence of a suitable base such as triethylamine in a suitable solvent such as N,N-dimethylformamide.

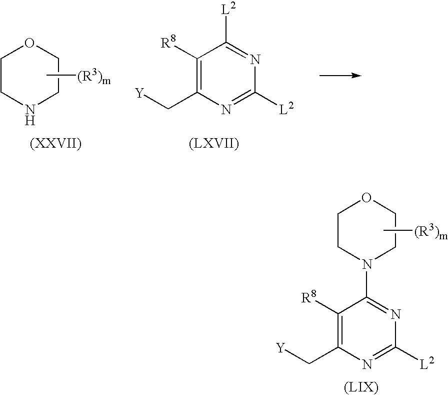

A compound of formula (LXVII), wherein Y═NC—, ROC(O)—, and L² is a leaving group (such as halo, tosyl, mesyl, trifluoromethylsulphonyl, —SMe, —S(O)₂Me etc.) may be prepared by the reaction of a compound of formula (LXIV) with a compound of formula (LXVI) and subsequent conversion of the OH group to a suitable leaving group, such as by reaction with N-phenyltrifluoromethanesulfonimide in the presence of a suitable base such as DBU and a suitable solvent such as dichloromethane.

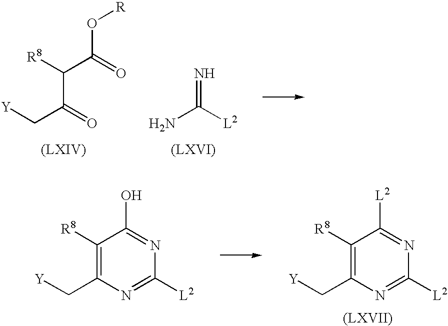

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. For example compounds of formula (I) may be converted into further compounds of formula (I) by standard aromatic substitution reactions or by conventional functional group modifications. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Many of the intermediates defined herein are novel and these are provided as a further feature of the invention.

### Biological Assays

The following assays can be used to measure the effects of the compounds of the present invention as mTOR kinase inhibitors, as PI3 kinase inhibitors, as inhibitors in vitro of the activation of PI3 kinase signalling pathways and as inhibitors in vitro of the proliferation of MDA-MB-468 human breast adenocarcinoma cells.

(a)(i) In Vitro mTOR Kinase Assay

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin (G418 sulfate; Invitrogen Limited, UK Catalogue No. 10131-027). Following expression in the mammalian HEK293 cell line, expressed protein was purified using the FLAG epitope tag using standard purification techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one). A 30 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH 7.4 buffer (50 mM), EGTA (0.1 mM), bovine serum albumin (0.5 mg/mL), DTT (1.25 mM) and manganese chloride (10 mM)] was agitated at room temperature for 90 minutes.

Control wells that produced a maximum signal corresponding to maximum enzyme activity were created by using 5% DMSO instead of test compound. Control wells that produced a minimum signal corresponding to fully inhibited enzyme were created by adding EDTA (83 mM) instead of test compound. These assay solutions were incubated for 2 hours at room temperature.

Each reaction was stopped by the addition of 10 μl of a mixture of EDTA (50 mM), bovine serum albumin (BSA; 0.5 mg/mL) and Tris-HCl pH 7.4 buffer (50 mM) containing p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left for about 20 hours at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

(a)(ii) In Vitro mTOR Kinase Assay (Echo)

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin (G418 sulfate; Invitrogen Limited, UK Catalogue No. 1013 1-027). Following expression in the mammalian HEK293 cell line, expressed protein was purified using the FLAG epitope tag using standard purification techniques.

Test compounds were prepared as 10 mM stock solutions in DMSO and diluted in into water DMSO as required to give a range of final assay concentrations. Aliquots (120 nl 2 μl) of each compound dilution were acoustically dispensed placed using a Labcyte Echo 550 into a well of a Greiner 384-well low volume (LV) white polystyrene plate (Greiner Bio-one). A 1230 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH 7.4 buffer (50 mM), EGTA (0.1 mM), bovine serum albumin (0.5 mg/mL), DTT (1.25 mM) and manganese chloride (10 mM)] was incubated at room temperature for 12090 minutes.

Control wells that produced a maximum signal corresponding to maximum enzyme activity were created by using 1005% DMSO instead of test compound. Control wells that produced a minimum signal corresponding to fully inhibited enzyme were created by adding LY294002EDTA (100 uM 83 mM) compound. These assay solutions were incubated for 2 hours at room temperature.

Each reaction was stopped by the addition of 510 μl of a mixture of EDTA (50 mM), bovine serum albumin (BSA; 0.5 mg/mL) and Tris-HCl pH 7.4 buffer (50 mM) containing p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left overnight at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

(b)(i) In Vitro PI3K Enzyme Assay

The assay used AlphaScreen technology (Gray et al., Analytical Biochemistry, 2003, 313: 234-245) to determine the ability of test compounds to inhibit phosphorylation by recombinant Type I PI3K enzymes of the lipid PI(4,5)P2.

DNA fragments encoding human PI3K catalytic and regulatory subunits were isolated from cDNA libraries using standard molecular biology and PCR cloning techniques. The selected DNA fragments were used to generate baculovirus expression vectors. In particular, full length DNA of each of the p110α, p110β and p110δ Type Ia human PI3K p110 isoforms (EMBL Accession Nos. HSU79143, S67334, Y10055 for p110α, p110β and p110δ respectively) were sub-cloned into a pDEST10 vector (Invitrogen Limited, Fountain Drive, Paisley, UK). The vector is a Gateway-adapted version of Fastbac1 containing a 6-His epitope tag. A truncated form of Type Ib human PI3K p110γ isoform corresponding to amino acid residues 144-1102 (EMBL Accession No. X8336A) and the full length human p85α regulatory subunit (EMBL Accession No. HSP13KIN) were also sub-cloned into pFastBac1 vector containing a 6-His epitope tag. The Type Ia p110 constructs were co-expressed with the p85α regulatory subunit. Following expression in the baculovirus system using standard baculovirus expression techniques, expressed proteins were purified using the His epitope tag using standard purification techniques.

DNA corresponding to amino acids 263 to 380 of human general receptor for phosphoinositides (Grp1) PH domain was isolated from a cDNA library using standard molecular biology and PCR cloning techniques. The resultant DNA fragment was sub-cloned into a pGEX 4T1 E. coli expression vector containing a GST epitope tag (Amersham Pharmacia Biotech, Rainham, Essex, UK) as described by Gray et al, Analytical Biochemistry, 2003, 313: 234-245). The GST-tagged Grp1 PH domain was expressed and purified using standard techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one, Brunel Way, Stonehouse, Gloucestershire, UK Catalogue No. 784075). A mixture of each selected recombinant purified PI3K enzyme (15 ng), DiC8-PI(4,5)P2 substrate (40 μM; Cell Signals Inc., Kinnear Road, Columbus, USA, Catalogue No. 901), adenosine triphosphate (ATP; 4 μM) and a buffer solution [comprising Tris-HCl pH7.6 buffer (40 mM, 10 μl), 3-[(3-cholamidopropyl)dimethylammonio]-1-propanesulfonate (CHAPS; 0.04%), dithiothreitol (DTT; 2 mM) and magnesium chloride (10 mM)] was agitated at room temperature for 20 minutes.

Control wells that produced a minimum signal corresponding to maximum enzyme activity were created by using 5% DMSO instead of test compound. Control wells that produced a maximum signal corresponding to fully inhibited enzyme were created by adding wortmannin (6 μM; Calbiochem/Merck Bioscience, Padge Road, Beeston, Nottingham, UK, Catalogue No. 681675) instead of test compound. These assay solutions were also agitated for 20 minutes at room temperature.

Each reaction was stopped by the addition of 10 μl of a mixture of EDTA (100 mM), bovine serum albumin (BSA, 0.045%) and Tris-HCl pH 7.6 buffer (40 mM).

Biotinylated-DiC8-PI(3,4,5)P3 (50 nM; Cell Signals Inc., Catalogue No. 107), recombinant purified GST-Grp1 PH protein (2.5 nM) and AlphaScreen Anti-GST donor and acceptor beads (100 ng; Packard Bioscience Limited, Station Road, Pangbourne, Berkshire, UK, Catalogue No. 6760603M) were added and the assay plates were left for about 5 to 20 hours at room temperature in the dark. The resultant signals arising from laser light excitation at 680 nm were read using a Packard AlphaQuest instrument.

PI(3,4,5)P3 is formed in situ as a result of P13K mediated phosphorylation of PI(4,5)P2. The GST-Grp1 PH domain protein that is associated with AlphaScreen Anti-GST donor beads forms a complex with the biotinylated PI(3,4,5)P3 that is associated with Alphascreen Streptavidn acceptor beads. The enymatically-produced PI(3,4,5)P3 competes with biotinylated PI(3,4,5)P3 for binding to the PH domain protein. Upon laser light excitation at 680 nm, the donor bead:acceptor bead complex produces a signal that can be measured. Accordingly, PI3K enzme activity to form PI(3,4,5)P3 and subsequent competition with biotinylated PI(3,4,5)P3 results in a reduced signal. In the presence of a PI3K enzyme inhibitor, signal strength is recovered.

PI3K enzyme inhibition for a given test compound was expressed as an IC₅₀ value.

(b)(ii) In Vitro PI3K Enzyme Assay (Echo)

The assay used AlphaScreen technology (Gray et al., Analytical Biochemistry, 2003, 313: 234-245) to determine the ability of test compounds to inhibit phosphorylation by recombinant Type I PI3K enzymes of the lipid PI(4,5)P2.

DNA fragments encoding human PI3K catalytic and regulatory subunits were isolated from cDNA libraries using standard molecular biology and PCR cloning techniques. The selected DNA fragments were used to generate baculovirus expression vectors. In particular, full length DNA of each of the p110α, p110β and p110δ Type Ia human PI3K p110 isoforms (EMBL Accession Nos. HSU79143, S67334, Y10055 for p110α, p110β and p110δ respectively) were sub-cloned into a pDEST10 vector (Invitrogen Limited, Fountain Drive, Paisley, UK). The vector is a Gateway-adapted version of Fastbac1 containing a 6-His epitope tag. A truncated form of Type Ib human PI3K p110γ isoform corresponding to amino acid residues 144-1102 (EMBL Accession No. X8336A) and the full length human p85α regulatory subunit (EMBL Accession No. HSP13KIN) were also sub-cloned into pFastBac1 vector containing a 6-His epitope tag. The Type Ia p110 constructs were co-expressed with the p85α regulatory subunit. Following expression in the baculovirus system using standard baculovirus expression techniques, expressed proteins were purified using the His epitope tag using standard purification techniques.

DNA corresponding to amino acids 263 to 380 of human general receptor for phosphoinositides (Grp1) PH domain was isolated from a cDNA library using standard molecular biology and PCR cloning techniques. The resultant DNA fragment was sub-cloned into a pGEX 4T1 E. coli expression vector containing a GST epitope tag (Amersham Pharmacia Biotech, Rainham, Essex, UK) as described by Gray et al., Analytical Biochemistry, 2003, 313: 234-245). The GST-tagged Grp1 PH domain was expressed and purified using standard techniques.

Test compounds were prepared as 10 mM stock solutions in DMSO and diluted in DMSO to water as required to give a range of final assay concentrations. Aliquots (120 nl 2 μl) of each compound dilution were acoustically dispensed using a Labcyte Echo 550 placed into a well of a Greiner 384-well low volume (LV) white polystyrene plate (Greiner Bio-one, Brunel Way, Stonehouse, Gloucestershire, UK Catalogue No. 784075). A mixture of each selected recombinant purified PI3K enzyme (15 ng), DiC8-PI(4,5)P2 substrate (40 μM; Cell Signals Inc., Kinnear Road, Columbus, USA, Catalogue No. 901), adenosine triphosphate (ATP; 4 μM) and a buffer solution [comprising Tris-HCl pH 7.6 buffer (40 mM, 10 μl), 3-[(3-cholamidopropyl)dimethylammonio]-1-propanesulfonate (CHAPS; 0.04%), dithiothreitol (DTT; 2 mM) and magnesium chloride (10 mM)] was agitatedincubated at room temperature for 20 minutes.

Control wells that produced a minimum signal corresponding to maximum enzyme activity were created by using 1005% DMSO instead of test compound. Control wells that produced a maximum signal corresponding to fully inhibited enzyme were created by adding Wwortmannin (6 μM; Calbiochem/Merck Bioscience, Padge Road, Beeston, Nottingham, UK, Catalogue No. 681675) instead of test compound. These assay solutions were also incubatedagitated for 20 minutes at room temperature.

Each reaction was stopped by the addition of 10 10 μl of a mixture of EDTA (100 mM), bovine serum albumin (BSA, 0.045%) and Tris-HCl pH 7.6 buffer (40 mM).

Biotinylated-DiC8-PI(3,4,5)P3 (50 nM; Cell Signals Inc., Catalogue No. 107), recombinant purified GST-Grp1 PH protein (2.5 nM) and AlphaScreen Anti-GST donor and acceptor beads (100 ng; Packard Bioscience Limited, Station Road, Pangbourne, Berkshire, UK, Catalogue No. 6760603M) were added and the assay plates were left for about 5 to overnight 20 hours at room temperature in the dark. The resultant signals arising from laser light excitation at 680 nm were read using a Packard AlphaQuest instrument.

PI(3,4,5)P3 is formed in situ as a result of PI3K mediated phosphorylation of PI(4,5)P2. The GST-Grp1 PH domain protein that is associated with AlphaScreen Anti-GST donor beads forms a complex with the biotinylated PI(3,4,5)P3 that is associated with Alphascreen Streptavidn acceptor beads. The enymatically-produced PI(3,4,5)P3 competes with biotinylated PI(3,4,5)P3 for binding to the PH domain protein. Upon laser light excitation at 680 nm, the donor bead:acceptor bead complex produces a signal that can be measured. Accordingly, PI3K enzme activity to form PI(3,4,5)P3 and subsequent competition with biotinylated PI(3,4,5)P3 results in a reduced signal. In the presence of a PI3K enzyme inhibitor, signal strength is recovered.

PI3K enzyme inhibition for a given test compound was expressed as an IC₅₀ value.

(c) In Vitro phospho-Ser473 Akt Assay

This assay determines the ability of test compounds to inhibit phosphorylation of Serine 473 in Akt as assessed using Acumen Explorer technology (Acumen Bioscience Limited), a plate reader that can be used to rapidly quantitate features of images generated by laser-scanning.

A MDA-MB-468 human breast adenocarcinoma cell line (LGC Promochem, Teddington, Middlesex, UK, Catalogue No. HTB-132) was routinely maintained at 37° C. with 5% CO₂ up to a confluency of 70-90% in DMEM containing 10% heat-inactivated FCS and 1% L-glutamine.

For the assay, the cells were detached from the culture flask using ‘Accutase’ (Innovative Cell Technologies Inc., San Diego, Calif., USA; Catalogue No. AT104) using standard tissue culture methods and resuspended in media to give 1.7×10⁵ cells per mL. Aliquots (90 μl) were seeded into each of the inner 60 wells of a black Packard 96 well plate (PerkinElmer, Boston, Mass., USA; Catalogue No. 6005182) to give a density of ˜15000 cells per well. Aliquots (90 μl) of culture media were placed in the outer wells to prevent edge effects. The cells were incubated overnight at 37° C. with 5% CO₂ to allow them to adhere.

On day 2, the cells were treated with test compounds and incubated for 2 hours at 37° C. with 5% CO₂. Test compounds were prepared as 10 mM stock solutions in DMSO and serially diluted as required with growth media to give a range of concentrations that were 10-fold the is required final test concentrations. Aliquots (10 μl) of each compound dilution were placed in a well (in triplicate) to give the final required concentrations. As a minimum reponse control, each plate contained wells having a final concentration of 100 μM LY294002 (Calbiochem, Beeston, UK, Catalogue No. 440202). As a maximum response control, wells contained 1% DMSO instead of test compound. Following incubation, the contents of the plates were fixed by treatment with a 1.6% aqueous formaldehyde solution (Sigma, Poole, Dorset, UK, Catalogue No. F1635) at room temperature for 1 hour.

All subsequent aspiration and wash steps were carried out using a Tecan 96 well plate washer (aspiration speed 10 mm/sec). The fixing solution was removed and the contents of the plates were washed with phosphate-buffered saline (PBS; 50 μl; Gibco, Catalogue No. 10010015). The contents of the plates were treated for 10 minutes at room temperature with an aliquot (50 μl) of a cell permeabilisation buffer consisting of a mixture of PBS and 0.5% Tween-20. The ‘permeabilisation’ buffer was removed and non-specific binding sites were blocked by treatment for 1 hour at room temperature of an aliquot (50 μl) of a blocking buffer consisting of 5% dried skimmed milk [‘Marvel’ (registered trade mark); Premier Beverages, Stafford, GB] in a mixture of PBS and 0.05% Tween-20. The ‘blocking’ buffer was removed and the cells were incubated for 1 hour at room temperature with rabbit anti phospho-Akt (Ser473) antibody solution (50 μl per well; Cell Signalling, Hitchin, Herts, U.K., Catalogue No 9277) that had been diluted 1:500 in ‘blocking’ buffer. Cells were washed three times in a mixture of PBS and 0.05% Tween-20. Subsequently, cells were incubated for 1 hour at room temperature with Alexafluor488 labelled goat anti-rabbit IgG (50 μl per well; Molecular Probes, Invitrogen Limited, Paisley, UK, Catalogue No. A11008) that had been diluted 1:500 in ‘blocking’ buffer. Cells were washed 3 times with a mixture of PBS and 0.05% Tween-20. An aliquot of PBS (50 μl) was added to each well and the plates were sealed with black plate sealers and the fluorescence signal was detected and analysed.

Fluorescence dose response data obtained with each compound were analysed and the degree of inhibition of Serine 473 in Akt was expressed as an IC₅₀ value.

### (d) In Vitro MDA-MB-468 Human Breast Adenocarcinoma Proliferation Assay

This assay determines the ability of test compounds to inhibit cell proliferation as assessed using Cellomics Arrayscan technology. A MDA-MB-468 human breast adenocarcinoma cell line (LGC Promochem, Catalogue No. HTB-132) was routinely maintained as described in Biological Assay (b) herein.

For the proliferation assay, the cells were detached from the culture flask using Accutase and seeded into the inner 60 wells of a black Packard 96 well plate at a density of 8000 cells per well in 100 μl of complete growth media. The outer wells contained 100 μl of sterile PBS. The cells were incubated overnight at 37° C. with 5% CO₂ to allow them to adhere.

On day 2, the cells were treated with test compounds and incubated for 48 hours at 37° C. with 5% CO₂. Test compounds were prepared as 10 mM stock solutions in DMSO and serially diluted as required with growth media to give a range of test concentrations. Aliquots (50 μl) of each compound dilution were placed in a well and the cells were incubated for 2 days at 37° C. with 5% CO₂. Each plate contained control wells without test compound.

On day 4, BrdU labelling reagent (Sigma, Catalogue No. B9285) at a final dilution of 1:1000 was added and the cells were incubated for 2 hours at 37° C. The medium was removed and the cells in each well were fixed by treatment with 100 μl of a mixture of ethanol and glacial acetic acid (90% ethanol, 5% glacial acetic acid and 5% water) for 30 minutes at room temperature. The cells in each well were washed twice with PBS (100 μl). Aqueous hydrochloric acid (2M, 100 μl) was added to each well. After 20 minutes at room temperature, the cells were washed twice with PBS. Hydrogen peroxide (3%, 50 μl; Sigma, Catalogue No. H1009) was added to each well. After 10 minutes at room temperature, the wells were washed again with PBS.

BrdU incorporation was detected by incubation for 1 hour at room temperature with mouse anti-BrdU antibody (50 μl; Caltag, Burlingame, Calif., US; Catalogue No. MD5200) that was diluted 1:40 in PBS containing 1% BSA and 0.05% Tween-20. Unbound antibody was removed with two washes of PBS. For visualisation of incorporated BrdU, the cells were treated for 1 hour at room temperature with PBS (50 μl) and 0.05% Tween-20 buffer containing a 1:1000 dilution of Alexa fluor 488-labelled goat anti-mouse IgG. For visualisation of the cell nucleus, a 1:1000 dilution of Hoechst stain (Molecular Probes, Catalogue No. H3570) was added. Each plate was washed in turn with PBS. Subsequently, PBS (100 μl) was added to each well and the plates were analysed using a Cellomics array scan to assess total cell number and number of BrdU positive cells.

Fluorescence dose response data obtained with each compound were analysed and the degree of inhibition of MDA-MB-468 cell growth was expressed as an IC₅₀ value.

Although the pharmacological properties of the compounds of formula (I) vary with structural change as expected, in general, it is believed that activity possessed by compounds of formula (I) may be demonstrated at the following concentrations or doses in one or more of the above tests (a) to (d):
Test (a)(i): IC₅₀ versus mTOR kinase at less than 10 μM, in particular 0.001-0.5 μM for many compounds; for example 2b the IC₅₀ was measured on three occasions, the values were 0.059, 0.005 and 0.023 μM.
Test (b)(i): IC₅₀ versus p110γ Type Ib human PI3K at less than 10 μM, in particular 0.001-0.5 μM for many compounds; and IC₅₀ versus p110α Type Ia human PI3K at less than 10 μM, in particular 0.001-0.5 μM for many compounds; for example 2b the IC50 was measured, the value was 1.044 μM.
Test (c): IC₅₀ versus Serine 473 in Akt at less than 10 μM, in particular 0.1-20 μM for many compounds); for example 2b the IC₅₀ was measured on two occasions, the values were 0.052 and 0.025 μM.
Test (d): IC₅₀ at less than 20 μM.

The following examples were tested in enzyme assay Test (a)(ii):

| | Test (a)(ii) |
|---|---|
| Ex No. | IC₅₀ (μM) |
| 1 | 0.00276 |
| 1a | 0.00279 |
| 2 | 0.0015 |
| 2a | 0.0932 |
| 2b | 0.000169 |
| 2c | 0.00186 |
| 2d | 0.0239 |
| 2e | 0.279 |
| 2f | 0.00433 |
| 2g | 0.000231 |
| 2h | 0.000272 |
| 2i | 0.00207 |
| 2j | 0.012 |
| 2k | 0.00381 |
| 2l | 0.0116 |
| 2m | 0.00589 |
| 2n | 0.00844 |
| 2o | 0.000403 |
| 2p | 0.0027 |
| 2q | 0.000379 |
| 2r | 0.00103 |
| 2s | 0.00155 |
| 2t | 0.000656 |
| 2u | 0.00118 |
| 2v | 0.00866 |
| 2w | 0.0685 |
| 2x | 0.00164 |
| 2y | 0.23 |
| 2z | 0.0181 |
| 2aa | 0.0274 |
| 2ab | 0.0553 |
| 2ac | 0.00641 |
| 2ad | 0.000705 |
| 2ae | 0.00072 |
| 2af | 0.0462 |
| 2ag | 0.0149 |
| 3 | 0.00195 |
| 3a | 0.00445 |
| 3b | 0.00385 |
| 3c | 0.0124 |
| 3d | 0.0135 |
| 3e | 0.00166 |
| 3f | 0.0214 |
| 3g | 0.00126 |
| 3h | 0.0165 |
| 3i | 0.000932 |
| 3j | 0.00422 |
| 3k | 0.00227 |
| 3l | 0.00257 |
| 3m | 0.000497 |
| 3n | 0.0396 |
| 3o | 0.00596 |
| 3p | 0.193 |
| 3q | 0.00839 |
| 3r | 0.0488 |
| 3s | 0.0263 |
| 3t | 0.00479 |
| 3u | 0.000604 |
| 3w | 0.00089 |
| 4 | 0.000699 |
| 4a | 0.00217 |
| 4b | 0.00203 |
| 4c | 0.0169 |
| 4d | 0.01 |
| 4e | 0.000767 |
| 4f | 0.01 |
| 4g | 0.0626 |
| 4h | 0.00146 |
| 4i | 0.0115 |
| 4j | 0.00379 |
| 4k | 0.00905 |
| 4l | 0.00369 |
| 4m | 0.00245 |
| 4n | 0.0392 |
| 5 | 0.000802 |
| 5a | 0.000289 |
| 5b | 0.00052 |
| 5c | 0.00681 |
| 5d | 0.00274 |
| 5e | 0.000864 |
| 5f | 0.00485 |
| 5g | 0.0668 |
| 5h | 0.00234 |
| 5i | 0.00988 |
| 5j | 0.000173 |
| 5k | 0.00333 |
| 5l | 0.000237 |
| 5m | 0.000604 |
| 5n | 0.00828 |
| 6 | 0.001 |
| 6a | 0.00293 |
| 6b | 0.00612 |
| 6c | 0.00321 |
| 6d | 0.000874 |
| 6e | 0.0673 |
| 6f | 0.000794 |
| 6g | 0.00225 |
| 6h | 0.000799 |
| 6i | 0.00462 |
| 6j | 0.00593 |
| 6k | 0.00186 |
| 6l | 0.00119 |
| 6m | 0.000936 |
| 7 | 0.00315 |
| 7a | 0.00231 |
| 7b | 0.0181 |
| 7c | 0.00646 |
| 7d | 0.0038 |
| 7e | 0.00212 |
| 7f | 0.00915 |
| 7g | 0.211 |
| 7h | 0.0116 |
| 7i | 0.0182 |
| 7j | 0.0105 |
| 7k | 0.00482 |
| 7l | 0.00913 |
| 7m | 0.00504 |
| 8 | 0.000959 |
| 8a | 0.00096 |
| 8b | 0.00123 |
| 8c | 0.00185 |
| 8d | 0.00134 |
| 8e | 0.000342 |
| 8f | 0.00882 |
| 8g | 0.0784 |
| 8h | 0.00176 |
| 8i | 0.0198 |
| 8j | 0.000751 |
| 8k | 0.0173 |
| 8l | 0.00813 |
| 8m | 0.00136 |
| 9 | 0.0018 |
| 9a | 0.0129 |
| 9b | 0.0215 |
| 9c | 0.0483 |
| 9d | 0.0151 |
| 9e | 0.00187 |
| 9f | 0.144 |
| 9g | 0.00127 |
| 9h | 0.0057 |
| 9i | 0.0024 |
| 9j | 0.046 |
| 9k | 0.0115 |
| 9l | 0.00395 |
| 9m | 0.00401 |
| 10 | 0.00448 |
| 10a | 0.0395 |
| 10b | 0.0266 |
| 10c | 0.147 |
| 10d | 0.0432 |
| 10e | 0.00691 |
| 10f | 0.174 |
| 10g | 0.00156 |
| 10h | 0.0268 |
| 10i | 0.00591 |
| 10j | 0.11 |
| 10k | 0.0502 |
| 10l | 0.0183 |
| 10m | 0.00497 |
| 11 | 0.00165 |
| 11a | 0.0059 |
| 11b | 0.00333 |
| 11c | 0.0247 |
| 11d | 0.00642 |
| 11e | 0.00197 |
| 11f | 0.00337 |
| 11g | 0.313 |
| 11h | 0.00913 |
| 11i | 0.0294 |
| 11j | 0.0122 |
| 11k | 0.00332 |
| 11l | 0.00356 |
| 11m | 0.00437 |
| 12 | 0.00421 |
| 12a | 0.00167 |
| 12b | 0.00271 |
| 12c | 0.00751 |
| 12d | 0.00155 |
| 12e | 0.00459 |
| 12f | 0.35 |
| 12g | 0.00273 |
| 12h | 0.00392 |
| 12i | 0.000771 |
| 12j | 0.00167 |
| 12k | 0.0043 |
| 12l | 0.00138 |
| 12m | 0.0101 |
| 12n | 0.238 |
| 12o | 0.00509 |
| 12p | 0.0014 |
| 12q | 0.00162 |
| 12r | 0.000991 |
| 12s | 0.117 |
| 12t | 0.0366 |
| 13 | 0.00357 |
| 13a | 0.00341 |
| 13b | 0.0037 |
| 13c | 0.00173 |
| 13d | 0.00351 |
| 13e | 0.0301 |
| 13f | 0.386 |
| 13g | 0.00479 |
| 13h | 0.0106 |
| 13i | 0.00275 |
| 13j | 0.0178 |
| 13k | 0.0179 |
| 13l | 0.0108 |
| 13m | 0.0532 |
| 13n | 0.997 |
| 13o | 0.0287 |
| 13p | 0.00202 |
| 13q | 0.00192 |
| 13r | 0.00198 |
| 13s | 0.00846 |
| 13t | 0.172 |
| 13u | 0.00477 |
| 14 | 0.0465 |
| 14a | 0.0136 |
| 14b | 1.39 |
| 14c | 0.0401 |
| 14d | 0.0143 |
| 14e | 0.11 |
| 14f | 0.189 |
| 14g | 0.0857 |
| 14h | 0.14 |
| 14i | 0.0339 |
| 14j | 0.0129 |
| 14k | 0.275 |
| 14l | 0.0137 |
| 14m | 0.0143 |
| 14n | 0.0363 |
| 14o | 0.0217 |
| 14p | 0.537 |
| 14q | 0.0265 |
| 14r | 0.0103 |
| 14s | 0.122 |
| 14t | 0.197 |
| 14u | 0.123 |
| 14v | 0.0823 |
| 14w | 0.208 |
| 14x | 0.63 |
| 14y | 0.152 |
| 14z | 0.171 |
| 14aa | 0.0186 |
| 15 | 0.00154 |
| 15a | 0.000591 |
| 16 | 0.00142 |
| 16a | 0.0101 |
| 16b | 0.00577 |
| 16c | 0.0016 |
| 16d | 0.00364 |
| 16e | 0.0214 |
| 16f | 0.0212 |
| 16g | 0.0176 |
| 16h | 0.186 |
| 16i | 0.845 |
| 17 | 0.00116 |
| 17a | 0.000942 |
| 17b | 0.000584 |
| 17c | 0.00179 |
| 18 | 0.00135 |
| 18a | 0.00714 |
| 18b | 0.0147 |
| 18c | 0.032 |
| 18d | 0.0151 |
| 18e | 0.000654 |
| 18f | 0.12 |
| 18g | 0.00123 |
| 18h | 0.00664 |
| 18i | 0.00555 |
| 18j | 0.0303 |
| 18k | 0.0113 |
| 18l | 0.0102 |
| 18m | 0.00315 |
| 19 | 0.00131 |
| 19a | 0.00425 |
| 19b | 0.00119 |
| 19c | 0.0214 |
| 19d | 0.00318 |
| 19e | 0.00135 |
| 19f | 0.0804 |
| 19g | 0.000274 |
| 19h | 0.00246 |
| 19i | 0.00152 |
| 19j | 0.0199 |
| 19k | 0.00415 |
| 19l | 0.00152 |
| 19m | 0.000944 |
| 20 | 0.0128 |
| 20a | 0.0163 |
| 20b | 0.874 |
| 20c | 0.00559 |
| 20d | 0.0133 |
| 21 | 0.0253 |
| 21a | 0.0378 |
| 21b | 0.0133 |
| 21c | 0.0234 |
| 22 | 0.503 |
| 23 | 0.00492 |
| 23a | 0.00459 |
| 23b | 0.197 |
| 23c | 0.000831 |
| 23d | 0.00534 |
| 23e | 0.00876 |
| 23f | 0.0199 |
| 23g | 1.21 |
| 23h | 0.00808 |
| 23i | 0.0254 |
| 23j | 0.0314 |
| 23k | 1.14 |
| 23l | 0.00888 |
| 23m | 0.0577 |
| 24 | 0.00517 |
| 25 | 0.00155 |
| 25a | 0.00224 |
| 25b | 0.000817 |
| 25c | 0.00385 |
| 25d | 0.00152 |
| 25e | 0.000277 |
| 25f | 0.0547 |
| 25g | 0.00143 |
| 25h | 0.000993 |
| 25i | 0.001 |
| 25j | 0.00576 |
| 25k | 0.00292 |
| 25l | 0.000956 |
| 25m | 0.00025 |
| 26 | 0.00605 |
| 26a | 0.00672 |
| 26b | 0.0039 |
| 26c | 0.119 |
| 26d | 0.0012 |
| 26e | 0.00395 |
| 26f | 0.00457 |
| 26g | 0.00746 |
| 26h | 0.004 |
| 27 | 0.00111 |
| 27a | 0.00388 |
| 27b | 0.00425 |
| 27c | 0.014 |
| 27d | 0.00307 |
| 27e | 0.000351 |
| 27f | 0.000997 |
| 27g | 0.0213 |
| 27h | 0.000244 |
| 27i | 0.00302 |
| 27j | 0.00761 |
| 27k | 0.00462 |
| 27l | 0.00112 |
| 27m | 0.00144 |
| 28 | 0.00187 |
| 28a | 0.00253 |
| 28b | 0.00149 |
| 28c | 0.00847 |
| 28d | 0.00237 |
| 28e | 0.000434 |
| 28f | 0.0674 |
| 28g | 0.00118 |
| 28h | 0.00093 |
| 28i | 0.00153 |
| 28j | 0.00557 |
| 28k | 0.000954 |
| 28l | 0.000442 |
| 29 | 0.0042 |
| 30 | 0.025 |
| 31 | 0.00452 |
| 31a | 0.00265 |
| 31b | 0.00497 |
| 31c | 0.00131 |
| 31d | 0.00955 |
| 31e | 0.00107 |
| 31f | 0.0016 |
| 31g | 0.00323 |
| 31h | 0.000209 |
| 31i | 0.00326 |
| 31j | 0.00103 |
| 31k | 0.00191 |
| 31l | 0.214 |
| 31m | 0.00591 |
| 32 | 0.00315 |
| 32a | 0.0425 |
| 32b | 0.00829 |
| 32c | 0.00288 |
| 32d | 0.0131 |
| 33 | 0.00929 |
| 33a | 0.0584 |
| 33b | 0.012 |
| 33c | 0.00258 |
| 33d | 0.133 |
| 34 | 0.00405 |
| 34a | 0.0387 |
| 34b | 0.0109 |
| 34c | 0.00345 |
| 34d | 0.0863 |
| 35 | 0.00531 |
| 35a | 0.00552 |
| 35b | 0.000577 |
| 35c | 0.108 |
| 35d | 0.00411 |
| 36a | 0.142 |
| 36b | 0.00179 |
| 36c | 0.114 |
| 36d | 0.00317 |
| 36e | 0.00667 |
| 36f | 0.00278 |
| 36g | 0.00841 |
| 36h | 0.00314 |
| 36i | 0.0518 |
| 36j | 0.018 |
| 36k | 0.0131 |
| 36l | 0.0428 |
| 36m | 0.0347 |
| 36n | 0.0248 |
| 36o | 0.00358 |
| 36p | 0.00161 |
| 36q | 0.0889 |
| 36r | 0.00733 |
| 36s | 0.00475 |
| 36t | 0.194 |
| 36u | 0.00922 |
| 36v | 0.467 |
| 36w | 0.11 |
| 36x | 0.182 |
| 36y | 0.00131 |
| 36z | 0.00208 |
| 36aa | 0.0749 |
| 36ab | 0.011 |
| 36ac | 0.00441 |
| 36ad | 0.0323 |
| 36ae | 0.0151 |
| 36af | 0.00275 |
| 36ag | 0.0013 |
| 36ah | 0.00247 |
| 36ai | 0.00561 |
| 36aj | 0.00255 |
| 36ak | 0.00245 |
| 36al | 0.0161 |
| 36am | 0.0259 |
| 36an | 0.0104 |
| 36ao | 0.014 |
| 36ap | 0.0156 |
| 36aq | 0.0157 |
| 36ar | 0.00945 |
| 36as | 0.00911 |
| 36at | 0.00639 |
| 36au | 0.0121 |
| 36av | 0.00995 |
| 36aw | 0.0164 |
| 36ax | 0.0211 |
| 36ay | 0.00117 |
| 36az | 0.00105 |
| 36ba | 0.00167 |
| 36bb | 0.00513 |
| 36bc | 0.00285 |
| 36bd | 0.00258 |
| 36be | 0.00376 |
| 36bf | 0.00457 |
| 36bg | 0.00189 |
| 36bh | 0.00137 |
| 36bi | 0.00231 |
| 36bj | 0.00106 |
| 36bk | 0.00433 |
| 36bl | 0.00333 |
| 36bm | 0.00193 |
| 36bn | 0.00637 |
| 36bo | 0.0176 |
| 36bp | 0.00198 |
| 36bq | 0.00131 |
| 36br | 0.00188 |
| 36bs | 0.00264 |
| 36bt | 0.00188 |
| 36bu | 0.00118 |
| 36bv | 0.00264 |
| 36bw | 0.00909 |
| 36bx | 0.00269 |
| 36by | 0.012 |
| 36bz | 0.0143 |
| 36ca | 0.31 |
| 36cb | 0.163 |
| 36cc | 0.129 |
| 36cd | 0.147 |
| 36ce | 0.42 |
| 36cf | 0.286 |
| 36cg | 0.136 |
| 36ch | 0.208 |
| 36ci | 0.0386 |
| 36cj | 0.00333 |
| 36ck | 2.62 |
| 36cl | 0.0681 |
| 36cm | 0.00946 |
| 36cn | 0.0858 |
| 36co | 1.62 |
| 36cp | 1.26 |
| 36cq | 0.114 |
| 36cr | 0.0435 |
| 36cs | 1.17 |
| 36ct | 0.00157 |
| 36cu | 0.0686 |
| 36v | 0.259 |
| 36cw | 0.0493 |
| 36cx | 0.284 |
| 36cy | 0.0478 |
| 36cz | 0.0384 |
| 36da | 0.0331 |
| 36db | 0.0209 |
| 36dc | 0.00989 |
| 36dd | 0.00291 |
| 36de | 0.00389 |
| 36df | 0.00712 |
| 36dg | 0.0164 |
| 36dh | 0.0034 |
| 36di | 0.00923 |
| 36dj | 0.00891 |
| 36dk | 0.00905 |
| 36dl | 0.00399 |
| 36dm | 0.0298 |
| 36dn | 0.0138 |
| 36do | 0.0277 |
| 36dp | 0.0106 |
| 36dq | 0.0245 |
| 36dr | 0.294 |
| 36ds | 0.000973 |
| 36dt | 0.00037 |
| 36du | 0.0152 |
| 36dv | 0.0043 |
| 36dw | 0.00624 |
| 36dx | 0.00262 |
| 36dy | 0.00636 |
| 36dz | 0.00277 |
| 36ea | 0.00884 |
| 36eb | 0.00311 |
| 36ec | 0.00321 |
| 36ed | 0.00704 |
| 36ee | 0.00958 |
| 36ef | 0.00841 |
| 36eg | 0.00877 |
| 37a | 0.026 |
| 37b | 0.0124 |
| 37c | 0.0033 |
| 37d | 0.00439 |
| 37e | 0.0049 |
| 37f | 0.12 |
| 37g | 0.0179 |
| 37h | 0.0198 |
| 37i | 0.00379 |
| 37j | 0.0309 |
| 37k | 0.0161 |
| 37l | 0.032 |
| 37m | 0.0506 |
| 37n | 0.0393 |
| 37o | 0.033 |
| 37p | 0.00534 |
| 37q | 0.00294 |
| 37r | 0.00162 |
| 37s | 0.00756 |
| 37t | 0.00889 |
| 37u | 0.000395 |
| 37v | 0.00549 |
| 37w | 0.00137 |
| 37x | 0.00944 |
| 37y | 0.00686 |
| 37z | 0.00481 |
| 37aa | 0.00567 |
| 37ab | 0.00488 |
| 37ac | 0.0438 |
| 37ad | 0.00263 |
| 37ae | 0.00952 |
| 37af | 0.0233 |
| 37ag | 0.0134 |
| 38 | 0.00131 |
| 38a | 0.000918 |
| 39 | 0.000351 |
| 39a | 0.000502 |
| 40 | 0.00436 |
| 40a | 0.00221 |
| 41 | 0.0308 |
| 42 | 0.000705 |
| 42a | 0.00261 |
| 42b | 0.00535 |
| 42c | 0.0036 |
| 42d | 0.000607 |
| 42e | 0.00257 |
| 42f | 0.00264 |
| 42g | 0.00401 |
| 42h | 0.00412 |
| 43 | 0.00539 |
| 43a | 0.00479 |
| 43b | 0.00751 |
| 43c | 0.0288 |
| 43d | 0.011 |
| 43e | 0.188 |
| 43f | 0.0392 |
| 43g | 0.029 |
| 43h | 0.133 |
| 43i | 0.102 |
| 43j | 0.012 |
| 43k | 0.0257 |
| 43l | 0.0236 |
| 43m | 0.00531 |
| 43n | 0.0119 |
| 43o | 0.0157 |
| 43p | 0.0555 |
| 43q | 0.028 |
| 43r | 0.0577 |
| 43s | 0.00629 |
| 43t | 0.00545 |
| 44 | 0.0234 |
| 45 | 0.00717 |
| 46 | 0.00456 |
| 46a | 0.0126 |
| 47 | 0.0155 |
| 47a | 0.0054 |
| 47b | 0.0149 |
| 48 | 0.00072 |
| 48a | 0.00116 |
| 49 | 0.00187 |
| 49a | 0.00267 |
| 50 | 0.0104 |
| 50a | 0.00794 |
| 51 | 0.00332 |
| 52 | 0.0184 |
| 52a | 0.0103 |
| 52b | 0.0307 |
| 52c | 0.0296 |
| 52d | 0.00588 |
| 53 | 0.00218 |
| 53a | 0.00229 |
| 53b | 0.000669 |
| 53c | 0.000688 |
| 53d | 0.0031 |
| 53e | 0.00198 |
| 53f | 0.00634 |
| 54 | 0.00217 |
| 54a | 0.00918 |
| 54b | 0.00317 |
| 54c | 0.00453 |
| 54d | 0.00882 |
| 54e | 0.00762 |
| 54f | 0.0107 |
| 55 | 0.00296 |
| 55a | 0.0213 |
| 55b | 0.0161 |
| 55c | 0.0189 |
| 55d | 0.0351 |
| 55e | 0.0204 |
| 55f | 0.0156 |
| 56 | 0.00231 |
| 56a | 0.00226 |
| 56b | 0.00165 |
| 56c | 0.00623 |
| 56d | 0.0043 |
| 57 | 0.0119 |
| 57a | 0.0104 |
| 57b | 0.00457 |
| 57c | 0.00695 |
| 57d | 0.01 |
| 57e | 0.00728 |
| 57f | 0.0106 |
| 57g | 0.0585 |
| 58 | 0.00115 |
| 58a | 0.00339 |
| 58b | 0.002 |
| 58c | 0.00327 |
| 58d | 0.00293 |
| 58e | 0.00256 |
| 58f | 0.00267 |
| 58g | 0.0102 |
| 59 | 0.00882 |
| 59a | 0.0222 |
| 59b | 0.00993 |
| 59c | 0.0226 |
| 59d | 0.032 |
| 59e | 0.293 |
| 59f | 0.0491 |
| 59g | 0.101 |
| 60 | 0.012 |
| 60a | 0.0191 |
| 60b | 0.00862 |
| 60c | 0.00695 |
| 60d | 0.0183 |
| 60e | 0.0116 |
| 60f | 0.0173 |
| 61 | 0.0269 |
| 61a | 0.0326 |
| 61b | 0.0136 |
| 61c | 0.0211 |
| 61d | 0.0438 |
| 61e | 0.0237 |
| 61f | 0.0328 |
| 62 | 0.00205 |
| 62a | 0.0393 |
| 62b | 0.00546 |
| 62c | 0.0375 |
| 62d | 0.022 |
| 63 | 0.0725 |
| 64 | 0.00785 |
| 64a | 0.0052 |
| 64b | 0.00678 |
| 64c | 0.00215 |
| 64d | 0.0067 |
| 64e | 0.00537 |
| 64f | 0.00521 |
| 64g | 0.0342 |
| 64h | 0.00148 |
| 64i | 0.00135 |
| 64j | 0.0012 |
| 64k | 0.00144 |
| 64l | 0.00111 |
| 64m | 0.0408 |
| 64n | 0.0161 |
| 64o | 0.00345 |
| 64p | 0.00398 |
| 64q | 0.00467 |
| 64r | 0.00418 |
| 64s | 0.00296 |
| 64t | 0.0114 |
| 64u | 0.0139 |
| 64v | 0.146 |
| 64w | 0.0161 |
| 64x | 0.00761 |
| 64y | 0.0125 |
| 64z | 0.00866 |
| 64aa | 0.00388 |
| 64ab | 0.0094 |
| 64ac | 0.00529 |
| 64ad | 0.00298 |
| 64ae | 0.00213 |
| 64af | 0.00339 |
| 64ag | 0.00281 |
| 64ah | 0.00684 |
| 64ai | 0.00567 |
| 64aj | 0.00778 |
| 64ak | 0.00161 |
| 64al | 0.00558 |
| 65 | 0.03 |
| 65a | 0.00688 |
| 65b | 0.00342 |
| 65c | 0.0151 |
| 65d | 0.00448 |
| 65e | 0.0114 |
| 66 | 0.00303 |
| 66a | 0.00415 |
| 66b | 0.0246 |
| 67 | 0.001 |
| 68 | 0.00141 |
| 68a | 0.00174 |
| 69 | 0.00788 |
| 70 | 0.002 |
| 71 | 0.0182 |
| 72 | 0.00432 |
| 73 | 0.00744 |
| 74 | 0.00237 |
| 74a | 0.00398 |
| 74b | 0.00244 |
| 74c | 0.00314 |
| 74d | 0.00279 |
| 74e | 0.00506 |
| 74f | 0.00399 |
| 74g | 0.00433 |
| 74h | 0.00321 |
| 74i | 0.00925 |
| 75 | 0.029 |
| 75a | 0.0435 |
| 75b | 0.0381 |
| 75c | 0.0339 |
| 75d | 0.0399 |
| 75e | 0.037 |
| 75f | 0.0608 |
| 76 | 0.00687 |
| 76a | 0.00407 |
| 76b | 0.00719 |
| 76c | 0.00319 |
| 76d | 0.00924 |
| 76e | 0.00566 |
| 76f | 0.00678 |
| 77 | 0.00821 |
| 77a | 0.00524 |
| 77b | 0.0086 |
| 77c | 0.00437 |
| 77d | 0.0101 |
| 77e | 0.00517 |
| 77f | 0.00919 |
| 78 | 0.0418 |
| 78a | 0.0153 |
| 78b | 0.0333 |
| 78c | 0.0209 |
| 78d | 0.0175 |
| 78e | 0.0239 |
| 78f | 0.0291 |
| 79 | 0.0186 |
| 79a | 0.0222 |
| 79b | 0.0109 |
| 79c | 0.0299 |
| 79d | 0.0161 |
| 79e | 0.0259 |
| 79f | 0.0398 |
| 80 | 0.0197 |
| 80a | 0.0146 |
| 80b | 0.0103 |
| 80c | 0.00921 |
| 80d | 0.00951 |
| 80e | 0.0143 |
| 81 | 0.0137 |
| 82 | 0.00939 |
| 82a | 0.0265 |
| 82b | 0.00757 |
| 82c | 0.0185 |
| 82d | 0.0243 |
| 82e | 0.0208 |
| 82f | 0.0273 |
| 83 | 0.00346 |
| 83a | 0.00483 |
| 83b | 0.0114 |
| 83c | 0.0124 |
| 83d | 0.0472 |
| 84 | 0.00956 |
| 84a | 0.00909 |
| 84b | 0.00385 |
| 84c | 0.00829 |
| 84d | 0.00959 |
| 84e | 0.00818 |
| 84f | 0.0134 |
| 85 | 0.00477 |
| 85a | 0.00313 |
| 85b | 0.00526 |
| 85c | 0.00509 |
| 85d | 0.00323 |
| 85e | 0.0183 |
| 85f | 0.0202 |
| 85g | 0.0259 |
| 85h | 0.0426 |
| 85i | 0.0153 |
| 85j | 0.0124 |
| 86 | 0.00247 |
| 86a | 0.00522 |
| 86b | 0.00725 |
| 86c | 0.00247 |
| 86d | 0.00382 |
| 86e | 0.00223 |
| 86f | 0.00345 |
| 87 | 0.00487 |
| 87a | 0.0887 |
| 87b | 0.0177 |
| 87c | 0.00775 |
| 87d | 0.0177 |
| 87e | 0.0103 |
| 87f | 0.00907 |
| 88 | 0.0321 |
| 88a | 0.00448 |
| 88b | 0.0567 |
| 88c | 0.047 |
| 88d | 0.004 |
| 88e | 0.131 |
| 89 | 0.485 |
| 90 | 0.0189 |
| 91 | 0.00498 |
| 91a | 0.00198 |
| 91b | 0.00521 |
| 91c | 0.00368 |
| 91d | 0.00356 |
| 92 | 0.00793 |
| 92a | 0.00551 |
| 92b | 0.00746 |
| 92c | 0.0078 |
| 92d | 0.00807 |
| 92e | 0.00599 |
| 92f | 0.00625 |
| 93 | 0.00505 |
| 93a | 0.00303 |
| 93b | 0.0023 |
| 93c | 0.0029 |
| 93d | 0.000818 |
| 93e | 0.00541 |
| 94a | 0.0136 |
| 94b | 0.0122 |
| 94c | 0.0118 |
| 94d | 0.011 |
| 94e | 0.0135 |
| 94f | 0.0151 |
| 94g | 0.0127 |
| 94h | 0.0116 |
| 94i | 0.0121 |
| 94j | 0.00605 |
| 94k | 0.0106 |
| 95a | 0.0139 |
| 95b | 0.019 |
| 95c | 0.00706 |
| 95d | 0.0126 |
| 95e | 0.016 |
| 95f | 0.0109 |
| 95g | 0.0164 |
| 95h | 0.013 |
| 95i | 0.00479 |
| 95j | 0.0131 |
| 95k | 0.0272 |
| 95l | 0.00109 |
| 95m | 0.00202 |
| 95n | 0.000668 |
| 95o | 0.00473 |
| 95p | 0.00543 |
| 95q | 0.00168 |
| 95r | 0.00631 |
| 96a | 0.0177 |
| 96b | 0.0268 |
| 96c | 0.0272 |
| 96d | 0.0209 |
| 96e | 0.00862 |
| 97 | 0.0882 |
| 98a | 0.011 |
| 98b | 0.00413 |
| 98c | 0.0267 |
| 98d | 0.00441 |
| 98e | 0.00335 |
| 98f | 0.00229 |
| 98g | 0.0127 |
| 98h | 0.00362 |
| 98i | 0.0285 |
| 98j | 0.00374 |
| 98k | 0.00246 |
| 98l | 0.00789 |
| 99a | 0.0438 |
| 99b | 0.0316 |
| 99c | 0.239 |
| 99d | 0.219 |
| 99e | 0.0317 |
| 99f | 0.00589 |
| 99g | 0.0152 |
| 99h | 0.0782 |
| 99i | 0.0594 |
| 99j | 0.0124 |

Compounds may be further selected on the basis of further biological or physical properties which may be measured by techniques known in the art and which may be used in the assessment or selection of compounds for therapeutic or prophylactic application.

The compounds of the present invention are advantageous in that they possess pharmacological activity. In particular, the compounds of the present invention modulate (in particular, inhibit) mTOR kinase and/or phosphatidylinositol-3-kinase (PI3K) enzymes, such as the Class Ia PI3K enzymes (e.g. PI3Kalpha, PI3Kbeta and PI3Kdelta) and the Class Ib PI3K enzyme (PI3Kgamma). More particularly compounds of the present invention modulate (in particular, inhibit) mTOR kinase. More particularly compounds of the present invention modulate (in particular, inhibit) one or more PI3K enzyme. The inhibitory properties of compounds of formula (I) may be demonstrated using the test procedures set out herein and in the experimental section. Accordingly, the compounds of formula (I) may be used in the treatment (therapeutic or prophylactic) of conditions/diseases in human and non-human animals which are mediated by mTOR kinase and/or one or more PI3K enzyme(s), and in particular by mTOR kinase.

The invention also provides a pharmaceutical composition comprising a compound of formula (I), or a pharmaceutically acceptable salt thereof, as defined herein in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of formula I will naturally vary according to the nature and severity of the disease state, the age and sex of the animal or patient and the route of administration, according to well known principles of medicine.

In using a compound of formula (I) for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Typically, unit dosage forms will contain about 10 mg to 0.5 g of a compound of this invention.

As stated herein, it is known that mTOR kinase and the PI3K enzymes have roles in tumourigenesis as well as numerous other diseases. We have found that the compounds of formula (I) possess potent anti-tumour activity which it is believed is obtained by way of inhibition of mTOR kinase and/or one or more of the PI3K enzymes.

Accordingly, the compounds of the present invention are of value as anti-tumour agents. Particularly, the compounds of the present invention are of value as anti-proliferative, apoptotic and/or anti-invasive agents in the containment and/or treatment of solid and/or liquid tumour disease. Particularly, the compounds of the present invention are expected to be useful in the prevention or treatment of those tumours which are sensitive to inhibition of mTOR and/or one or more of the PI3K enzymes such as the Class Ia PI3K enzymes and the Class Ib PI3K enzyme. Further, the compounds of the present invention are expected to be useful in the prevention or treatment of those tumours which are mediated alone or in part by mTOR and/or one or more of the PI3K enzymes such as the Class Ia PI3K enzymes and the Class Ib PI3K enzyme. The compounds may thus be used to produce an mTOR enzyme inhibitory effect in a warm-blooded animal in need of such treatment. Certain compounds may be used to produce an PI3K enzyme inhibitory effect in a warm-blooded animal in need of such treatment.

As stated herein, inhibitors of mTOR kinase and/or one or more PI3K enzymes should be of therapeutic value for the treatment of proliferative disease such as cancer and in particular solid tumours such as carcinoma and sarcomas and the leukaemias and lymphoid malignancies and in particular for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias [including acute lymphoctic leukaemia (ALL) and chronic myelogenous leukaemia (CML)], multiple myeloma and lymphomas.

Anti-cancer effects which are accordingly useful in the treatment of cancer in a patient include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. Anti-cancer effects include prophylactic treatment as well as treatment of existing disease.

A mTOR kinase inhibitor, or a pharmaceutically acceptable salt thereof, may also be useful for the treatment patients with cancers, including, but not limited to, haematologic malignancies such as leukaemia, multiple myeloma, lymphomas such as Hodgkin's disease, non-Hodgkin's lymphomas (including mantle cell lymphoma), and myelodysplastic syndromes, and also solid tumours and their metastases such as breast cancer, lung cancer (non-small cell lung cancer (NSCL), small cell lung cancer (SCLC), squamous cell carcinoma), endometrial cancer, tumours of the central nervous system such as gliomas, dysembryoplastic neuroepithelial tumour, glioblastoma multiforme, mixed gliomas, medulloblastoma, retinoblastoma, neuroblastoma, germinoma and teratoma, cancers of the gastrointestinal tract such as gastric cancer, oesophagal cancer, hepatocellular (liver) carcinoma, cholangiocarcinomas, colon and rectal carcinomas, cancers of the small intestine, pancreatic cancers, cancers of the skin such as melanomas (in particular metastatic melanoma), thyroid cancers, cancers of the head and neck and cancers of the salivary glands, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma, Ewing's sarcoma, gastrointestinal stromal tumour (GIST), Kaposi's sarcoma, and paediatric cancers such as rhabdomyosarcomas and neuroblastomas.

The compounds of the present invention and the methods of treatment comprising the administering or use of a mTOR kinase inhibitor, or a pharmaceutically acceptable salt thereof, are expected to be particularly useful for the treatment of patients with lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment of patients with acute myeloid leukaemia.

According to a further aspect of the invention there is provided a compound of formula (I), or a pharmaceutically acceptable salt thereof, as defined herein for use as a medicament in a warm-blooded animal such as man.

According to a further aspect of the invention, there is provided a compound of formula (I), or a pharmaceutically acceptable salt thereof, as defined herein for use in the production of an anti-proliferative effect in a warm-blooded animal such as man.

According to a further aspect of the invention, there is provided a compound of formula (I), or a pharmaceutically acceptable salt thereof, as defined herein for use in the production of an apoptotic effect in a warm-blooded animal such as man.

According to a further feature of the invention there is provided a compound of formula (I), or a pharmaceutically acceptable salt thereof, as defined herein for use in a warm-blooded animal such as man as an anti-invasive agent in the containment and/or treatment of proliferative disease such as cancer.

According to a further aspect of the invention, there is provided the use of a compound of formula (I), or a pharmaceutically acceptable salt thereof, as defined herein for the production of an anti-proliferative effect in a warm-blooded animal such as man.

According to a further feature of this aspect of the invention there is provided the use of a compound of formula (I), or a pharmaceutically acceptable salt thereof, as defined herein in the manufacture of a medicament for use in the production of an anti-proliferative effect in a warm-blooded animal such as man.

According to a further aspect of the invention, there is provided the use of a compound of formula (I), or a pharmaceutically acceptable salt thereof, as defined herein for the production of an apoptotic effect in a warm-blooded animal such as man.

According to a further feature of this aspect of the invention there is provided the use of a compound of formula (I), or a pharmaceutically acceptable salt thereof, as defined herein in the manufacture of a medicament for use in the production of an apoptotic effect in a warm-blooded animal such as man.

According to a further feature of the invention there is provided the use of a compound of formula (I), or a pharmaceutically acceptable salt thereof, as defined herein in the manufacture of a medicament for use in a warm-blooded animal such as man as an anti-invasive agent in the containment and/or treatment of proliferative disease such as cancer.

According to a further feature of this aspect of the invention there is provided a method for producing an anti-proliferative effect in a warm-blooded animal, such as man, in need of such treatment which comprises administering to said animal an effective amount of a compound of formula (I), or a pharmaceutically acceptable salt thereof, as defined herein.

According to a further feature of this aspect of the invention there is provided a method for producing an anti-invasive effect by the containment and/or treatment of solid tumour disease in a warm-blooded animal, such as man, in need of such treatment which comprises administering to said animal an effective amount of a compound of formula (I), or a pharmaceutically acceptable salt thereof, as defined herein.

According to a further aspect of the invention there is provided the use of a compound of formula (I), or a pharmaceutically acceptable salt thereof, as defined herein in the manufacture of a medicament for use in the prevention or treatment of proliferative disease such as cancer in a warm-blooded animal such as man.

According to a further feature of this aspect of the invention there is provided a method for the prevention or treatment of proliferative disease such as cancer in a warm-blooded animal, such as man, in need of such treatment which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in the prevention or treatment of those tumours which are sensitive to inhibition of mTOR kinase and/or one or more PI3K enzymes (such as the Class la enzymes and/or the Class Ib PI3K enzyme) that are involved in the signal transduction steps which lead to the proliferation, survival, invasiveness and migratory ability of tumour cells.

According to a further feature of this aspect of the invention there is provided the use of a compound of formula (I), or a pharmaceutically acceptable salt thereof, as defined herein in the manufacture of a medicament for use in the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells.

According to a further feature of this aspect of the invention there is provided a method for the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in providing a mTOR kinase inhibitory effect and/or a PI3K enzyme inhibitory effect (such as a Class Ia PI3K enzyme or Class Ib PI3K enzyme inhibitory effect).

According to a further feature of this aspect of the invention there is provided the use of a compound of formula (I), or a pharmaceutically acceptable salt thereof, as defined herein in the manufacture of a medicament for use in providing a mTOR kinase inhibitory effect and/or a PI3K enzyme inhibitory effect (such as a Class Ia PI3K enzyme or Class Ib PI3K enzyme inhibitory effect).

According to a further aspect of the invention there is also provided a method for providing a mTOR kinase inhibitory effect and/or a PI3K enzyme inhibitory effect (such as a Class Ia PI3K enzyme or Class Ib PI3K enzyme inhibitory effect) which comprises administering an effective amount of a compound of formula I, or a pharmaceutically acceptable salt thereof, as defined herein.

According to a further feature of the invention there is provided a compound of formula I, or a pharmaceutically acceptable salt thereof, as defined herein for use in the treatment of cancer, inflammatory diseases, obstructive airways diseases, immune diseases or cardiovascular diseases.

According to a further feature of the invention there is provided a compound of formula I, or a pharmaceutically acceptable salt thereof, as defined herein for use in the treatment of solid tumours such as carcinoma and sarcomas and the leukaemias and lymphoid malignancies.

According to a further feature of the invention there is provided a compound of formula I, or a pharmaceutically acceptable salt thereof, as defined herein for use in the treatment of cancer of the breast, colorectum, lung (including small cell lung cancer, non-small cell lung cancer and bronchioalveolar cancer) and prostate.

According to a further feature of the invention there is provided a compound of formula (I), or a pharmaceutically acceptable salt thereof, as defined herein for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided a compound of formula (I), or a pharmaceutically acceptable salt thereof, as defined herein for use in the treatment of cancer of the bile duct, bone, bladder, head and neck, kidney, liver, gastrointestinal tissue, oesophagus, ovary, endometrium, pancreas, skin, testes, thyroid, uterus, cervix and vulva, and of leukaemias (including ALL and CML), multiple myeloma and lymphomas.

According to a further feature of the invention there is provided a compound of formula (I), or a pharmaceutically acceptable salt thereof, as defined herein for use in the treatment of lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment acute myeloid leukaemia.

According to a further feature of the invention there is provided the use of a compound of formula (I), or a pharmaceutically acceptable salt thereof, as defined herein in the manufacture of a medicament for use in the treatment of cancer, inflammatory diseases, obstructive airways diseases, immune diseases or cardiovascular diseases.

According to a further feature of the invention there is provided the use of a compound of formula (I), or a pharmaceutically acceptable salt thereof, as defined herein in the manufacture of a medicament for use in the treatment of of solid tumours such as carcinoma and sarcomas and the leukaemias and lymphoid malignancies.

According to a further feature of the invention there is provided the use of a compound of formula (I), or a pharmaceutically acceptable salt thereof, as defined herein in the manufacture of a medicament for use in the treatment of cancer of the breast, colorectum, lung (including small cell lung cancer, non-small cell lung cancer and bronchioalveolar cancer) and prostate.

According to a further feature of the invention there is provided the use of a compound of formula (I), or a pharmaceutically acceptable salt thereof, as defined herein in the manufacture of a medicament for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided the use of a compound of formula (I), or a pharmaceutically acceptable salt thereof, as defined herein in the manufacture of a medicament for use in the treatment of lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment acute myeloid leukaemia.

According to a further feature of the invention there is provided a method for treating cancer, inflammatory diseases, obstructive airways diseases, immune diseases or cardiovascular diseases in a warm blooded animal such as man that is in need of such treatment which comprises administering an effective amount of a compound of formula (I), or a pharmaceutically acceptable salt thereof, as defined herein.

According to a further feature of the invention there is provided a method for treating solid tumours such as carcinoma and sarcomas and the leukaemias and lymphoid malignancies in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the breast, colorectum, lung (including small cell lung cancer, non-small cell lung cancer and bronchioalveolar cancer) and prostate in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas in a warm blooded animal such as man that is in need of such treatment which comprises administering an effective amount of a compound of formula (I), or a pharmaceutically acceptable salt thereof, as defined herein.

According to a further feature of the invention there is provided a method for treating lung cancer, prostate cancer, melanoma, ovarian cancer, breast cancer, endometrial cancer, kidney cancer, gastric cancer, sarcomas, head and neck cancers, tumours of the central nervous system and their metastases, and acute myeloid leukaemia in a warm blooded animal such as man that is in need of such treatment which comprises administering an effective amount of a compound of formula (I), or a pharmaceutically acceptable salt thereof, as defined herein.

As stated herein, the in vivo effects of a compound of formula (I) may be exerted in part by one or more metabolites that are formed within the human or animal body after administration of a compound of formula (I).

The invention further relates to combination therapies wherein a compound of formula (I), or a pharmaceutically acceptable salt thereof, or a pharmaceutical composition or formulation comprising a compound of formula (I) is administered concurrently or sequentially or as a combined preparation with another treatment of use in the control of oncology disease.

In particular, the treatment defined herein may be applied as a sole therapy or may involve, in addition to the compounds of the invention, conventional surgery or radiotherapy or chemotherapy. Accordingly, the compounds of the invention can also be used in combination with existing therapeutic agents for the treatment of cancer.

Suitable agents to be used in combination include:
(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as famesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense agent;
(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

Unless stated otherwise, starting materials were commercially available. All solvents and commercial reagents were of laboratory grade and were used as received.

In the examples ¹H NMR spectra were recorded on a Bruker DPX 300 (300 MHz), Bruker DRX 400 (400 MHz) instrument or a Bruker DRX 500 (500 MHz) instrument. The central peaks of chloroform-d (δ_{H} 7.27 ppm), dimethylsulfoxide-d₆ (δ_{H} 2.50 ppm) or acetone-d₆ (δ_{H} 2.05 ppm) were used as internal references. The following abbreviations have been used:
s, singlet; d, doublet;
t, triplet; q, quartet; m, multiplet; br, broad.

Column chromatography was carried out using silica gel (0.04-0.063 mm, Merck). In general, a Kromasil KR-100-5-C18 reversed-phase column (250×20 mm, Akzo Nobel) was used for preparative HPLC with mixtures of acetonitrile and water [containing 0.1% trifluoroacetic acid (TFA)] used as the eluent at a flow rate of 10 mL/min.

The following methods were used for liquid chromatography (LC)/mass spectral (MS) analysis:

| | |
|---|---|
| HPLC: | Agilent 1100 or Waters Alliance HT (2790 & 2795) |
| Mass Spectrometer: | Waters ZQ ESCi |

HPLC Column

The standard HPLC column used is the Phemonenex Gemini C18 5 μm, 50×2 mm.

Acidic HPLC Methods

| | | |
|---|---|---|
| The mobile phases | Mobile phase A: | Water |
| used are: | Mobile Phase B: | Acetonitrile |
| | Mobile Phase C: | 1% Formic Acid in 50:50 |
| | | Water:MeCN (v/v) |

Each method is followed by a rapid equilibration using a 5 mL flow rate for 0.45 min.

Four Generic HPLC Methods are Available:

5 Minute Monitor Acidic method

| | Mobile | | | | Flow |
|---|---|---|---|---|---|
| | Phase | Mobile Phase | Mobile Phase | | Rate/ |
| Time/min | A: | B: | C: | Curve | mL/min |
| 0.00 | 95 | 0 | 5 | 1 | 1.1 |
| 4 | 0 | 95 | 5 | 6 | 1.1 |
| 4.5 | 0 | 95 | 5 | 6 | 1.1 |

Early Acidic method for early eluting compounds

| | Mobile | | | | Flow |
|---|---|---|---|---|---|
| | Phase | Mobile Phase | Mobile Phase | | Rate/ |
| Time/min | A: | B: | C: | Curve | mL/min |
| 0.00 | 95 | 0 | 5 | 1 | 1.1 |
| 4 | 57.5 | 37.5 | 5 | 6 | 1.1 |
| 4.5 | 57.5 | 37.5 | 5 | 6 | 1.1 |

Mid Acidic method for middle eluting compounds

| | Mobile | | | | Flow |
|---|---|---|---|---|---|
| | Phase | Mobile Phase | Mobile Phase | | Rate/ |
| Time/min | A: | B: | C: | Curve | mL/min |
| 0.00 | 95 | 0 | 5 | 1 | 1.1 |
| 0.01 | 67.5 | 27.5 | 5 | 6 | 1.1 |
| 4.5 | 27.5 | 67.5 | 5 | 6 | 1.1 |

Late Acidic method for late eluting compounds

| | Mobile | | | | Flow |
|---|---|---|---|---|---|
| | Phase | Mobile Phase | Mobile Phase | | Rate/ |
| Time/min | A: | B: | C: | Curve | mL/min |
| 0.00 | 95 | 0 | 5 | 1 | 1.1 |
| 0.01 | 27.5 | 67.5 | 5 | 6 | 1.1 |
| 4.5 | 5 | 95 | 5 | 6 | 1.1 |

Basic HPLC Methods

In some instances the standard acidic methods may be unsuitable for either the compound ionisation or the chromatography separation required. In such cases four comparable Basic HPLC methods are available.

| | | |
|---|---|---|
| The mobile | Mobile phase A: | Water |
| phases used are: | Mobile Phase B: | Acetonitrile |
| | Mobile Phase D: | 0.1% 880 Ammonia in acetonitrile |

Each method is followed by a rapid equilibration using a 5 mL flow rate for 0.45 min.

Minute Monitor Basic method

| | Mobile | | | | Flow |
|---|---|---|---|---|---|
| | Phase | Mobile Phase | Mobile Phase | | Rate/ |
| Time/min | A: | B: | D: | Curve | mL/min |
| 0.00 | 95 | 0 | 5 | 1 | 1.1 |
| 4 | 0 | 95 | 5 | 6 | 1.1 |
| 4.5 | 0 | 95 | 5 | 6 | 1.1 |

Early Basic method for early eluting compounds

| | Mobile | | | | Flow |
|---|---|---|---|---|---|
| | Phase | Mobile Phase | Mobile Phase | | Rate/ |
| Time/min | A: | B: | D: | Curve | mL/min |
| 0.00 | 95 | 0 | 5 | 1 | 1.1 |
| 4 | 57.5 | 37.5 | 5 | 6 | 1.1 |
| 4.5 | 57.5 | 37.5 | 5 | 6 | 1.1 |

Mid Basic method for middle eluting compounds

| | Mobile | | | | Flow |
|---|---|---|---|---|---|
| | Phase | Mobile Phase | Mobile Phase | | Rate/ |
| Time/min | A: | B: | D: | Curve | mL/min |
| 0.00 | 95 | 0 | 5 | 1 | 1.1 |
| 0.01 | 67.5 | 27.5 | 5 | 6 | 1.1 |
| 4.5 | 27.5 | 67.5 | 5 | 6 | 1.1 |

Late Basic method for late eluting compounds

| | Mobile | | | | Flow |
|---|---|---|---|---|---|
| | Phase | Mobile Phase | Mobile Phase | | Rate/ |
| Time/min | A: | B: | C: | Curve | mL/min |
| 0.00 | 95 | 0 | 5 | 1 | 1.1 |
| 0.01 | 27.5 | 67.5 | 5 | 6 | 1.1 |
| 4.5 | 5 | 95 | 5 | 6 | 1.1 |

The following method was used for liquid chromatography (LC)/mass spectral (MS) analysis: Instrument: Agilent 1100; Column: Waters ‘Symmetry’ 2.1×30 mm; Mass Spectral analysis using chemical ionisation (APCI); Flow rate: 0.7 mL/min; Absorption Wavelength: 254 nm; Solvent A: water+0.1% TFA; Solvent B: acetonitrile+0.1% TFA; Solvent Gradient: 15-95% Solvent B for 2.7 minutes followed by 95% Solvent B for 0.3 minutes.

The following methods were used for LC analysis:

Method A: Instrument: Agilent 1100; Column: Kromasil C18 reversed-phase silica, 100×3 mm, 5 μm particle size; Solvent A: 0.1% TFA/water, Solvent B: 0.08% TFA/acetonitrile; Flow Rate: 1 mL/min; Solvent Gradient: 10-100% Solvent B for 20 minutes followed by 100% Solvent B for 1 minute; Absorption Wavelengths: 220, 254 and 280 nm. In general, the retention time of the product was noted.

Method B: Instrument: Agilent 1100; Column: Waters ‘Xterra’ C8 reversed-phase silica, 100×3 mm, 5 μm particle size; Solvent A: 0.015M ammonia in water, Solvent B: acetonitrile; Flow Rate: 1 ml/min, Solvent Gradient: 10-100% Solvent B for 20 minutes followed by 100% Solvent B for 1 minute; Absorption Wavelength: 220, 254 and 280 nm. In general, the retention time of the product was noted.

The following abbreviations are used herein or within the following illustrative examples:
HPLC High Performance Liquid Chromatography
HBTU O-(benzotriazol-1-yl)-N,N,N′,N′-tetramethyluronium hexafluorophosphate;
HATU O-(7-azabenzotriazol-1-yl)-N,N,N′,N′-tetramethyluronium hexafluorophosphate;
HOBT 1-hydroxybenzotriazole;
HOAT 1-hydroxy-7-azabenzotriazole;
NMP N-methylpyrrolidin-2-one;
DMSO dimethylsulfoxide;
DMF N,N-dimethylformamide;
DMA N,N-dimethylacetamide;
THF tetrahydrofuran;
DME 1,2-dimethoxyethane;
DCCI dicyclohexylcarbodiimide;
MeOH methanol;
MeCN acetonitrile;
DCM dichloromethane;
DIPEA N,N-diisopropylethylamine;
DBU 1,8-diazabicyclo[5.4.0]undec-7-ene;
RT room temperature (approximately 17 to 25° C.);
tR retention time;
m/z mass/charge ratio.

The chemical names were generated by software which used the Lexichem Toolkit (v. 1.60) from OpenEye Scientific Software (www.eyesopen.com) to generate IUPAC conforming names.

### EXAMPLE 1

### 3-Ethyl-1-[4-[4-[(3S)-3-methylmorpholin-4-yl]-6-(1-methylsulfonylcyclopropyl)pyrimidin-2-yl]phenyl]urea

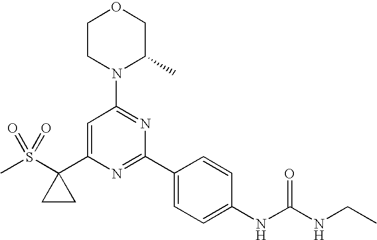

To a solution of 2-chloro-4-[(3S)-3-methylmorpholin-4-yl]-6-(1-methylsulfonylcyclopropyl)pyrimidine (200 mg, 0.60 mmol) in ethanol was added toluene (1.00 mL), water (1.00 mL), [4-(3-ethylureido)phenyl]boronic acid, pinacol ester (262 mg, 0.90 mmol), tri-potassium orthophosphate (448 mg, 2.11 mmol) and palladiumbis(tri-tert-butylphosphine) (18.55 mg, 0.04 mmol). The reaction was degassed then purged with nitrogen and heated at 80° C. for 2 hours. The reaction mixture was diluted with ethyl acetate (10 mL) and washed with water (5 mL). The organic layer was dried (MgSO₄), filtered and evaporated to afford crude product which was purified by flash silica chromatography, elution gradient 0 to 10% (3.5M ammonia in methanol) in DCM, to give the desired material as a white solid (109 mg).

NMR Spectrum: ¹H NMR (400.132 MHz, DMSO-d₆) δ1.07 (3H, m), 1.23 (3H, d), 1.56 (2H, s), 1.67 (2H, s), 3.14 (2H, t), 3.22 (1H, m), 3.30 (3H, s), 3.48 (1H, t), 3.63 (1H, m), 3.76 (1H, d), 3.97 (1H, d), 4.21 (1H, d), 4.57 (1H, s), 6.16 (1H, t), 6.76 (1H, s), 7.50 (2H, d), 8.20 (2H, d), 8.66 (1H, s)

LCMS Spectrum: m/z (ESI+)(M+H)+=460; HPLC tR=1.83 min

mTOR Kinase Assay (Echo): 0.00276 μM

The following compound was made in an analogous fashion from the appropriate boronic ester.

| | | | LCMS | Retention |
|---|---|---|---|---|
| Example | Structure | NAME | MH+ | time (min) |
| 1a | 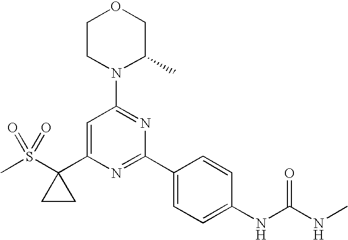 | 3-methyl-1-[4-[4-[(3S)-3-methylmorpholin-4-yl]-6-(1-methylsulfonylcyclopropyl)pyrmidin-2-yl]phenyl]urea | 446 | 1.68 |

### EXAMPLE 1a

¹H NMR (400.132 MHz, DMSO) δ 1.23 (3H, d), 1.31 (3H, s), 1.56 (2H, m), 1.67 (2H, m), 2.66 (3H, d), 3.22 (1H, m), 3.48 (1H, m), 3.63 (1H, m), 3.76 (1H, d), 3.97 (1H, m), 4.21 (1H, d), 4.57 (1H, s), 6.07 (1H, m), 6.76 (1H, s), 7.51 (2H, d), 8.19 (2H, d), 8.75 (1H, s)

mTOR Kinase Assay (Echo): 0.00279 μM

The preparation of 2-chloro-4-[(3S)-3-methylmorpholin-4-yl]-6-(1-methylsulfonylcyclopropyl)pyrimidine is described below.

### 2-Chloro-4-[(3S)-3-methylmorpholin-4-yl]-6-(1-methylsulfonylcyclopropyl)pyrimidine

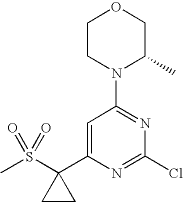

2-Chloro-4-[(3S)-3-methylmorpholin-4-yl]-6-(methylsulfonylmethyl)pyrimidine (1.2 g, 3.9 mmol) was dissolved in DMF (20 mL) and sodium tert-butoxide (755 mg, 7.85 mmol) was added to the reaction, followed by dibromoethane (738 mg, 3.9 mmol). The reaction was stirred at RT for 4 hours then at 60° C. overnight. Further sodium tert-butoxide (378 mg, 3.9 mmol) was added to the reaction, followed by dibromoethane (369 mg, 1.9 mmol) and the reaction stirred at 60° C. a further 24 hours. DCM (20 mL) was added and the reaction washed with 2M aqueous hydrochloric acid (20 mL). The organic phase was dried (MgSO₄), filtered and concentrated in vacuo. The crude solid was chromatographed on silica, eluting with 0-50% ethyl acetate in DCM, to give the desired material (400 mg, 31%).

NMR Spectrum: ¹H NMR (400.132 MHz, DMSO-d₆) δ 1.22 (d, 3H), 1.51 (m, 2H), 1.64 (m, 2H), 3.18 (s, 3H), 3.22 (m, 1H), 3.43 (m, 1H), 3.58 (m, 1H), 3.72 (d, 1H), 3.93 (m, 1H), 4.05 (d, 1H), 4.41 (s, 1H), 6.93 (s, 1H)

LCMS Spectrum: m/z (ESI+)(M+H)+ 332, HPLC tR=1.6 min

### 2-Chloro-4-[(3S)-3-methylmorpholin-4-yl]-6-(methylsulfonylmethyl)pyrimidine

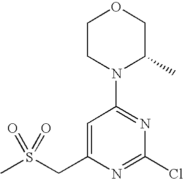

2,4-Dichloro-6-(methylsulfonylmethyl)pyrimidine (30 g, 0.13 mol) was dissolved in dichloromethane and stirred (under nitrogen) at −5° C. Triethylamine (17.4 mL, 0.13 mol) was added to give a clear brown solution. (3S)-3-Methylmorpholine was dissolved in dichloromethane and added dropwise keeping the reaction below −5° C. The cooling bath was then removed and the mixture stirred for 1 hour. The reaction mixture was heated at reflux for 2 hours, then the reaction mixture was washed with water, dried then evaporated. The crude material was purified by preparative HPLC to give the desired material as a solid (19.3 g).

NMR Spectrum: ¹H NMR (400.13 MHz, DMSO-d₆) δ1.21-1.23 (m, 3H), 3.11 (s, 3H), 3.19-3.26 (m, 1H), 3.42-3.49 (m, 1H), 3.58-3.62 (1H, m), 3.73 (d, 1H), 3.92-3.96 (m, 2H), 4.27-4.31 (m, 1H), 4.45 (s, 2H), 6.92 (s, 1H)

LCMS Spectrum: MH+ 306, retention time 1.42 min, Method 5 Min Acid

### 2,4-Dichloro-6-(methylsulfonylmethyl)pyrimidine

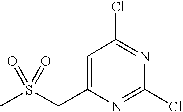

6-(Methylsulfonylmethyl)-1H-pyrimidine-2,4-dione (132 g, 0.65 mol) was added to phosphorus oxychloride (1.2 L) and the mixture heated to reflux for 16 hours, then cooled to room temperature. The excess phosphorus oxychloride was removed in vacuo, the residue azeotroped with toluene (2×500 mL) and dissolved in dichloromethane. This mixture was then poured slowly onto ice (4 L) and stirred for 20 minutes, then extracted with dichloromethane (3×1 L) (the insoluble black material was filtered off and discarded) and ethyl acetate (2×1 L). The extracts were combined, dried, then evaporated to leave the desired material as a dark brown solid (51 g). The material was used without further purification.

NMR Spectrum: ¹H NMR (400.13 MHz, DMSO-d₆) δ3.13 (s, 3H), 4.79 (s, 2H), 7.87 (s, 1H)

LCMS Spectrum: MH+ 239, retention time 1.21 min, Method 5 Min Acid

### 6-(Methylsulfonylmethyl)-1H-pyrimidine-2,4-dione

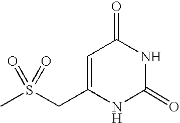

6-(Chloromethyl)-1H-pyrimidine-2,4-dione (175 g, 1.09 mol) was dissolved in DMF (2 L) and methanesulphinic acid sodium salt (133.5 g, 1.31 mol) was added. The reaction was heated to 125° C. for 2 hours then allowed to cool and the suspension filtered and concentrated in vacuo to give a yellow solid. The crude material was washed with water, filtered, then triturated with toluene. The solid was filtered then triturated with isohexane to leave the desired compound as a yellow solid (250 g). The material was used without further purification.

6-(Chloromethyl)-1H-pyrimidine-2,4-dione is a commercially available material.

2-Chloro-4-[(3S)-3-methylmorpholin-4-yl]-6-(1-methylsulfonylcyclopropyl)pyrimidine can also be prepared by the method described below.

### 2-Chloro-4-[(3S)-3-methylmorpholin-4-yl]-6-(1-methylsulfonylcyclopropyl)pyrimidine

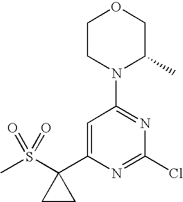

Sodium hydroxide (50% w/w solution) (115 g, 2877.88 mmol) was added to 2-chloro-4-[(3S)-3-methylmorpholin-4-yl]-6-(methylsulfonylmethyl)pyrimidine (16 g, 52.33 mmol), 1,2-dibromoethane (13.53 ml, 156.98 mmol) and tetrabutylammonium bromide (1.687 g, 5.23 mmol) in toluene (128 mL) and the resulting suspension stirred at RT for 4 hours. Water was added and the mixture was extracted twice with toluene. The toluene was dried over MgSO₄, filtered and evaporated. The crude product, 15 g was purified by flash silicachromatography, elution gradient 0 to 20% ethyl acetate in DCM, to give the desired material (13 g) as a white solid which was identical to previous samples.

### 2-Chloro-4-[(3S)-3-methylmorpholin-4-yl]-6-(methylsulfonylmethyl)pyrimidine

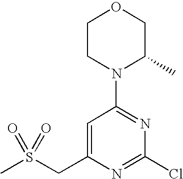

Methanesulfinic acid, sodium salt (11.75 g, 115.11 mmol) was added in one portion to 2-chloro-4-(iodomethyl)-6-[(3S)-3-methylmorpholin-4-yl]pyrimidine (37 g, 104.64 mmol), in acetonitrile (900 mL) and the resulting solution stirred at 85° C. for 24 hours. The organic layers were combined and washed with water (3×100 mL), dried over MgSO₄, filtered, and the solvent was removed by evaporation to give the crude product as a dark brown oil, which solidifed (36 g). The crude solid was purified by flash silicachromatography, elution gradient 0 to 30% ethyl acetate in DCM, to give the desired material (22 g) as a cream solid which was identical to previous samples.

### 2-Chloro-4-(iodomethyl)-6-[(3S)-3-methylmorpholin-4-yl]pyrimidine

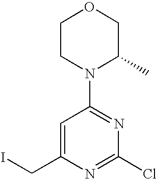

Methanesulfonyl chloride (0.245 mL, 3.14 mmol) was added dropwise over a period of 5 minutes to a solution of triethylamine (0.875 mL, 6.28 mmol) and [2-chloro-6-[(3S)-3-methylmorpholin-4-yl]pyrimidin-4-yl]methanol (510 mg, 2.09 mmol) in DCM (30 mL) at 0° C. under nitrogen. The resulting solution was stirred at RT for 45 minutes. The reaction mixture was diluted with water (20 mL). The organic layer was dried (MgSO₄) and filtered. Sodium Iodide (1569 mg, 10.46 mmol) was added and the reaction was heated to 50° C. for 20 hours. The reaction mixture was filtered and evaporated to afford the desired material (761 mg).

NMR Spectrum: ¹H NMR (400.132 MHz, DMSO) δ 1.19-1.25 (3H, m), 3.18-3.22 (1H, m), 3.40-3.47 (1H, m), 3.57-3.60 (1H, m), 3.71 (1H, d), 3.90-3.94 (1H, m), 3.96-3.98 (1H, m), 4.28-4.32 (3H, m), 6.94 (1H, s).

LCMS Spectrum: m/z (ESI+) (M+H)+=354; HPLC tR=2.10 min.

2-Chloro-4-(iodomethyl)-6-[(3S)-3-methylmorpholin-4-yl]pyrimidine can also be prepared by the dropwise addition of methanesulfonyl chloride (91 mL, 1169.52 mmol) to [2-chloro-6-[(3S)-3-methylmorpholin-4-yl]pyrimidin-4-yl]methanol (190 g, 779.68 mmol) and triethylamine (163 mL, 1169.52 mmol) in DCM (2293 mL) at 0° C. under air. The resulting solution was allowed to warm up slowly to RT over a period of 4 hours. The reaction mixture was quenched with water, extracted with DCM and the organic layer dried over MgSO₄, filtered and evaporated to afford [2-chloro-6-[(3S)-3-methylmorpholin-4-yl]pyrimidin-4-yl]methyl methanesulfonate as a yellow gum (251 g). Sodium iodide (234 g, 1560.07 mmol) was added to this material in acetone (3679 mL) and the resulting suspension stirred at RT for 16 hours. The reaction mixture was evaporated to dryness and redissolved in DCM and washed three times with water then with a saturated aqueous solution of sodium thiosulphate. The organic layer was dried over MgSO₄, filtered and evaporated to afford crude desired product (270 g). This was purified by chromatography to give an off white solid which was further triturated with ether to give the desired material which was identical to previous samples.

### [2-Chloro-6-[(3S)-3-methylmorpholin-4-yl]pyrimidin-4-yl]methanol

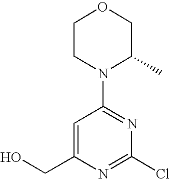

Methyl 2-chloro-6-[(3S)-3-methylmorpholin-4-yl]pyrimidine-4-carboxylate (3.15 g) was dissolved in dry THF (20 mL) and cooled to 0° C. under nitrogen. A solution of lithium borohydride (2.0M in THF, 6.09 mL) was added dropwise and the solution allowed to warm to RT and stirred for 1 hour. The reaction was quenched with water (20 mL) then evaporated to dryness, the residue dissolved in ethyl acetate (150 mL) and washed with water (150 mL) followed by brine (50 mL). The organics were evaporated to dryness to give to the desired material as a white solid (2.44 g).

NMR Spectrum: ¹H NMR (400.132 MHz, DMSO) δ 1.20-1.21 (3H, m), 3.18-3.22 (1H, m), 3.40-3.47 (1H, m), 3.56-3.60 (1H, m), 3.71 (1H, d), 3.91-3.94 (1H, m), 3.98 (1H, d), 4.35 (3H, d), 5.51 (1H, t), 6.74 (1H, s).

Mass Spectrum; M+H⁺ 244.

[2-Chloro-6-[(3S)-3-methylmorpholin-4-yl]pyrimidin-4-yl]methanol can also be prepared by the dropwise addition of lithium borohydride (2M in THF) (454 mL, 908.17 mmol) over a period of 15 minutes to a solution of methyl 2-chloro-6-[(3S)-3-methylmorpholin-4-yl]pyrimidine-4-carboxylate (235 g, 864.92 mmol) in the THF (4701 mL) at 0° C. The mixture was stirred at RT for 2 hours then water (1500 mL) was added slowly. A white solid formed which was decanted off and the THF was removed under vacuum. To the residue was added more water (500 mL), and extracted with ethyl acetate (3×700 mL). The combined organics were washed with brine, dried over MgSO₄, filtered, and concentrated to a white solid which was identical to previous samples.

### Methyl 2-chloro-6-[(3S)-3-methylmorpholin-4-yl]pyrimidine-4-carboxylate

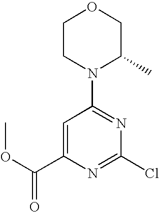

Methyl 2,6-dichloropyrimidine-4-carboxylate (5 g) was dissolved in DCM (120 mL). (3S)-3-Methylmorpholine (2.49 g) dissolved in triethylamine (3.70 mL) and DCM (10 mL) was added dropwise to the solution over 10 minute. The reaction was left to stir at room temperature for 1 hour. The reaction was then evaporated to dryness and dissolved in DCM (300 mL). The organics were washed once with water (150 mL) and dried (MgSO₄), filtered and evaporated. The crude material was chromatographed on silica, eluting with 2.5% methanol in DCM, to give the desired material as a white solid (3.15 g).

NMR Spectrum: ¹H NMR (400.132 MHz, DMSO) δ 1.22-1.24 (3H, m), 3.25 (1H, d), 3.41-3.48 (1H, m), 3.57-3.61 (1H, m), 3.71 (1H, d), 3.87 (3H, s), 3.91-3.95 (1H, m), 4.25 (1H, s), 4.45 (1H, s), 7.29 (1H, s).

Mass Spectrum; M+H⁺ 272.

Methyl 2-chloro-6-[(3S)-3-methylmorpholin-4-yl]pyrimidine-4-carboxylate can also be prepared by the addition of methyl 2,6-dichloropyrimidine-4-carboxylate (250 g, 1207.65 mmol) to the DCM (2500 mL). Triethylamine (185 mL, 1328.41 mmol) was added and the reaction cooled to 0° C. (3S)-3-Methylmorpholine (128 g, 1268.03 mmol) dissolved in DCM (300 mL), was added dropwise over 30 minutes and the mixture stirred at 5° overnight. Water (800 mL) was added, the phases separated and the aquoeus layer extracted with DCM (300 mL). The combined organics were washed with brine (300 mL), dried over MgSO₄, filtered and concentrated to a cream solid. The crude solid was dissolved in hot ethyl acetate (3 volumes) then isohexane (5 volumes) added the mixture left to cool with stirring over the weekend to give the desired material as a solid which was identical to previous samples.

### EXAMPLE 2

### 3-Cyclopropyl-1-[4-[4-[(3S)-3-methylmorpholin-4-yl]-6-(1-methylsulfonylcyclopropyl)pyrimidin-2-yl]phenyl]urea

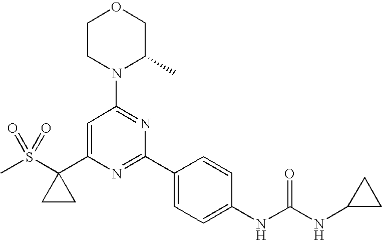

To a solution of phenyl N-[4-[4-[(3S)-3-methylmorpholin-4-yl]-6-(1-methylsulfonylcyclopropyl)pyrimidin-2-yl]phenyl]carbamate (200 mg, 0.39 mmol) in DMF (2 mL) was added triethylamine (0.164 mL, 1.18 mmol) followed by cyclopropylamine (0.136 mL, 1.97 mmol) and the reaction heated at 50° C. for 2 hours.

The solvent was removed under reduced pressure to give the crude product which was purified by flash silica chromatography, elution gradient 0 to 10% (3.5M ammonia in methanol) in DCM, to give the desired product as a white solid (168 mg).

NMR Spectrum: ¹H NMR (400.132 MHz, DMSO-d₆) δ 0.42 (2H, m), 0.65 (2H, m), 1.24 (3H, d), 1.56 (2H, m), 1.67 (2H, m), 2.56 (3H, m), 3.21 (1H, m), 3.48 (1H, m), 3.63 (1H, m), 3.78 (1H, d), 3.97 (1H, m), 4.21 (1H, d), 4.57 (1H, s), 6.43 (1H, d), 6.77 (1H, s), 7.51 (2H, d), 8.20 (2H, d), 8.54 (1H, s)

LCMS Spectrum: m/z (ESI+)(M+H)+=472; HPLC tR=1.93 min.

mTOR Kinase Assay (Echo): 0.0015 μM

The compounds below were prepared in an analogous fashion using the appropriate amine.

| | | | LCMS | Retention |
|---|---|---|---|---|
| Example | Structure | NAME | MH+ | time (min) |
| 2a | 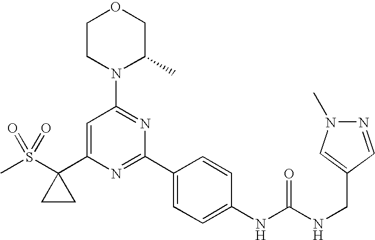 | 1-[4-[4-[(3S)-3-methylmorpholin-4-yl]-6-(1-methylsulfonylcyclopropyl)pyrimidin-2-yl]phenyl]-3-[(1-methylpyrazol-4-yl)methyl]urea | 526 | 1.73 |
| 2b | 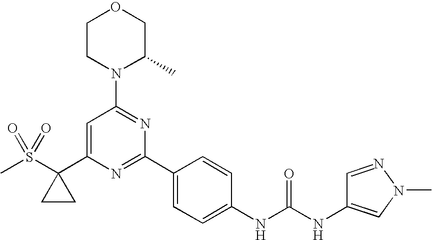 | 1-[4-[4-[(3S)-3-methylmorpholin-4-yl]-6-(1-methylsulfonylcyclopropyl)pyrimidin-2-yl]phenyl]-3-(1-methylpyrazol-4-yl)urea | 512 | 1.75 |
| 2c | 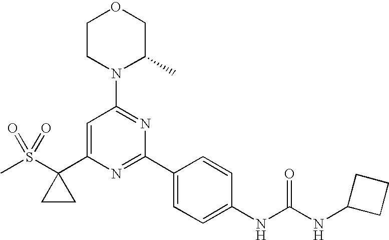 | 3-cyclobutyl-1-[4-[4-[(3S)-3-methylmorpholin-4-yl]-6-(1-methylsulfonylcyclopropyl)pyrimidin-2-yl]phenyl]urea | 486 | 2.21 |
| 2d | 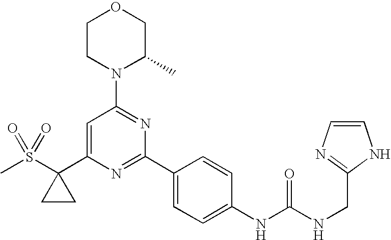 | 3-(1H-imidazol-2-ylmethyl)-1-[4-[4-[(3S)-3-methylmorpholin-4-yl]-6-(1-methylsulfonylcyclopropyl)pyrimidin-2-yl]phenyl]urea | 512 | 1.66 |
| 2e | 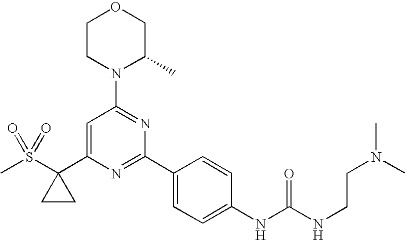 | 3-(2-dimethylaminoethyl)-1-[4-[4-[(3S)-3-methylmorpholin-4-yl]-6-(1-methylsulfonylcyclopropyl)pyrimidin-2-yl]phenyl]urea | 502 | 1.89 |
| 2f | 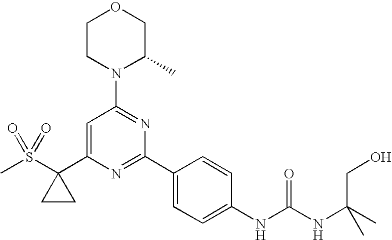 | 3-(1-hydroxy-2-methyl-propan-2-yl)-1-[4-[4-[(3S)-3-methylmorpholin-4-yl]-6-(1-methylsulfonylcyclopropyl)pyrimidin-2-yl]phenyl]urea | 503 | 1.83 |
| 2g | 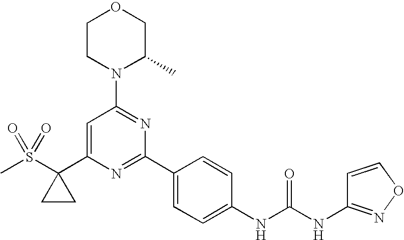 | 1-[4-[4-[(3S)-3-methylmorpholin-4-yl]-6-(1-methylsulfonylcyclopropyl)pyrimidin-2-yl]phenyl]-3-(1,2-oxazol-3-yl)urea | 498 | 2.03 |
| 2h | 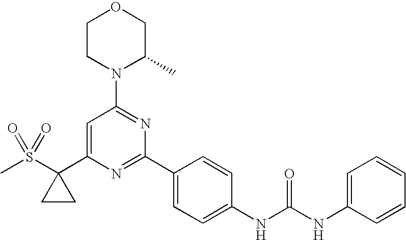 | 1-[4-[4-[(3S)-3-methylmorpholin-4-yl]-6-(1-methylsulfonylcyclopropyl)pyrimidin-2-yl]phenyl]-3-phenyl-urea | 507 | 2.35 |
| 2i | 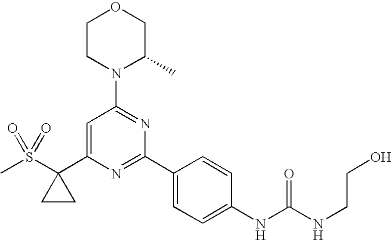 | 3-(2-hydroxyethyl)-1-[4-[4-[(3S)-3-methylmorpholin-4-yl]-6-(1-methylsulfonylcyclopropyl)pyrimidin-2-yl]phenyl]urea | 475 | 1.54 |
| 2j | 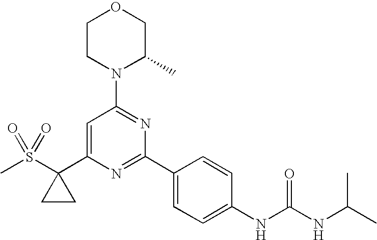 | 1-[4-[4-[(3S)-3-methylmorpholin-4-yl]-6-(1-methylsulfonylcyclopropyl)pyrimidin-2-yl]phenyl]-3-propan-2-yl-urea | 473 | 2.0 |
| 2k | 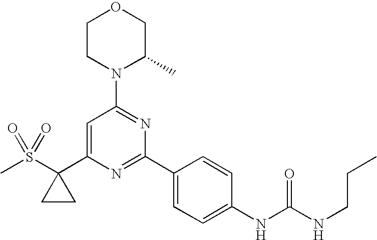 | 1-[4-[4-[(3S)-3-methylmorpholin-4-yl]-6-(1-methylsulfonylcyclopropyl)pyrimidin-2-yl]phenyl]-3-propyl-urea | 473 | 2.02 |
| 2l | 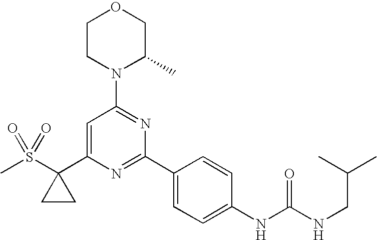 | 1-[4-[4-[(3S)-3-methylmorpholin-4-yl]-6-(1-methylsulfonylcyclopropyl)pyrimidin-2-yl]phenyl]-3-(2-methylpropyl)urea | 487 | 2.18 |
| 2m | 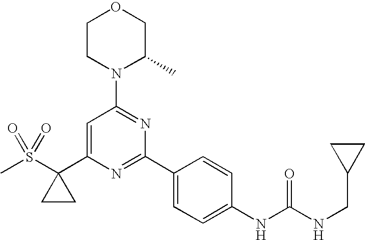 | 3-(cyclopropylmethyl)-1-[4-[4-[(3S)-3-methylmorpholin-4-yl]-6-(1-methylsulfonylcyclopropyl)pyrimidin-2-yl]phenyl]urea | 485 | 2.06 |
| 2n | 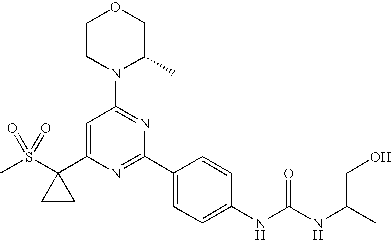 | 3-(1-hydroxypropan-2-yl)-1-[4-[4-[(3S)-3-methylmorpholin-4-yl]-6-(1-methylsulfonylcyclopropyl)pyrimidin-2-yl]phenyl]urea | 489 | 1.63 |
| 2o | 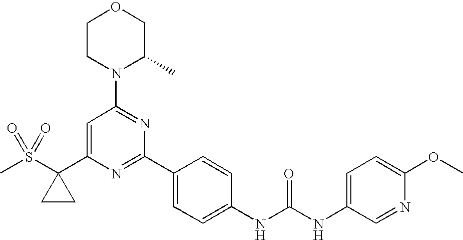 | 3-(6-methoxypyridin-3-yl)-1-[4-[4-[(3S)-3-methylmorpholin-4-yl]-6-(1-methylsulfonylcyclopropyl)pyrimidin-2-yl]phenyl]urea | 538 | 2.11 |
| 2p | 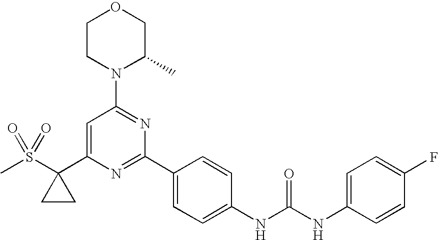 | 3-(4-fluorophenyl)-1-[4-[4-[(3S)-3-methylmorpholin-4-yl]-6-(1-methylsulfonylcyclopropyl)pyrimidin-2-yl]phenyl]urea | 525 | 2.4 |
| 2q | 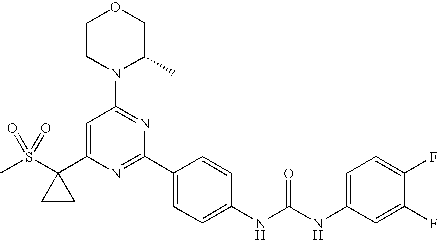 | 3-(3,4-difluorophenyl)-1-[4-[4-[(3S)-3-methylmorpholin-4-yl]-6-(1-methylsulfonylcyclopropyl)pyrimidin-2-yl]phenyl]urea | 543 | 2.52 |
| 2r | 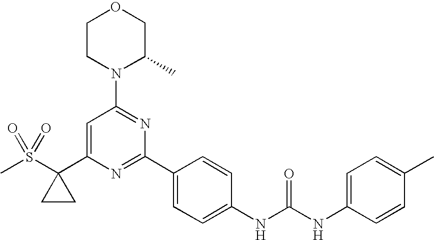 | 1-[4-[4-[(3S)-3-methylmorpholin-4-yl]-6-(1-methylsulfonylcyclopropyl)pyrimidin-2-yl]phenyl]-3-(4-methylphenyl)urea | 521 | 2.49 |
| 2s | 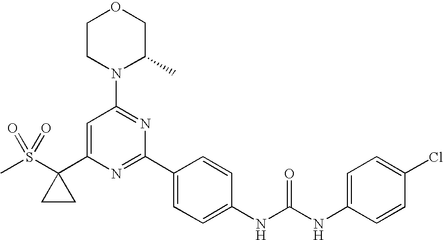 | 3-(4-chlorophenyl)-1-[4-[4-[(3S)-3-methylmorpholin-4-yl]-6-(1-methylsulfonylcyclopropyl)pyrimidin-2-yl]phenyl]urea | 541 | 2.59 |
| 2t | 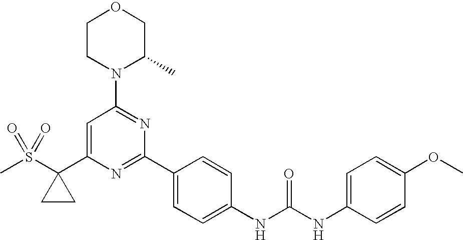 | 3-(4-methoxyphenyl)-1-[4-[4-[(3S)-3-methylmorpholin-4-yl]-6-(1-methylsulfonylcyclopropyl)pyrimidin-2-yl]phenyl]urea | 537 | 2.29 |
| 2u | 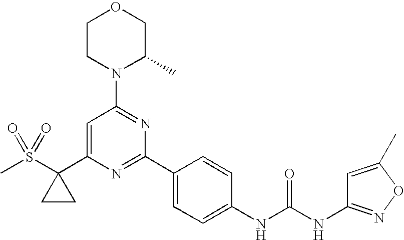 | 1-[4-[4-[(3S)-3-methylmorpholin-4-yl]-6-(1-methylsulfonylcyclopropyl)pyrimidin-2-yl]phenyl]-3-(5-methyl-1,2-oxazol-3-yl)urea | 512 | 2.16 |
| 2v | 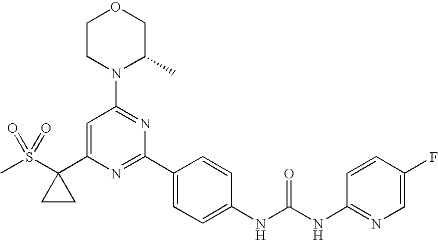 | 3-(5-fluoropyridin-2-yl)-1-[4-[4-[(3S)-3-methylmorpholin-4-yl]-6-(1-methylsulfonylcyclopropyl)pyrimidin-2-yl]phenyl]urea | 527 | 2.35 |
| 2w | 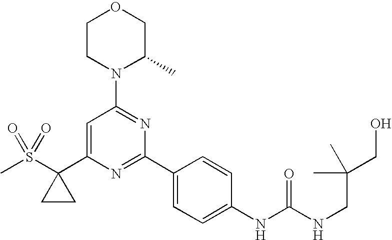 | 3-(3-hydroxy-2,2-dimethylpropyl)-1-[4-[4-[(3S)-3-methylmorpholin-4-yl]-6-(1-methylsulfonylcyclopropyl)pyrimidin-2-yl]phenyl]urea | 518 | 2.05 |
| 2x | 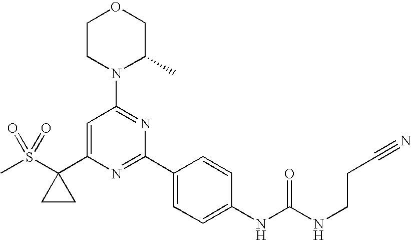 | 3-(2-cyanoethyl)-1-[4-[4-[(3S)-3-methylmorpholin-4-yl]-6-(1-methylsulfonylcyclopropyl)pyrimidin-2-yl]phenyl]urea | 485 | 1.91 |
| 2y | 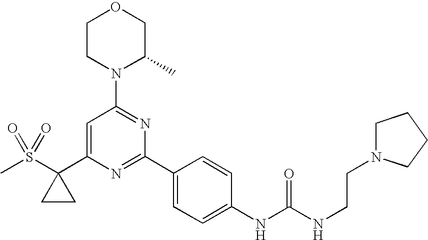 | 1-[4-[4-[(3S)-3-methylmorpholin-4-yl]-6-(1-methylsulfonylcyclopropyl)pyrimidin-2-yl]phenyl]-3-(2-pyrrolidin-1-ylethyl)urea | 529 | 2.21 |
| 2z | 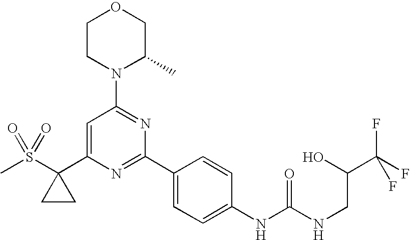 | 1-[4-[4-[(3S)-3-methylmorpholin-4-yl]-6-(1-methylsulfonylcyclopropyl)pyrimidin-2-yl]phenyl]-3-(3,3,3-trifluoro-2-hydroxypropyl)urea | 544 | 2.11 |
| 2aa | 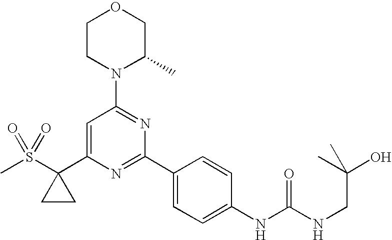 | 3-(2-hydroxy-2-methylpropyl)-1-[4-[4-[(3S)-3-methylmorpholin-4-yl]-6-(1-methylsulfonylcyclopropyl)pyrimidin-2-yl]phenyl]urea | 530 | 1.91 |
| 2ab | 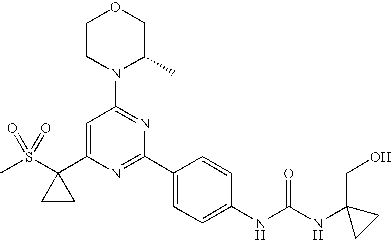 | 3-[1-(hydroxymethyl)cyclopropyl]-1-[4-[4-[(3S)-3-methylmorpholin-4-yl]-6-(1-methylsulfonylcyclopropyl)pyrimidin-2-yl]phenyl]urea | 502 | 1.81 |
| 2ac | 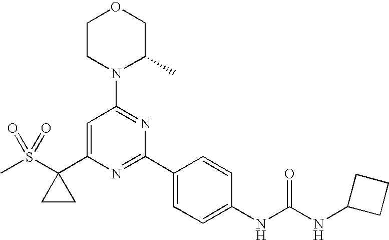 | 1-[4-[4-[(3S)-3-methylmorpholin-4-yl]-6-(1-methylsulfonylcyclopropyl)pyrimidin-2-yl]phenyl]-3-(oxetan-3-yl)urea | 488 | 2.25 |
| 2ad | 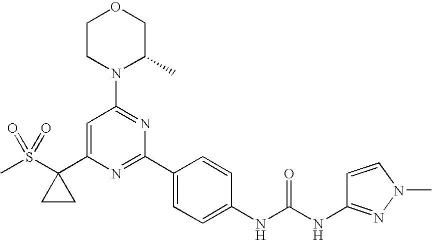 | 1-[4-[4-[(3S)-3-methylmorpholin-4-yl]-6-(1-methylsulfonylcyclopropyl)pyrimidin-2-yl]phenyl]-3-(1-methylpyrazol-3-yl)urea | 512 | 2.11 |
| 2ae | 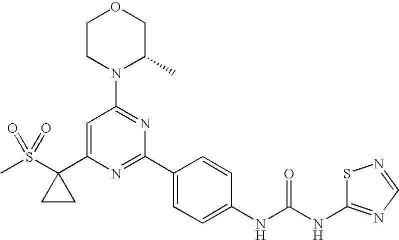 | 1-[4-[4-[(3S)-3-methylmorpholin-4-yl]-6-(1-methylsulfonylcyclopropyl)pyrimidin-2-yl]phenyl]-3-(1,2,4-thiadiazol-5-yl)urea | 515 | 1.43 |
| 2af | 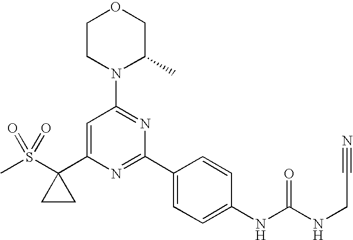 | 3-(cyanomethyl)-1-[4-[4-[(3S)-3-methylmorpholin-4-yl]-6-(1-methylsulfonylcyclopropyl)pyrimidin-2-yl]phenyl]urea | 471 | 1.54 |
| 2ag | 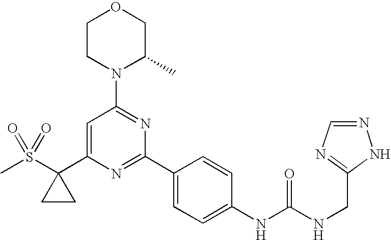 | 1-[4-[4-[(3S)-3-methylmorpholin-4-yl]-6-(1-methylsulfonylcyclopropyl)pyrimidin-2-yl]phenyl]-3-(2H-1,2,4-triazol-3-ylmethyl)urea | 513 | 1.58 |

Both Example 2 and Example 1a can be prepared in an analogous fashion to that described above but using THF as a solvent. Example 1a can also be prepared in an analogous fashion to that described above but using NMP as a solvent.

### EXAMPLE 2a

¹H NMR (400.132 MHz, DMSO-d₆) δ 1.22 (3H, d), 1.56 (2H, s), 1.67 (2H, s), 3.21 (1H, m), 3.48 (1H, t), 3.63 (1H, d), 3.76 (4H, m), 3.80 (3H, s), 3.97 (1H, d), 4.13 (2H, d), 4.20 (1H, s), 4.57 (1H, s), 6.42 (1H, t), 6.77 (1H, s), 7.35 (1H, s), 7.51 (2H, d), 7.59 (1H, s), 8.20 (2H, d), 8.70 (1H, s)

mTOR Kinase Assay (Echo): 0.0932 μM

### EXAMPLE 2b

¹H NMR (400.132 MHz, DMSO-d₆) δ 1.24 (3H, d), 1.55-1.58 (2H, m), 1.66-1.69 (2H, m), 3.21 (1H, dt), 3.27-3.29 (1H, m), 3.29 (3H, s), 3.49 (1H, dt), 3.64 (1H, dd), 3.79 (3H, s), 3.98 (1H, dd), 4.22 (1H, d), 4.58 (1H, s), 6.78 (1H, s), 7.38 (1H, s), 7.55 (2H, d), 7.76 (1H, s), 8.23 (2H, d), 8.38 (1H, s), 8.84 (1H, s)

mTOR Kinase Assay (Echo): 0.000169 μM

### EXAMPLE 2c

¹H NMR (400.132 MHz, DMSO-d₆) δ 1.23-1.24 (3H, d), 1.54-1.69 (6H, m), 1.81-1.91 (2H, m), 2.18-2.25 (2H, m), 3.17-3.24 (1H, td), 3.29 (3H, s), 3.45-3.52 (1H, td), 3.62-3.65 (1H, dd), 3.75-3.78 (1H, d), 3.96-3.99 (1H, dd), 4.10-4.23 (2H, m), 4.57 (1H, bs), 6.45-6.47 (1H, d), 6.77 (1H, s), 7.47-7.50 (2H, d), 8.18-8.21 (2H, d), 8.57 (1H, s).

mTOR Kinase Assay (Echo): 0.00121 μM

### EXAMPLE 2d

¹H NMR (400.132 MHz, DMSO-d₆) δ 1.23-1.25 (3H, d), 1.55-1.66 (2H, q), 1.67-1.69 (2H, q), 3.17-3.25 (1H, td), 3.30 (3H, s), 3.45-3.52 (1H, td), 3.62-3.65 (1H, dd), 3.75-3.78 (1H, d), 3.96-3.99 (1H, dd), 4.20-4.23 (1H, d), 4.32-4.34 (2H, d), 4.57 (1H, bs), 6.61-6.64 (1H, t), 6.77 (1H, s), 6.77 (2H, bs), 7.51-7.54 (2H, d), 8.21-8.22 (2H, d), 8.94 (1H, s), 11.84 (1H, bs).

mTOR Kinase Assay (Echo): 0.0239 μM

### EXAMPLE 2e

¹H NMR (400.132 MHz, DMSO-d₆) δ 1.23-1.24 (3H, d), 1.54-1.58 (2H, q), 1.66-1.69 (2H, q), 2.19 (6H, s), 2.33-2.36 (2H, t), 3.18-3.22 (2H, t), 3.20-3.25 (1H, td), 3.45-3.52 (1H, td), 3.62-3.65 (1H, dd), 3.75-3.78 (1H, d), 3.96-3.99 (1H, dd), 4.20-4.23 (1H, d), 4.57 (1H, bs), 6.15-6.18 (1H, t), 6.77 (1H, s), 7.49-7.51 (2H, d), 8.19-8.21 (2H, d), 8.90 (1H, s).

Note: methyl signal obscured by water peak at 3.29.

mTOR Kinase Assay (Echo): 0.279 μM

### EXAMPLE 2f

¹H NMR (400.132 MHz, DMSO-d₆) δ 1.23-1.25(9H, d), 1.54-1.58 (2H, q), 1.66-1.69 (2H, q), 3.17-3.25 (1H, td), 3.39-3.40 (2H, d), 3.45-3.52 (1H, td), 3.62-3.65 (1H, dd), 3.75-3.78 (1H, d), 3.96-4.00 (1H, dd), 4.20-4.23 (1H, d), 4.56 (1H, bs), 4.94-4.97 (1H, t), 6.01 (1H, s), 6.77 (1H, s), 7.45-7.47 (2H, d), 8.18-8.20 (2H, d), 8.74 (1H, s). Note: methyl signal obscured by water peak at 3.29.

mTOR Kinase Assay (Echo): 0.00433 μM

### EXAMPLE 2g

¹H NMR (400.132 MHz, DMSO-d₆) δ 1.24-1.25 (3H, d), 1.56-1.59 (2H, q), 1.67-1.70 (2H, q), 3.19-3.26 (1H, td), 3.46-3.53 (1H, td), 3.62-3.66 (1H, dd), 3.76-3.79 (1H, d), 3.97-4.00 (1H, dd), 4.21-4.24 (1H, d), 4.58 (1H, bs), 6.81 (1H, s), 6.87-6.88 (1H, d), 7.57-7.59 (2H, d), 8.27-8.29 (2H, d), 8.75-8.76 (1H, d), 9.08 (1H,s), 9.62 (1H, s). Note: methyl signal obscured by water peak at 3.29.

mTOR Kinase Assay (Echo): 0.000137 μM

### EXAMPLE 2h

¹H NMR (400.132 MHz, DMSO-d₆) δ 1.24-1.26 (3H, d), 1.56-1.59 (2H, q), 1.67-1.70 (2H, q), 3.18-3.26 (1H, td), 3.31 (3H, s), 3.46-3.53 (1H, td), 3.63-3.66 (1H, dd), 3.76-3.79 (1H, d), 3.96-4.00 (1H, dd), 4.21-4.24 (1H, d), 4.59 (1H, bs), 6.79 (1H, s), 6.98-7.01 (1H, t), 7.28-7.32 (2H, t), 7.46-7.48 (2H, d), 7.57-7.59 (2H, d), 8.25-8.27 (2H, d), 8.71 (1H, s), 8.92 (1H, s).

mTOR Kinase Assay (Echo): 0.000272 μM

### EXAMPLE 2i

¹H NMR (400.132 MHz, DMSO-d₆) δ 1.23-1.24 (3H, d), 1.54-1.58 (2H, q), 1.65-1.70 (2H, q), 3.16-3.25 (1H, td), 3.16-3.21 (2H, q), 3.45-3.52 (1H, td), 3.45-3.49 (2H, q), 3.62-3.65 (1H, dd), 3.75-3.78 (1H, d), 3.96-3.99 (1H, dd), 4.20-4.23 (1H, d), 4.57 (1H, bs), 4.72-4.74 (1H, t), 6.25-6.27 (1H, t), 6.77 (1H, s), 7.49-7.51 (2H, d), 8.19-8.21 (2H, d), 8.82 (1H, s). Note: methyl signal obscured by water peak at 3.29.

mTOR Kinase Assay (Echo): 0.00207 μM

### EXAMPLE 2j

¹H NMR (400.132 MHz, DMSO-d₆) δ 1.11-1.12 (6H, d), 1.23-1.24 (3H, d), 1.54-1.58 (2H, q), 1.66-1.69 (2H, q), 3.17-3.25 (1H, td), 3.45-3.52 (1H, td), 3.62-3.65 (1H, dd), 3.74-3.82 (2H, m), 3.96-3.99 (1H, dd), 4.20-4.23 (1H, d), 4.57 (1H, bs), 6.06-6.07 (1H, d), 6.77 (1H, s), 7.48-7.50 (2H, d), 8.19-8.21 (2H, d), 8.54 (1H, s). Note: methyl signal obscured by water peak at 3.29.

mTOR Kinase Assay (Echo): 0.012 μM

### EXAMPLE 2k

¹H NMR (400.132 MHz, DMSO-d₆) δ 0.87-0.91 (3H, t), 1.23-1.25 (3H, d), 1.42-1.51 (2H, m), 1.54-1.58 (2H, q), 1.66-1.69 (2H, q), 3.05-3.09 (2H, q), 3.17-3.25 (1H, td), 3.45-3.52 (1H, td), 3.61-3.65 (1H, dd), 3.75-3.78 (1H, d), 3.96-3.99 (1H, dd), 4.20-4.23 (1H, d), 4.57 (1H, bs), 6.19-6.22 (1H, t), 6.77 (1H, s), 7.49-7.51 (2H, d), 8.19-8.21 (2H, d), 8.66 (1H, s). Note: methyl signal obscured by water peak at 3.29.

mTOR Kinase Assay (Echo): 0.00406 μM

### EXAMPLE 2l

¹H NMR (400.132 MHz, DMSO-d₆) δ 0.88-0.90 (6H, d), 1.23-1.25 (3H, d), 1.54-1.58 (2H, q), 1.66-1.68 (2H, q), 1.67-1.76 (1H, m), 2.93-2.96 (2H, t), 3.17-3.25 (1H, td), 3.45-3.52 (1H, td), 3.62-3.65 (1H, dd), 3.75-3.78 (1H, d), 3.96-3.99 (1H, dd), 4.20-4.23 (1H, d), 4.57 (1H, bs), 6.24-6.27 (1H, t), 6.77 (1H, s), 7.49-7.51 (2H, d), 8.19-8.21 (2H, d), 8.66 (1H, s). Note: methyl signal obscured by water peak at 3.29.

mTOR Kinase Assay (Echo): 0.0116 μM

### EXAMPLE 2m

¹H NMR (400.132 MHz, DMSO-d₆) δ 0.18-0.22 (2H, m), 0.42-0.46 (2H, m), 0.93-1.00 (1H, m), 1.23-1.25 (3H, d), 1.54-1.58 (2H, q), 1.66-1.69 (2H, q), 2.98-3.01 (2H, t), 3.17-3.25 (1H, td), 3.45-3.52 (1H, td), 3.62-3.65 (1H, dd), 3.75-3.78 (1H, d), 3.96-3.99 (1H, dd), 4.20-4.23 (1H, d), 4.57 (1H, bs), 6.25-6.28 (1H, t), 6.77 (1H, s), 7.49-7.51 (2H, d), 8.19-8.21 (2H, d), 8.70 (1H, s). Note: methyl signal obscured by water peak at 3.29.

mTOR Kinase Assay (Echo): 0.00589 μM

### EXAMPLE 2n

¹H NMR (400.132 MHz, DMSO-d₆) δ 1.08-1.10 (3H, d), 1.23-1.25 (3H, d), 1.54-1.57 (2H, q), 1.66-1.69 (2H, q), 2.98-3.01 (2H, t), 3.18-3.25 (1H, td), 3.30 (3H, s), 3.34-3.43 (2H, m), 3.45-3.52 (1H, td), 3.62-3.65 (1H, dd), 3.69-3.75 (1H, m), 3.75-3.78 (1H, d), 3.96-3.99 (1H, dd), 4.20-4.23 (1H, d), 4.57 (1H, bs), 4.77-4.80 (1H, t), 6.09-6.11 (1H, d), 6.77 (1H, s), 7.48-7.50 (2H, d), 8.19-8.21 (2H, d), 8.72 (1H, s).

mTOR Kinase Assay (Echo): 0.00844 μM

### EXAMPLE 2o

¹H NMR (400.132 MHz, DMSO-d₆) δ 1.23-1.25 (3H, d), 1.56-1.59 (2H, q), 1.67-1.70 (2H, q), 3.18-3.26 (1H, td), 3.30 (3H, s), 3.46-3.53 (1H, td), 3.62-3.66 (1H, dd), 3.76-3.79 (1H, d), 3.83 (3H, s), 3.96-4.00 (1H, dd), 4.21-4.24 (1H, d), 4.58 (1H, bs), 6.79(s, 1H), 6.79-6.81 (1H, d), 7.56-7.58 (2H, d), 7.83-7.86 (1H, dd), 8.21-8.22 (1H, d), 8.25-8.27 (2H, d), 8.62 (1H, s), 8.98 (1H, s).

mTOR Kinase Assay (Echo): 0.000851 μM

### EXAMPLE 2p

¹H NMR (400.132 MHz, DMSO-d₆) δ 1.23-1.25 (3H, d), 1.55-1.58 (2H, q), 1.66-1.69 (2H, q), 3.18-3.25 (1H, td), 3.30 (3H, s), 3.46-3.52 (1H, td), 3.62-3.66 (1H, dd), 3.76-3.78 (1H, d), 3.96-4.00 (1H, dd), 4.21-4.24 (1H, d), 4.58 (1H, bs), 6.79(s, 1H), 7.11-7.16 (2H, t), 7.46-7.50 (2H, m), 7.56-7.58 (2H, d), 8.24-8.27 (2H, d), 8.74 (1H, s), 8.92 (1H, s).

mTOR Kinase Assay (Echo): 0.0027 μM

### EXAMPLE 2q

¹H NMR (400.132 MHz, DMSO-d₆) δ 1.24-1.25 (3H, d), 1.56-1.59 (2H, q), 1.67-1.70 (2H, q), 3.18-3.26 (1H, td), 3.30 (3H, s), 3.46-3.53 (1H, td), 3.62-3.66 (1H, dd), 3.76-3.79 (1H, d), 3.96-4.00 (1H, dd), 4.21-4.24 (1H, d), 4.58 (1H, bs), 6.80(s, 1H), 7.14-7.17 (1H, m), 7.32-7.39 (1H, q), 7.56-7.59 (2H, d), 7.65-7.71 (1H, qd), 8.26-8.28 (2H, d), 8.93 (1H, s), 9.00 (1H, s).

mTOR Kinase Assay (Echo): 0.001 μM

### EXAMPLE 2r

¹H NMR (400.132 MHz, DMSO-d₆) δ 1.24-1.25 (3H, d), 1.56-1.59 (2H, q), 1.67-1.70 (2H, q), 2.26 (3H, s), 3.18-3.26 (1H, td), 3.30 (3H, s), 3.46-3.53 (1H, td), 3.62-3.66 (1H, dd), 3.76-3.79 (1H, d), 3.96-4.00 (1H, dd), 4.21-4.24 (1H, d), 4.58 (1H, bs), 6.79(s, 1H), 7.09-7.12 (2H, d), 7.34-7.36 (2H, d), 7.56-7.58 (2H, d), 8.24-8.27 (2H, d), 8.59 (1H, s), 8.87 (1H, s).

mTOR Kinase Assay (Echo): 0.00066 μM

### EXAMPLE 2s

¹H NMR (400.132 MHz, DMSO-d₆) δ 1.24-1.25 (3H, d), 1.56-1.59 (2H, q), 1.67-1.70 (2H, q), 3.18-3.26 (1H, td), 3.46-3.53 (1H, td), 3.62-3.66 (1H, dd), 3.76-3.79 (1H, d), 3.96-4.00 (1H, dd), 4.21-4.24 (1H, d), 4.58 (1H, bs), 6.79(s, 1H), 7.33-7.36 (2H, d), 7.50-7.52 (2H, d), 7.56-7.59 (2H, d), 8.25-8.27 (2H, d), 8.87 (1H, s), 8.97 (1H, s). Note: methyl signal obscured by water peak at 3.29

mTOR Kinase Assay (Echo): 0.00138 μM

### EXAMPLE 2t

¹H NMR (400.132 MHz, DMSO-d₆) δ 1.24-1.25 (3H, d), 1.56-1.58 (2H, q), 1.67-1.69 (2H, q), 3.18-3.25 (1H, td), 3.46-3.53 (1H, td), 3.62-3.66 (1H, dd), 3.73 (3H, s), 3.76-3.79 (1H, d), 3.96-4.00 (1H, dd), 4.21-4.24 (1H, d), 4.58 (1H, bs), 6.79(s, 1H), 6.88-6.90 (2H, d), 7.36-7.38 (2H, d), 7.55-7.57 (2H, d), 8.24-8.26 (2H, d), 8.51 (1H, s), 8.84 (1H, s). Note: methyl signal obscured by water peak at 3.29

mTOR Kinase Assay (Echo): 0.00145 μM

### EXAMPLE 2u

¹H NMR (400.132 MHz, DMSO-d₆) δ 1.24-1.25 (3H, d), 1.56-1.59 (2H, q), 1.67-1.70 (2H, q), 2.38 (3H, s), 3.18-3.26 (1H, td), 3.46-3.53 (1H, td), 3.62-3.66 (1H, dd), 3.76-3.79 (1H, d), 3.96-4.00 (1H, dd), 4.21-4.24 (1H, d), 4.58 (1H, bs), 6.57 (1H, s), 6.80 (s, 1H), 7.56-7.58 (2H, d), 8.27-8.29 (2H, d), 9.06 (1H, s), 9.47 (1H, s). Note: methyl signal obscured by water peak at 3.29

mTOR Kinase Assay (Echo): 0.00118 μM

### EXAMPLE 2v

¹H NMR (400.132 MHz, DMSO-d₆) δ 1.24-1.26 (3H, d), 1.56-1.59 (2H, q), 1.67-1.70 (2H, q), 3.19-3.26 (1H, td), 3.31 (3H, s), 3.46-3.53 (1H, td), 3.63-3.66 (1H, dd), 3.76-3.79 (1H, d), 3.97-4.00 (1H, dd), 4.22-4.25 (1H, d), 4.58 (1H, bs), 6.80 (1H, s), 7.60-7.63 (2H, d), 7.72-7.77 (1H, td), 7.79-7.82 (1H, dd), 8.28-8.30 (3H, m), 9.40 (1H, s), 9.89 (1H, s).

mTOR Kinase Assay (Echo): 0.00866 μM

### EXAMPLE 2w

¹H NMR (400.132 MHz, DMSO-d₆) δ 0.82 (6H, s), 1.23 (3H, d), 1.54-1.57 (2H, m), 3.00 (2H, d), 3.15 (2H, d), 3.18-3.24 (1H, m), 3.29 (3H, s), 3.48 (1H, dt), 3.63 (1H, dd), 3.76 (1H, d), 3.97 (1H, dd), 4.17 (1H, d), 4.21 (1H, d), 4.55-4.62 (2H, m), 6.24 (1H, t), 6.76 (1H, s), 7.49 (2H, d), 8.20 (2H, d), 8.77 (1H, s).

mTOR Kinase Assay (Echo): 0.0685 μM

### EXAMPLE 2x

¹H NMR (400.132 MHz, DMSO-d₆) δ 1.23 (3H, d), 1.54-1.57 (2H, m), 1.66-1.68 (2H, m), 2.70 (2H, t), 3.21 (1H, dt), 3.27 (3H, s), 3.35-3.40 (2H, m), 3.48 (1H, dt), 3.64 (1H, dd), 3.76 (1H, d), 3.97 (1H, dd), 4.22 (1H, d), 4.57 (1H, s), 6.53 (1H, t), 6.77 (1H, s), 7.52 (2H, d), 8.21 (2H, d), 8.93 (1H, s).

mTOR Kinase Assay (Echo): 0.00164 μM

### EXAMPLE 2y

¹H NMR (400.132 MHz, DMSO-d₆) δ 1.23 (3H, d), 1.54-1.57 (2H, m), 1.65-1.67 (2H, m), 1.70-1.73 (4H, m), 2.46-2.50 (4H, m), 3.20-3.24 (4H, m), 3.27 (3H, s), 3.48 (1H, dt), 3.63 (1H, dd), 3.76 (1H, d), 3.95-3.99 (1H, m), 3.97 (1H, dd), 4.21 (1H, d), 4.56 (1H, s), 6.19 (1H, t), 6.76 (1H, s), 7.50 (2H, d), 8.19 (2H, d), 8.88 (1H, s).

mTOR Kinase Assay (Echo): 0.23 μM

### EXAMPLE 2z

¹H NMR (400.132 MHz, DMSO-d₆) δ 1.23 (3H, d), 1.54-1.57 (2H, m), 1.66-1.68 (2H, m), 3.14-3.27 (1H, m), 3.29 (3H, s), 3.45-3.56 (2H, m), 3.63 (1H, dd), 3.76 (1H, d), 3.97 (1H, dd), 4.05-4.11 (1H, m), 4.21 (1H, d), 4.57 (1H, s), 6.39 (1H, t), 6.49 (1H, d), 6.77 (1H, s), 7.51 (2H, d), 8.21 (2H, d), 8.94 (1H, s).

mTOR Kinase Assay (Echo): 0.0181 μM

### EXAMPLE 2aa

¹H NMR (400.132 MHz, DMSO-d₆) δ 1.11 (6H, s), 1.23 (3H, d), 1.54-1.57 (2H, m), 1.66-1.68 (2H, m), 3.06 (2H, d), 3.18 (1H, d), 3.22 (′H, dd), 3.29 (3H, s), 3.48 (1H, dt), 3.63 (1H, dd), 3.76 (1H, d), 3.97 (1H, dd), 4.21 (1H, d), 4.52 (1H, s), 4.57 (1H, s), 6.25 (1H, t), 6.76 (1H, s), 7.49 (2H, d), 8.20 (2H, d), 8.90 (1H, s).

mTOR Kinase Assay (Echo): 0.0274 μM

### EXAMPLE 2ab

¹H NMR (400.132 MHz, DMSO-d₆) δ 0.63-0.67 (2H, m), 0.69-0.73 (2H, m), 1.23 (3H, d), 1.54-1.57 (2H, m), 1.66-1.68 (2H, m), 3.18-3.24 (1H, m), 3.29 (3H, s), 3.43-3.52 (3H, m), 3.63 (1H, dd), 3.76 (1H, d), 3.97 (1H, dd), 4.21 (1H, d), 4.56 (1H, s), 4.83 (1H, s), 6.57 (1H, s), 6.77 (1H, s), 7.48 (2H, d), 8.20 (2H, d), 8.69 (1H, s).

mTOR Kinase Assay (Echo): 0.0553 μM

### EXAMPLE 2ac

¹H NMR (400.132 MHz, DMSO-d₆) δ 1.23 (3H, d), 1.54-1.57 (2H, m), 1.66-1.68 (2H, m), 3.17-3.24 (1H, m), 3.29 (3H, s), 3.48 (1H, dt), 3.63 (1H, dd), 3.76 (1H, d), 3.97 (1H, dd), 4.21 (1H, d), 4.44 (2H, t), 4.56 (1H, s), 4.72-4.82 (3H, m), 6.77 (1H, s), 6.95 (1H, d), 7.50 (2H, d), 8.20 (2H, d), 8.78 (1H, s).

mTOR Kinase Assay (Echo): 0.00641 μM

### EXAMPLE 2ad

¹H NMR (400.132 MHz, DMSO-d₆) δ 1.24 (3H, d), 1.55-1.58 (2H, m), 1.66-1.69 (2H, m), 3.17-3.25 (1H, m), 3.27 (3H, s), 3.49 (1H, dt), 3.64 (1H, dd), 3.74 (3H, s), 3.77 (1H, d), 3.98 (1H, dd), 4.22 (1H, d), 4.57 (1H, s), 6.25 (1H, d), 6.79 (1H, s), 7.54 (1H, t), 7.57 (2H, d), 8.25 (2H, d), 8.92 (1H, s), 9.18 (1H, s).

mTOR Kinase Assay (Echo): 0.000705 μM

### EXAMPLE 2ae

¹H NMR (400.132 MHz, DMSO-d₆) δ 1.25 (3H, d), 1.56-1.59 (2H, m), 1.67-1.70 (2H, m), 3.17-3.23 (1H, m), 3.27 (3H, s), 3.49 (1H, dt), 3.64 (1H, dd), 3.77 (1H, d), 3.98 (1H, dd), 4.24 (1H, d), 4.58 (1H, s), 6.81 (1H, s), 7.64 (2H, d), 8.30 (2H, d), 8.35 (1H, s), 9.46 (1H, s).

mTOR Kinase Assay (Echo): 0.00072 μM

### EXAMPLE 2af

¹H NMR (400.132 MHz, DMSO-d₆) δ 1.25 (3H, d), 1.57-1.60 (2H, m), 1.68-1.71 (2H, m), 3.18-3.24 (1H, m), 3.27 (3H, s), 3.50 (1H, d), 3.65 (1H, d), 3.77 (1H, d), 3.98 (1H, d), 4.08 (2H, s), 4.25 (1H, d), 4.61 (1H, s), 6.86 (1H, s), 7.55 (2H, d), 8.14 (1H, s), 8.35 (2H, d), 8.44 (1H, d).

mTOR Kinase Assay (Echo): 0.0462 μM

### EXAMPLE 2ag

¹H NMR (400.132 MHz, DMSO-d₆) δ 1.23 (3H, d), 1.54-1.57 (2H, m), 1.66-1.68 (2H, m), 3.18 (1H, d), 3.22 (1H, dd), 3.49 (1H, dt), 3.63 (1H, dd), 3.76 (1H, d), 3.97 (1H, dd), 4.21 (1H, d), 4.38 (2H, s), 4.44 (1H, s), 4.57 (1H, s), 6.66 (1H, d), 6.77 (1H, s), 7.52 (2H, d), 8.19 (1H, d), 8.21 (2H, d), 8.95 (1H, s), 13.83 (1H, s), 13.83 (1H, s).

mTOR Kinase Assay (Echo): 0.0149 μM

The preparation of phenyl N-[4-[4-[(3S)-3-methylmorpholin-4-yl]-6-(1-methylsulfonylcyclopropyl)pyrimidin-2-yl]phenyl]carbamate is described below:

### Phenyl N-[4-[4-[(3S)-3-methylmorpholin-4-yl]-6-(1-methylsulfonylcyclopropyl)pyrimidin-2-yl]phenyl]carbamate

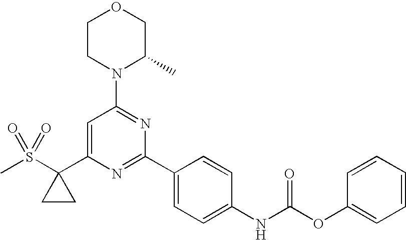

To a solution of 4-[4-[(3S)-3-methylmorpholin-4-yl]-6-(1-methylsulfonylcyclopropyl)pyrimidin-2-yl]aniline (1.35 g, 3.48 mmol) in 1,4-Dioxane (17.4 mL) was added sodium bicarbonate (0.438 g, 5.21 mmol) and phenyl chloroformate (0.437 mL, 3.48 mmol) and the reaction stirred at RT for 2 hours. The reaction mixture was diluted with DCM (20 mL), and washed with water (20 mL), the organic layer dried (MgSO4), filtered and evaporated. The crude product was purified by flash silica chromatography, elution gradient 0 to 40% ethyl acetate in DCM, to give the desired material as a white solid (1.058 g).

NMR Spectrum: ¹H NMR (400.132 MHz, DMSO) δ 1.24 (3H, d), 1.57 (2H, m), 1.68 (2H, m), 3.23 (1H, m), 3.49 (1H, m), 3.58 (3H, s), 3.64 (1H, m), 3.77 (1H, d), 3.97 (1H, m), 4.23 (1H, d), 4.58 (1H, s), 6.81 (1H, s), 7.25 (2H, d), 7.30 (1H, d), 7.45 (2H, m), 7.64 (2H, d), 8.30 (2H, d), 10.44 (1H, s)

LCMS Spectrum: m/z (ESI+) (M+H)+=509; HPLC tR=2.48 min.

### 4-[4-[(3S)-3-Methylmorpholin-4-yl]-6-(1-methylsulfonylcyclopropyl)pyrimidin-2-yl]aniline

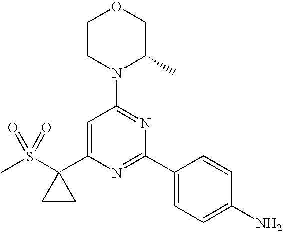

To a solution of 2-chloro-4-[(3S)-3-methylmorpholin-4-yl]-6-(1-methylsulfonylcyclopropyl)pyrimidine (1.52 g, 4.58 mmol) in DMF (0.24 mL), DME (9.33 mL), water (4.0 mL) and ethanol (2.67 mL) was added 4-(tert-butoxycarbonylamino)phenylboronic acid (1.629 g, 6.87 mmol), sodium carbonate (5.73 mL, 11.45 mmol), and dichlorobis(triphenylphosphine)palladium(II) (0.161 g, 0.23 mmol) and the suspension heated at 80° C. for 2 hours. The reaction mixture was cooled to RT, diluted with ethyl acetate (10 mL) and washed with water (10 mL). The organic layer was dried (MgSO₄), filtered and evaporated. The crude product was dissolved in DCM (6.67 mL) and trifluoroacetic acid (0.353 mL, 4.58 mmol) added and the reaction was stirred at RT for 16 hours. The crude product was purified by flash silica chromatography, elution gradient 0 to 10% (7.5N ammonia in methanol) in DCM, to give the desired material as a beige solid (1.283 g).

NMR Spectrum: ¹H NMR (400.132 MHz, DMSO) δ 1.24 (3H, d), 1.55 (2H, m), 1.67 (2H, m), 3.23 (1H, m), 3.27 (3H, s), 3.47 (1H, m), 3.63 (1H, m), 3.77 (1H, d), 3.97 (1H, m), 4.24 (1H, s), 4.58 (1H, s), 5.75 (1H, s), 6.68 (2H, d), 8.04 (2H, d)

LCMS Spectrum: m/z (ESI+) (M+H)+=389; HPLC tR=1.82 min.

### EXAMPLE 3

### 3-Cyclopropyl-1-[4-[4-(1-cyclopropylsulfonylcyclopropyl)-6-[(3S)-3-methylmorpholin-4-yl]pyrimidin-2-yl]phenyl]urea

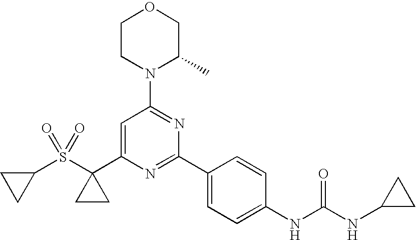

[4-(3-Cyclopropylureido)phenyl]boronic acid, pinacol ester (199 mg, 0.66 mmol), 2-chloro-4-(1-cyclopropylsulfonylcyclopropyl)-6-[(3S)-3-methylmorpholin-4-yl]pyrimidine (189 mg, 0.53 mmol), dichlorobis(triphenylphosphine)palladium(II) (37.1 mg, 0.05 mmol) and sodium carbonate (1.32 mL, 2.64 mmol) were dissolved in a solution of 18% DMF in DME:Water:Ethanol 7:3:2 (4 mL) and sealed into a microwave tube. The reaction was heated to 100° C. for 20 minutes in the microwave reactor and cooled to RT. The crude product was purified by ion exchange chromatography, using an SCX column. The desired product was eluted from the column using 7M ammonia in methanol and pure fractions were evaporated to dryness to afford a crude product. The crude product was purified by preparative HPLC using decreasingly polar mixtures of water (containing 1% NH3) and MeCN as eluents. Fractions containing the desired compound were evaporated to dryness to afford the desired material as a colourless gum (69.0 mg).

NMR Spectrum: ¹H NMR (400.132 MHz, DMSO) δ 0.40-0.44 (2H, m), 0.62-0.67 (2H, m), 0.91-0.97 (1H, m), 0.94 (1H, s), 1.02-1.04 (2H, m), 1.23 (3H, t), 1.55-1.58 (2H, m), 1.64-1.66 (2H, m), 2.56 (1H, s), 2.98-3.02 (1H, m), 3.18 (1H, d), 3.46-3.52 (1H, m), 3.62-3.66 (1H, m), 3.77 (1H, d), 3.96-4.00 (1H, m), 4.17-4.21 (1H, m), 4.53 (1H, s), 6.42 (1H, d), 6.85 (1H, s), 7.49-7.51 (2H, m), 8.20-8.22 (2H, m), 8.53 (1H, s).

LCMS Spectrum: m/z (ESI+) (M+H)+=498; HPLC tR=1.95 min.

mTOR Kinase Assay (Echo): 0.00195 μM

Example 3, 3-cyclopropyl-1-[4-[4-(1-cyclopropylsulfonylcyclopropyl)-6-[(3S)-3-methylmorpholin-4-yl]pyrimidin-2-yl]phenyl]urea, may also be prepared as described below.

To a solution of phenyl N-[4-[4-(1-cyclopropylsulfonylcyclopropyl)-6-[(3S)-3-methylmorpholin-4-yl]pyrimidin-2-yl]phenyl]carbamate (150 mg, 0.28 mmol) in DMF (2 mL) was added triethylamine (0.177 mL, 0.84 mmol) followed by cyclopropylamine (0.097 mL, 1.40 mmol) and the reaction heated at 50° C. for 2 hours. The crude product was purified by preparative HPLC using decreasingly polar mixtures of water (containing 1% NH3) and acetonitrile as eluents, to give the desired material as a white solid (103 mg).

NMR Spectrum: ¹H NMR (400.13 MHz, DMSO-d₆) δ 0.40-0.44 (2H, m), 0.62-0.67 (2H, m), 0.90-0.97 (2H, m), 1.02-1.04 (2H, m), 1.24 (3H, d), 1.56-1.60 (2H, m), 1.65 (2H, d), 2.60 (1H, t), 2.96-3.02 (1H, m), 3.20-3.24 (1H, m), 3.46-3.52 (1H, m), 3.62-3.66 (1H, m), 3.77 (1H, d), 3.96-4.00 (1H, m), 4.19 (1H, d), 4.53 (1H, s), 6.42 (1H, d), 6.85 (1H, s), 7.50 (2H, d), 8.21 (2H, d), 8.53 (1H, s)

LCMS Spectrum: m/z (ESI+)(M+H)+=498; HPLC tR=2.13 min.

The following compounds were prepared in an analogous fashion from phenyl N-[4-[4-(1-cyclopropylsulfonylcyclopropyl)-6-[(3S)-3-methylmorpholin-4-yl]pyrimidin-2-yl]phenyl]carbamate using the appropriate amine.

| Ex- | | | | |
|---|---|---|---|---|
| am- | | | LCMS | Retention |
| ple | Structure | NAME | MH+ | time (min) |
| 3a | 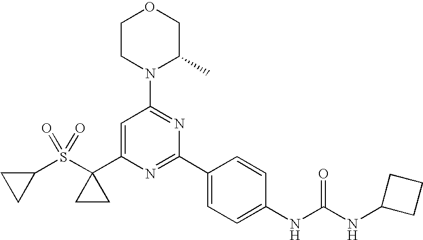 | 3-cyclobutyl-1-[4-[4-(1-cyclopropylsulfonylcyclopropyl)-6-[(3S)-3-methylmorpholin-4-yl]pyrimidin-2-yl]phenyl]urea | 512 | 2.38 |
| 3b | 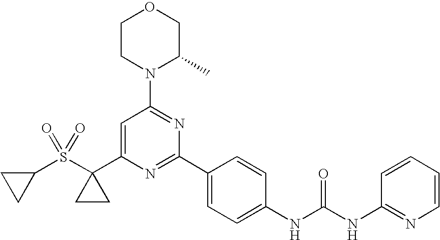 | 1-[4-[4-(1-cyclopropylsulfonylcyclopropyl)-6-[(3S)-3-methylmorpholin-4-yl]pyrimidin-2-yl]phenyl]-3-pyridin-2-yl-urea | 535 | 2.58 |
| 3c | 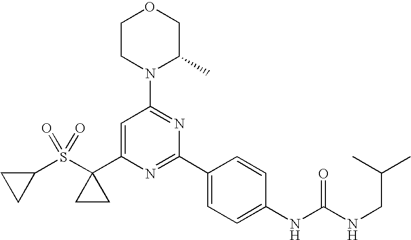 | 1-[4-[4-(1-cyclopropylsulfonylcyclopropyl)-6-[(3S)-3-methylmorpholin-4-yl]pyrimidin-2-yl]phenyl]-3-(2-methylpropyl)urea | 514 | 2.46 |
| 3d | 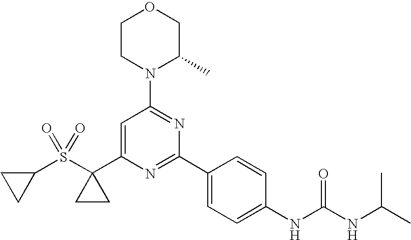 | 1-[4-[4-(1-cyclopropylsulfonylcyclopropyl)-6-[(3S)-3-methylmorpholin-4-yl]pyrimidin-2-yl]phenyl]-3-propan-2-yl-urea | 500 | 2.28 |
| 3e | 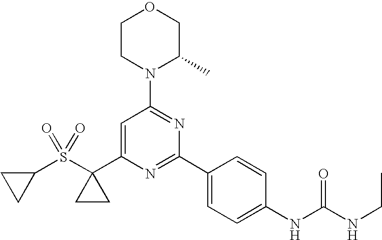 | 3-[4-[4-(1-cyclopropylsulfonylcyclopropyl)-6-[(3S)-3-methylmorpholin-4-yl]pyrimidin-2-yl]phenyl]-1-ethyl-urea | 486 | 2.12 |
| 3f | 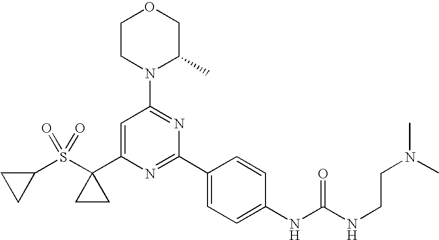 | 1-[4-[4-(1-cyclopropylsulfonylcyclopropyl)-6-[(3S)-3-methylmorpholin-4-yl]pyrimidin-2-yl]phenyl]-3-(2-dimethylaminoethyl)urea | 529 | 2.04 |
| 3g | 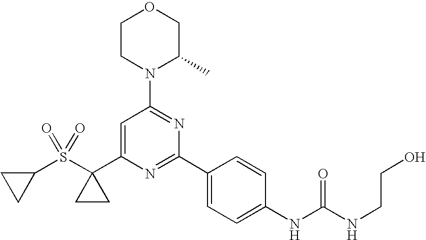 | 1-[4-[4-(1-cyclopropylsulfonylcyclopropyl)-6-[(3S)-3-methylmorpholin-4-yl]pyrimidin-2-yl]phenyl]-3-(2-hydroxyethyl)urea | 502 | 1.79 |
| 3h | 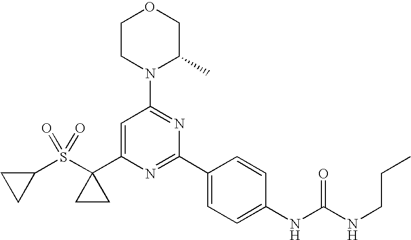 | 3-[4-[4-(1-cyclopropylsulfonylcyclopropyl)-6-[(3S)-3-methylmorpholin-4-yl]pyrimidin-2-yl]phenyl]-1-propyl-urea | 500 | 2.29 |
| 3i | 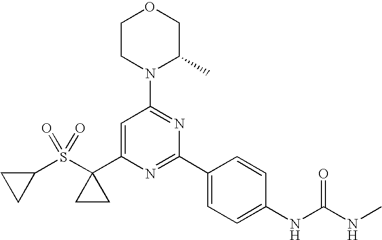 | 1-[4-[4-(1-cyclopropylsulfonylcyclopropyl)-6-[(3S)-3-methylmorpholin-4-yl]pyrimidin-2-yl]phenyl]-3-methyl-urea | 472 | 1.96 |
| 3j | 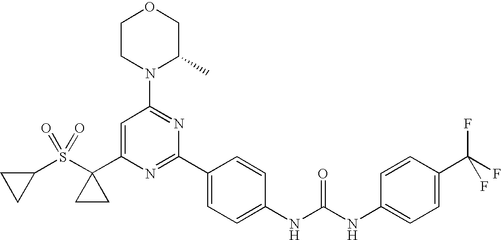 | 1-[4-[4-(1-cyclopropylsulfonylcyclopropyl)-6-[(3S)-3-methylmorpholin-4-yl]pyrimidin-2-yl]phenyl]-3-[4-(trifluoromethyl)phenyl]urea | 602 | 3.00 |
| 3k | 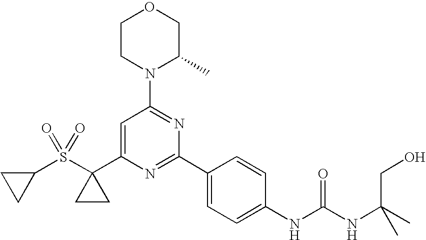 | 1-[4-[4-(1-cyclopropylsulfonylcyclopropyl)-6-[(3S)-3-methylmorpholin-4-yl]pyrimidin-2-yl]phenyl]-3-(1-hydroxy-2-methyl-propan-2-yl)urea | 530 | 2.10 |
| 3l | 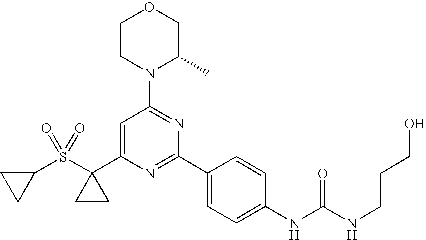 | 1-[4-[4-(1-cyclopropylsulfonylcyclopropyl)-6-[(3S)-3-methylmorpholin-4-yl]pyrimidin-2-yl]phenyl]-3-(3-hydroxypropyl)urea | 516 | 1.83 |
| 3m | 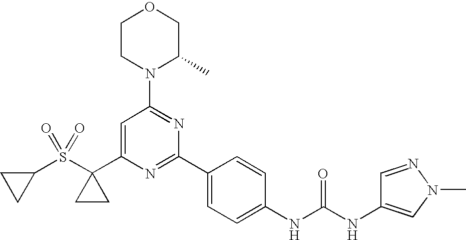 | 1-[4-[4-(1-cyclopropylsulfonylcyclopropyl)-6-[(3S)-3-methylmorpholin-4-yl]pyrimidin-2-yl]phenyl]-3-(1-methylpyrazol-4-yl)urea | 538 | 2.00 |
| 3n | 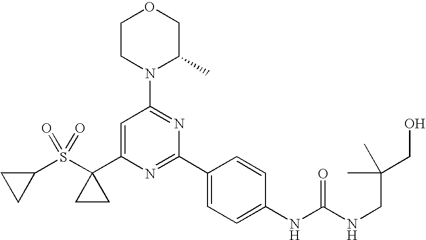 | 1-[4-[4-(1-cyclopropylsulfonylcyclopropyl)-6-[(3S)-3-methylmorpholin-4-yl]pyrimidin-2-yl]phenyl]-3-(3-hydroxy-2,2-dimethylpropyl)urea | 544 | 2.02 |
| 3o | 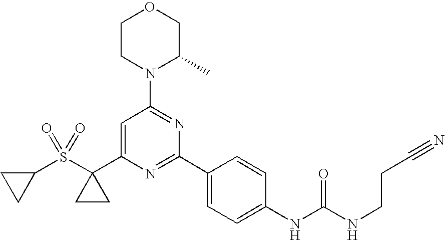 | 3-(2-cyanoethyl)-1-[4-[4-(1-cyclopropylsulfonylcyclopropyl)-6-[(3S)-3-methylmorpholin-4-yl]pyrimidin-2-yl]phenyl]urea | 511 | 1.92 |
| 3p | 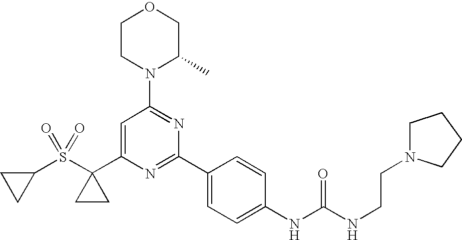 | 1-[4-[4-(1-cyclopropylsulfonylcyclopropyl)-6-[(3S)-3-methylmorpholin-4-yl]pyrimidin-2-yl]phenyl]-3-(2-pyrrolidin-1-ylethyl)urea | 555 | 2.17 |
| 3q | 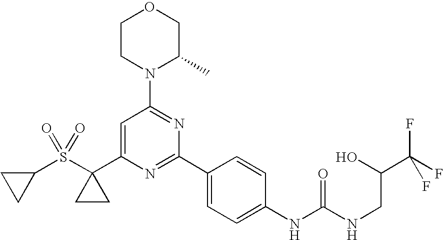 | 1-[4-[4-(1-cyclopropylsulfonylcyclopropyl)-6-[(3S)-3-methylmorpholin-4-yl]pyrimidin-2-yl]phenyl]-3-(3,3,3-trifluoro-2-hydroxypropyl)urea | 570 | 2.01 |
| 3r | 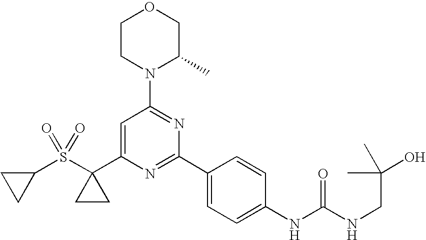 | 1-[4-[4-(1-cyclopropylsulfonylcyclopropyl)-6-[(3S)-3-methylmorpholin-4-yl]pyrimidin-2-yl]phenyl]-3-(2-hydroxy-2-methylpropyl)urea | 530 | 1.91 |
| 3s | 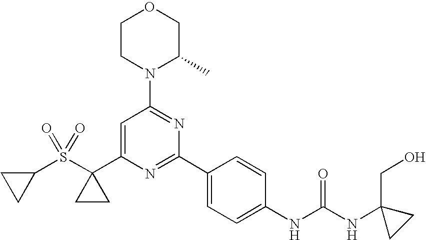 | 1-[4-[4-(1-cyclopropylsulfonylcyclopropyl)-6-[(3S)-3-methylmorpholin-4-yl]pyrimidin-2-yl]phenyl]-3-[1-(hydroxymethyl)cyclopropyl]urea | 528 | 1.91 |
| 3t | 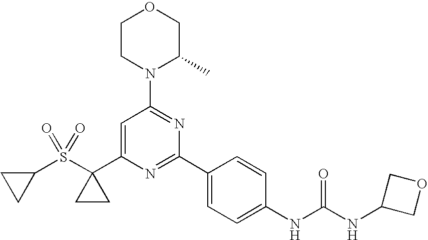 | 1-[4-[4-(1-cyclopropylsulfonylcyclopropyl)-6-[(3S)-3-methylmorpholin-4-yl]pyrimidin-2-yl]phenyl]-3-(oxetan-3-yl)urea | 514 | 1.92 |
| 3u | 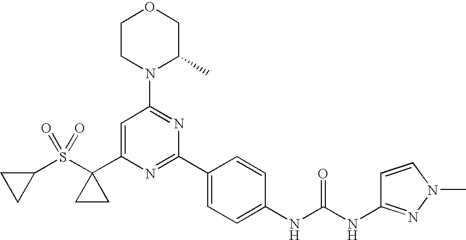 | 1-[4-[4-(1-cyclopropylsulfonylcyclopropyl)-6-[(3S)-3-methylmorpholin-4-yl]pyrimidin-2-yl]phenyl]-3-(1-methylpyrazol-3-yl)urea | 538 | 2.22 |
| 3w | 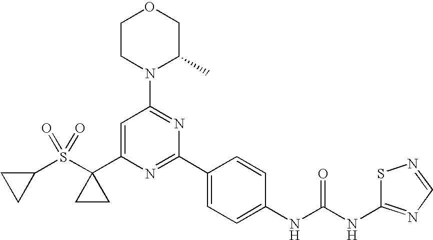 | 1-[4-[4-(1-cyclopropylsulfonylcyclopropyl)-6-[(3S)-3-methylmorpholin-4-yl]pyrimidin-2-yl]phenyl]-3-(1,2,4-thiadiazol-5-yl)urea | 542 | 1.50 |

### EXAMPLE 3a

¹H NMR (400.13 MHz, DMSO-d₆) δ 0.90-0.97 (2H, m), 1.02-1.04 (2H, m), 1.23 (3H, d), 1.56-1.59 (2H, m), 1.63 (2H, d), 1.65 (2H, d), 1.83 (1H, d), 1.86 (1H, t), 2.17-2.25 (2H, m), 2.97-3.01 (1H, m), 3.17-3.24 (1H, m), 3.46-3.52 (1H, m), 3.62-3.66 (1H, m), 3.77 (1H, d), 3.96-3.99 (1H, m), 4.11-4.17 (1H, m), 4.20 (1H, s), 4.53 (1H, d), 6.45 (1H, d), 6.84 (1H, s), 7.46-7.48 (2H, m), 8.20 (2H, d), 8.55 (1H, s).

mTOR Kinase Assay (Echo): 0.00445 μM

### EXAMPLE 3b

¹H NMR (400.13 MHz, DMSO-d₆) δ 0.88-0.98 (2H, m), 0.99-1.06 (2H, m), 1.25 (3H, d), 1.57-1.62 (2H, m), 1.65-1.68 (2H, m), 2.99-3.05 (1H, m), 3.19-3.23 (1H, m), 3.47-3.54 (1H, m), 3.63-3.67 (1H, m), 3.78 (1H, d), 3.97-4.01 (1H, m), 4.21 (1H, d), 4.54 (1H, d), 6.88 (1H, s), 7.02-7.05 (1H, m), 7.56 (1H, d), 7.63 (2H, d), 7.75-7.77 (1H, m), 8.29-8.31 (3H, m), 9.44 (1H, d), 10.58 (1H, s).

mTOR Kinase Assay (Echo): 0.00385 μM

### EXAMPLE 3c

¹H NMR (400.13 MHz, DMSO-d₆) δ 0.88-0.90 (6H, m), 0.94 (2H, t), 1.00-1.06 (2H, m), 1.24 (3H, d), 1.55-1.58 (2H, m), 1.64-1.66 (2H, m), 1.67-1.74 (1H, m), 2.94 (2H, t), 2.98-3.02 (1H, m), 3.20-3.24 (1H, m), 3.46-3.52 (1H, m), 3.62-3.66 (1H, m), 3.77 (1H, d), 3.96-4.00 (1H, m), 4.19 (1H, d), 4.53 (1H, s), 6.23 (1H, t), 6.84 (1H, s), 7.47-7.50 (2H, m), 8.20-8.22 (2H, m), 8.63 (1H, s).

mTOR Kinase Assay (Echo): 0.0124 μM

### EXAMPLE 3d

¹H NMR (400.13 MHz, DMSO-d₆) δ 0.91-0.97 (2H, m), 1.00-1.05 (2H, m), 1.11 (6H, d), 1.23 (3H, d), 1.55-1.58 (2H, m), 1.64-1.66 (2H, m), 2.97-3.03 (1H, m), 3.17-3.24 (1H, m), 3.46-3.52 (1H, m), 3.62-3.66 (1H, m), 3.76 (2H, d), 3.96-4.00 (1H, m), 4.19 (1H, d), 4.53 (1H, s), 6.05 (1H, d), 6.84 (1H, s), 7.46-7.49 (2H, m), 8.19-8.22 (2H, m), 8.52 (1H, s).

mTOR Kinase Assay (Echo): 0.0135 μM

### EXAMPLE 3e

¹H NMR (400.13 MHz, DMSO-d₆) δ 0.92-0.94 (2H, t), 1.02-1.04 (2H, m), 1.07 (3H, t), 1.23 (3H, d), 1.58 (2H, d), 1.64-1.66 (2H, m), 2.98-3.02 (1H, m), 3.17-3.18 (1H, m), 3.14-3.24 (2H, m), 3.47-3.52 (1H, m), 3.62-3.66 (1H, m), 3.77 (1H, d), 3.96-4.00 (1H, m), 4.19 (1H, d), 4.53 (1H, s), 6.15 (1H, t), 6.84 (1H, s), 7.47-7.51 (2H, m), 8.19-8.22 (2H, m), 8.65 (1H, s).

mTOR Kinase Assay (Echo): 0.00166 μM

### EXAMPLE 3f

¹H NMR (400.13 MHz, DMSO-d₆) δ 0.91-0.97 (2H, m), 0.98-1.05 (2H, m), 1.23 (3H, d), 1.55-1.58 (2H, m), 1.64-1.66 (2H, m), 2.18 (6H, s), 2.34 (2H, t), 2.97-3.03 (1H, m), 3.19 (3H, q), 3.46-3.52 (1H, m), 3.62-3.66 (1H, m), 3.77 (1H, d), 3.96-4.00 (1H, m), 4.19 (1H, d), 4.53 (1H, s), 6.15 (1H, t), 6.84 (1H, s), 7.47-7.50 (2H, m), 8.19-8.22 (2H, m), 8.88 (1H, s).

mTOR Kinase Assay (Echo): 0.0214 μM

### EXAMPLE 3g

¹H NMR (400.13 MHz, DMSO-d₆) δ 0.91-0.97 (2H, m), 1.00-1.05 (2H, m), 1.23 (3H, d), 1.55-1.58 (2H, m), 1.64-1.66 (2H, m), 2.97-3.03 (1H, m), 3.16-3.21 (3H, m), 3.44-3.52 (3H, m), 3.62-3.66 (1H, m), 3.77 (1H, d), 3.96-4.00 (1H, m), 4.17-4.21 (1H, m), 4.53 (1H, s), 4.72 (1H, t), 6.25 (1H, t), 6.84 (1H, s), 7.47-7.50 (2H, m), 8.20-8.22 (2H, m), 8.79 (1H, s).

mTOR Kinase Assay (Echo): 0.00134 μM

### EXAMPLE 3h

¹H NMR (400.13 MHz, DMSO-d₆) δ 0.89 (3H, t), 0.91-0.95 (2H, m), 1.02-1.04 (2H, m), 1.23 (3H, d), 1.41-1.50 (2H, m), 1.56-1.60 (2H, m), 1.64-1.66 (2H, m), 2.98-3.02 (1H, m), 3.04-3.09 (2H, m), 3.20-3.24 (1H, m), 3.47-3.52 (1H, m), 3.62-3.66 (1H, m), 3.77 (1H, d), 3.96-4.00 (1H, m), 4.19 (1H, d), 4.53 (1H, s), 6.20 (1H, t), 6.84 (1H, s), 7.47-7.50 (2H, m), 8.20-8.22 (2H, m), 8.64 (1H, s).

mTOR Kinase Assay (Echo): 0.0165 μM

### EXAMPLE 3i

¹H NMR (400.13 MHz, DMSO-d₆) δ 0.92-0.95 (2H, m), 1.02-1.05 (2H, m), 1.23 (3H, d), 1.56-1.60 (2H, m), 1.63-1.66 (2H, m), 2.66 (3H, d), 2.98-3.02 (1H, m), 3.17-3.24 (1H, m), 3.46-3.52 (1H, m), 3.62-3.66 (1H, m), 3.77 (1H, d), 3.96-4.00 (1H, m), 4.19 (1H, d), 4.53 (1H, s), 6.05 (1H, t), 6.84 (1H, s), 7.48-7.51 (2H, m), 8.19-8.22 (2H, m), 8.73 (1H, s).

mTOR Kinase Assay (Echo): 0.000932 μM

### EXAMPLE 3j

¹H NMR (400.13 MHz, DMSO-d₆) δ 0.92-0.98 (2H, m), 1.01-1.05 (2H, m), 1.25 (3H, d), 1.57-1.62 (1H, m), 1.61 (1H, d), 1.65-1.68 (2H, m), 2.97-3.04 (1H, m), 3.22-3.26 (1H, m), 3.47-3.53 (1H, m), 3.63-3.67 (1H, m), 3.78 (1H, d), 3.97-4.00 (1H, m), 4.21 (1H, d), 4.55 (1H, s), 6.87 (1H, s), 7.57-7.59 (2H, m), 7.63-7.70 (4H, m), 8.29 (2H, d), 9.04 (1H, s), 9.13 (1H, s).

mTOR Kinase Assay (Echo): 0.00422 μM

### EXAMPLE 3k

¹H NMR (400.13 MHz, DMSO-d₆) δ 0.94 (2H, t), 1.04 (2H, d), 1.23 (6H, d), 1.24 (3H, d), 1.56-1.59 (2H, m), 1.64-1.66 (2H, m), 2.98-3.02 (1H, m), 3.18 (1H, d), 3.39 (2H, d), 3.46-3.52 (1H, m), 3.62-3.66 (1H, m), 3.77 (1H, d), 3.96-4.00 (1H, m), 4.19 (1H, d), 4.52 (1H, s), 4.95 (1H, t), 6.00 (1H, s), 6.84 (1H, s), 7.43-7.47 (2H, m), 8.19-8.21 (2H, m), 8.73 (1H, s).

mTOR Kinase Assay (Echo): 0.00227 μM

### EXAMPLE 3l

¹H NMR (400.13 MHz, DMSO-d₆) δ 0.91-0.94 (2H, m), 1.01-1.05 (2H, m), 1.23 (3H, d), 1.55-1.58 (2H, m), 1.60-1.61 (2H, m), 1.65 (1H, m), 2.98-3.02 (1H, m), 3.17 (1H, d), 3.18-3.23 (2H, m), 3.45-3.49 (3H, m), 3.50 (1H, d), 3.62-3.66 (1H, m), 3.77 (1H, d), 3.96-4.00 (1H, m), 4.17 (1H, s), 4.47 (1H, t), 4.53 (1H, s), 6.20 (1H, t), 6.84 (1H, s), 7.48-7.50 (2H, m), 8.21 (2H, d), 8.70 (1H, s).

mTOR Kinase Assay (Echo): 0.00257 μM

### EXAMPLE 3m

¹H NMR (400.13 MHz, DMSO-d₆) δ 0.93-0.95 (2H, m), 1.02-1.05 (2H, m), 1.24 (3H, d), 1.57-1.60 (2H, m), 1.65-1.67 (2H, m), 2.98-3.02 (1H, m), 3.18 (1H, t), 3.46-3.50 (1H, m), 3.63-3.66 (1H, m), 3.76 (1H, s), 3.79 (3H, s), 3.96-4.00 (1H, m), 4.18 (1H, s), 4.53 (1H, s), 6.86 (1H, s), 7.38-7.38 (1H, m), 7.55 (2H, t), 7.76 (1H, s), 8.25 (2H, d), 8.38 (1H, s), 8.83 (1H, s).

mTOR Kinase Assay (Echo): 0.000497 μM

### EXAMPLE 3n

¹H NMR (400.13 MHz, DMSO-d₆) δ 0.81 (6H, s), 0.91-0.95 (2H, m), 1.01-1.05 (2H, m), 1.23 (3H, d), 1.56-1.60 (3H, m), 1.63-1.66 (2H, m), 2.98-3.02 (2H, m), 3.15 (2H, d), 3.21 (1H, dt), 3.49 (1H, dt), 3.64 (1H, dd), 3.77 (1H, d), 4.19 (1H, d), 4.53 (1H, s), 4.61 (1H, t), 6.26 (1H, t), 6.84 (1H, s), 7.48 (2H, d), 8.21 (2H, d), 8.78 (1H, s).

mTOR Kinase Assay (Echo): 0.0396 μM

### EXAMPLE 3o

¹H NMR (400.13 MHz, DMSO-d₆) δ 0.90-0.95 (2H, m), 1.00-1.06 (2H, m), 1.24 (3H, d), 1.56-1.60 (2H, m), 1.63-1.66 (2H, m), 2.70 (2H, t), 2.96-3.03 (1H, m), 3.17-3.25 (1H, m), 3.35-3.39 (2H, m), 3.49 (1H, dt), 3.64 (1H, dd), 3.77 (1H, d), 3.98 (1H, dd), 4.19 (1H, d), 4.53 (1H, s), 6.52 (1H, t), 6.85 (1H, s), 7.51 (2H, d), 8.22 (2H, d), 8.91 (1H, s).

mTOR Kinase Assay (Echo): 0.00596 μM

### EXAMPLE 3p

¹H NMR (400.13 MHz, DMSO-d₆) δ 0.93-0.94 (2H, m), 1.02-1.04 (2H, m), 1.23 (3H, d), 1.56-1.58 (2H, m), 1.64-1.67 (2H, m), 1.72 (8H, m), 2.94-3.02 (1H, m), 3.18-3.24 (5H, m), 3.49 (1H, t), 3.64 (1H, d), 3.77 (1H, d), 3.97 (1H, d), 4.19 (1H, d), 4.53 (1H, s), 6.22 (1H, s), 6.84 (1H, s), 7.49 (2H, d), 8.21 (2H, d), 8.88 (1H, s).

mTOR Kinase Assay (Echo): 0.193 μM

### EXAMPLE 3q

¹H NMR (400.13 MHz, DMSO-d₆) δ 0.91-0.95 (2H, m), 1.00-1.06 (2H, m), 1.23 (3H, d), 1.56-1.60 (2H, m), 1.63-1.66 (2H, m), 2.96-3.03 (1H, m), 3.14-3.25 (2H, m), 3.46-3.55 (2H, m), 3.64 (1H, dd), 3.77 (1H, d), 3.98 (1H, dd), 4.07-4.08 (1H, m), 4.19 (1H, d), 4.52 (1H, s), 6.42 (1H, t), 6.49 (1H, s), 6.85 (1H, s), 7.50 (2H, d), 8.22 (2H, d), 8.95 (1H, s).

mTOR Kinase Assay (Echo): 0.00839 μM

### EXAMPLE 3r

¹H NMR (400.13 MHz, DMSO-d₆) δ 0.91-0.95 (2H, m), 1.00-1.06 (2H, m), 1.10 (6H, s), 1.23 (3H, d), 1.56-1.60 (2H, m), 1.61-1.66 (2H, m), 2.97-3.02 (1H, m), 3.06 (2H, d), 3.22 (1H, dd), 3.49 (1H, dt), 3.64 (1H, dd), 3.77 (1H, d), 3.98 (1H, dd), 4.19 (1H, d), 4.52 (1H, s), 4.54 (1H, s), 6.24 (1H, t), 6.84 (1H, s), 7.48 (2H, d), 8.21 (2H, d), 8.88 (1H, s).

mTOR Kinase Assay (Echo): 0.0488 μM

### EXAMPLE 3s

¹H NMR (400.13 MHz, DMSO-d₆) δ 0.63-0.67 (2H, m), 0.69-0.73 (2H, m), 0.91-0.95 (2H, m), 1.02-1.04 (2H, m), 1.23 (3H, d), 1.56-1.60 (2H, m), 1.63-1.66 (2H, m), 2.97-3.03 (1H, m), 3.17-3.25 (2H, m), 3.44 (1H, d), 3.46-3.52 (1H, m), 3.64 (1H, dd), 3.77 (1H, d), 3.98 (1H, dd), 4.19 (1H, d), 4.52 (1H, s), 4.83 (1H, s), 6.56 (1H, s), 6.85 (1H, s), 7.47 (2H, d), 8.21 (2H, d), 8.67 (1H, s).

mTOR Kinase Assay (Echo): 0.0263 μM

### EXAMPLE 3t

¹H NMR (400.13 MHz, DMSO-d₆) δ 0.91-0.95 (2H, m), 1.00-1.07 (2H, m), 1.23 (3H, d), 1.56-1.61 (2H, m), 1.63-1.66 (2H, m), 2.96-3.02 (1H, m), 3.21 (1H, dt), 3.49 (1H, dt), 3.64 (1H, dd), 3.77 (1H, d), 3.98 (1H, dd), 4.19 (1H, d), 4.45 (2H, t), 4.52 (1H, s), 4.72-4.83 (3H, m), 6.85 (1H, s), 6.95 (1H, d), 7.49 (2H, d), 8.22 (2H, d), 8.78 (1H, s).

mTOR Kinase Assay (Echo): 0.00479 μM

### EXAMPLE 3u

¹H NMR (400.13 MHz, DMSO-d₆) δ 0.91-0.95 (2H, m), 1.00-1.07 (2H, m), 1.23 (3H, d), 1.56-1.61 (2H, m), 1.63-1.66 (2H, m), 2.96-3.02 (1H, m), 3.21 (1H, dt), 3.49 (1H, dt), 3.64 (1H, dd), 3.77 (1H, d), 3.98 (1H, dd), 4.19 (1H, d), 4.45 (2H, t), 4.52 (1H, s), 4.72-4.83 (3H, m), 6.85 (1H, s), 6.95 (1H, d), 7.49 (2H, d), 8.22 (2H, d), 8.78 (1H, s).

mTOR Kinase Assay (Echo): 0.000604 μM

### EXAMPLE 3v

¹H NMR (400.13 MHz, DMSO-d₆) δ 0.94-0.96 (2H, m), 1.03-1.05 (2H, m), 1.24 (3H, d), 1.57-1.59 (2H, m), 1.64-1.66 (2H, m), 2.98-3.03 (1H, m), 3.19-3.25 (1H, m), 3.50 (1H, t), 3.65 (1H, d), 3.74 (3H, s), 3.77 (1H, d), 3.98 (1H, d), 4.21 (1H, d), 4.54 (1H, s), 6.24 (1H, d), 6.86 (1H, s), 7.54-7.55 (1H, m), 7.56 (2H, d), 8.26 (2H, d), 8.92 (1H, s), 9.17 (1H, s).

### EXAMPLE 3w

¹H NMR (400.13 MHz, DMSO-d₆) δ 0.92-0.96 (2H, m), 1.02-1.06 (2H, m), 1.25 (3H, d), 1.58-1.60 (2H, m), 1.66-1.67 (2H, m), 2.98-3.05 (1H, m), 3.20-3.23 (1H, m), 3.50 (1H, t), 3.65 (1H, d), 3.78 (1H, d), 3.99 (1H, d), 4.21 (1H, d), 4.55 (1H, s), 6.89 (1H, s), 7.63 (2H, d), 8.31-8.36 (3H, m), 9.43 (1H, s).

mTOR Kinase Assay (Echo): 0.00089 μM

The preparation of phenyl N-[4-[4-(1-cyclopropylsulfonylcyclopropyl)-6-[(3S)-3-methylmorpholin-4-yl]pyrimidin-2-yl]phenyl]carbamate is described below:

### Phenyl N-[4-[4-(1-cyclopropylsulfonylcyclopropyl)-6-[(3S)-3-methylmorpholin-4-yl]pyrimidin-2-yl]phenyl]carbamate

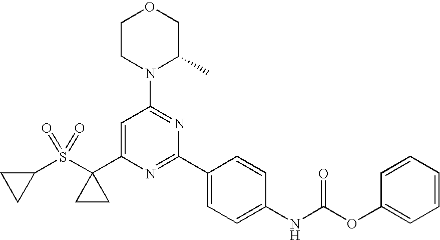

Phenyl chloroformate (0.729 mL, 5.79 mmol) was added dropwise to 4-[4-(1-cyclopropylsulfonylcyclopropyl)-6-[(3S)-3-methylmorpholin-4-yl]pyrimidin-2-yl]aniline (2.40 g, 5.79 mmol) and sodium bicarbonate (0.730 g, 8.68 mmol) in dioxane (45 mL) under nitrogen. The resulting suspension was stirred at 20° C. for 2 hours. The reaction mixture was evaporated to dryness and redissolved in ethyl acetate (200 mL) and washed with water (200 mL). The organic layer was dried (MgSO₄), filtered and evaporated to afford the desired material as a white solid (3.03 g).

NMR Spectrum: ¹H NMR (400.13 MHz, DMSO-d6) δ 0.92-0.95 (2H, m), 1.03-1.05 (2H, m), 1.25 (3H, d), 1.57-1.61 (1H, m), 1.61 (1H, d), 1.65-1.68 (2H, m), 2.99-3.03 (1H, m), 3.46-3.47 (1H, m), 3.49-3.53 (1H, m), 3.63-3.66 (1H, m), 3.77 (1H, d), 3.96-4.00 (1H, m), 4.22 (1H, d), 4.56 (1H, s), 6.89 (1H, s), 7.26 (1H, t), 7.24-7.30 (2H, m), 7.43-7.47 (2H, m), 7.60-7.65 (2H, m), 8.29-8.32 (2H, m), 10.43 (1H, s)

LCMS Spectrum: m/z (ESI+) (M+H)+=535; HPLC tR=2.84 min.

### 4-[4-(1-Cyclopropylsulfonylcyclopropyl)-6-[(3S)-3-methylmorpholin-4-yl]pyrimidin-2-yl]aniline

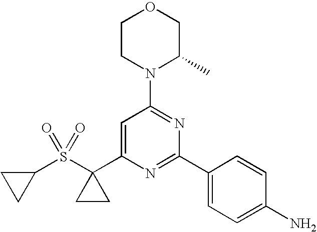

Dichlorobis(triphenylphosphine)palladium(II) (0.524 g, 0.75 mmol) was added to a degassed solution of 2-chloro-4-(1-cyclopropylsulfonylcyclopropyl)-6-[(3S)-3-methylmorpholin-4-yl]pyrimidine (2.67 g, 7.46 mmol), 4-(4,4,5,5-tetramethyl-1,3,2-dioxaborolan-2-yl)aniline (2.043 g, 9.33 mmol) and sodium carbonate (18.65 mL, 37.31 mmol) in 18% DMF in 7:3:2 DME:Water:Ethanol (20 mL). The resulting solution was stirred at 85° C. for 1 hour. The reaction mixture was concentrated and diluted with DCM (150 mL), and washed with water (100 mL) and brine (100 mL). The organic layer was dried (MgSO₄), filtered and evaporated to afford crude product. The crude product was purified by flash silica chromatography, elution gradient 0 to 2.5% methanol in DCM, to give the desired material as a brown solid (2.40 g).

NMR Spectrum: ¹H NMR (400.13 MHz, DMSO-d6) δ 0.90-0.98 (2H, m), 0.98-1.05 (2H, m), 1.22 (3H, d), 1.52-1.59 (2H, m), 1.62-1.64 (2H, m), 2.95-3.02 (1H, m), 3.14-3.22 (1H, m), 3.45-3.51 (1H, m), 3.61-3.65 (1H, m), 3.76 (1H, d), 3.95-3.98 (1H, m), 4.14-4.17 (1H, m), 4.48-4.51 (1H, m), 5.53 (2H, d), 6.60 (2H, d), 6.75 (1H, s), 8.03-8.06 (2H, m).

LCMS Spectrum: m/z (ESI+) (M+H)+=415; HPLC tR=2.13 min.

### 2-Chloro-4-(1-cyclopropylsulfonylcyclopropyl)-6-[(3S)-3-methylmorpholin-4-yl]pyrimidine

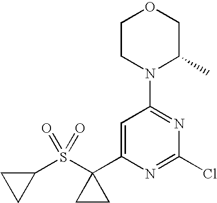

5N Sodium hydroxide solution (1.74 mL, 8.68 mmol) was added to tetrabutylammonium bromide (0.140 g, 0.43 mmol), 1,2-dibromoethane (0.374 mL, 4.34 mmol) and 2-chloro-4-(cyclopropylsulfonylmethyl)-6-[(3S)-3-methylmorpholin-4-yl]pyrimidine (1.44 g, 4.34 mmol) in DCM (20 mL). The resulting mixture was stirred at 40° C. for 2 hours. Additional solid sodium hydroxide (4 g, 0.1 mol) was added directly to the reaction and stirred at 40° C. for a further 1 hour. The reaction mixture was diluted with DCM (20 mL) and washed with water (50 mL). The organic layer was dried (MgSO₄), filtered and evaporated to afford the desired material (1.68 g).

LCMS Spectrum: m/z (ESI+) (M+H)+=358; HPLC tR=1.87 min.

### 2-Chloro-4-(cyclopropylsulfonylmethyl)-6-[(3S)-3-methylmorpholin-4-yl]pyrimidine

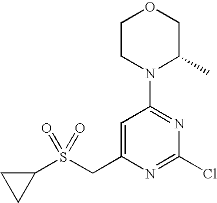

Cyclopropanesulfinic acid, sodium salt (381 mg, 2.97 mmol) was added in one portion to 2-chloro-4-(iodomethyl)-6-[(3S)-3-methylmorpholin-4-yl]pyrimidine (700 mg, 1.98 mmol) in acetonitrile (20 mL) at RT. The resulting suspension was stirred at 90° C. for 3 hours. The reaction mixture was evaporated to dryness and redissolved in DCM (50 mL), and washed with water (50 mL). The organic layer was dried (MgSO₄), filtered and evaporated to afford crude product. The crude product was purified by flash silica chromatography, elution gradient 0 to 40% ethyl acetate in DCM, to gve the desired material as a white solid (458 mg).

NMR Spectrum: ¹H NMR (400.132 MHz, DMSO) δ 0.95-0.98 (2H, m), 1.02-1.06 (2H, m), 1.18-1.23 (3H, m), 2.77-2.83 (1H, m), 3.19-3.25 (1H, m), 3.42-3.49 (1H, m), 3.58-3.62 (1H, m), 3.73 (1H, d), 3.92-3.96 (2H, m), 4.30 (1H, s), 4.48 (2H, s), 6.92 (1H, s).

LCMS Spectrum: m/z (ESI+) (M+H)+=332; HPLC tR=1.68 min.

The preparation of 2-chloro-4-(iodomethyl)-6-[(3S)-3-methylmorpholin-4-yl]pyrimidine was described earlier.

### EXAMPLE 4

### 3-Methyl-1-[4-[4-[(3S)-3-methylmorpholin-4-yl]-6-(1-methylsulfonylcyclopentyl)pyrimidin-2-yl]phenyl]urea

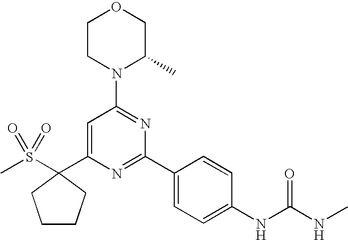

Triethylamine (0.15 mL, 1.1 mmol) was added to a solution of phenyl N-[4-[4-[(3S)-3-methylmorpholin-4-yl]-6-(1-methylsulfonylcyclopentyl)pyrimidin-2-yl]phenyl]carbamate (200 mg, 0.37 mmol) and methylamine (2M in THF, 1.48 mmol) in NMP (2 mL). The reaction was heated at 80° C. for 2 hours the purified by prep HPLC, using a mixture of water (containing 1% NH3) and acetonitrile as eluents, to give the desired material as a solid (126 mg).

NMR Spectrum: ¹H NMR (400.132 MHz, DMSO-d₆) δ 1.21 (3H, d), 1.52-1.62 (2H, m), 1.75-1.90 (2H, m), 2.38-2.50 (2H, m), 2.65 (3H, d), 2.65-2.78 (2H, m), 2.89 (3H, s), 3.20 (1H, dd), 3.50 (1H, dd), 3.64 (1H, d), 3.75 (1H, d), 3.95 (1H, dd), 4.25 (1H, d), 4.55 (1H, s), 6.05 (1H, q), 6.79 (1H, s), 7.50 (2H, d), 8.22 (2H, d), 8.72 (1H, s).

LCMS Spectrum: m/z (ESI+)(M+H)+=474; HPLC tR=1.96 min

mTOR Kinase Assay (Echo): 0.000699 μM

The following compounds were prepared in an analogous fashion from phenyl N-[4-[4-[(3S)-3-methylmorpholin-4-yl]-6-(1-methylsulfonylcyclopentyl)pyrimidin-2-yl]phenyl]carbamate and the appropriate amine.

| | | | LCMS | Retention |
|---|---|---|---|---|
| Example | Structure | NAME | MH+ | time (min) |
| 4a | 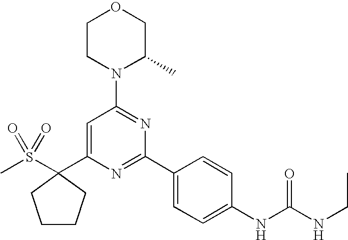 | 3-ethyl-1-[4-[4-[(3S)-3-methylmorpholin-4-yl]-6-(1-methylsulfonylcyclopentyl)pyrimidin-2-yl]phenyl]urea | 488 | 2.13 |
| 4b | 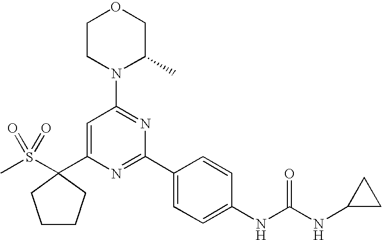 | 3-cyclopropyl-1-[4-[4-(3S)-3-methylmorpholin-4-yl]-6-(1-methylsulfonylcyclopentyl)pyrimidin-2-yl]phenyl]urea | 500 | 2.13 |
| 4c | 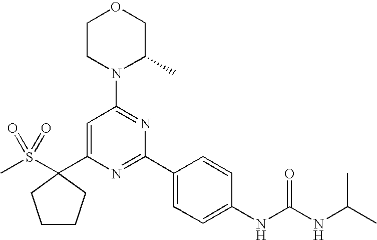 | 1-[4-[4-[(3S)-3-methylmorpholin-4-yl]-6-(1-methylsulfonylcyclopentyl)pyrimidin-2-yl]phenyl]-3-propan-2-yl-urea | 502 | 2.29 |
| 4d | 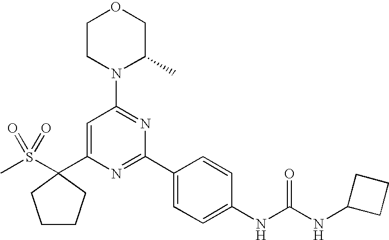 | 3-cyclobutyl-1-[4-[4-[(3S)-3-methylmorpholin-4-yl]-6-(1-methylsulfonylcyclopentyl)pyrimidin-2-yl]phenyl]urea | 514 | 2.35 |
| 4e | 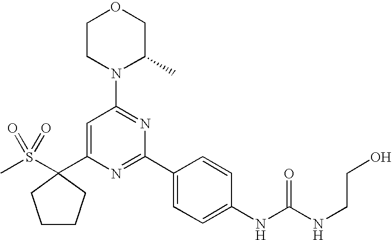 | 3-(2-hydroxyethyl)-1-[4-[4-[(3S)-3-methylmorpholin-4-yl]-6-(1-methylsulfonylcyclopentyl)pyrimidin-2-yl]phenyl]urea | 504 | 1.80 |
| 4f | 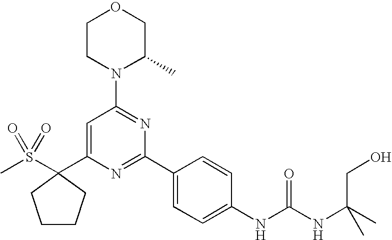 | 3-(1-hydroxy-2-methyl-propan-2-yl)-1-[4-[4-[(3S)-3-methylmorpholin-4-yl]-6-(1-methylsulfonylcyclopentyl)pyrimidin-2-yl]phenyl]urea | 532 | 2.09 |
| 4g | 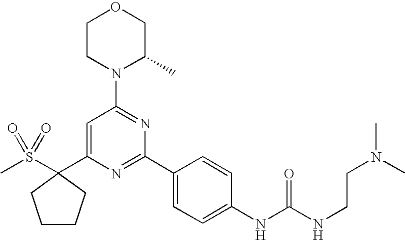 | 3-(2-dimethylaminoethyl)-1-[4-[4-[(3S)-3-methylmorpholin-4-yl]-6-(1-methylsulfonylcyclopentyl)pyrimidin-2-yl]phenyl]urea | 531 | 2.07 |
| 4h | 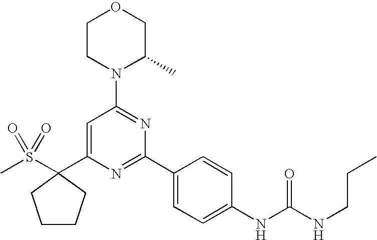 | 1-[4-[4-[(3S)-3-methylmorpholin-4-yl]-6-(1-methylsulfonylcyclopentyl)pyrimidin-2-yl]phenyl]-3-propyl-urea | 502 | 2.28 |
| 4i | 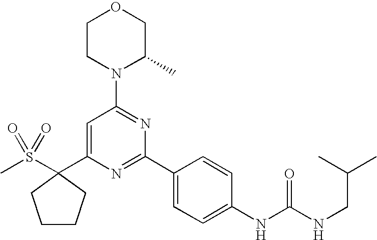 | 1-[4-[4-[(3S)-3-methylmorpholin-4-yl]-6-(1-methylsulfonylcyclopentyl)pyrimidin-2-yl]phenyl]-3-(2-methylpropyl)urea | 517 | 2.45 |
| 4j | 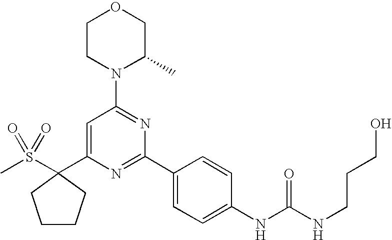 | 3-(3-hydroxypropyl)-1-[4-[4-[(3S)-3-methylmorpholin-4-yl]-6-(1-methylsulfonylcyclopentyl)pyrimidin-2-yl]phenyl]urea | 518 | 1.84 |
| 4k | 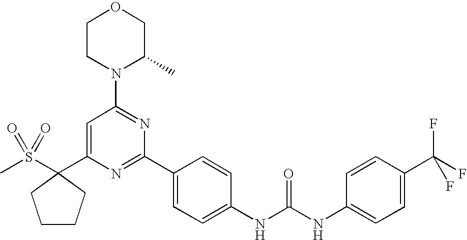 | 1-[4-[4-[(3S)-3-methylmorpholin-4-yl]-6-(1-methylsulfonylcyclopentyl)pyrimidin-2-yl]phenyl]-3-[4-(trifluoromethyl)phenyl]urea | 604 | 2.95 |
| 4l | 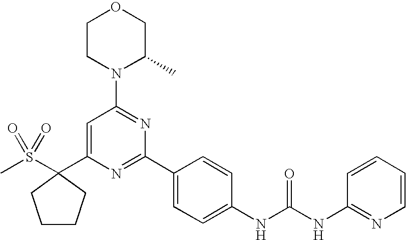 | 1-[4-[4-[(3S)-3-methylmorpholin-4-yl]-6-(1-methylsulfonylcyclopentyl)pyrimidin-2-yl]phenyl]-3-pyridin-2-yl-urea | 537 | 2.55 |
| 4m | 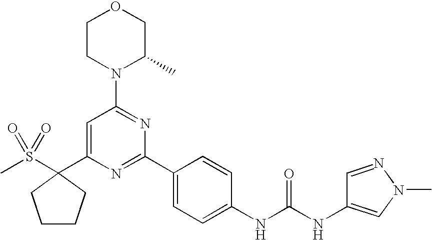 | 1-[4-[4-[(3S)-3-methylmorpholin-4-yl]-6-(1-methylsulfonylcyclopentyl)pyrimidin-2-yl]phenyl]-3-(1-methylpyrazol-4-yl)urea | 540 | 1.99 |
| 4n | 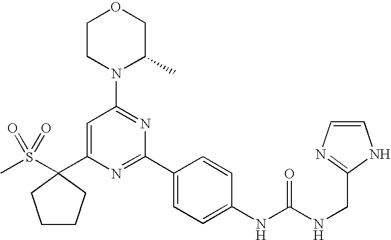 | 3-(1H-imidazol-2-ylmethyl)-1-[4-[4-[(3S)-3-methylmorpholin-4-yl]-6-(1-methylsulfonylcyclopentyl)pyrimidin-2-yl]phenyl]urea | 540 | 1.92 |

### EXAMPLE 4a

¹H NMR (400.132 MHz, DMSO) δ 1.05 (3H, t), 1.21 (3H, d), 1.50-1.62 (2H, m), 1.78-1.85 (2H, m), 2.35-2.45 (2H, m), 2.65-2.75 (2H, m), 2.89 (3H, s), 3.12 (2H, q), 3.18 (1H, dd), 3.50 (1H, dd), 3.65 (1H, dd), 3.75 (1H, d), 3.96 (1H, dd), 4.24 (1H, d), 4.55 (1H, s), 6.15 (1H, t), 6.78 (1H, s), 7.48 (2H, d), 8.22 (2H, d), 8.65 (1H, s).

mTOR Kinase Assay (Echo): 0.00216 μM

### EXAMPLE 4b

¹H NMR (400.132 MHz, DMSO) δ 0.41 (2H, q), 0.62 (2H, q), 1.21 (3H, d), 1.51-1.61 (2H, m), 1.75-1.86 (2H, m), 2.35-2.45 (2H, m), 2.55 (1H, m), 2.65-2.85 (2H, m), 2.89 (3H, s), 3.20 (1H, dd), 3.50 (1H, dd), 3.65 (1H, dd), 3.75 (1H, d), 3.97 (1H, dd), 4.25 (1H, d), 4.55 (1H, s), 6.41 (1H, d), 6.78 (1H, s), 7.50 (2H, d), 8.25 (2H, d), 8.55 (1H, s).

mTOR Kinase Assay (Echo): 0.00203 μM

### EXAMPLE 4c

¹H NMR (400.132 MHz, DMSO) δ 1.11 (6H, d), 1.21 (3H, d), 1.50-1.62 (2H, m), 1.75-1.85 (2H, m), 2.38-2.50 (3H, m), 2.65-2.80 (2H, m), 2.89 (3H, s), 3.20 (1H, dd), 3.50 (1H, dd), 3.65 (1H, dd), 3.75 (1H, dd), 3.99 (1H, dd), 4.23 (1H, d), 4.55 (1H, s), 6.05 (1H, d), 6.79 (1H, s), 7.48 (2H, d), 8.22 (2H, d), 8.54 (1H, s).

mTOR Kinase Assay (Echo): 0.0169 μM

### EXAMPLE 4d

¹H NMR (400.132 MHz, DMSO) δ 1.21 (3H, d), 1.50-1.65 (4H, m), 1.75-1.90 (4H, m), 2.15-2.20 (2H, m), 2.35-2.50 (2H, m), 2.65-2.80 (2H, m), 2.88 (3H, s), 3.20 (1H, dd), 3.50 (1H, dd), 3.65 (1H, dd), 3.75 (1H, dd), 3.95 (1H, dd), 4.15 (1H, q), 4.22 (1H, d), 4.55 (1H, s), 6.42 (1H, d), 6.79 (1H, s), 7.45 (2H, d), 8.22 (2H, d), 8.55 (1H, s).

mTOR Kinase Assay (Echo): 0.01 μM

### EXAMPLE 4e

¹H NMR (400.132 MHz, DMSO) δ 1.21 (3H, d), 1.50-1.62 (2H, m), 1.78-1.85 (2H, m), 2.38-2.50 (2H, m), 2.65-2.80 (2H, m), 2.89 (3H, s), 3.15-3.22 (3H, m), 3.40-3.50 (3H, m), 3.65 (1H, dd), 3.75 (1H, d), 3.96 (1H, dd), 4.22 (1H, d), 4.55 (1H, s), 4.71 (1H, t), 6.22 (1H, t), 6.78 (1H, s), 7.45 (2H, d), 8.22 (2H, d), 8.80 (1H, s).

mTOR Kinase Assay (Echo): 0.00119 μM

### EXAMPLE 4f

¹H NMR (400.132 MHz, DMSO) δ 1.21 (3H, d), 1.50-1.62 (2H, m), 1.75-1.85 (2H, m), 2.38-2.50 (2H, m), 2.65-2.80 (2H, m), 2.89 (3H, s), 3.20 (1H, dd), 3.35-3.40 (2H, m), 3.50 (1H, dd), 3.65 (1H, dd), 3.75 (1H, d), 3.95 (1H, dd), 4.22 (1H, d), 4.55 (1H, s), 4.95 (1H, t), 6.0 (1H, s), 6.78 (1H, s), 7.45 (2H, d), 8.22 (2H, d), 8.72 (1H, s).

mTOR Kinase Assay (Echo): 0.01 μM

### EXAMPLE 4g

¹H NMR (400.132 MHz, DMSO) δ 1.21 (3H, d), 1.50-1.60 (2H, m), 1.75-1.85 (2H, m), 2.18 (6H, s), 2.31 (2H, t), 2.38-2.50 (2H, m), 2.65-2.75 (2H, m), 2.89 (3H, s), 3.15-3.22 (3H, m), 3.50 (1H, dd), 3.65 (1H, dd), 3.75 (1H, dd), 3.96 (1H, dd), 4.22 (1H, dd), 4.55 (1H, s), 6.15 (1H, t), 6.79 (1H, s), 7.48 (2H, d), 8.22 (2H, d), 8.87 (1H, s).

mTOR Kinase Assay (Echo): 0.0626 μM

### EXAMPLE 4h

¹H NMR (400.132 MHz, DMSO) δ 0.88 (3H, t), 1.21 (3H, d), 1.45 (2H, q), 1.50-1.62 (2H, m), 1.75-1.85 (2H, m), 2.38-2.50 (2H, m), 2.65-2.80 (2H, m), 2.89 (3H, s), 3.05 (2H, m), 3.20 (1H, dd), 3.50 (1H, dd), 3.65 (1H, dd), 3.75 (1H, d), 3.96 (1H, dd), 4.22 (1H, d), 4.55 (1H, s), 6.20 (1H, t), 6.78 (1H, s), 7.48 (2H, d), 8.21 (2H, d), 8.62 (1H, s).

mTOR Kinase Assay (Echo): 0.00157 μM

### EXAMPLE 4i

¹H NMR (400.132 MHz, DMSO) δ 0.88 (6H, d), 1.21 (3H, d), 1.50-1.62 (2H, m), 1.70 (1H, m), 1.75-1.85 (2H, m), 2.35-2.50 (2H, m), 2.65-2.80 (2H, m), 2.90 (3H, s), 2.94 (2H, t), 3.20 (1H, dd), 3.50 (1H, dd), 3.65 (1H, dd), 3.75 (1H, dd), 3.97 (1H, dd), 4.24 (1H, d), 4.54 (1H, s), 6.22 (1H, t), 6.78 (1H, s), 7.50 (2H, d), 8.22 (2H, d), 8.63 (1H, s).

mTOR Kinase Assay (Echo): 0.0115 μM

### EXAMPLE 4j

¹H NMR (400.132 MHz, DMSO) δ 1.21 (3H, d), 1.50-1.62 (4H, m), 1.75-1.85 (2H, m), 2.38-2.50 (2H, m), 2.65-2.80 (2H, m), 2.89 (3H, s), 3.10-3.22 (3H, m), 3.45-3.52 (3H, m), 3.65 (1H, dd), 3.75 (1H, d), 3.96 (1H, dd), 4.22 (1H, d), 4.45 (1H, t), 4.55 (1H, s), 6.1991H, t), 6.79 (1H, s), 7.49 (2H, d), 8.22 (2H, d), 8.70 (1H, s).

mTOR Kinase Assay (Echo): 0.00461 μM

### EXAMPLE 4k

¹H NMR (400.132 MHz, DMSO) δ 1.21 (3H, d), 1.50-1.62 (2H, m), 1.75-1.85 (2H, m), 2.38-2.50 (2H, m), 2.65-2.80 (2H, m), 2.89 (3H, s), 3, 22 (1H, dd), 3.50 (1H, dd), 3.65 (1H, dd), 3.75 (1H, d), 3.96 (1H, dd), 4.25 (1H, d), 4.55 (1H, s), 6.80 (1H, s), 7.58 (2H, d), 7.62-7.70 (4H, m), 8.30 (2H, d), 9.04 (1H, s), 9.10 (1H, s).

mTOR Kinase Assay (Echo): 0.00905 μM

### EXAMPLE 4l

¹H NMR (400.132 MHz, DMSO) δ 1.21 (3H, d), 1.55-1.62 (2H, m), 1.78-1.90 (2H, m), 2.38-2.50 (2H, m), 2.65-2.80 (2H, m), 2.89 (3H, s), 3.20 (1H, dd), 3.50 (1H, dd), 3.65 (1H, dd), 3.75 (1H, d), 3.9791H, dd), 4.2591H, d), 4.55 (1H, s), 6.80 (1H, s), 7.02 (1H, dd), 7.58 (1H, d), 7.61 (2H, d), 7.74 (1H, dd), 8.25-8.35 (4H, m), 9.41 (1H, s).

mTOR Kinase Assay (Echo): 0.00369 μM

### EXAMPLE 4m

¹H NMR (400.132 MHz, DMSO) δ 1.20 (3H, d), 1.50-1.62 (2H, m), 1.78-1.85 (2H, m), 2.38-2.50 (2H, m), 2.65-2.80 (2H, m), 2.89 (3H, s), 3.20 (1H, dd), 3.50 (1H, dd), 3.65 (1H, dd), 3.75-40 (4H, m), 3.97 (1H, dd), 4.25 (1H, d), 4.55 (1H, s), 6.80 (1H, s), 7.38 (1H, s), 7.55 (2H, d), 7.73 (1H, s), 8.25 (2H, d), 8.35 (1H, s), 8.85 (1H, s).

mTOR Kinase Assay (Echo): 0.00245 μM

### EXAMPLE 4n

¹H NMR (400.132 MHz, DMSO) δ 1.21 (3H, d), 1.50-1.62 (2H, m), 1.75-1.85 (2H, m), 2.35-2.50 (2H, m), 2.65-2.80 (2H, m), 2.89 (3H, s), 3.20 (1H, dd), 3.50 (1H, dd), 3, 65 (1H, dd), 3.75 (1H, d), 3.97 (1H, dd), 4.24 (1H, d), 4.35 (1H, d), 4.55 (1H, s), 6.65 (1H, t), 6.78 (1H, s), 7.04 (2H, s), 7.52 (2H, d), 8.25 (2H, d), 9.0 (1H, s), 12.6 (1H, s).

mTOR Kinase Assay (Echo): 0.0392 μM

The preparation of phenyl N-[4-[4-[(3S)-3-methylmorpholin-4-yl]-6-(1-methylsulfonylcyclopentyl)pyrimidin-2-yl]phenyl]carbamate is described below.

### Phenyl N-[4-[4-[(3S)-3-methylmorpholin-4-yl]-6-(1-methylsulfonylcyclopentyl)pyrimidin-2-yl]phenyl]carbamate

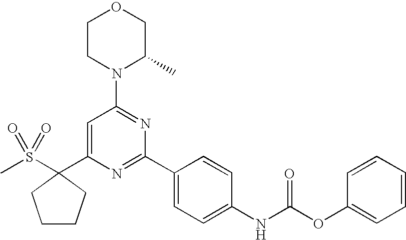

Sodium hydrogen carbonate (1.150 g, 13.68 mmol) was added to 4-[4-[(3S)-3-methylmorpholin-4-yl]-6-(1-methylsulfonylcyclopentyl)pyrimidin-2-yl]aniline (3.8 g, 9.12 mmol), in dioxane (100 mL) at 21° C. under nitrogen. The resulting mixture was cooled to 10° C. and phenyl chloroformate (1.72 mL, 13.68 mmol) added slowly then the reaction stirred for 3 hours and allowed to warm to RT and left for 16 hours. The reaction mixture was diluted with ethyl acetate (250 mL), and washed with water (150 mL). The organic layer was dried (Na₂SO₄), filtered and evaporated to afford crude product. The crude solid was triturated with a mixture of diethyl ether, iso-hexane and acetone to give the desired material as a white solid (4.50 g).

NMR Spectrum: ¹H NMR (400.132 MHz, DMSO-d₆) δ 1.21 (3H, d), 1.50-1.62 (2h, m), 1.75-1.85 (2H, m), 2.38-2.50 (2H, m), 2.65-2.80 (2H, m), 3.21 (1H, dd), 3.30 (3H, s), 3.50 (1H, dd), 3.63 (1H, dd), 3.75 (1H, d), 3.97 (1H, dd), 4.28 (1H, d), 4.57 (1H, s), 6.81 (1H, s), 7.22-7.30 (3Hh, m), 7.43 (2H, dd), 7.61 (1H, d), 8.32 (2H, d), 10.45 (1H, s).

LCMS Spectrum: m/z (ESI+)(M+H)+=537; HPLC tR=2.98 min

### 4-[4-[(3S)-3-Methylmorpholin-4-yl]-6-(1-methylsulfonylcyclopentyl)pyrimidin-2-yl]aniline

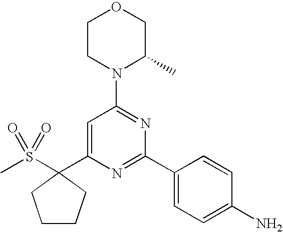

Bis(triphenylphosphine)palladium (II) chloride (0.390 g, 0.56 mmol) was added to 2-chloro-4-[(3S)-3-methylmorpholin-4-yl]-6-(1-methylsulfonylcyclopentyl)pyrimidine (4.0 g, 11.12 mmol), 4-(4,4,5,5-tetramethyl-1,3,2-dioxaborolan-2-yl)aniline (3.17 g, 14.45 mmol) and sodium carbonate (20 mL, 40.0 mmol) in a mixture of DMF (20 mL), DME (50 mL), ethanol (20 mL) and water (20 mL) at RT under nitrogen. The resulting mixture was stirred at 95° C. for 12 hours. The reaction mixture was diluted with ethyl acetate (400 mL), washed twice with water (200 mL followed by 250 mL), the organic layer dried (Na₂SO₄), filtered and evaporated to afford crude product. The crude product was chromatographed on silica, eluting with 5-50% ethyl acetate in iso-hexane, to give a yellow solid which was subsequently triturated with a mixture of diethyl ether and iso-hexane to give the desired material as a white solid (4.25 g).

NMR Spectrum: ¹H NMR (400.132 MHz, DMSO-d₆) δ 1.21 (3H, d), 1.50-1.60 (2H, m), 1.75-1.90 (2H, m), 2.34-2.43 (2H, m), 2.62-2.78 (2H, m), 2.88 (3H, s), 3.18 (1H, dd), 3.48 (1H, dd), 3.65 (1H, dd), 3.75 (1H, dd), 3.95 (1H, dd), 4.20 (1H, d), 4.51 (1H, s), 5.55 (2H, s), 6.62 (2H, d), 6.68 (1H, s), 8.09 (1H, d).

Mass Spectrum: m/z (ESI+)(M+H)+=417

### 2-Chloro-4-[(3S)-3-methylmorpholin-4-yl]-6-(1-methylsulfonylcyclopentyl)pyrimidine

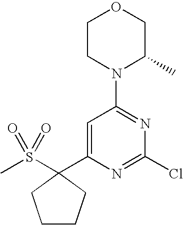

Tetrabutylammonium bromide (0.495 g, 1.54 mmol) was added to a mixture of 2-chloro-4-[(3S)-3-methylmorpholin-4-yl]-6-(methylsulfonylmethyl)pyrimidine (4.7 g, 15.37 mmol), 1,4-dibromobutane (1.84 mL, 15.37 mmol) and aqueous sodium hydroxide solution (30 mL, 368.9 mmol) in DCM (150 mL) at RT under nitrogen. The resulting mixture was stirred at 40° C. for 6 hours. The reaction mixture was diluted with DCM (200 mL), and washed with water (100 mL). The organic layer was dried (MgSO₄), filtered and evaporated to afford crude product. The crude product was chromatographed on silica, eluting with 5-50% ethyl acetate in iso-hexane, to give the desired material as a yellow solid (3.90 g).

NMR Spectrum: ¹H NMR (400.132 MHz, DMSO-d₆) δ 1.20 (3H, d), 1.50-1.60 (2H, m), 1.72-1.82 (2H, m), 2.30-2.41 (2H, m, ), 2.50-2.60 (2H, m), 2.88 (3H, s), 3.20 (1H, dd), 3.45 (1H, dd), 3.60 (1H, dd), 3.71 (1H, d), 3.94 (1H, dd), 4.0-4.10 (1H, m), 4.42 (1H, s), 6.89 (1H, s).

LCMS Spectrum: m/z (ESI+)(M+H)+=360; HPLC tR=2.22 min

The preparation of 2-chloro-4-[(3S)-3-methylmorpholin-4-yl]-6-(methylsulfonylmethyl)pyrimidine was described earlier.

### EXAMPLE 5

### 3-Methyl-1-[4-[4-[(3S)-3-methylmorpholin-4-yl]-6-(1-methylsulfonylcyclobutyl)pyrimidin-2-yl]phenyl]urea

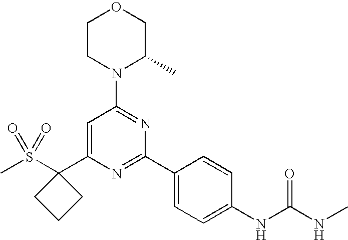

Triethylamine (0.07 mL, 0.48 mmol) was added to a solution of phenyl N-[4-[4-[(3S)-3-methylmorpholin-4-yl]-6-(1-methylsulfonylcyclobutyl)pyrimidin-2-yl]phenyl]carbamate (86 mg, 0.16 mmol) and methylamine (2M in THF, 0.65 mmol) in NMP (2 mL). The reaction was heated at 80° C. for 2 hours the purified by prep HPLC, using a mixture of water (containing 1% NH3) and acetonitrile as eluents, to give the desired material as a solid (48 mg).

NMR Spectrum: ¹H NMR (400.13 MHz, DMSO-d₆) δ 1.24 (3H, d), 1.91 (2H, m), 2.08 (2H, m), 2.62(3H,d), 2.80 (2H, m), 2.87 (3H, s), 3.21 (1H, td), 3.50 (1H, td), 3.65 (1H, dd), 3.77 (1H, d), 3.98 (1H, dd), 4.24 (1H, d), 4.60 (1H, s), 6.07 (1H, d), 6.71 (1H, s), 7.50 (2H, d), 8.22 (2H, d), 8.75 (1H, s).

LCMS Spectrum: m/z (ESI+)(M+H)+=460; HPLC tR=1.5 min

mTOR Kinase Assay (Echo): 0.000802 μM

The following compounds were prepared in an analogous fashion from phenyl N-[4-[4-[(3S)-3-methylmorpholin-4-yl]-6-(1-methylsulfonylcyclobutyl)pyrimidin-2-yl]phenyl]carbamate and the appropriate amine.

| | | | LCMS | Retention |
|---|---|---|---|---|
| Example | Structure | NAME | MH+ | time (min) |
| 5a | 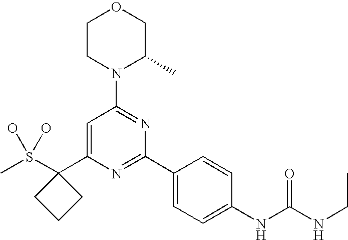 | 3-ethyl-1-[4-[4-[(3S)-3-methylmorpholin-4-yl]-6-(1-methylsulfonylcyclobutyl)pyrimidin-2-yl]phenyl]urea | 474 | 1.98 |
| 5b | 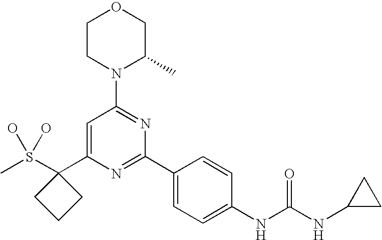 | 3-cyclopropyl-1-[4-[4-[(3S)-3-methylmorpholin-4-yl]-6-(1-methylsulfonylcyclobutyl)pyrimidin-2-yl]phenyl]urea | 486 | 2.01 |
| 5c | 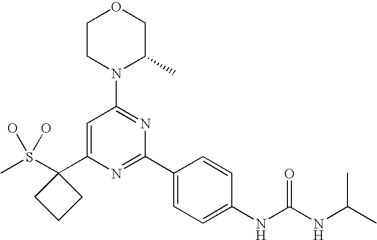 | 1-[4-[4-[(3S)-3-methylmorpholin-4-yl]-6-(1-methylsulfonylcyclobutyl)pyrimidin-2-yl]phenyl]-3-propan-2-yl-urea | 488 | 2.15 |
| 5d | 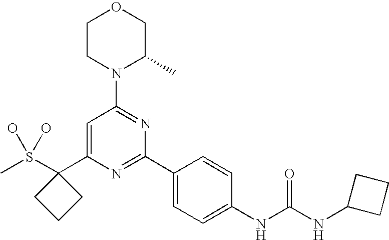 | 3-cyclobutyl-1-[4-[4-[(3S)-3-methylmorpholin-4-yl]-6-(1-methylsulfonylcyclobutyl)pyrimidin-2-yl]phenyl]urea | 500 | 2.24 |
| 5e | 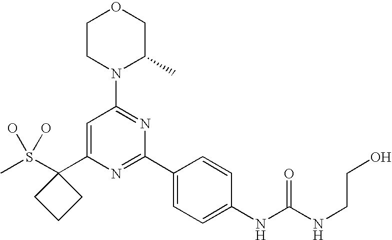 | 3-(2-hydroxymethyl)-1-[4-[4-[(3S)-3-methylmorpholin-4-yl]-6-(1-methylsulfonylcyclobutyl)pyrimidin-2-yl]phenyl]urea | 490 | 1.69 |
| 5f | 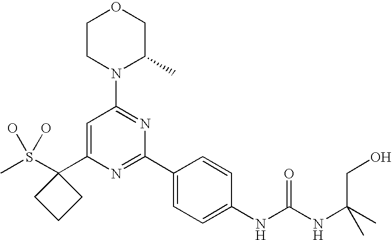 | 3-(1-hydroxy-2-methyl-propan-2-yl)-1-[4-[4-[(3S)-3-methylmorpholin-4-yl]-6-(1-methylsulfonylcyclobutyl)pyrimidin-2-yl]phenyl]urea | 518 | 1.97 |
| 5g | 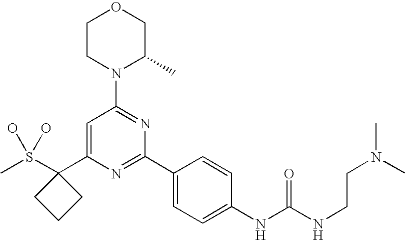 | 3-(2-dimethylaminomethyl)-1-[4-[4-[(3S)-3-methylmorpholin-4-yl]-6-(1-methylsulfonylcyclobutyl)pyrimidin-2-yl]phenyl]urea | 517 | 1.98 |
| 5h | 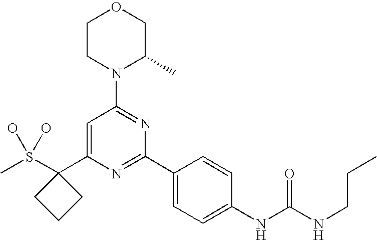 | 1-[4-[4-[(3S)-3-methylmorpholin-4-yl]-6-(1-methylsulfonylcyclobutyl)pyrimidin-2-yl]phenyl]-3-propyl-urea | 488 | 2.16 |
| 5i | 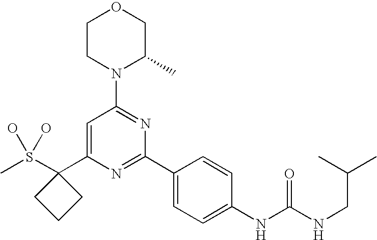 | 1-[4-[4-[(3S)-3-methylmorpholin-4-yl]-6-(1-methylsulfonylcyclobutyl)pyrimidin-2-yl]phenyl]-3-(2-methylpropyl)urea | 502 | 2.33 |
| 5j | 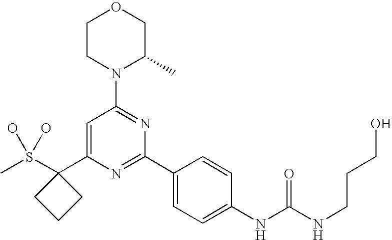 | 3-(3-hydroxypropyl)-1-[4-[4-[(3S)-3-methylmorpholin-4-yl]-6-(1-methylsulfonylcyclobutyl)pyrimidin-2-yl]phenyl]urea | 504 | 1.73 |
| 5k | 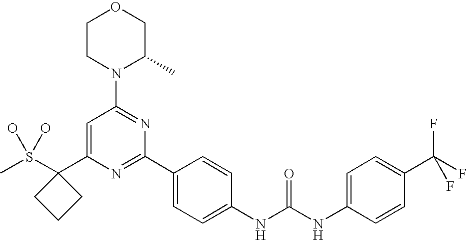 | 1-[4-[4-[(3S)-3-methylmorpholin-4-yl]-6-(1-methylsulfonylcyclobutyl)pyrimidin-2-yl]phenyl]-3-[4-(trifluoromethyl)phenyl]urea | 590 | 2.84 |
| 5l | 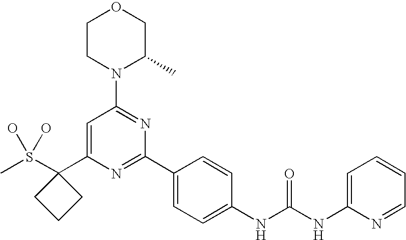 | 1-[4-[4-[(3S)-3-methylmorpholin-4-yl]-6-(1-methylsulfonylcyclobutyl)pyrimidin-2-yl]phenyl]-3-pyridin-2-yl-urea | 523 | 2.42 |
| 5m | 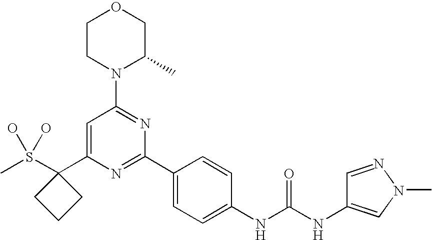 | 1-[4-[4-[(3S)-3-methylmorpholin-4-yl]-6-(1-methylsulfonylcyclobutyl)pyrimidin-2-yl]phenyl]-3-(1-methylpyrazol-4-yl)urea | 526 | 1.88 |
| 5n | 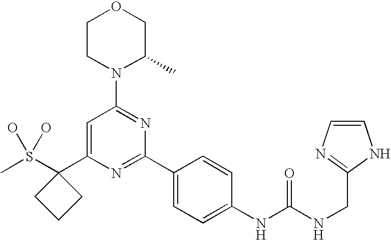 | 3-(1H-imidazol-2-ylmethyl)-1-[4-[4-[(3S)-3-methylmorpholin-4-yl]-6-(1-methylsulfonylcyclobutyl)pyrimidin-2-yl]phenyl]urea | 526 | 1.79 |

### EXAMPLE 5a

¹H NMR (400.132 MHz, DMSO) δ 1.07 (3H, t), 1.24 (3H, d), 1.91 (2H, m), 2.07 (2H, m), 2.81 (2H, m), 2.87 (3H, s), 3.12 (2H, m), 3.22 (1H, m), 3.50 (1H, td), 3.65 (1H, dd), 3.77 (1H, d), 3.98 (1H, dd), 4.24 (1H, d), 4.59 (1H, s), 6.16 (1H, t), 6.71 (1H, s), 7.49 (2H, d), 8.22 (2H, d), 8.68 (1H, s).

mTOR Kinase Assay (Echo): 0.000289 μM

### EXAMPLE 5b

¹H NMR (400.132 MHz, DMSO) δ 0.42 (2H, m), 0.65 (2H, m), 1.24 (3H, d), 1.92 (2H, m), 2.08 (2H, m), 2.56 (2H, m), 2.80 (2H, m), 2.88 (3H, s), 3.21 (1H, td), 3.54 (1H, s), 3.65 (1H, dd), 3.77 (1H, d), 3.98 (1H, dd), 4.24 (1H, d), 4.59 (1H, s), 6.45 (1H, s), 6.72 (1H, s), 7.51 (2H, d), 8.22 (2H, d), 8.55 (1H, s).

mTOR Kinase Assay (Echo): 0.000383 μM

### EXAMPLE 5c

¹H NMR (400.132 MHz, DMSO) δ 1.11 (6H, d), 1.24 (3H, d), 1.86-1.96 (2H, m), 2.03-2.13 (2H, m), 2.76-2.84 (2H, m), 2.86 (3H, s), 3.21 (1H, td), 3.50 (1H, td), 3.65 (1H, dd), 3.73-3.80 (2H, m), 3.98 (1H, dd), 4.25 (1H, d), 4.61 (1H, s), 6.05 (1H, d), 6.72 (1H, s), 7.48 (2H, d), 8.21 (2H, d), 8.54 (1H, s).

mTOR Kinase Assay (Echo): 0.00681 μM

### EXAMPLE 5d

¹H NMR (400.132 MHz, DMSO) δ 1.23 (3H, d), 1.58-1.67 (3H, m), 1.81-1.94 (4H, m), 2.02-2.11 (2H, m), 2.20 (2H, m), 2.76-2.84 (2H, m), 2.87 (3H, s), 3.21 (1H, td), 3.50 (1H, td), 3.65 (1H, dd), 3.77 (1H, d), 3.98 (1H, dd), 4.13 (1H, quintet), 4.25 (1H, d), 4.61 (1H, s), 6.45 (1H, d), 6.71 (1H, s), 7.47 (2H, d), 8.21 (3H, d), 8.58 (2H, s).

mTOR Kinase Assay (Echo): 0.00385 μM

### EXAMPLE 5e

¹H NMR (400.132 MHz, DMSO) δ 1.24 (3H, d), 1.87-1.97 (2H, m), 2.01-2.12 (2H, m), 2.77-2.85 (2H, m), 2.88 (3H, s), 3.15-3.25 (3H, m), 3.43-3.54 (3H, m), 3.65 (1H, dd), 3.77 (1H, d), 3.98 (1H, dd), 4.24 (1H, d), 4.60 (1H, s), 4.72 (1H, t), 6.26 (1H, t), 6.71 (1H, s), 7.49 (2H, d), 8.23 (2H, d), 8.82 (1H, s).

mTOR Kinase Assay (Echo): 0.000864 μM

### EXAMPLE 5f

¹H NMR (400.132 MHz, DMSO) δ 1.25 (14H, s), 1.87-1.95 (2H, m), 2.03-2.11 (2H, m), 2.76-2.83 (7H, m), 2.88 (7H, s), 3.17-3.25 (15H, m), 3.39 (2H, d), 3.65 (1H, dd), 3.77 (1H, d), 3.98 (1H, d), 4.24 (1H, d), 4.59 (1H, s), 4.95 (1H, t), 6.02 (1H, s), 6.72 (1H, s), 7.44 (2H, d), 8.21 (2H, d), 8.75 (1H, s).

mTOR Kinase Assay (Echo): 0.00736 μM

### EXAMPLE 5g

¹H NMR (400.132 MHz, DMSO) δ 1.24 (3H, d), 1.87-1.96 (2H, m), 2.02-2.12 (2H, m), 2.21 (6H, s), 2.34 (2H, t), 2.74-2.85 (2H, m), 2.88 (3H, s), 3.15-3.26 (3H, m), 3.50 (1H, td), 3.65 (1H, dd), 3.77 (1H, d), 3.98 (1H, dd), 4.24 (1H, d), 4.60 (1H, s), 6.15 (1H, t), 6.72 (1H, s), 7.48 (2H, d), 8.21 (2H, d), 8.91 (1H, s).

mTOR Kinase Assay (Echo): 0.0668 μM

### EXAMPLE 5h

¹H NMR (400.132 MHz, DMSO) δ 0.89 (3H, t), 1.24 (3H, d), 1.40-1.51 (2H, m), 1.87-1.97 (2H, m), 2.01-2.12 (2H, m), 2.76-2.84 (2H, m), 2.89 (3H, s), 3.06 (2H, q), 3.17-3.27 (1H, m), 3.50 (1H, td), 3.65 (1H, dd), 3.77 (1H, d), 3.98 (1H, dd), 4.24 (1H, d), 4.57 (1H, s), 6.20 (1H, t), 6.72 (1H, s), 7.49 (2H, d), 8.21 (2H, d), 8.66 (1H, s).

mTOR Kinase Assay (Echo): 0.00234 μM

### EXAMPLE 5i

¹H NMR (400.132 MHz, DMSO) δ 0.89 (6H, d), 1.24 (3H, d), 1.65-1.75 (2H, m), 1.85-1.96 (2H, m), 2.01-2.12 (2H, m), 2.75-2.84 (2H, m), 2.88 (3H, s), 2.89-2.99 (3H, m), 3.16-3.26 (1H, m), 3.50 (1H, td), 3.65 (1H, dd), 3.77 (1H, d), 3.98 (1H, dd), 4.24 (1H, d), 4.56 (1H, s), 6.24 (1H, t), 6.72 (1H, s), 7.49 (2H, d), 8.22 (2H, d), 8.65 (1H, s).

mTOR Kinase Assay (Echo): 0.00988 μM

### EXAMPLE 5j

¹H NMR (400.132 MHz, DMSO) δ 1.24 (3H, d), 1.59 (2H, q), 1.86-1.96 (2H, m), 2.02-2.14 (2H, m), 2.76-2.85 (2H, m), 2.88 (3H, s), 3.13-3.26 (3H, m), 3.45-3.54 (3H, m), 3.65 (1H, dd), 3.77 (1H, d), 3.98 (1H, dd), 4.24 (1H, d), 4.48 (1H, t), 4.56 (1H, s), 6.20 (1H, t), 6.72 (1H, s), 7.49 (2H, d), 8.21 (2H, d), 8.73 (1H, s).

mTOR Kinase Assay (Echo): 0.000239 μM

### EXAMPLE 5k

mTOR Kinase Assay (Echo): 0.00333 μM

### EXAMPLE 5l

mTOR Kinase Assay (Echo): 0.000248 μM

### EXAMPLE 5m

¹H NMR (400.132 MHz, DMSO) δ 1.24 (3H, d), 1.87-1.98 (2H, m), 2.03-2.12 (2H, m), 2.76-2.84 (2H, m), 2.88 (3H, s), 3.17-3.30 (4H, m), 3.51 (1H, td), 3.65 (1H, dd), 3.73-3.80 (1H, m), 3.98 (1H, dd), 4.25 (1H, d), 4.62 (1H, s), 6.73 (1H, s), 7.39 (1H, s), 7.54 (2H, d), 7.77 (1H, s), 8.27 (2H, d), 8.40 (1H, s), 8.86 (1H, s).

mTOR Kinase Assay (Echo): 0.00069 μM

### EXAMPLE 5n

¹H NMR (400.132 MHz, DMSO) δ 1.24 (3H, d), 1.87-1.97 (2H, m), 2.03-2.12 (2H, m), 2.75-2.85 (2H, m), 2.88 (3H, s), 3.21 (1H, td), 3.50 (1H, td), 3.65 (1H, dd), 3.77 (1H, d), 3.98 (1H, dd), 4.25 (1H, d), 4.32 (2H, d), 4.60 (1H, s), 6.62 (1H, t), 6.98 (2H, s), 7.52 (2H, d), 8.24 (2H, d), 11.88 (1H, s).

mTOR Kinase Assay (Echo): 0.00828 μM

The preparation of phenyl N-[4-[4-[(3S)-3-methylmorpholin-4-yl]-6-(1-methylsulfonylcyclobutyl)pyrimidin-2-yl]phenyl]carbamate is described below.

### Phenyl N-[4-[4-[(3S)-3-methylmorpholin-4-yl]-6-(1-methylsulfonylcyclobutyl)pyrimidin-2-yl]phenyl]carbamate

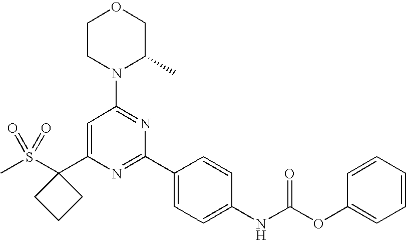

Sodium hydrogen carbonate (0.313 g, 3.73 mmol) was added to 4-[4-[(3S)-3-methylmorpholin-4-yl]-6-(1-methylsulfonylcyclobutyl)pyrimidin-2-yl]aniline (1 g, 2.48 mmol), in dioxane (20 mL) at RT under nitrogen. The resulting mixture was cooled to 10° C. and phenyl chloroformate (0.468 mL, 3.73 mmol) added slowly. The reaction was stirred for 3 hours then diluted with ethyl acetate (150 mL), and washed with water (100 mL). The organic layer was dried (Na₂SO₄), filtered and evaporated. The crude solid was triturated with a mixture of diethyl ether, iso-hexane and acetone to give the desired material as a white solid (1.35 g).

NMR Spectrum: ¹H NMR (400.13 MHz, DMSO-d₆) δ 1.21(3H,d), 1.88-1.96(2H,m), 2.02-2.11(2H,m), 2.75-2.85(2H,m), 2.85(3H,s), 3.21(1H,dd), 3.50(1H,dd), 3.64(1H,d), 3.75(1H,d), 3.98(1H,dd), 4.25(1H,d), 4.57(1H,s), 6.72(1H,s), 7.20-7.30(3H,m), 7.42(2H,dd), 7.61(2H,d), 8.32(2H,m), 10.44(1H,s).

LCMS Spectrum: m/z (ESI+)(M+H)+=523; HPLC tR=2.88 min

### 4-[4-[(3S)-3-Methylmorpholin-4-yl]-6-(1-methylsulfonylcyclobutyl)pyrimidin-2-yl]aniline

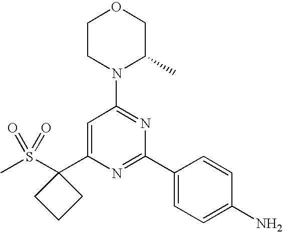

Bis(triphenylphosphine)palladium(II) chloride (0.101 g, 0.14 mmol) was added to 2-chloro-4-[(3S)-3-methylmorpholin-4-yl]-6-(1-methylsulfonylcyclobutyl)pyrimidine (1 g, 2.89 mmol), 4-(4,4,5,5-tetramethyl-1,3,2-dioxaborolan-2-yl)aniline (0.824 g, 3.76 mmol) and sodium carbonate (5 mL, 10.00 mmol) in a mixture of DMF (5 mL), DME (50 mL), ethanol (20 mL) and water (20 mL) at RT under nitrogen. The resulting mixture was stirred at 95° C. for 12 hours. The reaction mixture was diluted with ethyl acetate (400 mL), and washed twice with water (200 mL followed by 250 mL), the organic layer dried (Na₂SO₄), filtered and evaporated. The crude product was chromatographed on silica, eluting with 5-60% ethyl acetate in iso-hexane, to give the desired material as a cream solid (0.98 g).

NMR Spectrum: ¹H NMR (400.13 MHz, DMSO-d₆) δ 1.21(3H,d), 1.85-1.95(2H,m), 2.0-2.10(2H,m), 2.71-2.82(2H,m), 2.82(3H,s), 3.18(1H,dd), 3.50(1H,dd), 3.62(1H,dd), 3.75(1H,d), 3.95(1H,dd), 4.20(1H,d), 4.53(1H,s), 5.55(2H,s), 6.60(3H,d), 8.05(2H,d).

LCMS Spectrum: m/z (ESI+)(M+H)+=403; HPLC tR=2.17 min

### 2-Chloro-4-[(3S)-3-methylmorpholin-4-yl]-6-(1-methylsulfonylcyclobutyl)pyrimidine

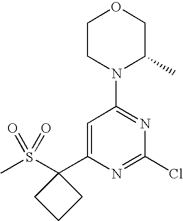

Tetrabutylammonium bromide (0.45 g, 1.40 mmol) was added to 2-chloro-4-[(3S)-3-methylmorpholin-4-yl]-6-(methylsulfonylmethyl)pyrimidine (4.27 g, 13.96 mmol), 1,3-dibromopropane (1.42 mL, 13.96 mmol) and aqueous sodium hydroxide solution (30 mL, 368.9 mmol) in DCM (100 mL) at RT under nitrogen. The resulting mixture was stirred at 35° C. for 5 hours then diluted with DCM (50 mL), and washed with water (25 mL). The organic layer was dried (MgSO₄), filtered and evaporated. The crude product was chromatographed on silica, eluting with 5-50% ethyl acetate in iso-hexane, to give the desired material (1.0 g).

LCMS Spectrum: m/z (ESI+)(M+H)+=346; HPLC tR=1.92 min

The preparation of 2-chloro-4-[(3S)-3-methylmorpholin-4-yl]-6-(methylsulfonylmethyl)pyrimidine was described earlier.

### EXAMPLE 6

### 3-Cyclobutyl-1-[4-[4-[(3S)-3-methylmorpholin-4-yl]-6-(1-pyridin-4-ylsulfonylcyclopropyl)pyrimidin-2-yl]phenyl]urea

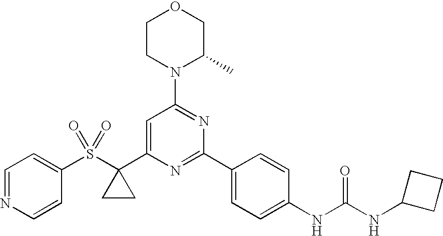

To a solution of phenyl N-[4-[4-[(3S)-3-methylmorpholin-4-yl]-6-(1-pyridin-4-ylsulfonylcyclopropyl)pyrimidin-2-yl]phenyl]carbamate (122 mg, 0.21 mmol) in DMF (2 mL) was added triethylamine (0.088 mL, 0.63 mmol) followed by cyclobutylamine (0.090 mL, 1.05 mmol) and the reaction heated at 50° C. overnight. The crude product was purified by preparative HPLC using decreasingly polar mixtures of water (containing 1% NH3) and acetonitrile as eluents, to give the desired material as a white solid (90 mg).

NMR Spectrum: ¹H NMR (400.132 MHz, DMSO-d₆) δ 1.18-1.20 (3H, d), 1.57-1.70 (4H, m), 1.82-1.91 (2H, m), 1.95-1.98 (2H, q), 2.18-2.25 (2H, m), 3.12-3.20 (1H, td), 3.43-3.50 (1H, td), 3.59-3.63 (1H, dd), 3.73-3.76 (1H, d), 3.94-3.98 (1H, dd), 4.11-4.17 (2H, m), 4.46 (1H, bs), 6.42-6.44 (1H, d), 6.67 (1H, s), 7.33-7.35 (2H, q), 7.65-7.67 (2H, d), 7.76-7.78 (2H, q), 8.53 (1H, s), 8.85-8.87 (2H, q).

LCMS Spectrum: m/z (ESI+) (M+H)+549=HPLC tR=2.25 min.

mTOR Kinase Assay (Echo): 0.001 μM

The compounds below were prepared in an analogous fashion from phenyl N-[4-[4-[(3S)-3-methylmorpholin-4-yl]-6-(1-pyridin-4-ylsulfonylcyclopropyl)pyrimidin-2-yl]phenyl]carbamate using the appropriate amine.

| | | | LCMS | Retention |
|---|---|---|---|---|
| Example | Structure | NAME | MH+ | time (min) |
| 6a | 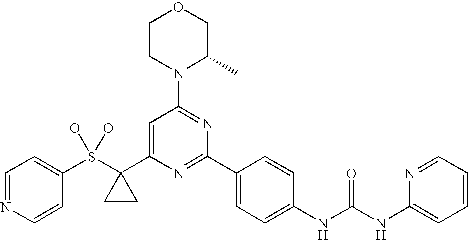 | 1-[4-[4-[(3S)-3-methylmorpholin-4-yl]-6-(1-pyridin-4-ylsulfonylcyclopropyl)pyrimidin-2-yl]phenyl]-3-pyridin-2-yl-urea | 572 | 2.44 |
| 6b | 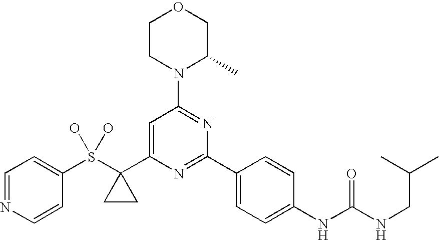 | 1-[4-[4-[(3S)-3-methylmorpholin-4-yl]-6-(1-pyridin-4-ylsulfonylcyclopropyl)pyrimidin-2-yl]phenyl]-3-(2-methylpropyl)urea | 551 | 2.33 |
| 6c | 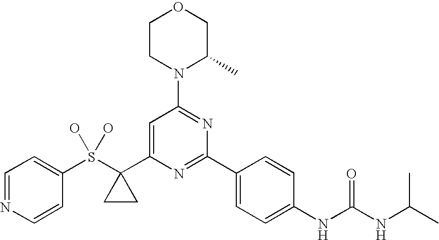 | 1-[4-[4-[(3S)-3-methylmorpholin-4-yl]-6-(1-pyridin-4-ylsulfonylcyclopropyl)pyrimidin-2-yl]phenyl]-3-propan-2-yl-urea | 537 | 2.17 |
| 6d | 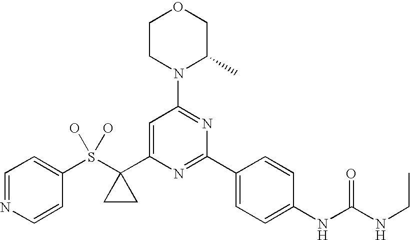 | 3-ethyl-1-[4-[4-[(3S)-3-methylmorpholin-4-yl]-6-(1-pyridin-4-ylsulfonylcyclopropyl)pyrimidin-2-yl]phenyl]urea | 523 | 2.03 |
| 6e | 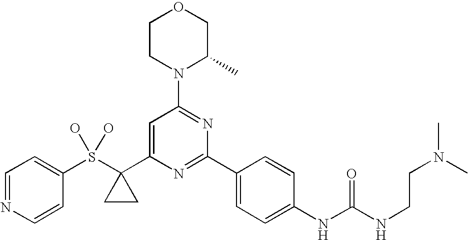 | 3-(2-dimethylaminoethyl)-1-[4-[4-[(3S)-3-methylmorpholin-4-yl]-6-(1-pyridin-4-ylsulfonylcyclopropyl)pyrimidin-2-yl]phenyl]urea | 566 | 1.32 |
| 6f | 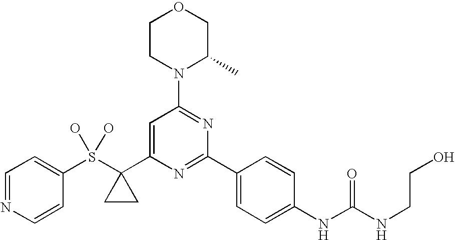 | 3-(2-hydroxyethyl)-1-[4-[4-[(3S)-3-methylmorpholin-4-yl]-6-(1-pyridin-4-ylsulfonylcyclopropyl)pyrimidin-2-yl]phenyl]urea | 539 | 1.75 |
| 6g | 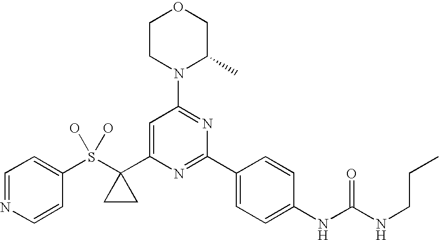 | 1-[4-[4-[(3S)-3-methylmorpholin-4-yl]-6-(1-pyridin-4-ylsulfonylcyclopropyl)pyrimidin-2-yl]phenyl]-3-propyl-urea | 537 | 2.18 |
| 6h | 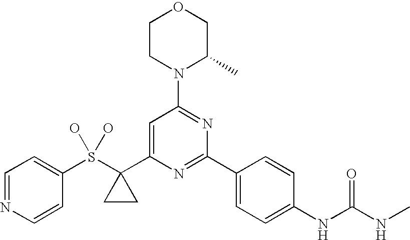 | 3-methyl-1-[4-[4-[(3S)-3-methylmorpholin-4-yl]-6-(1-pyridin-4-ylsulfonylcyclopropyl)pyrimidin-2-yl]phenyl]urea | 509 | 1.89 |
| 6i | 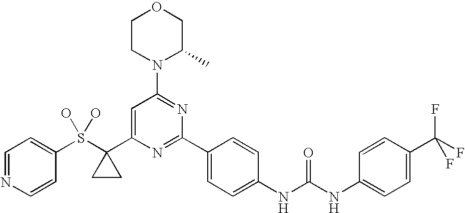 | 1-[4-[4-[(3S)-3-methylmorpholin-4-yl]-6-(1-pyridin-4-ylsulfonylcyclopropyl)pyrimidin-2-yl]phenyl]-3-[4-(trifluoromethyl)phenyl]urea | 639 | 2.84 |
| 6j | 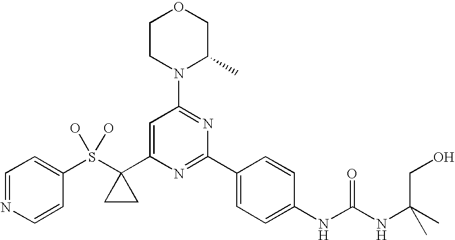 | 3-(1-hydroxy-2-methyl-propan-2-yl)-1-[4-[4-[(3S)-3-methylmorpholin-4-yl]-6-(1-pyridin-4-ylsulfonylcyclopropyl)pyrimidin-2-yl]phenyl]urea | 567 | 2.01 |
| 6k | 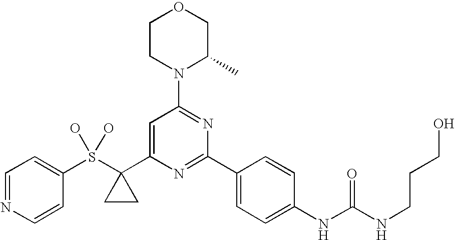 | 3-(2-hydroxypropyl-1-[4-[4-[(3S)-3-methylmorpholin-4-yl]-6-(1-pyridin-4-ylsulfonylcyclopropyl)pyrimidin-2-yl]phenyl]urea | 553 | 1.80 |
| 6l | 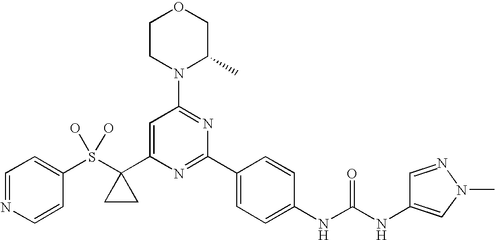 | 1-[4-[4-[(3S)-3-methylmorpholin-4-yl]-6-(1-pyridin-4-ylsulfonylcyclopropyl)pyrimidin-2-yl]phenyl]-3-(1-methylpyrazol-4-yl)urea | 575 | 1.96 |
| 6m | 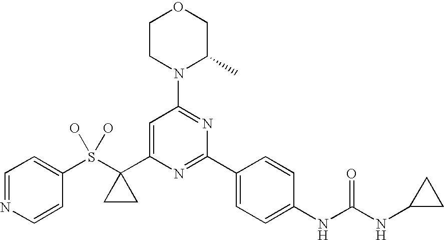 | 3-cyclopropyl-1-[4-[4-[(3S)-3-methylmorpholin-4-yl]-6-(1-pyridin-4-ylsulfonylcyclopropyl)pyrimidin-2-yl]phenyl]urea | 535 | 2.12 |

### EXAMPLE 6a

¹H NMR (400.132 MHz, DMSO-d₆) δ 1.19-1.21 (3H, d), 1.69-1.72 (2H, q), 1.97-2.00 (2H, q), 3.15-3.22 (1H, td), 3.44-3.51 (1H, td), 3.61-3.64 (1H, dd), 3.74-3.77 (1H, d), 3.95-3.99 (1H, dd), 4.17-4.20 (1H, d), 4.48 (1H, bs), 6.71 (1H, s), 7.02-7.06 (1H, m), 7.49-7.51 (2H, d), 7.56-7.58 (1H, d), 7.74-7.79 (5H, m), 8.30-8.32 (1H, d), 8.88-8.89 (2H, q), 9.43 (1H, s), 10.55 (1H, s).

mTOR Kinase Assay (Echo): 0.00293 μM

### EXAMPLE 6b

¹H NMR (400.132 MHz, DMSO-d₆) δ 0.88-0.90 (6H, d), 1.18-1.20 (3H, d), 1.68-1.74 (3H, m), 1.96-1.98 (2H, q), 2.93-2.96 (2H, t), 3.13-3.20 (1H, td), 3.43-3.50 (1H, td), 3.59-3.63 (1H, dd), 3.73-3.76 (1H, d), 3.94-3.99 (1H, dd), 4.15-4.18 (1H, d), 4.45 (1H, bs), 6.20-6.23 (1H, t), 6.67 (1H, s), 7.34-7.36 (2H, d), 7.65-7.67 (2H, d), 7.77-7.78 (2H, q), 8.62 (1H, s), 8.86-8.87 (2H, q).

mTOR Kinase Assay (Echo): 0.00612 μM

### EXAMPLE 6c

¹H NMR (400.132 MHz, DMSO-d₆) δ 1.11-1.12 (6H, d), 1.18-1.20 (3H, d), 1.67-1.71 (2H, q), 1.96-1.98 (2H, q), 3.13-3.20 (1H, td), 3.43-3.50 (1H, td), 3.59-3.63 (1H, dd), 3.73-3.82 (2H, m), 3.94-3.98 (1H, dd), 4.14-4.18 (1H, d), 4.46 (1H, bs), 6.02-6.04 (1H, d), 6.67 (1H, s), 7.32-7.36 (2H, q), 7.64-7.67 (2H, q), 7.76-7.78 (2H, q), 8.50 (1H, s), 8.86-8.87 (2H, q).

mTOR Kinase Assay (Echo): 0.00321 μM

### EXAMPLE 6d

¹H NMR (400.132 MHz, DMSO-d₆) δ 1.05-1.09 (2H, t), 1.18-1.20 (3H, d), 1.67-1.71 (2H, q), 1.96-1.98 (2H, q), 3.09-3.20 (4H, m), 3.43-3.50 (1H, td), 3.59-3.63 (1H, dd), 3.73-3.76 (1H, d), 3.94-3.98 (1H, dd), 4.15-4.17 (1H, d), 4.46 (1H, bs), 6.12-6.15 (1H, t), 6.67 (1H, s), 7.35-7.37 (2H, q), 7.65-7.67 (2H, d), 7.77-7.78 (2H, q), 8.63 (1H, s), 8.86-8.87 (2H, q).

mTOR Kinase Assay (Echo): 0.000874 μM

### EXAMPLE 6e

¹H NMR (400.132 MHz, DMSO-d₆) δ 1.18-1.20 (3H, d), 1.67-1.71 (2H, q), 1.96-1.98 (2H, q), 2.18 (6H, s), 2.32-2.35 (2H, t), 3.13-3.21 (3H, m), 3.43-3.50 (1H, td), 3.59-3.63 (1H, dd), 3.73-3.76 (1H, d), 3.94-3.98 (1H, dd), 4.15-4.18 (1H, d), 4.47 (1H, bs), 6.12-6.15 (1H, t), 6.67 (1H, s), 7.34-7.37 (2H, q), 7.65-7.67 (2H, d), 7.76-7.78 (2H, q), 8.86-8.87 (3H, m).

mTOR Kinase Assay (Echo): 0.0673 μM

### EXAMPLE 6f

¹H NMR (400.132 MHz, DMSO-d₆) δ 1.18-1.20 (3H, d), 1.67-1.71 (2H, q), 1.96-1.98 (2H, q), 3.13-3.20 (3H, m), 3.43-3.50 (3H, m), 3.59-3.63 (1H, dd), 3.73-3.76 (1H, d), 3.94-3.98 (1H, dd), 4.15-4.18 (1H, d), 4.46 (1H, bs), 4.71-4.74 (1H, t), 6.21-6.24 (1H, t), 6.67 (1H, s), 7.34-7.36 (2H, q), 7.65-7.67 (2H, d), 7.76-7.78 (2H, q), 8.77 (1H, s), 8.86-8.87 (2H, q).

mTOR Kinase Assay (Echo): 0.000794 μM

### EXAMPLE 6g

¹H NMR (400.132 MHz, DMSO-d₆) δ 0.87-0.91 (3H, t), 1.18-1.20 (3H, d), 1.41-1.50 (2H, m), 1.67-1.71 (2H, q), 1.96-1.98 (2H, q), 3.04-3.09 (2H, q), 3.13-3.19 (1H, td), 3.43-3.50 (1H, td), 3.59-3.63 (1H, td), 3.73-3.76 (1H, d), 3.94-3.98 (1H, dd), 4.14-4.18 (1H, d), 4.47 (1H, bs), 6.16-6.19 (1H, t), 6.67 (1H, s), 7.34-7.36 (2H, d), 7.65-7.67 (2H, d0, 7.77-7.78 (2H, q), 8.62 (1H, s), 8.86-8.87 (2H, q).

mTOR Kinase Assay (Echo): 0.00225 μM

### EXAMPLE 6h

¹H NMR (400.132 MHz, DMSO-d₆) δ 1.18-1.20 (3H, d), 1.67-1.71 (2H, q), 1.96-1.98 (2H, q), 2.65-2.67 (3H, d), 3.13-3.20 (1H, td), 3.43-3.50 (1H, td), 3.59-3.63 (1H, dd), 3.73-3.76 (1H, d), 3.94-3.98 (1H, dd), 4.15-4.17 (1H, d), 4.46 (1H, bs), 6.02-6.06 (1H, q), 6.67 (1H, s), 7.35-7.38 (2H, q), 7.65-7.67 (2H, d), 7.77-7.78 (2H, q), 8.71 (1H, s), 8.86-8.87 (2H, q).

mTOR Kinase Assay (Echo): 0.000799 μM

### EXAMPLE 6i

¹H NMR (400.132 MHz, DMSO-d₆) δ 1.19-1.21 (3H, d), 1.69-1.72 (2H, q), 1.97-2.00 (2H, q), 3.14-3.21 (1H, td), 3.44-3.50 (1H, td), 3.61-3.64 (1H, dd), 3.74-3.77 (1H, d), 3.95-3.98 (1H, dd), 4.16-1.20 (1H, d), 4.48 (1H, bs), 6.70 (1H, s), 7.44-7.46 (2H, d), 7.64-7.70 (4H, q), 7.72-7.75 (2H, q), 7.78-7.79 (2H, q), 8.87-8.89 (2H, q), 9.03 (1H, s), 9.11 (1H, s).

mTOR Kinase Assay (Echo): 0.00462 μM

### EXAMPLE 6j

¹H NMR (400.132 MHz, DMSO-d₆) δ 1.18-1.20 (3H, d), 1.24 (6H, s), 1.67-1.70 (2H, q), 1.96-1.99 (2H, q), 3.13-3.20 (1H, td), 3.38-3.40 (2H, d), 3.43-3.50 (1H, td), 3.59-3.63 (1H, dd), 3.73-3.76 (1H, d), 3.94-3.98 (1H, dd), 4.15-4.18 (1H, d), 4.46 (1H, bs), 4.94-4.96 (1H, t), 5.97 (1H, s), 6.67 (1H, s), 7.30-7.32 (2H, d), 7.63-7.65 (2H, q), 7.76-7.78 (2H, q), 8.71 (1H, s), 8.86-8.87 (2H, q).

mTOR Kinase Assay (Echo): 0.00593 μM

### EXAMPLE 6k

¹H NMR (400.132 MHz, DMSO-d₆) δ 1.18-1.20 (3H, d), 1.57-1.63 (2H, m), 1.67-1.71 (2H, q), 1.96-1.98 (2H, q), 3.13-3.20 (3H, m), 3.43-3.50 (3H, m), 3.59-3.63 (1H, dd), 3.73-3.76 (1H, d), 3.94-3.98 (1H, dd), 4.14-4.18 (1H, d), 4.46 (1H, bs), 4.46-4.49 (1H, t), 6.16-6.19 (1H, t), 6.67 (1H, s), 7.34-7.36 (2H, q), 7.65-7.67 (2H, d), 7.76-7.78 (2H, q), 8.68 (1H, s), 8.86-8.87 (2H, q).

mTOR Kinase Assay (Echo): 0.00186 μM

### EXAMPLE 6l

¹H NMR (400.132 MHz, DMSO-d₆) δ 1.19-1.20 (3H, d), 1.68-1.71 (2H, q), 1.96-1.99 (2H, q), 3.13-3.21 (1H, td), 3.44-3.50 (1H, td), 3.60-3.63 (1H, dd), 3.74-3.77 (1H, d), 3.79 (3H, s), 3.95-3.98 (1H, dd), 4.16-4.19 (1H, d), 4.47 (1H, bs), 6.68 (1H, s), 7.39-7.42 (3H, m), 7.68-7.70 (2H, d), 7.77-7.79 (3H, m), 8.37 (1H, s), 8.82 (1H, s), 8.86-8.88 (2H, q).

mTOR Kinase Assay (Echo): 0.00119 μM

### EXAMPLE 6m

¹H NMR (400.132 MHz, DMSO-d₆) δ 0.40-0.44(2H, m), 0.63-0.67(2H, m), 1.18-1.20(3H, d), 1.68-1.71(2H, q), 1.96-1.99(2H, q), 2.53-2.59(1H, m), 3.13-3.20(1H, td), 3.43-3.63(1H, td), 3.59-3.63(1H, dd), 3.73-3.76(1H, d), 3.94-3.98(1H, dd), 4.15-4.18(1H, d), 4.46(1H, bs), 6.40-6.41(1H, d), 6.67(1H, s), 7.36-7.38(2H, q), 7.65-7.68(2H, q), 7.77-7.78(2H, q), 8.51(1H, s), 8.86-8.87(2H, q).

mTOR Kinase Assay (Echo): 0.000936 μM

The preparation of phenyl N-[4-[4-[(3S)-3-methylmorpholin-4-yl]-6-(1-pyridin-4-ylsulfonylcyclopropyl)pyrimidin-2-yl]phenyl]carbamate is described below.

### Phenyl N-[4-[4-[(3S)-3-methylmorpholin-4-yl]-6-(1-pyridin-4-ylsulfonylcyclopropyl)pyrimidin-2-yl]phenyl]carbamate

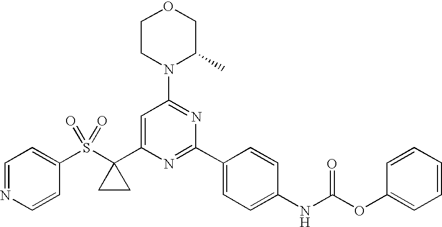

Phenyl chloroformate (0.341 mL, 2.71 mmol) was added to 4-[4-[(3S)-3-methylmorpholin-4-yl]-6-(1-pyridin-4-ylsulfonylcyclopropyl)pyrimidin-2-yl]aniline (1.224 g, 2.71 mmol) and sodium bicarbonate (0.342 g, 4.07 mmol) in dioxane (175 mL) at RT under air. The resulting slurry was stirred at RT for 2 hours. Additional portions of phenyl chloroformate (2×0.005 mL) were added to the reaction. Water was added to the reaction mixture and the material extracted with DCM. The combined organics were dried (MgSO₄), filtered and evaporated. The crude product was purified by flash silica chromatography, eluting with 0 to 4% methanol in DCM, to give the desired material as a beige solid (1.72 g).

NMR Spectrum: ¹H NMR (400.132 MHz, CDCl₃) δ 1.28-1.29 (3H, d), 1.60-1.69 (2H, m), 1.97-2.05 (2H, m), 3.22-3.30 (1H, td), 3.52-3.59 (1H, td), 3.71-3.72 (1H, dd), 3.78-3.81 (1H, d), 3.99-4.03 (1H, dd), 4.09-4.13 (1H, d), 4.38-4.39 (1H, bs), 6.72 (1H, s), 7.13-7.15 (2H, d), 7.19-7.21 (1H, t), 7.32-7.36 (2H, t), 7.46-7.48 (2H, d), 7.61-7.63 (2H, q), 7.97-7.99 (2H, d), 8.74-8.75 (2H, q).

LCMS Spectrum: m/z (ES+) (M+H)+=450; HPLC tR=2.66 min.

### 4-[4-[(3S)-3-Methylmorpholin-4-yl]-6-(1-pyridin-4-ylsulfonylcyclopropyl)pyrimidin-2-yl]aniline

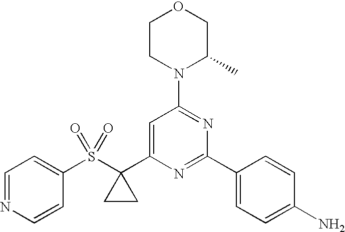

trans-Dichlorobis(triphenylphosphine)palladium (II) (0.095 g, 0.14 mmol) was added to 2-chloro-4-[(3S)-3-methylmorpholin-4-yl]-6-(1-pyridin-4-ylsulfonylcyclopropyl)pyrimidine (1.07 g, 2.71 mmol), 4-(4,4,5,5-tetramethyl-1,3,2-dioxaborolan-2-yl)aniline (0.89 g, 4.06 mmol) and sodium carbonate (6.77 mL, 13.55 mmol) in 18% DMF in a 7:3:2 mixture of DME:water:ethanol (50 mL) at RT under nitrogen. The resulting solution was stirred at 80° C. for 5 hours. The reaction was cooled and diluted with ethyl acetate and water. The reaction mixture was extracted with ethyl acetate, the combined organics dried (MgSO₄), filtered and evaporated to afford the desired material (1.224 g).

LCMS Spectrum: m/z (ES+) (M+H)+=452; HPLC tR=2.03 min.

### 2-Chloro-4-[(3S)-3-methylmorpholin-4-yl]-6-(1-pyridin-4-ylsulfonylcyclopropyl)pyrimidine

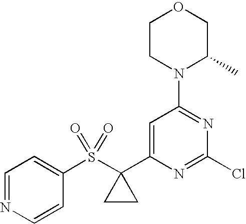

50% v/v Aqueous sodium hydroxide (23 mL, 9.52 mmol) was added to 2-chloro-4-[(3S)-3-methylmorpholin-4-yl]-6-(pyridin-4-ylsulfonylmethyl)pyrimidine (3.51 g, 9.52 mmol), 1,2-dibromoethane (0.820 mL, 9.52 mmol) and tetrabutylammonium bromide (0.307 g, 0.95 mmol) in DCM (100 mL) and the reaction warmed to 30° C. under air. The resulting slurry was stirred at 30° C. for 4 hours then allowed to cool, DCM added and the layers separated. The organic layer was washed with water, dried (MgSO₄) and filtered. The resulting solution was evaporated on to silica and purified by flash silica chromatography, eluting with 0 to 60% ethyl acetate in DCM, to give the desired material as a yellow solid (1.07 g).

NMR Spectrum: ¹H NMR (400.132 MHz, DMSO-d₆) δ 1.15-1.16 (3H, d), 1.61-1.65 (2H, m), 1.90-1.93 (2H, m), 3.11-3.19 (1H, td), 3.37-3.44 (1H, td), 3.53-3.57 (1H, dd), 3.68-3.71 (1H, d), 3.89-3.96 (1H, dd), 3.96 (1H, bs), 4.28 (1H, bs), 6.75 (1H, s), 7.74-7.75 (2H, dd), 8.88-8.90 (2H, dd).

LCMS Spectrum: m/z (ES+) (M+H)+=395; HPLC tR=1.65 min.

### 2-Chloro-4-[(3S)-3-methylmorpholin-4-yl]-6-(pyridin-4-ylsulfonylmethyl)pyrimidine

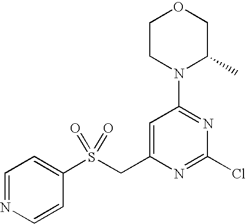

A solution of hydrogen peroxide (1.799 mL, 58.19 mmol) was added dropwise to a stirred solution of 2-chloro-4-[(3S)-3-methylmorpholin-4-yl]-6-(pyridin-4-ylsulfanylmethyl)pyrimidine (0.980 g, 2.91 mmol), sodium tungstate dihydrate (0.005 mL, 0.06 mmol) and 2N sulfuric acid (0.075 mL) in dioxane (200 mL) at 55° C., over a period of 5 minutes under air. The resulting solution was stirred at 55° C. for 3 hours. Water (200 mL) was added and the reaction was cooled, the solids filtered, washed with water and dried in the vacuum oven at 50° C. overnight to give the desired material as a white solid (0.580 g). Additional material was obtained by extracting the aqueous layer with DCM. The extracts were dried (MgSO₄), filtered, evaporated and chromatographed on silica, eluting with 0-3% methanol in DCM, to give a further portion of the desired material (0.144 g).

NMR Spectrum: ¹H NMR (400.13 MHz, DMSO-d₆) δ 1.17-1.19(3H, d), 3.14-3.22(1H, td), 3.40-3.47(1H, td), 3.56-3.60(1H, dd), 3.71-3.74(1H, d), 3.90(1H, bs), 3.91-3.95(1H, dd), 4.20(1H, bs), 4.79(2H, s), 6.79(1H, s), 7.77-7.79(2H, q), 8.92-8.93(2H, q).

LCMS Spectrum: m/z (ES+) (M+H)+=369; HPLC tR=1.40 min.

### 2-Chloro-4-[(3S)-3-methylmorpholin-4-yl]-6-(pyridin-4-ylsulfanylmethyl)pyrimidine

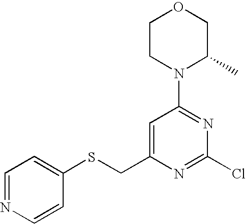

4-Mercaptopyridine (0.752 g, 6.77 mmol) was added to 2-chloro-4-(iodomethyl)-6-[(3S)-3-methylmorpholin-4-yl]pyrimidine (1.596 g, 4.51 mmol) in acetonitrile (100 mL) at RT under air. DBU (0.3 mL, 2.01 mmol) was then added and the resulting solution was stirred at RT for 2 minutes. The solvent was removed and DCM was added. The reaction mixture was washed sequentially with water, the organic layer dried (MgSO₄), filtered and evaporated. The crude product was chromatographed on silica, eluting with 0-2% methanol in DCM. Impure fractions were further chromatographed on silica, eluting with 0-4.5% methanol in DCM and combined with the initial pure fractions to give the desired material as a yellow gum (0.980 g).

NMR Spectrum: ¹H NMR (400.13 MHz, DMSO-d₆) δ 1.14-1.16(3H, d), 3.11-3.18(1H, td), 3.37-3.44(1H, td), 3.53-3.57(1H, dd), 3.64-3.67(1H, d), 3.86-3.90(2H, dd), 4.01(2H, s), 4.14(1H, bs), 6.43(1H, s), 7.04-7.06(2H, d), 8.29-8.30(2H, d).

LCMS Spectrum: m/z (ES+) (M+H)+=337; HPLC tR=1.62 min.

The preparation of 2-chloro-4-(iodomethyl)-6-[(3S)-3-methylmorpholin-4-yl]pyrimidine was described earlier

### EXAMPLE 7

### 3-Methyl-1-[4-[4-[(3S)-3-methylmorpholin-4-yl]-6-(1-propan-2-ylsulfonylcyclopropyl)pyrimidin-2-yl]phenyl]urea

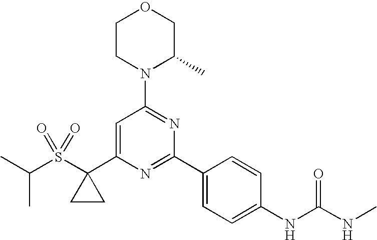

To a solution of phenyl N-[4-[4-[(3S)-3-methylmorpholin-4-yl]-6-(1-propan-2-ylsulfonylcyclopropyl)pyrimidin-2-yl]phenyl]carbamate (120 mg, 0.22 mmol) in DMF (2 mL) was added triethylamine (0.094 mL, 0.67 mmol) followed by methylamine (0.5 mL, 1.1 mmol) and the reaction heated at 50° C. for 2 hours. The crude product was purified by preparative HPLC using decreasingly polar mixtures of water (containing 1% NH3) and acetonitrile as eluents, to give the desired material as a white solid (71 mg).

NMR Spectrum: ¹H NMR (400.132 MHz, DMSO-d₆) δ 1.23 (3H, d), 1.34 (6H, dd), 1.55-1.62 (4H, m), 2.66 (3H, d), 3.17-3.24 (1H, m), 3.48 (1H, dt), 3.60-3.67 (2H, m), 3.76 (1H, d), 3.98 (1H, dd), 4.19 (1H, d), 4.53 (1H, s), 6.07 (1H, d), 6.79 (1H, s), 7.50 (2H, d), 8.18 (2H, d), 8.74 (1H, s)

LCMS Spectrum: m/z (ESI+)(M+H)+=474; HPLC tR=1.92 min.

mTOR Kinase Assay (Echo): 0.00315 μM

The compounds below were prepared in an analogous fashion from phenyl N-[4-[4-[(3S)-3-methylmorpholin-4-yl]-6-(1-propan-2-ylsulfonylcyclopropyl)pyrimidin-2-yl]phenyl]carbamate using the appropriate amine.

| | | | LCMS | Retention |
|---|---|---|---|---|
| Example | Structure | NAME | MH+ | time (min) |
| 7a | 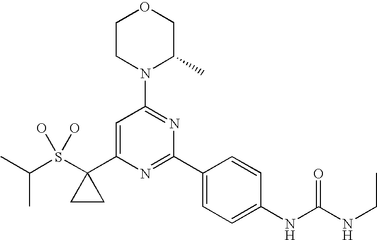 | 1-ethyl-3-[4-[4-[(3S)-3-methylmorpholin-4-yl]-6-(1-propan-2-ylsulfonylcyclopropyl)pyrimidin-2-yl]phenyl]urea | 488 | 1.92 |
| 7b | 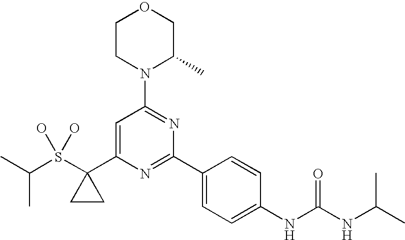 | 1-[4-[4-[(3S)-3-methylmorpholin-4-yl]-6-(1-propan-2-ylsulfonylcyclopropyl)pyrimidin-2-yl]phenyl]-3-propan-2-yl-urea | 502 | 2.08 |
| 7c | 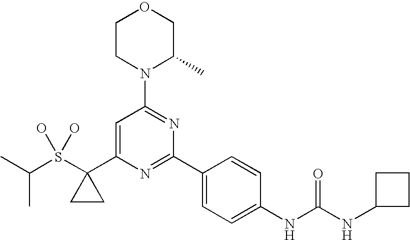 | 3-cyclobutyl-1-[4-[4-[(3S)-3-methylmorpholin-4-yl]-6-(1-propan-2-ylsulfonylcyclopropyl)pyrimidin-2-yl]phenyl]urea | 514 | 2.24 |
| 7d | 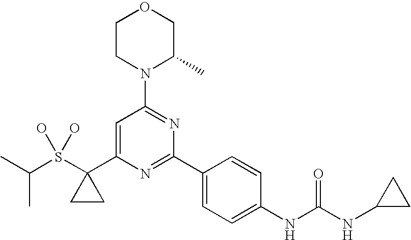 | 3-cyclopropyl-1-[4-[4-[(3S)-3-methylmorpholin-4-yl]-6-(1-propan-2-ylsulfonylcyclopropyl)pyrimidin-2-yl]phenyl]urea | 500 | 2.34 |
| 7e | 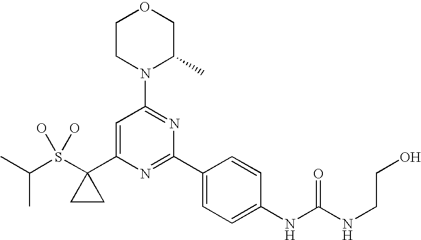 | 3-(2-hydroxyethyl)-1-[4-[4-[(3S)-3-methylmorpholin-4-yl]-6-(1-propan-2-ylsulfonylcyclopropyl)pyrimidin-2-yl]phenyl]urea | 504 | 2.09 |
| 7f | 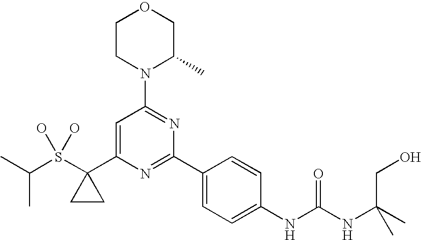 | 3-(1-hydroxy-2-methyl-propan-2-yl)-1-[4-[4-[(3S)-3-methylmorpholin-4-yl]-6-(1-propan-2-ylsulfonylcyclopropyl)pyrimidin-2-yl]phenyl]urea | 532 | 1.77 |
| 7g | 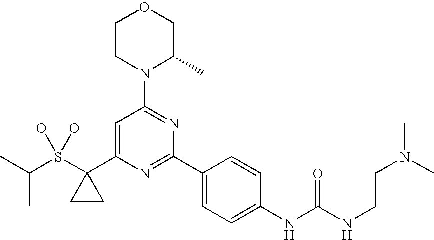 | 3-(2-dimethylaminoethyl)-1-[4-[4-[(3S)-3-methylmorpholin-4-yl]-6-(1-propan-2-ylsulfonylcyclopropyl)pyrimidin-2-yl]phenyl]urea | 531 | 2.06 |
| 7h | 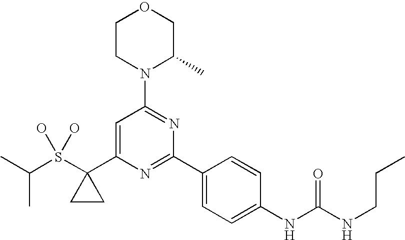 | 3-[4-[4-[(3S)-3-methylmorpholin-4-yl]-6-(1-propan-2-ylsulfonylcyclopropyl)pyrimidin-2-yl]phenyl]-1-propyl-urea | 502 | 2.00 |
| 7i | 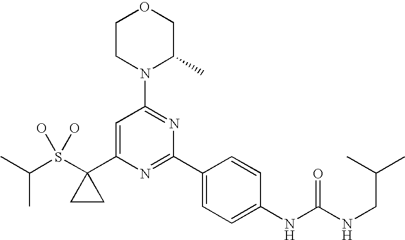 | 1-[4-[4-[(3S)-3-methylmorpholin-4-yl]-6-(1-propan-2-ylsulfonylcyclopropyl)pyrimidin-2-yl]phenyl]-3-(2-methylpropyl)urea | 516 | 2.26 |
| 7j | 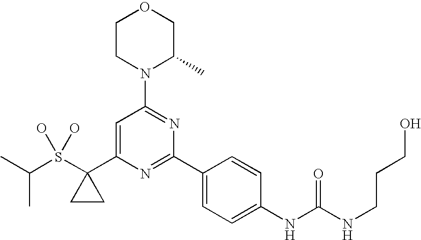 | 3-(3-hydroxypropyl)-1-[4-[4-[(3S)-3-methylmorpholin-4-yl]-6-(1-propan-2-ylsulfonylcyclopropyl)pyrimidin-2-yl]phenyl]urea | 518 | 2.43 |
| 7k | 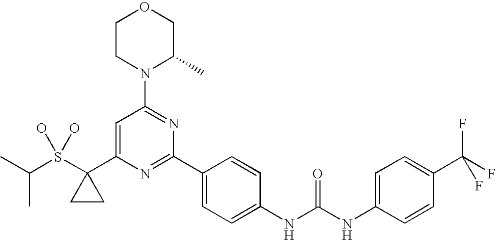 | 1-[4-[4-[(3S)-3-methylmorpholin-4-yl]-6-(1-propan-2-ylsulfonylcyclopropyl)pyrimidin-2-yl]phenyl]-3-[4-(trifluoromethyl)phenyl]urea | 604 | 1.81 |
| 7l | 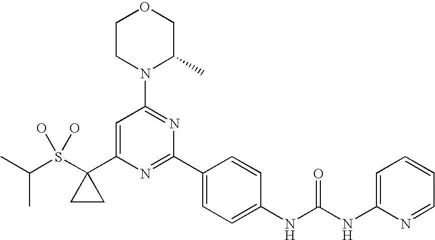 | 1-[4-[4-[(3S)-3-methylmorpholin-4-yl]-6-(1-propan-2-ylsulfonylcyclopropyl)pyrimidin-2-yl]phenyl]-3-pyridin-2-yl-urea | 537 | 2.95 |
| 7m | 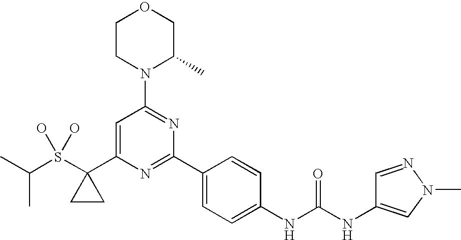 | 1-[4-[4-[(3S)-3-methylmorpholin-4-yl]-6-(1-propan-2-ylsulfonylcyclopropyl)pyrimidin-2-yl]phenyl]-3-(1-methylpyrazol-4-yl)urea | 540 | 2.54 |

### EXAMPLE 7a

¹H NMR (400.132 MHz, DMSO-d₆) δ 1.07 (3H, t), 1.23 (3H, d), 1.34 (6H, dd), 1.52-1.61 (4H, m), 3.09-3.16 (2H, m), 3.16-3.23 (1H, m), 3.49 (1H, dt), 3.62-3.67 (2H, m), 3.76 (1H, d), 3.97 (1H, d), 4.19 (1H, d), 4.53 (1H, s), 6.17 (1H, t), 6.79 (1H, s), 7.50 (2H, d), 8.18 (2H, d), 8.66 (1H, s).

mTOR Kinase Assay (Echo): 0.00231 μM

### EXAMPLE 7b

¹H NMR (400.132 MHz, DMSO-d₆) δ 1.11 (6H, d), 1.23 (3H, d), 1.34 (6H, dd), 1.52-1.61 (4H, m), 3.15-3.24 (1H, m), 3.49 (1H, dt), 3.60-3.67 (2H, m), 3.73-3.82 (2H, m), 3.97 (1H, dd), 4.19 (1H, d), 4.53 (1H, s), 6.07 (1H, d), 6.79 (1H, s), 7.48 (2H, d), 8.18 (2H, d), 8.53 (1H, s).

mTOR Kinase Assay (Echo): 0.0181 μM

### EXAMPLE 7c

¹H NMR (400.132 MHz, DMSO-d₆) δ 1.23 (3H, d), 1.33 (3H, d), 1.35 (3H, d), 1.52-1.66 (6H, m), 1.81-1.91 (2H, m), 2.18-2.25 (2H, m), 3.20 (1H, dt), 3.48 (1H, dt), 3.60-3.67 (2H, m), 3.76 (1H, d), 3.97 (1H, dd), 4.09-4.21 (2H, m), 4.53 (1H, s), 6.47 (1H, d), 6.79 (1H, s), 7.48 (2H, d), 8.18 (2H, d), 8.56 (1H, s).

mTOR Kinase Assay (Echo): 0.00646 μM

### EXAMPLE 7d

¹H NMR (400.132 MHz, DMSO-d₆) δ 0.40-0.44 (2H, m), 0.63-0.67 (2H, m), 1.23 (3H, d), 1.33 (3H, d), 1.35 (3H, d), 1.53-1.62 (4H, m), 2.54-2.58 (1H, m), 3.17-3.24 (1H, m), 3.49 (1H, dt), 3.60-3.67 (2H, m), 3.76 (1H, d), 3.97 (1H, dd), 4.20 (1H, d), 4.53 (1H, s), 6.44 (1H, d), 6.80 (1H, s), 7.51 (2H, d), 8.19 (2H, d), 8.53 (1H, s).

mTOR Kinase Assay (Echo): 0.0038 μM

### EXAMPLE 7e

¹H NMR (400.132 MHz, DMSO-d₆) δ 1.23 (3H, d), 1.33 (3H, d), 1.35 (3H, d), 1.54-1.61 (4H, m), 3.16-3.24 (3H, m), 3.44-3.52 (3H, m), 3.60-3.67 (2H, m), 3.76 (1H, d), 3.97 (1H, dd), 4.19 (1H, d), 4.52 (1H, s), 4.73 (1H, t), 6.26 (1H, t), 6.79 (1H, s), 7.50 (2H, d), 8.19 (2H, d), 8.80 (1H, s).

mTOR Kinase Assay (Echo): 0.00212 μM

### EXAMPLE 7f

¹H NMR (400.132 MHz, DMSO-d₆) δ 1.23 (3H, d), 1.24 (6H, s), 1.34 (3H, d), 1.35 (3H, d), 1.55-1.62 (4H, m), 3.15-3.23 (1H, m), 3.39 (2H, d), 3.49 (1H, dt), 3.62-3.68 (2H, m), 3.76 (1H, d), 3.97 (1H, d), 4.20 (1H, d), 4.52 (1H, s), 4.95 (1H, t), 6.01 (1H, s), 6.79 (1H, s), 7.46 (2H, d), 8.18 (2H, d), 8.73 (1H, s).

mTOR Kinase Assay (Echo): 0.00915 μM

### EXAMPLE 7g

¹H NMR (400.132 MHz, DMSO-d₆) δ 1.23 (3H, d), 1.33 (3H, d), 1.35 (3H, d), 1.52-1.61 (4H, m), 2.18 (6H, s), 2.34 (2H, t), 3.17-3.24 (3H, m), 3.49 (1H, dt), 3.60-3.67 (2H, m), 3.76 (1H, d), 3.97 (1H, dd), 4.19 (1H, d), 4.52 (1H, s), 6.16 (1H, t), 6.79 (1H, s), 7.49 (2H, d), 8.18 (2H, d), 8.89 (1H, s).

mTOR Kinase Assay (Echo): 0.211 μM

### EXAMPLE 7h

¹H NMR (400.132 MHz, DMSO-d₆) δ 0.89 (3H, t), 1.23 (3H, d), 1.33 (3H, d), 1.35 (3H, d), 1.43-1.49 (2H, m), 1.53-1.62 (4H, m), 3.07 (2H, q), 3.16-3.23 (1H, m), 3.49 (1H, dt), 3.60-3.67 (2H, m), 3.76 (1H, d), 3.97 (1H, d), 4.20 (1H, d), 4.54 (1H, s), 6.21 (1H, t), 6.79 (1H, s), 7.50 (2H, d), 8.19 (2H, d), 8.64 (1H, s).

mTOR Kinase Assay (Echo): 0.0116 μM

### EXAMPLE 7i

¹H NMR (400.132 MHz, DMSO-d₆) δ 0.89 (6H, d), 1.23 (3H, d), 1.33 (3H, d), 1.35 (3H, d), 1.55-1.62 (4H, m), 1.67-1.74 (1H, m), 2.94 (2H, t), 3.18-3.23 (1H, m), 3.46-3.51 (1H, m), 3.60-3.67 (2H, m), 3.76 (1H, d), 3.97 (1H, dd), 4.20 (1H, d), 4.51 (1H, s), 6.25 (1H, t), 6.79 (1H, s), 7.49 (2H, d), 8.19 (2H, d), 8.64 (1H, s).

mTOR Kinase Assay (Echo): 0.0182 μM

### EXAMPLE 7j

¹H NMR (400.132 MHz, DMSO-d₆) δ 1.23 (3H, d), 1.33 (3H, d), 1.35 (3H, d), 1.53-1.63 (4H, m), 3.15-3.23 (4H, m), 3.45-3.51 (4H, m), 3.60-3.67 (2H, m), 3.76 (1H, d), 3.97 (1H, d), 4.19 (1H, d), 4.47 (1H, t), 4.53 (1H, s), 6.21 (1H, t), 6.79 (1H, s), 7.50 (2H, d), 8.18 (2H, d), 8.71 (1H, s).

mTOR Kinase Assay (Echo): 0.0105 μM

### EXAMPLE 7k

¹H NMR (400.132 MHz, DMSO-d₆) δ 1.24 (3H, d), 1.34 (3H, d), 1.36 (3H, d), 1.54-1.64 (4H, m), 3.19-3.25 (1H, m), 3.50 (1H, t), 3.61-3.66 (2H, m), 3.77 (1H, d), 3.98 (1H, d), 4.22 (1H, d), 4.55 (1H, s), 6.82 (1H, s), 7.59 (2H, d), 7.64-7.70 (4H, m), 8.27 (2H, d), 9.04 (1H, s), 9.14 (1H, s).

mTOR Kinase Assay (Echo): 0.00482 μM

### EXAMPLE 7l

¹H NMR (400.132 MHz, DMSO-d₆) δ 1.24 (3H, d), 1.35 (3H, d), 1.37 (3H, d), 1.57-1.61 (4H, m), 3.18-3.27 (1H, m), 3.50 (1H, dt), 3.63-3.70 (2H, m), 3.77 (1H, d), 3.98 (1H, dd), 4.21 (1H, d), 4.55 (1H, s), 6.82 (1H, s), 7.02-7.05 (1H, m), 7.55-7.58 (1H, m), 7.65 (2H, d), 7.77 (1H, t), 8.27-8.31 (3H, m), 9.47 (1H, s), 10.62 (1H, s).

mTOR Kinase Assay (Echo): 0.00913 μM

### EXAMPLE 7m

¹H NMR (400.132 MHz, DMSO-d₆) δ 1.24 (3H, d), 1.34 (3H, d), 1.36 (3H, d), 1.53-1.63 (4H, m), 3.17-3.25 (1H, m), 3.49 (1H, t), 3.61-3.68 (2H, m), 3.77 (1H, d), 3.79 (3H, s), 3.97 (1H, d), 4.20 (1H, d), 4.53 (1H, s), 6.81 (1H, s), 7.38 (1H, s), 7.55 (2H, d), 7.76 (1H, s), 8.22 (2H, d), 8.39 (1H, s), 8.83 (1H, s).

mTOR Kinase Assay (Echo): 0.00504 μM

The preparation of phenyl N-[4-[4-[(3S)-3-methylmorpholin-4-yl]-6-(1-propan-2-ylsulfonylcyclopropyl)pyrimidin-2-yl]phenyl]carbamate is described below.

### Phenyl N-[4-[4-[(3S)-3-methylmorpholin-4-yl]-6-(1-propan-2-ylsulfonylcyclopropyl)pyrimidin-2-yl]phenyl]carbamate

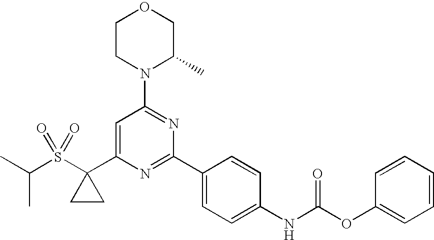

To a solution of 4-[4-[(3S)-3-methylmorpholin-4-yl]-6-(1-propan-2-ylsulfonylcyclopropyl)pyrimidin-2-yl]aniline (1.47 g, 3.53 mmol) in 1,4-dioxane (17.65 mL) was added sodium bicarbonate (0.445 g, 5.29 mmol) and phenyl chloroformate (0.474 mL, 3.77 mmol) and the reaction stirred at RT for 2 hours. The reaction mixture was diluted with DCM (20 mL), and washed with water (20 mL), the organic layer dried (MgSO₄), filtered and evaporated. The crude solid was triturated with diethyl ether to give a solid which was collected by filtration and dried under vacuum to give the desired product as a white solid (1.56 g).

NMR Spectrum: ¹H NMR (400.132 MHz, DMSO-d₆) δ 1.17 (3H, d), 1.27 (3H, d), 1.28 (3H, d), 1.49-1.55 (4H, m), 3.14 (1H, dt), 3.39-3.44 (1H, m), 3.53-3.60 (2H, m), 3.70 (1H, d), 3.90 (1H, dd), 4.14 (1H, d), 4.47 (1H, s), 6.77 (1H, s), 7.17-7.23 (3H, m), 7.38 (2H, t), 7.57 (2H, d), 8.22 (2H, d), 10.37 (1H, s)

LCMS Spectrum: m/z (ESI+)(M+H)+=537; HPLC tR=2.39 min.

### 4-[4-[(3S)-3-Methylmorpholin-4-yl]-6-(1-propan-2-ylsulfonylcyclopropyl)pyrimidin-2-yl]aniline

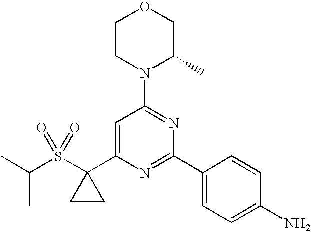

To a solution of 2-chloro-4-[(3S)-3-methylmorpholin-4-yl]-6-(1-propan-2-ylsulfonylcyclopropyl)pyrimidine (1.6 g, 4.45 mmol) in DMF (0.24 mL), DME (9.33 mL), water (4.0 mL) and ethanol (2.67 mL) was added 4-(4,4,5,5-tetramethyl-1,3,2-dioxaborolan-2-yl)aniline (1.266 g, 5.78 mmol), sodium carbonate (5 mL, 10.00 mmol) and dichlorobis(triphenylphosphine)palladium(II) (0.156 g, 0.22 mmol) and the suspension heated at 95° C. for 2 hours. The reaction mixture was cooled to RT, diluted with ethyl acetate (10 mL) and washed with water (2×10 mL). The organic layer was dried (MgSO₄), filtered and evaporated. The crude product was purified by flash silica chromatography, elution gradient 5 to 60% ethyl acetate in isohexane, to give the desired material as a cream solid (1.47 g).

NMR Spectrum: ¹H NMR (400.132 MHz, DMSO-d₆) δ 1.21 (3H, d), 1.32 (3H, d), 1.34 (3H, d), 1.50-1.59 (4H, m), 3.17 (1H, dt), 3.44-3.51 (1H, m), 3.59-3.66 (2H, m), 3.75 (1H, d), 3.96 (1H, dd), 4.16 (1H, d), 4.49 (1H, d), 5.56 (2H, s), 6.61 (2H, d), 6.69 (1H, s), 8.02 (2H, d)

LCMS Spectrum: m/z (ESI+) (M+H)+=417; HPLC tR=2.09 min.

### 2-Chloro-4-[(3S)-3-methylmorpholin-4-yl]-6-(1-propan-2-ylsulfonylcyclopropyl)pyrimidine

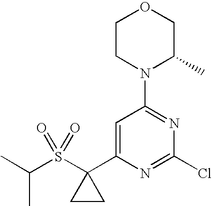

2-Chloro-4-[(3S)-3-methylmorpholin-4-yl]-6-(propan-2-ylsulfonylmethyl)pyrimidine (2.4 g, 7.19 mmol) was dissolved in DCM (40 mL) and sodium hydroxide concentrate (7.2 mL, 71.89 mmol) was added to the reaction, followed by dibromoethane (0.325 mL, 14.38 mmol). The reaction was stirred at 40° C. for 10 hours. The reaction mixture was washed with water (50 mL) and the organic layer dried (MgSO₄), filtered and evaporated to afford crude product. The crude product was purified by flash silica chromatography, elution gradient 0 to 50% ethyl acetate in DCM, to give the desired material as a white solid (1.49 g).

NMR Spectrum: ¹H NMR (400.132 MHz, DMSO-d₆) δ 1.21 (3H, d), 1.27 (6H, d), 1.52-1.54 (2H, m), 1.56-1.59 (2H, m), 3.21-3.24 (1H, m), 3.41-3.47 (1H, m), 3.55-3.61 (2H, m), 3.72 (1H, d), 3.93 (1H, dd), 4.02 (1H, d), 4.37 (1H, s), 6.94 (1H, s)

LCMS Spectrum: m/z (ESI+)(M+H)+ 360, HPLC tR=1.89 min

### 2-Chloro-4-[(3S)-3-methylmorpholin-4-yl]-6-(propan-2-ylsulfonylmethyl)pyrimidine

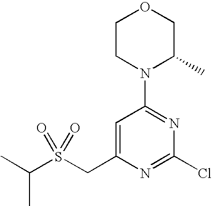

2,4-Dichloro-6-[(isopropylsulfonyl)methyl]pyrimidine (2.65 g, 9.85 mmol) was dissolved in DCM (50 mL) and stirred (under nitrogen) at −5° C. Triethylamine (1.5 mL, 10.84 mmol) was added to give a clear brown solution. (3S)-3-Methyl morpholine (997 mg, 9.85 mmol) was dissolved in DCM and added dropwise keeping the reaction below −5° C. The cooling bath was then removed and the reaction mixture stirred at room temperature for 1 hour. The reaction mixture was then washed with water (50 mL), dried over magnesium sulphate, filtered and concentrated in vacuo. The crude material was chromatographed on silica, eluting with 0-50% ethyl acetate in DCM to give the desired material as a white solid (2 g).

NMR Spectrum: ¹H NMR (400.132 MHz, DMSO-d₆) δ 1.22 (d, 3H), 1.31 (d, 6H), 3.22 (m, 1H), 3.43 (m, 2H), 3.60 (m, 1H), 3.74 (d, 1H), 3.98 (m, 1H), 4.30 (s, 1H), 4.43 (s, 2H), 6.91 (s, 1H)

LCMS Spectrum: m/z (ESI+)(M+H)+ 332, HPLC tR=1.70 min

### 2,4-Dichloro-6-[(isopropylsulfonyl)methyl]pyrimidine

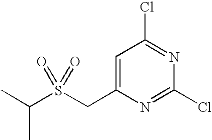

2,4-Dichloro-6-[(isopropylthio)methyl]pyrimidine (6.2 g, 26.16 mmol) was dissolved in DCM (100 mL) and 3,5-dichlorobenzenecarboperoxoic acid (13.5 g, 78.4 mmol) was added portionwise over 10 minutes. The reaction was stirred at room temperature for 4 hours. The reaction mixture was then washed with saturated aqueous sodium bicarbonate (50 mL), dried over magnesium sulphate, filtered and concentrated in vacuo to give a cream solid.

Purification by normal phase chromatography, eluting with 0-50% ethyl acetate in iso-hexane gave the desired material as a cream solid (5.3 g).

NMR Spectrum: ¹H NMR (400.132 MHz, DMSO-d₆) δ 1.25 (d, 6H), 3.43 (m, 1H), 4.77 (s, 2H), 7.87 (s, 1H)

LCMS Spectrum: m/z (M−H)⁻ 267, HPLC tR=1.64 min

### 2,4-Dichloro-6-[(isopropylthio)methyl]pyrimidine

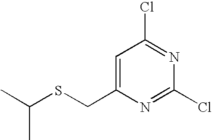

6-[(Isopropylthio)methyl]pyrimidine-2,4(1H,3H)-dione (8 g, 40 mmol) was added to phosphorus oxychloride (100 mL) and the mixture heated to reflux for 16 hours. The reaction was then cooled to room temperature and the excess phosphorus oxychloride was removed in vacuo. The residue was azeotroped with toluene (2×100 mL) and dissolved in DCM. This mixture was then poured slowly onto ice (1 L) and stirred for 20 minutes, then extracted with DCM (3×500 mL) The extracts were combined, dried over magnesium sulphate, then concentrated in vacuo to give the desired material as a brown oil (6.5 g). The material was used without further purification.

NMR Spectrum: ¹H NMR (400.132 MHz, DMSO-d₆) δ 1.21 (d, 6H), 2.96 (m, 1H), 3.85 (s, 2H), 7.82 (s, 1H)

LCMS Spectrum: No mass ion observed, HPLC tR=2.51 min

### 6-[(Isopropylthio)methyl]pyrimidine-2,4(1H,3H)-dione

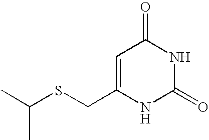

6-(Chloromethyl)-1H-pyrimidine-2,4-dione (8 g, 50 mmol) was dissolved in acetonitrile (200 mL) and 1,8-Diazabicyclo[5.4.0]undec-7-ene (13 mL, 87.19 mmol) was added and the reaction stirred at room temperature for 15 minutes. Isopropyl mercaptan (8.1 mL, 87.19 mmol) was then added and the reaction stirred at room temperature for a further 2 hours. Solvent removed in vacuo and the resulting brown oil was dissolved in DCM and washed with water. Organic phase dried over magnesium sulphate, filtered and concentrated in vacuo. The resulting oil was chromatographed on silica, eluting with 0-10% methanol in DCM to give the desired material as a white solid (8 g).

NMR Spectrum: ¹H NMR (400.132 MHz, DMSO-d₆) δ 1.21 (d, 6H), 2.90 (m, 1H), 3.42 (s, 2H), 5.49 (s, 1H), 10.82 (s, 1H), 10.94 (s, 1H)

LCMS Spectrum: m/z (M−H)⁻ 199, HPLC tR=0.63 min

### EXAMPLE 8

### 1-[4-[4-[1-(4-Fluorophenyl)sulfonylcyclopropyl]-6-[(3S)-3-methylmorpholin-4-yl]pyrimidin-2-yl]phenyl]-3-methyl-urea

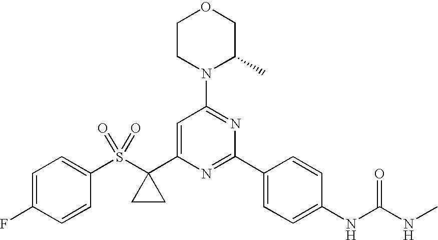

To a solution of phenyl N-[4-[4-[1-(4-fluorophenyl)sulfonylcyclopropyl]-6-[(3S)-3-methylmorpholin-4-yl]pyrimidin-2-yl]phenyl]carbamate (100 mg, 0.17 mmol) in DMF (2 mL) was added triethylamine (0.071 mL, 0.51 mmol) followed by methylamine (0.5 mL, 1.1 mmol) and the reaction heated at 50° C. for 2 hours. The crude product was purified by preparative HPLC using decreasingly polar mixtures of water (containing 1% NH3) and acetonitrile as eluents, to give the desired material as a white solid (51 mg).

NMR Spectrum: ¹H NMR (400.132 MHz, DMSO-d₆) δ 1.18 (3H, d), 1.59-1.62 (2H, m), 1.88-1.90 (2H, m), 2.66 (3H, d), 3.15 (1H, dt), 3.46 (1H, dt), 3.61 (1H, dd), 3.75 (1H, d), 3.96 (1H, dd), 4.13 (1H, d), 4.42 (1H, s), 6.03 (1H, d), 6.65 (1H, s), 7.38-7.44 (4H, m), 7.79-7.86 (4H, m), 8.71 (1H, s)

LCMS Spectrum: m/z (ESI+)(M+H)+=526; HPLC tR=2.09 min.

mTOR Kinase Assay (Echo): 0.000576 μM

The following compounds were prepared in an analogous fashion from phenyl N-[4-[4-[1-(4-fluorophenyl)sulfonylcyclopropyl]-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 8a | 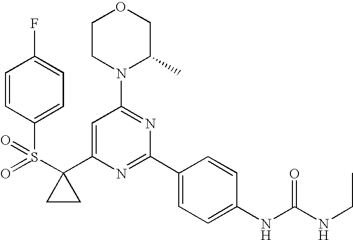 | 1-ethyl-3-[4-[4-[1-(4-fluorophenyl)sulfonylcyclopropyl]-6-[(3S)-3-methylmorpholin-4-yl]pyrimidin-2-yl]phenyl]urea | 540 | 2.23 |
| 8b | 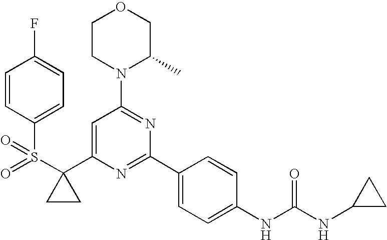 | 3-cyclopropyl-1-[4-[4-[1-(4-fluorophenyl)sulfonylcyclopropyl]-6-[(3S)-3-methylmorpholin-4-yl]pyrimidin-2-yl]phenyl]urea | 552 | 2.25 |
| 8c | 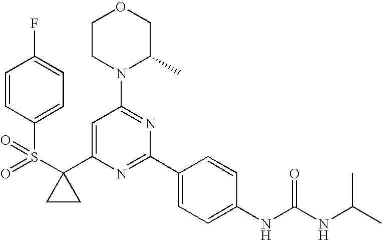 | 1-[4-[4-[1-(4-fluorophenyl)sulfonylcyclopropyl]-6-[(3S)-3-methylmorpholin-4-yl]pyrimidin-2-yl]phenyl]-3-propan-2-yl-urea | 554 | 2.40 |
| 8d | 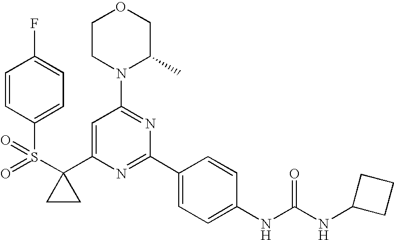 | 3-cyclobutyl-1-[4-[4-[1-(4-fluorophenyl)sulfonylcyclopropyl]-6-[(3S)-3-methylmorpholin-4-yl]pyrimidin-2-yl]phenyl]urea | 566 | 2.47 |
| 8e | 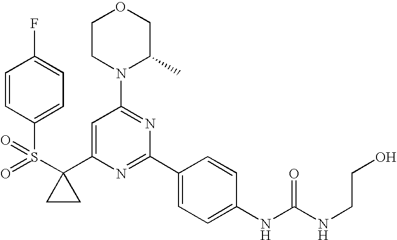 | 1-[4-[4-[1-(4-fluorophenyl)sulfonylcyclopropyl]-6-[(3S)-3-methylmorpholin-4-yl]pyrimidin-2-yl]phenyl]-3-(2-hydroxyethyl)urea | 556 | 1.92 |
| 8f | 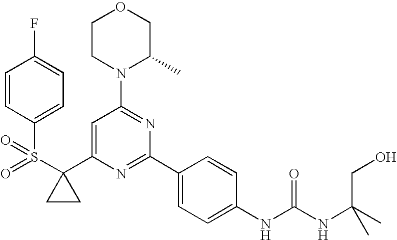 | 1-[4-[4-[1-(4-fluorophenyl)sulfonylcyclopropyl]-6-[(3S)-3-methylmorpholin-4-yl]pyrimidin-2-yl]phenyl]-3-(1-hydroxy-2-methyl-propan-2-yl)urea | 584 | 2.22 |
| 8g | 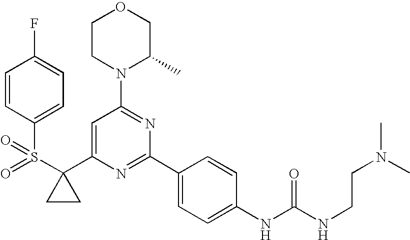 | 3-(2-dimethylaminoethyl)-1-[4-[4-[1-(4-fluorophenyl)sulfonylcyclopropyl]-6-[(3S)-3-methylmorpholin-4-yl]pyrimidin-2-yl]phenyl]urea | 583 | 2.17 |
| 8h | 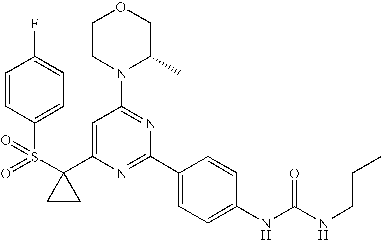 | 3-[4-[4-[1-(4-fluorophenyl)sulfonylcyclopropyl]-6-[(3S)-3-methylmorpholin-4-yl]pyrimidin-2-yl]phenyl]-1-propyl-urea | 554 | 2.40 |
| 8i | 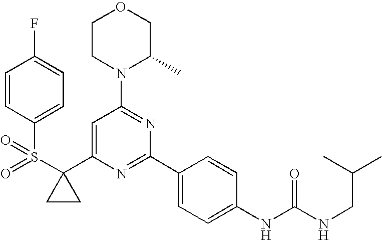 | 1-[4-[4-[1-(4-fluorophenyl)sulfonylcyclopropyl]-6-[(3S)-3-methylmorpholin-4-yl]pyrimidin-2-yl]phenyl]-3-(2-methylpropyl)urea | 568 | 2.55 |
| 8j | 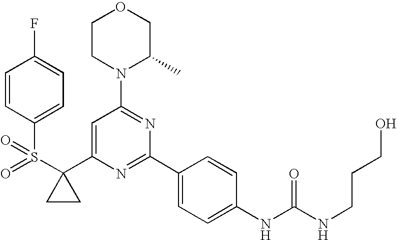 | 1-[4-[4-[1-(4-fluorophenyl)sulfonylcyclopropyl]-6-[(3S)-3-methylmorpholin-4-yl]pyrimidin-2-yl]phenyl]-3-(3-hydroxypropyl)urea | 570 | 1.97 |
| 8k | 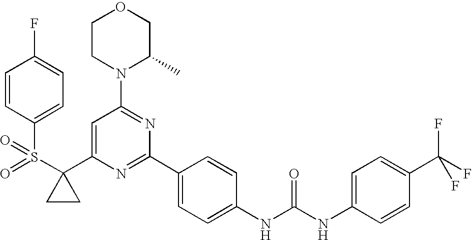 | 1-[4-[4-[1-(4-fluorophenyl)sulfonylcyclopropyl]-6-[(3S)-3-methylmorpholin-4-yl]pyrimidin-2-yl]phenyl]-3-[4-(trifluoromethyl)phenyl]urea | 656 | 3.01 |
| 8l | 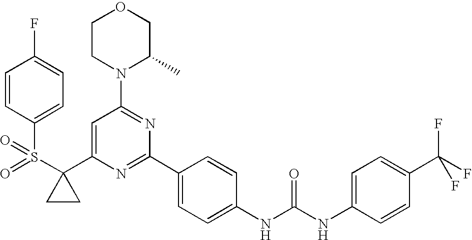 | 1-[4-[4-[1-(4-fluorophenyl)sulfonylcyclopropyl]-6-[(3S)-3-methylmorpholin-4-yl]pyrimidin-2-yl]phenyl]-3-pyridin-2-yl-urea | 589 | 2.63 |
| 8m | 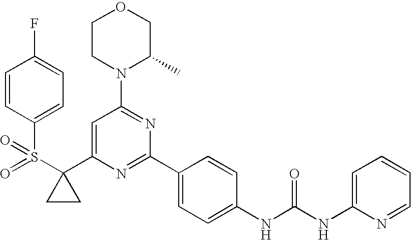 | 1-[4-[4-[1-(4-fluorophenyl)sulfonylcyclopropyl]-6-[(3S)-3-methylmorpholin-4-yl]pyrimidin-2-yl]phenyl]-3-(1-methylpyrazol-4-yl)urea | 592 | 2.12 |

Example 8 can also be prepared in an analogous fashion to that described above but using NMP as the solvent and stirring at 75° C. for 30 minutes. The material can then be partitioned between ethyl acetate and water and the organic materials purified by chromatography on silica, eluting with 0-3% methanol in ethyl acetate. The material can then be dissolved in DCM and either evapourated rapidly to give the desired material as a foam or left to stand for approximately 6 weeks upon which time the desired material precipitated from solution.

### EXAMPLE 8a

¹H NMR (400.132 MHz, DMSO-d₆) δ 1.06 (3H, t), 1.18 (3H, d), 1.59-1.62 (2H, m), 1.88-1.90 (2H, m), 3.09-3.18 (3H, m), 3.46 (1H, dt), 3.61 (1H, d), 3.95 (1H, dd), 4.13 (1H, d), 4.42 (1H, s), 6.12 (1H, t), 6.65 (1H, s), 7.37-7.46 (4H, m), 7.79-7.86 (4H, m), 8.63 (1H, s).

mTOR Kinase Assay (Echo): 0.00096 μM

### EXAMPLE 8b

¹H NMR (400.132 MHz, DMSO-d₆) δ 0.40-0.43 (2H, m), 0.62-0.67 (2H, m), 1.19 (3H, d), 1.58-1.63 (2H, m), 1.89-1.91 (2H, m), 2.55-2.58 (1H, m), 3.15 (1H, t), 3.46 (1H, t), 3.61 (1H, d), 3.75 (1H, d), 3.96 (1H, d), 4.14 (1H, d), 4.42 (1H, s), 6.39 (1H, s), 6.65 (1H, s), 7.39-7.44 (4H, m), 7.80-7.86 (4H, m), 8.51 (1H, s).

mTOR Kinase Assay (Echo): 0.00123 μM

### EXAMPLE 8c

¹H NMR (400.132 MHz, DMSO-d₆) δ 1.11 (6H, d), 1.18 (3H, d), 1.58-1.62 (2H, m), 1.88-1.90 (2H, m), 3.15 (1H, t), 3.46 (1H, t), 3.61 (1H, d), 3.73-3.81 (2H, m), 3.96 (1H, d), 4.13 (1H, d), 4.42 (1H, s), 6.02 (1H, d), 6.65 (1H, s), 7.37 (2H, d), 7.42 (2H, t), 7.29-7.86 (4H, m), 8.51 (1H, s).

mTOR Kinase Assay (Echo): 0.00185 μM

### EXAMPLE 8d

¹H NMR (400.132 MHz, DMSO-d₆) δ 1.18 (3H, d), 1.59-1.66 (4H, m), 1.83-1.91 (4H, m), 2.18-2.26 (2H, m), 3.12-3.19 (1H, m), 3.46 (1H, t), 3.61 (1H, d), 3.74 (1H, d), 3.95 (1H, d), 4.11-4.17 (2H, m), 4.42 (1H, s), 6.42 (1H, d), 6.65 (1H, s), 7.37 (2H, d), 7.42 (2H, t), 7.79-7.86 (4H, m), 8.54 (1H, s).

mTOR Kinase Assay (Echo): 0.00134 μM

### EXAMPLE 8e

¹H NMR (400.132 MHz, DMSO-d₆) δ 1.18 (3H, d), 1.59-1.62 (2H, m), 1.88-1.90 (2H, m), 3.15-3.18 (2H, m), 3.40-3.48 (2H, m), 3.61 (2H, dd), 3.74 (1H, d), 3.95 (1H, d), 4.05-4.16 (2H, m), 4.13 (1H, d), 4.73 (1H, t), 6.22 (1H, t), 6.64 (1H, s), 7.37-7.44 (4H, m), 7.79-7.86 (4H, m), 8.78 (1H, s).

mTOR Kinase Assay (Echo): 0.000342 μM

### EXAMPLE 8f

¹H NMR (400.132 MHz, DMSO-d₆) δ 1.18 (3H, d), 1.24 (6H, s), 1.57-1.63 (2H, m), 1.86-1.91 (2H, m), 3.10-3.18 (1H, m), 3.38 (2H, d), 3.46 (1H, t), 3.61 (1H, d), 3.75 (1H, d), 3.96 (1H, d), 4.14 (1H, d), 4.41 (1H, s), 4.95 (1H, t), 5.97 (1H, s), 6.64 (1H, s), 7.34 (2H, d), 7.42 (2H, t), 7.78-7.86 (4H, m), 8.71 (1H, s).

mTOR Kinase Assay (Echo): 0.00882 μM

### EXAMPLE 8g

¹H NMR (400.132 MHz, DMSO-d₆) δ 1.18 (3H, d), 1.59-1.62 (2H, m), 1.88-1.90 (2H, m), 2.18 (6H, s), 2.33 (2H, t), 3.15-3.21 (3H, m), 3.46 (1H, dt), 3.61 (1H, dd), 3.75 (1H, d), 3.96 (1H, dd), 4.13 (1H, d), 4.42 (1H, s), 6.13 (1H, t), 6.65 (1H, s), 7.37-7.43 (4H, m), 7.79-7.86 (4H, m), 8.86 (1H, s).

mTOR Kinase Assay (Echo): 0.0613 μM

### EXAMPLE 8h

¹H NMR (400.132 MHz, DMSO-d₆) δ 0.89 (3H, t), 1.18 (3H, d), 1.45 (2H, sextet), 1.59-1.62 (2H, m), 1.88-1.90 (2H, m), 3.06 (2H, q), 3.12-3.16 (1H, m), 3.40-3.19 (1H, m), 3.61 (1H, dd), 3.74 (1H, d), 3.96 (1H, dd), 4.13 (1H, d), 4.42 (1H, s), 6.17 (1H, t), 6.64 (1H, s), 7.37-7.44 (4H, m), 7.79-7.86 (4H, m), 8.63 (1H, s).

mTOR Kinase Assay (Echo): 0.00176 μM

### EXAMPLE 8i

¹H NMR (400.132 MHz, DMSO-d₆) δ 0.88 (6H, d), 1.19 (3H, d), 1.59-1.62 (2H, m), 1.67-1.74 (1H, m), 1.88-1.90 (2H, m), 2.94 (2H, t), 3.12-3.19 (1H, m), 3.46 (1H, dt), 3.61 (1H, dd), 3.75 (1H, d), 3.96 (1H, dd), 4.14 (1H, d), 4.42 (1H, s), 6.21 (1H, t), 6.65 (1H, s), 7.37-7.43 (4H, m), 7.79-7.85 (4H, m), 8.62 (1H, s).

mTOR Kinase Assay (Echo): 0.0198 μM

### EXAMPLE 8j

¹H NMR (400.132 MHz, DMSO-d₆) δ 1.18 (3H, d), 1.56-1.63 (4H, m), 1.88-1.90 (2H, m), 3.12-3.19 (3H, m), 3.47 (2H, q), 3.61 (1H, dd), 3.75 (1H, d), 3.96 (1H, dd), 4.13 (1H, d), 4.43 (1H, s), 4.47 (2H, t), 6.17 (1H, t), 6.65 (1H, s), 7.37-7.45 (4H, m), 7.79-7.86 (4H, m), 8.68 (1H, s).

mTOR Kinase Assay (Echo): 0.000751 μM

### EXAMPLE 8k

¹H NMR (400.132 MHz, DMSO-d₆) δ 1.20 (3H, d), 1.60-1.63 (2H, m), 1.89-1.92 (2H, m), 3.13-3.21 (1H, m), 3.47 (1H, dt), 3.62 (1H, dd), 3.75 (1H, d), 3.97 (1H, dd), 4.15 (1H, d), 4.43 (1H, s), 6.68 (1H, s), 7.41-7.49 (4H, m), 7.63-7.69 (4H, m), 7.84-7.89 (4H, m), 9.02 (1H, s), 9.10 (1H, s).

mTOR Kinase Assay (Echo): 0.0173 μM

### EXAMPLE 81

¹H NMR (400.132 MHz, DMSO-d₆) δ 1.20 (3H, d), 1.61-1.63 (2H, m), 1.89-1.92 (2H, m), 3.14-3.21 (1H, m), 3.47 (1H, t), 3.62 (1H, d), 3.76 (1H, d), 3.96 (1H, d), 4.16 (1H, d), 4.44 (1H, s), 6.68 (1H, s), 7.02-7.05 (1H, m), 7.43 (2H, t), 7.53 (2H, d), 7.57-7.62 (1H, m), 7.75-7.79 (1H, m), 7.83-7.91 (4H, m), 8.30 (1H, d), 9.40 (1H, s), 10.50 (1H, s).

mTOR Kinase Assay (Echo): 0.00813 μM

### EXAMPLE 8m

¹H NMR (400.132 MHz, DMSO-d₆) δ 1.19 (3H, d), 1.60-1.62 (2H, m), 1.89-1.91 (2H, m), 3.12-3.20 (1H, m), 3.47 (1H, dt), 3.62 (1H, dd), 3.75 (1H, d), 3.79 (3H, s), 3.96 (1H, dd), 4.14 (1H, d), 4.43 (1H, s), 6.66 (1H, s), 7.38-7.45 (5H, m), 7.77 (1H, s), 7.83-7.87 (4H, m), 8.35 (1H, s), 8.82 (1H, s).

mTOR Kinase Assay (Echo): 0.00136 μM

The preparation of phenyl N-[4-[4-[1-(4-fluorophenyl)sulfonylcyclopropyl]-6-[(3S)-3-methylmorpholin-4-yl]pyrimidin-2-yl]phenyl]carbamate is described below:

### Phenyl N-[4-[4-[1-(4-fluorophenyl)sulfonylcyclopropyl]-6-[(3S)-3-methylmorpholin-4-yl]pyrimidin-2-yl]phenyl]carbamate

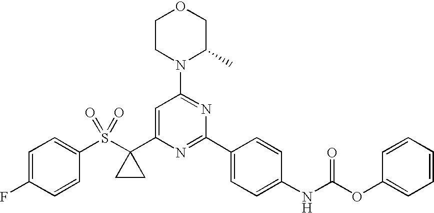

To a solution of 4-[4-[1-(4-fluorophenyl)sulfonylcyclopropyl]-6-[(3S)-3-methylmorpholin-4-yl]pyrimidin-2-yl]aniline (1.33 g, 2.84 mmol) in 1,4-dioxane (15 mL) was added sodium bicarbonate (0.358 g, 4.26 mmol) and phenyl chloroformate (0.357 mL, 2.84 mmol) and the reaction stirred at RT for 2 hours. The reaction mixture was diluted with DCM (20 mL), and washed with water (20 mL), the organic layer dried (MgSO₄), filtered and evaporated. The crude solid was triturated with diethyl ether to give a solid which was collected by filtration and dried under vacuum to give the desired product as a white solid (1.46 g).

NMR Spectrum: ¹H NMR (400.132 MHz, DMSO-d₆) δ 1.20 (3H, d), 1.60-1.65 (2H, m), 1.89-1.92 (2H, m), 3.18 (1H, dt), 3.47 (1H, dt), 3.61 (1H, dd), 3.75 (1H, d), 3.96 (1H, dd), 4.17 (1H, d), 4.45 (1H, s), 6.69 (1H, s), 7.25 (3H, d), 7.40-7.47 (4H, m), 7.55 (2H, d), 7.83-7.87 (2H, m), 7.92 (2H, d), 10.42 (1H, s)

LCMS Spectrum: m/z (ESI+) (M+H)+=589; HPLC tR=2.92 min.

### 4-[4-[1-(4-Fluorophenyl)sulfonylcyclopropyl]-6-[(3S)-3-methylmorpholin-4-yl]pyrimidin-2-yl]aniline

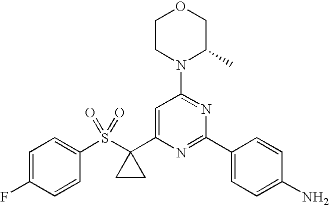

To a solution of 2-chloro-4-[1-(4-fluorophenyl)sulfonylcyclopropyl]-6-[(3S)-3-methylmorpholin-4-yl]pyrimidine (1.5 g, 3.64 mmol) in DMF (0.48 mL), DME (9.33 mL), water (4.0 mL) and ethanol (2.67 mL) was added 4-(4,4,5,5-tetramethyl-1,3,2-dioxaborolan-2-yl)aniline (1.037 g, 4.73 mmol), sodium carbonate (5 mL, 10.00 mmol) and dichlorobis(triphenylphosphine)palladium(II) (0.128 g, 0.18 mmol) and the suspension heated at 95° C. for 2 hours. The reaction mixture was cooled to RT, diluted with ethyl acetate (10 mL) and washed with water (2×10 mL). The organic layer was dried (MgSO₄), filtered and evaporated. The crude product was purified by flash silica chromatography, elution gradient 5 to 60% ethyl acetate in isohexane, to give the desired as a cream solid (1.33 g).

NMR Spectrum: ¹H NMR (400.132 MHz, DMSO-d₆) δ 1.17 (3H, d), 1.57-1.59 (2H, m), 1.86-1.88 (2H, m), 3.12 (1H, dt), 3.45 (1H, dt), 3.60 (1H, dd), 3.73 (1H, d), 3.95 (1H, dd), 4.10 (1H, d), 4.38 (1H, s), 5.52 (2H, s), 6.49 (2H, d), 6.55 (1H, s), 7.41 (2H, t), 7.64 (2H, d), 7.82-7.85 (2H, m)

LCMS Spectrum: m/z (ESI+) (M+H)+=469; HPLC tR=2.47 min.

### 2-Chloro-4-[1-(4-fluorophenyl)sulfonylcyclopropyl]-6-[(3S)-3-methylmorpholin-4-yl]pyrimidine

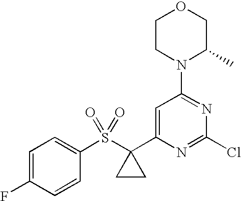

2-Chloro-4-[(4-fluorophenyl)sulfonylmethyl]-6-[(3S)-3-methylmorpholin-4-yl]pyrimidine (3.0 g, 7.78 mmol) was dissolved in DCM (40 mL) and sodium hydroxide concentrate (7.8 mL, 77.75 mmol) was added to the reaction, followed by dibromoethane (0.352 mL, 15.55 mmol). The reaction was stirred at 40° C. for 16 hours. The reaction mixture was washed with water (50 mL) and the organic layer dried (MgSO₄), filtered and evaporated to afford crude product.

The crude product was purified by flash silica chromatography, elution gradient 0 to 50% ethyl acetate in DCM, to give the desired material as a white solid (1.50 g,).

NMR Spectrum: ¹H NMR (400.132 MHz, DMSO-d₆) δ 1.15 (3H, d), 1.53-1.56 (2H, m), 1.82-1.85 (2H, m), 3.14 (1H, dt), 3.40 (1H, dt), 3.55 (1H, dd), 3.70 (1H, d), 3.91 (2H, dd), 4.25 (1H, s), 6.70 (1H, s), 7.45 (2H, t), 7.79-7.84 (2H, m)

LCMS Spectrum: m/z (ESI+)(M+H)+ 412, HPLC tR=2.14 min

### 2-Chloro-4-[(4-fluorophenyl)sulfonylmethyl]-6-[(3S)-3-methylmorpholin-4-yl]pyrimidine

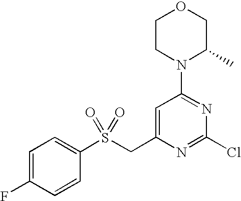

Triethylamine (1.117 ml, 8.01 mmol) was added to 2,4-dichloro-6-[(4-fluorophenyl)sulfonylmethyl]pyrimidine (2.34 g, 7.29 mmol) in DCM (36.4 mL) at 0° C. followed by (3S)-3-methylmorpholine (0.737 g, 7.29 mmol) in DCM (20 mL) over 15 minutes. The reaction was then stirred at RT for 16 hours. The reaction mixture was washed with water (50 mL), the organic layer dried (MgSO₄), filtered and evaporated to afford crude product. The crude product was purified by flash silica chromatography, elution gradient 0 to 50% ethyl acetate in DCM, to give the desired material as a beige solid (1.530 g).

NMR Spectrum: ¹H NMR (400.132 MHz, DMSO-d₆) δ 1.17 (3H, d), 3.13-3.20 (1H, m), 3.27-3.28 (1H, m), 3.39-3.46 (1H, m), 3.57 (1H, dd), 3.72 (1H, d), 3.93 (1H, dd), 4.17 (1H, s), 4.65 (2H, s), 6.71 (1H, s), 7.48 (2H, t), 7.83-7.87 (2H, m)

LCMS Spectrum: MH+ 386, retention time 1.94 min.

### 2,4-Dichloro-6-[(4-fluorophenyl)sulfonylmethyl]pyrimidine

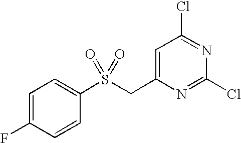

3-Chloroperoxybenzoic acid (3.78 g, 21.89 mmol) was added portionwise to 2,4-dichloro-6-[(4-fluorophenyl)sulfanylmethyl]pyrimidine (2.11 g, 7.30 mmol), in DCM (36.5 mL) and the reaction stirred at RT for 2 hours. The reaction mixture was washed with a saturated aqueous solution of sodium hydrogen carbonate (50 mL) and the organic layer dried (MgSO₄), filtered and evaporated to afford desired product (2.35 g).

NMR Spectrum: ¹H NMR (400.132 MHz, DMSO-d₆) δ 4.99 (2H, s), 7.48-7.52 (2H, m), 7.76 (1H, s), 7.85-7.88 (2H, m)

LCMS Spectrum: MH+ 319, retention time 2.01 min.

### 2,4-Dichloro-6-[(4-fluorophenyl)sulfanylmethyl]pyrimidine

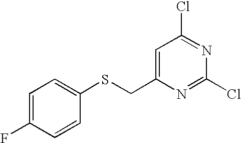

Phosphorus oxychloride (15.2 g, 99.1 mmol) was added to 6-[(4-fluorophenyl)sulfanylmethyl]-1H-pyrimidine-2,4-dione (2.5 g, 9.91 mmol), and the resulting 25 solution was stirred at reflux for 7 hours. The reaction was allowed to cool and the phosphorus oxychloride removed under reduced pressure to give a brown oil. This was dissolved in DCM and ice water (50 mL) added followed by solid sodium bicarbonate (until effervescence stops). The aqueous layer was extracted with DCM (2×50 mL) and the organics dried (MgSO₄), filtered and evaporated to afford crude product. The crude product was purified by flash silica chromatography, elution gradient 0 to 40% ethyl acetate in isohexane, to give the desired material as a yellow gum (2.11 g).

NMR Spectrum: ¹H NMR (400.132 MHz, DMSO-d₆) δ 4.21 (2H, s), 7.09-7.14 (2H, m), 7.34-7.38 (2H, m), 7.58 (1H, s)

LCMS Spectrum: M-H+ 287, retention time 2.51 min.

### 6-[(4-Fluorophenyl)sulfanylmethyl]-1H-pyrimidine-2,4-dione

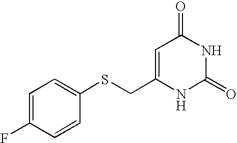

DBU (4.02 mL, 26.91 mmol) was added to 4-fluorobenzenethiol (3.45 g, 26.91 mmol), in DMF (90 mL) at RT. The resulting solution was stirred at 20° C. for 15 minutes. 6-(Chloromethyl)-1H-pyrimidine-2,4-dione (2.88 g, 17.94 mmol) was then added and the reaction stirred for 4 hours. The reaction mixture was concentrated and diluted with DCM (100 mL), and washed with water (100 mL). The aqueous layer was acidified with 2M hydrochloric acid to give a white solid which was filtered and washed with water then dried under vacuum to give desired product (2.5 g).

NMR Spectrum: ¹H NMR (400.132 MHz, DMSO-d₆) δ 3.80 (2H, s), 5.20 (1H, s), 7.18-7.23 (2H, m), 7.45-7.49 (2H, m), 10.90 (1H, s), 10.93 (1H, s)

LCMS Spectrum: M−H-251, retention time 0.80 min.

### EXAMPLE 9

### 1-[4-[4-(1-Cyclopentylsulfonylcyclopropyl)-6-[(3S)-3-methylmorpholin-4-yl]pyrimidin-2-yl]phenyl]-3-cyclopropyl-urea

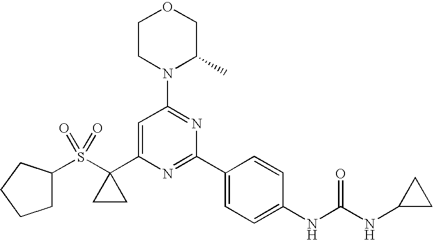

To a solution of phenyl N-[4-[4-(1-cyclopentylsulfonylcyclopropyl)-6-[(3S)-3-methylmorpholin-4-yl]pyrimidin-2-yl]phenyl]carbamate (200 mg, 0.36 mmol) and amine (1.44 mmol) in NMP (2 mL) was added triethylamine (0. 198 mL, 1.44 mmol) and mixture heated at 75° C. for 6 hours. The reaction was allowed to cool and purified by prep HPLC using decreasingly polar mixtures of water (containing 1% NH3) and acetonitrile as eluents, to give the desired material as a solid (130 mg).

NMR Spectrum: ¹H NMR (400.132 MHz, DMSO-d₆) δ 0.40-0.43 (2H, m), 0.62-0.67 (2H, m), 1.23 (3H, d), 1.50-1.70 (8H, m), 1.85-1.94 (2H, m), 1.99-2.07 (2H, m), 3.16-3.25 (1H, m), 3.49 (1H, dd), 3.63 (1H, dd), 3.76 (1H, d), 3.89-4.00 (2H, m), 4.20 (1H, d), 4.55 (1H, s), 6.46 (1H, s), 6.82 (1H, s), 7.51 (2H, d), 8.20 (2H, d), 8.54 (1H, s)

LCMS Spectrum: m/z (ESI+)(M+H)+=526; HPLC tR=2.42 min

mTOR Kinase Assay (Echo): 0.0018 μM

The compounds below were prepared in an analogous fashion from phenyl N-[4-[4-(1-cyclopentylsulfonylcyclopropyl)-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 9a | 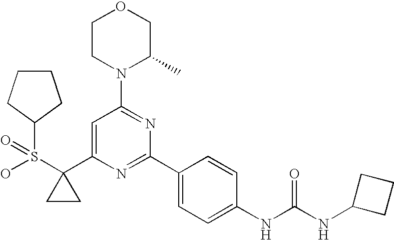 | 1-[4-[4-(1-cyclopentylsulfonylcyclopropyl)-6-[(3S)-3-methylmorpholin-4-yl]pyrimidin-2-yl]phenyl]-3-pyridin-2-yl-urea | 540 | 2.67 |
| 9b | 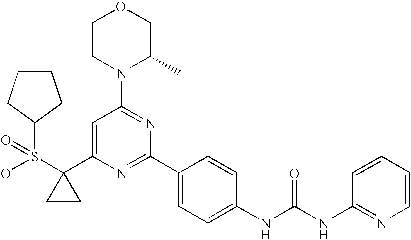 | 1-[4-[4-(1-cyclopentylsulfonylcyclopropyl)-6-[(3S)-3-methylmorpholin-4-yl]pyrimidin-2-yl]phenyl]-3-(2-methylpropyl)urea | 563 | 2.89 |
| 9c | 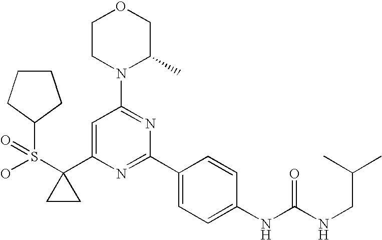 | 1-[4-[4-(1-cyclopentylsulfonylcyclopropyl)-6-[(3S)-3-methylmorpholin-4-yl]pyrimidin-2-yl]phenyl]-3-propan-2-yl-urea | 542 | 2.77 |
| 9d | 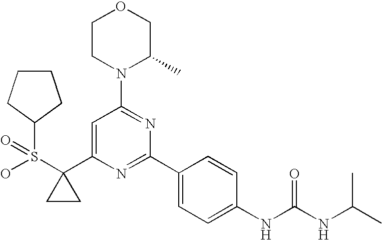 | 3-[4-[4-(1-cyclopentylsulfonylcyclopropyl)-6-[(3S)-3-methylmorpholin-4-yl]pyrimidin-2-yl]phenyl]-1-ethyl-urea | 528 | 2.59 |
| 9e | 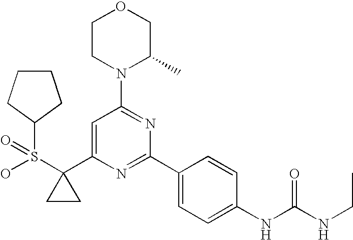 | 1-[4-[4-(1-cyclopentylsulfonylcyclopropyl)-6-[(3S)-3-methylmorpholin-4-yl]pyrimidin-2-yl]phenyl]-3-(2-dimethylaminoethyl)urea | 514 | 2.40 |
| 9f | 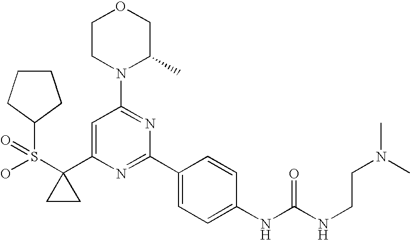 | 1-[4-[4-(1-cyclopentylsulfonylcyclopropyl)-6-[(3S)-3-methylmorpholin-4-yl]pyrimidin-2-yl]phenyl]-3-(2-hydroxyethyl)urea | 557 | 2.33 |
| 9g | 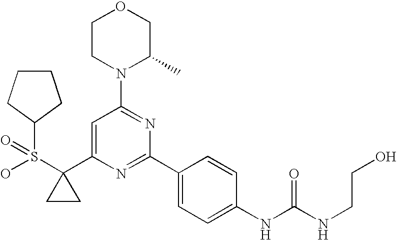 | 3-[4-[4-(1-cyclopentylsulfonylcyclopropyl)-6-[(3S)-3-methylmorpholin-4-yl]pyrimidin-2-yl]phenyl]-1-propyl-urea | 530 | 2.02 |
| 9h | 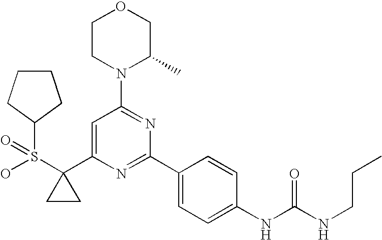 | 1-[4-[4-(1-cyclopentylsulfonylcyclopropyl)-6-[(3S)-3-methylmorpholin-4-yl]pyrimidin-2-yl]phenyl]-3-methyl-urea | 528 | 2.59 |
| 9i | 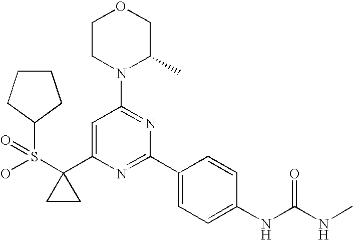 | 1-[4-[4-(1-cyclopentylsulfonylcyclopropyl)-6-[(3S)-3-methylmorpholin-4-yl]pyrimidin-2-yl]phenyl]-3-[4-(trifluoromethyl)phenyl]urea | 500 | 2.25 |
| 9j | 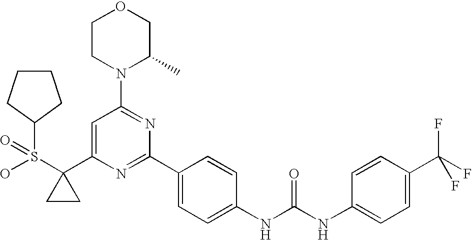 | 1-[4-[4-(1-cyclopentylsulfonylcyclopropyl)-6-[(3S)-3-methylmorpholin-4-yl]pyrimidin-2-yl]phenyl]-3-(1-hydroxy-2-methyl-propan-2-yl)urea | 630 | 3.25 |
| 9k | 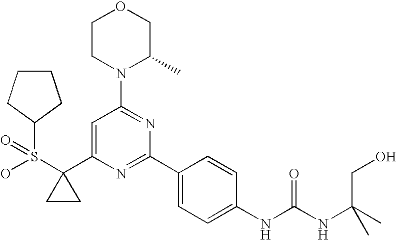 | 1-[4-[4-(1-cyclopentylsulfonylcyclopropyl)-6-[(3S)-3-methylmorpholin-4-yl]pyrimidin-2-yl]phenyl]-3-(3-hydroxypropyl)urea | 558 | 2.37 |
| 9l | 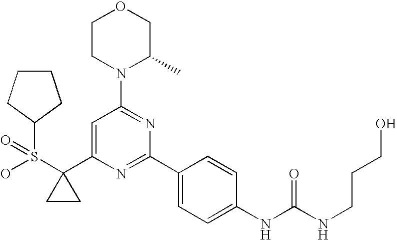 | 1-[4-[4-(1-cyclopentylsulfonylcyclopropyl)-6-[(3S)-3-methylmorpholin-4-yl]pyrimidin-2-yl]phenyl]-3-(1-methylpyrazol-4-yl)urea | 544 | 2.07 |
| 9m | 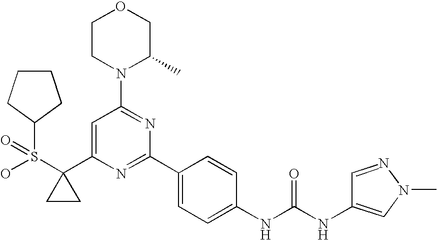 | 1-[4-[4-(1-cyclopentylsulfonylcyclopropyl)-6-[(3S)-3-methylmorpholin-4-yl]pyrimidin-2-yl]phenyl]-3-pyridin-2-yl-urea | 566 | 2.26 |

### EXAMPLE 9a

¹H NMR (400.132 MHz, DMSO-d₆) δ 1.23 (3H, d), 1.51-1.70 (1OH, m), 1.82-1.94 (4H, m), 1.98-2.08 (2H, m), 2.17-2.25 (2H, m), 3.17-3.25 (1H, m), 3.49 (1H, td), 3.63 (1H, dd), 3.76 (1H, d), 3.88-4.00 (2H, m), 4.10-4.22 (2H, m), 4.55 (1H, s), 6.47 (1H, d), 6.81 (1H, s), 7.48 (2H, d), 8.19 (2H, d), 8.56 (1H, s).

mTOR Kinase Assay (Echo): 0.0129 μM

### EXAMPLE 9b

¹H NMR (400.132 MHz, DMSO-d₆) δ 1.24 (3H, d), 1.53-1.70 (8H, m), 1.86-1.96 (2H, m), 2.00-2.09 (2H, m), 3.18-3.25 (1H, m), 3.46-3.54 (1H, m), 3.65 (1H, dd), 3.77 (1H, d), 3.90-4.01 (2H, m), 4.22 (1H, d), 4.57 (1H, s), 6.85 (1H, s), 7.04 (1H, t), 7.56 (1H, d), 7.65 (2H, d), 7.77 (1H, t), 8.26-8.32 (5H, m), 9.48 (1H, s), 10.63 (3H, s).

mTOR Kinase Assay (Echo): 0.0215 μM

### EXAMPLE 9c

¹H NMR (400.132 MHz, DMSO-d₆) δ 0.89 (6H, d), 1.23 (3H, d), 1.51-1.75 (8H, m), 1.85-1.96 (2H, m), 1.99-2.08 (2H, m), 2.94 (2H, t), 3.16-3.25 (1H, m), 3.49 (1H, td), 3.63 (1H, dd), 3.76 (1H, d), 3.89-4.00 (2H, m), 4.20 (1H, d), 4.55 (1H, s). 6.24 (1H, t), 6.82 (1H, s), 7.49 (2H, d), 8.19 (2H, d), 8.65 (1H, s).

mTOR Kinase Assay (Echo): 0.0483 μM

### EXAMPLE 9d

¹H NMR (400.132 MHz, DMSO-d₆) δ 1.11 (6H, d), 1.23 (3H, d), 1.51-1.69 (8H, m), 1.85-1.94 (2H, m), 1.98-2.08 (2H, m), 3.20 (1H, td), 3.49 (1H, td), 3.63 (1H, dd), 3.73-3.81 (2H, m), 3.89-4.00 (2H, m), 4.19 (1H, d), 4.55 (1H, s), 6.07 (1H, d), 6.83 (1H, s), 7.48 (2H, d), 8.19 (2H, d), 8.54 (1H, s).

mTOR Kinase Assay (Echo): 0.0151 μM

### EXAMPLE 9e

¹H NMR (400.132 MHz, DMSO-d₆) δ 1.07 (3H, t), 1.23 (3H, d), 1.49-1.71 (8H, m), 1.86-1.96 (2H, m), 1.99-2.07 (2H, m), 3.12 (2H, q), 3.17-3.25 (1H, m), 3.45-3.53 (1H, m), 3.61-3.66 (1H, m), 3.89-3.99 (2H, m), 4.19 (1H, d), 4.56 (1H, s), 6.17 (1H, t), 6.82 (1H, s), 7.50 (2H, d), 8.18 (2H, d), 8.67 (1H, s).

mTOR Kinase Assay (Echo): 0.00187 μM

### EXAMPLE 9f

¹H NMR (400.132 MHz, DMSO-d₆) δ 1.23 (3H, d), 1.50-1.70 (8H, m), 1.84-1.95 (2H, m), 1.98-2.08 (2H, m), 2.20 (6H, s), 2.33 (2H, t), 3.17-3.23 (2H, m), 3.49 (1H, td), 3.63 (1H, dd), 3.76 (1H, d), 3.90-4.00 (2H, m), 4.20 (1H, d), 4.56 (1H, s), 6.16 (1H, t), 6.82 (1H, s), 7.49 (2H, d), 8.19 (2H, d), 8.90 (1H, s).

mTOR Kinase Assay (Echo): 0. 144 μM

### EXAMPLE 9g

¹H NMR (400.132 MHz, DMSO-d₆) δ 1.23 (3H, d), 1.50-1.71 (8H, m), 1.85-1.95 (2H, m), 1.98-2.07 (2H, m), 3.14-3.24 (3H, m), 3.43-3.52 (3H, m), 3.63 (1H, d), 3.76 (1H, d), 3.89-4.00 (2H, m), 4.19 (1H, d), 4.55 (1H, s), 4.73 (1H, t), 6.26 (1H, t), 6.82 (1H, s), 7.49 (2H, d), 8.19 (2H, d), 8.81 (1H, s).

mTOR Kinase Assay (Echo): 0.00127 μM

### EXAMPLE 9h

¹H NMR (400.132 MHz, DMSO-d₆) δ 0.89 (3H, t), 1.23 (3H, d), 1.42-1.70 (10H, m), 1.84-1.94 (2H, m), 1.98-2.08 (2H, m), 3.03-3.09 (2H, m), 3.16-3.25 (1H, m), 3.50 (1H, d), 3.63 (1H, d), 3.76 (1H, d), 3.90-4.00 (2H, m), 4.20 (1H, d), 4.55 (1H, s), 6.21 (1H, t), 6.82 (1H, s), 7.49 (2H, d), 8.19 (2H, d), 8.66 (1H, s).

mTOR Kinase Assay (Echo): 0.0057 μM

### EXAMPLE 9i

¹H NMR (400.132 MHz, DMSO-d₆) δ 1.23 (3H, d), 1.49-1.71 (8H, m), 1.85-1.95 (2H, m), 1.99-2.08 (2H, m), 3.16-3.24 (1H, m), 3.49 (1H, td), 3.63 (1H, dd), 3.76 (1H, d), 3.87-4.01 (2H, m), 4.20 (1H, d), 4.56 (1H, s), 6.09 (1H, q), 6.82 (1H, s), 7.50 (2H, d), 8.19 (2H, d), 8.75 (1H, s).

mTOR Kinase Assay (Echo): 0.0024 μM

### EXAMPLE 9j

¹H NMR (400.132 MHz, DMSO-d₆) δ 1.24 (3H, d), 1.52-1.70 (8H, m), 1.87-1.96 (2H, m), 1.99-2.07 (2H, m), 3.17-3.27 (1H, m), 3.46-3.54 (1H, m), 3.64 (1H, d), 3.77 (1H, d), 3.89-4.01 (2H, m), 4.21 (1H, d), 4.57 (1H, s), 6.85 (1H, s), 7.59 (2H, d), 7.67 (4H, q), 8.27 (2H, d), 9.05 (1H, s), 9.15 (1H, s).

mTOR Kinase Assay (Echo): 0.046 μM

### EXAMPLE 9k

¹H NMR (400.132 MHz, DMSO-d₆) δ 1.21-1.26 (9H, m), 1.51-1.71 (8H, m), 1.86-1.93 (2H, m), 1.98-2.09 (2H, m), 3.16-3.24 (1H, m), 3.39 (2H, d), 3.49 (1H, dd), 3.63 (1H, dd), 3.76 (1H, d), 3.90-4.01 (2H, m), 4.20 (1H, d), 4.55 (1H, s), 4.95 (1H, t), 6.02 (1H, s), 6.81 (1H, s), 7.45 (2H, d), 8.18 (2H, d), 8.75 (1H, s).

mTOR Kinase Assay (Echo): 0.0115 μM

### EXAMPLE 9l

¹H NMR (400.132 MHz, DMSO-d₆) δ 1.23 (3H, d), 1.50-1.70 (8H, m), 1.85-1.95 (2H, m), 1.98-2.07 (2H, m), 3.12-3.25 (3H, m), 3.23-3.33 (2H, m), 3.43-3.53 (3H, m), 3.63 (1H, d), 3.77 (1H, d), 3.89-4.01 (2H, m), 4.20 (1H, d), 4.48 (1H, t), 4.56 (1H, s), 6.21 (1H, t), 6.81 (1H, s), 7.49 (2H, d), 8.19 (2H, d), 8.72 (1H, s).

mTOR Kinase Assay (Echo): 0.00395 μM

### EXAMPLE 9m

¹H NMR (400.132 MHz, DMSO-d₆) δ 1.23 (9H, d), 1.51-1.70 (20H, m), 1.86-1.95 (6H, m), 1.99-2.07 (4H, m), 3.15-3.25 (25H, m), 3.50 (1H, d), 3.64 (1H, d), 3.78 (1H, d), 3.90-4.01 (2H, m), 4.20 (1H, d), 4.56 (1H, s), 6.86 (1H, s), 7.43 (1H, s), 7.56 (2H, d), 7.81 (1H, s), 8.23 (2H, d), 8.44 (1H, s), 8.87 (1H, s).

mTOR Kinase Assay (Echo): 0.00401 μM

The preparation of phenyl N-[4-[4-(1-cyclopentylsulfonylcyclopropyl)-6-[(3S)-3-methylmorpholin-4-yl]pyrimidin-2-yl]phenyl]carbamate is described below.

### Phenyl N-[4-[4-(1-cyclopentylsulfonylcyclopropyl)-6-[(3S)-3-methylmorpholin-4-yl]pyrimidin-2-yl]phenyl]carbamate

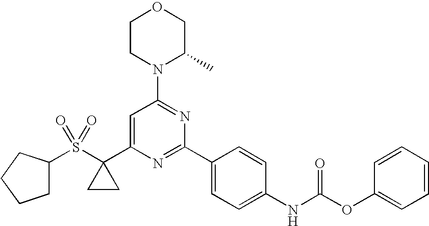

Phenyl chloroformate (1.701 mL, 13.56 mmol) was added to 4-[4-(1-cyclopentylsulfonylcyclopropyl)-6-[(3S)-3-methylmorpholin-4-yl]pyrimidin-2-yl]aniline (4 g, 9.04 mmol) and sodium hydrogen carbonate (1.139 g, 13.56 mmol) in dioxane (120 mL) cooled to 5° C. under nitrogen. The resulting mixture was stirred at RT for 2 hours. The reaction mixture was diluted with ethyl acetate (200 mL), and washed with water (125 mL). The organic layer was dried (MgSO₄), filtered and evaporated to afford crude material which was triturated with diethyl ether and isohexane to give the desired material as a white solid (4.77 g).

NMR Spectrum: ¹H NMR (400.132 MHz, DMSO-d₆) δ 1.24 (3H, d), 1.51-1.70 (4H, m), 1.85-1.95 (2H, m), 1.98-2.08 (2H, m), 3.21 (1H, td), 3.49 (1H, td), 3.64 (1H, dd), 3.77 (1H, d), 3.90-4.00 (2H, m), 4.21 (1H, d), 4.58 (1H, s), 6.88 (1H, s), 7.22-7.32 (3H, m), 7.41-7.49 (2H, m), 7.64 (2H, d), 8.30 (2H, d), 10.45 (1H, s)

LCMS Spectrum: m/z (ESI+)(M+H)+=563; HPLC tR=3.02 min

### 4-[4-(1-Cyclopentylsulfonylcyclopropyl)-6-[(3S)-3-methylmorpholin-4-yl]pyrimidin-2-yl]aniline

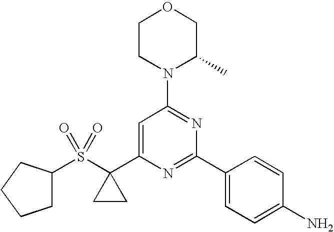

Bis(triphenylphosphine)palladium(II) chloride (300 mg, 0.43 mmol) was added to 2-chloro-4-(1-cyclopentylsulfonylcyclopropyl)-6-[(3S)-3-methylmorpholin-4-yl]pyrimidine (4.15 g, 10.75 mmol), 4-(4,4,5,5-tetramethyl-1,3,2-dioxaborolan-2-yl)aniline (3.53 g, 16.13 mmol) and sodium carbonate (25 mL, 50.0 mmol) in ethanol (20 mL), DMF (40 mL), water (25 mL) and DME (40 mL) at Rt and the resulting mixture degassed then stirred at 95° C. for 18 hours. The reaction mixture was diluted with ethyl acetate (400 mL), and washed with water (2×150 mL). The organic layer was dried (MgSO₄), filtered and evaporated to afford crude product. The crude product was purified by flash silica chromatography, elution gradient 10 to 80% ethyl acetate in isohexane, to give the desired material as a cream solid (4.00 g).

NMR Spectrum: ¹H NMR (400.132 MHz, DMSO-d₆) δ 1.21 (3H, d), 1.46-1.72 (7H, m), 1.84-1.95 (2H, m), 3.13-3.22 (1H, m), 3.47 (1H, td), 3.62 (1H, dd), 3.75 (1H, d), 3.89-3.99 (2H, m), 4.15 (1H, d), 4.54 (1H, s), 5.58 (2H, s), 6.61 (2H, d), 6.72 (1H, s), 8.02 (2H, d)

LCMS Spectrum: m/z (ESI+)(M+H)+=443; HPLC tR=2.4 min

### 2-Chloro-4-(1-cyclopentylsulfonylcyclopropyl)-6-[(3S)-3-methylmorpholin-4-yl]pyrimidine

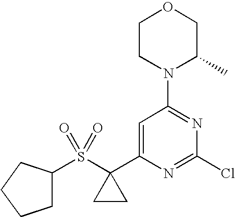

Sodium hydroxide (62.5 mL, 125.04 mmol) was added to 2-chloro-4-(cyclopentylsulfonylmethyl)-6-[(3S)-3-methylmorpholin-4-yl]pyrimidine (4.50 g, 12.50 mmol), 1,2-dibromoethane (4.31 mL, 50.02 mmol) and tetrabutylammonium bromide (0.403 g, 1.25 mmol) in toluene (100 mL) at 30° C. under nitrogen. The resulting mixture was stirred at 60° C. for 3 hours. The reaction mixture was diluted with ethyl acetate (200 mL), and washed with water (100 mL). The organic layer was dried (MgSO₄), filtered and evaporated to afford crude product. The crude product was purified by flash silica chromatography, elution gradient 5 to 45% ethyl acetate in isohexane, to give the desired material as a colourless gum (4.47 g).

NMR Spectrum: ¹H NMR (400.132 MHz, DMSO-d₆) δ 1.21 (3H, d), 1.47-1.68 (8H, m), 1.80-1.89 (2H, m), 1.91-1.98 (2H, m), 3.21 (1H, dt), 3.44 (1H, td), 3.58 (1H, dd), 3.72 (1H, d), 3.82 (1H, q), 3.93 (1H, dd), 3.98-4.06 (1H, m), 4.41 (1H, s), 6.97 (1H, s)

### 2-Chloro-4-(cyclopentylsulfonylmethyl)-6-[(3S)-3-methylmorpholin-4-yl]pyrimidine

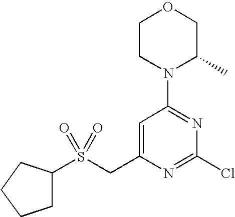

Hydrogen peroxide (19.54 mL, 632 mmol) was added to 2-chloro-4-(cyclopentylsulfanylmethyl)-6-[(3S)-3-methylmorpholin-4-yl]pyrimidine (10.36 g, 31.60 mmol), sodium tungstate dihydrate (0.208 g, 0.63 mmol) (dissolved in minimum quantity of water) and 2M sulphuric acid solution (0.177 mL) in dioxane (100 mL) at 55° C. under air. The resulting solution was stirred at 55° C. for 2 hours. The reaction mixture was diluted with ethyl acetate (100 mL), and washed with water then a 10% aqueous solution of sodium metabisulfite. The organic layer was dried (MgSO₄), filtered and evaporated to afford crude product. The crude product was purified by flash silica chromatography, elution gradient 5 to 70% ethyl acetate in isohexane, to give the desired material as a colourless gum (9.7 g).

NMR Spectrum: ¹H NMR (400.132 MHz, DMSO-d₆) δ 1.22 (3H, d), 1.64 (4H, m), 1.95 (4H, m), 3.24 (1H, m), 3.45 (1H, td), 3.60 (1H, dd), 3.71 (1H, m), 3.95 (2H, m), 4.35 (1H, s), 4.40 (2H, s), 6.91 (1H, s)

Mass Spectrum: m/z (ESI+)(M+H)+=360

### 2-Chloro-4-(cyclopentylsulfanylmethyl)-6-[(3S)-3-methylmorpholin-4-yl]pyrimidine

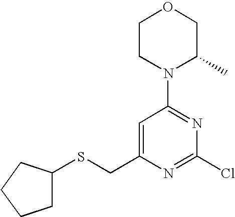

DIPEA (9.62 mL, 55.57 mmol) was added to cyclopentanethiol (5.93 mL, 55.57 mmol), in DMF (80 mL) at RT under nitrogen. The resulting solution was stirred at RT for 20 minutes. 2-Chloro-4-(iodomethyl)-6-[(3S)-3-methylmorpholin-4-yl]pyrimidine (13.1 g, 37.05 mmol) was added to the reaction and stirred for 2 hours at RT. The reaction mixture was diluted with ethyl acetate (500 mL), and washed with water (2×200 mL). The organic layer was dried (MgSO₄), filtered and evaporated to afford crude product. The crude product was purified by flash silica chromatography, elution gradient 0 to 30% ethyl acetate in isohexane, to give the desired material as a colourless gum (11.13 g).

NMR Spectrum: ¹H NMR (400.132 MHz, DMSO-d₆) δ 1.20 (3H, d), 1.43 (2H, m), 1.53 (2H, m), 1.65 (2H, m), 1.94 (2H, m), 3.16 (2H, m), 3.44 (1H, td), 3.71 (1H, d), 3.95 (2H, m), 4.35 (1H, s), 6.79 (1H, s)

Mass Spectrum: m/z (ESI+)(M+H)+=328

The preparation of 2-chloro-4-(iodomethyl)-6-[(3S)-3-methylmorpholin-4-yl]pyrimidine was described earlier

### EXAMPLE 10

### 3-Cyclopropyl-1-[4-[4-[(3S)-3-methylmorpholin-4-yl]-6-[1-[2-(trifluoromethyl)phenyl]sulfonylcyclopropyl]pyrimidin-2-yl]phenyl]urea

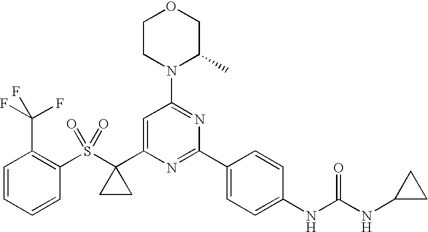

To a solution of phenyl N-[4-[4-[(3S)-3-methylmorpholin-4-yl]-6-[1-[2-(trifluoromethyl)phenyl]sulfonylcyclopropyl]pyrimidin-2-yl]phenyl]carbamate (200 mg,0.31 mmol) and cyclopropylamine (1.25 mmol) in NMP (2 mL) was added triethylamine (0.175 mL, 1.25 mmol). The reaction mixture was heated at 75° C. for 6 hours. The reaction mixture was purified by prep HPLC, using decreasingly polar mixtures of water (containing 1% NH3) and acetonitrile as eluents, to give the desired material (140 mg).

NMR Spectrum: ¹H NMR (400.132 MHz, DMSO-d₆) δ 0.39-0.44 (2H, m), 0.63-0.67 (2H, m), 1.14 (3H, d), 1.70-1.75 (2H, m), 1.92-1.95 (2H, m), 3.11 (1H, td), 3.39-3.48 (1H, m), 3.59 (1H, dd), 3.73 (1H, d), 3.94 (1H, dd), 4.09 (1H, d), 4.43 (1H, s), 6.41 (1H, d), 6.62 (1H, d), 6.62 (1H, s), 7.37 (2H, d), 7.76 (2H, d), 7.82-7.86 (2H, m), 7.91-7.95 (1H, m), 8.16-8.19 (1H, m), 8.49 (1H, s)

LCMS Spectrum: m/z (ESI+)(M+H)+=602; HPLC tR=2.52 min.

mTOR Kinase Assay (Echo): 0.00448 μM

The following compounds were prepared in an analogous fashion from phenyl N-[4-[4-[(3S)-3-methylmorpholin-4-yl]-6-[1-[2-(trifluoromethyl)phenyl]sulfonylcyclopropyl]pyrimidin-2-yl]phenyl]carbamate using the appropriate amine.

| Ex- | | | | |
|---|---|---|---|---|
| am- | | | LCMS | Retention |
| ple | Structure | NAME | MH+ | time (min) |
| 10a | 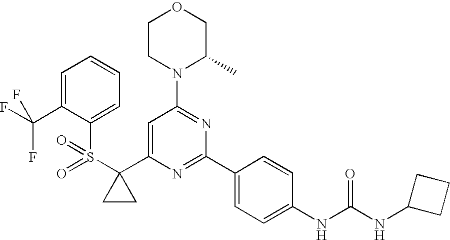 | 3-cyclobutyl-1-[4-[4-[(3S)-3-methylmorpholin-4-yl]-6-[1-[2-(trifluoromethyl)phenyl]sulfonylcyclopropyl]pyrimidin-2-yl]phenyl]urea | 616 | 2.73 |
| 10b | 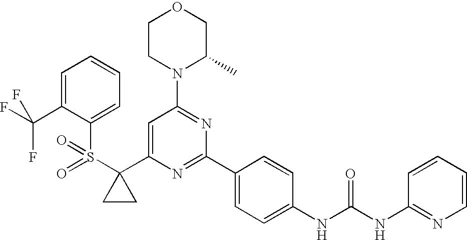 | 1-[4-[4-[(3S)-3-methylmorpholin-4-yl]-6-[1-[2-(trifluoromethyl)phenyl]sulfonylcyclopropyl]pyrimidin-2-yl]phenyl]-3-pyridin-2-yl-urea | | |
| 10c | 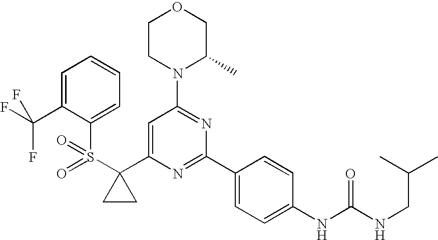 | 1-[4-[4-[(3S)-3-methylmorpholin-4-yl]-6-[1-[2-(trifluoromethyl)phenyl]sulfonylcyclopropyl]pyrimidin-2-yl]phenyl]-3-(2-methylpropyl)urea | 618 | 2.81 |
| 10d | 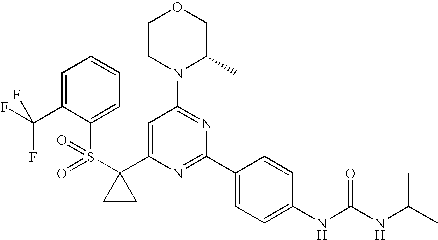 | 1-[4-[4-[(3S)-3-methylmorpholin-4-yl]-6-[1-[2-(trifluoromethyl)phenyl]sulfonylcyclopropyl]pyrimidin-2-yl]phenyl]-3-propan-2-yl-urea | 604 | 2.66 |
| 10e | 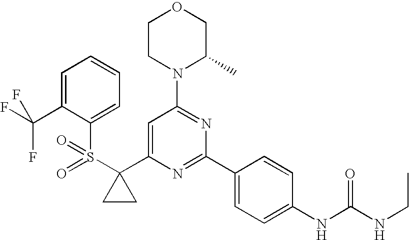 | 1-ethyl-3-[4-[4-[(3S)-3-methylmorpholin-4-yl]-6-[1-[2-(trifluoromethyl)phenyl]sulfonylcyclopropyl]pyrimidin-2-yl]phenyl]urea | 590 | 2.51 |
| 10f | 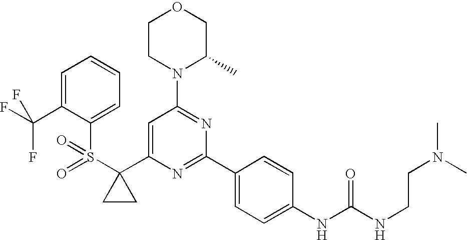 | 3-(2-dimethylaminomethyl)-1-[4-[4-[(3S)-3-methylmorpholin-4-yl]-6-[1-[2-(trifluoromethyl)phenyl]sulfonylcyclopropyl]pyrimidin-2-yl]phenyl]urea | 633 | 2.46 |
| 10g | 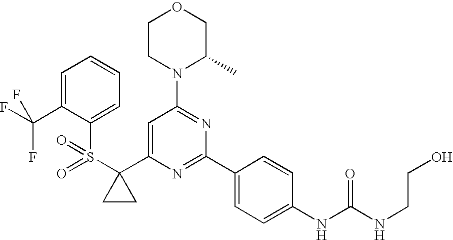 | 3-(2-hydroxymethyl)-1-[4-[4-[(3S)-3-methylmorpholin-4-yl]-6-[1-[2-(trifluoromethyl)phenyl]sulfonylcyclopropyl]pyrimidin-2-yl]phenyl]urea | 606 | 2.16 |
| 10h | 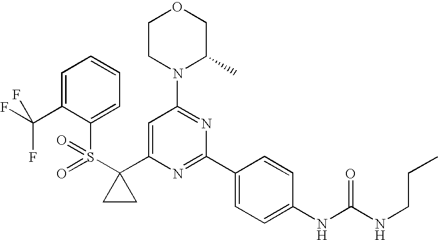 | 3-[4-[4-[(3S)-3-methylmorpholin-4-yl]-6-[1-[2-(trifluoromethyl)phenyl]sulfonylcyclopropyl]pyrimidin-2-yl]phenyl]-1-propyl-urea | 604 | 2.62 |
| 10i | 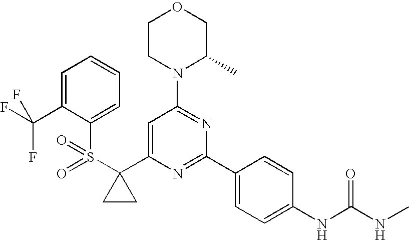 | 3-methyl-1-[4-[4-[(3S)-3-methylmorpholin-4-yl]-6-[1-[2-(trifluoromethyl)phenyl]sulfonylcyclopropyl]pyrimidin-2-yl]phenyl]urea | 576 | 2.34 |
| 10j | 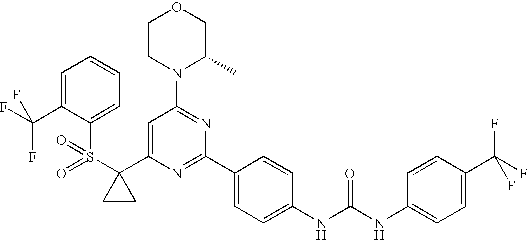 | 1-[4-[4-[(3S)-3-methylmorpholin-4-yl]-6-[1-[2-(trifluoromethyl)phenyl]sulfonylcyclopropyl]pyrimidin-2-yl]phenyl]-3-[4-(trifluoromethyl)phenyl]urea | 706 | 3.22 |
| 10k | 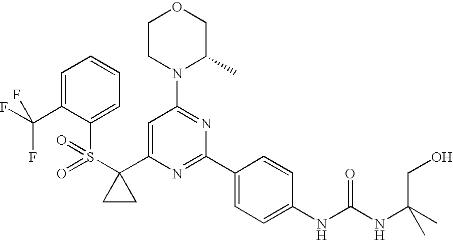 | 3-(1-hydroxy-2-methyl-propan-2-yl)-1-[4-[4-[(3S)-3-methylmorpholin-4-yl]-6-[1-[2-(trifluoromethyl)phenyl]sulfonylcyclopropyl]pyrimidin-2-yl]phenyl]urea | 634 | 2.43 |
| 10l | 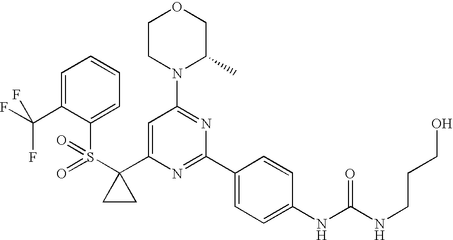 | 3-(3-hydroxypropyl)-1-[4-[4-[(3S)-3-methylmorpholin-4-yl]-6-[1-[2-(trifluoromethyl)phenyl]sulfonylcyclopropyl]pyrimidin-2-yl]phenyl]urea | 620 | 2.17 |
| 10m | 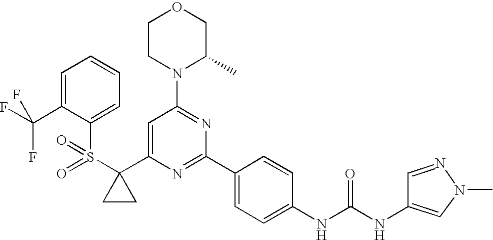 | 1-[4-[4-[(3S)-3-methylmorpholin-4-yl]-6-[1-[2-(trifluoromethyl)phenyl]sulfonylcyclopropyl]pyrimidin-2-yl]phenyl]-3-(1-methylpyrazol-4-yl)urea | 642 | 2.35 |

### EXAMPLE 10a

¹H NMR (400.132 MHz, DMSO-d₆) δ 1.13 (3H, d), 1.56-1.75 (4H, m), 1.80-1.96 (4H, m), 2.17-2.25 (2H, m), 3.11 (1H, td), 3.39-3.48 (1H, m), 3.59 (1H, dd), 3.73 (1H, d), 3.94 (1H, dd), 4.04-4.18 (2H, m), 4.43 (1H, s), 6.44 (1H, d), 6.61 (1H, s), 7.34 (2H, d), 7.75 (2H, d), 7.82-7.86 (2H, m), 7.92-7.95 (1H, m), 8.15-8.20 (1H, m), 8.52 (1H, s).

mTOR Kinase Assay (Echo): 0.0395 μM

### EXAMPLE 10b

mTOR Kinase Assay (Echo): 0.0266 μM

### EXAMPLE 10c

¹H NMR(400.132 MHz, DMSO-d₆) δ 0.88 (6H, d), 1.14 (3H, d), 1.67-1.75 (2H, m), 1.91-1.95 (2H, m), 2.94 (2H, t), 3.11 (1H, td), 3.44 (1H, td), 3.59 (1H, dd), 3.73 (1H, d), 3.94 (1H, dd), 4.09 (1H, d), 4.43 (1H, s), 6.22 (1H, t), 6.61 (1H, s), 7.35 (2H, d), 7.76 (2H, d), 7.81-7.87 (2H, m), 7.92-7.96 (1H, m), 8.16-8.21 (1H, m), 8.60 (1H, s).

mTOR Kinase Assay (Echo): 0.147 μM

### EXAMPLE 10d

¹H NMR (400.132 MHz, DMSO-d₆) δ 1.09-1.15 (9H, m), 1.68-1.77 (2H, m), 1.92-1.95 (2H, m), 3.11 (1H, td), 3.44 (1H, td), 3.59 (1H, dd), 3.71-3.82 (2H, m), 3.94 (1H, dd), 4.09 (1H, d), 4.42 (1H, s), 6.04 (1H, d), 6.61 (1H, s), 7.34 (2H, d), 7.75 (2H, d), 7.82-7.87 (2H, m), 7.91-7.95 (1H, m), 8.16-8.20 (1H, m), 8.49 (1H, s).

mTOR Kinase Assay (Echo): 0.0432 μM

### EXAMPLE 10e

¹H NMR (400.132 MHz, DMSO-d₆) δ 1.06 (3H, t), 1.13 (3H, d), 1.69-1.75 (2H, m), 1.91-1.95 (2H, m), 3.07-3.19 (3H, m), 3.44 (1H, td), 3.59 (1H, dd), 3.73 (1H, d), 3.94 (1H, dd), 4.04-4.12 (1H, m), 4.44 (1H, s), 6.14 (1H, t), 6.62 (1H, s), 7.36 (2H, d), 7.75 (2H, d), 7.81-7.85 (2H, m), 7.91-7.95 (1H, m), 8.16-8.20 (1H, m), 8.62 (1H, s).

mTOR Kinase Assay (Echo): 0.00691 μM

### EXAMPLE 10f

¹H NMR (400.132 MHz, DMSO-d₆) δ 1.14 (3H, d), 1.69-1.74 (2H, m), 1.91-1.95 (2H, m), 2.19 (6H, s), 2.33 (2H, t), 3.11 (1H, td), 3.16-3.22 (2H, m), 3.44 (1H, td), 3.59 (1H, dd), 3.73 (1H, d), 3.94 (1H, dd), 4.09 (1H, d), 4.43 (1H, s), 6.15 (1H, t), 6.62 (1H, s), 7.35 (2H, d), 7.75 (2H, d), 7.82-7.87 (2H, m), 7.91-7.96 (1H, m), 8.16-8.20 (1H, m), 8.86 (1H, s).

mTOR Kinase Assay (Echo): 0.174 μM

### EXAMPLE 10g

¹H NMR (400.132 MHz, DMSO-d₆) δ 1.13 (3H, d), 1.68-1.76 (2H, m), 1.92-1.95 (2H, m), 3.08-3.20 (3H, m), 3.39-3.48 (3H, m), 3.59 (1H, dd), 3.74 (1H, d), 3.94 (1H, dd), 4.09 (1H, d), 4.43 (1H, s), 4.72 (1H, t), 6.23 (1H, t), 6.62 (1H, s), 7.35 (2H, d), 7.75 (2H, d), 7.82-7.85 (2H, m), 7.92-7.95 (1H, m), 8.16-8.19 (1H, m), 8.76 (1H, s).

mTOR Kinase Assay (Echo): 0.00156 μM

### EXAMPLE 10h

¹H NMR (400.132 MHz, DMSO-d₆) δ 0.88 (3H, t), 1.14 (3H, d), 1.45 (2H, q), 1.70-1.74 (2H, m), 1.90-1.95 (2H, m), 3.03-3.15 (3H, m), 3.40-3.48 (1H, m), 3.59 (1H, dd), 3.73 (1H, d), 3.94 (1H, dd), 4.09 (1H, d), 4.41 (1H, s), 6.18 (1H, t), 6.62 (1H, s), 7.36 (2H, d), 7.75 (2H, d), 7.82-7.87 (2H, m), 7.92-7.95 (1H, m), 8.17-8.20 (1H, m), 8.61 (1H, s).

mTOR Kinase Assay (Echo): 0.0268 μM

### EXAMPLE 10i

¹H NMR (400.132 MHz, DMSO-d₆) δ 1.13 (3H, d), 1.69-1.75 (2H, m), 1.92-1.95 (2H, m), 2.66 (3H, d), 3.11 (1H, td), 3.44 (1H, td), 3.59 (1H, dd), 3.73 (1H, d), 3.94 (1H, dd), 4.09 (1H, d), 4.43 (1H, s), 6.04 (1H, q), 6.61 (1H, s), 7.37 (2H, d), 7.76 (2H, d), 7.82-7.85 (2H, m), 7.92-7.95 (1H, m), 8.16-8.19 (1H, m), 8.70 (1H, s).

mTOR Kinase Assay (Echo): 0.00591 μM

### EXAMPLE 10j

¹H NMR (400.132 MHz, DMSO-d₆) δ 1.14 (3H, d), 1.71-1.76 (2H, m), 1.91-1.96 (2H, m), 3.09-3.17 (1H, m), 3.45 (1H, dd), 3.60 (1H, dd), 3.74 (1H, d), 3.95 (1H, d), 4.11 (1H, d), 4.44 (1H, s), 6.64 (1H, s), 7.45 (2H, d), 7.63-7.70 (4H, m), 7.81-7.87 (4H, m), 7.93-7.96 (1H, m), 8.17-8.21 (1H, m), 9.01 (1H, s), 9.12 (1H, s).

mTOR Kinase Assay (Echo): 0.11μM

### EXAMPLE 10k

¹H NMR (400.132 MHz, DMSO-d₆) δ 1.14 (3H, d), 1.25 (6H, s), 1.69-1.75 (2H, m), 1.91-1.95 (2H, m), 3.11 (1H, td), 3.37-3.48 (3H, m), 3.59 (1H, dd), 3.74 (1H, d), 3.94 (1H, dd), 4.09 (1H, d), 4.42 (1H, s), 4.95 (1H, t), 5.99 (1H, s), 6.62 (1H, s), 7.32 (2H, d), 7.74 (2H, d), 7.82-7.87 (2H, m), 7.92-7.95 (1H, m), 8.17-8.20 (1H, m), 8.69 (1H, s).

mTOR Kinase Assay (Echo): 0.0502 μM

### EXAMPLE 10l

¹H NMR (400.132 MHz, DMSO-d₆) δ 1.14 (3H, d), 1.56-1.63 (2H, m), 1.69-1.74 (2H, m), 1.91-1.95 (2H, m), 3.07-3.19 (3H, m), 3.40-3.50 (3H, m), 3.59 (1H, dd), 3.73 (1H, d), 3.94 (1H, dd), 4.09 (1H, d), 4.43 (1H, s), 4.47 (1H, t), 6.18 (1H, t), 6.62 (1H, s), 7.36 (2H, d), 7.76 (2H, d), 7.82-7.86 (2H, m), 7.91-7.96 (1H, m), 8.15-8.20 (1H, m), 8.67 (1H, s).

mTOR Kinase Assay (Echo): 0.0183 μM

### EXAMPLE 10m

¹H NMR (400.132 MHz, DMSO-d₆) δ 1.14 (3H, d), 1.70-1.75 (2H, m), 1.90-1.96 (2H, m), 3.12 (1H, td), 3.44 (1H, td), 3.59 (1H, dd), 3.73 (1H, d), 3.95 (1H, dd), 4.10 (1H, d), 4.43 (1H, s), 6.62 (1H, s), 7.37-7.44 (3H, m), 7.76-7.81 (3H, m), 7.83-7.87 (2H, m), 7.92-7.95 (1H, m), 8.17-8.20 (1H, m), 8.38 (1H, s), 8.80 (1H, s).

mTOR Kinase Assay (Echo): 0.00497 μM

The preparation of phenyl N-[4-[4-[(3S)-3-methylmorpholin-4-yl]-6-[1-[2-(trifluoromethyl)phenyl]sulfonylcyclopropyl]pyrimidin-2-yl]phenyl]carbamate is described below.

### Phenyl N-[4-[4-[(3S)-3-methylmorpholin-4-yl]-6-[1-[2-(trifluoromethyl)phenyl]sulfonylcyclopropyl]pyrimidin-2-yl]phenyl]carbamate

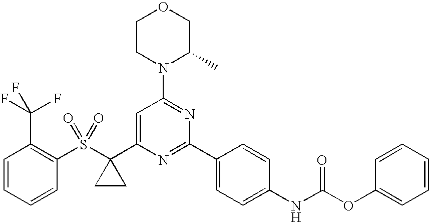

Phenyl chloroformate (1.669 mL, 13.31 mmol) was added to 4-[4-[(3S)-3-methylmorpholin-4-yl]-6-[1-[2-(trifluoromethyl)phenyl]sulfonylcyclopropyl]pyrimidin-2-yl]aniline (4.6 g, 8.87 mmol) and sodium hydrogen carbonate (1.118 g, 13.31 mmol) in dioxane (20 mL) at 5° C. under nitrogen. The resulting mixture was stirred at RT for 2 hours then the reaction mixture diluted with ethyl acetate (200 mL), and washed with water (125 mL). The organic layer was dried (MgSO₄), filtered and evaporated to afford crude product which was triturated with a mixture of diethyl ether and isohexane to give the desired material (4.55 g).

NMR Spectrum: ¹H NMR (400.132 MHz, DMSO-d₆) δ 1.14 (3H, d), 1.71-1.76 (2H, m), 1.92-1.96 (2H, m), 3.12 (1H, td), 3.41-3.47 (1H, m), 3.59 (1H, d), 3.73 (1H, d), 3.95 (1H, dd), 4.11 (1H, d), 4.45 (1H, s), 6.65 (1H, s), 7.22-7.31 (2H, m), 7.43-7.52 (3H,

### 4-[4-[(3S)-3-Methylmorpholin-4-yl]-6-[1-[2-(trifluoromethyl)phenyl]sulfonylcyclopropyl]pyrimidin-2-yl]aniline

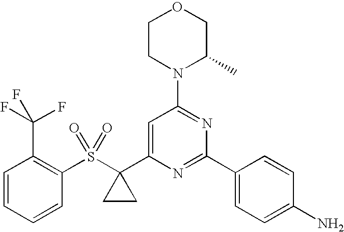

Bis(triphenylphosphine)palladium(II) chloride (300 mg, 0.43 mmol) was added to 2-chloro-4-[(3S)-3-methylmorpholin-4-yl]-6-[1-[2-(trifluoromethyl)phenyl]sulfonylcyclopropyl]pyrimidine (5 g, 10.83 mmol), 4-(4,4,5,5-tetramethyl-1,3,2-dioxaborolan-2-yl)aniline (3.56 g, 16.24 mmol) and sodium carbonate (20 mL, 40.0 mmol) in a mixture of ethanol (10 mL), DMF (20 mL), water (15 mL) and DME (40 mL) at RT. The resulting mixture was degassed then stirred at 95° C. for 18 hours. The reaction was allowed to cool, diluted with ethyl acetate (400 mL), and washed with water (2×200 mL). The combined organics were dried (MgSO₄), filtered and evaporated. The crude product was purified by flash silica chromatography, elution gradient 10 to 70% ethyl acetate in isohexane, to give the desired material as a cream solid (5.40 g).

NMR Spectrum: ¹H NMR (400.132 MHz, DMSO-d₆) δ 1.11 (3H, d), 1.68-1.73 (2H, m), 1.90-1.93 (2H, m), 3.07 (1H, td), 3.38-3.47 (1H, m), 3.58 (1H, dd), 3.72 (1H, d), 3.93 (2H, dd), 4.02-4.07 (1H, m), 4.39 (1H, s), 5.50 (2H, s), 6.45-6.50 (3H, m), 7.61 (2H, d), 7.81-7.85 (2H, m), 7.93 (1H, dd), 8.17 (1H, dd)

LCMS Spectrum: m/z (ESI+)(M+H)+=519; HPLC tR=2.51 min.

### 2-Chloro-4-[(3S)-3-methylmorpholin-4-yl]-6-[1-[2-(trifluoromethyl)phenyl]sulfonylcyclopropyl]pyrimidine

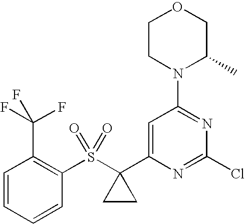

An aqueous solution of sodium hydroxide (30 mL, 247.8 mmol) was added to 2-chloro-4-[(3S)-3-methylmorpholin-4-yl]-6-[[2-(trifluoromethyl)phenyl]sulfonylmethyl]pyrimidine (4.5 g, 10.32 mmol), 1,2-dibromoethane (4.45 mL, 51.62 mmol) and tetrabutylammonium bromide (0.333 g, 1.03 mmol) in toluene (100 mL) at 30° C. under nitrogen. The resulting mixture was stirred at 30° C. for 3 hours. The reaction mixture was diluted with ethyl acetate (200 mL), and washed with water (100 mL). The organic layer was dried (MgSO₄), filtered and evaporated to afford crude product. The crude product was purified by flash silica chromatography, elution gradient 5 to 60% ethyl acetate in isohexane, to give the desired material (4.70 g).

NMR Spectrum: ¹H NMR (400.132 MHz, DMSO-d₆) δ 1.10 (3H, d), 1.61-1.66 (2H, m), 1.86-1.91 (2H, m), 3.09 (1H, td), 3.37 (1H, td), 3.52 (1H, dd), 3.68 (1H, d), 3.84-3.93 (2H, m), 4.27 (1H, s), 6.69 (1H, s), 7.85-7.93 (2H, m), 8.00 (1H, d), 8.07 (1H, d)

LCMS Spectrum: m/z (ESI+)(M+H)+=462; HPLC tR=2.43 min.

### 2-Chloro-4-[(3S)-3-methylmorpholin-4-yl]-6-[[2-(trifluoromethyl)phenyl]sulfonylmethyl]pyrimidine

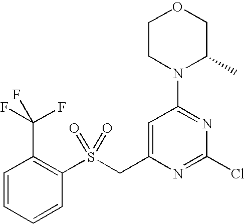

Sodium 2-(trifluoromethyl)benzenesulfinate (10.24 g, 44 mmol) was added to 2-chloro-4-(iodomethyl)-6-[(3S)-3-methylmorpholin-4-yl]pyrimidine (13 g, 36.77 mmol), in acetonitrile (500 mL) at RT under nitrogen. The resulting mixture was stirred at 80° C. for 3 hours. Additional sodium 2-(trifluoromethyl)benzenesulfinate (10.2 g, 44 mmol) was added and reaction heated at 80° C. for 1 hour. The reaction mixture allowed to cool and concentrated in vacuo. The material was dissolved in ethyl acetate (500 ML), and washed with water (200 mL). The organic layer was dried (MgSO₄), filtered and evaporated to afford crude product. The crude product was purified by flash silica chromatography, elution gradient 0 to 40% ethyl acetate in isohexane, to give the desired material as an orange/cream solid (9.48 g).

NMR Spectrum: ¹H NMR (400.132 MHz, DMSO-d₆) δ 1.18 (3H, d), 3.17 (1H, td), 3.43 (1H, td), 3.58 (1H, dd), 3.72 (1H, d), 3.93 (2H, m), 4.27 (1H, s), 4.68 (2H, s), 6.79 (1H, s), 7.94 (3H, m), 8.08 (1H, d)

LCMS Spectrum: m/z (ESI+)(M+H)+=436; HPLC tR=2.35 min.

### Sodium 2-(trifluoromethyl)benzenesulfinate

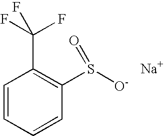

Sodium sulfate (3.92 mL, 81.88 mmol) was dissolved in water and stirred at RT 10 minutes. Sodium bicarbonate (13.74 g, 163.52 mmol) was added and the mixture stirred at 50° C. for 1 hour. 2-(Trifluoromethyl)benzene-1-sulfonyl chloride (12.62 mL, 81.76 mmol) was added dropwise to the reaction mixture which was then stirred at 50° C. for 18 hours. The reaction mixture was evaporated to dryness and the residue suspended in methanol (250 mL) and stirred at RT for 20 minutes. The solid was removed by filtration and the filtrate evaporated to give the desired material (20.00 g).

NMR Spectrum: ¹H NMR (400.132 MHz, DMSO-d₆) δ 7.40 (1H, d), 7.51 (1H, d), 7.64 (1H, d), 8.05 (1H, d)

The preparation of 2-chloro-4-(iodomethyl)-6-[(3S)-3-methylmorpholin-4-yl]pyrimidine was described earlier.

### EXAMPLE 11

### 3-Cyclopropyl-1-[4-[4-(1-ethylsulfonylcyclopropyl)-6-[(3S)-3-methylmorpholin-4-yl]pyrimidin-2-yl]phenyl]urea

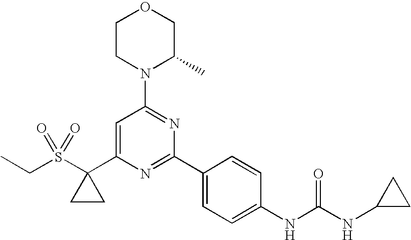

To a solution of phenyl N-[4-[4-(1-ethylsulfonylcyclopropyl)-6-[(3S)-3-methylmorpholin-4-yl]pyrimidin-2-yl]phenyl]carbamate (150 mg, 0.29 mmol) in DMF (2 mL) was added triethylamine (0. 120 mL, 0.86 mmol) followed by cyclopropylamine (0. 100 mL, 1.44 mmol) and the reaction heated at 50° C. for 2 hours. The crude product was purified by preparative HPLC using decreasingly polar mixtures of water (containing 1% NH3) and acetonitrile as eluents, to give the desired material as a white solid (90 mg).

NMR Spectrum: ¹H NMR (400.13 MHz, DMSO-d₆) δ 0.40-0.44 (2H, m), 0.63-0.67 (2H, m), 1.23 (3H, d), 1.32 (3H, t), 1.55 (2H, t), 1.62-1.65 (2H, m), 2.52-2.58 (1H, m), 3.17-3.24 (1H, m), 3.43 (2H, q), 3.45-3.52 (1H, m), 3.61-3.65 (1H, m), 3.76 (1H, d), 3.95-3.99 (1H, m), 4.19-4.22 (1H, m), 4.56 (1H, s), 6.44 (1H, d), 6.78 (1H, s), 7.50-7.52 (2H, m), 8.18-8.20 (2H, m), 8.54 (1H, s).

LCMS Spectrum: m/z (ESI+)(M+H)+=486; HPLC tR=1.96 min.

mTOR Kinase Assay (Echo): 0.00165 μM

The following compounds were prepared in an analogous fashion from phenyl N-[4-[4-(1-ethylsulfonylcyclopropyl)-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 11a | 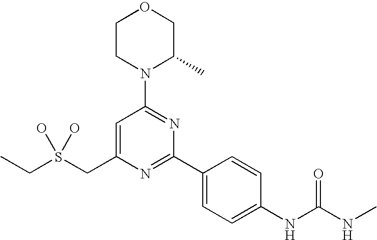 | 1-[4-[4-(1-ethylsulfonylcyclopropyl)-6-[(3S)-3-methylmorpholin-4-yl]pyrimidin-2-yl]phenyl]-3-methyl-urea | 460 | 1.79 |
| 11b | 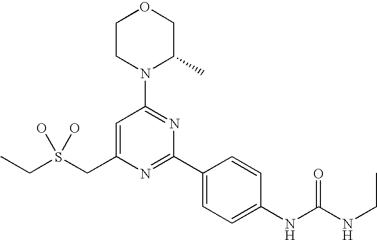 | 1-ethyl-3-[4-[4-(1-ethylsulfonylcyclopropyl)-6-[(3S)-3-methylmorpholin-4-yl]pyrimidin-2-yl]phenyl]urea | 474 | 1.93 |
| 11c | 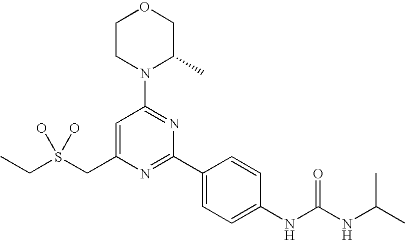 | 1-[4-[4-(1-ethylsulfonylcyclopropyl)-6-[(3S)-3-methylmorpholin-4-yl]pyrimidin-2-yl]phenyl]-3-propan-2-yl-urea | 488 | 2.11 |
| 11d | 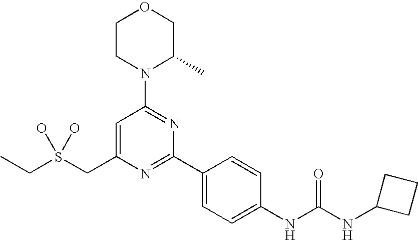 | 3-cyclobutyl-1-[4-[4-(1-ethylsulfonylcyclopropyl)-6-[(3S)-3-methylmorpholin-4-yl]pyrimidin-2-yl]phenyl]urea | 500 | 2.19 |
| 11e | 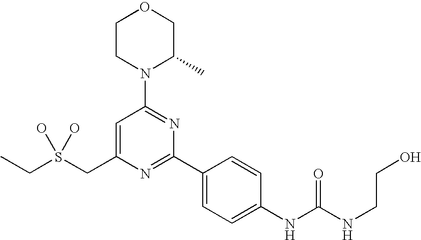 | 1-[4-[4-(1-ethylsulfonylcyclopropyl)-6-[(3S)-3-methylmorpholin-4-yl]pyrimidin-2-yl]phenyl]-3-(2-hydroxyethyl)urea | 490 | 1.63 |
| 11f | 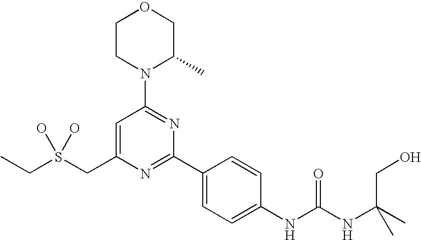 | 1-[4-[4-(1-ethylsulfonylcyclopropyl)-6-[(3S)-3-methylmorpholin-4-yl]pyrimidin-2-yl]phenyl]-3-(1-hydroxy-2-methyl-propan-2-yl)urea | 518 | 1.92 |
| 11g | 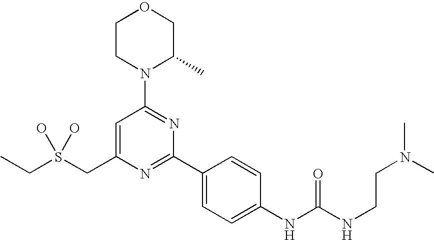 | 3-(2-dimethylaminomethyl)-1-[4-[4-(1-ethylsulfonylcyclopropyl)-6-[(3S)-3-methylmorpholin-4-yl]pyrimidin-2-yl]phenyl]urea | 517 | 1.90 |
| 11h | 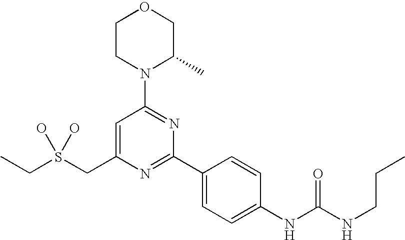 | 3-[4-[4-(1-ethylsulfonylcyclopropyl)-6-[(3S)-3-methylmorpholin-4-yl]pyrimidin-2-yl]phenyl]-1-propyl-urea | 488 | 2.11 |
| 11i | 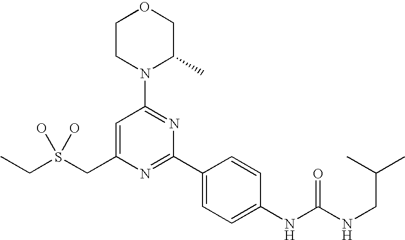 | 1-[4-[4-(1-ethylsulfonylcyclopropyl)-6-[(3S)-3-methylmorpholin-4-yl]pyrimidin-2-yl]phenyl]-3-(2-methylpropyl)urea | 502 | 2.29 |
| 11j | 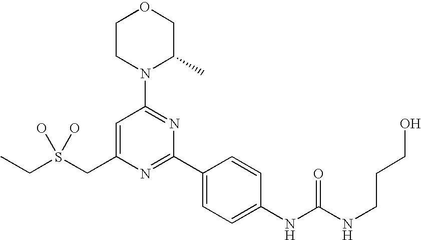 | 1-[4-[4-(1-ethylsulfonylcyclopropyl)-6-[(3S)-3-methylmorpholin-4-yl]pyrimidin-2-yl]phenyl]-3-(3-hydroxypropyl)urea | 504 | 1.68 |
| 11k | 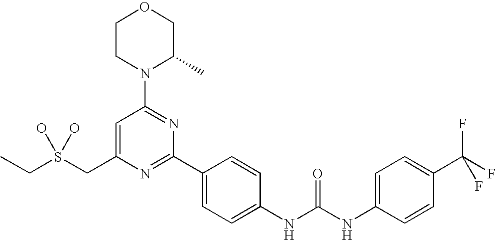 | 1-[4-[4-(1-ethylsulfonylcyclopropyl)-6-[(3S)-3-methylmorpholin-4-yl]pyrimidin-2-yl]phenyl]-3-[4-(trifluoromethyl)phenyl]urea | 590 | 2.80 |
| 11l | 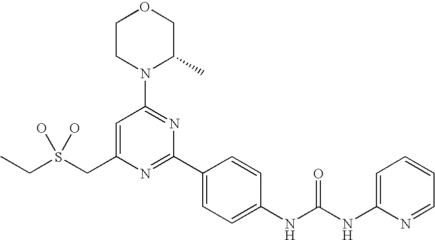 | 1-[4-[4-(1-ethylsulfonylcyclopropyl)-6-[(3S)-3-methylmorpholin-4-yl]pyrimidin-2-yl]phenyl]-3-pyridin-2-yl-urea | 523 | 2.37 |
| 11m | 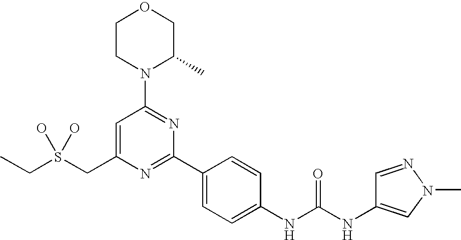 | 1-[4-[4-(1-ethylsulfonylcyclopropyl)-6-[(3S)-3-methylmorpholin-4-yl]pyrimidin-2-yl]phenyl]-3-(1-methylpyrazol-4-yl)urea | 526 | 1.84 |

### Example 11m can also be prepared in an analogous fashion but using DMA as the solvent and stirring at 50° C. for 18 hours.

### EXAMPLE 11a

¹H NMR (400.13 MHz, DMSO-d₆) δ 1.23 (3H, d), 1.32 (3H, t), 1.55 (2H, t), 1.62-1.64 (2H, m), 2.67 (3H, t), 3.20-3.24 (1H, m), 3.43 (2H, q), 3.45-3.52 (1H, m), 3.61-3.65 (1H, m), 3.76 (1H, d), 3.95-3.99 (1H, m), 4.19-4.22 (1H, m), 4.55 (1H, s), 6.07 (1H, d), 6.77 (1H, s), 7.49-7.52 (2H, m), 8.17-8.19 (2H, m), 8.74 (1H, s).

mTOR Kinase Assay (Echo): 0.00418 μM

### EXAMPLE 11b

¹H NMR (400.13 MHz, DMSO-d₆) δ 1.07 (3H, t), 1.23 (3H, d), 1.32 (3H, t), 1.55 (2H, t), 1.62-1.65 (2H, m), 3.09-3.16 (2H, m), 3.20-3.24 (1H, m), 3.43 (2H, q), 3.45-3.52 (1H, m), 3.61-3.65 (1H, m), 3.76 (1H, d), 3.95-3.99 (1H, m), 4.20 (1H, d), 4.56 (1H, s), 6.16 (1H, t), 6.77 (1H, s), 7.48-7.52 (2H, m), 8.17-8.19 (2H, m), 8.66 (1H, s).

mTOR Kinase Assay (Echo): 0.00333 μM

### EXAMPLE 11c

¹H NMR (400.13 MHz, DMSO-d₆) δ 1.11 (6H, d), 1.23 (3H, d), 1.32 (3H, t), 1.55 (2H, t), 1.60-1.65 (2H, m), 3.20-3.24 (1H, m), 3.43 (2H, q), 3.45-3.52 (1H, m), 3.61-3.65 (1H, m), 3.77 (1H, d), 3.80 (1H, m), 3.95-3.99 (1H, m), 4.19-4.22 (1H, m), 4.55 (1H, s), 6.07 (1H, d), 6.77 (1H, s), 7.47-7.50 (2H, m), 8.17-8.19 (2H, m), 8.53 (1H, s).

mTOR Kinase Assay (Echo): 0.0247 μM

### EXAMPLE 11d

¹H NMR (400.13 MHz, DMSO-d₆) δ 1.23 (3H, d), 1.32 (3H, t), 1.53-1.58 (2H, m), 1.62-1.64 (2H, m), 1.81-1.89 (3H, m), 2.18-2.24 (3H, m), 3.20-3.24 (1H, m), 3.43 (2H, q), 3.48-3.51 (1H, m), 3.61-3.65 (1H, m), 3.76 (1H, d), 3.95-3.99 (1H, m), 4.16 (2H, m), 4.55 (1H, s), 6.46-6.48 (1H, m), 6.78 (1H, s), 7.46-7.50 (2H, m), 8.18 (2H, d), 8.56 (1H, s).

mTOR Kinase Assay (Echo): 0.00642 μM

### EXAMPLE 11e

¹H NMR (400.13 MHz, DMSO-d₆) δ 1.23 (3H, d), 1.32 (3H, t), 1.55 (2H, t), 1.62-1.64 (2H, m), 3.18 (1H, q), 3.20 (2H, d), 3.40-3.51 (2H, m), 3.46 (1H, d), 3.42-3.52 (2H, m), 3.61-3.65 (1H, m), 3.76 (1H, d), 3.95-3.99 (1H, m), 4.20 (1H, d), 4.56 (1H, s), 4.73 (1H, t), 6.26 (1H, t), 6.78 (1H, s), 7.48-7.50 (2H, m), 8.19 (2H, d), 8.80 (1H, s).

mTOR Kinase Assay (Echo): 0.00135 μM

### EXAMPLE 11f

¹H NMR (400.13 MHz, DMSO-d₆) δ 1.23 (3H, d), 1.24 (6H, s), 1.32 (3H, d), 1.53-1.58 (2H, m), 1.62-1.64 (2H, m), 3.17-3.24 (1H, m), 3.39 (2H, d), 3.45 (2H, q), 3.45-3.52 (1H, m), 3.61-3.65 (1H, m), 3.76 (1H, d), 3.95-3.99 (1H, m), 4.19-4.22 (1H, m), 4.54 (1H, s), 4.95 (1H, t), 6.01 (1H, s), 6.77 (1H, s), 7.44-7.48 (2H, m), 8.18 (2H, d), 8.74 (1H, s).

mTOR Kinase Assay (Echo): 0.00337 μM

### EXAMPLE 11g

¹H NMR (400.13 MHz, DMSO-d₆) δ 1.23 (3H, d), 1.32 (3H, t), 1.53-1.58 (2H, m), 1.62-1.64 (2H, m), 2.18 (6H, s), 2.34 (2H, t), 3.17-3.23 (3H, m), 3.43 (2H, q), 3.45-3.52 (1H, m), 3.61-3.65 (1H, m), 3.76 (1H, d), 3.95-3.99 (1H, m), 4.19 (1H, s), 4.56 (1H, s), 6.16 (1H, t), 6.77 (1H, s), 7.48-7.51 (2H, m), 8.17-8.19 (2H, m), 8.89 (1H, s).

mTOR Kinase Assay (Echo): 0.313 μM

### EXAMPLE 11h

¹H NMR (400.13 MHz, DMSO-d₆) δ 0.89 (3H, t), 1.23 (3H, d), 1.32 (3H, t), 1.41-1.50 (2H, m), 1.55 (2H, t), 1.62-1.65 (2H, m), 3.04-3.09 (2H, m), 3.17-3.24 (1H, m), 3.43 (2H, q), 3.45-3.52 (1H, m), 3.61-3.65 (1H, m), 3.76 (1H, d), 3.95-3.99 (1H, m), 4.20 (1H, d), 4.55 (1H, s), 6.21 (1H, t), 6.77 (1H, s), 7.48-7.51 (2H, m), 8.17-8.20 (2H, m), 8.65 (1H, s).

mTOR Kinase Assay (Echo): 0.00913 μM

### Example 11i

¹H NMR (400.13 MHz, DMSO-d₆) δ 0.89 (6H, d), 1.23 (3H, d), 1.32-1.38 (3H, m), 1.55 (2H, t), 1.62-1.65 (2H, m), 1.69-1.76 (1H, m), 2.94 (2H, t), 3.17-3.24 (1H, m), 3.43 (2H, q), 3.45-3.52 (1H, m), 3.61-3.65 (1H, m), 3.76 (1H, d), 3.95-3.99 (1H, m), 4.20 (1H, d), 4.56 (1H, s), 6.25 (1H, t), 6.78 (1H, s), 7.48-7.51 (2H, m), 8.18 (1H, t), 8.20 (1H, s), 8.64 (1H, s).

mTOR Kinase Assay (Echo): 0.0294 μM

### EXAMPLE 11j

¹H NMR (400.13 MHz, DMSO-d₆) δ 1.23 (3H, d), 1.32 (3H, t), 1.55 (2H, q), 1.58 (2H, m), 1.62-1.64 (2H, m), 3.15-3.19 (3H, m), 3.43 (2H, q), 3.46-3.47 (2H, m), 3.50 (1H, d), 3.61-3.65 (1H, m), 3.76 (1H, d), 3.95-3.99 (1H, m), 4.20 (1H, d), 4.47 (1H, t), 4.55 (1H, s), 6.21 (1H, t), 6.77 (1H, s), 7.48-7.51 (2H, m), 8.17-8.20 (2H, m), 8.71 (1H, s).

mTOR Kinase Assay (Echo): 0.0122 μM

### EXAMPLE 11k

¹H NMR (400.13 MHz, DMSO-d₆) δ 1.24 (3H, d), 1.33 (3H, t), 1.56 (2H, t), 1.63-1.66 (2H, m), 3.18-3.26 (1H, m), 3.44 (2H, q), 3.46-3.53 (1H, m), 3.62-3.66 (1H, m), 3.77 (1H, d), 3.96-4.00 (1H, m), 4.22 (1H, d), 4.57 (1H, s), 6.81 (1H, s), 7.58-7.60 (2H, m), 7.64-7.70 (4H, m), 8.27 (2H, d), 9.05 (1H, s), 9.14 (1H, s).

mTOR Kinase Assay (Echo): 0.00332 μM

### EXAMPLE 11l

¹H NMR (400.13 MHz, DMSO-d₆) δ 1.25 (3H, d), 1.34 (3H, t), 1.55-1.59 (2H, m), 1.63-1.66 (2H, m), 3.18-3.23 (1H, m), 3.45 (2H, q), 3.47-3.53 (1H, m), 3.62-3.66 (1H, m), 3.77 (1H, d), 3.96-4.00 (1H, m), 4.20-4.24 (1H, m), 4.57 (1H, s), 6.81 (1H, s), 7.02-7.05 (1H, m), 7.56 (1H, d), 7.65 (2H, d), 7.75-7.77 (1H, m), 8.26-8.31 (1H, m), 8.27-8.31 (2H, m), 9.45 (1H, d), 10.61 (1H, s).

mTOR Kinase Assay (Echo): 0.00356 μM

### EXAMPLE 11m

¹H NMR (400.13 MHz, DMSO-d₆) δ 1.24 (3H, d), 1.33 (3H, t), 1.56 (2H, t), 1.63-1.65 (2H, m), 3.17-3.25 (1H, m), 3.44 (2H, q), 3.46-3.52 (1H, m), 3.62-3.66 (1H, m), 3.75 (1H, s), 3.79 (3H, s), 3.96-4.00 (1H, m), 4.21 (1H, d), 4.56 (1H, s), 6.79 (1H, s), 7.38-7.39 (1H, m), 7.53-7.57 (2H, m), 7.76 (1H, s), 8.22 (2H, d), 8.39 (1H, s), 8.84 (1H, s).

mTOR Kinase Assay (Echo): 0.00437 μM

The preparation of phenyl N-[4-[4-(1-ethylsulfonylcyclopropyl)-6-[(3S)-3-methylmorpholin-4-yl]pyrimidin-2-yl]phenyl]carbamate is described below.

### Phenyl N-[4-[4-(1-ethylsulfonylcyclopropyl)-6-[(3S)-3-methylmorpholin-4-yl]pyrimidin-2-yl]phenyl]carbamate

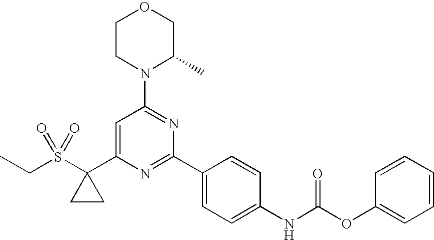

Phenyl chloroformate (0.566 mL, 4.50 mmol) was added dropwise to 4-[4-(1-ethylsulfonylcyclopropyl)-6-[(3S)-3-methylmorpholin-4-yl]pyrimidin-2-yl]aniline (1.81 g, 4.50 mmol) and sodium bicarbonate (0.567 g, 6.75 mmol) in dioxane (30 mL) under nitrogen. The resulting suspension was stirred at RT for 2 hours. The reaction mixture was evaporated to dryness and redissolved in ethyl acetate (200 mL) and washed with water (200 mL). The organic layer was dried (MgSO₄), filtered and evaporated to give the desired material as a white solid (2.36 g).

NMR Spectrum: ¹H NMR (400.13 MHz, DMSO-d₆) δ 1.24 (3H, d), 1.33 (3H, t), 1.55-1.59 (2H, m), 1.63-1.65 (2H, m), 3.21-3.25 (1H, m), 3.43-3.48 (2H, m), 3.41-3.52 (1H, m), 3.62-3.65 (1H, m), 3.77 (1H, d), 3.96-3.99 (1H, m), 4.21 (1H, s), 4.57 (1H, s), 6.82 (1H, s), 7.24-7.30 (3H, m), 7.43-7.47 (2H, m), 7.64 (2H, d), 8.27-8.30 (2H, m), 10.44 (1H, s)

LCMS Spectrum: m/z (ESI+) (M+H)+=523; HPLC tR=2.83 min.

### 4-[4-(1-Ethylsulfonylcyclopropyl)-6-[(3S)-3-methylmorpholin-4-yl]pyrimidin-2-yl]aniline

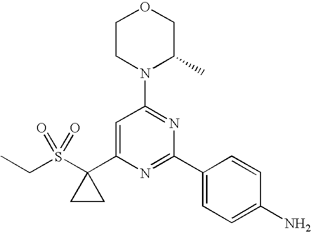

Dichlorobis(triphenylphosphine)palladium(II) (0.317 g, 0.45 mmol) was added to a degassed solution of 2-chloro-4-(1-ethylsulfonylcyclopropyl)-6-[(3S)-3-methylmorpholin-4-yl]pyrimidine (1.56 g, 4.51 mmol), 4-(4,4,5,5-tetramethyl-1,3,2-dioxaborolan-2-yl)aniline (1.235 g, 5.64 mmol) and sodium carbonate (11.28 ml, 22.55 mmol) in a mixture of 18% DMF in DME:water:ethanol (7:3:2) (20 mL). The resulting solution was stirred at 85° C. for 30 minutes. The reaction mixture was concentrated and partitioned between DCM (100 mL) and water (100 mL). The organic layer was dried (MgSO₄), filtered and evaporated to afford crude product. The crude product was purified by flash silica chromatography, elution gradient 0 to 2.5% methanol in DCM, to give the desired material as a brown oil (2.15 g).

NMR Spectrum: ¹H NMR (400.13 MHz, DMSO-d₆) δ 1.22 (3H, d), 1.31 (3H, t), 1.53 (2H, m), 1.60-1.62 (2H, m), 3.17-3.21 (1H, m), 3.37-3.47 (2H, m), 3.50 (1H, m), 3.60-3.64 (1H, m), 3.75 (1H, d), 3.94-3.98 (1H, m), 4.15-4.19 (1H, m), 4.51-4.53 (1H, m), 5.55 (2H, d), 6.60-6.62 (2H, m), 6.67 (1H, s), 8.00-8.04 (2H, m)

LCMS Spectrum: m/z (ESI+) (M+H)+=403; HPLC tR=2.14 min.

### 2-Chloro-4-(1-ethylsulfonylcyclopropyl)-6-[(3S)-3-methylmorpholin-4-yl]pyrimidine

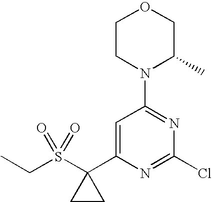

An aqueous sodium hydroxide solution (25.4 mL, 254.1 mmol) was added to tetrabutylammonium bromide (0.328 g, 1.02 mmol), 1,2-dibromoethane (0.876 mL, 10.16 mmol) and 2-chloro-4-(ethylsulfonylmethyl)-6-[(3S)-3-methylmorpholin-4-yl]pyrimidine (3.25 g, 10.16 mmol) in DCM (75 mL). The resulting mixture was stirred at 40° C. for 4 hours. The reaction mixture was diluted with DCM (50 mL) and washed with water (50 mL). The organic layer was dried (MgSO₄), filtered and evaporated to afford crude product. The crude product was purified by flash silica chromatography, elution gradient 0 to 40% ethyl acetate in DCM, to give the desired material as a white solid (1.56 g).

NMR Spectrum: ¹H NMR (400.13 MHz, DMSO-d₆) δ 1.17 (3H, d), 1.23 (3H, s), 1.49-1.52 (2H, m), 1.55-1.62 (2H, m), 3.18-3.23 (1H, m), 3.35 (2H, t), 3.41-3.46 (1H, m), 3.56-3.60 (1H, m), 3.72 (1H, d), 3.91-3.95 (1H, m), 4.15-4.19 (1H, m), 4.40 (1H, s), 6.93 (1H, s)

LCMS Spectrum: m/z (ESI+) (M+H)+=346; HPLC 1.97 tR=min.

### 2-Chloro-4-(ethylsulfonylmethyl)-6-[(3S)-3-methylmorpholin-4-yl]pyrimidine

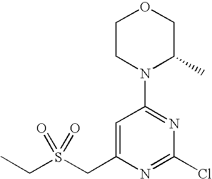

Ethane sulfinic acid sodium salt (3.94 g, 33.94 mmol) was added in one portion to 2-chloro-4-(iodomethyl)-6-[(3S)-3-methylmorpholin-4-yl]pyrimidine (12.0 g, 33.94 mmol) in acetonitrile (250 mL) at RT. The resulting suspension was stirred at 80° C. for 16 hours. The reaction mixture was evaporated to dryness and the residue partitioned between DCM (250 mL), and water (200mL). The organic layer was dried (MgSO₄), filtered and evaporated to afford crude product. The crude product was purified by flash silica chromatography, elution gradient 0 to 40% ethyl acetate in DCM, to give the desired material as a yellow solid (5.94 g).

NMR Spectrum: ¹H NMR (400.13 MHz, DMSO-d₆) δ 1.23 (3H, m), 1.28 (3H, t), 3.22 (2H, d), 3.32 (1H, s), 3.42-3.49 (1H, m), 3.58-3.62 (1H, m), 3.73 (1H, d), 3.92-3.96 (2H, m), 4.25-4.31 (1H, m), 4.43 (2H, s), 6.92 (1H, s)

LCMS Spectrum: m/z (ESI+) (M+H)+=320; HPLC tR=1.46 min.

The preparation of 2-chloro-4-(iodomethyl)-6-[(3S)-3-methylmorpholin-4-yl]pyrimidine was described earlier.

### EXAMPLE 12

### 3-Cyclopropyl-1-[4-[4-(1-methylsulfonylcyclopropyl)-6-morpholin-4-ylpyrimidin-2-yl]phenyl]urea

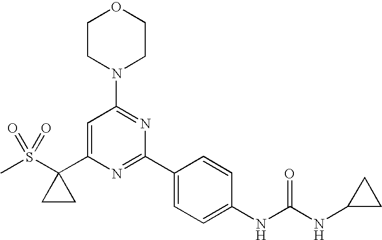

To a solution of phenyl N-[4-[4-(1-methylsulfonylcyclopropyl)-6-morpholin-4-ylpyrimidin-2-yl]phenyl]carbamate (150 mg, 0.29 mmol) in DMF (2 mL) was added triethylamine (0.127 mL, 0.91 mmol) followed by cyclopropylamine (0.106 mL, 1.52 mmol) and the reaction heated at 50° C. for 20 hours. The crude product was purified by preparative HPLC, using decreasingly polar mixtures of water (containing 1% NH3) and MeCN as eluents, to give the desired material as a white solid (80 mg).

NMR Spectrum: ¹H NMR (400.13 MHz, DMSO-d₆) δ 0.40-0.44 (2H, m), 0.64-0.66 (2H, m), 1.54-1.57 (1H, m), 1.55 (1H, d), 1.67 (1H, d), 1.65-1.68 (1H, m), 2.60 (1H, m), 3.30 (3H, s), 3.72 (8H, s), 6.43 (1H, d), 6.81 (1H, s), 7.50-7.52 (2H, m), 8.19-8.22 (2H, m), 8.55 (1H, s)

LCMS Spectrum: m/z (ESI+)(M+H)+=458; HPLC tR=1.44 min.

mTOR Kinase Assay (Echo): 0.00421 μM

The following compounds were made in an analogous fashion from either phenyl N-[4-[4-(1-methylsulfonylcyclopropyl)-6-morpholin-4-ylpyrimidin-2-yl]phenyl]carbamate, phenyl N-[4-[4-(1-methylsulfonylcyclopentyl)-6-morpholin-4-ylpyrimidin-2-yl]phenyl]carbamate or phenyl N-[4-[4-( 1-methylsulfonylcyclobutyl)-6-morpholin-4-ylpyrimidin-2-yl]phenyl]carbamate and the appropriate amines.

| | | | LCMS | Retention |
|---|---|---|---|---|
| Example | Structure | NAME | MH+ | time (min) |
| 12a | 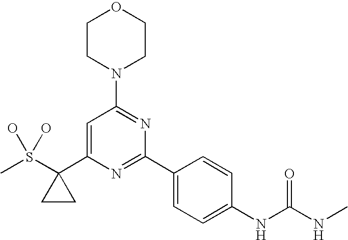 | 3-methyl-1-[4-[4-(1-methylsulfonylcyclopropyl)-6-morpholin-4-ylpyrimidin-2-yl]phenyl]urea | 432.5 | 1.27 |
| 12b | 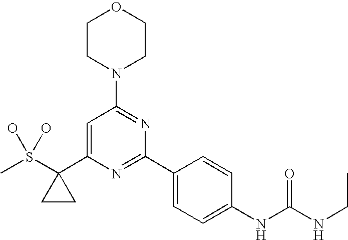 | 3-ethyl-1-[4-[4-(1-methylsulfonylcyclopropyl)-6-morpholin-4-ylpyrimidin-2-yl]phenyl]urea | 446.5 | 1.39 |
| 12c | 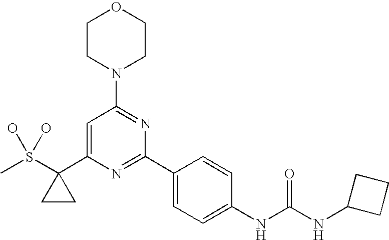 | 3-cyclobutyl-1-[4-[4-(1-methylsulfonylcyclopropyl)-6-morpholin-4-ylpyrimidin-2-yl]phenyl]urea | 471.5 | 1.73 |
| 12d | 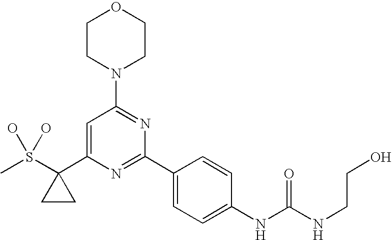 | 3-(2-hydroxyethyl)-1-[4-[4-(1-methylsulfonylcyclopropyl)-6-morpholin-4-ylpyrimidin-2-yl]phenyl]urea | 462.5 | 1.15 |
| 12e | 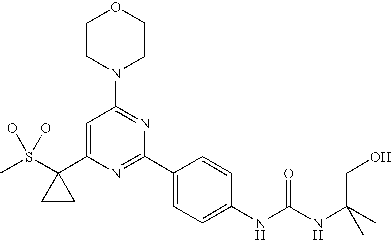 | 3-(1-hydroxy-2-methylpropan-2-yl)-1-[4-[4-(1-methylsulfonylcyclopropyl)-6-morpholin-4-ylpyrimidin-2-yl]phenyl]urea | 490.5 | 1.43 |
| 12f | 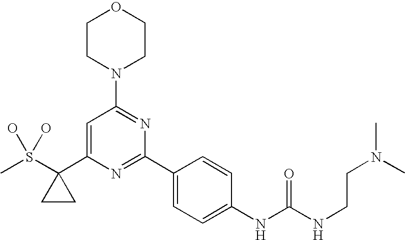 | 3-(2-dimethylaminoethyl)-1-[4-[4-(1-methylsulfonylcyclopropyl)-6-morpholin-4-ylpyrimidin-2-yl]phenyl]urea | 489.5 | 1.05 |
| 12g | 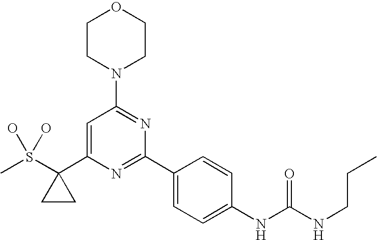 | 1-[4-[4-(1-methylsulfonylcyclopropyl)-6-morpholin-4-ylpyrimidin-2-yl]phenyl]-3-propylurea | 460.5 | 1.6 |
| 12h | 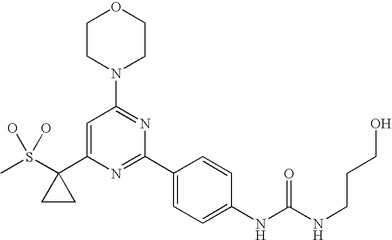 | 3-(3-hydroxypropyl)-1-[4-[4-(1-methylsulfonylcyclopropyl)-6-morpholin-4-ylpyrimidin-2-yl]phenyl]urea | 476.5 | 1.18 |
| 12i | 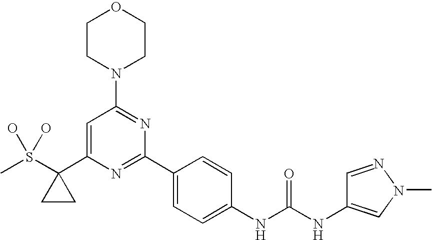 | 3-(1-methylpyrazol-4-yl)-1-[4-[4-(1-methylsulfonylcyclopropyl)-6-morpholin-4-ylpyrimidin-2-yl]phenyl]urea | 498.5 | 1.38 |
| 12j* | 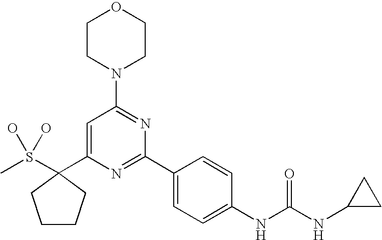 | 3-cyclopropyl-1-[4-[4-(1-methylsulfonylcyclopentyl)-6-morpholin-4-ylpyrimidin-2-yl]phenyl]urea | 486 | 2.04 |
| 12k* | 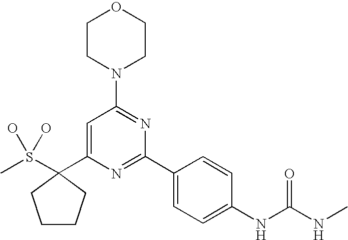 | 3-methyl-1-[4-[4-(1-methylsulfonylcyclopentyl)-6-morpholin-4-ylpyrimidin-2-yl]phenyl]urea | 460 | 1.88 |
| 12l* | 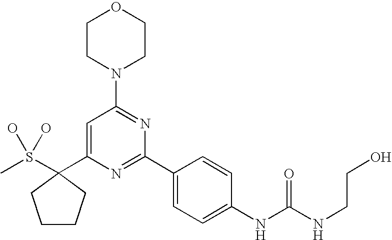 | 3-(2-hydroxyethyl)-1-[4-[4-(1-methylsulfonylcyclopentyl)-6-morpholin-4-ylpyrimidin-2-yl]phenyl]urea | 490 | 1.73 |
| 12m* | 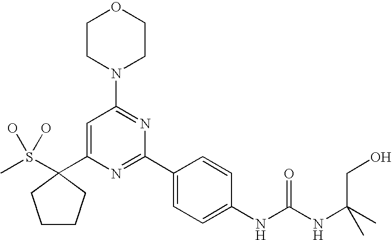 | 3-(1-hydroxy-2-methylpropan-2-yl)-1-[4-[4-(1-methylsulfonylcyclopentyl)-6-morpholin-4-ylpyrimidin-2-yl]phenyl]urea | 518 | 2.00 |
| 12n* | 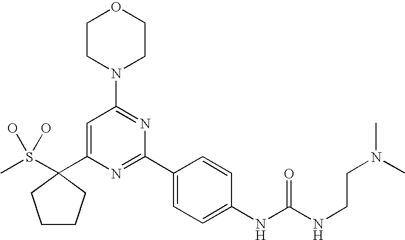 | 3-(2-dimethylaminoethyl)-1-[4-[4-(1-methylsulfonylcyclopentyl)-6-morpholin-4-ylpyrimidin-2-yl]phenyl]urea | 517 | 1.98 |
| 12o* | 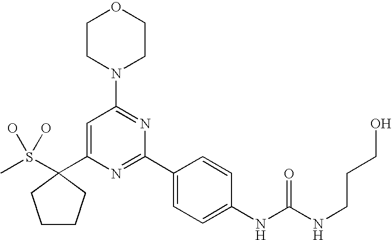 | 3-(3-hydroxypropyl)-1-[4-[4-(1-methylsulfonylcyclopentyl)-6-morpholin-4-ylpyrimidin-2-yl]phenyl]urea | 504 | 1.77 |
| 12p** | 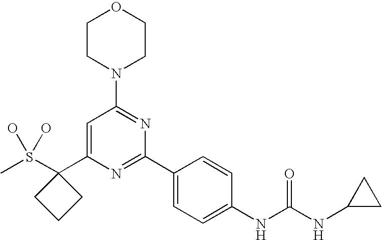 | 3-cyclopropyl-1-[4-[4-(1-methylsulfonylcyclobutyl)-6-morpholin-4-ylpyrimidin-2-yl]phenyl]urea | 472 | 2.06 |
| 12q** | 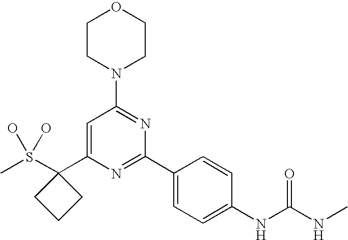 | 3-methyl-1-[4-[4-(1-methylsulfonylcyclobutyl)-6-morpholin-4-ylpyrimidin-2-yl]phenyl]urea | 446 | 1.88 |
| 12r** | 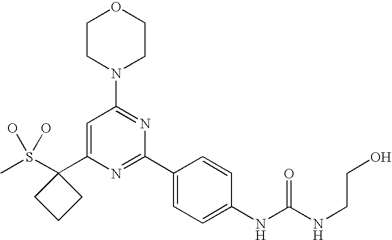 | 3-(2-hydroxyethyl)-1-[4-[4-(1-methylsulfonylcyclobutyl)-6-morpholin-4-ylpyrimidin-2-yl]phenyl]urea | 476 | 1.72 |
| 12s** | 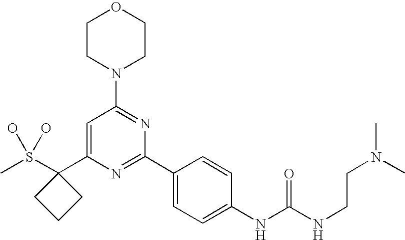 | 3-(2-dimethylaminoethyl)-1-[4-[4-(1-methylsulfonylcyclobutyl)-6-morpholin-4-ylpyrimidin-2-yl]phenyl]urea | 503 | 1.92 |
| 12t** | 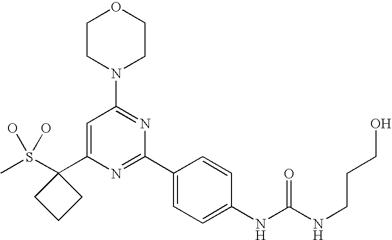 | 3-(3-hydroxypropyl)-1-[4-[4-(1-methylsulfonylcyclopentyl)-6-morpholin-4-ylpyrimidin-2-yl]phenyl]urea | 490 | 1.77 |

*Reaction stirred at 55° C. for 6 hours
**Reaction stirred at 40° C. for 6 hours

The crude material for Example 12 can also be purified either by chromatography on silica, eluting with 50-80% ethyl acetate in isohexane, or by dissolving the material in ethyl acetate and allowing the desired material to precipitate from solution on stirring.

### EXAMPLE 12a

¹H NMR (400.13 MHz, DMSO-d₆) δ 1.53-1.57 (1H, m), 1.55-1.56 (1H, m), 1.67 (1H, d), 1.65-1.68 (1H, m), 2.66 (3H, d), 3.30 (3H, s), 3.72 (8H, s), 6.06 (1H, d), 6.81 (1H, s), 7.49-7.52 (2H, m), 8.19-8.21 (2H, m), 8.75 (1H, s).

mTOR Kinase Assay (Echo): 0.00167 μM

### EXAMPLE 12b

¹H NMR (400.13 MHz, DMSO-d₆) δ 1.07 (3H, t), 1.54-1.57 (1H, m), 1.55 (1H, d), 1.67 (1H, d), 1.65-1.68 (1H, m), 3.11-3.14 (2H, m), 3.30 (3H, s), 3.72 (8H, s), 6.16 (1H, s), 6.81 (1H, s), 7.49-7.51 (2H, m), 8.19-8.21 (2H, m), 8.67 (1H, s).

mTOR Kinase Assay (Echo): 0.00271 μM

### EXAMPLE 12c

¹H NMR (400.13 MHz, DMSO-d₆) 6 1.53-1.57 (2H, m), 1.55 (2H, d), 1.59-1.67 (2H, m), 1.82-1.85 (1H, m), 1.89 (1H, t), 2.18-2.25 (2H, m), 3.30 (8H, s), 4.14 (1H, d), 6.46 (1H, d), 6.81 (1H, s), 7.47-7.49 (2H, m), 8.20 (2H, d), 8.57 (1H, s).

mTOR Kinase Assay (Echo): 0.00152 μM

### EXAMPLE 12d

¹H NMR (400.13 MHz, DMSO-d₆) 6 1.54-1.57 (1H, m), 1.55-1.56 (1H, m), 1.67 (1H, d), 1.65-1.68 (1H, m), 3.18 (2H, q), 3.30 (3H, s), 3.46 (2H, q), 3.72 (8H, s), 4.73 (1H, t), 6.25 (1H, t), 6.81 (1H, s), 7.48-7.50 (2H, m), 8.19-8.22 (2H, m), 8.81 (1H, s).

mTOR Kinase Assay (Echo): 0.00155 μM

### EXAMPLE 12e

¹H NMR (400.13 MHz, DMSO-d₆) δ 1.24 (6H, s), 1.54-1.57 (1H, m), 1.55 (1H, d), 1.67 (1H, d), 1.65-1.68 (1H, m), 3.30 (3H, s), 3.39 (2H, d), 3.72 (8H, s), 4.95 (1H, t), 6.00 (1H, s), 6.80 (1H, s), 7.44-7.47 (2H, m), 8.18-8.20 (2H, m), 8.74 1H, s).

mTOR Kinase Assay (Echo): 0.00459 μM

### EXAMPLE 12f

¹H NMR (400.13 MHz, DMSO-d₆) δ 1.54-1.57 (1H, m), 1.55-1.56 (1H, m), 1.67 (1H, d), 1.65-1.68 (1H, m), 2.18 (6H, s), 2.34 (2H, t), 3.20 (2H, t), 3.30 (3H, s), 3.72 (8H, s), 6.16 (1H, t), 6.80 (1H, s), 7.47-7.51 (2H, m), 8.20 (2H, d), 8.90 (1H, s).

mTOR Kinase Assay (Echo): 0.0605 μM

### EXAMPLE 12g

¹H NMR (400.13 MHz, DMSO-d₆) δ 0.89 (3H, t), 1.41-1.50 (2H, m), 1.54-1.57 (1H, m), 1.55 (1H, d), 1.67 (1H, d), 1.65-1.68 (1H, m), 3.04-3.09 (2H, m), 3.30 (3H, s), 3.72 (8H, s), 6.20 (1H, t), 6.81 (1H, s), 7.48-7.51 (2H, m), 8.18-8.22 (2H, m), 8.66 (1H, s).

mTOR Kinase Assay (Echo): 0.00273 μM

### EXAMPLE 12h

¹H NMR (400.13 MHz, DMSO-d₆) δ 1.53-1.58 (2H, m), 1.61 (2H, d), 1.65-1.68 (2H, m), 3.17 (2H, d), 3.30 (3H, s), 3.45-3.48 (2H, m), 3.72 (8H, s), 4.47 (1H, t), 6.20 (1H, s), 6.81 (1H, s), 7.48-7.51 (2H, m), 8.19-8.21 (2H, m), 8.72 (1H, s).

mTOR Kinase Assay (Echo): 0.00392 μM

### EXAMPLE 12i

¹H NMR (400.13 MHz, DMSO-d₆) δ 1.54-1.58 (1H, m), 1.56-1.56 (1H, m), 1.67 (2H, t), 3.30 (3H, s), 3.72 (8H, s), 3.79 (3H, s), 6.82 (1H, s), 7.38-7.56 (2H, m), 7.76 (1H, s), 8.23-8.25 (2H, m), 8.38 (1H, s), 8.84 (1H, s).

mTOR Kinase Assay (Echo): 0.000771 μM

### EXAMPLE 12j

¹H NMR (400.13 MHz, DMSO-d₆) δ 0.40-0.44 (2H, m), 0.63-0.67 (2H, m), 1.58 (2H, t), 1.81 (2H, d), 2.39-2.47 (2H, m), 2.57 (1H, t), 2.74 (2H, t), 2.91 (3H, s), 3.73 (8H, s), 6.42 (1H, d), 6.84 (1H, s), 7.49-7.53 (2H, m), 8.22-8.26 (2H, m), 8.54 (1H, s).

mTOR Kinase Assay (Echo): 0.00167 μM

### EXAMPLE 12k

¹H NMR (400.13 MHz, DMSO-d₆) δ 1.56-1.57 (2H, m), 1.80-1.82 (2H, m), 2.42 (2H, d), 2.46 (2H, d), 2.67 (3H, t), 2.91 (3H, s), 3.73 (8H, s), 6.06 (1H, d), 6.84 (1H, s), 7.49-7.52 (2H, m), 8.22-8.25 (2H, m), 8.74 (1H, s).

mTOR Kinase Assay (Echo): 0.0043 μM

### EXAMPLE 12l

¹H NMR (400.13 MHz, DMSO-d₆) δ 1.58 (2H, t), 1.81 (2H, d), 2.41-2.44 (2H, m), 2.73 (2H, d), 2.91 (3H, s), 3.18 (2H, q), 3.46 (2H, q), 3.73 (8H, t), 6.25 (1H, s), 6.84 (1H, s), 7.48-7.50 (2H, m), 8.23-8.25 (2H, m), 8.81 (1H, s).

mTOR Kinase Assay (Echo): 0.00138 μM

### EXAMPLE 12m

¹H NMR (400.13 MHz, DMSO-d₆) δ 1.24 (6H, s), 1.58 (2H, t), 1.81 (2H, d), 2.41-2.44 (2H, m), 2.71-2.75 (2H, m), 2.91 (3H, s), 3.39 (2H, d), 3.73 (8H, s), 4.95 (1H, t), 6.00 (1H, s), 6.84 (1H, s), 7.44-7.47 (2H, m), 8.22-8.24 (2H, m), 8.74 (1H, s).

mTOR Kinase Assay (Echo): 0.0101 μM

### EXAMPLE 12n

¹H NMR (400.13 MHz, DMSO-d₆) δ 1.56-1.57 (2H, m), 1.81 (2H, d), 2.18 (6H, s), 2.34 (2H, t), 2.39-2.46 (2H, m), 2.73 (2H, q), 2.91 (3H, s), 3.19 (2H, q), 3.73 (8H, s), 6.15 (1H, t), 6.84 (1H, s), 7.48-7.50 (2H, m), 8.23-8.25 (2H, m), 8.90 (1H, s).

mTOR Kinase Assay (Echo): 0.238 μM

### EXAMPLE 12o

¹H NMR (400.13 MHz, DMSO-d₆) δ 1.56-1.59 (2H, m), 1.57-1.63 (2H, m), 1.81 (2H, d), 2.39-2.46 (2H, m), 2.72-2.74 (2H, t), 2.91 (3H, s), 3.15-3.19 (2H,m), 3.45-3.50 (2H, m), 3.73 (8H, s), 4.47 (1H, t), 6.20 (1H, t), 6.84 (1H, s), 7.48-7.51 (2H, m), 8.22-8.25 (2H, m), 8.71 (1H, s).

mTOR Kinase Assay (Echo): 0.00509 μM

### EXAMPLE 12p

¹H NMR (400.13 MHz, DMSO-d₆) δ 0.41-0.43 (2H, m), 0.63-0.66 (2H, m), 1.90 (1H, m), 2.08 (1H, m), 2.33 (1H, t), 2.68 (1H, t), 2.80-2.82 (1H,m), 2.87 (3H, s), 2.90 (2H, m), 3.73 (8H, s), 6.43-6.44 (1H, m), 6.77 (1H, s), 7.49-7.51 (2H, m), 8.21-8.24 (2H, m), 8.56 (1H, s).

mTOR Kinase Assay (Echo): 0.0014 μM

### EXAMPLE 12q

¹H NMR (400.13 MHz, DMSO-d₆) δ 1.99-2.06 (1H, m), 2.09 (1H, m), 2.32-2.34 (3H, m), 2.87 (3H, s), 2.90 (2H, s), 2.93 (2H, s), 3.73 (8H, s), 6.06 (1H, d), 6.76 (1H, s), 7.50 (2H, d), 8.22 (2H, d), 8.74 (1H, s).

mTOR Kinase Assay (Echo): 0.00162 μM

### EXAMPLE 12r

¹H NMR (400.13 MHz, DMSO-d₆) δ 1.89 (1H, t), 2.07 (1H, d), 2.76-2.83 (2H, m), 2.87 (3H, s), 2.90-2.93 (2H, m), 3.18 (2H, m), 3.46 (2H, m), 3.73 (8H, s), 4.72 (1H, t), 6.25 (1H, t), 6.76 (1H, s), 7.48-7.50 (2H, m), 8.21-8.23 (2H, m), 8.81 (1H, s).

mTOR Kinase Assay (Echo): 0.000991 μM

### EXAMPLE 12s

¹H NMR (400.13 MHz, DMSO-d₆) δ 1.89 (1H, t), 2.07 (1H, t), 2.18 (6H, s), 2.34 (2H, t), 2.80 (2H, d), 2.87 (3H, s), 2.90 (2H, m), 3.19 (2H, t), 3.73 (8H, s), 6.16 (1H, s), 6.76 (1H, s), 7.47-7.50 (2H, m), 8.21-8.23 (2H, m), 8.90 (1H, s).

mTOR Kinase Assay (Echo): 0.134 μM

### EXAMPLE 12t

¹H NMR (400.13 MHz, DMSO-d₆) δ 1.58 (2H, t), 1.87-1.92 (1H, m), 2.06-2.10 (1H, m), 2.76-2.82 (2H, m), 2.87 (3H, s), 2.90-2.93 (2H, m), 3.14-3.49 (2H, m), 3.73 (8H, s), 4.47 (1H, t), 6.20 (1H, t), 6.76 (1H, s), 7.48-7.50 (2H, m), 8.21-8.23 (2H, m), 8.71 (1H, s).

mTOR Kinase Assay (Echo): 0.0366 μM

The preparation of phenyl N-[4-[4-(1-methylsulfonylcyclopropyl)-6-morpholin-4-ylpyrimidin-2-yl]phenyl]carbamate is described below:

### Phenyl N-[4-[4-(1-methylsulfonylcyclopropyl)-6-morpholin-4-ylpyrimidin-2-yl]phenyl]carbamate

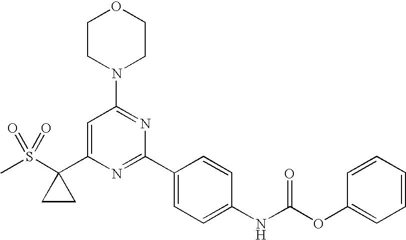

Phenyl chloroformate (O.4 mL, 3.18 mmol) was added dropwise to 4-(4-(1-(methylsulfonyl)cyclopropyl)-6-morpholinopyrimidin-2-yl)aniline (1.19 g, 3.18 mmol) and sodium bicarbonate (0.40 g, 4.77 mmol) in dioxane (30 mL) under nitrogen. The resulting suspension was stirred at RT for 2 hours. The reaction mixture was evaporated to dryness and redissolved in ethyl acetate (100 mL) and washed with water (100 mL). The organic layer was dried (MgSO₄), filtered and evaporated to afford the desired material as a yellow solid (1.68 g).

NMR Spectrum: ¹H NMR (400.13 MHz, DMSO-d₆) δ 1.57 (1H, d), 1.55-1.62 (1H, m), 1.68 (1H, d), 1.66-1.69 (1H, m), 3.40 (3H, s), 3.73 (8H, s), 6.86 (1H, s), 7.24-7.30 (3H, m), 7.43-7.47 (2H, m), 7.63-7.65 (2H, m), 8.29-8.31 (2H, m), 10.44 (1H, s)

LCMS Spectrum: m/z (ESI+) (M+H)+=495; HPLC tR=2.58 min.

### 4-[4-(1-Methylsulfonylcyclopropyl)-6-morpholin-4-ylpyrimidin-2-yl]aniline

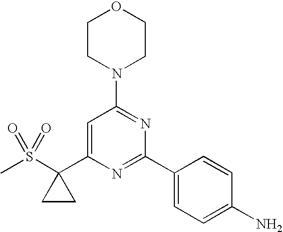

Dichlorobis(triphenylphosphine)palladium(II) (0.636 g, 0.91 mmol) was added to 2-chloro-4-(1-methylsulfonylcyclopropyl)-6-morpholin-4-ylpyrimidine (2.88 g, 9.06 mmol), 4-(4,4,5,5-tetramethyl-1,3,2-dioxaborolan-2-yl)aniline (2.482 g, 11.33 mmol) and sodium carbonate (22.66 mL, 45.31 mmol) in 18% DMF in 7:3:2 DME:Water:Ethanol (40mL). The resulting solution was stirred at 85° C. for 1 hour. The reaction mixture was concentrated, diluted with DCM (200 mL), and washed with water (200 mL). The organic layer was dried (MgSO₄), filtered and evaporated to afford crude product. The crude product was purified by flash silica chromatography, eluting with 0 to 2.5% methanol in DCM, to give the desired material as a yellow solid (1.19 g).

NMR Spectrum: ¹H NMR (400.13 MHz, DMSO-d₆) δ 1.51-1.53 (2H, m), 1.65-1.66 (2H, m), 3.40 (3H, s), 3.70 (8H, s), 5.56 (2H, d), 6.61 (2H, d), 6.70 (1H, s), 8.04 (2H, d)

LCMS Spectrum: m/z (ESI+) (M+H)+=375; HPLC tR=1.65 min.

### 2-Chloro-4-(1-methylsulfonylcyclopropyl)-6-morpholin-4-ylpyrimidine

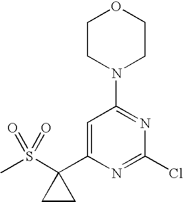

Sodium hydroxide (9.60 mL, 95.97 mmol) was added to 2-chloro-4-(methylsulfonylmethyl)-6-morpholin-4-yl-pyrimidine (2.80 g, 9.60 mmol), 1,2-dibromoethane (1.654 mL, 19.19 mmol) and tetrabutylammonium bromide (0.619 g, 1.92 mmol) in toluene (120 mL) at RT. The resulting solution was stirred at 60° C. for 3 hours. The reaction mixture was evaporated to dryness, redissolved in ethyl acetate (200 mL), and washed sequentially with water (200 mL) and saturated brine (100 mL). The organic layer was dried (MgSO₄), filtered and evaporated to afford crude product. The crude product was purified by flash silica chromatography, eluting with 0 to 2.5% methanol in DCM, to give the desired material as a yellow solid (2.88 g).

NMR Spectrum: ¹H NMR (400.13 MHz, DMSO-d₆) δ 1.49-1.51 (2H, m), 1.62-1.65 (2H, m), 3.19 (3H, s), 3.67 (8H, d), 6.96 (1H, s)

LCMS Spectrum: m/z (ESI+) (M+H)+=318; HPLC 1.37 tR=min.

### 2-Chloro-4-(methylsulfonylmethyl)-6-morpholin-4-yl-pyrimidine

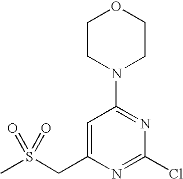

A suspension of 2,4-dichloro-6-(methylsulfonylmethyl)pyrimidine (10.56 g) in DCM (230 mL) was stirred magnetically (under nitrogen) and cooled to −5° C. Triethylamine (6.78 mL) was added followed by the dropwise addition of a solution of morpholine (3.85 mL) in DCM (30 mL) maintaining the reaction temperature below −5° C. The reaction was stirred at room temperature for 1 hour and then the organic mixture washed with water (300 mL). The organic phase was dried ( MgSO4), filtered and evaporated to a brown solid which was chromatographed on silica, eluting with 50% ethyl acetate in DCM, to give the desired material (6.81g) as a white solid.

NMR Spectrum: ¹H NMR (400.13 MHz, DMSO-d₆) δ 3.12 (3H, s), 3.63 (4H, s), 3.68-3.70 (4H, m), 4.45 (2H, s), 6.96 (1H, s)

Mass Spectrum: MH+ 292.

### 2,4-Dichloro-6-(methylsulfonylmethyl)pyrimidine

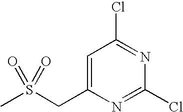

6-(Methylsulfonylmethyl)-1H-pyrimidine-2,4-dione (132 g, 0.65 mol) was added to phosphorus oxychloride (1.2 L) and the mixture heated to reflux for 16 hours, then cooled to room temperature. The excess phosphorus oxychloride was removed in vacuo, the residue azeotroped with toluene (2×500 mL) and dissolved in dichloromethane. This mixture was then poured slowly onto ice (4 L) and stirred for 20 minutes, then extracted with dichloromethane (3×1 L) (the insoluble black material was filtered off and discarded) and ethyl acetate (2×1 L). The extracts were combined, dried, then evaporated to leave the desired material as a dark brown solid (51 g). The material was used without further purification.

NMR Spectrum: ¹H NMR (400.13 MHz, DMSO-d₆) δ 3.13 (s, 3H), 4.79 (s, 2H), 7.87 (s, 1H)

LCMS Spectrum: m/z (ESI+) (M+H)+=239; HPLC 1.21 tR=min.

### 6-(Methylsulfonylmethyl)-1H-pyrimidine-2,4-dione

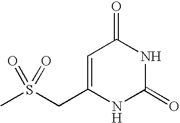

6-(Chloromethyl)-1H-pyrimidine-2,4-dione (175 g, 1.09 mol) was dissolved in DMF (2 L) and methanesulphinic acid sodium salt (133.5 g, 1.31 mol) was added. The reaction was heated to 125° C. for 2 hours then allowed to cool and the suspension filtered and concentrated in vacuo to give a yellow solid. The crude material was washed with water, filtered, then triturated with toluene. The solid was filtered then triturated with isohexane to leave the desired compound as a yellow solid (250 g). The material was used without further purification.

6-(Chloromethyl)-1H-pyrimidine-2,4-dione is a commercially available material.

The preparation of phenyl N-[4-[4-(1-methylsulfonylcyclopentyl)-6-morpholin-4-ylpyrimidin-2-yl]phenyl]carbamate is described below.

### Phenyl N-[4-[4-(1-methylsulfonylcyclopentyl)-6-morpholin-4-ylpyrimidin-2-yl]phenyl]carbamate

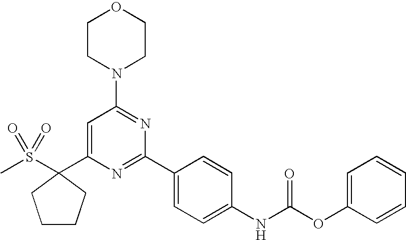

Phenyl chloroformate (0.541 mL, 4.30 mmol) was added dropwise to 4-[4-(1-methylsulfonylcyclopentyl)-6-morpholin-4-ylpyrimidin-2-yl]aniline (1.73 g, 4.30 mmol) and sodium bicarbonate (0.542 g, 6.45 mmol) in dioxane (50 mL) under nitrogen. The resulting suspension was stirred at RT for 1 hour. The reaction mixture was evaporated to dryness and partitioned between ethyl acetate (100 mL) and water (100 mL). The organic layer was dried (MgSO₄), filtered and evaporated to afford the desired material as a yellow solid (1.7 g).

NMR Spectrum: ¹H NMR (400.13 MHz, DMSO-d₆) δ 1.58 (2H, t), 1.82 (2H, d), 2.40-2.47 (2H, m), 2.73-2.75 (2H, m), 2.91 (3H, s), 3.74 (8H, s), 6.88 (1H, s), 7.24-7.30 (3H,l m), 7.43-7.47 (2H, m), 7.64 (2H, d), 8.33-8.35 (2H, m), 10.43 (1H, s), LCMS Spectrum: m/z (ESI+) (M+H)+=523; HPLC tR=2.90 min.

### 4-[4-(1-Methylsulfonylcyclopentyl)-6-morpholin-4-ylpyrimidin-2-yl]aniline

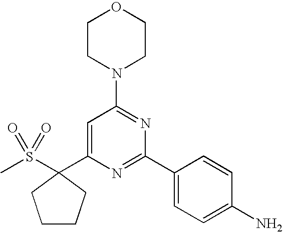

Dichlorobis(triphenylphosphine)palladium (II) (0.224 g, 0.32 mmol) was added to a solution of 2-chloro-4-(1-methylsulfonylcyclopentyl)-6-morpholin-4-ylpyrimidine (2.21 g, 6.39 mmol), 4-(4,4,5,5-tetramethyl-1,3,2-dioxaborolan-2-yl)aniline (1.75 g, 7.99 mmol) and aqueous sodium carbonate solution (15.98 mL, 31.95 mmol) in a solvent mixture of 18% DMF in 7:3:2 DME:Water:Ethanol (40 mL). The resulting solution was stirred at 85° C. for 1 hour. The reaction mixture was concentrated and diluted with DCM (200 mL) and washed with water (200 mL). The organic layer was dried (MgSO₄), filtered and evaporated to afford crude product. The crude product was purified by flash silica chromatography, elution gradient 0 to 2.5% methanol in DCM, to give the desired material as a yellow solid (1.73 g).

NMR Spectrum: ¹H NMR (400.13 MHz, DMSO-d₆) δ 1.57 (2H, t), 1.80 (2H, d), 2.37-2.44 (2H, m), 2.69-2.74 (2H, m), 2.90 (3H, s), 3.71 (8H, s), 5.55 (2H, d), 6.59-6.63 (2H, m), 6.74 (1H, s), 8.05-8.09 (2H, m)

LCMS Spectrum: m/z (ESI+) (M+H)+=403; HPLC tR=2.22 min.

### 2-Chloro-4-(1-methylsulfonylcyclopentyl)-6-morpholin-4-ylpyrimidine

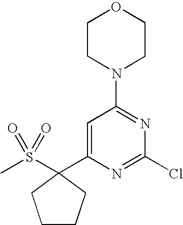

10N Sodium hydroxide solution (8.57 mL, 85.69 mmol) was added to 2-chloro-4-(methylsulfonylmethyl)-6-morpholin-4-yl-pyrimidine (2.50 g, 8.57 mmol), 1,4-dibromobutane (1.014 mL, 8.57 mmol) and tetrabutylammonium bromide (0.552 g, 1.71 mmol) in toluene (120 mL) at RT. The resulting solution was stirred at 60° C. for 4 hours. The reaction mixture was evaporated to dryness and the residue partitioned between ethyl acetate (200 mL) and water (200 mL). The organic layer was dried (MgSO₄), filtered and evaporated to afford crude product. The crude product was purified by flash silica chromatography, elution gradient 20 to 50% ethyl acetate in isohexane, to give the desired material as a yellow solid (2.215 g).

NMR Spectrum: ¹H NMR (400.13 MHz, DMSO-d₆) δ 1.51-1.58 (2H, m), 1.76-1.80 (2H, m), 2.33-2.40 (2H, m), 2.52-2.59 (2H, m), 2.89 (3H, s), 3.67 (8H, s), 6.96 (1H, s)

LCMS Spectrum: m/z (ESI+) (M+H)+=346; HPLC 2.12 tR=min.

The preparation of 2-chloro-4-(methylsulfonylmethyl)-6-morpholin-4-yl-pyrimidine was described earlier.

The preparation of phenyl N-[4-[4-(1-methylsulfonylcyclobutyl)-6-morpholin-4-ylpyrimidin-2-yl]phenyl]carbamate is described below.

### Phenyl N-[4-[4-(1-methylsulfonylcyclobutyl)-6-morpholin-4-ylpyrimidin-2-yl]phenyl]carbamate

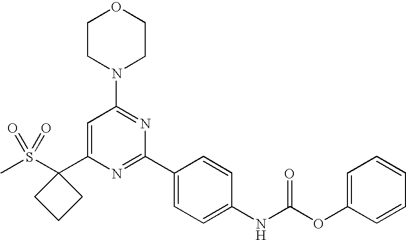

Phenyl chloroformate (0.084 mL, 0.67 mmol) was added dropwise to 4-[4-(1-methylsulfonylcyclobutyl)-6-morpholin-4-ylpyrimidin-2-yl]aniline (260 mg, 0.67 mmol) and sodium bicarbonate (84 mg, 1.00 mmol) in dioxane (20 mL) under nitrogen. The resulting suspension was stirred at RT for 2 hours. The reaction mixture was evaporated to dryness, redissolved in ethyl acetate (100 mL) and washed with water (100 mL). The organic layer was dried (MgSO₄), filtered and evaporated to afford the desired material as a cream solid (380 mg).

NMR Spectrum: ¹H NMR (400.13 MHz, DMSO-d₆) δ 1.91 (1H, d), 2.06 (1H, t), 2.80-2.84 (2H, m), 2.88 (3H, s), 2.93 (2H, d), 3.74 (8H, d), 6.80 (1H, s), 7.24-7.26 (2H, m), 7.25-7.30 (1H, m), 7.43-7.47 (2H, m), 7.63 (2H, d), 8.32 (2H, d), 10.43 (1H, s)

LCMS Spectrum: m/z (ESI+) (M+H)+=509; HPLC tR=2.77 min.

### 4-[4-(1-Methylsulfonylcyclobutyl)-6-morpholin-4-ylpyrimidin-2-yl]aniline

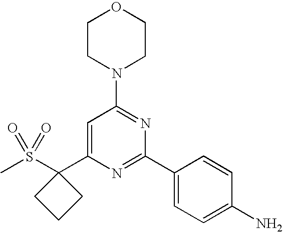

Dichlorobis(triphenylphosphine)palladium(II) (0.167 g, 0.24 mmol) was added to a degassed solution of 2-chloro-4-(1-methylsulfonylcyclobutyl)-6-morpholin-4-ylpyrimidine (0.790 g, 2.38 mmol), 4-(4,4,5,5-tetramethyl-1,3,2-dioxaborolan-2-yl)aniline (0.652 g, 2.98 mmol) and an aqueous solution of sodium carbonate (5.95 mL, 11.90 mmol) in a solvent mixture of 18% DMF in 7:3:2 DME:Water:Ethanol (40 mL). The resulting solution was stirred at 85° C. for 1 hour. The reaction mixture was concentrated, diluted with DCM (200 mL), and washed with water (200 mL). The organic layer was dried (MgSO₄), filtered and evaporated to afford crude product. The crude product was purified by flash silica chromatography, elution gradient 0 to 50% ethyl acetate in isohexane, to give the desired material as a white solid (0.22 g).

NMR Spectrum: ¹H NMR (400.13 MHz, DMSO-d₆) δ 1.89 (1H, d), 2.03-2.07 (1H, m), 2.74-2.81 (2H, m), 2.86 (3H, s), 2.88-2.93 (2H, m), 3.71 (8H, s), 5.54 (2H, d), 6.59-6.62 (2H, m), 6.66 (1H, s), 8.04-8.07 (2H, m)

LCMS Spectrum: m/z (ESI+) (M+H)+=389; HPLC tR=2.05 min.

### 2-Chloro-4-(1-methylsulfonylcyclobutyl)-6-morpholin-4-ylpyrimidine

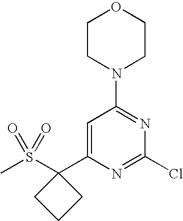

10N Sodium hydroxide solution (9.60 mL, 95.97 mmol) was added to 1,3-dibromopropane (0.979 mL, 9.60 mmol), 2-chloro-4-(methylsulfonylmethyl)-6-morpholin-4-yl-pyrimidine (2.80 g, 9.60 mmol) and tetrabutylammonium bromide (0.619 g, 1.92 mmol) in toluene (120 mL) at RT. The resulting solution was stirred at 60° C. for 18 hours. The reaction mixture was evaporated to dryness, redissolved in ethyl acetate (200 mL), and washed sequentially with water (200 mL) and saturated brine solution (100 mL). The organic layer was dried (MgSO₄), filtered and evaporated to afford crude product. The crude product was purified by flash silica chromatography, elution gradient 0 to 50% ethyl acetate in isohexane, to give the NMR Spectrum: ¹H NMR (400.13 MHz, DMSO-d₆) δ 1.87-1.89 (1H, m), 2.01 (1H, d), 2.66-2.73 (2H, m), 2.81-2.84 (2H, m), 2.86 (3H, s), 3.67 (8H, s), 6.88 (1H, s)

LCMS Spectrum: m/z (ESI+) (M+H)+=332; HPLC 1.44 tR=min.

The preparation of 2-chloro-4-(methylsulfonylmethyl)-6-morpholin-4-yl-pyrimidine was described earlier.

### EXAMPLE 13

### 3-Cyclopropyl-1-[4-[4-(1-cyclopropylsulfonylcyclopropyl)-6-morpholin-4-ylpyrimidin-2-yl]phenyl]urea

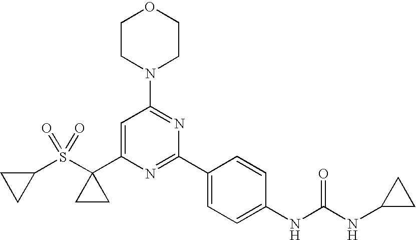

To a solution of phenyl N-[4-[4-(1-cyclopropylsulfonylcyclopropyl)-6-morpholin-4-ylpyrimidin-2-yl]phenyl]carbamate (100 mg, 0.19 mmol) in DMF (2 ML) was added triethylamine (0.08 mL, 0.58 mmol) followed by cyclopropylamine (55 mg, 0.96 mmol) and the reaction heated at 50° C. for 8 hours. The crude product was purified by preparative HPLC, using decreasingly polar mixtures of water (containing 1% NH3) and MeCN as eluents, to give the desired material as a white solid (60 mg).

NMR Spectrum: ¹H NMR (400.13 MHz, DMSO-d₆) δ 0.41-0.43 (2H, m), 0.64-0.66 (2H, m), 0.93-0.95 (2H, m), 1.02-1.05 (2H, m), 1.57 (2H, d), 1.65 (2H, d), 2.56 (1H, m), 3.02 (1H, s), 3.71 (8H, s), 6.42 (1H, s), 6.88 (1H, s), 7.50 (2H, d), 8.22 (2H, d), 8.53 (1H, s)

LCMS Spectrum: m/z (ESI+)(M+H)+=484; HPLC tR=1.80 min.

mTOR Kinase Assay (Echo): 0.00357 μM

The following compounds were made in an analogous fashion from either phenyl N-[4-[4-(1-cyclopropylsulfonylcyclopropyl)-6-morpholin-4-ylpyrimidin-2-yl]phenyl]carbamate, phenyl N-[4-[4-(1-cyclopropylsulfonylcyclopentyl)-6-morpholin-4-ylpyrimidin-2-yl]phenyl]carbamate or phenyl N-[4-[4-(1-cyclopropylsulfonylcyclobutyl)-6-morpholin-4-ylpyrimidin-2-yl]phenyl]carbamate and the appropriate amine.

| | | | LCMS | Retention |
|---|---|---|---|---|
| Example | Structure | NAME | MH+ | time (min) |
| 13a | 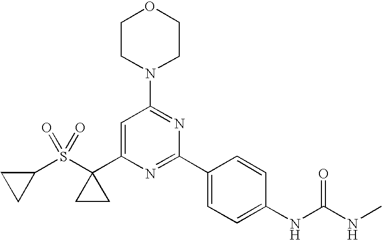 | 1-[4-[4-(1-cyclopropylsulfonylcyclopropyl)-6-morpholin-4-ylpyrimidin-2-yl]phenyl]-3-methylurea | 458.3 | 1.63 |
| 13b | 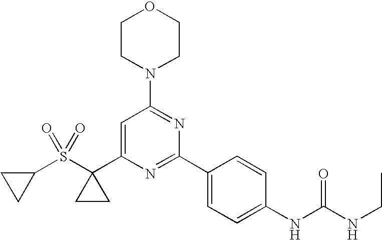 | 1-[4-[4-(1-cyclopropylsulfonylcyclopropyl)-6-morpholin-4-ylpyrimidin-2-yl]phenyl]-3-ethylurea | 472.4 | 1.78 |
| 13c | 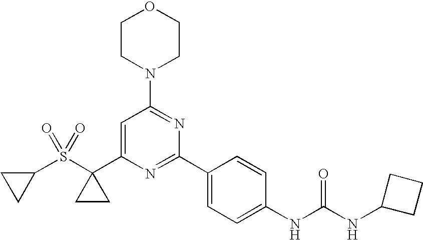 | 3-cyclobutyl-1-[4-[4-(1-cyclopropylsulfonylcyclopropyl)-6-morpholin-4-ylpyrimidin-2-yl]phenyl]urea | 498.4 | 2.04 |
| 13d | 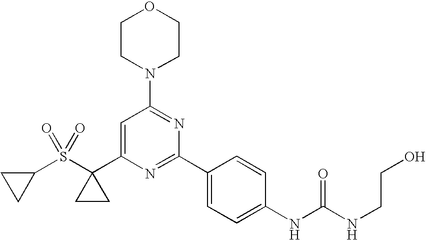 | 1-[4-[4-(1-cyclopropylsulfonylcyclopropyl)-6-morpholin-4-ylpyrimidin-2-yl]phenyl]-3-(2-hydroxyethyl)urea | 488.4 | 1.5 |
| 13e | 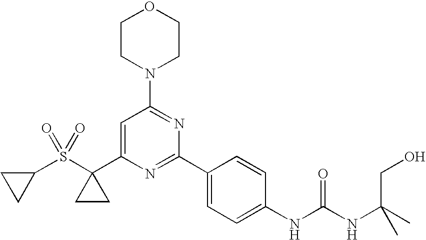 | 1-[4-[4-(1-cyclopropylsulfonylcyclopropyl)-6-morpholin-4-ylpyrimidin-2-yl]phenyl]-3-(1-hydroxy-2-methylpropan-2-yl)urea | 516.4 | 1.78 |
| 13f | 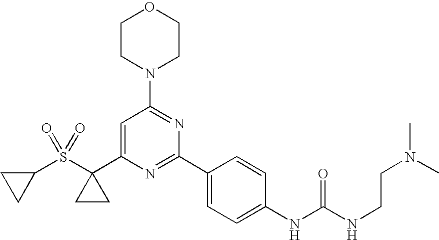 | 1-[4-[4-(1-cyclopropylsulfonylcyclopropyl)-6-morpholin-4-ylpyrimidin-2-yl]phenyl]-3-(2-dimethylaminoethyl)urea | 515.4 | 1.71 |
| 13g | 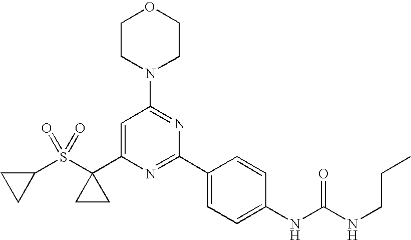 | 1-[4-[4-(1-cyclopropylsulfonylcyclopropyl)-6-morpholin-4-ylpyrimidin-2-yl]phenyl]-3-propylurea | 486.4 | 1.96 |
| 13h | 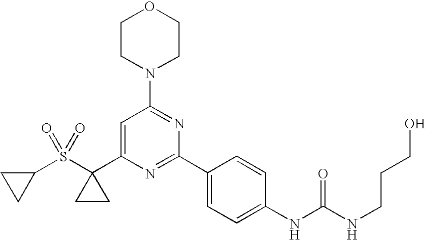 | 1-[4-[4-(1-cyclopropylsulfonylcyclopropyl)-6-morpholin-4-ylpyrimidin-2-yl]phenyl]-3-(3-hydroxypropyl)urea | 502.4 | 1.54 |
| 13i | 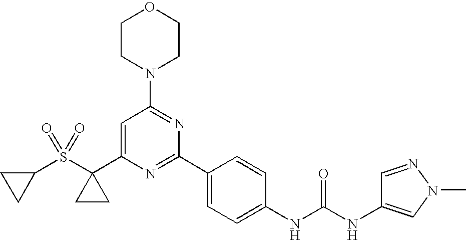 | 1-[4-[4-(1-cyclopropylsulfonylcyclopropyl)-6-morpholin-4-ylpyrimidin-2-yl]phenyl]-3-(1-methylpyrazol-4-yl)urea | 524.4 | 1.71 |
| 13j* | 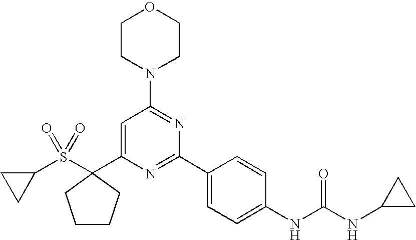 | 3-cyclopropyl-1-[4-[4-(1-cyclopropylsulfonylcyclopentyl)-6-morpholin-4-ylpyrimidin-2-yl]phenyl]urea | 512 | 2.30 |
| 13k* | 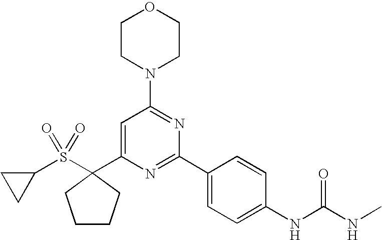 | 1-[4-[4-(1-cyclopropylsulfonylcyclopentyl)-6-morpholin-4-ylpyrimidin-2-yl]phenyl]-3-methylurea | 486 | 2.13 |
| 13l* | 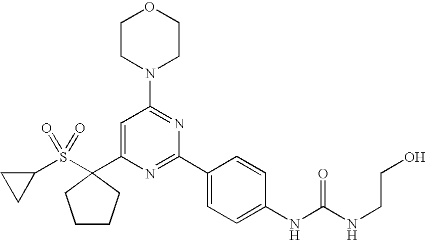 | 1-[4-[4-(1-cyclopropylsulfonylcyclopentyl)-6-morpholin-4-ylpyrimidin-2-yl]phenyl]-3-(2-hydroxyethyl)urea | 516 | 1.92 |
| 13m* | 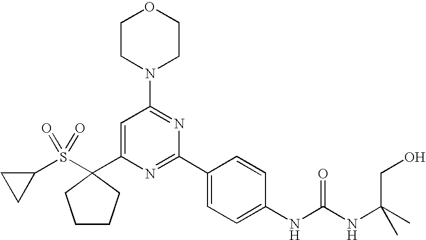 | 1-[4-[4-(1-cyclopropylsulfonylcyclopentyl)-6-morpholin-4-ylpyrimidin-2-yl]phenyl]-3-(1-hydroxy-2-methylpropan-2-yl)urea | 544 | 2.25 |
| 13n* | 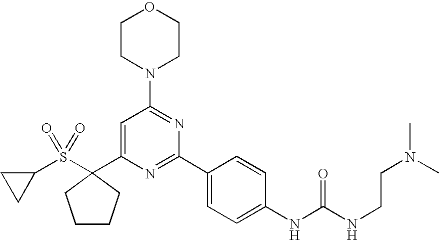 | 1-[4-[4-(1-cyclopropylsulfonylcyclopentyl)-6-morpholin-4-ylpyrimidin-2-yl]phenyl]-3-(2-dimethylaminoethyl)urea | 543 | 2.18 |
| 13o* | 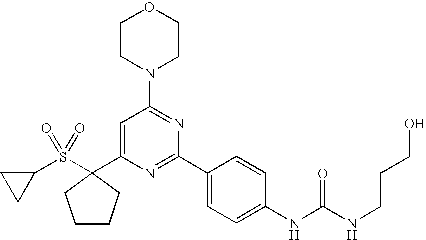 | 1-[4-[4-(1-cyclopropylsulfonylcyclopentyl)-6-morpholin-4-ylpyrimidin-2-yl]phenyl]-3-(3-hydroxypropyl)urea | 530 | 1.97 |
| 13p* | 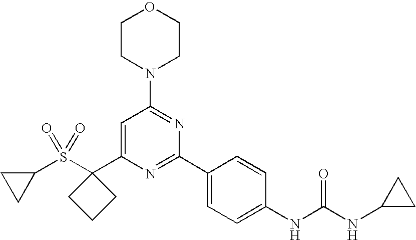 | 3-cyclopropyl-1-[4-[4-(1-cyclopropylsulfonylcyclobutyl)-6-morpholin-4-ylpyrimidin-2-yl]phenyl]urea | 498 | 2.05 |
| 13q* | 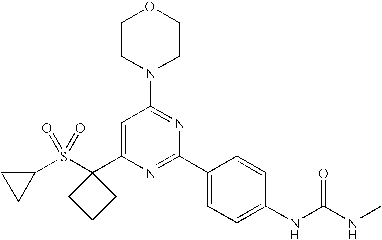 | 1-[4-[4-(1-cyclopropylsulfonylcyclobutyl)-6-morpholin-4-ylpyrimidin-2-yl]phenyl]-3-methylurea | 498 | 2.05 |
| 13r* | 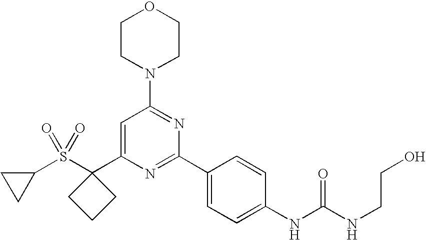 | 1-[4-[4-(1-cyclopropylsulfonylcyclobutyl)-6-morpholin-4-ylpyrimidin-2-yl]phenyl]-3-(2-hydroxyethyl)urea | 502 | 1.72 |
| 13s* | 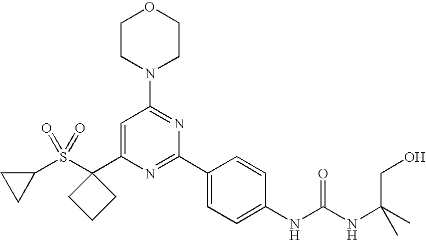 | 1-[4-[4-(1-cyclopropylsulfonylcyclobutyl)-6-morpholin-4-ylpyrimidin-2-yl]phenyl]-3-(1-hydroxy-2-methylpropan-2-yl)urea | 530 | 2.01 |
| 13t* | 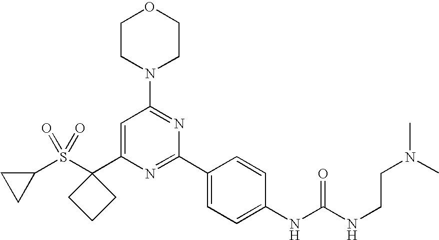 | 1-[4-[4-(1-cyclopropylsulfonylcyclobutyl)-6-morpholin-4-ylpyrimidin-2-yl]phenyl]-3-(2-dimethylaminoethyl)urea | 529 | 1.98 |
| 13u* | 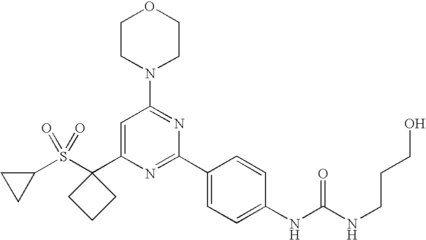 | 1-[4-[4-(1-cyclopropylsulfonylcyclobutyl)-6-morpholin-4-ylpyrimidin-2-yl]phenyl]-3-(3-hydroxypropyl)urea | 516 | 1.76 |

*Reaction stirred at 40° C. for 6 hours

### EXAMPLE 13a

¹H NMR (400.13 MHz, DMSO-d₆) δ 0.93-0.95 (2H, m), 1.02-1.05 (2H, m), 1.55-1.58 (2H, m), 1.63-1.66 (2H, m), 2.66 (3H, d), 3.02 (1H, s), 3.71 (8H, s), 6.06 (1H, d), 6.88 (1H, s), 7.48-7.51 (2H, m), 8.20-8.22 (2H, m), 8.73 (1H, s).

mTOR Kinase Assay (Echo): 0.00341 μM

### EXAMPLE 13b

¹H NMR (400.13 MHz, DMSO-d₆) δ 0.93-0.96 (2H, m), 1.02 (2H, m), 1.06 (3H, q), 1.56-1.58 (2H, m), 1.62-1.66 (2H, m), 3.00-3.04 (1H, m), 3.10-3.16 (2H, m), 3.71 (8H, s), 6.15 (1H, t), 6.88 (1H, s), 7.47-7.51 (2H, m), 8.20-8.23 (2H, m), 8.65 (1H, s).

mTOR Kinase Assay (Echo): 0.0037 μM

### EXAMPLE 13c

¹H NMR (400.13 MHz, DMSO-d₆) δ 0.93-1.00 (2H, m), 1.02-1.05 (2H, m), 1.56 (2H, d), 1.56-1.60 (2H, m), 1.61-1.68 (2H, m), 1.82-1.88 (2H, m), 2.17-2,24 (2H, m), 3.00-3.04 (1H, m), 3.71 (8H, s), 4.14 (1H, d), 6.45 (1H, d), 6.88 (1H, s), 7.46-7.48 (2H, m), 8.20-8.22 (2H, m), 8.55 (1H, s).

mTOR Kinase Assay (Echo): 0.0024 μM

### EXAMPLE 13d

¹H NMR (400.13 MHz, DMSO-d₆) δ 0.93-0.95 (2H, m), 1.03-1.04 (2H, d), 1.55-1.58 (2H, m), 1.63-1.66 (2H, m), 3.02 (1H, s), 3.18 (2H, d), 3.45 (2H, t), 3.71 (8H, s), 4.73 (1H, s), 6.25 (1H, s), 6.88 (1H, s), 7.47-7.49 (2H, m), 8.22 (2H, d), 8.80 (1H, s).

mTOR Kinase Assay (Echo): 0.00351 μM

### EXAMPLE 13e

¹H NMR (400.13 MHz, DMSO-d₆) δ 0.93-0.95 (2H, m), 1.03-1.05 (2H, m), 1.24 (6H, s), 1.56 (2H, d), 1.63-1.65 (2H, d), 3.02 (1H, m), 3.38-3.40 (2H,, m), 3.71 (8H, s), 4.95 (1H, s), 5.99 (1H, s), 6.88 (1H, s), 7.45 (2H, d), 8.20 (2H, d), 8.73 (1H, s).

mTOR Kinase Assay (Echo): 0.0301 μM

### EXAMPLE 13f

¹H NMR (400.13 MHz, DMSO-d₆) δ 0.93-0.96 (2H, m), 1.03-1.05 (2H, m), 1.55-1.58 (2H, m), 1.63-1.66 (2H, m), 2.18 (6H, s), 2.34 (2H, t), 3.02 (1H, t), 3.19 (2H, q), 3.71 (8H, s), 6.15 (1H, s), 6.88 (1H, s), 7.47-7.49 (2H, m), 8.20-8.22 (2H, m), 8.88 (1H, s).

mTOR Kinase Assay (Echo): 0.386 μM

### EXAMPLE 13g

¹H NMR (400.13 MHz, DMSO-d₆) δ 0.89 (3H, t), 0.93-0.96 (2H, m), 1.03 (2H, d), 1.41-1.50 (2H, m), 1.56-1.58 (2H, t), 1.62-1.66 (2H, m), 3.00-3.02 (1H, m), 3.06 (2H, s), 3.71 (8H, s), 6.19 (1H, t), 6.88 (1H, s), 7.49 (2H, d), 8.21 (2H, d), 8.64 (1H, s).

mTOR Kinase Assay (Echo): 0.00479 μM

### EXAMPLE 13h

¹H NMR (400.13 MHz, DMSO-d₆) δ 0.92-0.98 (2H, m), 0.98-1.07 (2H, m), 1.55-1.56 (2H, m), 1.58 (2H, d), 1.61-1.66 (2H, m), 2.99-3.05 (1H, m), 3.15-3.19 (2H, m), 3.45-3.50 (2H, m), 3.71 (8H, s), 4.47 (1H, t), 6.19 (1H, t), 6.88 (1H, s), 7.47-7.51 (2H, m), 8.20-8.23 (2H, m), 8.70 (1H, s).

mTOR Kinase Assay (Echo): 0.0106 μM

### EXAMPLE 13i

¹H NMR (400.13 MHz, DMSO-d₆) δ 0.94 (2H, t), 1.03-1.06 (2H, m), 1.55-1.59 (2H, m), 1.64-1.67 (2H, m), 3.02 (1H, s), 3.72 (8H, s), 3.79 (3H, s), 7.38 (1H, s), 7.54 (2H, d), 7.76 (1H, s), 8.25 (2H, d), 8.38 (1H, s), 8.84 (1H, s).

mTOR Kinase Assay (Echo): 0.00275 μM

### EXAMPLE 13j

¹H NMR (400.13 MHz, DMSO-d₆) δ 0.39-0.43 (2H, m), 0.62-0.67 (2H, m), 0.71-0.73 (2H, m), 0.84-0.87 (2H, m), 1.55 (2H, t), 1.81 (2H, d), 2.33 (1H, t), 2.43-2.45 (2H, m), 2.57-2.61 (1H, m), 2.82 (2H, t), 3.72 (8H, s), 6.45 (1H, d), 6.87 (1H, s), 7.50 (2H, d), 8.25 (2H, d), 8.56 (1H, s).

mTOR Kinase Assay (Echo): 0.0178 μM

### EXAMPLE 13k

¹H NMR (400.13 MHz, DMSO-d₆) δ 0.71-0.73 (2H, m), 0.84-0.87 (2H, m), 1.55 (2H, t), 1.79 (1H, d), 1.82 (1H, s), 2.43 (2H, t), 2.57-2.61 (1H, m), 2.65 (3H, d), 2.82 (2H, d), 3.72 (8H, s), 6.08 (1H, d), 6.86 (1H, s), 7.49-7.51 (2H, m), 8.23-8.25 (2H, m), 8.77 (1H, s).

mTOR Kinase Assay (Echo): 0.0179 μM

### EXAMPLE 13l

¹H NMR (400.13 MHz, DMSO-d₆) δ 0.71-0.73 (2H, m), 0.83-0.88 (2H, m), 1.55 (2H, t), 1.77-1.82 (2H, m), 2.43-2.48 (2H, m), 2.55-2.62 (1H, m), 2.81 (1H, t), 2.84 (1H, s), 3.15-3.18 (2H, m), 3.45 (2H, q), 3.72 (8H, s), 4.77 (1H, t), 6.26 (1H, t), 6.86 (1H, s), 7.47-7.50 (2H, m), 8.23-8.26 (2H, m), 8.83 (1H, s).

mTOR Kinase Assay (Echo): 0.0108 μM

### EXAMPLE 13m

¹H NMR (400.13 MHz, DMSO-d₆) δ 0.72-0.74 (2H, m), 0.85-0.88 (2H, m), 1.24 (6H, s), 1.55 (2H, t), 1.79 (2H, d), 2.42 (1H, s), 2.45 (1H, d), 2.59 (1H, s), 2.80 (2H, s), 3.38 (2H, d), 3.72 (8H, s), 4.99 (1H, t), 6.01 (1H, s), 6.86 (1H, s), 7.44-7.46 (2H, m), 8.22-8.24 (2H, m), 8.75 (1H, s).

mTOR Kinase Assay (Echo): 0.0532 μM

### EXAMPLE 13n

¹H NMR (400.13 MHz, DMSO-d₆) δ 0.71-0.73 (2H, m), 0.85-0.87 (2H, m), 1.55 (2H, t), 1.79 (2H, d), 2.17 (6H, s), 2.33 (2H, t), 2.42 (1H, s), 2.45 (1H, d), 2.59 (1H, s), 2.81 (2H, s), 3.19 (2H, q), 3.72 (8H, s), 6.17 (1H, s), 6.86 (1H, s), 7.47-7.49 (2H, m), 8.23-8.25 (2H, m), 8.92 (1H, s).

mTOR Kinase Assay (Echo): 0.997 μM

### EXAMPLE 13o

¹H NMR (400.13 MHz, DMSO-d₆) δ 0.71-0.73 (2H, m), 0.83-0.88 (2H, m), 1.53-1.62 (4H, m), 1.79-1.82 (1H, m), 1.81 (1H, d), 2.44 (2H, d), 2.57-2.61 (1H, m), 2.81 (1H, t), 2.84 (1H, s), 3.14-3.18 (2H, m), 3.44-3.49 (2H, m), 3.72 (8H, s), 4.52 (1H, t), 6.21 (1H, t), 6.86 (1H, s), 7.48-7.50 (2H, m), 8.23-8.25 (2H, m), 8.73 (1H, s).

mTOR Kinase Assay (Echo): 0.0287 μM

### EXAMPLE 13p

¹H NMR (400.13 MHz, DMSO-d₆) δ 0.40-0.44 (2H, m), 0.62-0.67 (2H, m), 0.74-0.78 (2H, m), 0.85-0.87 (2H, m), 1.88-1.91 (1H, m), 2.07 (1H, t), 2.50 (1H, s), 2.53-2.58 (1H, m), 2.85-2.89 (2H, m), 2.94-2.99 (2H, m), 3.73 (8H, s), 6.42 (1H, d), 6.77 (1H, s), 7.48-7.52 (2H, m), 8.23-8.26 (2H, m), 8.53 (1H, s).

mTOR Kinase Assay (Echo): 0.00202 μM

### EXAMPLE 13q

¹H NMR (400.13 MHz, DMSO-d₆) δ 0.74-0.78 (2H, m), 0.84-0.89 (2H, m), 1.88-1.91 (1H, m), 2.07 (1H, t), 2.50 (1H, m), 2.66 (3H, d), 2.85-2.89 (2H, m), 2.94-2.99 (2H, m), 3.72 (8H, s), 6.06 (1H, q), 6.76 (1H, s), 7.48-7.51 (2H, m), 8.22-8.26 (2H, m), 8.73 (1H, s).

mTOR Kinase Assay (Echo): 0.00192 μM

### EXAMPLE 13r

¹H NMR (400.13 MHz, DMSO-d₆) δ 0.76-0.78 (2H, m), 0.85-0.88 (2H, m), 1.91 (1H, s), 2.07 (1H, t), 2.50 (1H, s), 2.85-2.89 (2H, m), 2.94-2.97 (2H, m), 3.17 (2H, q), 3.46 (2H, q), 3.72 (8H, s), 4.72 (1H, t), 6.24 (1H, t), 6.76 (1H, s), 7.47-7.49 (2H, m), 8.23-8.25 (2H, m), 8.79 (1H, s).

mTOR Kinase Assay (Echo): 0.00198 μM

### EXAMPLE 13s

¹H NMR (400.13 MHz, DMSO-d₆) δ 0.76-0.79 (2H, m), 0.85-0.88 (2H, m), 1.24 (6H, s), 1.91 (1H, s), 2.07 (1H, t), 2.50 (1H, s), 2.85-2.89 (2H, m), 2.94 (1H, d), 2.96-2.97 (1H, m), 3.39 (2H, d), 3.72 (8H, s), 4.95 (1H, t), 6.00 (1H, s), 6.76 (1H, s), 7.44-7.46 (2H, m), 8.22-8.24 (2H, m), 8.73 (1H, s).

mTOR Kinase Assay (Echo): 0.00846 μM

### EXAMPLE 13t

¹H NMR (400.13 MHz, DMSO-d₆) δ 0.75-0.77 (2H, m), 0.85-0.88 (2H, m), 1.88-1.91 (1H, m), 2.07 (1H, t), 2.18 (6H, s), 2.32 (1H, s), 2.34 (2H, t), 2.86 (1H, d), 2.88 (1H, s), 2.94 (1H, d), 2.96 (1H, s), 3.20 (2H, t), 3.72 (8H, s), 6.15 (1H, s), 6.76 (1H, s), 7.47-7.49 (2H, m), 8.23-8.25 (2H, m), 8.88 (1H, s).

mTOR Kinase Assay (Echo): 0.172 μM

### EXAMPLE 13u

¹H NMR (400.13 MHz, DMSO-d₆) δ 0.74-0.78 (2H, m), 0.84-0.89 (2H, m), 1.57 (1H, d), 1.61 (1H, t), 1.88-1.91 (1H, m), 2.06 (1H, d), 2.50 (1H, m), 2.85-2.89 (2H, m), 2.94-2.99 (2H, m), 3.14-3.19 (2H, m), 3.45-3.49 (2H, m), 3.72 (8H, s), 4.47 (1H, t), 6.19 (1H, t), 6.76 (1H, s), 7.47-7.50 (2H, m), 8.23-8.25 (2H, m), 8.70 (1H, s).

mTOR Kinase Assay (Echo): 0.00477 μM

The preparation of phenyl N-[4-[4-(1-cyclopropylsulfonylcyclopropyl)-6-morpholin-4-ylpyrimidin-2-yl]phenyl]carbamate is described below:

### Phenyl N-[4-[4-(1-cyclopropylsulfonylcyclopropyl)-6-morpholin-4-ylpyrimidin-2-yl]phenyl]carbamate

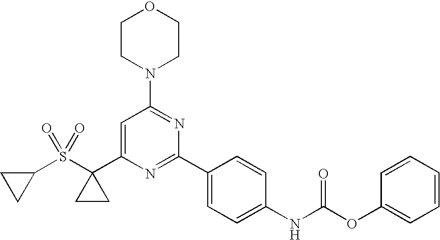

Phenyl chloroformate (0.440 mL, 3.50 mmol) was added dropwise to 4-[4-(1-cyclopropylsulfonylcyclopropyl)-6-morpholin-4-ylpyrimidin-2-yl]aniline (1.40g, 3.50 mmol) and sodium bicarbonate (0.440 g, 5.24 mmol) in dioxane (50 mL) under nitrogen. The resulting suspension was stirred at RT for 2 hours. The reaction mixture was evaporated to dryness, redissolved in ethyl acetate (100 mL) and washed with water (100 mL). The organic layer was dried (MgSO₄), filtered and evaporated to afford the desired material as a yellow solid (1.82 g).

NMR Spectrum: ¹H NMR (400.13 MHz, DMSO-d₆) δ 0.93-0.95 (2H, m), 1.05 (2H, d), 1.59 (1H, s), 1.60 (1H, t), 1.65 (1H, t), 1.67 (1H, d), 3.01-3.05 (1H, m), 3.58 (1H, s), 3.73 (8H, s), 6.94 (1H, s), 7.24-7.30 (3H, m), 7.43-7.47 (2H, m), 7.64 (2H, d), 8.31 (2H, d), 10.44 (1H, s)

LCMS Spectrum: m/z (ESI+) (M+H)+=521; HPLC tR=2.68 min.

### 4-[4-(1-Cyclopropylsulfonylcyclopropyl)-6-morpholin-4-ylpyrimidin-2-yl]aniline

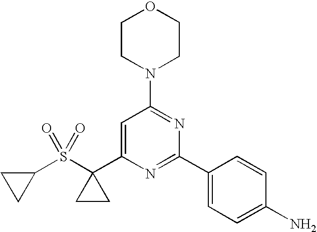

Dichlorobis(triphenylphosphine)palladium(II) (0.376 g, 0.54 mmol) was added to 2-chloro-4-(1-cyclopropylsulfonylcyclopropyl)-6-morpholin-4-ylpyrimidine (1.84 g, 5.35 mmol), 4-(4,4,5,5-tetramethyl-1,3,2-dioxaborolan-2-yl)aniline (1.466 g, 6.69 mmol) and sodium carbonate (13.38 mL, 26.76 mmol) in 18% DMF in 7:3:2 DME:Water:Ethanol (40 mL). The resulting solution was stirred at 85° C. for 1 hour. The reaction mixture was concentrated and diluted with DCM (200 mL), and washed with water (200 mL). The organic layer was dried (MgSO₄), filtered and evaporated to afford crude product. The crude product was purified by flash silica chromatography, eluting with 0 to 50% ethyl acetate in isohexane, to give the desired material as a yellow solid (1.40 g).

NMR Spectrum: ¹H NMR (400.13 MHz, DMSO-d₆) δ 0.91-0.97 (2H, m), 0.98-1.06 (2H, m), 1.52-1.58 (2H, m), 1.59-1.64 (2H, m), 2.97-3.04 (1H, m), 3.68 (4H, d), 3.71-3.71 (4H, m), 5.54 (2H, d), 6.58-6.62 (2H, m), 6.79 (1H, s), 8.03 (2H, d)

LCMS Spectrum: m/z (ESI+) (M+H)+=401; HPLC tR=1.62 min.

### 2-Chloro-4-(1-cyclopropylsulfonylcyclopropyl)-6-morpholin-4-ylpyrimidine

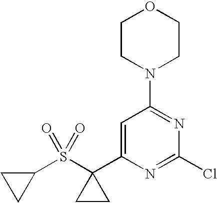

Sodium hydroxide (8.81 mL, 88.11 mmol) was added to 2-chloro-4-(cyclopropylsulfonylmethyl)-6-morpholin-4-ylpyrimidine (2.80 g, 8.81 mmol), 1,2-dibromoethane (1.519 mL, 17.62 mmol) and tetrabutylammonium bromide (0.568 g, 1.76 mmol) in toluene (120 mL) at RT. The resulting solution was stirred at 60° C. for 3 hours. The reaction mixture was evaporated to dryness, redissolved in ethyl acetate (200 mL), and washed sequentially with water (200 mL) and saturated brine (100 mL). The organic layer was dried (MgSO₄), filtered and evaporated to afford crude product. The crude product was purified by flash silica chromatography, eluting with 0 to 50% ethyl acetate in isohexane, to give the desired material as a yellow solid (1.84 g).

NMR Spectrum: ¹H NMR (400.13 MHz, DMSO-d₆) δ 0.89-0.93 (2H, m), 1.00-1.05 (2H, m),), 1.50 (2H, d), 1.62 (2H, d), 2.89-2.96 (1H, m), 3.65 (4H, m), 3.66-3.67 (4H, m), 7.01 (1H, s)

LCMS Spectrum: m/z (ESI+) (M+H)+=344; HPLC 1.48 tR=min.

### 2-Chloro-4-(cyclopropylsulfonylmethyl)-6-morpholin-4-ylpyrimidine

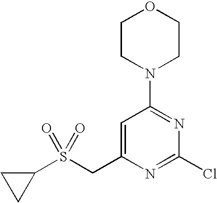

Cyclopropanesulfinic acid sodium salt (5.66 g, 44.17 mmol) was added in one portion to 2-chloro-4-(iodomethyl)-6-morpholin-4-ylpyrimidine (12.5 g, 36.81 mmol) in acetonitrile (300 mL) at RT. The resulting suspension was stirred at 80 ° C. for 24 hours. The resulting mixture was evaporated to dryness and the residue was azeotroped with MeCN to afford the desired material (7.12 g).

NMR Spectrum: ¹H NMR (400.13 MHz, DMSO-d₆) δ 0.94-0.99 (2H, m), 1.01-1.07 (2H, m), 2.77-2.84 (1H, m), 3.62 (4H, s), 3.67-3.69 (4H, s), 4.47 (2H, s), 6.95 (1H, s)

LCMS Spectrum: m/z (ESI+) (M+H)+=318; HPLC tR=1.46 min.

### 2-Chloro-4-(iodomethyl)-6-morpholin-4-ylpyrimidine

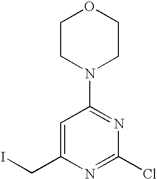

Sodium iodide (27.2 g, 181.31 mmol) was added to (2-chloro-6-morpholin-4-ylpyrimidin-4-yl)methyl methanesulfonate (27.9 g, 90.66 mmol) in acetone (400 mL) at RT under nitrogen. The resulting suspension was stirred at RT for 2 hours. The reaction mixture was evaporated to dryness, redissolved in DCM (400 mL) and was washed with water (200 mL). The organic layer was dried (MgSO₄), filtered and evaporated to afford the desired material as a brown solid (33.9 g).

NMR Spectrum: ¹H NMR (400.13 MHz, DMSO-d₆) δ 3.59 (4H, s), 3.63-3.68 (4H, m), 4.29 (2H, s), 6.97 (1H, s)

LCMS Spectrum: m/z (ESI+) (M+H)+339=; HPLC tR=1.87 min.

### (2-Chloro-6-morpholin-4-ylpyrimidin-4-yl)methyl methanesulfonate

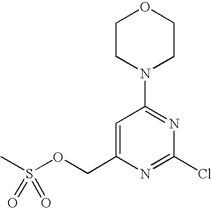

Methanesulfonyl chloride (10.57 mL, 136 mmol) was added dropwise to (2-chloro-6-morpholin-4-ylpyrimidin-4-yl)methanol (20.83 g, 90.70 mmol) and DIPEA (23.70 mL, 136 mmol) in DCM (375 mL) at 0° C. over a period of 15 minutes, under a nitrogen atmosphere. The resulting solution was stirred at RT for 1 hour. The reaction mixture was diluted with water (100 mL) and the organic layer dried (MgSO₄), filtered and evaporated to afford the desired material as a brown oil (27.9 g).

LCMS Spectrum: m/z (ESI+) (M+H)+=308; HPLC tR=1.58 min.

### (2-Chloro-6-morpholin-4-ylpyrimidin-4-yl)methanol

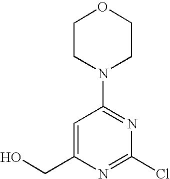

To a suspension of methyl 2-chloro-6-morpholin-4-ylpyrimidine-4-carboxylate (60 g, 232.85 mmol) in THF (1200 mL) at -5° C. was added lithium borohydride (2M in THF, 0. 122 L, 244.50 mmol) dropwise over 30 minutes. The reaction mixture was warmed to RT and stirred for 1 hour. To this was added water (600 mL), the mixture stirred for 2 hours and then filtered. Further water (600 mL) was added and the solution was extracted three times with ethyl acetate (600 mL). The combined organics were washed with 50% aqueous brine (900 mL), dried (MgSO₄) and the solvent was then removed under reduced pressure to give the desired product as a white solid (49.8 g).

NMR Spectrum: ¹H NMR (400.13 MHz, DMSO-d₆) δ 3.59-3.68 (8H, m), 4.35 (2H, dd), 5.50 (1H, t), 6.77 (1H, s).

LCMS Spectrum: m/z (ESI+) (M+H)+=230; HPLC tR=1.08 min

### Methyl 2-chloro-6-morpholin-4-ylpyrimidine-4-carboxylate

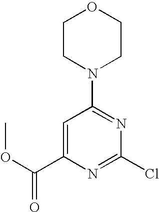

To a solution of 2,6-dichloropyrimidine-4-carboxylate (60 g, 289.84 mmol) in DCM (400 mL) at −5° C. was added triethylamine (44.4 mL, 318.82 mmol), washing in with DCM (80 mL). To the resulting solution was added morpholine (26.6 mL, 304.33 mmol) in DCM (120 mL) dropwise over 2 hours, maintaining the temperature below 5° C. The reaction mixture was stirred at 0° C. for 2 hours and then warmed to RT. Water (600 mL) was added and the layers were separated. The organic layer was washed twice with water (180 mL) and the combined aqueous fractions extracted twice with DCM (180 mL). The combined organics were washed twice with 75% aqueous brine (180 mL), dried (MgSO₄) and the solvent removed under reduced pressure to give the crude product. This was purified by crystallisation from ethyl acetate/isohexane to give the desired product as a white solid (65.4g).

NMR Spectrum: ¹H NMR (400.13 MHz, CDCl₃) δ 3.72-3.82 (8H, m), 3.99 (3H, s), 7.20 (1H, s).

LCMS Spectrum: m/z (ESI+) (M+H)+=258; HPLC tR=1.38 min

The preparation of phenyl N-[4-[4-( 1-cyclopropylsulfonylcyclopentyl)-6-morpholin-4-ylpyrimidin-2-yl]phenyl]carbamate is described below.

### Phenyl N-[4-[4-(1-cyclopropylsulfonylcyclopentyl)-6-morpholin-4-ylpyrimidin-2-yl]phenyl]carbamate

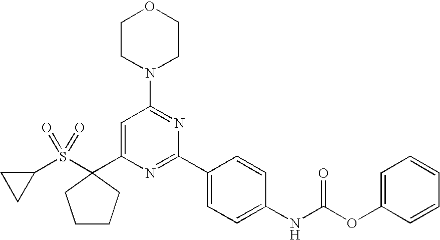

Phenyl chloroformate (0.455 mL, 3.62 mmol) was added dropwise to 4-[4-(1-cyclopropylsulfonylcyclopentyl)-6-morpholin-4-ylpyrimidin-2-yl]aniline (1.55 g, 3.62 mmol) and sodium bicarbonate (0.456 g, 5.43 mmol) in dioxane (50 mL) under nitrogen. The resulting suspension was stirred at RT for 3 hours. The reaction mixture was evaporated to dryness, redissolved in ethyl acetate (100 mL) and washed with water (100 mL). The organic layer was dried (MgSO₄), filtered and evaporated to give the desired material as a yellow solid (2.31 g).

NMR Spectrum: ¹H NMR (400.13 MHz, DMSO-d₆) δ 0.71-0.75 (2H, m), 0.84-0.89 (2H, m), 1.56 (2H, t), 1.78-1.83 (2H, m), 2.45 (1H, t), 2.57-2.63 (2H, m), 2.81-2.84 (2H, m), 3.73 (8H, s), 6.90 (1H, s), 7.23-7.30 (3H, m), 7.43-7.47 (2H, m), 7.63 (2H, d), 8.33-8.36 (2H, m), 10.42 (1H, s)

LCMS Spectrum: m/z (ESI+) (M+H)+=549; HPLC tR=3.02 min.

### 4-[4-(1-Cyclopropylsulfonylcyclopentyl)-6-morpholin-4-ylpyrimidin-2-yl]aniline

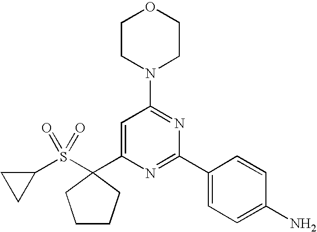

Dichlorobis(triphenylphosphine)palladium(II) (0.170 g, 0.24 mmol) was added to a degassed solution of 2-chloro-4-(1-cyclopropylsulfonylcyclopentyl)-6-morpholin-4-ylpyrimidine (1.80 g, 4.84 mmol), 4-(4,4,5,5-tetramethyl-1,3,2-dioxaborolan-2-yl)aniline (1.326 g, 6.05 mmol) and an aqueous solution of sodium carbonate (12.10 mL, 24.20 mmol) in a solvent mixture of 18% DMF in 7:3:2 DME:Water:Ethanol (40 mL). The resulting solution was stirred at 85° C. for 1 hour. The reaction mixture was concentrated, diluted with DCM (100 mL), and washed with water (100 mL). The organic layer was dried (MgSO₄), filtered and evaporated to afford crude product. The crude product was purified by flash silica chromatography, elution gradient 0 to 70% ethyl acetate in isohexane, to give the desired material as a yellow solid (1.55 g).

NMR Spectrum: ¹H NMR (400.13 MHz, DMSO-d₆) δ 0.73-0.75 (2H, m), 0.83-0.88 (2H, m), 1.55 (2H, t), 1.78 (1H, s), 1.81 (1H, t), 2.41 (1H, d), 2.45-2.47 (1H, m), 2.54-2.58 (1H, m), 2.79 (1H, t), 2.82 (1H, s), 3.70 (8H, d), 5.53 (2H, s), 6.59-6.62 (2H, m), 6.77 (1H, s), 8.07-8.09 (2H, m)

LCMS Spectrum: m/z (ESI+) (M+H)+=429; HPLC tR=2.36 min.

### 2-Chloro-4-(1-cyclopropylsulfonylcyclopentyl)-6-morpholin-4-ylpyrimidine

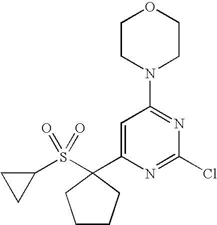

10N Sodium hydroxide solution (5.70 mL, 56.96 mmol) was added to 2-chloro-4-(cyclopropylsulfonylmethyl)-6-morpholin-4-ylpyrimidine (1.81 g, 5.70 mmol), 1,4-dibromobutane (0.674 mL, 5.70 mmol) and tetrabutylammonium bromide (0.367 g, 1.14 mmol) in toluene (100 mL) at RT. The resulting solution was stirred at 60° C. for 3 hours. The reaction mixture was evaporated to dryness, redissolved in ethyl acetate (75 mL) and washed with water (75 mL). The organic layer was dried (MgSO₄), filtered and evaporated to afford crude product. The crude product was purified by flash silica chromatography, elution gradient 0 to 40% ethyl acetate in DCM, to give the desired material as a white solid (1.8 g).

NMR Spectrum: ¹H NMR (400.13 MHz, DMSO-d₆) δ 0.71-0.75 (2H, m), 0.90-0.94 (2H, m), 1.50-1.59 (2H, m), 1.77-1.80 (2H, m), 2.36-2.44 (2H, m), 2.57-2.68 (3H, m_), 3.67 (8H, s), 6.97 (1H, s)

LCMS Spectrum: m/z (ESI+) (M+H)+=372; HPLC tR=2.26 min.

The preparation of 2-chloro-4-(cyclopropylsulfonylmethyl)-6-morpholin-4-ylpyrimidine was described earlier.

The preparation of phenyl N-[4-[4-(1-cyclopropylsulfonylcyclobutyl)-6-morpholin-4-ylpyrimidin-2-yl]phenyl]carbamate is described below.

### Phenyl N-[4-[4-(1-cyclopropylsulfonylcyclobutyl)-6-morpholin-4-ylpyrimidin-2-yl]phenyl]carbamate

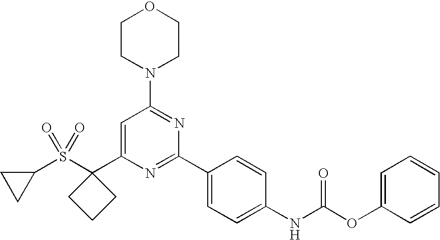

Phenyl chloroformate (0.106 mL, 0.84 mmol) was added dropwise to 4-[4-(1-cyclopropylsulfonylcyclobutyl)-6-morpholin-4-ylpyrimidin-2-yl]aniline (350 mg, 0.84 mmol) and sodium bicarbonate (106 mg, 1.27 mmol) in dioxane (20 mL) under nitrogen. The resulting suspension was stirred at RT for 2 hours. The reaction mixture was evaporated to dryness, redissolved in ethyl acetate (50 mL) and washed with water (50 mL). The organic layer was dried (MgSO₄), filtered and evaporated to give the desired material as a cream solid (453 mg).

NMR Spectrum: ¹H NMR (400.13 MHz, DMSO-d₆) δ 0.75-0.77 (2H, m), 0.85-0.88 (2H, m), 1.90 (1H, d), 2.07 (1H, d), 2.54(1H, m), 2.86-2.90 (2H, m), 2.93-2.98 (2H, s), 3.73 (8H, s), 6.80 (1H, s), 7.24-7.30 (3H, m), 7.43-7.47 (2H, m), 7.63 (2H, d), 8.33-8.35 (2H, m), 10.42 (1H, s)

LCMS Spectrum: m/z (ESI+) (M+H)+=535; HPLC tR=2.91 min.

### 4-[4-(1-Cyclopropylsulfonylcyclobutyl)-6-morpholin-4-ylpyrimidin-2-yl]aniline

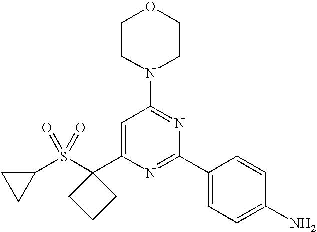

2-Chloro-4-(1-cyclopropylsulfonylcyclobutyl)-6-morpholin-4-ylpyrimidine (430 mg, 1.20 mmol), 4-(4,4,5,5-tetramethyl-1,3,2-dioxaborolan-2-yl)aniline (329 mg, 1.50 mmol), dichlorobis(triphenylphosphine)palladium(II) (42.2 mg, 0.06 mmol) and an aqueous solution of sodium carbonate (3.00 mL, 6.01 mmol) were suspended in a solvent mixture of 18% DMF in 7:3:2 DME:Water:Ethanol (8 mL) and sealed into a microwave tube. The reaction was heated to 100° C. for 30 minutes in the microwave reactor and cooled to RT. The crude product was purified by ion exchange chromatography using an SCX column, the desired product was eluted from the column using 7M ammonia in methanol. The isolated material was further purified by flash silica chromatography, elution gradient 0 to 50% ethyl acetate in DCM, to give the desired material as a white solid (350 mg).

NMR Spectrum: ¹H NMR (400.13 MHz, DMSO-d₆) δ 0.74-0.78 (2H, m), 0.83-0.88 (2H, m), 1.85-1.91 (1H, m), 2.03-2.07 (1H, m), 2.44-2.48 (1H, m), 2.83-2.87 (2H, m), 2.90-2.97 (2H, m), 3.70-3.71 (8H, m), 5.52 (2H, d), 6.58-6.62 (2H, m), 6.67 (1H, s), 8.06-8.09 (2H, m)

LCMS Spectrum: m/z (ESI+) (M+H)+=415; HPLC tR=2.19 min.

### 2-Chloro-4-(1-cyclopropylsulfonylcyclobutyl)-6-morpholin-4-ylpyrimidine

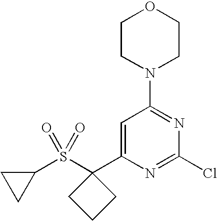

10N Sodium hydroxide solution (9.60 mL, 95.97 mmol) was added to 1,3-dibromopropane (0.979 mL, 9.60 mmol), ²-chloro-⁴-(cyclopropylsulfonylmethyl)-6-morpholin-4-ylpyrimidine (2.80 g, 9.60 mmol) and tetrabutylammonium bromide (0.619 g, 1.92 mmol) in toluene (120 mL) at RT. The resulting solution was stirred at 60° C. for 18 hours. The reaction mixture was evaporated to dryness, redissolved in ethyl acetate (200 mL), and washed sequentially with water (200 mL) and saturated brine solution (100 mL). The organic layer was dried (MgSO₄), filtered and evaporated to afford crude product. The crude product was purified by flash silica chromatography, elution gradient 0 to 50% ethyl acetate in isohexane, to give the desired material as a yellow solid (0.795 g).

NMR Spectrum: ¹H NMR (400.13 MHz, DMSO-d₆) δ 0.73-0.77 (2H, m), 0.89-0.94 (2H, m), 1.85-1.92 (1H, m), 2.04-2.08 (1H, m), 2.53-2.57 (1H, m), 2.70-2.78 (2H, m), 2.85-2.93 (2H, m), 3.66 (8H, d), 6.88 (1H, s)

LCMS Spectrum: m/z (ESI+) (M+H)+=358; HPLC 1.95 tR=min.

The preparation of 2-chloro-4-(cyclopropylsulfonylmethyl)-6-morpholin-4-ylpyrimidine was described earlier.

### EXAMPLE 14

### 3-Cyclopropyl-1-[5-[4-(1-cyclopropylsulfonylcyclopropyl)-6-[(3S)-3-methylmorpholin-4-yl]pyrimidin-2-yl]pyridin-2-yl]urea

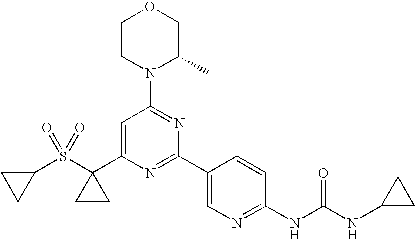

Triethylamine (0.208 mL, 1.5 mmol) was added to a solution of phenyl N-[5-[4-(1-cyclopropylsulfonylcyclopropyl)-6-[(3S)-3-methylmorpholin-4-yl]pyrimidin-2-yl]pyridin-2-yl]carbamate (200 mg, 0.37 mmol) and cyclopropylamine (1.48 mmol) in NMP (2 mL). The reaction was heated at 75° C. for 4 hours then purified by prep HPLC, using a mixture of water (containing 1% NH3) and acetonitrile as eluents, to give the desired material as a solid (40 mg).

NMR Spectrum: ¹H NMR (400.13 MHz, DMSO-d₆) δ 0.44-0.50 (2H, m), 0.64-0.72 (2H, m), 0.89-0.96 (2H, m), 1.00-1.06 (2H, m), 1.23 (3H, d), 1.54-1.60 (2H, m), 1.59-1.68 (2H, m), 2.59-2.66 (1H, m), 2.96-3.01 (1H, m), 3.16-3.25 (1H, m), 3.44-3.52 (1H, m), 3.63 (1H, dd), 3.76 (1H, d), 3.95-3.99 (1H, m), 4.20 (1H, s), 4.54 (1H, s), 6.90 (1H, s), 7.50 (1H, d), 8.18 (1H, s), 8.50 (1H, d), 9.09 (1H, s), 9.37 (1H, s)

LCMS Spectrum: m/z (ESI+)(M+H)+=499; HPLC tR=2.15 min.

mTOR Kinase Assay (Echo): 0.0465 μM

The following compounds were prepared in an analogous fashion from either phenyl N-[5-[4-(1-cyclopropylsulfonylcyclopropyl)-6-[(3S)-3-methylmorpholin-4-yl]pyrimidin-2-yl]pyridin-2-yl]carbamate, phenyl N-[5-[4-[1-(4-fluorophenyl)sulfonylcyclopropyl]-6-[(3S)-3-methylmorpholin-4-yl]pyrimidin-2-yl]pyrimidin-2-yl]carbamate, phenyl N-[5-[4-[1-(4-fluorophenyl)sulfonylcyclopropyl]-6-[(3S)-3-methylmorpholin-4-yl]pyrimidin-2-yl]pyridin-2-yl]carbamate, phenyl N-[5-[4-[(3S)-3-methylmorpholin-4-yl]-6-(1-methylsulfonylcyclopentyl)pyrimidin-2-yl]pyridin-2-yl]carbamate, phenyl N-[5-[4-[(3S)-3-methylmorpholin-4-yl]-6-(1-methylsulfonylcyclopentyl)pyrimidin-2-yl]pyrimidin-2-yl]carbamate or phenyl N-[5-[4-(1-cyclopropylsulfonylcyclopropyl)-6-[(3S)-3-methylmorpholin-4-yl]pyrimidin-2-yl]pyrimidin-2-yl]carbamate using the appropriate amine.

| | | | LCMS | Retention |
|---|---|---|---|---|
| Example | Structure | NAME | MH+ | time (min) |
| 14a | 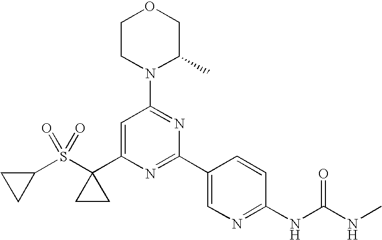 | 1-[5-[4-(1-cyclopropylsulfonylcyclopropyl)-6-[(3S)-3-methylmorpholin-4-yl]pyrimidin-2-yl]pyridin-2-yl]-3-methylurea | 473 | 2.03 |
| 14b | 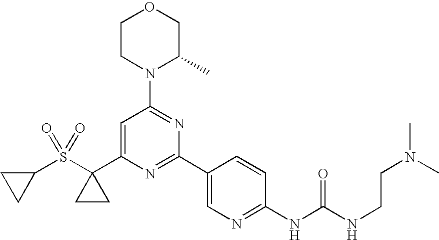 | 1-[5-[4-(1-cyclopropylsulfonylcyclopropyl)-6-[(3S)-3-methylmorpholin-4-yl]pyrimidin-2-yl]pyridin-2-yl]-3-(2-dimethylaminoethyl)urea | 530 | 1.97 |
| 14c | 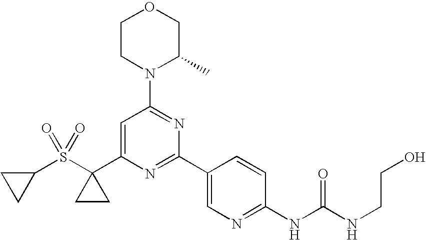 | 1-[5-[4-(1-cyclopropylsulfonylcyclopropyl)-6-[(3S)-3-methylmorpholin-4-yl]pyrimidin-2-yl]pyridin-2-yl]-3-(2-hydroxyethyl)urea | 503 | 1.73 |
| 14d | 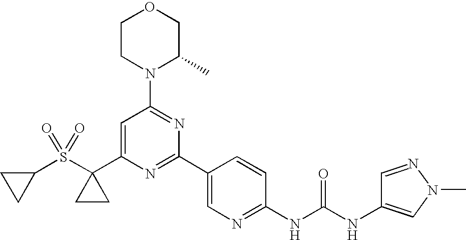 | 1-[5-[4-(1-cyclopropylsulfonylcyclopropyl)-6-[(3S)-3-methylmorpholin-4-yl]pyrimidin-2-yl]pyridin-2-yl]-3-(1-methylpyrazol-4-yl)urea | 539 | 2.01 |
| 14e | 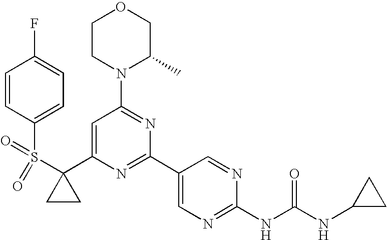 | 3-cyclopropyl-1-[5-[4-[1-(4-fluorophenyl)sulfonylcyclopro-pyl]-6-[(3S)-3-methylmorpholin-4-yl]pyrimidin-2-yl]pyrimidin-2-yl]urea | 554 | 2.37 |
| 14f | 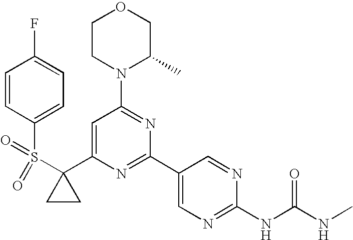 | 1-[5-[4-[1-(4-fluorophenyl)sulfonylcyclopro-pyl]-6-[(3S)-3-methylmorpholin-4-yl]pyrimidin-2-yl]pyrimidin-2-yl]-3-methylurea | 528 | 2.16 |
| 14g | 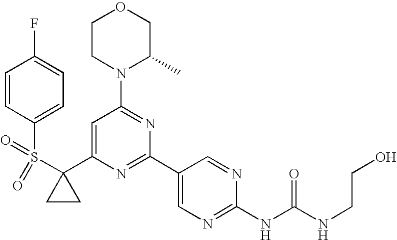 | 1-[5-[4-[1-(4-fluorophenyl)sulfonylcyclopro-pyl]-6-[(3S)-3-methylmorpholin-4-yl]pyrimidin-2-yl]pyrimidin-2-yl]-3-(2-hydroxyethyl)urea | 558 | 1.91 |
| 14h | 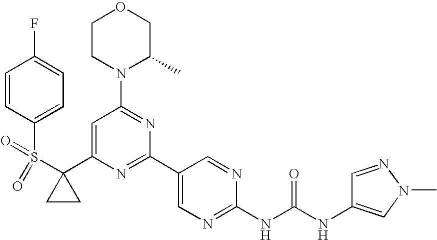 | 1-[5-[4-[1-(4-fluorophenyl)sulfonylcyclopro-pyl]-6-[(3S)-3-methylmorpholin-4-yl]pyrimidin-2-yl]pyrimidin-2-yl]-3-(1-methylpyrazol-4-yl)urea | 594 | 2.19 |
| 14i | 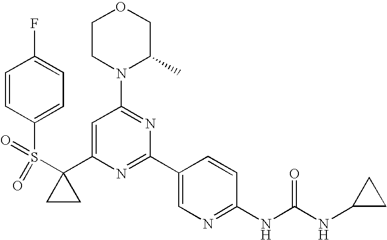 | 3-cyclopropyl-1-[5-[4-[1-(4-fluorophenyl)sulfonylcyclopro-pyl]-6-[(3S)-3-methylmorpholin-4-yl]pyrimidin-2-yl]pyridin-2-yl]urea | 553 | 2.26 |
| 14j | 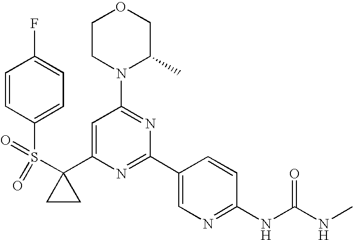 | 1-[5-[4-[1-(4-fluorophenyl)sulfonylcyclopro-pyl]-6-[(3S)-3-methylmorpholin-4-yl]pyrimidin-2-yl]pyridin-2-yl]-3-methylurea | 527 | 2.20 |
| 14k | 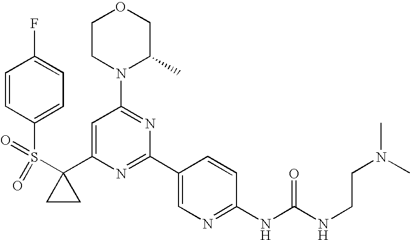 | 3-(2-dimethylaminoethyl)-1-[5-[4-[1-(4-fluorophenyl)sulfonylcyclopro-pyl]-6-[(3S)-3-methylmorpholin-4-yl]pyrimidin-2-yl]pyridin-2-yl]urea | 584 | 2.22 |
| 14l | 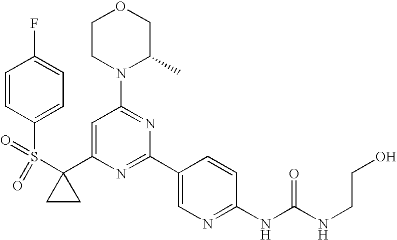 | 1-[5-[4-[1-(4-fluorophenyl)sulfonylcyclopro-pyl]-6-[(3S)-3-methylmorpholin-4-yl]pyrimidin-2-yl]pyridin-2-yl]-3-(2-hydroxyethyl)urea | 557 | 1.92 |
| 14m | 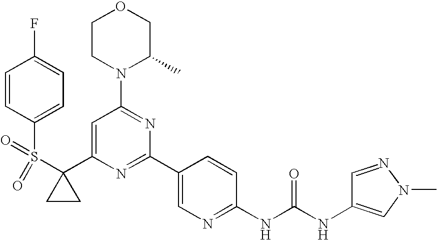 | 1-[5-[4-[1-(4-fluorophenyl)sulfonylcyclopro-pyl]-6-[(3S)-3-methylmorpholin-4-yl]pyrimidin-2-yl]pyridin-2-yl]-3-(1-methylpyrazol-4-yl)urea | 593 | 2.13 |
| 14n | 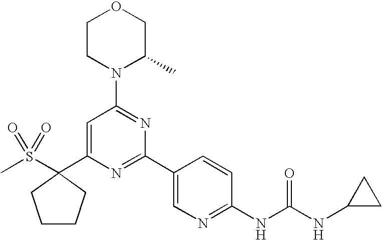 | 3-cyclopropyl-1-[5-[4-[(3S)-3-methylmorpholin-4-yl]-6-(1-methylsulfonylcyclopentyl)pyri-midin-2-yl]pyridin-2-yl]urea | 501 | 2.18 |
| 14o | 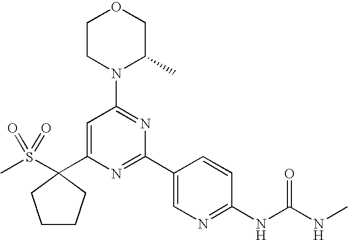 | 3-methyl-1-[5-[4-[(3S)-3-methylmorpholin-4-yl]-6-(1-methylsulfonylcyclopentyl)pyri-midin-2-yl]pyridin-2-yl]urea | 475 | 2.01 |
| 14p | 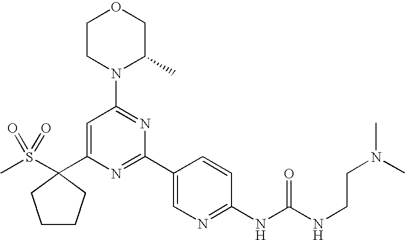 | 3-(2-dimethylaminoethyl)-1-[5-[4-[(3S)-3-methylmorpholin-4-yl]-6-(1-methylsulfonylcyclopentyl)pyri-midin-2-yl]pyridin-2-yl]urea | 532 | 2.01 |
| 14q | 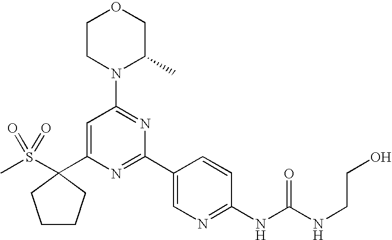 | 3-(2-hydroxyethyl)-1-[5-[4-[(3S)-3-methylmorpholin-4-yl]-6-(1-methylsulfonylcyclopentyl)pyri-midin-2-yl]pyridin-2-yl]urea | 505 | 1.78 |
| 14r | 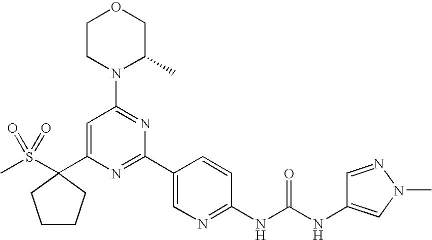 | 1-[5-[4-[(3S)-3-methylmorpholin-4-yl]-6-(1-methylsulfonylcyclopentyl)pyri-midin-2-yl]pyridin-2-yl]-3-(1-methylpyrazol-4-yl)urea | 541 | 2.01 |
| 14s | 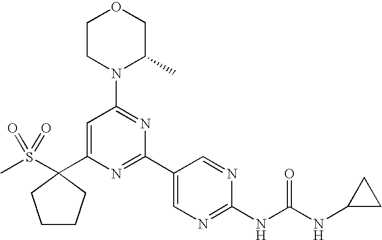 | 3-cyclopropyl-1-[5-[4-[(3S)-3-methylmorpholin-4-yl]-6-(1-methylsulfonylcyclopentyl)pyri-midin-2-yl]pyrimidin-2-yl]urea | 502 | 2.15 |
| 14t | 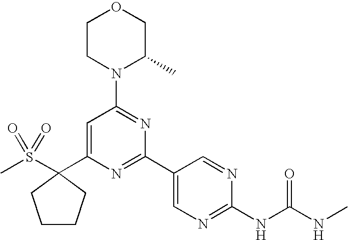 | 3-methyl-1-[5-[4-[(3S)-3-methylmorpholin-4-yl]-6-(1-methylsulfonylcyclopentyl)pyri-midin-2-yl]pyrimidin-2-yl]urea | 476 | 1.99 |
| 14u | 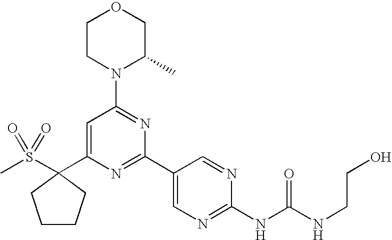 | 3-(2-hydroxyethyl)-1-[5-[4-[(3S)-3-methylmorpholin-4-yl]-6-(1-methylsulfonylcyclopentyl)pyri-midin-2-yl]pyrimidin-2-yl]urea | 506 | 1.70 |
| 14v | 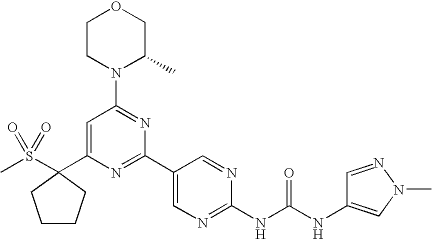 | 1-[5-[4-[(3S)-3-methylmorpholin-4-yl]-6-(1-methylsulfonylcyclopentyl)pyri-midin-2-yl]pyrimidin-2-yl]-3-(1-methylpyrazol-4-yl)urea | 542 | 1.95 |
| 14w | 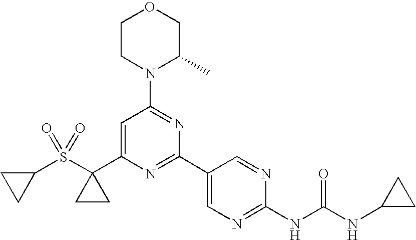 | 3-cyclopropyl-1-[5-[4-(1-cyclopropylsulfonylcyclopropyl)-6-[(3S)-3-methylmorpholin-4-yl]pyrimidin-2-yl]pyrimidin-2-yl]urea | 500 | 2.0 |
| 14x | 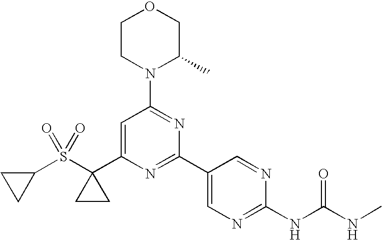 | 1-[5-[4-(1-cyclopropylsulfonylcyclopropyl)-6-[(3S)-3-methylmorpholin-4-yl]pyrimidin-2-yl]pyrimidin-2-yl]-3-methylurea | 474 | 1.79 |
| 14y | 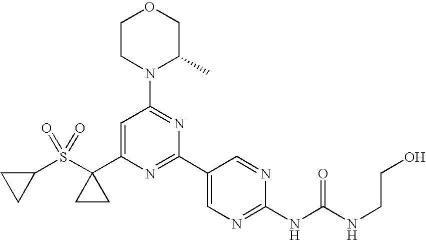 | 1-[5-[4-(1-cyclopropylsulfonylcyclopropyl)-6-[(3S)-3-methylmorpholin-4-yl]pyrimidin-2-yl]pyrimidin-2-yl]-3-(2-hydroxyethyl)urea | 504 | 1.54 |
| 14z | 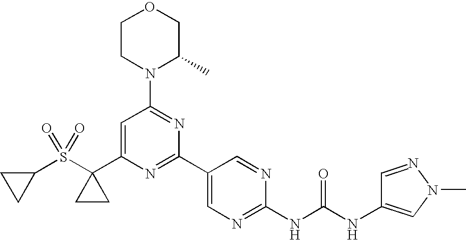 | 1-[5-[4-(1-cyclopropylsulfonylcyclopropyl)-6-[(3S)-3-methylmorpholin-4-yl]pyrimidin-2-yl]pyrimidin-2-yl]-3-(1-methylpyrazol-4-yl)urea | 540 | 1.82 |
| 14aa* | 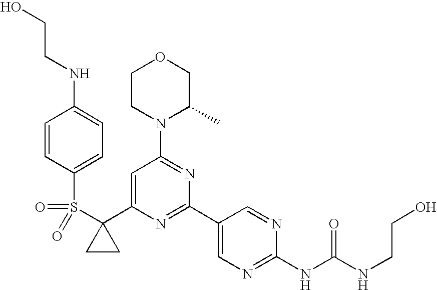 | 3-(2-hydroxyethyl)-1-[5-[4-[1-[4-(2-hydroxyethylamino)phenyl]sulf-onylcyclopropyl]-6-[(3S)-3-methylmorpholin-4-yl]pyrimidin-2-yl]pyrimidin-2-yl]urea | 599 | 1.63 |

*Isolated as a by product from the reaction to generate 14g

### EXAMPLE 14a

¹H NMR (400.13 MHz, DMSO-d₆) δ 0.90-0.96 (2H, m), 1.01-1.06 (2H, m), 1.24 (3H, d), 1.55-1.60 (2H, m), 1.62-1.68 (2H, m), 2.78 (3H, s), 3.18-3.27 (1H, m), 3.44-3.54 (1H, m), 3.64 (1H, d), 3.76 (1H, d), 3.97 (1H, d), 4.20 (1H, d), 4.56 (1H, s), 6.91 (1H, s), 7.43 (1H, d), 8.15 (1H, s), 8.49 (1H, d), 9.12 (1H, s), 9.48 (1H, s), 9.49 (1H, s).

mTOR Kinase Assay (Echo): 0.0136 μM

### EXAMPLE 14b

¹H NMR (400.13 MHz, DMSO-d₆) δ 0.91-0.95 (2H, m), 1.00-1.05 (2H, m), 1.23 (3H, d), 1.56-1.60 (2H, m), 1.63-1.66 (2H, m), 2.18 (6H, s), 2.36 (2H, t), 2.98-3.04 (1H, m), 3.17-3.29 (3H, m), 3.48 (1H, td), 3.63 (1H, dd), 3.76 (1H, d), 3.97 (1H, dd), 4.20 (1H, s), 4.54 (1H, s), 6.90 (1H, s), 7.53 (1H, d), 8.05 (1H, s), 8.49 (1H, d), 9.09 (1H, d), 9.48 (1H, s).

mTOR Kinase Assay (Echo): 1.39 μM

### EXAMPLE 14c

¹H NMR (400.13 MHz, DMSO-d₆) δ 0.90-0.94 (2H, m), 1.01-1.05 (2H, m), 1.23 (3H, d), 1.55-1.60 (2H, m), 1.63-1.67 (2H, m), 2.96-3.03 (1H, m), 3.16-3.28 (3H, m), 3.43-3.52 (2H, m), 3.63 (1H, dd), 3.76 (1H, d), 3.97 (1H, dd), 4.20 (1H, s), 4.54 (1H, s), 4.80 (1H, t), 6.90 (1H, s), 7.47 (1H, d), 8.29 (1H, s), 8.50 (1H, d), 9.10 (1H, s).

mTOR Kinase Assay (Echo): 0.0401 μM

### EXAMPLE 14d

¹H NMR (400.13 MHz, DMSO-d₆) δ 0.89-0.96 (2H, m), 1.00-1.05 (2H, m), 1.24 (3H, d), 1.58-1.61 (2H, m), 1.64-1.70 (2H, m), 2.97-3.04 (1H, m), 3.22 (1H, td), 3.43-3.55 (1H, m), 3.64 (1H, dd), 3.75-3.82 (4H, m), 3.98 (1H, dd), 4.23 (1H, s), 4.55 (1H, s), 6.92 (1H, s), 7.49 (1H, d), 7.87 (1H, s), 8.55 (1H, d), 9.18 (1H, s), 9.73 (1H, s), 10.36 (1H, s).

mTOR Kinase Assay (Echo): 0.0143 μM

### EXAMPLE 14e

¹H NMR (400.13 MHz, DMSO-d₆) δ 0.53-0.55 (2H, m), 0.69-0.74 (2H, m), 1.19 (3H, d), 1.60-1.63 (2H, m), 1.88-1.92 (2H, m), 3.12-3.21 (1H, m), 3.44 (1H, d), 3.60 (1H, d), 3.74 (1H, d), 3.95 (1H, d), 4.14-4.24 (1H, m), 4.40-4.54 (1H, m), 6.77 (1H, s), 7.44 (2H, t), 7.85 (2H, dd), 8.88 (2, s), 9.11 (1H, d), 10.11 (1H, s).

mTOR Kinase Assay (Echo): 0.11 μM

### EXAMPLE 14f

¹H NMR (400.13 MHz, DMSO-d₆) δ 1.19 (3H, d), 1.59-1.65 (2H, m), 1.89-1.92 (2H, m), 2.79(3H,d), 3.17 (1H, t), 3.46-3.63 (2H, m), 3.74 (1H, d), 3.95 (1H, d), 4.18 (1H, s), 4.46 (1H, s), 6.77 (1H, s), 7.43 (2H, t), 7.85 (2H, dd), 8.94 (2H, s), 10.08 (1H, s).

mTOR Kinase Assay (Echo): 0.189 μM

### EXAMPLE 14g

¹H NMR (400.13 MHz, DMSO-d₆) δ 1.19 (3H, d), 1.59-1.66 (2H, m), 1.88-1.92 (2H, m), 3.18 (1H, d), 3.30 (2H, q), 3.44 (1H, d), 3.49-3.54 (2H, m), 3.60 (1H, d), 3.72-3.98 (4H, m), 4.18(1H,s), 4.45(1H,s), 6.77 (1H, s), 7.43 (2H, t), 7.79-7.89 (2H, m), 8.91(2H,s), 9.13-9.20 (1H, m), 10.08 (1H, s).

mTOR Kinase Assay (Echo): 0.0857 μM

### EXAMPLE 14h

¹H NMR (400.13 MHz, DMSO-d₆) δ 1.20 (3H, d), 1.61-1.65 (2H, m), 1.89-1.94 (2H, m), 3.19 (1H, d), 3.41-3.50 (1H, m), 3.61 (1H, d), 3.75 (1H, d), 3.82 (3H, s), 3.96 (1H, d), 4.22 (1H, s), 4.48 (1H, s), 6.78 (1H, s), 7.46 (2H, t), 7.54 (1H, s), 7.86 (2H, dd), 7.90 (1H, s), 8.97 (2H, s), 10.47 (1H, s).

mTOR Kinase Assay (Echo): 0.14 μM

### EXAMPLE 14i

¹H NMR (400.13 MHz, DMSO-d₆) δ 0.46-0.51 (2H, m), 0.65-0.70 (2H, m), 1.18 (3H, d), 1.57-1.64 (2H, m), 1.86-1.92 (2H, m), 2.60-2.65 (1H, m), 3.14-3.19 (1H, m), 3.45 (1H, dd), 3.60 (1H, dd), 3.74 (1H, d), 3.92-3.99 (1H, m), 4.45 (1H, s), 6.73 (1H, s), 7.38-7.48 (2H, m), 7.84 (2H, dd), 8.05-8.16 (3H, m), 8.62 (1H, s), 9.37 (1H, s).

mTOR Kinase Assay (Echo): 0.0339 μM

### EXAMPLE 14j

¹H NMR (400.13 MHz, DMSO-d₆) δ 1.18 (3H, d), 1.57-1.64 (2H, m), 1.88-1.91 (2H, m), 2.74 (3H, d), 3.11-3.21 (1H, m), 3.42-3.50 (1H, m), 3.60 (1H, d), 3.74 (1H, d), 3.93-3.98 (1H, m), 4.13-4.19 (1H, m), 4.40-4.47 (1H, m), 6.71 (1H, s), 7.35 (1H, d), 7.43 (2H, t), 7.84 (2H, dd), 7.98-8.07 (2H, m), 8.67 (1H, s), 9.50 (1H, s).

mTOR Kinase Assay (Echo): 0.0129 μM

### EXAMPLE 14k

¹H NMR (400.13 MHz, DMSO-d₆) δ 1.19 (3H, d), 1.58-1.62 (2H, m), 1.88-1.91 (2H, m), 2.19 (6H, s), 2.32-2.35 (2H, m), 3.17 (1H, d), 3.24-3.29 (2H, m), 3.44 (1H, d), 3.60 (1H, d), 3.74 (1H, d), 3.95 (1H, d), 4.13-4.21 (1H, m), 4.41-4.49 (1H, m), 6.71 (1H, s), 7.39-7.46 (3H, m), 7.84 (2H, dd), 7.98 (1H, s), 8.06 (1H, d), 8.65 (1H, s), 9.48 (1H, s).

mTOR Kinase Assay (Echo): 0.275 μM

### EXAMPLE 14l

¹H NMR (400.13 MHz, DMSO-d₆) δ 1.18 (3H, d), 1.59-1.65 (2H, m), 1.85-1.92 (2H, m), 3.12-3.21 (1H, m), 3.22-3.27 (2H, m), 3.43-3.50 (3H, m), 3.60 (1H, d), 3.74 (1H, d), 3.95 (1H, d), 4.17 (1H, s), 4.44 (1H, s), 4.79 (1H, t), 6.71 (1H, s), 7.37-7.46 (3H, m), 7.80-7.89 (2H, m), 7.84 (3H, dd), 8.04 (1H, d), 8.16 (1H, s), 8.70 (1H, s), 9.49 (1H, s).

mTOR Kinase Assay (Echo): 0.0137 μM

### EXAMPLE 14m

¹H NMR (400.13 MHz, DMSO-d₆) δ 1.19 (3H, d), 1.58-1.65 (2H, m), 1.88-1.92 (2H, m), 3.13-3.22 (1H, m), 3.42-3.49 (1H, m), 3.61 (1H, d), 3.75 (1H, d), 3.81 (3H, s), 3.96 (1H, d), 4.14-4.24 (1H, m), 4.43-4.51 (1H, m), 6.73 (1H, s), 7.42-7.50 (3H, m), 7.82-7.90 (2H, m), 8.07-8.20 (1H, m), 8.71 (1H, s), 9.72 (1H, s), 10.28 (1H, s).

mTOR Kinase Assay (Echo): 0.0143 μM

### EXAMPLE 14n

¹H NMR (400.13 MHz, DMSO-d₆) δ 0.44-0.51 (2H, m), 0.66-0.71 (2H, m), 1.22 (3H, d), 1.50-1.61 (2H, m), 1.78-1.85 (2H, m), 2.40-2.47 (2H, m), 2.60-2.66 (1H, m), 2.69-2.78 (2H, m), 2.90 (3H,s), 3.15-3.27 (1H, m), 3.44-3.54 (1H, m), 3.64 (1H, d), 3.76 (1H, d), 3.98 (1H, d), 4.29 (1H, s), 4.58 (1H, s), 6.84 (1H, s), 7.51 (1H, d), 8.20 (1H, s), 8.53 (1H, d), 9.12 (1H, s), 9.37 (1H, s).

mTOR Kinase Assay (Echo): 0.0363 μM

### EXAMPLE 14o

¹H NMR (400.13 MHz, DMSO-d₆) δ 1.23 (3H, d), 1.51-1.60 (2H, m), 1.75-1.86 (2H, m), 2.40-2.47 (2H, m), 2.71-2.79 (5H, m), 2.90(3H,s), 3.22 (1H, d), 3.48 (1H, d), 3.64 (1H, d), 3.76 (1H, d), 3.97 (1H, d), 4.21-4.32 (1H, m), 4.58 (1H, s), 6.84 (1H, s), 7.42 (1H, d), 8.18 (1H, s), 8.52 (1H, d), 9.14 (1H, s), 9.52 (1H, s).

mTOR Kinase Assay (Echo): 0.0217 μM

### EXAMPLE 14p

¹H NMR (400.13 MHz, DMSO-d₆) δ 1.23 (3H, d), 1.54-1.61 (2H, m), 1.78-1.84 (2H, m), 2.18 (6H, s), 2.32-2.38 (2H, m), 2.38-2.47 (2H, m), 2.69-2.79 (2H, m), 2.90 (3H,s), 3.14-3.29 (3H, m), 3.43-3.55 (1H, m), 3.64 (1H, dd), 3.76 (1H, d), 3.97 (1H, dd), 4.26 (1H, s), 4.58 (1H, s), 6.84 (1H, s), 7.54 (1H, d), 8.03 (1H, s), 8.52 (1H, d), 9.12 (1H, s), 9.48 (1H, s).

mTOR Kinase Assay (Echo): 0.537 μM

### EXAMPLE 14q

¹H NMR (400.13 MHz, DMSO-d₆) δ 1.23 (3H, d), 1.51-1.61 (2H, m), 1.76-1.86 (2H, m), 2.37-2.46 (2H, m), 2.69-2.78 (2H, m), 2.90 (3H, s), 3.17-3.29 (3H, m), 3.42-3.54 (3H, m), 3.64 (1H, dd), 3.76 (1H, d), 3.92-4.01 (1H, m), 4.26 (1H, s), 4.57 (1H, s), 4.80 (1H, t), 6.84 (1H, s), 7.47 (1H, d), 8.31 (1H, s), 8.53 (1H, d), 9.12 (1H, s), 9.50 (1H, s).

mTOR Kinase Assay (Echo): 0.0265 μM

### EXAMPLE 14r

¹H NMR (400.13 MHz, DMSO-d₆) δ 1.24 (3H, d), 1.55-1.60 (2H, m), 1.79-1.85 (2H, m), 2.39-2.47 (2H, m), 2.71-2.81 (2H, m), 2.90 (3H, s), 3.17-3.26 (1H, m), 3.46-3.55 (1H, m), 3.65 (2H, d), 3.78 (4H, d), 3.98 (1H, d), 4.28 (1H, s), 4.59 (1H, s), 6.86 (1H, s), 7.50 (2H, d), 7.87 (1H, s), 8.58 (1H, d), 9.20 (1H, s), 9.73 (1H, s), 10.34 (1H, s).

mTOR Kinase Assay (Echo): 0.0103 μM

### EXAMPLE 14s

¹H NMR (400.13 MHz, DMSO-d₆) δ 0.51-0.58 (2H, m), 0.67-0.76 (2H, m), 1.23 (3H, d), 1.50-1.62 (2H, m), 1.76-1.88 (2H, m), 2.31-2.49 (2H, m), 2.64-2.79 (2H, m), 2.90 (3H, s), 3.15-3.26 (1H, m), 3.49 (1H, t), 3.64 (1H, d), 3.76 (1H, d), 3.97 (1H, d), 4.29 (1H, s), 4.59 (1H, s), 6.88 (1H, s), 9.15 (1H, s), 9.36 (2H, s), 10.13 (1H, s).

mTOR Kinase Assay (Echo): 0. 122 μM

### EXAMPLE 14t

¹H NMR (400.13 MHz, DMSO-d₆) δ 1.21 (3H, d), 1.56 (2H,m), 1.77-1.87 (2H, m), 2.34 (3H, d), 2.38-2.46 (2H, m), 2.68-2.76 (2H, m), 2.80 (3H, d), 3.16-3.23 (1H, m), 3.46-3.51 (1H, m), 3.64 (1H, d), 3.76 (1H, d), 3.96 (1H, d), 4.22-4.35 (1H, m), 4.54-4.68 (1H, m), 6.89 (1H, s), 8.97-9.07 (1H, m), 9.36 (2H, s), 10.11 (1H, s).

mTOR Kinase Assay (Echo): 0.197 μM

### EXAMPLE 14u

¹H NMR (400.13 MHz, DMSO-d₆) δ 1.23 (3H, d), 1.52-1.59 (2H, m), 1.78-1.85 (2H, m), 2.38-2.52 (4H, m), 2.69-2.79 (2H, m), 3.20 (1H, d), 3.32 (3H, s), 3.42-3.47 (1H, m), 3.49-3.55 (2H, m), 3.61-3.67 (1H, m), 3.76 (1H, d), 3.97 (1H, d), 4.30 (1H, s), 4.61 (1H, s), 4.83 (1H, t), 6.89 (1H, s), 9.15-9.26 (1H, m), 9.36 (2H, s), 10.11 (1H, s).

mTOR Kinase Assay (Echo): 0.123 μM

### EXAMPLE 14v

¹H NMR (400.13 MHz, DMSO-d₆) δ 1.24 (3H, d), 1.54-1.60 (2H, m), 1.79-1.86 (2H, m), 2.38-2.47 (2H, m), 2.65-2.81 (5H, m), 3.11-3.27 (1H, m), 3.46-3.53 (1H, m), 3.65 (1H, d), 3.76 (1H, d), 3.81 (3H, s), 3.98 (1H, d), 4.27-4.35 (1H, m), 4.57-4.65 (1H, m), 6.91 (1H, s), 7.55 (1H, s), 7.92 (1H, s), 9.08 (1H, s), 9.44 (2H, s), 10.49 (1H, s).

mTOR Kinase Assay (Echo): 0.0823 μM

### EXAMPLE 14w

¹H NMR (400.13 MHz, DMSO-d₆) δ 0.52-0.57 (2H, m), 0.68-0.74 (2H, m), 0.91-0.95 (2H, m), 0.99-1.04 (2H, m), 1.23 (3H, d), 1.56-1.61 (2H, m), 1.63-1.68 (2H, m), 2.65-2.73 (1H, m), 2.95-3.03 (1H, m), 3.22 (1H, dd), 3.43-3.53 (1H, m), 3.63 (1H, dd), 3.76 (1H, d), 3.97 (1H, dd), 4.23 (1H, s), 4.56 (1H, s), 6.95 (1H, s), 9.15 (1H, s), 9.33 (2H, s), 10.13 (1H, s).

mTOR Kinase Assay (Echo): 0.208 μM

### EXAMPLE 14x

¹H NMR (400.13 MHz, DMSO-d₆) δ 0.89-0.96 (2H, m), 1.00-1.06 (2H, m), 1.24 (3H, d), 1.56-1.61 (2H, m), 1.63-1.67 (2H, m), 2.80 (3H, d), 2.98-3.05 (1H, m), 3.19-3.25 (1H, m), 3.48 (1H, dd), 3.57-3.66 (1H, m), 3.70-3.80 (1H, m), 3.90-4.00 (1H, m), 4.21 (1H, s), 4.56 (1H, s), 6.94 (1H, s), 8.98 (1H, d), 9.33 (2H, s), 10.10 (1H, s).

mTOR Kinase Assay (Echo): 0.63 μM

### EXAMPLE 14y

¹H NMR (400.13 MHz, DMSO-d₆) δ 0.89-0.95 (2H, m), 1.00-1.05 (2H, m), 1.24 (3H, d), 1.57-1.61 (2H, m), 1.64-1.69 (2H, m), 2.98-3.04 (1H, m), 3.18-3.27 (1H, m), 3.29-3.34 (2H, m), 3.44-3.55 (3H, m), 3.63 (1H, dd), 3.76 (1H, d), 3.97 (1H, dd), 4.23 (1H, s), 4.56 (1H, s), 4.83 (1H, t), 6.95 (1H, s), 9.20 (1H, t), 9.35 (2H, s), 10.11 (1H, s).

mTOR Kinase Assay (Echo): 0.152 μM

### EXAMPLE 14z

¹H NMR (400.13 MHz, DMSO-d₆) δ 0.92-0.95 (2H, m), 0.98-1.07 (2H, m), 1.23 (3H, d), 1.58-1.61 (2H, m), 1.62-1.68 (2H, m), 2.97-3.03 (1H, m), 3.23 (1H, td), 3.49 (1H, td), 3.64 (1H, d), 3.76-3.85 (4H, m), 3.98 (1H, dd), 4.24 (1H, s), 4.57 (1H, s), 6.96 (1H, s), 7.56 (1H, s), 7.91 (1H, s), 9.41 (2H, s), 10.49 (1H, s), 11.15 (1H, s).

mTOR Kinase Assay (Echo): 0.171 [M

### EXAMPLE 14aa

¹H NMR (400.13 MHz, DMSO-d₆) δ 1.18 (3H, d), 1.53-1.61 (2H, m), 1.74-1.79 (2H, m), 3.10-3.19 (2H, m), 3.28-3.36 (4H, m), 3.45 (1H, t), 3.48-3.56 (2H, m), 3.60 (1H, d), 3.74 (1H, d), 3.95 (1H, d), 4.10 (1H, s), 4.34 (1H, s), 4.75 (1H, t), 4.85 (1H, t), 6.62 (2H, d), 6.65-6.70 (2H, m), 7.41 (2H, d), 9.11 (2H, s), 9.20 (1H, t), 10.04 (1H, s).

mTOR Kinase Assay (Echo): 0.0186 μM

The preparation of phenyl N-[5-[4-(1-cyclopropylsulfonylcyclopropyl)-6-[(3S)-3-methylmorpholin-4-yl]pyrimidin-2-yl]pyridin-2-yl]carbamate is described below.

### Phenyl N-[5-[4-(1-cyclopropylsulfonylcyclopropyl)-6-[(3S)-3-methylmorpholin-4-yl]pyrimidin-2-yl]pyridin-2-yl]carbamate

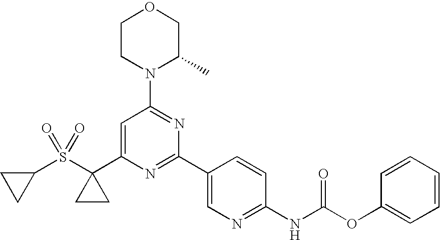

Phenyl chloroformate (0.543 mL, 4.33 mmol) was added to a mixture of 5-[4-(1-cyclopropylsulfonylcyclopropyl)-6-[(3S)-3-methylmorpholin-4-yl]pyrimidin-2-yl]pyridin-2-amine (1.2 g, 2.89 mmol) and sodium hydrogen carbonate (0.364 g, 4.33 mmol) in dioxane (30 mL) at 5° C. under nitrogen. The resulting mixture was stirred at RT for 1 hour, then at 40° C. for 3 hours and again at RT overnight. Additional phenyl chloroformate (1 mL) was 1o added and the mixture heated at 40° C. until complete. The reaction mixture was diluted with ethyl acetate (200 mL) and washed with water (2×100 mL). The organic layer was dried (Na₂SO₄), filtered and evaporated to afford crude product which was then purified chromatography on silica, eluting with 10 to 80% ethyl acetate in isohexane, to give a mixture of the desired material and material where an additional carbamate group was present (phenyl N-[5-[4-(1-cyclopropylsulfonylcyclopropyl)-6-[(3S)-3-methylmorpholin-4-yl]pyrimidin-2-yl]pyridin-2-yl]-N-phenoxycarbonylcarbamate). This material (1 g) was used in the subsequent step without further attempts to purify.

LCMS Spectrum: m/z (ESI+)(M+H)+=536; HPLC tR=2.65 min.

### 5-[4-(1-Cyclopropylsulfonylcyclopropyl)-6-[(3S)-3-methylmorpholin-4-yl]pyrimidin-2-yl]pyridin-2-amine

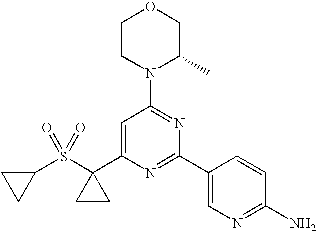

Bis(triphenylphosphine)palladium(II) chloride (118 mg, 0.17 mmol) was added to 2-chloro-4-(1-cyclopropylsulfonylcyclopropyl)-6-[(3S)-3-methylmorpholin-4-yl]pyrimidine (900 mg, 2.51 mmol), 2-amino-5-(4,4,5,5-tetramethyl-1,3,2-dioxaborolan-2-yl)pyridine (830 mg, 3.77 mmol) and sodium carbonate (5.03 mL, 10.06 mmol) in a mixture of DMF (6 mL), DME (8 mL), water (2 mL) and ethanol (1.5 mL) at RT. The resulting mixture was stirred at 90° C. for 18 hours then the reaction mixture diluted with ethyl acetate (200 mL) and washed with water (2×100 mL). The organic layer was dried (Na₂SO₄), filtered and evaporated to afford crude product. The crude product was purified by chromatography on silica, eluting with 0 to 5% methanol in ethyl acetate. The material was further purified by ion exchange chromatography using an SCX column with the desired material eluted using 7M ammonia in methanol, to give the pure desired material as a white solid (1.2 g).

NMR Spectrum: ¹H NMR (400.13 MHz, DMSO-d₆) δ 0.92-0.95 (2H, m), 1.00-1.04 (2H, m), 1.22 (3H, d), 1.53-1.59 (2H, m), 1.62-1.65 (2H, m), 2.94-3.02 (1H, m), 3.19 (1H, td), 3.48 (1H, td), 3.63 (1H, d), 3.75 (1H, d), 3.96 (1H, d), 4.17 (1H, d), 4.53 (1H, s), 6.39 (2H, s), 6.50 (1H, d), 6.81 (1H, s), 8.23 (1H, d), 8.89 (1H, d)

LCMS Spectrum: m/z (ESI+)(M+H)+=416; HPLC tR=1.81 min.

The preparation of 2-chloro-4-(1-cyclopropylsulfonylcyclopropyl)-6-[(3S)-3-methylmorpholin-4-yl]pyrimidine was described earlier.

The preparation of phenyl N-[5-[4-[1-(4-fluorophenyl)sulfonylcyclopropyl]-6-[(3S)-3-methylmorpholin-4-yl]pyrimidin-2-yl]pyrimidin-2-yl]carbamate is described below.

### Phenyl N-[5-[4-[1-(4-fluorophenyl)sulfonylcyclopropyl]-6-[(3S)-3-methylmorpholin-4-yl]pyrimidin-2-yl]pyrimidin-2-yl]carbamate

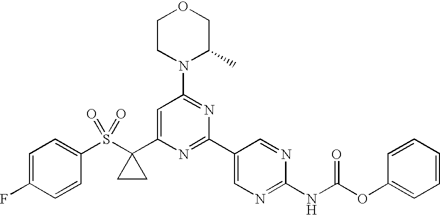

Phenyl chloroformate (0.306 mL, 2.44 mmol) was added to a mixture of 5-[4-[1-(4-fluorophenyl)sulfonylcyclopropyl]-6-[(3S)-3-methylmorpholin-4-yl]pyrimidin-2-yl]pyrimidin-2-amine (765 mg, 1.63 mmol) and sodium hydrogen carbonate (205 mg, 2.44 mmol) in dioxane (10 mL) at 5° C. under nitrogen. The resulting mixture was stirred at RT overnight. Additional phenyl chloroformate (0.2 mL) was added and reaction left to stir overnight. The reaction mixture was diluted with ethyl acetate (100 mL), washed with water (2×100 mL), and a saturated brine solution (50 mL). The organic layer was dried (Na₂SO₄), filtered and evaporated to afford a gum. The crude gum was triturated with diethyl ether to give a solid which was collected by filtration and dried under vacuum to give the desired material as a yellow solid (1.0 g). No further purification was performed at this stage.

LCMS Spectrum: m/z (ESI+)(M+H)+=591; HPLC tR=2.61 min.

### 5-[4-[1-(4-Fluorophenyl)sulfonylcyclopropyl]-6-[(3S)-3-methylmorpholin-4-yl]pyrimidin-2-yl]pyrimidin-2-amine

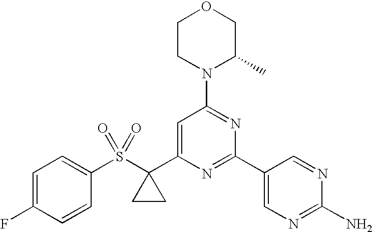

Bis(triphenylphosphine)palladium(II) chloride (0.143 g, 0.20 mmol) was added to 2-chloro-4-[1-(4-fluorophenyl)sulfonylcyclopropyl]-6-[(3S)-3-methylmorpholin-4-yl]pyrimidine (1.25 g 3.03 mmol), 2-amino-5-(4,4,5,5-tetramethyl-1,3,2-dioxaborolan-2-yl)pyridine (0.868 g, 3.95 mmol) and sodium carbonate (3 mL, 6.00 mmol) in a mixture of DMF (3 mL), DME (8 mL), water (2 mL) and ethanol (1.5 mL) at RT and the atmosphere replaced with nitrogen. The mixture was stirred at 90° C. for 5 hours the left to stir at RT overnight. The reaction mixture was diluted with ethyl acetate (200 mL), washed with water (2×100 mL), and the organic layer dried (Na₂SO₄), filtered and evaporated to afford crude product. The crude solid was triturated with DCM to give a solid which was collected by filtration and dried under vacuum to give the desired material as a beige solid (1.1 g).

NMR Spectrum: ¹H NMR (400.13 MHz, DMSO-d₆) δ 1.18 (3H, d), 1.56-1.61 (2H, m), 1.86-1.90 (2H, m), 3.15 (1H, td), 3.45 (1H, td), 3.60 (1H, dd), 3.74 (1H, d), 3.95 (1H, dd), 4.14 (1H, d), 4.46 (1H, s), 6.69 (1H, s), 7.13 (2H, s), 7.41 (2H, t), 7.80-7.85 (2H, m), 8.65 (2H,s )

LCMS Spectrum: m/z (ESI+)(M+H)+=471; HPLC tR=1.94 min.

The preparation of 2-chloro-4-[1-(4-fluorophenyl)sulfonylcyclopropyl]-6-[(3S)-3-methylmorpholin-4-yl]pyrimidine was described earlier.

The preparation of phenyl N-[5-[4-[1-(4-fluorophenyl)sulfonylcyclopropyl]-6-[(3S)-3-methylmorpholin-4-yl]pyrimidin-2-yl]pyridin-2-yl]carbamate is described below.

### Phenyl N-[5-[4-[1-(4-fluorophenyl)sulfonylcyclopropyl]-6-[(3S)-3-methylmorpholin-4-yl]pyrimidin-2-yl]pyridin-2-yl]carbamate

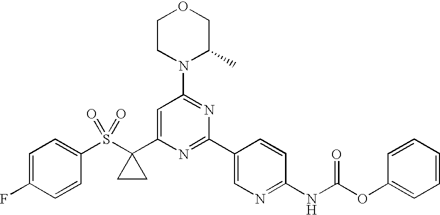

Phenyl chloroformate (0.962 mL, 7.67 mmol) was added to a mixture of 5-[4-[1-(4-fluorophenyl)sulfonylcyclopropyl]-6-[(3S)-3-methylmorpholin-4-yl]pyrimidin-2-yl]pyridin-2-amine (900 mg, 1.92 mmol) and sodium hydrogen carbonate (242 mg, 2.88 mmol) in dioxane (50 mL) at 5° C. under nitrogen. The resulting mixture was stirred at RT for 3 days, additional phenyl chloroformate (2 mL) added and the reaction stirred at RT for an additional 16 hours. The reaction mixture was diluted with ethyl acetate (200 mL), washed with water (100 mL), and the organic layer was dried (Na₂SO₄), filtered and evaporated to afford crude product. The crude solid was triturated with diethyl ether to give a solid which was collected by filtration and dried under vacuum to give the desired material as a cream solid (900 mg).

LCMS Spectrum: m/z (ESI+)(M+H)+=591; HPLC tR=2.63 min.

### 5-[4-[1-(4-Fluorophenyl)sulfonylcyclopropyl]-6-[(3S)-3-methylmorpholin-4-yl]pyrimidin-2-yl]pyridin-2-amine

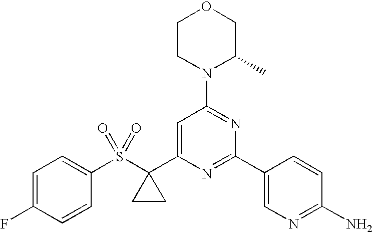

Bis(triphenylphosphine)palladium(II) chloride (0.143 g, 0.20 mmol) was added to (2-chloro-4-[1-(4-fluorophenyl)sulfonylcyclopropyl]-6-[(3S)-3-methylmorpholin-4-yl]pyrimidine (1.25 g, 3.03 mmol), 2-amino-5-(4,4,5,5-tetramethyl-1,3,2-dioxaborolan-2-yl)pyridine (0.868 g, 3.95 mmol) and sodium carbonate (3 mL, 6.00 mmol) in a mixture of DMF (3 mL), DME (8 mL), water (2 mL) and ethanol (1.5 mL) at RT under at nitrogen atmosphere. The resulting mixture was stirred at 90° C. for 5 hours then at RT for 16 hours. The reaction mixture was diluted with ethyl acetate (200 mL), washed with water (2×100 mL), and the organic layer was dried (Na₂SO₄), filtered and evaporated to afford crude product. The crude product was purified by flash silica chromatography, eluting with 20 to 80% ethyl acetate in isohexane. The isolated material was further purified by ion exchange chromatography using an SCX column with the desired material eluted from the column using 7M ammonia in methanol. The desired material was isolated as as a white solid (0.94 g).

NMR Spectrum: ¹H NMR (400.13 MHz, DMSO-d₆) δ 1.17 (3H, d), 1.57-1.61 (2H, m), 1.86-1.89 (2H, m), 3.09-3.17 (1H, m), 3.41-3.49 (1H, m), 3.60 (1H, dd), 3.74 (1H, d), 3.96 (1H, d), 4.11 (1H, d), 4.43 (1H, s), 6.35-6.41 (3H, m), 6.63 (1H, s), 7.38-7.45 (2H, m), 7.78-7.85 (3H, m), 8.51 (1H, d)

LCMS Spectrum: m/z (ESI+)(M+H)+=470; HPLC tR=2.06 min.

The preparation of 2-chloro-4-[1-(4-fluorophenyl)sulfonylcyclopropyl]-6-[(3S)-3-methylmorpholin-4-yl]pyrimidine was described earlier.

The preparation of phenyl N-[5-[4-[(3S)-3-methylmorpholin-4-yl]-6-(1-methylsulfonylcyclopentyl)pyrimidin-2-yl]pyridin-2-yl]carbamate is described below.

### Phenyl N-[5-[4-[(3S)-3-methylmorpholin-4-yl]-6-(1-methylsulfonylcyclopentyl)pyrimidin-2-yl]pyridin-2-yl]carbamate

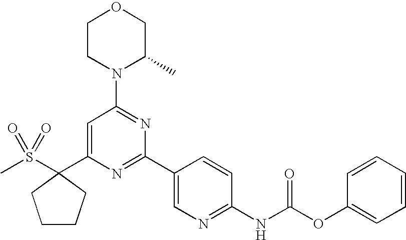

Phenyl chloroformate (1.23 mL, 9.82 mmol) was added to 5-[4-[(3S)-3-methylmorpholin-4-yl]-6-(1-methylsulfonylcyclopentyl)pyrimidin-2-yl]pyridin-2-amine (1.025 g, 2.45 mmol) and sodium hydrogen carbonate (0.309 g, 3.68 mmol) in dioxane (50 mL) at 5° C. under nitrogen. The resulting mixture was stirred at RT for 3 days then additional phenyl chloroformate (2 mL) added and the reaction left to stir at 35° C. for 3 hours. The reaction mixture was diluted with ethyl acetate (100 mL), washed with water (2×100 mL), and the organic layer dried (Na₂SO₄), filtered and evaporated to afford crude product. The crude liquid was triturated with diethyl ether to give a solid which was collected by filtration and dried under vacuum to give the desired material as a cream solid (1.2 g).

LCMS Spectrum: m/z (ESI+)(M+H)+=538; HPLC tR=2.89 min.

### 5-[4-[(3S)-3-Methylmorpholin-4-yl]-6-(1-methylsulfonylcyclopentyl)pyrimidin-2-yl]pyridin-2-amine

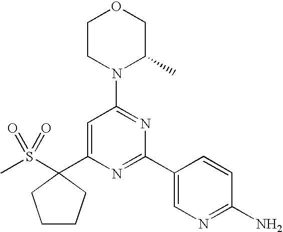

Bis(triphenylphosphine)palladium(II) chloride (0.261 g, 0.37 mmol) was added to 2-chloro-4-[(3S)-3-methylmorpholin-4-yl]-6-(1-methylsulfonylcyclopentyl)pyrimidine (2 g, 5.56 mmol), 2-amino-5-(4,4,5,5-tetramethyl-1,3,2-dioxaborolan-2-yl)pyridine (1.835 g, 8.34 mmol) and aqueous sodium carbonate solution (1 1.12 mL, 22.23 mmol) in a mixture of DMF (6 mL), DME (8 mL), water (2 mL) and ethanol (1.5 mL) at RT under an atmosphere of nitrogen. The resulting mixture was stirred at 90° C. for 18 hours. The reaction mixture was diluted with ethyl acetate (200 mL), washed with water (2×100 mL) and the organic layer dried (Na₂SO₄), filtered and evaporated to afford crude product. The crude product was purified by flash silica chromatography, eluting with 10 to 100% ethyl acetate in isohexane. The isolated material was dissolved in methanol, the solid removed and the filtrate purified by ion exchange chromatography using an SCX column with the desired product eluted from the column using 7M ammonia in methanol. The desired material was isolated as a white solid (2.2 g).

NMR Spectrum: ¹H NMR (400.13 MHz, DMSO-d₆) δ 1.22 (3H, d), 1.54-1.60 (2H, m), 1.78-1.85 (2H, m), 2.37-2.46 (2H, m), 2.65-2.78 (2H, m), 2.90 (3H, s), 3.18 (1H, dd), 3.49 (1H, td), 3.64 (1H, dd), 3.75 (1H, d), 3.97 (1H, dd), 4.22 (1H, d), 4.56 (1H, s), 6.39 (2H, s), 6.50 (1H, d), 6.75 (1H, s), 8.25 (1H, d), 8.92 (1H, s)

LCMS Spectrum: m/z (ESI+)(M+H)+=418; HPLC tR=2.05 min.

The preparation of 2-chloro-4-[(3S)-3-methylmorpholin-4-yl]-6-(1-methylsulfonylcyclopentyl)pyrimidine was described earlier.

The preparation of phenyl N-[5-[4-[(3S)-3-methylmorpholin-4-yl]-6-(1-methylsulfonylcyclopentyl)pyrimidin-2-yl]pyrimidin-2-yl]carbamate is described below.

### Phenyl N-[5-[4-[(3S)-3-methylmorpholin-4-yl]-6-(1-methylsulfonylcyclopentyl)pyrimidin-2-yl]pyrimidin-2-yl]carbamate

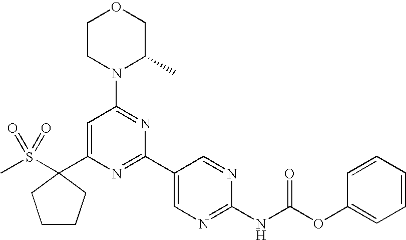

Phenyl chloroformate (0.315 mL, 2.51 mmol) was added to 5-[4-[(3S)-3-methylmorpholin-4-yl]-6-(1-methylsulfonylcyclopentyl)pyrimidin-2-yl]pyrimidin-2-amine (700 mg, 1.67 mmol), sodium hydrogen carbonate (211 mg, 2.51 mmol) in dioxane (20 mL) at 5° C. under nitrogen. The resulting mixture was stirred at RT for 48 hours. The reaction mixture was diluted with ethyl acetate (100 mL), and washed with water (2×100 mL). The organic layer was dried (Na₂SO₄), filtered and evaporated to afford crude product which was triturated with diethyl ether to give the desired material as a yellow solid (790 mg).

LCMS Spectrum: m/z (ESI+)(M+H)+=539; HPLC tR=2.52 min.

### 5-[4-[(3S)-3-Methylmorpholin-4-yl]-6-(1-methylsulfonylcyclopentyl)pyrimidin-2-yl]pyrimidin-2-amine

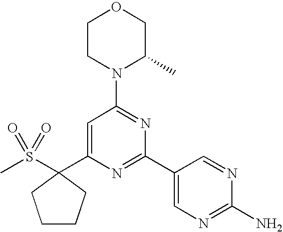

Bis(triphenylphosphine)palladium(II) chloride (0.261 g, 0.37 mmol) was added to 2-chloro-4-[(3S)-3-methylmorpholin-4-yl]-6-(1-methylsulfonylcyclopentyl)pyrimidine (2 g, 5.56 mmol), 5-(4,4,5,5-tetramethyl-1,3,2-dioxaborolan-2-yl)-pyrimidin-2-ylamine (1.843 g, 8.34 mmol) and aqueous sodium carbonate solution (1 1.12 mL, 22.23 mmol) in a mixture of DMF (6 mL), DME (8 mL), water (2 mL) and ethanol (1.5 mL) at RT. The resulting mixture was stirred at 90° C. for 18 hours under a nitrogen atmosphere. The reaction mixture was diluted with ethyl acetate (200 mL), and washed with water (2×100 mL). The organic layer was dried (Na₂SO₄), filtered and evaporated to afford crude product. The crude product was purified by flash silica chromatography, elution gradient 0 to 3% methanol in ethyl acetate. The isolated material was triturated with a mixture of diethyl ether and isohexane and filtered to give the desired material as a cream solid (2.0 g).

NMR Spectrum: ¹H NMR (400.13 MHz, DMSO-d₆) δ 1.22 (3H, d), 1.53-1.60 (2H, m), 1.76-1.83 (2H, m), 2.37-2.47 (2H, m), 2.64-2.76 (2H, m), 2.91 (3H, s), 3.15-3.23 (1H, m), 3.49 (1H, td), 3.63 (1H, dd), 3.75 (1H, d), 3.96 (1H, dd), 4.24 (1H, d), 4.59 (1H, s), 6.82 (1H, s), 7.14 (2H, s), 9.09 (1H, s)

LCMS Spectrum: m/z (ESI+)(M+H)+=419; HPLC tR=1.83 min.

The preparation of 2-chloro-4-[(3S)-3-methylmorpholin-4-yl]-6-(1-methylsulfonylcyclopentyl)pyrimidine was described earlier.

The preparation of phenyl N-[5-[4-(1-cyclopropylsulfonylcyclopropyl)-6-[(3S)-3-methylmorpholin-4-yl]pyrimidin-2-yl]pyrimidin-2-yl]carbamate is described below.

### Phenyl N-[5-[4-(1-cyclopropylsulfonylcyclopropyl)-6-[ (3S)-3-methylmorpholin-4-yl]pyrimidin-2-yl]pyrimidin-2-yl]carbamate

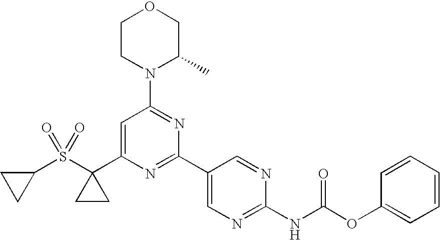

Phenyl chloroformate (0.976 mL, 7.78 mmol) was added dropwise to 5-[4-(1-cyclopropylsulfonylcyclopropyl)-6-[(3S)-3-methylmorpholin-4-yl]pyrimidin-2-yl]pyrimidin-2-amine (2.16 g, 5.19 mmol), sodium hydrogen carbonate (0.654 g, 7.78 mmol) in dioxane (30 mL) cooled to 10° C. under nitrogen. The resulting mixture was stirred at RT for 48 hours. The reaction mixture was diluted with ethyl acetate (150 ML), and washed with water (150 mL followed by 125 mL). The organic layer was dried (Na₂SO₄), filtered and evaporated to afford crude product which was purified by trituration with diethyl ether to give the desired material as a yellow solid (2.5 g).

LCMS Spectrum: m/z (ESI+)(M+H)+=537; HPLC tR=2.33 min.

### 5-[4-(1-Cyclopropylsulfonylcyclopropyl)-6-[(3S)-3-methylmorpholin-4-yl]pyrimidin-2-yl]pyrimidin-2-amine

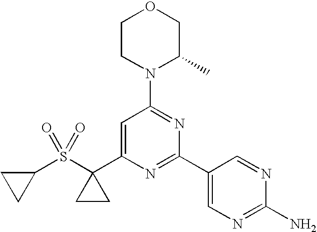

Bis(triphenylphosphine)palladium(II) chloride (0.381 g, 0.54 mmol) was added to 2-chloro-4-(1-cyclopropylsulfonylcyclopropyl)-6-[(3S)-3-methylmorpholin-4-yl]pyrimidine (2.9 g, 8.10 mmol), 5-(4,4,5,5-tetramethyl-1,3,2-dioxaborolan-2-yl)-pyrimidin-2-ylamine (2.69 g, 12.16 mmol) and aqueous sodium carbonate solution (4 mL, 8.00 mmol) in a mixture of DMF (18 mL), DME (8 mL), water (2 mL) and ethanol (1.5 mL) at RT. The resulting mixture was stirred at 90° C. for 18 hours under a nitrogen atmosphere. The reaction mixture was diluted with ethyl acetate (200 mL), and washed with water (2×100 mL). The organic layer was dried (Na₂SO₄), filtered and evaporated to afford crude product. The crude product was purified by flash silica chromatography, elution gradient 0 to 7% methanol in ethyl acetate. The isolated material was further purified by ion exchange chromatography using an SCX column, eluting with methanol followed by 7M ammonia in methanol, to give the desired material as a white solid (2.16 g).

NMR Spectrum: ¹H NMR (400.13 MHz, DMSO-d₆) δ 0.90-0.94 (2H, m), 0.99-1.04 (2H, m), 1.22 (3H, d), 1.54-1.58 (2H, m), 1.60-1.65 (2H, m), 2.95-3.02 (1H, m), 3.16-3.23 (1H, m), 3.47 (1H, td), 3.62 (1H, dd), 3.75 (1H, d), 3.96 (1H, dd), 4.18 (1H, s), 4.50 (1H, s), 6.85 (1H, s), 7.16 (2H, s), 9.05 (2H, s)

LCMS Spectrum: m/z (ESI+)(M+H)+=417; HPLC tR=1.70 min.

The preparation of 2-chloro-4-(1-cyclopropylsulfonylcyclopropyl)-6-[(3S)-3-methylmorpholin-4-yl]pyrimidine was described earlier.

### EXAMPLE 15

### 3-Cyclopropyl-1-[4-[4-[1-(2-hydroxyethylsulfonyl)cyclopropyl]-6-[(3S)-3-methylmorpholin-4-yl]pyrimidin-2-yl]phenyl]urea

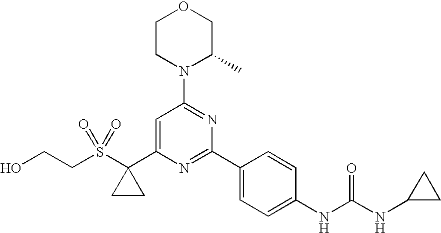

Bis (triphenylphosphine)palladium (II) chloride (6.84 mg, 0.00975 mmol) was added to 2-[1-[2-chloro-6-[(3S)-3-methylmorpholin-4-yl]pyrimidin-4-yl]cyclopropyl]sulfonylethoxy-tri(propan-2-yl)silane (0.101 g, 0. 19 mmol), 1-cyclopropyl-3-(4-(4,4,5,5-tetramethyl-1,3,2-dioxaborolan-2-yl)phenyl)urea (0.088 g, 0.29 mmol) and sodium carbonate (0.487 mL, 0.97 mmol) in a solvent mixture (18% DMF, 82% of a 7:3:2 mixture of DME:water:Ethanol) at RT under nitrogen. The resulting suspension was stirred at 80° C. for 17 hours. The solvent was removed and the residue partitioned between DCM and water. The organic layer was dried (MgSO₄), filtered and tetrabutylammonium fluoride (0.975 mL, 0.97 mmol) added to the filtrate and stirred for 2 hours. The mixture was washed with water, the organic layer concentrated in vacuo and purified by preparative HPLC, using decreasingly polar mixtures of water (containing 1% NH3) and MeCN as eluents, to give the desired material as a white solid (0.057 g).

NMR Spectrum: ¹H NMR (400 MHz, DMSO-d₆) δ 0.41-0.44 (2H, m), 0.63-0.68 (2H, m), 1.23-1.25 (3H, d), 1.53-1.56 (2H, m), 1.65-1.66 (2H, m), 2.55-2.59 (1H, m), 3.17-3.25 (1H, td), 3.56-3.52 (1H, td), 3.62-3.66 (3H, m), 3.75-3.78 (1H, d), 3.88-3.89 (2H, m), 3.96-4.00 (1H, dd), 4.20-4.23 (1H, d), 4.56 (1H, bs), 5.03 (1H, bs), 6.45-6.46 (1H, d), 6.78 (1H, s), 7.50-7.52 (2H, d), 8.20-8.22 (2H, d), 8.56 (1H, s).

LCMS Spectrum: m/z (ES+) (M+H)+=502; HPLC tR=1.86 min.

mTOR Kinase Assay (Echo): 0.00154 μM

The compound below was prepared in an analogous fashion using the appropriate boronate.

| | | | LCMS | Retention |
|---|---|---|---|---|
| Example | Structure | NAME | MH+ | time (min) |
| 15a | 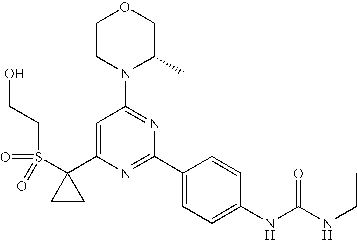 | 3-ethyl-1-[4-[4-[1-(2-hydroxyethylsulfonyl)cyclopropyl]-6-[(3S)-3-methylmorpholin-4-yl]pyrimidin-2-yl]phenyl]urea | 490 | 1.82 |

### EXAMPLE 15a

¹H NMR (400 MHz, DMSO-d₆) δ 1.05-1.09 (3H, t), 1.23-1.24 (3H, d), 1.53-1.56 (2H, m), 1.64-1.67 (2H, m), 3.10-3.17 (2H, m), 3.17-3.25 (1H, td), 3.45-3.52 (1H, td), 3.62-3.66 (3H, m), 3.75-3.78 (1H, d), 3.87-3.91 (2H, t), 3.96-3.99 (1H, dd), 4.19-4.22 (1H, d), 4.55 (1H, bs), 5.03 (1H, bs), 6.17-6.20 (1H, t), 6.77 (1H, s), 7.49-7.51 (2H, q), 8.19-8.21 (2H, q), 8.68 (1H, s).

mTOR Kinase Assay (Echo): 0.000591 μM

The preparation of 2-[1-[2-chloro-6-[(3S)-3-methylmorpholin-4-yl]pyrimidin-4-yl]cyclopropyl]sulfonylethoxy-tri(propan-2-yl)silane is described below.

### 2-[1-[2-Chloro-6-[(3S)-3-methylmorpholin-4-yl]pyrimidin-4-yl]cyclopropyl]sulfonylethoxy-tri(propan-2-yl)silane

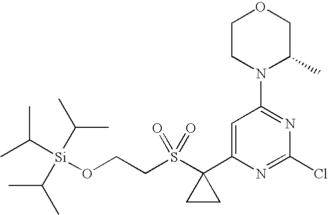

A 50% v/v aqueous sodium hydroxide solution (35 mL, 5.20 mmol) was added to 2-[[2-chloro-6-[(3S)-3-methylmorpholin-4-yl]pyrimidin-4-yl]methylsulfonyl]ethoxy-tri(propan-2-yl)silane (2.56 g, 5.20 mmol), 1,2-bibromoethane (1.345 mL, 15.61 mmol) and tetrabutylammonium bromide (0. 168 g, 0.52 mmol) in toluene (100 mL) at RT. The resulting slurry was stirred at 60° C. for 3 hours then the reaction mixture diluted with water and extracted sequentially with toluene and DCM. The organic layers were combined, dried (MgSO₄), filtered and evaporated to afford crude product. The crude product was purified by flash silica chromatography, eluting with 0 to 10% ethyl acetate in DCM, to give the desired material as a colourless oil which solidified on standing (0.304 g).

NMR Spectrum: ¹H NMR (400 MHz, CDCl₃) δ 1.01-1.11 (21H, m), 1.30-1.32 (3H, d), 1.47-1.50 (2H, q), 1.77-1.80 (2H, q), 3.24-3.31 (1H, td), 3.40-3.44 (2H, t), 3.48-3.55 (1H, td), 3.64-3.68 (1H, td), 3.75-3.78 (1H, d), 3.97-4.01 (2H, m), 4.10-4.14 (2H, t), 4.32 (1H, bs), 6.86 (1H, s).

LCMS Spectrum: m/z (ES+) (M+H)+=518; HPLC tR=3.86 min.

### 2-[[2-Chloro-6-[(3S)-3-methylmorpholin-4-yl]pyrimidin-4-yl]methylsulfonyl]ethoxy-tri(propan-2-yl)silane

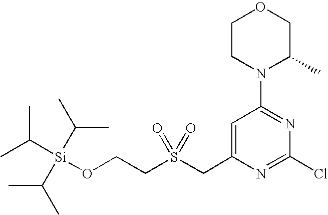

2-[[2-Chloro-6-[(3S)-3-methylmorpholin-4-yl]pyrimidin-4-yl]methylsulfonyl]ethanol (4.21 g, 12.53 mmol) was added to triisopropylsilyl chloride (3.22 mL, 15.03 mmol) and imidazole (2.046 g, 30.06 mmol) in DMF at RT. The resulting solution was stirred at RT overnight under a nitrogen atmosphere. The DMF was removed in vacuo and ethyl acetate added. The solids were removed by filtration and the filtrate concentrated in vacuo and purified by flash silica chromatography, eluting with 0 to 4% methanol in DCM. The isolated material was again purified by chromatography on silica, eluting with 0-10% ethyl acetate in DCM, to give the desired material to as a clear gum (4.15 g).

NMR Spectrum: ¹H NMR (400 MHz, CDCl₃) δ 1.03-1.15 (21H, m), 1.27-1.29 (3H, d), 3.20-3.28 (1H, td), 3.37-3.40 (2H, m), 3.45-3.52 (1H, td), 3.61-3.65 (1H, dd), 3.71-3.74 (1H, d), 3.93-4.04 (2H, m), 4.15-4.18 (2H, t), 4.28 (3H, s), 6.50 (1H, s).

LCMS Spectrum: m/z (ES+) (M+H)+=492; HPLC tR=3.72 min.

### 2-[[2-Chloro-6-[(3S)-3-methylmorpholin-4-yl]pyrimidin-4-yl]methylsulfonyl]ethanol

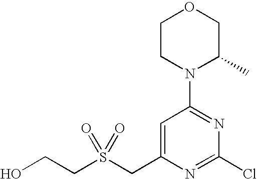

A solution of hydrogen peroxide (30% aqueous solution, 0.225 mL, 7.29 mmol) was added to a stirred solution of 2-chloro-4-[(3S)-3-methylmorpholin-4-yl]-6-[2-(oxan-2-yloxy)ethylsulfonylmethyl]pyrimidine (0.141 g, 0.36 mmol), sodium tungstate dihydrate (2.4 mg, 0.0073 mmol) in water (0.2 mL) and 2N sulfuric acid (0.011 mL) in 1,4-dioxane (1.4 mL) and methanol (1.4 mL) and the reaction stirred at 55° C. for 4 hours. Water (50 mL) was added and the reaction cooled. 10% Sodium metabisulfite aqueous solution was added and the mixture extracted with DCM. The organics were dried (MgSO₄), filtered and concentrated in vacuo to give the desired material as a opaque oil (0.198 g).

LCMS Spectrum: m/z (ES+) (M+H)+=336; HPLC tR=1.18 min.

### 2-Chloro-4-[(3S)-3-methylmorpholin-4-yl]-6-[2-(oxan-2-yloxy)ethylsulfonylmethyl]pyrimidine

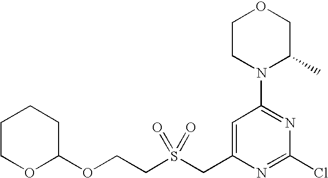

DIPEA (0.211 g, 1.63 mmol) was added dropwise to 2-chloro-4-(iodomethyl)-6-[(3S)-3-methylmorpholin-4-yl]pyrimidine (0.231 g, 0.65 mmol) and 2-(tetrahydro-2H-pyran-2-yloxy)ethanethiol (0.133 g, 0.82 mmol) in acetonitrile at RT. The resulting solution was stirred at RT for 1 hour. The solvent was removed and the residue partitioned between DCM and water. The organic layer was dried (MgSO₄), filtered and evaporated to afford crude product. The crude product was purified by flash silica chromatography, eluting with 0 to 2% methanol in DCM, to give the desired material as a colourless oil (0.141 g).

NMR Spectrum: ¹H NMR (400 MHz, CDCl3) δ 1.24-1.26 (3H, d), 1.40-1.55 (4H, m), 1.60-1.67 (1H, m), 1.69-1.77 (1H, m), 2.68-2.71 (2H, t), 3.17-3.24 (1H, td), 3.41-3.47 (2H, m), 3.50-3.58 (1H, m), 3.59 (2H, s), 3.62-3.63 (1H, d), 3.69-3.72 (1H, d), 3.76-3.86 (2H, m), 3.91-3.95 (1H, dd), 3.97 (1H, bs), 4.25 (1H, bs), 4.52-4.54 (1H, t), 6.44 (1H, s).

LCMS Spectrum: m/z (ES+) (M+H)+=386; HPLC tR=2.11 min.

The preparation of 2-chloro-4-(iodomethyl)-6-[(3S)-3-methylmorpholin-4-yl]pyrimidine was described earlier

### EXAMPLE 16

### 1-[4-[4-[1-(2-Hydroxyethylsulfonyl)cyclopropyl]-6-[(3S)-3-methylmorpholin-4-yl]pyrimidin-2-yl]phenyl]-3-methylurea

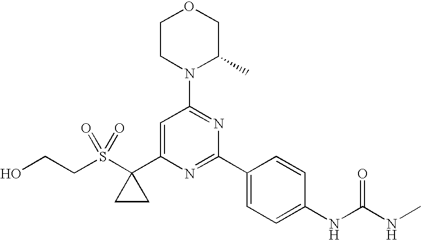

Methylamine (0.542 mL, 1.08 mmol) was added to (S)-phenyl 4-(4-(1-(2-hydroxyethylsulfonyl)cyclopropyl)-6-(3-methylmorpholino)pyrimidin-2-yl)phenylcarbamate (0.117 g, 0.22 mmol) and triethylamine (0.091 mL, 0.65 mmol) in DMF (3 mL) and the solution stirred at RT for 5 minutes. The crude product was purified by preparative HPLC, using decreasingly polar mixtures of water (containing 1% NH3) and MeCN as eluents, to give the desired material as a white solid (0.08 g).

NMR Spectrum: ¹H NMR (400 MHz, DMSO-d₆) δ 1.22-1.24 (3H, d), 1.53-1.56 (2H, m), 1.64-1.66 (2H, m), 2.66-2.67 (3H, d), 3.16-3.24 (1H, td), 3.45-3.51 (1H, td), 3.61-3.67 (3H, m), 3.75-3.78 (1H, d), 3.86-3.91 (2H, q), 3.96-3.99 (1H, dd), 4.20-4.23 (1H, d), 4.56 (1H, s), 5.06-5.09 (1H, t), 6.08-6.11 (1H, q), 6.77 (1H, s), 7.50-7.52 (2H, d), 8.18-8.21 (2H, d), 8.79 (1H, s).

LCMS Spectrum: m/z (ES+) (M+H)+=476; HPLC tR=1.69 min.

mTOR Kinase Assay (Echo): 0.00142 μM

The following compounds were prepared in an analogous fashion from phenyl N-[4-[4-[1-(2-hydroxyethylsulfonyl)cyclopropyl]-6-[(3S)-3-methylmorpholin-4-yl]pyrimidin-2-yl]phenyl]carbamate using the appropriate amine.

| Ex- | | | LCMS | Retention |
|---|---|---|---|---|
| ample | Structure | NAME | MH+ | time (min) |
| 16a | 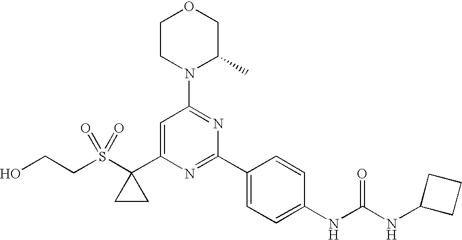 | 3-cyclobutyl-1-[4-[4-[1-(2-hydroxyethylsulfonyl)cyclopropyl]-6-[(3S)-3-methylmorpholin-4-yl]pyrimidin-2-yl]phenyl]urea | 516 | 2.11 |
| 16b | 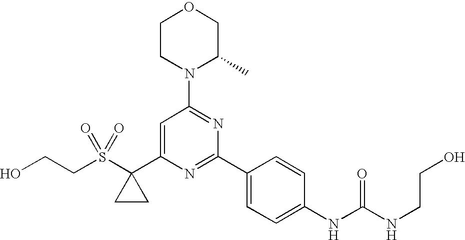 | 3-(2-hydroxyethyl)-1-[4-[4-[1-(2-hydroxyethylsulfonyl)cyclopropyl]-6-[(3S)-3-methylmorpholin-4-yl]pyrimidin-2-yl]phenyl]urea | 506 | 1.59 |
| 16c | 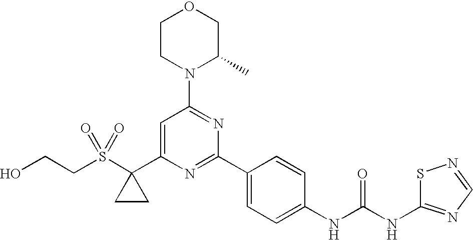 | 1-[4-[4-[1-(2-hydroxyethylsulfonyl)cyclopropyl]-6-[(3S)-3-methylmorpholin-4-yl]pyrimidin-2-yl]phenyl]-3-(1,2,4-thiadiazol-5-yl)urea | 546 | 1.24 |
| 16d | 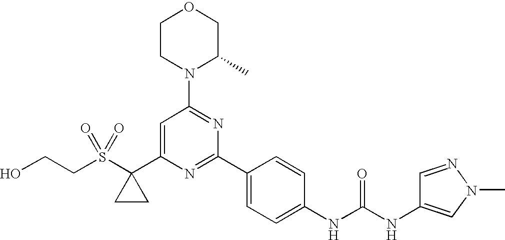 | 1-[4-[4-[1-(2-hydroxyethylsulfonyl)cyclopropyl]-6-[(3S)-3-methylmorpholin-4-yl]pyrimidin-2-yl]phenyl]-3-(1-methylpyrazol-4-yl)urea | 542 | 1.79 |
| 16e | 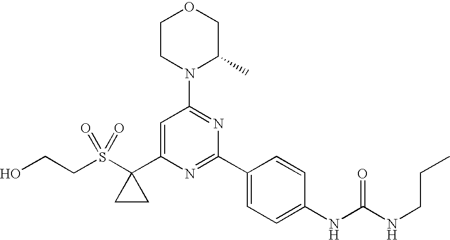 | 3-[4-[4-[1-(2-hydroxyethylsulfonyl)cyclopropyl]-6-[(3S)-3-methylmorpholin-4-yl]pyrimidin-2-yl]phenyl]-1-propylurea | 504 | 1.99 |
| 16f | 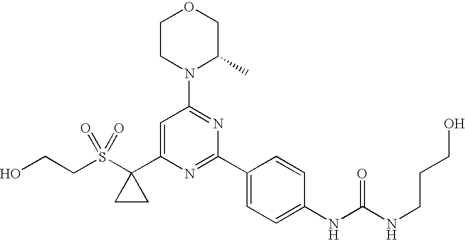 | 1-[4-[4-[1-(2-hydroxyethylsulfonyl)cyclopropyl]-6-[(3S)-3-methylmorpholin-4-yl]pyrimidin-2-yl]phenyl]-3-(3-hydroxypropyl)urea | 520 | 1.61 |
| 16g | 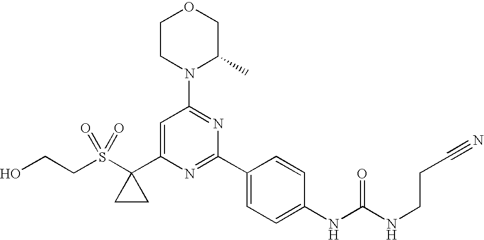 | 3-(2-cyanoethyl)-1-[4-[4-[1-(2-hydroxyethylsulfonyl)cyclopropyl]-6-[(3S)-3-methylmorpholin-4-yl]pyrimidin-2-yl]phenyl]urea | 515 | 1.77 |
| 16h | 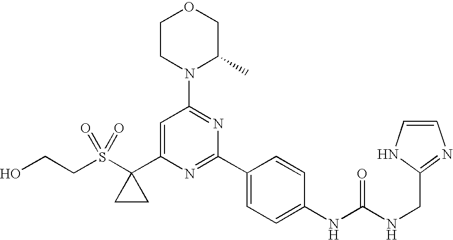 | 1-[4-[4-[1-(2-hydroxyethylsulfonyl)cyclopropyl]-6-[(3S)-3-methylmorpholin-4-yl]pyrimidin-2-yl]phenyl]-3-(1H-imidazol-2-ylmethyl)urea | 542 | 1.64 |
| 16i | 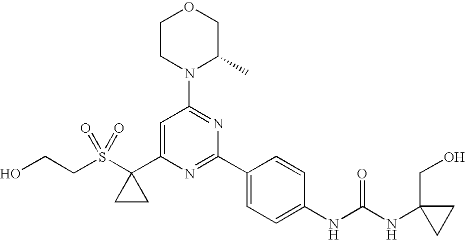 | 1-[4-[4-[1-(2-hydroxyethylsulfonyl)cyclopropyl]-6-[(3S)-3-methylmorpholin-4-yl]pyrimidin-2-yl]phenyl]-3-[1-(hydroxymethyl)cyclopropyl]urea | 532 | 1.66 |

### EXAMPLE 16a

¹H NMR (400 MHz, DMSO-d₆) δ 1.23-1.24(3H, d), 1.53-1.69(7H, m), 1.82-1.92(2H, m), 2.18-2.25(2H, m), 3.17-3.24(1H, td), 3.45-3.52(1H, td), 3.63-3.66(3H, m), 3.75-3.78(1H, d), 3.86-3.91(2H, m), 3.96-3.99(1H, dd), 4.20-4.22(1H, d), 4.56(1H, bs), 5.01-5.04(1H, t), 6.46-6.48(1H, d), 6.78(1H, s), 7.47-7.49(2H, d), 8.19-8.21(2H, d), 8.57(1H, s).

mTOR Kinase Assay (Echo): 0.0101 μM

### EXAMPLE 16b

¹H NMR (400 MHz, DMSO-d₆) δ 1.23-1.24(3H, d), 1.53-1.56(2H, m), 1.64-1.67(2H, m), 3.16-3.24(3H, m), 3.45-3.52(3H, m), 3.62-3.66(3H, m), 3.75-3.78(1H, d), 3.87-3.91(2H, q), 3.96-3.99(1H, dd), 4.19-4.23(1H, d), 4.56(1H, bs), 4.72-4.75(1H, t), 5.01-5.04(1H, t), 6.25-6.27(1H, t), 6.77(1H, s),7.48-7.51(2H, d), 8.20-8.22(2H, d), 8.81(1H, s).

mTOR Kinase Assay (Echo): 0.00577 μM

### EXAMPLE 16c

¹H NMR (400 MHz, DMSO-d₆) δ 1.24-1.26(3H, d), 1.55-1.58(2H, m), 1.66-1.68(2H, m), 3.19-3.26(1H, td), 3.47-3.53(1H, td), 3.63-3.67(3H, m), 3.77-3.79(1H, d), 3.88-3.93(2H, m), 3.97-4.01(1H, dd), 4.22-4.24(1H, d), 4.58(1H, bs), 5.02-5.05(1H, t), 6.83(1H, s), 7.62-7.65(2H, d), 8.31-8.33(2H, d), 8.38(1H, s), 9.43(1H, s), 11.37(1H, bs).

mTOR Kinase Assay (Echo): 0.0016 μM

### EXAMPLE 16d

¹H NMR (400 MHz, DMSO-d₆) δ 1.23-1.25(3H, d), 1.54-1.57(2H, m), 1.65-1.67(2H, m), 3.17-3.25(1H, td), 3.46-3.53(1H, td), 3.62-3.67(3H, m), 3.76-3.79(1H, d), 3.79(3H, s), 3.87-3.92(2H, m), 3.97-4.00(1H, dd), 4.20-4.23(1H, d), 4.56(1H, bs), 5.02-5.05(1H, t), 6.79(1H, s), 7.39-7.40(1H, s), 7.54-7.56(2H, d), 7.76(1H, s), 8.25(2H, d), 8.39(1H, s), 8.84(1H, s).

mTOR Kinase Assay (Echo): 0.00364 μM

### EXAMPLE 16e

¹H NMR (400 MHz, DMSO-d₆) δ 0.87-0.91 (3H, t), 1.23-1.25 (3H, d), 1.42-1.51 (2H, m), 1.53-1.56 (2H, m), 1.64-1.67 (2H, m), 3.05-3.09 (2H, q), 3.17-3.25 (1H, td), 3.46-3.52 (1H, td), 3.62-3.66 (3H, m), 3.75-3.78 (1H, d), 3.87-3.91 (2H, q), 3.96-3.99 (1H, dd), 4.20-4.23 (1H, d), 4.56 (1H, bs), 5.01-5.04 (1H, t), 6.19-6.22 (1H, t), 6.78 (1H, s), 7.49-7.51 (2H, d), 8.19-8.21 (2H, d), 8.65 (1H, s)

### EXAMPLE 16f

¹H NMR (400 MHz, DMSO-d₆) δ 1.23-1.25 (3H, d), 1.53-1.57 (2H, m), 1.59-1.62 (2H, t), 1.64-1.67 (2H, m), 3.15-3.24 (3H, m), 3.46-3.50 (3H, m), 3.63-3.66 (3H, m), 3.75-3.78 (1H, d), 3.87-3.91 (2H, q), 3.96-3.99 (1H, dd), 4.19-4.23 (1H, d), 4.47-4.49 (1H, t), 4.55 (1H, bs), 5.01-5.04 (1H, t), 6.20-6.23 (1H, t), 6.77 (1H, s), 7.49-7.51 (2H, d), 8.19-8.21 (2H, d), 8.72 (1H, s).

### EXAMPLE 16g

¹H NMR (400 MHz, DMSO-d₆) δ 1.23-1.25 (3H, d), 1.54-1.56 (2H, m), 1.65-1.67 (2H, m), 2.69-2.72 (2H, t), 3.17-3.25 (1H, td), 3.35-3.40 (2H, q), 3.46-3.52 (1H, td), 3.62-3.66 (3H, m), 3.75-3.78 (1H, d), 3.87-3.91 (2H, q), 3.96-4.00 (1H, dd), 4.20-4.23 (1H, d), 4.56 (1H, bs), 5.01-5.04 (1H, t), 6.52-6.55 (1H, t), 6.78 (1H, s), 7.51-7.53 (2H, d), 8.21-8.23 (2H, d), 8.93 (1H, s).

### EXAMPLE 16h

¹H NMR (400 MHz, DMSO-d₆) δ 1.23-1.25 (3H, d), 1.54-1.56 (2H, m), 1.64-1.67 (2H, m), 3.17-3.25 (1H, td), 3.46-3.52 (1H, td), 3.62-3.66 (3H, m), 3.75-3.78 (1H, d), 3.87-3.91 (2H, q), 3.96-3.99 (1H, dd), 4.20-4.23 (1H, d), 4.32-4.34 (2H, d), 4.56 (1H, bs), 5.01-5.04 (1H, t), 6.62-6.64 (1H, t), 6.78 (1H, s), 6.93-6.96 (2H, bs), 7.51-7.53 (2H, d), 8.21-8.23 (2H, d), 8.94 (1H, s), 11.84 (1H, s).

### EXAMPLE 16i

¹H NMR (400 MHz, DMSO-d₆) δ 0.64-0.67 (2H, m), 0.70-0.73 (2H, m), 1.23-1.24 (3H, d), 1.53-1.56 (2H, m), 1.64-1.67 (2H, m), 3.17-3.25 (1H, td), 3.44-3.52 (3H, m), 3.62-3.66 (3H, m), 3.75-3.78 (1H, d), 3.87-3.91 (2H, q), 3.96-4.00 (1H, dd), 4.19-4.23 (1H, d), 4.55 (1H, bs), 4.84 (1H, bs), 5.01-5.04 (1H, t), 6.58 (1H, s), 6.78 (1H, s), 7.47-7.49 (2H, d), 8.20-8.22 (2H, d), 8.68 (1H, s).

The preparation of phenyl N-[4-[4-[1-(2-hydroxyethylsulfonyl)cyclopropyl]-6-[(3S)-3-methylmorpholin-4-yl]pyrimidin-2-yl]phenyl]carbamate is described below.

### Phenyl N-[4-[4-[1-(2-hydroxyethylsulfonyl)cyclopropyl]-6-[(3S)-3-methylmorpholin-4-yl]pyrimidin-2-yl]phenyl]carbamate

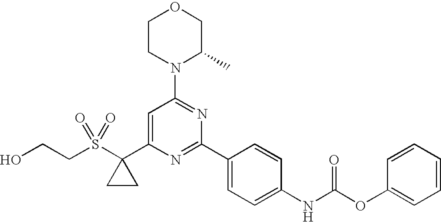

Phenyl chloroformate (0.043 mL, 0.34 mmol) was added to 2-[1-[2-(4-aminophenyl)-6-[(3S)-3-methylmorpholin-4-yl]pyrimidin-4-yl]cyclopropyl]sulfonylethanol (0.144 g, 0.34 mmol) and sodium bicarbonate (0.043 g, 0.52 mmol) in dioxane (20 mL) and the resulting slurry stirred at RT overnight. The reaction mixture was partitioned between DCM and water. The organic layer was dried (MgSO₄), filtered and concentrated in vacuo. The crude product was purified by flash silica chromatography, eluting with 0 to 40% ethyl acetate in DCM, to give the desired material as a beige solid (0.1 17 g).

LCMS Spectrum: m/z (ES+) (M+H)+=539; HPLC tR=2.50 min.

### 2-[1-[2-(4-Aminophenyl)-6-[(3S)-3-methylmorpholin-4-yl1pyrimidin-4-yl]cyclopropyl]sulfonylethanol

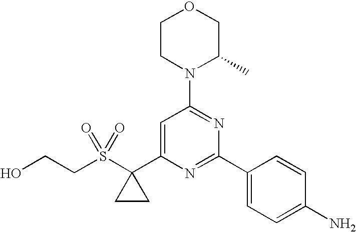

Bis(triphenylphosphine)palladium (II) chloride (0.012 g, 0.02 mmol) was added to 2-[[2-chloro-6-[(3S)-3-methylmorpholin-4-yl]pyrimidin-4-yl]methylsulfonyl]ethoxy-tri(propan-2-yl)silane (0.178 g, 0.34 mmol), 4-(4,4,5,5-tetramethyl-1,3,2-dioxaborolan-2-yl)aniline (0.113 g, 0.52 mmol) and an aqueous solution of sodium carbonate (0.859 mL, 1.72 mmol) in a mixture of solvents (18% DMF, 82% of a 7:3:2 mixture of DME:water:Ethanol). The resulting solution was stirred at 80° C. for 4 hours, the solvent removed and the residue partitioned between DCM and water. The organic layer was separated and tetrabutylammonium fluoride (1.718 mL, 1.72 mmol) added. The reaction was allowed to stir for several hours and then additional tetrabutylammonium fluoride (2 mL) was added and the reaction allowed to stir for 2 days. The mixture was washed with a saturated aqueous solution of ammonium chloride, dried (MgSO₄) filtered and concentrated in vacuo to give the desired material which was used without further purification.

LCMS Spectrum: m/z (ES+) (M+H)+=419; HPLC tR=1.83 min.

### EXAMPLE 17

### 1-[4-[4-[1-(5-Fluoropyridin-3-yl)sulfonylcyclopropyl]-6-[(3S)-3-methylmorpholin-4-yl]pyrimidin-2-yl]phenyl]-3-(2-hydroxyethyl)urea

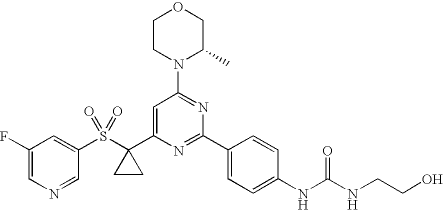

To a solution of phenyl N-[4-[4-[1-(5-fluoropyridin-3-yl)sulfonylcyclopropyl]-6-[(3S)-3-methylmorpholin-4-yl]pyrimidin-2-yl]phenyl]carbamate (140 mg, 0.24 mmol) in DMF (2 mL) was added triethylamine (0.099 mL, 0.71 mmol) followed by ethanolamine (72mg, 1.19 mmol) and the reaction heated at 50° C. for 2 hours. The crude product was purified by preparative HPLC, using decreasingly polar mixtures of water (containing 1% NH3) and MeCN as eluents, to give the desired material as a white solid (55 mg).

NMR Spectrum: ¹H NMR (400.132 MHz, DMSO-d₆) δ 1.20 (3H, d), 1.64-1.67 (2H, m), 1.99-2.02 (2H, m), 3.15-3.20 (3H, m), 3.44-3.49 (2H, m), 3.61 (1H, d), 3.96 (1H, dd), 4.18 (1H, d), 4.50 (1H, s), 4.73 (1H, t), 6.29 (1H, t), 6.70 (1H, s), 7.38 (2H, d), 7.74 (2H, d), 8.18-8.21 (1H, m), 8.81 (2H, d), 8.95 (1H, d)

LCMS Spectrum: m/z (ESI+)(M+H)+=557; HPLC tR=1.82 min.

mTOR Kinase Assay (Echo): 0.00116 μM

The compounds below were prepared in an analogous fashion from phenyl N-[4-[4-[1-(5-fluoropyridin-3-yl)sulfonylcyclopropyl]-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 17a | 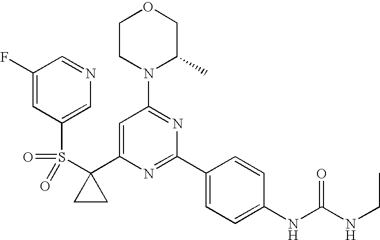 | 3-ethyl-1-[4-[4-[1-(5-fluoropyridin-3-yl)sulfonylcyclopropyl]-6-[(3S)-3-methylmorpholin-4-yl]pyrimidin-2-yl]phenyl]urea | 541 | 1.78 |
| 17b | 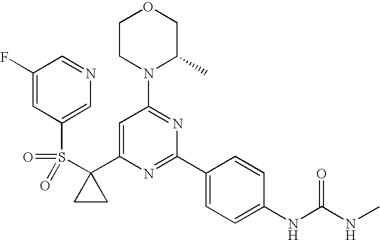 | 1-[4-[4-[1-(5-fluoropyridin-3-yl)sulfonylcyclopropyl]-6-[(3S)-3-methylmorpholin-4-yl]pyrimidin-2-yl]phenyl]-3-methylurea | 527 | 1.99 |
| 17c | 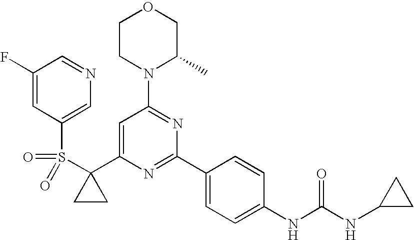 | 3-cyclopropyl-1-[4-[4-[1-(5-fluoropyridin-3-yl)sulfonylcyclopropyl]-6-[(3S)-3-methylmorpholin-4-yl]pyrimidin-2-yl]phenyl]urea | 553 | 2.13 |

### EXAMPLE 17a

¹H NMR (400.132 MHz, DMSO-d₆) δ 1.07 (3H, t), 1.20 (3H, d), 1.64-1.67 (2H, m), 1.99-2.02 (2H, m), 3.09-3.21 (3H, m), 3.47 (1H, dt), 3.61 (1H, d), 3.96 (1H, dd), 4.18 (1H, d), 4.50 (1H, s), 6.19 (1H, t), 6.70 (1H, s), 7.38 (2H, d), 7.74 (2H, d), 8.18-8.19 (1H, m), 8.20-8.21 (1H, m), 8.68 (1H, s), 8.81 (1H, s), 8.95 (1H, d),

mTOR Kinase Assay (Echo): 0.000942 μM

### EXAMPLE 17b

¹H NMR (400.132 MHz, DMSO-d₆) δ 1.20 (3H, d), 1.64-1.67 (2H, m), 1.99-2.02 (2H, m), 2.66 (3H, d), 3.14-3.21 (1H, m), 3.47 (1H, dt), 3.61 (1H, dd), 3.75 (1H, d), 3.96 (1H, dd), 4.18 (1H, d), 4.50 (1H, s), 6.09-6.12 (1H, m), 6.70 (1H, s), 7.39 (2H, d), 7.74 (2H, d), 8.18-8.22 (1H, m), 8.79 (2H, d), 8.94 (1H, d).

mTOR Kinase Assay (Echo): 0.000584 μM

### EXAMPLE 17c

¹H NMR (400.132 MHz, DMSO-d₆) δ 0.41-0.44 (2H, m), 0.60-0.67 (2H, m), 1.20 (3H, d), 1.65-1.66 (2H, m), 1.99-2.02 (2H, m), 3.15-3.21 (2H, m), 3.42-3.49 (1H, m), 3.61 (1H, d), 3.75 (1H, d), 3.96 (1H, d), 4.18 (1H, d), 4.50 (1H, s), 6.60 (1H, s), 6.70 (1H, s), 7.40 (2H, d), 7.74 (2H, d), 8.18-8.22 (1H, m), 8.69 (1H, s), 8.81 (1H, s), 8.95 (1H, d).

mTOR Kinase Assay (Echo): 0.00179 μM

The preparation of phenyl N-[4-[4-[1-(5-fluoropyridin-3-yl)sulfonylcyclopropyl]-6-[(3S)-3-methylmorpholin-4-yl]pyrimidin-2-yl]phenyl]carbamate is described below.

### Phenyl N-[4-[4-[1-(5-fluoropyridin-3-yl)sulfonylcyclopropyl]-6-[(3S)-3-methylmorpholin-4-yl]pyrimidin-2-yl]phenyl]carbamate

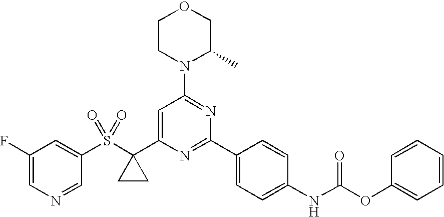

To a solution of 4-[4-[1-(5-fluoropyridin-3-yl)sulfonylcyclopropyl]-6-[(3S)-3-methylmorpholin-4-yl]pyrimidin-2-yl]aniline (220 mg, 0.47 mmol) in 1,4-dioxane (5 mL) was added sodium bicarbonate (59 mg, 0.70 mmol) and phenyl chloroformate (0.059 mL, 0.47 mmol) and the reaction stirred at RT for 2 hours. The reaction mixture was diluted with DCM (10 mL), and washed with water (10 mL), the organic layer dried (MgSO₄), filtered and evaporated. The crude solid was triturated with diethyl ether to give a solid which was collected by filtration and dried under vacuum to give the desired product as a white solid (280 mg).

NMR Spectrum: ¹H NMR (400.132 MHz, DMSO-d₆) δ 1.21 (3H, d), 1.65-1.69 (2H, m), 2.00-2.03 (2H, m), 3.15-3.22 (1H, m), 3.44-3.52 (1H, m), 3.62 (1H, dd), 3.75 (1H, d), 3.96 (1H, dd), 4.21 (1H, d), 4.51 (1H, s), 6.75 (1H, s), 7.24-7.27 (3H, m), 7.45 (2H, t), 7.53 (2H, d), 7.85 (2H, d), 8.20-8.23 (1H, m), 8.81 (1H, s), 8.94 (1H, d), 10.40 (1H, s)

LCMS Spectrum: m/z (ESI+) (M+H)+=590; HPLC tR=2.96 min.

### 4-[4-l [-(5-Fluoropyridin-3-yl)sulfonylcyclopropyl]-6-[(3S)-3-methylmorpholin-4-yl]pyrimidin-2-yl]aniline

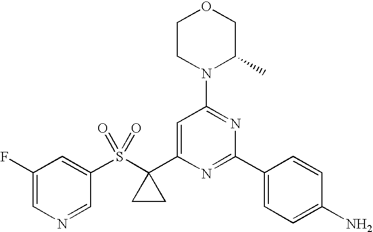

To a solution of 2-chloro-4-[1-(5-fluoropyridin-3-yl)sulfonylcyclopropyl]-6-[(3S)-3-methylmorpholin-4-yl]pyrimidine (700 mg, 1.70 mmol) in DMF (0.48 mL), DME (9.33 mL), water (4.0 mL) and ethanol (2.67 mL) was added 4-(4,4,5,5-tetramethyl-1,3,2-dioxaborolan-2-yl)aniline (483mg, 2.2 mmol), sodium carbonate (2.5 mL, 5.09 mmol) and dichlorobis(triphenylphosphine)palladium(II) (59.5 mg, 0.08 mmol) and the suspension heated at 95° C. for 2 hours. The reaction mixture was cooled to RT, diluted with ethyl acetate (10 mL) and washed with water (2×10 mL). The organic layer was dried (MgSO₄), filtered and evaporated. The crude product was purified by flash silica chromatography, elution gradient 5 to 60% ethyl acetate in isohexane, to give the desired material as a cream solid (160 mg).

NMR Spectrum: ¹H NMR (400.132 MHz, DMSO-d₆) δ 1.19 (3H, d), 1.61-1.65 (2H, m), 1.96-2.00 (2H, m), 3.11-3.18 (1H, m), 3.42-3.49 (1H, m), 3.60 (1H, dd), 3.74 (1H, d), 3.95 (1H, dd), 4.14 (1H, d), 4.46 (1H, s), 5.53 (2H, s), 6.49 (2H, d), 6.60 (1H, s), 7.57 (2H, d), 8.16-8.20 (1H, m), 8.80-8.80 (1H, m), 8.93 (1H, d)

LCMS Spectrum: m/z (ESI+) (M+H)+=470; HPLC tR=2.30 min.

### 2-Chloro-4-[1-(5-fluoropyridin-3-yl)sulfonylcyclopropyl]-6-[(3S)-3-methylmorpholin-4-yl]pyrimidine

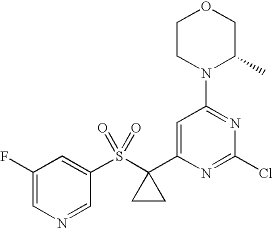

2-Chloro-4-[(5-fluoropyridin-3-yl)sulfonylmethyl] -6-[(3S)-3-methylmorpholin-4-yl]pyrimidine (950 mg, 2.40 mmol) was dissolved in toluene (15 mL) and 10N sodium hydroxide solution (2.45 mL, 24.5 mmol) added, followed by 1,2-dibromoethane (0.42 mL, 4.91 mmol). The reaction was stirred at 60° C. for 3 hours. The reaction mixture was evaporated to dryness, redissolved in ethyl acetate (200 mL), and washed sequentially with water (200 mL) and saturated brine solution (100 mL). The organic layer was dried (MgSO₄), filtered and evaporated to afford crude product. The crude product was purified by flash silica chromatography, elution gradient 0 to 50% ethyl acetate in DCM, to give the desired product as a white solid (700 mg,).

NMR Spectrum: ¹H NMR (400.132 MHz, DMSO-d₆) δ 1.17 (3H, d), 1.61-1.63 (2H, m), 1.94-1.97 (2H, m), 3.16 (1H, dt), 3.40 (1H, dt), 3.55 (1H, dd), 3.70 (1H, d), 3.91 (1H, dd), 4.00 (1H, s), 4.33 (1H, s), 6.78 (1H, s), 8.21 (1H, dt), 8.79 (1H, t), 8.96 (1H, d)

LCMS Spectrum: m/z (ESI+)(M+H)+ 413, HPLC tR=2.14 min

### 2-Chloro-4-[(5-fluoropyridin-3-yl)sulfonylmethyl]-6-[(3S)-3-methylmorpholin-4-yl]pyrimidine

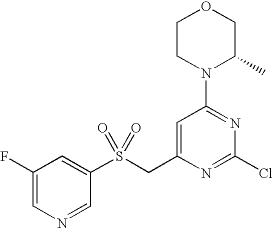

3-Chloroperoxybenzoic acid (2.107 g, 9.16 mmol) was added portion-wise to 2-chloro-4-[(5-fluoropyridin-3-yl)sulfanylmethyl]-6-[(3S)-3-methylmorpholin-4-yl]pyrimidine (1.3 g, 3.66 mmol), in DCM (18.32 mL) and the reaction stirred at RT for 2 hours. The reaction mixture was washed with a saturated aqueous solution of sodium hydrogen carbonate (50 mL) and the organic layer dried (MgSO₄), filtered and evaporated to afford crude product. The crude product was purified by flash silica chromatography, elution gradient 0 to 60% ethyl acetate in DCM, to give the desired material as a white solid (0.940 g).

NMR Spectrum: ¹H NMR (400.132 MHz, DMSO-d₆) δ 1.19 (3H, d), 3.16-3.23 (1H, m), 3.44 (1H, dt), 3.59 (1H, dd), 3.72 (1H, d), 3.93 (1H, dd), 3.97 (1H, s), 4.22 (1H, s), 4.84 (2H, s), 6.84 (1H, s), 8.21 (1H, dt), 8.80 (1H, t), 8.99 (1H, d)

LCMS Spectrum: m/z (ESI+)(M+H)+ 387, HPLC tR=1.86 min

### 2-Chloro-4-[(5-fluoropyridin-3-yl)sulfanylmethyl]-6-[(3S)-3-methylmorpholin-4-yl]pyrimidine

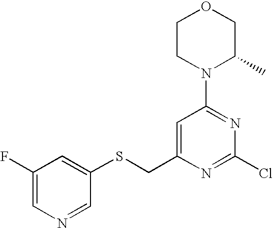

Potassium hydroxide (1.235 g, 22.01 mmol) was added to (5-fluoropyridin-3-yl)dimethylaminomethanedithioate (1.19 g, 5.50 mmol) in ethanol (27.5 mL) at RT. The resulting solution was heated at 65° C. for 4 hours The reaction was cooled and 2-chloro-4-(iodomethyl)-6-[(3S)-3-methylmorpholin-4-yl]pyrimidine (2.72 g, 7.70 mmol) added. The reaction mixture was then stirred at RT for 4 hours. Water (50 mL) was added and the reaction mixture extracted with DCM (2×100 mL). The combined organics were dried (MgSO₄), filtered and concentrated to give crude product. The crude product was purified by flash silica chromatography, elution gradient 0 to 50% ethyl acetate in DCM, to give the desired material as a white waxy solid (1.5 g).

NMR Spectrum: ¹H NMR (400.132 MHz, DMSO-d₆) δ 1.14 (3H, d), 3.11-3.18 (1H, m), 3.37-3.44 (1H, m), 3.56 (1H, dd), 3.70 (1H, d), 3.91 (1H, dd), 3.95 (1H, s), 4.21 (3H, $mult$), 6.81 (1H, s), 7.89 (1H, dt), 8.40-8.42 (2H, m)

LCMS Spectrum: m/z (ESI+)(M+H)+ 355, HPLC tR=2.12 min

### (5-Fluoropyridin-3-yl) dimethylaminomethanedithioate

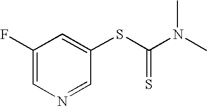

3-Bromo-5-fluoropyridine (2.26g, 12.84 mmol) was added portion-wise to isopropylmagnesium chloride-lithium chloride complex (14% in THF, 13.32 mL, 12.84 mmol) at 0° C. over a period of 2 minutes under a nitrogen atmosphere. The resulting solution was warmed to RT over a period of 2 hours then cooled to 0° C. and tetramethylthiuram disulfide (3.09 g, 12.84 mmol) in DCM (12.84 mL) added. The reaction was warmed to RT and stirred for 3 hours. The reaction was poured into a saturated aqueous solution of ammonium chloroid (50 mL) and the aqueous layer extracted with DCM (2×100 mL). The combined organic layers were dried (MgSO₄), filtered and concentrated in vacuo. The crude product was purified by flash silica chromatography, elution gradient 0 to 60% ethyl acetate in DCM, to give the desired material as a cream waxy solid (1.69 g).

NMR Spectrum: ¹H NMR (400.132 MHz, DMSO-d₆) δ 3.48 (3H, s), 3.52 (3H, s), 7.89 (1H, ddd), 8.41 (1H, t), 8.71 (1H, d)

LCMS Spectrum: m/z (ESI+)(M+H)+ 217, HPLC tR=1.80 min

The preparation of 2-chloro-4-(iodomethyl)-6-[(3S)-3-methylmorpholin-4-yl]pyrimidine was described earlier.

### EXAMPLE 18

### 1-[4-[4-(1-tert-Butylsulfonylcyclopropyl)-6-[(3S)-3-methylmorpholin-4-yl]pyrimidin-2-yl]phenyl]-3-cyclopropylurea

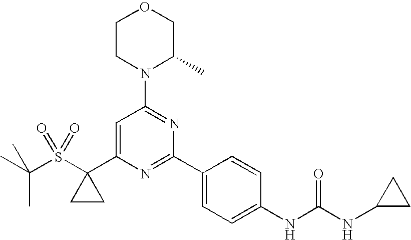

Cyclopropylamine (57 mg, 1.0 mmol) was added to phenyl N-[4-[4-(1-tert-butylsulfonylcyclopropyl)-6-[(3S)-3-methylmorpholin-4-yl]pyrimidin-2-yl]phenyl]carbamate (120 mg, 0.22 mmol) and triethylamine (0.20 mL, 1.4 mmol) in DMF (1 mL). The reaction mixture was allowed to stand at RT for 4 days. The crude product was purified by preparative HPLC, using decreasingly polar mixtures of water (containing 1% ammonia) and acetonitrile as eluents, to afford the desired material as a colourless solid (63 mg).

NMR Spectrum: ¹H NMR (399.9 MHz, DMSO-d₆) δ 0.40-0.44 (2H, m), 0.63-0.68 (2H, m), 1.21-1.27 (12H, m), 1.47-1.53 (1H, m), 1.62-1.69 (3H, m), 2.54-2.60 (1H, m), 3.15-3.24 (1H, m), 3.46-4.54 (1H, m), 3.63-3.67 (1H, m), 3.77 (1H, d), 3.95-4.02 (1H, d), 4.18 (1H, d), 4.43 (1H, br, s), 6.44 (1H, d), 6.97 (1H, s), 7.51 (2H, d), 8.23 (2H, d), 8.56 (1H, s),

LCMS Spectrum: m/z (ESI+)(M+H)+=514; HPLC tR=2.39 min.

mTOR Kinase Assay (Echo): 0.00135 μM

The following compounds were made in an analogous fashion from phenyl N-[4-[4-(1-tert-butylsulfonylcyclopropyl)-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 18a | 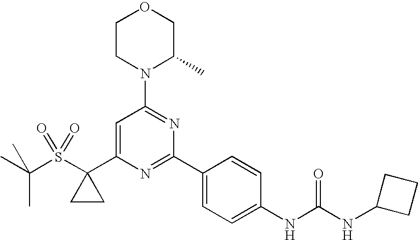 | 1-[4-[4-(1-tert-butylsulfonylcyclopropyl)-6-[(3S)-3-methylmorpholin-4-yl]pyrimidin-2-yl]phenyl]-3-cyclobutylurea | 528 | 2.62 |
| 18b | 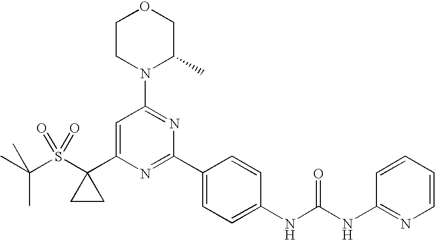 | 1-[4-[4-(1-tert-butylsulfonylcyclopropyl)-6-[(3S)-3-methylmorpholin-4-yl]pyrimidin-2-yl]phenyl]-3-pyridin-2-ylurea | 551 | 2.83 |
| 18c | 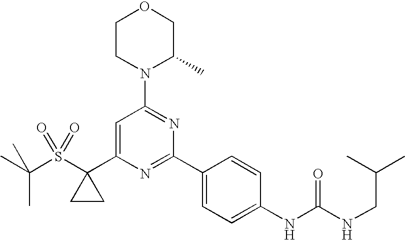 | 1-[4-[4-(1-tert-butylsulfonylcyclopropyl)-6-[(3S)-3-methylmorpholin-4-yl]pyrimidin-2-yl]phenyl]-3-(2-methylpropyl)urea | 530 | 2.72 |
| 18d | 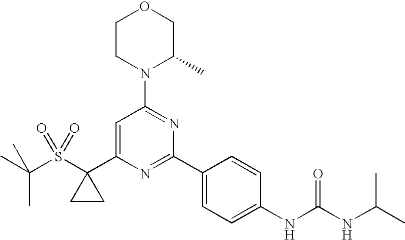 | 1-[4-[4-(1-tert-butylsulfonylcyclopropyl)-6-[(3S)-3-methylmorpholin-4-yl]pyrimidin-2-yl]phenyl]-3-propan-2-ylurea | 516 | 2.54 |
| 18e | 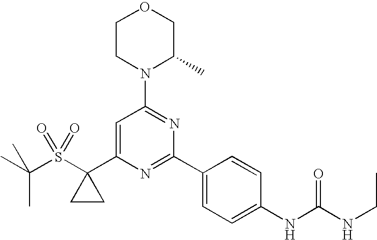 | 1-[4-[4-(1-tert-butylsulfonylcyclopropyl)-6-[(3S)-3-methylmorpholin-4-yl]pyrimidin-2-yl]phenyl]-3-ethylurea | 502 | 2.38 |
| 18f | 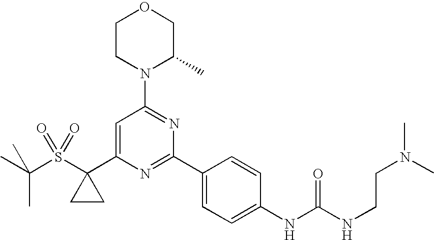 | 1-[4-[4-(1-tert-butylsulfonylcyclopropyl)-6-[(3S)-3-methylmorpholin-4-yl]pyrimidin-2-yl]phenyl]-3-(2-dimethylaminoethyl)urea | 545 | 2.33 |
| 18g | 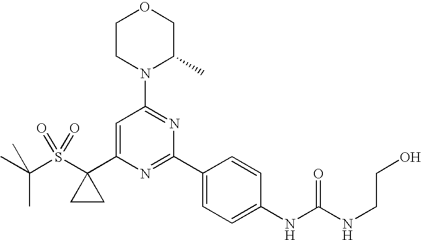 | 1-[4-[4-(1-tert-butylsulfonylcyclopropyl)-6-[(3S)-3-methylmorpholin-4-yl]pyrimidin-2-yl]phenyl]-3-(2-hydroxyethyl)urea | 518 | 1.98 |
| 18h | 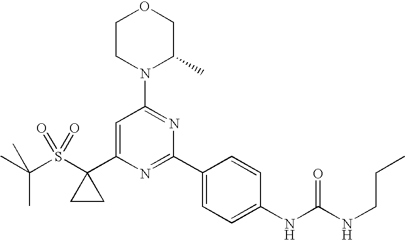 | 1-[4-[4-(1-tert-butylsulfonylcyclopropyl)-6-[(3S)-3-methylmorpholin-4-yl]pyrimidin-2-yl]phenyl]-3-propylurea | 516 | 2.56 |
| 18i | 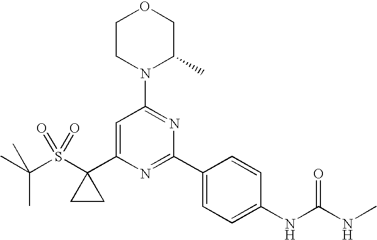 | 1-[4-[4-(1-tert-butylsulfonylcyclopropyl)-6-[(3S)-3-methylmorpholin-4-yl]pyrimidin-2-yl]phenyl]-3-methylurea | 488 | 2.2 |
| 18j | 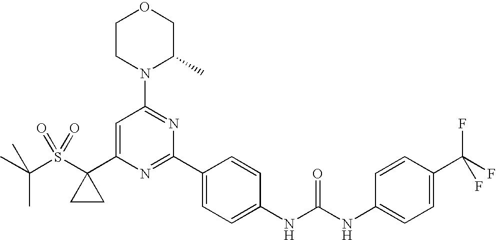 | 1-[4-[4-(1-tert-butylsulfonylcyclopropyl)-6-[(3S)-3-methylmorpholin-4-yl]pyrimidin-2-yl]phenyl]-3-[4-(trifluoromethyl)phenyl]urea | 618 | 3.29 |
| 18k | 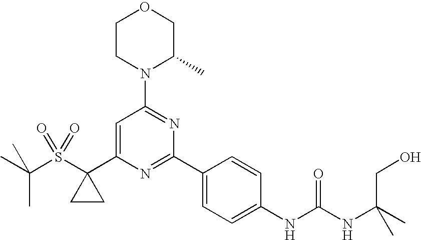 | 1-[4-[4-(1-tert-butylsulfonylcyclopropyl)-6-[(3S)-3-methylmorpholin-4-yl]pyrimidin-2-yl]phenyl]-3-(1-hydroxy-2-methylpropan-2-yl)urea | 546 | 2.32 |
| 18l | 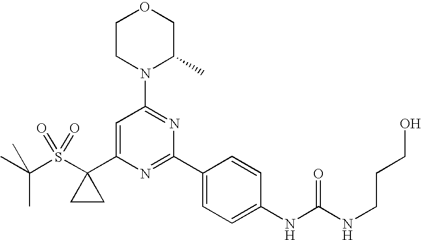 | 1-[4-[4-(1-tert-butylsulfonylcyclopropyl)-6-[(3S)-3-methylmorpholin-4-yl]pyrimidin-2-yl]phenyl]-3-(3-hydroxypropyl)urea | 532 | 2.05 |
| 18m | 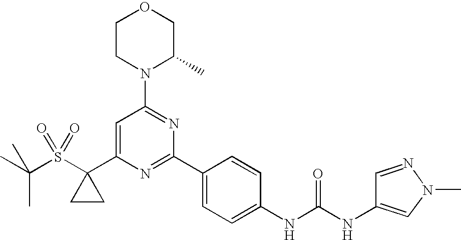 | 1-[4-[4-(1-tert-butylsulfonylcyclopropyl)-6-[(3S)-3-methylmorpholin-4-yl]pyrimidin-2-yl]phenyl]-3-(1-methylpyrazol-4-yl)urea | 554 | 2.22 |

### EXAMPLE 18a

¹H NMR (399.9 MHz, DMSO-d₆) δ 1.21-1.27 (12H, m), 1.47-1.53 (1H, m), 1.59-1.68 (3H, m), 1.81-1.91 (2H, m), 2.17-2.26 (2H, m), 3.15-3.23 (1H, m), 3.45-3.54 (1H, m), 3.62-3.68 (1H, m), 3.77 (1H, d), 3.95-4.02 (1H, m), 4.10-4.42 (2H, m), 4.42 (1H, br, s), 6.47 (1H, d), 6.97 (1H, s), 7.48 (2H, d), 8.22 (2H, d), 8.57 (1H, s).

mTOR Kinase Assay (Echo): 0.00967 μM

### EXAMPLE 18b

¹H NMR (399.9 MHz, DMSO-d₆) δ 1.25 (12H, m), 1.49-1.54 (1H, m), 1.63-1.73 (3H, m), 3.16-3.25 (1H, m), 3.48-3.54 (1H, m), 3.64-3.68 (1H, m), 3.78 (1H, d), 3.98-4.01 (1H, m), 4.20 (1H, d), 4.43 (1H, br, s), 7.00 (1H, s), 7.03-7.06 (1H, m), 7.58 (1H, d), 7.65 (2H, d), 7.78 (1H, t), 8.29-8.34 (3H, m), 9.45 (1H, s), 10.57 (1H, s).

mTOR Kinase Assay (Echo): 0.0147 μM

### EXAMPLE 18c

¹H NMR (399.9 MHz, DMSO-d₆) δ 0.90 (6H, d), 1.22-1.25 (12H, m), 1.47-1.53 (1H, m), 1.62-1.75 (4H, m), 2.95 (2H, t), 3.16-3.23 (1H, m), 3.47-3.53 (1H, m), 3.63-3.67 (1H, m), 3.77 (1H, d), 3.96-4.00 (1H, m), 4.18 (1H, d), 4.42 (1H, br, s), 6.25 (1H, t), 6.97 (1H, s), 7.50 (2H, d), 8.23 (2H, d), 8.66 (1H, s).

mTOR Kinase Assay (Echo): 0.032 μM

### EXAMPLE 18d

¹H NMR (399.9 MHz, DMSO-d₆) δ 1.12 (6H, d), 1.21-1.26 (12H, m), 1.45-1.53 (1H, m), 1.62-1.71 (3H, m), 3.13-3.25 (1H, m), 3.44-3.53 (1H, m), 3.62-3.67 (1H, m), 3.73-3.82 (2H, m), 3.95-4.02 (1H, m), 4.18 (1H, d), 4.42 (1H, br, s), 6.07 (1H, d), 6.97 (1H, s), 7.48 (2H, d), 8.23 (2H, d), 8.55 (1H, s).

mTOR Kinase Assay (Echo): 0.0151 μM

### EXAMPLE 18e

¹H NMR (399.9 MHz, DMSO-d₆) δ 1.07 (3H, t), 1.23 (3H, d), 1.25 (9H, s), 1.44-1.53 (1H, m), 1.60-1.71 (3H, m), 3.08-3.25 (3H, m), 3.49 (1H, t), 3.65 (1H, d), 3.77 (1H, d), 3.96-3.99 (1H, m), 4.18 (1H, d), 4.43 (1H, br, s), 6.17 (1H, t), 6.97 (1H, s), 7.50 (2H, d), 8.23 (2H, d), 8.68 (1H, s).

mTOR Kinase Assay (Echo): 0.000654 μM

### EXAMPLE 18f

¹H NMR (399.9 MHz, DMSO-d₆) δ 1.20-1.28 (12H, m), 1.47-1.54 (1H, m), 1.62-1.69 (3H, m), 2.19 (6H, s), 2.34 (2H, t), 3.14-3.24 (3H, m), 3.45-3,53 (1H, m), 3.62-3.67 (1H, m), 3.77 (1H, d), 3.95-4.00 (1H, m), 4.18 (1H, d), 4.42 (1H, br, s), 6.17 (1H, t), 6.97 (1H, s), 7.49 (2H, d), 8.23 (2H, d), 8.91 (1H, s).

mTOR Kinase Assay (Echo): 0.12 μM

### EXAMPLE 18g

¹H NMR (399.9 MHz, DMSO-d₆) δ 1.22-1.25 (12H, m), 1.48-1.52 (1H, m), 1.62-1.69 (3H, m), 3.16-3.23 (3H, m), 3.45-3.53 (3H, m), 3.63-3.67 (1H, m), 3.77 (1H, d), 3.97-4.00 (1H, m), 4.18 (1H, d), 4.43 (1H, br, s), 4.74 (1H, t), 6.26 (1H, t), 6.97 (1H, s), 7.49 (2H, d), 8.23 (2H, d), 8.82 (1H, s).

mTOR Kinase Assay (Echo): 0.00123 μM

### EXAMPLE 18h

¹H NMR (399.9 MHz, DMSO-d₆) δ 0.90 (3H, t), 1.19-1.28 (12H, m), 1.42-1.54 (3H, m), 1.61-1.69 (3H, m), 3.07 (2H, q), 3.13-3.23 (1H, m), 3.45-3.53 (1H, m), 3.61-3.68 (1H, m), 3.77 (1H, d), 3.94-4.01 (1H, m), 4.18 (1H, d), 4.42 (1H, br, s), 6.21 (1H, t), 6.97 (1H, s), 7.50 (2H, d), 8.23 (2H, d), 8.67 (1H, s).

mTOR Kinase Assay (Echo): 0.00664 μM

### EXAMPLE 18i

¹H NMR (399.9 MHz, DMSO-d₆) δ 1.22 (3H, d), 1.25 (9H, s), 1.45-1.55 (1H, m), 1.60-1.69 (3H, m), 2.67 (3H, d), 3.14-3.23 (1H, m), 3.50 (1H, t), 3.65 (1H, d), 3.77 (1H, d), 3.94-3.99 (1H, m), 4.18 (1H, d), 4.43 (1H, br, s), 6.08 (1H, d), 6.97 (1H, s), 7.51 (2H, d), 8.23 (2H, d), 8.76 (1H, s).

mTOR Kinase Assay (Echo): 0.00555 μM

### EXAMPLE 18j

¹H NMR (399.9 MHz, CDCl₃) δ 1.31 (3H, d), 1.35 (9H, s), 1.50-1.71 (2H, m), 1.81-1.91 (2H, m), 3.23-3.33 (1H, m), 3.53-3.62 (1H, m), 3.68-3.74 (1H, m), 3.81 (1H, d), 3.98-4.05 (1H, m), 4.17 (1H, d), 4.42 (1H, br, s), 7.03 (1H, s), 7.23 (1H, s), 7.28 (1H, s), 7.48 (2H, d), 7.52-7.55 (4H, m), 8.38 (2H, d).

mTOR Kinase Assay (Echo): 0.0303 μM

### EXAMPLE 18k

¹H NMR (399.9 MHz, DMSO-d₆) δ 1.20-1.27 (18H, m), 1.48-1.52 (1H, m), 1.62-1.69 (3H, m), 3.14-3.23 (1H, m), 3.40 (2H, d), 3.45-3.53 (1H, m), 3.63-3.67 (1H, m), 3.77 (1H, d), 3.95-4.01 (1H, m), 4.18 (1H, d), 4.41 (1H, br, s), 4.96 (1H, s), 6.97 (1H, s), 7.46 (2H, d), 8.22 (2H, d), 8.75 (1H, s).

mTOR Kinase Assay (Echo): 0.0113 μM

### EXAMPLE 18l

¹H NMR (399.9 MHz, DMSO-d₆) δ 1.22-1.25 (12H, m), 1.48-1.52 (1H, m), 1.57-1.68 (5H, m), 3.15-3.23 (3H, m), 3.43-3.54 (3H, m), 3.63-3.67 (1H, m), 3.77 (1H, d), 3.96-4.00 (1H, m), 4.18 (1H, d), 4.43 (1H, br, s), 4.49 (1H, t), 6.21 (1H, t), 6.97 (1H, s), 7.50 (2H, d), 8.23 (2H, d), 8.73 (1H, s).

mTOR Kinase Assay (Echo): 0.0102 μM

### EXAMPLE 18m

¹H NMR (399.9 MHz, DMSO-d₆) δ 1.21-1.29 (12H, m), 1.48-1.55 (1H, m), m), 1.61-1.71 (3H, m), 3.16-3.25 (1H, m), 3.49 (1H, t), 3.66 (1H, d), 3.74-3.83 (4H, m), 3.99 (1H, d), 4.19 (1H, d), 4.43 (1H, br, s), 6.99 (1H, s), 7.39 (1H, s), 7.55 (2H, d), 7.77 (1H, s), 8.27 (2H, d), 8.39 (1H, s), 8.85 (1H, s).

mTOR Kinase Assay (Echo): 0.00315 μM

The preparation of phenyl N-[4-[4-(1-tert-butylsulfonylcyclopropyl)-6-[(3S)-3-methylmorpholin-4-yl]pyrimidin-2-yl]phenyl]carbamate is described below.

### Phenyl N-[4-[4-(1-tert-butylsulfonylcyclopropyl)-6-[(3S)-3-methylmorpholin-4-yl]pyrimidin-2-yl]phenyl carbamate

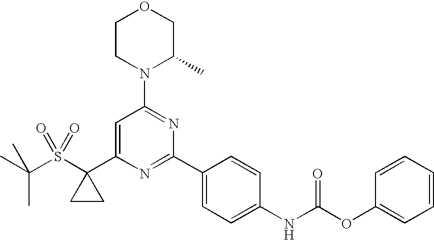

Phenyl chloroformate (0.809 mL, 6.44 mmol) was added to 4-[4-(1-tert-butylsulfonylcyclopropyl)-6-[(3S)-3-methylmorpholin-4-yl]pyrimidin-2-yl]aniline (2.52 g, 5.85 mmol) and sodium hydrogen carbonate (0.738 g, 8.78 mmol) in dioxane (45 mL) at RT. The mixture was stirred at RT for 2 hours. The mixture was partitioned between ethyl acetate and water. The organic layer was washed with brine, dried (MgSO₄) and concentrated under reduced pressure. The residue was purified by chromatography on silica, eluting with 10% -100% ethyl acetate in isohexane, to give the desired material as a near colourless solid (2.99 g).

NMR Spectrum: ¹H NMR (399.9 MHz, CDCl₃) δ 1.31-1.34 (12H, m), 1.48-4.58 (1H, m), 1.62-1.70 (1H, m), 1.80-1.89 (2H, m), 3.31 (1H, dt), 3.60 (1H, dt), 3.75 (1H, dd), 3.82 (1H, d), 4.04 (1H, dd), 4.20 (1H, d), 4.45 (1H, br), 7.06-7.11 (2H, m), 7.19-7.28 (3H, m), 7.41 (2H, t), 7.54 (2H, d), 8.40 (2H, d)

LCMS Spectrum: m/z (ESI+)(M+H)+=551; HPLC tR=3.06 min.

### 4-[4-(1-tert-Butylsulfonylcyclopropyl)-6-[(3S)-3-methylmorpholin-4-yl]pyrimidin-2-yl]aniline

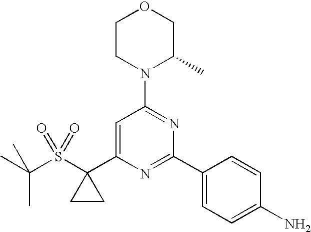

Dichlorobis(triphenylphosphine)-palladium(II) (0.185 g, 0.26 mmol) was added to 4-(1-tert-butylsulfonylcyclopropyl)-2-chloro-6-[(3S)-3-methylmorpholin-4-yl]pyrimidine (1.97 g, 5.27 mmol), 4-(4,4,5,5-tetramethyl-1,3,2-dioxaborolan-2-yl)aniline (1.501 g, 6.85 mmol) and 2M aqueous sodium carbonate (9.48 mL, 18.97 mmol) in DMF (11 mL), DME (11 mL), ethanol (11 mL) and water (27.5 mL) at RT under nitrogen. The reaction was purged with nitrogen for 15 minutes and the resulting mixture was stirred at 80° C. for 16 hours. The reaction mixture was partitioned between ethyl acetate and water. The organic solution was dried (MgSO₄) and 25 concentrated under reduced pressure. The residue was purified by chromatography on silica, eluting with 25% -100% ethyl acetate in isohexane, to give the desired material as a yellow solid (2.24 g).

NMR Spectrum: ¹H NMR (399.9 MHz, CDCl₃) δ 1.31 (3H, d), 1.32 (9H, s), 1.44-1.55 (1H, m), 1.60-1.68 (1H, m), 1.77-1.86 (2H, m), 3.28 (1H, dt), 3.59 (1H, dt), 3.74 (1H, dd), 3.81 (1H, d), 3.90 (2H, s), 4.03 (1H, dd), 4.18 (1H, d), 4.44 (1H, br), 6.71 (2H, d), 6.99 (1H, s), 8.24 (2H, d).

LCMS Spectrum: m/z (ESI+)(M+H)+=431; HPLC tR=2.43 min.

### 4-(1-tert-Butylsulfonylcyclopropyl)-2-chloro-6-[(3S)-3-methylmorpholin-4-yl]pyrimidine

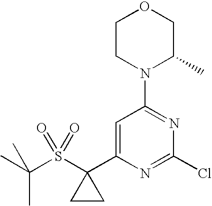

1,2-Dibromoethane (0.349 mL, 15.40 mmol) was added to 4-(tert-butylsulfonylmethyl)-2-chloro-6-[(3S)-3-methylmorpholin-4-yl]pyrimidine (2.68 g, 7.7 mmol) in toluene (40 mL) followed by tetrabutylammonium bromide (0.248 g, 0.77 mmol) and sodium hydroxide concentrate (7.70 mL, 77 mmol). The reaction mixture was vigorously stirred and heated at 60° C. for 1 hour. The reaction mixture was cooled and diluted with ethyl acetate and washed with water. The organic solution was concentrated under reduced pressure. The residue was purified by chromatography on silica, eluting with 0 to 50% ethyl acetate in DCM, to give the desired material as a colourless solid (1.97 g).

NMR Spectrum: ¹H NMR (399.9 MHz, CDCl₃) δ 1.20-1.35 (12H, m), 1.46-1.50 (1H, m), 1.52-1.61 (1H, m), 1.77-1.86 (2H, m), 3.26 (1H, dt), 3.54 (1H, dt), 3.68 (1H, dd), 3.78 (1H, d), 3.98-4.08 (2H, m), 4.29 (1H, br, s), 7.14 (1H, s)

LCMS Spectrum: m/z (ESI+)(M+H)+=374; HPLC tR=2.34 min.

### 4-(tert-Butylsulfonylmethyl)-2-chloro-6-[(3S)-3-methylmorpholin-4-yl]pyrimidine

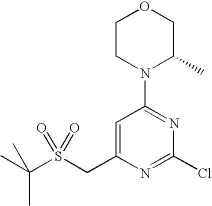

A solution of hydrogen peroxide (35% aqueous solution, 9.48 mL, 107.30 mmol) was added dropwise to a stirred solution of 4-(tert-butylsulfanylmethyl)-2-chloro-6-[(3S)-3-methylmorpholin-4-yl]pyrimidine (9.82 g, 31.1 mmol), sodium tungstate dihydrate (0.205 g, 0.62 mmol) and sulfuric acid (0.6 mL, 1M, 0.6 mmol) in dioxane (80 mL). The mixture was heated at 55° C. for 1 hour then diluted with water and cooled. A solution of sodium metabisulfite (10% w/v) was added to destroy remaining peroxide. The solution was extracted with DCM, dried (MgSO₄), filtered and concentrated in vacuo to give the desired material as a near colourless gum (9.34 g).

NMR Spectrum: ¹H NMR (399.9 MHz, CDCl₃) δ 1.33 (3H, d), 1.44 (9H, s), 3.29 (1H, dt), 3.54 (1H, dt), 3.69 (1H, dd), 3.78 (1H, d), 3.97-4.13 (2H, m), 4.21 (2H, s), 4.30 (1H, br, s), 6.71 (1H, s).

LCMS Spectrum: m/z (ESI+)(M+H)+=348; HPLC tR=1.82 min.

### 4-(tert-Butylsulfanylmethyl)-2-chloro-6-[(3S)-3-methylmorpholin-4-yl]pyrimidine

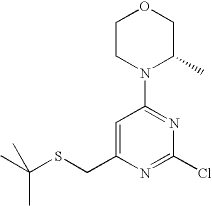

DIPEA (8.61 mL, 49.78 mmol) was added to 2-methyl-2-propanethiol (4.21 mL, 37.33 mmol), in DMF (55 mL) at RT under nitrogen. The resulting solution was stirred at RT for 20 minutes. 2-Chloro-4-(iodomethyl)-6-[(3S)-3-methylmorpholin-4-yl]pyrimidine (11.00 g, 31.11 mmol) was added to the reaction mixture in one portion. The mixture was stirred for 4 hours at RT then at 60° C. for 1.5 hours before being partitioned between ethyl acetate and water. The organic layer was washed with additional water and then dried (MgSO₄), filtered and evaporated to give the desired material as a yellow gum (10.02 g). The material was used without further purification.

NMR Spectrum: ¹H NMR (399.9 MHz, CDCl₃) δ 1.31 (3H, d), 1.34 (9H, s), 3.27 (1H, dt), 3.54 (1H, dt), 3.66-3.71 (3H, m), 3.78 (1H, d), 3.97-4.07 (2H, m), 4.31 (1H, br, s), 6.56 (1H, s)

LCMS Spectrum: m/z (ESI+)(M+H)+=316, 318; HPLC tR=2.61 min.

The preparation of 2-chloro-4-(iodomethyl)-6-[(3S)-3-methylmorpholin-4-yl]pyrimidine was described earlier.

### EXAMPLE 19

### 3-Cyclopropyl-1-[4-[4-[1-(3,5-difluorophenyl)sulfonylcyclopropyl]-6-[(3S)-3-methylmorpholin-4-yl]pyrimidin-2-yl]phenyl]urea

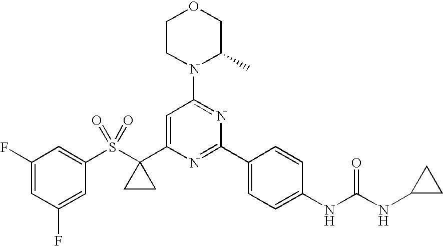

Phenyl N-[4-[4-[1-(3,5-difluorophenyl)sulfonylcyclopropyl]-6-[(3S)-3-methylmorpholin-4-yl]pyrimidin-2-yl]phenyl]carbamate (120 mg, 0.22 mmol) was added to a mixture of cyclopropylamine (59 mg, 1.03 mmol) and triethylamine (0.2 mL, 1.49 mmol) in DMF (1 mL) at RT. The reaction mixture was allowed to stand at RT for 65 hours. The crude product was purified by preparative HPLC, using decreasingly polar mixtures of water (containing 1% ammonia) and acetonitrile as eluents, to give the desired material as a colourless solid (55 mg).

NMR Spectrum: ¹H NMR (399.9 MHz, DMSO-d₆) δ 0.41-0.44 (2H, m), 0.63-0.68 (2H, m), 1.20 (3H, d), 1.62-1.69 (2H, m), 1.96-2.00 (2H, m), 2.53-2.59 (1H, m), 3.13-3.23 (1H, m), 3.42-3.50 (1H, m), 3.59-3.63 (1H, m), 3.75 (1H, d), 3.94-4.00 (1H, m), 4.18 (1H, d), 4.48 (1H, br, s), 6.42 (1H, d), 6.67 (1H, s), 7.42 (2H, d), 7.54-7.59 (2H, m), 7.70-7.76 (1H, m), 7.82 (2H, d), 8.53 (1H, s)

LCMS Spectrum: m/z (ESI+)(M+H)+=572; HPLC tR=2.72 min.

mTOR Kinase Assay (Echo): 0.00131 μM

The following compounds were made in an analogous fashion from phenyl N-[4-[4-[1-(3,5-difluorophenyl)sulfonylcyclopropyl]-6-[(3S)-3-methylmorpholin-4-yl]pyrimidin-2-yl]phenyl]carbamate and the appropriate amine.

| | | | | Reten- |
|---|---|---|---|---|
| Ex- | | | | tion |
| am- | | | LCMS | time |
| ple | Structure | NAME | MH+ | (min) |
| 19a | 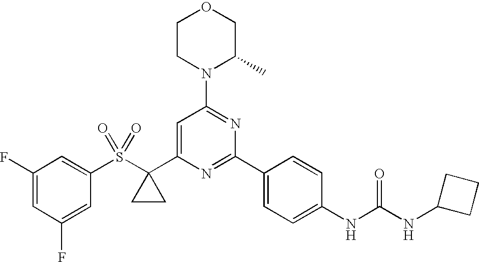 | 3-cyclobutyl-1-[4-[4-[1-(3,5-difluorophenyl)sulfonylcyclopro-pyl]-6-[(3S)-3-methylmorpholin-4-yl]pyrimidin-2-yl]phenyl]urea | 584 | 2.86 |
| 19b | 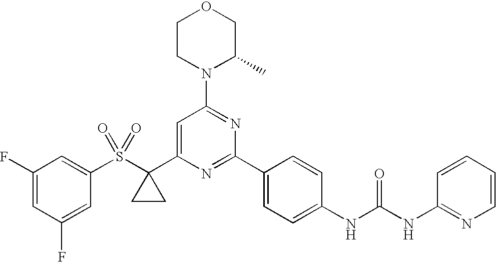 | 1-[4-[4-[1-(3,5-difluorophenyl)sulfonylcyclopro-pyl]-6-[(3S)-3-methylmorpholin-4-yl]pyrimidin-2-yl]phenyl]-3-pyridin-2-ylurea | 607 | 3.03 |
| 19c | 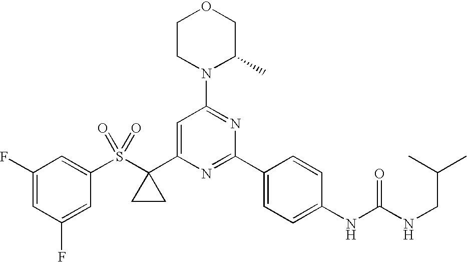 | 1-[4-[4-[1-(3,5-difluorophenyl)sulfonylcyclopro-pyl]-6-[(3S)-3-methylmorpholin-4-yl]pyrimidin-2-yl]phenyl]-3-(2-methylpropyl)urea | 586 | 2.94 |
| 19d | 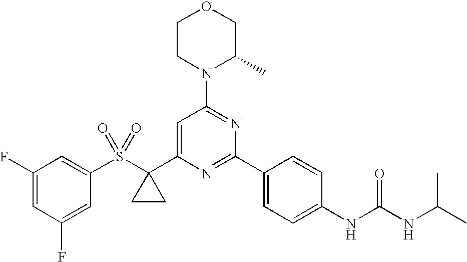 | 1-[4-[4-[1-(3,5-difluorophenyl)sulfonylcyclopro-pyl]-6-[(3S)-3-methylmorpholin-4-yl]pyrimidin-2-yl]phenyl]-3-propan-2-ylurea | 572 | 2.79 |
| 19e | 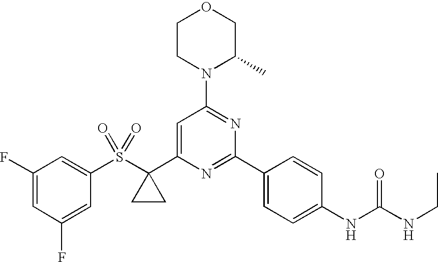 | 1-[4-[4-[1-(3,5-difluorophenyl)sulfonylcyclopro-pyl]-6-[(3S)-3-methylmorpholin-4-yl]pyrimidin-2-yl]phenyl]-3-ethylurea | 558 | 2.53 |
| 19f | 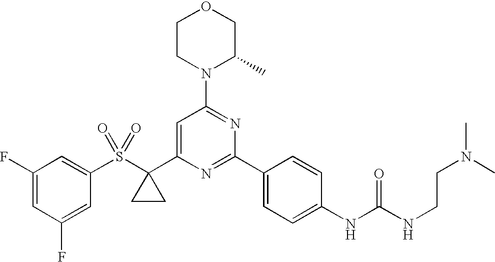 | 1-[4-[4-[1-(3,5-difluorophenyl)sulfonylcyclopro-pyl]-6-[(3S)-3-methylmorpholin-4-yl]pyrimidin-2-yl]phenyl]-3-(2-dimethylaminoethyl)urea | 601 | 2.5 |
| 19g | 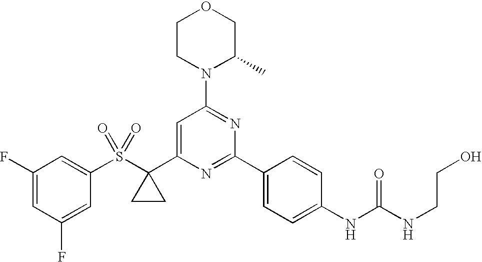 | 1-[4-[4-[1-(3,5-difluorophenyl)sulfonylcyclopro-pyl]-6-[(3S)-3-methylmorpholin-4-yl]pyrimidin-2-yl]phenyl]-3-(2-hydroxyethyl)urea | 574 | 2.17 |
| 19h | 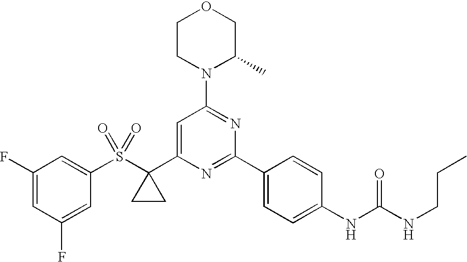 | 1-[4-[4-[1-(3,5-difluorophenyl)sulfonylcyclopro-pyl]-6-[(3S)-3-methylmorpholin-4-yl]pyrimidin-2-yl]phenyl]-3-propylurea | 572 | 2.72 |
| 19i | 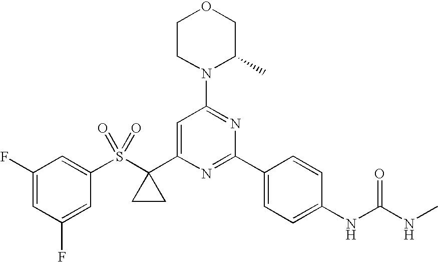 | 1-[4-[4-[1-(3,5-difluorophenyl)sulfonylcyclopro-pyl]-6-[(3S)-3-methylmorpholin-4-yl]pyrimidin-2-yl]phenyl]-3-methylurea | 544 | 2.37 |
| 19j | 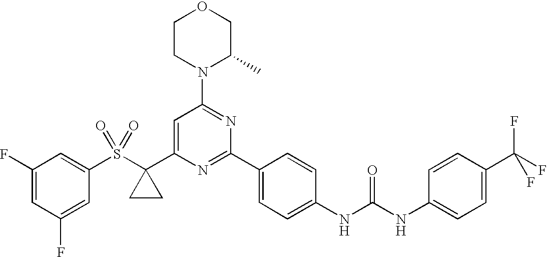 | 1-[4-[4-[1-(3,5-difluorophenyl)sulfonylcyclopro-pyl]-6-[(3S)-3-methylmorpholin-4-yl]pyrimidin-2-yl]phenyl]-3-[4-(trifluoromethyl)phenyl]urea | 674 | 3.27 |
| 19k | 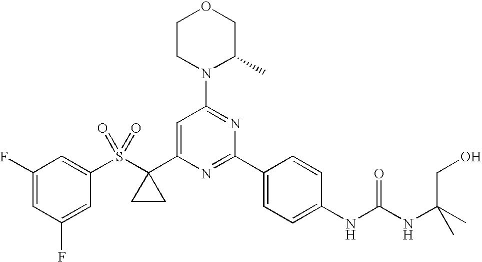 | 1-[4-[4-[1-(3,5-difluorophenyl)sulfonylcyclopro-pyl]-6-[(3S)-3-methylmorpholin-4-yl]pyrimidin-2-yl]phenyl]-3-(1-hydroxy-2-methylpropan-2-yl)urea | 602 | 2.48 |
| 19l | 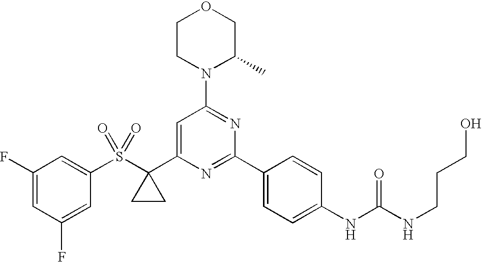 | 1-[4-[4-[1-(3,5-difluorophenyl)sulfonylcyclopro-pyl]-6-[(3S)-3-methylmorpholin-4-yl]pyrimidin-2-yl]phenyl]-3-(3-hydroxypropyl)urea | 588 | 2.23 |
| 19m | 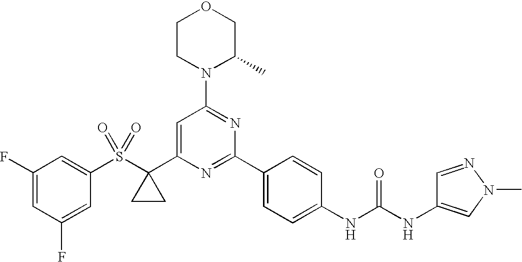 | 1-[4-[4-[1-(3,5-difluorophenyl)sulfonylcyclopro-pyl]-6-[(3S)-3-methylmorpholin-4-yl]pyrimidin-2-yl]phenyl]-3-(1-methylpyrazol-4-yl)urea | 610 | 2.39 |

### EXAMPLE 19a

¹H NMR (399.9 MHz, DMSO-d₆) δ 1.20 (3H, d), 1.58-1.70 (4H, m), 1.80-1.90 (2H, m), 1.95-2.02 (2H, m), 2.16-2.25 (2H, m), 3.10-3.20 (1H, m), 3.40-3.48 (1H, m), 3.58-3.62 (1H, m), 3.75 (1H, d), 3.92-3.98 (1H, m), 4.10-4.20 (2H, m), 4.48 (1H, br, s), 6.45 (1H, d), 6.67 (1H, s), 7.39 (2H, d), 7.55-7.57 (2H, m), 7.71-7.75 (1H, m), 7.82 (2H, d), 8.55 (1H, s).

mTOR Kinase Assay (Echo): 0.00425 μM

### EXAMPLE 19b

¹H NMR (399.9 MHz, DMSO-d₆) δ 1.21 (3H, d), 1.62-1.71 (2H, m), 1.96-2.03 (2H, m), 3.13-3.23 (1H, m), 3.42-3.51 (1H, m), 3.60-3.65 (1H, m), 3.76 (1H, d), 3.93-3.99 (1H, m), 4.19 (1H, d), 4.50 (1H, br, s), 6.71 (1H, s), 7.03-7.06 (1H, m), 7.51-7.59 (5H, m), 7.70-7.80 (2H, m), 7.92 (2H, d), 8.31 (1H, d), 9.44 (1H, s), 10.55 (1H, s).

mTOR Kinase Assay (Echo): 0.00119 μM

### EXAMPLE 19c

¹H NMR (399.9 MHz, DMSO-d₆) δ 0.89 (3H, s), 0.90 (3H, s), 1.20 (3H, d), 1.63-1.75 (3H, m), 1.94-2.00 (2H, m), 2.95 (2H, t), 3.11-3.21 (1H, m), 3.41-3.50 (1H, m), 3.57-3.63 (1H, m), 3.75 (1H, d), 3.92-3.98 (1H, m), 4.17 (1H, d), 4.48 (1H, br, s), 6.23 (1H, t), 6.67 (1H, s), 7.40 (2H, d), 7.52-7.59 (2H, m), 7.68-7.76 (1H, m), 7.83 (2H, d), 8.64 (1H, s).

mTOR Kinase Assay (Echo): 0.0214 μM

### EXAMPLE 19d

¹H NMR (399.9 MHz, DMSO-d₆) δ 1.11 (3H, s), 1.13 (3H, s), 1.20 (3H, d), 1.62-1.69 (2H, m), 1.95-2.00 (2H, m), 3.11-3.20 (1H, m), 3.42-3.50 (1H, m), 3.57-3.63 (1H, m), 3.72-3.82 (2H, m), 3.92-3.98 (1H, m), 4.17 (1H, d), 4.48 (1H, br, s), 6.05 (1H, d), 6.67 (1H, s), 7.39 (2H, d), 7.52-7.59 (2H, m), 7.69-7.76 (1H, m), 7.82 (2H, d), 8.53 (1H, s).

mTOR Kinase Assay (Echo): 0.00318 μM

### EXAMPLE 19e

¹H NMR (399.9 MHz, DMSO-d₆) δ 1.07 (3H, t), 1.20 (3H, d), 1.63-1.68 (2H, m), 1.95-2.00 (2H, m), 3.09-3.22 (3H, m), 3.37 (1H, d), 3.42-3.49 (1H, m), 3.58-3.63 (1H, m), 3.75 (1H, d), 3.93-3.98 (1H, m), 4.17 (1H, d), 4.49 (1H, br, s), 6.15 (1H, t), 6.67 (1H, s), 7.40 (2H, d), 7.54-7.57 (2H, m), 7.69-7.76 (1H, m), 7.82 (2H, d), 8.65 (1H, s).

mTOR Kinase Assay (Echo): 0.00135 μM

### EXAMPLE 19f

¹H NMR (399.9 MHz, DMSO-d₆) δ 1.20 (3H, d), 1.63-1.70 (2H, m), 1.95-2.00 (2H, m), 2.19 (6H, s), 2.34 (2H, t), 3.11-3.23 (3H, m), 3.41-3.50 (1H, m), 3.58-3.63 (1H, m), 3.75 (1H, d), 3.92-3.98 (1H, m), 4.17 (1H, d), 4.49 (1H, br, s), 6.16 (1H, t), 6.67 (1H, s), 7.40 (2H, d), 7.51-7.58 (2H, m), 7.68-7.76 (1H, m), 7.83 (2H, d), 8.88 (1H, s).

mTOR Kinase Assay (Echo): 0.0804 μM

### EXAMPLE 19g

¹H NMR (399.9 MHz, DMSO-d₆) δ 1.20 (3H, d), 1.66 (2H, s), 1.92-2.01 (2H, m), 3.11-3.20 (3H, m), 3.40-3.51 (3H, m), 3.57-3.64 (1H, m), 3.75 (1H, d), 3.92-3.99 (1H, m), 4.17 (1H, d), 4.48 (1H, br, s), 4.74 (1H, t), 6.24 (1H, t), 6.67 (1H, s), 7.40 (2H, d), 7.50-7.59 (2H, m), 7.68-7.74 (1H, t), 7.83 (2H, d), 8.79 (1H, s).

mTOR Kinase Assay (Echo): 0.000274 μM

### EXAMPLE 19h

¹H NMR (399.9 MHz, DMSO-d₆) δ 0.89 (3H, t), 1.20 (3H, d), 1.41-1.51 (2H, m), 1.66 (2H, s), 1.94-2.00 (2H, m), 3.07 (2H, q), 3.12-3.17 (1H, m), 3.42-3.49 (1H, m), 3.58-3.64 (1H, m), 3.75 (1H, d), 3.92-3.98 (1H, m), 4.17 (1H, d), 4.48 (1H, br, s), 6.20 (1H, t), 6.67 (1H, s), 7.40 (2H, d), 7.52-7.61 (2H, m), 7.68-7.76 (1H, m), 7.82 (2H, d), 8.64 (1H, s).

mTOR Kinase Assay (Echo): 0.00246 μM

### EXAMPLE 19i

¹H NMR (399.9 MHz, DMSO-d₆) δ 1.20 (3H, d), 1.61-1.70 (2H, m), 1.94-2.01 (2H, m), 2.67 (3H, d), 3.10-3.20 (1H, m), 3.40-3.49 (1H, m), 3.58-3.63 (1H, m), 3.75 (1H, d), 3.92-3.98 (1H, m), 4.17 (1H, d), 4.48 (1H, br, s), 6.02-6.08 (1H, m), 6.67 (1H, s), 7.41 (2H, d), 7.53-7.58 (2H, m), 7.68-7.75 (1H, m), 7.82 (2H, d), 8.73 (1H, s).

mTOR Kinase Assay (Echo): 0.00152 μM

### EXAMPLE 19j

¹H NMR (399.9 MHz, DMSO-d₆) δ 1.21 (3H, d), 1.67 (2H, s), 1.94-2.03 (2H, m), 3.13-3.22 (1H, m), 3.42-3.50 (1H, m), 3.59-3.64 (1H, m), 3.76 (1H, d), 3.93-3.98 (1H, m), 4.19 (1H, d), 4.49 (1H, br, s), 6.70 (1H, s), 7.50 (2H, d), 7.54-7.61 (2H, m), 7.62-7.78 (5H, m), 7.91 (2H, d), 9.04 (1H, s), 9.12 (1H, s).

mTOR Kinase Assay (Echo): 0.0199 μM

### EXAMPLE 19k

¹H NMR (399.9 MHz, DMSO-d₆) δ 1.19 (3H, d), 1.23 (6H, s), 1.62-1.68 (2H, m), 1.94-1.98 (2H, m), 3.13-3.20 (1H, m), 3.38 (2H, d), 3.42-3.50 (1H, m), 3.58-3.64 (1H, m), 3.75 (1H, d), 3.93-3.98 (1H, m), 4.17 (1H, d), 4.48 (1H, br, s), 4.94 (1H, t), 5.97 (1H, s), 7.35 (2H, d), 7.52-7.58 (2H, m), 7.69-7.77 (1H, m), 7.81 (2H, d), 8.71 (1H, s).

mTOR Kinase Assay (Echo): 0.00415 μM

### EXAMPLE 19l

¹H NMR (399.9 MHz, DMSO-d₆) δ 1.20 (3H, d), 1.55-1.70 (4H, m), 1.95-2.01 (2H, m), 3.13-3.21 (3H, m), 3.42-3.50 (3H, m), 3.58-3.65 (1H, m), 3.75 (1H, d), 3.93-3.99 (1H, m), 4.18 (1H, d), 4.43-4.52 (2H, m), 6.19 (1H, t), 6.67 (1H, s), 7.39 (2H, d), 7.52-7.61 (2H, m), 7.69-7.78 (1H, m), 7.83 (2H, d), 8.69 (1H, s).

mTOR Kinase Assay (Echo): 0.00152 μM

### EXAMPLE 19m

¹H NMR (399.9 MHz, DMSO-d₆) δ 1.20 (3H, d), 1.60-1.69 (2H, m), 1.94-2.01 (2H, m), 3.13-3.21 (1H, m), 3.42-3.50 (3H, m), 3.58-3.64 (1H, m), 3.73-3.81 (4H, m), 3.93-3.98 (1H, m), 4.18 (1H, d), 4.49 (1H, br, s), 6.67 (1H, s), 7.37 (1H, s), 7.43 (2H, d), 7.51-7.60 (2H, m), 7.68-7.78 (2H, m), 7.76 (2H, d), 8.35 (1H, s), 8.81 (1H, s).

mTOR Kinase Assay (Echo): 0.000944 μM

The preparation of phenyl N-[4-[4-[1-(3,5-difluorophenyl)sulfonylcyclopropyl]-6-[(3S)-3-methylmorpholin-4-yl]pyrimidin-2-yl]phenyl]carbamate is described below.

### Phenyl N-[4-[4-[1-(3,5-difluorophenyl)sulfonylcyclopropyl]-6-[(3S)-3-methylmorpholin-4-yl]pyrimidin-2-yl]phenyl]carbamate

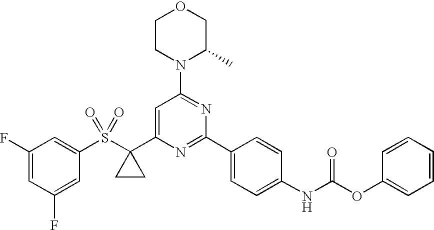

Phenyl chloroformate (0.764 mL, 6.08 mmol) was added to 4-[4-[1-(3,5-difluorophenyl)sulfonylcyclopropyl]-6-[(3S)-3-methylmorpholin-4-yl]pyrimidin-2-yl]aniline (2.69 g, 5.53 mmol) and sodium hydrogen carbonate (0.697 g, 8.29 mmol) in dioxane (40 mL) at RT. The resulting slurry was stirred at RT for 1 hour. The mixture was partitioned between ethyl acetate and water. The organic solution was dried (MgSO₄) and concentrated under reduced pressure. The residue was purified by chromatography on silica, eluting with 25%-80% ethyl acetate in isohexane, to give the desired material as a yellow dry film (3.07 g).

NMR Spectrum: ¹H NMR (399.9 MHz, CDCl₃) δ 1.32 (3H, d), 1.61-1.66 (1H, m), 1.68-1.73 (1H, m), 1.94-2.05 (2H, m), 3.30 (1H, dt), 3.61 (1H, dt), 3.75 (1H, dd), 3.84 (1H, d), 4.05 (1H, dd), 4.16 (1H, d), 4.43 (1H, br, s), 6.79 (1H, s), 6.94-7.03 (2H, m), 7.18-7.28 (3H, m), 7.30-7.35 (2H, m), 7.37-7.47 (4H, m), 8.10 (2H, d).

LCMS Spectrum: m/z (ESI+)(M+H)+=607; HPLC tR=3.12 min.

### 4-[4-[1-(3,5-Difluorophenyl)sulfonylcyclopropyl]-6-[(3S)-3-methylmorpholin-4-yl]pyrimidin-2-yl]aniline

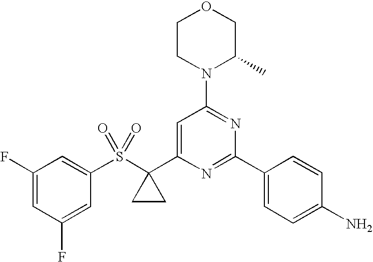

A stream of nitrogen was passed through a mixture of 2-chloro-4-[1-(3,5-difluorophenyl)sulfonylcyclopropyl]-6-[(3S)-3-methylmorpholin-4-yl]pyrimidine (2.5 g, 5.82 mmol), 4-(4,4,5,5-tetramethyl-1,3,2-dioxaborolan-2-yl)aniline (1.656 g, 7.56 mmol) and 2M aqueous sodium carbonate (10.47 mL, 20.94 mmol) in DMF (11 mL), DME (11 mL), ethanol (11 mL) and water (27.5 mL) at RT for 15 minutes. Dichlorobis(triphenylphosphine)-palladium(II) (0.204 g, 0.29 mmol) was added in one portion and the reaction mixture stirred at 80° C. under nitrogen for 1 hour. The reaction mixture was partitioned between ethyl acetate and water. The organic solution was dried (MgSO₄) and concentrated under reduced pressure. The residue was purified by chromatography on silica, eluting with 25%-60% ethyl acetate in isohexane, to give the desired material as a near colourless solid (2.76 g).

NMR Spectrum: ¹H NMR (400 MHz, CDCl₃) δ 1.31 (3H, d), 1.62-1.66 (1H, m), 1.70-1.74 (1H, m), 1.93-2.03 (2H, m), 3.28 (1H, dt), 3.59 (1H, dt), 3.72-3.74 (1H, dd), 3.79-3.89 (3H, m), 4.03 (1H, dd), 4.10-4.18 (1H, m), 4.37-4.45 (1H, m), 6.61-6.65 (2H, m), 6.73 (1H, s), 6.98 (1H, tt), 7.31-7.36 (2H, m), 7.93-7.96 (2H, m) LCMS Spectrum: m/z (ESI+)(M+H)+=487; HPLC tR=2.86 min.

### 2-Chloro-4-[1-(3,5-difluorophenyl)sulfonylcyclopropyl]-6-[(3S)-3-methylmorpholin-4-yl]pyrimidine

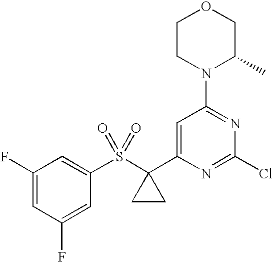

1,2-Dibromoethane (1.0 mL, 11.6 mmol) was added to 2-chloro-4-[(3,5-difluorophenyl)sulfonylmethyl]-6-[(3S)-3-methylmorpholin-4-yl]pyrimidine (3.68 g, 9.11 mmol) in toluene (55 mL) followed by tetrabutylammonium bromide (0.294 g, 0.91 mmol) and sodium hydroxide concentrate (9.00 mL, 90 mmol). The reaction mixture was stirred at RT overnight. The mixture was then heated to 80° C. and vigorous stirring continued for 3 hours. A further quantity of 1,2-dibromoethane (1 mL, 11.6 mmol) was added and heating was continued for a further 2 hours. The reaction mixture was diluted with ethyl acetate and washed with water. The organic solution was concentrated under reduced pressure. The residue was purified by chromatography on silica, eluting with 10 to 40% ethyl acetate in isohexane, to give the desired material as a colourless solid (3.0 g).

NMR Spectrum: ¹H NMR (399.9 MHz, CDCl₃) δ 1.32 (3H, d), 1.56-1.60 (1H, m), 1.63-1.68 (1H, m), 1.92-2.01 (2H, m), 3.28 (1H, dt), 3.55 (1H, dt), 3.70 (1H, dd), 3.80 (1H, d), 4.00-4.09 (2H, m), 4.28 (1H, br, s), 6.87 (1H, s), 7.07 (1H, tt), 7.24-7.29 (2H, m)

LCMS Spectrum: m/z (ESI+)(M+H)+=430, 432; HPLC tR=2.55 min.

### 2-Chloro-4-[(3,5-difluorophenyl)sulfonylmethyl]-6-[(3S)-3-methylmorpholin-4-yl]pyrimidine

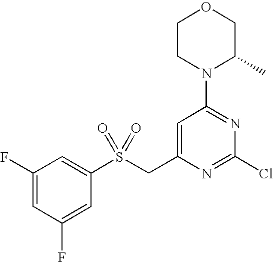

A solution of sodium tungstate dihydrate (199 mg, 0.60 mmol) in water (2 mL) was added to a stirred solution of 2-chloro-4-[(3,5-difluorophenyl)sulfanylmethyl]-6-[(3S)-3-methylmorpholin-4-yl]pyrimidine (11.23 g, 30.2 mmol) and sulfuric acid (0.302 mL, 2M, 0.60 mmol) in dioxane (40 mL). Hydrogen peroxide (3.22 mL, 104.19 mmol) was added and the mixture was stirred at RT overnight. A precipitate was collected by filtration and dried in vacuo, to give the desired material as a near colourless solid (3.61 g). The filtrate was partitioned between ethyl acetate and water. The organic solution was dried (MgSO₄) and concentrated under reduced pressure. The residue was purified by chromatography on silica, eluting with 5%-20% ethyl acetate in DCM, to give the desired material as a near colourless solid (7.66 g).

NMR Spectrum: ¹H NMR (400 MHz, CDCl₃) δ 1.34 (3H, s), 3.31 (1H, dt), 3.56 (1H, dt), 3.71 (1H, dd), 3.80 (1H, d), 3.98-4.10 (2H, m), 4.31 (2H, s), 6.55 (1H, s), 7.12 (1H, tt), 7.30-7.36 (2H, m).

LCMS Spectrum: m/z (ESI+)(M+H)+=404, 406; HPLC tR=2.32 min.

### 2-Chloro-4-[(3,5-difluorophenyl)sulfanylmethyl]-6-[(3S)-3-methylmorpholin-4-yl]pyrimidine

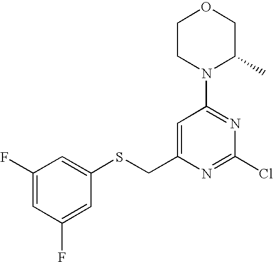

DIPEA (8.07 mL, 46.67 mmol) was added to 3,5-difluorobenzenethiol (5.00 g, 34.22 mmol), in DMF (55 mL) at RT under nitrogen. The resulting solution was stirred at RT for 20 minutes. 2-Chloro-4-(iodomethyl)-6-[(3S)-3-methylmorpholin-4-yl]pyrimidine (11.00 g, 31.11 mmol) was added to the reaction mixture in one portion. The mixture was stirred for 4 hours at RT. The reaction mixture was heated in a water bath at 60° C. for 1.5 hours before being partitioned between ethyl acetate and water. The organic solution was washed with further water then was dried (MgSO₄), filtered and evaporated to give the desired material as a gum (12.24 g).

NMR Spectrum: ¹H NMR (399.9 MHz, CDCl₃) δ 1.27 (3H, d), 3.24 (1H, dt), 3.52 (1H, dt), 3.66 (1H, dd), 3.76 (1H, d), 3.96-4.04 (4H, m), 4.21 (1H, br, s), 6.41 (1H, s), 6.59-6.66 (1H, m), 6.80-6.86 (2H, m).

LCMS Spectrum: m/z (ESI+)(M+H)+=372, 374; HPLC tR=2.66 min.

The preparation of 2-chloro-4-(iodomethyl)-6-[(3S)-3-methylmorpholin-4-yl]pyrimidine was described earlier.

### EXAMPLE 20

### 3-Cyclopropyl-1-[4-[4-[(3S)-3-methylmorpholin-4-yl]-6-(4-methylsulfonylpiperidin-4-yl)pyrimidin-2-yl]phenyl]urea

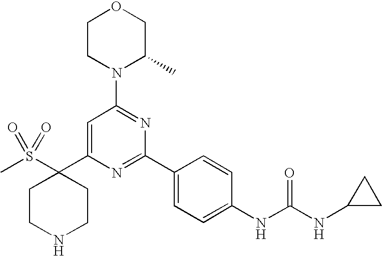

Cyclopropylamine (0.055 mL, 0.76 mmol) was added to tert-butyl 4-[6-[(3S)-3-methylmorpholin-4-yl]-2-[4-(phenoxycarbonylamino)phenyl]pyrimidin-4-yl]-4-methylsulfonylpiperidine-1-carboxylate (115 mg, 0.17 mmol) and triethylamine (0.153 mL, 1.10 mmol) in DMF (1 mL) at RT and the reaction was allowed to stand overnight at RT. The mixture was partitioned between ethyl acetate and water. The organic layer was washed twice with water then concentrated under reduced pressure. The residue was dissolved in DCM (1 mL) and treated with TFA (2 mL). The resulting solution was stirred for 30 minutes at RT before being concentrated under reduced pressure. The sample was dissolved in DMF (1.5 mL) and purified by preparative HPLC, using decreasingly polar mixtures of water (containing 1% ammonia) and acetonitrile as eluents, to give the desired material as a colourless solid (26.7 mg).

NMR Spectrum: ¹H NMR (399.9 MHz, DMSO-d₆) δ 0.38-0.44 (2H, m), 0.61-0.68 (2H, m), 1.24 (3H, d), 1.97-2.10 (2H, m), 2.30-2.45 (2H, m), 2.57 (1H, obscured by DMSO signal), 2.73-2.86 (5H, m), 2.90-3.00 (2H, m), 3.15-3.25 (1H, m), 3.47-3.55 (1H, m), 3.63-3.69 (1H, m), 3.77 (1H, d), 3.95-4.01 (1H, m), 4.29 (1H, d), 4.56 (1H, br, s), 6.45 (1H, s), 6.79 (1H, s), 7.51 (2H, d), 8.23 (2H, d), 8.57 (1H, s).

LCMS Spectrum: m/z (ESI+)(M+H)+=515; HPLC tR=1.50 min.

mTOR Kinase Assay (Echo): 0.0128 μM

The following compounds were made in an analogous fashion from tert-butyl 4-[6-[(3S)-3-methylmorpholin-4-yl]-2-[4-(phenoxycarbonylamino)phenyl]pyrimidin-4-yl]-4-methylsulfonylpiperidine-1-carboxylate and the appropriate amine.

| | | | LCMS | Retention |
|---|---|---|---|---|
| Example | Structure | NAME | MH+ | time (min) |
| 20a | 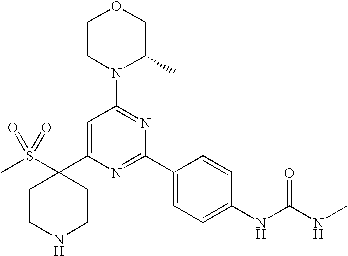 | 3-methyl-1-[4-[4-[(3S)-3-methylmorpholin-4-yl]-6-(4-methylsulfonylpiperidin-4-yl)pyrimidin-2-yl]phenyl]urea | 489.5 | 1.34 |
| 20b | 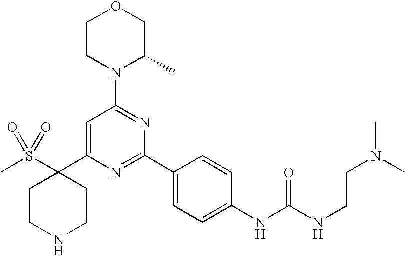 | 3-(2-dimethylaminoethyl)-1-[4-[4-[(3S)-3-methylmorpholin-4-yl]-6-(4-methylsulfonylpiperidin-4-yl)pyrimidin-2-yl]phenyl]urea | 546.5 | 1.44 |
| 20c | 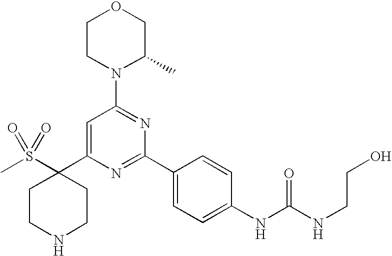 | 3-(2-hydroxyethyl)-1-[4-[4-[(3S)-3-methylmorpholin-4-yl]-6-(4-methylsulfonylpiperidin-4-yl)pyrimidin-2-yl]phenyl]urea | 519.5 | 1.24 |
| 20d | 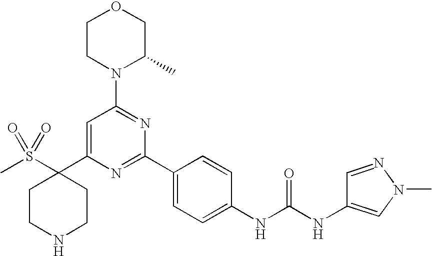 | 1-[4-[4-[(3S)-3-methylmorpholin-4-yl]-6-(4-methylsulfonylpiperidin-4-yl)pyrimidin-2-yl]phenyl]-3-(1-methylpyrazol-4-yl)urea | 555.5 | 1.41 |

### EXAMPLE 20a

¹H NMR (399.9 MHz, DMSO-d₆) δ 1.23 (3H, d), 1.97-2.09 (2H, m), 2.30-2.45 (2H, m), 2.67 (3H, d), 2.70-2.85 (5H, m), 2.90-3.02 (2H, m), 3.15-3.25 (1H, m), 3.45-3.55 (1H, m), 3.67 (1H, d), 3.77 (1H, d), 3.99 (1H, d), 4.28 (1H, d), 4.56 (1H, br, s), 6.05-6.12 (1H, m), 6.79 (1H, s), 7.51 (2H, d), 8.22 (2H, d), 8.76 (1H, s).

mTOR Kinase Assay (Echo): 0.0163 μM

### EXAMPLE 20b

¹H NMR (399.9 MHz, DMSO-d₆) δ 1.23 (3H, d), 1.98-2.09 (2H, m), 2.19 (6H, s), 2.30-2.47 (4H, m), 2.72-2.83 (5H, m), 2.92-3.02 (2H, m), 3.16-3.27 (3H, m, obscured by water signal), 3.45-3.57 (1H, m), 3.63-3.69 (1H, m), 3.77 (1H, d), 3.95-4.00 (1H, m), 4.28 (1H, d), 4.57 (1H, br, s), 6.17 (1H, t), 6.79 (1H, s), 7.49 (2H, d), 8.22 (2H, d), 8.91 (1H, s).

mTOR Kinase Assay (Echo): 0.874 μM

### EXAMPLE 20c

¹H NMR (399.9 MHz, DMSO-d₆) δ 1.23 (3H, d), 1.97-2.10 (2H, m), 2.32-2.46 (2H, m), 2.74-2.83 (5H, m), 2.92-3.02 (2H, m), 3.14-3.26 (3H, m), 3.41-3.55 (4H, m), 3.63-3.69 (1H, m), 3.77 (1H, d), 3.95-4.01 (1H, m), 4.28 (1H, d), 4.57 (1H, br, s), 4.74 (1H, t), 6.25 (1H, t), 6.79 (1H, s), 7.49 (2H, d), 8.23 (2H, d), 8.82 (1H, s).

mTOR Kinase Assay (Echo): 0.00559 μM

### EXAMPLE 20d

¹H NMR (399.9 MHz, DMSO-d₆) δ 1.24 (3H, d), 2.00-2.09 (2H, m), 2.32-2.47 (2H, m), 2.75-2.84 (5H, m), 2.92-3.02 (1H, m), 3.17-3.26 (1H, m), 3.47-3.55 (1H, m), 3.64-3.70 (1H, m), 3.75-3.82 (4H, m), 3.95-4.03 (1H, m), 4.29 (1H, d), 4.57 (1H, br, s), 6.80 (1H, s), 7.38 (1H, d), 7.55 (2H, d), 7.77 (1H, s), 8.26 (2H, d), 8.44 (1H, s), 8.89 (1H, s).

mTOR Kinase Assay (Echo): 0.0133 μM

The preparation of tert-butyl 4-[6-[(3S)-3-methylmorpholin-4-yl]-2-[4-(phenoxycarbonylamino)phenyl]pyrimidin-4-yl]-4-methylsulfonylpiperidine-1-carboxylate is described below.

### tert-Butyl 4-[6-[(3S)-3-methylmorpholin-4-yl]-2-[4-(phenoxycarbonylamino)phenyl]pyrimidin-4-yl]-4-methylsulfonylpiperidine-1-carboxylate

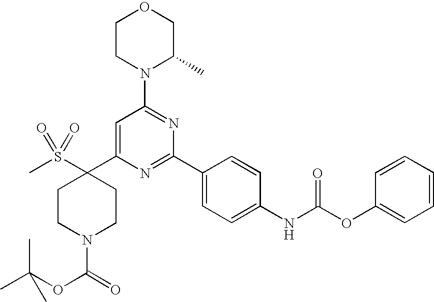

Phenyl chloroformate (0.150 mL, 1.20 mmol) was added to tert-butyl 4-[2-(4-aminophenyl)-6-[(3S)-3-methylmorpholin-4-yl]pyrimidin-4-yl]-4-methylsulfonylpiperidine-1-carboxylate (530 mg, 1.00 mmol) and sodium hydrogen carbonate (126 mg, 1.50 mmol) in dioxane (7 mL) at RT. The resulting slurry was stirred at RT for 3 hours. The mixture was partitioned between ethyl acetate and water. The organic solution was dried (MgSO₄) and concentrated under reduced pressure. The residue was purified by chromatography on silica, eluting with 10%-60% ethyl acetate in DCM, to give a colourless gum which was subsequently triturated with diethyl ether to give the desired material as a colourless solid (576 mg).

NMR Spectrum: ¹H NMR (399.9 MHz, CDCl₃) δ 1.36 (3H, d), 1.44 (9H, s), 2.29-2.41 (2H, m), 2.72 (3H, s), 2.73-2.85 (4H, m), 3.33 (1H, dt), 3.61 (1H, dt), 3.76 (1H, dd), 3.84 (1H, d), 4.06 (1H, dd), 4.10-4.32 (3H, m), 4.46 (1H, br, s), 6.65 (1H, s), 7.12 (1H, br, s), 7.17-7.28 (3H, m), 7.36-7.44 (2H, m), 7.54 (2H, d), 8.35 (2H, d).

LCMS Spectrum: m/z (ESI+)(M+H)+=652; HPLC tR=3.11 min.

### tert-Butyl 4-[2-(4-aminophenyl)-6-[(3S)-3-methylmorpholin-4-yl]pyrimidin-4-yl]-4-methylsulfonylpiperidine-1-carboxylate

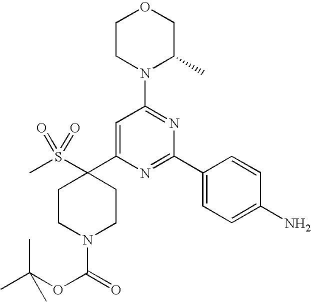

A stream of nitrogen was passed through tert-butyl 4-[2-chloro-6-[(3S)-3-methylmorpholin-4-yl]pyrimidin-4-yl]-4-methylsulfonylpiperidine-1-carboxylate (500 mg, 1.05 mmol), 4-(4,4,5,5-tetramethyl-1,3,2-dioxaborolan-2-yl)aniline (300 mg, 1.37 mmol) and 2M aqueous sodium carbonate (1.895 mL, 3.79 mmol) in DMF (2 mL), DME (2 mL), ethanol (2 mL) and water (5 mL) at RT for 15 minutes. The reaction mixture was treated with dichlorobis(triphenylphosphine)-palladium(II) (36.9 mg, 0.05 mmol) and the mixture was stirred at 80° C. for 30 minutes. The mixture was partitioned between ethyl acetate and water. The organic solution was dried (MgSO₄), filtered and concentrated under reduced pressure. The residue was purified by chromatography on silica, eluting with 10%-50% ethyl acetate in DCM. The isolated material was triturated with diethyl ether to give the desired material as a pale orange solid (544 mg).

NMR Spectrum: ¹H NMR (399.9 MHz, CDCl₃) δ 1.34 (3H, d), 1.44 (9H, s), 2.30-2.36 (2H, m), 2.71 (3H, s), 2.69-2.85 (4H, m), 3.30 (1H, dt), 3.60 (1H, dt), 3.74 (1H, dd), 3.83 (1H, d), 3.93 (2H, s), 4.04 (1H, dd), 4.09-4.29 (3H, m), 4.45 (1H, br, s), 6.58 (1H, s), 6.71 (2H, d), 8.18 (2H, d)

LCMS Spectrum: m/z (ESI+)(M+H)+=532; HPLC tR=2.52 min.

### tert-Butyl 4-[2-chloro-6-[(3S)-3-methylmorpholin-4-yl]pyrimidin-4-yl]-4-methylsulfonylpiperidine-1-carboxylate

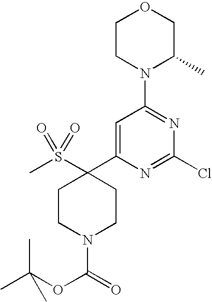

1-Chloroethyl chloroformate (0.315 mL, 292 mmol) was added to a solution of 4-(1-benzyl-4-methylsulfonylpiperidin-4-yl)-2-chloro-6-[(3S)-3-methylmorpholin-4-yl]pyrimidine (679 mg, 1.46 mmol) in DCM (10 mL). The solution was heated at reflux for 3 hours. The mixture was cooled, diluted with methanol (10 mL) and allowed to stand overnight. The mixture was treated with di-tert-butyl dicarbonate (0.738 mL, 3.21 mmol) and DIPEA (0.505 mL, 2.92 mmol) and this solution was stirred at RT for 1.5 hours. The solution was partitioned between DCM and water and the organic phase concentrated under reduced pressure. The residue was purified by chromatography on silica, eluting with 10%-30% ethyl acetate in DCM, to give the desired material as a colourless dry film (519 mg).

NMR Spectrum: ¹H NMR (399.9 MHz, CDCl₃) δ 1.33 (3H, d), 1.45 (9H, s), 2.23-2.34 (2H, m), 2.59-2.78 (7H, m), 3.30 (1H, dt), 3.55 (1H, dt), 3.70 (1H, dd), 3.80 (1H, d), 3.98-4.40 (5H, m), 6.61 (1H, s)

LCMS Spectrum: m/z (ESI+)(M+H)+=475, 477; HPLC tR=2.53 min.

### 4-(1-Benzyl-4-methylsulfonylpiperidin-4-yl)-2-chloro-6-[(3S)-3-methylmorpholin-4-yl]pyrimidine

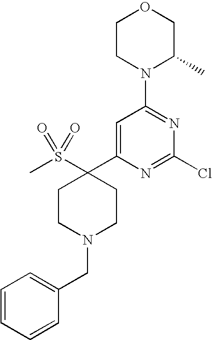

A solution of 2-chloro-4-[(3S)-3-methylmorpholin-4-yl]-6-(methylsulfonylmethyl)pyrimidine (750 mg, 2.45 mmol) in NMP (8 mL) was treated with sodium hydride (324 mg, 8.10 mmol). The mixture was stirred at RT for 10 minutes before being treated with tetrabutylammonium bromide (979 mg, 3.04 mmol) and N-benzyl-2-chloro-N-(2-chloroethyl)ethanamine hydrochloride (692 mg, 2.58 mmol). The reaction mixture was stirred for 5 minutes, warmed to 50° C. for 1 hour then warmed to 80° C. for 2.5 hours. The mixture was allowed to cool and stirred for 65 hours at RT. The reaction mixture was quenched by the addition of saturated aqueous ammonium chloride solution and then extracted with ethyl acetate. The organic solution was dried (MgSO₄), filtered, and concentrated under reduced pressure. The residue was purified by chromatography on silica, eluting with 10%-50% ethyl acetate in DCM, to give the desired material as a colourless solid (710 mg).

NMR Spectrum: ¹H NMR (399.9 MHz, CDCl₃) δ 1.33 (3H, d), 1.89-1.99 (2H, m), 2.40-2.50 (2H, m), 2.57-2.64 (2H, m), 2.75 (3H, s), 2.87-2.95 (2H, m), 3.29 (1H, dt), 3.41 (2H, s), 3.55 (1H, dt), 3.69 (1H, dd), 3.79 (1H, d), 3.95-4.08 (2H, m), 4.29 (1H, br, s), 6.59 (1H, s), 7.21-7.32 (5H, m)

LCMS Spectrum: m/z (ESI+)(M+H)+=465, 467; HPLC tR=2.59 min.

The preparation of 2-chloro-4-[(3S)-3-methylmorpholin-4-yl]-6-(methylsulfonylmethyl)pyrimidine was described earlier.

### EXAMPLE 21

### 3-Cyclopropyl-1-[4-[4-(4-cyclopropylsulfonylpiperidin-4-yl)-6-[(3S)-3-methylmorpholin-4-yl]pyrimidin-2-yl]phenyl]urea

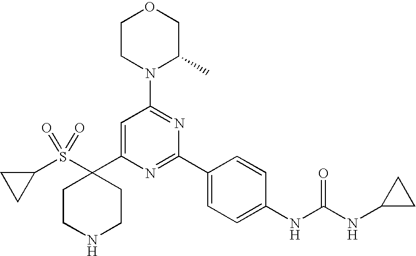

Cyclopropylamine (0.055 mL, 0.76 mmol) was added to tert-butyl 4-cyclopropylsulfonyl-4-[6-[(3S)-3-methylmorpholin-4-yl]-2-[4-(phenoxycarbonylamino)phenyl]pyrimidin-4-yl]piperidine-1-carboxylate (120 mg, 0.18 mmol) and triethylamine (0.16 mL, 1.15 mmol) in DMF (1 mL) at RT and the reaction was allowed to stand for overnight at RT. The mixture was partitioned between ethyl acetate and water. The organic layer was washed twice with water then concentrated under reduced pressure. The residue was dissolved in DCM (1 mL) and treated with TFA (2 mL). The resulting solution was stirred for 30 minutes at RT before being concentrated under reduced pressure. The sample was dissolved in DMF (1.5 mL). The crude product was purified by preparative HPLC, using decreasingly polar mixtures of water (containing 1% conc. ammonia) and acetonitrile as eluents, to give the desired material as a colourless solid (53 mg).

NMR Spectrum: ¹H NMR (399.9 MHz, DMSO-d₆) δ 0.38-0.44 (2H, m), 0.63-0.68 (2H, m), 0.70-0.74 (2H, m), 0.83-0.87 (2H, m), 1.23 (3H, d), 1.98-2.12 (2H, m), 2.32-2.60 (4H, m), 2.82-2.98 (4H, m), 3.15-3.24 (1H, m), 3.47-3.56 (1H, m), 3.64-3.69 (1H, m), 3.77 (1H, d), 3.95-4.01 (1H, m), 4.26 (1H, d), 4.50-4.60 (1H, m), 6.44 (1H, s), 6.81 (1H, s), 7.51 (2H, d), 8.23 (2H, d), 8.54 (1H, s).

LCMS Spectrum: m/z (ESI+)(M+H)+=541; HPLC tR=1.85 min.

mTOR Kinase Assay (Echo): 0.0253 μM

The following compounds were made in an analogous fashion from tert-butyl 4-cyclopropylsulfonyl-4-[6-[(3S)-3-methylmorpholin-4-yl]-2-[4-(phenoxycarbonylamino)phenyl]pyrimidin-4-yl]piperidine-1-carboxylate and the appropriate amine.

| | | | LCMS | Retention |
|---|---|---|---|---|
| Example | Structure | NAME | MH+ | time (min) |
| 21a | 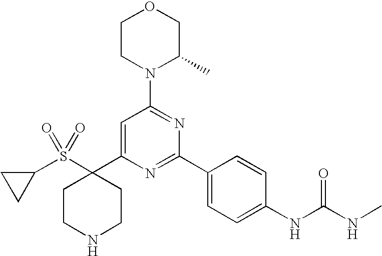 | 1-[4-[4-(4-cyclopropylsulfonylpiperidin-4-yl)-6-[(3S)-3-methylmorpholin-4-yl]pyrimidin-2-yl]phenyl]-3-methylurea | 515.6 | 1.71 |
| 21b | 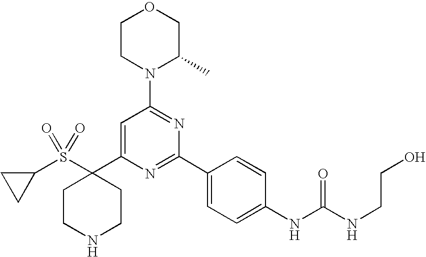 | 1-[4-[4-(4-cyclopropylsulfonylpiperidin-4-yl)-6-[(3S)-3-methylmorpholin-4-yl]pyrimidin-2-yl]phenyl]-3-(2-hydroxyethyl)urea | 545.7 | 1.56 |
| 21c | 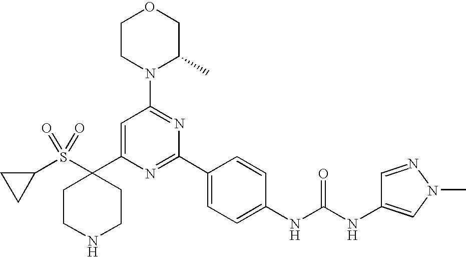 | 1-[4-[4-(4-cyclopropylsulfonylpiperidin-4-yl)-6-[(3S)-3-methylmorpholin-4-yl]pyrimidin-2-yl]phenyl]-3-(1-methylpyrazol-4-yl)urea | 581.7 | 1.83 |

### EXAMPLE 21a

¹H NMR (399.9 MHz, DMSO-d₆) δ 0.68-0.74 (2H, m), 0.83-0.87 (2H, m), 1.23 (3H, d), 1.98-2.10 (2H, m), 2.32-2.49 (3H, m), 2.67 (3H, d), 2.82-2.97 (4H, m), 3.15-3.24 (1H, m), 3.47-3.57 (1H, m), 3.64-3.69 (1H, m), 3.77 (1H, d), 3.95-4.01 (1H, m), 4.26 (1H, d), 4.50-4.59 (1H, m), 6.07 (1H, q), 6.81 (1H, s), 7.50 (2H, d), 8.22 (2H, d), 8.74 (1H, s).

mTOR Kinase Assay (Echo): 0.0378 μM

### EXAMPLE 21b

¹H NMR (399.9 MHz, DMSO-d₆) δ 0.69-0.76 (2H, m), 0.82-0.89 (2H, m), 1.23 (3H, d), 1.95-2.10 (2H, m), 2.31-2.49 (3H, m), 2.83-2.97 (4H, m), 3.15-3.26 (3H, m), 3.43-3.58 (3H, m), 3.64-3.69 (1H, m), 3.77 (1H, d), 3.95-4.01 (1H, m), 4.26 (1H, d), 4.50-4.59 (1H, m), 4.74 (1H, t), 6.26 (1H, t), 6.81 (1H, s), 7.49 (2H, d), 8.23 (2H, d), 8.81 (1H, s).

mTOR Kinase Assay (Echo): 0.0133 μM

### EXAMPLE 21c

¹H NMR (399.9 MHz, DMSO-d₆) δ 0.73 (2H, br, s), 0.87 (2H, d), 1.23 (3H, d), 1.99-2.11 (2H, m), 2.33-2.48 (3H, m), 2.83-2.98 (4H, m), 3.15-3.20 (1H, m), 3.45-3.56 (1H, m), 3.68 (1H, d), 3.74-3.83 (4H, m), 3.99 (1H, d), 4.27 (1H, d), 4.55 (1H, br, s), 6.82 (1H, s), 7.38 (1H, s), 7.55 (2H, d), 7.76 (1H, s), 8.26 (2H, d), 8.42 (1H, s), 8.86 (1H, s).

mTOR Kinase Assay (Echo): 0.0234 μM

The preparation of tert-butyl 4-cyclopropylsulfonyl-4-[6-[(3S)-3-methylmorpholin-4-yl]-2-[4-(phenoxycarbonylamino)phenyl]pyrimidin-4-yl]piperidine-1-carboxylate is described below.

### tert-Butyl 4-cyclopropylsulfonyl-4-[6-[(3S)-3-methylmorpholin-4-yl]-2-[4-(phenoxycarbonylamino)phenyl]pyrimidin-4-yl]piperidine-1-carboxylate

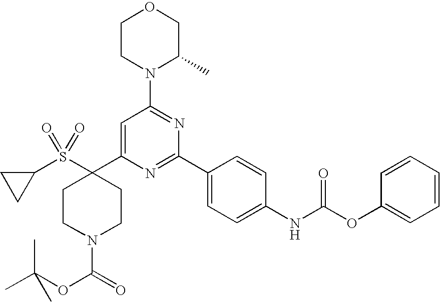

Phenyl chloroformate (0.235 mL, 1.87 mmol) was added to tert-butyl 4-[2-(4-aminophenyl)-6-[(3S)-3-methylmorpholin-4-yl]pyrimidin-4-yl]-4-cyclopropylsulfonylpiperidine-1-carboxylate (950 mg, 1.70 mmol) and sodium hydrogen carbonate (215 mg, 2.56 mmol) in dioxane (15 mL) at RT. The resulting slurry was stirred at RT for 1 hour. The mixture was partitioned between ethyl acetate and water. The organic solution was dried (MgSO₄) and concentrated under reduced pressure. The residue was purified by chromatography on silica, eluting with 25%-80% ethyl acetate in isohexane. The isolated material was triturated with diethyl ether to give the desired material as a near colourless dry film (1.06 g).

NMR Spectrum: ¹H NMR (399.9 MHz, CDCl₃) δ 0.80-0.85 (2H, m), 0.94-1.06 (2H, m), 1.35 (3H, d), 1.44 (9H, s), 2.14-2.22 (1H, m), 2.25-2.39 (2H, m), 2.69-2.95 (4H, m), 3.33 (1H, dt), 3.62 (1H, dt), 3.76 (1H, dd), 3.84 (1H, d), 4.03-4.31 (4H, m), 4.39-4.49 (1H, m), 6.68 (1H, s), 7.11 (1H, br, s), 7.19-7.28 (3H, m), 7.41 (2H, t), 7.54 (2H, d), 8.37 (2H, d).

LCMS Spectrum: m/z (ESI+)(M+H)+=678; HPLC tR=3.18 min.

### tert-Butyl 4-[2-(4-aminophenyl)-6-[(3S)-3-methylmorpholin-4-yl]pyrimidin-4-yl]-4-cyclopropylsulfonylpiperidine-1-carboxylate

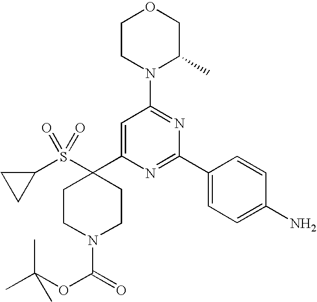

A stream of nitrogen was passed through tert-butyl 4-[2-chloro-6-[(3S)-3-methylmorpholin-4-yl]pyrimidin-4-yl]-4-cyclopropylsulfonylpiperidine-1-carboxylate (0.94 g, 1.88 mmol), 4-(4,4,5,5-tetramethyl-1,3,2-dioxaborolan-2-yl)aniline (0.534 g, 2.44 mmol) and 2M aqueous sodium carbonate (3.38 mL, 6.75 mmol) in DMF (3.75 mL), DME (5 mL), ethanol (5 mL) and water (12.5 mL) at RT for 15 minutes. The reaction mixture was treated with dichlorobis(triphenylphosphine)-palladium(II) (0.066 g, 0.09 mmol) and the mixture was stirred at 80° C. for 1 hour. The mixture was partitioned between ethyl acetate and water. The organic solution was dried (MgSO₄) and concentrated under reduced pressure. The residue was purified by chromatography on silica, eluting with 10%-50% ethyl acetate in DCM. The isolated material was triturated with diethyl ether to give the desired material as a pale brown solid (0.990 g).

NMR Spectrum: ¹H NMR (399.9 MHz, CDCl₃) δ 0.77-0.85 (2H, m), 0.95-1.04 (2H, m), 1.32 (3H, d), 1.42 (9H, s), 2.14-2.21 (1H, m), 2.24-2.37 (2H, m), 2.69-2.93 (4H, m), 3.29 (1H, dt), 3.60 (1H, dt), 3.75 (1H, dd), 3.81 (1H, d), 3.90 (2H, s), 4.04 (1H, dd), 4.07-4.30 (3H, m), 4.39-4.48 (1H, m), 6.61 (1H, s), 6.61 (2H, d), 8.20 (2H, d).

LCMS Spectrum: m/z (ESI+)(M+H)+=558; HPLC tR=2.64 min.

### tert-Butyl 4-[2-chloro-6-[(3S)-3-methylmorpholin-4-yl]pyrimidin-4-yl]-4-cyclopropylsulfonylpiperidine-1-carboxylate

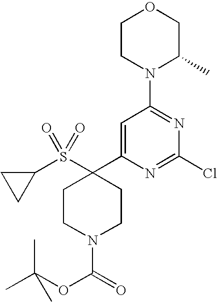

1-Chloroethyl chloroformate (0.971 mL, 9.00 mmol) was added to a solution of 4-(1-benzyl-4-cyclopropylsulfonylpiperidin-4-yl)-2-chloro-6-[(3S)-3-methylmorpholin-4-yl]pyrimidine (2.21 g, 4.50 mmol) in DCM (15 mL). The solution was heated at reflux for 1.5 hours. The mixture was diluted with methanol (15 mL) and heating was continued for 2 hours. The mixture was treated with di-tert-butyl dicarbonate (2.16 g, 9.90 mmol) and DIPEA (1.6 mL, 9.0 mmol) and this solution was stirred at RT for 1 hour. The solution was partitioned between DCM and water. The organic phase was concentrated under reduced pressure and the residue was purified by chromatography on silica, eluting with 10%-30% ethyl acetate in DCM, to give the desired material as a colourless solid (1.9 g).

NMR Spectrum: ¹H NMR (399.9 MHz, CDCl₃) δ 0.93-1.00 (4H, m), 1.32 (3H, d), 1.44 (9H, s), 2.19-2.30 (3H, m), 2.62-2.80 (4H, m), 3.29 (1H, dt), 3.55 (1H, dt), 3.69 (1H, dd), 3.79 (1H, d), 3.95-4.37 (5H, m), 6.65 (1H, s).

LCMS Spectrum: m/z (ESI+)(M+H)+=501, 503; HPLC tR=2.70 min.

### 4-(1-Benzyl-4-cyclopropylsulfonylpiperidin-4-yl)-2-chloro-6-[(3S)-3-methylmorpholin-4-yl]pyrimidine

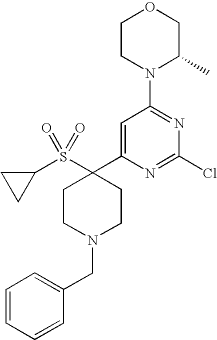

A solution of 2-chloro-4-(cyclopropylsulfonylmethyl)-6-[(3S)-3-methylmorpholin-4-yl]pyrimidine (2 g, 6.03 mmol) in NMP (18 mL) was treated with sodium hydride (0.796 g, 19.89 mmol). The mixture was stirred at RT for 10 minutes before being treated with tetrabutylammonium bromide (2.91 g, 9.04 mmol) and N-benzyl-2-chloro-N-(2-chloroethyl)ethanamine hydrochloride (1.781 g, 6.63 mmol). The reaction mixture was stirred for 5 minutes, warmed to 50° C. for 1 hour then warmed to 80° C. for 1.5 hours. The mixture was then allowed to cool to RT. The reaction mixture was quenched by the addition of saturated aqueous ammonium chloride solution and then extracted with ethyl acetate. The organic solution was washed three times with water then dried (MgSO₄), filtered, and concentrated under reduced pressure. The residue was purified by chromatography on silica, eluting with 10%-70% ethyl acetate in DCM, to give the desired material as a colourless foam (2.23 g).

NMR Spectrum: ¹H NMR (399.9 MHz, CDCl₃) δ 0.92-0.96 (2H, m), 0.97-1.02 (2H, m), 1.32 (3H, d), 1.92-2.00 (2H, m), 2.24-2.31 (1H, m), 2.40-2.49 (2H, m), 2.68-2.74 (2H, m), 2.88-2.92 (2H, m), 3.29 (1H, dt), 3.40 (2H, s), 3.55 (1H, dt), 3.70 (1H, dd), 3.79 (1H, d), 3.98-4.09 (2H, m), 4.28 (1H, br, s), 6.63 (1H, s), 7.21-7.33 (5H, m)

LCMS Spectrum: m/z (ESI+)(M+H)+=491, 493; HPLC tR=2.71 min.

The preparation of 2-chloro-4-(cyclopropylsulfonylmethyl)-6-[(3S)-3-methylmorpholin-4-yl]pyrimidine was described earlier.

### EXAMPLE 22

### 1-[4-[4-[1-Benzyl-4-(3,5-difluorophenyl)sulfonylpiperidin-4-yl]-6-[(3S)-3-methylmorpholin-4-yl]pyrimidin-2-yl]phenyl]-3-cyclopropylurea

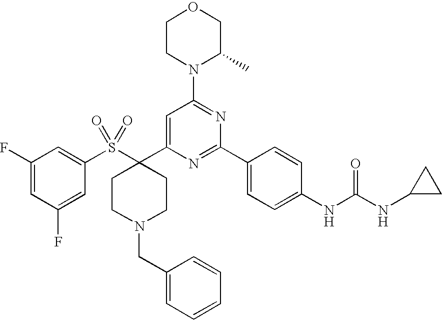

A stream of nitrogen was passed through [4-(3-cyclopropylureido)phenylboronic acid, pinacol ester (121 mg, 0.40 mmol), 4-[1-benzyl-4-(3,5-difluorophenyl)sulfonylpiperidin-4-yl]-2-chloro-6-[(3S)-3-methylmorpholin-4-yl]pyrimidine (225 mg, 0.40 mmol) and 2M aqueous sodium carbonate (0.719 mL, 1.44 mmol) in DMF (0.8 mL), DME (5 mL), ethanol (5 mL) and water (12.5 mL) at 25° C. for 15 minutes. The reaction mixture was treated with dichlorobis(triphenylphosphine)-palladium(II) (14.02 mg, 0.02 mmol) and the mixture was stirred at 80° C. for 2 hours. The mixture was partitioned between DCM and water. The organic solution was dried (MgSO₄) and concentrated under reduced pressure. The residue was purified by ion exchange chromatography using an SCX column, eluting with 2M ammonia in methanol. The isolated material was further purified by chromatography on silica, eluting with 10%-60% ethyl acetate in DCM, to give the desired material as a colourless dry film (125 mg).

NMR Spectrum: ¹H NMR (399.9 MHz, CDCl₃) δ 0.67-0.71 (2H, m), 0.85-0.90 (2H, m), 1.36 (3H, d), 1.77-1.98 (2H, m), 2.47-2.67 (3H, m), 2.74 (2H, t), 2.85-2.96 (2H, m), 3.30-3.38 (3H, m), 3.64 (1H, dt), 3.76-3.87 (2H, m), 4.08 (1H, dd), 4.16 (1H, d), 4.40-4.51 (1H, m), 4.92 (1H, s), 6.63 (1H, s), 6.80-6.86 (1H, m), 6.95-7.03 (3H, m), 7.20-7.31 (5H, m), 7.39 (2H, d), 7.93 (2H, d).

LCMS Spectrum: m/z (ESI+)(M+H)+=703.6; HPLC tR=2.97 min.

mTOR Kinase Assay (Echo): 0.503 μM

The preparation of 4-[1-benzyl-4-(3,5-difluorophenyl)sulfonylpiperidin-4-yl]-2-chloro-6-[(3S)-3-methylmorpholin-4-yl]pyrimidine is described below.

### 4-[1-Benzyl-4-(3,5-difluorophenyl)sulfonylpiperidin-4-yl]-2-chloro-6-[(3S)-3-methylmorpholin-4-yl]pyrimidine

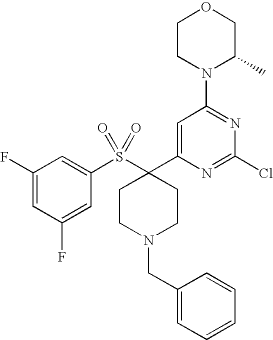

A mixture of 2-chloro-4-[(3,5-difluorophenyl)sulfonylmethyl]-6-[(3S)-3-methylmorpholin-4-yl]pyrimidine (874 mg, 2.16 mmol) in NMP (10 mL) was treated with sodium hydride (299 mg, 7.47 mmol) and stirred for 5 minutes at RT. Tetrabutylammonium bromide (698 mg, 2.16 mmol) and N-benzyl-2-chloro-N-(2-chloroethyl)ethanamine hydrochloride (669 mg, 2.49 mmol) were added and the mixture heated at 80° C. for 2 hours. The reaction mixture was quenched by the addition of saturated aqueous ammonium chloride solution and then extracted with ethyl acetate. The organic solution was dried (MgSO₄), filtered, and concentrated under reduced pressure. The residue was purified by chromatography on silica, eluting with 10%-50% ethyl acetate in isohexane, to give the desired material as a colourless solid (582 mg).

NMR Spectrum: ¹H NMR (399.9 MHz, CDCl₃) δ 1.35 (3H, d), 1.85-1.93 (2H, m), 2.41-2.49 (2H, m), 2.55 (2H, d), 2.88-2.93 (2H, m), 3.32 (1H, dt), 3.38 (2H, s), 3.59 (1H, dt), 3.74 (1H, dd), 3.81 (1H, d), 3.98-4.08 (2H, m), 4.31 (1H, br, s), 6.64 (1H, s), 6.97-7.11 (3H, m), 7.22-7.33 (5H, m).

LCMS Spectrum: m/z (ESI+)(M+H)+=563, 565; HPLC tR=3.18 min.

The preparation of 2-chloro-4-[(3,5-difluorophenyl)sulfonylmethyl]-6-[(3S)-3-methylmorpholin-4-yl]pyrimidine was described earlier.

### EXAMPLE 23

### 3-Cyclopropyl-1-[4-[4-[(3S)-3-methylmorpholin-4-yl]-6-(4-methylsulfonyloxan-4-yl)pyrimidin-2-yl]phenyl]urea

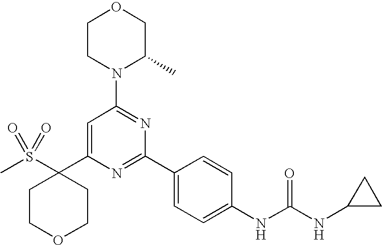

Cyclopropylamine (0.100 mL, 1.45 mmol) was added to phenyl N-[4-[4-[(3S)-3-methylmorpholin-4-yl]-6-(4-methylsulfonyloxan-4-yl)pyrimidin-2-yl]phenyl]carbamate (100 mg, 0.18 mmol) in DMF (2 mL). The resulting solution was stirred at 60° C. for 4 hours. The mixture was evaporated to dryness and the residue was purified by preparative HPLC, using decreasingly polar mixtures of water (containing 1% ammonia) and acetonitrile as eluents, to give the desired material as a white solid (67 mg).

NMR Spectrum: ¹H NMR (399.902 MHz, CDCl₃) δ 0.71 (2H, ddd), 0.89 (2H, ddd), 1.35 (3H, d), 2.54 (3H, ddd), 2.64 (1H, m), 2.71 (3H, s), 2.72 (2H, br.d), 3.34 (1H, ddd), 3.42 (1H, ddd), 3.62 (1H, ddd), 3.77 (1H, dd), 3.84 (1H, d), 4.05 (3H, m), 4.20 (1H, d), 4.46 (1H, br.d), 4.89 (1H, s), 6.64 (1H, s), 7.00 (1H, s), 7.51 (2H, d), 8.31 (2H, d).

LCMS Spectrum: m/z (ESI+)(M+H)+=516; HPLC tR=1.92 min.

mTOR Kinase Assay (Echo): 0.00492 μM

The following compounds were made in an analogous fashion from either phenyl N-[4-[4-[(3S)-3-methylmorpholin-4-yl]-6-(4-methylsulfonyloxan-4-yl)pyrimidin-2-yl]phenyl]carbamate, phenyl N-[4-[4-(4-cyclopropylsulfonyloxan-4-yl)-6-[(3S)-3-methylmorpholin-4-yl]pyrimidin-2-yl]phenyl]carbamate or phenyl N-[4-[4-[4-(4-chlorophenyl)sulfonyloxan-4-yl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 23a | 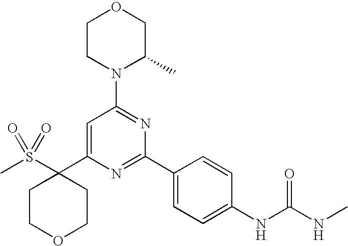 | 3-methyl-1-[4-[4-[(3S)-3-methylmorpholin-4-yl]-6-(4-methylsulfonyloxan-4-yl)pyrimidin-2-yl]phenyl]urea | 490 | 1.77 |
| 23b | 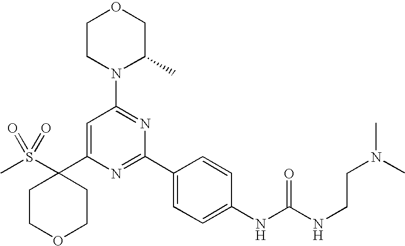 | 3-(2-dimethylaminoethyl)-1-[4-[4-[(3S)-3-methylmorpholin-4-yl]-6-(4-methylsulfonyloxan-4-yl)pyrimidin-2-yl]phenyl]urea | 547 | 1.84 |
| 23c | 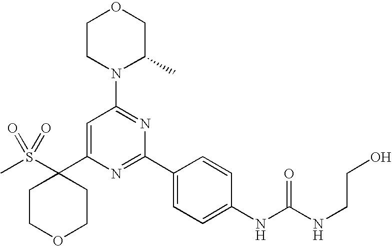 | 3-(2-hydroxyethyl)-1-[4-[4-[(3S)-3-methylmorpholin-4-yl]-6-(4-methylsulfonyloxan-4-yl)pyrimidin-2-yl]phenyl]urea | 520 | 1.61 |
| 23d | 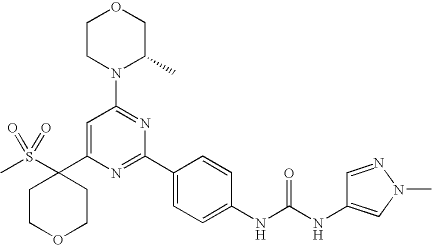 | 1-[4-[4-[(3S)-3-methylmorpholin-4-yl]-6-(4-methylsulfonyloxan-4-yl)pyrimidin-2-yl]phenyl]-3-(1-methylpyrazol-4-yl)urea | 554(M − H)− | 1.83 |
| 23e | 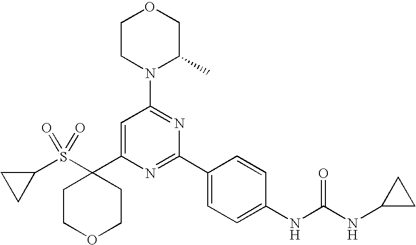 | 3-cyclopropyl-1-[4-[4-(4-cyclopropylsulfonyloxan-4-yl)-6-[(3S)-3-methylmorpholin-4-yl]pyrimidin-2-yl]phenyl]urea | 542 | 2.07 |
| 23f | 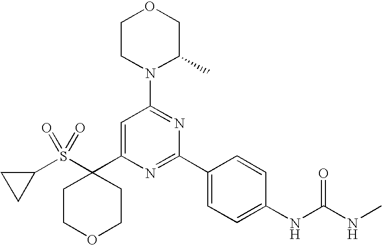 | 1-[4-[4-(4-cyclopropylsulfonyloxan-4-yl)-6-[(3S)-3-methylmorpholin-4-yl]pyrimidin-2-yl]phenyl]-3-methylurea | 516 | 1.89 |
| 23g | 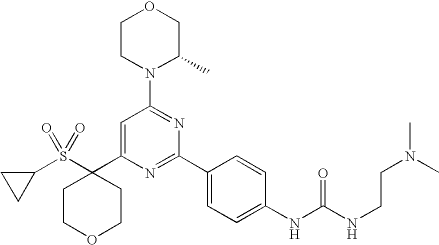 | 1-[4-[4-(4-cyclopropylsulfonyloxan-4-yl)-6-[(3S)-3-methylmorpholin-4-yl]pyrimidin-2-yl]phenyl]-3-(2-dimethylaminoethyl)urea | 573 | 1.96 |
| 23h | 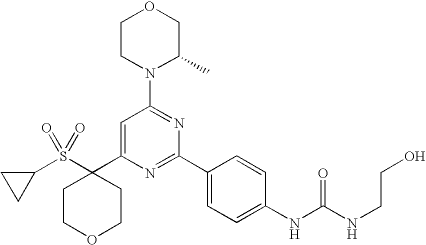 | 1-[4-[4-(4-cyclopropylsulfonyloxan-4-yl)-6-[(3S)-3-methylmorpholin-4-yl]pyrimidin-2-yl]phenyl]-3-(2-hydroxyethyl)urea | 546 | 1.72 |
| 23i | 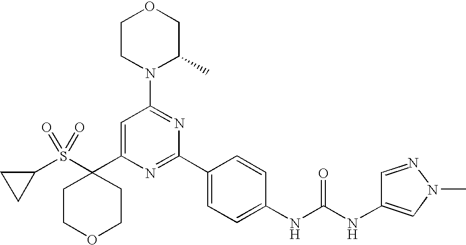 | 1-[4-[4-(4-cyclopropylsulfonyloxan-4-yl)-6-[(3S)-3-methylmorpholin-4-yl]pyrimidin-2-yl]phenyl]-3-(1-methylpyrazol-4-yl)urea | 580(M − H)− | 1.93 |
| 23j | 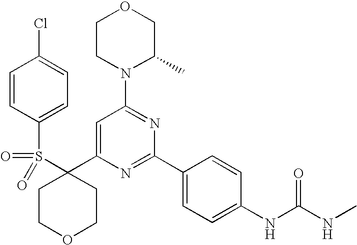 | 1-[4-[4-[4-(4-chlorophenyl)sulfonyloxan-4-yl]-6-[(3S)-3-methylmorpholin-4-yl]pyrimidin-2-yl]phenyl]-3-methylurea | 586 | 2.26 |
| 23k | 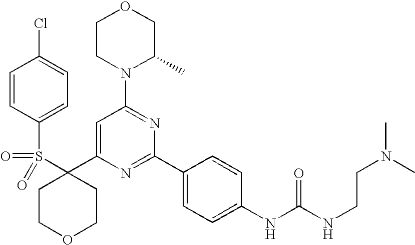 | 1-[4-[4-[4-(4-chlorophenyl)sulfonyloxan-4-yl]-6-[(3S)-3-methylmorpholin-4-yl]pyrimidin-2-yl]phenyl]-3-(2-dimethylaminoethyl)urea | 643 | 2.31 |
| 23l | 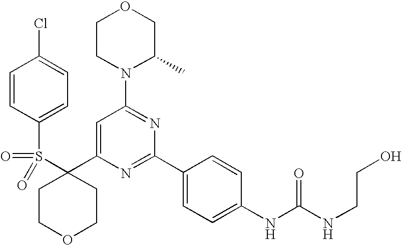 | 1-[4-[4-[4-(4-chlorophenyl)sulfonyloxan-4-yl]-6-[(3S)-3-methylmorpholin-4-yl]pyrimidin-2-yl]phenyl]-3-(2-hydroxyethyl)urea | 616 | 2.05 |
| 23m | 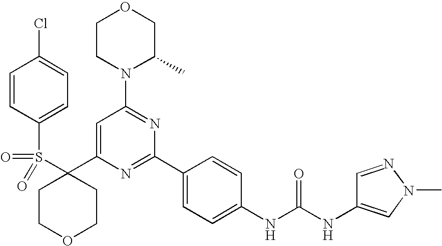 | 1-[4-[4-[4-(4-chlorophenyl)sulfonyloxan-4-yl]-6-[(3S)-3-methylmorpholin-4-yl]pyrimidin-2-yl]phenyl]-3-(1-methylpyrazol-4-yl)urea | 652 | 2.26 |

### EXAMPLE 23a

¹H NMR (399.902 MHz, CDCl₃) δ 1.35 (3H, d), 2.54 (2H, ddd), 2.71 (3H, s), 2.72 (2H, d), 2.89 (3H, d), 3.35 (1H, ddd), 3.41 (2H, dd), 3.62 (1H, ddd), 3.76 (1H, dd), 3.84 (1H, d), 4.0-4.1 (3H, m), 4.19 (1H, d), 4.46 (1H, br.d), 4.62 (1H, br.q), 6.32 (1H, s), 6.64 (1H, s), 7.40 (2H, d), 8.31 (2H, d).

mTOR Kinase Assay (Echo): 0.00459 μM

### EXAMPLE 23b

¹H NMR (399.902 MHz, CDCl₃) δ 1.35 (3H, d), 2.32 (6H, s), 2.5-2.6 (4H, m), 2.71 (3H, s), 2.72 (2H, d), 3.3-3.4 (3H, m), 3.41 (2H, ddd), 3.62 (1H, ddd), 3.76 (1H, dd), 3.84 (1H, d), 4.0-4.1 (3H, m), 4.19 (1H, d), 4.46 (1H, br.d), 5.22 (1H, br.t), 6.62 (1H, s), 7.44 (2H, d), 8.28 (2H, d).

mTOR Kinase Assay (Echo): 0.197 μM

### EXAMPLE 23c

¹H NMR (399.902 MHz, CDCl₃) δ 1.35 (3H, d), 2.5-2.6 (3H, m), 2.71 (3H, s), 2.72 (2H, d), 3.34 (1H, ddd), 3.41 (2H, dd), 3.45 (4H, dt), 3.62 (1H, ddd), 3.76 (1H, dd), 3.78 (2H, m), 3.84 (1H, d), 4.0-4.1 (3H, m), 4.19 (1H, d), 4.45 (1H, br.d), 5.25 (1H, t), 6.64 (1H, s), 6.86 (1H, s), 7.41 (2H, d), 8.30 (2H, d).

mTOR Kinase Assay (Echo): 0.000831 μM

### EXAMPLE 23d

¹H NMR (399.902 MHz, CDCl₃) δ 1.35 (3H, d), 2.54 (2H, ddd), 2.72 (3H, s), 2.72 (2H, m), 3.33 (1H, ddd), 3.41 (2H, dd), 3.61 (1H, ddd), 3.75 (1H, dd), 3.83 (1H, d), 3.90 (3H, s), 4.0-4.1 (3H, m), 4.18 (1H, d), 4.45 (1H, br.d), 6.25 (1H, br.s), 6.64 (1H, s), 6.78 (1H, s), 7.41 (1H, s), 7.44 (2H, d), 7.61 (1H, s), 8.29 (2H, d).

mTOR Kinase Assay (Echo): 0.00534 μM

### EXAMPLE 23e

¹H NMR (399.902 MHz, CDCl₃) δ 0.71 (2H, ddd), 0.83 (2H, m), 0.89 (2H, ddd), 1.02 (2H, m), 1.34 (3H, d), 2.18 (1H, tt), 2.53 (2H, ddd), 2.64 (1H, tt), 2.82 (2H, br.d), 3.34 (2H, ddd), 3.43 (3H, ddd), 3.63 (1H, ddd), 3.77 (1H, dd), 3.84 (1H, d), 4.02 (2H, m), 4.06 (1H, dd), 4.19 (1H, d), 4.46 (1H, br.d), 4.86 (1H, s), 6.67 (1H, s), 6.97 (1H, s), 7.51 (2H, d), 8.33 (2H, d).

mTOR Kinase Assay (Echo): 0.00876 μM

### EXAMPLE 23f

¹H NMR (399.902 MHz, CDCl₃) δ 0.83 (2H, m), 1.02 (2H, m), 1.34 (3H, d), 2.18 (1H, tt), 2.52 (2H, m), 2.82 (2H, br.d), 2.86 (3H, d), 3.33 (1H, ddd), 3.42 (2H, ddd), 3.62 (1H, ddd), 3.76 (1H, dd), 3.83 (1H, d), 4.01 (2H, m), 4.05 (1H, dd), 4.18 (1H, d), 4.45 (1H, br.d), 4.83 (1H, q), 6.63 (1H, s), 6.67 (1H, s), 7.40 (2H, d), 8.31 (2H, d).

mTOR Kinase Assay (Echo): 0.0199 μM

### EXAMPLE 23g

¹H NMR (399.902 MHz, CDCl₃) δ 0.82 (2H, m), 1.01 (2H, m), 1.34 (3H, d), 2.18 (1H, tt), 2.32 (6H, s), 2.52 (4H, m), 2.82 (2H, d), 3.32 (3H, m), 3.43 (2H, ddd), 3.62 (1H, ddd), 3.76 (1H, dd), 3.83 (1H, d), 4.01 (2H, m), 4.05 (2H, dd), 4.18 (1H, d), 4.46 (1H, br.d), 5.30 (1H, br.t), 6.66 (1H, s), 7.44 (2H, d), 8.30 (2H, d).

mTOR Kinase Assay (Echo): 1.21 μM

### EXAMPLE 23h

¹H NMR (399.902 MHz, CDCl₃) δ 0.83 (2H, m), 1.02 (2H, m), 1.34 (3H, d), 2.18 (1H, tt), 2.53 (3H, m), 2.82 (2H, br.d), 3.33 (1H, ddd), 3.43 (2H, dd), 3.46 (2H, t), 3.62 (1H, ddd), 3.76 (1H, dd), 3.78 (2H, t), 3.83 (1H, d), 4.01 (2H, m), 4.05 (1H, dd), 4.18 (1H, d), 4.45 (1H, br.d), 5.26 (1H, t), 6.67 (1H, s), 6.85 (1H, s), 7.40 (2H, d), 8.32 (2H, d).

mTOR Kinase Assay (Echo): 0.00808 μM

### EXAMPLE 23i

¹H NMR (399.902 MHz, CDCl₃) δ 0.84 (2H, m), 1.03 (2H, m), 1.34 (3H, d), 2.19 (1H, tt), 2.53 (2H, m), 2.82 (2H, br.d), 3.33 (1H, ddd), 3.42 (2H, ddd), 3.61 (1H, ddd), 3.76 (1H, dd), 3.83 (1H, d), 3.92 (3H, s), 4.02 (2H, m), 4.05 (1H, dd), 4.17 (1H, d), 4.45 (1H, br.d), 6.03 (1H, s), 6.60 (1H, s), 6.67 (1H, s), 7.42 (2H, d), 7.43 (1H, s), 7.60 (1H, s), 8.31 (2H, d).

mTOR Kinase Assay (Echo): 0.0254 μM

### EXAMPLE 23j

¹H NMR (399.902 MHz, CDCl₃) δ 1.36 (3H, d), 2.55 (2H, td), 2.64 (2H, br.d), 2.87 (3H, d), 3.30 (1H, ddd), 3.34 (1H, ddd), 3.65 (1H, ddd), 3.79 (1H, dd), 3.86 (1H, d), 3.99 (2H, m), 4.08 (1H, dd), 4.16 (1H, d), 4.45 (1H, br.d), 4.64 (1H, br.q), 6.29 (1H, s), 6.62 (1H, s), 7.27 (4H, m), 7.39 (2H, d), 7.83 (2H, d).

mTOR Kinase Assay (Echo): 0.0314 μM

### EXAMPLE 23k

¹H NMR (399.902 MHz, CDCl₃) δ 1.36 (3H, d), 2.32 (6H, s), 2.52 (2H, t), 2.55 (2H, ddd), 2.63 (2H, d), 3.26-3.37 (5H, m), 3.64 (1H, ddd), 3.79 (1H, dd), 3.85 (1H, d), 3.98 (2H, m), 4.07 (1H, dd), 4.17 (1H, d), 4.45 (1H, br.d), 5.18 (1H, br.t), 6.61 (1H, s), 7.27 (2H, d), 7.32 (2H, d), 7.39 (2H, d), 7.80 (2H, d).

mTOR Kinase Assay (Echo): 1.14 μM

### EXAMPLE 23l

¹H NMR (399.902 MHz, CDCl₃) δ 1.36 (3H, d), 2.37 (1H, t), 2.55 (4H, ddd), 2.64 (2H, br.d), 3.26-3.37 (3H, m), 3.46 (2H, dt), 3.64 (1H, ddd), 3.77-3.81 (3H, m), 3.86 (1H, d), 3.99 (2H, m), 4.08 (1H, dd), 4.16 (1H, d), 4.45 (1H, br.d), 5.07 (1H, t), 6.49 (1H, s), 6.63 (1H, s), 7.26 (2H, d), 7.28 (2H, d), 7.39 (2H, d), 7.84 (2H, d).

mTOR Kinase Assay (Echo): 0.00888 μM

### EXAMPLE 23m

¹H NMR (399.902 MHz, CDCl₃) δ 1.35 (3H, d), 2.53 (3H, ddd), 2.64 (2H, br.d), 3.26-3.37 (3H, m), 3.64 (1H, ddd), 3.78 (21H, dd), 3.85 (1H, d), 3.91 (3H, s), 3.99 (2H, m), 4.07 (1H, dd), 4.15 (1H, d), 4.44 (1H, br.d), 6.11 (1H, s), 6.61 (1H, s), 6.63 (1H, s), 7.28 (2H, d), 7.32 (2H, d), 7.41 (2H, d), 7.42 (1H, s), 7.61 (1H, s), 7.84 (2H, d).

mTOR Kinase Assay (Echo): 0.0577 μM

The preparation of phenyl N-[4-[4-[(3S)-3-methylmorpholin-4-yl]-6-(4-methylsulfonyloxan-4-yl)pyrimidin-2-yl]phenyl]carbamate is described below.

### Phenyl N-[4-[4-[(3S)-3-methylmorpholin-4-yl]-6-(4-methylsulfonyloxan-4-yl)pyrimidin-2-yl]phenyl]carbamate

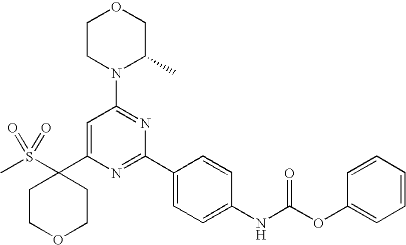

Phenyl chloroformate (0.196 mL, 1.56 mmol) was added dropwise to 4-[4-[(3S)-3-methylmorpholin-4-yl]-6-(4-methylsulfonyloxan-4-yl)pyrimidin-2-yl]aniline (615 mg, 1.42 mmol) and sodium hydrogen carbonate (179 mg, 2.13 mmol) in dioxane (50 mL) at RT. The resulting suspension was stirred at RT for 2 hours. The reaction mixture was concentrated, diluted with ethyl acetate (100 mL) and washed sequentially with water (2×100 mL) and saturated brine (50 mL). The organic layer was dried (MgSO₄), filtered and evaporated. The crude product was purified by flash silica chromatography, elution gradient 40 to 80% ethyl acetate in iso-hexane, to afford the desired material as a white solid (714 mg).

NMR Spectrum: ¹H NMR (399.902 MHz, CDCl₃) δ 1.36 (3H, d), 2.55 (2H, m), 2.71 (3H, s), 2.73 (2H, d), 3.35 (1H, ddd), 3.42 (2H, ddd), 3.62 (1H, ddd), 3.77 (1H, dd), 3.84 (1H, d), 4.02-4.08 (3H, m), 4.20 (1H, d), 4.47 (1H, br.d), 6.66 (1H, s), 7.11 (1H, s), 7.21 (2H, d), 7.26 (1H, dd), 7.41 (2H, dd), 7.54 (2H, d), 8.35 (2H, d).

LCMS Spectrum: m/z (ESI+)(M+H)+=553; HPLC tR=2.57 min.

### 4-[4-[(3S)-3-Methylmorpholin-4-yl]-6-(4-methylsulfonyloxan-4-yl)pyrimidin-2-yl]aniline

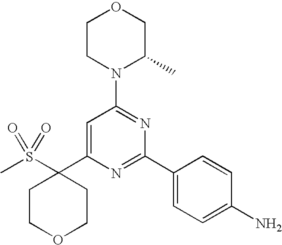

Sodium carbonate (2M in water, 5.75 mL, 11.49 mmol) was added to 4-(4,4,5,5-tetramethyl-1,3,2-dioxaborolan-2-yl)aniline (0.839 g, 3.83 mmol) and 2-chloro-4-[(3S)-3-methylmorpholin-4-yl]-6-(4-methylsulfonyloxan-4-yl)pyrimidine (1.200 g, 3.19 mmol) in a mixture of ethylene glycol diethyl ether (10 mL), ethanol (10 mL), DMF (10 mL) and water (20 mL) at RT under nitrogen. The mixture was degassed and purged with nitrogen. Bis(triphenylphosphine)palladium(II) chloride (0.112 g, 0.16 mmol) was added and the mixture was degassed and purged with nitrogen. The resulting suspension was stirred under nitrogen at 80° C. for 90 minutes. The reaction mixture was concentrated and diluted with ethyl acetate (150 mL) and washed sequentially with water (2×150 mL) and saturated brine (100 mL). The organic layer was dried (MgSO₄), filtered and evaporated. The crude product was purified by flash silica chromatography, elution gradient 40 to 100% ethyl acetate in iso-hexane, to afford the desired material as a white solid (690 mg).

NMR Spectrum: ¹H NMR (399.902 MHz, CDCl₃) δ 1.34 (3H, d), 2.53 (2H, ddd), 2.70 (3H, s), 2.72 (2H, br.d), 3.33 (1H, ddd), 3.41 (2H, ddd), 3.61 (1H, ddd), 3.76 (1H, dd), 3.83 (1H, d), 3.93 (2H, s), 4.03 (3H, m), 4.18 (1H, d), 4.45 (1H, br.d), 6.58 (1H, s), 6.71 (2H, d), 8.18 (2H, d).

LCMS Spectrum: m/z (ESI+)(M+H)+=433; HPLC tR=1.98 min.

### 2-Chloro-4-[(3S)-3-methylmorpholin-4-yl]-6-(4-methylsulfonyloxan-4-yl)pyrimidine

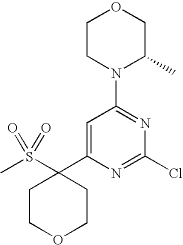

Sodium tert-butoxide (1.38 g, 14.39 mmol) was added portionwise to a mixture of 2-chloro-4-[(3S)-3-methylmorpholin-4-yl]-6-(methylsulfonylmethyl)pyrimidine (2.00 g, 6.54 mmol) and bis(2-bromoethyl) ether (2.055 mL, 16.35 mmol) in DMF (75 mL) at 0° C. over a period of 10 minutes under nitrogen. The resulting solution was allowed to warm to RT and stirred for 7 hours. Further sodium tert-butoxide (629 mg, 6.54 mmol) was added portionwise and the solution was stirred at RT for a further 45 hours. The reaction mixture was concentrated, diluted with ethyl acetate (200 mL) and washed sequentially with water (2×200 mL) and saturated brine (100 mL). The organic layer was dried (MgSO₄), filtered and evaporated. The crude product was purified by flash silica chromatography, elution gradient 40 to 100% ethyl acetate in iso-hexane. Pure fractions were evaporated to dryness and the residue crystallised from ethyl acetate/iso-hexane to afford the desired material as a white crystalline solid (1.42 g).

NMR Spectrum: ¹H NMR (399.902 MHz, CDCl₃) δ 1.34 (3H, d), 2.50 (2H, m), 2.55 (2H, m), 2.73 (3H, s), 3.33 (3H, m), 3.56 (1H, ddd), 3.71 (1H, dd), 3.80 (1H, d), 4.01 (4H, m), 4.31 (1H, br.s), 6.62 (1H, s).

LCMS Spectrum: m/z (ESI+)(M+H)+=376, 378; HPLC tR=1.85 min.

The preparation of 2-chloro-4-[(3S)-3-methylmorpholin-4-yl]-6-(methylsulfonylmethyl)pyrimidine was described earlier.

The preparation of phenyl N-[4-[4-(4-cyclopropylsulfonyloxan-4-yl)-6-[(3S)-3-methylmorpholin-4-yl]pyrimidin-2-yl]phenyl]carbamate is described below.

### Phenyl N-[4-[4-(4-cyclopropylsulfonyloxan-4-yl)-6-[(3S)-3-methylmorpholin-4-yl]pyrimidin-2-yl]phenyl]carbamate

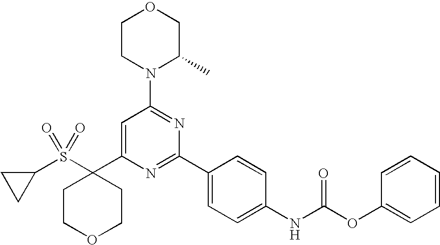

Phenyl chloroformate (0.211 mL, 1.68 mmol) was added to 4-[4-(4-cyclopropylsulfonyloxan-4-yl)-6-[(3S)-3-methylmorpholin-4-yl]pyrimidin-2-yl]aniline (700 mg, 1.53 mmol) and sodium hydrogen carbonate (192 mg, 2.29 mmol) in 1,4-dioxane (50 mL) at RT. The resulting suspension was stirred at RT for 16 hours. The reaction mixture was concentrated and diluted with ethyl acetate (75 mL) and washed sequentially with water (75 mL) and saturated brine (50 mL). The organic layer was dried (MgSO₄), filtered and evaporated. The crude product was purified by flash silica chromatography, elution gradient 30 to 70% ethyl acetate in iso-hexane, to afford the desired material as a white solid (850 mg).

NMR Spectrum: ¹H NMR (399.902 MHz, CDCl₃) δ 0.83 (2H, m), 1.02 (2H, m), 1.35 (3H, d), 2.18 (1H, tt), 2.54 (2H, m), 2.83 (2H, d), 3.34 (2H, ddd), 3.43 (2H, dd), 3.63 (1H, ddd), 3.77 (1H, dd), 3.84 (1H, d), 4.02 (2H, m), 4.06 (1H, dd), 4.20 (1H, d), 4.47 (1H, br.d), 6.69 (1H, s), 7.08 (1H, s), 7.21 (2H, d), 7.26 (1H, dd), 7.41 (2H, dd), 7.54 (2H, d), 8.37 (2H, d).

LCMS Spectrum: m/z (ESI+)(M+H)+=579.5; HPLC tR=2.72 min.

### 4-[4-(4-Cyclopropylsulfonyloxan-4-yl)-6-[(3S)-3-methylmorpholin-4-yl]pyrimidin-2-yl]aniline

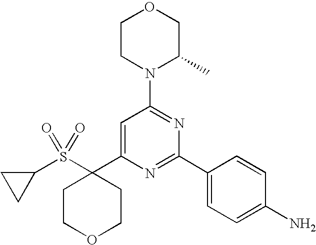

Sodium carbonate (2M in water, 7.48 mL, 14.96 mmol) was added to 4-(4,4,5,5-tetramethyl-1,3,2-dioxaborolan-2-yl)aniline (1.092 g, 4.99 mmol) and 2-chloro-4-(4-cyclopropylsulfonyloxan-4-yl)-6-[(3S)-3-methylmorpholin-4-yl]pyrimidine (1.67 g, 4.16 mmol) in a mixture of ethylene glycol diethyl ether (10 mL), ethanol (10 mL), DMF (10 mL) and water (20 mL) at RT under nitrogen. The mixture was degassed and purged with nitrogen. Bis(triphenylphosphine)palladium(II) chloride (0.146 g, 0.21 mmol) was added and the mixture was degassed and purged with nitrogen. The resulting suspension was stirred under nitrogen at 80° C. for 90 minutes. The reaction mixture was concentrated and diluted with ethyl acetate (150 mL) and washed sequentially with water (2×150 mL) and saturated brine (100 mL). The organic layer was dried (MgSO₄), filtered and evaporated. The crude product was purified by flash silica chromatography, elution gradient 40 to 70% ethyl acetate in iso-hexane, to afford the desired material as a white solid (740 mg).

NMR Spectrum: ¹H NMR (399.902 MHz, CDCl₃) δ 0.82 (2H, m), 1.01 (2H, m), 1.33 (3H, d), 2.17 (1H, tt), 2.51 (2H, m), 2.82 (2H, br.d), 3.32 (1H, ddd), 3.43 (2H, ddd), 3.62 (1H, ddd), 3.76 (1H, dd), 3.83 (1H, d), 3.91 (2H, s), 3.98 (2H, m), 4.05 (1H, dd), 4.17 (1H, d), 4.45 (1H, br.d), 6.62 (1H, s), 6.71 (2H, d), 8.21 (2H, d).

LCMS Spectrum: m/z (ESI+)(M+H)+=459; HPLC tR=2.05 min.

### 2-Chloro-4-(4-cyclopropylsulfonyloxan-4-yl)-6-[(3S)-3-methylmorpholin-4-yl]pyrimidine

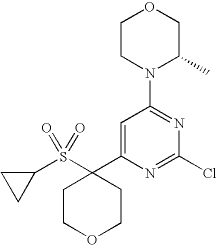

Sodium tert-butoxide (1.738 g, 18.08 mmol) was added portionwise to 2-chloro-4-(cyclopropylsulfonylmethyl)-6-[(3S)-3-methylmorpholin-4-yl]pyrimidine (2.00 g, 6.03 mmol) and bis(2-bromoethyl) ether (2.273 mL, 18.08 mmol) in DMF (75 mL) at RT over a period of 5 minutes under nitrogen. The resulting solution was stirred at RT for 5 hours. Further bis(2-bromoethyl) ether (0.758 mL, 6.03 mmol), and sodium tert-butoxide (0.579 g, 6.03 mmol) were added and the solution was stirred at RT for a further 20 hours. The reaction mixture was quenched with saturated aqueous ammonium chloride solution (0.5 mL), concentrated, diluted with ethyl acetate (200 mL) and washed sequentially with water (2×200 mL) and saturated brine (100 mL). The organic layer was dried (MgSO₄), filtered and evaporated to afford crude product. The crude product was purified by flash silica chromatography, elution gradient 40 to 60% ethyl acetate in iso-hexane, to afford the desired material as a colourless oil which crystallised on standing (1.734 g).

NMR Spectrum: ¹H NMR (399.902 MHz, CDCl₃) δ 1.01 (2H, m), 1.01 (2H, m), 1.33 (3H, d), 2.22 (1H, tt), 2.47 (2H, ddd), 2.64 (2H, br.d), 3.30 (1H, ddd), 3.36 (2H, dd), 3.56 (1H, ddd), 3.71 (1H, dd), 3.80 (1H, d), 3.97-4.04 (4H, m), 4.30 (1H, br.d), 6.66 (1H, s).

LCMS Spectrum: m/z (ESI−)(M−H)−=400.4; HPLC tR=2.04 min.

The preparation of 2-chloro-4-(cyclopropylsulfonylmethyl)-6-[(3S)-3-methylmorpholin-4-yl]pyrimidine was described earlier.

The preparation of phenyl N-[4-[4-[4-(4-chlorophenyl)sulfonyloxan-4-yl]-6-[(3S)-3-methylmorpholin-4-yl]pyrimidin-2-yl]phenyl]carbamate is described below.

### Phenyl N-[4-[4-[4-(4-chlorophenyl)sulfonyloxan-4-yl]-6-[(3S)-3-methylmorpholin-4-yl]pyrimidin-2-yl]phenyl]carbamate

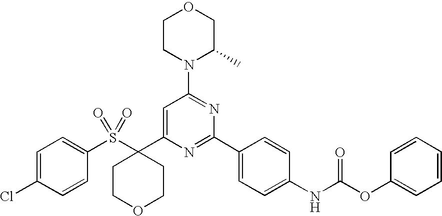

Phenyl chloroformate (0.198 mL, 1.58 mmol) was added to 4-[4-[4-(4-chlorophenyl)sulfonyloxan-4-yl]-6-[(3S)-3-methylmorpholin-4-yl]pyrimidin-2-yl]aniline (759 mg, 1.43 mmol) and sodium hydrogen carbonate (181 mg, 2.15 mmol) in 1,4-dioxane (50 mL) at RT. The resulting suspension was stirred at RT for 16 hours. The reaction mixture was concentrated and diluted with ethyl acetate (75 mL) and washed sequentially with water (75 mL) and saturated brine (50 mL). The organic layer was dried (MgSO₄), filtered and evaporated to afford crude product. The crude product was purified by flash silica chromatography, elution gradient 30 to 70% ethyl acetate in iso-hexane, to afford the desired material as a white dry film (780 mg).

NMR Spectrum: ¹H NMR (399.902 MHz, CDCl₃) δ 1.36 (3H, d), 2.56 (2H, ddd), 2.64 (2H, br.d), 3.31 (2H, ddd), 3.35 (1H, ddd), 3.65 (1H, ddd), 3.80 (1H, dd), 3.86 (1H, d), 4.00 (2H, m), 4.08 (1H, dd), 4.18 (1H, d), 4.45 (1H, br.d), 6.64 (1H, s), 7.04 (1H, s), 7.21 (2H, d), 7.22 (1H, dd), 7.27 (2H, d), 7.39 (2H, d), 7.40 (2H, dd), 7.43 (2H, d), 7.87 (2H, d).

LCMS Spectrum: m/z (ESI+)(M+H)+=649, 651; HPLC tR=3.02 min.

### 4-[4-[4-(4-Chlorophenyl)sulfonyloxan-4-yl]-6-[(3S)-3-methylmorpholin-4-yl]pyrimidin-2-yl]aniline

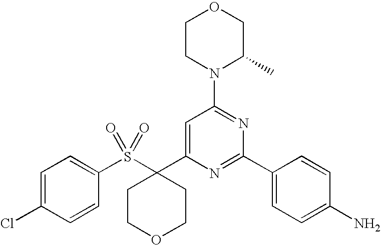

Sodium carbonate (2M in water, 4.02 mL, 8.05 mmol) was added to 4-(4,4,5,5-tetramethyl-1,3,2-dioxaborolan-2-yl)aniline (490 mg, 2.24 mmol) and 2-chloro-4-[4-(4-chlorophenyl)sulfonyloxan-4-yl]-6-[(3S)-3-methylmorpholin-4-yl]pyrimidine (1056 mg, 2.24 mmol) in a mixture of ethanol (10 mL), water (20 mL), DMF (10 mL) and ethylene glycol diethyl ether (10 mL) at RT under nitrogen. The mixture was degassed and purged with nitrogen. Bis(triphenylphosphine)palladium(II) chloride (78 mg, 0.11 mmol) was added and the mixture degassed and purged with nitrogen. The resulting suspension was stirred at 80° C. for 2 hours. The reaction mixture was concentrated and diluted with ethyl acetate (100 mL) and water (100 mL). The resulting precipitate was removed by filtration. The organic layer was washed sequentially with water (100 mL) and saturated brine (100 mL), dried (MgSO₄), filtered and evaporated. The crude product was purified by flash silica chromatography, elution gradient 30 to 50% ethyl acetate in iso-hexane, to afford the desired material as a white dry film (790 mg).

NMR Spectrum: ¹H NMR (399.902 MHz, CDCl₃) δ 1.35 (3H, d), 2.54 (2H, ddd), 2.63 (2H, d), 3.27-3.35 (3H, m), 3.64 (1H, ddd), 3.79 (1H, dd), 3.85 (1H, d), 3.87 (2H, s), 3.98 (2H, m), 4.06 (1H, dd), 4.16 (1H, d), 4.44 (1H, br.d), 6.56 (1H, s), 6.60 (2H, d), 7.27 (2H, d), 7.39 (2H, d), 7.69 (2H, d).

LCMS Spectrum: m/z (ESI+)(M+H)+=529.5, 531.5; HPLC tR=2.49 min.

### 2-Chloro-4-[4-(4-chlorophenyl)sulfonyloxan-4-yl]-6-[(3S)-3-methylmorpholin-4-yl]pyrimidine

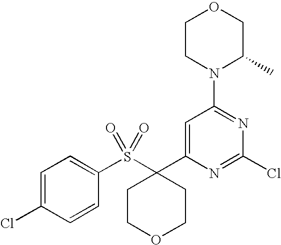

Sodium tert-butoxide (1.566 g, 16.30 mmol) was added portionwise to 2-chloro-4-[(4-chlorophenyl)sulfonylmethyl]-6-[(3S)-3-methylmorpholin-4-yl]pyrimidine (1.873 g, 4.66 mmol) and bis(2-bromoethyl) ether (1.463 mL, 11.64 mmol) in DMF (75 mL) at RT over a period of 5 minutes under nitrogen. The resulting solution was stirred at RT for 6 hours. Further sodium tert-butoxide (0.895 g, 9.31 mmol) was added and the solution was stirred at RT for a further 4 days. The reaction mixture was concentrated and diluted with ethyl acetate (200 mL), and washed sequentially with water (2×200 mL) and saturated brine (100 mL). The organic layer was dried (MgSO₄), filtered and evaporated. The crude product was purified by flash silica chromatography, elution gradient 40 to 50% ethyl acetate in iso-hexane, to afford the desired material as a white dry film (1.0 g).

NMR Spectrum: ¹H NMR (399.902 MHz, CDCl₃) δ 1.35 (3H, d), 2.45-2.49 (4H, m), 3.22-3.35 (3H, m), 3.59 (1H, ddd), 3.73 (1H, dd), 3.82 (1H, d), 3.95-4.00 (3H, m), 4.04 (1H, dd), 4.31 (1H, br.s), 6.67 (1H, s), 7.39 (2H, d), 7.45 (2H, d).

LCMS Spectrum: m/z (ESI−)(M−H)−=470, 472; HPLC tR=2.62 min.

### 2-Chloro-4-[(4-chlorophenyl)sulfonylmethyl]-6-[(3S)-3-methylmorpholin-4-yl]pyrimidine

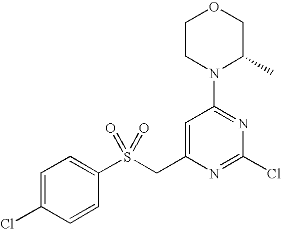

4-Chlorobenzenesulphinic acid sodium salt (5.39 g, 27.15 mmol) was added in one portion to 2-chloro-4-(iodomethyl)-6-[(3S)-3-methylmorpholin-4-yl]pyrimidine (8.00 g, 22.63 mmol) in acetonitrile (400 mL) at RT. The resulting suspension was stirred at 85° C. under reflux for 5 hours. The reaction mixture was concentrated and diluted with DCM (400 mL) and washed with water (400 mL). The organic layer was dried (MgSO₄), filtered and evaporated. The crude product was purified by flash silica chromatography, elution gradient 25 to 40% ethyl acetate in isohexane, to give the desired material as a white solid (6.90 g).

NMR Spectrum: ¹H NMR (399.902 MHz, CDCl3) δ 1.33 (3H, d), 3.30 (1H, ddd), 3.55 (1H, ddd), 3.70 (1H, dd), 3.80 (1H, d), 4.02 (2H, m), 4.28 (1H, br.s), 4.29 (2H, s), 6.55 (1H, s), 7.51 (2H, d), 7.70 (2H, d).

LCMS Spectrum: m/z (ESI+)(M+H)+=402, 404; HPLC tR=2.26 min.

The preparation of 2-chloro-4-(iodomethyl)-6-[(3S)-3-methylmorpholin-4-yl]pyrimidine was described earlier.

### EXAMPLE 24

### 1-[4-[4-[4-(4-Chlorophenyl)sulfonyloxan-4-yl]-6-[(3S)-3-methylmorpholin-4-yl]pyrimidin-2-yl]phenyl]-3-cyclopropylurea

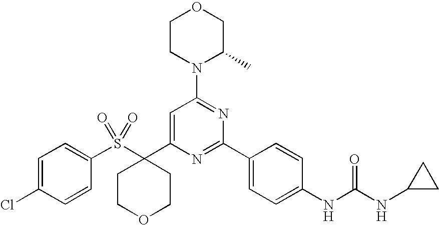

Sodium carbonate (0.381 mL, 0.76 mmol) was added to 2-chloro-4-[4-(4-chlorophenyl)sulfonyloxan-4-yl]-6-[(3S)-3-methylmorpholin-4-yl]pyrimidine (100 mg, 0.21 mmol) and 4-(3-cyclopropylureido)phenylboronic acid (47 mg, 0.21 mmol) in DME (2 mL), ethanol (2 mL), DMF (2 mL) and water (4 mL) at RT under nitrogen. The mixture was degassed and purged with nitrogen then bis(triphenylphosphine)palladium(II) chloride (7.4 mg, 0.011 mmol) added and the mixture stirred at 85° C. for 2 hours. The reaction mixture was concentrated, diluted with ethyl acetate (25 mL) and washed sequentially with water (2×25 mL) and saturated brine (25 mL). The organic layer was dried (MgSO₄), filtered and evaporated. The crude product was purified by flash silica chromatography, elution gradient 30 to 50% ethyl acetate in isohexane. The isolated material was further purified trituration with diethyl ether to afford the desired material as a white solid (26 mg).

NMR Spectrum: ¹H NMR (400.132 MHz, CDCl₃) δ 0.70 (2H, m), 0.88 (2H, m), 1.36 (3H, d), 2.58 (5H, m), 3.31 (2H, m), 3.34 (1H, ddd), 3.65 (1H, ddd), 3.79 (1H, dd), 3.86 (1H, d), 3.99 (2H, m), 4.08 (1H, dd), 4.17 (1H, d), 4.45 (1H, d), 4.88 (1H, s), 6.63 (1H, s), 6.94 (1H, s), 7.27 (2H, d), 7.39 (2H, d), 7.39 (2H, d), 7.84 (2H, d).

LCMS Spectrum: m/z (ESI+)(M+H)+=612, 6144; HPLC tR=2.48 min.

mTOR Kinase Assay (Echo): 0.00517 μM

The preparation of 2-chloro-4-[4-(4-chlorophenyl)sulfonyloxan-4-yl]-6-[(3S)-3-methylmorpholin-4-yl]pyrimidine was described earlier.

### EXAMPLE 25

### 3-Cyclopropyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea

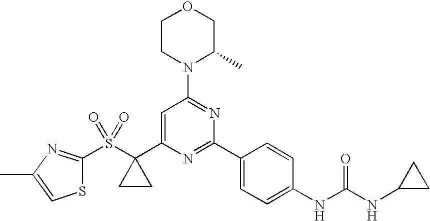

To cyclopropylamine (57 mg, 1 mmol) was added a solution of phenyl N-[4-[4-[(3S)-3-methylmorpholin-4-yl]-6-[1-[(4-methyl-1,3-thiazol-2-yl)sulfonyl]cyclopropyl]pyrimidin-2-yl]phenyl]carbamate (100 mg, 0.17 mmol) in DMF (1.5 mL). Triethylamine (0.082 mL, 0.59 mmol) was then added and the resultant mixture was heated to 50° C. and stirred overnight (˜18 hours). The reaction mixture was cooled then purified by preparative HPLC, using decreasingly polar mixtures of water (containing 1% ammonia) in acetonitrile as eluents, to give the desired material as a white solid (50 mg).

NMR Spectrum: ¹H NMR (400.132 MHz, DMSO-d₆) δ 0.40-0.43 (2H, m), 0.62-0.67 (2H, m), 1.21 (3H, d), 1.75-1.80 (2H, m), 1.93-1.97 (2H, m), 2.47-2.49 (3H, m), 2.51-2.58 (1H, m), 3.17 (1H, td), 3.47 (1H, td), 3.62 (1H, dd), 3.76 (1H, d), 3.97 (1H, dd), 4.10-4.20 (1H, m), 4.41-4.48 (1H, m), 6.41 (1H, d), 6.77 (1H, s), 7.41-7.44 (2H, m), 7.83 (1H, d), 7.87-7.90 (2H, m), 8.52 (1H, s).

LCMS Spectrum: m/z (ESI+)(M+H)+=555; HPLC tR=2.13 min.

mTOR Kinase Assay (Echo): 0.00155 μM

The compounds below were prepared in an analogous fashion from phenyl N-[4-[4-[(3S)-3-methylmorpholin-4-yl]-6-[1-[(4-methyl-1,3-thiazol-2-yl)sulfonyl]cyclopropyl]pyrimidin-2-yl]phenyl]carbamate using the appropriate amine.

| | | | LCMS | Retention |
|---|---|---|---|---|
| Example | Structure | NAME | MH+ | time (min) |
| 25a | 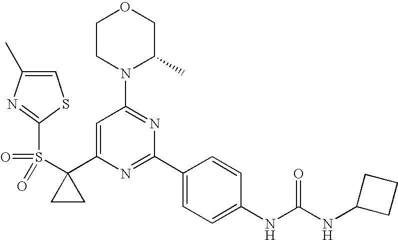 | 3-cyclobutyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 569.6 | 1.90 |
| 25b | 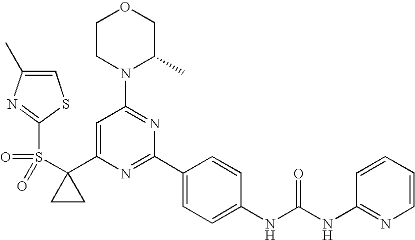 | 1-[4-[4-[(3S)-3-methylmoprholin-4-yl]-6-[1-[(4-methyl-1,3-thiazol-2-yl)sulfonyl]cyclopropyl]pyrimidin-2-yl]phenyl]-3-pyridin-2-ylurea | 592.4 | 2.51 |
| 25c | 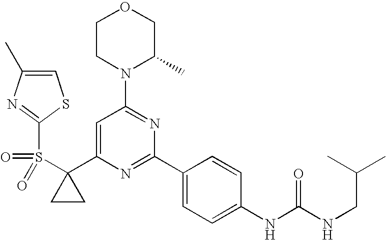 | 1-[4-[4-[(3S)-3-methylmorpholin-4-yl]-6-[1-[(4-methyl-1,3-thiazol-2-yl)sulfonyl]cyclopropyl]pyrimidin-2-yl]phenyl]-3-(2-methylpropyl)urea | 571.4 | 2.42 |
| 25d | 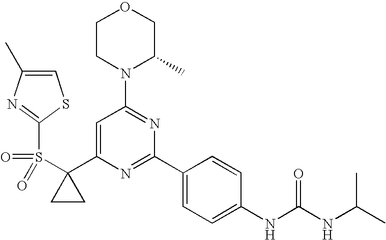 | 1-[4-[4-[(3S)-3-methylmorpholin-4-yl]-6-[1-[(4-methyl-1,3-thiazol-2-yl)sulfonyl]cyclopropyl]pyrimidin-2-yl]phenyl]-3-propan-2-ylurea | 557.4 | 2.26 |
| 25e | 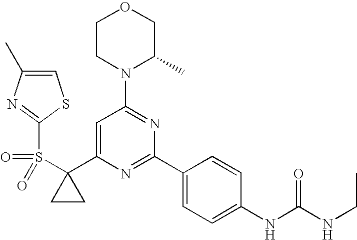 | 1-ethyl-3-[4-[4-[(3S)-3-methylmorpholin-4-yl]-6-[1-[(4-methyl-1,3-thiazol-2-yl)sulfonyl]cyclpropyl]pyrimidin-2-yl]phenyl]urea | 543.4 | 2.08 |
| 25f | 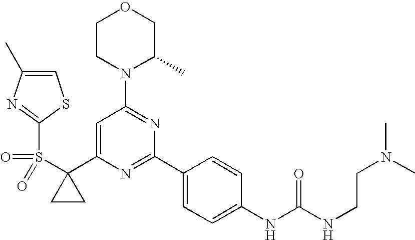 | 3-(2-dimethylaminoethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 586.4 | 1.98 |
| 25g | 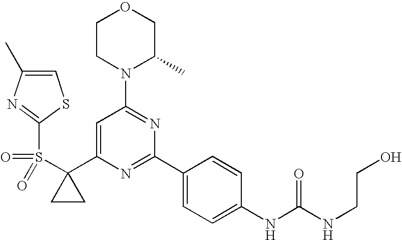 | 3-(2-hydroxyethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 559.3 | 1.78 |
| 25h | 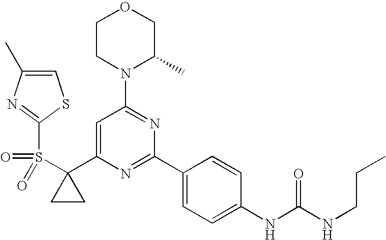 | 3-[4-[4-[(3S)-3-methylmorpholin-4-yl]-6-[1-[(4-methyl-1,3-thiazol-2-yl)sulfonyl]cyclopropyl]pyrimidin-2-yl]phenyl]-1-propylurea | 557.4 | 2.26 |
| 25i | 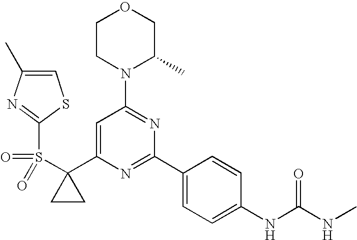 | 3-methyl-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 529.3 | 1.94 |
| 25j | 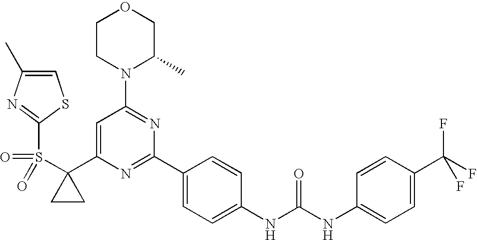 | 1-[4-[4-[(3S)-3-methylmorpholin-4-yl]-6-[1-[(4-methyl-1,3-thiazol-2-yl)sulfonyl]cyclopropyl]pyrimidin-2-yl]phenyl]-3-[4-(trifluoromethyl)phenyl]urea | 659.3 | 2.9 |
| 25k | 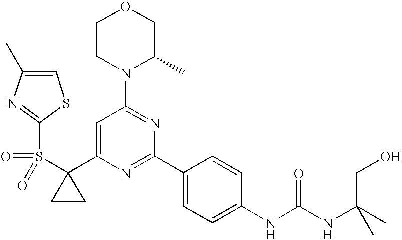 | 3-(1-hydroxy-2-methylpropan-2-yl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 587.4 | 2.05 |
| 25l | 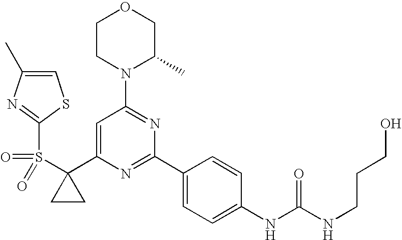 | 3-(3-hydroxypropyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 573.4 | 1.82 |
| 25m | 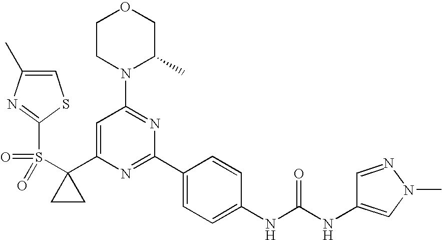 | 1-[4-[4-[(3S)-3-methylmorpholin-4-yl]-6-[1-[(4-methyl-1,3-thiazol-2-yl)sulfonyl]cyclopropyl]pyrimidin-2-yl]phenyl]-3-(1-methylpyrazol-4-yl)urea | 595.4 | 1.99 |

### EXAMPLE 25a

¹H NMR (400.132 MHz, DMSO-d₆) δ 1.20 (3H, d), 1.56-1.67 (2H, m), 1.75-1.79 (2H, m), 1.82-1.88 (2H, m), 1.93-1.95 (2H, m), 2.16-2.24 (2H, m), 2.48 (3H, s), 3.17 (1H, td), 3.46 (1H, td), 3.61 (1H, dd), 3.76 (1H, d), 3.97 (1H, dd), 4.08-4.20 (2H, m), 4.41-4.49 (1H, m), 6.45 (1H, d), 6.77 (1H, s), 7.39 (2H, d), 7.83-7.88 (3H, m), 8.57 (1H, s).

mTOR Kinase Assay (Echo): 0.00224 μM

### EXAMPLE 25b

¹H NMR (400.132 MHz, DMSO-d₆) δ 1.22 (3H, d), 1.78-1.82 (2H, m), 1.95-1.99 (2H, m), 2.49-2.50 (3H, m), 3.20 (1H, td), 3.48 (1H, td), 3.63 (1H, dd), 3.77 (1H, d), 3.98 (1H, dd), 4.13-4.21 (1H, m), 4.43-4.50 (1H, m), 6.81 (1H, s), 7.02-7.05 (1H, m), 7.53-7.62 (3H, m), 7.75-7.79 (1H, m), 7.85-7.87 (1H, m), 7.96-7.99 (2H, m), 8.29-8.31 (1H, m), 9.42 (1H, s), 10.53 (1H, s).

mTOR Kinase Assay (Echo): 0.000817 μM

### EXAMPLE 25c

¹H NMR (400.132 MHz, DMSO-d₆) δ 0.89 (6H, d), 1.21 (3H, d), 1.65-1.75 (1H, m), 1.75-1.80 (2H, m), 1.93-1.98 (2H, m), 2.48-2.49 (3H, m), 2.94 (2H, t), 3.18 (1H, td), 3.47 (1H, td), 3.62 (1H, dd), 3.76 (1H, d), 3.97 (1H, dd), 4.12-4.20 (1H, m), 4.41-4.49 (1H, m), 6.22 (1H, t), 6.77 (1H, s), 7.39-7.42 (2H, m), 7.83-7.84 (1H, m), 7.87-7.90 (2H, m), 8.62 (1H, s).

mTOR Kinase Assay (Echo): 0.00385 μM

### EXAMPLE 25d

¹H NMR (400.132 MHz, DMSO-d₆) δ 1.11 (6H, d), 1.21 (3H, d), 1.76-1.80 (2H, m), 1.93-1.97 (2H, m), 2.48-2.49 (3H, m), 3.18 (1H, td), 3.47 (1H, td), 3.62 (1H, dd), 3.73-3.81 (2H, m), 3.97 (1H, dd), 4.12-4.19 (1H, m), 4.41-4.48 (1H, m), 6.03 (1H, d), 6.77 (1H, s), 7.38-7.41 (2H, m), 7.84-7.84 (1H, m), 7.87-7.90 (2H, m), 8.51 (1H, s).

mTOR Kinase Assay (Echo): 0.00157 μM

### EXAMPLE 25e

¹H NMR (400.132 MHz, DMSO-d₆) δ 1.07 (3H, t), 1.21 (3H, d), 1.76-1.80 (2H, m), 1.94-1.97 (2H, m), 2.48-2.49 (3H, m), 3.09-3.21 (3H, m), 3.47 (1H, td), 3.62 (1H, dd), 3.76 (1H, d), 3.97 (1H, dd), 4.12-4.19 (1H, m), 4.41-4.49 (1H, m), 6.15 (1H, t), 6.77 (1H, s), 7.39-7.43 (2H, m), 7.83-7.84 (1H, m), 7.86-7.90 (2H, m), 8.65 (1H, s).

mTOR Kinase Assay (Echo): 0.000277 μM

### EXAMPLE 25f

¹H NMR (400.132 MHz, DMSO-d₆) δ 1.26 (3H, d), 1.81-1.85 (2H, m), 1.99-2.02 (2H, m), 2.23 (6H, s), 2.39 (2H, t), 2.53-2.54 (3H, m), 3.19-3.27 (3H, m), 3.52 (1H, td), 3.67 (1H, dd), 3.81 (1H, d), 4.02 (1H, dd), 4.17-4.24 (1H, m), 4.47-4.53 (1H, m), 6.20 (1H, t), 6.82 (1H, s), 7.44-7.47 (2H, m), 7.88-7.90 (1H, m), 7.92-7.95 (2H, m), 8.92 (1H, s).

mTOR Kinase Assay (Echo): 0.0547 μM

### EXAMPLE 25g

¹H NMR (400.132 MHz, DMSO-d₆) δ 1.21 (3H, d), 1.76-1.80 (2H, m), 1.93-1.97 (2H, m), 2.47-2.48 (3H, m), 3.13-3.21 (3H, m), 3.43-3.50 (3H, m), 3.62 (1H, dd), 3.76 (1H, d), 3.97 (1H, dd), 4.12-4.19 (1H, m), 4.41-4.49 (1H, m), 4.73 (1H, t), 6.23 (1H, t), 6.77 (1H, s), 7.39-7.42 (2H, m), 7.83-7.84 (1H, m), 7.87-7.90 (2H, m), 8.78 (1H, s).

mTOR Kinase Assay (Echo): 0.00119 μM

### EXAMPLE 25h

¹H NMR (400.132 MHz, DMSO-d₆) δ 0.89 (3H, t), 1.21 (3H, d), 1.46 (2H, sextet), 1.76-1.80 (2H, m), 1.94-1.97 (2H, m), 2.48-2.49 (3H, m), 3.06 (2H, q), 3.14-3.21 (1H, m), 3.47 (1H, td), 3.62 (1H, dd), 3.76 (1H, d), 3.97 (1H, dd), 4.12-4.19 (1H, m), 4.41-4.49 (1H, m), 6.19 (1H, t), 6.77 (1H, s), 7.39-7.43 (2H, m), 7.83-7.84 (1H, m), 7.86-7.90 (2H, m), 8.63 (1H, s).

mTOR Kinase Assay (Echo): 0.000993 μM

### EXAMPLE 25i

¹H NMR (400.132 MHz, DMSO-d₆) δ 1.21 (3H, d), 1.76-1.80 (2H, m), 1.94-1.97 (2H, m), 2.48-2.49(3H, m), 2.66 (3H, d), 3.18 (1H, td), 3.47 (1H, td), 3.62 (1H, dd), 3.76 (1H, d), 3.97 (1H, dd), 4.11-4.20 (1H, m), 4.40-4.50 (1H, m), 6.05 (1H, q), 6.77 (1H, s), 7.40-7.44 (2H, m), 7.83-7.84 (1H, m), 7.86-7.90 (2H, m), 8.72 (1H, s).

mTOR Kinase Assay (Echo): 0.001 μM

### EXAMPLE 25j

¹H NMR (400.132 MHz, DMSO-d₆) δ 1.22 (3H, d), 1.77-1.81 (2H, m), 1.95-1.99 (2H, m), 2.48-2.49 (3H, m), 3.19 (1H, td), 3.48 (1H, td), 3.63 (1H, dd), 3.77 (1H, d), 3.98 (1H, dd), 4.13-4.21 (1H, m), 4.42-4.51 (1H, m), 6.80 (1H, s), 7.49-7.52 (2H, m), 7.63-7.70 (4H, m), 7.85-7.86 (1H, m), 7.95-7.98 (2H, m), 9.03 (1H, s), 9.12 (1H, s).

mTOR Kinase Assay (Echo): 0.00576 μM

### EXAMPLE 25k

¹H NMR (400.132 MHz, DMSO-d₆) δ 1.21 (3H, d), 1.24 (6H, s), 1.76-1.79 (2H, m), 1.94-1.97 (2H, m), 2.48-2.49 (3H, m), 3.18 (1H, td), 3.39 (2H, d), 3.47 (1H, td), 3.62 (1H, dd), 3.76 (1H, d), 3.97 (1H, dd), 4.12-4.19 (1H, m), 4.41-4.48 (1H, m), 4.95 (1H, t), 5.98 (1H, s), 6.76 (1H, s), 7.34-7.39 (2H, m), 7.84-7.89 (3H, m), 8.72 (1H, s).

mTOR Kinase Assay (Echo): 0.00292 μM

### EXAMPLE 25l

¹H NMR (400.132 MHz, DMSO-d₆) δ 1.21 (3H, d), 1.60 (2H, quintet), 1.76-1.80 (2H, m), 1.94-1.97 (2H, m), 2.48-2.49 (3H, m), 3.14-3.21 (3H, m), 3.44-3.50 (3H, m), 3.62 (1H, dd), 3.76 (1H, d), 3.97 (1H, dd), 4.12-4.19 (1H, m), 4.42-4.49 (2H, m), 6.18 (1H, t), 6.77 (1H, s), 7.39-7.43 (2H, m), 7.83-7.84 (1H, m), 7.86-7.90 (2H, m), 8.69 (1H, s).

mTOR Kinase Assay (Echo): 0.000956 μM

### EXAMPLE 25m

¹H NMR (400.132 MHz, DMSO-d₆) δ 1.21 (3H, d), 1.77-1.80 (2H, m), 1.94-1.98 (2H, m), 2.48-2.49 (3H, m), 3.15-3.22 (1H, m), 3.48 (1H, td), 3.62 (1H, dd), 3.75-3.79 (4H, m), 3.97 (1H, dd), 4.13-4.20 (1H, m), 4.42-4.49 (1H, m), 6.79 (1H, s), 7.38-7.39 (1H, m), 7.44-7.49 (2H, m), 7.77 (1H, s), 7.84-7.85 (1H, m), 7.90-7.94 (2H, m), 8.38 (1H, s), 8.83 (1H, s).

mTOR Kinase Assay (Echo): 0.00025 μM

The preparation of phenyl N-[4-[4-[(3S)-3-methylmorpholin-4-yl]-6-[1-[(4-methyl-1,3-thiazol-2-yl)sulfonyl]cyclopropyl]pyrimidin-2-yl]phenyl]carbamate is described below:

### Phenyl N-[4-[4-[(3S)-3-methylmorpholin-4-yl]-6-[1-[(4-methyl-1,3-thiazol-2-yl)sulfonyl]cyclopropyl]pyrimidin-2-yl]phenyl]carbamate

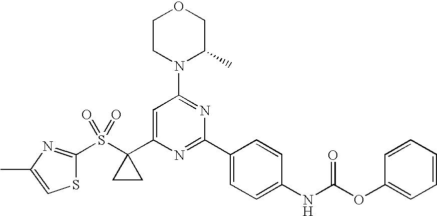

A solution of 4-[4-[(3S)-3-methylmorpholin-4-yl]-6-[1-[(4-methyl-1,3-thiazol-2-yl)sulfonyl]cyclopropyl]pyrimidin-2-yl]aniline (1.56 g, 3.31 mmol) in 1,4-dioxane (18 mL) was treated with sodium bicarbonate (0.445 g, 5.29 mmol). Phenyl chloroformate (0.5 mL, 3.99 mmol) was then added dropwise and the resulting suspension was stirred at RT, under nitrogen for 150 minutes. The reaction mixture was evaporated to dryness and the residue partitioned between DCM (100 mL) and water (50 mL). The organic layer washed with brine, dried (MgSO₄), filtered and evaporated to an amber gum/foam which was triturated under isohexane/diethyl ether (˜1:1 v/v, ˜100 mL) with sonication and resultant solid collected by suction filtration and dried, under vacuum, at 50° C., to give the desired material as a beige solid (1.72 g).

NMR Spectrum: ¹H NMR (400.132 MHz, DMSO-d₆) δ 1.15 (3H, d), 1.71-1.75 (2H, m), 1.87-1.91 (2H, m), 2.40-2.41 (3H, m), 3.12 (1H, td), 3.40 (1H, td), 3.55 (1H, dd), 3.69 (1H, d), 3.90 (1H, dd), 4.07-4.14 (1H, m), 4.35-4.44 (1H, m), 6.75 (1H, s), 7.16-7.23 (3H, m), 7.35-7.40 (2H, m), 7.47-7.50 (2H, m), 7.76-7.77 (1H, m), 7.90-7.93 (2H, m), 10.32 (1H, s).

LCMS Spectrum: m/z (ESI+) (M+H)+=592.1; HPLC tR=2.87 min.

### 4-[4-[(3S)-3-Methylmorpholin-4-yl]-6-[1-[(4-methyl-1,3-thiazol-2-yl)sulfonyl]cyclopropyl]pyrimidin-2-yl]aniline

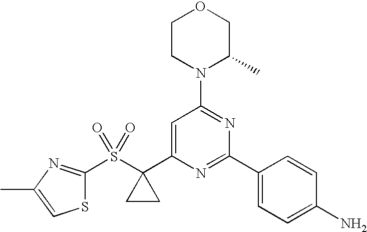

A mixture of 2-chloro-4-[(3S)-3-methylmorpholin-4-yl]-6-[1-[(4-methyl-1,3-thiazol-2-yl)sulfonyl]cyclopropyl]pyrimidine (1.9 g, 4.58 mmol), 4-(4,4,5,5-tetramethyl-1,3,2-dioxaborolan-2-yl)aniline (1.36 g, 6.21 mmol) and 2M aqueous sodium carbonate solution (5.72 mL, 11.45 mmol) in a mixture of ethanol (5.50 mL), DME (11 mL), water (5.50 mL) and DMF (0.7 mL) was purged with nitrogen for 10 minutes before addition of bis(triphenylphosphine)palladium(II) chloride (0.161 g, 0.23 mmol). The reaction mixture was then heated to 85° C. and stirred for 3 hours. The reaction mixture was cooled and partitioned between ethyl acetate (150 mL) and water (250 mL), the organic layer separated and aqueous re-extracted with ethyl acetate (100 mL). The combined organics were washed with brine, dried (MgSO₄), filtered and evaporated to dryness to afford the crude product, which was purified by flash silica chromatography, elution gradient 25 to 75% ethyl acetate in isohexane. The isolated material was further purified by trituration with hot isohexane and diethyl ether to give the desired material as a beige solid (1.6 g).

NMR Spectrum: ¹H NMR (400.132 MHz, DMSO-d₆) δ 1.19 (3H, d), 1.73-1.77 (2H, m), 1.92-1.95 (2H, m), 2.48-2.49 (3H, m), 3.15 (1H, td), 3.46 (1H, td), 3.61 (1H, dd), 3.75 (1H, d), 3.96 (1H, dd), 4.08-4.16 (1H, m), 4.37-4.45 (1H, m), 5.52 (2H, s), 6.50-6.54 (2H, m), 6.67 (1H, s), 7.70-7.74 (2H, m), 7.82-7.84 (1H, m).

LCMS Spectrum: m/z (ESI+) (M+H)+=472.1; HPLC tR=2.24 min.

### 2-Chloro-4-[(3S)-3-methylmorpholin-4-yl]-6-[1-[(4-methyl-1,3-thiazol-2-yl)sulfonyl]cyclopropyl]pyrimidine

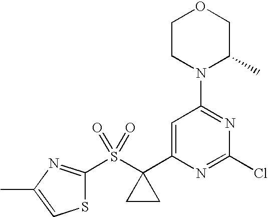

A solution of 2-chloro-4-[(3S)-3-methylmorpholin-4-yl]-6-[(4-methyl-1,3-thiazol-2-yl)sulfonylmethyl]pyrimidine (3.29 g, 8.46 mmol) in toluene (45 mL) was treated with 1,2-dibromoethane (1.4 mL, 16.25 mmol). Tetrabutylammonium bromide (0.273 g, 0.85 mmol) was then added followed by a solution of sodium hydroxide (3.4 g, 85.01 mmol) in water (3.4 mL). The resulting mixture was heated to 65° C., under an atmosphere of nitrogen, for 1 hour then at 75° C. for 2.5 hours. The mixture was allowed to cool and partitioned between ethyl acetate (60 mL) and water (30 mL). The organic layer was separated, washed with brine, dried (MgSO₄), filtered and evaporated to a brown gum. The crude product was purified by flash silica chromatography, elution gradient 25 to 75% ethyl acetate in isohexane, to give the desired material as a yellow gum (2.76 g).

NMR Spectrum: ¹H NMR (400.132 MHz, DMSO-d₆) δ 1.12 (3H, d), 1.63-1.66 (2H, m), 1.81-1.85 (2H, m), 2.39-2.41 (3H, m), 3.11 (1H, td), 3.32-3.38 (1H, m), 3.49 (1H, dd), 3.65 (1H, d), 3.82-3.92 (2H, m), 4.16-4.27 (1H, m), 6.83 (1H, s), 7.81-7.84 (1H, m).

LCMS Spectrum: m/z (ESI+) (M+H)+=415.10; HPLC tR=2.12 min.

### 2-Chloro-4-[(3S)-3-methylmorpholin-4-yl]-6-[(4-methyl-1,3-thiazol-2-yl)sulfonylmethyl]pyrimidine

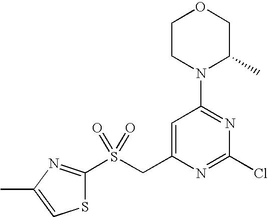

A solution of 2,4-dichloro-6-[(4-methyl-1,3-thiazol-2-yl)sulfonylmethyl]pyrimidine (8.35 g, 25.76 mmol) in DCM (100 mL) was cooled to 4° C. Triethylamine (4.3 mL, 30.85 mmol) was then added and mixture stirred for 5 minutes before dropwise addition, over 10 minutes, of a solution of (S)-3-methylmorpholine (2.9 g, 28.67 mmol) in DCM (25 mL). The reaction mixture was then stirred in cooling bath for 45 minutes then at RT overnight. Water (200 mL) was added to reaction mixture and stirred for 10 minutes before the organic layer was separated and aqueous layer extracted with DCM (50 mL). The combined organic layers were washed with brine, dried (MgSO₄), filtered and evaporated to afford the crude product, which was purified by flash silica chromatography, eluting with 50% ethyl acetate in isohexane, to give the desired material as a yellow gum (5.80 g).

NMR Spectrum: ¹H NMR (400.132 MHz, DMSO-d₆) δ 1.18 (3H, d), 2.49-2.50 (3H, m), 3.18 (1H, td), 3.43 (1H, td), 3.58 (1H, dd), 3.72 (1H, d), 3.88-3.96 (2H, m), 4.15-4.29 (1H, m), 4.82 (2H, s), 6.80 (1H, s), 7.89-7.90 (1H, m).

LCMS Spectrum: m/z (ESI+) (M+H)+=389.2; HPLC tR=1.87 min.

### 2,4-Dichloro-6-[(4-methyl-1,3-thiazol-2-yl)sulfonylmethyl]pyrimidine

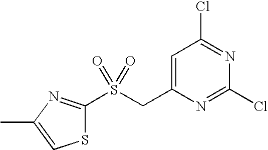

A solution of 2,4-dichloro-6-[(4-methyl-1,3-thiazol-2-yl)sulfanylmethyl]pyrimidine (7.39 g, 25.29 mmol) in DCM (130 ml) was cooled to 4° C. and treated portionwise, over 15 minutes with 3-chloroperoxybenzoic acid (13.60 g, 60.70 mmol). The resulting suspension was stirred in cooling bath for 15 minutes then at RT for 24 hours. A saturated aqueous solution of sodium bicarbonate (200 mL) was added to the reaction mixture and stirred for 30 minutes. The organic layer was separated, washed with brine, dried (MgSO₄), filtered and evaporated to give the desired material as an oil which solidified on standing (8.40 g). The material was used without further purification.

NMR Spectrum: ¹H NMR (400.132 MHz, DMSO-d₆) δ 2.47-2.49 (3H, m), 5.19 (2H, s), 7.84 (1H, s), 7.93-7.95 (1H, m).

LCMS Spectrum: m/z (ESI−) (M−H)−=322.0; HPLC tR=1.53 min.

### 2,4-Dichloro-6-[(4-methyl-1,3-thiazol-2-yl)sulfanylmethyl]pyrimidine

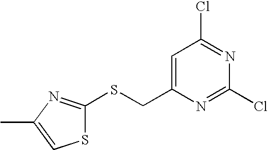

A suspension of 6-[(4-methyl-1,3-thiazol-2-yl)sulfanylmethyl]-1H-pyrimidine-2,4-dione (14.4 g, 56.40 mmol) in phosphorus oxychloride (60 mL, 643.70 mmol) was warmed to 100° C. and stirred for 6 hours. The reaction mixture cooled before evaporating to a brown oil and partitioning between DCM (100 mL) and water (100 mL). With stirring solid sodium hydrogen carbonate was then added carefully to adjust the mixture to pH8, additional aliquots of water (100 mL) and DCM (100 mL) were added during this time. Additional DCM (100 mL) and water (100 mL) were added and the organic layer separated and the aqueous layer re-extracted with DCM (2×100 mL). The combined organic extracts were washed with brine, dried (MgSO₄) and evaporated to dryness to give the desired material as a tan solid (15.76 g). The material was used without further purification.

NMR Spectrum: ¹H NMR (400.132 MHz, DMSO-d₆) δ 2.30-2.32 (3H, m), 4.55 (2H, s), 7.22-7.24 (1H, m), 7.84 (1H, s)

LCMS Spectrum: m/z (ESI+) (M+H)+=292.1; HPLC tR=2.36 min.

### 6-[(4-Methyl-1,3-thiazol-2-yl)sulfanylmethyl]-1H-pyrimidine-2,4-dione

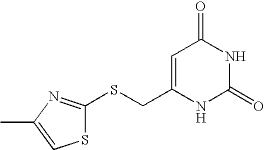

To a solution of 4-methylthiazole-2-thiol (10 g, 76.21 mmol) in DMF (150 mL) at RT was added DBU (14 mL, 93.80 mmol) dropwise over 5 minutes. The resulting solution was stirred at RT for 30 minutes. 6-(Chloromethyl)-1H-pyrimidine-2,4-dione (10 g, 62.28 mmol) was then added portionwise over a period of 20 minutes under nitrogen. The resulting solution was stirred at RT for 19 hours then the reaction mixture evaporated to dryness and the residue partitioned between DCM (150 mL) and water (150 mL). The solid precipitate was collected by filtration to give the desired material as a cream solid (11.1 g). Additional desired material (4.3 g) was obtained after the filtrate was acidified with 2M hydrochloric acid and the resultant precipitate collected by filtration.

NMR Spectrum: ¹H NMR (400.132 MHz, DMSO-d₆) δ 2.34 (3H, s), 4.08 (2H, s), 5.43 (1H, s), 7.27 (1H, s), 10.98 (2H, s)

LCMS Spectrum: m/z (ESI+) (M+H)+=256.2; HPLC tR=0.58 min.

### EXAMPLE 26

### 1-[4-[4-(1-Cyclohexylsulfonylcyclopropyl)-6-[(3S)-3-methylmorpholin-4-yl]pyrimidin-2-yl]phenyl]-3-cyclopropylurea

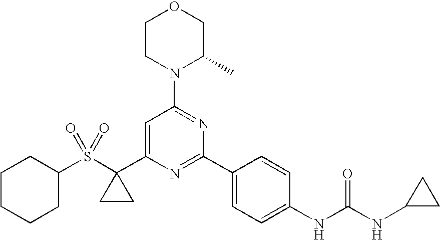

To cyclopropylamine (56 mg, 0.98 mmol) was added a solution of phenyl N-[4-[4-(1-cyclohexylsulfonylcyclopropyl)-6-[(3S)-3-methylmorpholin-4-yl]pyrimidin-2-yl]phenyl]carbamate (79.5 mg, 0.14 mmol) in DMF (2 mL). Triethylamine (0.067 mL, 0.48 mmol) was then added and the resultant mixture was heated to 50° C. and stirred for 2 hours.

The reaction mixture was cooled and purified by preparative HPLC, using decreasingly polar mixtures of water (containing 1% TFA) and acetonitrile as eluants, followed by purification by preparative HPLC, using decreasingly polar mixtures of water (containing 1% ammonia) in acetonitrile as eluants, to give the desired material as a white solid (28 mg).

NMR Spectrum: ¹H NMR (400.132 MHz, DMSO-d₆) δ 0.40-0.44 (2H, m), 0.63-0.67 (2H, m), 1.13-1.29 (6H, m), 1.37-1.66 (7H, m), 1.82-1.88 (2H, m), 2.26-2.34 (2H, m), 2.53-2.59 (1H, m), 3.16-3.25 (1H, m), 3.44-3.53 (2H, m), 3.63 (1H, dd), 3.76 (1H, d), 3.97 (1H, dd), 4.17-4.26 (1H, m), 4.49-4.59 (1H, m), 6.47 (1H, d), 6.76 (1H, s), 7.50-7.54 (2H, m), 8.19-8.23 (2H, m), 8.58 (1H, s).

LCMS Spectrum: m/z (ESI+) (M+H)+=540; HPLC tR=2.57 min.

mTOR Kinase Assay (Echo): 0.00605 μM

The compounds below were prepared in an analogous fashion from phenyl N-[4-[4-(1-cyclohexylsulfonylcyclopropyl)-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 26a | 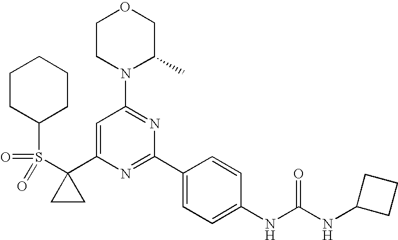 | 3-cyclobutyl-1-[4-[4-(1-cyclohexylsulfonylcyclopropyl)-6-[(3S)-3-methylmorpholin-4-yl]pyrimidin-2-yl]phenyl]urea | 554 | 2.81 |
| 26b | 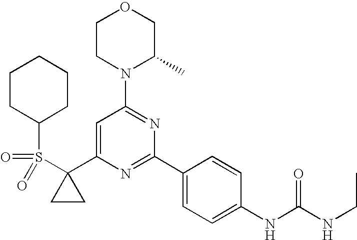 | 3-[4-[4-(1-cyclohexylsulfonylcyclopropyl)-6-[(3S)-3-methylmorpholin-4-yl]pyrimidin-2-yl]phenyl]-1-ethylurea | 528 | 2.56 |
| 26c | 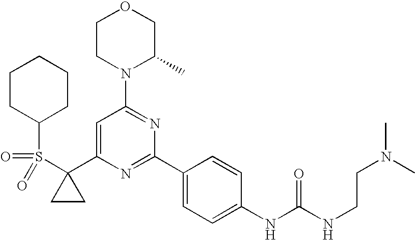 | 1-[4-[4-(1-cyclohexylsulfonylcyclopropyl)-6-[(3S)-3-methylmorpholin-4-yl]pyrimidin-2-yl]phenyl]-3-(2-dimethylaminoethyl)urea | 571 | 2.53 |
| 26d | 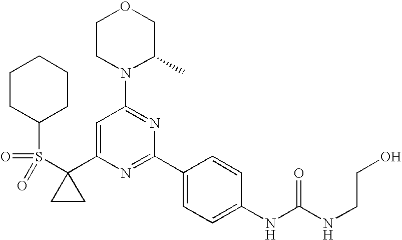 | 1-[4-[4-(1-cyclohexylsulfonylcyclopropyl)-6-[(3S)-3-methylmorpholin-4-yl]pyrimidin-2-yl]phenyl]-3-(2-hydroxyethyl)urea | 544 | 2.15 |
| 26e | 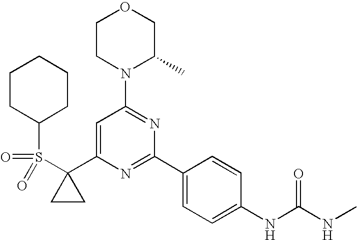 | 1-[4-[4-(1-cyclohexylsulfonylcyclopropyl)-6-[(3S)-3-methylmorpholin-4-yl]pyrimidin-2-yl]phenyl]-3-methylurea | 514 | 2.39 |
| 26f | 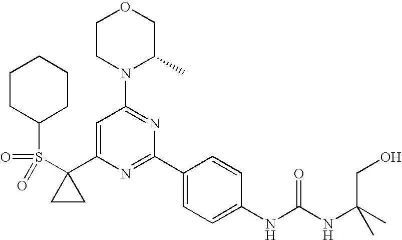 | 1-[4-[4-(1-cyclohexylsulfonylcyclopropyl)-6-[(3S)-3-methylmorpholin-4-yl]pyrimidin-2-yl]phenyl]-3-(1-hydroxy-2-methylpropan-2-yl)urea | 572 | 2.27 |
| 26g | 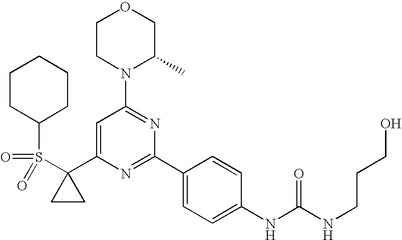 | 1-[4-[4-(1-cyclohexylsulfonylcyclopropyl)-6-[(3S)-3-methylmorpholin-4-yl]pyrimidin-2-yl]phenyl]-3-(3-hydroxypropyl)urea | 558 | 2.21 |
| 26h | 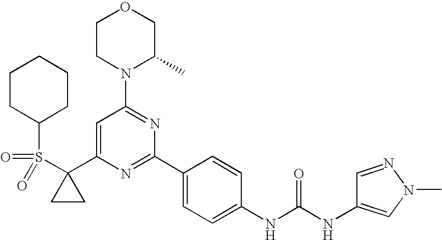 | 1-[4-[4-(1-cyclohexylsulfonylcyclopropyl)-6-[(3S)-3-methylmorpholin-4-yl]pyrimidin-2-yl]phenyl]-3-(1-methylpyrazol-4-yl)urea | 580 | 2.40 |

### EXAMPLE 26a

¹H NMR (400.132 MHz, DMSO-d₆) δ 1.13-1.28 (6H, m), 1.37-1.69 (9H, m), 1.81-1.92 (4H, m), 2.17-2.25 (2H, m), 2.26-2.34 (2H, m), 3.20 (1H, td), 3.44-3.52 (2H, m), 3.63 (1H, dd), 3.76 (1H, d), 3.97 (1H, dd), 4.09-4.25 (2H, m), 4.50-4.58 (1H, m), 6.49 (1H, d), 6.76 (1H, s), 7.47-7.50 (2H, m), 8.19-8.22 (2H, m), 8.60 (1H, s).

mTOR Kinase Assay (Echo): 0.00672 μM

### EXAMPLE 26b

¹H NMR (400.132 MHz, DMSO-d₆) δ 1.07 (3H, t), 1.13-1.29 (6H, m), 1.36-1.66 (7H, m), 1.81-1.89 (2H, m), 2.26-2.35 (2H, m), 3.09-3.16 (2H, m), 3.20 (1H, td), 3.45-3.53 (2H, m), 3.63 (1H, dd), 3.76 (1H, d), 3.97 (1H, dd), 4.17-4.25 (1H, m), 4.50-4.58 (1H, m), 6.20 (1H, t), 6.76 (1H, s), 7.48-7.52 (2H, m), 8.18-8.23 (2H, m), 8.70 (1H, s).

mTOR Kinase Assay (Echo): 0.0039 μM

### EXAMPLE 26c

¹H NMR (400.132 MHz, DMSO-d₆) δ 1.12-1.29 (6H, m), 1.36-1.66 (7H, m), 1.82-1.88 (2H, m), 2.18 (6H, s), 2.27-2.35 (4H, m), 3.16-3.24 (3H, m), 3.44-3.52 (2H, m), 3.63 (1H, dd), 3.76 (1H, d), 3.97 (1H, dd), 4.17-4.25 (1H, m), 4.50-4.58 (1H, m), 6.17 (1H, t), 6.76 (1H, s), 7.47-7.51 (2H, m), 8.19-8.23 (2H, m), 8.90 (1H, s).

mTOR Kinase Assay (Echo): 0.119 μM

### EXAMPLE 26d

¹H NMR (400.132 MHz, DMSO-d₆) δ 1.11-1.28 (6H, m), 1.38-1.65 (7H, m), 1.81-1.88 (2H, m), 2.27-2.34 (2H, m), 3.16-3.24 (3H, m), 3.44-3.52 (4H, m), 3.63 (1H, dd), 3.76 (1H, d), 3.97 (1H, dd), 4.17-4.25 (1H, m), 4.50-4.58 (1H, m), 4.73 (1H, t), 6.27 (1H, t), 6.76 (1H, s), 7.48-7.51 (2H, m), 8.19-8.23 (2H, m), 8.83 (1H, s).

mTOR Kinase Assay (Echo): 0.0012 μM

### EXAMPLE 26e

¹H NMR (400.132 MHz, DMSO-d₆) δ 1.13-1.29 (6H, m), 1.37-1.66 (7H, m), 1.82-1.88 (2H, m), 2.27-2.34 (2H, m), 2.66 (3H, d), 3.20 (1H, td), 3.45-3.53 (2H, m), 3.63 (1H, dd), 3.76 (1H, d), 3.97 (1H, dd), 4.17-4.25 (1H, m), 4.50-4.59 (1H, m), 6.09 (1H, q), 6.76 (1H, s), 7.49-7.53 (2H, m), 8.19-8.22 (2H, m), 8.77 (1H, s).

mTOR Kinase Assay (Echo): 0.00395 μM

### EXAMPLE 26f

¹H NMR (400.132 MHz, DMSO-d₆) δ 1.12-1.29 (12H, m), 1.37-1.67 (7H, m), 1.82-1.88 (2H, m), 2.27-2.34 (2H, m), 3.20 (1H, td), 3.39 (2H, d), 3.45-3.53 (2H, m), 3.63 (1H, dd), 3.76 (1H, d), 3.97 (1H, dd), 4.17-4.25 (1H, m), 4.49-4.59 (1H, m), 4.95 (1H, t), 6.01 (1H, s), 6.75 (1H, s), 7.44-7.47 (2H, m), 8.18-8.22 (2H, m), 8.77 (1H, s).

mTOR Kinase Assay (Echo): 0.00457 μM

### EXAMPLE 26g

¹H NMR (400.132 MHz, DMSO-d₆) δ 1.14-1.29 (6H, m), 1.37-1.66 (9H, m), 1.81-1.88 (2H, m), 2.27-2.34 (2H, m), 3.14-3.24 (3H, m), 3.44-3.52 (4H, m), 3.63 (1H, dd), 3.76 (1H, d), 3.97 (1H, dd), 4.18-4.24 (1H, m), 4.47 (1H, t), 4.50-4.58 (1H, m), 6.23 (1H, t), 6.76 (1H, s), 7.48-7.52 (2H, m), 8.19-8.22 (2H, m), 8.74 (1H, s).

mTOR Kinase Assay (Echo): 0.00746 μM

### EXAMPLE 26h

¹H NMR (400.132 MHz, DMSO-d₆) δ 1.13-1.30 (6H, m), 1.37-1.67 (7H, m), 1.82-1.89 (2H, m), 2.28-2.34 (2H, m), 3.21 (1H, td), 3.45-3.53 (2H, m), 3.64 (1H, dd), 3.74-3.79 (4H, m), 3.98 (1H, dd), 4.18-4.26 (1H, m), 4.51-4.59 (1H, m), 6.77 (1H, s), 7.39 (1H, d), 7.54-7.57 (2H, m), 7.77 (1H, s), 8.23-8.26 (2H, m), 8.42 (1H, s), 8.87 (1H, s).

mTOR Kinase Assay (Echo): 0.004 μM

The preparation of phenyl N-[4-[4-(1-cyclohexylsulfonylcyclopropyl)-6-[(3S)-3-methylmorpholin-4-yl]pyrimidin-2-yl]phenyl]carbamate is described below:

### Phenyl N-[4-[4-(1-cyclohexylsulfonylcyclopropyl)-6-[(3S)-3-methylmorpholin-4-yl]pyrimidin-2-yl]phenyl]carbamate

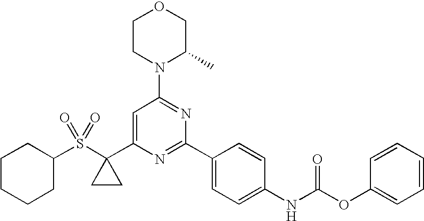

A suspension of 4-[4-(1-cyclohexylsulfonylcyclopropyl)-6-[(3S)-3-methylmorpholin-4-yl]pyrimidin-2-yl]aniline (615 mg, 1.35 mmol) in 1,4-dioxane (7 mL) was treated with sodium bicarbonate (182 mg, 2.17 mmol). Phenyl chloroformate (0.20 mL, 1.59 mmol) was then added dropwise and resultant mixture left to stir under nitrogen at RT overnight (˜16 hours). The reaction mixture was evaporated to dryness and the residue partitioned between DCM (10 mL) and water (10 mL). The organic layer was separated and evaporated to an amber gum which was azeotroped with diethyl ether to give the desired material as a beige solid (726 mg).

NMR Spectrum: ¹H NMR (400.132 MHz, DMSO-d₆) δ 1.13-1.29 (6H, m), 1.37-1.48 (2H, m), 1.50-1.65 (5H, m), 1.81-1.87 (2H, m), 2.26-2.34 (2H, m), 3.22 (1H, td), 3.44-3.52 (2H, m), 3.64 (1H, dd), 3.77 (1H, d), 3.98 (1H, dd), 4.20-4.29 (1H, m), 4.52-4.60 (1H, m), 6.83 (1H, s), 7.23-7.30 (3H, m), 7.42-7.47 (2H, m), 7.63-7.67 (2H, m), 8.28-8.32 (2H, m), 10.45 (1H, s).

LCMS Spectrum: m/z (ESI+) (M+H)+=577.1; HPLC tR=3.12 min.

### 4-[4-(1-Cyclohexylsulfonylcyclopropyl)-6-[(3S)-3-methylmorpholin-4-yl]pyrimidin-2-yl]aniline

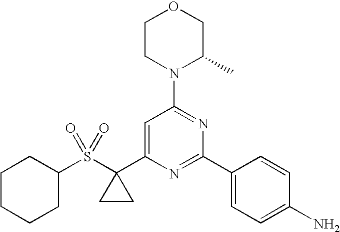

A mixture of 2-chloro-4-(1-cyclohexylsulfonylcyclopropyl)-6-[(3S)-3-methylmorpholin-4-yl]pyrimidine (1.78 g, 4.45 mmol) and 4-(4,4,5,5-tetramethyl-1,3,2-dioxaborolan-2-yl)aniline (1.474 g, 6.73 mmol) in a mixture of DMF (8 mL), ethanol (8 mL), DME (8 mL) and water (20 mL) was treated with 2M aqueous sodium carbonate solution (11 mL, 22.00 mmol). The resulting mixture was purged with nitrogen for 10 minutes before addition of bis(triphenylphosphine)palladium(II) chloride (0.156 g, 0.22 mmol). The mixture was heated to 85° C. and stirred, under nitrogen for 4 hours before being cooled and partitioned between ethyl acetate (100 mL) and water (100 mL). The biphasic mixture was filtered and the organic layer was separated. The aqueous layer was re-extracted with ethyl acetate (2×50 mL) and the combined organics were washed with brine, dried (MgSO₄) and evaporated to afford the crude product, which was purified by flash silica chromatography, elution gradient 25 to 75% ethyl acetate in isohexane, to give the desired material as a beige solid (0.627 g).

NMR Spectrum: ¹H NMR (400.132 MHz, DMSO-d₆) δ 1.07-1.21 (6H, m), 1.31-1.49 (6H, m), 1.53-1.58 (1H, m), 1.74-1.82 (2H, m), 2.19-2.26 (2H, m), 3.07-3.14 (1H, m), 3.37-3.47 (2H, m), 3.55 (1H, dd), 3.68 (1H, d), 3.89 (1H, dd), 4.07-4.14 (1H, m), 4.40-4.48 (1H, m), 5.50 (2H, s), 6.54 (2H, d), 6.58 (1H, s), 7.98 (2H, d).

LCMS Spectrum: m/z (ESI+) (M+H)+=457.3; HPLC tR=2.55 min.

### 2-Chloro-4-(1-cyclohexylsulfonylcyclopropyl)-6-[(3S)-3-methylmorpholin-4-yl]pyrimidine

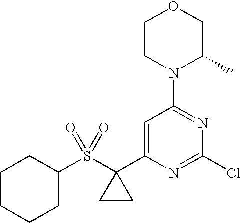

A solution of 2-chloro-4-(cyclohexylsulfonylmethyl)-6-[(3S)-3-methylmorpholin-4-yl]pyrimidine (2.8 g, 7.49 mmol) in toluene (40 mL) was treated with 1,2-dibromoethane (1.3 mL, 15.09 mmol). Tetrabutylammonium bromide (0.241 g, 0.75 mmol) was then added followed by a solution of sodium hydroxide (3.00 g, 74.89 mmol) in water (3 mL). The resulting mixture was heated to 64° C. and stirred for 90 minutes. The reaction mixture was cooled before addition of ethyl acetate (50 mL) and water (20 mL). The mixture was stirred for 5 minutes then the organic layer separated, washed with brine (30 mL) and evaporated to afford the crude product, which was purified by flash silica chromatography, elution gradient 25 to 75% ethyl acetate in isohexane, to give the desired compound as a pale yellow oil which crystallised on standing (1.785 g).

NMR Spectrum: ¹H NMR (400.132 MHz, DMSO-d₆) δ 1.10-1.41 (8H, m), 1.48-1.55 (4H, m), 1.60-1.66 (1H, m), 1.77-1.84 (2H, m), 2.14-2.20 (2H, m), 3.16-3.24 (1H, m), 3.32-3.47 (2H, m), 3.58 (1H, dd), 3.72 (1H, d), 3.93 (1H, dd), 3.99-4.07 (1H, m), 4.32-4.43 (1H, m), 6.93 (1H, s).

LCMS Spectrum: m/z (ESI+) (M+H)+=400.3; HPLC tR=2.56 min.

### 2-Chloro-4-(cyclohexylsulfonylmethyl)-6-[(3S)-3-methylmorpholin-4-yl]pyrimidine

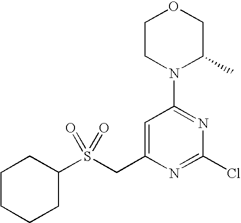

To a solution of 2,4-dichloro-6-(cyclohexylsulfonylmethyl)pyrimidine (1.7 g, 5.50 mmol) in DCM (25 ml), cooled with a water/ice bath, was added triethylamine (0.85 mL, 6.10 mmol). The resulting solution was treated, dropwise over 5 minutes, with a solution of (S)-3-methylmorpholine (0.658 g, 6.51 mmol) in DCM (5 mL). The mixture was stirred in cooling bath for 30 minutes then at RT for 3 hours. Water (25 mL) was added to the reaction mixture and stirred for 15 minutes. The organic layer was separated, dried (MgSO₄), filtered and evaporated to give the crude product, which was purified by flash silica chromatography, elution gradient 25 to 75% ethyl acetate in isohexane, to give the desired material as a yellow oil which crystallised on standing (1.3 g).

NMR Spectrum: ¹H NMR (400.132 MHz, DMSO-d₆) δ 1.16-1.45 (8H, m), 1.63-1.69 (1H, m), 1.81-1.87 (2H, m), 2.11-2.17 (2H, m), 3.16-3.25 (2H, m), 3.45 (1H, td), 3.60 (1H, dd), 3.73 (1H, d), 3.92-4.05 (2H, m), 4.26-4.34 (1H, m), 4.40 (2H, s), 6.90 (1H, s).

LCMS Spectrum: m/z (ESI+) (M+H)+=374.3; HPLC tR=2.22 min.

### 2,4-Dichloro-6-(cyclohexylsulfonylmethyl)pyrimidine

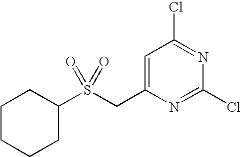

To a solution of 2,4-dichloro-6-(cyclohexylsulfanylmethyl)pyrimidine (4.23 g, 15.26 mmol) in DCM (90 mL), cooled in a water/ice bath, was added 3-chloroperoxybenzoic acid (8.55 g, 38.15 mmol) over a period of 30 minutes under nitrogen, so as to control temperature below 10° C. The resulting suspension was stirred at RT for 3 hours. Saturated aqueous sodium hydrogen carbonate solution (120 mL) was then carefully added portionwise and reaction mixture stirred for 30 minutes before separating the organic layer, drying (MgSO₄) and evaporating to give the desired material as an off white solid (4.90 g). The material was used without further purification.

NMR Spectrum: ¹H NMR (400.132 MHz, DMSO-d₆) δ 1.13-1.46 (5H, m), 1.62-1.68 (1H, m), 1.81-1.87 (2H, m), 2.10-2.16 (2H, m), 3.22 (1H, tt), 4.74 (2H, s), 7.85 (1H, s).

LCMS Spectrum: m/z (ESI−) (M−H)−=307.2; HPLC tR=2.22 min.

### 2,4-Dichloro-6-(cyclohexylsulfanylmethyl)pyrimidine

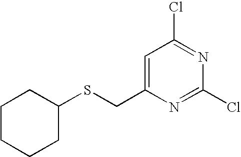

A suspension of 6-(cyclohexylsulfanylmethyl)-1H-pyrimidine-2,4-dione (6.4 g, 26.63 mmol) in phosphorus oxychloride (25 mL, 268.2 mmol) was warmed to 100° C., over a period of 15 minutes. The resulting dark orange solution was stirred at 100° C. for 7 hours before being cooled and evaporated to a brown oil. The oil was partitioned between DCM (150 mL) and water (150 mL). With stirring solid sodium hydrogen carbonate was then added carefully to adjust the mixture to pH8, aliquots of water (100 mL) and DCM (50 mL) were added during addition. The organic layer was separated, the aqueous layer was re-extracted with more DCM (2×75 mL) and the combined organic layers washed with brine (200 mL), dried (MgSO₄) and evaporated to afford crude product. The crude product was purified by flash silica chromatography, eluting with 15% ethyl acetate in isohexane, to give the desired material as an orange liquid (4.24 g).

NMR Spectrum: ¹H NMR (400.132 MHz, DMSO-d₆) δ 1.20-1.31 (5H, m), 1.51-1.57 (1H, m), 1.64-1.72 (2H, m), 1.86-1.92 (2H, m), 2.71-2.77 (1H, m), 3.85 (2H, s), 7.82 (1H, s).

LCMS Spectrum: m/z (ESI−) (M−H)−=275.2 & 277.2 HPLC tR=3.01 min.

### 6-(Cyclohexylsulfanylmethyl)-1H-pyrimidine-2,4-dione

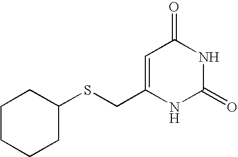

A solution of cyclohexanethiol (10 mL, 81.74 mmol) in DMF (150 mL) at RT was treated with DBU (14 mL, 93.80 mmol). The resulting solution was stirred at RT for 20 minutes then 6-(chloromethyl)-1H-pyrimidine-2,4-dione (10 g, 62.28 mmol) added portionwise over a period of 30 minutes, under nitrogen, so as to maintain the internal temperature below 35° C. The resulting solution was stirred at RT overnight. The reaction mixture was evaporated to dryness and the residue was partitioned between DCM (100 mL) and water (150 mL). On mixing a precipitate formed, this was removed by filtration to give the desired material as a white solid (6.45 g). Additional desired material (3.62 g) was obtained by separating the filtrate, adjusting the aqueous layer to pH2 and collecting the resultant precipitate by filtration.

NMR Spectrum: ¹H NMR (400.132 MHz, DMSO-d₆) δ 1.16-1.34 (5H, m), 1.51-1.58 (1H, m), 1.63-1.72 (2H, m), 1.87-1.96 (2H, m), 2.65-2.72 (1H, m), 3.41 (2H, s), 5.49 (1H, s), 10.75-10.96 (2H, m).

LCMS Spectrum: m/z (ESI+) (M+H)+=241.3; HPLC tR=0.99 min.

### EXAMPLE 27

### 1-[4-[4-[1-(4-Chlorophenyl)sulfonylcyclopropyl]-6-[(3S)-3-methylmorpholin-4-yl]pyrimidin-2-yl]phenyl]-3-cyclopropylurea

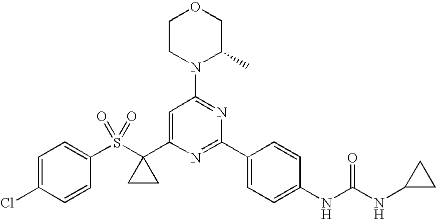

Cyclopropylamine (0.137 mL, 1.98 mmol) was added to phenyl N-[4-[4-[1-(4-chlorophenyl)sulfonylcyclopropyl]-6-[(3S)-3-methylmorpholin-4-yl]pyrimidin-2-yl]phenyl]carbamate (150 mg, 0.25 mmol) in DMF (2 mL). The resulting solution was stirred at 60° C. for 4 hours. The mixture was evaporated to dryness and the residue was partitioned between ethyl acetate (15 mL) and water (15 mL). The organic layer was washed with 1M aqueous citric acid (15 mL) and water (15 mL) and evaporated to dryness. The residue was purified by preparative HPLC, using decreasingly polar mixtures of water (containing 1% ammonia) and acetonitrile as eluents, to afford the desired material as a white solid (62 mg).

NMR Spectrum: ¹H NMR (400.132 MHz, CDCl₃) δ 0.69 (2H, m), 0.87 (2H, m), 1.32 (3H, d), 1.57 (1H, ddd), 1.64 (1H, ddd), 1.98 (2H, m), 2.63 (1H, m), 3.29 (1H, ddd), 3.59 (1H, ddd), 3.74 (1H, dd), 3.83 (1H, d), 4.04 (1H, dd), 4.14 (1H, d), 4.41 (1H, br.d), 4.87 (1H, s), 6.77 (1H, s), 6.93 (1H, s), 7.40 (2H, d), 7.42 (2H, d), 7.68 (2H, d), 7.99 (2H, d).

LCMS Spectrum: m/z (ESI+)(M+H)+=568, 570; HPLC tR=2.33 min.

mTOR Kinase Assay (Echo): 0.00144 μM

The compounds below were prepared in an analogous fashion from phenyl N-[4-[4-[1-(4-chlorophenyl)sulfonylcyclopropyl]-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 27a | 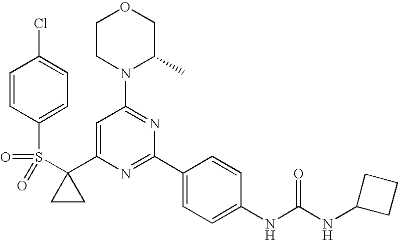 | 1-[4-[4-[1-(4-chlorophenyl)sulfonylcyclopropyl]-6-[(3S)-3-methylmorpholin-4-yl]pyrimidin-2-yl]phenyl]-3-cyclobutylurea | 582,584 | 2.55 |
| 27b | 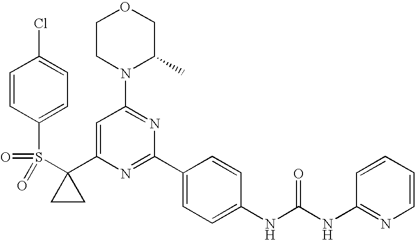 | 1-[4-[4-[1-(4-chlorophenyl)sulfonylcyclopropyl]-6-[(3S)-3-methylmorpholin-4-yl]pyrimidin-2-yl]phenyl]-3-pyridin-2-ylurea | 605,607 | 2.73 |
| 27c | 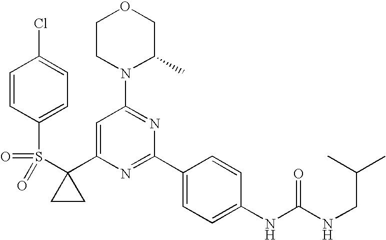 | 1-[4-[4-[1-(4-chlorophenyl)sulfonylcyclopropyl]-6-[(3S)-3-methylmorpholin-4-yl]pyrimidin-2-yl]phenyl]-3-(2-methylpropyl)urea | 584,586 | 2.63 |
| 27d | 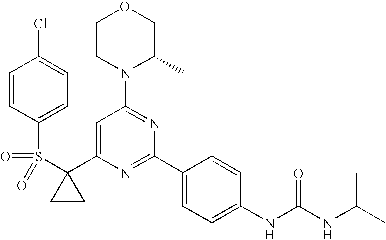 | 1-[4-[4-[1-(4-chlorophenyl)sulfonylcyclopropyl]-6-[(3S)-3-methylmorpholin-4-yl]pyrimidin-2-yl]phenyl]-3-propan-2-ylurea | 570,572 | 2.47 |
| 27e | 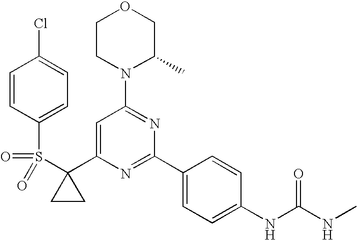 | 1-[4-[4-[1-(4-chlorophenyl)sulfonylcyclopropyl]-6-[(3S)-3-methylmorpholin-4-yl]pyrimidin-2-yl]phenyl]-3-methylurea | 542,544 | 2.17 |
| 27f | 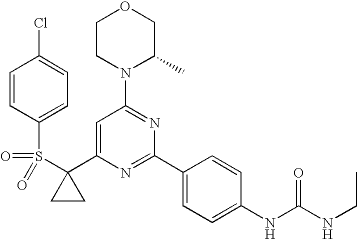 | 3-[4-[4-[1-(4-chlorophenyl)sulfonylcyclopropyl]-6-[(3S)-3-methylmorpholin-4-yl]pyrimidin-2-yl]phenyl]-1-ethylurea | 556,558 | 2.33 |
| 27g | 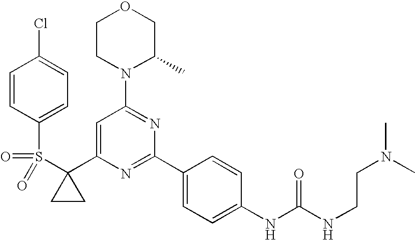 | 1-[4-[4-[1-(4-chlorophenyl)sulfonylcyclopropyl]-6-[(3S)-3-methylmorpholin-4-yl]pyrimidin-2-yl]phenyl]-3-(2-dimethylaminoethyl)urea | 599,601 | 2.23 |
| 27h | 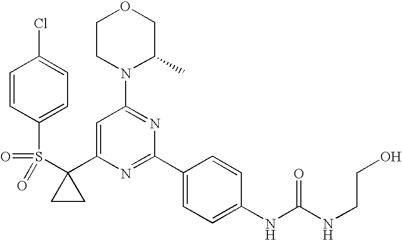 | 1-[4-[4-[1-(4-chlorophenyl)sulfonylcyclopropyl]-6-[(3S)-3-methylmorpholin-4-yl]pyrimidin-2-yl]phenyl]-3-(2-hydroxyethyl)urea | 572,574 | 2.00 |
| 27i | 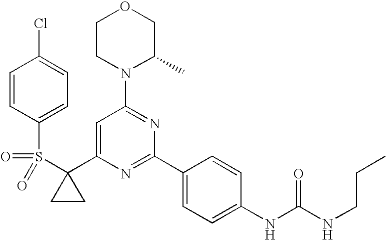 | 3-[4-[4-[1-(4-chlorophenyl)sulfonylcyclopropyl]-6-[(3S)-3-methylmorpholin-4-yl]pyrimidin-2-yl]phenyl]-1-propylurea | 570,572 | 2.48 |
| 27j | 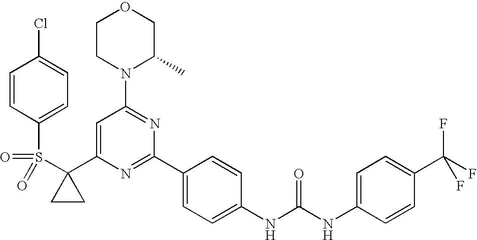 | 1-[4-[4-[1-(4-chlorophenyl)sulfonylcyclopropyl]-6-[(3S)-3-methylmorpholin-4-yl]pyrimidin-2-yl]phenyl]-3-[4-(trifluoromethyl)phenyl]urea | 672,674 | 3.09 |
| 27k | 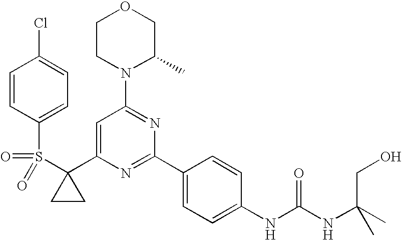 | 1-[4-[4-[1-(4-chlorophenyl)sulfonylcyclopropyl]-6-[(3S)-3-methylmorpholin-4-yl]pyrimidin-2-yl]phenyl]-3-(1-hydroxy-2-methylpropan-2-yl)urea | 600,602 | 2.28 |
| 27l | 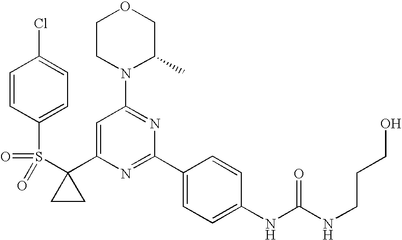 | 1-[4-[4-[1-(4-chlorophenyl)sulfonylcyclopropyl]-6-[(3S)-3-methylmorpholin-4-yl]pyrimidin-2-yl]phenyl]-3-(3-hydroxypropyl)urea | 586,588 | 2.04 |
| 27m | 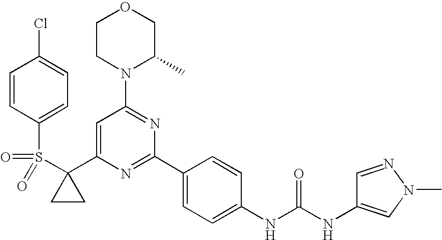 | 1-[4-[4-[1-(4-chlorophenyl)sulfonylcyclopropyl]-6-[(3S)-3-methylmorpholin-4-yl]pyrimidin-2-yl]phenyl]-3-(1-methylpyrazol-4-yl)urea | 608,610 | 2.38 |

### EXAMPLE 27a

¹H NMR (399.902 MHz, CDCl₃) δ 1.31 (3H, d), 1.59 (2H, m), 1.71 (2H, m), 1.84 (2H, m), 1.98 (2H, m), 2.38 (2H, m), 3.29 (1H, ddd), 3.59 (1H, ddd), 3.74 (1H, dd), 3.83 (1H, d), 4.04 (1H, dd), 4.13 (1H, d), 4.30 (1H, tt), 4.41 (1H, br.d), 4.82 (1H, d), 6.26 (1H, s), 6.76 (1H, s), 7.30 (2H, d), 7.40 (2H, d), 7.67 (2H, d), 7.98 (2H, d).

mTOR Kinase Assay (Echo): 0.00388 μM

### EXAMPLE 27b

¹H NMR (399.902 MHz, CDCl₃) δ 1.32 (3H, d), 1.59 (1H, ddd), 1.66 (1H, ddd), 1.99 (2H, m), 3.30 (1H, ddd), 3.61 (1H, ddd), 3.75 (1H, dd), 3.83 (1H, d), 4.05 (1H, dd), 4.15 (1H, d), 4.42 (1H, m), 6.71 (1H, d), 6.78 (1H, s), 6.98 (1H, dd), 7.25 (1H, s), 7.41 (2H, d), 7.60 (2H, d), 7.66 (1H, ddd), 7.69 (2H, d), 8.03 (2H, d), 8.30 (1H, d), 11.92 (1H, s).

mTOR Kinase Assay (Echo): 0.00425 μM

### EXAMPLE 27c

¹H NMR (399.902 MHz, CDCl₃) δ 0.94 (6H, d), 1.31 (3H, d), 1.56 (1H, ddd), 1.63 (1H, ddd), 1.80 (1H, m), 1.98 (2H, m), 3.10 (2H, dd), 3.29 (1H, ddd), 3.59 (1H, ddd), 3.74 (1H, dd), 3.82 (1H, d), 4.04 (1H, dd), 4.13 (1H, d), 4.41 (1H, br.d), 4.81 (1H, t), 6.41 (1H, s), 6.75 (1H, s), 7.30 (2H, d), 7.40 (2H, d), 7.67 (2H, d), 7.98 (2H, d).

mTOR Kinase Assay (Echo): 0.014 μM

### EXAMPLE 27d

¹H NMR (399.902 MHz, CDCl₃) δ 1.19 (6H, d), 1.31 (3H, d), 1.56 (1H, ddd), 1.63 (1H, ddd), 1.98 (2H, m), 3.29 (1H, ddd), 3.59 (1H, ddd), 3.74 (1H, dd), 3.82 (1H, d), 4.03 (2H, m), 4.13 (1H, d), 4.41 (1H, d), 4.54 (1H, br.d), 6.31 (1H, s), 6.75 (1H, s), 7.29 (2H, d), 7.40 (2H, d), 7.67 (2H, d), 7.97 (2H, d).

mTOR Kinase Assay (Echo): 0.00307 μM

### EXAMPLE 27e

¹H NMR (399.902 MHz, CDCl₃) δ 1.32 (3H, d), 1.56 (1H, ddd), 1.62 (1H, ddd), 1.98 (2H, m), 2.87 (3H, d), 3.29 (1H, ddd), 3.59 (1H, ddd), 3.74 (1H, dd), 3.83 (1H, d), 4.04 (1H, dd), 4.13 (1H, d), 4.41 (1H, br.d), 4.65 (1H, q), 6.31 (1H, s), 6.76 (1H, s), 7.30 (2H, d), 7.40 (2H, d), 7.67 (2H, d), 7.98 (2H, d).

mTOR Kinase Assay (Echo): 0.000719 μM

### EXAMPLE 27f

¹H NMR (399.902 MHz, CDCl₃) δ 1.17 (3H, t), 1.31 (3H, d), 1.56 (1H, ddd), 1.62 (1H, ddd), 1.98 (2H, m), 3.28 (3H, ddd), 3.32 (3H, dq), 3.59 (1H, ddd), 3.74 (1H, dd), 3.82 (1H, d), 4.04 (1H, dd), 4.13 (1H, d), 4.41 (1H, br.d), 4.71 (1H, t), 6.38 (1H, s), 6.75 (1H, s), 7.30 (2H, d), 7.40 (2H, d), 7.67 (2H, d), 7.98 (2H, d).

mTOR Kinase Assay (Echo): 0.000959 μM

### EXAMPLE 27g

¹H NMR (399.902 MHz, CDCl₃) δ 1.31 (3H, d), 1.56 (1H, ddd), 1.63 (1H, ddd), 1.97 (2H, m), 2.31 (6H, s), 2.51 (2H, t), 3.28 (3H, ddd), 3.32 (3H, dt), 3.59 (1H, ddd), 3.74 (1H, dd), 3.82 (1H, d), 4.04 (1H, dd), 4.14 (1H, d), 4.41 (1H, br.d), 5.25 (1H, br.t), 6.74 (1H, s), 7.26 (1H, s), 7.35 (2H, d), 7.40 (2H, d), 7.67 (2H, d), 7.95 (2H, d).

mTOR Kinase Assay (Echo): 0.0189 μM

### EXAMPLE 27h

¹H NMR (399.902 MHz, CDCl₃) δ 1.31 (3H, d), 1.56 (1H, ddd), 1.62 (1H, ddd), 1.98 (2H, m), 2.64 (1H, br.s), 3.28 (1H, ddd), 3.43 (2H, dt), 3.59 (1H, ddd), 3.73 (1H, dd), 3.75 (2H, m), 3.82 (1H, d), 4.04 (1H, dd), 4.12 (1H, d), 4.40 (1H, br.d), 5.27 (1H, t), 6.73 (1H, s), 6.81 (1H, s), 7.31 (2H, d), 7.40 (2H, d), 7.67 (2H, d), 7.98 (2H, d).

mTOR Kinase Assay (Echo): 0.0000856 μM

### EXAMPLE 27i

¹H NMR (399.902 MHz, CDCl₃) δ 0.94 (3H, t), 1.31 (3H, d), 1.56 (2H, m), 1.56 (1H, ddd), 1.63 (1H, ddd), 1.98 (2H, m), 3.24 (2H, dt), 3.30 (1H, dd), 3.59 (1H, ddd), 3.74 (1H, dd), 3.82 (1H, d), 4.04 (1H, dd), 4.13 (1H, d), 4.41 (1H, br.d), 4.74 (1H, t), 6.36 (1H, s), 6.75 (1H, s), 7.30 (2H, d), 7.40 (2H, d), 7.67 (2H, d), 7.98 (2H, d).

mTOR Kinase Assay (Echo): 0.00302 μM

### EXAMPLE 27j

¹H NMR (399.902 MHz, CDCl₃) δ 1.31 (3H, d), 1.57 (1H, ddd), 1.63 (1H, ddd), 1.99 (2H, m), 3.29 (1H, ddd), 3.59 (1H, ddd), 3.74 (1H, dd), 3.83 (1H, d), 4.04 (1H, dd), 4.12 (1H, d), 4.40 (1H, br.d), 6.72 (1H, s), 6.77 (1H, s), 6.88 (1H, s), 7.37 (2H, d), 7.41 (2H, d), 7.50 (2H, d), 7.55 (2H, d), 7.69 (2H, d), 8.03 (2H, d).

mTOR Kinase Assay (Echo): 0.00761 μM

### EXAMPLE 27k

¹H NMR (399.902 MHz, CDCl₃) δ 1.31 (3H, d), 1.57 (1H, ddd), 1.63 (1H, ddd), 1.98 (2H, m), 3.29 (1H, ddd), 3.59 (1H, ddd), 3.64 (2H, d), 3.73 (1H, dd), 3.82 (1H, d), 4.04 (1H, dd), 4.13 (1H, d), 4.40 (1H, br.d), 4.45 (1H, t), 4.88 (1H, s), 6.54 (1H, s), 6.75 (1H, s), 7.27 (2H, d), 7.40 (2H, d), 7.67 (2H, d), 7.99 (2H, d).

mTOR Kinase Assay (Echo): 0.00462 μM

### EXAMPLE 27l

¹H NMR (399.902 MHz, CDCl₃) δ 1.31 (3H, d), 1.56 (1H, ddd), 1.62 (1H, ddd), 1.71 (2H, tt), 1.98 (2H, m), 2.94 (1H, br.s), 3.28 (1H, ddd), 3.44 (2H, dt), 3.59 (1H, ddd), 3.72 (1H, br.s), 3.73 (1H, dd), 3.82 (1H, d), 4.04 (1H, dd), 4.12 (1H, d), 4.40 (1H, br.d), 5.13 (1H, t), 6.62 (1H, s), 6.74 (1H, s), 7.30 (2H, d), 7.40 (2H, d), 7.67 (2H, d), 7.98 (2H, d).

mTOR Kinase Assay (Echo): 0.00112 μM

### EXAMPLE 27m

¹H NMR (399.902 MHz, CDCl₃) δ 1.31 (3H, d), 1.57 (1H, ddd), 1.63 (1H, ddd), 1.98 (2H, m), 3.29 (1H, ddd), 3.59 (1H, ddd), 3.74 (1H, dd), 3.83 (1H, d), 3.90 (3H, s), 4.04 (1H, dd), 4.13 (1H, d), 4.40 (1H, br.d), 6.12 (1H, s), 6.63 (1H, s), 6.76 (1H, s), 7.35 (2H, d), 7.40 (2H, d), 7.41 (1H, s), 7.60 (1H, s), 7.67 (2H, d), 7.99 (2H, d).

mTOR Kinase Assay (Echo): 0.00144 μM

The preparation of phenyl N-[4-[4-[1-(4-chlorophenyl)sulfonylcyclopropyl]-6-[(3S)-3-methylmorpholin-4-yl]pyrimidin-2-yl]phenyl]carbamate is described below.

### Phenyl N-[4-[4-[1-(4-chlorophenyl)sulfonylcyclopropyl]-6-[(3S)-3-methylmorpholin-4-yl]pyrimidin-2-yl]phenyl]carbamate

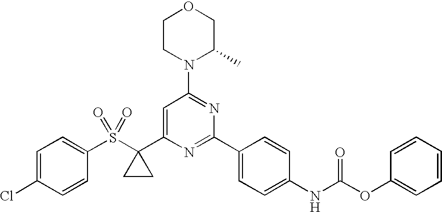

Phenyl chloroformate (0.829 mL, 6.60 mmol) was added dropwise to 4-[4-[1-(4-chlorophenyl)sulfonylcyclopropyl]-6-[(3S)-3-methylmorpholin-4-yl]pyrimidin-2-yl]aniline (3.20 g, 6.60 mmol) and sodium hydrogen carbonate (0.554 g, 6.60 mmol) in 1,4-dioxane (150 mL) at RT. The resulting suspension was stirred at RT for 2 hours. The reaction mixture was diluted with ethyl acetate (400 mL) and washed with water (2×400 mL) and saturated brine (200 mL). The organic layer was dried (MgSO₄), filtered and evaporated. The crude product was purified by flash silica chromatography, elution gradient 30 to 50% ethyl acetate in isohexane, to afford the desired material as a white dry film (3.78 g).

NMR Spectrum: ¹H NMR (399.902 MHz, CDCl₃) δ 1.32 (3H, d), 1.57 (1H, ddd), 1.64 (1H, ddd), 1.99 (2H, m), 3.30 (1H, ddd), 3.60 (1H, ddd), 3.74 (1H, dd), 3.83 (1H, d), 4.05 (1H, dd), 4.15 (2H, br.d), 4.42 (1H, br.d), 6.78 (1H, s), 7.04 (1H, s), 7.20 (2H, d), 7.25 (2H, dd), 7.40 (2H, d), 7.40 (2H, dd), 7.45 (2H, d), 7.68 (2H, d), 8.02 (2H, d).

LCMS Spectrum: m/z (ESI+)(M+H)+=605, 607; HPLC tR=3.15 min.

### 4-[4-[1-(4-Chlorophenyl)sulfonylcyclopropyl]-6-[(3S)-3-methylmorpholin-4-yl]pyrimidin-2-yl]aniline

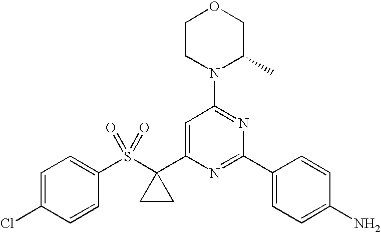

Sodium carbonate (13.45 mL, 26.89 mmol) was added to 4-(4,4,5,5-tetramethyl-1,3,2-dioxaborolan-2-yl)aniline (1.637 g, 7.47 mmol), 2-chloro-4-[1-(4-chlorophenyl)sulfonylcyclopropyl]-6-[(3S)-3-methylmorpholin-4-yl]pyrimidine (3.20 g, 7.47 mmol) in a mixture of DME (20 mL), DMF (20 mL), ethanol (20 mL) and water (40 mL) at RT under nitrogen. The mixture was degassed and purged with nitrogen before bis(triphenylphosphine)palladium(II) chloride (0.262 g, 0.37 mmol) was added and the mixture stirred at 80° C. under nitrogen for 90 minutes. The reaction mixture was concentrated and diluted with ethyl acetate (250 mL), and washed sequentially with water (2×200 mL) and saturated brine (150 mL). The organic layer was dried (MgSO₄), filtered and evaporated. The crude product was purified by flash silica chromatography, elution gradient 30 to 45% ethyl acetate in isohexane, to afford the desired material as a beige dry film (3.32 g).

NMR Spectrum: ¹H NMR (399.902 MHz, CDCl₃) δ 1.30 (3H, d), 1.56 (1H, ddd), 1.62 (1H, ddd), 1.96 (2H, m), 3.27 (1H, ddd), 3.59 (1H, ddd), 3.73 (1H, dd), 3.81 (1H, d), 3.86 (2H, s), 4.03 (1H, dd), 4.13 (1H, br.d), 4.41 (1H, br.d), 6.63 (2H, d), 6.70 (1H, s), 7.40 (2H, d), 7.67 (2H, d), 7.85 (2H, d).

LCMS Spectrum: m/z (ESI+)(M+H)+=485, 487; HPLC tR=2.70 min.

### 2-Chloro-4-[1-(4-chlorophenyl)sulfonylcyclopropyl]-6-[(3S)-3-methylmorpholin-4-yl]pyrimidine

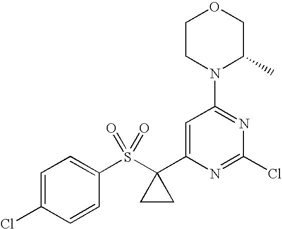

10N Sodium hydroxide solution (7.46 mL, 74.57 mmol) was added to 2-chloro-4-[(4-chlorophenyl)sulfonylmethyl]-6-[(3S)-3-methylmorpholin-4-yl]pyrimidine (3.00 g, 7.46 mmol), 1,2-dibromoethane (1.285 mL, 14.91 mmol) and tetrabutylammonium bromide (0.240 g, 0.75 mmol) in toluene (50 mL). The resulting solution was stirred at 60° C. for 1 hour. The reaction mixture was concentrated, diluted with ethyl acetate (300 mL), washed with water (2×300 mL) and saturated brine (200 mL). The organic layer was dried (MgSO₄), filtered and evaporated. The crude product was purified by flash silica chromatography, elution gradient 30 to 50% ethyl acetate in isohexane, to afford the desired material as a white dry film (2.85 g).

NMR Spectrum: ¹H NMR (399.902 MHz, CDCl₃) δ 1.31 (3H, d), 1.54 (1H, ddd), 1.60 (1H, ddd), 1.95 (2H, m), 3.27 (1H, ddd), 3.54 (1H, ddd), 3.69 (1H, dd), 3.79 (1H, d), 4.01 (2H, m), 4.27 (1H, br.s), 6.87 (1H, s), 7.46 (2H, d), 7.64 (2H, d).

LCMS Spectrum: m/z (ESI+)(M+H)+=428, 430; HPLC tR=2.51 min.

The preparation of 2-chloro-4-[(4-chlorophenyl)sulfonylmethyl]-6-[(3S)-3-methylmorpholin-4-yl]pyrimidine was described earlier.

### EXAMPLE 28

### 3-Cyclopropyl-1-[4-[4-[(3S)-3-methylmorpholin-4-yl]-6-(1-pyridin-2-ylsulfonylcyclopropyl)pyrimidin-2-yl]phenyl]urea

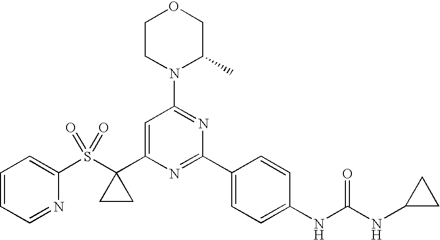

Cyclopropylamine (0.76 mmol) was added in one portion to phenyl N-[4-[4-[(3S)-3-methylmorpholin-4-yl]-6-(1-pyridin-2-ylsulfonylcyclopropyl)pyrimidin-2-yl]phenyl]carbamate (104 mg, 0.19 mmol), in DMF (2 mL) at RT under nitrogen. The resulting solution was stirred at RT for 60 minutes. The reaction mixture was purified by preparative HPLC, using decreasingly polar mixtures of water (containing 1% ammonia) and acetonitrile as eluents, to afford the desired material as a white solid (49 mg).

NMR Spectrum: ¹H NMR (399.902 DMSO-d₆) δ 0.33 (2H, s), 0.54-0.60 (2H, m), 1.06-1.10 (3H, m), 1.62-1.70 (2H, m), 1.92 (2H, s), 3.01-3.11 (1H, m), 3.37 (1H, t), 3.52 (1H, d), 3.66 (1H, d), 3.87 (1H, d), 4.00-4.11 (1H, m), 4.32 (1H, s), 6.33 (1H, s), 6.59 (1H, s), 7.29 (2H, d), 7.61-7.69 (3H, m), 7.89-7.93 (1H, m), 7.98-8.03 (1H, m), 8.41 (1H, s), 8.75 (1H, s);

LCMS Spectrum: m/z (ESI+)(M+H)+=535.4; HPLC tR=1.91 min.

mTOR Kinase Assay (Echo): 0.000816 μM

The compounds below were prepared in an analogous fashion from phenyl N-[4-[4-[(3S)-3-methylmorpholin-4-yl]-6-(1-pyridin-2-ylsulfonylcyclopropyl)pyrimidin-2-yl]phenyl]carbamate using the appropriate amine.

| | | | LCMS | Retention |
|---|---|---|---|---|
| Example | Structure | NAME | MH+ | time (min) |
| 28a | 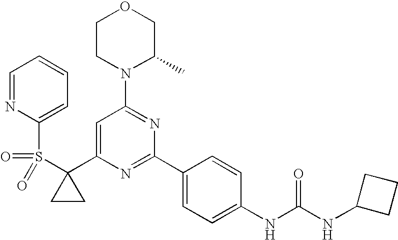 | 3-cyclobutyl-1-[4-[4-[(3S)-3-methylmorpholin-4-yl]-6-(1-pyridin-2-ylsulfonylcyclopropyl)pyrimidin-2-yl]phenyl]urea | 549.4 | 2.13 |
| 28b | 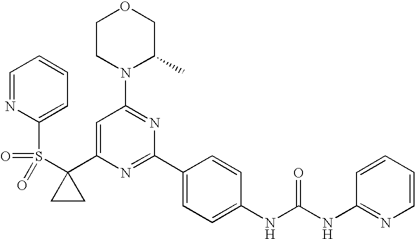 | 1-[4-[4-[(3S)-3-methylmorpholin-4-yl]-6-(1-pyridin-2-ylsulfonylcyclopropyl)pyrimidin-2-yl]phenyl]-3-pyridin-2-ylurea | 572.4 | 2.32 |
| 28c | 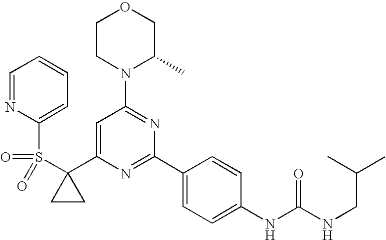 | 1-[4-[4-[(3S)-3-methylmorpholin-4-yl]-6-(1-pyridin-2-ylsulfonylcyclopropyl)pyrimidin-2-yl]phenyl]-3-(2-methylpropyl)urea | 551.4 | 2.22 |
| 28d | 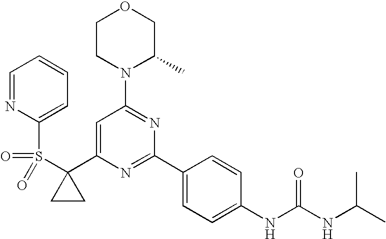 | 1-[4-[4-[(3S)-3-methylmorpholin-4-yl]-6-(1-pyridin-2-ylsulfonylcyclopropyl)pyrimidin-2-yl]phenyl]-3-propan-2-ylurea | 537.9 | 2.06 |
| 28e | 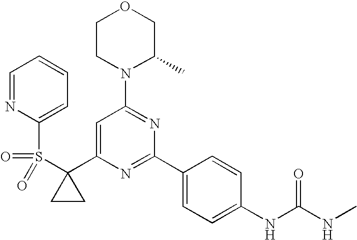 | 3-methyl-1-[4-[4-[(3S)-3-methylmorpholin-4-yl]-6-(1-pyridin-2-ylsulfonylcyclopropyl)pyrimidin-2-yl]phenyl]urea | 509.4 | 1.73 |
| 28f | 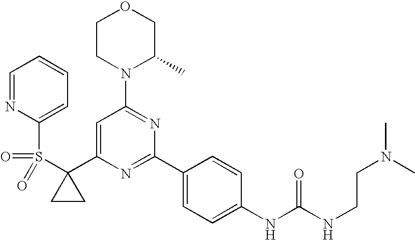 | 3-(2-dimethylaminoethyl)-1-[4-[4-[(3S)-3-methylmorpholin-4-yl]-6-(1-pyridin-2-ylsulfonylcyclopropyl)pyrimidin-2-yl]phenyl]urea | 564.4 | 1.81 |
| 28g | 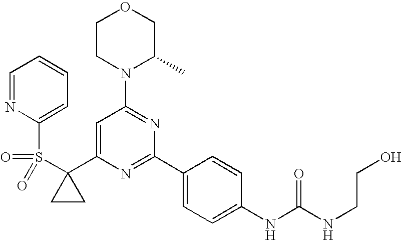 | 3-(2-hydroxyethyl)-1-[4-[4-[(3S)-3-methylmorpholin-4-yl]-6-(1-pyridin-2-ylsulfonylcyclopropyl)pyrimidin-2-yl]phenyl]urea | 539.4 | 1.6 |
| 28h | 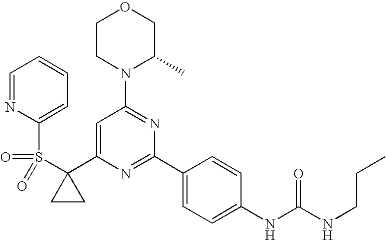 | 3-[4-[4-[(3S)-3-methylmorpholin-4-yl]-6-(1-pyridin-2-ylsulfonylcyclopropyl)pyrimidin-2-yl]phenyl]-1-propylurea | 537.4 | 2.07 |
| 28i | 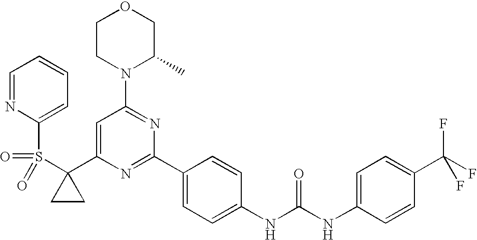 | 1-[4-[4-[(3S)-3-methylmorpholin-4-yl]-6-(1-pyridin-2-ylsulfonylcyclopropyl)pyrimidin-2-yl]phenyl]-3-[4-(trifluoromethyl)phenyl]urea | 639.4 | 2.82 |
| 28j | 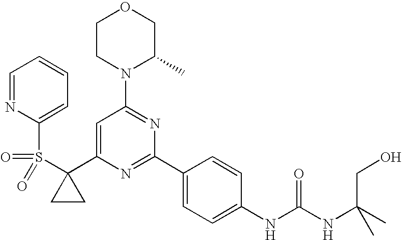 | 3-(1-hydroxy-2-methylpropan-2-yl)-1-[4-[4-[(3S)-3-methylmorpholin-4-yl]-6-(1-pyridin-2-ylsulfonylcyclopropyl)pyrimidin-2-yl]phenyl]urea | 567.4 | 1.87 |
| 28k | 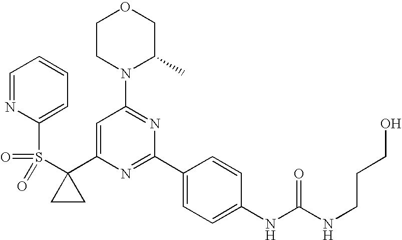 | 3-(3-hydroxypropyl)-1-[4-[4-[(3S)-3-methymorpholin-4-yl]-6-(1-pyridin-2-ylsulfonylcyclopropyl)pyrimidin-2-yl]phenyl]urea | 553.4 | 1.63 |
| 28l | 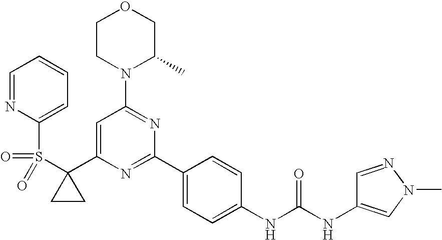 | 1-[4-[4-[(3S)-3-methylmorpholin-4-yl]-6-(1-pyridin-2-ylsulfonylcyclopropyl)pyrimidin-2-yl]phenyl]-3-(1-methylpyrazol-4-yl)urea | 575.4 | 1.8 |

### EXAMPLE 28a

¹H NMR (399.902 DMSO-d₆) δ 1.16 (d, 3H), 1.57-1.68 (m, 2H), 1.70-1.78 (m, 2H), 1.80-1.92 (m, 2H), 1.96-2.04 (m, 2H), 2.17-2.26 (m, 2H), 3.10-3.18 (m, 1H), 3.41-3.49 (m, 1H), 3.57-3.63 (m, 1H), 3.75 (d, 1H), 3.93-3.98 (m, 1H), 4.09-4.19 (m, 2H), 4.40 (s, 1H), 6.44 (d, 1H), 6.67 (s, 1H), 7.34 (d, 2H), 7.72 (d, 2H), 7.73-7.77 (m, 1H), 7.99 (d, 1H), 8.06-8.11 (m, 1H), 8.52 (s, 1H), 8.81-8.84 (m, 1H).

mTOR Kinase Assay (Echo): 0.00253 μM

### EXAMPLE 28b

¹H NMR (399.902 DMSO-d₆) δ 1.18 (d, 3H), 1.71-1.82 (m, 2H), 1.98-2.05 (m, 2H), 3.11-3.21 (m, 1H), 3.42-3.51 (m, 1H), 3.59-3.64 (m, 1H), 3.76 (d, 1H), 3.94-4.00 (m, 1H), 4.15 (d, 1H), 4.42 (s, 1H), 6.71 (s, 1H), 7.02-7.07 (m, 1H), 7.50 (d, 2H), 7.56-7.60 (m, 1H), 7.75-7.80 (m, 2H), 7.81 (d, 2H), 7.99-8.02 (m, 1H), 8.08-8.13 (m, 1H), 8.29-8.32 (m, 1H), 8.83-8.85 (m, 1H), 9.42-9.44 (m, 1H), 10.53 (s, 1H).

mTOR Kinase Assay (Echo): 0.00149 μM

### EXAMPLE 28c

¹H NMR (399.902 DMSO-d₆) δ 0.79 (d, 6H), 1.07 (d, 3H), 1.57-1.68 (m, 3H), 1.88-1.92 (m, 2H), 2.84 (t, 2H), 3.00-3.09 (m, 1H), 3.31-3.40 (m, 1H), 3.48-3.53 (m, 1H), 3.65 (d, 1H), 3.84-3.89 (m, 1H), 4.00-4.07 (m, 1H), 4.30 (s, 1H), 6.13 (t, 1H), 6.57 (s, 1H), 7.24-7.28 (m, 2H), 7.62 (d, 2H), 7.64-7.67 (m, 1H), 7.88-7.91 (m, 1H), 7.97-8.02 (m, 1H), 8.51 (s, 1H), 8.72-8.74 (m, 1H).

mTOR Kinase Assay (Echo): 0.00847 μM

### EXAMPLE 28d

¹H NMR (399.902 DMSO-d₆) δ 1.11 (d, 6H), 1.16 (d, 3H), 1.70-1.79 (m, 2H), 1.96-2.04 (m, 2H), 3.10-3.18 (m, 1H), 3.41-3.49 (m, 1H), 3.57-3.63 (m, 1H), 3.72-3.83 (m, 2H), 3.93-3.99 (m, 1H), 4.13 (d, 1H), 4.40 (s, 1H), 6.04 (d, 1H), 6.67 (s, 1H), 7.34 (d, 2H), 7.72 (d, 2H), 7.74-7.77 (m, 1H), 7.98-8.01 (m, 1H), 8.06-8.12 (m, 1H), 8.49 (s, 1H), 8.82-8.84 (m, 1H).

mTOR Kinase Assay (Echo): 0.00237 μM

### EXAMPLE 28e

¹H NMR (399.902 DMSO-d₆) δ 1.16 (d, 3H), 1.70-1.80 (m, 2H), 1.96-2.03 (m, 2H), 2.66 (d, 3H), 3.10-3.18 (m, 1H), 3.41-3.49 (m, 1H), 3.58-3.63 (m, 1H), 3.75 (d, 1H), 3.93-3.98 (m, 1H), 4.13 (d, 1H), 4.41 (s, 1H), 6.05 (q, 1H), 6.67 (s, 1H), 7.37 (d, 2H), 7.72 (d, 2H), 7.73-7.77 (m, 1H), 7.99 (d, 1H), 8.07-8.12 (m, 1H), 8.70 (s, 1H), 8.82-8.84 (m, 1H).

mTOR Kinase Assay (Echo): 0.000434 μM

### EXAMPLE 28f

¹H NMR (399.902 DMSO-d₆) δ 1.17 (d, 3H), 1.69-1.80 (m, 2H), 1.97-2.03 (m, 2H), 2.19 (s, 6H), 2.34 (t, 3H), 3.10-3.22 (m, 3H), 3.41-3.49 (m, 1H), 3.58-3.63 (m, 1H), 3.75 (d, 1H), 3.94-3.98 (m, 1H), 4.12 (d, 1H), 4.40 (s, 1H), 6.15 (t, 1H), 6.67 (s, 1H), 7.35 (d, 2H), 7.72 (d, 2H), 7.74-7.78 (m, 1H), 8.00 (d, 1H), 8.09 (t, 1H), 8.81-8.87 (m, 2H).

mTOR Kinase Assay (Echo): 0.0674 μM

### EXAMPLE 28g

¹H NMR (399.902 DMSO-d₆) δ 1.14-1.21 (m, 3H), 1.70-1.79 (m, 2H), 1.97-2.04 (m, 2H), 3.10-3.24 (m, 3H), 3.41-3.51 (m, 3H), 3.57-3.65 (m, 1H), 3.75 (d, 1H), 3.93-3.99 (m, 1H), 4.13 (d, 1H), 4.41 (s, 1H), 4.73 (t, 1H), 6.23 (t, 1H), 6.67 (s, 1H), 7.35 (d, 2H), 7.70-7.77 (m, 3H), 7.99 (d, 1H), 8.09 (t, 1H), 8.76 (s, 1H), 8.83 (d, 1H).

mTOR Kinase Assay (Echo): 0.0118 μM

### EXAMPLE 28h

¹H NMR (399.902 DMSO-d₆) δ 0.87-0.94 (m, 3H), 1.15-1.21 (m, 3H), 1.42-1.52 (m, 2H), 2.01 (s, 2H), 3.03-3.20 (m, 3H), 3.47 (t, 1H), 3.62 (d, 1H), 3.76 (d, 1H), 3.97 (d, 1H), 4.15 (d, 1H), 4.42 (s, 1H), 6.20 (s, 1H), 6.69 (s, 1H), 7.37 (d, 2H), 7.70-7.79 (m, 3H), 7.98-8.03 (m, 1H), 8.07-8.14 (m, 1H), 8.63 (s, 1H), 8.84 (s, 1H).

mTOR Kinase Assay (Echo): 0.00093 μM

### EXAMPLE 28i

¹H NMR (399.902 DMSO-d₆) δ 1.18 (d, 4H), 1.72-1.79 (m, 2H), 2.00-2.02 (m, 2H), 3 (d, 1H), 3.11-3.20 (m, 1H), 3.43-3.50 (m, 2H), 3.59-3.64 (m, 1H), 3.75 (d, 1H), 3.94-3.99 (m, 2H), 4.14 (d, 1H), 4.42 (s, 1H), 6.70 (s, 1H), 7.45 (d, 2H), 7.67 (q, 4H), 7.74-7.82 (m, 3H), 8.08-8.13 (m, 1H), 8.82-8.85 (m, 1H), 9.01 (s, 1H), 9.11 (s, 1H).

mTOR Kinase Assay (Echo): 0.00153 μM

### EXAMPLE 28j

¹H NMR (399.902 DMSO-d₆) δ 1.16 (d, 3H), 1.24 (s, 6H), 1.68-1.80 (m, 2H), 1.96-2.04 (m, 2H), 3.09-3.18 (m, 1H), 3.39 (d, 2H), 3.41-3.49 (m, 1H), 3.58-3.63 (m, 1H), 3.75 (d, 1H), 3.93-3.98 (m, 1H), 4.13 (d, 1H), 4.40 (s, 1H), 4.96 (t, 1H), 5.98 (s, 1H), 6.67 (s, 1H), 7.32 (d, 2H), 7.71 (d, 2H), 7.74-7.78 (m, 1H), 7.99 (d, 1H), 8.09 (t, 1H), 8.70 (s, 1H), 8.82-8.85 (m, 1H).

mTOR Kinase Assay (Echo): 0.00557 μM

### EXAMPLE 28k

¹H NMR (399.902 DMSO-d₆) δ 1.17 (d, 3H), 1.56-1.64 (m, 2H), 1.69-1.80 (m, 2H), 1.96-2.04 (m, 2H), 3.10-3.20 (m, 3H), 3.41-3.51 (m, 3H), 3.58-3.63 (m, 1H), 3.74 (d, 1H), 3.93-3.98 (m, 1H), 4.13 (d, 1H), 4.40 (s, 1H), 4.48 (t, 1H), 6.19 (t, 1H), 6.67 (s, 1H), 7.36 (d, 2H), 7.72 (d, 2H), 7.73-7.77 (m, 1H), 7.99 (d, 1H), 8.09 (t, 1H), 8.67 (s, 1H), 8.82-8.84 (m, 1H).

mTOR Kinase Assay (Echo): 0.000954 μM

### EXAMPLE 28l

¹H NMR (399.902 DMSO-d₆) δ 1.17 (d, 3H), 1.69-1.81 (m, 2H), 1.96-2.05 (m, 2H), 3.10-3.19 (m, 1H), 3.42-3.50 (m, 1H), 3.58-3.64 (m, 1H), 3.75 (d, 1H), 3.79 (s, 3H), 3.93-3.99 (m, 1H), 4.14 (d, 1H), 4.41 (s, 1H), 6.68 (s, 1H), 7.38-7.44 (m, 3H), 7.73-7.78 (m, 4H), 8.00 (d, 1H), 8.07-8.12 (m, 1H), 8.37 (s, 1H), 8.80 (s, 1H), 8.82-8.85 (m, 1H).

mTOR Kinase Assay (Echo): 0.000442 μM

The preparation of phenyl N-[4-[4-[(3S)-3-methylmorpholin-4-yl]-6-(1-pyridin-2-ylsulfonylcyclopropyl)pyrimidin-2-yl]phenyl]carbamate is described below.

### Phenyl N-[4-[4-[(3S)-3-methylmorpholin-4-yl]-6-(1-pyridin-2-ylsulfonylcyclopropyl)pyrimidin-2-yl]phenyl]carbamate

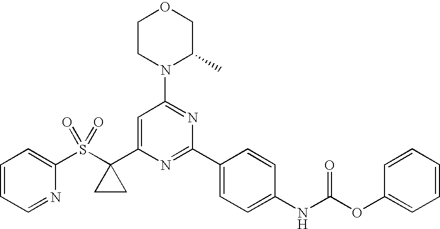

Phenyl chloroformate (0.693 g, 4.43 mmol) was added dropwise to 4-[4-[(3S)-3-methylmorpholin-4-yl]-6-(1-pyridin-2-ylsulfonylcyclopropyl)pyrimidin-2-yl]aniline (2.0 g, 4.43 mmol) and sodium hydrogen carbonate (0.744 g, 8.86 mmol) in dioxane (40 mL) at RT. The resulting slurry was stirred at RT for 1 hour. The mixture was partitioned between ethyl acetate and water. The organic solution was dried (MgSO₄) and concentrated under reduced pressure. The residue was purified by chromatography on silica, eluting with 0%-20% ethyl acetate in DCM, to afford the desired material as a yellow gum (2.07 g).

NMR spectrum: ¹H NMR (399.902 CDCl₃) δ 1.23 (5H, d), 1.61-1.71 (2H, m), 2.06-2.15 (2H, m), 3.17-3.24 (1H, m), 3.47-3.54 (1H, m), 3.75 (1H, d), 4.05-4.11 (2H, m), 4.35 (1H, s), 6.82 (1H, s), 6.95 (1H, s), 7.13 (2H, m), 7.16-7.21 (1H, m), 7.31-7.37 (4H, m), 7.38-7.43 (1H, m), 7.73-7.77 (1H, m), 7.86 (1H, d), 7.93-7.97 (2H, m)

LCMS Spectrum: m/z (ESI+)(M+H)+=572.6; HPLC tR=2.81 min.

### 4-[4-[(3S)-3-Methylmorpholin-4-yl]-6-(1-pyridin-2-ylsulfonylcyclopropyl)pyrimidin-2-yl]aniline

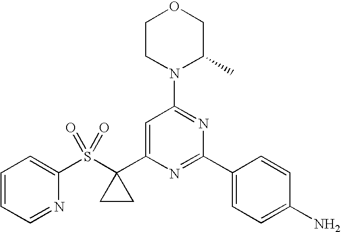

Sodium carbonate (20.51 mL, 41.03 mmol) was added to 4-(4,4,5,5-tetramethyl-1,3,2-dioxaborolan-2-yl)aniline (2.498 g, 11.4 mmol) and 2-chloro-4-[(3S)-3-methylmorpholin-4-yl]-6-(1-pyridin-2-ylsulfonylcyclopropyl)pyrimidine (4.5 g, 11.40 mmol) in a mixture of DME (20 mL), ethanol (20 mL), DMF (20 mL) and water (40 mL). The mixture was purged with nitrogen then bis(triphenylphosphine)palladium(II) chloride (0.4 g, 0.57 mmol) was added and the resulting suspension was stirred at 80° C. for 90 minutes. The reaction mixture was concentrated, diluted with ethyl acetate (150 mL), and washed sequentially with water (2×150 mL) and saturated brine (100 mL). The organic layer was dried (MgSO₄), filtered and evaporated to afford crude product. The crude product was purified by flash silica chromatography, elution gradient 30 to 45% ethyl acetate in DCM, to afford the desired material as a beige solid (3.64 g).

NMR spectrum: ¹H NMR (399.902 CDCl₃) δ 1.20 (3H, d), 1.60-1.72 (2H, m), 2.04-2.12 (2H, m), 3.14-3.22 (1H, m), 3.46-3.53 (1H, m), 3.71-3.78 (3H, m), 3.92-3.96 (1H, m), 4.02-4.08 (1H, m), 4.33 (1H, s), 6.53 (2H, d), 6.76 (1H, s), 7.37-7.41 (1H, m), 7.72-7.77 (1H, m), 7.78-7.86 (3H, m), 8.64-8.66 (1H, m)

LCMS Spectrum: m/z (ESI+)(M+H)+=452.6; HPLC tR=1.40 min.

### 2-Chloro-4-[(3S)-3-methylmorpholin-4-yl]-6-(1-pyridin-2-ylsulfonylcyclopropyl)pyrimidine

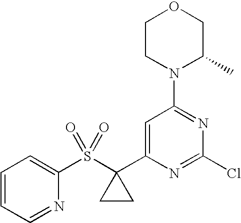

1,2-Dibromoethane (5.61 mL, 65.08 mmol) was added to 2-chloro-4-[(3S)-3-methylmorpholin-4-yl]-6-(pyridin-2-ylsulfonylmethyl)pyrimidine (6 g, 16.27 mmol), 10N sodium hydroxide solution (32.5 mL, 325.35 mmol) and tetrabutylammonium bromide (0.524 g, 1.63 mmol) in toluene (400 mL). The resulting solution was stirred at 60° C. for 3 hours. The reaction mixture was concentrated and diluted with ethyl acetate (150 mL), then washed with water (2×100 mL) and saturated brine (50 mL). The organic layer was dried (MgSO₄), filtered and evaporated to afford crude product. The crude product was purified by flash silica chromatography, elution gradient 5 to 20% ethyl acetate in DCM, to afford the desired material as a white dry film (3.02 g).

NMR spectrum: ¹H NMR (399.902 CDCl₃) δ 1.22 (3H, d), 1.56-1.67 (2H, m), 1.97-2.07 (2H, m), 3.14-3.22 (1H, m), 3.41-3.49 (1H, m), 3.58-3.62 (1H, m), 3.70 (1H, d), 3.89-3.99 (2H, m), 4.21 (1H, s), 7.02 (1H, s), 7.44-7.48 (1H, m), 7.81-7.89 (2H, m), 8.62-8.64 (1H, m)

LCMS Spectrum: m/z (ESI+)(M+H)+=395.4; HPLC tR=1.98 min.

### 2-Chloro-4-[(3S)-3-methylmorpholin-4-yl]-6-(pyridin-2-ylsulfonylmethyl)pyrimidine

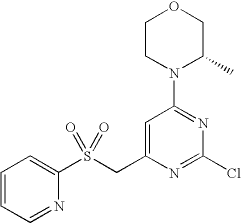

A 35% aqueous solution of hydrogen peroxide (8.26 mL, 93.53 mmol) was added dropwise to a stirred solution of 2-chloro-4-[(3S)-3-methylmorpholin-4-yl]-6-(pyridin-2-ylsulfanylmethyl)pyrimidine (10.5 g, 31.17 mmol), sodium tungstate dihydrate (0.206 g, 0.62 mmol) and 2N Sulfuric acid (0.6 mL) in dioxane (300 mL) and then the solution warmed to 55° C. The solution was stirred at 55° C. for 4 hours. Further hydrogen peroxide (8.26 mL) was added and the mixture stirred at 50° C. for 18 hours. 3-Chloroperoxybenzoic acid (5.38 g, 31.17 mmol) was added and the mixture stirred at RT for 2 hours. The solution was diluted with water (500 mL) and cooled to 20° C. A 10% solution of sodium metabisulfite was added to destroy any remaining peroxide and the solution was extracted with ethyl acetate. The organic layer was dried (MgSO₄) and filtered. The crude product was purified by flash silica chromatography, elution gradient 0 to 50% ethyl acetate in DCM, to give the desired material as a yellow gum (10.5 g,).

NMR Spectrum: ¹H NMR (399.902 MHz, CDCl₃) δ 1.24 (d, 3H), 3.20 (m, 1H), 3.46 (m, 1H), 3.61 (d, 1H), 3.71 (d, 1H), 3.90-3.98 (m, 2H), 4.21 (s, 1H), 4.51 (s, 2H), 6.50 (s, 1H), 7.51-7.53 (m, 1H), 7.86-7.95 (m, 2H), 8.72-8.74 (m, 1H)

LCMS Spectrum: m/z (ESI+)(M+H)+=369.4; HPLC tR=1.73 min.

### 2-Chloro-4-[(3S)-3-methylmorpholin-4-yl]-6-(pyridin-2-ylsulfanylmethyl)pyrimidine

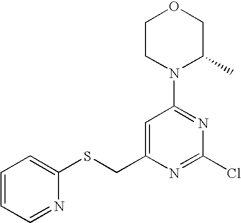

DIPEA (8.77 ml, 50.71 mmol) was added to 2-mercaptopyridine (3.80 g, 34.22 mmol), in DMF (300 mL) at RT in an atmosphere of nitrogen. The resulting solution was stirred at RT for 15 minutes. 2-Chloro-4-(iodomethyl)-6-[(3S)-3-methylmorpholin-4-yl]pyrimidine (11 g, 31.11 mmol) was added portionwise over 5 minutes and the mixture stirred at RT for 3 hours. The reaction mixture was evaporated to dryness and redissolved in DCM (200 mL) and washed sequentially with saturated sodium hydrogen carbonate solution (100 mL) and saturated brine (50 mL). The organic layer was dried (MgSO₄), filtered and evaporated to afford crude product. The crude product was purified by flash silica chromatography, elution gradient 0 to 20% ethyl acetate in DCM, to give the desired material as a tan oil (10.50 g). NMR shows the presence of 0.6 eq. of 3-chlorobenzoic acid. This material was used in the subsequent step without further purification.

NMR Spectrum: ¹H NMR (399.902 MHz, CDCl₃) δ 1.23 (d, 3H), 3.17-3.25 (m, 1H), 3.46-3.54 (m, 1H), 3.62-3.67 (m, 1H), 3.74 (d, 1H), 3.93-4.01 (m, 2H), 4.20 (s, 1H), 4.29-4.38 (m, 2H), 6.60 (s, 1H), 6.99-7.02 (m, 1H), 7.20 (d, 1H), 7.47-7.51 (m, 1H), 8.40-8.42 (m, 1H).

LCMS Spectrum: m/z (ESI+)(M+H)+=337.5; HPLC tR=2.19 min.

The preparation of 2-chloro-4-(iodomethyl)-6-[(3S)-3-methylmorpholin-4-yl]pyrimidine was described earlier.

### EXAMPLE 29

### [4-[4-[(3S)-3-Methylmorpholin-4-yl]-6-(1-methylsulfonylcyclopropyl)pyrimidin-2-yl]phenyl]urea

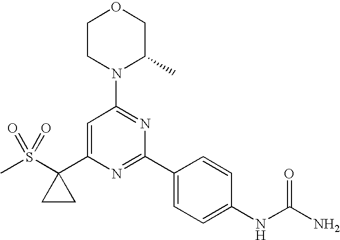

6-Aminopyridin-2(1H)-one hydrochloride salt (0.218 g, 1.49 mmol) was added to phenyl N-[4-[4-[(3S)-3-methylmorpholin-4-yl]-6-(1-methylsulfonylcyclopropyl)pyrimidin-2-yl]phenyl]carbamate (0.151 g, 0.30 mmol) and triethylamine (0.249 mL, 1.78 mmol) in DMF (2 mL) at RT. The resulting solution was stirred at 50° C. for 1 day. The solution was cooled and purified by preparative HPLC, using decreasingly polar mixtures of water (containing 1% ammonia) and acetonitrile as eluents, to give the desired material as a white solid (0.094 g).

NMR Spectrum: ¹H NMR (400.132 MHz, DMSO-d₆) δ 1.22-1.25(3H, d), 1.55-1.58(2H, q), 1.66-1.69(2H, q), 3.17-3.26(1H, td), 3.30(3H, s), 3.45-3.52(1H, td), 3.62-3.65(1H, dd), 3.75-3.78(1H, d), 3.96-3.99(1H, dd), 4.20-4.23(1H, d), 4.57(1H, bs), 5.91(2H, s), 6.77(1H, s), 7.50-7.52(2H, q), 8.19-8.21(2H, q), 8.76(1H, s).

LCMS Spectrum: m/z (ES+)(M+H)+=432; HPLC tR=1.66 min.

mTOR Kinase Assay (Echo): 0.0042 μM

The preparation of phenyl N-[4-[4-[(3S)-3-methylmorpholin-4-yl]-6-(1-methylsulfonylcyclopropyl)pyrimidin-2-yl]phenyl]carbamate was described earlier.

### EXAMPLE 30

### 3-Cyclopropyl-1-[4-[4-[4-(3,5-difluorophenyl)sulfonylpiperidin-4-yl]-6-[(3S)-3-methylmorpholin-4-yl]pyrimidin-2-yl]phenyl]urea

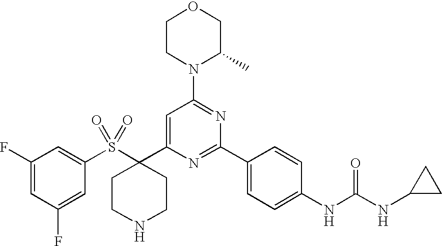

1-Chloroethyl chloroformate (0.015 mL, 0.14 mmol) and 1-[4-[4-[1-benzyl-4-(3,5-difluorophenyl)sulfonylpiperidin-4-yl]-6-[(3S)-3-methylmorpholin-4-yl]pyrimidin-2-yl]phenyl]-3-cyclopropylurea (50 mg, 0.07 mmol) were dissolved in DCM (1 mL) and sealed into a microwave tube. The reaction was heated to 110° C. for 5 minutes in a microwave reactor and then cooled to RT. Methanol (1 mL) was added and the mixture was heated to 110° C. for 5 minutes in a microwave reactor and then cooled to RT. The mixture was purified by ion exchange chromatography using an SCX column, eluting with methanol followed by 2M ammonia in methanol. The isolated material was further purified by chromatography on silica, eluting with 2% methanol in DCM to 20% methanol in DCM containing 1% ammonia. The isolated material was triturated with diethyl ether to give the desired material as a colourless solid (26 mg).

NMR Spectrum: ¹H NMR (399.9 MHz, CDCl₃) δ 0.68-0.72 (2H, m), 0.86-0.90 (2H, m), 1.36 (3H, d), 2.27-2.36 (2H, m), 2.52-2.65 (3H, m), 2.70-2.79(2H, m), 3.10-3.15 (2H, m), 3.34 (1H, dt), 3.64 (1H, dt), 3.77-3.87 (2H, m), 4.05-4.18 (2H, m), 4.43-4.52 (1H, m), 4.91 (1H, s), 6.65 (1H, s), 6.81-6.87 (1H, m), 6.95-7.02 (3H, m), 7.39 (2H, d), 7.93 (2H, d).

LCMS Spectrum: m/z (ESI+)(M+H)+=613.5; HPLC tR=2.16 min.

mTOR Kinase Assay (Echo): 0.025 μM

The preparation of 1-[4-[4-[1-benzyl-4-(3,5-difluorophenyl)sulfonylpiperidin-4-yl]-6-[(3S)-3-methylmorpholin-4-yl]pyrimidin-2-yl]phenyl]-3-cyclopropylurea was described earlier.

### EXAMPLE 31

### 1-[4-[4-[1-(3-Hydroxypropylsulfonyl)cyclopropyl]-6-[(3S)-3-methylmorpholin-4-yl]pyrimidin-2-yl]phenyl]-3-propan-2-ylurea

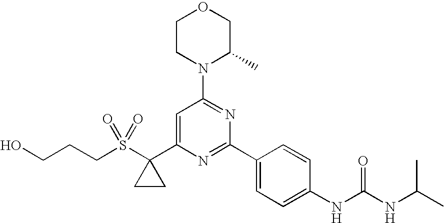

Triethylamine (0.126 mL, 0.90 mmol) was added to phenyl N-[4-[4-[1-(3-hydroxypropylsulfonyl)cyclopropyl]-6-[(3S)-3-methylmorpholin-4-yl]pyrimidin-2-yl]phenyl]carbamate (100 mg, 0.18 mmol) and propan-2-amine (0.078 mL, 0.90 mmol) in NMP (2 mL) at RT under air. The resulting solution was stirred at RT for 1 hour. The crude product was purified by preparative, using decreasingly polar mixtures of water (containing 1% ammonia) and acetonitrile as eluents, to give the desired material as a white solid (65 mg).

NMR Spectrum: ¹H NMR (400.132 MHz, DMSO-d₆) δ 1.11 (6H, d), 1.22 (3H, d), 1.54-1.58 (2H, m), 1.60-1.64 (2H, m), 1.90-1.97 (2H, m), 3.16-3.23 (1H, m), 3.31-3.34 (1H, m), 3.44-3.55 (4H, m), 3.61-3.64 (1H, m), 3.73-3.81 (2H, m), 3.95-3.99 (1H, m), 4.16-4.26 (1H, m), 4.52-4.59 (1H, m), 4.73 (1H, t), 6.08 (1H, d), 6.77 (1H, s), 7.47 (2H, d), 8.20 (2H, d), 8.57 (1H, s)

LCMS Spectrum: m/z (ESI+)(M+H)+=518; HPLC tR=2.04 min.

mTOR Kinase Assay (Echo): 0.00452 μM

The compounds below were prepared in an analogous fashion from phenyl N-[4-[4-[1-(3-hydroxypropylsulfonyl)cyclopropyl]-6-[(3S)-3-methylmorpholin-4-yl]pyrimidin-2-yl]phenyl]carbamate and the appropriate amine.

| | | | | Retention |
|---|---|---|---|---|
| Ex- | | | LCMS | time |
| ample | Structure | NAME | MH+ | (min) |
| 31a | 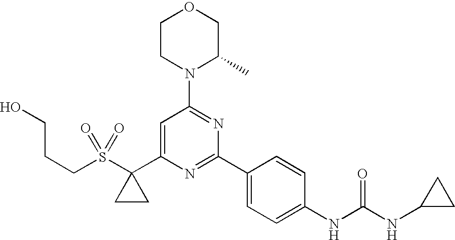 | 3-cyclopropyl-1-[4-[4-[1-(3-hydroxypropylsulfonyl)cyclopropyl]-6-[(3S)-3-methylmorpholin-4-yl]pyrimidin-2-yl]phenyl]urea | 516 | 1.74 |
| 31b | 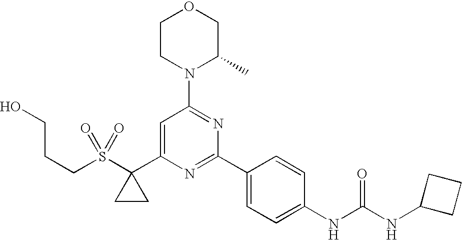 | 3-cyclobutyl-1-[4-[4-[1-(3-hydroxypropylsulfonyl)cyclopropyl]-6-[(3S)-3-methylmorpholin-4-yl]pyrimidin-2-yl]phenyl]urea | 530 | 1.94 |
| 31c | 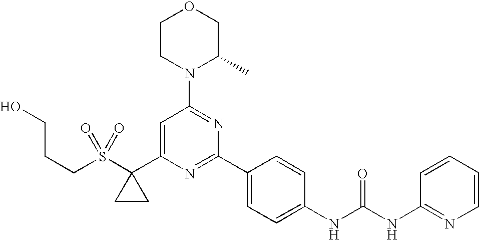 | 1-[4-[4-[1-(3-hydroxypropylsulfonyl)cyclopropyl]-6-[(3S)-3-methylmorpholin-4-yl]pyrimidin-2-yl]phenyl]-3-pyridin-2-ylurea | 553 | 2.10 |
| 31d | 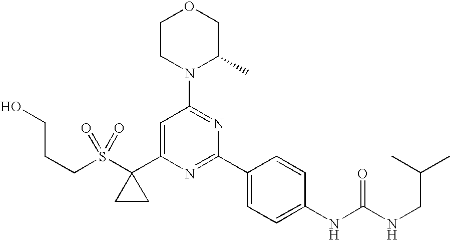 | 1-[4-[4-[1-(3-hydroxypropylsulfonyl)cyclopropyl]-6-[(3S)-3-methylmorpholin-4-yl]pyrimidin-2-yl]phenyl]-3-(2-methylpropyl)urea | 532 | 2.02 |
| 31e | 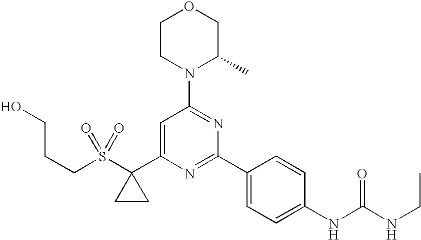 | 1-ethyl-3-[4-[4-[1-(3-hydroxypropylsulfonyl)cyclopropyl]-6-[(3S)-3-methylmorpholin-4-yl]pyrimidin-2-yl]phenyl]urea | 504 | 1.71 |
| 31f | 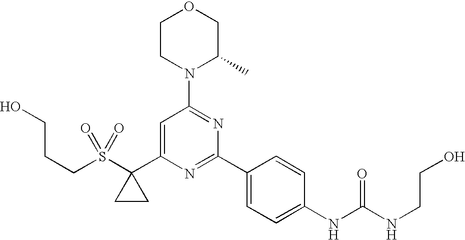 | 3-(2-hydroxyethyl)-1-[4-[4-[1-(3-hydroxypropylsulfonyl)cyclopropyl]-6-[(3S)-3-methylmorpholin-4-yl]pyrimidin-2-yl]phenyl]urea | 520 | 1.49 |
| 31g | 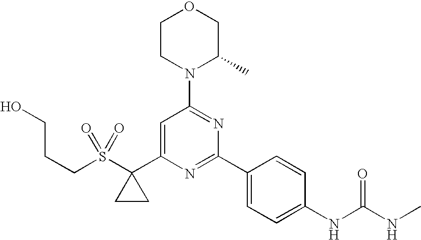 | 1-[4-[4-[1-(3-hydroxypropylsulfonyl)cyclopropyl]-6-[(3S)-3-methylmorpholin-4-yl]pyrimidin-2-yl]phenyl]-3-methylurea | 490 | 1.59 |
| 31h | 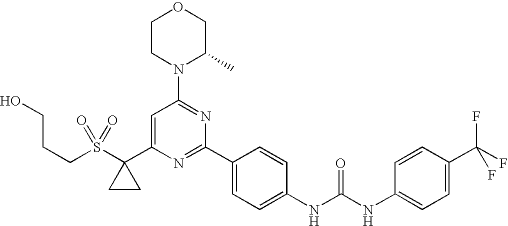 | 1-[4-[4-[1-(3-hydroxypropylsulfonyl)cyclopropyl]-6-[(3S)-3-methylmorpholin-4-yl]pyrimidin-2-yl]phenyl]-3-[4-(trifluoromethyl)phenyl]urea | 620 | 2.54 |
| 31i | 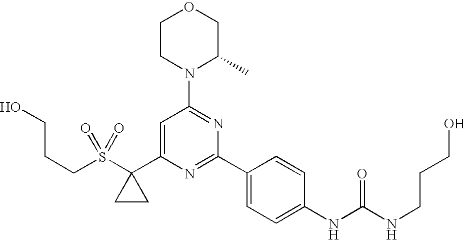 | 3-(3-hydroxypropyl)-1-[4-[4-[1-(3-hydroxypropylsulfonyl)cyclopropyl]-6-[(3S)-3-methylmorpholin-4-yl]pyrimidin-2-yl]phenyl]urea | 534 | 1.52 |
| 31j | 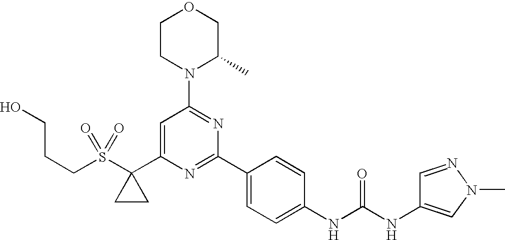 | 1-[4-[4-[1-(3-hydroxypropylsulfonyl)cyclopropyl]-6-[(3S)-3-methylmorpholin-4-yl]pyrimidin-2-yl]phenyl]-3-(1-methylpyrazol-4-yl)urea | 556 | 1.66 |
| 31k | 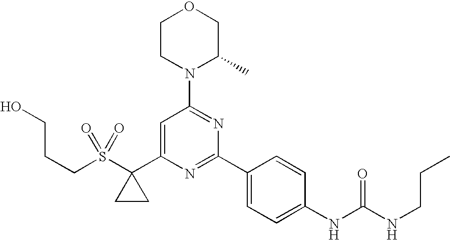 | 3-[4-[4-[1-(3-hydroxypropylsulfonyl)cyclopropyl]-6-[(3S)-3-methylmorpholin-4-yl]pyrimidin-2-yl]phenyl]-1-propylurea | 518 | 1.86 |
| 31l | 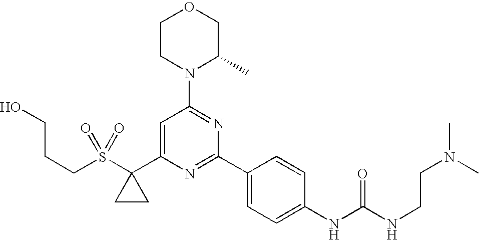 | 3-(2-dimethylaminoethyl)-1-[4-[4-[1-(3-hydroxypropylsulfonyl)cyclopropyl]-6-[(3S)-3-methylmorpholin-4-yl]pyrimidin-2-yl]phenyl]urea | 547 | 1.66 |
| 31m | 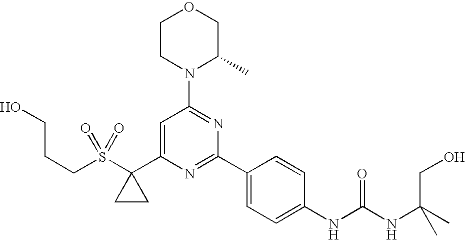 | 3-(1-hydroxy-2-methylpropan-2-yl)-1-[4-[4-[1-(3-hydroxypropylsulfonyl)cyclopropyl]-6-[(3S)-3-methylmorpholin-4-yl]pyrimidin-2-yl]phenyl]urea | 548 | 1.72 |

### EXAMPLE 31a

¹H NMR (400.132 MHz, DMSO-d₆) δ 0.40-0.43 (2H, m), 0.62-0.67 (2H, m), 1.22 (3H, d), 1.54-1.58 (2H, m), 1.60-1.64 (2H, m), 1.90-1.97 (2H, m), 2.53-2.58 (1H, m), 3.16-3.22 (1H, m), 3.44-3.55 (5H, m), 3.61-3.64 (1H, m), 3.76 (1H, d), 3.95-3.98 (1H, m), 4.17-4.25 (1H, m), 4.53-4.59 (1H, m), 4.73 (1H, t), 6.46 (1H, s), 6.77 (1H, s), 7.50 (2H, d), 8.21 (2H, d), 8.58 (1H, s).

mTOR Kinase Assay (Echo): 0.00265 μM

### EXAMPLE 31b

¹H NMR (400.132 MHz, DMSO-d₆) δ 1.22 (3H, d), 1.54-1.63 (6H, m), 1.81-1.97 (4H, m), 2.17-2.23 (2H, m), 3.17-3.22 (1H, m), 3.45-3.54 (5H, m), 3.61-3.63 (1H, m), 3.76 (1H, d), 3.96-3.98 (1H, m), 4.10-4.24 (2H, m), 4.51-4.58 (1H, m), 4.73 (1H, t), 6.48 (1H, d), 6.77 (1H, s), 7.47 (2H, d), 8.20 (2H, d), 8.60 (1H, s).

mTOR Kinase Assay (Echo): 0.00497 μM

### EXAMPLE 31c

¹H NMR (400.132 MHz, DMSO-d₆) δ 1.24 (3H, d), 1.55-1.58 (2H, m), 1.62-1.66 (2H, m), 1.91-1.98 (2H, m), 3.18-3.25 (1H, m), 3.46-3.57 (5H, m), 3.62-3.65 (1H, m), 3.77 (1H, d), 3.96-4.00 (1H, m), 4.20-4.27 (1H, m), 4.52-4.61 (1H, m), 4.75 (1H, t), 6.80 (1H, s), 7.02-7.05 (1H, m), 7.55-7.58 (1H, m), 7.64 (2H, d), 7.75-7.79 (1H, m), 8.29-8.31 (1H, m), 8.30 (2H, d), 9.49 (1H, s), 10.63 (1H, s).

mTOR Kinase Assay (Echo): 0.00131 μM

### EXAMPLE 31d

¹H NMR (400.132 MHz, DMSO-d₆) δ 0.88 (6H, d), 1.22 (3H, d), 1.54-1.56 (2H, m), 1.60-1.64 (2H, m), 1.90-1.97 (2H, m), 2.94 (2H, t), 3.16-3.23 (1H, m), 3.45-3.55 (6H, m), 3.61-3.64 (1H, m), 3.76 (1H, d), 3.96-3.98 (1H, m), 4.16-4.26 (1H, m), 4.52-4.59 (1H, m), 4.73 (1H, t), 6.26 (1H, t), 6.77 (1H, s), 7.48 (2H, d), 8.21 (2H, d), 8.68 (1H, s).

mTOR Kinase Assay (Echo): 0.00955 μM

### EXAMPLE 31e

¹H NMR (400.132 MHz, DMSO-d₆) δ 1.06 (3H, t), 1.22 (3H, d), 1.54-1.58 (2H, m), 1.60-1.64 (2H, m), 1.90-1.97 (2H, m), 3.09-3.23 (3H, m), 3.44-3.55 (5H, m), 3.61-3.64 (1H, m), 3.76 (1H, d), 3.95-3.99 (1H, m), 4.18-4.24 (1H, m), 4.53-4.59 (1H, m), 4.73 (1H, t), 6.18 (1H, t), 6.77 (1H, s), 7.49 (2H, d), 8.20 (2H, d), 8.70 (1H, s).

mTOR Kinase Assay (Echo): 0.00107 μM

### EXAMPLE 31f

¹H NMR (400.132 MHz, DMSO-d₆) δ 1.22 (3H, d), 1.54-1.64 (4H, m), 1.90-1.97 (2H, m), 3.15-3.19 (2H, m), 3.43-3.54 (8H, m), 3.61-3.63 (1H, m), 3.76 (1H, d), 3.95-3.98 (1H, m), 4.17-4.24 (1H, m), 4.51-4.59 (1H, m), 4.73-4.79 (2H, m), 6.28 (1H, t), 6.76 (1H, s), 7.48 (2H, d), 8.21 (2H, d), 8.84 (1H, s).

mTOR Kinase Assay (Echo): 0.0016 μM

### EXAMPLE 31g

¹H NMR (400.132 MHz, DMSO-d₆) δ 1.22 (3H, d), 1.54-1.58 (2H, m), 1.60-1.64 (2H, m), 1.90-1.97 (2H, m), 2.66 (3H, d), 3.16-3.23 (1H, m), 3.44-3.55 (5H, m), 3.61-3.64 (1H, m), 3.76 (1H, d), 3.95-3.99 (1H, m), 4.17-4.23 (1H, m), 4.52-4.59 (1H, m), 4.73 (1H, t), 6.07-6.10 (1H, m), 6.77 (1H, s), 7.50 (2H, d), 8.20 (2H, d), 8.78 (1H, s).

mTOR Kinase Assay (Echo): 0.00323 μM

### EXAMPLE 31h

¹H NMR (400.132 MHz, DMSO-d₆) δ 1.24 (3H, d), 1.55-1.59 (2H, m), 1.62-1.65 (2H, m), 1.91-1.98 (2H, m), 3.17-3.25 (1H, m), 3.45-3.56 (5H, m), 3.62-3.65 (1H, m), 3.77 (1H, d), 3.96-4.00 (1H, m), 4.18-4.26 (1H, m), 4.53-4.61 (1H, m), 4.75 (1H, t), 6.80 (1H, s), 7.58 (2H, d), 7.64-7.70 (4H, m), 8.29 (2H, d), 9.09 (1H, s), 9.17 (1H, s).

mTOR Kinase Assay (Echo): 0.000209 μM

### EXAMPLE 31i

¹H NMR (400.132 MHz, DMSO-d₆) δ 1.22 (3H, d), 1.54-1.64 (6H, m), 1.90-1.97 (2H, m), 3.14-3.23 (3H, m), 3.44-3.55 (7H, m), 3.61-3.64 (1H, m), 3.76 (1H, d), 3.95-3.99 (1H, m), 4.17-4.25 (1H, m), 4.52 (1H, t), 4.53-4.59 (1H, m), 4.73 (1H, t), 6.22 (1H, t), 6.77 (1H, s), 7.49 (2H, d), 8.20 (2H, d), 8.75 (1H, s).

mTOR Kinase Assay (Echo): 0.00326 μM

### EXAMPLE 31j

¹H NMR (400.132 MHz, DMSO-d₆) δ 1.23 (3H, d), 1.54-1.58 (2H, m), 1.61-1.64 (2H, m), 1.90-1.98 (2H, m), 3.16-3.24 (1H, m), 3.46-3.56 (4H, m), 3.61-3.65 (1H, m), 3.79 (3H, s), 3.96-3.99 (1H, m), 4.18-4.25 (1H, m), 4.53-4.58 (1H, m), 4.74 (1H, t), 6.78 (1H, s), 7.39 (1H, s), 7.54 (2H, d), 7.77 (1H, s), 8.24 (2H, d), 8.43 (1H, s), 8.89 (1H, s).

mTOR Kinase Assay (Echo): 0.00103 μM

### EXAMPLE 31k

¹H NMR (400.132 MHz, DMSO-d₆) δ 0.88 (3H, t), 1.22 (3H, d), 1.41-1.50 (2H, m), 1.54-1.58 (2H, m), 1.60-1.64 (2H, m), 1.88-1.97 (2H, m), 3.03-3.08 (2H, m), 3.16-3.23 (1H, m), 3.44-3.55 (5H, m), 3.61-3.64 (1H, m), 3.76 (1H, d), 3.95-3.99 (1H, m), 4.17-4.24 (1H, m), 4.51-4.59 (1H, m), 4.73 (1H, t), 6.22 (1H, t), 6.77 (1H, s), 7.49 (2H, d), 8.20 (2H, d), 8.69 (1H, s).

mTOR Kinase Assay (Echo): 0.00191 μM

### EXAMPLE 31l

¹H NMR (400.132 MHz, DMSO-d₆) δ 1.22 (3H, d), 1.54-1.58 (2H, m), 1.60-1.64 (2H, m), 1.90-1.97 (2H, m), 2.18 (6H, s), 2.33 (2H, t), 3.17-3.23 (3H, m), 3.44-3.55 (5H, m), 3.61-3.64 (1H, m), 3.76 (1H, d), 3.95-3.99 (1H, m), 4.17-4.25 (1H, m), 4.50-4.60 (1H, m), 4.73 (1H, t), 6.17 (1H, t), 6.77 (1H, s), 7.48 (2H, d), 8.20 (2H, d), 8.92 (1H, s).

mTOR Kinase Assay (Echo): 0.214 μM

### EXAMPLE 31m

¹H NMR (400.132 MHz, DMSO-d₆) δ 1.24 (6H, s), 1.54-1.58 (2H, m), 1.60-1.64 (2H, m), 1.90-1.97 (2H, m), 3.16-3.23 (1H, m), 3.38 (2H, d), 3.44-3.55 (5H, m), 3.61-3.64 (1H, m), 3.76 (1H, d), 3.95-3.99 (1H, m), 4.17-4.25 (1H, m), 4.51-4.59 (1H, m), 4.74 (1H, t), 4.99 (1H, t), 6.01 (1H, s), 6.76 (1H, s), 7.45 (2H, d), 8.19 (2H, d), 8.77 (1H, s).

mTOR Kinase Assay (Echo): 0.00591 μM

The preparation of phenyl N-[4-[4-[1-(3-hydroxypropylsulfonyl)cyclopropyl]-6-[(3S)-3-methylmorpholin-4-yl]pyrimidin-2-yl]phenyl]carbamate is described below:

### Phenyl N-[4-[4-[1-(3-hydroxypropylsulfonyl)cyclopropyl]-6-[(3S)-3-methylmorpholin-4-yl]pyrimidin-2-yl]phenyl]carbamate

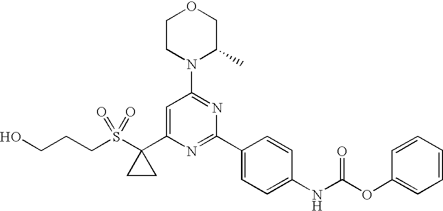

Phenyl chloroformate (0.581 mL, 4.63 mmol) was added to 3-[1-[2-(4-aminophenyl)-6-[(3S)-3-methylmorpholin-4-yl]pyrimidin-4-yl]cyclopropyl]sulfonylpropan-1-ol (1.82 g, 4.21 mmol) and sodium bicarbonate (0.530 g, 6.31 mmol) in dioxane (100 mL) at 5° C. under a nitrogen atmosphere. The resulting suspension was stirred overnight and allowed to come to RT. The reaction mixture was diluted with ethyl acetate (200 mL), and washed sequentially with water (100 mL) and saturated brine (100 mL). The organic layer was dried (MgSO₄), filtered and evaporated to afford desired product as a gum. The gum was triturated with a mixture of diethyl ether and isohexane to give the desired material as a white solid (2.32 g).

NMR Spectrum: ¹H NMR (400.132 MHz, DMSO-d₆) δ 1.17 (3H, d), 1.48-1.52 (2H, m), 1.55-1.61 (2H, m), 1.83-1.90 (2H, m), 3.11-3.18 (1H, m), 3.38-3.46 (4H, m), 3.54-3.58 (1H, m), 3.69 (1H, d), 3.75-3.94 (2H, m), 3.88-3.92 (1H, m), 4.14-4.18 (1H, m), 4.47-4.53 (1H, m), 6.75 (1H, s), 7.17-7.23 (3H, m), 7.36-7.39 (2H, m), 7.56 (2H, d), 8.23 (2H, d), 10.37 (1H, s)

LCMS Spectrum: m/z (ESI+)(M+H)+=553; HPLC tR=2.51 min.

### 3-[1-[2-(4-Aminophenyl)-6-[(3S)-3-methylmorpholin-4-yl]pyrimidin-4-yl]cyclopropyl]sulfonylpropan-1-ol

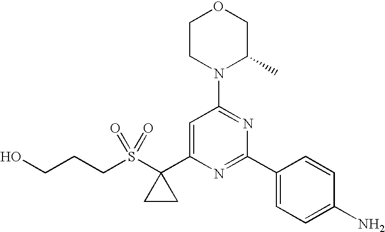

Dichlorobis(triphenylphosphine)palladium (II) (0.218 g, 0.31 mmol) was added to 3-[1-[2-chloro-6-[(3S)-3-methylmorpholin-4-yl]pyrimidin-4-yl]cyclopropyl]sulfonylpropan-1-ol (2.33 g, 6.20 mmol), 4-(4,4,5,5-tetramethyl-1,3,2-dioxaborolan-2-yl)aniline (1.766 g, 8.06 mmol) and 2M aqueous sodium carbonate solution (11.16 mL, 22.32 mmol) in DMF (15 mL), water (37.5 mL), ethanol (15 mL) and DME (15 mL) at RT under a nitrogen atmosphere. The resulting solution was stirred at 80° C. for 17 hours. The reaction mixture was diluted with ethyl acetate (100 mL), and washed sequentially with water (100 mL) and saturated brine (100 mL). The organic layer was dried (MgSO₄), filtered and evaporated to afford crude product as a brown solid. The crude product was purified by flash silica chromatography, eluting with 0 to 75% ethyl acetate in DCM, to give the desired material as a cream solid (1.82 g).

NMR Spectrum: ¹H NMR (400.132 MHz, DMSO-d₆) δ 1.22 (3H, d), 1.52-1.54 (2H, m), 1.60-1.62 (2H, m), 1.90-1.97 (2H, m), 3.14-3.21 (1H, m), 3.44-3.52 (5H, m), 3.60-3.64 (1H, m), 3.75 (1H, d), 3.94-3.98 (1H, m), 4.16-4.19 (1H, m), 4.48-4.55 (1H, m), 4.67 (1H, t), 5.56 (2H, s), 6.60 (2H, d), 6.67 (1H, s), 8.04 (2H, d)

LCMS Spectrum: m/z (ESI+)(M+H)+=433; HPLC tR=1.81 min.

### 3-[1-[2-Chloro-6-[(3S)-3-methylmorpholin-4-yl]pyrimidin-4-yl]cyclopropyl]sulfonylpropan-1-ol

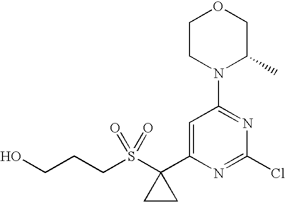

Tetrabutylammonium fluoride (1M in THF, 31 mL, 31 mmol) was added to 3-[1-[2-chloro-6-[(3S)-3-methylmorpholin-4-yl]pyrimidin-4-yl]cyclopropyl]sulfonylpropoxy-tri(propan-2-yl)silane (3.28 g, 6.16 mmol) in THF (30 mL) at RT. The resulting solution was stirred at RT for 1 hour then concentrated in vacuo and diluted with saturated ammonium chloride (10 mL) and water. The mixture was extracted twice with ethyl acetate, the combined organic extracts washed with brine, dried (MgSO₄), filtered and evaporated to give the desired material as a gum (2.33 g).

NMR Spectrum: ¹H NMR (400.132 MHz, CDCl₃) δ 1.33 (3H, d), 1.42-1.51 (4H, m), 2.07-2.14 (2H, m), 2.40 (1H, s), 3.28-3.32 (2H, m), 3.37-3.42 (3H, m), 3.51-3.57 (1H, m), 3.66-3.70 (1H, m), 3.77-3.80 (2H, m), 3.99-4.02 (1H, m), 4.28-4.38 (1H, m), 6.84 (1H, s)

LCMS Spectrum: m/z (ESI+)(M+H)+=376; HPLC tR=1.58 min.

### 3-[1-[2-Chloro-6-[(3S)-3-methylmorpholin-4-yl]pyrimidin-4-yl]cyclopropyl]sulfonylpropoxy-tri(propan-2-yl)silane

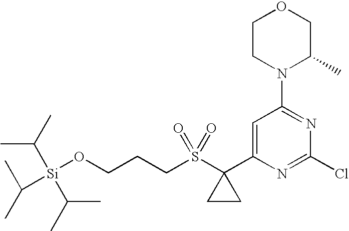

1,2-dibromoethane (1.723 mL, 20 mmol) was added to 3-[[2-chloro-6-[(3S)-3-methylmorpholin-4-yl]pyrimidin-4-yl]methylsulfonyl]propoxy-tri(propan-2-yl)silane (5.0 g, 9.88 mmol), 40% sodium hydroxide solution (10 mL, 98.78 mmol) and tetrabutylammonium bromide (0.645 g, 2 mmol) in toluene (50 mL) at RT under air. The resulting mixture was stirred at 60° C. for 4 hours. The reaction mixture was evaporated to dryness and redissolved in ethyl acetate (100 mL) and washed sequentially with water (100 mL) and saturated brine (100 mL). The organic layer was dried (MgSO₄), filtered and evaporated to afford crude product. The crude product was purified by flash silica chromatography, eluting with 0 to 20% ethyl acetate in DCM, to give the desired material as a colourless gum (2.86 g).

NMR Spectrum: ¹H NMR (400.132 MHz, CDCl₃) δ 1.00-1.05 (21H, m), 1.32 (3H, d), 1.49-1.52 (2H, m), 1.78-1.81 (2H, m), 2.02-2.09 (2H, m), 3.21-3.32 (3H, m), 3.50-3.56 (1H, m), 3.65-3.69 (1H, m), 3.77-3.80 (3H, m), 3.98-4.02 (2H, m), 4.28-4.36 (1H, m), 6.90 (1H, s)

LCMS Spectrum: m/z (ESI+)(M+H)+=532; HPLC tR=3.37 min.

### 3-[[2-Chloro-6-[(3S)-3-methylmorpholin-4-yl]pyrimidin-4-yl]methylsulfonyl]propoxy-tri(propan-2-yl)silane

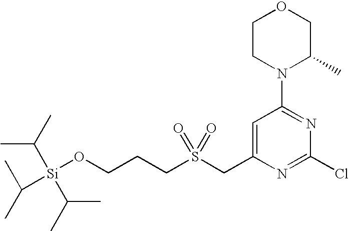

3-[[2-Chloro-6-[(3S)-3-methylmorpholin-4-yl]pyrimidin-4-yl]methylsulfonyl]propan-1-ol (5.04 g, 14.41 mmol) in DMF (25 mL) was added to chlorotrisopropylsilane (3.70 mL, 17.29 mmol) and imidazole (2.354 g, 34.58 mmol) in DMF (25 mL) at RT over a period of 5 minutes under a nitrogen atmosphere. The resulting solution was stirred at RT for 18 hours. The reaction mixture was evaporated to dryness and redissolved in DCM (200 mL) then washed sequentially with water (100 mL) and saturated brine (100 mL). The organic layer was dried (MgSO₄), filtered and evaporated to give the desired material as an oil (7.29 g).

NMR Spectrum: ¹H NMR (400.132 MHz, CDCl₃) δ 0.99-1.07 (21H, m), 1.33 (3H, d), 2.06-2.13 (2H, m), 3.20-3.24 (2H, m), 3.26-3.34 (1H, m), 3.50-3.57 (1H, m), 3.66-3.70 (1H, m), 3.77-3.83 (3H, m), 3.99-4.03 (2H, m), 4.16 (2H, s), 4.25-4.37 (1H, m), 6.54 (1H, s)

LCMS Spectrum: m/z (ESI+)(M+H)+=506; HPLC tR=3.42 min.

### 3-[[2-Chloro-6-[(3S)-3-methylmorpholin-4-yl]pyrimidin-4-yl]methylsulfonyl]propan-1-ol

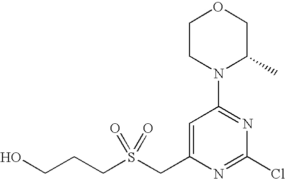

3-Chlorobenzoperoxoic acid (4.00 g, 23.16 mmol) was added to 3-[[2-chloro-6-[(3S)-3-methylmorpholin-4-yl]pyrimidin-4-yl]methylsulfanyl]propan-1-ol (3.68 g, 11.58 mmol) in DCM (100 mL) at RT over a period of 5 minutes. The resulting solution was stirred at RT for 3 hours. A further portion of 3-chlorobenzoperoxoic acid (2.00 g, 11.58 mmol) was added and the resulting solution was stirred at RT for an additional 1 hour. The reaction mixture was washed sequentially with 10% aqueous sodium metabisulphite solution (2×100 mL), a saturated aqueous solution of sodium hydrogen carbonate (100 mL), and saturated brine (100 mL). The organic layer was dried (MgSO₄), filtered and evaporated to give the desired material as a gum (4.05 g).

NMR Spectrum: ¹H NMR (400.132 MHz, CDCl₃) δ 1.34 (3H, d), 2.12-2.18 (2H, m), 3.27 (2H, t), 3.31-3.35 (1H, m), 3.51-3.57 (1H, m), 3.67-3.70 (1H, m), 3.77-3.82 (3H, m), 3.99-4.03 (1H, m), 4.18 (2H, s), 4.26-4.37 (1H, m), 6.51 (1H, s)

LCMS Spectrum: m/z (ESI+)(M+H)+=350; HPLC tR=1.30 min.

### 3-[[2-Chloro-6-[(3S)-3-methylmorpholin-4-yl]pyrimidin-4-yl]methylsulfanyl]propan-1-ol

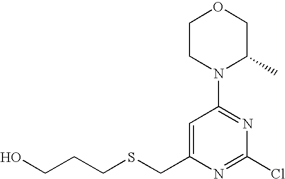

A solution of 2-chloro-4-(iodomethyl)-6-[(3S)-3-methylmorpholin-4-yl]pyrimidine (12.4 g, 35.07 mmol) in DCM (50 mL) was added to a stirred solution of 3-mercapto-1-propanol (3.64 mL, 42.08 mmol) and DIPEA (9.77 mL, 56.11 mmol) in DCM (100 mL) at RT, over a period of 40 minutes under a nitrogen atmosphere. The resulting solution was stirred at RT for 18 hours. The reaction mixture was washed sequentially with a saturated aqueous solution of sodium hydrogen carbonate (2×50 mL) and saturated brine (50 mL). The organic layer was dried (MgSO₄), filtered and evaporated to afford crude product as a dark brown oil. The crude product was purified by flash silica chromatography, eluting with 0 to 75% ethyl acetate in DCM, to give the desired material as a yellow gum (5.86 g).

NMR Spectrum: ¹H NMR (400.132 MHz, CDCl₃) δ 1.32 (3H, d), 1.84-1.90 (2H, m), 1.94 (1H, s), 2.69 (2H, t), 3.24-3.32 (1H, m), 3.51-3.58 (1H, m), 3.61 (2H, s), 3.67-3.71 (1H, m), 3.73-3.80 (3H, m), 3.98-4.04 (2H, m), 4.28-4.34 (1H, m), 6.45 (1H, s)

LCMS Spectrum: m/z (ESI+)(M+H)+=318; HPLC tR=1.55 min.

The preparation of 2-chloro-4-(iodomethyl)-6-[(3S)-3-methylmorpholin-4-yl]pyrimidine was described earlier.

### EXAMPLE 32

### N,N-Dimethyl-1-[2-[4-(methylcarbamoylamino)phenyl]-6-morpholin-4-ylpyrimidin-4-yl]cyclopropane-1-carboxamide

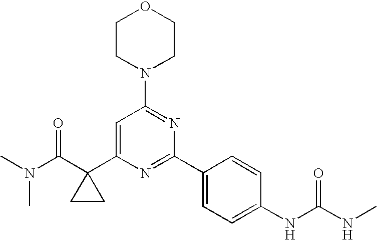

DIPEA (0.127 mL, 0.72 mmol) was added to a suspension of 1-[2-[4-(methylcarbamoylamino)phenyl]-6-morpholin-4-ylpyrimidin-4-yl]cyclopropane-1-carboxylic acid (96 mg, 0.24 mmol), dimethylamine (2.0M in THF, 0.36 mL, 0.72 mmol) and HBTU (138 mg, 0.36 mmol) in DMF (3 mL). The reactions were stirred at RT for 3 hours. The reaction mixture was passed down a SCX-2 ion exchange column, eluting with methanol followed by 7N ammonia in methanol. The isolated material was further purified by preparative HPLC, using decreasingly polar mixtures of water (containing 1% ammonia) and acetonitrile as eluents, to give the desired material as a white solid (54 mg).

NMR Spectrum: ¹H NMR (400.13 MHz, DMSO-d₆) δ 1.28-1.31 (2H, m), 1.51-1.54 (2H, m), 2.65 (3H, d), 2.87-2.89 (3H, s), 2.94 (3H, s), 3.64 (4H, m), 3.69-3.71 (4H, m), 6.08 (1H, d), 6.22 (1H, s), 7.46-7.49 (2H, m), 8.16-8.18 (2H, m), 8.76 (1H, s)

LCMS Spectrum: m/z (ESI+)(M+H)+=425; HPLC tR=1.45 min.

mTOR Kinase Assay (Echo): 0.00315 μM

The following compounds were made in an analogous fashion from 1-[2-[4-(methylcarbamoylamino)phenyl]-6-morpholin-4-ylpyrimidin-4-yl]cyclopropane-1-carboxylic acid and the appropriate amine.

| | | | LCMS | Retention |
|---|---|---|---|---|
| Example | Structure | NAME | MH+ | time (min) |
| 32a | 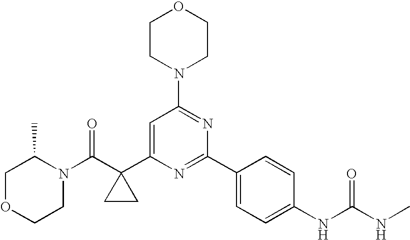 | 3-methyl-1-[4-[4-[1-[(3S)-3-methylmorpholine-4-carbonyl]cyclopropyl]-6-morpholin-4-ylpyrimidin-2-yl]phenyl]urea | 481.4 | 1.53 |
| 32b | 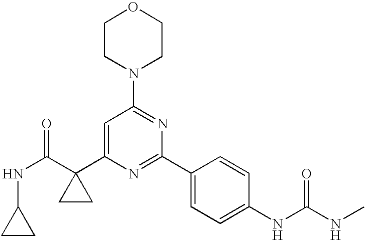 | N-cyclopropyl-1-[2-[4-(methylcarbamoylamino)phenyl]-6-morpholin-4-ylpyrimidin-4-yl]cyclopropane-1-carboxamide | 437.4 | 1.5 |
| 32c | 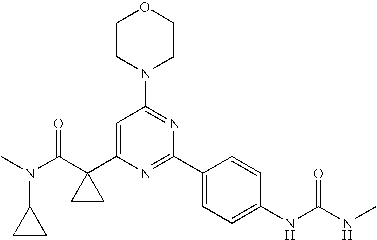 | N-cyclopropyl-N-methyl-1-[2-[4-(methylcarbamoylamino)phenyl]-6-morpholin-4-ylpyrimidin-4-yl]cyclopropane-1-carboxamide | 451.4 | 1.62 |
| 32d | 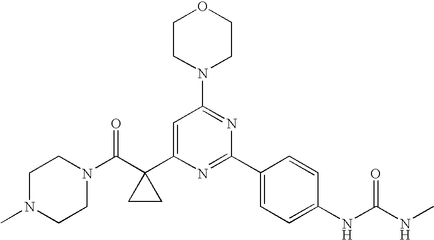 | 3-methyl-1-[4-[4-[1-(4-methylpiperazine-1-carbonyl)cyclopropyl]-6-morpholin-4-ylpyrimidin-2-yl]phenyl]urea | 480.4 | 1.35 |

### EXAMPLE 32a

¹H NMR (400.13 MHz, DMSO-d₆) δ 1.23 (3H, d), 1.31 (2H, d), 1.62 (2H, d), 2.65-2.66 (3H, m), 3.17 (1H, d), 3.48 (1H, d), 3.50 (1H, m), 3.66 (4H, s), 3.70 (4H, s), 3.70 (1H, s), 3.85 (1H, m), 4.23 (1H, d), 4.47-4.49 (1H, m), 6.07 (1H, q), 6.23 (1H, d), 7.48 (2H, d), 8.19 (2H, d), 8.76 (1H, s).

mTOR Kinase Assay (Echo): 0.0425 μM

### EXAMPLE 32b

¹H NMR (400.13 MHz, DMSO-d₆) δ 0.42-0.46 (2H, m), 0.60-0.65 (2H, m), 1.30-1.37 (4H, m), 2.66 (3H, d), 2.67-2.68 (1H, m), 3.67 (8H, s), 6.08 (1H, d), 6.46 (1H, s), 7.48-7.50 (2H, m), 8.14-8.17 (2H, m), 8.37 (1H, d), 8.77 (1H, s).

mTOR Kinase Assay (Echo): 0.00829 μM

### EXAMPLE 32c

¹H NMR (400.13 MHz, DMSO-d₆) δ 0.64 (2H, s), 0.69 (2H, s), 1.32 (2H, s), 1.49 (2H, t), 2.33 (1H, t), 2.65-2.66 (3H, m), 2.73-2.76 (3H, m), 3.64 (4H, s), 3.69-3.71 (4H, m), 6.07 (1H, q), 6.30 (1H, s), 7.46-7.49 (2H, m), 8.16-8.18 (2H, m), 8.75 (1H, s).

mTOR Kinase Assay (Echo): 0.00288 μM

### EXAMPLE 32d

¹H NMR (400.13 MHz, DMSO-d₆) δ 1.29-1.32 (2H, m), 1.50-1.53 (2H, m), 2.11-2.11 (3H, m), 2.14 (2H, s), 2.33 (2H, t), 2.65-2.66 (3H, m), 3.30 (2H, s), 3.57 (2H, s), 3.67-3.70 (8H, m) 6.08 (1H, q), 6.26 (1H, s), 7.46-7.50 (2H, m), 8.17-8.20 (2H, m), 8.76 (1H, s).

mTOR Kinase Assay (Echo): 0.0131 μM

The preparation of 1-[2-[4-(methylcarbamoylamino)phenyl]-6-morpholin-4-ylpyrimidin-4-yl]cyclopropane-1-carboxylic acid is described below:

### 1-[2-[4-(Methylcarbamoylamino)phenyl]-6-morpholin-4-ylpyrimidin-4-yl]cyclopropane-1-carboxylic acid

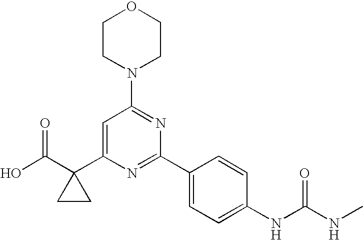

Lithium hydroxide-1-hydrate (59.5 mg, 1.42 mmol) was added in one portion to methyl 1-[2-[4-(methylcarbamoylamino)phenyl]-6-morpholin-4-ylpyrimidin-4-yl]cyclopropane-1-carboxylate (583 mg, 1.42 mmol) in methanol (8 mL) and water (8 mL). The resulting suspension was stirred at 60° C. for 5 hours. The reaction mixture was evaporated to dryness, redissolved in water (25 mL), and washed with diethyl ether (25 mL). The aqueous layer was acidified with 2M hydrochloric acid and the precipitate was collected by filtration, washed with diethyl ether and dried under vacuum to give the desired material as a brown solid (486 mg).

NMR Spectrum: ¹H NMR (400.13 MHz, DMSO-d₆) δ 1.51-1.58 (4H, m), 2.65-2.66 (3H, m), 3.70 (8H, s), 6.09 (1H, q), 6.73 (1H, s), 7.49-7.52 (2H, m), 8.13-8.15 (2H, m), 8.80 (1H, s), 14.09 (1H, s)

LCMS Spectrum: m/z (ESI+) (M+H)+=398; HPLC tR=1.21 min.

### Methyl 1-[2-[4-(methylcarbamoylamino)phenyl]-6-morpholin-4-ylpyrimidin-4-yl]cyclopropane-1-carboxylate

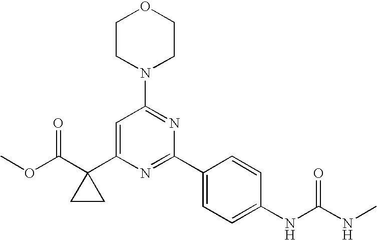

Methylamine (1.97 mL, 3.94 mmol) was added in one portion to methyl 1-[6-morpholin-4-yl-2-[4-(phenoxycarbonylamino)phenyl]pyrimidin-4-yl]cyclopropane-1-carboxylate (934 mg, 1.97 mmol) in DMF (20 mL) The resulting solution was stirred at RT for 4 hours. The reaction mixture was evaporated to dryness and the residue purified by ion exchange chromatography using an SCX column, eluting with methanol followed by 7M ammonia in methanol, to give the desired material as a yellow solid (713 mg).

NMR Spectrum: ¹H NMR (400.13 MHz, DMSO-d₆) δ 1.49-1.55 (4H, m), 2.66 (3H, d), 3.65 (3H, s), 3.69 (8H, s), 6.05 (1H, t), 6.83 (1H, s), 7.46-7.49 (2H, m), 8.16-8.19 (2H, m), 8.71 (1H, s)

LCMS Spectrum: m/z (ESI+) (M+H)+=412; HPLC tR=1.93 min.

### Methyl 1-[6-morpholin-4-yl-2-[4-(phenoxycarbonylamino)phenyl]pyrimidin-4-yl]cyclopropane-1-carboxylate

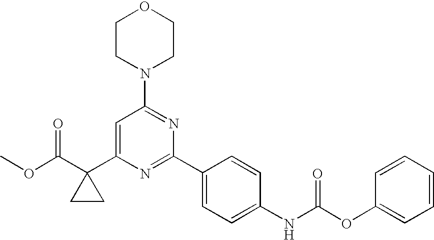

Phenyl chloroformate (0.53 mL, 4.20 mmol) was added dropwise to methyl 1-[2-(4-aminophenyl)-6-morpholin-4-ylpyrimidin-4-yl]cyclopropane-1-carboxylate (1.49 g, 4.20 mmol) and sodium bicarbonate (0.53 g, 6.31 mmol) in dioxane (50 mL) under nitrogen. The resulting suspension was stirred at RT for 2 hours. The reaction mixture was evaporated to dryness, redissolved in ethyl acetate (60 mL) and washed with water (60 mL). The organic layer was dried (MgSO₄), filtered and concentrated in vacuo to give the desired material as a yellow solid (2.1 g).

NMR Spectrum: ¹H NMR (400.13 MHz, DMSO-d₆) δ 1.52-1.54 (4H, m), 3.57 (3H, s), 3.71 (8H, s), 6.89 (1H, s), 7.24-7.30 (3H, m), 7.43-7.47 (2H, m), 7.62 (2H, d), 8.27 (2H, d), 10.46 (1H, s)

LCMS Spectrum: m/z (ESI+) (M+H)+=475; HPLC tR=2.83 min.

### Methyl 1-[2-(4-aminophenyl)-6-morpholin-4-ylpyrimidin-4-yl]cyclopropane-1-carboxylate

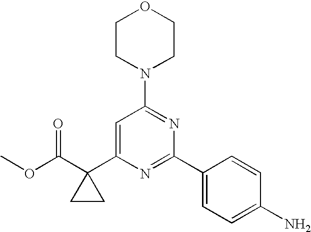

Sodium hydride (434 mg, 10.84 mmol) was added in one portion to methyl 2-[2-(4-aminophenyl)-6-morpholin-4-ylpyrimidin-4-yl]acetate (3.56 g, 10.84 mmol) in DMF (75 mL) cooled to 0° C. under nitrogen. The resulting solution was stirred at 0° C. for 10 minutes then 1,2-dibromoethane (0.981 mL, 11.38 mmol) added and the reaction stirred at 0° C. for 5 minutes. Further sodium hydride (434 mg, 10.84 mmol) was added in one portion and the reaction stirred at 0° C. for 1 hour. The reaction mixture was quenched with a saturated aqueous solution of ammonium chloride (10 mL). The reaction mixture was evaporated to dryness, redissolved in ethyl acetate (100 mL), and washed sequentially with water (100 mL) and saturated brine solution (100 mL). The organic layer was dried (MgSO₄), filtered and concentrated in vacuo. The crude residue was triturated with diethyl ether to give the desired material as a yellow solid (2.23 g).

NMR Spectrum: ¹H NMR (400.13 MHz, DMSO-d₆) δ 1.47-1.53 (4H, m), 3.64 (8H, t), 3.69-3.71 (3H, m), 5.50-5.60 (2H, s), 6.57-6.60 (2H, m), 6.73 (1H, s), 7.99-8.03 (2H, m)

LCMS Spectrum: m/z (ESI+) (M+H)+=355; HPLC tR=2.06 min.

### Methyl 2-[2-(4-aminophenyl)-6-morpholin-4-ylpyrimidin-4-yl]acetate

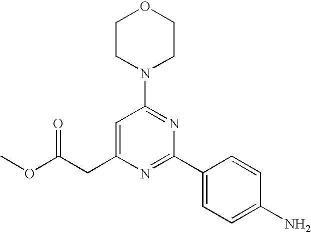

DBU (2.2 mL, 14.68 mmol) was added to methyl 2-[2-(4-aminophenyl)-6-hydroxypyrimidin-4-yl]acetate (3.46 g, 13.35 mmol) and N-phenyltrifluoromethanesulfonimide (5.24 g, 14.68 mmol) in DCM (120 mL). The resulting solution was stirred at RT for 2 hours. Morpholine (2.91 mL, 33.36 mmol) was added and the reaction stirred at RT for 2 hours. The reaction mixture was diluted with DCM (20 mL) and washed with a saturated aqueous solution of sodium hydrogen carbonate (100 mL). The organic layer was dried (MgSO4) filtered and concentrated in vacuo. The crude product was purified by flash silica chromatography, elution gradient 0 to 40% ethyl acetate in DCM, to give the desired material as a yellow solid (3.65 g).

NMR Spectrum: ¹H NMR (400.13 MHz, CDCl₃) δ 3.62 (2H, d), 3.66 (8H,m), 3.72-3.73 (3H, m), 6.27 (1H, s), 6.61-6.64 (2H, m), 7.17-7.23 (2H, m), 8.12-8.16 (2H, m)

LCMS Spectrum: m/z (ESI+) (M+H)+=329; HPLC tR=1.74 min.

### Methyl 2-[2-(4-aminophenyl)-6-hydroxypyrimidin-4-yl]acetate

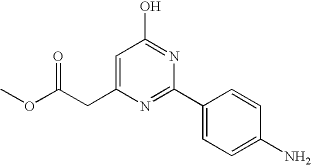

To a stirred solution of dimethyl 3-oxopentanedioate (15.06 g, 86.50 mmol) in methanol (125 mL) was added 4-aminobenzimidamide dihydrochloride (12 g, 57.67 mmol) and potassium carbonate (19.93 g, 144.17 mmol) and the reaction mixture heated at 90° C. for 5 hours then allowed to cool to RT. The mixture was concentrated in vacuo, redissolved in water (150 mL) and extracted with DCM (150 mL). The organic layer was dried (MgSO₄), filtered and concentrated in vacuo. The aqueous layer was adjusted to pH5 using acetic acid and the precipitate formed was filtered and dried in a vacuum oven to give the desired material as a yellow solid (3.49 g).

LCMS Spectrum: m/z (ES+)(M+H)+=260; HPLC tR=1.06 min.

### EXAMPLE 33

### 1-[2-[4-(Cyclopropylcarbamoylamino)phenyl]-6-morpholin-4-ylpyrimidin-4-yl]-N,N-dimethylcyclopropane-1-carboxamide

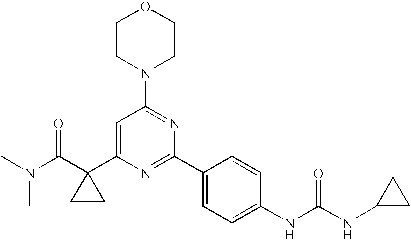

DIPEA (0.142 mL, 0.81 mmol) was added to a suspension of 1-[2-[4-(cyclopropylcarbamoylamino)phenyl]-6-morpholin-4-ylpyrimidin-4-yl]cyclopropane-1-carboxylic acid (115 mg, 0.27 mmol), dimethylamine (2.0M in THF, 0.405 mL, 0.81 mmol) and HBTU (155 mg, 0.36 mmol) in DMF (3 mL). The reactions were stirred at RT for 3 hours then purified on a ion exchange SCX-2 column, eluting with methanol followed by 7N ammonia in methanol. The residue was further purified by preparative HPLC, using decreasingly polar mixtures of water (containing 1% ammonia) and acetonitrile as eluents, to give the desired material as a white solid (20 mg).

NMR Spectrum: ¹H NMR (400.13 MHz, DMSO-d₆) δ 0.39-0.43 (2H, m), 0.62-0.67 (2H, m), 1.28-1.31 (2H, m), 1.51-1.54 (2H, m), 2.54-2.56 (1H, m), 2.87 (3H, s), 2.94 (3H, s), 3.64 (4H, d), 3.69-3.71 (4H, m), 6.22 (1H, s), 6.44 (1H, d), 7.47-7.49 (2H, m), 8.16-8.19 (2H, m), 8.55 (1H, s)

LCMS Spectrum: m/z (ESI+)(M+H)+=451; HPLC tR=1.83 min.

mTOR Kinase Assay (Echo): 0.00929 μM

The following compounds were made in an analogous fashion from 1-[2-[4-(cyclopropylcarbamoylamino)phenyl]-6-morpholin-4-ylpyrimidin-4-yl]cyclopropane-1-carboxylic acid and the appropriate amine.

| | | | LCMS | Retention |
|---|---|---|---|---|
| Example | Structure | NAME | MH+ | time (min) |
| 33a | 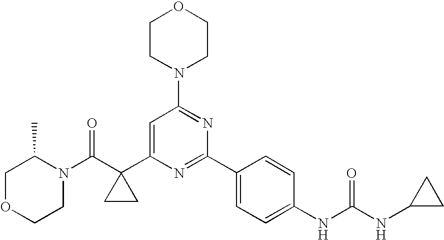 | 3-cyclopropyl-1-[4-[4-[1-[(3S)-3-methylmorpholine-4-carbonyl]cyclopropyl]-6-morpholin-4-ylpyrimidin-2-yl]phenyl]urea | 507 | 1.90 |
| 33b | 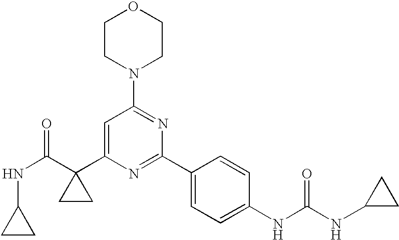 | N-cyclopropyl-1-[2-[4-(cyclopropylcarbamoylamino)phe-nyl]-6-morpholin-4-ylpyrimidin-4-yl]cyclopropane-1-carboxamide | 463 | 1.88 |
| 33c | 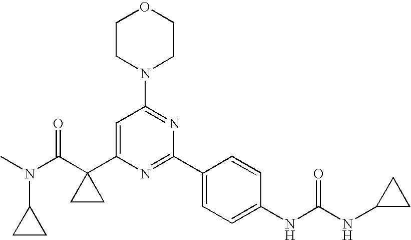 | N-cyclopropyl-1-[2-[4-(cyclopropylcarbamoylamino)phe-nyl]-6-morpholin-4-ylpyrimidin-4-yl]-N-methylcyclopropane-1-carboxamide | 477 | 2.00 |
| 33d | 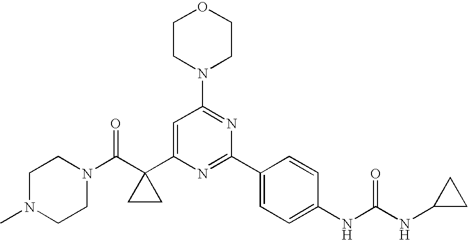 | 3-cyclopropyl-1-[4-[4-[1-(4-methylpiperazine-1-carbonyl)cyclopropyl]-6-morpholin-4-ylpyrimidin-2-yl]phenyl]urea | 506 | 1.72 |

### EXAMPLE 33a

¹H NMR (400.13 MHz, DMSO-d₆) δ 0.39-0.43 (2H, m), 0.62-0.67 (2H, m), 1.16 (1H, s), 1.23 (2H, d), 1.32-1.36 (2H, m), 1.40 (1H, d), 1.61-1.64 (2H, m), 2.52-2.57 (1H, m), 3.17 (1H, q), 3.15-3.21 (1H, m), 3.46-3.49 (1H, m), 3.66-3.66 (4H, m), 3.69 (4H, d), 3.71 (1H, s), 4.20 (1H, d), 4.48 (1H, d), 6.24 (1H, s), 6.45 (1H, d), 7.48 (2H, d), 8.19 (2H, d), 8.55 (1H, s).

mTOR Kinase Assay (Echo): 0.0584 μM

### EXAMPLE 33b

¹H NMR (400.13 MHz, DMSO-d₆) δ 0.41-0.45 (4H, m), 0.61-0.66 (4H, m), 1.31-1.35 (4H, m), 2.54-2.56 (1H, m), 2.71 (1H, d), 3.67-3.72 (8H, m), 6.45 (2H, d), 7.50 (2H, d), 8.15-8.17 (2H, m), 8.36 (1H, d), 8.56 (1H, s).

mTOR Kinase Assay (Echo): 0.012 μM

### EXAMPLE 33c

¹H NMR (400.13 MHz, DMSO-d₆) δ 0.39-0.43 (2H, m), 0.50 (2H, m), 0.63-0.65 (2H, m), 0.68 (2H, d), 1.32-1.40 (2H, m), 1.49 (2H, t), 2.54-2.57 (1H, m), 2.75 (3H, d), 2.89 (1H, s), 3.64 (4H, d), 3.69 (4H, d), 6.31 (1H, s), 6.45 (1H, d), 7.48 (2H, d), 8.18 (2H, d), 8.54 (1H, s).

mTOR Kinase Assay (Echo): 0.00258 μM

### EXAMPLE 33d

¹H NMR (400.13 MHz, DMSO-d₆) δ 0.39-0.43 (2H, m), 0.62-0.67 (2H, m), 1.30-1.32 (2H, m), 1.51 (2H, t), 2.12 (4H, s), 2.33 (3H, s), 2.53-2.56 (1H, m), 3.58 (4H, s), 3.67-3.71 (8H, d), 6.27 (1H, s), 6.45 (1H, d), 7.47-7.50 (2H, m), 8.18-8.20 (2H, m), 8.55 (1H, s).

mTOR Kinase Assay (Echo): 0.133 μM

The preparation of 1-[2-[4-(cyclopropylcarbamoylamino)phenyl]-6-morpholin-4-ylpyrimidin-4-yl]cyclopropane-1-carboxylic acid is described below:

### 1-[2-[4-(Cyclopropylcarbamoylamino)phenyl]-6-morpholin-4-ylpyrimidin-4-yl]cyclopropane-1-carboxylic acid

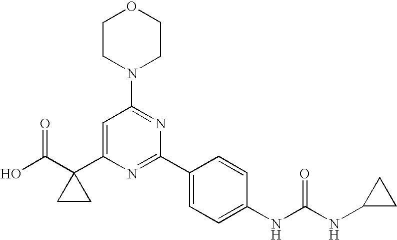

Lithium hydroxide-1-hydrate (67 mg, 1.59 mmol) was added in one portion to methyl 1-[2-[4-(cyclopropylcarbamoylamino)phenyl]-6-morpholin-4-ylpyrimidin-4-yl]cyclopropane-1-carboxylate (697 mg, 1.59 mmol) in methanol (8 mL) and water (8 mL) The resulting suspension was stirred at 80° C. for 1 hour. The reaction mixture was evaporated to dryness, redissolved in water (25 mL), and washed with diethyl ether (25 mL). The aqueous layer was acidified with 2M hydrochloric acid, the precipitate collected by filtration, washed with diethyl ether and dried under vacuum to give the desired material as a brown solid (580 mg).

NMR Spectrum: ¹H NMR (400.13 MHz, DMSO-d₆) δ 0.40-0.43 (2H, m), 0.62-0.67 (2H, m), 1.51-1.58 (4H, m), 2.53-2.57 (1H, m), 3.71 (8H, s), 6.48 (1H, d), 6.75 (1H, s), 7.51 (2H, d), 8.15 (2H, d), 8.63 (1H, s), 14.02 (1H, s)

LCMS Spectrum: m/z (ESI+) (M+H)+=424; HPLC tR=1.09 min.

### Methyl 1-[2-[4-(cyclopropylcarbamoylamino)phenyl]-6-morpholin-4-ylpyrimidin-4-yl]cyclopropane-1-carboxylate

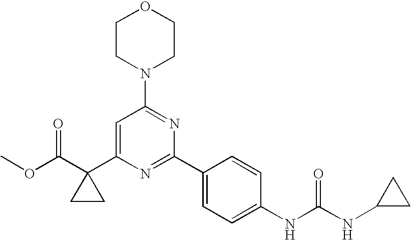

Cyclopropanamine (0.219 mL, 3.15 mmol) was added in one portion to methyl 1-[6-morpholin-4-yl-2-[4-(phenoxycarbonylamino)phenyl]pyrimidin-4-yl]cyclopropane-1-carboxylate (998 mg, 2.10 mmol) in DMF (20 mL) The resulting solution was stirred at RT for 4 hours and the reaction mixture evaporated to dryness. The crude product was purified by ion exchange chromatography using an SCX column, eluting with methanol followed by 7M ammonia in methanol, to give the desired material as a yellow solid (757 mg).

NMR Spectrum: ¹H NMR (400.13 MHz, DMSO-d₆) δ 0.39-0.43 (2H, m), 0.62-0.67 (2H, m), 1.50-1.53 (4H, m), 2.52-2.56 (1H, m), 3.64 (3H, s), 3.69 (8H, d), 6.43 (1H, d), 6.84 (1H, s), 7.47-7.49 (2H, m), 8.18 (2H, d), 8.55 (1H, s)

LCMS Spectrum: m/z (ESI+) (M+H)+=438; HPLC tR=2.10 min.

The preparation of methyl 1-[6-morpholin-4-yl-2-[4-(phenoxycarbonylamino)phenyl]pyrimidin-4-yl]cyclopropane-1-carboxylate was described earlier.

### EXAMPLE 34

### 1-[2-[4-(2-Hydroxyethylcarbamoylamino)phenyl]-6-morpholin-4-ylpyrimidin-4-yl]-N,N-dimethylcyclopropane-1-carboxamide

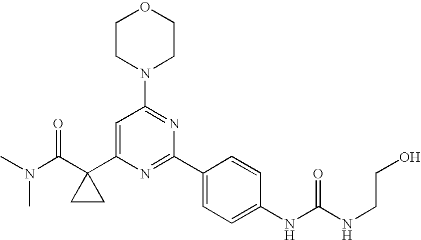

DIPEA (0.106 mL, 0.60 mmol) was added to a suspension of 1-[2-[4-(2-hydroxyethylcarbamoylamino)phenyl]-6-morpholin-4-ylpyrimidin-4-yl]cyclopropane-1-carboxylic acid (86 mg, 0.20 mmol), dimethylamine (2.0M in THF, 0.30 mL, 0.60 mmol) and HBTU (115 mg, 0.30 mmol) in DMF (3 mL). The reactions were stirred at RT for 1 hour then purified by ion exchange using a SCX-2 column, eluting with methanol followed by 7N ammonia in methanol. The isolated material was further purified by preparative HPLC, using decreasingly polar mixtures of water (containing 1% ammonia) and acetonitrile as eluents, to give the desired material as a white solid (56 mg).

NMR Spectrum: ¹H NMR (399.9 MHz, DMSO-d₆) δ 1.30-1.31 (2H, m), 1.53 (2H, d), 2.89 (3H, s), 2.94 (3H, s), 3.17-3.19 (2H, m), 3.47 (2H, d), 3.65 (4H, d), 3.70-3.71 (4H, d), 4.74 (1H, s), 6.23 (2H, m), 7.47 (2H, d), 8.18 (2H, d), 8.79 (1H, s)

LCMS Spectrum: m/z (ESI+)(M+H)+=455; HPLC tR=1.52 min.

mTOR Kinase Assay (Echo): 0.00405 μM

The following compounds were made in an analogous fashion from 1-[2-[4-(2-hydroxyethylcarbamoylamino)phenyl]-6-morpholin-4-ylpyrimidin-4-yl]cyclopropane-1-carboxylic acid and the appropriate amine.

| | | | LCMS | Retention |
|---|---|---|---|---|
| Example | Structure | NAME | MH+ | time (min) |
| 34a | 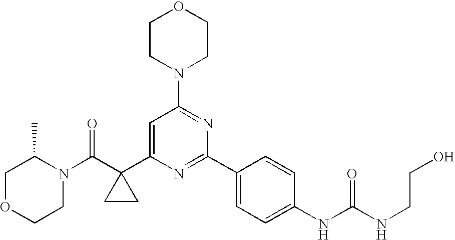 | 3-(2-hydroxyethyl)-1-[4-[4-[1-[(3S)-3-methylmorpholine-4-carbonyl]cyclopropyl]-6-morpholin-4-ylpyrimidin-2-yl]phenyl]urea | 511 | 1.59 |
| 34b | 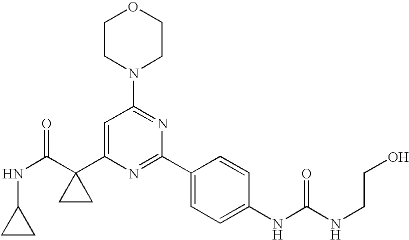 | N-cyclopropyl-1-[2-[4-(2-hydroxyethylcarbamoylamino)phe-nyl]-6-morpholin-4-ylpyrimidin-4-yl]cyclopropane-1-carboxamide | 467 | 1.56 |
| 34c | 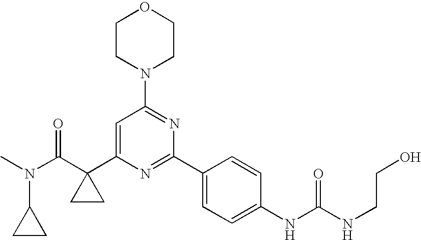 | N-cyclopropyl-1-[2-[4-(2-hydroxyethylcarbamoylamino)phe-nyl]-6-morpholin-4-ylpyrimidin-4-yl]-N-methylcyclopropane-1-carboxamide | 481 | 1.67 |
| 34d | 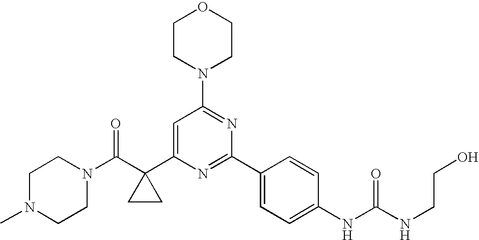 | 3-(2-hydroxyethyl)-1-[4-[4-[1-(4-methylpiperazine-1-carbonyl)cyclopropyl]-6-morpholin-4-ylpyrimidin-2-yl]phenyl]urea | 510 | 1.44 |

### EXAMPLE 34a

¹H NMR (399.9 MHz, DMSO-d₆) δ 1.22-1.25 (3H, m), 1.33 (2H, d), 1.63 (2H, d), 3.16-3.20 (1H, m), 3.17-3.19 (3H, m), 3.46 (3H, m), 3.67 (4H, s), 3.71 (4H, d), 3.71 (2H, s), 4.20 (1H, d), 4.50(1H, s), 4.74 (1H, t), 6.25 (2H, m), 7.47 (2H, d), 8.20 (2H, d), 8.79 (1H, s).

mTOR Kinase Assay (Echo): 0.0387 μM

### EXAMPLE 34b

¹H NMR (399.9 MHz, DMSO-d₆) δ 0.44-0.46 (2H, m), 0.62-0.65 (2H, m), 1.32-1.37 (4H, m), 2.73 (1H, d), 3.18 (2H, d), 3.46 (2H, t), 3.68 (4H, d), 3.71-3.71 (4H, d), 4.74 (1H, t), 6.25 (1H, s), 6.46 (1H, s), 7.47-7.50 (2H, m), 8.15-8.18 (2H, m), 8.35 (1H, s), 8.80 (1H, s).

mTOR Kinase Assay (Echo): 0.0109 μM

### EXAMPLE 34c

¹H NMR (399.9 MHz, DMSO-d₆) δ 0.69 (4H, s), 1.50 (4H, m), 2.40 (1H, s), 2.80 (3H, m), 3.16-3.19 (2H, m), 3.47 (2H, q), 3.65 (4H, d), 3.70-3.72 (4H, m), 4.74 (1H, t), 6.25 (2H, m), 7.46-7.48 (2H, m), 8.17-8.19 (2H, m), 8.78 (1H, s).

mTOR Kinase Assay (Echo): 0.00345 μM

### EXAMPLE 34d

¹H NMR (399.9 MHz, DMSO-d₆) δ 1.30-1.33 (2H, m), 1.51-1.54 (2H, m), 2.10-2.15 (5H, m), 2.33-2.34 (1H, t), 3.18-3.19 (3H, m), 3.36 (1H, d), 3.47 (2H, q), 3.58 (3H, s), 3.66-3.71 (4H, m), 3.71 (4H, d), 4.74 (1H, t), 6.25 (1H, t), 6.27 (1H, s), 7.46-7.49 (2H, m), 8.18-8.21 (2H, m), 8.79 (1H, s).

mTOR Kinase Assay (Echo): 0.0863 μM

The preparation of 1-[2-[4-(2-hydroxyethylcarbamoylamino)phenyl]-6-morpholin-4-ylpyrimidin-4-yl]cyclopropane-1-carboxylic acid is described below:

### 1-[2-[4-(2-Hydroxyethylcarbamoylaminophenyl]-6-morpholin-4-ylpyrimidin-4-yl]cyclopropane-1-carboxylic acid

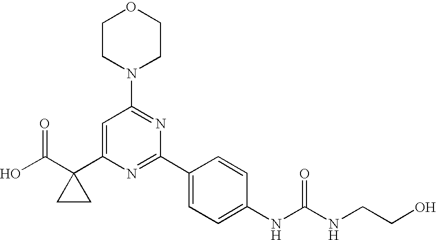

Lithium hydroxide-1-hydrate (67.2 mg, 1.60 mmol) was added in one portion to methyl 1-[2-[4-(2-hydroxyethylcarbamoylamino)phenyl]-6-morpholin-4-ylpyrimidin-4-yl]cyclopropane-1-carboxylate (707 mg, 1.60 mmol) in methanol (8 mL) and water (8 mL). The resulting suspension was stirred at 80° C. for 1 hour. The reaction mixture was evaporated to dryness, redissolved in water (25 mL), and washed with diethyl ether (25 mL). The aqueous layer was acidified with 2M hydrochloric acid, the precipitate collected by filtration, washed with diethyl ether and dried under vacuum to give the desired material as a beige solid (439 mg).

NMR Spectrum: ¹H NMR (400.13 MHz, DMSO-d₆) δ 1.51-1.58 (4H, m), 3.17 (2H, q), 3.45 (2H, t), 3.70 (8H, s), 4.77 (1H, s), 6.28 (1H, t), 6.74 (1H, s), 7.48-7.50 (2H, m), 8.14-8.16 (2H, m), 8.86 (1H, s), 14.06 (1H, s)

LCMS Spectrum: m/z (ESI+) (M+H)+=428; HPLC tR=0.92 min.

### Methyl 1-[2-[4-(2-hydroxyethylcarbamoylamino)phenyl]-6-morpholin-4-ylpyrimidin-4-yl]cyclopropane-1-carboxylate

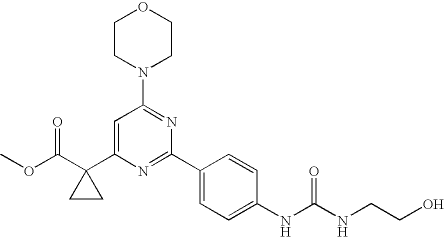

2-Aminoethanol (0.127 mL, 2.10 mmol) was added in one portion to methyl 1-[6-morpholin-4-yl-2-[4-(phenoxycarbonylamino)phenyl]pyrimidin-4-yl]cyclopropane-1-carboxylate (998 mg, 2.10 mmol) in DMF (20 mL) The resulting solution was stirred at RT for 16 hours. The reaction mixture was evaporated to dryness and the crude product purified by ion exchange chromatography using an SCX column, eluting with methanol followed by 7N ammonia in methanol, to give the desired material as a yellow solid (767 mg).

NMR Spectrum: ¹H NMR (400.13 MHz, DMSO-d₆) δ 1.50-1.52 (4H, m), 3.17 (2H, q), 3.45 (2H, q), 3.64 (3H, s), 3.70 (8H, s), 4.77 (1H, t), 6.25 (1H, t), 6.83 (1H, s), 7.45-7.47 (2H, m), 8.16-8.19 (2H, m), 8.81 (1H, s)

LCMS Spectrum. m/z (ESI+) (M+H)+=442; HPLC tR=1.71 min.

The preparation of methyl 1-[6-morpholin-4-yl-2-[4-(phenoxycarbonylamino)phenyl]pyrimidin-4-yl]cyclopropane-1-carboxylate was described earlier.

### EXAMPLE 35

### 3-Methyl-1-[4-[4-[(3S)-3-methylmorpholin-4-yl]-6-(1-methylsulfonylcyclopropyl)pyrimidin-2-yl]phenyl]thiourea

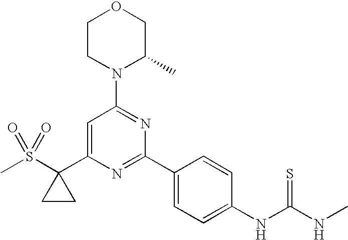

To a solution of 4-[4-[(3S)-3-methylmorpholin-4-yl]-6-(1-methylsulfonylcyclopropyl)pyrimidin-2-yl]aniline (60 mg, 0.16 mmol) in DCM (2 mL) and THF (1 mL) was added a solution of di(imidazol-1-yl)methanethione (37 mg, 0.21 mmol) in DCM (1 mL) and the resulting solution stirred at RT for 3 hours. Methylamine (2M in THF, 0.4 mL, 0.80 mmol) was added followed by triethylamine (0.022 mL, 0.16 mmol) and the solution stirred for 1 hour at RT. The solvent was evaporated and the crude product purified by preparative HPLC, using decreasingly polar mixtures of water (containing 1% NH3) and MeCN as eluents, to give the desired material as a white solid (19 mg).

NMR Spectrum: ¹H NMR (400.13 MHz, DMSO-d₆) δ 1.24 (3H, d), 1.55-1.58 (2H, m), 1.66-1.69 (2H, m), 2.95 (3H, d), 3.16 (3H, s), 3.21-3.26 (1H, m), 3.45-3.52 (1H, m), 3.61-3.65 (1H, m), 3.76 (1H, d), 3.95-3.99 (1H, m), 4.20-4.23 (1H, m), 4.55-4.63 (1H, m), 6.80 (1H, s), 7.55 (2H, d), 7.84 (1H, s), 8.26 (2H, d), 9.73 (1H, s)

LCMS Spectrum: m/z (ESI+)(M+H)+=462; HPLC tR=1.86 min.

mTOR Kinase Assay (Echo): 0.00531 μM

The compounds below were prepared in an analogous fashion from 4-[4-[(3S)-3-methylmorpholin-4-yl]-6-(1-methylsulfonylcyclopropyl)pyrimidin-2-yl]aniline using the appropriate amine.

| | | | LCMS | Retention |
|---|---|---|---|---|
| Example | Structure | NAME | MH+ | time (min) |
| 35a | 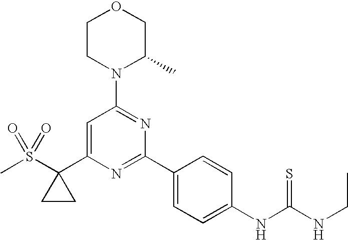 | 3-ethyl-1-[4-[4-[(3S)-3-methylmorpholin-4-yl]-6-(1-methylsulfonylcyclopropyl)pyrimidin-2-yl]phenyl]thiourea | 476 | 1.94 |
| 35b | 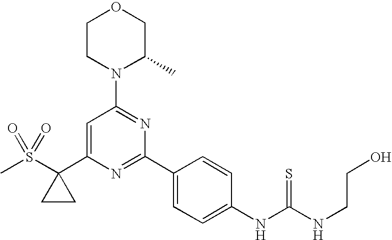 | 3-(2-hydroxyethyl)-1-[4-[4-[(3S)-3-methylmorpholin-4-yl]-6-(1-methylsulfonylcyclopropyl)pyrimidin-2-yl]phenyl]thiourea | 492 | 1.68 |
| 35c | 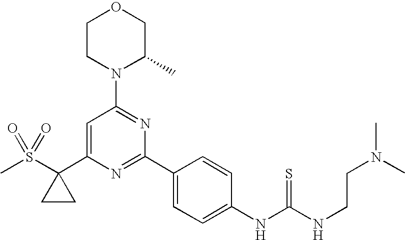 | 3-(2-dimethylaminoethyl)-1-[4-[4-[(3S)-3-methylmorpholin-4-yl]-6-(1-methylsulfonylcyclopropyl)pyrimidin-2-yl]phenyl]thiourea | 519 | 1.99 |
| 35d | 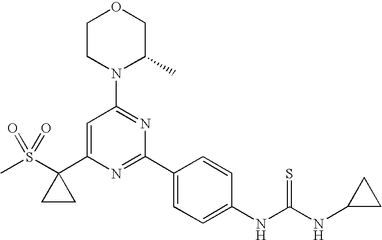 | 3-cyclopropyl-1-[4-[4-[(3S)-3-methylmorpholin-4-yl]-6-(1-methylsulfonylcyclopropyl)pyrimidin-2-yl]phenyl]thiourea | 488 | 1.89 |

### EXAMPLE b 35a

¹H NMR (400.13 MHz, DMSO-d₆) δ 1.14 (3H, t), 1.24 (3H, d), 1.55-1.58 (2H, m), 1.66-1.69 (2H, m), 3.17-3.25 (1H, m), 3.29 (3H, s), 3.45-3.51 (3H, m), 3.61-3.65 (1H, m), 3.76 (1H, d), 3.95-3.99 (1H, m), 4.20-4.23 (1H, m), 4.55-4.62 (1H, m), 6.80 (1H, s), 7.57 (2H, d), 7.88 (1H, s), 8.25 (2H, d), 9.63 (1H, s).

mTOR Kinase Assay (Echo): 0.00552 μM

### EXAMPLE 35b

¹H NMR (400.13 MHz, DMSO-d₆) δ 1.24 (3H, d), 1.55-1.58 (2H, m), 1.66-1.69 (2H, m), 3.17-3.25 (1H, m), 3.29 (3H, s), 3.45-3.52 (1H, m), 3.57 (4H, s), 3.61-3.65 (1H, m), 3.76 (1H, d), 3.95-3.99 (1H, m), 4.20-4.23 (1H, m), 4.55-4.62 (1H, m), 4.77-4.85 (1H, m), 6.80 (1H, s), 7.63 (2H, d), 7.86 (1H, s), 8.26 (2H, d), 9.81 (1H, s).

mTOR Kinase Assay (Echo): 0.000577 μM

### EXAMPLE 35c

¹H NMR (400.13 MHz, DMSO-d₆) δ 1.24 (3H, d), 1.55-1.58 (2H, m), 1.66-1.69 (2H, m), 2.20 (6H, s), 2.45 (2H, t), 3.18-3.25 (1H, m), 3.26 (3H, s), 3.45-3.52 (1H, m), 3.56 (2H, s), 3.61-3.65 (1H, m), 3.76 (1H, d), 3.95-3.99 (1H, m), 4.21 (1H, d), 4.56-4.61 (1H, m), 6.80 (1H, s), 7.65 (2H, d), 7.77 (1H, s), 8.25 (2H, d), 9.90 (1H, s).

mTOR Kinase Assay (Echo): 0.108 μM

### EXAMPLE 35d

¹H NMR (400.13 MHz, DMSO-d₆) δ 0.58-0.62 (2H, m), 0.74-0.79 (2H, m), 1.24 (3H, d), 1.55-1.59 (2H, m), 1.67-1.69 (2H, m), 2.89-2.97 (1H, m), 3.17-3.27 (1H, m), 3.30 (3H, s), 3.46-3.52 (1H, m), 3.62-3.66 (1H, m), 3.77 (1H, d), 3.96-3.99 (1H, m), 4.21-4.24 (1H, m), 4.60 (1H, s), 6.81 (1H, s), 7.62 (2H, d), 8.14 (1H, s), 8.25 (2H, d), 9.51 (1H, s).

mTOR Kinase Assay (Echo): 0.00411 μM

The preparation of 4-[4-[(3S)-3-methylmorpholin-4-yl]-6-(1-methylsulfonylcyclopropyl)pyrimidin-2-yl]aniline was described earlier.

### EXAMPLE 36

The following compounds were prepared according to the following general procedure:

The appropriate phenyl carbamate and an excess of both triethylamine and the appropriate amine were dissolved in either DMF, NMP or DMA and stirred at 50° C.-70° C. for between 2-18 hours (unless otherwise specified). The materials were purified by preparative HPLC except where specified.

| | | | | Reten- |
|---|---|---|---|---|
| | | | | tion |
| | | | LCMS | time |
| Example | Structure | NAME | MH+ | (min) |
| 36a | 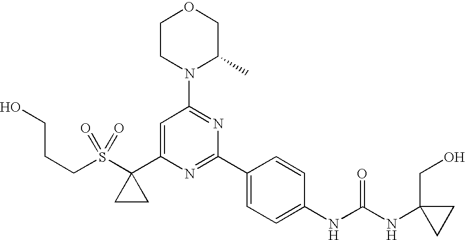 | 3-[1-(hydroxymethyl)cyclopro-pyl]-1-[4-[4-[1-(3-hydroxypropylsulfonyl)cyclopropyl]-6-[(3S)-3-methylmorpholin-4-yl]pyrimidin-2-yl]phenyl]urea | 546 | 1.67 |
| 36b | 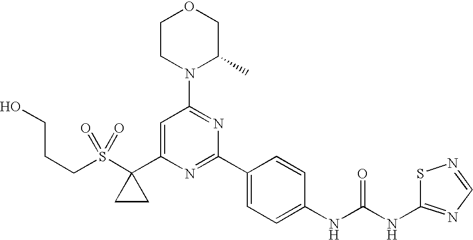 | 1-[4-[4-[1-(3-hydroxypropylsulfonyl)cyclopro-pyl]-6-[(3S)-3-methylmorpholin-4-yl]pyrimidin-2-yl]phenyl]-3-(1,2,4-thiadiazol-5-yl)urea | 560 | 1.41 |
| 36c | 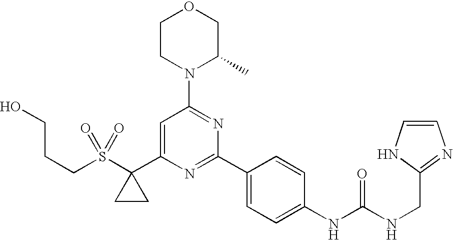 | 1-[4-[4-[1-(3-hydroxypropylsulfonyl)cyclopro-pyl]-6-[(3S)-3-methylmorpholin-4-yl]pyrimidin-2-yl]phenyl]-3-(1H-imidazol-2-ylmethyl)urea | 556 | 1.65 |
| 36d | 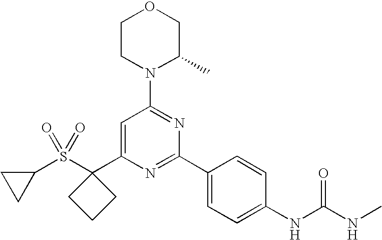 | 1-[4-[4-(1-cyclopropylsulfonylcyclobutyl)-6-[(3S)-3-methylmorpholin-4-yl]pyrimidin-2-yl]phenyl]-3-methylurea | 486 | 1.92 |
| 36e | 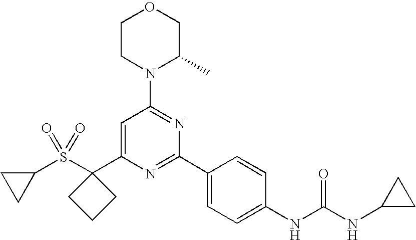 | 3-cyclopropyl-1-[4-[4-(1-cyclopropylsulfonylcyclobutyl)-6-[(3S)-3-methylmorpholin-4-yl]pyrimidin-2-yl]phenyl]urea | 512 | 2.16 |
| 36f | 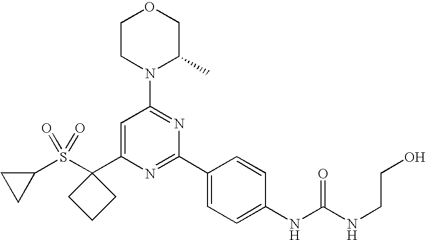 | 1-[4-[4-(1-cyclopropylsulfonylcyclobutyl)-6-[(3S)-3-methylmorpholin-4-yl]pyrimidin-2-yl]phenyl]-3-(2-hydroxyethyl)urea | 516 | 1.84 |
| 36g | 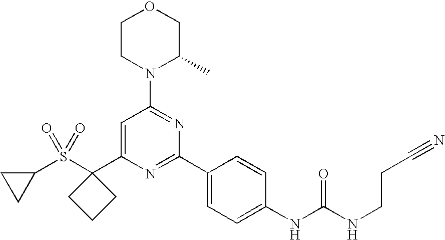 | 3-(2-cyanoethyl)-1-[4-[4-(1-cyclopropylsulfonylcyclobutyl)-6-[(3S)-3-methylmorpholin-4-yl]pyrimidin-2-yl]phenyl]urea | 525 | 2.07 |
| 36h | 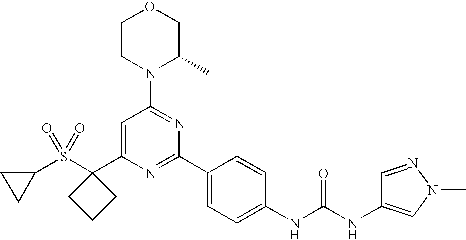 | 1-[4-[4-(1-cyclopropylsulfonylcyclobutyl)-6-[(3S)-3-methylmorpholin-4-yl]pyrimidin-2-yl]phenyl]-3-(1-methylpyrazol-4-yl)urea | 552 | 2.04 |
| 36i | 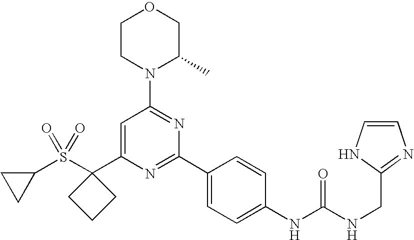 | 1-[4-[4-(1-cyclopropylsulfonylcyclobutyl)-6-[(3S)-3-methylmorpholin-4-yl]pyrimidin-2-yl]phenyl]-3-(1H-imidazol-2-ylmethyl)urea | 552 | 1.99 |
| 36j | 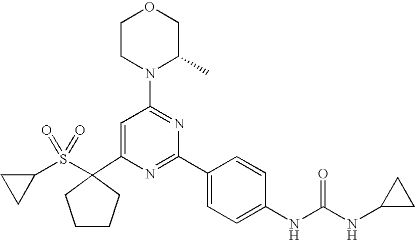 | 3-cyclopropyl-1-[4-[4-(1-cyclopropylsulfonylcyclopentyl)-6-[(3S)-3-methylmorpholin-4-yl]pyrimidin-2-yl]phenyl]urea | 526 | 2.34 |
| 36k | 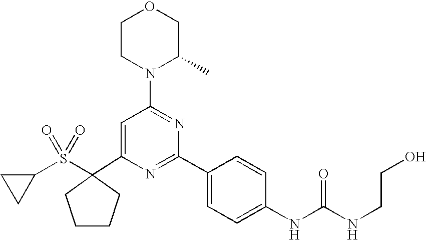 | 1-[4-[4-(1-cyclopropylsulfonylcyclopentyl)-6-[(3S)-3-methylmorpholin-4-yl]pyrimidin-2-yl]phenyl]-3-(2-hydroxyethyl)urea | 530 | 1.97 |
| 36l | 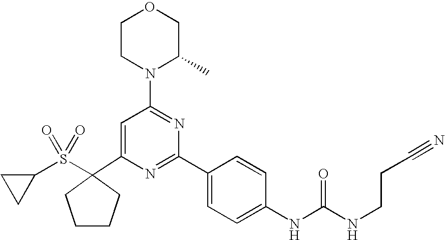 | 3-(2-cyanoethyl)-1-[4-[4-(1-cyclopropylsulfonylcyclopentyl)-6-[(3S)-3-methylmorpholin-4-yl]pyrimidin-2-yl]phenyl]urea | 539 | 2.19 |
| 36m | 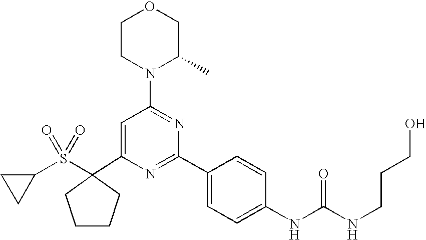 | 1-[4-[4-(1-cyclopropylsulfonylcyclopentyl)-6-[(3S)-3-methylmorpholin-4-yl]pyrimidin-2-yl]phenyl]-3-(3-hydroxypropyl)urea | 544 | 1.99 |
| 36n | 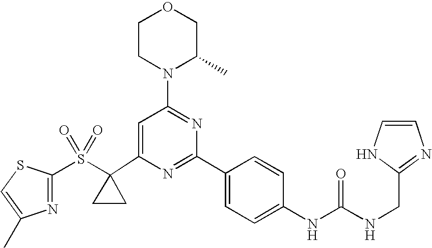 | 3-(1H-imidazol-2-ylmethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 595 | 2.07 |
| 36o | 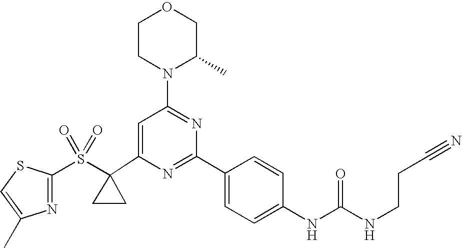 | 3-(2-cyanoethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 568 | 2.25 |
| 36p | 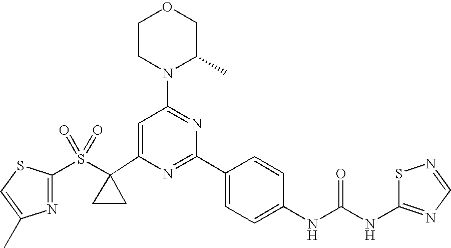 | 1-[4-[4-[(3S)-3-methylmorpholin-4-yl]-6-[1-[(4-methyl-1,3-thiazol-2-yl)sulfonyl]cyclopropyl]pyrimidin-2-yl]phenyl]-3-(1,2,4-thiadiazol-5-yl)urea | 599 | 1.81 |
| 36q | 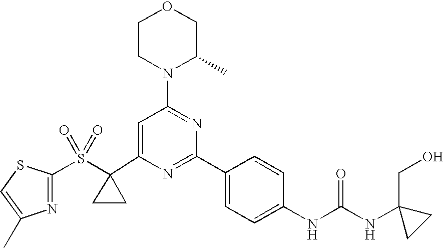 | 3-[1-(hydroxymethyl)cyclopropyl]-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 585 | 2.09 |
| 36r | 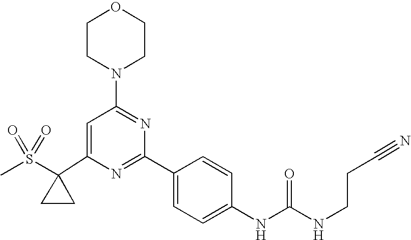 | 3-(2-cyanoethyl)-1-[4-[4-(1-methylsulfonylcyclopropyl)-6-morpholin-4-ylpyrimidin-2-yl]phenyl]urea | 471.5 | 1.66 |
| 36s* | 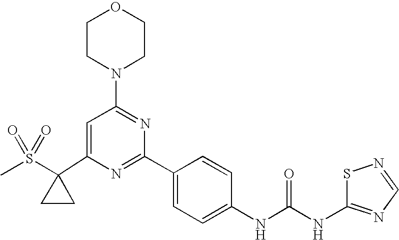 | 1-[4-[4-(1-methylsulfonylcyclopropyl)-6-morpholin-4-ylpyrimidin-2-yl]phenyl]-3-(1,2,4-thiadiazol-5-yl)urea | 502 | 1.61 |
| 36t | 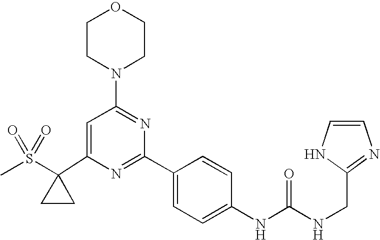 | 3-(1H-imidazol-2-ylmethyl)-1-[4-[4-(1-methylsulfonylcyclopropyl)-6-morpholin-4-ylpyrimidin-2-yl]phenyl]urea | 498.6 | 2.02 |
| 36u | 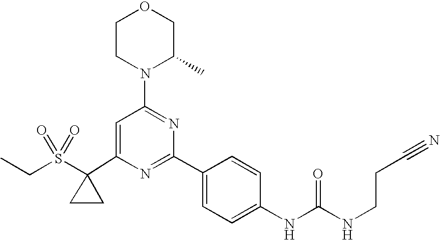 | 3-(2-cyanoethyl)-1-[4-[4-(1-ethylsulfonylcyclopropyl)-6-[(3S)-3-methylmorpholin-4-yl]pyrimidin-2-yl]phenyl]urea | 499.1 | 2.10 |
| 36v | 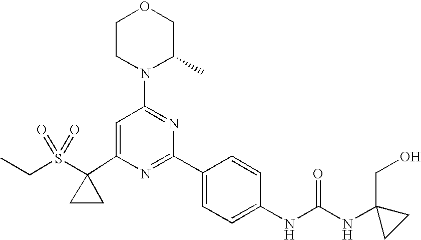 | 1-[4-[4-(1-ethylsulfonylcyclopropyl)-6-[(3S)-3-methylmorpholin-4-yl]pyrimidin-2-yl]phenyl]-3-[1-(hydroymethyl)cyclopropyl]urea | 516.1 | 1.97 |
| 36w | 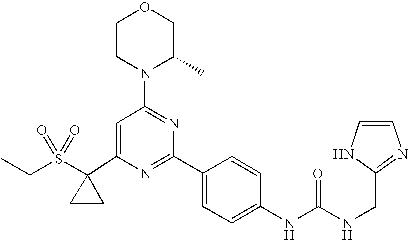 | 1-[4-[4-(1-ethylsulfonylcyclopropyl)-6-[(3S)-3-methylmorpholin-4-yl]pyrimidin-2-yl]phenyl]-3-(1H-imidazol-2-ylmethyl)urea | 526.1 | 1.94 |
| 36x | 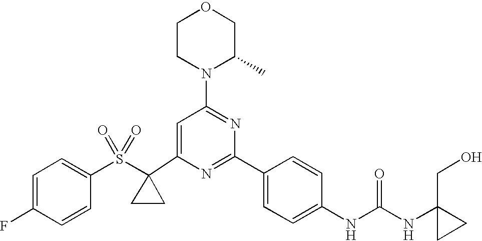 | 1-[4-[4-[1-(4-fluorophenyl)sulfonylcyclopropyl]-6-[(3S)-3-methylmorpholin-4-yl]pyrimidin-2-yl]phenyl]-3-[1-(hydroxymethyl)cyclopropyl]urea | 582 | 2.17 |
| 36y | 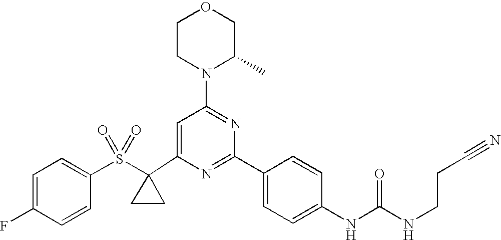 | 3-(2-cyanoethyl)-1-[4-[4-[1-(4-fluorophenyl)sulfonylcyclopropyl]-6-[(3S)-3-methylmorpholin-4-yl]pyrimidin-2-yl]phenyl]urea | 565 | 2.40 |
| 36z | 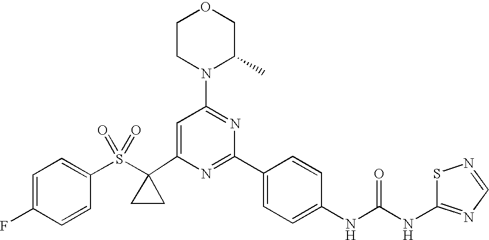 | 1-[4-[4-[1-(4-fluorophenyl)sulfonylcyclopropyl]-6-[(3S)-3-methylmorpholin-4-yl]pyrimidin-2-yl]phenyl]-3-(1,2,4-thiadiazol-5-yl)urea | 596 | 1.49 |
| 36aa | 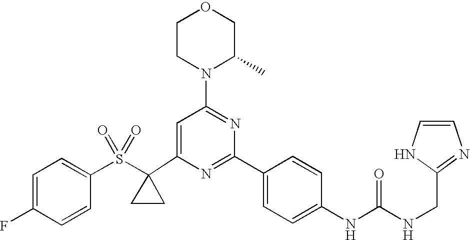 | 1-[4-[4-[1-(4-fluorophenyl)sulfonylcyclopropyl]-6-[(3S)-3-methylmorpholin-4-yl]pyrimidin-2-yl]phenyl]-3-(1H-imidazol-2-ylmethyl)urea | 592 | 2.07 |
| 36ab | 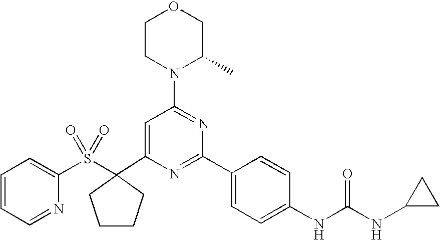 | 3-cyclopropyl-1-[4-[4-[(3S)-3-methylmorpholin-4-yl]-6-(1-pyridin-2-ylsulfonylcyclopentyl)pyrimidin-2-yl]phenyl]urea | 563 | 2.38 |
| 36ac | 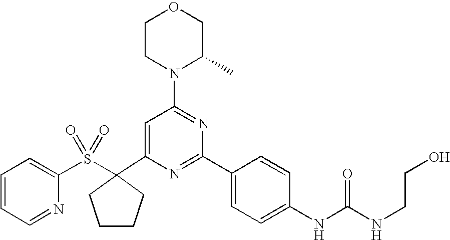 | 3-(2-hydroxyethyl)-1-[4-[4-[(3S)-3-methylmorpholin-4-yl]-6-(1-pyridin-2-ylsulfonylcyclopentyl)pyrimidin-2-yl]phenyl]urea | 567 | 2.01 |
| 36ad | 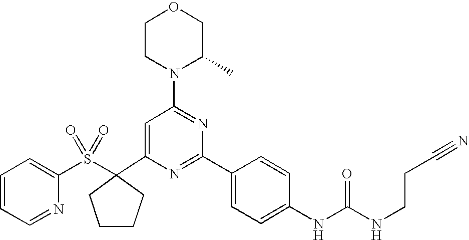 | 3-(2-cyanoethyl)-1-[4-[4-[(3S)-3-methylmorpholin-4-yl]-6-(1-pyridin-2-ylsulfonylcyclopentyl)pyrimidin-2-yl]phenyl]urea | 576 | 2.28 |
| 36ae | 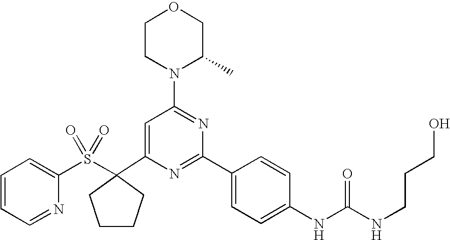 | 3-(3-hydroxypropyl)-1-[4-[4-[(3S)-3-methylmorpholin-4-yl]-6-(1-pyridin-2-ylsulfonylcyclopentyl)pyrimidin-2-yl]phenyl]urea | 581 | 2.05 |
| 36af | 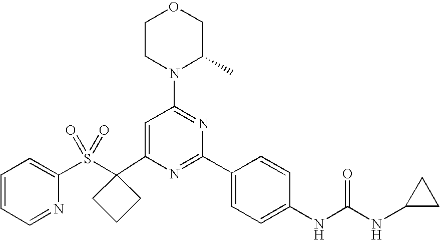 | 3-cyclopropyl-1-[4-[4-[(3S)-3-methylmorpholin-4-yl]-6-(1-pyridin-2-ylsulfonylcyclobutyl)pyrimidin-2-yl]phenyl]urea | 549 | 2.25 |
| 36ag | 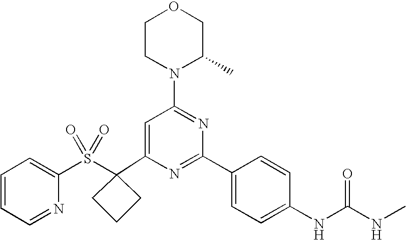 | 3-methyl-1-[4-[4-[(3S)-3-methylmorpholin-4-yl]-6-(1-pyridin-2-ylsulfonylcyclobutyl)pyrimidin-2-yl]phenyl]urea | 523 | 2.08 |
| 36ah | 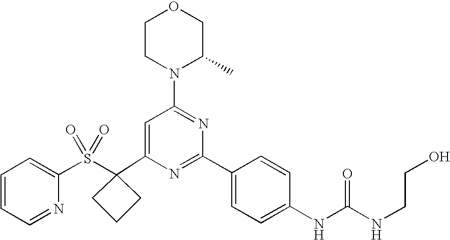 | 3-(2-hydroxyethyl)-1-[4-[4-[(3S)-3-methylmorpholin-4-yl]-6-(1-pyridin-2-ylsulfonylcyclobutyl)pyrimidin-2-yl]phenyl]urea | 553 | 1.89 |
| 36ai | 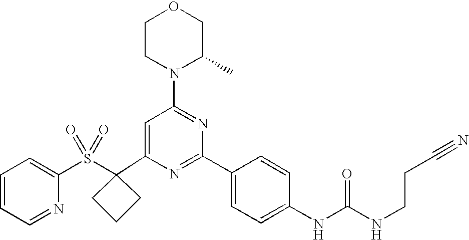 | 3-(2-cyanoethyl)-1-[4-[4-[(3S)-3-methylmorpholin-4-yl]-6-(1-pyridin-2-ylsulfonylcyclobutyl)pyrimidin-2-yl)phenyl]urea | 562 | 2.15 |
| 36aj | 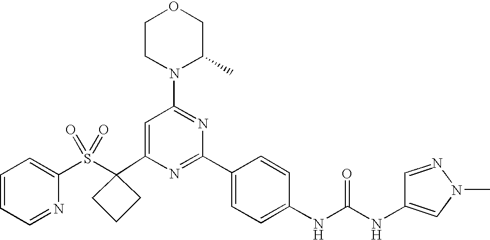 | 1-[4-[4-[(3S)-3-methylmorpholin-4-yl]-6-(1-pyridin-2-ylsulfonylcyclobutyl)pyrimidin-2-yl]phenyl]-3-(1-methylpyrazol-4-yl)urea | 589 | 2.11 |
| 36ak | 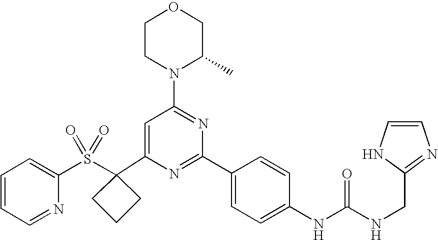 | 3-(1H-imidazol-2-ylmethyl)-1-[4-[4-[(3S)-3-methylmorpholin-4-yl]-6-(1-pyridin-2-ylsulfonylcyclobutyl)pyrimidin-2-yl]phenyl]urea | 589 | 2.34 |
| 36al** | 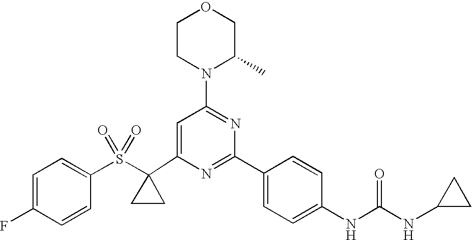 | 3-cyclopropyl-1-[4-[4-[1-(4-fluorophenyl)sulfonylcyclopropyl]-6-morpholin-4-ylpyrimidin-2-yl]phenyl]urea | 538 | 2.36 |
| 36am** | 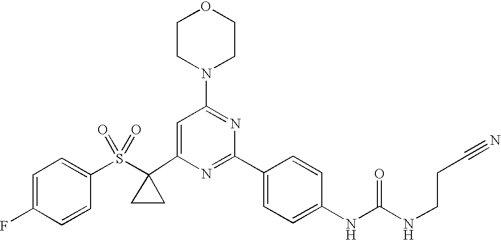 | 3-(2-cyanoethyl)-1-[4-[4-[1-(4-fluorophenyl)sulfonylcyclopropyl]-6-morpholin-4-ylpyrimidin-2-yl]phenyl]urea | 551 | 2.27 |
| 36an | 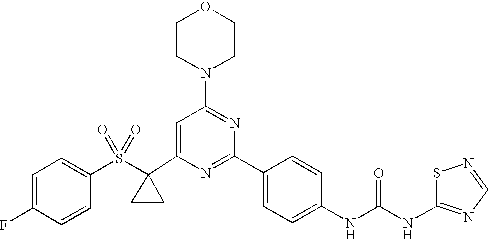 | 1-[4-[4-[1-(4-fluorophenyl)sulfonylcyclopropyl]-6-morpholin-4-ylpyrimidin-2-yl]phenyl]-3-(1,2,4-thiadiazol-5-yl)urea | 542 | 1.41 |
| 36ao | 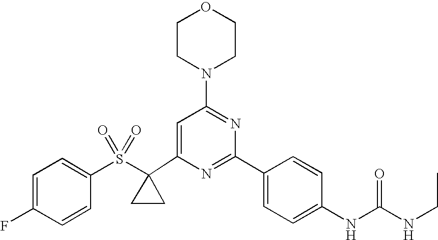 | 1-ethyl-3-[4-[4-[1-(4-fluorophenyl)sulfonylcyclopropyl]-6-morpholin-4-ylpyrimidin-2-yl]phenyl]urea | 526 | 2.14 |
| 36ap | 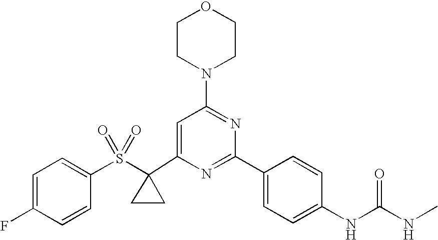 | 3-[4-[4-[1-(4-fluorophenyl)sulfonylcyclopropyl]-6-morpholin-4-ylpyrimidin-2-yl]phenyl]-1-methylurea | 512 | 1.99 |
| 36aq | 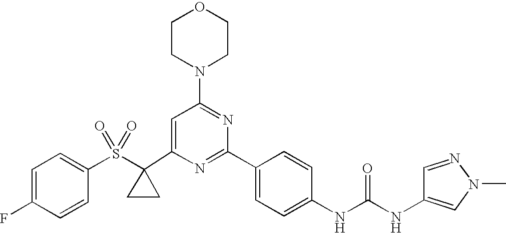 | 3-[4-[4-[1-(4-fluorophenyl)sulfonylcyclopropyl]-6-morpholin-4-ylpyrimidin-2-yl]phenyl]-1-(1-methylpyrazol-4-yl)urea | 578 | 2.04 |
| 36ar | 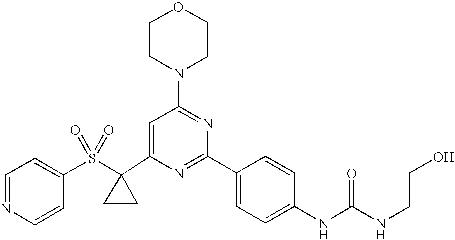 | 3-(2-hydroxyethyl)-1-[4-[4-morpholin-4-yl-6-(1-pyridin-4-ylsulfonylcyclopropyl)pyrimidin-2-yl]phenyl]urea | 525 | 1.66 |
| 36as | 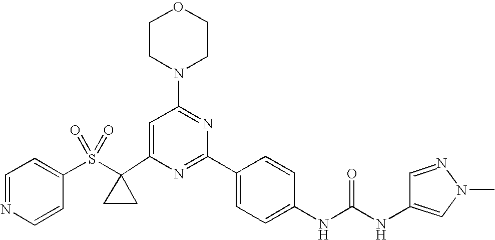 | 1-(1-methylpyrazol-4-yl)-3-[4-[4-morpholin-4-yl-6-(1-pyridin-4-ylsulfonylcyclopropyl)pyrimidin-2-yl]phenyl]urea | 561 | 1.81 |
| 36at | 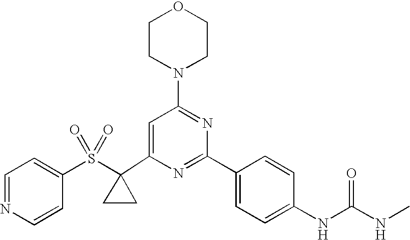 | 1-methyl-3-[4-[4-morpholin-4-yl-6-(1-pyridin-4-ylsulfonylcyclopropyl)pyrimidin-2-yl]phenyl]urea | 495 | 1.81 |
| 36au | 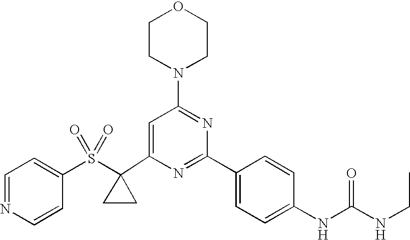 | 1-ethyl-3-[4-[4-morpholin-4-yl-6-(1-pyridin-4-ylsulfonylcyclopropyl)pyrimidin-2-yl]phenyl]urea | 509 | 1.99 |
| 36av | 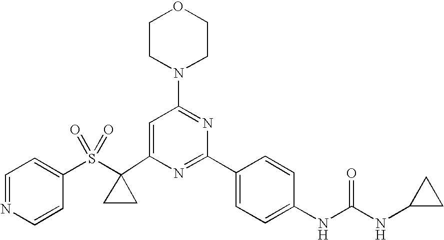 | 3-cyclopropyl-1-[4-[4-morpholin-4-yl-6-(1-pyridin-4-ylsulfonylcyclopropyl)pyrimidin-2-yl]phenyl]urea | 521 | 1.95 |
| 36aw | 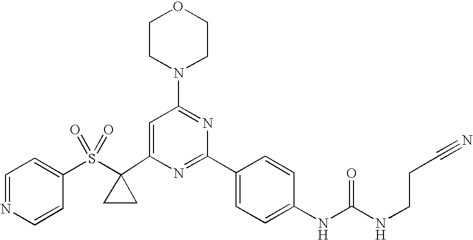 | 3-(2-cyanoethyl)-1-[4-[4-morpholin-4-yl-6-(1-pyridin-4-ylsulfonylcyclopropyl)pyrimidin-2-yl]phenyl]urea | 534 | 1.89 |
| 36ax | 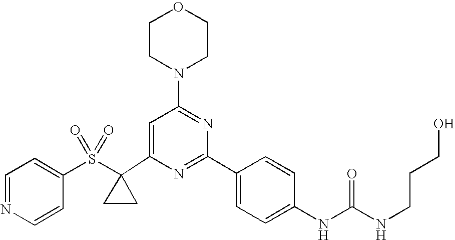 | 3-(3-hydroxypropyl)-1-[4-[4-morpholin-4-yl-6-(1-pyridin-4-ylsulfonylcyclopropyl)pyrimidin-2-yl]phenyl]urea | 539 | 1.72 |
| 36ay | 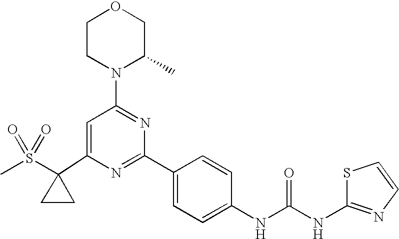 | 1-[4-[4-[(3S)-3-methylmorpholin-4-yl]-6-(1-methylsulfonylcyclopropyl)pyri-midin-2-yl]phenyl]-3-(1,3-thiazol-2-yl)urea | 515 | 2.19 |
| 36az | 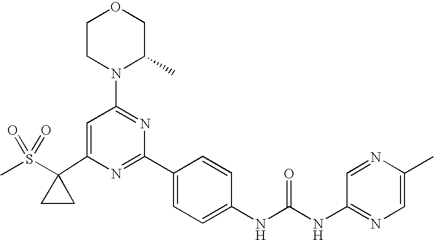 | 3-[4-[4-[(3S)-3-methylmorpholin-4-yl]-6-(1-methylsulfonylcyclopropyl)pyri-midin-2-yl]phenyl]-1-(5-methylpyrazin-2-yl)urea | 524 | 2.35 |
| 36ba | 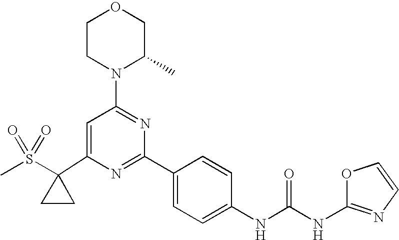 | 1-[4-[4-[(3S)-3-methylmorpholin-4-yl]-6-(1-methylsulfonylcyclopropyl)pyri-midin-2-yl]phenyl]-3-(1,3-oxazol-2-yl)urea | 499 | 1.99 |
| 36bb | 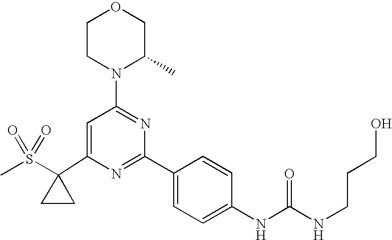 | 3-(3-hydroxypropyl)-1-[4-[4-[(3S)-3-methylmorpholin-4-yl]-6-(1-methylsulfonylcyclopropyl)pyri-midin-2-yl]phenyl]urea | 490 | 1.75 |
| 36bc | 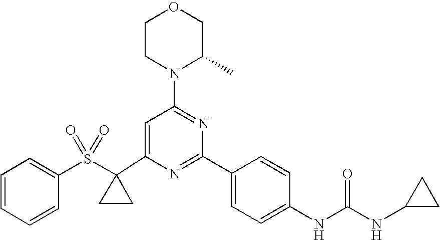 | 1-[4-[4-[1-(benzenesulfonyl)cyclopropyl]-6-[(3S)-3-methylmorpholin-4-yl]pyrimidin-2-yl]phenyl]-3-cyclopropylurea | 534 | 2.39 |
| 36bd | 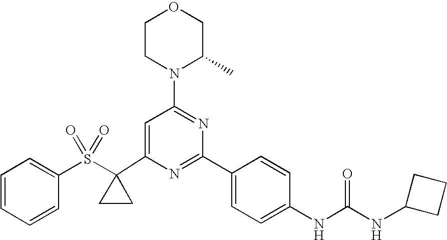 | 1-[4-[4-[1-(benzenesulfonyl)cyclopropyl]-6-[(3S)-3-methylmorpholin-4-yl]pyrimidin-2-yl]phenyl]-3-cyclobutylurea | 548 | 2.61 |
| 36be | 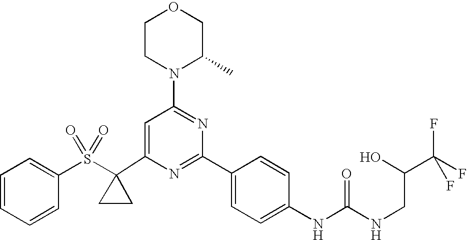 | 1-[4-[4-[1-(benzenesulfonyl)cyclopropyl]-6-[(3S)-3-methylmorpholin-4-yl]pyrimidin-2-yl]phenyl]-3-(3,3,3-trifluoro-2-hydroxypropyl)urea | 606 | 2.45 |
| 36bf | 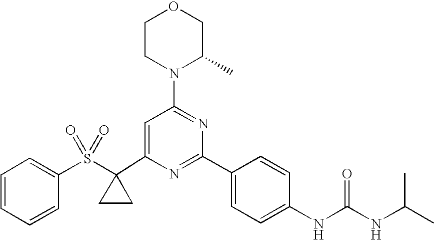 | 1-[4-[4-[1-(benzenesulfonyl)cyclopropyl]-6-[(3S)-3-methylmorpholin-4-yl]pyrimidin-2-yl]phenyl]-3-propan-2-ylurea | 536 | 2.51 |
| 36bg | 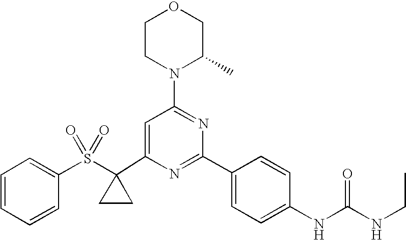 | 3-[4-[4-[1-(benzenesulfonyl)cyclopropyl]-6-[(3S)-3-methylmorpholin-4-yl]pyrimidin-2-yl]phenyl]-1-ethylurea | 522 | 2.32 |
| 36bh | 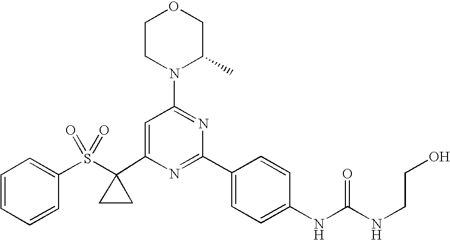 | 1-[4-[4-[1-(benzenesulfonyl)cyclopropyl]-6-[(3S)-3-methylmorpholin-4-yl]pyrimidin-2-yl]phenyl]-3-(2-hydroxyethyl)urea | 538 | 1.98 |
| 36bi | 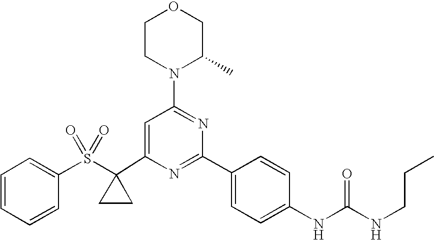 | 3-[4-[4-[1-(benzenesulfonyl)cyclopropyl]-6-[(3S)-3-methylmorpholin-4-yl]pyrimidin-2-yl]phenyl]-1-propylurea | 536 | 2.49 |
| 36bj | 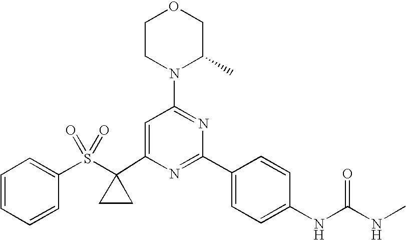 | 1-[4-[4-[1-(benzenesulfonyl)cyclopropyl]-6-[(3S)-3-methylmorpholin-4-yl]pyrimidin-2-yl]phenyl]-3-methylurea | 508 | 2.16 |
| 36bk | 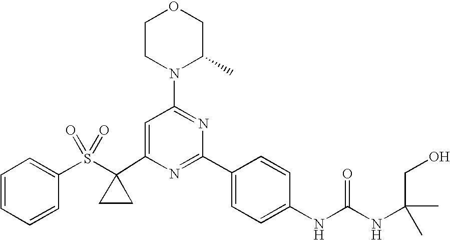 | 1-[4-[4-[1-(benzenesulfonyl)cyclopropyl]-6-[(3S)-3-methylmorpholin-4-yl]pyrimidin-2-yl]phenyl]-3-(1-hydroxy-2-methylpropan-2-yl)urea | 566 | 2.28 |
| 36bl | 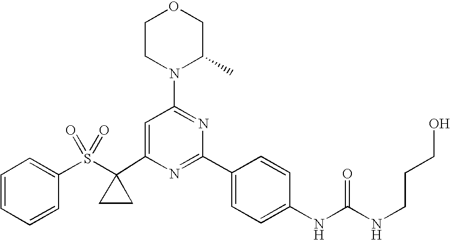 | 1-[4-[4-[1-(benzenesulfonyl)cyclopropyl]-6-[(3S)-3-methylmorpholin-4-yl]pyrimidin-2-yl]phenyl]-3-(3-hydroxypropyl)urea | 552 | 2.03 |
| 36bm | 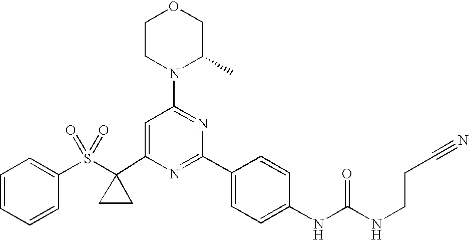 | 1-[4-[4-[1-(benzenesulfonyl)cyclopropyl]-6-[(3S)-3-methylmorpholin-4-yl]pyrimidin-2-yl]phenyl]-3-(2-cyanoethyl)urea | 547 | 2.24 |
| 36bn | 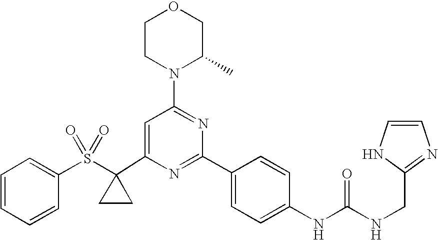 | 1-[4-[4-[1-(benzenesulfonyl)cyclopropyl]-6-[(3S)-3-methylmorpholin-4-yl]pyrimidin-2-yl]phenyl]-3-(1H-imidazol-2-ylmethyl)urea | 574 | 2.12 |
| 36bo | 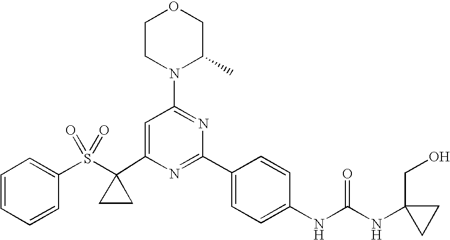 | 1-[4-[4-[1-(benzenesulfonyl)cyclopropyl]-6-[(3S)-3-methylmorpholin-4-yl]pyrimidin-2-yl]phenyl]-3-[1-(hydroxymethyl)cyclopropyl]urea | 564 | 2.11 |
| 36bp | 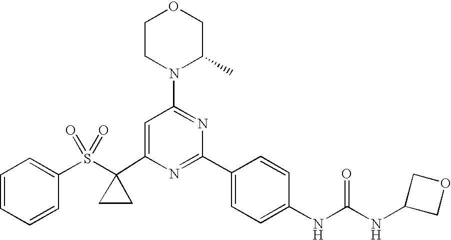 | 1-[4-[4-[1-(benzenesulfonyl)cyclopropyl]-6-[(3S)-3-methylmorpholin-4-yl]pyrimidin-2-yl]phenyl]-3-(oxetan-3-yl)urea | 550 | 2.13 |
| 36bq | 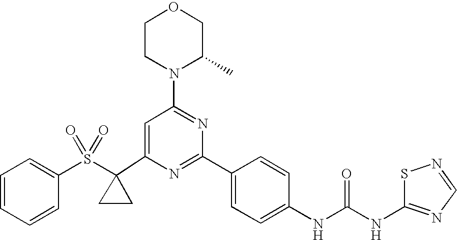 | 1-[4-[4-[1-(benzenesulfonyl)cyclopropyl]-6-[(3S)-3-methylmorpholin-4-yl]pyrimidin-2-yl]phenyl]-3-(1,2,4-thiadiazol-5-yl)urea | 578 | 1.35 |
| 36br | 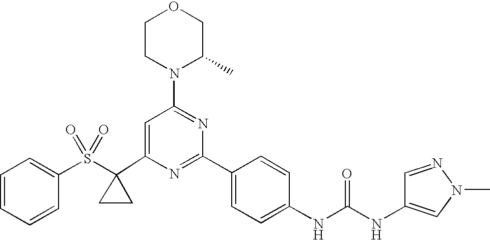 | 1-[4-[4-[1-(benzenesulfonyl)cyclopropyl]-6-[(3S)-3-methylmorpholin-4-yl]pyrimidin-2-yl]phenyl]-3-(1-methylpyrazol-4-yl)urea | 574 | 2.21 |
| 36bs | 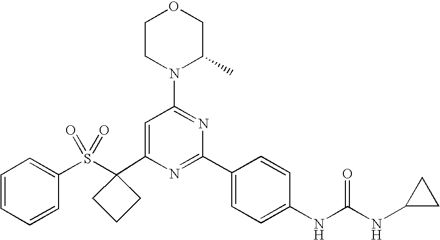 | 1-[4-[4-[1-(benzenesulfonyl)cyclobutyl]-6-[(3S)-3-methylmorpholin-4-yl]pyrimidin-2-yl]phenyl]-3-cyclopropylurea | 548 | 2.53 |
| 36bt | 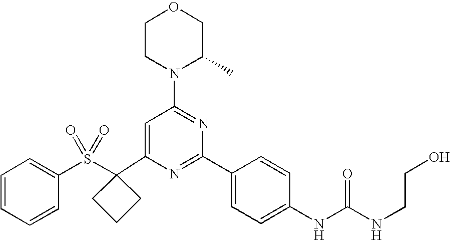 | 1-[4-[4-[1-(benzenesulfonyl)cyclobutyl]-6-[(3S)-3-methylmorpholin-4-yl]pyrimidin-2-yl]phenyl]-3-(2-hydroxyethyl)urea | 552 | 2.14 |
| 36bu | 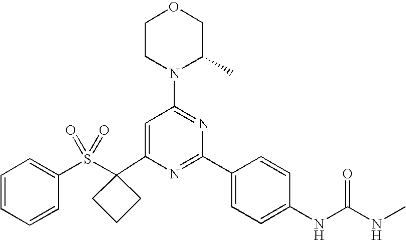 | 1-[4-[4-[1-(benzenesulfonyl)cyclobutyl]-6-[(3S)-3-methylmorpholin-4-yl]pyrimidin-2-yl]phenyl]-3-methylurea | 522 | 2.35 |
| 36bv | 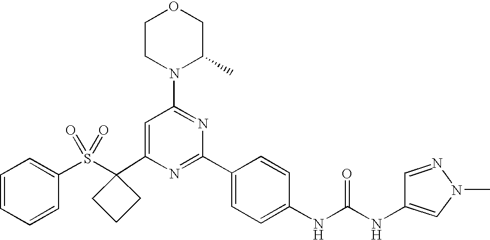 | 1-[4-[4-[1-(benzenesulfonyl)cyclobutyl]-6-[(3S)-3-methylmorpholin-4-yl]pyrimidin-2-yl]phenyl]-3-(1-methylpyrazol-4-yl)urea | 588 | 2.37 |
| 36bw | 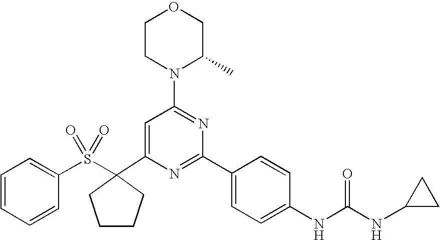 | 1-[4-[4-[1-(benzenesulfonyl)cyclopentyl]-6-[(3S)-3-methylmorpholin-4-yl]pyrimidin-2-yl]phenyl]-3-cyclopropylurea | 562 | 2.75 |
| 36bx | 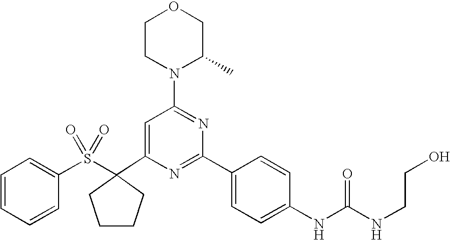 | 1-[4-[4-[1-(benzenesulfonyl)cyclopentyl]-6-[(3S)-3-methylmorpholin-4-yl]pyrimidin-2-yl]phenyl]-3-(2-hydroxyethyl)urea | 566 | 2.30 |
| 36by | 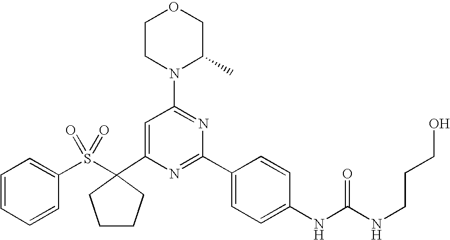 | 1-[4-[4-[1-(benzenesulfonyl)cyclopentyl]-6-[(3S)-3-methylmorpholin-4-yl]pyrimidin-2-yl]phenyl]-3-(3-hydroxypropyl)urea | 580 | 2.31 |
| 36bz | 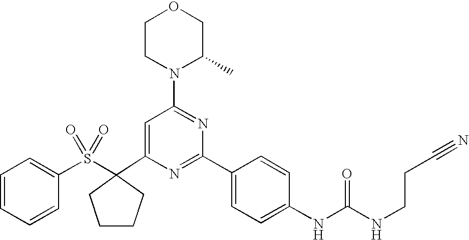 | 1-[4-[4-[1-(benzenesulfonyl)cyclopentyl]-6-[(3S)-3-methylmorpholin-4-yl]pyrimidin-2-yl]phenyl]-3-(2-cyanoethyl)urea | 575 | 2.55 |
| 36ca*** | 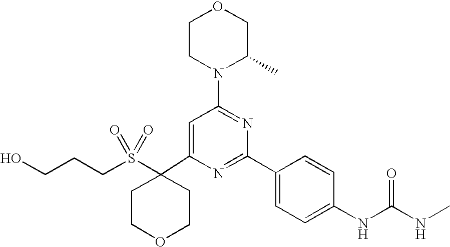 | 1-[4-[4-[4-(3-hydroxypropylsulfonyl)oxan-4-yl]-6-[(3S)-3-methylmorpholin-4-yl]pyrimidin-2-yl]phenyl]-3-methylurea | 534 | 1.56 |
| 36cb*** | 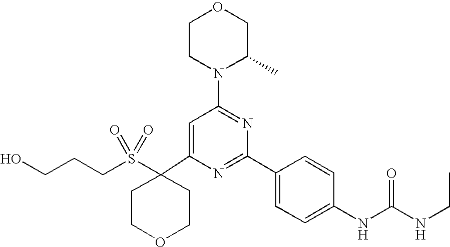 | 1-ethyl-3-[4-[4-[4-(3-hydroxypropylsulfonyl)oxan-4-yl]-6-[(3S)-3-methylmorpholin-4-yl]pyrimidin-2-yl]phenyl]urea | 548 | 1.67 |
| 36cc*** | 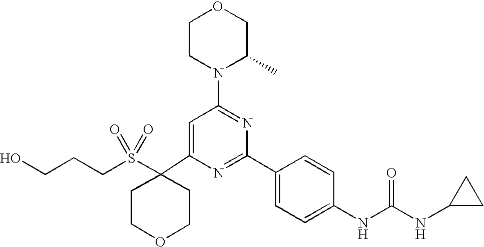 | 3-cyclopropyl-1-[4-[4-[4-(3-hydroxypropylsulfonyl)oxan-4-yl]-6-[(3S)-3-methylmorpholin-4-yl]pyrimidin-2-yl]phenyl]urea | 560 | 1.71 |
| 36cd*** | 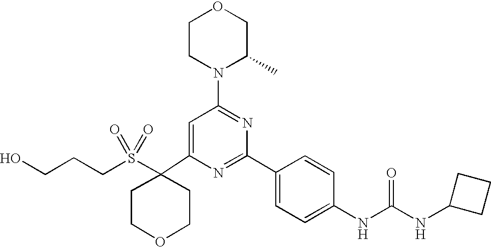 | 3-cyclobutyl-1-[4-[4-[4-(3-hydroxypropylsulfonyl)oxan-4-yl]-6-[(3S)-3-methylmorpholin-4-yl]pyrimidin-2-yl]phenyl]urea | 574 | 1.89 |
| 36ce*** | 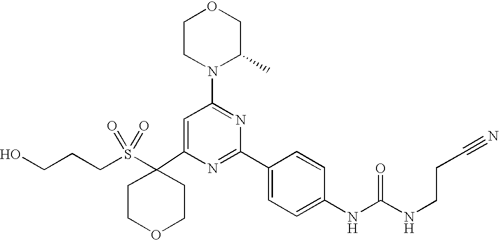 | 3-(2-cyanoethyl)-1-[4-[4-[4-(3-hydroxypropylsulfonyl)oxan-4-yl]-6-[(3S)-3-methylmorpholin-4-yl]pyrimidin-2-yl]phenyl]urea | 573 | 1.62 |
| 36cf*** | 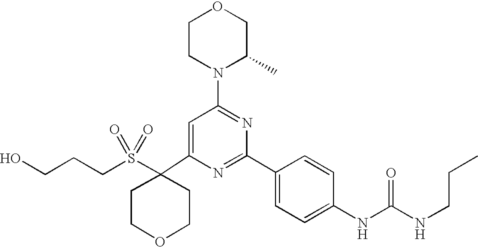 | 3-[4-[4-[4-(3-hydroxypropylsulfonyl)oxan-4-yl]-6-[(3S)-3-methylmorpholin-4-yl]pyrimidin-2-yl]phenyl]-1-propylurea | 562 | 1.83 |
| 36cg*** | 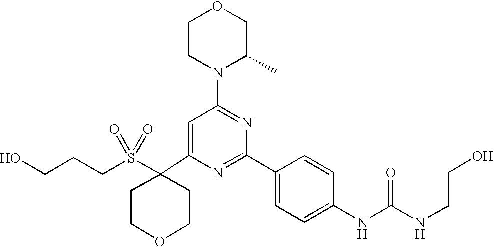 | 3-(2-hydroxyethyl)-1-[4-[4-[4-(3-hydroxypropylsulfonyl)oxan-4-yl]-6-[(3S)-3-methylmorpholin-4-yl]pyrimidin-2-yl]phenyl]urea | 564 | 1.46 |
| 36ch*** | 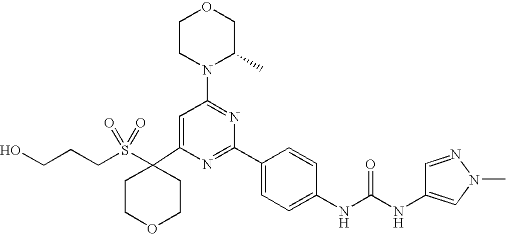 | 1-[4-[4-[4-(3-hydroxypropylsulfonyl)oxan-4-yl]-6-[(3S)-3-methylmorpholin-4-yl]pyrimidin-2-yl]phenyl]-3-(1-methylpyrazol-4-yl)urea | 600 | 1.62 |
| 36ci*** | 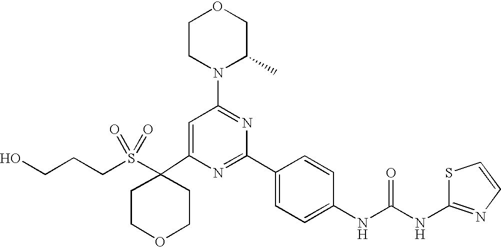 | 1-[4-[4-[4-(3-hydroxypropylsulfonyl)oxan-4-yl]-6-[(3S)-3-methylmorpholin-4-yl]pyrimidin-2-yl]phenyl]-3-(1,3-thiazol-2-yl)urea | 603 | 1.87 |
| 36cj*** | 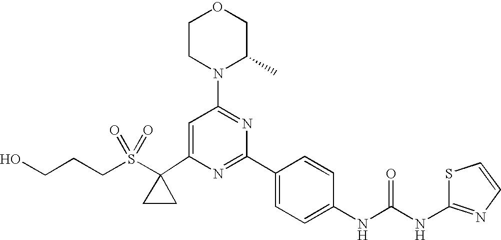 | 1-[4-[4-[1-(3-hydroxypropylsulfonyl)cyclopropyl]-6-[(3S)-3-methylmorpholin-4-yl]pyrimidin-2-yl]phenyl]-3-(1,3-thiazol-2-yl)urea | 559 | 1.94 |
| 36ck*** | 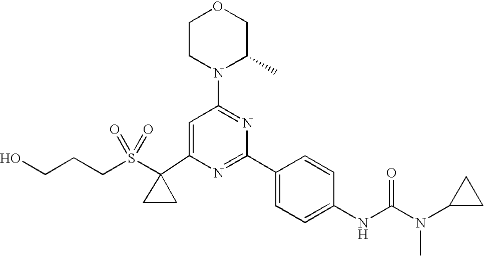 | 1-cyclopropyl-3-[4-[4-[1-(3-hydroxypropylsulfonyl)cyclopropyl]-6-[(3S)-3-methylmorpholin-4-yl]pyrimidin-2-yl]phenyl]-1-methylurea | 530 | 1.96 |
| 36cl*** | 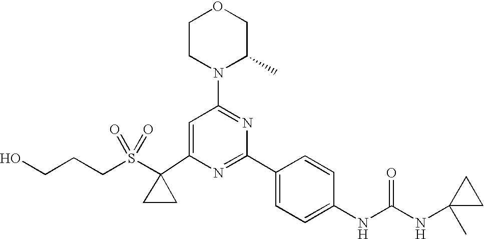 | 1-[4-[4-[1-(3-hydroxypropylsulfonyl)cyclopropyl]-6-[(3S)-3-methylmorpholin-4-yl]pyrimidin-2-yl]phenyl]-3-(1-methylcyclopropyl)urea | 530 | 1.88 |
| 36cm*** | 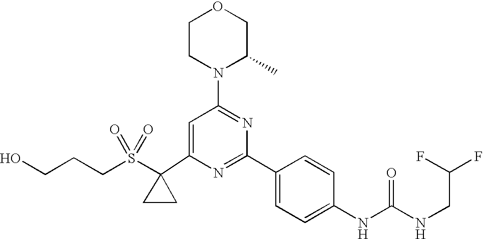 | 3-(2,2-difluoroethyl)-1-[4-[4-[1-(3-hydroxypropylsulfonyl)cyclopropyl]-6-[(3S)-3-methylmorpholin-4-yl]pyrimidin-2-yl]phenyl]urea | 540 | 1.85 |
| 36cn*** | 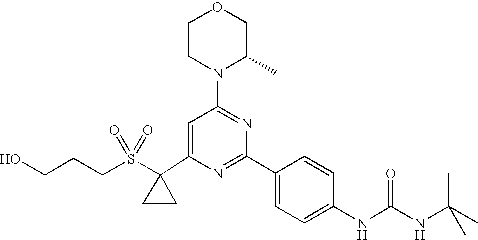 | 3-tert-butyl-1-[4-[4-[1-(3-hydroxypropylsulfonyl)cyclopropyl]-6-[(3S)-3-methylmorpholin-4-yl]pyrimidin-2-yl]phenyl]urea | 532 | 2.10 |
| 36co*** | 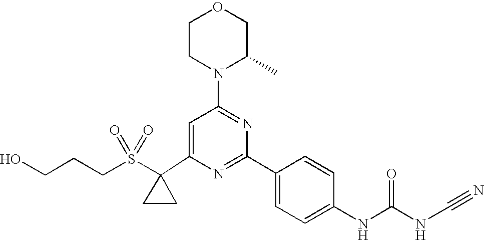 | 3-cyano-1-[4-[4-[1-(3-hydroxypropylsulfonyl)cyclopropyl]-6-[(3S)-3-methylmorpholin-4-yl]pyrimidin-2-yl]phenyl]urea | 501 | 1.12 |
| 36cp*** | 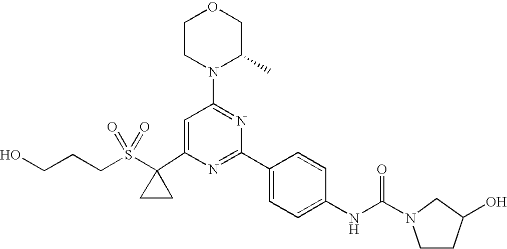 | 3-hydroxy-N-[4-[4-[1-(3-hydroxypropylsulfonyl)cyclopropyl]-6-[(3S)-3-methylmorpholin-4-yl]pyrimidin-2-yl]phenyl]pyrrolidine-1-carboxamide | 546 | 1.52 |
| 36cq*** | 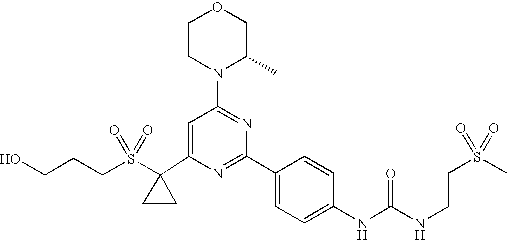 | 1-[4-[4-[1-(3-hydroxypropylsulfonyl)cyclopropyl]-6-[(3S)-3-methylmorpholin-4-yl]pyrimidin-2-yl]phenyl]-3-(2-methylsulfonylethyl)urea | 582 | 1.61 |
| 36cr*** | 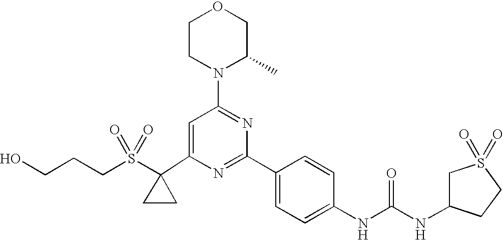 | 3-(1,1-dioxothiolan-3-yl)-1-[4-[4-[1-(3-hydroxypropylsulfonyl)cyclopropyl]-6-[(3S)-3-methylmorpholin-4-yl]pyrimidin-2-yl]phenyl]urea | 594 | 1.66 |
| 36cs*** | 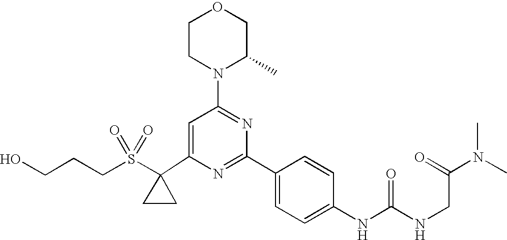 | 2-[[4-[4-[1-(3-hydroxypropylsulfonyl)cyclopropyl]-6-[(3S)-3-methylmorpholin-4-yl]pyrimidin-2-yl]phenyl]carbamoylamino]-N,N-dimethylactamide | 561 | 1.59 |
| 36ct | 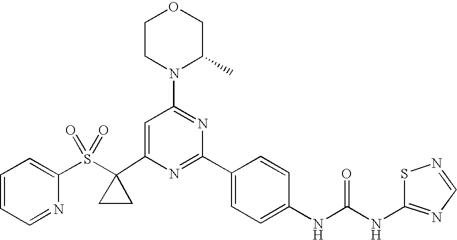 | 1-[4-[4-[(3S)-3-methylmorpholin-4-yl]-6-(1-pyridin-2-ylsulfonylcyclopropyl)pyrimidin-2-yl]phenyl]-3-(1,2,4-thiadiazol-5-yl)urea | 579 | 1.37 |
| 36cu | 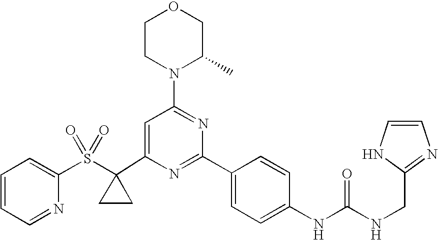 | 3-(1H-imidazol-2-ylmethyl)-1-[4-[4-[(3S)-3-methymorpholin-4-yl]-6-(1-pyridin-2-ylsulfonylcyclopropyl)pyrimidin-2-yl]phenyl]urea | 575 | 1.76 |
| 36cv | 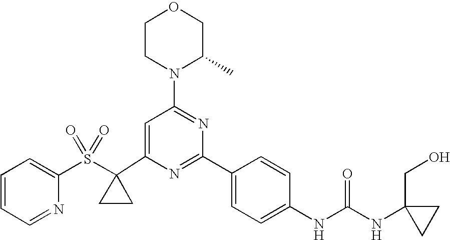 | 3-[1-(hydroxymethyl)cyclopropyl]-1-[4-[4-[(3S)-3-methylmorpholin-4-yl]-6-(1-pyridin-2-ylsulfonylcyclopropyl)pyrimidin-2-yl]phenyl]urea | 565 | 1.77 |
| 36cw | 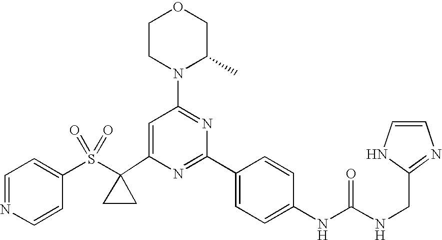 | 3-(1H-imidazol-2-ylmethyl)-1-[4-[4-[(3S)-3-methylmorpholin-4-yl]-6-(1-pyridin-4-ylsulfonylcyclopropyl)pyrimidin-2-yl]phenyl]urea | 576 | 1.77 |
| 36cx | 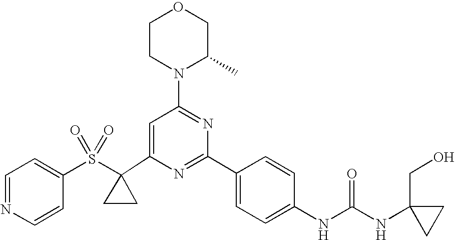 | 3-[1-(hydroxymethyl)cyclopropyl]-1-[4-[4-[(3S)-3-methylmorpholin-4-yl]-6-(1-pyridin-4-ylsulfonylcyclopropyl)pyrimidin-2-yl]phenyl]urea | 566 | 1.78 |
| 36cy | 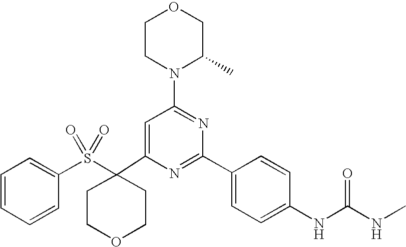 | 1-[4-[4-[4-(benzenesulfonyl)oxan-4-yl]-6-[(3S)-3-methylmorpholin-4-yl]pyrimidin-2-yl]phenyl]-3-methylurea | 553 | 2.14 |
| 36cz | 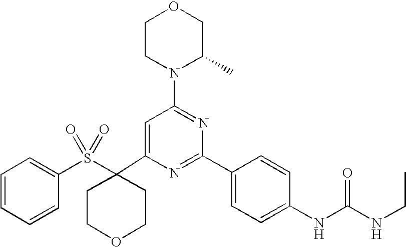 | 3-[4-[4-[4-(benzenesulfonyl)oxan-4-yl]-6-[(3S)-3-methylmorpholin-4-yl]pyrimidin-2-yl]phenyl]-1-ethylurea | 567 | 2.29 |
| 36da | 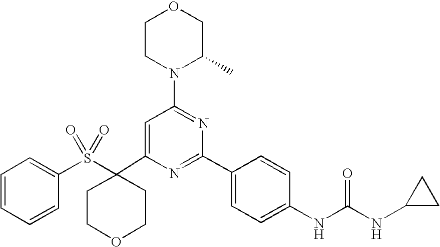 | 1-[4-[4-[4-(benzenesulfonyl)oxan-4-yl]-6-[(3S)-3-methylmorpholin-4-yl]pyrimidin-2-yl]phenyl]-3-cyclopropylurea | 579 | 2.29 |
| 36db | 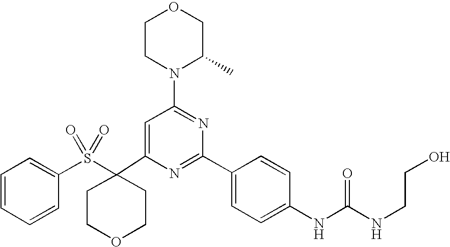 | 1-[4-[4-[4-(benzenesulfonyl)oxan-4-yl]-6-[(3S)-3-methylmorpholin-4-yl]pyrimidin-2-yl]phenyl]-3-(2-hydroxyethyl)urea | 583 | 1.93 |
| 36dc | 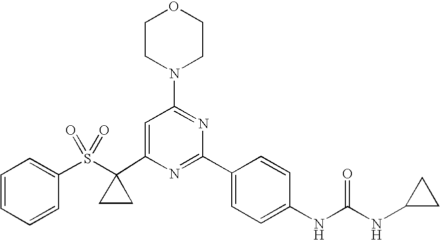 | 1-[4-[4-[1-(benzenesulfonyl)cyclopropyl]-6-morpholin-4-ylpyrimidin-2-yl]phenyl]-3-cyclopropylurea | 520 | 2.24 |
| 36dd | 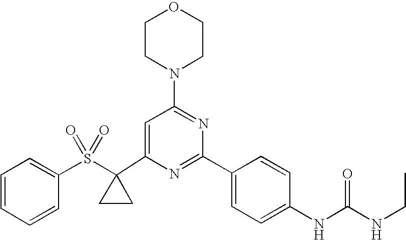 | 3-[4-[4-[1-(benzenesulfonyl)cyclopropyl]-6-morpholin-4-ylpyrimidin-2-yl]phenyl]-1-ethylurea | 508 | 2.24 |
| 36de | 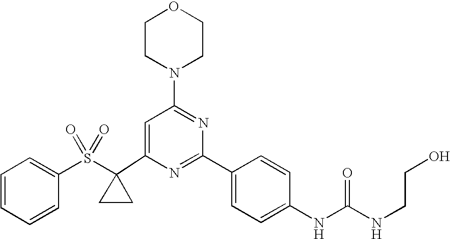 | 1-[4-[4-[1-(benzenesulfonyl)cyclopropyl]-6-morpholin-4-ylpyrimidin-2-yl]phenyl]-3-(2-hydroxyethyl)urea | 524 | 1.89 |
| 36df | 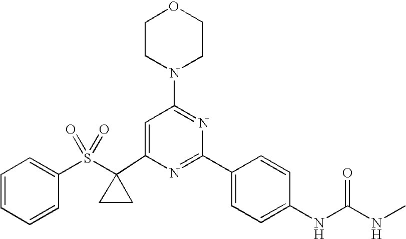 | 3-[4-[4-[1-(benzenesulfonyl)cyclopropyl]-6-morpholin-4-ylpyrimidin-2-yl]phenyl]-1-methylurea | 494 | 2.08 |
| 36dg | 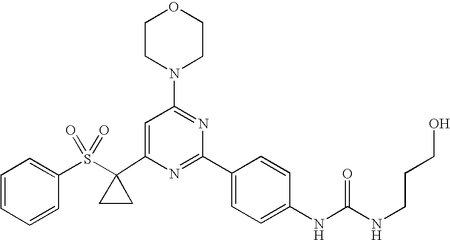 | 1-[4-[4-[1-(benzenesulfonyl)cyclopropyl]-6-morpholin-4-ylpyrimidin-2-yl]phenyl]-3-(3-hydroxypropyl)urea | 538 | 1.95 |
| 36dh | 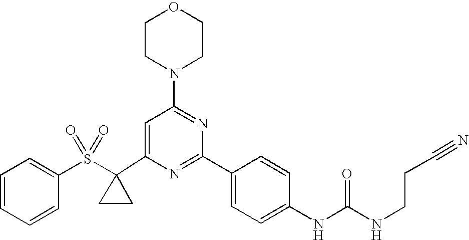 | 1-[4-[4-[1-(benzenesulfonyl)cyclopropyl]-6-morpholin-4-ylpyrimidin-2-yl]phenyl]-3-(2-cyanoethyl)urea | 533 | 2.16 |
| 36di | 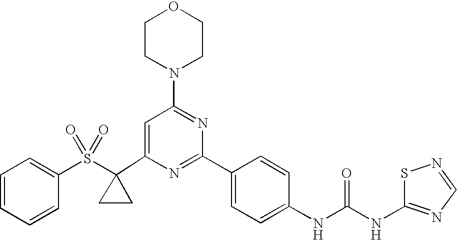 | 1-[4-[4-[1-(benzenesulfonyl)cyclopropyl]-6-morpholin-4-ylpyrimidin-2-yl]phenyl]-3-(1,2,4-thiadiazol-5-yl)urea | 564 | 1.41 |
| 36dj | 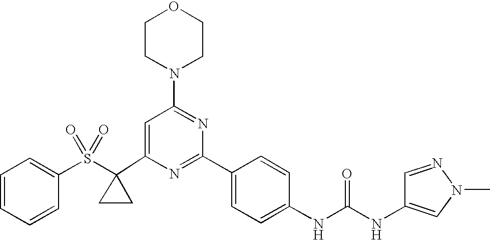 | 3-[4-[4-[1-(benzenesulfonyl)cyclopropyl]-6-morpholin-4-ylpyrimidin-2-yl]phenyl]-1-(1-methylpyrazol-4-yl)urea | 560 | 2.11 |
| 36dk**** | 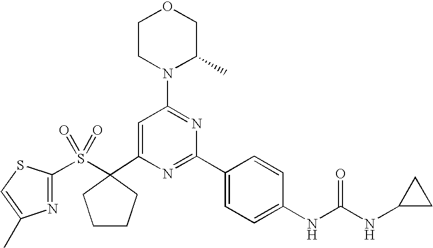 | 3-cyclopropyl-1-[4-[4-[(3S)-3-methylmorpholin-4-yl]-6-[1-[(4-methyl-1,3-thiazol-2-yl)sulfonyl)cyclopentyl]pyrimidin-2-yl]phenyl]urea | 583 | 2.62 |
| 36dl**** | 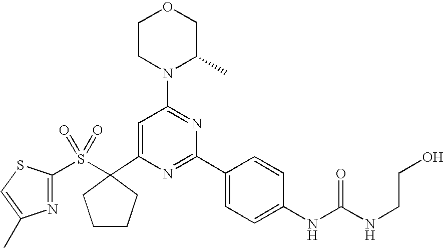 | 3-(2-hydroxyethyl)-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea | 588 | 2.24 |
| 36dm**** | 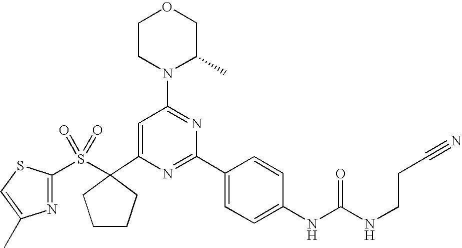 | 3-(2-cyanoethyl)-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea | 596 | 2.50 |
| 36dn**** | 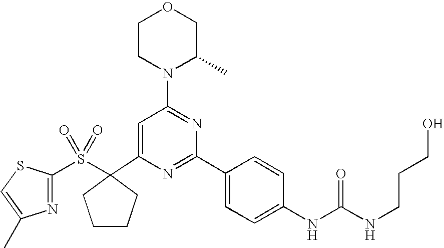 | 3-(3-hydroxypropyl)-1-[4-[4-[(3S)-3-methylmorpholin-4-yl]-6-[1-[(4-methyl-1,3-thiazol-2-yl)sulfonyl]cyclopentyl]pyrimidin-2-yl]phenyl]urea | 601 | 2.28 |
| 36do | 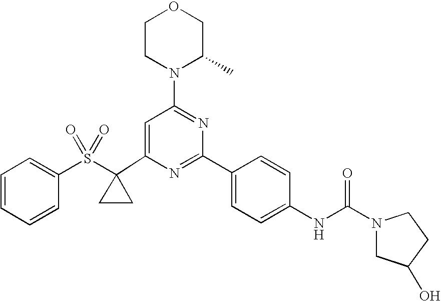 | N-[4-[4-[1-(benzenesulfonyl)cyclopropyl]-6-[(3S)-3-methylmorpholin-4-yl]pyrimidin-2-yl]phenyl]-3-hydroxypyrrolidine-1-carboxamide | 564 | 2.02 |
| 36dp | 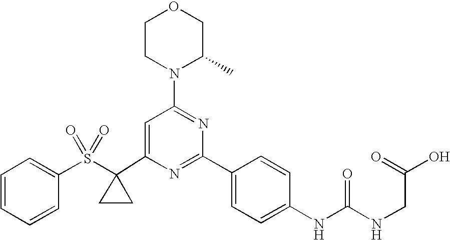 | 2-[[4-[4-[1-(benzenesulfonyl)cyclopropyl]-6-[(3S)-3-methylmorpholin-4-yl]pyrimidin-2-yl]phenyl]carbamoylamino]aceticacid | 552 | 1.21 |
| 36dq | 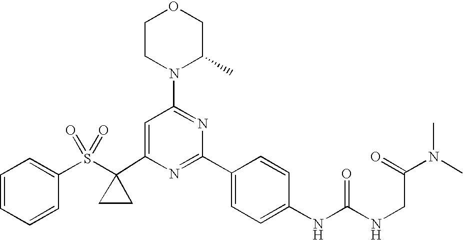 | 2-[[4-[4-[1-(benzenesulfonyl)cyclopropyl]-6-[(3S)-3-methylmorpholin-4-yl]pyrimidin-2-yl]phenyl]carbamoylamino]-N,N-dimethylacetamide | 579 | 2.11 |
| 36dr | 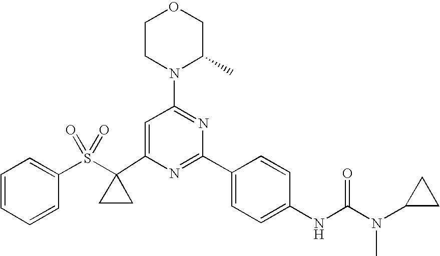 | 3-[4-[4-[1-(benzenesulfonyl)cyclopropyl]-6-[(3S)-3-methylmorpholin-4-yl]pyrimidin-2-yl]phenyl]-1-cyclopropyl-1-methylurea | 548 | 2.61 |
| 36ds | 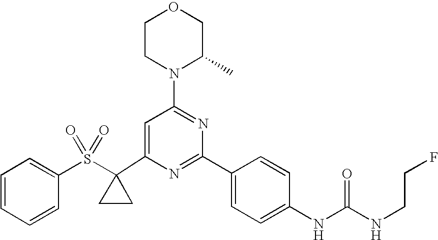 | 1-[4-[4-[1-(benzenesulfonyl)cyclopropyl]-6-[(3S)-3-methylmorpholin-4-yl]pyrimidin-2-yl]phenyl]-3-(2-fluoroethyl)urea | 540 | 2.33 |
| 36dt | 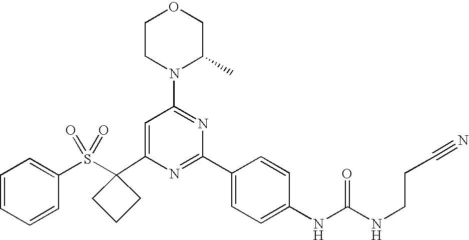 | 1-[4-[4-[1-(benzenesulfonyl)cyclobutyl]-6-[(3S)-3-methylmorpholin-4-yl]pyrimidin-2-yl]phenyl]-3-(2-cyanoethyl)urea | 561 | 2.41 |
| 36du | 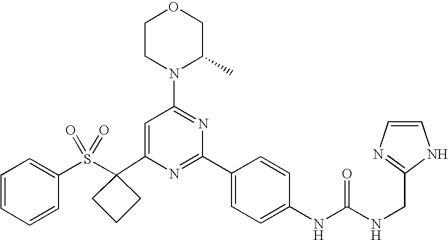 | 1-[4-[4-[1-(benzenesulfonyl)cyclobutyl]-6-[(3S)-3-methylmorpholin-4-yl]pyrimidin-2-yl]phenyl]-3-(1H-imidazol-2-ylmethyl)urea | 588 | 2.31 |
| 36dv | 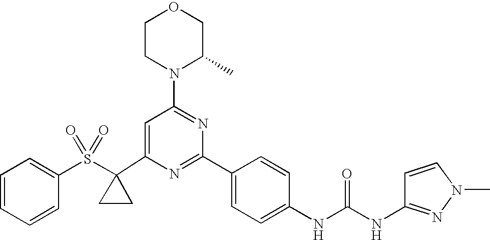 | 1-[4-[4-[1-(benzenesulfonyl)cyclopropyl]-6-[(3S)-3-methylmorpholin-4-yl]pyrimidin-2-yl]phenyl]-3-(1-methypyrazol-3-yl)urea | 574 | 2.76 |
| 36dw | 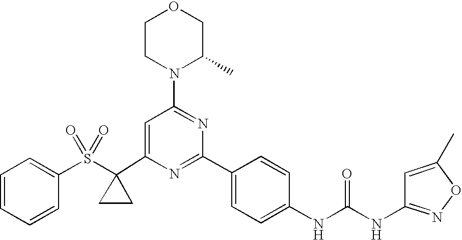 | 3-[4-[4-[1-(benzenesulfonyl)cyclopropyl]-6-[(3S)-3-methylmorpholin-4-yl]pyrimidin-2-yl]phenyl]-1-(5-methyl-1,2-oxazol-3-yl)urea | 575 | 2.97 |
| 36dx | 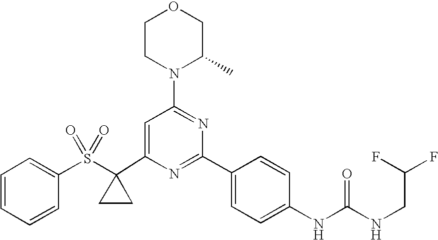 | 1-[4-[4-[1-(benzenesulfonyl)cyclopropyl]-6-[(3S)-3-methylmorpholin-4-yl]pyrimidin-2-yl]phenyl]-3-(2,2-difluoroethyl)urea | 558 | 2.81 |
| 36dy | 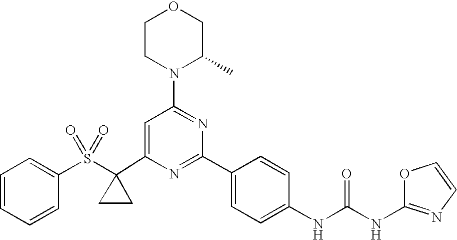 | 1-[4-[4-[1-(benzenesulfonyl)cyclopropyl]-6-[(3S)-3-methylmorpholin-4-yl]pyrimidin-2-yl]phenyl]-3-(1,3-oxazol-2-yl)urea | 561 | 2.51 |
| 36dz | 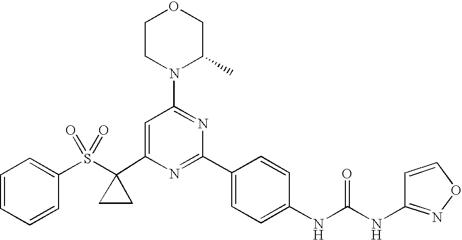 | 1-[4-[4-[1-(benzenesulfonyl)cyclopropyl]-6-[(3S)-3-methylmorpholin-4-yl]pyrimidin-2-yl]phenyl]-3-(1,2-oxazol-3-yl)urea | 561 | 2.85 |
| 36ea | 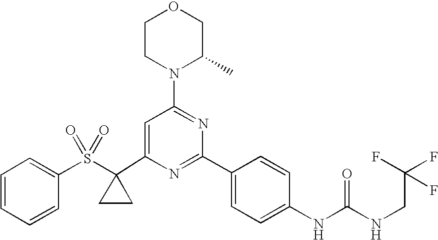 | 1-[4-[4-[1-(benzenesulfonyl)cyclopropyl]-6-[(3S)-3-methylmorpholin-4-yl]pyrimidin-2-yl]phenyl]-3-(2,2,2-trifluroethyl)urea | 576 | 2.94 |
| 36eb | 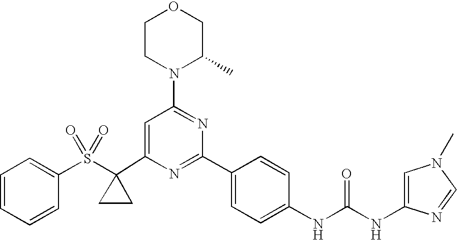 | 1-[4-[4-[1-(benzenesulfonyl)cyclopropyl]-6-[(3S)-3-methylmorpholin-4-yl]pyrimidin-2-yl]phenyl]-3-(1-methylimidazol-4-yl)urea | 574 | 2.61 |
| 36ec | 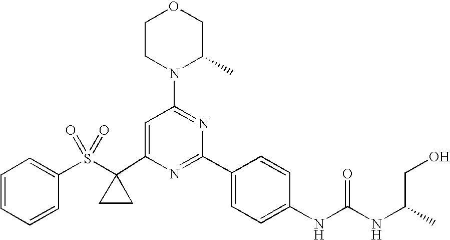 | 1-[4-[4-[1-(benzenesulfonyl)cyclopropyl]-6-[(3S)-3-methylmorpholin-4-yl]pyrimidin-2-yl]phenyl]-3-[(2S)-1-hydroxypropan-2-yl]urea | 552 | 2.47 |
| 36ed | 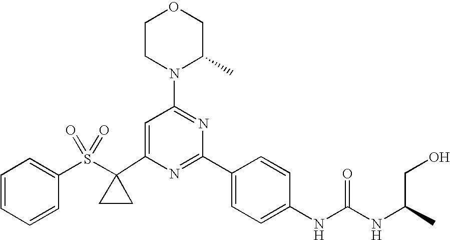 | 1-[4-[4-[1-(benzenesulfonyl)cyclopropyl]-6-[(3S)-3-methylmorpholin-4-yl]pyrimidin-2-yl]phenyl]-3-[(2R)-1-hydroxypropan-2-yl]urea | 552 | 2.47 |
| 36ee | 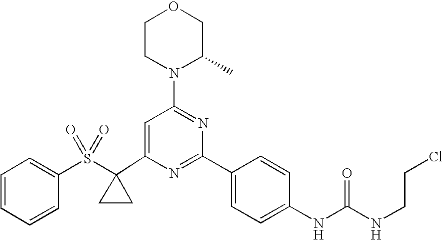 | 1-[4-[4-[1-(benzenesulfonyl)cyclopropyl]-6-[(3S)-3-methylmorpholin-4-yl]pyrimidin-2-yl]phenyl]-3-(2-chloroethyl)urea | 590 | 2.84 |
| 36ef**** | 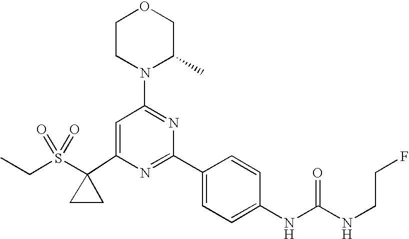 | 1-[4-[4-(1-ethylsulfonylcyclopropyl)-6-[(3S)-3-methylmorpholin-4-yl]pyrimidin-2-yl]phenyl]-3-(2-fluorethyl)urea | 493 | 2.46 |
| 36eg**** | 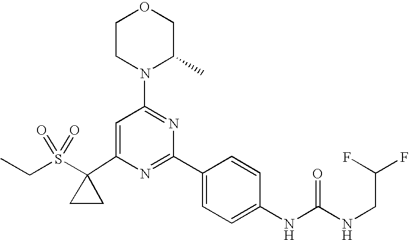 | 3-(2,2-difluoroethyl)-1-[4-[4-(1-ethylsulfonylcyclopropyl)-6-[(3S)-3-methylmorpholin-4-yl]pyrimidin-2-yl]phenyl]urea | 511 | 2.60 |

*Following the initial reaction conditions the mixture was placed in a sealed tube and heated at 100° C. in a microwave reactor for 10 minutes.
**The material was purified by trituration with diethyl ether
***Following the initial reaction conditions the mixture was allowed to cool, tetrabutylammonium fluoride (1 equivalent) added and the reaction stirred at RT for 1 hour. The reaction was purified by prep HPLC.
****The mixture was stirred at RT for 16 hours

### EXAMPLE 36a

¹H NMR (400.132 MHz, DMSO-d₆) δ 0.63-0.68 (2H, m), 0.68-0.73 (2H, m), 1.23 (3H, d), 1.53-1.58 (2H, m), 1.60-1.66 (2H, m), 1.88-1.98 (2H, m), 3.16-3.25 (1H, m), 3.28-3.35 (4H, m), 3.47-3.54 (3H, m), 3.63 (1H, d), 3.76 (1H, d), 3.97 (1H, d), 4.23 (1H, d), 4.56 (1H, s), 4.69 (1H, t), 6.57 (1H, s), 6.77 (1H, s), 7.47 (2H, d), 8.21 (2H, d), 8.68 (1H, s).

mTOR Kinase Assay (Echo): 0.142 μM

### EXAMPLE 36b

¹H NMR (400.132 MHz, DMSO-d₆) δ 1.24 (3H, d), 1.54-1.60 (2H, m), 1.62-1.68 (2H, m), 1.92-1.99 (2H, m), 3.23-3.31 (3H, m), 3.47-3.58 (3H, m), 3.64 (1H, d), 3.77 (1H, d), 3.98 (1H, d), 4.21 (1H, d), 4.57 (1H, s), 4.71 (1H, t), 6.80 (1H, s), 7.64 (2H, d), 8.30 (2H, d), 9.46 (1H, s).

mTOR Kinase Assay (Echo): 0.00179 μM

### EXAMPLE 36c

¹H NMR (400.132 MHz, DMSO-d₆) δ 1.23 (3H, d), 1.52-1.58 (2H, m), 1.62-1.65 (2H, m), 1.89-1.97 (2H, m), 3.22-3.34 (3H, m), 3.44-3.56 (3H, m), 3.63 (1H, d), 3.76 (1H, d), 3.97 (1H, d), 4.16-4.24 (1H, m), 4.35 (2H, d), 4.55 (1H, s), 4.69 (1H, s), 6.67 (1H, t), 6.77 (1H, s), 6.99 (2H, s), 7.52 (2H, d), 8.21 (2H, d), 8.99 (1H, s).

mTOR Kinase Assay (Echo): 0.114 μM

### EXAMPLE 36d

¹H NMR (400.132 MHz, DMSO-d₆) δ 0.70-0.78 (2H, m), 0.81-0.88 (2H, m), 1.23 (3H, d), 1.88-1.96 (1H, m), 2.01-2.14 (1H, m), 2.67 (3H, d), 2.81-2.99 (4H, m), 3.15-3.26 (2H, m), 3.50 (1H, dd), 3.65 (1H, d), 3.77 (1H, d), 3.98 (1H, d), 4.21 (1H, d), 4.55 (1H, s), 6.06 (1H, t), 6.70 (1H, s), 7.50 (2H, d), 8.23 (2H, d), 8.73 (1H, s).

mTOR Kinase Assay (Echo): 0.00317 μM

### EXAMPLE 36e

¹H NMR (400.132 MHz, DMSO-d₆) δ 0.39-0.44 (2H, m), 0.61-0.68 (2H, m), 0.72-0.78 (2H, m), 0.82-0.88 (2H, m), 1.23 (3H, d), 1.84-1.96 (1H, m), 2.02-2.12 (1H, m), 2.50-2.59 (2H, m), 2.81-3.01 (4H, m), 3.17-3.24 (1H, m), 3.50 (1H, dd), 3.65 (1H, d), 3.77 (1H, d), 3.98 (1H, d), 4.22 (1H, d), 4.55 (1H, s), 6.42 (1H, t), 6.71 (1H, s), 7.48 (2H, d), 8.24 (2H, d), 8.53 (1H, s).

mTOR Kinase Assay (Echo): 0.00667 μM

### EXAMPLE 36f

¹H NMR (400.132 MHz, DMSO-d₆) δ 0.68-0.80 (2H, m), 0.81-0.89 (2H, m), 1.23 (3H, d), 1.84-1.95 (1H, m), 2.02-2.13 (1H, m), 2.76-3.00 (4H, m), 3.11-3.27 (3H, m), 3.43-3.56 (3H, m), 3.65 (1H, d), 3.77 (1H, d), 3.98 (1H, d), 4.21 (1H, d), 4.54 (1H, s), 4.72 (1H, t), 6.24 (1H, t), 6.70 (1H, s), 7.48 (2H, d), 8.24 (2H, d), 8.79 (1H, s).

mTOR Kinase Assay (Echo): 0.00278 μM

### EXAMPLE 36g

¹H NMR (400.132 MHz, DMSO-d₆) δ 0.69-0.79 (2H, m), 0.81-0.89 (2H, m), 1.23 (3H, d), 1.84-1.96 (1H, m), 2.02-2.12 (1H, m), 2.63-2.73 (2H, m), 2.83-3.03 (4H, m), 3.14-3.23 (1H, m), 3.33-3.43 (2H, m), 3.51 (1H, dd), 3.65 (1H, d), 3.77 (1H, d), 3.98 (1H, d), 4.22 (1H, d), 4.55 (1H, s), 6.51 (1H, t), 6.71 (1H, s), 7.51 (2H, d), 8.25 (2H, d), 8.90 (1H, s).

mTOR Kinase Assay (Echo): 0.00841 μM

### EXAMPLE 36h

¹H NMR (400.132 MHz, DMSO-d₆) δ 0.70-0.80 (2H, m), 0.82-0.91 (2H, m), 1.23 (3H, d), 1.86-1.97 (1H, m), 2.02-2.12 (1H, m), 2.77-3.03 (4H, m), 3.15-3.29 (5H, m), 3.51 (1H, d), 3.66 (1H, d), 3.77 (1H, d), 3.98 (1H, d), 4.22 (1H, d), 4.55 (1H, s), 6.72 (1H, s), 7.38 (1H, s), 7.54 (2H, d), 7.76 (1H, s), 8.27 (2H, d), 8.37 (1H, s), 8.83 (1H, s).

mTOR Kinase Assay (Echo): 0.00314 μM

### EXAMPLE 36i

¹H NMR (400.132 MHz, DMSO-d₆) δ 0.74-0.84 (2H, m), 0.87-0.94 (2H, m), 1.28 (3H, d), 1.91-2.01 (1H, m), 2.08-2.19 (1H, m), 2.84-3.09 (4H, m), 3.25-3.32 (2H, m), 3.56 (1H, d), 3.71 (1H, d), 3.82 (1H, d), 4.03 (1H, d), 4.27 (1H, d), 4.38 (2H, d), 4.61 (1H, s), 6.68 (1H, t), 6.76 (1H, s), 7.00 (1H, s), 7.56 (2H, d), 8.31 (2H, d), 8.98 (1H, s).

mTOR Kinase Assay (Echo): 0.0518 μM

### EXAMPLE 36j

¹H NMR (400.132 MHz, DMSO-d₆) δ 0.39-0.43 (2H, m), 0.62-0.67 (2H, m), 0.68-0.75 (2H, m), 0.80-0.87 (2H, m), 1.22 (3H, d), 1.52-1.61 (2H, m), 1.76-1.86 (2H, m), 2.50-2.61 (4H, m), 2.74-2.90 (2H, m), 3.14-3.24 (1H, m), 3.50 (2H, dd), 3.65 (1H, d), 3.77 (1H, d), 3.98 (1H, d), 4.22 (1H, d), 4.54 (1H, s), 6.42 (1H, s), 6.81 (1H, s), 7.50 (2H, d), 8.24 (2H, d), 8.53 (1H, s).

mTOR Kinase Assay (Echo): 0.018 μM

### EXAMPLE 36k

¹H NMR (400.132 MHz, DMSO-d₆) δ 0.66-0.77 (2H, m), 0.80-0.89 (2H, m), 1.22 (3H, d), 1.52-1.61 (2H, m), 1.76-1.86 (2H, m), 2.43-2.61 (3H, m), 2.75-2.89 (2H, m), 3.16-3.22 (3H, m), 3.43-3.55 (3H, m), 3.65 (1H, d), 3.77 (1H, d), 3.98 (1H, d), 4.22 (1H, d), 4.54 (1H, s), 4.73 (1H, t), 6.25 (1H, t), 6.81 (1H, s), 7.49 (2H, d), 8.24 (2H, d), 8.79 (1H, s).

mTOR Kinase Assay (Echo): 0.0131 μM

### EXAMPLE 36l

¹H NMR (400.132 MHz, DMSO-d₆) δ 0.66-0.76 (2H, m), 0.82-0.87 (2H, m), 1.22 (3H, d), 1.53-1.60 (2H, m), 1.74-1.85 (2H, m), 2.49-2.61 (3H, m), 2.65-2.72 (2H, m), 2.74-2.88 (2H, m), 3.16-3.25 (1H, m), 3.33-3.40 (2H, m), 3.50 (1H, d), 3.65 (1H, d), 3.77 (1H, d), 3.98 (1H, d), 4.23 (1H, d), 4.55 (1H, s), 6.52 (1H, t), 6.81 (1H, s), 7.51 (2H, d), 8.26 (2H, d), 8.91 (1H, s).

mTOR Kinase Assay (Echo): 0.0428 μM

### EXAMPLE 36m

¹H NMR (400.132 MHz, DMSO-d₆) δ 0.67-0.77 (2H, m), 0.81-0.89 (2H, m), 1.22 (3H, d), 1.52-1.64 (4H, m), 1.78-1.85 (2H, m), 2.41-2.56 (3H, m), 2.74-2.93 (2H, m), 3.13-3.25 (3H, m), 3.42-3.56 (3H, m), 3.65 (1H, d), 3.77 (1H, d), 3.98 (1H, d), 4.16-4.26 (1H, m), 4.47 (1H, t), 4.54 (1H, s), 6.19 (1H, t), 6.81 (1H, s), 7.49 (2H, d), 8.24 (2H, d), 8.70 (1H, s).

mTOR Kinase Assay (Echo): 0.0347 μM

### EXAMPLE 36n

¹H NMR (400.132 MHz, DMSO-d₆) δ 1.21 (3H, d), 1.76-1.80 (2H, m), 1.93-1.97 (2H, m), 2.48 (3H, s), 3.14-3.21 (1H, m), 3.47 (1H, t), 3.62 (1H, d), 3.76 (1H, d), 3.97 (1H, d), 4.14-4.17 (1H, m), 4.32 (2H, d), 4.42-4.49 (1H, m), 6.61 (1H, t), 6.78 (1H, s), 6.93 (2H, bs), 7.43 (2H, d), 7.84 (1H, s), 7.90 (2H, d), 8.90 (1H, s), 11.85 (1H, s).

mTOR Kinase Assay (Echo): 0.0248 μM

### EXAMPLE 36o

¹H NMR (400.132 MHz, DMSO-d₆) δ 1.21 (3H, d), 1.77-1.79 (2H, m), 1.94-1.97 (2H, m), 2.48 (3H, s), 2.70 (2H, t), 3.14-3.22 (1H, m), 3.35-3.39 (2H, m), 3.44-3.50 (1H, m), 3.60-3.64 (1H, m), 3.76 (1H, d), 3.95-3.99 (1H, m), 4.15-4.18 (1H, m), 4.42-4.48 (1H, m), 6.51 (1H, t), 6.78 (1H, s), 7.43 (2H, d), 7.84 (1H, s), 7.90 (2H, d), 8.90 (1H, s).

mTOR Kinase Assay (Echo): 0.00358 μM

### EXAMPLE 36p

¹H NMR (400.132 MHz, DMSO-d₆) δ 1.22 (3H, d), 1.77-1.81 (2H, m), 1.95-1.99 (2H, m), 2.51 (3H, s), 3.16-3.24 (1H, m), 3.45-3.51 (1H, m), 3.61-3.65 (1H, m), 3.77 (1H, d), 3.96-4.00 (1H, m), 4.16-4.20 (1H, m), 4.43-4.49 (1H, m), 6.82 (1H, s), 7.55 (2H, d), 7.87 (1H, s), 8.00 (2H, d), 8.37 (1H, s), 9.45 (1H, s), 11.32 (1H, s).

mTOR Kinase Assay (Echo): 0.00161 μM

### EXAMPLE 36q

¹H NMR (400.132 MHz, DMSO-d₆) δ 0.63-0.66 (2H, m), 0.69-0.73 (2H, m), 1.21 (3H, d), 1.77-1.80 (2H, m), 1.94-1.97 (2H, m), 2.48 (3H, s), 3.14-3.21 (1H, m), 3.44 (2H, d), 3.47-3.50 (1H, m), 3.60-3.64 (1H, m), 3.76 (1H, d), 3.95-3.98 (1H, m), 4.14-4.17 (1H, m), 4.42-4.48 (1H, m), 4.83 (1H, s), 6.54 (1H, s), 6.77 (1H, s), 7.40 (2H, d), 7.84 (1H, s), 7.89 (2H, d), 8.65 (1H, s).

mTOR Kinase Assay (Echo): 0.0889 μM

### EXAMPLE 36r

¹H NMR (400.132 MHz, DMSO-d₆) δ 1.54-1.57 (2H, m), 1.65-1.68 (2H, m), 2.70 (2H, t), 3.27 (3H, s), 3.35-3.40 (2H, m), 3.72 (8H, s), 6.53 (1H, t), 6.81 (1H, s), 7.50-7.54 (2H, m), 8.20-8.24 (2H, m), 8.93 (1H, s).

mTOR Kinase Assay (Echo): 0.00733 μM

### EXAMPLE 36s

¹H NMR (400.132 MHz, DMSO-d₆) δ 1.55-1.59 (2H, m), 1.67-1.70 (2H, m), 3.27 (3H, s), 3.73 (8H, s), 6.86 (1H, s), 7.61-7.65 (2H, m), 8.30-8.33 (2H, m), 8.37 (1H, s), 9.44 (1H, s), 11.40 (1H, s).

mTOR Kinase Assay (Echo): 0.00475 μM

### EXAMPLE 36t

¹H NMR (400.132 MHz, DMSO-d₆) δ 1.54-1.57 (2H, m), 1.65-1.68 (2H, m), 3.27 (3H, s), 3.72 (8H, s), 4.32 (2H, d), 6.62 (1H, t), 6.81-6.83 (2H, m), 7.03 (1H, s), 7.50-7.54 (2H, m), 8.20-8.23 (2H, m), 8.94 (1H, s), 11.83 (1H, s).

mTOR Kinase Assay (Echo): 0.194 μM

### EXAMPLE 36u

¹H NMR (400.132 MHz, DMSO-d₆) δ 1.23 (3H, d), 1.32 (3H, t), 1.53-1.56 (2H, m), 1.62-1.65 (2H, m), 2.70 (2H, t), 3.17-3.24 (1H, m), 3.35-3.52 (5H, m), 3.63 (1H, dd), 3.76 (1H, d), 3.97 (1H, dd), 4.17-4.24 (1H, m), 4.52-4.58 (1H, m), 6.53 (1H, t), 6.78 (1H, s), 7.50-7.54 (2H, m), 8.18-8.22 (2H, m), 8.92 (1H, s).

mTOR Kinase Assay (Echo): 0.00922 μM

### EXAMPLE 36v

¹H NMR (400.132 MHz, DMSO-d₆) δ 0.63-0.67 (2H, m), 0.69-0.73 (2H, m), 1.23 (3H, d), 1.32 (3H, t), 1.53-1.58 (2H, m), 1.60-1.65 (2H, m), 3.17-3.24 (1H, m), 3.40-3.52 (5H, m), 3.63 (1H, dd), 3.76 (1H, d), 3.97 (1H, dd), 4.17-4.24 (1H, m), 4.52-4.58 (1H, m), 4.81-4.86 (1H, m), 6.57 (1H, s), 6.78 (1H, s), 7.46-7.50 (2H, m), 8.17-8.21 (2H, m), 8.68 (1H, s).

mTOR Kinase Assay (Echo): 0.467 μM

### EXAMPLE EXAMPLE 36w

¹H NMR (400.132 MHz, DMSO-d₆) δ 1.23 (3H, d), 1.32 (3H, t), 1.53-1.56 (2H, m), 1.62-1.65 (2H, m), 3.17-3.24 (1H, m), 3.40-3.52 (3H, m), 3.63 (1H, dd), 3.76 (1H, d), 3.97 (1H, dd), 4.18-4.24 (1H, m), 4.32 (2H, d), 4.53-4.58 (1H, m), 6.63 (1H, t), 6.78 (1H, s), 6.84 (1H, s), 7.03 (1H, s), 7.50-7.54 (2H, m), 8.18-8.22 (2H, m), 8.93 (1H, s), 11.84 (1H, s).

mTOR Kinase Assay (Echo): 0.11 μM

### EXAMPLE 36x

¹H NMR (400.132 MHz, DMSO-d₆) δ 0.63-0.66 (2H, m), 1.18 (2H, d), 1.59-1.62 (3H, m), 1.88-1.90 (2H, m), 2.09 (2H, s), 3.15 (1H, dt), 3.30 (2H, s), 3.44-3.49 (1H, m), 3.61 (1H, dd), 3.75 (1H, d), 3.96 (1H, dd), 4.14 (1H, d), 4.41 (1H, s), 4.83 (1H, s), 6.53 (1H, s), 6.65 (1H, s), 7.36-7.44 (4H, m), 7.80-7.86 (4H, m), 8.64 (1H, s).

mTOR Kinase Assay (Echo): 0.182 μM

### EXAMPLE 36y

¹H NMR (400.132 MHz, DMSO-d₆) δ 1.19 (3H, d), 1.59-1.62 (2H, m), 1.88-1.91 (2H, m), 2.70 (2H, t), 3.13-3.18 (1H, m), 3.33-3.39 (2H, m), 3.46 (1H, t), 3.61 (1H, d), 3.75 (1H, d), 3.96 (1H, d), 4.14 (1H, d), 4.43 (1H, s), 6.49 (1H, t), 6.65 (1H, s), 7.40-7.44 (4H, m), 7.81-7.86 (4H, m), 8.89 (1H, s).

mTOR Kinase Assay (Echo): 0.00131 μM

### EXAMPLE 36z

¹H NMR (400.132 MHz, DMSO-d₆) δ 1.20 (3H, d), 1.62-1.64 (2H, m), 1.90-1.94 (2H, m), 3.17-3.18 (1H, m), 3.42-3.51 (1H, m), 3.62 (1H, d), 3.76 (1H, d), 3.94-3.98 (1H, m), 4.16 (1H, d), 4.44 (1H, s), 6.69 (1H, s), 7.43 (2H, t), 7.52 (2H, d), 7.83-7.87 (2H, m), 7.92 (2H, d), 8.37 (1H, s), 9.39 (1H, s), 11.34 (1H, s).

mTOR Kinase Assay (Echo): 0.00208 μM

### EXAMPLE 36aa

¹H NMR (400.132 MHz, DMSO-d₆) δ 1.19 (3H, d), 1.59-1.62 (2H, m), 1.88-1.91 (2H, m), 3.12-3.19 (1H, m), 3.43-3.49 (1H, m), 3.59-3.63 (1H, m), 3.75 (1H, d), 3.96 (1H, dd), 4.14 (1H, d), 4.32 (2H, d), 4.42 (1H, s), 6.60 (1H, t), 6.65 (1H, s), 6.94 (2H, s), 7.42 (4H, t), 7.81-7.86 (4H, m), 8.91 (1H, s), 11.89 (1H, s).

mTOR Kinase Assay (Echo): 0.0749 μM

### EXAMPLE 36ab

¹H NMR (400.132 MHz, DMSO-d₆) δ 0.40-0.43 (2H, m), 0.62-0.67 (2H, m), 1.19 (3H, d), 1.51-1.60 (2H, m), 1.80-1.89 (2H, m), 2.53-2.58 (1H, m), 2.70-2.80 (4H, m), 3.12 (1H, dt), 3.47 (1H, dt), 3.63 (1H, dd), 3.75 (1H, d), 3.96 (1H, dd), 4.10 (1H, d), 4.46 (1H, d), 6.41 (1H, d), 6.59 (1H, s), 7.37 (2H, d), 7.54-7.56 (1H, m), 7.57-7.59 (1H, m), 7.76 (2H, d), 7.85 (1H, dt), 8.48 (1H, s), 8.73-8.75 (1H, m).

mTOR Kinase Assay (Echo): 0.011 μM

### EXAMPLE 36ac

¹H NMR (400.132 MHz, DMSO-d₆) δ 1.19 (3H, d), 1.52-1.58 (2H, m), 1.81-1.89 (2H, m), 2.68-2.80 (4H, m), 3.08-3.15 (2H, m), 3.27 (1H, s), 3.44-3.50 (3H, m), 3.62 (1H, dd), 3.75 (1H, d), 3.96 (1H, dd), 4.10 (1H, d), 4.47 (1H, s), 4.72 (1H, t), 6.23 (1H, t), 6.59 (1H, s), 7.36 (2H, d), 7.54-7.59 (2H, m), 7.76 (2H, d), 7.85 (1H, dt), 8.73-8.75 (2H, m).

mTOR Kinase Assay (Echo): 0.00441 μM

### EXAMPLE 36ad

¹H NMR (400.132 MHz, DMSO-d₆) δ 8.87 (1H, s), 8.74 (1H, d), 7.85 (1H, td), 7.77 (2H, d), 7.54-7.59 (2H, m), 7.38 (2H, d), 6.60 (1H, s), 6.51 (1H, t), 4.48 (1H, s), 4.10 (1H, d), 3.96 (1H, dd), 3.75 (1H, d), 3.63 (1H, dd), 3.47 (1H, td), 3.37 (2H, q), 3.12 (1H, td), 2.72-2.81 (4H, m), 2.70 (2H, t), 1.80-1.89 (2H, m), 1.55-1.58 (2H, m), 1.19 (3H, d).

mTOR Kinase Assay (Echo): 0.0323 μM

### EXAMPLE 36ae

¹H NMR (400.132 MHz, DMSO-d₆) δ 1.19 (3H, d), 1.55-1.63 (4H, m), 1.81-1.89 (2H, m), 2.68-2.80 (4H, m), 3.08-3.19 (3H, m), 3.45-3.49 (3H, m), 3.62 (1H, dd), 3.75 (1H, d), 3.96 (1H, dd), 4.10 (1H, d), 4.48 (2H, t), 6.18 (1H, t), 6.59 (1H, s), 7.36 (2H, d), 7.53-7.59 (2H, m), 7.75 (2H, d), 7.85 (1H, dt), 8.65 (1H, s), 8.74 (1H, d).

mTOR Kinase Assay (Echo): 0.0151 μM

### EXAMPLE 36af

¹H NMR (400.132 MHz, DMSO-d₆) δ 0.40-0.43 (2H, m), 0.62-0.67 (2H, m), 1.18 (3H, d), 1.88-1.95 (1H, m), 2.11-2.17 (1H, m), 2.53-2.57 (2H, m), 2.79-2.87 (2H, m), 3.12 (1H, dt), 3.23-3.28 (3H, m), 3.46 (1H, dt), 3.62 (1H, dd), 3.74 (1H, d), 3.95 (1H, dd), 4.09 (1H, d), 4.44 (1H, s), 6.40 (1H, d), 6.48 (1H, s), 7.37 (2H, d), 7.58-7.61 (2H, m), 7.87 (1H, ddt), 8.48 (1H, s), 8.74-8.75 (1H, m).

mTOR Kinase Assay (Echo): 0.00275 μM

### EXAMPLE 36ag

¹H NMR (400.132 MHz, DMSO-d₆) δ 1.18 (3H, d), 1.86-1.97 (1H, m), 2.09 -2.18 (1H, m), 2.65 (3H, s), 2.79-2.87 (2H, m), 3.12 (1H, dt), 3.22-3.28 (2H, m), 3.46 (1H, dt), 3.61 (1H, dd), 3.74 (1H, d), 3.95 (1H, dd), 4.08 (1H, d), 4.44 (1H, s), 6.04-6.07 (1H, m), 6.48 (1H, s), 7.37 (2H, d), 7.58-7.61 (2H, m), 7.74 (2H, d), 7.87 (1H, dt), 8.68 (1H, s), 8.75 (1H, d).

mTOR Kinase Assay (Echo): 0.0013 μM

### EXAMPLE 36ah

¹H NMR (400.132 MHz, DMSO-d₆) δ 1.18 (3H, d), 1.88-1.95 (1H, m), 2.10-2.17 (1H, m), 2.81-2.87 (2H, m), 3.08-3.19 (2H, m), 3.22-3.27 (3H, m), 3.44-3.50 (3H, m), 3.62 (1H, dd), 3.74 (1H, d), 3.95 (1H, dd), 4.08 (1H, d), 4.45 (1H, s), 4.72 (1H, t), 6.23 (1H, t), 6.48 (1H, s), 7.35 (2H, d), 7.60 (2H, t), 7.74 (2H, d), 7.87 (1H, t), 8.75 (2H, s).

mTOR Kinase Assay (Echo): 0.00247 μM

### EXAMPLE 36ai

¹H NMR (400.132 MHz, DMSO-d₆) δ 1.18 (3H, d), 1.88-1.95 (1H, m), 2.09-2.16 (1H, m), 2.69 (2H, t), 2.78-2.87 (2H, m), 3.10-3.16 (1H, m), 3.23-3.25 (3H, m), 3.35-3.39 (2H, m), 3.47 (1H, t), 3.62 (1H, dd), 3.74 (1H, d), 3.95 (1H, d), 4.09 (1H, d), 4.45 (1H, s), 6.49 (1H, s), 7.38 (2H, d), 7.58-7.61 (2H, m), 7.76 (2H, d), 7.87 (1H, dt), 8.74-8.75 (1H, m), 8.86 (1H, s).

mTOR Kinase Assay (Echo): 0.00561 μM

### EXAMPLE 36aj

¹H NMR (400.132 MHz, DMSO-d₆) δ 1.18 (3H, d), 1.89-1.97 (1H, m), 2.11-2.18 (1H, m), 2.79-2.87 (2H, m), 3.13 (1H, dt), 3.23-3.29 (2H, m), 3.47 (1H, dt), 3.62 (1H, dd), 3.75 (1H, d), 3.79 (3H, s), 3.96 (1H, dd), 4.10 (1H, d), 4.45 (1H, s), 6.50 (1H, s), 7.38 (1H, s), 7.41 (2H, d), 7.59-7.62 (1H, m), 7.76 (1H, s), 7.77 (2H, d), 7.86-7.90 (1H, m), 7.88 (1H, dt), 8.38 (1H, s), 8.75-8.76 (1H, m), 8.80 (1H, s).

mTOR Kinase Assay (Echo): 0.00258 μM

### EXAMPLE 36ak

¹H NMR (400.132 MHz, DMSO-d₆) δ 1.19 (3H, d), 1.89-1.98 (1H, m), 2.10-2.19 (1H, m), 2.79-2.87 (2H, m), 3.13 (1H, dt), 3.23-3.28 (2H, m), 3.47 (1H, dt), 3.62 (1H, dd), 3.74 (2H, s), 3.75 (1H, d), 3.96 (1H, dd), 4.10 (1H, d), 4.45 (1H, s), 6.25 (1H, d), 6.51 (1H, s), 7.43 (2H, d), 7.54 (1H, d), 7.59-7.63 (1H, m), 7.60 (1H, d), 7.79 (2H, d), 7.88 (1H, dt), 8.75-8.76 (1H, m), 8.91 (1H, s), 9.11 (1H, s).

mTOR Kinase Assay (Echo): 0.00245 μM

### EXAMPLE 36al

¹H NMR (400.132 MHz, DMSO-d₆) δ 0.41 (2H, m), 0.63-0.66 (2H, m), 1.59 (2H, m), 1.88 (2H, m), 2.57-2.61 (1H, m), 3.66 (4H, s), 3.68 (4H, s), 6.39 (1H, s), 6.71 (1H, s), 7.38-7.43 (4H, m), 7.78 (2H, d), 7.85 (2H, t), 8.52 (1H, s).

mTOR Kinase Assay (Echo): 0.0187 μM

### EXAMPLE 36am

¹H NMR (400.132 MHz, DMSO-d₆) δ 1.58-1.61 (2H, m), 1.87-1.90 (2H, m), 2.70 (2H, t), 3.37 (2H, q), 3.67 (4H, s), 3.69 (4H, s), 6.49 (1H, t), 6.72 (1H, s), 7.39-7.44 (4H, m), 7.79-7.86 (4H, m), 8.90 (1H, s).

mTOR Kinase Assay (Echo): 0.0277 μM

### EXAMPLE 36an

¹H NMR (400.132 MHz, DMSO-d₆) δ 1.59-1.62 (2H, m), 1.89-1.92 (2H, m), 3.68 (4H, s), 3.70 (4H, s), 6.76 (1H, s), 7.43 (2H, t), 7.51 (2H, d), 7.83-7.96 (4H, m), 8.37 (1H, s), 9.39 (1H, s), 11.33 (1H, s).

mTOR Kinase Assay (Echo): 0.0104 μM

### EXAMPLE 36ao

¹H NMR (400.132 MHz, DMSO-d₆) δ 1.06 (3H, t), 1.57-1.61 (2H, m), 1.87-1.90 (2H, m), 3.09-3.16 (2H, m), 3.65-3.66 (4H, m), 3.69-3.70 (4H, m), 6.12 (1H, t), 6.71 (1H, s), 7.37-7.44 (4H, m), 7.78 (2H, d), 7.82-7.86 (2H, m), 8.64 (1H, s)

### EXAMPLE 36ap

¹H NMR (400.132 MHz, DMSO-d₆) δ 1.57-1.61 (2H, m), 1.87-1.90 (2H, m), 2.66 (3H, d), 3.66 (4H, s), 3.69-3.70 (4H, m), 6.01-6.05 (1H, m), 6.71 (1H, s), 7.38-7.43 (4H, m), 7.78 (2H, d), 7.82-7.86 (2H, m), 8.72 (1H, s)

### EXAMPLE 36aq

¹H NMR (400.132 MHz, DMSO-d₆) δ 1.58-1.61 (2H, m), 1.88-1.91 (2H, m), 3.63-3.67 (4H, m), 3.69-3.73 (4H, m), 3.79 (3H, s), 6.73 (1H, s), 7.38-7.45 (5H, m), 7.77-7.86 (5H, m), 8.34 (1H, s), 8.82 (1H, s)

### EXAMPLE 36ar

¹H NMR (400.132 MHz, DMSO-d₆) δ 1.66-1.69 (2H, m), 1.95-1.98 (2H, m), 3.15-3.19 (2H, m), 3.44-3.48 (2H, m), 3.69 (8H, s), 4.72 (1H, t), 6.22 (1H, t), 6.73 (1H, s), 7.34 (2H, d), 7.63 (2H, d), 7.77 (2H, d), 8.77 (1H, s), 8.86 (2H, d)

### EXAMPLE 36as

¹H NMR (400.132 MHz, DMSO-d₆) δ 1.66-1.70 (2H, m), 1.96-1.99 (2H, m), 3.69 (8H, s), 3.79 (3H, s), 6.74 (1H, s), 7.40 (3H, d), 7.66 (2H, d), 7.77-7.78 (3H, m), 8.35 (1H, s), 8.80 (1H, s), 8.87 (2H, d).

mTOR Kinase Assay (Echo): 0.00911 μM

### EXAMPLE 36at

¹H NMR (400.132 MHz, DMSO-d₆) δ 1.66-1.69 (2H, m), 1.95-1.99 (2H, m), 2.66 (3H, d), 3.69 (8H, s), 6.02-6.04 (1H, m), 6.73 (1H, s), 7.35 (2H, d), 7.63 (2H, d), 7.77 (2H, d), 8.71 (1H, s), 8.86 (2H, d)

### EXAMPLE 36au

¹H NMR (400.132 MHz, DMSO-d₆) δ 1.07 (3H, t), 1.66-1.69 (2H, m), 1.95-1.98 (2H, m), 3.09-3.16 (2H, m), 3.69 (8H, s), 6.13 (1H, t), 6.73 (1H, s), 7.35 (2H, d), 7.63 (2H, d), 7.77 (2H, d), 8.63 (1H, s), 8.86 (2H, d)

### EXAMPLE 36av

¹H NMR (400.132 MHz, DMSO-d₆) δ 0.40-0.44 (2H, m), 0.62-0.67 (2H, m), 1.66-1.69 (2H, m), 1.95-1.98 (2H, m), 2.53-2.57 (1H, m), 3.69 (8H, s), 6.40 (1H, d), 6.73 (1H, s), 7.36 (2H, d), 7.64 (2H, d), 7.77 (2H, d), 8.50 (1H, s), 8.86 (2H, d).

mTOR Kinase Assay (Echo): 0.00995 μM

### EXAMPLE 36aw

¹H NMR (400.132 MHz, DMSO-d₆) δ 1.66-1.69 (2H, m), 1.95-1.98 (2H, m), 2.70 (2H, t), 3.36 (2H, q), 3.69 (8H, s), 6.49 (1H, t), 6.73 (1H, s), 7.37 (2H, d), 7.64 (2H, d), 7.77 (2H, d), 8.86 (2H, d), 8.89 (1H, s)

### EXAMPLE 36ax

¹H NMR (400.132 MHz, DMSO-d₆) δ 1.56-1.63 (2H, m), 1.66-1.69 (3H, m), 1.95-1.98 (2H, m), 3.16 (2H, q), 3.47 (2H, q), 3.69 (8H, s), 4.47 (1H, t), 6.17 (1H, t), 6.73 (1H, s), 7.34 (2H, d), 7.63 (2H, d), 7.77 (2H, d), 8.86 (2H, d)

### EXAMPLE 36ay

¹H NMR (400.132 MHz, DMSO-d₆) δ 1.24-1.26 (3H, d), 1.56-1.59 (2H, q), 1.67-1.70 (2H, q), 3.19-3.26 (1H, td), 3.30 (3H, s), 3.46-3.53 (1H, td), 3.63-3.66 (1H, dd), 3.76-3.79 (1H, d), 3.97-4.00 (1H, dd), 4.22-4.25 (1H, bd), 4.58 (1H, bs), 6.81 (1H, s), 7.14 (1H, s), 7.39-7.40 (1H, d), 7.59-7.62 (2H, d), 8.28-8.30 (2H, d), 9.21 (1H, s), 10.50 (1H, s).

mTOR Kinase Assay (Echo): 0.00117 μM

### EXAMPLE 36az

¹H NMR (400.132 MHz, DMSO-d₆) δ 1.24-1.26 (3H, d), 1.56-1.59 (2H, q), 1.67-1.70 (2H, q), 2.44 (3H, s), 3.19-3.26 (1H, td), 3.31 (3H, s), 3.46-3.52 (1H, td), 3.63-3.66 (1H, dd), 3.76-3.79 (1H, d), 3.98-4.00 (1H, dd), 4.22-4.25 (1H, bd), 4.59 (1H, bs), 6.81 (1H, s), 7.61-7.63 (2H, d), 8.21-8.22 (1H, d), 8.28-8.30 (2H, d), 8.99-9.00 (1H, d), 9.41 (1H, s), 9.70 (1H, s).

mTOR Kinase Assay (Echo): 0.00149 μM

### EXAMPLE 36ba

¹H NMR (400.132 MHz, DMSO-d₆) δ 1.24-1.26 (3H, d), 1.56-1.59 (2H, q), 1.67-1.70 (2H, q), 3.19-3.25 (1H, td), 3.30 (3H, s), 3.47-3.52 (1H, td), 3.63-3.66 (1H, dd), 3.76-3.79 (1H, d), 3.97-4.00 (1H, dd), 4.22-4.25 (1H, d), 4.59 (1H, bs), 6.81 (1H, s), 7.16 (1H, s), 7.63-7.66 (2H, d), 7.84 (1H, bs), 8.28-8.30 (2H, d), 10.45 (1H, bs), 10.88 (1H, bs).

mTOR Kinase Assay (Echo): 0.00312 μM

### EXAMPLE 36bb

¹H NMR (400.132 MHz, DMSO-d₆) δ 1.23-1.24 (3H, d), 1.54-1.58 (2H, q), 1.59-1.63 (2H, m), 1.66-1.69 (2H, q), 3.15-3.25 (3H, m), 3.30 (3H, s), 3.45-3.52 (3H, m), 3.62-3.65 (1H, dd), 3.75-3.78 (1H, d), 3.96-3.99 (1H, dd), 4.20-4.23 (1H, d), 4.46-4.49 (1H, t), 4.58 (1H, bs), 6.19-6.22 (1H, t), 6.77 (1H, s), 7.49-7.51 (2H, d), 8.19-8.21 (2H, d), 8.72 (1H, s).

mTOR Kinase Assay (Echo): 0.0205 μM

### EXAMPLE 36bc

¹H NMR (400.132 MHz, DMSO-d₆) δ 0.40-0.44 (2H, m), 0.63-0.67 (2H, m), 1.16-1.18 (3H, d), 1.60-1.68 (2H, m), 1.89-1.92 (2H, m), 3.10-3.18 (1H, td), 3.42-3.49 (1H, td), 3.59-3.63 (1H, dd), 3.73-3.76 (1H, d), 3.94-3.98 (1H, dd), 4.08-4.12 (1H, d), 4.38 (1H, bs), 5.76 (1H, s), 6.40-6.41 (1H, d), 6.63 (1H, s), 7.38-7.40 (2H, d), 7.57-7.61 (2H, t), 7.70-7.74 (1H, t), 7.78-7.85 (4H, m), 8.49 (1H, s).

mTOR Kinase Assay (Echo): 0.00205 μM

### EXAMPLE 36bd

¹H NMR (400.132 MHz, DMSO-d₆) δ 1.16-1.18 (3H, d), 1.57-1.67 (4H, m), 1.81-1.92 (4H, m), 2.18-2.25 (2H, m), 3.10-3.18 (1H, td), 3.42-3.49 (1H, td), 3.59-3.62 (1H, dd), 3.73-3.76 (1H, d), 3.94-3.97 (1H, dd), 4.08-4.19 (2H, m), 4.38 (1H, bs), 6.42-6.44 (1H, d), 6.62 (1H, s), 7.35-7.37 (2H, d), 7.57-7.61 (2H, t), 7.70-7.74 (1H, tt), 7.78-7.85 (4H, m), 8.52 (1H, s).

mTOR Kinase Assay (Echo): 0.00258 μM

### EXAMPLE 36be

¹H NMR (400.132 MHz, DMSO-d₆) δ 1.16-1.18 (3H, d), 1.60-1.67 (2H, m), 1.89-1.92 (2H, m), 3.10-3.20 (1H, td), 3.30 (2H, m (under water peak)) 3.42-3.55 (2H, m), 3.59-3.63 (1H, dd), 3.73-3.76 (1H, d), 3.94-3.98 (1H, dd), 4.07-4.11 (1H, d), 4.38 (1H, bs), 6.35-6.38 (1H, t), 6.47-6.49 (1H, d), 6.63 (1H, s), 7.38-7.40 (2H, d), 7.57-7.61 (2H, t), 7.70-7.74 (1H, tt), 7.78-7.81 (2H, d), 7.84-7.86 (2H, d), 8.89 (1H, s).

mTOR Kinase Assay (Echo): 0.00376 μM

### EXAMPLE 36bf

¹H NMR (400.132 MHz, DMSO-d₆) δ 1.10-1.12 (6H, d), 1.16-1.18 (3H, d), 1.60-1.67 (2H, m), 1.59-1.92 (2H, m), 3.10-3.19 (1H, td), 3.42-3.49 (1H, td), 3.59-3.63 (1H, dd), 3.73-3.81 (2H, m), 3.94-3.97 (1H, dd), 4.09-4.12 (1H, d), 4.38 (1H, bs), 6.02-6.04 (1H, d), 6.62 (1H, s), 7.35-7.38 (2H, d), 7.57-7.61 (2H, t), 7.70-7.74 (1H, tt), 7.78-7.85 (4H, m), 8.49 (1H, s).

mTOR Kinase Assay (Echo): 0.00457 μM

### EXAMPLE 36bg

¹H NMR (400.132 MHz, DMSO-d₆) δ 1.05-1.08 (3H, t), 1.16-1.18 (3H, d), 1.60-1.67 (2H, m), 1.89-1.90 (2H, m), 3.09-3.16 (3H, m), 3.42-3.49 (1H, m), 3.59-3.63 (1H, dd), 3.73-3.76 (1H, d), 3.95-3.97 (1H, dd), 4.09-4.12 (1H, d), 4.38 (1H, bs), 6.12-6.15 (1H, t), 6.62 (1H, s), 7.37-7.39 (2H, d), 7.57-7.61 (2H, t), 7.70-7.74 (1H, t), 7.78-7.85 (4H, m), 8.62 (1H, s).

mTOR Kinase Assay (Echo): 0.000922 μM

### EXAMPLE 36bh

¹H NMR (400.132 MHz, DMSO-d₆) δ 1.16-1.18 (3H, d), 1.60-1.67 (2H, m), 1.89-1.92 (2H, m), 3.10-3.15 (1H, td), 3.17-3.19 (2H, m), 3.42-3.49 (3H, m), 3.59-3.63 (1H, dd), 3.73-3.76 (1H, d), 3.94-3.98 (1H, dd), 4.08-4.11 (1H, d), 4.38 (1H, bs), 4.71-4.74 (1H, t), 6.22-6.24 (1H, t), 6.62 (1H, s), 7.36-7.39 (2H, d), 7.57-7.61 (2H, t), 7.70-7.74 (1H, tt), 7.78-7.85 (4H, m), 8.76 (1H, s).

mTOR Kinase Assay (Echo): 0.00121 μM

### EXAMPLE 36bi

¹H NMR (400.132 MHz, DMSO-d₆) δ 0.87-0.91 (3H, t), 1.16-1.18 (3H, d), 1.41-1.50 (2H, m), 1.60-1.67 (2H, m), 1.89-1.92 (2H, m), 3.04-3.09 (2H, q), 3.10-3.17 (1H, 10 td), 3.42-3.49 (1H, td), 3.59-3.63 (1H, dd), 3.73-3.76 (1H, d), 3.94-3.97 (1H, dd), 4.08-4.12 (1H, d), 4.39 (1H, bs), 6.16-6.19 (1H, t), 6.62 (1H, s), 7.37-7.39 (2H, d), 7.57-7.61 (2H, t), 7.70-7.74 (1H, tt), 7.78-7.85 (4H, m), 8.61 (1H, s).

mTOR Kinase Assay (Echo): 0.00231 μM

### EXAMPLE 36bj

¹H NMR (400.132 MHz, DMSO-d₆) δ 1.16-1.18 (3H, d), 1.60-1.68 (2H, m), 1.89-1.92 (2H, m), 2.65-2.66 (3H, d), 3.10-3.17 (1H, td), 3.42-3.49 (1H, td), 3.59-3.63 (1H, dd), 3.73-3.76 (1H, d), 3.94-3.97 (1H, dd), 4.09-4.12 (1H, d), 4.38 (1H, bs), 6.03-6.06 (1H, q), 6.62 (1H, s), 7.38-7.40 (2H, d), 7.57-7.61 (2H, t), 7.70-7.73 (1H, tt), 7.74-7.85 (4H, m), 8.70 (1H, s).

mTOR Kinase Assay (Echo): 0.000621 μM

### EXAMPLE 36bk

¹H NMR (400.132 MHz, DMSO-d₆) δ 1.16-1.18 (3H, d), 1.24 (6H, s), 1.60-1.67 (2H, m), 1.88-1.91 (2H, m), 3.10-3.17 (1H, td), 3.38-3.40 (2H, d), 3.42-3.49 (1H, td), 3.59-3.63 (1H, dd), 3.73-3.76 (1H, d), 3.94-3.98 (1H, dd), 4.08-4.12 (1H, d), 4.38 (1H, bs), 4.93-4.96 (1H, t), 5.98 (1H, s), 6.62 (1H, s), 7.33-7.35 (2H, d), 7.58-7.61 (2H, t), 7.70-7.74 (1H, m), 7.78-7.84 (4H, m), 8.70 (1H, s).

mTOR Kinase Assay (Echo): 0.00433 μM

### EXAMPLE 36bl

¹H NMR (400.132 MHz, DMSO-d₆) δ 1.16-1.18 (3H, d), 1.56-1.67 (4H, m), 1.89-1.92 (2H, m), 3.10-3.17 (3H, m), 3.42-3.49 (3H, m), 3.59-3.63 (1H, dd), 3.73-3.76 (1H, d), 3.94-3.97 (1H, dd), 4.08-4.12 (1H, d), 4.38 (1H, bs), 4.46-4.49 (1H, t), 6.16-6.19 (1H, t), 6.62 (1H, s), 7.37-7.39 (2H, d), 7.57-7.61 (2H, t), 7.70-7.74 (1H, tt), 7.78-7.85 (4H, m), 8.67 (1H, s).

mTOR Kinase Assay (Echo): 0.00208 μM

### EXAMPLE 36bm

¹H NMR (400.132 MHz, DMSO-d₆) δ 1.17-1.18 (3H, d), 1.60-1.68 (2H, m), 1.89-1.92 (2H, m), 2.68-2.72 (2H, t), 3.11-3.18 (1H, td), 3.34-3.39 (2H, q), 3.43-3.49 (1H, td), 3.59-3.63 (1H, dd), 3.73-3.76 (1H, d), 3.94-3.97 (1H, dd), 4.09-4.12 (1H, d), 4.38 (1H, bs), 6.49-6.52 (1H, t), 6.63 (1H, s), 7.39-7.41 (2H, d), 7.57-7.61 (2H, t), 7.70-7.74 (1H, tt), 7.78-7.86 (4H, m), 8.88 (1H, s).

mTOR Kinase Assay (Echo): 0.00193 μM

### EXAMPLE 36bn

¹H NMR (400.132 MHz, DMSO-d₆) δ 1.16-1.18 (3H, d), 1.60-1.68 (2H, m), 1.88-1.93 (2H, m), 3.10-3.17 (1H, td), 3.42-3.49 (1H, td), 3.59-3.63 (1H, dd), 3.73-3.76 (1H, d), 3.94-3.97 (1H, dd), 4.08-4.12 (1H, d), 4.31-4.33 (2H, d), 4.38 (1H, bs), 6.59-6.63 (2H, m), 6.94 (2H, bs), 7.39-7.41 (2H, d), 7.57-7.61 (2H, t), 7.70-7.74 (1H, t), 7.78-7.81 (2H, d), 7.84-7.87 (2H, d), 8.88 (1H, s), 11.84 (1H, bs).

mTOR Kinase Assay (Echo): 0.00637 μM

### EXAMPLE 36bo

¹H NMR (400.132 MHz, DMSO-d₆) δ 0.63-0.66 (2H, q), 0.69-0.73 (2H, q), 1.16-1.18 (3H, d), 1.60-1.67 (2H, m), 1.89-1.92 (2H, m), 3.10-3.17 (1H, td), 3.42-3.49 (3H, m), 3.59-3.63 (1H, dd), 3.73-3.76 (1H, d), 3.94-3.98 (1H, dd), 4.09-4.12 (1H, d), 4.38 (1H, bs), 4.83 (1H, bs), 6.54 (1H, s), 6.63 (1H, s), 7.35-7.37 (2H, d), 7.57-7.61 (2H, t), 7.70-7.74 (1H, tt), 7.74-7.85 (4H, m), 8.63 (1H, s).

mTOR Kinase Assay (Echo): 0.0176 μM

### EXAMPLE 36bp

¹H NMR (400.132 MHz, DMSO-d₆) δ 1.16-1.18 (3H, d), 1.60-1.67 (2H, m), 1.89-1.90 (2H, m), 3.10-3.17 (1H, td), 3.42-3.49 (1H, td), 3.59-3.62 (1H, dd), 3.73-3.76 (1H, d), 3.94-3.97 (1H, dd), 4.08-4.12 (1H, d), 4.38 (1H, bs), 4.43-4.46 (2H, t), 4.72-4.81 (3H, m), 6.63 (1H, s), 6.91-6.93 (1H, d), 7.37-7.39 (2H, d), 7.57-7.61 (2H, t), 7.70-7.74 (1H, tt), 7.78-7.86 (4H, m), 8.73 (1H, s).

mTOR Kinase Assay (Echo): 0.00198 μM

### EXAMPLE 36bq

¹H NMR (400.132 MHz, DMSO-d₆) δ 1.18-1.19 (3H, d), 1.61-1.66 (2H, m), 1.91-1.92 (2H, m), 3.12-3.18 (1H, td), 3.44-3.50 (1H, td), 3.61-3.64 (1H, dd), 3.74-3.77 (1H, d), 3.95-3.99 (1H, dd), 4.10-4.14 (1H, d), 4.40 (1H, bs), 6.66 (1H, s), 7.52-7.54 (2H, d), 7.59-7.62 (2H, t), 7.72-7.76 (1H, t), 7.79-7.81 (3H, d), 7.90-7.92 (2H, d), 8.28 (1H, s), 9.39 (1H, s).

mTOR Kinase Assay (Echo): 0.00131 μM

### EXAMPLE 36br

¹H NMR (400.132 MHz, DMSO-d₆) δ 1.17-1.18 (3H, d), 1.61-1.68 (2H, m), 1.90-1.91 (2H, m), 3.11-3.17 (1H, td), 3.43-3.50 (1H, td), 3.59-3.63 (1H, dd), 3.74-3.76 (1H, d), 3.79 (3H, s), 3.94-3.98 (1H, dd), 4.09-4.13 (1H, d), 4.39 (1H, bs), 6.64 (1H, s), 7.38 (1H, s), 7.42-7.44 (2H, d), 7.58-7.62 (2H, t), 7.71-7.73 (1H, t), 7.77-7.81 (3H, m), 7.86-7.88 (2H, d), 8.36 (1H, s), 8.80 (1H, s).

mTOR Kinase Assay (Echo): 0.00188 μM

### EXAMPLE 36bs

¹H NMR (400.132 MHz, DMSO-d₆) δ 0.39-0.43 (2H, m), 0.62-0.67 (2H, m), 1.19-1.20 (3H, d), 1.82-1.93 (1H, m), 2.03-2.14 (1H, m), 2.54 (1H, m), 2.75-2.82 (2H, m), 3.01-3.09 (2H, m), 3.10-3.17 (1H, td), 3.44-3.51 (1H, td), 3.61-3.65 (1H, dd), 3.74-3.77 (1H, d), 3.94-3.98 (1H, dd), 4.06-4.09 (1H, d), 4.44 (1H, bs), 6.40-6.41 (1H, d), 6.46 (1H, s), 7.36-7.39 (2H, d), 7.43-7.51 (4H, m), 7.58-7.62 (1H, m), 7.78-7.80 (2H, d), 8.48 (1H, s).

mTOR Kinase Assay (Echo): 0.00161 μM

### EXAMPLE 36bt

¹H NMR (400.132 MHz, DMSO-d₆) δ 1.19-1.21 (3H, d), 1.82-1.93 (1H, m), 2.03-2.14 (1H, m), 2.74-2.82 (2H, m), 3.02-3.10 (2H, m), 3.13-3.19 (3H, m), 3.44-3.51 (3H, m), 3.62-3.65 (1H, dd), 3.74-3.77 (1H, d), 3.94-3.98 (1H, dd), 4.07-4.10 (1H, d), 4.45 (1H, bs), 4.71-4.74 (1H, t), 6.22-6.25 (1H, t), 6.47 (1H, s), 7.35-7.37 (2H, d), 7.44-7.51 (4H, m), 7.58-7.62 (1H, m), 7.78-7.81 (2H, d), 8.74 (1H, s).

mTOR Kinase Assay (Echo): 0.00105 μM

### EXAMPLE 36bu

¹H NMR (400.132 MHz, DMSO-d₆) δ 1.19-1.21 (3H, d), 1.82-1.93 (1H, m), 2.04-2.14 (1H, m), 2.65-2.66 (3H, d), 2.75-2.82 (2H, m), 3.02-3.11 (2H, m), 3.13-3.19 (1H, td), 3.45-3.51 (1H, td), 3.62-3.65 (1H, dd), 3.74-3.77 (1H, d), 3.94-3.98 (1H, dd), 4.06-4.10 (1H, d), 4.45 (1H, bs), 6.03-6.06 (1H, q), 6.46 (1H, s), 7.36-7.38 (2H, d), 7.44-7.51 (4H, m), 7.58-7.62 (1H, m), 7.78-7.80 (2H, d), 8.68 (1H, s).

mTOR Kinase Assay (Echo): 0.00118 μM

### EXAMPLE 36bv

¹H NMR (400.132 MHz, DMSO-d₆) δ 1.20-1.21 (3H, d), 1.83-1.94 (1H, m), 2.04-2.15 (1H, m), 2.76-2.83 (2H, m), 3.02-3.10 (2H, m), 3.11-3.18 (1H, td), 3.45-3.52 (1H, td), 3.62-3.66 (1H, dd), 3.75-3.77 (1H, d), 3.79 (3H, s), 3.95-3.98 (1H, dd), 4.07-4.10 (1H, d), 4.46 (1H, bs), 6.48 (1H, s), 7.38-7.51 (7H, m), 7.59-7.76 (1H, m), 7.76 (1H, s), 7.82-7.84 (2H, d), 8.36 (1H, s), 8.78 (1H, s).

mTOR Kinase Assay (Echo): 0.00264 μM

### EXAMPLE 36bw

¹H NMR (400.132 MHz, DMSO-d₆) δ 0.40-0.44 (2H, m), 0.63-0.67 (2H, m), 1.20-1.22 (3H, d), 1.49-1.58 (2H, m), 1.80-1.87 (2H, m), 2.53 (2H, m), 2.67-2.73 (2H, m), 3.12-3.18 (1H, td), 3.46-3.53 (1H, td), 3.63-3.67 (1H, dd), 3.75-3.78 (1H, d), 3.95-3.99 (1H, dd), 4.09-4.12 (1H, d), 4.48-4.49 (1H, bs), 6.40-6.41 (1H, d), 6.62 (1H, s), 7.37-7.39 (2H, d), 7.42-7.48 (4H, m), 7.57-7.61 (1H, m), 7.80-7.82 (2H, d), 8.48 (1H, s).

mTOR Kinase Assay (Echo): 0.00909 μM

### EXAMPLE 36bx

¹H NMR (400.132 MHz, DMSO-d₆) δ 1.20-1.22 (3H, d), 1.50-1.59 (2H, m), 1.80-1.89 (2H, m), 2.52-2.61 (2H, m), 2.66-2.74 (2H, m), 3.11-3.19 (3H, m), 3.44-3.52 (3H, m), 3.63-3.67 (1H, dd), 3.75-3.78 (1H, d), 3.95-3.99 (1H, dd), 4.08-4.12 (1H, d), 4.48 (1H, bs), 4.71-4.74 (1H, t), 6.22-6.25 (1H, t), 6.62 (1H, s), 7.35-7.38 (2H, d), 7.42-7.48 (4H, m), 7.57-7.61 (1H, m), 7.80-7.82 (2H, d), 8.74 (1H, s).

mTOR Kinase Assay (Echo): 0.00269 μM

### EXAMPLE 36by

¹H NMR (400.132 MHz, DMSO-d₆) δ 1.20-1.22 (3H, d), 1.52-1.56 (2H, m), 1.57-1.63 (2H, m), 1.79-1.88 (2H, m), 2.54-2.61 (2H, m), 2.67-2.72 (2H, m), 3.11-3.17 (3H, m), 3.45-3.52 (3H, m), 3.63-3.66 (1H, dd), 3.75-3.78 (1H, d), 3.95-3.99 (1H, dd), 4.09-4.12 (1H, d), 4.46-4.49 (2H, m), 6.17-6.19 (1H, t), 6.62 (1H, s), 7.35-7.38 (2H, d), 7.42-7.48 (4H, m), 7.57-7.61 (1H, m), 7.79-7.82 (2H, d), 8.65 (1H, s).

mTOR Kinase Assay (Echo): 0.012 μM

### EXAMPLE 36bz

¹H NMR (400.132 MHz, DMSO-d₆) δ 1.21-1.22 (3H, d), 1.50-1.59 (2H, m), 1.81-1.88 (2H, m), 2.53-2.59 (2H, m), 2.67-2.72 (4H, m), 3.11-3.19 (1H, td), 3.34-3.39 (2H, m), 3.46-3.52 (1H, td), 3.63-3.67 (1H, dd), 3.75-3.78 (1H, d), 3.95-3.99 (1H, dd), 4.09-4.13 (1H, d), 4.49 (1H, bs), 6.49-6.52 (1H, t), 6.63 (1H, s), 7.38-7.40 (2H, d), 7.42-7.48 (4H, m), 7.57-7.61 (1H, m), 7.81-7.83 (2H, d), 8.86 (1H, s).

mTOR Kinase Assay (Echo): 0.0143 μM

### EXAMPLE 36ca

¹H NMR (400.132 MHz, DMSO-d₆) δ 1.23-1.25 (3H, d), 1.68-1.74 (2H, m), 2.20-2.27 (2H, td), 2.67-2.68 (3H, d), 2.82-2.87 (2H, t), 3.05-3.09 (2H, m), 3.18-3.22 (1H, m), 3.22-3.25 (2H, t), 3.37-3.40 (2H, t), 3.50-3.58 (1H, td), 3.66-3.70 (1H, dd), 3.77-3.80 (1H, d), 3.90-3.96 (2H, m), 3.98-4.01 (1H, dd), 4.39-4.32 (1H, d), 4.57 (1H, s), 6.10-6.13 (1H, q), 6.86 (1H, s), 7.50-7.53 (2H, d), 8.21-8.23 (2H, d), 8.79 (1H, s)

### EXAMPLE 36cb

¹H NMR (400.132 MHz, DMSO-d₆) δ 1.23-1.25 (3H, d), 1.68-1.75 (2H, m), 2.20-2.27 (2H, td), 2.54 (3H, d), 2.82-2.87 (2H, t), 3.06-3.11 (2H, m), 3.12-3.16 (1H, m), 3.18-3.24 (2H, m), 3.37-3.40 (2H, t), 3.50-3.57 (1H, td), 3.66-3.70 (1H, dd), 3.77-3.80 (1H, d), 3.91-3.95 (2H, m), 4.00-4.02 (1H, dd), 4.29-4.32 (1H, d), 4.58 (1H, s), 6.27-6.29 (1H, t), 6.86 (1H, s), 7.50-7.52v(2H, d), 8.21-8.23 (2H, d), 8.78 (1H, s)

### EXAMPLE 36cc

¹H NMR (400.132 MHz, DMSO-d₆) δ 1.23-1.25 (2H, d), 1.68-1.75 (2H, m), 2.20-2.27 (2H, td), 2.538-2.543 (3H, d), 2.82-2.87 (2H, t), 3.06-3.09 (2H, m), 3.16-3.21 (1H, m), 3.22-3.25 (2H, m), 3.37-3.40 (1H, td), 3.66-3.70 (1H, dd), 3.77-3.80 (1H, d), 3.90-3.96 (2H, m), 4.00-4.02 (1H, dd), 4.29-4.33 (1H, d), 4.58 (1H, s), 6.49-6.50 (1H, d), 6.86 (1H, s), 7.51-7.53 (2H, d), 8.22-8.24 (2H, d), 8.61 (1H, s)

### EXAMPLE 36cd

¹H NMR (400.132 MHz, DMSO-d₆) δ 1.23-1.25 (3H, d), 1.55-1.65 (2H, m), 1.67-1.74 (2H, m), 2.19-2.26 (4H, m), 2.537-2.541 (3H, d), 2.81-2.86 (2H, t), 3.05-3.09 (2H, m), 3.16-3.22 (2H, m), 3.24-3.26 (1H, m), 3.37-3.40 (2H, t), 3.49-3.56 (1H, td), 3.66-3.70 (1H, dd), 3.77-3.79 (1H, d), 3.90-3.96 (2H, m), 3.98-4.01 (1H, dd), 4.11-4.21 (1H, sex), 4.29-4.32 (1H, d), 4.57 (1H, s), 6.52-6.54 (1H, d), 6.86 (1H, s), 7.48-7.50 (2H, d), 7.54-7.67(1H, m), 8.21-8.23 (2H, d), 8.63 (1H, s)

### EXAMPLE 36ce

¹H NMR (400.132 MHz, DMSO-d₆) δ 1.23-1.35 (3H, d), 1.67-1.74 (2H, m), 2.20-2.27 (2H, td), 2.538-2.541 (3H, d), 2.70-3.73 (2H, t), 2.82-2.87 (2H, t), 3.05-3.09 (2H, m), 3.18-3.23 (2H, m), 3.25-3.26 (1H, m), 3.18-3.23 (2H, m), 2.25-2.26 (1H, m), 3.36-3.40 (4H, m), 3.50-3.56 (1H, td), 3.66-3.70 (1H, dd), 3.77-3.80 (1H, d), 3.90-3.96 (2H, qu), 3.98-4.02 (1H, dd), 4.30-4.33 (1H, d), 4.56-4.58 (1H, m), 6.54-6.57 (1H, t), 6.86 (1H, s), 7.52-7.54 (2H, d), 8.23-8.25 (2H, d), 8.95 (1H, s)

### EXAMPLE 36cf

¹H NMR (400.132 MHz, DMSO-d₆) δ 1.23-1.25 (3H, d), 1.68-1.75 (2H, m), 2.20-2.27 (2H, td), 2.82-2.87 (2H, t), 3.16-3.22 (2H, m), 3.25 (1H, m), 3.37-3.40 (2H, t), 3.50-3.56 (1H, td), 3.66-3.70 (1H, dd), 3.77-3.80 (1H, d), 3.90-3.96 (2H, m), 4.00-4.02 (1H, dd), 4.29-4.33 (1H, d), 4.58 (1H, s), 6.23-6.25 (1H, t), 6.86 (1H, t), 7.49-7.52 (2H, d), 8.21-8.23 (2H, d), 8.69 (1H, s)

### EXAMPLE 36cg

¹H NMR (400.132 MHz, DMSO-d₆) δ 1.23-1.25 (3H, d), 1.68-1.75 (2H, m), 2.20-2.27 (2H, td), 2.82-2.87 (2H, t), 3.06-3.09 (2H, m), 3.17-3.23 (4H, m), 3.25-3.26 (1H, m), 3.37-3.40(2H, t), 3.46-3.48 (2H, t), 3.50-3.56 (1H, td), 3.66-3.70 (1H, dd), 3.77-3.80 (1H, d), 3.90-3.96 (2H, qu), 3.98-4.02 (1H, dd) 4.29-4.33 (1H, d), 4.57 (1H, s), 6.30-6.33 (1H, t), 6.86 (1H, s), 7.49-7.51 (2H, d), 8.22-8.24 (2H, d), 8.87 (1H, s)

### EXAMPLE 36ch

¹H NMR (400.132 MHz, DMSO-d₆) δ 1.24-1.26 (3H, d), 1.68-1.75 (2H, m), 2.20-2.28 (2H, td), 2.82-2.87 (2H, t), 3.06-3.10 (2H, m), 3.18-3.22 (2H, m), 3.23-3.25 (1H, m), 3.38-3.41 (2H, t), 3.50-3.57 (1H, td), 3.67-3.70 (1H, dd), 3.77 (1H, s), 3.80 (3H, s), 3.91-3.96 (2H, qu), 3.98-4.02 (1H, dd), 4.30-4.33 (1H, d), 4.58 (1H, exchange), 6.87 (1H, s), 7.390-7.392 (1H, d), 7.55-7.57 (2H, d), 7.77 (1H, s), 8.25-8.27 (2H, d), 8.49 (1H, exchange), 8.93 (1H, exchange)

### EXAMPLE 36ci

¹H NMR (400.132 MHz, DMSO-d₆) δ 1.24-1.25 (3H, d), 1.68-1.75 (2H, m), 2.20-2.28 (2H, td), 2.82-2.86 (2H, t), 3.07-3.11 (2H, m), 3.16-3.20 (1H, m), 3.22-3.23 (1H, d), 3.38-3.41 (2H, t), 3.51-3.57 (2H, td), 3.68-3.69 (1H, dd), 3.77-3.80 (1H, d), 3.90-3.96 (2H, t), 3.99-4.01 (1H, d), 4.29-4.33 (1H, d), 4.57-4.58 (1H, exchange), 6.84 (1H, s), 7.23 (1H, s), 7.57-7.59 (1H, m), 7.65-7.67 (2H, d), 8.20-8.22 (2H, d)

### EXAMPLE 36cj

¹H NMR (400.132 MHz, DMSO-d₆) δ 1.24-1.26 (3H, d), 1.63-1.66 (2H, m), 1.93-2.00 (2H, m), 3.15-3.20 (2H, m), 3.217-3.226 (1H, d), 3.47-3.51 (1H, dd), 3.53-3.57 (4H, m), 3.63-3.67 (1H, dd), 3.76-3.79 (1H, d), 3.97-4.01 (1H, dd), 4.22-4.25 (1H, d), 4.57 (1H, exchange), 4.73 (1H, exchange), 6.77 (1H, s), 6.84-6.85 (1H, d), 7.33-7.34 (1H, d), 7.66-7.68 (2H, d), 8.23-8.25 (2H, d)

### EXAMPLE 36ck

¹H NMR (400.132 MHz, DMSO-d₆) δ 0.70-0.74 (2H, m), 0.91-0.93 (2H, dd), 1.24-1.26 (3H, d), 1.56-1.58 (2H, m), 1.64-1.66 (2H, m), 1.92-1.99 (2H, m), 2.72-2.77 (1H, sep), 2.89 (2H, s), 3.16-3.20 (2H, t), 3.22-3.23 (1H, d), 3.4703.50 (1H, dd), 3.51-3.56 (4H, m), 3.63-3.67 (1H, dd), 3.77-3.80 (1H, d), 3.97-4.01 (1H, dd), 4.22-4.25 (1H, d), 4.57 (1H, exchange), 6.79 (1H, s), 7.64-7.67 (2H, dd), 8.22-8.24 (2H, s), 8.41 (1H, exchange)

### EXAMPLE 36cl

¹H NMR (400.132 MHz, DMSO-d₆) δ 0.56-0.59 (2H, t), 0.66-0.68 (2H, t), 1.24-1.26 (3H, d), 1.31-1.36 (3H, m), 1.56-1.60 (2H, m), 1.62-1.66 (2H, m), 1.92-2.00 (2H, m), 3.16-3.21 (2H, t), 3.47-3.50 (1H, dd), 2.52-3.55 (4H, m), 3.63-3.67 (1H, dd), 3.52-3.55 (4H, m), 3.63-3.67 (1H, dd), 3.77-3.79 (1H, d), 4.00-4.01 (1H, dd), 4.21-4.24 (1H, d), 4.56 (1H, exchange), 6.78 (1H, s), 7.49-7.51 (2H, d), 8.21-8.23 (2H, d)

### EXAMPLE 36cm

¹H NMR (400.132 MHz, DMSO-d₆) δ 1.24-1.26 (3H, d), 1.56-1.58 (2H, m), 1.64-1.67 (2H, m), 1.92-1.99 (2H, m), 3.19-3.26 (1H, dd), 3.51-3.55 (6H, m), 3.63-3.67 (1H, dd), 3.77-3.79 (1H, d), 3.97-4.01 (1H, dd), 4.21-4.24 (1H, d), 4.57 (1H, s), 4.71 (1H, s), 5.94-6.24 (1H, tt), 6.55-6.58 (1H, t), 6.80 (1H, s), 7.51-7.54 (2H, d), 8.23-8.26 (2H, d), 8.96 (1H, s)

### EXAMPLE 36cn

¹H NMR (400.132 MHz, DMSO-d₆) δ 1.24-1.26 (3H, d), 1.32 (9H, s), 1.54-1.60 (2H, m), 1.61-1.67 (2H, m), 1.92-1.99 (2H, m), 3.18-3.26 (1H, m), 3.47-3.50 (1H, dd), 3.52-3.56 (4H, m), 3.63-3.67 (1H, dd), 3.77-3.80 (1H, d), 3.97-4.01 (1H, dd), 4.21-4.24 (1H, d), 4.57 (1H, s), 4.72 (1H, exchange), 6.09 (1H, s), 6.78 (1H, s), 7.45-7.48 (2H, d), 8.20-8.23 (2H, d), 8.52 (1H, s)

### EXAMPLE 36co

¹H NMR (400.132 MHz, DMSO-d₆) δ 1.23-1.25 (3H, d), 1.55-1.59 (2H, m), 1.60-1.64 (2H, m), 1.91-1.98 (2H, m), 3.46-3.50 (1H, dd), 3.50-3.54 (4H, m), 3.62-3.66 (1H, dd), 3.75-3.78 (1H, d), 3.96-3.99 (1H, dd), 4.08 (1H, s), 4.19-4.22 (1H, d), 4.55 (exchange), 4.70 (exchange), 6.73 (1H, s), 7.59-7.61 (2H, d), 8.10-8.12 (2H, d), 8.38 (1H, exchange)

### EXAMPLE 36cp

¹H NMR (400.132 MHz, DMSO-d₆) δ 1.24-1.25 (3H, d), 1.55-1.59 (2H, m), 1.62-1.66 (2H, m), 1.77-1.84 (2H, m), 1.91-1.99 (2H, m), 3.16-3.20 (2H, m), 3.21-3.25 (1H, dd), 3.46-3.47 (1H, d), 3.49-3.55 (6H, m), 3.62-3.66 (1H, dd), 3.76-3.79 (1H, d), 3.96-4.00 (1H, dd), 4.21-4.25 (1H, d), 4.31-4.32 (1H, m), 4.56 (exchange), 6.78 (1H, s), 7.64-7.66 (2H, d), 8.20-8.22 (2H, d), 8.34 (exchange)

### EXAMPLE 36cq

¹H NMR (400.132 MHz, DMSO-d₆) δ 1.23-1.25 (3H, d), 1.55-1.59 (2H, m), 1.62-1.65 (2H, m), 1.91-1.98 (2H, m), 3.05 (3H, s), 3.16-3.25 (2H, m), 3.33-3.34 (2H, d), 3.46-3.50 (1H, dd), 3.50-3.58 (6H, m), 3.62-3.66 (1H, dd), 3.76-3.79 (1H, d), 3.96-4.00 (1H, dd), 4.20-4.23 (1H, d), 4.56 (exchange), 4.70 (exchange), 6.45-6.48 (1H, t), 6.78 (1H, s), 7.51-7.53 (2H, d), 8.21-8.24 (2H, d), 9.05 (exchange)

### EXAMPLE 36cr

¹H NMR (400.132 MHz, DMSO-d₆) δ 1.24-1.26 (3H, d), 1.56-1.60 (2H, m), 1.62-1.66 (2H, m), 1.92-1.99 (2H, m), 3.00-3.05 (1H, dd), 3.16-3.22 (6H, m), 3.42-3.47 (2H, dd), 3.51-3.55 (4H, m), 3.63-3.67 (1H, dd), 3.76-3.79 (1H, d), 3.97-4.00 (1H, dd), 4.21-4.24 (1H, d), 4.45-4.48 (exchange, t), 4.56 (exchange), 6.79 (1H, s), 7.11 (exchange), 7.52-7.54 (2H, d), 8.22-8.24 (2H, d), 9.21 (exchange)

### EXAMPLE 36cs

¹H NMR (400.132 MHz, DMSO-d₆) δ 1.24-1.25 (3H, d), 1.56-1.58 (2H, m), 1.62-1.66 (2H, m), 1.91-1.98 (2H, m), 2.89 (3H, s), 2.98 (3H, s), 3.16-3.22 (2H, m), 3.46-3.50 (1H, dd), 3.50-2.54 (4H, m), 3.63-3.66 (1H, dd), 3.76-3.79 (1H, d), 3.96-3.97 (1H, d), 3.99-4.00 (2H, d), 4.20-4.24 (1H, d), 4.57 (1H, exchange), 6.43-6.45 (t, exchange), 6.79 (1H, s), 7.50-7.52 (2H, d), 8.22-8.24 (2H, d), 9.19 (exchange)

### EXAMPLE 36ct

¹H NMR (400.132 MHz, DMSO-d₆) δ 1.15-1.20 (3H, d), 1.70-1.79 (2H, q), 1.95-2.05 (2H, q), 3.10-3.20 (1H, td), 3.45-3.52 (1H, td), 3.62-3.65 (1H, dd), 3.75-3.78 (1H, d), 3.96-3.99 (1H, dd), 4.10-4.20 (1H, d), 4.45 (1H, bs), 6.70 (1H, s), 7.45-7.52 (2H, d), 7.75-7.80 (1H, dd), 7.80-7.82 (2H, d), 8.00-8.05 (1H, d), 8.10-8.15 (1H, td), 8.40 (1H, s), 8.85 (1H, d), 9.40 (1H, s), 11.40 (1H, br s).

mTOR Kinase Assay (Echo): 0.00157 μM

### EXAMPLE 36cu

¹H NMR (400.132 MHz, DMSO-d₆) δ 1.10-1.15 (3H, d), 1.70-1.79 (2H, q), 1.95-2.00 (2H, q), 3.10-3.20 (1H, td), 3.45-3.50 (1H, td), 3.60-3.62 (1H, dd), 3.75-3.78 (1H, d), 3.96-3.99 (1H, dd), 4.10-4.20 (1H, d), 4.30 (2H, d), 4.45 (1H, bs), 6.60 (1H, t), 6.70 (1H, s), 6.90-7.00 (2H, br d), 7.40 (2H, d), 7.75-7.80 (3H, m), 8.00-8.05 (1H, d), 8.10-8.15 (1H, td), 8.80 (1H, d), 8.90 (1H, s).

mTOR Kinase Assay (Echo): 0.0686 μM

### EXAMPLE 36cv

¹H NMR (400.132 MHz, DMSO-d₆) δ 0.55-0.65 (2H, q), 0.65-0.75 (2H, q), 1.10-1.15 (3H, d), 1.70-1.79 (2H, q), 1.95-2.00 (2H, q), 3.10-3.20 (1H, td), 3.40-3.50 (2H, m), 3.60-3.62 (1H, dd), 3.75-3.78 (1H, d), 3.96-3.99 (1H, dd), 4.10-4.20 (1H, d), 4.45 (1H, bs), 4.80 (1H, bs), 6.55 (1H, t), 6.65 (1H, s), 7.30 (2H, d), 7.70-7.80 (3H, m), 7.95-8.00 (1H, d), 8.05-8.10 (1H, td), 8.60 (1H, s), 8.80 (1H, d).

mTOR Kinase Assay (Echo): 0.259 μM

### EXAMPLE 36cw

¹H NMR (400.132 MHz, DMSO-d₆) δ 1.15-1.20 (3H, d), 1.65-1.70 (2H, q), 1.95-2.00 (2H, q), 3.10-3.20 (1H, td), 3.40-3.50 (1H, td), 3.60-3.64 (1H, dd), 3.75-3.78 (1H, d), 3.96-3.99 (1H, dd), 4.10-4.20 (1H, d), 4.35 (2H, d), 4.45 (1H, bs), 6.60 (1H, t), 6.70 (1H, s), 6.80-7.10 (2H, m), 7.40 (2H, d), 7.70 (2H, d), 7.80 (2H, dd), 8.85 (2H, dd), 8.90 (1H, s), 11.85 (1H, s).

### EXAMPLE 36cx

¹H NMR (400.132 MHz, DMSO-d₆) δ 0.55-0.65 (2H, q), 0.65-0.75 (2H, q), 1.15-1.20 (3H, d), 1.70-1.75 (2H, q), 1.95-2.00 (2H, q), 3.10-3.20 (1H, td), 3.45 (2H, d), 3.50 (1H, td), 3.60-3.63 (1H, dd), 3.74-3.78 (1H, d), 3.94-3.99 (1H, dd), 4.10-4.20 (1H, d), 4.45 (1H, bs), 4.80-4.90 (1H, t), 6.55 (1H, s), 6.65 (1H, s), 7.30 (2H, d), 7.65 (2H, d), 7.80 (2H, dd), 8.70 (1H, s), 8.85 (2H, dd)

### EXAMPLE 36cy

¹H NMR (400.132 MHz, DMSO-d₆) δ 1.21 (3H, m), 2.22-2.30 (2H, td), 2.65 (3H, d), 2.75 (2H, t), 3.13 (3H, m), 3.50 (1H, td), 3.65 (1H, dd), 3.75 (1H, d), 3.90-3.95 (2H, d), 3.98 (1H, dd), 4.15 (1H, d), 4.50 (1H, br s), 6.05 (1H, q), 6.68 (1H, s), 7.37 (2H, d), 7.44 (2H, d), 7.46 (2H, d), 7.61 (1H, tt), 7.76 (2H, d), 8.68 (1H, s)

### EXAMPLE 36cz

¹H NMR (400.132 MHz, DMSO-d₆) δ 1.06 (3H, t), 1.21 (3H, d), 2.26-2.31 (2H, td), 2.74 (2H, t), 3.12 (5H, dt), 3.50 (1H, td), 3.65 (1H, dd), 3.76 (1H, d), 3.90 (2H, d), 3.97 (1H, dd), 4.14 (1H, d), 4.52 (1H, m), 6.14 (1H, t), 6.68 (1H, s), 7.36 (2H, d), 7.44 (4H, q), 7.60 (1H, m), 7.76 (2H, d), 8.60 (1H, s)

### EXAMPLE 36da

¹H NMR (400.132 MHz, DMSO-d₆) δ 0.41 (2H, q), 0.65 (2H, q), 1.21 (3H, d), 2.23-2.32 (2H, td), 2.55 (1H, dd), 2.75 (2H, m), 3.15 (3H, m), 3.50 (1H, td), 3.66 (1H, dd), 3.76 (1H, d), 3.91 (2H, d), 3.97 (1H, dd), 4.14 (1H, d), 4.51 (1H, br s), 6.41 (1H, d), 6.69 (1H, s), 7.37 (2H, d), 7.44 (4H, dd), 7.61 (1H, t), 7.77 (2H, d), 8.48 (1H, s)

### EXAMPLE 36db

¹H NMR (400.132 MHz, DMSO-d₆) δ 1.21 (3H, d), 2.26-2.31 (2H, td), 2.74-2.78 (2H, t), 3.11-3.17 (5H, m), 3.46 (2H, q), 3.56 (1H, dd), 3.66 (1H, dd), 3.76 (1H, d), 3.90 (2H, d), 3.97 (1H, dd), 4.15 (1H, d), 4.50 (1H, br s), 4.72 (1H, t), 6.24 (1H, t), 6.68 (1H, s), 7.36 (2H, d), 7.44 (4H, q), 7.60 (1H, m), 7.77 (2H, d), 8.75 (1H, s)

### EXAMPLE 36dc

¹H NMR (400.132 MHz, DMSO-d₆) δ 0.40-0.44 (2H, m), 0.63-0.67 (2H, m), 1.60-1.64 (2H, q), 1.88-1.91 (2H, q), 2.54-2.57 (1H, m), 3.63-3.64 (4H, m), 3.68-3.70 (4H, m), 6.40-6.41 (1H, d), 6.68 (1H, s), 7.37-7.40 (2H, d), 7.57-7.61 (2H, t), 7.71-7.74 (1H, tt), 7.78-7.83 (4H, t), 8.50 (1H, s)

### EXAMPLE 36dd

¹H NMR (400.132 MHz, DMSO-d₆) δ 1.05-1.08 (3H, t), 1.60-1.64 (2H, q), 1.88-1.91 (2H, q), 3.09-3.16 (2H, m), 3.63-3.64 (4H, m), 3.68-3.70 (4H, m), 6.12-6.15 (1H, t), 6.68 (1H, s), 7.36-7.38 (2H, d), 7.57-7.61 (2H, t), 7.71-7.74 (1H, tt), 7.78-7.82 (4H, m), 8.62 (1H, s).

mTOR Kinase Assay (Echo): 0.00291 μM

### EXAMPLE 36de

¹H NMR (400.132 MHz, DMSO-d₆) δ 1.60-1.64 (2H, q), 1.88-1.91 (2H, q), 3.15-3.19 (2H, m), 3.44-3.48 (2H, q), 3.63-3.64 (4H, m), 3.68-3.70 (4H, m), 4.71-4.74 (1H, t), 6.21-6.24 (1H, t), 6.68 (1H, s), 7.36-7.38 (2H, d), 7.57-7.61 (2H, t), 7.70-7.74 (1H, tt), 7.78-7.83 (4H, m), 8.76 (1H, s).

mTOR Kinase Assay (Echo): 0.00389 μM

### EXAMPLE 36df

¹H NMR (400.132 MHz, DMSO-d₆) δ 1.60-1.64 (2H, q), 1.88-1.91 (2H, q), 2.65-2.66 (3H, d), 3.63-3.64 (4H, m), 3.68-3.69 (4H, m), 6.02-6.06 (1H, m), 6.68 (1H, s), 7.37-7.39 (2H, d), 7.57-7.61 (2H, t), 7.70-7.74 (1H, tt), 7.78-7.82 (4H, m), 8.70 (1H, s).

mTOR Kinase Assay (Echo): 0.00712 μM

### EXAMPLE 36dg

¹H NMR (400.132 MHz, DMSO-d₆) δ 1.56-1.64 (4H, m), 1.88-1.91 (2H, q), 3.14-3.19 (2H, q), 3.45-3.49 (2H, q), 3.63-3.64 (4H, m), 3.68-3.70 (4H, m), 4.46-4.49 (1H, t), 6.16-6.19 (1H, t), 6.68 (1H, s), 7.36-7.39 (2H, d), 7.57-7.61 (2H, t), 7.70-7.74 (1H, tt), 7.78-7.82 (4H, m), 8.67 (1H, s).

mTOR Kinase Assay (Echo): 0.0164 μM

### EXAMPLE 36dh

¹H NMR (400.132 MHz, DMSO-d₆) δ 1.60-1.64 (2H, q), 1.88-1.91 (2H, q), 2.68-2.72 (2H, t), 3.34-3.39 (2H, q), 3.63-3.64 (4H, m), 3.68-3.69 (4H, m), 6.49-6.52 (1H, m), 6.68 (1H, s), 7.38-7.41 (2H, d), 7.57-7.61 (2H, t), 7.70-7.74 (1H, tt), 7.78-7.84 (4H, m), 8.88 (1H, s).

mTOR Kinase Assay (Echo): 0.0034 μM

### EXAMPLE 36di

¹H NMR (400.132 MHz, DMSO-d₆) δ 1.61-1.65 (2H, q), 1.89-1.92 (2H, q), 3.63-3.66 (4H, m), 3.69-3.71 (4H, m), 6.73 (1H, m), 6.95 (1H, s), 7.08 (1H, s), 7.21 (1H, s), 7.49-7.52 (2H, d), 7.59-7.63 (2H, t), 7.70-7.74 (1H, tt), 7.79-7.91 (2H, d), 7.92-7.94 (2H, d), 8.38 (1H, s), 9.40 (1H, s), 11.39 (1H, s). (ammonium salt)

### EXAMPLE 36dj

¹H NMR (400.132 MHz, DMSO-d₆) δ 1.61-1.64 (2H, q), 1.89-1.92 (2H, q), 3.63-3.65 (4H, m), 3.69-3.70 (4H, m), 3.79 (3H, s), 6.69 (1H, s), 7.38-7.39 (1H, d), 7.41-7.44 (2H, d), 7.58-7.62 (2H, t), 7.71-7.75 (1H, tt), 7.77 (1H, s), 7.79-7.81 (2H, dd), 7.83-7.86 (2H, d), 8.36 (1H, s), 8.80 (1H, s)

### EXAMPLE 36dk

¹H NMR (400.132 MHz, DMSO-d₆) δ 0.40-0.44 (2H, m), 0.63-0.67 (2H, m), 1.21 (3H, d), 1.58-1.64 (2H, m), 1.86-1.88 (2H, m), 2.39 (3H, s), 2.54-2.58 (1H, m), 2.75-2.78 (4H, m), 3.11-3.18 (1H, m), 3.46-3.51 (1H, m), 3.62-3.66 (1H, m), 3.76 (1H, d), 3.95-3.98 (1H, m), 4.13-4.16 (1H, m), 4.48-4.54 (1H, m), 6.40 (1H, s), 6.66 (1H, s), 7.43 (2H, d), 7.64 (1H, s), 7.89 (2H, d), 8.51 (1H, s).

mTOR Kinase Assay (Echo): 0.00905 μM

### EXAMPLE 36dl

¹H NMR (400.132 MHz, DMSO-d₆) δ 1.21 (3H, d), 1.57-1.63 (2H, m), 1.84-1.89 (2H, m), 2.39 (3H, s), 2.74-2.79 (4H, m), 3.12-3.20 (3H, m), 3.44-3.51 (3H, m), 3.64 (1H, d), 3.76 (1H, d), 3.97 (1H, d), 4.12-4.16 (1H, m), 4.48-4.53 (1H, m), 4.72 (1H, t), 6.24 (1H, t), 6.66 (1H, s), 7.41 (2H, d), 7.64 (1H, s), 7.89 (2H, d), 8.78 (1H, s).

mTOR Kinase Assay (Echo): 0.00399 μM

### EXAMPLE 36dm

¹H NMR (400.132 MHz, DMSO-d₆) δ 1.21 (3H, d), 1.57-1.64 (2H, m), 1.84-1.90 (2H, m), 2.39 (3H, s), 2.70 (2H, t), 2.74-2.79 (4H, m), 3.12-3.18 (1H, m), 3.35-3.39 (2H, m), 3.46-3.51 (1H, m), 3.64 (1H, d), 3.76 (1H, d), 3.97 (1H, d), 4.15 (1H, d), 4.48-4.54 (1H, m), 6.51 (1H, t), 6.67 (1H, s), 7.44 (2H, d), 7.64 (1H, s), 7.90 (2H, d), 8.90 (1H, s).

mTOR Kinase Assay (Echo): 0.0298 μM

### EXAMPLE 36dn

¹H NMR (400.132 MHz, DMSO-d₆) δ 1.21 (3H, d), 1.57-1.61 (4H, m), 1.85-1.90 (2H, m), 2.39 (3H, s), 2.74-2.79 (4H, m), 3.12-3.19 (3H, m), 3.45-3.51 (3H, m), 3.64 (1H, d), 3.76 (1H, d), 3.97 (1H, d), 4.14 (1H, d), 4.47-4.54 (1H, m), 4.47 (1H, t), 6.18 (1H, t), 6.66 (1H, s), 7.41 (2H, d), 7.64 (1H, s), 7.89 (2H, d), 8.68 (1H, s).

mTOR Kinase Assay (Echo): 0.0138 μM

### EXAMPLE 36do

¹H NMR (400.132 MHz, DMSO-d₆) δ 1.17-1.19(3H, d), 1.61-1.68(2H, m), 1.78-1.86(1H, m), 1.88-1.98(3H, m), 3.10-3.18(1H, td), 3.31-3.33(1H, d), 3.43-3.49(4H, m), 3.59-3.63(1H, dd), 3.73-3.76(1H, d), 3.94-3.98(1H, dd), 4.10-4.13(1H, d), 4.31(1H, bs), 4.38(1H, bs), 4.94-4.95(1H, d), 6.63(1H, s), 7.52-7.55(2H, d), 7.58-7.61(2H, t), 7.70-7.74(1H, tt), 7.79-7.84(4H, m), 8.28(1H, s).

### EXAMPLE 36dp

¹H NMR (400.132 MHz, DMSO-d₆) δ 1.17-1.18(3H, d), 1.60-1.68(2H, m), 1.87-1.93(2H, m), 3.10-3.17(1H, td), 3.43-3.49(1H, td), 3.53-3.54(2H, m), 3.59-3.63(1H, dd), 3.73-3.76(1H, d), 3.94-3.97(1H, dd), 4.09-4.12(1H, d), 4.38(1H, bs), 6.47(1H, t), 6.63(1H, s), 7.39-7.41(2H, d), 7.57-7.61(2H, t), 7.70-7.74(1H, tt), 7.78-7.83(4H, m), 9.27(1H, s). (H from OH group barried under water peak).

### EXAMPLE 36dq

¹H NMR (400.132 MHz, DMSO-d₆) δ 1.16-1.18(3H, d), 1.61-1.68(2H, m), 1.87-1.92(2H, m), 2.88(3H, s), 2.97(3H, s), 3.10-3.18(1H, td), 3.42-3.49(1H, td), 3.59-3.63(1H, dd), 3.73-3.76(1H, d), 3.94-3.98(3H, m), 4.09-4.12(1H, d), 4.39(1H, bs), 6.37-6.40(1H, t), 6.63(1H, s), 7.38-7.40(2H, d), 7.57-7.61(2H, t), 7.69-7.74(1H, tt), 7.78-7.80(2H, dd), 7.84-7.86(2H, d), 9.12(1H, s).

### EXAMPLE 36dr

¹H NMR (400.132 MHz, DMSO-d₆) δ 0.68-0.72(2H, m), 0.88-0.93(2H, m), 1.17-1.19(3H, d), 1.61-1.68(2H, m), 1.89-1.92(2H, m), 2.69-2.75(1H, m), 2.87(3H, s), 3.11-3.18(1H, td), 3.43-3.50(1H, td), 3.59-3.63(1H, dd), 3.74-3.76(1H, d), 3.94-3.98(1H, dd), 4.09-4.14(1H, d), 4.39(1H, bs), 6.64(1H, s), 7.52-7.54(2H, d), 7.58-7.62(2H, t), 7.70-7.74(1H, tt), 7.79-7.85(4H, m), 8.34(1H, s).

### EXAMPLE 36ds

¹H NMR (400.132 MHz, DMSO-d₆) δ 1.16-1.18(3H, d), 1.60-1.68(2H, m), 1.87-1.93(2H, m), 3.10-3.18(1H, td), 3.36-3.40(1H, q), 3.42-3.49(2H, m), 3.59-3.63(1H, dd), 3.73-3.76(1H, d), 3.94-3.98(1H, dd), 4.09-4.12(1H, d), 4.39(1H, bs), 4.40-4.43(1H, t), 4.52-4.55(1H, t), 6.40-6.43(1H, t), 6.63(1H, s), 7.38-7.40(2H, d), 7.57-7.61(2H, t), 7.70-7.74(1H, tt), 7.78-7.80(2H, dd), 7.84-7.86(2H, d), 8.77(1H, s).

### EXAMPLE 36dt

¹H NMR (400.132 MHz, DMSO-d₆) δ 1.19-1.21(3H, d), 1.85-1.93(1H, m), 2.04-2.14(1H, m), 2.68-2.72(2H, t), 2.76-2.83(2H, m), 3.02-3.10(2H, m), 3.13-3.19(1H, td), 3.34-3.39(2H, q), 3.45-3.52(1H, td), 3.62-3.65(1H, dd), 3.74-3.77(1H, d), 3.94-3.98(1H, dd), 4.07-4.10(1H, d), 4.45(1H, bs), 6.47(1H, s), 6.49-6.52(1H, t), 7.38-7.40(2H, d), 7.44-7.51(4H, m), 7.58-7.62(1H, tt), 7.80-7.82(2H, d), 8.86(1H, s).

### EXAMPLE 36du

¹H NMR (400.132 MHz, DMSO-d₆) δ 1.19-1.21(3H, d), 1.82-1.93(1H, m), 2.05-2.13(1H, m), 2.76-2.83(2H, m), 3.02-3.19(4H, m), 3.45-3.51(1H, td), 3.62-3.65(1H, dd), 3.74-3.77(1H, d), 3.94-3.98(1H, dd), 4.06-4.10(1H, d), 4.31-4.33(2H, d), 4.45-4.46(1H, bs), 6.47(1H, s), 6.59-6.62(1H, t), 6.94(2H, bs), 7.38-7.40(2H, d), 7.44-7.51(4H, m), 7.58-7.62(1H, tt), 7.80-7.82(2H, d), 8.87(1H, s).

### EXAMPLE 36dv

¹H NMR (399.902 MHz, DMSO-d₆) δ 1.17 (3H, d), 1.64 (2H, m), 1.90 (2H, m), 3.15 (1H, m), 3.47 (1H, m), 3.61 (1H, m), 3.76 (4H, m), 3.95 (1H, m), 4.14 (1H, m), 4.39 (1H, m), 6.26 (1H, m), 6.65 (1H, s), 7.44 (2H, m), 7.59 (3H, m), 7.74 (1H, m), 7.80 (2H, m), 7.88 (2H, m), 8.94 (1H, s), 9.12 (1H, s)

### EXAMPLE 36dw

¹H NMR (399.902 MHz, DMSO-d₆) δ 1.18 (3H, d), 1.64 (2H, m), 1.91 (2H, m), 3.15 (1H, m), 3.34 (3H, s), 3.46 (1H, m), 3.61 (1H, m), 3.75 (1H, m), 3.96 (1H, m), 4.12 (1H, m), 4.40 (1H, m), 6.58 (1H, m), 6.66 (1H, m), 7.45 (2H, m), 7.60 (2H, m), 7.77 (3H, m), 7.90 (2H, m), 9.03 (1H, s), 9.49 (1H, s)

### EXAMPLE 36dx

¹H NMR (399.902 MHz, DMSO-d₆) δ 1.17 (3H, d), 1.64 (2H, m), 1.90 (2H, m), 3.14 (1H, m), 3.52 (4H, m), 3.75 (1H, m), 3.96 (1H, m), 4.11 (1H, m), 4.38 (1H, m), 6.08 (1H, m), 6.53 (1H, m), 6.63 (1H, s), 7.40 (2H, m), 7.60 (2H, m), 7.72 (1H, m), 7.82 (4H, m), 8.92 (1H, s)

### EXAMPLE 36dy

Spectrum not recorded.

### EXAMPLE 36dz

¹H NMR (399.902 MHz, DMSO-d₆) δ 1.18 (3H, d), 1.64 (2H, m), 1.90 (2H, m), 3.15 (1H, m), 3.47 (1H, m), 3.61 (1H, m), 3.75 (1H, m), 3.96 (1H, m), 4.13 (1H, m), 4.40 (1H, m), 6.66 (1H, s), 6.88 (1H, m), 7.46 (2H, m), 7.61 (2H, m), 7.77 (3H, m), 7.90 (2H, m), 8.77 (1H, m), 9.05 (1H, s), 9.64 (1H, s)

### EXAMPLE 36ea

¹H NMR (399.902 MHz, DMSO-d₆) δ 1.17 (3H, d), 1.64 (2H, m), 1.91 (2H, m), 3.14 (1H, m), 3.46 (1H, m), 3.61 (1H, m), 3.75 (1H, m), 3.94 (3H, m), 4.12 (1H, m), 4.39 (1H, m), 6.64 (1H, s), 6.79 (1H, m), 7.40 (2H, m), 7.59 (2H, m), 7.79 (5H, m), 8.99 (1H, s)

### EXAMPLE 36eb

¹H NMR (399.902 MHz, DMSO-d₆) δ 1.18 (3H, d), 1.65 (2H, m), 1.91 (2H, m), 3.15 (1H, m), 3.46 (1H, m), 3.61 (4H, m), 3.75 (1H, m), 3.97 (1H, m), 4.12 (1H, m), 4.40 (1H, m), 6.64 (1H, s), 6.97 (1H, s), 7.41 (3H, m), 7.61 (2H, m), 7.80 (5H, m), 8.70 (1H, s), 9.07 (1H, s)

### EXAMPLE 36ec

¹H NMR (399.902 MHz, DMSO-d₆) δ 1.08 (3H, d), 1.17 (3H, d), 1.64 (2H, m), 1.90 (2H, m), 3.13 (1H, m), 3.40 (3H, m), 3.67 (3H, m), 3.96 (1H, m), 4.10 (1H, m), 4.37 (1H, m), 4.83 (1H, t), 6.10 (1H, m), 6.63 (1H, s), 7.36 (2H, m), 7.60 (2H, m), 7.78 (5H, m), 8.70 (1H, s)

### EXAMPLE 36ed

¹H NMR (399.902 MHz, DMSO-d₆) δ 1.08 (3H, d), 1.17 (3H, d), 1.64 (2H, m), 1.91 (2H, m), 3.14 (1H, m), 3.42 (3H, m), 3.67 (3H, m), 3.96 (1H, m), 4.11 (1H, m), 4.39 (1H, m), 4.83 (1H, t), 6.10 (1H, m), 6.62 (1H, s), 7.36 (2H, m), 7.59 (2H, m), 7.77 (5H, m), 8.71 (1H, s)

### EXAMPLE 36ee

¹H NMR (399.902 MHz, DMSO-d₆) δ 1.17 (3H, d), 1.63 (2H, m), 1.90 (2H, m), 3.13 (1H, m), 3.45 (3H, m), 3.67 (4H, m), 3.95 (1H, m), 4.12 (1H, m), 4.39 (1H, m), 6.47 (1H, m), 6.63 (1H, s), 7.39 (2H, m), 7.60 (2H, m), 7.79 (5H, m), 8.91 (1H, s)

### EXAMPLE 36ef

¹H NMR (399.902 MHz, DMSO-d₆) δ 1.23 (d, 3H), 1.33 (t, 3H), 1.52-1.58 (m, 2H), 1.60-1.66 (m, 2H), 3.20 (td, 1H), 3.37-3.53 (m, 5H), 3.63 (dd, 1H), 3.77 (d, 1H), 3.98 (dd, 1H), 4.14-4.28 (m, 1H), 4.42 (t, 1H), 4.50-4.62 (m, 1H), 4.54 (t, 1H), 6.47 (t, 1H), 6.79 (s, 1H), 7.51 (d, 2H), 8.20 (d, 2H), 8.85 (s, 1H)

### EXAMPLE 36eg

¹H NMR (399.902 MHz, DMSO-d₆) δ 1.23 (d, 3H), 1.33 (t, 3H), 1.53-1.58 (m, 2H), 1.60-1.66 (m, 2H), 3.20 (td, 1H), 3.44 (q, 2H), 3.48-3.65 (m, 4H), 3.77 (d, 1H), 3.98 (dd, 1H), 4.16-4.26 (m, 1H), 4.51-4.64 (m, 1H), 6.07 (tt, 1H), 6.56 (t, 1H), 6.79 (s, 1H), 7.52 (d, 2H), 8.21 (d, 2H), 8.97 (s, 1H)

The preparation of the phenyl carbamates required for Examples 36a-36eg are either described below or have been described previously.

The preparation of phenyl N-[4-[4-(1-cyclopropylsulfonylcyclobutyl)-6-[(3S)-3-methylmorpholin-4-yl]pyrimidin-2-yl]phenyl]carbamate is described below.

### Phenyl N-[4-[4-(1-cyclopropylsulfonylcyclobutyl)-6-[(3S)-3-methylmorpholin-4-yl]pyrimidin-2-yl]phenyl]carbamate

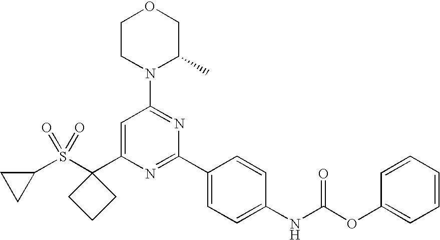

Sodium hydrogen carbonate (259 mg, 3.08 mmol) was added to 4-[4-(1-cyclopropylsulfonylcyclobutyl)-6-[(3S)-3-methylmorpholin-4-yl]pyrimidin-2-yl]aniline (880 mg, 2.05 mmol) in dioxane (20 mL) at 5° C. under an atmosphere of nitrogen. Phenyl chloroformate (0.387 mL, 3.08 mmol) was then added and the resulting mixture was stirred at RT for 2 hours. The reaction mixture was diluted with DCM (50 mL), the organic layer dried (Na₂SO₄), filtered and evaporated to afford crude product. The crude gum was triturated with a mixture of diethyl ether and isohexane to give the desired material as a cream solid which was collected by filtration and dried under vacuum (1.06 g).

NMR Spectrum: ¹H NMR (400.132 MHz, DMSO-d₆) δ 0.69-0.80 (2H, m), 0.81-0.90 (2H, m), 1.23 (3H, d), 1.86-1.98 (1H, m), 2.02-2.12 (1H, m), 2.80-3.04 (5H, m), 3.15-3.28 (1H, m), 3.46-3.59 (1H, m), 3.66 (1H, d), 3.77 (1H, d), 3.98 (1H, d), 4.23 (1H, d), 4.57 (1H, s), 6.74 (1H, s), 7.22-7.31 (3H, m), 7.41-7.49 (2H, m), 7.62 (2H, d), 8.33 (2H, d), 10.42 (1H, s)

LCMS Spectrum: m/z (ESI+)(M+H)+=549; HPLC tR=3.05 min.

### 4-[4-(1-Cyclopropylsulfonylcyclobutyl)-6-[(3s)-3-methylmorpholin-4-yl]pyrimidin-2-yl]aniline

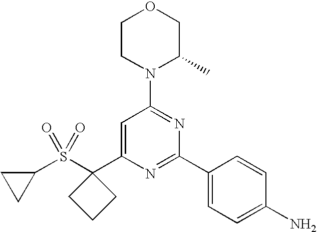

Bis(triphenylphosphine)palladium(II) chloride (0.164 g, 0.23 mmol) was added to 2-chloro-4-(1-cyclopropylsulfonylcyclobutyl)-6-[(3S)-3-methylmorpholin-4-yl]pyrimidine (1.3 g, 3.50 mmol), 4-(4,4,5,5-tetramethyl-1,3,2-dioxaborolan-2-yl)aniline (1.149 g, 5.24 mmol) and an aqueous solution of sodium carbonate (1 mL, 2.00 mmol) in a solvent mixture of DMF (5 mL), DME (12 mL), water (1 mL) and ethanol (1 mL) at RT . The resulting mixture was stirred at 90° C. for 5 hours under an atmosphere of nitrogen. The reaction mixture was diluted with ethyl acetate (100 mL), and washed with water (2×100 mL). The organic layer was dried (Na₂SO₄), filtered and evaporated to afford crude product. The crude product was purified by flash silica chromatography, elution gradient 10 to 70% ethyl acetate in isohexane, and the crude product further purified by ion exchange chromatography using an SCX column, eluting with 7N ammonia in methanol, to give the desired material as a beige solid (0.88 g).

NMR Spectrum: ¹H NMR (400.132 MHz, DMSO-d₆) δ 0.70-0.89 (4H, m), 1.21 (3H, d), 1.84-1.93 (1H, m), 2.02-2.10 (1H, m), 2.76-2.98 (5H, m), 3.10-3.24 (1H, m), 3.45-3.55 (1H, m), 3.64 (1H, d), 3.76 (1H, d), 3.97 (1H, d), 4.18 (1H, d), 4.50 (1H, s), 5.52 (2H, d), 6.60 (2H, d), 8.07 (2H, d)

LCMS Spectrum: m/z (ESI+)(M+H)+=429; HPLC tR=2.41 min.

### 2-Chloro-4-(1-cyclopropylsulfonylcyclobutyl)-6-[(3S)-3-methylmoipholin-4-yl]pyrimidine

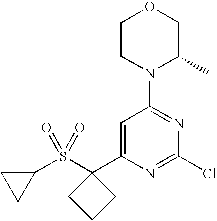

1,3-Dibromopropane (2.95 mL, 28.93 mmol) was added to 2-chloro-4-(cyclopropylsulfonylmethyl)-6-[(3S)-3-methylmorpholin-4-yl]pyrimidine (3.2 g, 9.64 mmol), tetrabutylammonium bromide (0.311 g, 0.96 mmol) and an aqueous solution of sodium hydroxide (2.89 mL, 28.93 mmol) in toluene (24.11 mL). The reaction was stirred at RT for 1 hour then water added and the layers separated. The organic layer was dried (MgSO₄), filtered and evaporated to afford crude product. The crude product was purified by flash silica chromatography, elution gradient 0 to 60% ethyl acetate in isohexane, to give the desired material as a colourless gum (1.3 g).

NMR Spectrum: ¹H NMR (400.132 MHz, DMSO-d₆) δ 0.69-0.78 (2H, m), 0.88-0.93 (2H, m), 1.20 (3H, d), 1.83-1.95 (1H, m), 2.02-2.12 (1H, m), 2.50-2.60 (1H, m), 2.67-2.80 (2H, m), 2.83-2.96 (2H, m), 3.13-3.25 (1H, m), 3.40-3.49 (1H, m), 3.61 (1H, d), 3.72 (1H, d), 3.93 (1H, d), 4.06 (1H, s), 4.40 (1H, s), 6.82 (1H, s)

LCMS Spectrum: m/z (ESI+)(M+H)+=372; HPLC tR=2.04 min.

The preparation of ²-chloro-4-(cyclopropylsulfonylmethyl)-6-[(3S)-3-methylmorpholin-4-yl]pyrimidine was described earlier.

The preparation of phenyl N-[4-[4-(1-cyclopropylsulfonylcyclopentyl)-6-[(3s)-3-methylmorpholin-4-yl]pyrimidin-2-yl]phenyl]carbamate is described below.

### Phenyl N-[4-[4-(1-cyclopropylsulfonylcyclopentyl)-6-[(3S)-3-methylmorpholin-4-yl]pyrimidin-2-yl]phenyl]carbamate

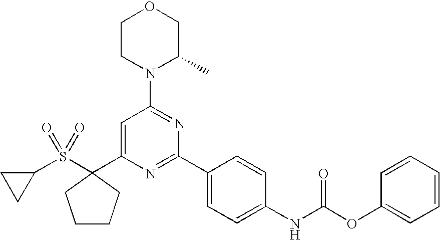

Sodium hydrogen carbonate (228 mg, 2.71 mmol) was added to 4-[4-(1-cyclopropylsulfonylcyclopentyl)-6-[(3S)-3-methylmorpholin-4-yl]pyrimidin-2-yl]aniline (800 mg, 1.81 mmol) in dioxane (20 mL) at 5° C. under an atmosphere of nitrogen. Phenyl chloroformate (0.341 mL, 2.71 mmol) was then added. The resulting mixture was stirred at RT for 2 hours then diluted with ethyl acetate (100 mL). The organic layer was dried (Na₂SO₄), filtered and evaporated to afford crude product which was triturated with a mixture of diethyl ether and isohexane to give the desired material as a cream solid which was collected by filtration and dried under vacuum (700 mg).

NMR Spectrum: ¹H NMR (400.132 MHz, DMSO-d₆) δ 0.57-0.78 (2H, m), 0.79-0.90 (2H, m), 1.23 (3H, d), 1.53-1.62 (2H, m), 1.77-1.87 (2H, m), 2.41-2.50 (2H, m), 2.55-2.62 (1H, m), 2.76-2.91 (2H, m), 3.16-3.26 (1H, m), 3.45-3.56 (1H, m), 3.66 (1H, d), 3.77 (1H, d), 3.98 (1H, d), 4.24 (1H, d), 4.55 (1H, s), 6.84 (1H, s), 7.20-7.34 (3H, m), 7.41-7.51 (2H, m), 7.62 (2H, d), 8.34 (2H, d), 10.42 (1H, s)

LCMS Spectrum: m/z (ESI+)(M+H)+=563; HPLC tR=3.15 min.

### 4-[4-(1-Cyclopropylsulfonylcyclopentyl)-6-[(3S)-3-methylmorpholin-4-yl]pyrimidin-2-yl]aniline

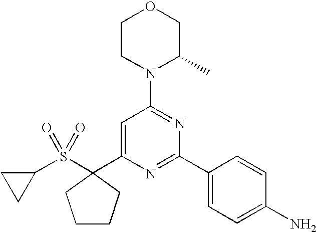

Bis(triphenylphosphine)palladium(II) chloride (0.122 g, 0.17 mmol) was added to 2-chloro-4-(1-cyclopropylsulfonylcyclopentyl)-6-[(3S)-3-methylmorpholin-4-yl]pyrimidine (1 g, 2.59 mmol), and an aqueous solution of sodium carbonate (1 mL, 2.00 mmol) in a solvent mixture of DMF (5 mL), DME (12 mL), water (1 mL) and ethanol (1 mL). The resulting mixture was stirred at 90° C. for 5 hours under an inert atmosphere. The reaction mixture was diluted with ethyl acetate (100 mL), and washed with water (2×100 mL). The organic layer was dried (Na₂SO₄), filtered and evaporated to afford crude product which was purified by flash silica chromatography, elution gradient 10 to 50% ethyl acetate in isohexane, followed by ion exchange chromatography using an SCX column, eluting with 7N ammonia in methanol, to give the desired material as a beige solid (0.80 g).

NMR Spectrum: ¹H NMR (400.132 MHz, DMSO-d₆) δ 0.67-0.79 (2H, m), 0.81-0.89 (2H, m), 1.22 (3H, d), 1.50-1.59 (2H, m), 1.75-1.85 (2H, m), 2.41-2.51 (2H, m), 2.72-2.90 (3H, m), 3.11-3.23 (1H, m), 3.45-3.56 (1H, m), 3.58-3.66 (1H, m), 3.76 (1H, d), 3.97 (1H, d), 4.18 (1H, d), 4.51 (1H, s), 5.52 (1H, d), 6.61 (2H, d), 6.71 (1H, s), 8.07 (2H, d)

LCMS Spectrum: m/z (ESI+)(M+H)+=443; HPLC tR=2.51 min.

### 2-Chloro-4-(1-cyclopropylsulfonylcyclopentyl)-6-[(3S)-3-methylmorpholin-4-yl pyrimidine

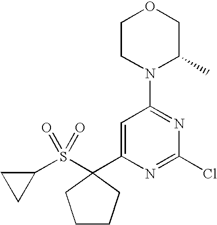

1,4-Dibromobutane (0.322 mL, 2.71 mmol) was added to 2-chloro-4-(cyclopropylsulfonylmethyl)-6-[(3S)-3-methylmorpholin-4-yl]pyrimidine (900 mg, 2.71 mmol), tetrabutylammonium bromide (87 mg, 0.27 mmol) and an aqueous solution of sodium hydroxide (0.814 mL, 8.14 mmol) in toluene (20 mL). The reaction was stirred at RT for 1 hour then water added and the organic layer separated, dried (MgSO₄), filtered and evaporated to afford crude product. The crude product was purified by flash silica chromatography, elution gradient 0 to 60% ethyl acetate in isohexane, to give the desired material as a yellow gum (1043 mg).

NMR Spectrum: ¹H NMR (400.132 MHz, DMSO-d₆) δ 0.66-0.75 (2H, m), 0.88-0.94 (2H, m), 1.20 (3H, d), 1.50-1.57 (2H, m), 1.74-1.83 (2H, m), 2.36-2.46 (2H, m), 2.54-2.69 (3H, m), 3.13-3.25 (1H, m), 3.40-3.50 (1H, m), 3.59 (1H, d), 3.72 (1H, d), 3.93 (1H, d), 4.04 (1H, d), 4.41 (1H, s), 6.92 (1H, s)

LCMS Spectrum: m/z (ESI+)(M+H)+=386; HPLC tR=2.47 min.

The preparation of 2-chloro-4-(cyclopropylsulfonylmethyl)-6-[(3S)-3-methylmorpholin-4-yl]pyrimidine was described earlier.

The preparation of phenyl N-[4-[4-[(3S)-3-methylmorpholin-4-yl]-6-(1-pyridin-2-ylsulfonylcyclopentyl)pyrimidin-2-yl]phenyl]carbamate is described below:

### Phenyl N-[4-[4-[(3S)-3-methylmorpholin-4-yl]-6-(1-pyridin-2-ylsulfonylcyclopentyl)pyrimidin-2-yl]phenyl]carbamate

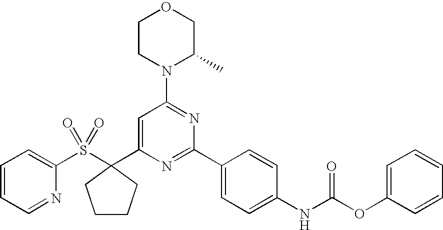

Sodium bicarbonate (139 mg, 1.66 mmol) and phenyl chloroformate (0.14 mL, 1.11 mmol) were added to a solution of 4-[4-[(3S)-3-methylmorpholin-4-yl]-6-(1-pyridin-2-ylsulfonylcyclopentyl)pyrimidin-2-yl]aniline (530 mg, 1.11 mmol), in 1,4-dioxane (5.6 mL) and the reaction stirred at RT for 2 hours. The reaction mixture was evaporated to dryness and redissolved in DCM (10 mL), washed with water (10 mL), the organic layer dried (MgSO₄), filtered and evaporated. The crude product was triturated with diethyl ether to give the desired material as a cream solid (620 mg).

NMR Spectrum: ¹H NMR (400.132 MHz, DMSO-d₆) δ 1.20 (3H, d), 1.51-1.62 (2H, m), 1.80-1.90 (2H, m), 2.68-2.82 (4H, m), 3.10-3.17 (1H, m), 3.44-3.50 (1H, m), 3.61-3.64 (1H, m), 3.75 (1H, d), 3.94-3.97 (1H, m), 4.12 (1H, d), 4.49 (1H, s), 6.64 (1H, s), 7.24-7.30 (3H, m), 7.45 (2H, t), 7.50 (2H, d), 7.55-7.59 (2H, m), 7.84-7.86 (3H, m), 8.74 (1H, d), 10.37 (1H, s)

LCMS Spectrum: m/z (ESI+) (M+H)+=600; HPLC tR=3.05 min.

### 4-[4-[(3S)-3-Methylmorpholin-4-yl]-6-(1-pyridin-2-ylsulfonylcyclopentyl)pyrimidin-2-yl]aniline

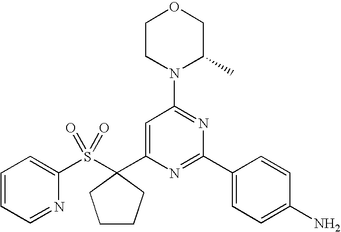

4-(4,4,5,5-Tetramethyl-1,3,2-dioxaborolan-2-yl)aniline (418 mg, 1.91 mmol), an aqueous solution of sodium carbonate (2.2 mL, 4.40 mmol), and dichlorobis(triphenylphosphine)palladium(II) (51.4 mg, 0.07 mmol) were added to a solution of 2-chloro-4-[(3S)-3-methylmorpholin-4-yl]-6-(1-pyridin-2-ylsulfonylcyclopentyl)pyrimidine (620 mg, 1.47 mmol) in a solvent mixture of DMF (0.24 mL), DME (9.33 mL), water (4.0 mL) and ethanol (2.67 mL). The resultant mixture was heated at 90° C. for 4 hours. The reaction mixture was cooled to RT, diluted with ethyl acetate (10 mL) and washed with water (10 mL). The organic layer was dried (MgSO₄), filtered and evaporated. The crude product was purified by flash silica chromatography, elution gradient 0 to 10% methanol in DCM, to give the desired material as a cream solid (530 mg).

NMR Spectrum: ¹H NMR (400.132 MHz, DMSO-d₆) δ 1.17 (3H, d), 1.51-1.58 (2H, m), 1.80-1.88 (2H, m), 2.68-2.79 (4H, m), 3.09 (1H, dt), 3.46 (1H, dt), 3.61 (1H, dd), 3.74 (1H, d), 3.94 (1H, dd), 4.01-4.06 (1H, m), 4.43 (1H, d), 6.46-6.49 (3H, m), 7.54 (1H, d), 7.57-7.60 (1H, m), 7.60 (2H, d), 7.85 (1H, dt), 8.74 (1H, d)

LCMS Spectrum: m/z (ESI+) (M+H)+=480; HPLC tR=2.37 min.

### 2-Chloro-4-[(3S)-3-methylmorpholin-4-yl]-6-(1-pyridin-2-ylsulfonylcyclopentyl)pyrimidine

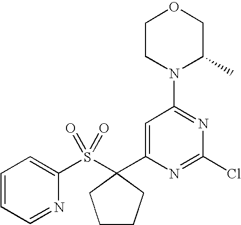

1,4-dibromobutane (0.77 mL, 6.51 mmol) was added to 2-chloro-4-[(3S)-3-methylmorpholin-4-yl]-6-(pyridin-2-ylsulfonylmethyl)pyrimidine (600 mg, 1.63 mmol) in toluene (4 mL) followed by tetrabutylammonium bromide (52.4 mg, 0.16 mmol) and an aqueous solution of sodium hydroxide (0.976 mL, 9.76 mmol). The reaction was stirred at 60° C. overnight then the toluene removed under reduced pressure and the reaction redissolved in DCM and washed with water. The organic layer was dried (MgSO₄), filtered and evaporated to afford crude product. The crude product was purified by flash silica chromatography, elution gradient 0 to 50% ethyl acetate in DCM, to give the desired material as a white solid (620 mg).

NMR Spectrum: ¹H NMR (400.132 MHz, DMSO-d₆) δ 1.16 (3H, d), 1.50-1.58 (2H, m), 1.75-1.85 (2H, m), 2.53-2.57 (2H, m), 2.63-2.71 (3H, m), 3.11 (1H, dt), 3.41 (1H, dt), 3.56 (1H, dd), 3.70 (1H, d), 3.91 (1H, dd), 4.32 (1H, s), 6.67 (1H, s), 7.65 (1H, d), 7.70-7.73 (1H, m), 8.03 (1H, dt), 8.74 (1H, d)

LCMS Spectrum: m/z (ESI+)(M+H)+ 423, HPLC tR=2.26 min

The preparation of 2-chloro-4-[(3S)-3-methylmorpholin-4-yl]-6-(pyridin-2-ylsulfonylmethyl)pyrimidine was described earlier.

The preparation of phenyl N-[4-[4-[(3S)-3-methylmorpholin-4-yl]-6-(1-pyridin-2-ylsulfonylcyclobutyl)pyrimidin-2-yl]phenyl]carbamate is described below:

### Phenyl N-[4-[4-[(3S)-3-methylmorpholin-4-yl]-6-(1-pyridin-2-ylsulfonylcyclobutyl)pyrimidin-2-yl]phenyl]carbamate

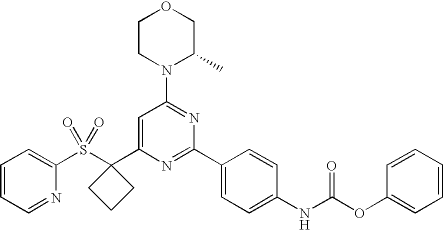

Sodium bicarbonate (154 mg, 1.84 mmol) and phenyl chloroformate (0.154 mL, 1.22 mmol) were added to a solution of 4-[4-[(3S)-3-methylmorpholin-4-yl]-6-(1-pyridin-2-ylsulfonylcyclobutyl)pyrimidin-2-yl]aniline (570 mg, 1.22 mmol), in 1,4-dioxane (6.0 mL) and the reaction stirred at RT for 2 hours. The reaction mixture was evaporated to dryness and redissolved in DCM (10 mL), washed with water (10 mL), the organic layer dried (MgSO₄), filtered and evaporated. The crude product was triturated with diethyl ether to give the desired material as a cream solid (600 mg).

NMR Spectrum: ¹H NMR (400.132 MHz, DMSO-d₆) δ 1.19 (3H, d), 1.86-1.98 (1H, m), 2.07-2.19 (1H, m), 2.80-2.87 (2H, m), 3.10-3.17 (1H, m), 3.23-3.30 (2H, m), 3.47 (1H, dt), 3.62 (1H, dd), 3.75 (1H, d), 3.96 (1H, dd), 4.10-4.18 (1H, m), 4.46 (1H, s), 6.52 (1H, s), 6.74-6.78 (1H, m), 7.15 (1H, t), 7.23-7.30 (3H, m), 7.45 (1H, t), 7.51 (1H, d), 7.60-7.62 (2H, m), 7.83-7.90 (3H, m), 8.74 (1H, d), 10.39 (1H, s)

LCMS Spectrum: m/z (ESI+) (M+H)+=586; HPLC tR=3.04 min.

### 4-[4-[(3S)-3-Methylmorpholin-4-yl]-6-(1-pyridin-2-ylsulfonylcyclobutyl)pyrimidin-2-yl]aniline

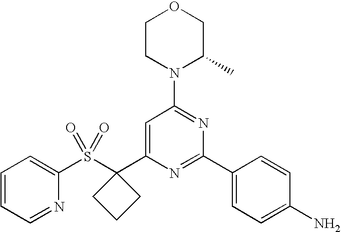

4-(4,4,5,5-Tetramethyl-1,3,2-dioxaborolan-2-yl)aniline (460 mg, 2.10 mmol), an aqueous solution of sodium carbonate (2.42 mL, 4.84 mmol) and

dichlorobis(triphenylphosphine)palladium(II) (56.6 mg, 0.08 mmol) were added to a solution of 2-chloro-4-[(3S)-3-methylmorpholin-4-yl]-6-(1-pyridin-2-ylsulfonylcyclobutyl)pyrimidine (660 mg, 1.61 mmol) in a solvent mixture of DMF (0.24 mL), DME (9.33 mL), water (4.0 mL) and ethanol (2.67 mL) and the suspension heated at 90° C. for 4 hours. The reaction mixture was cooled to RT, diluted with ethyl acetate (10 mL) and washed with water (10 mL). The organic layer was dried (MgSO₄), filtered and evaporated. The crude product was purified by flash silica chromatography, elution gradient 0 to 10% methanol in DCM, to give the desired material as a cream solid (570 mg).

LCMS Spectrum: m/z (ESI+) (M+H)+=466; HPLC tR=2.27 min.

### 2-Chloro-4-[(3S)-3-methylmorpholin-4-yl]-6-(1-pyridin-2-ylsulfonylcyclobutyl)pyrimidine

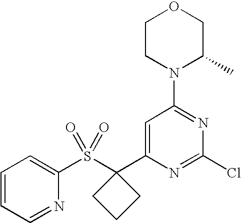

1,3-Dibromopropane (1.565 mLl, 15.35 mmol) was added to 2-chloro-4-[(3S)-3-methylmorpholin-4-yl]-6-(pyridin-2-ylsulfonylmethyl)pyrimidine (2.83 g, 7.67 mmol), in toluene (20 mL) followed by tetrabutylammonium bromide (0.247 g, 0.77 mmol) and an aqueous solution of sodium hydroxide (2.3 mL, 23.02 mmol). The reaction was stirred at 60° C. overnight. The toluene was removed under reduced pressure and the reaction redissolved in DCM and the organics washed with water then dried (MgSO₄), filtered and evaporated to afford crude product. The crude product was purified by flash silica chromatography, elution gradient 0 to 50% ethyl acetate in DCM, to give the desired material as a cream solid (0.66 g).

NMR Spectrum: ¹H NMR (400.132 MHz, DMSO-d₆) δ 1.14 (3H, d), 1.86-1.95 (1H, m), 2.05-2.13 (1H, m), 2.68-2.76 (2H, m), 3.07-3.18 (3H, m), 3.37-3.43 (1H, m), 3.55 (1H, dd), 3.69 (1H, d), 3.90 (2H, dd), 4.28 (1H, s), 6.52 (1H, s), 7.68-7.73 (2H, m), 8.03 (1H, dt), 8.74 (1H, d)

LCMS Spectrum: m/z (ESI+)(M+H)+ 409, HPLC tR=2.03 min

The preparation of 2-chloro-4-[(3S)-3-methylmorpholin-4-yl]-6-(pyridin-2-ylsulfonylmethyl)pyrimidine was described earlier.

The preparation of phenyl N-[4-[4-[1-(4-fluorophenyl)sulfonylcyclopropyl]-6-morpholin-4-ylpyrimidin-2-yl]phenyl]carbamate is described below:

### Phenyl N-[4-[4-[1-(4-fluorophenyl)sulfonylcyclopropyl]-6-morpholin-4-ylpyrimidin-2-yl]phenyl]carbamate

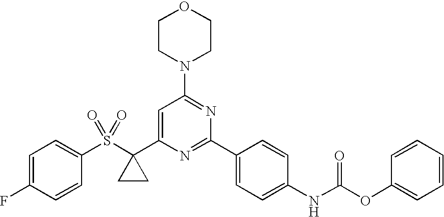

Sodium bicarbonate (0.776 g, 9.24 mmol) was added to 4-[4-[1-(4-fluorophenyl)sulfonylcyclopropyl]-6-morpholin-4-ylpyrimidin-2-yl]aniline (2.8 g, 6.16 mmol), in 1,4-dioxane (30.8 mL) at RT, followed by the dropwise addition of phenyl chloroformate (0.775 ml, 6.16 mmol) over 2 minutes and the reaction stirred at RT for 2 hours. The reaction mixture was evaporated to dryness and redissolved in DCM (20 mL), and washed with water (20 mL). The organic layer was dried (MgSO₄), filtered and evaporated to afford a solid which was triturated with diethyl ether to give the desired material as a cream solid (3.5 g).

NMR Spectrum: ¹H NMR (400.132 MHz, DMSO-d₆) δ 1.59-1.62 (2H, m), 1.88-1.91 (2H, m), 3.57 (4H, s), 3.69 (4H, s), 6.75 (1H, s), 7.24-7.30 (3H, m), 7.40-7.47 (4H, m), 7.53 (2H, d), 7.83-7.89 (4H, m), 10.40 (1H, s)

LCMS Spectrum: m/z (ESI+) (M+H)+=575; HPLC tR=3.03 min.

### 4-[4-[1-(4-Fluorophenyl)sulfonylcyclopropyl]-6-morpholin-4-ylpyrimidin-2-yl]aniline

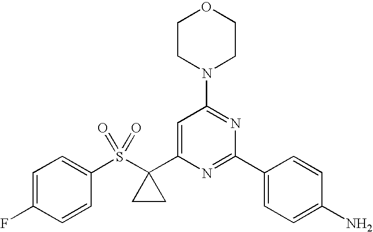

Bis(triphenylphosphine)palladium(II) chloride (0.285 g, 0.41 mmol) was added to 4-(4,4,5,5-tetramethyl-1,3,2-dioxaborolan-2-yl)aniline (2.312 g, 10.55 mmol) and 2-chloro-4-[1-(4-fluorophenyl)sulfonylcyclopropyl]-6-morpholin-4-ylpyrimidine (3.23 g, 8.12 mmol) and an aqueous solution of sodium carbonate (12.18 mL, 24.36 mmol) in a solvent mixture of DME (20 mL), ethanol (10 mL) and water (10 mL) at RT under an atmosphere of nitrogen. The resulting mixture was stirred at 95° C. for 4 hours. The reaction mixture was allowed to cool, diluted with ethyl acetate (20 mL), and washed with water (2×20mL). The organic layer was dried (MgSO₄), filtered and evaporated to afford crude product. The crude product was purified by flash silica chromatography, elution gradient 0 to 10% methanol in DCM, to give the desired material as a cream solid (2.8 g).

NMR Spectrum: ¹H NMR (400.132 MHz, DMSO-d₆) δ 1.55-1.58 (2H, m), 1.85-1.88 (2H, m), 3.62 (4H, d), 3.67-3.70 (4H, m), 6.49 (2H, d), 6.61 (1H, s), 7.41 (2H, t), 7.62 (2H, d), 7.82-7.85 (2H, m)

LCMS Spectrum: m/z (ESI+) (M+H)+=455; HPLC tR=2.42 min.

### 2-Chloro-4-[1-(4-fluorophenyl)sulfonylcyclopropyl]-6-morpholin-4-ylpyrimidine

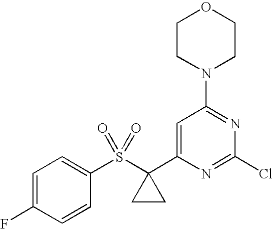

1,2-Dibromoethane (0.695 mL, 8.07 mmol) was added to 2-chloro-4-[(4-fluorophenyl)sulfonylmethyl]-6-morpholin-4-ylpyrimidine (3 g, 8.07 mmol), tetrabutylammonium bromide (0.260 g, 0.81 mmol) and an aqueous solution of sodium hydroxide (2.42 mL, 24.21 mmol) in toluene (20.17 mL). The reaction was stirred at RT for 4 hours, the toluene removed under reduced pressure and the residue redissolved in DCM. The organics were washed with water, dried (MgSO₄), filtered and evaporated to afford crude product. The crude product was triturated with diethyl ether to give the desired material as a cream solid (3.23 g).

NMR Spectrum: ¹H NMR (400.132 MHz, DMSO-d₆) δ 1.52-1.55 (2H, m), 1.81-1.84 (2H, m), 3.56 (4H, s), 3.63-3.65 (4H, m), 6.77 (1H, s), 7.80-7.84 (2H, m), 7.82 (2H, t)

LCMS Spectrum: m/z (ESI+)(M+H)+ 398, HPLC tR=2.26 min

### 2-Chloro-4-[(4-fluorophenyl)sulfonylmethyl]-6-morpholin-4-ylpyrimidine

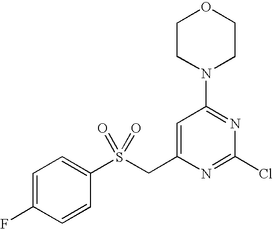

4-Fluorobenzenesulfinic acid sodium salt (3.30 g, 18.11 mmol) was added to 2-chloro-4-(iodomethyl)-6-morpholin-4-ylpyrimidine (5.00 g, 14.72 mmol) in acetonitrile (150 mL) at RT under an atmosphere of nitrogen. The resulting solution was stirred at 80° C. for 20 hours. The solvent was removed and the residue redissolved in DCM. The organics were washed twice with water, dried (MgSO₄) and filtered. The crude product was purified by flash silica chromatography, elution gradient 0 to 30% ethyl acetate in DCM, to give the desired material as a white solid (3.98 g).

NMR Spectrum: ¹H NMR (400.132 MHz, DMSO-d₆) δ 3.55-3.67 (8H, m), 4.65 (2H, s), 6.78 (1H, s), 7.47-7.52 (2H, m), 7.84-7.87 (2H, m).

LCMS Spectrum: m/z (ESI+)(M+H)+=372; HPLC tR=1.99 min.

The preparation of 2-chloro-4-(iodomethyl)-6-morpholin-4-ylpyrimidine was described earlier.

The preparation of phenyl N-[4-[4-morpholin-4-yl-6-(1-pyridin-4-ylsulfonylcyclopropyl)pyrimidin-2-yl]phenyl]carbamate is described below:

### Phenyl N-[4-[4-morpholin-4-yl-6-(1-pyridin-4-ylsulfonylcyclopropyl)pyrimidin-2-yl]phenyl]carbamate

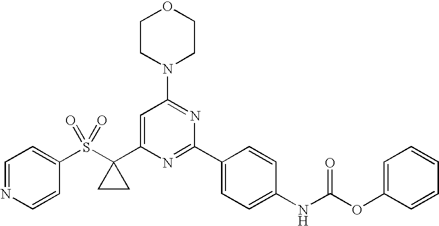

Sodium bicarbonate (0.677 g, 8.06 mmol) was added to 4-[4-morpholin-4-yl-6-(1-pyridin-4-ylsulfonylcyclopropyl)pyrimidin-2-yl]aniline (2.35 g, 5.37 mmol), in 1,4-dioxane (26.9 mL) at RT, followed by the dropwise addition of phenyl chloroformate (0.676 mL, 5.37 mmol) over 2 minutes and the reaction stirred at RT for 2 hours. The reaction mixture was evaporated to dryness and redissolved in DCM (20 mL), and washed with water (20 mL). The organic layer was dried (MgSO₄), filtered and evaporated to afford a solid which was triturated with diethyl ether to give the desired material as a cream solid (3.0 g).

NMR Spectrum: ¹H NMR (400.132 MHz, DMSO-d₆) δ 1.67-1.70 (2H, m), 1.94-1.99 (2H, m), 3.57 (4H, s), 3.69 (4H, s), 6.74-6.77 (2H, m), 7.24-7.26 (2H, m), 7.43-7.50 (3H, m), 7.73 (2H, d), 7.78 (2H, d), 8.86 (2H, d), 9.29 (1H, s), 10.39 (1H, s)

LCMS Spectrum: m/z (ESI+) (M+H)+=558; HPLC tR=2.71 min.

### 4-[4-Morpholin-4-yl-6-(1-pyridin-4-ylsulfonylcyclopropyl)pyrimidin-2-yl]aniline

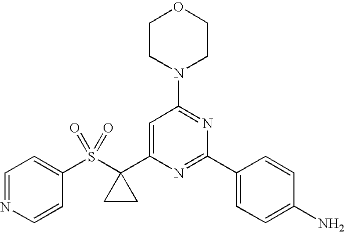

Bis(triphenylphosphine)palladium(II) chloride (0.309 g, 0.44 mmol) was added to 4-(4,4,5,5-tetramethyl-1,3,2-dioxaborolan-2-yl)aniline (2.505 g, 11.43 mmol) and 2-chloro-4-morpholin-4-yl-6-(1-pyridin-4-ylsulfonylcyclopropyl)pyrimidine (3.35 g, 8.80 mmol) and an aqueous solution of sodium carbonate (13.19 mL, 26.39 mmol) in a solvent mixture of DME (20 mL), ethanol (10 mL) and water (10 mL) at RT under an atmosphere of nitrogen. The resulting mixture was stirred at 95° C. for 4 hours. The reaction mixture was diluted with ethyl acetate (20 mL), and washed with water (2×20 mL). The organic layer was dried (MgSO₄), filtered and evaporated to afford crude product. The crude product was purified by flash silica chromatography, elution gradient 0 to 10% methanol in DCM, to give the desired material as a brown solid (2.37 g).

NMR Spectrum: ¹H NMR (400.132 MHz, DMSO-d₆) δ 1.63-1.67 (2H, m), 1.93-1.96 (2H, m), 3.64 (4H, d), 3.67-3.69 (4H, m), 5.52 (2H, s), 6.45 (2H, d), 6.62 (1H, s), 7.47 (2H, d), 7.76 (2H, dd), 8.85 (2H, dd)

LCMS Spectrum: m/z (ESI+) (M+H)+=438; HPLC tR=1.94 min.

### 2-Chloro-4-morpholin-4-yl-6-(1-pyridin-4-ylsulfonylcyclopropyl)pyrimidine

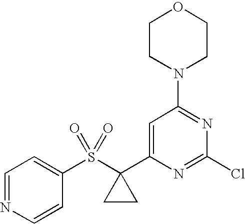

1,2-Dibromoethane (0.510 mL, 22.55 mmol) was added to 2-chloro-4-morpholin-4-yl-6-(pyridin-4-ylsulfonylmethyl)pyrimidine (4 g, 11.27 mmol) in toluene (56.4 mL) followed by tetrabutylammonium bromide (0.363 g, 1.13 mmol) and an aqueous solution of sodium hydroxide (5.64 mL, 56.37 mmol). The reaction was stirred at 60° C. for 7 hours then the toluene removed under reduced pressure and the residue redissolved in DCM. The organics were washed with water, dried (MgSO₄), filtered and evaporated to afford crude product. The crude product was triturated with diethyl ether to give the desired material as a brown solid (3.35 g).

NMR Spectrum: ¹H NMR (400.132 MHz, DMSO-d₆) δ 1.60-1.63 (2H, m), 1.89-1.92 (2H, m), 3.58 (4H, d), 3.63-3.65 (4H, m), 6.81 (1H, s), 7.74 (2H, d), 8.88 (2H, d)

LCMS Spectrum: m/z (ESI+)(M+H)+ 381, HPLC tR=1.70 min

### 2-Chloro-4-morpholin-4-yl-6-(pyridin-4-ylsulfonylmethyl)pyrimidine

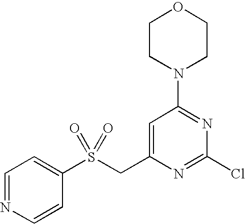

2-Chloro-4-morpholin-4-yl-6-(pyridin-4-ylsulfanylmethyl)pyrimidine (3.28 g, 10.16 mmol) was dissolved in dioxane (45 mL) and 2N sulfuric acid (0.11 mL) was added. The solution was heated to 55° C. A solution of sodium tungstate dihydrate (0.067 g, 0.20 mmol) in water (1.08 mL) was added to the solution and allowed to stir for 10 minutes. Hydrogen peroxide (6.28 mL, 203.2 mmol) was then added dropwise over several minutes. The solution was heated at 55° C. for 3 hours. Water was added and the reaction was allowed to cool. The aqueous solution was extracted with DCM and the organics separated, dried (MgSO₄), filtered and evaporated. The crude product was triturated with diethyl ether to give the desired material as a cream solid (3.20 g).

NMR Spectrum: ¹H NMR (400.132 MHz, DMSO-d₆) δ 3.56 (4H, s), 3.65-3.68 (4H, m), 4.77 (2H, s), 6.84 (1H, s), 7.78 (2H, d), 8.92 (2H, d)

LCMS Spectrum: m/z (ESI+)(M+H)+ 355, HPLC tR=1.53 min

### 2-Chloro-4-morpholin-4-yl-6-(pyridin-4-ylsulfanlmethyl)pyrimidine

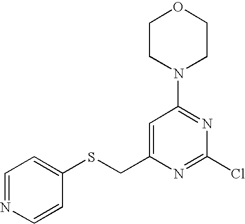

2-Chloro-4-(iodomethyl)-6-morpholin-4-ylpyrimidine (5 g, 14.72 mmol) was added portionwise to 4-mercaptopyridine (1.8 g, 16.20 mmol) and DBU (2.344 mL, 16.20 mmol) in acetonitrile (73.6 mL) at RT. The resulting suspension was stirred at RT for 30 minutes. The reaction mixture was evaporated to dryness, redissolved in DCM (50 mL) and the organics washed with water (50 mL), dried (MgSO₄), filtered and evaporated to afford crude product. The crude product was purified by flash silica chromatography, elution gradient 0 to 100% ethyl acetate in DCM, to give the desired material as a beige solid (3.3 g).

NMR Spectrum: ¹H NMR (400.132 MHz, DMSO-d₆) δ 3.58 (4H, s), 3.64-3.67 (4H, m), 4.24 (2H, s), 6.97 (1H, s), 7.35 (2H, d), 8.38 (2H, d)

LCMS Spectrum: m/z (ESI+)(M+H)+ 323, HPLC tR=1.75 min

The preparation of 2-chloro-4-(iodomethyl)-6-morpholin-4-ylpyrimidine was described earlier.

The preparation of phenyl N-[4-[4-[1-(benzenesulfonyl)cyclopropyl]-6-[(3S)-3-methylmorpholin-4-yl]pyrimidin-2-yl]phenyl]carbamate is described below.

### Phenyl N-[4-[4-[1-(benzenesulfonyl)cyclopropyl]-6-[(3S)-3-methylmorpholin-4-yl]pyrimidin-2-yl]phenyl]carbamate

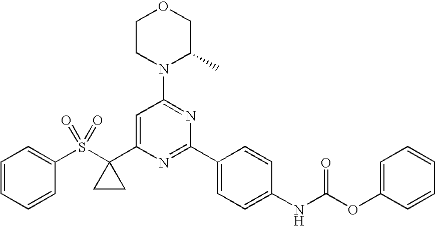

Phenyl chloroformate (0.471 mL, 3.75 mmol) was added dropwise to 4-[4-[1-(benzenesulfonyl)cyclopropyl]-6-[(3S)-3-methylmorpholin-4-yl]pyrimidin-2-yl]aniline (1.690 g, 3.75 mmol) and sodium bicarbonate (0.473 g, 5.63 mmol) in dioxane and the resulting mixture stirred at RT for 18 hours. The solvent was removed, DCM added and the organics washed with water dried (MgSO₄), filtered and evaporated to give the desired material as a beige solid (2.44 g) which was used without further purification.

NMR Spectrum: ¹H NMR (400.132 MHz, DMSO-d6) δ 1.17-1.19 (3H, d), 1.62-1.69 (2H, m), 1.90-1.93 (2H, m), 3.12-3.19 (1H, td), 3.41-3.49 (1H, td), 3.59-3.63 (1H, dd), 3.73-3.76 (1H, d), 3.94-3.98 (1H, dd), 4.10-4.14 (1H, d), 4.39 (1H, bs), 6.66 (1H, s), 7.24-7.30 (3H, m), 7.43-7.47 (2H, t), 7.51-7.53 (2H, d), 7.58-7.61 (2H, t), 7.70-7.74 (1H, t), 7.79-7.81 (2H, d), 7.92-7.94 (2H, d), 10.38 (1H, s).

LCMS Spectrum: m/z (ES+) (M+H)+=571; HPLC tR=3.00 min.

### 4-[4-[1-(Benzenesulfonyl)cyclopropyl]-6-[(3S)-3-methylmorpholin-4-yl]pyrimidin-2-yl]aniline

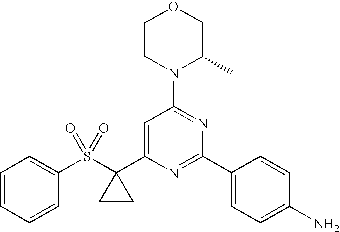

Bis(triphenylphosphine)palladium (II) chloride (0.246 g, 0.35 mmol) was added in one portion to 4-[1-(benzenesulfonyl)cyclopropyl]-2-chloro-6-[(3S)-3-methylmorpholin-4-yl]pyrimidine (2.76 g, 7.01 mmol), 4-(4,4,5,5-tetramethyl-1,3,2-dioxaborolan-2-yl)aniline (1.535 g, 7.01 mmol) and an aqueous solution of sodium carbonate (17.52 mL, 35.04 mmol) in a solvent mixture of 18% DMF, 82% of a 7:3:2 mixture of DME:water:Ethanol) and the resulting mixture stirred at 80° C. for 3 hours under an atmosphere of nitrogen. The crude product was dissolved in ethyl acetate and washed with water. The organics were dried (MgSO₄), filtered and evaporated. The crude product was purified by ion exchange chromatography using an SCX column, eluting with 7M ammonia in methanol, to give the desired material as a yellow solid (3.35 g).

NMR Spectrum: ¹H NMR (400.132 MHz, DMSO-d6) δ 1.15-1.16 (3H, d), 1.58-1.66 (2H, m), 1.85-1.91 (2H, m), 3.07-3.14 (1H, td), 3.41-3.48 (1H, td), 3.58-3.61 (1H, dd), 3.72-3.75 (1H, d), 3.93-3.96 (1H, dd), 4.05-4.08 (1H, d), 4.33 (1H, bs), 5.50 (2H, s), 6.49-6.53 (3H, t), 7.57-7.61 (2H, t), 7.68-7.71 (3H, m), 7.78-7.81 (2H, d).

LCMS Spectrum: m/z (ES+) (M+H)+=45 1; HPLC tR=2.37 min.

### 4-[1-(Benzenesulfonyl)cyclopropyl]-2-chloro-6-[(3S)-3-methylmorpholin-4-yl]pyrimidine

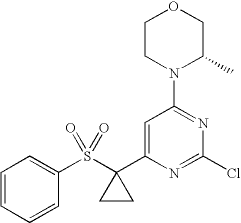

Sodium hydroxide (50% w/w aqueous solution, 299.03 mmol) was added to 4-(benzenesulfonylmethyl)-2-chloro-6-[(3S)-3-methylmorpholin-4-yl]pyrimidine (2.0 g, 5.44 mmol), 1,2-dibromoethane (1.406 mL, 16.31 mmol) and tetrabutylammonium bromide (0.175 g, 0.54 mmol) in toluene (75 mL) and the resulting mixture stirred at 60° C. for 4 hours. Water was added and the mixture was extracted with toluene. The organics were dried (MgSO₄), filtered and evaporated. The crude product was purified by flash silica chromatography, elution gradient 0 to 40% ethyl acetate in DCM, to give the desired material as a white solid (2.76 g).

NMR Spectrum: ¹H NMR (400.132 MHz, DMSO-d₆) δ 1.13-1.15 (3H, d), 1.55-1.57 (2H, m), 1.83-1.86 (2H, m), 3.09-3.16 (1H, td), 3.36-3.43 (1H, td), 3.52-3.56 (1H, dd), 3.68-3.71 (1H, d), 3.86-3.93 (2H, m), 4.20 (1H, bs), 6.67 (1H, s), 7.60-7.63 (2H, m), 7.72-7.77 (3H, m).

LCMS Spectrum: m/z (ES+) (M+H)+=394; HPLC tR=2.28 min.

### 4-(Benzenesulfonylmethyl)-2-chloro-6-[(3S)-3-methylmorpholin-4-yl]pyrimidine

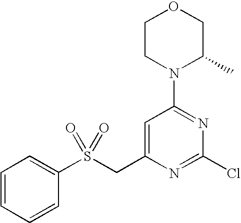

Benzenesulfinic acid, sodium salt (4.22 g, 25.74 mmol) was added to 2-chloro-4-(iodomethyl)-6-[(3S)-3-methylmorpholin-4-yl]pyrimidine (7.0 g, 19.80 mmol) in acetonitrile (200 mL) and the resulting mixture stirred under a nitrogen atmosphere at 80° C. for 20 hours. The reaction was cooled and the solvent was removed. DCM was added and the solution was washed with water. The DCM was dried (MgSO₄), filtered and the solvent was removed. The crude product was purified by flash silica chromatography, elution gradient 0 to 30% ethyl acetate in DCM, to give the desired material as a cream solid (6.21 g).

NMR Spectrum: ¹H NMR (400.132 MHz, DMSO-d₆) δ 1.15-1.16 (3H, d), 3.11-3.18 (1H, td), 3.38-3.45 (1H, td), 3.55-3.58 (1H, dd), 3.70-3.73 (1H, d), 3.85-3.94 (2H, m), 4.15 (1H, bs), 4.64 (2H, s), 6.67 (1H, s), 7.63-7.66 (2H, m), 7.74-7.80 (3H, m).

LCMS Spectrum: m/z (ES+) (M+H)+=368; HPLC tR=2.05 min.

The preparation of 2-chloro-4-(iodomethyl)-6-[(3S)-3-methylmorpholin-4-yl]pyrimidine was described earlier.

The preparation of phenyl N-[4-[4-[1-(benzenesulfonyl)cyclobutyl]-6-[(3S)-3-methylmorpholin-4-yl]pyrimidin-2-yl]phenyl]carbamate is described below.

### Phenyl N-[4-[4-[1-(benzenesulfonyl)cyclobutyl]-6-[(3S)-3-methylmorpholin-4-yl]pyrimidin-2-yl]phenyl]carbamate

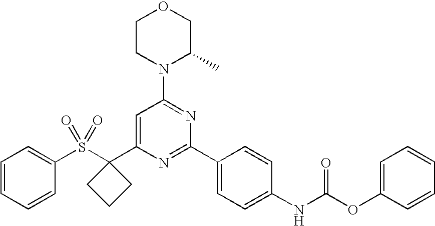

Phenyl chloroformate (0.083 mL, 0.66 mmol) was added to 4-[4-[1-(benzenesulfonyl)cyclobutyl]-6-[(3S)-3-methylmorpholin-4-yl]pyrimidin-2-yl]aniline (0.307 g, 0.66 mmol) and sodium hydrogen carbonate (0.083 g, 0.99 mmol) in dioxane and the resulting mixture was stirred at RT for 2 hours. The solvent was removed and the residue partitioned between DCM and water. The organics were washed with water, dried (MgSO₄), filtered and evaporated to give the desired material as a gum (0.386 g) which was used without further purification.

NMR Spectrum: ¹H NMR (400.132 MHz, CDCl₃) δ 1.31-1.33 (3H, d), 1.87-1.99 (1H, m), 2.20-2.31 (1H, m), 2.75-2.83 (2H, m), 3.17-3.32 (3H, m), 3.57-3.63 (1H, td), 3.73-3.77 (1H, dd), 3.80-3.83 (1H, d), 4.01-4.05 (1H, dd), 4.09-4.12 (1H, d), 4.44 (1H, bs), 6.55 (1H, s), 6.78-6.81 (1H, d), 7.16-7.28 (4H, m), 7.36-7.42 (5H, m), 7.49-7.51 (2H, d), 7.56 (1H, s), 7.89-7.92 (2H, d).

LCMS Spectrum: m/z (ES+) (M+H)+=585; HPLC tR=3.30 min.

### 4-[4-[1-(Benzenesulfonyl)cyclobutyl]-6-[(3S)-3-methylmorpholin-4-yl]pyrimidin-2-yl]aniline

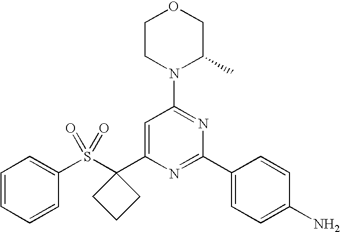

Bis(triphenylphosphine)palladium (II) chloride (0.023 g, 0.03 mmol) was added in one portion to 4-[1-(benzenesulfonyl)cyclobutyl]-2-chloro-6-[(3S)-3-methylmorpholin-4-yl]pyrimidine (0.270 g, 0.66 mmol), 4-(4,4,5,5-tetramethyl-1,3,2-dioxaborolan-2-yl)aniline (0.145 g, 0.66 mmol) and an aqueous solution of sodium carbonate (1.653 mL, 3.31 mmol) in a solvent mixture comprising 18% DMF, 82% of a 7:3:2 mixture of DME:water:Ethanol and the resulting mixture was stirred at 80° C. for 3 hours under an atmosphere of nitrogen. The crude product was purified by ion exchange chromatography using an SCX column, eluting with 7M ammonia in methanol, to give a sample which was further purified by flash silica chromatography, elution gradient 0 to 40% ethyl acetate in DCM, to give the desired as a colourless gum (0.395 g).

NMR Spectrum: ¹H NMR (400.132 MHz, CDCl₃) δ 1.30-1.32 (3H, d), 1.87-1.98 (1H, m), 2.19-2.29 (1H, m), 2.74-2.83 (2H, m), 3.14-3.22 (2H, m), 3.24-3.31 (1H, td), 3.57-3.63 (1H, td), 3.73-3.85 (4H, m), 4.01-4.04 (1H, dd), 4.08-4.12 (1H, d), 4.43-4.45 (1H, m), 6.48 (1H, s), 6.55-6.57 (2H, d), 7.26-7.31 (2H, t), 7.39-7.43 (1H, t), 7.48-7.51 (2H, dd), 7.74-7.76 (2H, d).

LCMS Spectrum: m/z (ES+) (M+H)+=465; HPLC tR=2.49 min.

### 4-[1-(Benzenesulfonyl)cyclobutyl]-2-chloro-6-[(3S)-3-methylmorpholin-4-yl]pyrimidine

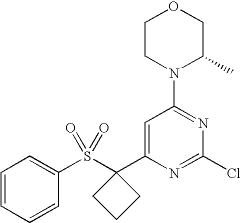

Sodium hydroxide (50% w/w aqueous solution, 120.21 mmol) was added to 4-(benzenesulfonylmethyl)-2-chloro-6-[(3S)-3-methylmorpholin-4-yl]pyrimidine (0.804 g, 2.19 mmol), 1,3-dibromopropane (0.666 mL, 6.56 mmol) and tetrabutylammonium bromide (0.070 g, 0.22 mmol) in toluene (50 mL) and the resulting mixture was stirred at 45° C. for 1 hour. Water was added, the organics separated, washed with water, dried (MgSO₄), filtered and evaporated. The crude product was purified by flash silica chromatography, elution gradient 0 to 40% ethyl acetate in DCM, to give the desired material as a white solid (0.27 g).

NMR Spectrum: ¹H NMR (400.132 MHz, CDCl₃) δ 1.29-1.31 (3H, d), 1.89-1.96 (1H, m), 2.17-2.28 (1H, m), 2.63-2.67 (2H, m), 3.07-3.16 (2H, m), 3.22-3.29 (1H, td), 3.50-3.57 (1H, td), 3.67-3.70 (1H, dd), 3.76-3.79 (1H, d), 3.95-4.01 (2H, m), 4.28 (1H, bs), 6.52 (1H, s), 7.41-7.44 (2H, t), 7.48-7.50 (2H, m), 7.57-7.61 (1H, m).

LCMS Spectrum: m/z (ES+) (M+H)+=408; HPLC tR=2.35 min.

The preparation of 4-(benzenesulfonylmethyl)-2-chloro-6-[(3S)-3-methylmorpholin-4-yl]pyrimidine was described earlier.

The preparation of phenyl N-[4-[4-[1-(benzenesulfonyl)cyclopentyl]-6-[(3S)-3-methylmorpholin-4-yl]pyrimidin-2-yl]phenyl]carbamate is described below.

Phenyl N-[4-[4-[1-(benzenesulfonyl)cyclopentyl]-6-[(3S)-3-methylmorpholin-4-yl]pyrimidin-2-yl]phenyl]carbamate

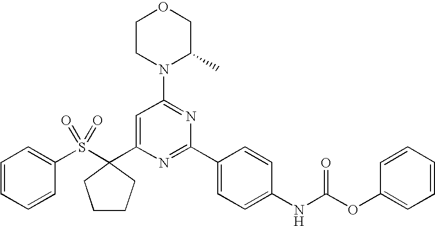

Phenyl chloroformate (0.244 mL, 1.94 mmol) was added to 4-[4-[1-(benzenesulfonyl)cyclopentyl]-6-[(3S)-3-methylmorpholin-4-yl]pyrimidin-2-yl]aniline (0.930 g, 1.94 mmol) and sodium hydrogen carbonate (0.245 g, 2.91 mmol) in dioxane and the resultant mixture stirred at RT for 2 hours. The solvent was removed and the residue partitioned between DCM and water. The organics were washed with water, dried (MgSO₄), filtered and evaporated to give the desired material as a beige solid (1.19 g).

NMR Spectrum: ¹H NMR (400.132 MHz, DMSO-d₆) δ 1.21-1.23 (3H, d), 1.52-1.56 (2H, m), 1.80-1.88 (2H, m), 2.55-2.60 (2H, m), 2.67-2.69 (2H, m), 3.12-3.19 (1H, td), 3.46-3.52 (1H, td), 3.63-3.67 (1H, dd), 3.75-3.78 (1H, d), 3.95-3.99 (1H, dd), 4.12-4.16 (1H, d), 4.50 (1H, bs), 6.66 (1H, s), 7.23-7.30 (3H, m), 7.42-7.51 (8H, m), 7.56-7.60 (1H, m), 7.88-7.90 (2H, d), 10.37 (1H, s).

LCMS Spectrum: m/z (ES+) (M+H)+=599; HPLC tR=3.44 min.

### 4-[4-[1-(Benzenesulfonyl)cyclopentyl]-6-[(3S)-3-methylmorpholin-4-yl]pyrimidin-2-yl]aniline

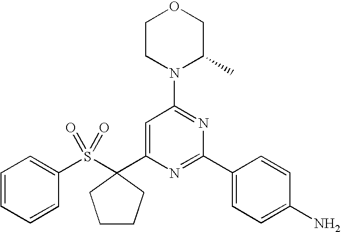

Bis(triphenylphosphine)palladium (II) chloride (0.072 g, 0.10 mmol) was added in one portion to 4-[1-(benzenesulfonyl)cyclopentyl]-2-chloro-6-[(3S)-3-methylmorpholin-4-yl]pyrimidine (0.863 g, 2.05 mmol), 4-(4,4,5,5-tetramethyl-1,3,2-dioxaborolan-2-yl)aniline (0.448 g, 2.05 mmol) and an aqueous solution of sodium carbonate (5.11 mL, 10.23 mmol) in a solvent mixture comprising 18% DMF, 82% of a 7:3:2 mixture of DME:water:Ethanol and the resultant mixture stirred at 80° C. for 3 hours. The crude product was purified by ion exchange chromatography using an SCX column, eluting with 7M ammonia in methanol, to give the desired material as a beige solid (0.93 g).

NMR Spectrum: ¹H NMR (400.132 MHz, DMSO-d₆) δ 1.19-1.20 (3H, d), 1.52-1.55 (2H, m), 1.81-1.85 (2H, m), 2.50 (2H, m), 2.67-2.70 (2H, m), 3.07-3.15 (1H, td), 3.45-3.51 (1H, td), 3.62-3.65 (1H, dd), 3.74-3.77 (1H, d), 3.94-3.97 (1H, dd), 4.04-4.08 (1H, d), 4.43-4.45 (1H, bs), 5.47 (2H, s), 6.48-6.51 (3H, m), 7.42-7.48 (4H, m), 7.57-7.62 (1H, m), 7.65-7.67 (2H, d).

LCMS Spectrum: m/z (ES+) (M+H)+=479; HPLC tR=2.67 min.

### 4-[1-(Benzenesulfonyl)cyclopentyl]-2-chloro-6-[(3S)-3-methylmorpholin-4-yl]pyrimidine

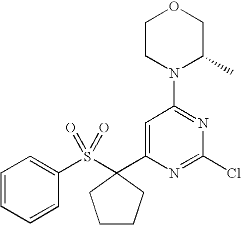

Sodium hydroxide (50% w/w aqueous solution, 112.53 mmol) was added to 4-(benzenesulfonylmethyl)-2-chloro-6-[(3S)-3-methylmorpholin-4-yl]pyrimidine (0.753 g, 2.05 mmol), 1,4-dibromobutane (0.733 mL, 6.14 mmol) and tetrabutylammonium bromide (0.066 g, 0.20 mmol) in toluene (50 mL) and the resulting suspension stirred at 60° C. for 4 hours. Water was added, the organic layer separated and washed twice with water. The organics were dried (MgSO₄), filtered and evaporated. The crude product was purified by flash silica chromatography, elution gradient 0 to 40% ethyl acetate in DCM, to give the desired material as a colourless gum (0.904 g).

NMR Spectrum: ¹H NMR (400.132 MHz, CDCl₃) δ 1.28-1.30 (3H, d), 1.52-1.63 (2H, m), 1.83-1.93 (2H, m), 2.41-2.49 (2H, m), 2.56-2.66 (2H, m), 3.20-3.28 (1H, td), 3.49-3.56 (1H, td), 3.65-3.69 (1H, dd), 3.74-3.77 (1H, d), 3.95-3.99 (2H, m), 4.27 (1H, bs), 6.69 (1H, s), 7.38-7.46 (4H, m), 7.54-7.58 (1H, m).

LCMS Spectrum: m/z (ES+) (M+H)+=422; HPLC tR=2.61 min.

The preparation of 4-(benzenesulfonylmethyl)-2-chloro-6-[(3S)-3-methylmorpholin-4-yl]pyrimidine was described earlier.

The preparation of phenyl N-[4-[4-[(3S)-3-methylmorpholin-4-yl]-6-[4-[3-tri(propan-2-yl)silyloxypropylsulfonyl]oxan-4-yl]pyrimidin-2-yl]phenyl]carbamate is described below.

### Phenyl N-[4-[4-[(3S)-3-methylmorpholin-4-yl]-6-[4-[3-tri(propan-2-yl)silyloxypropylsulfonyl]oxan-4-yl]pyrimidin-2-yl]phenyl]carbamate

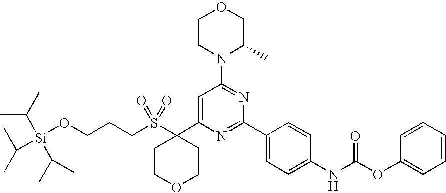

Sodium bicarbonate (0.380 g, 4.53 mmol) was added to 4-[4-[(3S)-3-methylmorpholin-4-yl]-6-[4-[3-tri(propan-2-yl)silyloxypropylsulfonyl]oxan-4-yl]pyrimidin-2-yl]aniline (1.910 g, 3.02 mmol), in 1,4-dioxane (15.09 mL) at RT. Phenyl chloroformate (0.380 mL, 3.02 mmol) was added dropwise over 2 minutes and the reaction stirred at RT for 2 hours. The reaction mixture was evaporated to dryness and redissolved in DCM (40 mL), and washed with water (40 mL), The organic layer was dried (MgSO₄), filtered and evaporated to afford a solid which was triturated with diethyl ether to give the desired material as an amber solid (2.25 g).

NMR Spectrum: ¹H NMR (400.132 MHz, DMSO-d₆) δ 0.87-0.88 (18H, d), 1.09-1.13 (1H, t), 1.23-1.25 (3H, d), 1.31 (1H, s), 1.74-1.81 (2H, qu), 2.20-2.27 (2H, td), 2.83-2.88 (2H, t), 3.00-3.04 (2H, td), 3.18-3.29 (3H, m), 3.49-3.51 (2H, m), 3.63-3.66 (2H, t), 3.78-3.80 (1H, d), 3.91-3.97 (2H, qu), 3.99-4.03 (1H, dd), 4.30-4.33 (1H, d), 4.58 (1H, exchange), 6.90 (1H, s), 7.25-7.27 (2H, dd), 7.29-7.31 (1H, dd), 7.44-7.47 (2H, d), 7.62-7.64 (2H, d), 8.30-8.32 (2H, d)

LCMS Spectrum: m/z (ESI+) (M+H)+=753.4; HPLC tR=3.97 min.

### 4-[4-[(3S)-3-Methylmorpholin-4-yl]-6-[4-[3-tri(propan-2-yl)silyloxypropylsulfonyl]oxan-4-yl]pyrimidin-2-yl]aniline

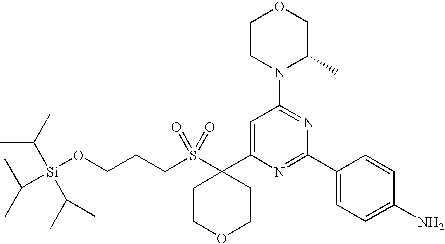

Dichlorobis(triphenylphosphine)palladium (II) (0.118 g, 0.17 mmol) was added to 3-[4-[2-chloro-6-[(3S)-3-methylmorpholin-4-yl]pyrimidin-4-yl]oxan-4-yl]sulfonylpropoxy-tri(propan-2-yl)silane (1.94 g, 3.37 mmol), 4-(4,4,5,5-tetramethyl-1,3,2-dioxaborolan-2-yl)aniline (0.959 g, 4.38 mmol) and 2M aqueous sodium carbonate (6.06 mL, 12.12 mmol) in water (9.35 mL), ethanol (3.74 mL) and DME (3.74 mL) and the resulting solution stirred at 80° C. for 3 hours under a nitrogen atmosphere. The reaction mixture was diluted with ethyl acetate (100 mL), and washed sequentially with water (100 mL) and saturated brine (100 mL). The organic layer was dried (MgSO₄), filtered and evaporated. The crude product was purified by flash silica chromatography, elution gradient 0 to 75% ethyl acetate in DCM, to give the desired material as an amber solid (1.91 g).

NMR Spectrum: ¹H NMR (400.132 MHz, DMSO-d₆) δ 0.90-0.91 (18H, d), 1.21-1.23 (3H, d), 1.73-1.80 (2H, qu), 2.17-2.25 (2H, td), 2.79-2.84 (2H, t), 2.98-3.02 (2H, td), 3.28 (2H, s), 3.31 (2H, s), 3.47-3.53 (1H, td), 3.63-3.67 (3H, m), 3.76-3.79 (1H, d), 3.90-3.96 (2H, qu), 3.97-4.01 (1H, dd), 4.25-4.29 (1H, d), 4.53-4.54 (1H, m), 5.53-5.55 (1H, d), 6.59-6.61 (2H, d), 6.76 (1H, s), 8.02-8.05 (2H, d)

LCMS Spectrum: m/z (ESI+) (M+H)+=633.34; HPLC tR=3.67 min.

### 3-[4-[2-Chloro-6-[(3S)-3-methylmorpholin-4-yl]pyrimidin-4-yl]oxan-4-yl]sulfonylpropoxy-tri(propan-2-yl)silane

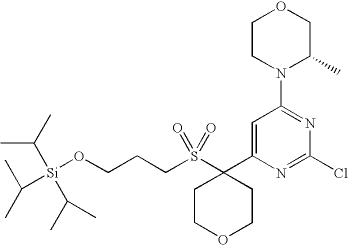

Sodium tert-butoxide (1.519 g, 15.80 mmol) was added portionwise to 3-[[2-chloro-6-[(3S)-3-methylmorpholin-4-yl]pyrimidin-4-yl]methylsulfonyl]propoxy-tri(propan-2-yl)silane (2 g, 3.95 mmol) and bis(2-bromoethyl)ether (1.987 mL, 15.80 mmol) in DMF (19.76 mL) at RT over a period of 5 minutes under a nitrogen atmosphere. The resulting solution was stirred at RT for 16 hours. The reaction mixture was quenched with saturated aqueous ammonium chloride solution (0.5 mL), concentrated and diluted with ethyl acetate (200 mL). The organics were separated, washed sequentially with water (2×200 mL) and saturated brine (100 mL), dried (MgSO₄), filtered and evaporated. The crude product was purified by flash silica chromatography, elution gradient 40 to 60% ethyl acetate in isohexane, to give the desired material as an off white solid (1.94 g).

NMR Spectrum: ¹H NMR (400.132 MHz, DMSO-d₆) δ 0.98-1.02 (18H, m), 1.20-1.22 (4H, q), 1.78-1.85 (2H, qu), 2.13-2.20 (2H, t), 2.65-2.69 (2H, m), 2.95-3.03 (2H, m), 3.15-3.24 (3H, q), 3.28-3.31 (3H, d), 3.42-3.49 (1H, td), 3.59-3.63 (1H, dd), 3.72-3.76 (2H, q), 3.87-3.92 (2H, qu), 3.94-3.98 (1H, dd), 4.11-4.14 (1H, d), 4.45 (1H, exchange), 6.99 (1H, s)

LCMS Spectrum: m/z (ESI+)(M+H)+ 576.31; HPLC tR=4.06 min

The preparation of 3-[[2-chloro-6-[(3S)-3-methylmorpholin-4-yl]pyrimidin-4-yl]methylsulfonyl]propoxy-tri(propan-2-yl)silane was described earlier.

The preparation of phenyl N-[4-[4-[(3S)-3-methylmorpholin-4-yl]-6-[1-[3-tri(propan-2-yl)silyloxypropylsulfonyl]cyclopropyl]pyrimidin-2-yl]phenyl]carbamate is described below.

### Phenyl N-[4-[4-[(3S)-3-methylmorpholin-4-yl]-6-[1-[3-tri(propan-2-yl)silyloxypropylsulfonyl]cyclopropyl]pyrimidin-2-yl]phenyl]carbamate

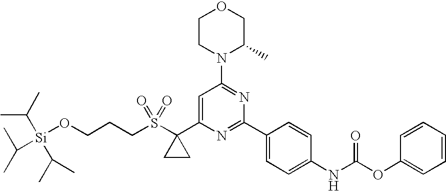

Sodium bicarbonate (0.749 g, 8.92 mmol) was added to 4-[4-[(3S)-3-methylmorpholin-4-yl]-6-[1-[3-tri(propan-2-yl)silyloxypropylsulfonyl]cyclopropyl]pyrimidin-2-yl]aniline (3.5 g, 5.94 mmol), in 1,4-dioxane (29.7 mL) at RT. Phenyl chloroformate (0.748 mL, 5.94 mmol) was added dropwise over 2 minutes and the reaction stirred at RT for 2 hours. The reaction mixture was evaporated to dryness, redissolved in DCM (100 mL), and washed with water (100 mL). The organic layer was dried (MgSO₄), filtered and evaporated to afford a solid, which was triturated with diethyl to give the desired material as a light yellow solid (4.13 g).

NMR Spectrum: ¹H NMR (400.132 MHz, DMSO-d₆) δ 0.95-0.96 (18H, d), 1.24-1.26 (3H, d), 1.30 (2H, s), 1.58-1.60 (2H, m), 1.54-1.67 (2H, m), 1.95-2.02 (2H, sex), 3.18-3.26 (1H, td), 3.38-3.43 (2H, m), 3.46-3.49 (3H, m), 3.62-3.66 (1H, dd), 3.75-3.78 (2H, t), 3.97-4.01 (1H, dd), 4.21-4.24 (1H, d), 4.57 (1H, exchange), 6.86 (1H, s), 7.25-7.27 (2H, d), 7.29-7.31 (1H, d), 7.44-7.46 (2H, d), 7.63-7.65 (2H, d), 8.27-8.30 (2H, d), 10.45 (1H, exchange)

LCMS Spectrum: m/z (ESI+)(M+H)+ 709.41; HPLC tR=3.82 min

### 4-[4-[(3S)-3-Methylmorpholin-4-yl]-6-[1-[3-tri(propan-2-yl)silyloxypropylsulfonyl]cyclopropyl]pyrimidin-2-yl]aniline

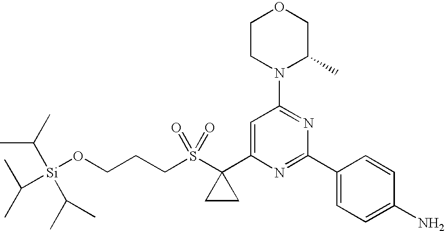

Dichlorobis(triphenylphosphine)palladium (II) (0.214 g, 0.31 mmol) was added to 3-[1-[2-chloro-6-[(3S)-3-methylmorpholin-4-yl]pyrimidin-4-yl]cyclopropyl]sulfonylpropoxy-tri(propan-2-yl)silane (3.25 g, 6.11 mmol), 4-(4,4,5,5-tetramethyl-1,3,2-dioxaborolan-2-yl)aniline (1.739 g, 7.94 mmol) and 2M aqueous sodium carbonate (10.99 mL, 21.98 mmol) in water (16.96 mL), ethanol (6.79 mL) and DME (6.79 mL) and the resulting solution stirred at 80° C. for 3 hours under a nitrogen atmosphere. The reaction mixture was diluted with ethyl acetate (100 mL), and washed sequentially with water (100 mL) and saturated brine (100 mL). The organic layer was dried (MgSO₄), filtered and evaporated. The crude product was purified by flash silica chromatography, elution gradient 0 to 75% ethyl acetate in DCM, to give the desired material as an amber gum, which solidified on standing (3.50 g).

NMR Spectrum: ¹H NMR (400.132 MHz, DMSO-d₆) δ 0.98-0.99 (18H, d), 1.53-1.58 (2H, m), 1.60-1.64 (2H, m), 1.96-2.03 (4H, m), 3.14-3.22 (1H, td), 3.29-3.31 (1H, d), 3.45-3.49 (3H, m), 3.61-3.64 (1H, dd), 3.75-3.78 (3H, t), 3.96-4.00 (1H, dd), 4.02-4.08 (1H, q), 4.15-4.19 (1H, d), 4.51 (1H, m), 5.55-5.57 (1H, d), 6.59-6.61 (2H, d), 6.71 (1H, s), 8.02-8.04 (2H, d)

LCMS Spectrum: m/z (ESI+)(M+H)+ 589.88; HPLC tR=3.80 min

The preparation of 3-[1-[2-chloro-6-[(3S)-3-methylmorpholin-4-yl]pyrimidin-4-yl]cyclopropyl]sulfonylpropoxy-tri(propan-2-yl)silane was described earlier.

The preparation of phenyl N-[4-[4-[4-(benzenesulfonyl)oxan-4-yl]-6-[(3S)-3-methylmorpholin-4-yl]pyrimidin-2-yl]phenyl]carbamate is described below.

### Phenyl N-[4-[4-[4-(benzenesulfonyl)oxan-4-yl]-6-[(3S)-3-methylmorpholin-4-yl]pyrimidin-2-yl]phenyl]carbamate

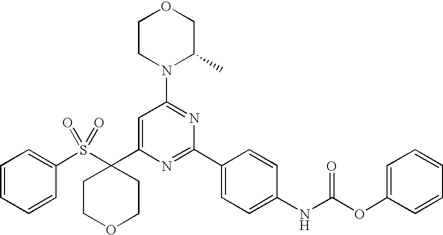

Phenyl chloroformate (0.098 mL, 0.78 mmol) was added dropwise to 4-[4-[4-(benzenesulfonyl)oxan-4-yl]-6-[(3S)-3-methylmorpholin-4-yl]pyrimidin-2-yl]aniline (0.35 g, 0.71 mmol) and sodium bicarbonate (0.089 g, 1.06 mmol) in dioxane (25 mL) and the resulting suspension stirred at RT for 18 hours. The reaction mixture was diluted with DCM (20 mL), and washed with water (20 mL), the organic layer dried (MgSO₄), filtered and evaporated to give the desired material as an orange gum (0.45 g).

NMR Spectrum: ¹H NMR (400.132 MHz, DMSO-d₆) δ 1.20 (3H, d), 2.20-2.30 (2H, td), 2.75 (2H, m), 3.23 (3H, m), 3.50 (1H, td), 3.65 (1H, dd), 3.75 (1H, d), 3.85 (2H, dd), 3.97 (1H, dd), 4.20 (1H, d), 4.55 (1H, br s), 6.70-6.80 (3H, m), 7.15 (1H, t), 7.25 (2H, dd), 7.40-7.55 (7H, m), 7.85 (2H, d), 10.40 (1H, s)

LCMS Spectrum: m/z (ESI+) (M+H)+=615; HPLC tR=2.93 min.

### 4-[4-[4-(Benzenesulfonyl)oxan-4-yl]-6-[(3S)-3-methylmorpholin-4-yl]pyrimidin-2-yl]aniline

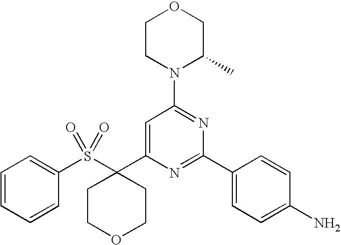

Bis(triphenylphosphine)palladium(II) chloride (0.067 g, 0.09 mmol) was added in one portion to 4-[4-(benzenesulfonyl)oxan-4-yl]-2-chloro-6-[(3S)-3-methylmorpholin-4-yl]pyrimidine (0.83 g, 1.90 mmol), 4-(4,4,5,5-tetramethyl-1,3,2-dioxaborolan-2-yl)aniline (0.498 g, 2.27 mmol) and an aqueous solution of sodium carbonate (4.74 mL, 9.48 mmol) in a DMF (3.60 mL), DME (9.56 mL), water (4.1 mL) and ethanol (2.72 mL) solution mixture. The resulting solution was stirred at 80° C. for 3 hours under a nitrogen atmosphere. The reaction mixture was evaporated to dryness, redissolved in ethyl acetate (50 mL), and washed sequentially with water (20 mL) and saturated brine (20 mL). The organic layer was dried (MgSO₄), filtered and evaporated to afford crude product. The crude product was purified by flash silica chromatography, elution gradient 0 to 20% ethyl acetate in DCM, to give the desired material as a beige solid (0.82 g).

NMR Spectrum: ¹H NMR (400.132 MHz, DMSO-d₆) δ 1.20 (3H, d), 2.20-2.30 (2H, td), 2.75 (2H, m), 3.10-3.20 (3H, m), 3.50 (1H, td), 3.65 (1H, dd), 3.75 (1H, d), 3.85 (2H, dd), 3.97 (1H, dd), 4.20 (1H, d), 4.50 (1H, d), 5.47 (2H, s), 6.45 (2H, d), 6.60 (1H, s), 7.40 (4H, m), 7.60 (3H, m)

LCMS Spectrum: m/z (ESI+) (M+H)+=495; HPLC tR=2.37 min.

### 4-[4-(Benzenesulfonyl)oxan-4-yl]-2-chloro-6-[(3S)-3-methylmorpholin-4-yl]pyrimidine

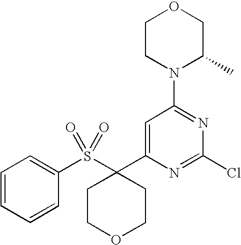

50% v/v aqueous sodium hydroxide (4.49 g, 112.14 mmol) was added to 4-(benzenesulfonylmethyl)-2-chloro-6-[(3S)-3-methylmorpholin-4-yl]pyrimidine (0.75 g, 2.04 mmol), tetrabutylammonium bromide (0.066 g, 0.20 mmol) and 1-bromo-2-(2-bromoethoxy)ethane (1.419 g, 6.12 mmol) in toluene (50 mL). The resulting mixture was stirred at 60° C. for 6 hours. The reaction mixture was diluted with water (50 mL), and washed twice with more water (25 mL). The organic layer was dried (MgSO₄), filtered and evaporated. The crude product was purified by flash silica chromatography, elution gradient 0 to 20% ethyl acetate in DCM, to give the desired material as a white solid (0.83 g).

NMR Spectrum: ¹H NMR (400.132 MHz, DMSO-d₆) δ 1.15 (3H, d), 2.10-2.23 (2H, td), 2.60 (2H, td), 3.10-3.20 (3H, m), 3.25 (1H, d), 3.40 (1H, td), 3.60 (1H, dd), 3.70 (1H, dd), 3.80 (2H, dd), 3.90 (1H, dd), 4.40 (1H, d), 6.70 (1H, s), 7.40 (2H, d), 7.60 (2H, td), 7.75 (1H, td)

LCMS Spectrum: m/z (ESI+) (M+H)+=438; HPLC tR=2.45 min.

The preparation of 4-(benzenesulfonylmethyl)-2-chloro-6-[(3S)-3-methylmorpholin-4-yl]pyrimidine was described earlier.

The preparation of phenyl N-[4-[4-[1-(benzenesulfonyl)cyclopropyl]-6-morpholin-4-ylpyrimidin-2-yl]phenyl]carbamate is described below.

### Phenyl N-[4-[4-[1-(benzenesulfonyl)cyclopropyl]-6-morpholin-4-ylpyrimidin-2-yl]phenyl]carbamate

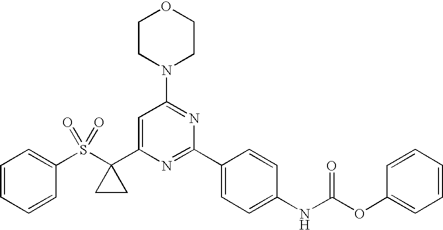

Phenyl chloroformate (0.307 mL, 2.45 mmol) was added to 4-[4-[1-(benzenesulfonyl)cyclopropyl]-6-morpholin-4-ylpyrimidin-2-yl]aniline (1.069 g, 2.45 mmol) and sodium hydrogen carbonate (0.309 g, 3.67 mmol) in dioxane and the resulting suspension stirred at RT overnight. The solids were filtered and washed with dioxane and water and then dried in the vacuum oven at 50° C. overnight. The filtrate was concentrated and the solids were filtered, rinsed with water and dried in the vacuum oven overnight. The two crops were combined to give the desired material as a beige solid (1.132 g).

NMR Spectrum: ¹H NMR (400.132 MHz, DMSO-d₆) δ 1.62-1.65 (2H, q), 1.89-1.92 (2H, q), 3.66-3.70 (8H, m), 6.71 (1H, s), 7.24-7.31 (3H, m), 7.43-7.47 (2H, t), 7.51-7.54 (2H, d), 7.58-7.62 (2H, t), 7.70-7.74 (1H, tt), 7.79-7.81 (2H, d), 7.90-7.92 (2H, d), 10.40 (1H, bs).

LCMS Spectrum: m/z (ES+) (M+H)+=557; HPLC tR=3.03 min.

### 4-[4-[1-(Benzenesulfonyl)cyclopropyl]-6-morpholin-4-ylpyrimidin-2-yl]aniline

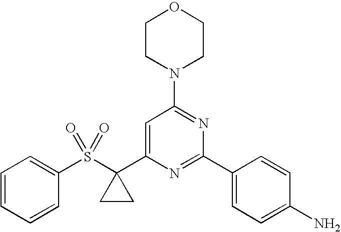

Bis(triphenylphosphine)palladium (II) chloride (0.088 g, 0.13 mmol) was added in one portion to 4-[1-(benzenesulfonyl)cyclopropyl]-2-chloro-6-morpholin-4-ylpyrimidine (0.956 g, 2.52 mmol), 4-(4,4,5,5-tetramethyl-1,3,2-dioxaborolan-2-yl)aniline (0.552 g, 2.52 mmol) and an aqueous solution of sodium carbonate (6.29 mL, 12.59 mmol) in a solvent mixture comprising 18% DMF and 82% of a 7:3:2 mixture of DME:water:Ethanol and the solution stirred at 80° C. for 3 hours under a nitrogen atmosphere. The crude product was purified by ion exchange chromatography using an SCX column, eluting with 7M ammonia in methanol to give a sample that was further purified by flash silica chromatography, elution gradient 0 to 40% ethyl acetate in DCM, to give the desired material as a white solid (1.07 g).

NMR Spectrum: ¹H NMR (400.132 MHz, DMSO-d₆) δ 1.58-1.62 (2H, q), 1.86-1.89 (2H, q), 3.59-3.60 (4H, m), 3.67-3.69 (4H, m), 5.50 (2H, s), 6.48-6.50 (2H, d), 6.58 (1H, s), 7.57-7.61 (2H, t), 7.66-7.68 (2H, d), 7.69-7.73 (1H, tt), 7.78-7.80 (2H, m).

LCMS Spectrum: m/z (ES+) (M+H)+=437; HPLC tR=2.19 min.

### 4-[1-(Benzenesulfonyl)cyclopropyl]-2-chloro-6-morpholin-4-yl]pyrimidine

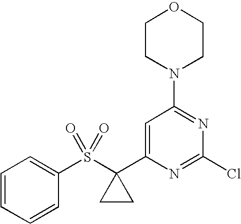

Sodium hydroxide (50% w/w aqueous solution, 248.52 mmol) was added 4-(benzenesulfonylmethyl)-2-chloro-6-morpholin-4-ylpyrimidine (1.599 g, 4.52 mmol), 1,2-dibromoethane (1.168 mL, 13.56 mmol) and tetrabutylammonium bromide (0.146 g, 0.45 mmol) in toluene (75 mL) and the resulting suspension stirred at 60° C. for 18 hours. Water was added to the mixture, the organics separated, washed twice with water, dried (MgSO₄), filtered and evaporated. The crude product was purified by flash silica chromatography, elution gradient 0 to 40% ethyl acetate in DCM, to give the desired material as a white solid (0.956 g).

NMR Spectrum: ¹H NMR (400.132 MHz, DMSO-d₆) δ 1.53-1.56 (2H, q), 1.82-1.85 (2H, q), 3.53 (4H, bs), 3.63-3.65 (4H, t), 6.72 (1H, s), 7.59-7.63 (2H, m), 7.73-7.77 (3H, m).

LCMS Spectrum: m/z (ES+) (M+H)+=380; HPLC tR=2.02 min.

### 4-(Benzenesulfonylmethyl)-2-chloro-6-morpholin-4-ylpyrimidine

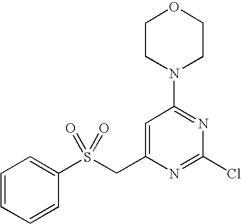

Sodium benzenesulfinate (4.31 g, 26.26 mmol) was added to 2-chloro-4-(iodomethyl)-6-morpholin-4-ylpyrimidine (6.86 g, 20.20 mmol) in acetonitrile (200 mL) at 22° C. under nitrogen. The resulting slurry was stirred at 80° C. for 3 hours. The reaction had gone to completion. The solvent was removed and DCM and water were added. The DCM was washed with water, dried over MgSO₄, filtered and evaporated. The crude product was purified by flash silica chromatography, elution gradient 0 to 30% ethyl acetate in DCM. Pure fractions were evaporated to dryness to afford 4-(2-chloro-6-(phenylsulfonylmethyl)pyrimidin-4-yl)morpholine (4.96 g, 69.4%) as a cream solid.

NMR Spectrum: ¹H NMR (400.132 MHz, DMSO-d₆) δ 3.53 (4H, bs), 3.64-3.67 (4H, t), 4.61 (2H, s), 6.71 (1H, s), 7.63-7.67 (2H, m), 7.75-7.81 (3H, m).

LCMS Spectrum: m/z (ES+) (M+H)+=354; HPLC tR=1.79 min.

The preparation of 2-chloro-4-(iodomethyl)-6-morpholin-4-ylpyrimidine was described earlier.

The preparation of phenyl N-[4-[4-[(3 S)-3-methylmorpholin-4-yl]-6-[1-[(4-methyl-1,3-thiazol-2-yl)sulfonyl]cyclopentyl]pyrimidin-2-yl]phenyl]carbamate is described below:

### Phenyl N-[4-[4-[(3S)-3-methylmorpholin-4-yl]-6-[1-[(4-methyl-1,3-thiazol-2- yl)sulfonyl]cyclopentyl]pyrimidin-2-yl]phenyl]carbamate

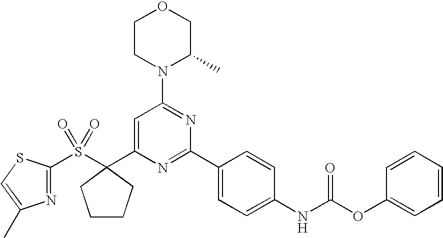

Phenylchloroformate (0.232 mL, 1.85 mmol) was added to 4-[4-[(3S)-3-methylmorpholin-4-yl]-6-[1-[(4-methyl-1,3-thiazol-2-yl)sulfonyl]cyclopentyl]pyrimidin-2-yl]aniline (840 mg, 1.68 mmol) and sodium bicarbonate (212 mg, 2.52 mmol) in dioxane (50 mL) at 10° C. under a nitrogen atmosphere. The resulting mixture was stirred at 10° C. for 2 hours. The reaction mixture was diluted with ethyl acetate (100 mL), and washed sequentially with water (100 mL) and saturated brine (100 mL). The organic layer was dried (MgSO₄), filtered and evaporated to afford Phenyl N-[4-[4-[(3S)-3-methylmorpholin-4-yl]-6-[1-[(4-methyl-1,3-thiazol-2-yl)sulfonyl]cyclopentyl]pyrimidin-2-yl]phenyl]carbamate (1.24 g) as a yellow dry film.

NMR Spectrum: ¹H NMR (400.132 MHz, DMSO-d₆) δ 1.22 (3H, d), 1.60-1.63 (2H, m), 1.83-1.92 (2H, m), 2.38 (3H, s), 2.76-2.80 (4H, m), 3.12-3.19 (1H, m), 3.49-3.55 (1H, m), 3.61-3.66 (1H, m), 3.76 (1H, d), 3.95-3.99 (1H, m), 4.15-4.18 (1H, m), 4.49-4.56 (1H, m), 6.70 (1H, s), 7.24-7.26 (3H, m), 7.43-7.45 (2H, m), 7.56 (2H, d), 7.64 (1H, s), 7.99 (2H, d), 10.39 (1H, s)

LCMS Spectrum: m/z (ESI+)(M+H)+=620; HPLC tR=3.26 min.

### 4-[4-[(3S)-3-Methylmorpholin-4-yl]-6-[1-[(4-methyl-1 3-thiazol-2-yl)sulfonyl]cyclopentyl]pyrimidin-2-yl]aniline

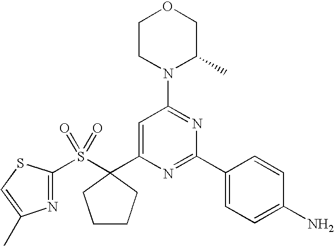

Bis(triphenylphosphine)palladium (II) chloride (0.162 g, 0.23 mmol) was added to 2-chloro-4-[(3S)-3-methylmorpholin-4-yl]-6-[1-[(4-methyl-1,3-thiazol-2-yl)sulfonyl]cyclopentyl]pyrimidine (2.05 g, 4.63 mmol), 4-(4,4,5,5-tetramethyl-1,3,2-dioxaborolan-2-yl)aniline (1.318 g, 6.02 mmol) and 2M aqueous sodium carbonate (8.33 mL, 16.66 mmol) in DME (10 mL), ethanol (10 mL) and water (25 mL) and the resulting mixture stirred at 80° C. for 18 hours. The cooled reaction mixture was diluted with ethyl acetate (100 mL), and washed sequentially with water (100 mL) and saturated brine (100 mL). The organic layer was dried (MgSO₄), filtered and evaporated. The crude product was purified by flash silica chromatography, elution gradient 0 to 75% ethyl acetate in DCM, to give the desired material as a yellow gum (0.84 g).

NMR Spectrum: ¹H NMR (400.132 MHz, DMSO-d₆) δ 1.19 (3H, d), 1.57-1.62 (2H, m), 1.83-1.89 (2H, m), 2.40 (3H, s), 2.73-2.78 (4H, m), 3.08-3.15 (1H, m), 3.44-3.50 (1H, m), 3.61-3.64 (1H, m), 3.75 (1H, d), 3.94-3.97 (1H, m), 4.10 (1H, d), 4.43-4.49 (1H, m), 5.50 (2H, s), 6.53 (2H, d), 6.54 (1H, s), 7.64 (1H, s), 7.73 (2H, d)

LCMS Spectrum: m/z (ESI+)(M+H)+=500; HPLC tR=2.66 min.

### 2-Chloro-4-[(3S)-3-methylmorpholin-4-yl]-6-[1-[(4-methyl-1,3-thiazol-2-yl)sulfonyl]cyclopentyl]pyrimidine

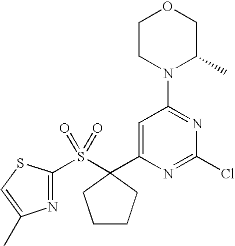

1,4-Dibromobutane (0.627 mL, 5.30 mmol) was added to 2-chloro-4-[(3S)-3-methylmorpholin-4-yl]-6-[(4-methyl-1,3-thiazol-2-yl)sulfonylmethyl]pyrimidine (2.06 g, 5.30 mmol), 40% aqueous sodium hydroxide solution (5.30 mL, 53 mmol) and tetrabutylammonium bromide (0.342 g, 1.06 mmol) in toluene (100 mL) and the resulting solution stirred at 60° C. for 3 hours. The reaction mixture was concentrated and diluted with ethyl acetate (100 mL), and washed sequentially with water (100 mL) and saturated brine (100 mL). The organic layer was dried (MgSO₄), filtered and evaporated to give the desired material as an orange gum (2.55 g).

NMR Spectrum: ¹H NMR (400.132 MHz, DMSO-d₆) δ δ 1.18 (3H, d), 1.57-1.62 (2H, m), 1.78-1.86 (2H, m), 2.45 (3H, s), 2.55-2.68 (4H, m), 3.10-3.17 (1H, m), 3.39-3.46 (1H, m), 3.55-3.59 (1H, m), 3.71 (1H, d), 3.90-3.99 (2H, m), 4.32-4.38 (1H, m), 6.72 (1H, s), 7.87 (1H, s)

LCMS Spectrum: m/z (ESI+)(M+H)+=443; HPLC tR=2.66 min.

The preparation of 2-chloro-4-[(3S)-3-methylmorpholin-4-yl]-6-[(4-methyl-1,3-thiazol-2-yl)sulfonylmethyl]pyrimidine was described earlier.

### EXAMPLE 37

The following compounds were prepared according to the following general procedure. The appropriate aniline was treated with 1,1′-thiocarbonyldiimidazole in a solvent mixture of DCM and THF at RT for 2-16 hours. Triethylamine and the appropriate amine were added and the reactions stirred at RT for an additional 1-16 hours (unless otherwise stated). The mixtures were purified by prep HPLC.

| | | | | Reten- |
|---|---|---|---|---|
| | | | | tion |
| | | | LCMS | time |
| Example | Structure | NAME | MH+ | (min) |
| 37a | 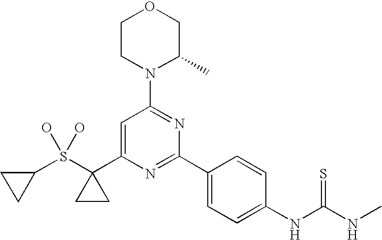 | 1-[4-[4-(1-cyclopropylsulfonylcyclopropyl)-6-[(3S)-3-methylmorpholin-4-yl]pyrimidin-2-yl]phenyl]-3-methylthiourea | 488 | 2.14 |
| 37b | 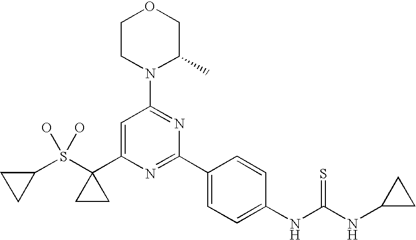 | 3-cyclopropyl-1-[4-[4-(1-cyclopropylsulfonylcyclopropyl)-6-[(3S)-3-methylmorpholin-4-yl]pyrimidin-2-yl]phenyl]thiourea | 514 | 2.28 |
| 37c | 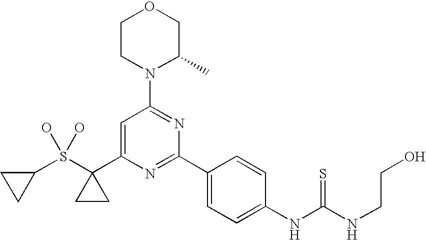 | 1-[4-[4-(1-cyclopropylsulfonylcyclopropyl)-6-[(3S)-3-methylmorpholin-4-yl]pyrimidin-2-yl]phenyl]-3-(2-hydroxyethyl)thiourea | 518 | 1.95 |
| 37d | 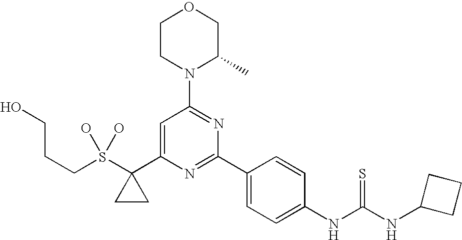 | 3-cyclobutyl-1-[4-[4-[1-(3-hydroxypropylsulfonyl)cyclopro-pyl]-6-[(3S)-3-methylmorpholin-4-yl]pyrimidin-2-yl]phenyl]thiourea | 546 | 2.32 |
| 37e | 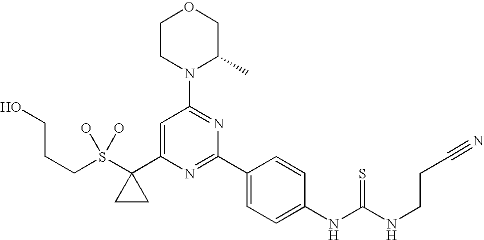 | 3-(2-cyanoethyl)-1-[4-[4-[1-(3-hydroxypropylsulfonyl)cyclopro-pyl]-6-[(3S)-3-methylmorpholin-4-yl]pyrimidin-2-yl]phenyl]thiourea | 545 | 2.0 |
| 37f | 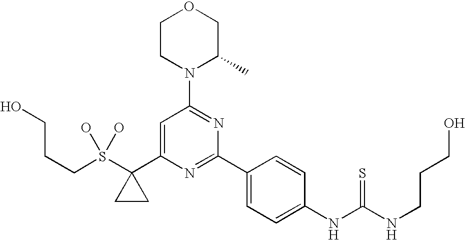 | 3-(3-hydroxypropyl)-1-[4-[4-[1-(3-hydroxypropylsulfonyl)cyclopro-pyl]-6-[(3S)-3-methylmorpholin-4-yl]pyrimidin-2-yl]phenyl]thiourea | 572 | 1.78 |
| 37g | 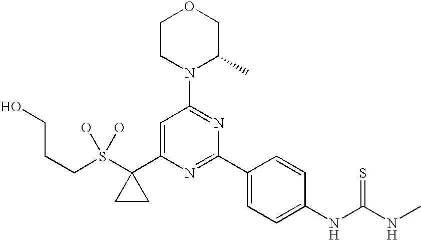 | 1-[4-[4-[1-(3-hydroxypropylsulfonyl)cyclopro-pyl]-6-[(3S)-3-methylmorpholin-4-yl]pyrimidin-2-yl]phenyl]-3-methylthiourea | 506 | 1.75 |
| 37h | 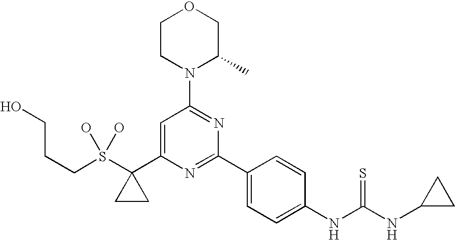 | 3-cyclopropyl-1-[4-[4-[1-(3-hydroxypropylsulfonyl)cyclopro-pyl]-6-[(3S)-3-methylmorpholin-4-yl]pyrimidin-2-yl]phenyl]thiourea | 532 | 1.87 |
| 37i | 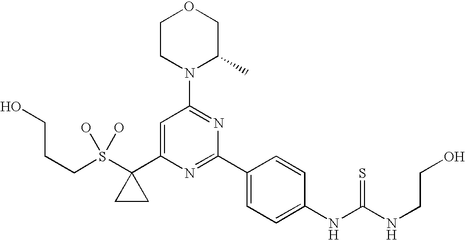 | 3-(2-hydroxyethyl)-1-[4-[4-[1-(3-hydroxypropylsulfonyl)cyclopro-pyl]-6-[(3S)-3-methylmorpholin-4-yl]pyrimidin-2-yl]phenyl]thiourea | 536 | 1.60 |
| 37j | 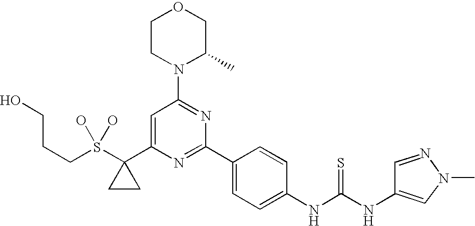 | 1-[4-[4-[1-(3-hydroxypropylsulfonyl)cyclopro-pyl]-6-[(3S)-3-methylmorpholin-4-yl]pyrimidin-2-yl]phenyl]-3-(1-methylpyrazol-4-yl)thiourea | 572 | 1.72 |
| 37k | 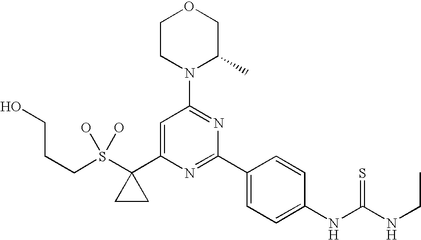 | 1-ethyl-3-[4-[4-[1-(3-hydroxypropylsulfonyl)cyclopro-pyl]-6-[(3S)-3-methylmorpholin-4-yl]pyrimidin-2-yl]phenyl]thiourea | 520 | 1.91 |
| 37l | 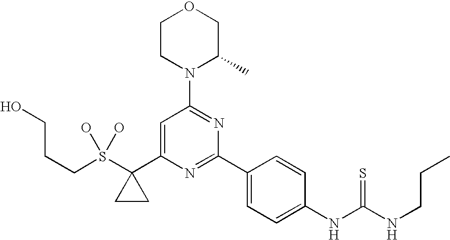 | 3-[4-[4-[1-(3-hydroxypropylsulfonyl)cyclopro-pyl]-6-[(3S)-3-methylmorpholin-4-yl]pyrimidin-2-yl]phenyl]-1-propylthiourea | 534 | 2.07 |
| 37m | 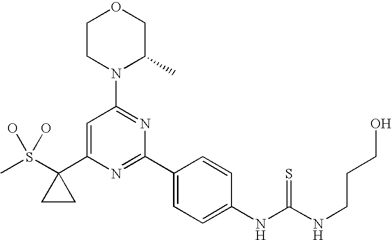 | 3-(3-hydroxypropyl)-1-[4-[4-[(3S)-3-methylmorpholin-4-yl]-6-(1-methylsulfonylcyclopropyl)pyri-midin-2-yl]phenyl]thiourea | 506.5 | 1.74 |
| 37n | 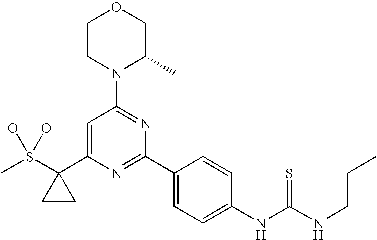 | 1-[4-[4-[(3S)-3-methylmorpholin-4-yl]-6-(1-methylsulfonylcyclopropyl)pyri-midin-2-yl]phenyl]-3-propylthiourea | 490.5 | 2.24 |
| 37o | 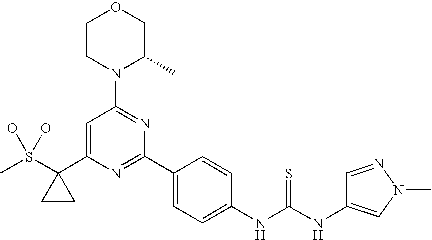 | 1-[4-[4-[(3S)-3-methylmorpholin-4-yl]-6-(1-methylsulfonylcyclopropyl)pyri-midin-2-yl]phenyl]-3-(1-methylpyrazol-4-yl)thiourea | 528.5 | 1.84 |
| 37p | 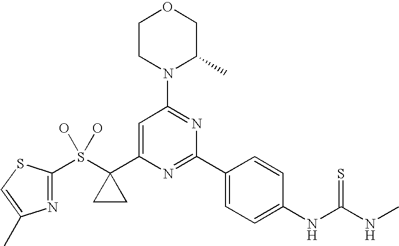 | 3-methyl-1-[4-[4-[(3S)-3-methylmorpholin-4-yl]-6-[1-[(4-methyl-1,3-thiazol-2-yl)sulfonyl]cyclopropyl]pyri-midin-2-yl]phenyl]thiourea | 545 | 2.38 |
| 37q | 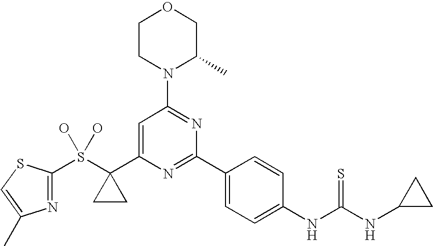 | 3-cyclopropyl-1-[4-[4-[(3S)-3-methylmorpholin-4-yl]-6-[1-[(4-methyl-1,3-thiazol-2-yl)sulfonyl]cyclopropyl]pyri-midin-2-yl]phenyl]thiourea | 571 | 2.51 |
| 37r | 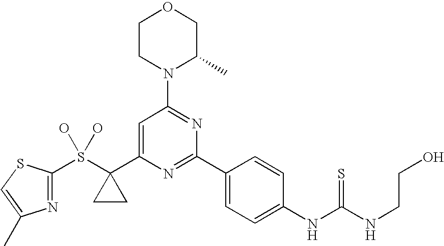 | 3-(2-hydroxyethyl)-1-[4-[4-[(3S)-3-methylmorpholin-4-yl]-6-[1-[(4-methyl-1,3-thiazol-2-yl)sulfonyl]cyclopropyl]pyri-midin-2-yl]phenyl]thiourea | 575 | 2.13 |
| 37s | 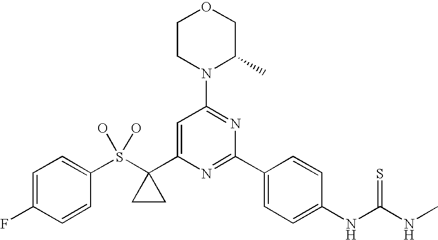 | 1-[4-[4-[1-(4-fluorophenyl)sulfonylcyclopropyl]-6-[(3S)-3-methylmorpholin-4-yl]pyrimidin-2-yl]phenyl]-3-methylthiourea | 540 | 2.52 |
| 37t | 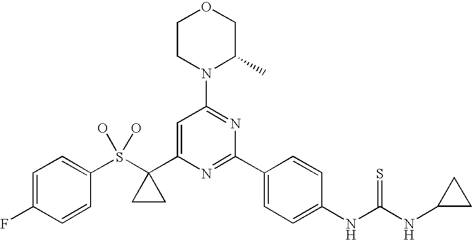 | 3-cyclopropyl-1-[4-[4-[1-(4-fluorophenyl)sulfonylcyclopropyl]-6-[(3S)-3-methylmorpholin-4-yl]pyrimidin-2-yl]phenyl]thiourea | 568 | 2.65 |
| 37u | 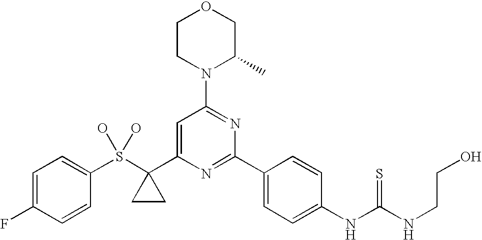 | 1-[4-[4-[1-(4-fluorophenyl)sulfonylcyclopropyl]-6-[(3S)-3-methylmorpholin-4-yl]pyrimidin-2-yl]phenyl]-3-(2-hydroxyethyl)thiourea | 572 | 2.27 |
| 37v | 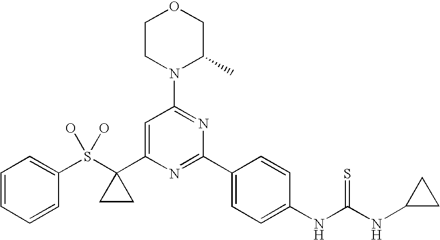 | 1-[4-[4-[1-(benzenesulfonyl)cyclopropyl]-6-[(3S)-3-methylmorpholin-4-yl]pyrimidin-2-yl]phenyl]-3-cyclopropylthiourea | 550 | 2.59 |
| 37w | 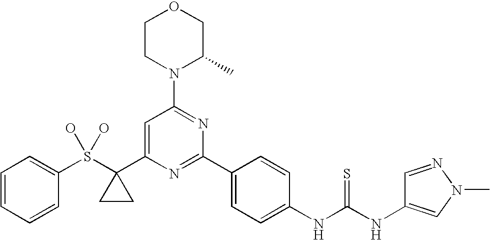 | 1-[4-[4-[1-(benzenesulfonyl)cyclopropyl]-6-[(3S)-3-methylmorpholin-4-yl]pyrimidin-2-yl]phenyl]-3-(1-methylpyrazol-4-yl)thiourea | 589 | 2.30 |
| 37x | 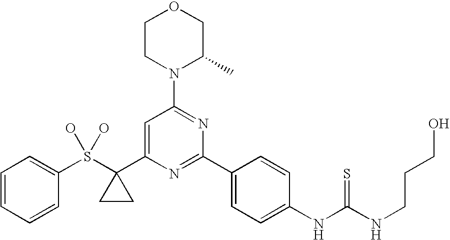 | 1-[4-[4-[1-(benzenesulfonyl)cyclopropyl]-6-[(3S)-3-methylmorpholin-4-yl]pyrimidin-2-yl]phenyl]-3-(3-hydroxypropyl)thiourea | 568 | 2.20 |
| 37y | 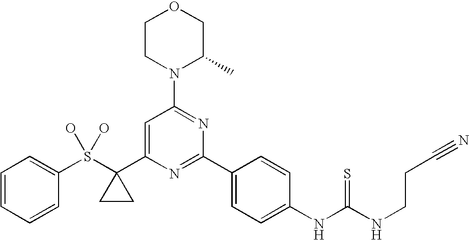 | 1-[4-[4-[1-(benzenesulfonyl)cyclopropyl]-6-[(3S)-3-methylmorpholin-4-yl]pyrimidin-2-yl]phenyl]-3-(2-cyanoethyl)thiourea | 563 | 2.49 |
| 37z | 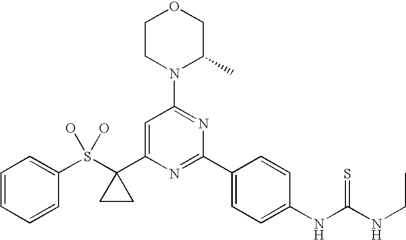 | 3-[4-[4-[1-(benzenesulfonyl)cyclopropyl]-6-[(3S)-3-methylmorpholin-4-yl]pyrimidin-2-yl]phenyl]-1-ethylthiourea | 538 | 2.60 |
| 37aa | 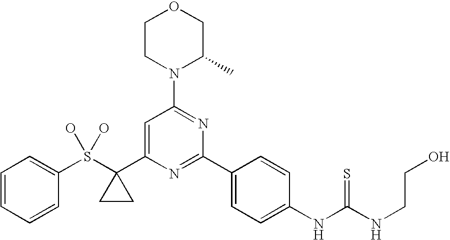 | 1-[4-[4-[1-(benzenesulfonyl)cyclopropyl]-6-[(3S)-3-methylmorpholin-4-yl]pyrimidin-2-yl]phenyl]-3-(2-hydroxyethyl)thiourea | 554 | 2.18 |
| 37ab | 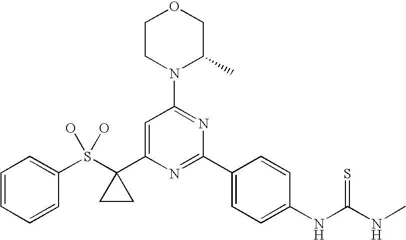 | 1-[4-[4-[1-(benzenesulfonyl)cyclopropyl]-6-[(3S)-3-methylmorpholin-4-yl]pyrimidin-2-yl]phenyl]-3-methylthiourea | 524 | 2.41 |
| 37ac | 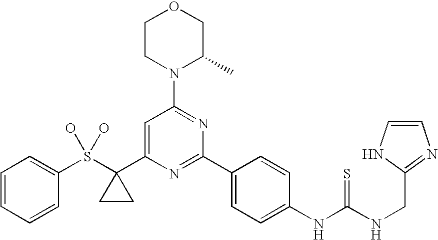 | 1-[4-[4-[1-(benzenesulfonyl)cyclopropyl]-6-[(3S)-3-methylmorpholin-4-yl]pyrimidin-2-yl]phenyl]-3-(1H-imidazol-2-ylmethyl)thiourea | 590 | 2.40 |
| 37ad* | 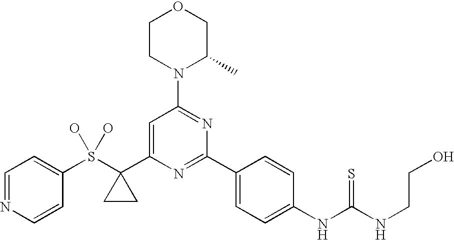 | 3-(2-hydroxyethyl)-1-[4-[4-[(3S)-3-methylmorpholin-4-yl]-6-(1-pyridin-4-ylsulfonylcyclopropyl)pyrimidin-2-yl]phenyl]thiourea | 556 | 1.92 |
| 37ae* | 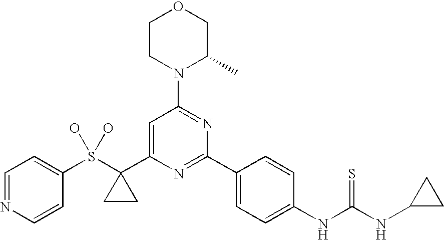 | 3-cyclopropyl-1-[4-[4-[(3S)-3-methylmorpholin-4-yl]-6-(1-pyridin-4-ylsulfonylcyclopropyl)pyrimidin-2-yl]phenyl]thiourea | 552 | 2.22 |
| 37af* | 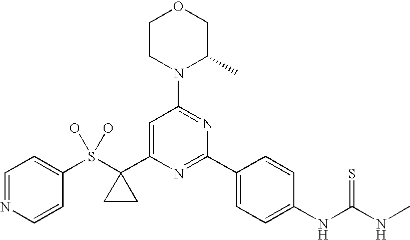 | 3-methyl-1-[4-[4-[(3S)-3-methylmorpholin-4-yl]-6-(1-pyridin-4-ylsulfonylcyclopropyl)pyrimidin-2-yl]phenyl]thiourea | 526 | 2.10 |
| 37ag | 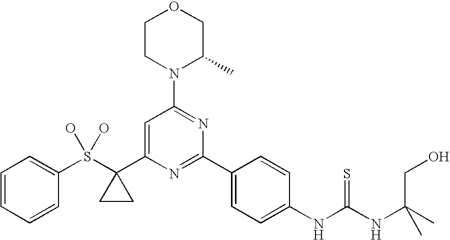 | 1-[4-[4-[1-(benzenesulfonyl)cyclopropyl]-6-[(3S)-3-methylmorpholin-4-yl]pyrimidin-2-yl]phenyl]-3-(1-hydroxy-2-methylpropan-2-yl)thiourea | 582 | 2.50 |

*The appropriate aniline was treated with 1,1′-thiocarbonyldiimidazole in a solvent mixture of DCM and THF at RT for 16 hours. Triethylamine and the appropriate amine, dissolved in DMF, were added and the reactions stirred at 50° C. for 1 hour. The mixture was purified by prep HPLC.

### EXAMPLE 37a

¹H NMR (400.132 MHz, DMSO-d₆) δ 0.90-0.98 (2H, m), 0.99-1.08 (2H, m), 1.24 (3H, d), 1.55-1.62 (2H, m), 1.63-1.70 (2H, m), 2.91-3.05 (4H, m), 3.19-3.25 (1H, m), 3.44-3.55 (1H, m), 3.64 (1H, d), 3.77 (1H, d), 3.98 (1H, d), 4.20 (1H, d), 4.56 (1H, s), 6.88 (1H, s), 7.55 (2H, d), 7.83 (1H, s), 8.27 (2H, d), 9.72 (1H, s).

mTOR Kinase Assay (Echo): 0.026 μM

### EXAMPLE 37b

¹H NMR (400.132 MHz, DMSO-d₆) δ 0.57-0.63 (2H, m), 0.72-0.78 (2H, m), 0.90-0.96 (2H, m), 0.99-1.07 (2H, m), 1.24 (3H, d), 1.55-1.62 (2H, m), 1.63-1.68 (2H, m), 2.90-3.04 (2H, m), 3.17-3.27 (1H, m), 3.50 (1H, d), 3.65 (1H, d), 3.77 (1H, d), 3.98 (1H, d), 4.18 (1H, d), 4.56 (1H, s), 6.89 (1H, s), 7.62 (2H, d), 8.27 (2H, d), 9.50 (1H, s).

mTOR Kinase Assay (Echo): 0.0124 μM

### EXAMPLE 37c

¹H NMR (400.132 MHz, DMSO-d₆) δ 0.90-0.96 (2H, m), 1.00-1.07 (2H, m), 1.24 (3H, d), 1.55-1.61 (2H, m), 1.64-1.69 (2H, m), 2.95-3.05 (1H, m), 3.17-3.27 (3H, m), 3.44-3.56 (3H, m), 3.64 (1H, d), 3.77 (1H, d), 3.98 (1H, d), 4.20 (1H, d), 4.55 (1H, s), 4.82 (1H, s), 6.88 (1H, s), 7.57-7.68 (2H, m), 7.86 (1H, s), 8.27 (2H, d), 9.81 (1H, s).

mTOR Kinase Assay (Echo): 0.00344 μM

### EXAMPLE 37d

¹H NMR (400.132 MHz, DMSO-d₆) δ 1.24 (3H, d), 1.54-1.60 (2H, m), 1.62-1.71 (3H, m), 1.89-1.98 (2H, m), 2.25-2.34 (2H, m), 2.42-2.52 (2H, m), 3.14-3.24 (3H, m), 3.41-3.55 (3H, m), 3.64 (1H, d), 3.77 (1H, d), 3.97 (1H, d), 4.20 (1H, d), 4.53-4.68 (2H, m), 6.81 (1H, s), 7.60 (2H, d), 8.11 (1H, s), 8.26 (2H, d), 9.58 (1H, s).

mTOR Kinase Assay (Echo): 0.00439 μM

### EXAMPLE 37e

¹H NMR (400.132 MHz, DMSO-d₆) δ 1.24 (3H, d), 1.55-1.60 (2H, m), 1.62-1.68 (2H, m), 1.91-1.98 (2H, m), 2.84-2.92 (2H, m), 3.13-3.24 (1H, m), 3.43-3.57 (5H, m), 3.64 (1H, d), 3.72-3.80 (3H, m), 3.97 (1H, d), 4.20 (1H, d), 4.57 (1H, s), 4.68 (1H, t), 6.83 (1H, s), 7.56 (2H, d), 8.16 (1H, s), 8.29 (2H, d), 9.98 (1H, s).

mTOR Kinase Assay (Echo): 0.0049 μM

### EXAMPLE 37f

¹H NMR (400.132 MHz, DMSO-d₆) δ 1.24 (3H, d), 1.55-1.59 (2H, m), 1.62-1.67 (2H, m), 1.93-1.98 (4H, m), 2.28-2.36 (2H, m), 2.65-2.70 (2H, m), 3.13-3.23 (1H, m), 3.44-3.56 (5H, m), 3.64 (1H, d), 3.77 (1H, d), 3.97 (1H, d), 4.20 (1H, d), 4.58 (1H, s), 4.71 (2H, s), 6.82 (1H, s), 7.00 (2H, s), 7.69 (2H, d), 8.21 (OH, s), 8.29 (2H, s).

mTOR Kinase Assay (Echo): 0.12 μM

### EXAMPLE 37g

¹H NMR (400.132 MHz, DMSO-d₆) δ 1.24 (3H, d), 1.54-1.59 (2H, m), 1.62-1.68 (2H, m), 1.89-1.99 (2H, m), 2.96 (3H, s), 3.14-3.28 (3H, m), 3.45-3.55 (3H, m), 3.64 (1H, d), 3.77 (1H, d), 3.97 (1H, d), 4.21 (1H, d), 4.58 (1H, s), 4.68 (1H, t), 6.82 (1H, s), 7.55 (2H, d), 7.84 (1H, s), 8.27 (2H, d), 9.73 (1H, s).

mTOR Kinase Assay (Echo): 0.0179 μM

### EXAMPLE 37h

¹H NMR (400.132 MHz, DMSO-d₆) δ 0.56-0.66 (2H, m), 0.72-0.80 (2H, m), 1.24 (3H, d), 1.53-1.60 (2H, 1m), 1.62-1.68 (2H, m), 1.88-2.00 (2H, m), 2.92 (1H, s), 3.16-3.27 (1H, m), 3.44-3.58 (5H, m), 3.64 (1H, d), 3.77 (1H, d), 3.98 (1H, d), 4.22 (1H, d), 4.57 (1H, s), 4.68 (1H, t), 6.82 (1H, s), 7.62 (2H, d), 8.14 (1H, s), 8.26 (2H, d), 9.50 (1H, s).

mTOR Kinase Assay (Echo): 0.0132 μM

### EXAMPLE 37i

¹H NMR (400.132 MHz, DMSO-d₆) δ 1.24 (3H, d), 1.54-1.59 (2H, m), 1.63-1.68 (2H, m), 1.89-1.97 (2H, m), 3.14-3.29 (5H, m), 3.46-3.60 (5H, m), 3.64 (1H, d), 3.77 (1H, d), 3.97 (1H, d), 4.21 (1H, d), 4.57 (1H, s), 4.68 (1H, t), 4.81 (1H, s), 6.82 (1H, s), 7.63 (2H, d), 7.96 (1H, s), 8.27 (2H, d), 9.81 (1H, s).

mTOR Kinase Assay (Echo): 0.00306 μM

### EXAMPLE 37j

¹H NMR (400.132 MHz, DMSO-d₆) δ 1.24 (3H, d), 1.54-1.60 (2H, m), 1.62-1.68 (2H, m), 1.89-1.99 (2H, m), 3.15-3.25 (1H, m), 3.41-3.56 (5H, m), 3.64 (1H, d), 3.82 (1H, d), 3.98 (1H, d), 4.22 (1H, d), 4.58 (1H, s), 4.69 (1H, t), 6.82 (1H, s), 7.51 (1H, s), 7.62 (2H, d), 8.05 (1H, s), 8.28 (2H, d), 9.69 (1H, s).

mTOR Kinase Assay (Echo): 0.0309 μM

### EXAMPLE 37k

¹H NMR (400.132 MHz, DMSO-d₆) δ 1.14 (3H, t), 1.24 (3H, d), 1.53-1.60 (2H, m), 1.62-1.67 (2H, m), 1.89-1.97 (2H, m), 3.14-3.28 (3H, m), 3.46-3.56 (5H, m), 3.64 (1H, d), 3.77 (1H, d), 3.97 (1H, d), 4.21 (1H, d), 4.58 (1H, s), 4.68 (1H, t), 6.82 (1H, s), 7.56 (2H, d), 7.88 (1H, s), 8.27 (2H, d), 9.62 (1H, s).

mTOR Kinase Assay (Echo): 0.0114 μM

### EXAMPLE 37l

¹H NMR (400.132 MHz, DMSO-d₆) δ 0.91 (3H, t), 1.24 (3H, d), 1.53-1.60 (4H, m), 1.62-1.67 (2H, m), 1.90-1.97 (2H, m), 3.16-3.27 (3H, m), 3.42-3.53 (5H, m), 3.64 (1H, d), 3.77 (1H, d), 3.97 (1H, d), 4.21 (1H, d), 4.57 (1H, s), 4.68 (1H, t), 6.82 (1H, s), 7.58 (2H, d), 7.89 (1H, s), 8.26 (2H, d), 9.63 (1H, s).

mTOR Kinase Assay (Echo): 0.032 μM

### EXAMPLE 37m

¹H NMR (400.132 MHz, DMSO-d₆) δ 1.24 (3H, d), 1.55-1.58 (2H, m), 1.67-1.75 (4H, m), 3.17-3.26 (1H, m), 3.28 (3H, s), 3.45-3.58 (5H, m), 3.64 (1H, dd), 3.77 (1H, d), 3.97 (1H, dd), 4.18-4.25 (1H, m), 4.52-4.63 (2H, m), 6.80 (1H, s), 7.57-7.60 (2H, m), 7.91 (1H, s), 8.24-8.27 (2H, m), 9.71 (1H, s).

mTOR Kinase Assay (Echo): 0.0506 μM

### EXAMPLE 37n

¹H NMR (400.132 MHz, DMSO-d₆) δ 0.91 (3H, t), 1.24 (3H, d), 1.53-1.62 (4H, m), 1.66-1.69 (2H, m), 3.22 (1H, td), 3.27 (3H, s), 3.43-3.52 (3H, m), 3.64 (1H, dd), 3.77 (1H, d), 3.97 (1H, dd), 4.18-4.25 (1H, m), 4.56-4.62 (1H, m), 6.80 (1H, s), 7.60 (2H, d), 7.91 (1H, s), 8.24-8.28 (2H, m), 9.65 (1H, s).

mTOR Kinase Assay (Echo): 0.0393 μM

### EXAMPLE 37o

¹H NMR (400.132 MHz, DMSO-d₆) δ 1.24 (3H, d), 1.56-1.59 (2H, m), 1.67-1.70 (2H, m), 3.22 (1H, td), 3.27 (3H, s), 3.49 (1H, td), 3.64 (1H, dd), 3.77 (1H, d), 3.81 (3H, s), 3.98 (1H, dd), 4.19-4.25 (1H, m), 4.57-4.63 (1H, m), 6.81 (1H, s), 7.51 (1H, s), 7.62-7.65 (2H, m), 8.05 (1H, s), 8.25-8.29 (2H, m), 9.67-9.76 (2H, m).

mTOR Kinase Assay (Echo): 0.033 μM

### EXAMPLE 37p

¹H NMR (400.132 MHz, DMSO-d₆) δ 1.22 (3H, d), 1.78-1.81 (2H, m), 1.95-1.98 (2H, m), 2.48 (3H, s), 2.95 (3H, d), 3.16-3.23 (1H, m), 3.44-3.51 (1H, m), 3.62 (1H, d), 3.76 (1H, d), 3.96-3.99 (1H, m), 4.14-4.18 (1H, m), 4.44-4.50 (1H, m), 6.82 (1H, s), 7.47 (2H, d), 7.80 (1H, s), 7.84 (1H, s), 7.96 (2H, d), 9.70 (1H, s).

mTOR Kinase Assay (Echo): 0.00534 μM

### EXAMPLE 37q

¹H NMR (400.132 MHz, DMSO-d₆) δ 0.58-0.62 (2H, m), 0.74-0.79 (2H, m), 1.22 (3H, d), 1.78-1.81 (2H, m), 1.95-1.98 (2H, m), 2.49 (3H, s), 2.87-2.97 (1H, m), 3.17-3.23 (1H, m), 3.44-3.51 (1H, m), 3.61-3.64 (1H, m), 3.76 (1H, d), 3.96-3.99 (1H, m), 4.15-4.18 (1H, m), 4.44-4.51 (1H, m), 6.83 (1H, s), 7.54 (2H, d), 7.84 (1H, s), 7.95 (2H, d), 8.15 (1H, s), 9.47 (1H, s).

mTOR Kinase Assay (Echo): 0.00294 μM

### EXAMPLE 37r

¹H NMR (400.132 MHz, DMSO-d₆) δ 1.22 (3H, d), 1.78-1.81 (2H, m), 1.94-1.98 (2H, m), 2.48 (3H, s), 3.16-3.23 (1H, m), 3.44-3.50 (1H, m), 3.57 (4H, s), 3.61-3.64 (1H, m), 3.76 (1H, d), 3.95-3.99 (1H, m), 4.15-4.18 (1H, m), 4.43-4.52 (1H, m), 4.82 (1H, s), 6.82 (1H, s), 7.54 (2H, d), 7.84 (2H, s), 7.95 (2H, d), 9.78 (1H, s).

mTOR Kinase Assay (Echo): 0.00162 μM

### EXAMPLE 37s

¹H NMR (400.132 MHz, DMSO-d₆) δ 1.19 (3H, d), 1.59-1.65 (2H, m), 1.89-1.91 (2H, m), 2.94 (3H, d), 3.16 (1H, dt), 3.47 (1H, dt), 3.61 (1H, dd), 3.75 (1H, d), 3.96 (1H, dd), 4.14 (1H, d), 4.44 (1H, s), 6.69 (1H, s), 7.44 (4H, t), 7.80-7.90 (5H, m), 9.70 (1H, s).

mTOR Kinase Assay (Echo): 0.00756 μM

### EXAMPLE 37t

¹H NMR (400.132 MHz, DMSO-d₆) δ 0.57-0.62 (2H, m), 0.74-0.79 (2H, m), 1.19 (3H, d), 1.61-1.63 (2H, m), 1.89-1.91 (2H, m), 2.90-2.94 (1H, m), 3.17 (1H, dt), 3.47 (1H, dt), 3.62 (1H, dd), 3.75 (1H, d), 3.96 (1H, dd), 4.14 (1H, d), 4.45 (1H, s), 6.69 (1H, s), 7.42 (2H, t), 7.52 (2H, d), 7.83-7.89 (4H, m), 8.13 (1H, s), 9.47 (1H, s).

mTOR Kinase Assay (Echo): 0.00889 μM

### EXAMPLE 37u

¹H NMR (400.132 MHz, DMSO-d₆) δ 1.19 (4H, d), 1.59-1.63 (3H, m), 1.89-1.91 (2H, m), 3.13-3.20 (1H, m), 3.43-3.50 (1H, m), 3.56 (4H, m), 3.60-3.63 (1H, m), 3.75 (1H, d), 3.96 (1H, dd), 4.14 (1H, d), 4.44 (1H, s), 4.81 (1H, s), 6.68 (1H, s), 7.42 (2H, t), 7.53 (2H, d), 7.83-7.86 (1H, m), 7.88 (2H, d), 9.78 (1H, s).

mTOR Kinase Assay (Echo): 0.000395 μM

### EXAMPLE 37v

¹H NMR (400.132 MHz, DMSO-d₆) δ 0.58-0.62 (2H, m), 0.74-0.79 (2H, m), 1.17-1.19 (3H, d), 1.62-1.69 (2H, m), 1.88-1.93 (2H, m), 2.92 (1H, bs), 3.12-3.19 (1H, td), 3.43-3.50 (1H, td), 3.60-3.63 (1H, dd), 3.73-3.76 (1H, d), 3.94-3.98 (1H, dd), 4.10-4.13 (1H, d), 4.41 (1H, bs), 6.67 (1H, s), 7.49-7.52 (2H, d), 7.58-7.62 (2H, t), 7.70-7.74 (1H, tt), 7.79-7.81 (2H, d), 7.89-7.91 (2H, d), 8.10 (1H, bs), 9.47 (1H, bs).

mTOR Kinase Assay (Echo): 0.00549 μM

### EXAMPLE 37w

¹H NMR (400.132 MHz, DMSO-d₆) δ 1.15-1.20 (3H, d), 1.60-1.70 (2H, m), 1.90-1.95 (2H, bs), 3.12-31.7 (1H, m), 3.25-3.30 (1H, m obscured by solvent peak), 3.45-3.50 (1H, m), 3.6 (1H, d), 3.7 (1H, d), 3.8 (3H, s), 4.0 (1H, m), 4.1 (1H, d), 4.40-4.45 (1H, bs), 6.7 (1H, s), 7.49-7.53 (3H, m), 7.68-7.73 (2H, m), 7.7 (1H, t), 7.8 (2H, d), 7.9 (2H, d), 8.0 (1H, bs), 9.7 (1H, bs).

mTOR Kinase Assay (Echo): 0.00137 μM

### EXAMPLE 37x

¹H NMR (400.132 MHz, DMSO-d₆) δ 1.17-1.19 (3H, d), 1.61-1.67 (2H, m), 1.68-1.75 (2H, m), 1.88-1.93 (2H, m), 3.11-3.17 (1H, td), 3.43-3.55 (5H, m), 3.59-3.63 (1H, dd), 3.73-3.76 (1H, d), 3.94-3.98 (1H, dd), 4.09-4.12 (1H, d), 4.40 (1H, bs), 4.53-4.54 (1H, t), 6.66 (1H, s), 7.45-7.47 (2H, d), 7.58-7.62 (2H, t), 7.70-7.75 (1H, tt), 7.79-7.81 (2H, dd), 7.86 (1H, bs), 7.89-7.92 (2H, d), 9.64 (1H, bs).

mTOR Kinase Assay (Echo): 0.00944 μM

### EXAMPLE 37y

¹H NMR (400.132 MHz, DMSO-d₆) δ 1.17-1.19 (3H, d), 1.61-1.68 (2H, m), 1.89-1.92 (2H, m), 2.85-2.88 (2H, t), 3.11-3.19 (1H, td), 3.43-3.49 (1H, td), 3.59-3.63 (1H, dd), 3.73-3.75 (3H, m), 3.94-3.98 (1H, dd), 4.09-4.12 (1H, d), 4.41 (1H, bs), 6.67 (1H, s), 7.42-7.45 (2H, d), 7.57-7.61 (2H, t), 7.70-7.74 (1H, tt), 7.78-7.81 (2H, dd), 7.90-7.93 (2H, d), 8.08-8.11 (1H, t), 9.91 (1H, s).

mTOR Kinase Assay (Echo): 0.00553 μM

### EXAMPLE 37z

¹H NMR (400.132 MHz, DMSO-d₆) δ 1.12-1.19 (6H, m), 1.61-1.69 (2H, m), 1.89-1.92 (2H, m), 3.11-3.19 (1H, td), 3.43-3.52 (3H, m), 3.59-3.63 (1H, dd), 3.73-3.76 (1H, d), 3.94-3.98 (1H, dd), 4.09-4.12 (1H, d), 4.40 (1H, bs), 6.66 (1H, s), 7.44-7.46 (2H, d), 7.58-7.62 (2H, t), 7.70-7.74 (1H, tt), 7.78-7.81 (2H, d), 7.84 (1H, bs), 7.90-7.92 (2H, d), 9.58 (1H, bs).

mTOR Kinase Assay (Echo): 0.00481 μM

### EXAMPLE 37aa

¹H NMR (400.132 MHz, DMSO-d₆) δ 1.17-1.19 (3H, d), 1.61-1.69 (2H, m), 1.88-1.92 (2H, m), 3.11-3.19 (1H, td), 3.43-3.49 (1H, td), 3.51-3.63 (5H, m), 3.72-3.76 (1H, d), 3.94-3.98 (1H, dd), 4.09-4.12 (1H, d), 4.40 (1H, bs), 4.82 (1H, bs), 6.66 (1H, s), 7.51-7.53 (2H, d), 7.58-7.61 (2H, t), 7.70-7.74 (1H, tt), 7.78-7.81 (2H, d), 7.89-7.91 (3H, m), 9.83 (1H, bs).

mTOR Kinase Assay (Echo): 0.00567 μM

### EXAMPLE 37ab

¹H NMR (400.132 MHz, DMSO-d₆) δ 1.17-1.19 (3H, d), 1.61-1.68 (2H, m), 1.88-1.92 (2H, m), 2.94-2.95 (3H, d), 3.11-3.17 (1H, td), 3.44-3.49 (1H, td), 3.58-3.63 (1H, dd), 3.73-3.76 (1H, d), 3.94-3.98 (1H, dd), 4.08-4.12 (1H, d), 4.41 (1H, bs), 6.67 (1H, s), 7.43-7.45 (2H, d), 7.58-7.62 (2H, t), 7.70-7.74 (1H, tt), 7.79-7.81 (3H, m), 7.90-7.92 (2H, d), 9.70 (1H, bs).

mTOR Kinase Assay (Echo): 0.003 μM

### EXAMPLE 37ac

¹H NMR (400.132 MHz, DMSO-d₆) δ 1.17-1.19 (3H, d), 1.62-1.69 (2H, m), 1.90-1.93 (2H, m), 3.12-3.18 (1H, td), 3.43-3.50 (1H, td), 3.59-3.63 (1H, dd), 3.73-3.76 (1H, d), 3.94-3.98 (1H, dd), 4.09-4.13 (1H, d), 4.41 (1H, bs), 4.71-4.72 (2H, d), 6.67 (1H, s), 7.00 (2H, bs), 7.56-7.62 (4H, m), 7.70-7.74 (1H, tt), 7.79-7.81 (2H, dd), 7.91-7.93 (2H, d), 8.17 (1H, bs), 9.98 (1H, bs), 11.98 (1H, bs).

mTOR Kinase Assay (Echo): 0.0298 μM

### EXAMPLE 37ad

¹H NMR (400.132 MHz, DMSO-d₆) δ 1.19 (3H, d), 1.70 (2H, q), 1.98 (2H, q), 3.10-3.20 (1H, td), 3.40-3.50 (1H, td), 3.50-3.58 (4H, m), 3.58 (1H, dd), 3.75 (1H, d), 3.95 (1H, dd), 4.15 (1H, d), 4.50 (1H, br s), 4.80 (1H, br s), 6.70 (1H, s), 7.50 (2H, d), 7.74-7.77 (4H, m), 7.82 (1H, s), 8.87 (2H, dd), 9.75 (1H, s)

### EXAMPLE 37ae

¹H NMR (400.132 MHz, DMSO-d₆) δ δ 0.60 (2H, q), 0.77 (2H, q), 1.20 (3H, d), 1.70 (2H, q), 1.98 (2H, q), 2.85-2.95 (1H, m), 3.19 (1H, td), 3.47 (1H, td), 3.61 (1H, dd), 3.75 (1H, d), 3.96 (1H, dd), 4.17 (1H, d), 4.48 (1H, br s), 6.71 (1H, s), 7.49 (2H, d), 7.72 (2H, d), 7.78 (2H, dd), 8.15 (1H, br s), 8.87 (2H, dd), 9.46 (1H, br s)

### EXAMPLE 37af

¹H NMR (400.132 MHz, DMSO-d₆) δ 1.19 (3H, d), 1.70 (2H, q), 1.98 (2H, q), 2.94 (3H, d), 3.17 (1H, td), 3.46 (1H, td), 3.61 (1H, dd), 3.75 (1H, d), 3.96 (1H, dd), 4.10-4.20 (1H, d), 4.50 (1H, br s), 6.71 (1H, s), 7.43 (2H, d), 7.74-7.76 (2H, d), 7.77-7.79 (2H, dd), 7.82 (1H, m), 8.87 (2H, dd), 9.70 (1H, br s).

### EXAMPLE 37ag

¹H NMR (399.902 MHz, DMSO-d₆) δ 1.17 (3H, d), 1.45 (6H, s), 1.65 (2H, m), 1.90 (2H, m), 3.14 (1H, m), 3.46 (1H, m), 3.58 (3H, m), 3.75 (1H, m), 3.96 (1H, m), 4.11 (1H, m), 4.41 (1H, m), 6.67 (1H, s), 7.39 (1H, m), 7.50 (2H, m), 7.60 (2H, m), 7.75 (3H, m), 7.88 (2H, m)

The preparations of the anilines required for Examples 37a -37ag have been described earlier.

### EXAMPLE 38

### 3-Cyclopropyl-1-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]urea

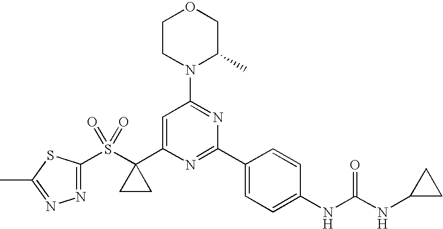

Cyclopropylamine (84 mg, 1.48 mmol) was added to phenyl N-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]carbamate (175 mg, 0.30 mmol) and triethylamine (0.205 mL, 1.48 mmol) in NMP (2 mL) at RT and the reaction was allowed to stir for 2 hours. The mixture was purified by preparative HPLC, using decreasingly polar mixtures of water (containing 1% ammonia) and acetonitrile as eluents, to give the desired material as a cream solid (112 mg).

NMR Spectrum: ¹H NMR (400.132 MHz, DMSO-d₆) δ 0.37-0.45 (2H, m), 0.61-0.68 (2H, m), 1.21 (3H, d), 1.78-1.88 (2H, m), 1.97-2.05 (2H, m), 2.83 (3H, s), 3.12-3.23 (1H, m), 3.42-3.52 (1H, m), 3.61 (1H, d), 3.75 (1H, d), 3.96 (1H, d), 4.20 (1H, s), 4.52 (1H, s), 6.46 (1H, t), 6.77 (1H, s), 7.42 (2H, d), 7.74 (2H, d), 8.57 (1H, s)

LCMS Spectrum: m/z (ESI+)(M+H)+=556; HPLC tR=2.25 min.

mTOR Kinase Assay (Echo): 0.00131 μM

The following compounds were made in an analogous fashion from phenyl N-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]carbamate and the appropriate amine.

| | | | LCMS | Retention |
|---|---|---|---|---|
| Example | Structure | NAME | MH+ | time (min) |
| 38a | 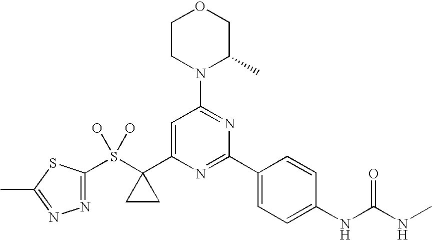 | 3-methyl-1-[4-[4-[(3S)-3-methylmorpholin-4-yl]-6-[1-[(5-methyl-1,3,4-thiadiazol-2-yl)sulfonyl]cyclopropyl]pyri-midin-2-yl]phenyl]urea | 530 | 209 |

### EXAMPLE 38a

¹H NMR (400.132 MHz, DMSO-d₆) δ 1.21 (3H, d), 1.82-1.85 (2H, m), 1.98-2.04 (2H, m), 2.66 (3H, s), 2.82 (3H, s), 3.13-3.25 (1H, m), 3.40-3.47 (1H, m), 3.61 (1H, d), 3.76 (1H, d), 3.96 (1H, d), 4.21 (1H, s), 6.08 (1H, t), 6.77 (1H, s), 7.42 (2H, d), 7.74 (2H, d), 8.78 (1H, s).

mTOR Kinase Assay (Echo): 0.000918 μM

The preparation of phenyl N-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]carbamate is described below.

### Phenyl N-[4-[4-[(3S)-3-methylmorpholin-4-yl]-6-[1-[(5-methyl-1,3,4-thiadiazol-2-yl)sulfonyl]cyclopropyl]pyrimidin-2-yl]phenyl]carbamate

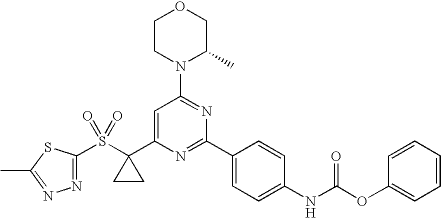

Phenyl chloroformate (0.398 mL, 3.17 mmol) was added slowly to 4-[4-[(3S)-3-methylmorpholin-4-yl]-6-[1-[(5-methyl-1,3,4-thiadiazol-2-yl)sulfonyl]cyclopropyl]pyrimidin-2-yl]aniline (1.25 g, 2.65 mmol) and sodium hydrogen carbonate (0.333 g, 3.97 mmol) in dioxane (30 mL) at 5° C. under an atmosphere of nitrogen. The resulting mixture was stirred at RT for 18 hours then the mixture diluted with ethyl actate (125 mL), and washed sequentially with water (2×100 mL). The organic layer was dried (Na₂SO₄), filtered and evaporated. The crude material was triturated with diethyl ether and isohexane to give a solid which was collected by filtration and dried under vacuum to give the desired material as a cream solid (1.24 g).

NMR Spectrum: ¹H NMR (400.132 MHz, DMSO-d₆) δ 1.22 (3H, d), 1.82-1.88 (2H, m), 2.00-2.05 (2H, m), 2.82 (3H, s), 3.20 (1H, dd), 3.42-3.50 (1H, m), 3.60 (1H, d), 3.76 (1H, d), 4.00 (1H, d), 4.23 (1H, s), 4.53 (1H, s), 6.81 (1H, s), 7.22-7.34 (3H, m), 7.40-7.50 (2H, m), 7.55 (2H, d), 7.84 (2H, d), 10.46 (1H, s)

LCMS Spectrum: m/z (ESI+)(M+H)+=593; HPLC tR=2.81 min.

### 4-[4-[(3S)-3-Methylmorpholin-4-yl]-6-[1-[(5-methyl-1,3,4-thiadiazol-2-yl)sulfonyl]cyclopropyl]pyrimidin-2-yl]aniline

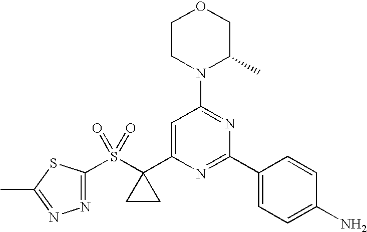

Bis(triphenylphosphine)palladium(II) chloride (0.164 g, 0.23 mmol) was added to 2-chloro-4-[(3S)-3-methylmorpholin-4-yl]-6-[1-[(5-methyl-1,3,4-thiadiazol-2-yl)sulfonyl]cyclopropyl]pyrimidine (1.45 g, 3.49 mmol), 4-(4,4,5,5-tetramethyl-1,3,2-dioxaborolan-2-yl)aniline (1.146 g, 5.23 mmol) and aqueous sodium carbonate solution (3 mL, 6.00 mmol) in a solvent mixture of DMF (10 mL), DME (2 mL), water (2 mL) and ethanol (2 mL). The atmosphere was replaced with nitrogen and the reaction stirred at 90° C. for 18 hours. The reaction mixture was diluted with ethyl acetate (200 mL), and washed with saturated brine (2×100 mL). The organic layer was dried (Na₂SO₄), filtered and evaporated to afford crude product. The crude product was purified by flash silica chromatography, elution gradient 10 to 80% ethyl acetate in isohexane, to give the desired material as a beige solid (1.25 g).

NMR Spectrum: ¹H NMR (400.132 MHz, DMSO-d₆) δ 1.19 (3H, d), 1.78-1.83 (2H, m), 1.98-2.01 (2H, m), 2.83 (3H, s), 3.10-3.21 (1H, m), 3.38-3.51 (1H, m), 3.60 (1H, d), 3.74 (1H, d), 3.95 (1H, d), 4.16 (1H, s), 4.48 (1H, s), 5.59 (2H, s), 6.51 (2H, d), 6.66 (1H, s), 7.57 (2H, d)

LCMS Spectrum: m/z (ESI+)(M+H)+=473; HPLC tR=2.14 min.

### 2-Chloro-4-[(3S)-3-methylmorpholin-4-yl]-6-[1-[(5-methyl-1,3,4-thiadiazol-2-yl)sulfonyl]cyclopropyl]pyrimidine

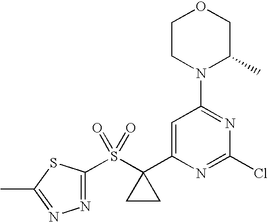

An aqueous solution of sodium hydroxide (20 mL, 532.5 mmol) was added to a stirred mixture of 2-chloro-4-[(3S)-3-methylmorpholin-4-yl]-6-[(5-methyl-1,3,4-thiadiazol-2-yl)sulfonylmethyl]pyrimidine (1.8 g, 4.62 mmol), 1,2-dibromoethane (1.99 ml, 23.08 mmol) and tetraethylammonium bromide (0.097 g, 0.46 mmol) in DCM (40 mL) at RT. The resulting mixture was stirred at RT for 24 hours then the mixture diluted with DCM (50 mL). The organic layer was dried (Na₂SO₄), filtered and evaporated to afford crude product which was chromatographed on silica, elution gradient 10 to 60% ethyl acetate in isohexane, to give the desired material as a yellow gum (1.48 g).

NMR Spectrum: ¹H NMR (400.132 MHz, DMSO-d₆) δ 1.18 (3H, d), 1.79-1.84 (2H, m), 1.94-2.01 (2H, m), 2.87 (3H, s), 3.17-3.23 (1H, m), 3.37-3.46 (1H, m), 3.55 (1H, dd), 3.71 (1H, d), 3.92 (1H, dd), 4.06 (1H, s), 4.33 (1H, s), 6.87 (1H, s) LCMS Spectrum: m/z (ESI+)(M+H)+=416; HPLC tR=1.98 min.

### 2-Chloro-4-[(3S)-3-methylmorpholin-4-yl]-6-[(5-methyl-1 3,4-thiadiazol-2-yl)sulfonylmethyl]pyrimidine

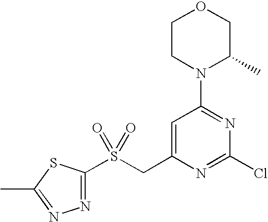

3-Chloroperoxybenzoic acid (4.77 g, 27.66 mmol) was added portionwise to 2-chloro-4-[(3S)-3-methylmorpholin-4-yl]-6-[(5-methyl-1,3,4-thiadiazol-2-yl)sulfanylmethyl]pyrimidine (3.3 g, 9.22 mmol), in DCM (70 mL) at RT under a nitrogen atmosphere. The resulting solution was stirred at RT for 2 hours then diluted with ethyl acetate (250 mL), and washed sequentially with a 10% aqueous solution of sodium metabisulphite (100 mL) and 2M sodium carbonate (100 mL). The organic layer was dried (Na₂SO₄), filtered and evaporated to afford crude product which was purified by flash silica chromatography, elution gradient 10 to 100% ethyl acetate in isohexane, to give the desired material as a white solid (2.22 g).

NMR Spectrum: ¹H NMR (400.132 MHz, DMSO-d₆) δ 1.19 (4H, d), 2.87 (4H, s), 3.15-3.26 (1H, m), 3.44 (1H, td), 3.59 (1H, d), 3.73 (1H, d), 3.94 (2H, m), 4.22 (1H, s), 5.03 (2H, s), 6.92 (1H, s)

LCMS Spectrum: m/z (ESI+)(M+H)+=390; HPLC tR=1.83 min.

### 2-Chloro-4-[(3S)-3-methylmorpholin-4-yl]-6-[(5-methyl-1 3 4-thiadiazol-2-yl)sulfanylmethyl]pyrimidine

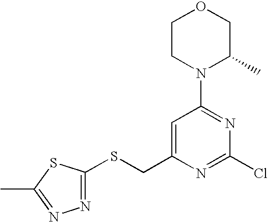

DIPEA (2.94 mL, 16.97 mmol) was added to 5-methyl-1,3,4-thiadiazole-2-thiol (1.645 g, 12.44 mmol), in acetonitrile (40 mL) at RT under an atmosphere of nitrogen. The resulting solution was stirred at RT for 20 minutes then 2-chloro-4-(iodomethyl)-6-[(3S)-3-methylmorpholin-4-yl]pyrimidine (4 g, 11.31 mmol) was added. The resulting mixture was stirred at RT for 1 hour then then mixture diluted with ethyl acetate (300 mL), and washed with water (150 mL).The organic layer was dried (MgSO₄), filtered and evaporated to afford crude product which was purified by flash silica chromatography, elution gradient 0 to 2% methanol in ethyl acetate, to give the desired material as a white solid (3.30 g).

NMR Spectrum: ¹H NMR (400.132 MHz, DMSO-d₆) δ 6.90 (1H, s), 1.17 (3H, d), 2.68 (3H, s), 3.12-3.22 (1H, m), 3.42 (1H, td), 3.57 (1H, dd), 3.71 (1H, d), 3.86-4.04 (2H, m), 4.27 (1H, s), 4.42 (2H, s)

The preparation of 2-chloro-4-(iodomethyl)-6-[(3S)-3-methylmorpholin-4-yl]pyrimidine was described earlier.

### EXAMPLE 39

### 3-Cyclopropyl-1-[4-[4-[(3S)-3-methylmorpholin-4-yl]-6-[1-(1,3-thiazol-2-ylsulfonyl)cyclopropyl]pyrimidin-2-yl]phenyl]urea

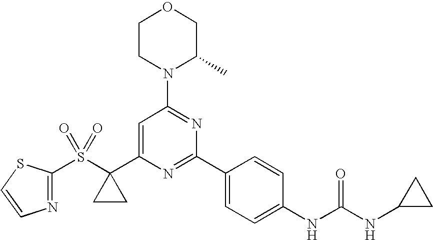

Bis(triphenylphosphine)palladium(II) chloride (17.6 mg, 0.03 mmol) was added to 2-chloro-4-[(3S)-3-methylmorpholin-4-yl]-6-[1-(1,3-thiazol-2-ylsulfonyl)cyclopropyl]pyrimidine (150 mg, 0.37 mmol), 1-cyclopropyl-3-(4-(4,4,5,5-tetramethyl-1,3,2-dioxaborolan-2-yl)phenyl)urea (170 mg, 0.56 mmol) and an aqueous solution of sodium carbonate (5 mL, 10.00 mmol) in a solvent mixture of DMF (2 mL), DME (16 mL), water (2 mL) and ethanol (2 mL). The atmosphere was replaced with nitrogen and the mixture stirred at 90° C. for 18 hours. The mixture was allowed to cool and diluted with ethyl acetate (200 mL) and washed with water (2×100 mL). The organic layer was dried (Na₂SO₄), filtered and evaporated to afford crude product which was purified by flash silica chromatography, elution gradient 0 to 4% methanol in ethyl acetate. The crude material was further purified by preparative HPLC, using decreasingly polar mixtures of water (containing 1% ammonia) and acetonitrile as eluents, to give the desired material (30 mg).

NMR Spectrum: ¹H NMR (400.132 MHz, DMSO-d₆) δ 0.37-0.45 (2H, m), 0.60-0.67 (2H, m), 1.19 (3H, d), 1.76-1.82 (2H, m), 1.95-2.02 (2H, m), 3.12-3.21 (1H, m), 3.45 (1H, d), 3.61 (1H, d), 3.75 (1H, d), 3.96 (1H, d), 4.18 (1H, s), 4.44 (1H, s), 6.45 (1H, s), 6.73 (1H, s), 7.41 (2H, d), 7.83 (2H, d), 8.24 (1H, s), 8.28 (1H, s), 8.54 (1H, s)

The following compound was made in an analogous fashion from 2-chloro-4-[(3S)-3-methylmorpholin-4-yl]0 -6-[1-(1,3-thiazol-2-ylsulfonyl)cyclopropyl]pyrimidine and the appropriate amine.

| | | | LCMS | Retention |
|---|---|---|---|---|
| Example | Structure | NAME | MH+ | time (min) |
| 39a | 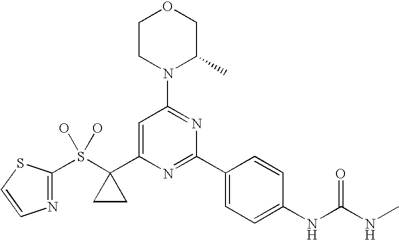 | 3-methyl-1-[4-[4-[(3S)-3-methylmorpholin-4-yl]-6-[1-(1,3-thiazol-2-ylsulfonyl)cyclopropyl]pyrimidin-2-yl]phenyl]urea | 515 | 2.05 |

### EXAMPLE 39a

¹H NMR (400.132 MHz, DMSO-d₆) δ 1.19 (3H, d), 1.76-1.81 (2H, m), 1.96-2.01 (2H, m), 2.66 (3H, d), 3.11-3.22 (1H, m), 3.42-3.49 (1H, m), 3.61 (1H, d), 3.75 (1H, d), 3.96 (1H, d), 4.17 (1H, s), 4.44 (1H, s), 6.07 (1H, q), 6.73 (1H, s), 7.40 (2H, d), 7.82 (2H, d), 8.24 (1H, d), 8.28 (1H, d), 8.75 (1H, s)

The preparation of 2-chloro-4-[(3S)-3-methylmorpholin-4-yl]-6-[1-(1,3-thiazol-2-ylsulfonyl)cyclopropyl]pyrimidine is described below.

2-Chloro-4-[(3S)-3-methylmorpholin-4-yl]-6-[1-(13-thiazol-2-ylsulfonyl)cyclopropyl]pyrimidine

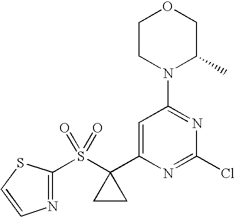

An aqueous solution of sodium hydroxide (0.235 mL, 6.27 mmol) was added to a stirred mixture of 2-chloro-4-[(3S)-3-methylmorpholin-4-yl]-6-(1,3-thiazol-2-ylsulfonylmethyl)pyrimidine (2.35 g, 6.27 mmol), 1,2-dibromoethane (2.70 mL, 31.34 mmol) and tetraethylammonium bromide (0.132 g, 0.63 mmol) in toluene at RT. The resulting mixture was stirred at 70° C. for 2 hours then was diluted with ethyl acetate (150 mL), and washed with water (100 mL). The organic layer was dried (Na₂SO₄), filtered and evaporated to afford crude product. The crude product was purified by flash silica chromatography, elution gradient 10 to 60% ethyl acetate in isohexane, to give the desired material as a yellow solid (2.45 g).

NMR Spectrum: ¹H NMR (400.132 MHz, DMSO-d₆) δ 1.18 (4H, d), 1.69-1.75 (2H, m), 1.91-1.96 (2H, m), 3.12-3.21 (1H, m), 3.40 (1H, d), 3.55 (1H, d), 3.70 (1H, d), 3.92 (1H, d), 4.00 (1H, s), 4.27 (1H, s), 6.84 (1H, s), 8.20 (1H, d), 8.33 (1H, d)

LCMS Spectrum: m/z (ESI+)(M+H)+=401; HPLC tR=2.04 min.

### 2-Chloro-4-[(3S)-3-methylmorpholin-4-yl]-6-(1,3-thiazol-2-ylsulfonylmethyl)pyrimidine

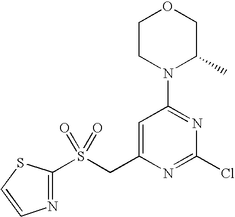

3-Chloroperoxybenzoic acid (6.04 g, 35.00 mmol) was added portionwise to 2-chloro-4-[(3S)-3-methylmorpholin-4-yl]-6-(1,3-thiazol-2-ylsulfanylmethyl)pyrimidine (4 g, 11.67 mmol), in DCM (10 mL) at RT under an atmosphere of nitrogen. The resulting solution was stirred at RT for 3 hours. The reaction mixture was diluted with ethyl acetate (250 mL), and washed with a 10% aqueous solution sodium metabisulphite (100 mL) and a saturated aqueous solution of sodium carbonate (100 mL). The organic layer was dried (Na₂SO₄), filtered and evaporated to afford crude product. The crude product was purified by flash silica chromatography, elution gradient 10 to 100% ethyl acetate in isohexane, to give the desired material as a white solid (2.85 g).

NMR Spectrum: ¹H NMR (400.132 MHz, DMSO-d₆) δ 8.33 (1H, d), 1.18 (4H, d), 3.14-3.25 (1H, m), 3.43 (1H, dd), 3.58 (1H, d), 3.72 (1H, d), 3.88-4.01 (2H, m), 4.20 (1H, s), 4.87 (2H, s), 6.82 (1H, s), 8.24 (1H, d)

LCMS Spectrum: m/z (ESI+)(M+H)+=375; HPLC tR=1.86 min.

### 2-Chloro-4-[(3S)-3-methylmorpholin-4-yl]-6-(1,3-thiazol-2-ylsulfanlmethyl)pyrimidine

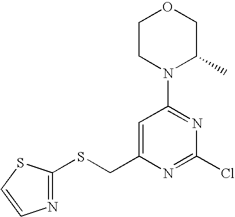

DIPEA (2.94 mL, 16.97 mmol) was added to 2-mercaptothiazole (1.458 g, 12.44 mmol), in acetonitrile (40 mL) at RT under an atmosphere of nitrogen. The resulting solution was stirred at RT for 20 minutes. 2-Chloro-4-(iodomethyl)-6-[(3S)-3-methylmorpholin-4-yl]pyrimidine (4 g, 11.31 mmol) was added and the mixture stirred for 1 hour. The reaction mixture was diluted with ethyl acetate (300 mL), and washed with water (100 mL). The organic layer was dried (MgSO₄), filtered and evaporated to afford crude product. The crude product was purified by flash silica chromatography, elution gradient 10 to 80% ethyl acetate in isohexane, to give the desired material as a colourless gum (3.77 g).

NMR Spectrum: ¹H NMR (400.132 MHz, DMSO-d₆) δ 1.15 (5H, d), 3.15 (1H, td), 3.41 (2H, td), 3.56 (1H, dd), 3.70 (1H, d), 3.91 (2H, m), 4.25 (1H, s), 4.36 (2H, s), 6.84 (1H, s), 7.70 (1H, d), 7.76 (1H, d)

LCMS Spectrum: m/z (ESI+)(M+H)+=343; HPLC tR=2.07 min.

The preparation of 2-chloro-4-(iodomethyl)-6-[(3S)-3-methylmorpholin-4-yl]pyrimidine was described earlier.

### EXAMPLE 40

### 1-[4-[4-[1-(1H-Imidazol-2-ylsulfonyl)cyclopropyl]-6-1(3S)-3-methylmorpholin-4-yl]pyrimidin-2-yl]phenyl]-3-methylurea

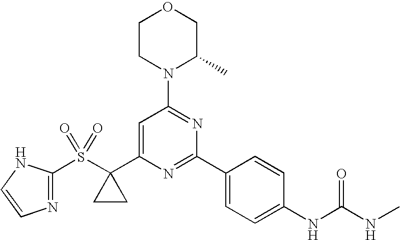

Anisole (0.159 mL, 1.46 mmol) was added to 1-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]-3-methylurea (180 mg, 0.29 mmol) in TFA (8 mL) at RT under an atmosphere of nitrogen. The resulting solution was stirred at 60° C. for 90 minutes then the solvent removed under reduced pressure and the residue chromatographed on an SCX column, eluting with 7M ammonia in methanol. The material was further purified by flash silica chromatography, elution gradient 10 to 90% ethyl acetate in isohexane, to give the desired material as a cream solid (122 mg).

NMR Spectrum: ¹H NMR (400.132 MHz, DMSO-d₆) δ 1.19 (3H, d), 1.68-1.74 (2H, m), 1.91-1.98 (2H, m), 2.66 (3H, s), 3.09-3.19 (1H, m), 3.44-3.50 (1H, m), 3.61 (1H, d), 3.76 (1H, d), 3.97 (1H, d), 4.13 (1H, s), 4.40 (1H, s), 6.05 (1H, s), 6.64 (1H, s), 7.35 (2H, s), 7.43 (2H, d), 8.00 (2H, d), 8.71 (1H, s)

LCMS Spectrum: m/z (ESI+)(M+H)+=493; HPLC tR=1.33 min.

mTOR Kinase Assay (Echo): 0.00436 μM

The following compound was made in an analogous fashion from 3-cyclopropyl-1-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]urea.

| | | | LCMS | Retention |
|---|---|---|---|---|
| Example | Structure | NAME | MH+ | time (min) |
| 40a | 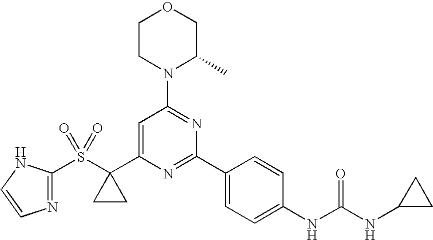 | 3-cyclopropyl-1-[4-[4-[1-(1H-imidazol-2-ylsulfonyl)cyclopropyl]-6-[(3S)-3-methylmorpholin-4-yl]pyrimidin-2-yl]phenyl]urea | 524 | 1.56 |

### EXAMPLE 40a

¹H NMR (400.132 MHz, DMSO-d₆) δ 0.38-0.44 (2H, m), 0.63-0.68 (2H, m), 1.19 (3H, d), 1.67-1.75 (2H, m), 1.89-1.99 (2H, m), 2.51-2.57 (1H, m), 3.11-3.22 (1H, m), 3.39-3.52 (1H, m), 3.61 (1H, d), 3.76 (1H, d), 3.97 (1H, d), 4.11 (1H, d), 4.39 (1H, s), 6.41 (1H, s), 6.65 (1H, s), 7.36 (2H, s), 7.44 (2H, d), 8.00 (2H, d), 8.50 (1H, s) 13.5(1H,s).

mTOR Kinase Assay (Echo): 0.00649 μM

The preparation of 1-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]-3-methylureais described below.

### 1-[4-[4-[1-[1-[(4-Methoxyphenyl)methyl]imidazol-2-yl]sulfonylcyclopropyl]-6-[(3S)-3-methylmorpholin-4-yl]pyrimidin-2-yl]phenyl]-3-methylurea

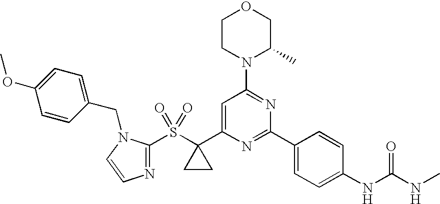

Triethylamine (0.246 mL, 1.76 mmol) was added to phenyl N-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]carbamate (240 mg, 0.35 mmol) and methylamine (0.705 mL, 1.41 mmol) in DMF (3 mL) at RT. The resulting solution was stirred at 40° C. for 30 minutes then at RT overnight. The mixture was concentrated in vacuo and chromatographed on silica, elution gradient 100% ethyl acetate, to give the desired material as a cream solid (190 mg).

NMR Spectrum: ¹H NMR (400.132 MHz, DMSO-d₆) δ 1.13 (3H, d), 1.68-1.72 (2H, m), 1.88-1.95 (2H, m), 2.67 (3H, d), 3.07-3.20 (1H, m), 3.36-3.51 (1H, m), 3.57 (1H, d), 3.68-3.79 (4H, m), 3.94 (1H, d), 4.06 (1H, s), 4.35 (1H, s), 5.21 (2H, s), 6.05 (1H, t), 6.55 (1H, s), 6.77 (2H, d), 7.05 (2H, d), 7.23 (1H, s), 7.41-7.48 (3H, m), 8.02 (2H, d), 8.72 (1H, s)

LCMS Spectrum: m/z (ESI+)(M+H)+=618; HPLC tR=2.17 min

3-Cyclopropyl-1-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]urea was made in an analogous fashion from phenyl N-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]carbamate and the appropriate amine.

| | | LCMS | Retention |
|---|---|---|---|
| Structure | NAME | MH+ | time (min) |
| 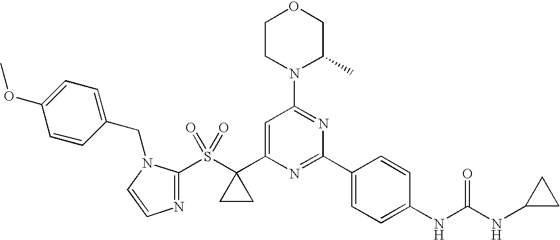 | 3-cyclopropyl-1-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]urea | 644 | 2.32 |

¹H NMR (400.132 MHz, DMSO-d₆) δ 0.38-0.45 (2H, m), 0.60-0.69 (2H, m), 1.14 (3H, d), 1.66-1.74 (2H, m), 1.88-1.94 (2H, m), 2.53-2.62 (1H, m), 3.09-3.18 (1H, m), 3.39-3.48 (1H, m), 3.57 (1H, d), 3.66-3.78 (4H, m), 3.95 (1H, d), 4.11 (1H, s), 4.35 (1H, s), 5.21 (2H, s), 6.43 (1H, s), 6.55 (1H, s), 6.77 (2H, d), 7.06 (2H, d), 7.23 (1H, s), 7.42-7.49 (3H, m), 8.02 (2H, d), 8.52 (1H, s)

The preparation of phenyl N-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]carbamate

### Phenyl N-[4-[4-[1-[1-[(4-methoxyphenyl)methyl]imidazol-2-yl]sulfonylcyclopropyl]-6-[(3S)-3-methylmorpholin-4-yl]pyrimidin-2-yl]phenyl]carbamate

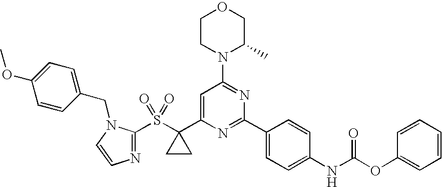

Phenyl chloroformate (0.202 mL, 1.61 mmol) was added to 4-[4-[1-[1-[(4-methoxyphenyl)methyl]imidazol-2-yl]sulfonylcyclopropyl]-6-[(3S)-3-methylmorpholin-4-yl]pyrimidin-2-yl]aniline (600 mg, 1.07 mmol) and sodium hydrogen carbonate (135 mg, 1.61 mmol) in dioxane (10 mL) at 5° C. under nitrogen. The resulting mixture was stirred at RT for 90 minutes. The reaction mixture was diluted with ethyl acetate (150 mL), and washed with water (2×100 mL). The organic layer was dried (Na₂SO₄), filtered and evaporated to afford crude product. The crude gum was triturated with a mixture of diethyl ether and isohexane to give the desired material as a beige solid which was collected by filtration and dried under vacuum (570 mg).

NMR Spectrum: ¹H NMR (400.132 MHz, DMSO-d₆) δ 1.13 (3H, d), 1.67-1.74 (2H, m), 1.90-1.95 (2H, m), 3.03-3.21 (1H, m), 3.36-3.47 (1H, m), 3.53-3.62 (1H, m), 3.64-3.76 (4H, m), 3.95 (1H, d), 4.10 (1H, s), 4.37 (1H, s), 5.23 (2H, s), 6.60 (1H, s), 6.77 (2H, d), 7.06 (2H, d), 7.23-7.33 (4H, m), 7.42-7.50 (3H, m), 7.59 (2H, d), 8.11 (2H, d), 10.41 (1H, s)

LCMS Spectrum: m/z (ESI+)(M+H)+=682; HPLC tR=2.88 min

### 4-[4-[1-[1-[(4-Methoxyphenyl)methyl]imidazol-2-yl]sulfonylcyclopropyl]-6-[(3S)-3-methylmorpholin-4-yl]pyrimidin-2-yl]aniline

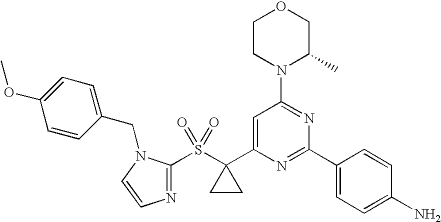

Bis(triphenylphosphine)palladium(II) chloride (42.5 mg, 0.06 mmol) was added to 2-chloro-4-[1-[1-[(4-methoxyphenyl)methyl]imidazol-2-yl]sulfonylcyclopropyl]-6-[(3S)-3-methylmorpholin-4-yl]pyrimidine (610 mg, 1.21 mmol), 4-(4,4,5,5-Tetramethyl-1,3,2-dioxaborolan-2-yl)aniline (530 mg, 2.42 mmol) and an aqueous solution of sodium carbonate (2 mL, 4.00 mmol) in a solvent mixture of DMF (2 mL), DME (4 mL), water (0.5 mL) and ethanol (0.5 mL) at RT. The atmosphere was replaced with nitrogen and the mixture stirred at 90° C. for 5 hours. The reaction mixture was diluted with ethyl acetate (100 mL), and washed with water (2×100 mL). The organic layer was dried (Na₂SO₄), filtered and evaporated to afford crude product. The crude product was purified by flash silica chromatography, elution gradient 10 to 100% ethyl acetate in isohexane, to give the desired material as a white solid (600 mg).

NMR Spectrum: ¹H NMR (400.132 MHz, DMSO-d₆) δ 1.11 (3H, d), 1.65-1.69 (2H, m), 1.88-1.92 (2H, m), 3.04-3.15 (1H, m), 3.41 (1H, td), 3.56 (1H, d), 3.67-3.75 (4H, m), 3.93 (1H, d), 4.06 (1H, s), 4.31 (1H, s), 5.16 (2H, s), 5.54 (2H, s), 6.44 (1H, s), 6.57 (2H, d), 6.79 (2H, d), 7.06 (2H, d), 7.22 (1H, s), 7.43 (1H, s), 7.87 (2H, d)

### 2-Chloro-4-[1-[1-[(4-methoxyphenyl)methyl]imidazol-2-yl]sulfonylcyclopropyl]-6-[(3S)-3-methylmorpholin-4-yl]pyrimidine

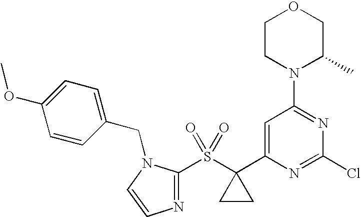

An aqueous solution of sodium hydroxide (10 mL, 186.4 mmol) was added to 2-chloro-4-[[1-[(4-methoxyphenyl)methyl]imidazol-2-yl]sulfonylmethyl]-6-[(3S)-3-methylmorpholin-4-yl]pyrimidine (1.1 g, 2.30 mmol), tetraethylammonium bromide (0.097 g, 0.46 mmol), and 1,2-dibromoethane (2.38 mL, 27.62 mmol) in DCM (20 mL) at RT under a nitrogen atmosphere. The reaction was stirred at RT for 4 hours. The reaction mixture was diluted with DCM (50 mL), the phases separated and the organic layer dried (Na₂SO₄), filtered and evaporated. The residue was purified by flash silica chromatography, elution gradient 10 to 75% ethyl acetate in isohexane, to give the desired material as a white solid (0.77 g).

NMR Spectrum: ¹H NMR (400.132 MHz, DMSO-d₆) δ 1.10 (4H, d), 1.64 (3H, m), 1.85-1.89 (2H, m), 3.03-3.15 (1H, m), 3.33-3.42 (1H, m), 3.50 (1H, d), 3.68 (1H, d), 3.74 (3H, s), 3.87-3.93 (2H, m), 4.06 (1H, s), 5.33 (2H, s), 6.57 (1H, s), 6.87 (2H, d), 7.17 (2H, d), 7.22 (1H, s), 7.59 (1H, s)

LCMS Spectrum: m/z (ESI+)(M+H)+=504; HPLC tR=2.35 min

### 2-Chloro-4-[[1-[(4-methoxyphenyl)methyl]imidazol-2-yl]sulfonylmethyl]-6-[(3I)-3-methylmorpholin-4-yl]pyrimidine

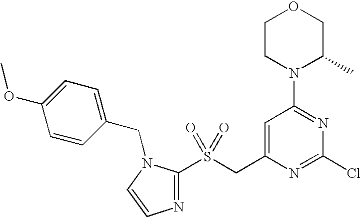

4-Methoxybenzyl chloride (0.470 mL, 3.46 mmol) was added to 2-chloro-4-(1H-imidazol-2-ylsulfonylmethyl)-6-[(3S)-3-methylmorpholin-4-yl]pyrimidine (1.18 g, 3.30 mmol) and potassium carbonate (0.501 g, 3.63 mmol) in DMF (30 mL) at RT under a nitrogen atmosphere. The resulting mixture was stirred at 75° C. for 1 hour then allowed to cool and diluted with ethyl acetate (100 mL). The mixture was washed with water (2×50 mL), the organic layer dried (Na₂SO₄), filtered and evaporated to afford crude product. The crude product was purified by flash silica chromatography, elution gradient 10 to 70% ethyl acetate in isohexane, to give the desired material as a colourless gum (1.27 g).

NMR Spectrum: ¹H NMR (400.132 MHz, DMSO-d₆) δ 1.13 (3H, d), 3.10-3.19 (1H, m), 3.35-3.45 (1H, m), 3.54 (1H, d), 3.70 (1H, d), 3.74 (3H, s), 3.87-3.96 (2H, m), 4.14 (1H, s), 4.68 (2H, s), 5.36 (2H, s), 6.55 (1H, s), 6.88 (2H, d), 7.15-7.28 (3H, m), 7.57 (1H, s)

LCMS Spectrum: m/z (ESI+)(M+H)+=478; HPLC tR=2.26 min

### 2-Chloro-4-(1H-imidazol-2-ylsulfonylmethyl)-6-[(3S)-3-methylmorpholin-4-yl]pyrimidine

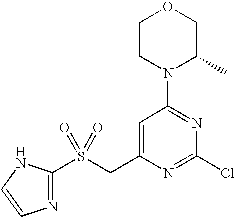

3-Chloroperoxybenzoic acid (2.62 g, 15.19 mmol) was added to 2-chloro-4-(1H-imidazol-2-ylsulfanylmethyl)-6-[(3S)-3-methylmorpholin-4-yl]pyrimidine (2.25 g, 6.91 mmol) in DCM (100 mL) at RT under a nitrogen atmosphere. The resulting solution was stirred at RT for 2 hours. The reaction mixture was diluted with DCM (100 mL), and washed sequentially with an aqueous 10% solution of sodium metabisulphite (200 mL), and a saturated solution of sodium hydrogen carbonate (200 mL). The organic layer was dried (Na₂SO₄), filtered and evaporated to afford crude product. The crude solid was triturated with a mixture of diethyl 1o ether and isohexane to give the desired material as a white solid that was collected by filtration and dried under vacuum (1.8 g).

NMR Spectrum: ¹H NMR (400.132 MHz, DMSO-d₆) δ 1.16 (3H, d), 3.06-3.21 (1H, m), 3.35-3.46 (1H, m), 3.55 (1H, d), 3.71 (1H, d), 3.83-3.97 (2H, m), 4.16 (1H, s), 4.66 (2H, s), 6.52 (1H, s), 7.35 (2H, s), 13.65(1H,s).

LCMS Spectrum: m/z (ESI+)(M+H)+=358; HPLC tR=0.87 min

### 2-Chloro-4-(1H-imidazol-2-ylsulfanylmethyl)-6-[(3S)-3-methylmorpholin-4-yl]pyrimidine

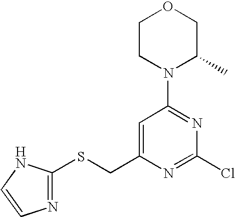

DIPEA (2.94 mL, 16.97 mmol) was added to 1H-imidazole-2-thiol (1.246 g, 12.44 mmol), in acetonitrile (50 mL) at RT under a nitrogen atmosphere. The resulting solution was stirred at RT for 20 minutes. 2-Chloro-4-(iodomethyl)-6-[(3S)-3-methylmorpholin-4-yl]pyrimidine (4 g, 11.31 mmol) was added and the mixture stirred for 1 hour. The reaction mixture was diluted with ethyl acetate (300 mL), and washed with water (150 mL). The organic layer was dried (MgSO₄), filtered and evaporated to afford crude product. The crude product was purified by flash silica chromatography, elution gradient 0 to 4% methanol in ethyl acetate, to give the desired material as a white solid (2.80 g).

NMR Spectrum: ¹H NMR (400.132 MHz, DMSO-d₆) δ 1.13 (3H, d), 3.11 (1H, dd), 3.34-3.45 (1H, m), 3.54 (1H, dd), 3.69 (1H, d), 3.87-3.95 (2H, m), 4.06 (2H, s), 4.17 (1H, s), 6.52 (1H, s), 6.96 (1H, s), 7.17 (1H, s), 12.35 (1H,s).

LCMS Spectrum: m/z (ESI+)(M+H)+=326; HPLC tR=1.41 min

The preparation of 2-chloro-4-(iodomethyl)-6-[(3S)-3-methylmorpholin-4-yl]pyrimidine was described earlier.

### EXAMPLE 41

### 3-(2-Cyanoethyl)-1-[4-[4-[(3S)-3-methylmorpholin-4-yl]-6-(1-methylsulfonylcyclopropyl)pyrimidin-2-yl]phenyl]thiourea

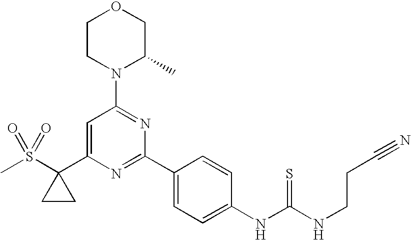

A solution of 1,1′-thiocarbonyldiimidazole (50 mg, 0.28 mmol) in DCM (1 mL) was added to a suspension of 4-[4-[(3S)-3-methylmorpholin-4-yl]-6-(1-methylsulfonylcyclopropyl)pyrimidin-2-yl]aniline (75 mg, 0.19 mmol) in DCM (2 mL) and THF (1 mL). The reaction mixture was stirred at RT for 1 hour before addition of 3-aminopropionitrile (91 mg, 1.30 mmol) and triethylamine (0.026 mL, 0.19 mmol). Stirring was continued at RT overnight. The reaction was incomplete and significant insoluble material was observed so DMF (1 mL) was added and stirring continued for a further 1 hour. The reaction was still incomplete, so the reaction mixture was transferred to a microwave tube, sealed, heated to 100° C. in the microwave reactor and held for 10 minutes. The reaction was still incomplete, so further 3-aminopropionitrile (91 mg, 1.30 mmol) was added and the reaction mixture stirred at RT for 2-3 hours. The reaction mixture was evaporated and residue purified by preparative HPLC, using decreasingly polar mixtures of water (containing 1% ammonia) and acetonitrile as eluents, to give the desired material as a white solid (29 mg).

NMR Spectrum: ¹H NMR (400.132 MHz, DMSO-d₆) δ 1.24 (3H, d), 1.55-1.58 (2H, m), 1.67-1.69 (2H, m), 2.87 (2H, t), 3.19-3.26 (1H, m), 3.27 (3H, s), 3.49 (1H, td), 3.64 (1H, dd), 3.74-3.78 (3H, m), 3.98 (1H, dd), 4.18-4.25 (1H, m), 4.56-4.63 (1H, m), 6.82 (1H, s), 7.57 (2H, d), 8.16 (1H, s), 8.26-8.30 (2H, m), 9.98 (1H, s)

LCMS Spectrum: m/z (ESI+)(M+H)+=501; HPLC tR=1.99 min mTOR Kinase Assay (Echo): 0.0308 μM

The preparation of 4-[4-[(3S)-3-methylmorpholin-4-yl]-6-(1-methylsulfonylcyclopropyl)pyrimidin-2-yl]aniline was described previously.

### EXAMPLE 42

### 3-Cyclopropyl-1-[4-[4-[1-[(4-methyl-1,3-thiazol-2-yl)sulfonyl]cyclopropyl]-6-morpholin-4-ylpyrimidin-2-yl]phenyl]urea

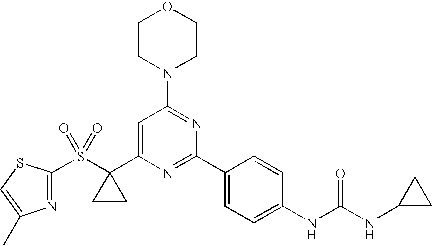

Triethylamine (0.119 mL, 0.85mmol) was added to a solution of phenyl N-[4-[4-[1-[(4-methyl-1,3-thiazol-2-yl)sulfonyl]cyclopropyl]-6-morpholin-4-ylpyrimidin-2-yl]phenyl]carbamate (100 mg, 0.17 mmol) and cyclopropylamine (0.059 mL, 0.85 mmol) in NMP (2 mL) and the resulting solution stirred at ambient temperature for 18 hours. The crude product was purified by preparative HPLC to give the desired material as a white solid (80 mg).

NMR Spectrum: ¹H NMR (400.132 MHz, DMSO-d₆) δ 0.40-0.44 (2H, m), 0.62-0.67 (2H, m), 1.75-1.78 (2H, m), 1.94-1.97 (2H, m), 2.49 (3H, s), 2.54-2.58 (1H, m), 3.70 (8H, s), 6.40 (1H, s), 6.82 (1H, s), 7.42 (2H, d), 7.84 (1H, s), 7.87 (2H, d), 8.52 (1H, s)

LCMS Spectrum: m/z (ESI+)(M+H)+=541; HPLC tR=2.15 min.

mTOR Kinase Assay (Echo): 0.000705 μM

The compounds below were prepared in an analogous fashion using the appropriate amine.

| | | | LCMS | Retention |
|---|---|---|---|---|
| Example | Structure | NAME | MH+ | time (min) |
| 42a | 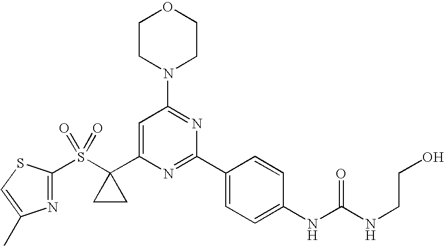 | 3-(2-hydroxyethyl)-1-[4-[4-[1-[(4-methyl-1,3-thiazol-2-yl)sulfonyl]cyclopropyl]-6-morpholin-4-ylpyrimidin-2-yl]phenyl]urea | 545 | 1.79 |
| 42b | 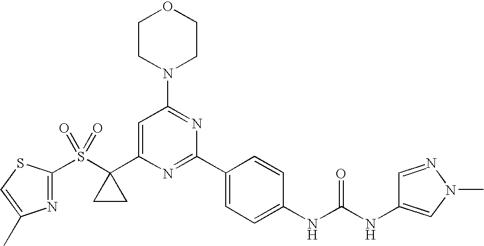 | 3-(1-methylpyrazol-4-yl)-1-[4-[4-[1-[(4-methyl-1,3-thiazol-2-yl)sulfonyl]cyclopropyl]-6-morpholin-4-ylpyrimidin-2-yl]phenyl]urea | 581 | 2.01 |
| 42c | 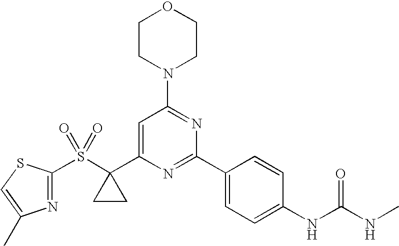 | 3-methyl-1-[4-[4-[1-[(4-methyl-1,3-thiazol-2-yl)sulfonyl]cyclopropyl]-6-morpholin-4-ylpyrimidin-2-yl]phenyl]urea | 515 | 1.96 |
| 42d | 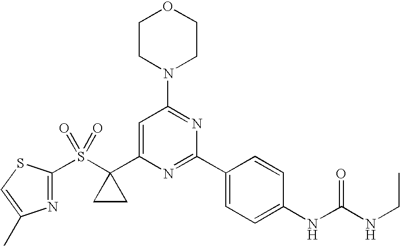 | 1-ethyl-3-[4-[4-[1-[(4-methyl-1,3-thiazol-2-yl)sulfonyl]cyclopropyl]-6-morpholin-4-ylpyrimidin-2-yl]phenyl]urea | 529 | 2.14 |
| 42e | 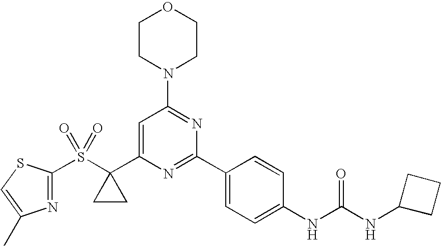 | 3-cyclobutyl-1-[4-[4-[1-[(4-methyl-1,3-thiazol-2-yl)sulfonyl]cyclopropyl]-6-morpholin-4-ylpyrimidin-2-yl]phenyl]urea | 555 | 1.95 |
| 42f | 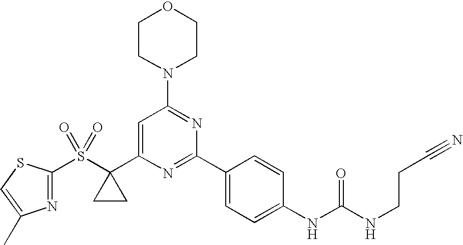 | 3-(2-cyanoethyl)-1-[4-[4-[1-[(4-methyl-1,3-thiazol-2-yl)sulfonyl]cyclopropyl]-6-morpholin-4-ylpyrimidin-2-yl]phenyl]urea | 554 | 1.63 |
| 42g | 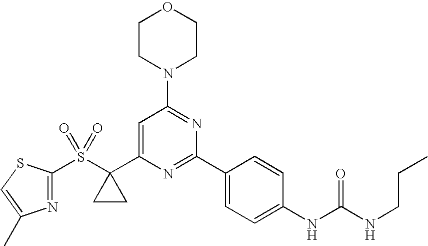 | 3-[4-[4-[1-[(4-methyl-1,3-thiazol-2-yl)sulfonyl]cyclopropyl]-6-morpholin-4-ylpyrimidin-2-yl]phenyl]-1-propylurea | 543 | 2.13 |
| 42h | 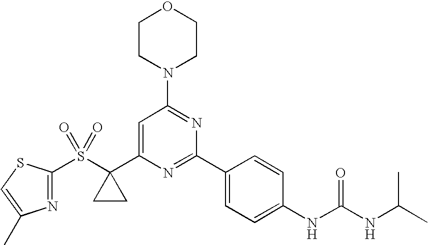 | 1-[4-[4-[1-[(4-methyl-1,3-thiazol-2-yl)sulfonyl]cyclopropyl]-6-morpholin-4-ylpyrimidin-2-yl]phenyl]-3-propan-2-ylurea | 543 | 2.13 |

### EXAMPLE 42a

¹H NMR (400.132 MHz, DMSO-d₆) δ 1.75-1.78 (2H, m), 1.94-1.97 (2H, m), 2.48 (3H, s), 3.15-3.20 (2H, m), 3.44-3.48 (2H, m), 3.70 (8H, s), 4.72 (1H, t), 6.23 (1H, t), 6.82 (1H, s), 7.40 (2H, d), 7.84 (1H, s), 7.87 (2H, d), 8.78 (1H, s).

mTOR Kinase Assay (Echo): 0.00261 μM

### EXAMPLE 42b

¹H NMR (400.132 MHz, DMSO-d₆) δ 1.75-1.79 (2H, m), 1.94-1.98 (2H, m), 2.50 (3H, s), 3.70 (8H, s), 3.79 (3H, s), 6.83 (1H, s), 7.38 (1H, s), 7.46 (2H, d), 7.77 (1H, s), 7.85 (1H, s), 7.90 (2H, d), 8.36 (1H, s), 8.82 (1H, s).

### EXAMPLE 42c

¹H NMR (400.132 MHz, DMSO-d₆) δ 1.75-1.78 (2H, m), 1.94-1.97 (2H, m), 2.49 (3H, s), 2.66 (3H, d), 3.70 (8H, s), 6.04 (1H, q), 6.82 (1H, s), 7.41 (2H, d), 7.84 (1H, s), 7.86 (2H, d), 8.72 (1H, s).

mTOR Kinase Assay (Echo): 0.0036 μM

### EXAMPLE 42d

¹H NMR (400.132 MHz, DMSO-d₆) δ 1.07 (3H, t), 1.75-1.78 (2H, m), 1.94-1.97 (2H, m), 2.49 (3H, s), 3.09-3.16 (2H, m), 3.70 (8H, s), 6.14 (1H, t), 6.82 (1H, s), 7.41 (2H, d), 7.84 (1H, s), 7.86 (2H, d), 8.64 (1H, s).

mTOR Kinase Assay (Echo): 0.000425 μM

### EXAMPLE 42e

¹H NMR (400.132 MHz, DMSO-d₆) δ 1.57-1.66 (2H, m), 1.75-1.78 (2H, m), 1.81-1.91 (2H, m), 1.94-1.97 (2H, m), 2.18-2.25 (2H, m), 2.48 (3H, s), 3.70 (8H, s), 4.09-4.19 (1H, m), 6.43 (1H, d), 6.82 (1H, s), 7.39 (2H, d), 7.84 (1H, s), 7.86 (2H, d), 8.54 (1H, s).

mTOR Kinase Assay (Echo): 0.00257 μM

### EXAMPLE 42f

¹H NMR (400.132 MHz, DMSO-d₆) δ 1.75-1.78 (2H, m), 1.94-1.97 (2H, m), 2.49 (3H, s), 2.70 (2H, t), 3.34-3.39 (2H, m), 3.70 (8H, s), 6.52 (1H, t), 6.83 (1H, s), 7.43 (2H, d), 7.84 (1H, s), 7.88 (2H, d), 8.92 (1H, s).

mTOR Kinase Assay (Echo): 0.00264 μM

### EXAMPLE 42g

¹H NMR (400.132 MHz, DMSO-d₆) δ 0.89 (3H, t), 1.41-1.50 (2H, m), 1.75-1.78 (2H, m), 1.94-1.97 (2H, m), 2.49 (3H, s), 3.04-3.09 (2H, m), 3.70 (8H, s), 6.18 (1H, t), 6.82 (1H, s), 7.40 (2H, d), 7.84 (1H, s), 7.86 (2H, d), 8.63 (1H, s).

mTOR Kinase Assay (Echo): 0.00401 μM

### EXAMPLE 42h

¹H NMR (400.132 MHz, DMSO-d₆) δ 1.11 (6H, d), 1.75-1.78 (2H, m), 1.94-1.97 (2H, m), 2.48 (3H, s), 3.70 (8H, s), 3.73-3.81 (1H, m), 6.03 (1H, d), 6.82 (1H, s), 7.39 (2H, d), 7.84 (1H, s), 7.86 (2H, d), 8.51 (1H, s).

mTOR Kinase Assay (Echo): 0.00412 μM

The preparation of phenyl N-[4-[4-[1-[(4-methyl-1,3-thiazol-2-yl)sulfonyl]cyclopropyl]-6-morpholin-4-ylpyrimidin-2-yl]phenyl]carbamate is described below:

### Phenyl N-[4-[4-[1-[(4-methyl-1,3-thiazol-2-yl)sulfonyl]cyclopropyl]-6-morpholin-4-ylpyrimidin-2-yl]phenyl]carbamate

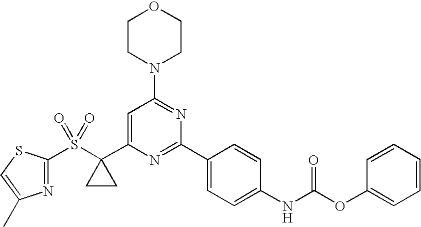

Phenyl chloroformate (0.664 mL, 5.29 mmol) was added to 4-[4-[1-[(4-methyl-1,3-thiazol-2-yl)sulfonyl]cyclopropyl]-6-morpholin-4-ylpyrimidin-2-yl]aniline (2.2 g, 4.81 mmol) and sodium hydrogen carbonate (0.606 g, 7.21 mmol) in dioxane (100 mL) at 10° C. under a nitrogen atmosphere. The resulting mixture was stirred at 10° C. for 2 hours. The reaction mixture was diluted with ethyl acetate (200 mL), and washed sequentially with water (100 mL) and saturated brine (100 mL). The organic layer was dried (MgSO₄), filtered and evaporated to afford crude product as a gum (2.83 g).

NMR Spectrum: ¹H NMR (400.132 MHz, DMSO-d₆) δ 1.76-1.80 (2H, m), 1.94-1.97 (2H, m), 2.48 (3H, s), 3.70 (8H, s), 6.87 (1H, s), 7.24-7.30 (3H, m), 7.43-7.47 (2H, m), 7.55 (2H, d), 7.85 (1H, s), 7.96 (2H, d), 10.45 (1H, s)

LCMS Spectrum: m/z (ESI+) (M+H)+=578; HPLC tR=2.88 min.

### 4-[4-[1-[(4-Methyl-1,3-thiazol-2-yl)sulfonyl]cyclopropyl]-6-morpholin-4-ylpyrimidin-2-yl]aniline

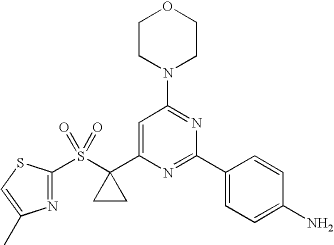

Bis(triphenylphosphine)palladium (II) chloride (0.256 g, 0.37 mmol) was added to 2-chloro-4-[1-[(4-methyl-1,3-thiazol-2-yl)sulfonyl]cyclopropyl]-6-morpholin-4-ylpyrimidine (2.93 g, 7.31 mmol), 4-(4,4,5,5-tetramethyl-1,3,2-dioxaborolan-2-yl)aniline (2.082 g, 9.50 mmol) and 2M aqueous sodium carbonate (13.16 mL, 26.31 mmol) in a solvent mixture of DMF (15 mL), water (37.5 mL), ethanol (15 mL) and DME (15 mL) at RT under an atmosphere of nitrogen. The resulting mixture was stirred at 80° C. for 16 hours. The cooled reaction mixture was diluted with ethyl acetate (100 mL) and washed sequentially with water (100 mL) and saturated brine (100 mL). The organic layer was dried (MgSO₄), filtered and evaporated to afford crude product which was purified by flash silica chromatography, elution gradient 0 to 75% ethyl acetate in DCM, to give the desired material as a cream solid (2.2 g).

NMR Spectrum: ¹H NMR (400.132 MHz, DMSO-d₆) δ 1.72-1.75 (2H, m), 1.91-1.94 (2H, m), 2.49 (3H, s), 3.62-3.71 (8H, m), 5.57 (2H, s), 6.50 (2H, d), 6.71 (1H, s), 7.68 (2H, d), 7.84 (1H, s)

LCMS Spectrum: m/z (ESI+) (M+H)+=458; HPLC tR=2.21 min.

### 2-Chloro-4-[1-[(4-methyl-1,3-thiazol-2-yl)sulfonyl]cyclopropyl]-6-morpholin-4-ylpyrimidine

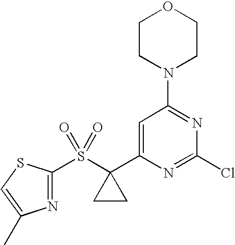

1,2-Dibromoethane (0.230 mL, 2.67 mmol) was added to 2-chloro-4-[(4-methyl-1,3-thiazol-2-yl)sulfonylmethyl]-6-morpholin-4-ylpyrimidine (500 mg, 1.33 mmol), 40% sodium hydroxide solution (1.3 mL,13 mmol) and tetrabutylammonium bromide (86 mg, 0.27 mmol) in toluene (10 mL) at RT. The resulting solution was stirred at 60° C. for 3 hours. The cooled reaction mixture was evaporated to dryness and redissolved in ethyl acetate (50 mL), and washed sequentially with water (25 mL) and saturated brine (25 mL). The organic layer was dried (MgSO₄), filtered and evaporated to give the desired material as a pale brown gum (528 mg).

NMR Spectrum: ¹H NMR (400.13 MHz, CDCl₃) δ 1.76-1.79 (2H, m), 2.09-2.12 (2H, m), 2.52 (3H, s), 3.65-3.71 (4H, m), 3.77-3.79 (4H, m), 7.29 (1H, s), 7.30 (1H, s)

LCMS Spectrum: m/z (ESI+) (M+H)+=401; HPLC tR=2.04 min.

### 2-Chloro-4-[(4-methyl-1,3-thiazol-2-yl)sulfonylmethyl]-6-morpholin-4-ylpyrimidine

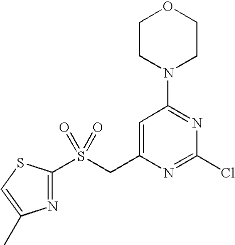

A solution of morpholine (0.994 g, 11.41 mmol) in DCM (25 mL) was added dropwise to a stirred solution of 2,4-dichloro-6-[(4-methyl-1,3-thiazol-2-yl)sulfonylmethyl]pyrimidine (3.7 g, 11.41 mmol) and triethylamine (1.155 g, 11.41 mmol) in DCM (50 mL). The resulting solution was stirred at RT for 18 hours. The reaction mixture was washed three times with water and the organic layer dried (MgSO₄), filtered and evaporated to afford crude product. The crude product was purified by flash silica chromatography, elution gradient 0 to 50% ethyl acetate in DCM, to give the desired material as a yellow solid (2.84 g).

NMR Spectrum: ¹H NMR (400.13 MHz, CDCl₃) δ 2.57 (3H, s), 3.58-3.69 (8H, m), 4.56 (2H, s), 6.58 (1H, s), 7.30 (1H, s)

LCMS Spectrum: m/z (ESI+) (M+H)+=375; HPLC tR=2.14 min.

The preparation of 2,4-dichloro-6-[(4-methyl-1,3-thiazol-2-yl)sulfonylmethyl]pyrimidine was described previously.

### EXAMPLE 43

### 3-Cyclopropyl-1-[4-[4-[1-(3-hydroxypropylsulfonyl)cyclobutyl]-6-[(3S)-3-methylmorpholin-4-yl]pyrimidin-2-yl]phenyl]urea

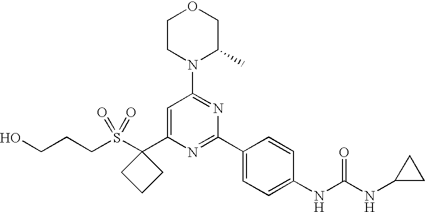

Cyclopropylamine (0.122 mL, 1.76 mmol) was added in one portion to a stirred solution of phenyl N-[4-[4-[1-(3-hydroxypropylsulfonyl)cyclobutyl]-6-[(3S)-3-methylmorpholin-4-yl]pyrimidin-2-yl]phenyl]carbamate (0.2 g, 0.35 mmol) and triethylamine (0.148 mL, 1.06 mmol) in DMA (35.3 mL) at RT. The resulting solution was stirred at 50° C. for 24 hours. The reaction mixture was then concentrated, and the crude product was purified by flash silica chromatography, elution gradient 0 to 5% methanol in DCM, to give a clear oil which was then triturated with diethyl ether to give the desired material as a white solid (0.126 g).

NMR Spectrum: ¹H NMR (400.13 MHz, CDCl₃) δ 0.60-0.67 (2H, m), 0.74-0.83 (2H, m), 1.33 (3H, d), 1.94-2.01 (3H, m), 2.16-2.25 (2H, m), 2.57-2.63 (1H, m), 2.80-2.88 (2H, m), 2.98-3.01 (2H, m), 3.09-3.16 (2H, m), 3.28-3.35 (1H, m), 3.56-3.62 (3H, m), 3.71-3.75 (1H, m), 3.81 (1H, d), 4.01-4.05 (1H, m), 4.16 (1H, d), 4.46 (1H, d), 5.30 (1H, s), 6.55 (1H, s), 7.31 (1H, s), 7.48 (2H, d), 8.32 (2H, d).

LCMS Spectrum: m/z (ESI+)(M+H)+=530; HPLC tR=2.09 min.

mTOR Kinase Assay (Echo): 0.00539 μM

The compounds below were prepared in an analogous fashion from either phenyl N-[4-[4-[1-(3-hydroxypropylsulfonyl)cyclobutyl]-6-[(3S)-3-methylmorpholin-4-yl]pyrimidin-2-yl]phenyl]carbamate or phenyl N-[4-[4-[1-(3-hydroxypropylsulfonyl)cyclopentyl]-6-[(3S)-3-methylmorpholin-4-yl]pyrimidin-2-yl]phenyl]carbamate using the appropriate amine.

| | | | LCMS | Retention |
|---|---|---|---|---|
| Example | Structure | NAME | MH+ | time (min) |
| 43a | 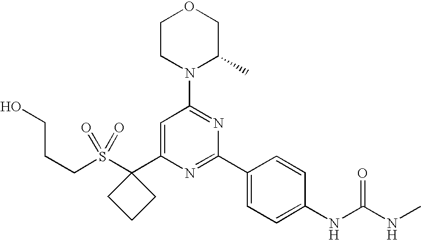 | 1-[4-[4-[1-(3-hydroxypropylsulfonyl)cyclobu-tyl]-6-[(3S)-3-methylmorpholin-4-yl]pyrimidin-2-yl]phenyl]-3-methylurea | 504 | 1.93 |
| 43b | 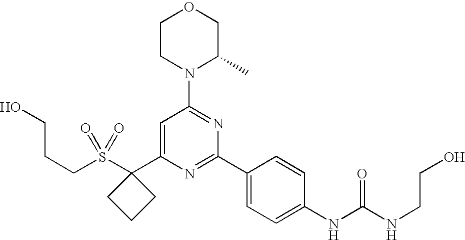 | 3-(2-hydroxyethyl)-1-[4-[4-[1-(3-hydroxypropylsulfonyl)cyclobu-tyl]-6-[(3S)-3-methylmorpholin-4-yl]pyrimidin-2-yl]phenyl]urea | 534 | 1.83 |
| 43c | 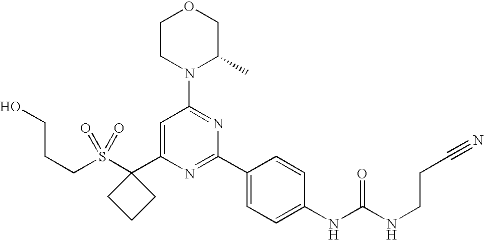 | 3-(2-cyanoethyl)-1-[4-[4-[1-(3-hydroxypropylsulfonyl)cyclobu-tyl]-6-[(3S)-3-methylmorpholin-4-yl]pyrimidin-2-yl]phenyl]urea | 543 | 2.00 |
| 43d | 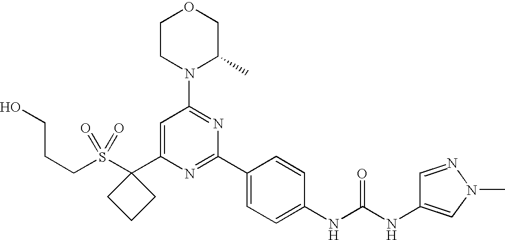 | 1-[4-[4-[1-(3-hydroxypropylsulfonyl)cyclobu-tyl]-6-[(3S)-3-methylmorpholin-4-yl]pyrimidin-2-yl]phenyl]-3-(1-methylpyrazol-4-yl)urea | 570 | 1.98 |
| 43e | 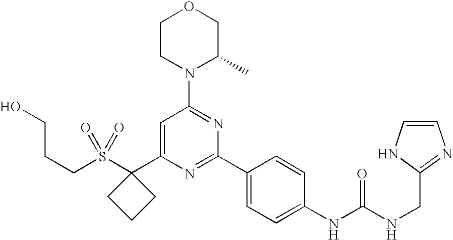 | 1-[4-[4-[1-(3-hydroxypropylsulfonyl)cyclobu-tyl]-6-[(3S)-3-methylmorpholin-4-yl]pyrimidin-2-yl]phenyl]-3-(1H-imidazol-2-ylmethyl)urea | 570 | 1.93 |
| 43f | 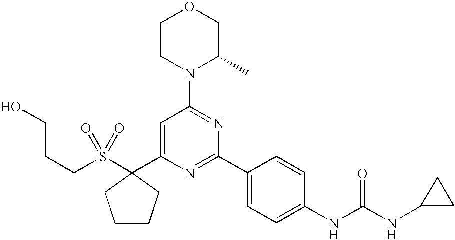 | 3-cyclopropyl-1-[4-[4-[1-(3-hydroxypropyl-sulfonyl)cyclopen-tyl]-6-[(3S)-3-methylmorpholin-4-yl]pyrimidin-2-yl]phenyl]urea | 544 | 2.21 |
| 43g | 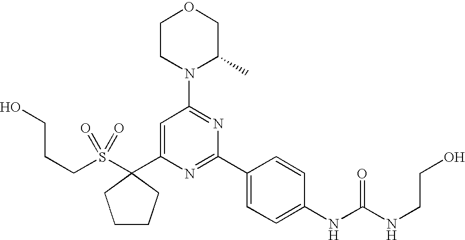 | 3-(2-hydroxyethyl)-1-[4-[4-[1-(3-hydroxypropyl-sulfonyl)cyclopen-tyl]-6-[(3S)-3-methylmorpholin-4-yl]pyrimidin-2-yl]phenyl]urea | 548 | 1.91 |
| 43h | 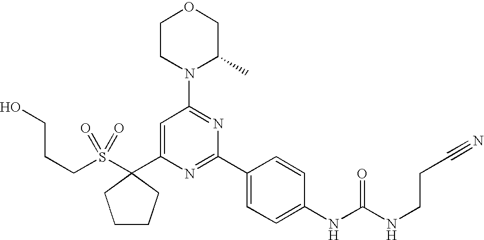 | 3-(2-cyanoethyl)-1-[4-[4-[1-(3-hydroxypropyl-sulfonyl)cyclopen-tyl]-6-[(3S)-3-methylmorpholin-4-yl]pyrimidin-2-yl]phenyl]urea | 557 | 2.12 |
| 43i | 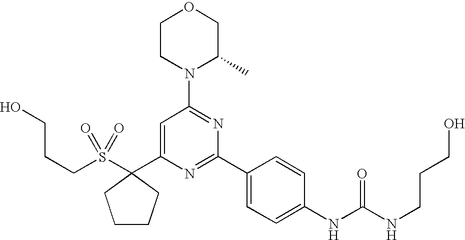 | 3-(3-hydroxypropyl)-1-[4-[4-[1-(3-hydroxypropyl-sulfonyl)cyclopen-tyl]-6-[(3S)-3-methylmorpholin-4-yl]pyrimidin-2-yl]phenyl]urea | 562 | 1.94 |
| 43j* | 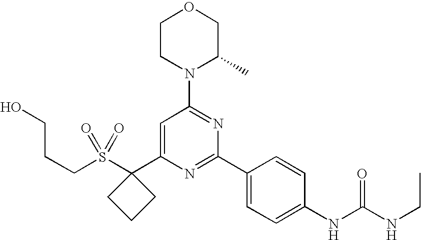 | 1-ethyl-3-[4-[4-[1-(3-hydroxypropylsulfonyl)cyclobu-tyl]-6-[(3S)-3-methylmorpholin-4-yl]pyrimidin-2-yl]phenyl]urea | 518 | 1.89 |
| 43k* | 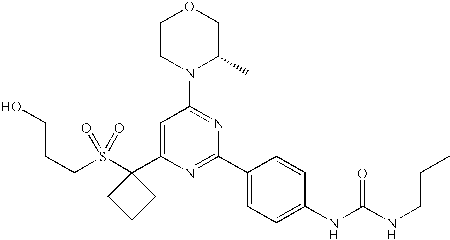 | 3-[4-[4-[1-(3-hydroxypropylsulfonyl)cyclobu-tyl]-6-[(3S)-3-methylmorpholin-4-yl]pyrimidin-2-yl]phenyl]-1-propylurea | 532 | 2.04 |
| 43l* | 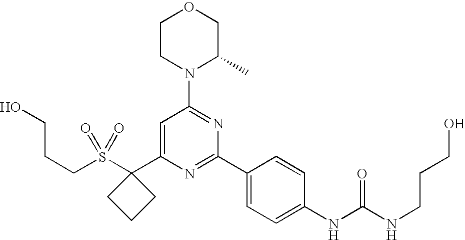 | 3-(3-hydroxypropyl)-1-[4-[4-[1-(3-hydroxypropylsulfonyl)cyclobu-tyl]-6-[(3S)-3-methylmorpholin-4-yl]pyrimidin-2-yl]phenyl]urea | 548 | 1.68 |
| 43m* | 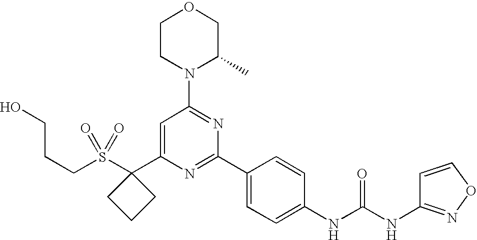 | 1-[4-[4-[1-(3-hydroxypropylsulfonyl)cyclobu-tyl]-6-[(3S)-3-methylmorpholin-4-yl]pyrimidin-2-yl]phenyl]-3-(1,2-oxazol-3-yl)urea | 557 | 2.04 |
| 43n* | 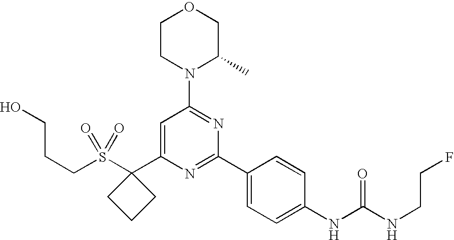 | 3-(2-fluoroethyl)-1-[4-[4-[1-(3-hydroxypropylsulfonyl)cyclobu-tyl]-6-[(3S)-3-methylmorpholin-4-yl]pyrimidin-2-yl]phenyl]urea | 536 | 1.88 |
| 43o* | 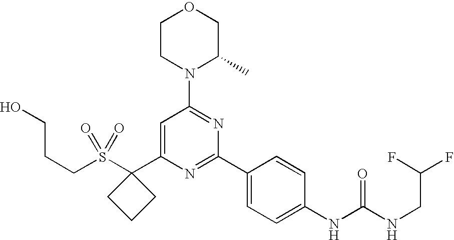 | 3-(2,2-difluoroethyl)-1-[4-[4-[1-(3-hydroxypropylsulfonyl)cyclobu-tyl]-6-[(3S)-3-methylmorpholin-4-yl]pyrimidin-2-yl]phenyl]urea | 554 | 2.00 |
| 43p* | 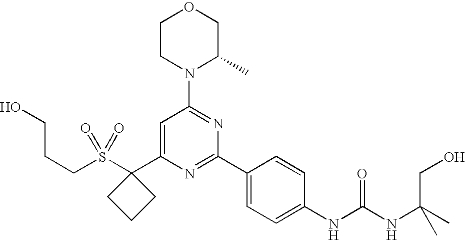 | 3-(1-hydroxy-2-methylpropan-2-yl)-1-[4-[4-[1-(3-hydroxypropylsulfonyl)cyclobu-tyl]-6-[(3S)-3-methylmorpholin-4-yl]pyrimidin-2-yl]phenyl]urea | 562 | 1.88 |
| 43q* | 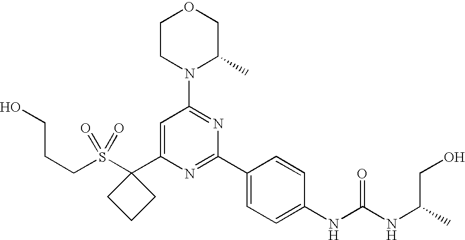 | 3-[(2S)-1-hydroxypropan-2-yl]-1-[4-[4-[1-(3-hydroxypropylsulfonyl)cyclobu-tyl]-6-[(3S)-3-methylmorpholin-4-yl]pyrimidin-2-yl]phenyl]urea | 548 | 1.72 |
| 43r* | 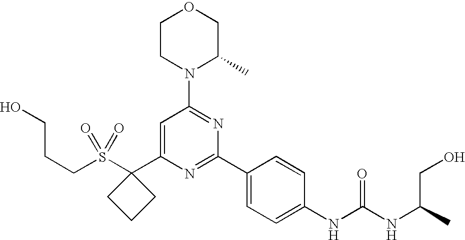 | 3-[(2R)-1-hydroxypropan-2-yl]-1-[4-[4-[1-(3-hydroxypropylsulfonyl)cyclobu-tyl]-6-[(3S)-3-methylmorpholin-4-yl]pyrimidin-2-yl]phenyl]urea | 548 | 1.71 |
| 43s* | 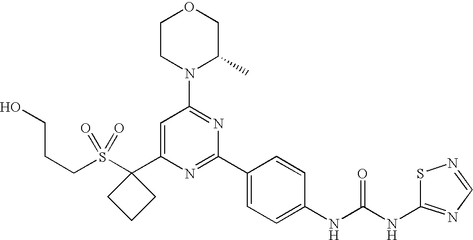 | 1-[4-[4-[1-(3-hydroxypropylsulfonyl)cyclobu-tyl]-6-[(3S)-3-methylmorpholin-4-yl]pyrimidin-2-yl]phenyl]-3-(1,2,4-thiadiazol-5-yl)urea | 574 | 1.28 |
| 43t* | 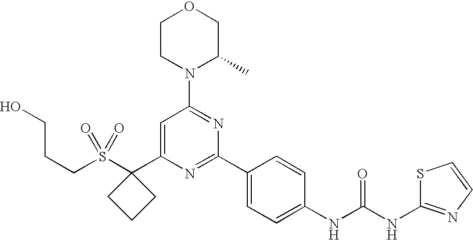 | 1-[4-[4-[1-(3-hydroxypropylsulfonyl)cyclobu-tyl]-6-[(3S)-3-methylmorpholin-4-yl]pyrimdiin-2-yl]phenyl]-3-(1,3-thiazol-2-yl)urea | 573 | 2.05 |

*The reactions were stirred in NMP at 70° C. for 2.5 hours

### EXAMPLE 43a

¹H NMR (400.13 MHz, CDCl₃) δ 1.30 (3H, d), 1.94-1.99 (3H, m), 2.14-2.23 (1H, m), 2.73 (3H, d), 2.79-2.90 (3H, m), 2.96-3.04 (2H, m), 3.06-3.13 (2H, m), 3.24-3.32 (1H, m), 3.54-3.59 (3H, m), 3.68-3.72 (1H, m), 3.78 (1H, d), 3.98-4.02 (1H, m), 4.10-4.15 (1H, m), 4.43 (1H, s), 5.49 (1H, q), 6.52 (1H, s), 7.41 (2H, d), 7.62 (1H, s), 8.29 (2H, d).

mTOR Kinase Assay (Echo): 0.00479 μM

### EXAMPLE 43b

¹H NMR (400.13 MHz, CDCl₃) δ 1.28 (3H, d), 1.90-2.00 (3H, m), 2.08-2.22 (2H, m), 2.79-2.88 (2H, m), 2.94-3.09 (4H, m), 3.24-3.33 (3H, m), 3.45-3.56 (6H, m), 3.68 (1H, d), 3.76 (1H, d), 3.98 (1H, d), 4.11 (1H, d), 4.42 (1H, s), 5.84 (1H, t), 6.50 (1H, s), 7.39 (2H, d), 7.89 (1H, s), 8.27 (2H, d).

mTOR Kinase Assay (Echo): 0.00751 μM

### EXAMPLE 43c

¹H NMR (400.13 MHz, CDCl₃) δ 1.31 (3H, d), 1.93-2.04 (4H, m), 2.16-2.25 (1H, m), 2.55-2.60 (2H, m), 2.79-2.88 (2H, m), 2.97-3.06 (2H, m), 3.09-3.15 (2H, m), 3.26-3.33 (1H, m), 3.43-3.49 (2H, m), 3.53-3.63 (3H, m), 3.69-3.73 (1H, m), 3.79 (1H, d), 3.99-4.03 (1H, m), 4.15 (1H, d), 4.44 (1H, s), 5.87 (1H, t), 6.55 (1H, s), 7.42 (2H, d), 7.54 (1H, s), 8.31 (2H, d).

mTOR Kinase Assay (Echo): 0.0288 μM

### EXAMPLE 43d

¹H NMR (400.13 MHz, CDCl₃) δ 1.30 (3H, d), 1.95-2.01 (3H, m), 2.14-2.26 (1H, m), 2.78-2.91 (2H, m), 3.03-3.07 (2H, m), 3.08-3.15 (2H, m), 3.25-3.32 (1H, m), 3.39 (1H, s), 3.55-3.59 (3H, m), 3.67 (3H, s), 3.68-3.71 (1H, m), 3.78 (1H, d), 3.98-4.02 (1H, m), 4.11-4.14 (1H, m), 4.42 (1H, s), 6.53 (1H, s), 7.18 (1H, s), 7.39 (2H, d), 7.44 (1H, s), 7.55 (1H, s), 7.84 (1H, s), 8.29 (2H, d).

mTOR Kinase Assay (Echo): 0.011 μM

### EXAMPLE 43e

¹H NMR (400.13 MHz, CDCl₃) δ 1.29 (3H, d), 1.92-1.99 (3H, m), 2.13-2.20 (1H, m), 2.75-2.84 (2H, m), 2.96-3.01 (2H, m), 3.05-3.12 (2H, m), 3.23-3.30 (1H, m), 3.48-3.55 (4H, m), 3.69 (1H, d), 3.77 (1H, d), 3.98 (1H, d), 4.11 (1H, d), 4.36-4.41 (3H, m), 6.51 (1H, s), 6.91 (2H, s), 7.00 (1H, t), 7.34 (2H, d), 8.23-8.25 (2H, m), 8.63 (1H, s).

mTOR Kinase Assay (Echo): 0.188 μM

### EXAMPLE 43f

¹H NMR (400.13 MHz, CDCl₃) δ 0.53-0.56 (2H, m), 0.73-0.76 (2H, m), 1.32 (3H, d), 1.63-1.66 (2H, m), 1.91-1.97 (4H, m), 2.57-2.65 (3H, m), 2.73-2.80 (3H, m), 3.05-3.11 (2H, m), 3.26-3.33 (1H, m), 3.55-3.62 (3H, m), 3.71-3.75 (1H, m), 3.81 (1H, d), 4.00-4.04 (1H, m), 4.17 (1H, d), 4.46 (1H, s), 5.81 (1H, s), 6.66 (1H, s), 7.49 (2H, d), 7.82 (1H, s), 8.31 (2H, d).

mTOR Kinase Assay (Echo): 0.0392 μM

### EXAMPLE 43g

¹H NMR (400.13 MHz, CDCl₃) δ 1.28 (3H, d), 1.58-1.64 (2H, m), 1.85-1.95 (4H, m), 2.53-2.63 (3H, m), 2.67-2.78 (3H, m), 3.12-3.16 (2H, m), 3.26-3.30 (3H, m), 3.52-3.58 (5H, m), 3.36 (1H, d), 3.77 (1H, d), 3.98 (1H, d), 4.12 (1H, d), 4.41 (1H, s), 5.05 (1H, s), 5.90 (1H, t), 6.61 (1H, s), 7.41 (2H, d), 7.96 (1H, s), 8.29 (2H, d).

mTOR Kinase Assay (Echo): 0.029 μM

### EXAMPLE 43h

¹H NMR (400.13 MHz, CDCl₃) δ 1.31 (3H, d), 1.64-1.67 (3H, m), 1.92-1.99 (5H, m), 2.47-2.64 (4H, m), 2.74-2.79 (2H, m), 3.13-3.17 (2H, m), 3.26-3.33 (1H, m), 3.43-3.48 (2H, m), 3.53-3.63 (3H, m), 3.69-3.73 (1H, m), 3.79 (1H, d), 3.99-4.03 (1H, m), 4.15 (1H, d), 4.44 (1H, s), 5.89 (1H, t), 6.66 (1H, s), 7.42 (2H, d), 7.58 (1H, s), 8.32 (2H, d).

mTOR Kinase Assay (Echo): 0.133 μM

### EXAMPLE 43i

¹H NMR (400.13 MHz, CDCl₃) δ 1.29 (3H, d), 1.54-1.62 (4H, m), 1.89-1.99 (4H, m), 2.55-2.64 (2H, m), 2.70-2.80 (2H, d), 3.12-3.19 (2H, m), 3.24-3.29 (4H, m), 3.52-3.59 (5H, m), 3.68-3.83 (3H, m), 3.98 (1H, d), 4.11-4.14 (1H, m), 4.42 (1H, s), 5.75 (1H, t), 6.62 (1H, s), 7.42 (2H, d), 7.79 (1H, s), 8.29 (2H, d).

mTOR Kinase Assay (Echo): 0.102 μM

### EXAMPLE 43j

¹H NMR (400.13 MHz, DMSO-d₆) δ 1.07 (3H, t), 1.23 (3H, d), 1.72-1.81 (2H, m), 1.88-1.96 (1H, m), 1.99-2.10 (1H, m), 2.75-2.86 (2H, m), 2.90-2.96 (2H, m), 2.98-3.04 (2H, m), 3.10-3.16 (2H, m), 3.19-3.25 (1H, m), 3.35-3.42 (2H, m), 3.46-3.55 (1H, m), 3.65 (1H, d), 3.77 (1H, d), 3.98 (1H, d), 4.24 (1H, d), 4.50-4.62 (2H, m), 6.17 (1H, s), 6.72 (1H, s), 7.49 (2H, d), 8.21 (2H, d), 8.66 (1H, s)

### EXAMPLE 43k

¹H NMR (400.13 MHz, DMSO-d₆) δ 0.89 (3H, t), 1.24 (3H, d), 1.39-1.50 (2H, m), 1.73-1.81 (2H, m), 1.86-1.96 (1H, m), 2.00-2.09 (1H, m), 2.77-2.87 (2H, m), 2.90-2.97 (2H, m), 2.97-3.08 (4H, m), 3.19-3.25 (1H, m), 3.35-3.41 (2H, m), 3.46-3.55 (1H, m), 3.65 (1H, d), 3.77 (1H, d), 3.97 (1H, d), 4.24 (1H, d), 4.53-4.60 (2H, m), 6.20 (1H, t), 6.71 (1H, s), 7.49 (2H, d), 8.22 (2H, d), 8.65 (1H, s)

### EXAMPLE 43l

¹H NMR (400.13 MHz, DMSO-d₆) δ 8.21 (2H, d), 1.23 (3H, d), 1.55-1.64 (2H, m), 1.74-1.80 (2H, m), 1.88-1.98 (1H, m), 2.01-2.10 (1H, m), 2.77-2.87 (2H, m), 2.90-2.97 (2H, m), 2.98-3.04 (2H, m), 3.13-3.24 (3H, m), 3.34-3.42 (2H, m), 3.45-3.54 (3H, m), 3.65 (1H, d), 3.77 (1H, d), 3.98 (1H, d), 4.24 (1H, d), 4.47 (1H, t), 4.53-4.59 (2H, m), 6.20 (1H, t), 6.71 (1H, s), 7.49 (2H, d), 8.71 (1H, s)

### EXAMPLE 43m

¹H NMR (400.13 MHz, DMSO-d₆) δ 1.24 (3H, d), 1.74-1.81 (2H, m), 1.88-1.97 (1H, m), 2.01-2.11 (1H, m), 2.78-2.87 (2H, m), 2.90-2.99 (2H, m), 3.00-3.06 (2H, m), 3.19-3.26 (1H, m), 3.36-3.42 (2H, m), 3.47-3.56 (1H, m), 3.66 (1H, d), 3.77 (1H, d), 3.99 (1H, d), 4.26 (1H, d), 4.53-4.61 (2H, m), 6.75 (1H, s), 6.87 (1H, s), 7.57 (2H, d), 8.30 (2H, d), 8.76 (1H, s), 9.08 (1H, s), 9.62 (1H, s)

### EXAMPLE 43n

¹H NMR (400.13 MHz, DMSO-d₆) δ 1.24 (3H, d), 1.73-1.81 (2H, m), 1.86-1.96 (1H, m), 2.01-2.10 (1H, m), 2.78-2.86 (2H, m), 2.90-2.98 (2H, m), 2.97-3.05 (2H, m), 3.13-3.26 (1H, m), 3.34-3.41 (2H, m), 3.44-3.55 (3H, m), 3.65 (1H, d), 3.77 (1H, d), 3.98 (1H, d), 4.24 (1H, d), 4.42 (1H, t), 4.51-4.59 (2H, m), 6.43 (1H, t), 6.72 (1H, s), 7.50 (2H, d), 8.23 (2H, d), 8.81 (1H, s)

### EXAMPLE 43o

¹H NMR (400.13 MHz, DMSO-d₆) δ 1.24 (3H, d), 1.72-1.81 (2H, m), 1.88-1.96 (1H, m), 2.02-2.10 (1H, m), 2.78-2.87 (2H, m), 2.91-2.98 (2H, m), 2.98-3.05 (2H, m), 3.17-3.26 (1H, m), 3.35-3.43 (2H, m), 3.47-3.60 (3H, m), 3.65 (1H, d), 3.77 (1H, d), 3.98 (1H, d), 4.24 (1H, d), 4.53-4.60 (2H, m), 5.91-6.27 (1H, m), 6.54 (1H, t), 6.73 (1H, s), 7.51 (2H, d), 8.24 (2H, d), 8.93 (1H, s)

### EXAMPLE 43p

¹H NMR (400.13 MHz, DMSO-d₆) δ 1.23 (3H, d), 1.71-1.81 (2H, m), 1.86-1.96 (1H, m), 2.02-2.11 (1H, m), 2.75-2.88 (2H, m), 2.91-2.98 (2H, m), 2.99-3.05 (2H, m), 3.17-3.26 (1H, m), 3.27-3.31 (2H, m), 3.35-3.42 (2H, m), 3.47-3.54 (1H, m), 3.65 (1H, d), 3.77 (1H, d), 3.98 (1H, d), 4.24 (1H, d), 4.51-4.60 (2H, m), 4.95 (1H, t), 6.00 (1H, s), 6.71 (1H, s), 7.45 (2H, d), 8.20 (2H, d), 8.73 (1H, s)

### EXAMPLE 43q

¹H NMR (400.13 MHz, DMSO-d₆) δ 1.08 (3H, d), 1.23 (3H, d), 1.72-1.82 (2H, m), 1.89-1.95 (1H, m), 2.02-2.09 (1H, m), 2.75-2.85 (2H, m), 2.90-2.97 (2H, m), 2.97-3.06 (2H, m), 3.17-3.25 (1H, m), 3.33-3.41 (2H, m), 3.48-3.56 (1H, m), 3.63-3.79 (4H, m), 3.98 (1H, d), 4.24 (1H, d), 4.51-4.59 (2H, m), 4.78 (1H, t), 6.10 (1H, t), 6.72 (1H, s), 7.47 (2H, d), 8.22 (2H, d), 8.71 (1H, s)

### EXAMPLE 43r

¹H NMR (400.13 MHz, DMSO-d₆) δ 1.14 (3H, d), 1.29 (3H, d), 1.78-1.88 (2H, m), 1.94-2.03 (1H, m), 2.07-2.16 (1H, m), 2.81-2.93 (2H, m), 2.96-3.03 (2H, m), 3.03-3.10 (2H, m), 3.23-3.32 (1H, m), 3.38-3.48 (3H, m), 3.53-3.61 (1H, m), 3.68-3.84 (4H, m), 4.03 (1H, d), 4.30 (1H, d), 4.58-4.65 (2H, m), 4.83 (1H, t), 6.15 (1H, t), 6.77 (1H, s), 7.53 (2H, d), 8.27 (2H, d), 8.77 (1H, s)

### EXAMPLE 43s

¹H NMR (400.13 MHz, DMSO-d₆) δ 1.25 (3H, d), 1.74-1.82 (2H, m), 1.89-1.98 (1H, m), 2.03-2.10 (1H, m), 2.77-2.90 (2H, m), 2.92-3.00 (2H, m), 3.02-3.07 (2H, m), 3.20-3.26 (1H, m), 3.36-3.44 (2H, m), 3.46-3.56 (1H, m), 3.66 (1H, d), 3.78 (1H, d), 3.99 (1H, d), 4.27 (1H, d), 4.51-4.63 (2H, m), 6.77 (1H, s), 7.63 (2H, d), 8.28-8.39 (3H, m), 9.46 (1H, s)

### EXAMPLE 43t

¹H NMR (400.13 MHz, DMSO-d₆) δ 1.25 (3H, d), 1.74-1.83 (2H, m), 1.88-1.96 (1H, m), 2.00-2.09 (1H, m), 2.77-2.88 (2H, m), 2.91-2.98 (2H, m), 3.00-3.07 (2H, m), 3.20-3.25 (1H, m), 3.37-3.43 (2H, m), 3.46-3.56 (1H, m), 3.66 (1H, d), 3.78 (1H, d), 3.99 (1H, d), 4.26 (1H, d), 4.53-4.62 (2H, m), 6.76 (1H, s), 7.14 (1H, s), 7.40 (1H, s), 7.59 (2H, d), 8.31 (2H, d), 9.20 (1H, s)

The preparation of phenyl N-[4-[4-[1-(3-hydroxypropylsulfonyl)cyclobutyl]-6-[(3S)-3-methylmorpholin-4-yl]pyrimidin-2-yl]phenyl]carbamate is described below:

### Phenyl N-[4-[4-[1-(3-hydroxypropylsulfonyl)cyclobutyl]-6-[(3S)-3-methylmorpholin-4-yl]pyrimidin-2-yl]phenyl]carbamate

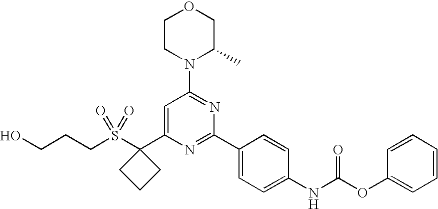

Phenyl chloroformate (0.632 mL, 5.04 mmol) was added dropwise to 3-[1-[2-(4-aminophenyl)-6-[(3S)-3-methylmorpholin-4-yl]pyrimidin-4-yl]cyclobutyl]sulfonylpropan-1-ol (1.5 g, 3.36 mmol) and sodium hydrogen carbonate (0.423 g, 5.04 mmol) in dioxane (33.6 mL), cooled to 10° C. under a nitrogen atmosphere. The resulting mixture was stirred at RT for 2 hours. The reaction mixture was diluted with ethyl acetate (300 mL), and washed with water (150 mL). The organic layer was dried (Na₂SO₄), filtered and evaporated to afford crude product as a yellow gum. This material was used directly in the next step without further purification.

LCMS Spectrum: m/z (ESI+) (M+H)+=565; HPLC tR=2.71 min.

### 3-[1-[2-(4-Aminophenyl)-6-[(3S)-3-methylmorpholin-4-yl]pyrimidin-4-yl]cyclobutyl]sulfonylpropan-1-ol

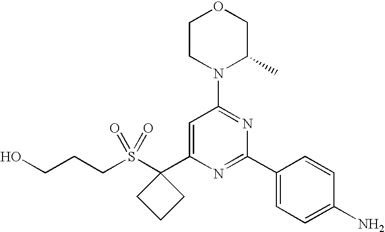

A solution of tetrabutylammonium fluoride (18.25 mL, 18.25 mmol) in THF was added to a stirred solution of 4-[4-[(3S)-3-methylmorpholin-4-yl]-6-[1-[3-tri(propan-2-yl)silyloxypropylsulfonyl]cyclobutyl]pyrimidin-2-yl]aniline (2.2 g, 3.65 mmol) in THF (24.33 mL) at RT. The resulting solution was stirred at RT for 2 hours. The reaction mixture was concentrated and diluted with ethyl acetate (150 mL), and washed with water (100 mL). The organic layer was dried (Na₂SO₄), filtered and evaporated to afford crude product. The crude product was purified by flash silica chromatography, elution gradient 0 to 100% ethyl acetate in isohexane, to give the desired material as a pale yellow oil which solidified on standing (1.50 g).

NMR Spectrum: ¹H NMR (400.13 MHz, CDCl₃) δ 1.33 (3H, d), 1.57 (1H, t), 1.94-2.04 (3H, m), 2.18-2.25 (1H, m), 2.82-2.88 (2H, m), 2.98 (2H, t), 3.09-3.16 (2H, m), 3.28-3.35 (1H, m), 3.63 (3H, q), 3.73-3.76 (1H, m), 3.82 (1H, d), 3.90 (2H, s), 4.01-4.05 (1H, m), 4.16 (1H, d), 4.47 (1H, d), 6.51 (1H, s), 6.70-6.72 (2H, m), 8.22-8.24 (2H, m).

LCMS Spectrum: m/z (ESI+) (M+H)+=447; HPLC tR=2.09 min.

### 4-[4-[(3S)-3-Methylmorpholin-4-yl]-6-[1-[3-tri(propan-2-yl)silyloxypropylsulfonyl]cyclobutyl]pyrimidin-2-yl]aniline

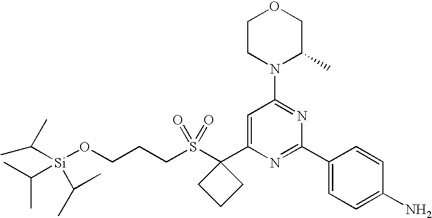

Dichlorobis(triphenylphosphine)palladium(II) (0.132 g, 0.19 mmol) was added in one portion to a carefully degassed solution of 3-[1-[2-chloro-6-[(3S)-3-methylmorpholin-4-yl]pyrimidin-4-yl]cyclobutyl]sulfonylpropoxy-tri(propan-2-yl)silane (2.05 g, 3.75 mmol), 4-(4,4,5,5-tetramethyl-1,3,2-dioxaborolan-2-yl)aniline (1.233 g, 5.63 mmol) and 2M aqueous sodium carbonate solution (6.57 mL, 13.14 mmol) in a solvent mixture of DMF (6.82 mL), water (17.06 mL), ethanol (6.82 mL) and DME (6.82 mL). The resulting mixture was stirred at 80° C. for 4 hours. The cooled reaction mixture was diluted with ethyl acetate (200 mL), and washed sequentially with water (100 mL) and saturated brine (100 mL). The organic layer was dried (Na₂SO₄), filtered and evaporated to afford crude product. The crude product was purified by flash silica chromatography, elution gradient 0 to 75% ethyl acetate in isohexane, to give the desired material as a pale yellow foam which solidified under vacuum (2.2 g).

NMR Spectrum: ¹H NMR (400.13 MHz, CDCl₃) δ 0.89-0.98 (21H, m), 1.32 (3H, s), 1.92-1.99 (3H, m), 2.15-2.25 (1H, m), 2.84-2.87 (2H, m), 2.91-2.95 (2H, m), 3.07-3.13 (2H, m), 3.26-3.34 (1H, m), 3.57-3.62 (1H, m), 3.65 (2H, t), 3.74 (1H, dd), 3.81 (1H, d), 3.88 (2H, s), 4.03 (1H, dd), 4.15 (1H, d), 4.47 (1H, s), 6.52 (1H, s), 6.68-6.70 (2H, m), 8.21-8.23 (2H, m).

LCMS Spectrum: m/z (ESI+) (M+H)+=603; HPLC tR=3.82 min.

### 3-[1-[2-Chloro-6-[(3S)-3-methylmorpholin-4-yl]pyrimidin-4-yl]cyclobutyl]sulfonylpropoxy-tri(propan-2-yl)silane

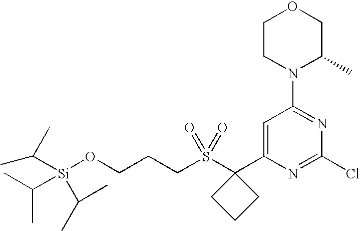

Aqueous sodium hydroxide solution (50% w/w, 48.7 mL) was added to 3-[[2-chloro-6-[(3S)-3-methylmorpholin-4-yl]pyrimidin-4-yl]methylsulfonyl]propoxy-tri(propan-2-yl)silane (5.6 g, 11.06 mmol), 1,3-dibromopropane (3.37 mL, 33.19 mmol) and tetrabutylammonium bromide (0.357 g, 1.11 mmol) in toluene (221 mL) at RT. The resulting suspension was stirred at 45° C. for 1 hour. Water was added to the solution. The toluene was washed with water twice, dried (MgSO₄), filtered and evaporated. The crude product was purified by flash silica chromatography, elution gradient 0 to 40% ethyl acetate in DCM, to give the desired material as a colourless gum (2.05 g).

NMR Spectrum: ¹H NMR (400.13 MHz, CDCl₃) δ 0.97-1.08 (21H, m), 1.32 (3H, d), 1.94-2.01 (3H, m), 2.21-2.23 (1H, m), 2.70-2.76 (2H, m), 2.93 (2H, q), 3.03-3.08 (2H, m), 3.28-3.32 (1H, m), 3.51-3.57 (1H, m), 3.66-3.70 (1H, m), 3.76 (3H, t), 3.98-4.02 (2H, m), 4.32 (1H, s), 6.55 (1H, s).

LCMS Spectrum: m/z (ESI+) (M+H)+=546; HPLC tR=4.05 min.

The preparation of 3-[[2-chloro-6-[(3S)-3-methylmorpholin-4-yl]pyrimidin-4-yl]methylsulfonyl]propoxy-tri(propan-2-yl)silane was described earlier.

The preparation of phenyl N-[4-[4-[1-(3-hydroxypropylsulfonyl)cyclopentyl]-6-[(3S)-3-methylmorpholin-4-yl]pyrimidin-2-yl]phenyl]carbamate is described below:

### Phenyl N-[4-[4-[1-(3-hydroxypropylsulfonyl)cyclopentyl]-6-[(3S)-3-methylmorpholin-4-yl]pyrimidin-2-yl]phenyl]carbamate

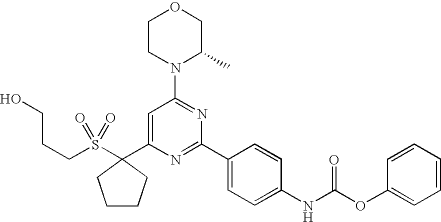

Phenyl chloroformate (1.512 mL, 12.05 mmol) was added dropwise to 3-[1-[2-(4-aminophenyl)-6-[(3S)-3-methylmorpholin-4-yl]pyrimidin-4-yl]cyclopentyl]sulfonylpropan-1-ol (3.7 g, 8.03 mmol) and sodium hydrogen carbonate (1.012 g, 12.05 mmol) in dioxane (80 mL) cooled to 10° C. under a nitrogen atmosphere. The resulting mixture was stirred at RT for 2 hours. The reaction mixture was diluted with ethyl acetate (300 mL), and washed with water (150 mL). The organic layer was dried (Na₂SO₄), filtered and evaporated to afford crude product. The crude solid was triturated with a mixture of diethyl ether and isohexane to give the desired material as a yellow solid which was used without further purification (3.60 g).

LCMS Spectrum: m/z (ESI+) (M+H)+=581; HPLC tR=2.83 min.

### 3-[1-[2-(4-Aminophenyl)-6-[(3S)-3-methylmorpholin-4-yl]pyrimidin-4-yl]cyclopentyl]sulfonylpropan-1-ol

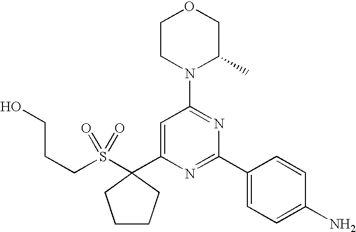

A solution of tetrabutylammonium fluoride (46.2 mL, 46.20 mmol) in THF was added to a stirred solution of 4-[4-[(3S)-3-methylmorpholin-4-yl]-6-[1-[3-tri(propan-2-yl)silyloxypropylsulfonyl]cyclopentyl]pyrimidin-2-yl]aniline (5.7 g, 9.24 mmol) in THF (61.6 mL) at RT. The resulting solution was stirred at RT for 2 hours. The reaction mixture was concentrated and diluted with ethyl acetate (250 mL), and washed with water (150 mL). The organic layer was dried (Na₂SO₄), filtered and evaporated to afford crude product. The crude product was purified by flash silica chromatography, elution gradient 0 to 100% ethyl acetate in isohexane, to give the desired material as a pale yellow oil which solidified on standing (3.70 g).

NMR Spectrum: ¹H NMR (400.13 MHz, CDCl₃) δ 1.32 (3H, d), 1.59 (1H, t), 1.61-1.70 (2H, m), 1.92-1.98 (4H, m), 2.57-2.66 (2H, m), 2.74-2.83 (2H, m), 3.07 (2H, t), 3.27-3.34 (1H, m), 3.57-3.65 (3H, m), 3.73-3.77 (1H, m), 3.82 (1H, d), 3.90 (2H, s), 4.01-4.05 (1H, m), 4.11-4.18 (1H, m), 4.46 (1H, d), 6.63 (1H, s), 6.71 (2H, d), 8.23 (2H, d).

LCMS Spectrum: m/z (ESI+) (M+H)+=461; HPLC tR=2.18 min.

### 4-[4-[(3S)-3-Methylmorpholin-4-yl]-6-[1-[3-tri(propan-2-yl)silyloxypropylsulfonyl]cyclopentyl]pyrimidin-2-yl]aniline

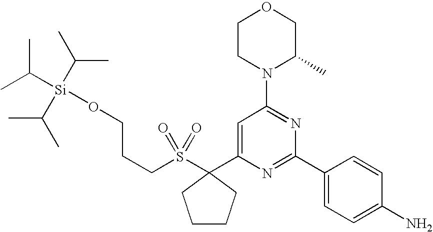

Dichlorobis(triphenylphosphine)palladium(II) (0.345 g, 0.49 mmol) was added in one portion to a carefully degassed solution of 3-[1-[2-chloro-6-[(3S)-3-methylmorpholin-4-yl]pyrimidin-4-yl]cyclopentyl]sulfonylpropoxy-tri(propan-2-yl)silane (5.5 g, 9.82 mmol), 4-(4,4,5,5-tetramethyl-1,3,2-dioxaborolan-2-yl)aniline (3.23 g, 14.73 mmol) and 2M aqueous sodium carbonate solution (17.18 mL, 34.36 mmol) in a solvent mixture of DMF (17.85 mL), water (44.6 mL), ethanol (17.85 mL) and DME (17.85 mL). The resulting mixture was stirred at 80° C. for 4 hours. The cooled reaction mixture was diluted with ethyl acetate (200 mL), and washed sequentially with water (100 mL) and saturated brine (100 mL). The organic layer was dried (MgSO₄), filtered and evaporated to afford crude product. The crude product was purified by flash silica chromatography, elution gradient 0 to 75% ethyl acetate in DCM, to give the desired material as a pale yellow foam which solidified under vacuum (5.70 g).

NMR Spectrum: ¹H NMR (400.13 MHz, CDCl₃) δ 0.90-0.98 (21H, m), 1.32 (4H, t), 1.63-1.66 (2H, m), 1.89-1.96 (4H, m), 2.59-2.64 (2H, m), 2.77-2.81 (2H, m), 2.98-3.02 (2H, m), 3.29-3.32 (1H, m), 3.57-3.63 (1H, m), 3.66 (2H, t), 3.72-3.76 (1H, m), 3.81 (1H, d), 3.88 (2H, s), 4.01-4.05 (1H, m), 4.15 (1H, d), 4.45 (1H, s), 6.66 (1H, s), 6.69 (2H, d), 8.22 (2H, d).

LCMS Spectrum: m/z (ESI+) (M+H)+=617; HPLC tR=3.91 min.

### 3-[1-[2-Chloro-6-[(3S)-3-methylmorpholin-4-yl]pyrimidin-4-yl]cyclopentyl]sulfonylpropoxy-tri(propan-2-yl)silane

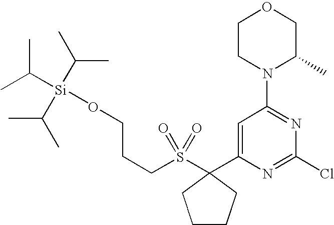

Aqueous sodium hydroxide solution (50% w/w aq, 48.7 mL) was added to 3-[[2-chloro-6-[(3S)-3-methylmorpholin-4-yl]pyrimidin-4-yl]methylsulfonyl]propoxy-tri(propan-2-yl)silane (5.6 g, 11.06 mmol), 1,4-dibromobutane (3.93 mL, 33.19 mmol) and tetrabutylammonium bromide (0.357 g, 1.11 mmol) in toluene (221 mL) at RT. The resulting suspension was stirred at 45° C. for 1 hour and the toluene washed with water twice, dried (MgSO₄), filtered and evaporated. The crude product was purified by flash silica chromatography, elution gradient 0 to 40% ethyl acetate in DCM, to give the desired material as a white solid (5.57 g).

NMR Spectrum: ¹H NMR (400.13 MHz, CDCl₃) δ 0.98-1.08 (21H, m), 1.31 (3H, d), 1.60-1.65 (2H, m), 1.88-1.99 (4H, m), 2.56-2.61 (4H, m), 2.97-3.00 (2H, m), 3.28-3.32 (1H, m), 3.51-3.58 (1H, m), 3.67-3.71 (1H, m), 3.76 (3H, t), 3.98-4.02 (2H, m), 4.31, (1H, s), 6.71 (1H, s).

LCMS Spectrum: m/z (ESI+) (M+H)+=560; HPLC tR=3.86 min.

The preparation of 3-[[2-chloro-6-[(3S)-3-methylmorpholin-4-yl]pyrimidin-4-yl]methylsulfonyl]propoxy-tri(propan-2-yl)silane was described earlier.

### EXAMPLE 44

### N-[2-[1-[2-[4-(Cyclopropylcarbamoylamino)phenyl]-6-[(3S)-3-methylmorpholin-4-yl]pyrimidin-4-yl]cyclopropyl]sulfonylethyl]acetamide

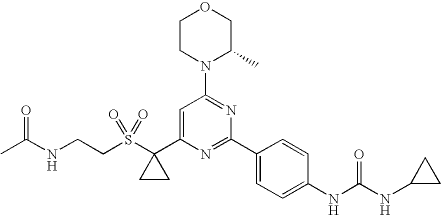

Cyclopropylamine (0.120 mL, 1.73 mmol) was added in one portion to a stirred solution of phenyl N-[4-[4-[1-(2-acetamidoethylsulfonyl)cyclopropyl]-6-[(3S)-3-methylmorpholin-4-yl]pyrimidin-2-yl]phenyl]carbamate (0.2 g, 0.35 mmol) in THF (34.5 mL) at RT. The resulting solution was stirred at 40° C. for 24 hours. The reaction mixture was then concentrated, and the crude product was purified by flash silica chromatography, elution gradient 0 to 5% methanol in DCM, to give the desired material as a white solid (0.135 g).

NMR Spectrum: ¹H NMR (400.13 MHz, CDCl₃) δ 0.66-0.70 (2H, m), 0.84-0.89 (2H, m), 1.34 (3H, d), 1.49-1.58 (2H, m), 1.82-1.85 (2H, m), 1.93 (3H, s), 2.61-2.66 (1H, m), 3.29-3.36 (1H, m), 3.48-3.51 (2H, m), 3.56-3.63 (1H, m), 3.72-3.75 (1H, m), 3.79-3.84 (3H, m), 4.03-4.06 (1H, m), 4.15 (1H, d), 4.48 (1H, s), 5.05 (1H, s), 6.67 (1H, s), 6.74 (1H, d), 7.13 (1H, s), 7.51 (2H, d), 8.26 (2H, d).

LCMS Spectrum: m/z (ESI+) (M+H)+=543; HPLC tR=1.86 min.

mTOR Kinase Assay (Echo): 0.0234 μM

The preparation of phenyl N-[4-[4-[1-(2-acetamidoethylsulfonyl)cyclopropyl]-6-[(3S)-3-methylmorpholin-4-yl]pyrimidin-2-yl]phenyl]carbamate is described below:

### Phenyl N-[4-[4-[1-(2-acetamidoethylsulfonyl)cyclopropyl]-6-[(3S)-3-methylmorpholin-4-yl]pyrimidin-2-yl]phenyl]carbamate

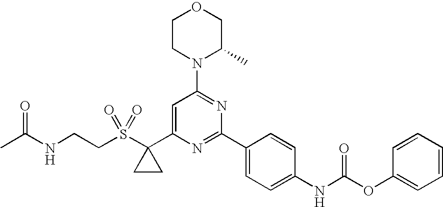

Phenyl chloroformate (0.328 mL, 2.61 mmol) was added dropwise to N-[2-[1-[2-(4-aminophenyl)-6-[(3S)-3-methylmorpholin-4-yl]pyrimidin-4-yl]cyclopropyl]sulfonylethyl]acetamide (800 mg, 1.74 mmol) and sodium hydrogen carbonate (219 mg, 2.61 mmol) in dioxane (174 mL) cooled to 10° C. under a nitrogen atmosphere. The resulting mixture was stirred at RT for 2 hours. The reaction mixture was diluted with ethyl acetate (300 mL), and washed with water (150 mL). The organic layer was dried (Na₂SO₄), filtered and evaporated to afford crude product. The crude solid was triturated with a mixture of diethyl ether and isohexane to give the desired material as a yellow solid (741 mg). This material was used directly without further purification.

LCMS Spectrum: m/z (ESI+) (M+H)+=580; HPLC tR=2.44 min.

### N-[2-[1-[2-(4-Aminophenyl)-6-[(3S)-3-methylmorpholin-4-yl]pyrimidin-4-yl]cyclopropyl]sulfonylethyl]acetamide

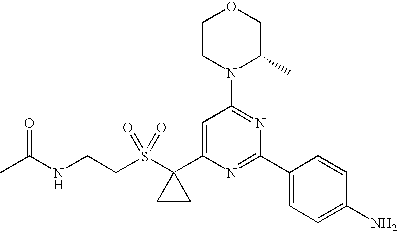

Dichlorobis(triphenylphosphine)palladium(II) (0.174 g, 0.25 mmol) was added in one portion to a carefully degassed solution of N-[2-[1-[2-chloro-6-[(3S)-3-methylmorpholin-4-yl]pyrimidin-4-yl]cyclopropyl]sulfonylethyl]acetamide (2 g, 4.96 mmol), 4-(4,4,5,5-tetramethyl-1,3,2-dioxaborolan-2-yl)aniline (1.414 g, 6.45 mmol) and 2M aqueous sodium carbonate solution (8.69 mL, 17.37 mmol) in a solvent mixture of DMF (9.03 mL), water (22.56 mL), ethanol (9.03 mL) and DME (9.03 mL). The resulting mixture was stirred at 80° C. for 4 hours. The cooled reaction mixture was diluted with ethyl acetate (200 mL), and washed sequentially with water (100 mL) and saturated brine (100 mL). The organic layer was dried (MgSO₄), filtered and evaporated to afford crude product. The crude product was purified by flash silica chromatography, elution gradient 0 to 75% ethyl acetate in DCM, to give the desired material as a pale yellow foam which solidified under vacuum (1.805 g).

NMR Spectrum: ¹H NMR (400.13 MHz, CDCl₃) δ 1.33 (3H, d), 1.49-1.51 (2H, m), 1.80-1.84 (2H, m), 1.93 (3H, s), 3.28-3.35 (1H, m), 3.47-3.50 (2H, m), 3.55-3.62 (1H, m), 3.71-3.75 (1H, m), 3.79-3.83 (3H, m), 3.92 (2H, s), 4.01-4.05 (1H, m), 4.10-4.17 (1H, m), 4.47 (1H, s), 6.61 (1H, s), 6.69-6.72 (2H, m), 6.81 (1H, s), 8.13-8.16 (2H, m).

LCMS Spectrum: m/z (ESI+) (M+H)+=460; HPLC tR=1.79 min.

### N-[2-[1-[2-Chloro-6-[(3S)-3-methylmorpholin-4-yl]pyrimidin-4-yl]cyclopropyl]sulfonylethyl]acetamide

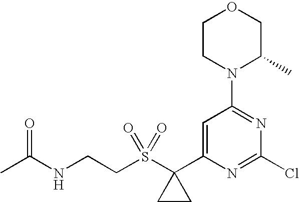

Aqueous sodium hydroxide solution (50% aq, 8.52 mL) was added to N-[2-[[2-chloro-6-[(3S)-3-methylmorpholin-4-yl]pyrimidin-4-yl]methylsulfonyl]ethyl]acetamide (3.21 g, 8.52 mmol), 1,2-dibromoethane (1.468 mL, 17.03 mmol) and tetrabutylammonium bromide (0.549 g, 1.70 mmol) in toluene (122 mL) at RT. The resulting solution was stirred at 60° C. for 3 hours. The reaction mixture was evaporated to dryness and redissolved in ethyl acetate (200 mL), and washed sequentially with water (200 mL) and saturated brine (100 mL). The organic layer was dried (MgSO₄), filtered and evaporated to afford crude product. The crude product was purified by flash silica chromatography, elution gradient 0 to 50% ethyl acetate in isohexane, to give the desired material as a yellow solid (2.04 g).

NMR Spectrum: ¹H NMR (400.13 MHz, CDCl₃) δ 1.34 (3H, d), 1.44-1.47 (2H, m), 1.80-1.84 (2H, m), 2.02 (3H, s), 3.27-3.34 (1H, m), 3.35-3.38 (2H, m), 3.51-3.57 (1H, m), 3.66-3.70 (1H, m), 3.75-3.80 (3H, m), 3.99-4.03 (2H, m), 4.34 (1H, s), 6.73 (1H, s), 6.88 (1H, s).

LCMS Spectrum: m/z (ESI+) (M+H)+=403; HPLC tR=1.51 min.

### N-[2-[[2-Chloro-6-[(3S)-3-methylmorpholin-4-yl]pyrimidin-4-yl]methylsulfonyl]ethyl]acetamide

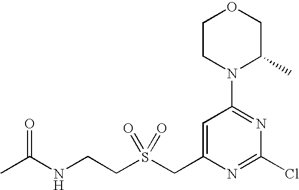

N-[2-[[2-Chloro-6-[(3S)-3-methylmorpholin-4-yl]pyrimidin-4-yl]methylsulfanyl]ethyl]acetamide (3.24 g, 9.40 mmol) was dissolved in dioxane (28.2 mL) and 2N sulfuric acid (0.282 mL) was added. The solution was heated to 55° C. Sodium tungstate dihydrate (0.062 g, 0.19 mmol) dissolved in water (2.82 mL) was added to the solution and allowed to stir for 5 minutes. Hydrogen peroxide (5.42 mL, 56.37 mmol) was then added dropwise over several minutes. The solution was heated at 55° C. for 2.5 hours. The heat was removed and water (300 mL) was added. The resulting suspension was stirred for 30 minutes. The solids were filtered, rinsed with water and dried in a vacuum oven at 50° C. overnight, to give the desired material as a white solid (3.30 g).

NMR Spectrum: ¹H NMR (400.13 MHz, DMSO-d₆) δ 1.22 (3H, d), 1.82 (3H, s), 3.19-3.26 (1H, m), 3.36-3.43 (2H, m), 3.45-3.51 (3H, m), 3.58-3.62 (1H, m), 3.73 (1H, d), 3.92-3.96 (2H, m), 4.30 (1H, s), 4.49 (2H, s), 6.92 (1H, s), 8.11 (1H, t).

LCMS Spectrum: m/z (ESI+) (M+H)+=377; HPLC tR=1.37 min.

### N-2-[[2-Chloro-6-[(3S)-3-methylmorpholin-4-yl]pyrimidin-4-yl]methylsulfanyl]ethyl]acetamide

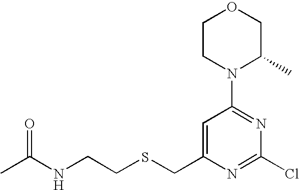

N-Acetylcysteamine (1.804 mL, 16.97 mmol) was added to 2-chloro-4-(iodomethyl)-6-[(3S)-3-methylmorpholin-4-yl]pyrimidine (4 g, 11.31 mmol) and DIPEA (4.93 mL, 28.28 mmol) in acetonitrile (226 mL) at RT. The resulting solution was stirred at RT for 3 hours. The solvent was removed in vacuo, and the crude material was then purified by flash silica chromatography, eluting with ethyl acetate, to give the desired material as a white solid (3.24 g).

NMR Spectrum: ¹H NMR (400.13 MHz, CDCl₃) δ 1.33 (3H, d), 2.01 (3H, s), 2.71 (2H, t), 3.25-3.33 (1H, m), 3.48-3.58 (3H, m), 3.59 (2H, s), 3.67-3.71 (1H, m), 3.79 (1H, d), 3.99-4.03 (2H, m), 4.33 (1H, s), 6.40 (1H, s), 6.55 (1H, s).

LCMS Spectrum: m/z (ESI+) (M+H)+=345; HPLC tR=1.54 min.

The preparation of 2-chloro-4-(iodomethyl)-6-[(3S)-3-methylmorpholin-4-yl]pyrimidine was described earlier.

### EXAMPLE 45

### N-[2-[1-[2-[4-(Ethylcarbamoylamino)phenyl]-6-[(3S)-3-methylmorpholin-4-yl]pyrimidin-4-yl]cyclopropyl]sulfonylethyl]acetamide

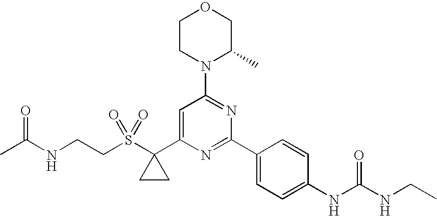

A solution of ethylamine (2M in THF, 0.863 mL, 1.73 mmol) was added in one portion to a stirred solution of phenyl N-[4-[4-[1-(2-acetamidoethylsulfonyl)cyclopropyl]-6-[(3S)-3-methylmorpholin-4-yl]pyrimidin-2-yl]phenyl]carbamate (0.2 g, 0.35 mmol) in THF (34.5 mL) at RT. The resulting solution was stirred at 50° C. for 2 hours. The product was concentrated in vacuo, then purified by flash silica chromatography, elution gradient 0 to 10% methanol in DCM, to give a residue that was triturated with acetonitrile to give the desired material as a white solid (0.147 g).

NMR Spectrum: ¹H NMR (400.13 MHz, CDCl3) δ 1.16 (3H, t), 1.33 (3H, d), 1.48-1.51 (2H, m), 1.81-1.84 (2H, m), 1.93 (3H, s), 3.27-3.34 (3H, m), 3.49-3.52 (2H, m), 3.55-3.60 (1H, m), 3.70-3.74 (1H, m), 3.78-3.83 (3H, m), 4.01-4.05 (1H, m), 4.10-4.16 (1H, m), 4.46 (1H, s), 5.18 (1H, t), 6.64 (1H, s), 6.89 (1H, t), 7.15 (1H, s), 7.42 (2H, d), 8.23 (2H, d).

LCMS Spectrum: m/z (ESI+) (M+H)+=531; HPLC tR=1.87 min.

mTOR Kinase Assay (Echo): 0.0219 μM

The preparation of phenyl N-[4-[4-[1-(2-acetamidoethylsulfonyl)cyclopropyl]-6-[(3S)-3-methylmorpholin-4-yl]pyrimidin-2-yl]phenyl]carbamate was described earlier.

### EXAMPLE 46

### 2-[1-[2-[4-(Cyclopropylcarbamoylamino)phenyl]-6-morpholin-4-ylpyrimidin-4-yl]cyclopropyl]sulfonylacetamide

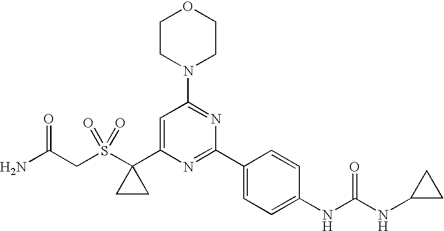

Bis(triphenylphosphine)palladium(II) chloride (19.03 mg, 0.03 mmol) was added to 2-[1-(2-chloro-6-morpholin-4-ylpyrimidin-4-yl)cyclopropyl]sulfonylacetamide (146 mg, 0.40 mmol), 1-cyclopropyl-3-(4-(4,4,5,5-tetramethyl-1,3-dioxolan-2-yl)phenyl)urea (185 mg, 0.61 mmol) and sodium carbonate (0.809 mL, 1.62 mmol) in a solvent mixture of DMF (3 mL), DME (8 mL), water (2 mL) and ethanol (1.5 mL) at RT. The resulting mixture was stirred at 90° C. for 18 hours under an inert atmosphere. The reaction mixture was diluted with ethyl acetate (100 mL), and washed with water (100 mL followed by 75 mL). The organic layer was dried (Na₂SO₄), filtered and evaporated to afford crude product. The crude gum was triturated with a mixture of methanol and DCM and the solid removed by filtration. The filtrate was purified by flash silica chromatography, elution gradient 0 to 7% methanol in ethyl acetate, to give a gum which was further purified by ion exchange chromatography on an SCX column, eluting with 7N ammonia in methanol, to give a beige solid which was further purified by prep HPLC to give the desired material (7 mg).

LCMS Spectrum: m/z (ESI+)(M+H)+=499; HPLC tR=1.67 min.

mTOR Kinase Assay (Echo): 0.00456 μM

The following compound was prepared in an analogous fashion from 2-[1-(2-chloro-6-morpholin-4-ylpyrimidin-4-yl)cyclopropyl]sulfonyl-N-methylacetamide.

| | | | LCMS | Retention |
|---|---|---|---|---|
| Example | Structure | NAME | MH+ | time (min) |
| 46a | 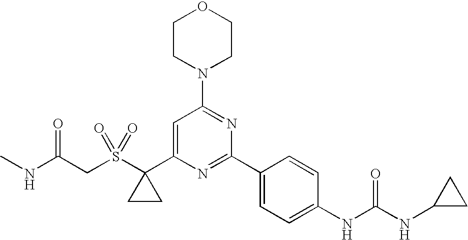 | 2-[1-[2-[4-(cyclopropylcarbamoylamino)phe-nyl]-6-morpholin-4-ylpyrimidin-4-yl]cyclopropyl]sulfonyl-N-methylacetamide | 515 | 1.74 |

### EXAMPLE 46a

¹H NMR (400.132 MHz, DMSO-d₆) δ 0.39-0.43 (2H, m), 0.61-0.67 (2H, m), 1.57-1.61 (2H, m), 1.66-1.71 (2H, m), 2.63 (3H, d), 3.72 (8H, s), 4.39 (2H, s), 6.46 (1H, s), 6.87 (1H, s), 7.51 (2H, d), 8.20 (2H, d), 8.29 (1H, d), 8.58 (1H, s).

mTOR Kinase Assay (Echo): 0.0126 μM

The preparation of 2-[1-(2-chloro-6-morpholin-4-ylpyrimidin-4-yl)cyclopropyl]sulfonyl-N-methylacetamide is described below.

### 2-[1-(2-Chloro-6-mopholin-4-ylpyrimidin-4-ylcyclopropyl]sulfonyl-N-methylacetamide

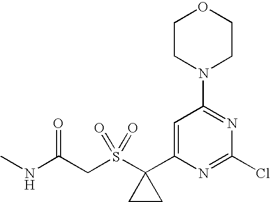

HATU (252 mg, 0.66 mmol) was added to 2-[1-(2-chloro-6-morpholin-4-ylpyrimidin-4-yl)cyclopropyl]sulfonylacetic acid (200 mg, 0.55 mmol) and triethylamine (0.077 mL, 0.55 mmol) in DCM (10 mL) at RT under a nitrogen atmosphere and stirred for 15 minutes. Methylamine (2M in THF, 2 mL) was added and reaction stirred for 18 hours. The reaction mixture was diluted with ethyl acetate (100 mL), and washed with a saturated solution of sodium hydrogen carbonate (100 mL). The organic layer was dried (Na₂SO₄), filtered and evaporated to afford crude product. The crude product was purified by flash silica chromatography, elution gradient 50 to 90% ethyl acetate in isohexane, to give the desired material as a yellow gum (156 mg)

NMR Spectrum: ¹H NMR (400.132 MHz, DMSO-d₆) δ 1.51-1.54 (2H, m), 1.62-1.67 (2H, m), 2.62 (3H, d), 3.66 (8H, m), 4.27 (2H, s), 7.06 (1H, s), 8.22 (1H, s)

LCMS Spectrum: m/z (ESI+)(M+H)+=375; HPLC tR=1.35 min.

2-[1-(2-Chloro-6-morpholin-4-ylpyrimidin-4-yl)cyclopropyl]sulfonylacetamide was made by an analogous procedure to that above (chromatographed using 0-4% methanol in ethyl acetate).

| | | LCMS | Retention |
|---|---|---|---|
| Structure | NAME | MH+ | time (min) |
| 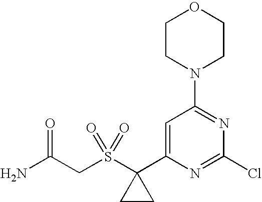 | 2-[1-(2-chloro-6-morpholin-4-ylpyrimidin-4-yl)cyclopropyl]sulfonylacetamide | 361 | 1.31 |

The preparation of 2-[1-(²-chloro-6-morpholin-4-ylpyrimidin-4-yl)cyclopropyl]sulfonylacetic acid is described below.

### 2-[1-(2-Chloro-6-morpholin-4-yl)cyclopropyl]sulfonylacetic acid

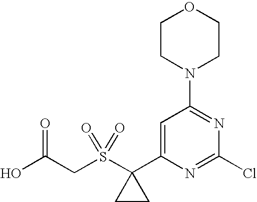

Lithium hydroxide (0.812 g, 33.93 mmol) was added to methyl 2-[1-(2-chloro-6-morpholin-4-ylpyrimidin-4-yl)cyclopropyl]sulfonylacetate (2.55 g, 6.79 mmol), in a mixture of THF (40 mL) and water (8 mL). The resulting mixture was stirred at RT for 2 hours then acidified with 2M hydrochloric acid. The reaction mixture was extracted with ethyl acetate (400 mL) and the organic layer dried (Na₂SO₄), filtered and evaporated to afford crude product. The crude solid was triturated with a mixture of diethyl ether and isohexane to give the desired material as a white solid (1.96 g).

NMR Spectrum: ¹H NMR (400.132 MHz, DMSO-d₆) δ 1.53-1.58 (2H, m), 1.69-1.72 (2H, m), 3.66 (8H, s), 4.55 (2H, s), 6.98 (1H, s)

LCMS Spectrum: m/z (ESI+)(M+H)+=362; HPLC tR=0.69 min.

### Methyl 2-[1-(2-chloro-6-morpholin-4-ylpyrimidin-4-yl)cyclopropyl]sulfonylacetate

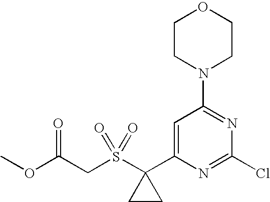

Lithium diisopropylamide (8.92 mL, 16.05 mmol) was added dropwise to 2-chloro-4-(1-methylsulfonylcyclopropyl)-6-morpholin-4-ylpyrimidine (4.25 g, 13.37 mmol), in THF (80 mL) at −78° C. under a nitrogen atmosphere. The resulting mixture was stirred at −78° C. for 15 minutes. Dimethyl carbonate (5.63 mL, 66.87 mmol) was added and the resulting mixture stirred at −78° C. for 10 minutes, then left to warm to RT. The mixture was cooled back to −78° C. and additional lithium diisopropylamide (8.92 mL, 16.05 mmol) added. The mixture was stirred at −78° C. for 10 minutes, then dimethyl carbonate (5.63 mL, 66.87 mmol) added and the mixture allowed to come to RT and the pH adjusted to 7 with 2M hydrochloric acid. The reaction mixture was diluted with ethyl acetate (350 mL), and washed with water (150 mL). The organic layer was dried (Na₂SO₄), filtered and evaporated to afford crude product. The crude product was purified by flash silica chromatography, elution gradient 10 to 70% ethyl acetate in isohexane, to give a yellow solid which was triturated with a mixture of diethyl ether and isohexane to give the desired material as a white solid (3.10 g).

NMR Spectrum: ¹H NMR (400.132 MHz, DMSO-d₆) δ 1.56-1.60 (2H, m), 1.68-1.72 (2H, m), 3.63-3.70 (1 1H, m), 4.71 (2H, s), 6.97 (1H, s)

LCMS Spectrum: m/z (ESI+)(M+H)+=376; HPLC tR=1.76 min.

The preparation of 2-chloro-4-(1-methylsulfonylcyclopropyl)-6-morpholin-4-ylpyrimidine was described earlier.

### EXAMPLE 47

### 3-Cyclopropyl-1-[4-[4-[1-(2-hydroxyethylsulfonyl)cyclopropyl]-6-morpholin-4-ylpyrimidin-2-yl]phenyl]urea

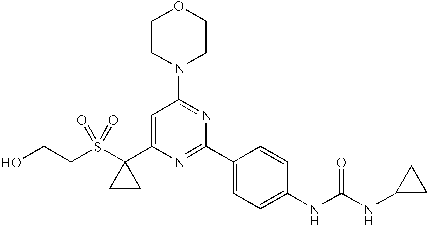

A solution of phenyl N-[4-[4-[1-(2-hydroxyethylsulfonyl)cyclopropyl]-6-morpholin-4-ylpyrimidin-2-yl]phenyl]carbamate (50 mg, 0.09 mmol), cyclopropylamine (0.48 mmol) and triethylamine (0.066 mL, 0.048 mmol) in NMP (1 mL) was stirred at RT until the reaction had gone to completion. The crude reaction mixture was purified by prep HPLC to give the desired material as a solid (31 mg).

NMR Spectrum: ¹H NMR (400.132 MHz, DMSO-d₆) δ 0.40-0.45 (2H, m), 0.61-0.67 (2H, m), 1.50-1.59 (2H, m), 1.59-1.69 (2H, m), 2.54-2.61 (1H, m), 3.62-3.69 (2H, m), 3.69-3.76 (8H, m), 3.86-3.92 (2H, m), 5.03 (1H, t), 6.44 (1H, s), 6.81 (1H, s), 7.51 (2H, d), 8.21 (2H, d), 8.54 (1H, s)

LCMS Spectrum: m/z (ESI+)(M+H)+=488; HPLC tR=1.61 min.

The following compounds were prepared in an analogous fashion from phenyl N-[4-[4-[1-(2-hydroxyethylsulfonyl)cyclopropyl]-6-morpholin-4-ylpyrimidin-2-yl]phenyl]carbamate and the appropriate amine.

| | | | LCMS | Retention |
|---|---|---|---|---|
| Example | Structure | NAME | MH+ | time (min) |
| 47a | 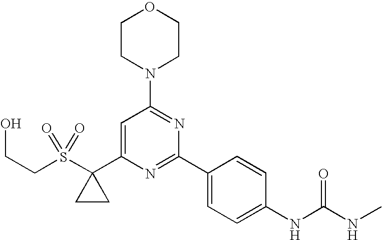 | 1-[4-[4-[1-(2-hydroxyethylsulfonyl)cyclopropyl]-6-morpholin-4-ylpyrimidin-2-yl]phenyl]-3-methylurea | 462 | 1.45 |
| 47b | 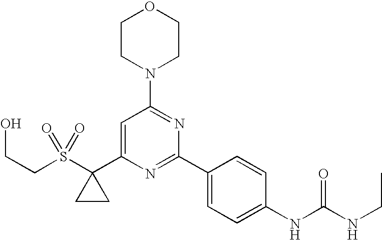 | 3-ethyl-1-[4-[4-[1-(2-hydroxyethylsulfonyl)cyclopropyl]-6-morpholin-4-ylpyrimidin-2-yl]phenyl]urea | 476 | 1.58 |

### EXAMPLE 47a

¹H NMR (400.132 MHz, DMSO-d₆) δ 1.48-1.58 (2H, m), 1.62-1.69 (2H, m), 2.67 (3H, d), 3.61-3.68 (2H, m), 3.69-3.76 (8H, m), 3.85-3.92 (2H, m), 5.03 (1H, s), 6.09 (1H, t), 6.81 (1H, s), 7.50 (2H, d), 8.20 (2H, d), 8.76 (1H, s).

### EXAMPLE 47b

¹H NMR (400.132 MHz, DMSO-d₆) δ 1.07 (3H, t), 1.51-1.57 (2H, m), 1.62-1.68 (2H, m), 3.14 (2H, q), 3.60-3.69 (2H, m), 3.69-3.77 (8H, m), 3.85-3.92 (2H, m), 5.03 (1H, t), 6.16 (1H, t), 6.81 (1H, s), 7.47 (2H, d), 8.20 (2H, d), 8.67 (1H, s).

The preparation of phenyl N-[4-[4-[1-(2-hydroxyethylsulfonyl)cyclopropyl]-6-morpholin-4-ylpyrimidin-2-yl]phenyl]carbamate is described below.

### Phenyl N-[4-[4-[1-(2-hydroxyethylsulfonyl)cyclopropyl]-6-morpholin-4-ylpyrimidin-2-yl]phenyl]carbamate

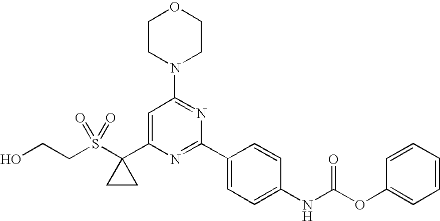

Sodium hydrogen carbonate (65.4 mg, 0.78 mmol) was added to 2-[1-[2-(4-aminophenyl)-6-morpholin-4-ylpyrimidin-4-yl]cyclopropyl]sulfonylethanol (210 mg, 0.52 mmol) in dioxane (8 mL) at 5° C. under a nitrogen atmosphere. Phenyl chloroformate (0.072 mL, 0.57 mmol) was added and the mixture stirred at RT for 18 hours. The reaction mixture was diluted with ethyl acetate (50 mL), the organic layer dried (Na₂SO₄), filtered and evaporated to afford crude product. The crude gum was triturated with a mixture of diethyl ether and isohexane to give the desired material as a beige solid (170 mg).

NMR Spectrum: ¹H NMR (400.132 MHz, DMSO-d₆) δ 1.52-1.60 (2H, m), 1.61-1.70 (2H, m), 3.61-3.79 (1OH, m), 3.82-3.96 (2H, m), 6.85 (1H, s), 7.21-7.34 (3H, m), 7.45 (2H, d), 7.64 (2H, d), 8.31 (2H, d), 10.45 (1H, s)

LCMS Spectrum: m/z (ESI+)(M+H)+=525; HPLC tR=2.40 min.

### 2-[1-[2-(4-Aminophenyl)-6-morpholin-4-ylpyrimidin-4-yl]cyclopropyl]sulfonylethanol

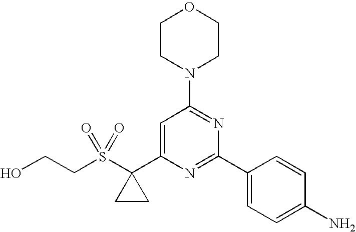

Bis(triphenylphosphine)palladium(II) chloride (48.5 mg, 0.07 mmol) was added to 2-[1-(2-chloro-6-morpholin-4-ylpyrimidin-4-yl)cyclopropyl]sulfonylethoxy-tri(propan-2-yl)silane (520 mg, 1.03 mmol), 4-(4,4,5,5-tetramethyl-1,3,2-dioxaborolan-2-yl)aniline (339 mg, 1.55 mmol) and an aqueous solution of sodium carbonate (1 mL, 2.00 mmol) in a solvent mixture of DMF (5 mL), DME (12 mL), water (1 mL) and ethanol (1 mL) at RT. The atmosphere was replaced with nitrogen and the mixture stirred at 90° C. for 18 hours. The reaction mixture was diluted with ethyl acetate (100 mL), and washed with water (2×100 mL).The organic layer was dried (Na₂SO₄), filtered and evaporated to afford crude product. The crude product was dissolved in DCM then tetrabutylammonium fluoride (5.16 mL, 5.16 mmol) added and the mixture left to stir for 1 hour. A saturated solution of ammonium chloride was added, the layers separated and the organics dried (Na₂SO₄), filtered and evaporated. The crude product was purified by flash silica chromatography, elution gradient 10 to 50% ethyl acetate in isohexane, and the resultant solid further purified by ion exchange chromatography on an SCX column, eluting with 7N ammonia in methanol. The crude solid was triturated with a mixture of diethyl ether and isohexane to give the desired material as a beige solid (210 mg).

NMR Spectrum: ¹H NMR (400.132 MHz, DMSO-d₆) δ 1.47-1.54 (2H, m), 1.59-1.67 (2H, m), 3.59-3.77 (1OH, m), 3.81-3.94 (2H, m), 5.02 (1H, t), 5.57 (2H, s), 6.60 (2H, d), 6.71 (1H, s), 8.04 (2H, d)

LCMS Spectrum: m/z (ESI+)(M+H)+=405; HPLC tR=1.65 min.

### 2-[1-(2-Chloro-6-morpholin-4-ylpyrimidin-4-yl)cyclopropyl]sulfonylethoxy-tri(propan-2-yl)silane

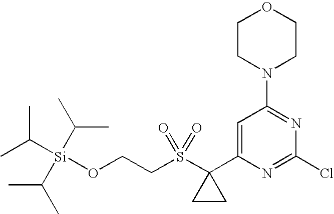

2-[1-(2-Chloro-6-morpholin-4-ylpyrimidin-4-yl)cyclopropyl]sulfonylethanol (550 mg, 1.58 mmol) was added to triisopropylsilyl chloride (0.406 mL, 1.90 mmol) and imidazole (258 mg, 3.80 mmol) in DMF (10 mL) at RT. The resulting solution was stirred under a nitrogen atmosphere overnight. The DMF was removed in vacuo and ethyl acetate added. The solids were removed by filtration and discarded. The filtrate was concentrated in vacuo and purified by flash silica chromatography, elution gradient 0 to 4% methanol in DCM, to give material that was further purified by flash silica chromatography, elution gradient 0-10%ethyl acetate in DCM to give the desired material as a clear gum (700 mg).

NMR Spectrum: ¹H NMR (400.132 MHz, DMSO-d₆) δ 1.02 (18H, d), 1.49-1.53 (2H, m), 1.62-1.66 (2H, m), 2.00 (2H, s), 3.33 (2H, s), 3.62-3.69 (8H, m), 4.03-4.09 (3H, m), 6.95 (1H, s)

LCMS Spectrum: m/z (ESI+)(M+H)+=504; HPLC tR=3.63 min.

### 2-[1-(2-Chloro-6-morpholin-4-ylpyrimidin-4-yl)cyclopropyl]sulfonylethanol

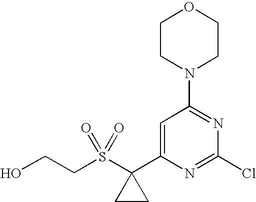

DIPEA (1.052 mL, 6.08 mmol) was added to 2-[1-(2-chloro-6-morpholin-4-ylpyrimidin-4-yl)cyclopropyl]sulfonylacetic acid (1.1 g, 3.04 mmol), in THF (50 mL) at 0° C. under a nitrogen atmosphere. The resulting solution was stirred at 0C for 5 minutes. Ethyl chloroformate (0.349 mL, 3.65 mmol) was added and the reaction stirred at 0° C. for 1 hour. The reaction mixture was filtered and filtrate cooled back down to 0° C. Lithium borohydride (13.68 mL, 27.36 mmol) was added and the mixture warmed to allowed to come to RT. The mixture was cooled back to 0° C. and additional lithium borohydride (13.68 mL, 27.36 mmol) added and the reaction was allowed to come to RT. The mixture was cooled back to 0° C. and additional lithium borohydride (13.68 mL, 27.36 mmol) added and the reaction was allowed to come to RT and stirred for 72 hours. The reaction mixture was adjusted to pH 7 with 2M hydrochloric acid and extracted with ethyl acetate (100 mL). The organic layer was washed with water (100 mL), dried (Na₂SO₄), filtered and evaporated to afford crude product. The crude product was purified by flash silica chromatography, elution gradient 10 to 100% ethyl acetate in isohexane, to give the desired material as a white solid (500 mg).

NMR Spectrum: ¹H NMR (400.132 MHz, DMSO-d₆) δ 1.47-1.52 (2H, m), 1.59-1.67 (2H, m), 3.49-3.57 (2H, m), 3.60-3.70 (8H, m), 3.76-3.86 (2H, m), 4.99 (1H, t), 6.98 (1H, s)

LCMS Spectrum: m/z (ESI+)(M+H)+=348; HPLC tR=1.38 min.

The preparation of 2-[1-(2-chloro-6-morpholin-4-ylpyrimidin-4-yl)cyclopropyl]sulfonylacetic acid was described earlier.

### EXAMPLE 48

### 1-[4-[4-[1-(5-Fluoropyridin-2-yl)sulfonylcyclopropyl]-6-[(3S)-3-methylmorpholin-4-yl]pyrimidin-2-yl]phenyl]-3-methylurea

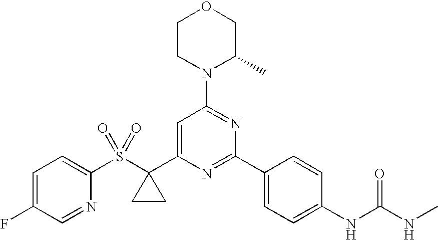

Methylamine (0.509 mL, 1.02 mmol) was added to phenyl N-[4-[4-[1-(5-fluoropyridin-2-yl)sulfonylcyclopropyl]-6-[(3S)-3-methylmorpholin-4-yl]pyrimidin-2-yl]phenyl]carbamate (200 mg, 0.34 mmol) and triethylamine (0.141 mL, 1.02 mmol) in DMF (1.7 mL) and the reaction stirred at 50° C. for 2 hours. The crude product was purified by prep HPLC to give the desired material as a white solid (70.0 mg).

NMR Spectrum: ¹H NMR (400.132 MHz, DMSO-d₆) δ 1.18 (3H, d), 1.69-1.72 (2H, m), 1.96-1.99 (2H, m), 2.66 (3H, d), 3.12-3.18 (1H, m), 3.43-3.48 (1H, m), 3.60 (1H, dd), 3.74 (1H, d), 3.95 (1H, d), 4.15 (1H, d), 4.45 (1H, s), 6.02-6.04 (1H, m), 6.68 (1H, s), 7.37 (2H, d), 7.68 (2H, d), 7.95-8.00 (1H, m), 8.03-8.06 (1H, m), 8.72 (1H, s), 8.87 (1H, d).

LCMS Spectrum: m/z (ESI+)(M+H)+=527; HPLC tR=1.61 min.

mTOR Kinase Assay (Echo): 0.00252 μM

The following compound was prepared in an analogous fashion from phenyl N-[4-[4-[1-(5-fluoropyridin-2-yl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 48a | 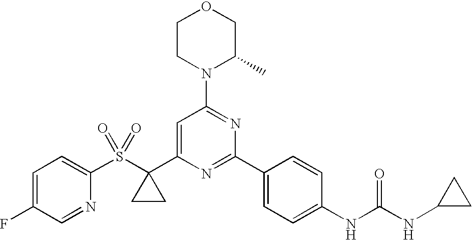 | 3-cyclopropyl-1-[4-[4-[1-(5-fluoropyridin-2-yl)sulfonylcyclopropyl]-6-[(3S)-3-methylmorpholin-4-yl]pyrimidin-2-yl]phenyl]urea | 553 | 2.43 |

### EXAMPLE 48a

¹H NMR (400.132 MHz, DMSO-d₆) δ 1.66-1.70 (2H, m), 1.96-1.99 (2H, m), 3.69 (8H, s), 3.79 (3H, s), 6.74 (1H, s), 7.40 (3H, d), 7.66 (2H, d), 7.77-7.78 (3H, m), 8.35 (1H, s), 8.80 (1H, s), 8.87 (2H, d).

mTOR Kinase Assay (Echo): 0.00103 μM

The preparation of phenyl N-[4-[4-[1-(5-fluoropyridin-2-yl)sulfonylcyclopropyl]-6-[(3S)-3-methylmorpholin-4-yl]pyrimidin-2-yl]phenyl]carbamate is described below:

### Phenyl N-[4-[4-[1-(5-fluoropyridin-2-yl)sulfonylcyclopropyl]-6-[(3S)-3-methylmorpholin-4-yl]pyrimidin-2-yl]phenyl]carbamate

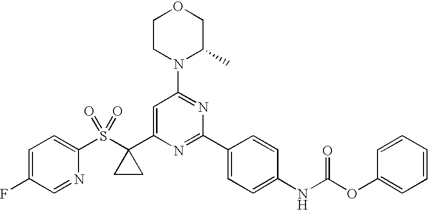

Sodium bicarbonate (0.403 g, 4.79 mmol) was added to 4-[4-[1-(5-fluoropyridin-2-yl)sulfonylcyclopropyl]-6-[(3S)-3-methylmorpholin-4-yl]pyrimidin-2-yl]aniline (1.5 g, 3.19 mmol), in 1,4-dioxane (15.97 mL), followed by the dropwise addition of phenyl chloroformate (0.402 mL, 3.19 mmol) over 2 minutes and the reaction stirred at RT for 2 hours. The reaction mixture was evaporated to dryness, redissolved in DCM (20 mL), the organics washed with water (20 mL), dried (MgSO₄), filtered and evaporated to give the desired material (2.0 g) which was used without further purification.

NMR Spectrum: ¹H NMR (400.132 MHz, DMSO-d₆) δ 1.19 (3H, d), 1.71-1.76 (2H, m), 1.96-1.99 (2H, m), 3.14-3.22 (1H, m), 3.42-3.49 (1H, m), 3.58-3.62 (1H, m), 3.75 (1H, d), 3.94-3.98 (1H, m), 4.19 (1H, s), 4.48 (1H, s), 6.74 (1H, s), 7.24-7.26 (2H, m), 7.45 (2H, t), 7.54 (2H, d), 7.80 (2H, d), 7.99 (1H, dt), 8.07 (1H, dd), 8.89 (1H, d), 10.48 (1H, s)

LCMS Spectrum: m/z (ESI+) (M+H)+=590; HPLC tR=2.95 min.

### 4-[4-[1-(5-Fluoropyridin-2-yl)sulfonylcyclopropyl]-6-[(3S)-3-methylmorpholin-4-yl]pyrimidin-2-yl]aniline

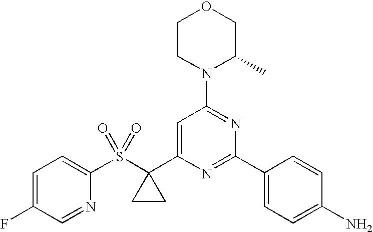

Bis(triphenylphosphine)palladium(II) chloride (0.136 g, 0.19 mmol) was added to 4-(4,4,5,5-tetramethyl-1,3,2-dioxaborolan-2-yl)aniline (1.104 g, 5.04 mmol) and 2-chloro-4-[1-(5-fluoropyridin-2-yl)sulfonylcyclopropyl]-6-[(3S)-3-methylmorpholin-4-yl]pyrimidine (1.6 g, 3.88 mmol) and an aqueous solution of sodium carbonate (5.81 mL, 11.63 mmol) in a solvent mixture of DMF (0.564 mL), ethanol (4.70 mL) and water (4.70 mL) at RT under an atmosphere of nitrogen. The resulting mixture was stirred at 85° C. for 4 hours. The reaction mixture was diluted with ethyl acetate (20 mL), and the organics washed with water (2×20 mL), dried (MgSO₄), filtered and evaporated to afford crude product. The crude product was purified by flash silica chromatography, elution gradient 0 to 10% 3.5N methanolic ammonia in DCM, to give the desired material as a cream solid (1.5 g).

NMR Spectrum: ¹H NMR (400.132 MHz, DMSO-d₆) δ 1.16 (3H, d), 1.65-1.70 (2H, m), 1.94-1.97 (2H, m), 3.12 (1H, dt), 3.44 (1H, dt), 3.59 (1H, dd), 3.73 (1H, d), 3.93 (1H, dd), 4.11-4.13 (1H, m), 4.41 (1H, s), 6.46 (2H, d), 6.58 (1H, s), 7.49 (2H, d), 7.98 (2H, dt), 8.04 (2H, dd), 8.89 (1H, d)

LCMS Spectrum: m/z (ESI+) (M+H)+=470; HPLC tR=2.30 min.

### 2-Chloro-4-[1-(5-fluoropyridin-2-yl)sulfonylcyclopropyl]-6-[(3S)-3-methylmorpholin-4-yl]pyrimidine

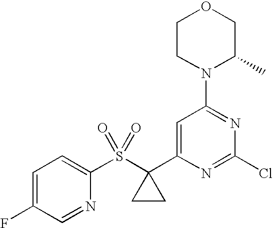

1,2-Dibromoethane (1.025 mL, 11.89 mmol) was added to 2-chloro-4-[(5-fluoropyridin-2-yl)sulfonylmethyl]-6-[(3S)-3-methylmorpholin-4-yl]pyrimidine (2.3 g, 5.95 mmol), an aqueous solution of sodium hydroxide (2.97 mL, 29.73 mmol) and tetrabutylammonium bromide (0.383 g, 1.19 mmol) in toluene (29.7 mL) and the resulting solution stirred at 60° C. for 3 hours. The reaction mixture was evaporated to dryness, redissolved in ethyl acetate (50 mL), and the organics washed sequentially with water (50 mL) and saturated brine (50 mL). The organic layer was dried (MgSO₄), filtered and evaporated to afford crude product. The crude product was purified by flash silica chromatography, elution gradient 5 to 50% ethyl acetate in DCM, to give the desired material as a white solid (1.8 g).

NMR Spectrum: ¹H NMR (400.132 MHz, DMSO-d₆) δ 1.14 (3H, d), 1.63-1.66 (2H, m), 1.88-1.93 (2H, m), 3.11-3.17 (1H, m), 3.39 (1H, dt), 3.54 (1H, dd), 3.69 (1H, d), 3.91 (2H, dd), 4.27 (1H, s), 6.81 (1H, s), 8.02-8.11 (2H, m), 8.83 (1H, d)

LCMS Spectrum: m/z (ESI+)(M+H)+ 413, HPLC tR=2.10 min

### 2-Chloro-4-[(5-fluoropyridin-2-yl)sulfonylmethyl]-6-[(3S)-3-methylmorpholin-4-yl]pyrimidine

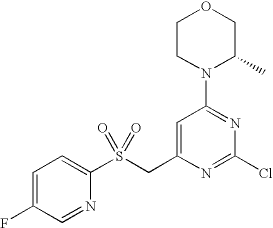

3-Chloroperoxybenzoic acid (5.79 g, 25.15 mmol) was added portionwise to 2-chloro-4-[(5-fluoropyridin-2-yl)sulfanylmethyl]-6-[(3S)-3-methylmorpholin-4-yl]pyrimidine (3.57 g, 10.06 mmol), in DCM (50.3 mL) and the reaction stirred at RT for 2 hours. The reaction mixture was washed with a saturated solution of sodium hydrogen carbonate (50 mL), the organic layer separated, dried (MgSO₄), filtered and evaporated to afford crude product. The crude product was purified by flash silica chromatography, elution gradient 0 to 60% ethyl acetate in DCM, to give the desired material as a white solid (2.3 g).

NMR Spectrum: ¹H NMR (400.132 MHz, DMSO-d₆) δ 1.17 (3H, d), 3.17 (1H, t), 3.42 (1H, dt), 3.57 (1H, dd), 3.71 (1H, d), 3.92 (2H, dd), 4.17 (1H, s), 4.74 (2H, d), 6.82 (1H, s), 8.01-8.09 (2H, m), 8.89 (1H, d)

LCMS Spectrum: m/z (ESI+)(M+H)+ 387, HPLC tR=1.88 min

### 2-Chloro-4-[(5-fluoropyridin-2-yl)sulfanylmethyl]-6-[(3S)-3-methylmorpholin-4-yl]pyrimidine

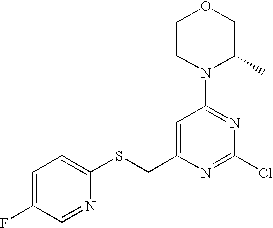

Potassium hydroxide (3.22 g, 57.33 mmol) was added to (5-fluoropyridin-2-yl)dimethylaminomethanedithioate (3.1 g, 14.33 mmol) in ethanol (71.7 mL) and the resulting solution heated at 65° C. for 4 hours. The reaction was cooled, 2-chloro-4-(iodomethyl)-6-[(3S)-3-methylmorpholin-4-yl]pyrimidine (7.09 g, 20.06 mmol) added and the reaction stirred at RT for 4 hours. Water (50 mL) was added and the reaction extracted with DCM (2×100 mL). The organics were dried (MgSO₄), filtered and concentrated to give crude product which was purified by flash silica chromatography, elution gradient 0 to 50% ethyl acetate in DCM, to give the desired material as a beige gum (3.57 g).

NMR Spectrum: ¹H NMR (400.132 MHz, DMSO-d₆) δ 1.13 (3H, d), 3.11-3.17 (1H, m), 3.41 (1H, dt), 3.54-3.57 (1H, m), 3.69 (1H, d), 3.90 (2H, dd), 4.24-4.26 (1H, m), 4.29 (2H, d), 6.84 (1H, s), 7.45 (1H, dd), 7.68 (1H, dt), 8.50 (1H, d)

LCMS Spectrum: m/z (ESI+)(M+H)+ 355, HPLC tR=2.38 min

### (5-Fluoropyridin-2-yl)dimethylaminomethanedithioate

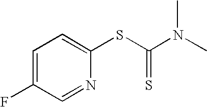

2-Bromo-5-fluoropyridine (4 g, 22.73 mmol) was added portionwise to isopropylmagnesium chloride-lithium chloride complex (14% in THF, 23 mL, 22.73 mmol), at 0° C., over a period of 2 minutes under a nitrogen atmosphere. The resulting solution was warmed to RT over a period of 2 hours then cooled back to 0° C. and tetramethylthiuram disulfide (5.46 g, 22.73 mmol) in DCM (22.73 mL) added. The reaction was warmed to RT and stirred for 6 hours. The reaction was poured into a saturated aqueous solution of ammonium chloride (100 mL) and the aqueous layer extracted with DCM (2×100 mL). The organics were dried (MgSO₄), concentrated in vacuo and the crude product was purified by flash silica chromatography, elution gradient 0 to 60% ethyl acetate in DCM, to give the desired material as a brown oil which solidified on standing (3.10 g).

NMR Spectrum: ¹H NMR (400.132 MHz, DMSO-d₆) δ 3.45 (3H, s), 3.46 (3H, s), 7.70 (1H, dd), 7.86 (1H, dt), 8.65 (1H, d)

LCMS Spectrum: m/z (ESI+)(M+H)+ 217, HPLC tR=1.70 min

The preparation of 2-chloro-4-(iodomethyl)-6-[(3S)-3-methylmorpholin-4-yl]pyrimidine was described earlier.

### EXAMPLE 49

### N,N-Dimethyl-6-[1-[2-[4-(methylcarbamoylamino)phenyl]-6-[(3S)-3-methylmorpholin-4-yl]pyrimidin-4-yl]cyclopropyl]sulfonylpyridine-3-carboxamide

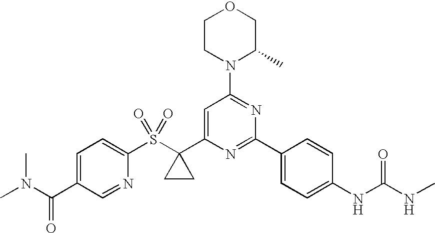

Methylamine (0.250 mL, 0.50 mmol) was added to phenyl N-[4-[4-[1-[5-(dimethylcarbamoyl)pyridin-2-yl]sulfonylcyclopropyl]-6-[(3S)-3-methylmorpholin-4-yl]pyrimidin-2-yl]phenyl]carbamate (0.064 g, 0.10 mmol) and triethylamine (0.042 mL, 0.30 mmol) in DMF (2 mL) and the resulting solution stirred at 50° C. for 18 hours. The reaction was cooled and the mixture purified by preparative HPLC to give the desired material as a white solid (0.03 g).

NMR Spectrum: ¹H NMR (400.132 MHz, DMSO-d₆) δ δ 1.16-1.18 (3H, d), 1.73-1.76 (2H, m), 1.99-2.02 (2H, m), 2.65-2.66 (3H, d), 2.88 (3H, s), 3.05 (3H, s), 3.05-3.17 (1H, td), 3.41-3.48 (1H, td), 3.58-3.62 (1H, dd), 3.73-3.76 (1H, d), 3.93-3.97 (1H, dd), 4.15 (1H, bs), 4.44 (1H, bs), 6.07-6.10 (1H, q), 6.65 (1H, s), 7.37-7.39 (2H, d), 7.67-7.69 (2H, d), 8.00-8.01 (1H, d), 8.11-8.13 (1H, dd), 8.72 (1H, s), 8.87-8.88 (1H, dd).

LCMS Spectrum: m/z (ES+) (M+H)+=580; HPLC tR=1.88 min.

mTOR Kinase Assay (Echo): 0.00187 μM

The compound shown in table below was prepared in an analogous manner from phenyl N-[4-[4-[1-[5-(dimethylcarbamoyl)pyridin-2-yl]sulfonylcyclopropyl]-6-[(3S)-3-methylmorpholin-4-yl]pyrimidin-2-yl]phenyl]carbamate using the appropriate amine.

| Ex- | | | LCMS | Retention |
|---|---|---|---|---|
| ample | Structure | NAME | MH+ | time (min) |
| 49a | 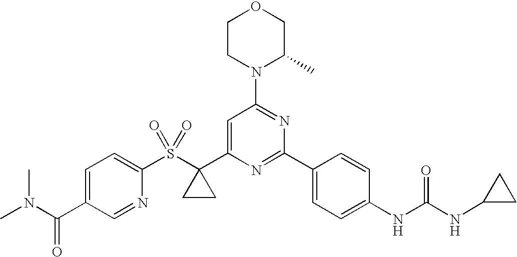 | 6-[1-[2-[4-(cyclopropyl-carbamoylamino)phenyl]-6-[(3S)-3-methyl-morpholin-4-yl]pyrimidin-4-yl]cyclopropyl]sulfonyl-N,N-dimethylpyridine-3-carboxamide | 606 | 2.00 |

### EXAMPLE 49a

¹H NMR (400.132 MHz, DMSO-d₆) δ 0.39-0.43 (2H, m), 0.62-0.67 (2H, m) 1.16-1.18 (3H, d), 1.73-1.76 (2H, m), 2.00-2.02 (2H, m), 2.88 (3H, s), 3.05 (3H, s) 3.11-3.18 (1H, td), 3.42-3.48 (1H, td), 3.58-3.62 (1H, dd), 3.73-3.76 (1H, d), 3.93-3.97 (1H, dd), 4.13 (1H, bs), 4.44 (1H, bs), 6.44-6.45 (1H, d), 6.66 (1H, s), 7.37-7.39 (2H, d), 7.67-7.69 (2H, d), 8.00-8.03 (1H, d), 8.11-8.13 (1H, dd), 8.51 (1H, s), 8.87-8.88 (1H, dd).

mTOR Kinase Assay (Echo): 0.00267 μM

The preparation of phenyl N-[4-[4-[1-[5-(dimethylcarbamoyl)pyridin-2-yl]sulfonylcyclopropyl]-6-[(3S)-3-methylmorpholin-4-yl]pyrimidin-2-yl]phenyl]carbamate is described below.

### Phenyl N-[4-[4-[1-[5-(dimethylcarbamoyl)pyridin-2-yl]sulfonylcyclopropyl]-6-[(3S)-3-methylmorpholin-4-yl]pyrimidin-2-yl]phenyl]carbamate

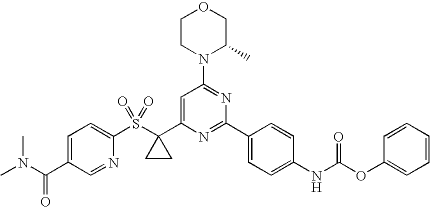

Phenyl chloroformate (0.015 mL, 0.12 mmol) was added dropwise to 6-[1-[2-(4-aminophenyl)-6-[(3S)-3-methylmorpholin-4-yl]pyrimidin-4-yl]cyclopropyl]sulfonyl-N,N-dimethylpyridine-3-carboxamide (0.064 g, 0.12 mmol) and sodium hydrogen carbonate (0.015 g, 0.18 mmol) in dioxane (7.5 mL) and the resulting solution stirred at RT for 1 hour. The material was purified by flash silica chromatography, elution gradient 0 to 40% ethyl acetate in DCM, to give the desired material.

NMR Spectrum: ¹H NMR (400.132 MHz, CDCl₃) δ 1.29-1.31 (3H, d), 1.66-1.74 (2H, m), 2.17-2.19 (2H, m), 2.88 (3H, s), 3.12 (3H, s), 3.24-3.31 (1H, td), 3.54-3.61 (1H, td), 3.70-3.74 (1H, dd), 3.80-3.83 (1H, d), 4.01-4.05 (1H, dd), 4.16-4.17 (1H, bs), 4.42 (1H, bs), 6.79 (1H, s), 16-7.26 (2H, d), 7.22-7.26 (1H, t), 7.37-7.41 (2H, t), 7.47-7.49 (2H, d), 7.64-7.69 (1H, m), 7.84-7.87 (1H, dd), 7.95-7.97 (3H, m), 8.75-8.76 (1H, d).

LCMS Spectrum: m/z (ES+) (M+H)+=643; HPLC tR=2.62 min.

### 6-[1-[2-(4-Aminophenyl)-6-[(3S)-3-methylmorpholin-4-yl]pyrimidin-4-yl]cyclopropyl]sulfonyl-N,N-dimethylpyridine-3-carboxamide

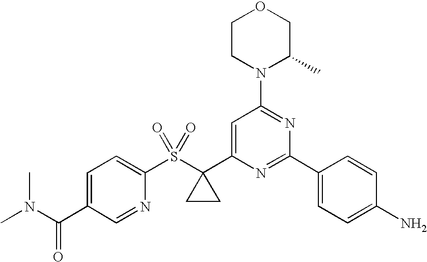

Bis(triphenylphosphine)palladium (II) chloride (0.015 g, 0.02 mmol) was added to 6-[1-[2-Chloro-6-[(3S)-3-methylmorpholin-4-yl]pyrimidin-4-yl]cyclopropyl]sulfonyl-N,N-dimethylpyridine-3-carboxamide (0.203 g, 0.44 mmol), 4-(4,4,5,5-tetramethyl-1,3,2-dioxaborolan-2-yl)aniline (0.096 g, 0.44 mmol) and an aqueous solution of sodium carbonate (1.09 mL, 2.18 mmol) in a solvent mixture (2 mL) (the solvent mixture comprised 18% DMF, 82% of a 7:3:2 mixture of DME:water:Ethanol) at RT under a nitrogen atmosphere. The resulting solution was stirred at 80° C. for 3 hours. The reaction was cooled, water added and the solids removed by filtration. The filtrate was extracted twice with ethyl acetate and the combined organics dried (MgSO₄), filtered and evaporated. The solids from the filtration were combined with those from the extraction to give the desired material which was used without further purification.

LCMS Spectrum: m/z (ES+) (M+H)+=523; HPLC tR=1.98 min.

### 6-[1-[2-Chloro-6-[(3S)-3-methylmorpholin-4-yl]pyrimidin-4-yl]cyclopropyl]sulfonyl-N,N-dimethylpyridine-3-carboxamide

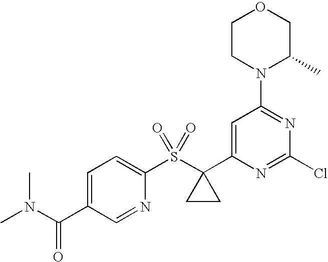

Sodium hydroxide (3.36 g, 83.96 mmol) in water (3.6 mL) was added to 6-[[2-chloro-6-[(3S)-3-methylmorpholin-4-yl]pyrimidin-4-yl]methylsulfonyl]-N,N-dimethylpyridine-3-carboxamide (0.666 g, 1.51 mmol), 1,2-dibromoethane (0.652 mL, 7.56 mmol) and tetrabutylammonium bromide (0.049 g, 0.15 mmol) in DCM (20 mL) and the resulting solution stirred at RT for 18 hours. The reaction mixture was diluted with water and extracted with DCM. The organics were washed with saturated brine, dried (MgSO₄), filtered and evaporated to afford crude product. The crude product was purified by flash silica chromatography, elution gradient 0 to 7% methanol (contaning 0.1% ammonia) in DCM, to give the desired material as a brown gum (0.406 g).

NMR Spectrum: ¹H NMR (400.132 MHz, CDCl₃) δ 1.27-1.29 (3H, d), 1.61-1.68 (2H, m), 2.07-2.10 (2H, m), 3.00 (3H, s), 3.14 (3H, s), 3.20-3.28 (1H, td), 3.48-3.54 (1H, td), 3.64-3.67 (1H, dd), 3.75-3.78 (1H, d), 3.96-4.02 (2H, m), 4.26 (1H, bs), 6.95 (1H, s), 7.93-7.99 (2H, m), 8.71-8.72 (1H, d).

LCMS Spectrum: m/z (ES+) (M+H)+=466; HPLC tR=1.71 min.

### 6-[[2-Chloro-6-[(3S)-3-methylmorpholin-4-yl]pyrimidin-4-yl]methylsulfonyl]-N,N-dimethylpyridine-3-carboxamide

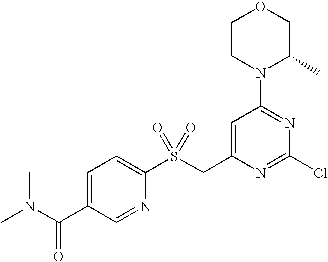

6-[[2-Chloro-6-[(3S)-3-methylmorpholin-4-yl]pyrimidin-4-yl]methylsulfanyl]-N,N-dimethylpyridine-3-carboxamide (1.47 g, 3.60 mmol) was dissolved in dioxane (45 mL) and 2N sulfuric acid (0.11 mL) added. The solution was heated to 55° C., a solution of sodium tungstate dihydrate (0.024 g, 0.07 mmol) in water (1.08 mL) added and the solution allowed to stir for 10 minutes. Hydrogen peroxide (2.229 mL, 72.07 mmol) was then added dropwise over several minutes and the solution heated at 55° C. for 3 hours. Water was added and the reaction was allowed to cool. The aqueous solution was extracted with DCM, the organics dried (MgSO₄), filtered and evaporated. The crude product was purified by flash silica chromatography, elution gradient 0 to 40% ethyl acetate in DCM, to give the desired material as a cream solid (1.45 g).

NMR Spectrum: ¹H NMR (400.132 MHz, CDCl₃) δ 1.31-1.33 (3H, d), 3.04 (3H, s), 3.17 (3H, s), 3.24-3.32 (1H, td), 3.50-3.57 (1H, td), 3.66-3.70 (1H, dd), 3.78-3.80 (1H, d), 3.99-4.03 (2H, m), 4.26 (1H, bs), 4.58 (2H, s), 6.52 (1H, s), 7.97-8.03 (2H, m), 8.82-8.83 (1H, m).

LCMS Spectrum: m/z (ES+) (M+H)+=440; HPLC tR=1.59 min.

### 6-[[2-Chloro-6-[(3S)-3-methylmorpholin-4-yl]pyrimidin-4-yl]methylsulfanyl]-N,N-dimethylpyridine-3-carboxamide

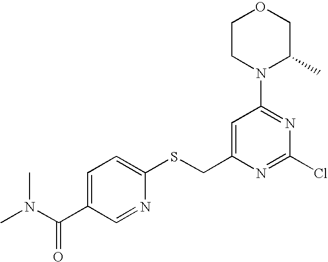

DIPEA (1.043 mL, 5.99 mmol) was added to 6-[[2-chloro-6-[(3S)-3-methylmorpholin-4-yl]pyrimidin-4-yl]methylsulfanyl]pyridine-3-carboxylic acid (1.369 g, 3.59 mmol) and HATU (1.366 g, 3.59 mmol) in DMA (10 mL) and the resulting solution stirred at RT for 15 minutes. Dimethylamine (1.497 mL, 2.99 mmol) was added and the reaction allowed to stir for 2.5 hours. Water was added to the solution and the solution extracted with ethyl acetate. The ethyl acetate was washed with a saturated aqueous solution of sodium bicarbonate, dried (MgSO₄), filtered and evaporated to give the desired material which was used without further purification.

LCMS Spectrum: m/z (ES+) (M+H)+=408; HPLC tR=1.83 min.

### 6-[[2-Chloro-6-[(3S)-3-methylmorpholin-4-yl]pyrimidin-4-yl]methylsulfanyl]pyridine-3-carboxylic acid

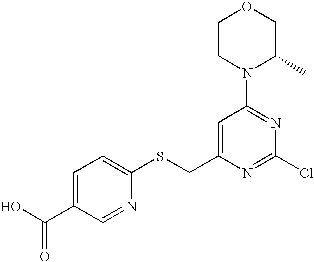

2-Chloro-4-(iodomethyl)-6-[(3S)-3-methylmorpholin-4-yl]pyrimidine (2.0 g, 5.66 mmol) was added to 6-mercaptonicotinic acid (1.317 g, 8.48 mmol) and DIPEA (2.463 mL, 14.14 mmol) in acetonitrile (100 mL) and the resulting solution stirred at RT for 2 hours. The solvent was removed under vacuum and the residue dissolved in DCM. The organics were washed sequentially with water and saturated brine, dried (MgSO₄), filtered and evaporated to afford crude product. The crude product was purified by flash silica chromatography, elution gradient 0 to 10% methanol (containing 0.1% ammonia) in DCM, to give the desired material as a brown solid (1.79 g).

NMR Spectrum: ¹H NMR (400.132 MHz, CDCl₃) δ 1.19-1.20 (3H, d), 3.16-3.21 (1H, t), 3.40-3.47 (1H, m), 3.58-3.61 (2H, dd), 3.91-3.94 (2H, d), 4.25 (3H, bs), 6.08 (1H, bs), 6.52 (1H, s), 7.01 (1H, bs), 7.89 (1H, bs), 8.89 (1H, bs).

LCMS Spectrum: m/z (ES+) (M+H)+=381; HPLC tR=0.83 min.

The preparation of 2-chloro-4-(iodomethyl)-6-[(3S)-3-methylmorpholin-4-yl]pyrimidine was described earlier.

### EXAMPLE 50

### N,N-Dimethyl-3-[1-[2-[4-(methylcarbamoylamino)phenyl]-6-[(3S)-3-methylmorpholin-4-yl]pyrimidin-4-yl]cyclopropyl]sulfonylpyridine-2-carboxamide

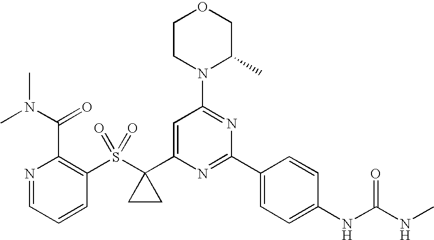

Methylamine (0.441 mL, 0.88 mmol) was added to phenyl N-[4-[4-[1-[2-(dimethylcarbamoyl)pyridin-3-yl]sulfonylcyclopropyl]-6-[(3S)-3-methylmorpholin-4-yl]pyrimidin-2-yl]phenyl]carbamate (0.113 g, 0.18 mmol) and triethylamine (0.074 mL, 0.53 mmol) in DMF (2 mL) and the resulting solution stirred at 50° C. for 2 hours. The reaction was cooled and purified by preparative HPLC, to give the desired material as a colourless gum (11 mg).

NMR Spectrum: ¹H NMR (400.132 MHz, DMSO-d₆) δ 1.16-1.18 (3H, d), 1.62 (2H, bs), 1.98 (2H, bs), 2.55 (3H, s), 2.66 (3H, s), 2.96 (3H, s), 3.13-3.19 (1H, td), 3.41-3.47 (1H, td), 3.58-3.61 (1H, dd), 3.73-3.76 (1H, d), 3.94-3.98 (1H, dd), 4.16 (1H, bs), 4.47 (1H, bs), 6.12 (1H, bs), 6.73 (1H, s), 7.43-7.46 (2H, d), 7.59-7.62 (1H, q), 7.85-7.87 (2H, d), 8.16-8.18 (1H, d), 8.82 (1H, bs), 8.85-8.86 (1H, dd).

LCMS Spectrum: m/z (ES+) (M+H)+=580; HPLC tR=1.76 min.

mTOR Kinase Assay (Echo): 0.0104 μM

The following compound was prepared in an analogous fashion from phenyl N-[4-[4-[1-[2-(dimethylcarbamoyl)pyridin-3-yl]sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 50a | 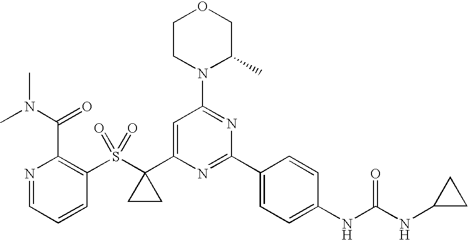 | 3-[1-[2-[4-(cyclopropylcarbamoylamino)phe-nyl]-6-[(3S)-3-methylmorpholin-4-yl]pyrimidin-4-yl]cyclopropyl]sulfonyl-N,N-dimethylpyridine-2-carboxamide | 606 | 1.92 |

### EXAMPLE 50a

¹H NMR (400.132 MHz, DMSO-d₆) δ 0.40-0.44 (2H, m), 0.63-0.67 (2H, m) 1.16-1.17 (3H, d), 1.60 (2H, bs), 1.96 (2H, bs), 2.55 (3H, s), 2.97 (3H, s), 3.10-3.17 (1H, td), 3.42-3.48 (1H, td), 3.58-3.62 (1H, dd), 3.73-3.76 (1H, d), 3.94-3.97 (1H, dd), 4.15 (1H, bs), 4.45 (1H, bs), 6.45-6.46 (1H, d), 6.72 (1H, s), 7.41-7.43 (2H, d), 7.57-7.60 (1H, q), 7.84-7.86 (2H, d), 8.11-8.14 (1H, dd), 8.55 (1H, s), 8.84-8.85 (1H, dd).

mTOR Kinase Assay (Echo): 0.00794 μM

The preparation of phenyl N-[4-[4-[1-[2-(dimethylcarbamoyl)pyridin-3-yl]sulfonylcyclopropyl]-6-[(3S)-3-methylmorpholin-4-yl]pyrimidin-2-yl]phenyl]carbamate is described below.

### Phenyl N-[4-[4-[1-[2-(dimethylcarbamoyl)pyridin-3-yl]sulfonylcyclopropyl]-6-[(3S)-3-methylmorpholin-4-yl]pyrimidin-2-yl]phenyl]carbamate

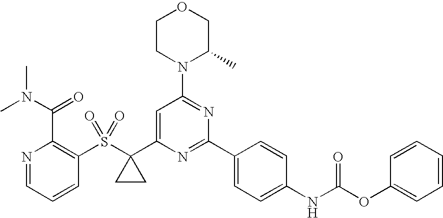

Phenyl chloroformate (0.071 mL, 0.57 mmol) was added dropwise to 3-[1-[2-(4-aminophenyl)-6-[(3S)-3-methylmorpholin-4-yl]pyrimidin-4-yl]cyclopropyl]sulfonyl-N,N-dimethylpyridine-2-carboxamide (0.296 g, 0.57 mmol) and sodium hydrogen carbonate (0.071 g, 0.85 mmol) in dioxane (18 mL) and the resulting solution stirred at RT for 3 hours. The solids were removed by filtration and the filtrate purified by flash silica chromatography, elution gradient 0 to 100% ethyl acetate in DCM, to give additional desired material as a yellow gum (0.227 g).

NMR Spectrum: ¹H NMR (400.132 MHz, CDCl₃) δ 1.22-1.24 (3H, d), 1.53-1.60 (2H, m), 2.13-2.17 (2H, m), 2.77 (3H, s), 3.14 (3H, s), 3.18-3.25 (1H, td), 3.50-3.57 (1H, td) 3.66-3.70 (1H, dd), 3.77-3.80 (1H, d), 3.98-3.99 (1H, dd), 4.08 (1H, bs), 4.38 (1H, bs), 6.69 (1H, s), 7.16-7.18 (2H, d), 7.21-7.25 (2H, m), 7.36-7.40 (2H, t), 7.48-7.50 (2H, d), 7.67 (1H, bs), 7.94-7.96 (1H, dd), 8.15-8.17 (2H, d), 8.70-8.72 (1H, dd).

LCMS Spectrum: m/z (ES+) (M+H)+=643; HPLC tR=3.09 min.

### 3-[1-[2-(4-Aminophenyl)-6-[(3S)-3-methylmorpholin-4-yl]pyrimidin-4-yl]cyclopropyl]sulfonyl-N,N-dimethylpyridine-2-carboxamide

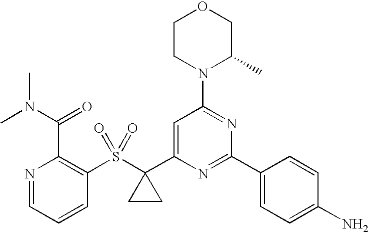

Bis(triphenylphosphine)palladium (II) chloride (0.038 g, 0.05 mmol) was added to 3-[1-[2-chloro-6-[(3S)-3-methylmorpholin-4-yl]pyrimidin-4-yl]cyclopropyl]sulfonyl-N,N-dimethylpyridine-2-carboxamide (0.503 g, 1.08 mmol), 4-(4,4,5,5-tetramethyl-1,3,2-dioxaborolan-2-yl)aniline (0.237 g, 1.08 mmol) and an aqueous solution of sodium carbonate (2.70 mL, 5.40 mmol) in a solvent mixture (2 mL) (the solvent mixture comprised 18% DMF, 82% of a 7:3:2 mixture of DME:Water:Ethanol) and the resulting solution stirred at 80° C. for 15 hours. The reaction was cooled to RT and water was added. The solids were filtered to give the desired material. The filtrate was extracted with ethyl acetate and the organics dried (MgSO₄), filtered and evaporated to dryness to yield an additional sample of the desired material. Both crops of the desired material were combined and used without further purification.

LCMS Spectrum: m/z (ES+) (M+H)+=523; HPLC tR=1.85 min.

### 3-[1-[2-Chloro-6-[(3S)-3-methylmorpholin-4-yl]pyrimidin-4-yl]cyclopropyl]sulfonyl-N,N-dimethylpyridine-2-carboxamide

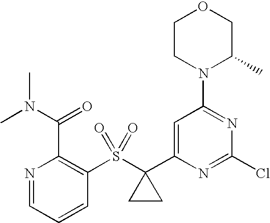

Sodium hydroxide (2.373 g, 59.32 mmol) in water (2.373 mL) was added to 3-[[2-chloro-6-[(3S)-3-methylmorpholin-4-yl]pyrimidin-4-yl]methylsulfonyl]-N,N-dimethylpyridine-2-carboxamide (0.475 g, 1.08 mmol), 1,2-dibromoethane (0.465 mL, 5.39 mmol) and tetrabutylammonium bromide (0.035 g, 0.11 mmol) in DCM and the resulting solution stirred at RT for 18 hours. Water was added and the solution was extracted with DCM. The organic layer was dried (MgSO₄) and filtered to give the desired material (0.539 g).

LCMS Spectrum: m/z (ES+) (M+H)+=466; HPLC tR=1.64 min.

### 3-[[2-chloro-6-[(3S)-3-methylmorpholin-4-yl]pyrimidin-4-yl]methylsulfonyl]-N,N-dimethylpyridine-2-carboxamide

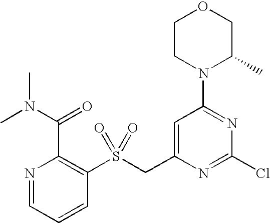

3-[[2-Chloro-6-[(3S)-3-methylmorpholin-4-yl]pyrimidin-4-yl]methylsulfanyl]-N,N-dimethylpyridine-2-carboxamide (0.533 g, 1.31 mmol) was dissolved in dioxane (15 mL) and 2N sulfuric acid (0.041 mL) was added. The solution was heated to 55° C. and a solution of sodium tungstate dihydrate (8.62 mg, 0.03 mmol) in water (0.4 mL) added and the solution allowed to stir for 10 minutes. Hydrogen peroxide (0.808 mL, 26.13 mmol) was then added dropwise over several minutes. The solution was heated at 55° C. for 5.5 hours. The heat was removed and the reaction was allowed to stir at RT overnight. Water was added and the reaction was allowed to cool. The aqueous solution was extracted with DCM. The organic layer was dried (MgSO₄), filtered and evaporated. The crude product was purified by flash silica chromatography, elution gradient 0 to 7% methanil (containing 0.1% ammonia) in DCM, to give the desired material as a yellow gum (0.475 g).

LCMS Spectrum: m/z (ES+) (M+H)+=440; HPLC tR=1.62 min.

### 3-[[2-Chloro-6-[(3S)-3-methylmorpholin-4-yl]pyrimidin-4-yl]methylsulfanyl]-N,N-dimethylpyridine-2-carboxamide

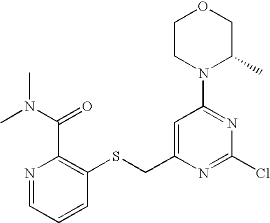

2-Chloro-4-(iodomethyl)-6-[(3S)-3-methylmorpholin-4-yl]pyrimidine (1.009 g, 2.85 mmol) was added to N,N-dimethyl-3-sulfanylpyridine-2-carboxamide (0.520 g, 2.85 mmol) and DIPEA (0.746 mL, 4.28 mmol) in acetonitrile (20 mL) and the resulting solution stirred at RT for 2 hours. Further DIPEA (0.746 mL, 4.28 mmol) was added and the reaction was allowed to stir for several over a weekend. The reaction was heated at 40° C. for 7 days. The solvent was removed and the residue dissolved in DCM and washed with water. The organic layer was dried (MgSO₄), filtered and evaporated to afford crude product. The crude product was purified by ion exchange chromatography using an SCX column, eluting with 7M ammonia in methanol, to give material which was further purified by flash silica chromatography, elution gradient 0 to 7% methanol in DCM, to give the desired material as a brown gum (0.533 g).

LCMS Spectrum: m/z (ES+) (M+H)+=408; HPLC tR=1.57 min.

### N,N-Dimethyl-3-sulfanylpyridine-2-carboxamide

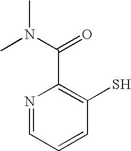

DIPEA (2.164 mL, 12.42 mmol) was added to 3-mercaptopicolinic acid (1.156 g, 7.45 mmol) and HATU (2.83 g, 7.45 mmol) in DMA (30 mL) and the resulting solution stirred at RT for 15 minutes. Dimethylamine (3.11 mL, 6.21 mmol) was added and the reaction was allowed to stir overnight. Water was added to the solution and the solution was extracted with DCM. The organics were washed with a saturated aqueous solution of sodium bicarbonate, dried (MgSO₄) and filtered. Most of the solvent was removed and diethyl ether added. The solid was removed by filtration and discarded. Water was added to the filtrate and the product extracted with ethyl acetate. The organics were dried (MgSO₄), filtered and evaporated to give the desired material (0.52 g).

LCMS Spectrum: m/z (ES−) (M−H)−=181; HPLC tR=1.17 min.

The preparation of 2-chloro-4-(iodomethyl)-6-[(3S)-3-methylmorpholin-4-yl]pyrimidine was described earlier.

### EXAMPLE 51

### 1-[4-[4-[1-(2-Methoxyethylsulfonyl)cyclopropyl]-6-[(3S)-3-methylmorpholin-4-yl]pyrimidin-2-yl]phenyl]-3-methylurea

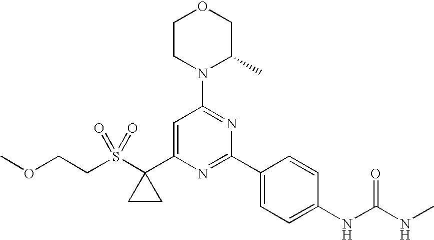

Bis(triphenylphosphine)palladium(II) chloride (5.8 mg, 0.827 mmol) was added to 2-chloro-4-[1-(2-methoxyethylsulfonyl)cyclopropyl]-6-[(3S)-3-methylmorpholin-4-yl]pyrimidine (0.062 g, 0.17 mmol), 1-methyl-3-(4-(4,4,5,5-tetramethyl-1,3,2-dioxaborolan-2-yl)phenyl)urea (0.046 g, 0.17 mmol) and an aqueous solution of sodium carbonate (0.414 mL, 0.83 mmol) in a solvent mixture (2 mL) (the solvent mixture comprised 18% DMF, 82% of a 7:3:2 mixture of DME:water:Ethanol) at RT under an atmosphere of nitrogen. The resulting suspension was stirred at 80° C. for 6 hours. The crude reaction mixture was put down an SCX column, eluting with 7M ammonia in methanol, to give a sample that was concentrated in vacuo and redissolved in DMF (2 mL). The mixture was purified by preparative HPLC to give the desired material as a white solid (17 mg).

NMR Spectrum: ¹H NMR (400.132 MHz, DMSO-d₆) δ 1.22-1.23 (3H, d), 1.54-1.57 (2H, m), 1.64-1.66 (2H, m), 2.65-2.66 (3H, d), 3.16-3.23 (1H, td), 3.27 (3H, s), 3.44-3.51 (1H, td), 3.61-3.64 (1H, dd), 3.75-3.78 (1H, d), 3.80-3.81 (4H, t), 3.95-3.99 (1H, dd), 4.21 (1H, bs), 4.57 (1H, bs), 6.07-6.10 (1H, q), 6.76 (1H, s), 7.49-7.51 (2H, d), 8.17-8.19 (2H, d), 8.75 (1H, s).

LCMS Spectrum: m/z (ES+)(M+H)+=490; HPLC tR=1.93 min.

mTOR Kinase Assay (Echo): 0.00332 μM

The preparation of to 2-chloro-4-[1-(2-methoxyethylsulfonyl)cyclopropyl]-6-[(3S)-3-methylmorpholin-4-yl]pyrimidine is described below.

### 2-Chloro-4-[1-(2-methoxyethylsulfonyl)cyclopropyl]-6-[(3S)-3-methylmorpholin-4-yl]pyrimidine

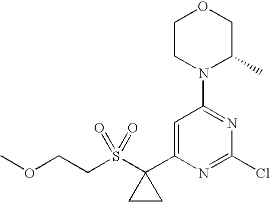

Sodium hydride (0.026 g, 1.07 mmol) was added to (S)-2-(1-(2-chloro-6-(3-methylmorpholino)pyrimidin-4-yl)cyclopropylsulfonyl)ethanol (0.231 g, 0.64 mmol) in THF (20 mL) cooled to 0° C. under a nitrogen atmosphere. The resulting solution was stirred at 0° C. for 10 minutes. To this solution, methyl iodide (0.040 mL, 0.64 mmol) was added and the reaction was slowly allowed to warm to RT. Water was carefully added and the reaction was extracted with DCM. The organics were dried (MgSO₄), filtered and evaporated. The crude product was purified by flash silica chromatography, elution gradient 0 to 60% ethyl acetate in DCM, to give the desired material as a colourless dry film (0.062 g).

NMR Spectrum: ¹H NMR (400.132 MHz, CDCl₃) δ 1.31-1.32 (3H, d), 1.47-1.50 (2H, q), 1.79-1.82 (2H, q), 3.25-3.32 (1H, td), 3.35 (3H, s), 3.44-3.47 (2H, t), 3.49-3.56 (1H, td), 3.65-3.69 (1H, dd), 3.76-3.79 (1H, d), 3.79-3.85 (2H, m), 3.98-4.03 (2H, m), 4.33 (1H, bs), 6.84 (1H, s).

LCMS Spectrum: m/z (ES+)(M+H)+=376; HPLC tR=1.87 min.

### 2-[1-[2-Chloro-6-[(3S)-3-methylmorpholin-4-yl]pyrimidin-4-yl]cyclopropyl]sulfonylethanol

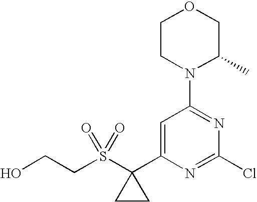

DIPEA (3.70 mL, 21.23 mmol) was added to 2-[1-[2-chloro-6-[(3S)-3-methylmorpholin-4-yl]pyrimidin-4-yl]cyclopropyl]sulfonylacetic acid (3.99 g, 10.62 mmol) in THF cooled to 0° C. under a nitrogen atmosphere. The resulting solution was stirred at 0° C. for 5 minutes then ethyl chloroformate (1.117 mL, 11.68 mmol) added dropwise. The solution was allowed to stir for 1 hour, the solids removed by filtration and the filtrate cooled back to 0° C. Lithium borohydride (17.52 mL, 35.03 mmol) was added and the reaction was slowly allowed to warm to RT. Additional lithium borohydride (10.62 mmol) was added and the mixture stirred for several hours. The reaction was quenched with a saturated aqueous solution of ammonium chloride and then extracted with DCM. The organic layer was separated, washed with saturated brine, dried (MgSO₄), filtered and evaporated. The crude product was purified by flash silica chromatography, elution gradient 0 to 60% ethyl acetate in DCM, to give the desired material as a white solid (1.96 g).

NMR Spectrum: ¹H NMR (400.132 MHz, DMSO-d₆) δ 1.21-1.22(3H, d), 1.49-1.52(2H, m), 1.62-1.65(2H, m), 3.17-3.25(1H, td), 3.40-3.47(1H, td), 3.52-3.55(2H, t), 3.56-3.60(1H, dd), 3.71-3.74(1H, d), 3.79-3.84(2H, q), 3.92-3.95(1H, dd), 4.04(1H, bs), 4.40(1H, bs), 4.98-5.01(1H, t), 6.95(1H, s).

LCMS Spectrum: m/z (ES+)(M+H)+=362; HPLC tR=1.67 min.

### 2-[1-[2-Chloro-6-[(3S)-3-methylmorpholin-4-yl]pyrimidin-4-yl]cyclopropyl]sulfonylacetic acid

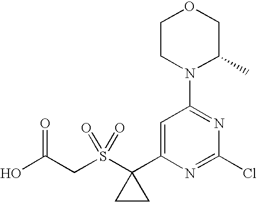

2 M Sodium hydroxide solution (13.85 mL, 27.70 mmol) was added to methyl 2-[1-[2-chloro-6-[(3S)-3-methylmorpholin-4-yl]pyrimidin-4-yl]cyclopropyl]sulfonylacetate (3.60 g, 9.23 mmol) in THF (100 mL) and the resulting solution stirred at RT for 6 hours. The solution was adjusted to pH7 with 2M hydrochloric acid and salt was added to concentrate the solution. The aqueous solution was extracted with DCM, the organic layer separated, dried (MgSO₄), filtered and evaporated to the desired material as a white solid (3.45 g).

NMR Spectrum: ¹H NMR (400.132 MHz, DMSO-d₆) δ 1.20-1.22(3H, d), 1.55-1.58(2H, q), 1.69-1.72(2H, q), 3.18-3.24(1H, m), 3.40-3.46(1H, td), 3.56-3.59(1H, dd), 3.71-3.74(1H, d), 3.92-3.95(1H, dd), 4.04(1H, bs), 4.41(1H, bs), 4.55(2H, s), 6.95(1H, s), 13.36(1H, s).

LCMS Spectrum: m/z (ES+)(M+H)+=376; HPLC tR=0.71 min.

### Methyl 2-[1-[2-chloro-6-[(3S)-3-methylmorpholin-4-yl]pyrimidin-4-yl]cyclopropyl]sulfonylacetate

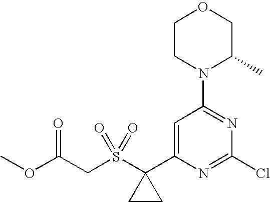

A solution of lithium diisopropylamide (2M solution in THF/n-heptane, 5.46 mL, 9.84 mmol) in THF (60 mL) was added to a stirred solution of 2-chloro-4-[(3S)-3-methylmorpholin-4-yl]-6-(1-methylsulfonylcyclopropyl)pyrimidine (2.720 g, 8.20 mmol), and dimethyl carbonate (6.97 mL, 81.97 mmol) in THF (60 mL), cooled to −78° C., over a period of 5 minutes under an atmosphere of nitrogen. The resulting solution was very slowly allowed to come to RT with stirring over 18 hours. The reaction was cooled back to −78° C. and further lithium diisopropylamide (2.73 mL, 4.92 mmol), and dimethyl carbonate (6.97 mL, 81.97 mmol) added. Again the mixture was allowed to warm slowly to RT with stirring over 24 hours. The reaction mixture was diluted with ethyl acetate (150 mL), washed with 1M citric acid (150 mL) and saturated brine (150 mL). The organic layer was dried (MgSO₄), filtered and evaporated to afford crude product which was purified by flash silica chromatography, elution gradient 10 to 50% ethyl acetate in isohexane, to give the desired material as a yellow gum.

NMR Spectrum: ¹H NMR (400.132 MHz, CDCl₃) δ 1.33 (3H, d), 1.55 (2H, q), 1.91 (2H, q), 3.30 (1H, m), 3.50 (1H, s), 3.68 (1H, q), 3.79 (4H, t), 4.01 (2H, q), 4.28 (2H, s), 4.41 (1H, s), 6.78 (1H, s).

LCMS Spectrum: m/z (ES+)(M+H)+=390; HPLC tR=2.01 min.

The preparation of 2-chloro-4-[(3S)-3-methylmorpholin-4-yl]-6-(1-methylsulfonylcyclopropyl)pyrimidine was described earlier.

### EXAMPLE 52

### 3-cyclopropyl-1-[2-fluoro-4-[4-[(3S)-3-methylmorpholin-4-yl]-6-(1-methylsulfonylcyclopropyl)pyrimidin-2-yl]phenyl]urea

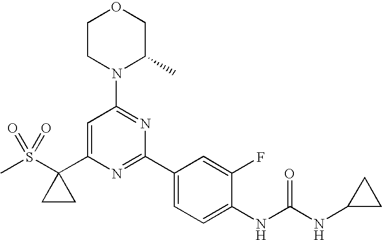

Cyclopropylamine (0.055 mL, 0.80 mmol) followed by triethylamine (0.067 mL, 0.48 mmol) were added to a solution of phenyl N-[2-fluoro-4-[4-[(3S)-3-methylmorpholin-4-yl]-6-(1-methylsulfonylcyclopropyl)pyrimidin-2-yl]phenyl]carbamate (85.4 mg, 0.16 mmol) in DMF (2 mL) and the reaction heated at 50° C. overnight. The crude product was purified by preparative HPLC to give the desired material as a white solid (62 mg).

NMR Spectrum: ¹H NMR (400.132 MHz, DMSO-d₆) δ 0.40-0.44 (2H, m), 0.64-0.69 (2H, m), 1.23-1.25 (3H, d), 1.55-1.58 (2H, q), 1.66-1.69 (2H, q), 2.55-2.61 (1H, m), 3.18-3.25 (1H, td), 3.28 (3H, s), 3.45-3.52 (1H, td), 3.62-3.65 (1H, dd), 3.75-3.78 (1H, d), 3.96-3.99 (1H, dd), 4.21-4.24 (1H, d), 4.58 (1H, bs), 6.81 (1H, s), 6.88-6.89 (1H, d), 8.01-8.04 (1H, dd), 8.06-8.09 (1H, dd), 8.27-8.31 (1H, t), 8.36-8.37 (1H, d).

LCMS Spectrum: m/z (ESI+)(M+H)+490=HPLC tR=2.28 min.

mTOR Kinase Assay (Echo): 0.0184 μM

The compounds below were prepared in an analogous fashion from phenyl N-[2-fluoro-4-[4-[(3S)-3-methylmorpholin-4-yl]-6-(1-methylsulfonylcyclopropyl)pyrimidin-2-yl]phenyl]carbamate and the appropriate amine.

| | | | LCMS | Retention |
|---|---|---|---|---|
| Example | Structure | NAME | MH+ | time (min) |
| 52a | 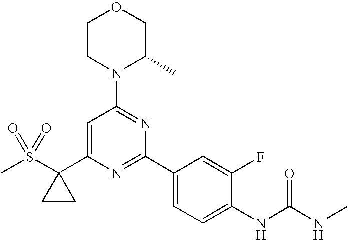 | 1-[2-fluoro-4-[4-[(3S)-3-methylmorpholin-4-yl]-6-(1-methylsulfonylcyclopropyl)pyrimidin-2-yl]phenyl]-3-methylurea | 4.64 | 2.04 |
| 52b | 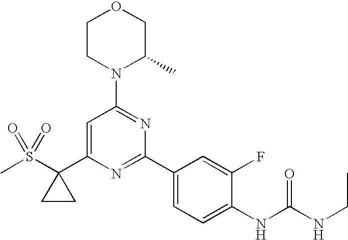 | 3-ethyl-1-[2-fluoro-4-[4-[(3S)-3-methylmorpholin-4-yl]-6-(1-methylsulfonylcyclopropyl)pyrimidin-2-yl]phenyl]urea | 478 | 2.21 |
| 52c | 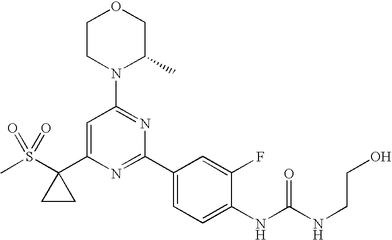 | 1-[2-fluoro-4-[4-[(3S)-3-methylmorpholin-4-yl]-6-(1-methylsulfonylcyclopropyl)pyrimidin-2-yl]phenyl]-3-(2-hydroxyethyl)urea | 4.94 | 1.80 |
| 52d | 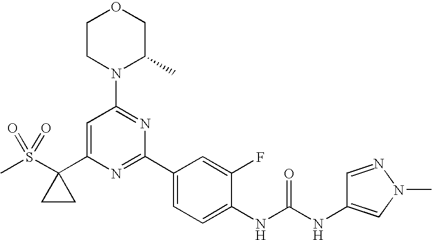 | 1-[2-fluoro-4-[4-[(3S)-3-methylmorpholin-4-yl]-6-(1-methylsulfonylcyclopropyl)pyrimidin-2-yl]phenyl]-3-(1-methylpyrazol-4-yl)urea | 530 | 2.07 |

### EXAMPLE 52a

¹H NMR (400.132 MHz, DMSO-d₆) δ 1.23-1.25 (3H, d), 1.55-1.58 (2H, q), 1.66-1.69 (2H, q), 2.68-2.69 (3H, d), 3.18-3.25 (1H, td), 3.28 (3H, s), 3.45-3.52 (1H, td), 3.62-3.65 (1H, dd), 3.75-3.78 (1H, d), 3.96-3.99 (1H, dd), 4.21-4.24 (1H, d), 4.57 (1H, bs), 6.55-6.58 (1H, q), 6.81 (1H, s), 8.00-8.04 (1H, dd), 8.05-8.08 (1H, dd), 8.26-8.31 (1H, t), 8.54-8.55 (1H, d).

mTOR Kinase Assay (Echo): 0.0103 μM

### EXAMPLE 52b

¹H NMR (400.132 MHz, DMSO-d₆) δ 1.06-1.10 (3H, t), 1.23-1.25 (3H, d), 1.55-1.58 (2H, q), 1.66-1.69 (2H, q), 3.11-3.18 (2H, m), 3.18-3.25 (1H, td), 3.28 (3H, s), 3.45-3.52 (1H, td), 3.62-3.65 (1H, dd), 3.75-3.78 (1H, d), 3.96-3.99 (1H, dd), 4.21-4.24 (1H, d), 4.57 (1H, bs), 6.67-6.69 (1H, t), 6.81 (1H, s), 8.01-8.04 (1H, dd), 8.05-8.08 (1H, dd), 8.27-8.31 (1H, t), 8.47-8.48 (1H, d).

mTOR Kinase Assay (Echo): 0.0307 μM

### EXAMPLE 52c

¹H NMR (400.132 MHz, DMSO-d₆) δ 1.23-1.25 (3H, d), 1.55-1.58 (2H, q) 1.66-1.69 (2H, q), 3.17-3.25 (3H, m), 3.28 (3H, s), 3.45-3.65 (3H, m), 3.62-3.65 (1H, dd), 3.75-3.78 (1H, dd), 3.96-3.99 (1H, dd), 4.21-4.24 (1H, d), 4.58 (1H, bs), 4.73-7.76 (1H, t), 6.81 (1H, s), 6.84-6.87 (1H, t), 8.00-8.04 (1H, dd), 8.05-8.08 (1H, dd), 8.27-8.32 (1H, t), 8.63-8.64 (1H, d).

mTOR Kinase Assay (Echo): 0.0296 μM

### EXAMPLE 52d

¹H NMR (400.132 MHz, DMSO-d₆) δ 1.24-1.25 (3H, d), 1.56-1.59 (2H, q), 1.67-1.70 (2H, q), 3.19-3.26 (1H, td), 3.29 (3H, s), 3.46-3.53 (1H, td), 3.62-3.66 (1H, dd), 3.76-3.79 (1H, d), 3.80 (3H, s), 3.96-3.99 (1H, dd), 4.22-4.25 (1H, d), 4.58 (1H, bs), 6.83 (1H, s), 7.40 (1H, s), 7.79 (1H, s), 8.04-8.08 (1H, dd), 8.09-8.12 (1H, dd), 8.28-8.33 (1H, t), 8.67-8.68 (1H, d), 8.81 (1H, s).

mTOR Kinase Assay (Echo): 0.0107 μM

The preparation of phenyl N-[2-fluoro-4-[4-[(3S)-3-methylmorpholin-4-yl]-6-(1-methylsulfonylcyclopropyl)pyrimidin-2-yl]phenyl]carbamate is described below.

### Phenyl N-[2-fluoro-4-[4-[(3S)-3-methylmorpholin-4-yl]-6-(1-methylsulfonylcyclopropyl)pyrimidin-2-yl]phenyl]carbamate

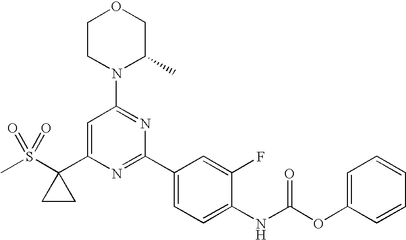

Phenyl chloroformate (0.116 mL, 0.93 mmol) was added to 2-fluoro-4-[4-[(3S)-3-methylmorpholin-4-yl]-6-(1-methylsulfonylcyclopropyl)pyrimidin-2-yl]aniline (0.376 g, 0.93 mmol) and sodium hydrogen carbonate (0.117 g, 1.39 mmol) in dioxane (10 mL) and the resulting solution stirred at RT for 2 hours. Water was added and the solution was extracted with DCM. The organics were dried (MgSO₄), filtered and evaporated. The residue was triturated with diethyl ether to give the desired material as a white solid (0.427 g).

NMR Spectrum: ¹H NMR (400.132 MHz, DMSO-d₆) δ 1.24-1.26 (3H, d), 1.27-1.60 (2H, q), 1.68-1.70 (2H, q), 3.19-3.27 (1H, td), 3.28 (3H, s), 3.46-3.53 (1H, td), 3.62-3.66 (1H, dd), 3.76-3.79 (1H, d), 3.96-4.00 (1H, dd), 4.23-4.26 (1H, d), 4.59 (1H, bs), 6.87 (1H, s), 7.24-7.30 (3H, m), 7.43-7.47 (2H, t), 7.87-7.91 (1H, t), 8.08-8.18 (2H, dd), 10.15 (1H, s).

LCMS Spectrum: m/z (ES+)(M+H)+=527; HPLC tR=2.93 min.

### 2-Fluoro-4-[4-[(3S)-3-methylmorpholin-4-yl]-6-(1-methylsulfonylcyclopropyl)pyrimidin-2-yl]aniline

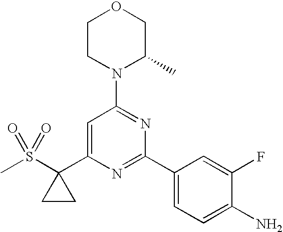

Bis(triphenylphosphine)palladium(II) chloride (0.031 g, 0.04 mmol) was added in one portion to 2-chloro-4-[(3S)-3-methylmorpholin-4-yl]-6-(1-methylsulfonylcyclopropyl)pyrimidine (0.297 g, 0.90 mmol), 2-fluoro-4-(4,4,5,5-tetramethyl-1,3,2-dioxaborolan-2-yl)aniline (0.319 g, 1.34 mmol) and an aqueous solution of sodium carbonate (2.24 mL, 4.48 mmol) in a solvent mixture of 18% DMF, 82% of a 7:3:2 mixture of DME:water:Ethanol. The resulting solution was stirred at 80° C. under a nitrogen atmosphere for 30 minutes. The solvent was removed and the residue partitioned between water and ethyl acetate. The organic layer was dried (MgSO₄), filtered and purified by flash silica chromatography, elution gradient 0 to 30% ethyl acetate in DCM, to give the desired material as a brown gum (0.376 g).

NMR Spectrum: ¹H NMR (400.132 MHz, CDCl₃) δ 1.31-1.33 (3H, d), 1.52-1.55 (2H, q), 1.81-1.84 (2H, q), 3.08 (3H, s), 3.26-3.33 (1H, td), 3.54-3.60 (1H, td), 3.70-3.73 (1H, dd), 3.79-3.80 (1H, d), 4.00-4.03 (3H, m), 4.13 (1H, bs), 4.46-4.47 (1H, bs), 6.72 (1H, s), 6.76-6.80 (1H, t), 7.99-8.02 (2H, m).

LCMS Spectrum: m/z (ES+)(M+H)+=407; HPLC tR=2.29 min.

The preparation of 2-chloro-4-[(3S)-3-methylmorpholin-4-yl]-6-(1-methylsulfonylcyclopropyl)pyrimidine was described earlier.

### EXAMPLE 53

### 1-[4-[4-[1-(3-Fluorophenyl)sulfonylcyclopropyl]-6-[(3S)-3-methylmorpholin-4-yl]pyrimidin-2-yl]phenyl]-3-methylurea

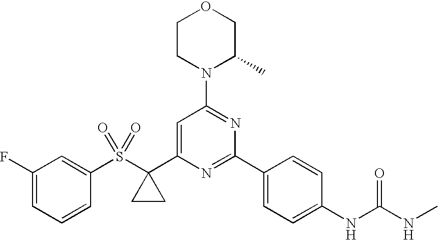

Methylamine (26.4 mg, 0.85 mmol) was added to phenyl N-[4-[4-[1-(3-fluorophenyl)sulfonylcyclopropyl]-6-[(3S)-3-methylmorpholin-4-yl]pyrimidin-2-yl]phenyl]carbamate (100 mg, 0.17 mmol) in NMP (2 mL). The resulting solution was heated at 50° C. for 2 days. The crude product was purified by preparative HPLC, using decreasingly polar mixtures of water (containing 1% NH₃) and MeCN as eluents, to give the desired material as a white solid (41 mg).

NMR Spectrum: ¹H NMR (399.902 MHz, DMSO-d₆) δ 1.19 (3H, d), 1.65 (2H, m), 1.94 (2H, m), 2.67 (4H, m), 3.46 (1H, m), 3.61 (1H, m), 3.75 (1H, m), 3.96 (1H, m), 4.14 (1H, m), 4.44 (1H, m), 6.05 (1H, m), 6.65 (1H, s), 7.40 (2H, m), 7.63 (4H, m), 7.82 (2H, m), 8.72 (1H, m)

LCMS Spectrum: m/z (ESI+)(M+H)+=526; HPLC tR=2.07 min.

The compounds below were prepared in an analogous fashion from phenyl N-[4-[4-[1-(3-fluorophenyl)sulfonylcyclopropyl]-6-[(3S)-3-methylmorpholin-4-yl]pyrimidin-2-yl]phenyl]carbamate and the appropriate amine.

| | | | | Retention |
|---|---|---|---|---|
| Ex- | | | LCMS | time |
| ample | Structure | NAME | MH+ | (min) |
| 53a | 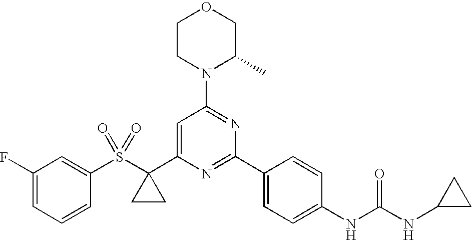 | 3-cyclopropyl-1-[4-[4-[1-(3-fluorophenyl)sulfonylcyclopro-pyl]-6-[(3S)-3-methylmorpholin-4-yl]pyrimidin-2-yl]phenyl]urea | 552 | 2.28 |
| 53b | 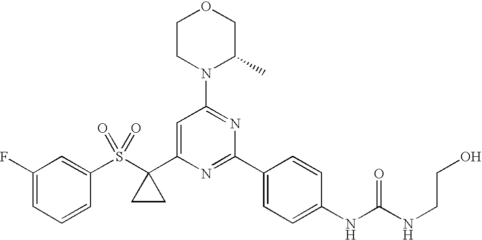 | 1-[4-[4-[1-(3-fluorophenyl)sulfonylcyclopro-pyl]-6-[(3S)-3-methylmorpholin-4-yl]pyrimidin-2-yl]phenyl]-3-(2-hydroxyethyl)urea | 556 | 1.97 |
| 53c | 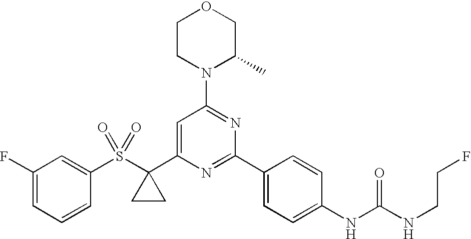 | 3-(2-fluoroethyl)-1-[4-[4-[1-(3-fluorophenyl)sulfonylcyclopro-pyl]-6-[(3S)-3-methylmorpholin-4-yl]pyrimidin-2-yl]phenyl]urea | 558 | 2.27 |
| 53d | 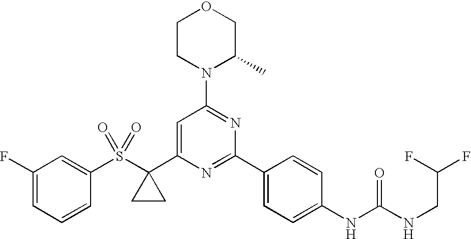 | 3-(2,2-difluoroethyl)-1-[4-[4-[1-(3-fluorophenyl)sulfonylcyclopro-pyl]-6-[(3S)-3-methylmorpholin-4-yl]pyrimidin-2-yl]phenyl]urea | 576 | 2.39 |
| 53e | 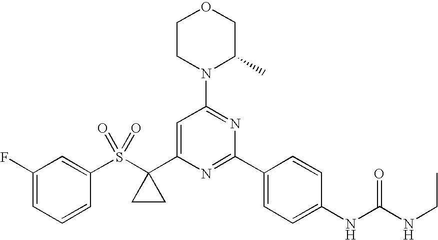 | 1-ethyl-3-[4-[4-[1-(3-fluorophenyl)sulfonylcyclopro-pyl]-6-[(3S)-3-methylmorpholin-4-yl]pyrimidin-2-yl]phenyl]urea | 540 | 2.27 |
| 53f | 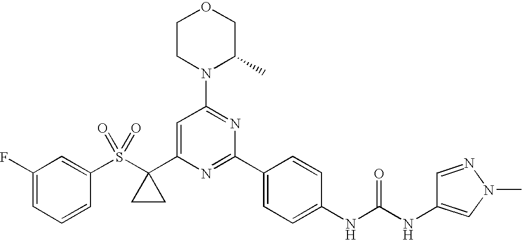 | 1-[4-[4-[1-(3-fluorophenyl)sulfonylcyclopro-pyl]-6-[(3S)-3-methylmorpholin-4-yl]pyrimidin-2-yl]phenyl]-3-(1-methylpyrazol-4-yl)urea | 592 | 2.20 |

### EXAMPLE 53a

¹H NMR (399.902 MHz, DMSO-d₆) δ 0.43 (2H, m), 0.66 (2H, m), 1.19 (3H, d), 1.65 (2H, m), 1.94 (2H, m), 2.56 (1H, m), 3.16 (1H, m), 3.46 (1H, m), 3.61 (1H, m), 3.75 (1H, m), 3.96 (1H, m), 4.14 (1H, m), 4.44 (1H, m), 6.41 (1H, m), 6.65 (1H, s), 7.40 (2H, m), 7.62 (4H, m), 7.82 (2H, m), 8.52 (1H, s)

### EXAMPLE 53b

¹H NMR (399.902 MHz, DMSO-d₆) δ 1.19 (3H, d), 1.65 (2H, m), 1.93 (2H, m), 3.18 (3H, m), 3.46 (3H, m), 3.62 (1H, m), 3.75 (1H, m), 3.96 (1H, m), 4.15 (1H, m), 4.43 (1H, m), 4.74 (1H, m), 6.23 (1H, m), 6.65 (1H, s), 7.39 (2H, m), 7.63 (4H, m), 7.83 (2H, m), 8.78 (1H, s)

### EXAMPLE 53c

¹H NMR (399.902 MHz, DMSO-d₆) δ 1.19 (3H, d), 1.65 (2H, m), 1.93 (2H, m), 3.16 (1H, m), 3.43 (3H, m), 3.62 (1H, m), 3.75 (1H, m), 3.96 (1H, m), 4.16 (1H, m), 4.43 (2H, m), 4.54 (1H, m), 6.42 (1H, m), 6.66 (1H, s), 7.40 (2H, m), 7.63 (4H, m), 7.83 (2H, m), 8.79 (1H, s)

### EXAMPLE 53d

¹H NMR (399.902 MHz, DMSO-d₆) δ 1.19 (3H, d), 1.65 (2H, m), 1.94 (2H, m), 3.16 (1H, m), 3.53 (4H, m), 3.75 (1H, m), 3.96 (1H, m), 4.15 (1H, m), 4.45 (1H, m), 6.07 (1H, m), 6.53 (1H, m), 6.66 (1H, s), 7.41 (2H, m), 7.63 (4H, m), 7.84 (2H, m), 8.91 (1H, s)

### EXAMPLE 53e

¹H NMR (399.902 MHz, DMSO-d₆) δ 1.07 (3H, t), 1.19 (3H, d), 1.65 (2H, m), 1.94 (2H, m), 3.13 (3H, m), 3.47 (1H, m), 3.62 (1H, m), 3.74 (1H, m), 3.96 (1H, m), 4.13 (1H, m), 4.44 (1H, m), 6.14 (1H, m), 6.65 (1H, s), 7.39 (2H, m), 7.63 (4H, m), 7.82 (2H, m), 8.64 (1H, s)

### EXAMPLE 53f

¹H NMR (399.902 MHz, DMSO-d₆) δ 1.20 (3H, d), 1.65 (2H, m), 1.95 (2H, m), 3.17 (1H, m), 3.47 (1H, m), 3.62 (1H, m), 3.78 (4H, m), 3.97 (1H, m), 4.15 (1H, m), 4.45 (1H, m), 6.66 (1H, s), 7.42 (3H, m), 7.65 (4H, m), 7.77 (1H, s), 7.86 (2H, d), 8.38 (1H, s), 8.81 (1H, s)

The preparation of phenyl N-[4-[4-[1-(3-fluorophenyl)sulfonylcyclopropyl]-6-[(3S)-3-methylmorpholin-4-yl]pyrimidin-2-yl]phenyl]carbamateis described below:

### Phenyl N-[4-[4-[1-(3-fluorophenyl)sulfonylcyclopropyl]-6-[(3S)-3-methylmorpholin-4-yl]pyrimidin-2-yl]phenyl]carbamate

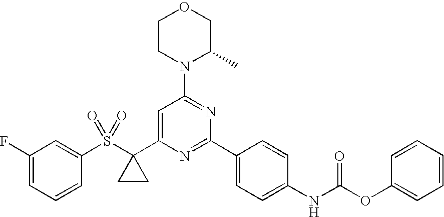

Phenyl chloroformate (0.206 mL, 1.64 mmol) was added dropwise to 4-[4-[1-(3-fluorophenyl)sulfonylcyclopropyl]-6-[(3S)-3-methylmorpholin-4-yl]pyrimidin-2-yl]aniline (770 mg, 1.64 mmol) and sodium bicarbonate (138 mg, 1.64 mmol) in dioxane (30 mL). The resulting solution was stirred at RT for 3 hours. The reaction mixture was filtered and the precipitate collected and redissolved in DCM (100 mL). This was washed sequentially with water (100 mL) and saturated brine (100 mL). The organic layer was dried over MgSO₄, filtered and evaporated to afford the desired material as a yellow gum (722 mg).

NMR Spectrum: ¹H NMR (399.902 MHz, DMSO-d₆) δ 1.19 (3H, d), 1.65 (2H, m), 1.95 (2H, m), 3.17 (1H, m), 3.46 (1H, m), 3.61 (1H, m), 3.75 (1H, m), 3.96 (1H, m), 4.16 (1H, m), 4.46 (1H, m), 6.70 (1H, s), 7.27 (3H, m), 7.55 (8H, m), 7.92 (2H, m), 10.40 (1H, s)

LCMS Spectrum: m/z (ESI+)(M+H)+=589; HPLC tR=3.02 min.

### 4-[4-[1-(3-Fluorophenyl)sulfonylcyclopropyl]-6-[(3S)-3-methylmorpholin-4-yl]pyrimidin-2-yl]aniline

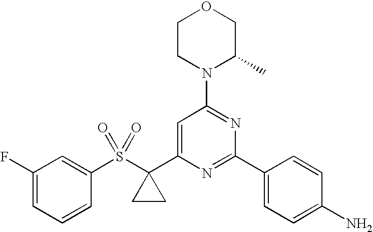

1,1′-Bis(diphenylphosphino)ferrocenedichloropalladium(II) (0.747 g, 1.03 mmol) was added to 2-chloro-4-[1-(3-fluorophenyl)sulfonylcyclopropyl]-6-[(3S)-3-methylmorpholin-4-yl]pyrimidine (4.25 g, 10.32 mmol) and 4-(4,4,5,5-tetramethyl-1,3,2-dioxaborolan-2-yl)aniline (2.487 g, 11.35 mmol) in DME (200 mL) and sodium carbonate (5.47 g, 51.59 mmol) in water (25 mL) under nitrogen. The resulting solution was stirred at 80° C. for 5 hours. The reaction mixture was diluted with DCM (200 mL), and washed sequentially with water (200 mL) and saturated brine (200 mL). The organic layer was dried over MgSO₄, filtered and evaporated to afford crude product. The crude product was purified by flash silica chromatography, elution gradient 0 to 20% ethyl acetate in DCM, to afford crude product as a yellow gum. The crude product was further purified by flash silica chromatography, elution gradient 0 to 5% ethyl acetate in DCM, to give the desired material as a yellow gum (2.86 g).

NMR Spectrum: ¹H NMR (399.902 MHz, DMSO-d₆) δ 1.17 (3H, d), 1.63 (2H, m), 1.92 (2H, m), 3.12 (1H, m), 3.45 (1H, m), 3.60 (1H, m), 3.74 (1H, m), 3.95 (1H, m), 4.11 (1H, m) 4.40 (1H, s), 5.52 (2H, s), 6.50 (2H, m), 6.57 (1H, m), 7.63 (6H, m)

LCMS Spectrum: m/z (ESI+)(M+H)+=469; HPLC tR=2.35 min.

### 2-Chloro-4-[1-(3-fluorophenyl)sulfonylcyclopropyl]-6-[(3S)-3-methylmorpholin-4-yl]pyrimidine

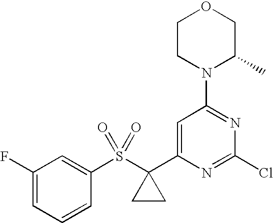

Sodium hydroxide (24.80 g, 620.07 mmol) in water (24.8 mL) was added to 2-chloro-4-[(3-fluorophenyl)sulfonylmethyl]-6-[(3S)-3-methylmorpholin-4-yl]pyrimidine (4.35 g, 11.27 mmol), 1,2-dibromoethane (2.91 mL, 33.82 mmol) and tetrabutylammonium bromide (0.363 g, 1.13 mmol) in toluene (200 mL). The resulting solution was stirred at 60° C. for 3 hours. The reaction mixture was diluted with DCM (200 mL), and washed twice with water (200 mL). The organic layer was dried over MgSO₄, filtered and evaporated to afford crude product. The crude product was purified by flash silica chromatography, elution gradient 0 to 20% ethyl acetate in DCM, to give the desired material as a white solid (4.25 g).

NMR Spectrum: ¹H NMR (399.902 MHz, DMSO-d₆) δ 1.20 (3H, d), 1.63 (2H, m), 1.94 (2H, m), 3.18 (1H, m), 3.45 (1H, m), 3.60 (1H, m), 3.74 (1H, m), 3.97 (2H, m), 4.32 (1H, m), 6.76 (1H, s), 7.69 (4H, m)

LCMS Spectrum: m/z (ESI+)(M+H)+=412; HPLC tR=2.27 min.

### 2-Chloro-4-[(3-fluorophenyl)sulfonylmethyl]-6-[(3S)-3-methylmorpholin-4-yl]pyrimidine

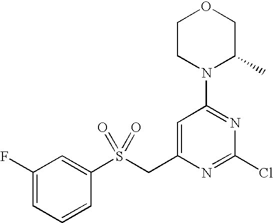

2-Chloro-4-(iodomethyl)-6-[(3S)-3-methylmorpholin-4-yl]pyrimidine (7 g, 19.80 mmol) was added to sodium 3-fluorobenzenesulfinate (5.41 g, 29.70 mmol) in acetonitrile (100 mL) under nitrogen. The resulting suspension was stirred at 80° C. for 3 hours. The reaction mixture was evaporated to dryness and redissolved in DCM (200 mL), and washed sequentially with water (200 mL) and saturated brine (200 mL). The organic layer was dried over MgSO4, filtered and evaporated to afford crude product. The crude product was purified by flash silica chromatography, elution gradient 0 to 5% ethyl acetate in DCM, to give the desired material as a white solid (6.38 g).

NMR Spectrum: ¹H NMR (399.902 MHz, DMSO-d₆) δ 1.17 (3H, d), 3.16 (1H, m), 3.43 (1H, m), 3.58 (1H, m), 3.73 (1H, m), 3.93 (2H, m), 4.18 (1H, m), 4.71 (2H, s), 6.74 (1H, s), 7.68 (4H, m)

LCMS Spectrum: m/z (ESI+)(M+H)+=386; HPLC tR=2.06 min.

The preparation of 2-chloro-4-(iodomethyl)-6-[(3S)-3-methylmorpholin-4-yl]pyrimidine was described earlier.

### Sodium 3-fluorobenzenesulfinate

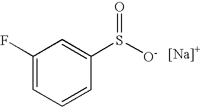

A solution of sodium sulphite (7.77 g, 61.66 mmol) in water (60 mL) was stirred at RT for 10 minutes. Sodium bicarbonate (10.36 g, 123.32 mmol) was added and the resulting solution was stirred at 50° C. for 1 hour. 3-Fluorobenzene-1-sulfonyl chloride (8.20 mL, 61.66 mmol) was added dropwise and the resulting solution was stirred at 50° C. for 20 hours. The reaction mixture was evaporated to dryness and redissolved in MeOH. The suspension was allowed to stir at RT for 20 minutes. The suspension was filtered and the filtrate evaporated to afford the desired material (12.60 g) as a white solid, which was air dried overnight under vacuum and used without further purification.

NMR Spectrum: ¹H NMR (399.902 MHz, DMSO-d₆) δ 7.03 (1H, m), 7.21 (1H, m), 7.29 (1H, m), 7.36 (1H, m)

### EXAMPLE 54

### 3-Methyl-1-[4-[4-[(3S)-3-methylmorpholin-4-yl]-6-[1-(2-methylphenyl)sulfonylcyclopropyl]pyrimidin-2-yl]phenyl]urea

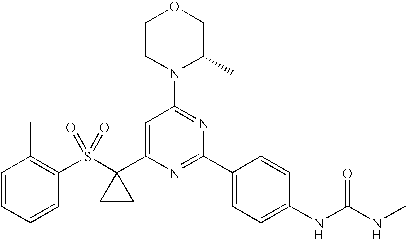

Methylamine (37.27 mg, 1.2 mmol) was added to phenyl N-[4-[4-[(3S)-3-methylmorpholin-4-yl]-6-[1-(2-methylphenyl)sulfonylcyclopropyl]pyrimidin-2-yl]phenyl]carbamate (140 mg, 0.24 mmol) and triethylamine (0.2 mL1, 0.72 mmol) in NMP (2 mL). The resulting solution was heated at 50° C. for 2 days. The crude product was purified by preparative HPLC, using decreasingly polar mixtures of water (containing 1% NH3) and MeCN as eluents, to give the desired material (87 mg).

NMR Spectrum: ¹H NMR (399.902 MHz, DMSO-d₆) δ 1.15 (3H, d), 1.66 (2H, m), 1.86 (2H, m), 2.45 (3H, s), 2.66 (3H, m), 3.12 (1H, m), 3.45 (1H, m), 3.60 (1H, m), 3.74 (1H, m), 3.94 (1H, m), 4.06 (1H, m), 4.39 (1H, m), 6.05 (1H, m), 6.60 (1H, s), 7.37 (4H, m), 7.51 (1H, m), 7.85 (3H, m), 8.71 (1H, s)

LCMS Spectrum: m/z (ESI+)(M+H)+=522; HPLC tR=2.29 min. The compounds below were prepared in an analogous fashion from phenyl N-[4-[4-[(3S)-3-methylmorpholin-4-yl]-6-[1-(2-methylphenyl)sulfonylcyclopropyl]pyrimidin-2-yl]phenyl]carbamate and the appropriate amine.

| | | | | Retention |
|---|---|---|---|---|
| | | | LCMS | time |
| Example | Structure | NAME | MH+ | (min) |
| 54a | 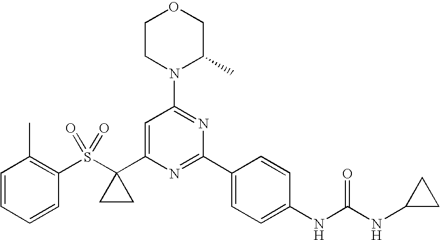 | 3-cyclopropyl-1-[4-[4-[(3S)-3-methylmorpholin-4-yl]-6-[1-(2-methylphenyl)sulfonylcyclo-propyl]pyrimidin-2-yl]phenyl]urea | 548 | 2.47 |
| 54b | 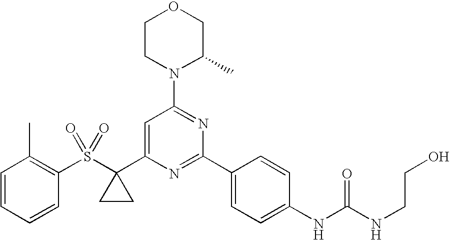 | 3-(2-hydroxyethyl)-1-[4-[4-[(3S)-3-methylmorpholin-4-yl]-6-[1-(2-methylphenyl)sulfonylcyclo-propyl]pyrimidin-2-yl]phenyl]urea | 552 | 2.09 |
| 54c | 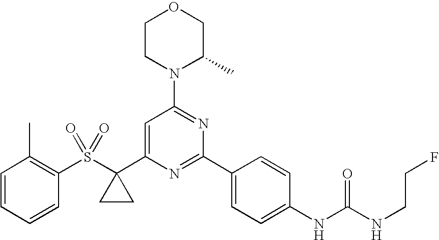 | 3-(2-fluoroethyl)-1-[4-[4-[(3S)-3-methylmorpholin-4-yl]-6-[1-(2-methylphenyl)sulfonylcyclo-propyl]pyrimidin-2-yl]phenyl]urea | 554 | 2.42 |
| 54d | 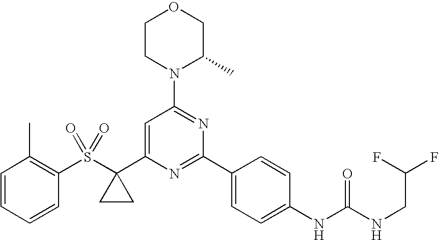 | 3-(2,2-difluoroethyl)-1-[4-[4-[(3S)-3-methylmorpholin-4-yl]-6-[1-(2-methylphenyl)sulfonylcyclo-propyl]pyrimidin-2-yl]phenyl]urea | 572 | 2.56 |
| 54e | 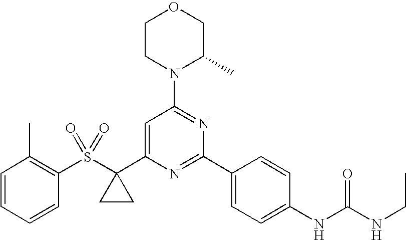 | 1-ethyl-3-[4-[4-[(3S)-3-methylmorpholin-4-yl]-6-[1-(2-methylphenyl)sulfonylcyclo-propyl]pyrimidin-2-yl]phenyl]urea | 536 | 2.45 |
| 54f | 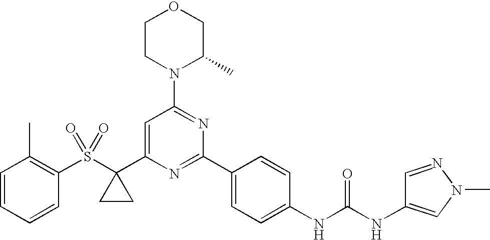 | 1-[4-[4-[(3S)-3-methylmorpholin-4-yl]-6-[1-(2-methylphenyl)sulfonylcyclo-propyl]pyrimidin-2-yl]phenyl]-3-(1-methylpyrazol-4-yl)urea | 588 | 2.31 |

### EXAMPLE 54a

¹H NMR (399.902 MHz, DMSO-d₆) δ 0.42 (2H, m), 0.65 (2H, m), 1.15 (3H, d), 1.66 (2H, m), 1.86 (2H, m), 2.46 (3H, s), 2.56 (1H, m), 3.13 (1H, m), 3.44 (1H, m), 3.59 (1H, m), 3.74 (1H, m), 3.95 (1H, m), 4.07 (1H, m), 4.39 (1H, m), 6.42 (1H, m), 6.60 (1H, s), 7.37 (4H, m), 7.51 (1H, m), 7.84 (3H, m), 8.51 (1H, s)

### EXAMPLE 54b

¹H NMR (399.902 MHz, DMSO-d₆) δ 1.15 (3H, d), 1.66 (2H, m), 1.86 (2H, m), 2.46 (3H, s), 3.14 (3H, m), 3.45 (3H, m), 3.59 (1H, m), 3.74 (1H, m), 3.95 (1H, m), 4.07 (1H, m), 4.38 (1H, m), 4.74 (1H, t), 6.24 (1H, m), 6.60 (1H, s), 7.36 (4H, m), 7.51 (1H, m), 7.84 (3H, m), 8.77 (1H, s)

### EXAMPLE 54c

¹H NMR (399.902 MHz, DMSO-d₆) δ 1.15 (3H, d), 1.66 (2H, m), 1.86 (2H, m), 2.45 (3H, s), 3.12 (1H, m), 3.43 (3H, m), 3.60 (1H, m), 3.74 (1H, m), 3.95 (1H, m), 4.07 (1H, m), 4.40 (2H, m), 4.54 (1H, m), 6.42 (1H, m), 6.61 (1H, s), 7.37 (4H, m), 7.51 (1H, m), 7.85 (3H, m), 8.78 (1H, s)

### EXAMPLE 54d

¹H NMR (399.902 MHz, DMSO-d₆) δ 1.15 (3H, d), 1.66 (2H, m), 1.86 (2H, m), 2.46 (3H, s), 3.11 (1H, m), 3.51 (4H, m), 3.75 (1H, m), 3.95 (1H, m), 4.07 (1H, m), 4.39 (1H, m), 6.08 (1H, m), 6.52 (1H, m), 6.61 (1H, s), 7.38 (4H, m), 7.51 (1H, m), 7.85 (3H, m), 8.89 (1H, s)

### EXAMPLE 54e

¹H NMR (399.902 MHz, DMSO-d₆) δ 1.07 (3H, t), 1.15 (3H, d), 1.66 (2H, m), 1.85 (2H, m), 2.45 (3H, s), 3.12 (3H, m), 3.44 (1H, m), 3.60 (1H, m), 3.74 (1H, m), 3.95 (1H, m), 4.06 (1H, m), 4.39 (1H, m), 6.15 (1H, m), 6.60 (1H, s), 7.37 (4H, m), 7.51 (1H, m), 7.84 (3H, m), 8.63 (1H, s)

### EXAMPLE 54f

¹H NMR (399.902 MHz, DMSO-d₆) δ 1.16 (3H, d), 1.66 (2H, m), 1.86 (2H, m), 2.46 (3H, s), 3.12 (1H, m), 3.45 (1H, m), 3.60 (1H, m), 3.74 (1H, m), 3.80 (3H, s), 3.95 (1H, m), 4.07 (1H, m), 4.39 (1H, m), 6.61 (1H, s), 7.43 (6H, m), 7.80 (2H, m), 7.90 (2H, m), 8.37 (1H, s), 8.80 (1H, s)

The preparation of phenyl N-[4-[4-[(3S)-3-methylmorpholin-4-yl]-6-[1-(2-methylphenyl)sulfonylcyclopropyl]pyrimidin-2-yl]phenyl]carbamate is described below:

### Phenyl N-[4-[4-[(3S)-3-methylmorpholin-4-yl]-6-[1-(2-methylphenyl)sulfonylcyclopropyl]pyrimidin-2-yl]phenyl]carbamate

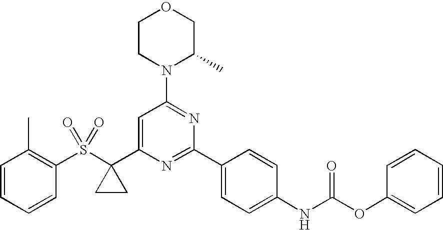

Phenyl chloroformate (0.205 mL, 1.64 mmol) was added dropwise to 4-[4-[(3S)-3-methylmorpholin-4-yl]-6-[1-(2-methylphenyl)sulfonylcyclopropyl]pyrimidin-2-yl]aniline (760 mg, 1.64 mmol) and sodium bicarbonate (151 mg, 1.80 mmol) in dioxane (30 mL). The resulting suspension was stirred at RT for 3 hours. The reaction mixture was filtered and the precipitate was redissolved in DCM (50 mL), and washed sequentially with water (50 mL) and saturated brine (50 mL). The organic layer was dried over MgSO₄, filtered and evaporated to afford the desired material as a yellow gum (985 mg).

NMR Spectrum: ¹H NMR (399.902 MHz, DMSO-d₆) δ 1.16 (3H, d), 1.67 (2H, m), 1.87 (2H, m), 2.47 (3H, s), 3.12 (1H, m), 3.45 (1H, m), 3.61 (1H, m), 3.74 (1H, m), 3.95 (1H, m), 4.08 (1H, m), 4.40 (1H, m), 6.64 (1H, s), 7.30 (5H, m), 7.49 (5H, m), 7.81 (1H, m), 7.95 (2H, m), 10.39 (1H, s)

LCMS Spectrum: m/z (ESI+)(M+H)+=585; HPLC tR=3.08 min.

### 4-[4-[(3S)-3-Methylmorpholin-4-yl]-6-[1-(2-methylphenyl)sulfonylcyclopropyl]pyrimidin-2-yl]aniline

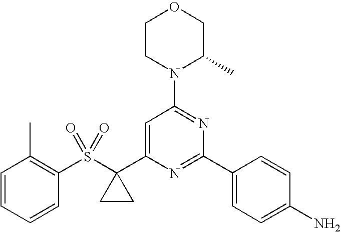

A solution of 2-chloro-4-[(3S)-3-methylmorpholin-4-yl]-6-[1-(2-methylphenyl)sulfonylcyclopropyl]pyrimidine (2.04 g, 5.00 mmol), 4-(4,4,5,5-tetramethyl-1,3,2-dioxaborolan-2-yl)aniline (1.205 g, 5.50 mmol) and sodium carbonate (2.120 g, 20.00 mmol) in DME (60 mL) and water (15.00 mL) was stirred under nitrogen for 5 minutes. Dichlorobis(triphenylphosphine)palladium(II) (0.351 g, 0.50 mmol) was added and the resulting solution was stirred at 80° C. for 2 hours. The reaction mixture was diluted with DCM (200 mL), and washed with water (200 mL). The organic layer was dried over MgSO₄, filtered and evaporated to afford crude product. The crude product was purified by flash silica chromatography, elution gradient 20 to 50% ethyl acetate in isohexane, to give the desired material as a yellow oil which solidified on standing (2.14 g).

NMR Spectrum: ¹H NMR (399.902 MHz, DMSO-d₆) δ 1.18 (3H, d), 1.69 (2H, m), 1.87 (2H, m), 2.51 (3H, s), 3.12 (1H, m), 3.47 (1H, m), 3.63 (1H, m), 3.77 (1H, m), 3.98 (1H, m), 4.08 (1H, m), 4.39 (1H, m), 5.56 (2H, s), 6.54 (3H, m), 7.39 (2H, m), 7.56 (1H, m), 7.77 (2H, m), 7.87 (1H, m)

LCMS Spectrum: m/z (ESI+)(M+H)+=465; HPLC tR=2.41 min.

### 2-Chloro-4-[(3S)-3-methylmorpholin-4-yl]-6-[1-(2-methylphenyl)sulfonylcyclopropyl]pyrimidine

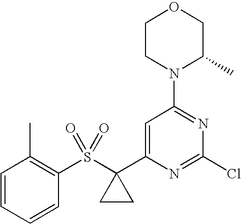

Sodium hydroxide (12.67 g, 317 mmol) in water (12.7 mL) was added to 2-chloro-4-[(3S)-3-methylmorpholin-4-yl]-6-[(2-methylphenyl)sulfonylmethyl]pyrimidine (2.2 g, 5.76 mmol), 1,2-dibromoethane (1.489 mL, 17.28 mmol) and tetrabutylammonium bromide (0.186 g, 0.58 mmol) in toluene (80 mL). The resulting solution was stirred at 60° C. for 1 hour. The reaction mixture was diluted with DCM (200 mL), and washed sequentially with water (200 mL) and saturated brine (200 mL). The organic layer was dried over MgSO₄, filtered and evaporated to afford crude product. The crude product was purified by flash silica chromatography, elution gradient 0 to 40% ethyl acetate in DCM, to give the desired material as a colourless gum which solidified on standing (2.04 g).

NMR Spectrum: ¹H NMR (399.902 MHz, DMSO-d₆) δ 1.17 (3H, d), 1.63 (2H, m), 1.86 (2H, m), 2.48 (3H, s), 3.14 (1H, m), 3.42 (1H, m), 3.58 (1H, m), 3.74 (1H, m), 3.95 (2H, m), 4.25 (1H, m), 6.70 (1H, s), 7.43 (2H, m), 7.63 (1H, m), 7.79 (1H, m)

LCMS Spectrum: m/z (ESI+)(M+H)+=408; HPLC tR=2.44 min.

### 2-Chloro-4-[(3S)-3-methylmorpholin-4-yl]-6-[(2-methylphenyl)sulfonylmethyl]pyrimidin

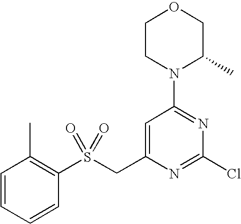

2N sulfuric acid (0.352 mL) was added to 2-chloro-4-[(3S)-3-methylmorpholin-4-yl]-6-[(2-methylphenyl)sulfanylmethyl]pyrimidine (4.37 g, 12.49 mmol) in dioxane (110 mL) and the solution heated to 55° C. Sodium tungstate dihydrate (0.082 g, 0.25 mmol) in water (3.5 mL) was added and the solution was allowed to stir for 5 minutes. Hydrogen peroxide (7.65 mL, 74.94 mmol) was added dropwise to the solution. The resulting solution was stirred at 55° C. for 5 hours. The reaction was cooled to RT then water added until precipitation ceased. The precipitate was collected by filtration, washed with water and dried under vacuum to afford the desired material as a white solid (3.70 g).

NMR Spectrum: ¹H NMR (399.902 MHz, DMSO-d₆) δ 1.16 (3H, d), 2.63 (3H, s), 3.14 (1H, m), 3.42 (1H, m), 3.56 (1H, m), 3.72 (1H, m), 3.93 (2H, m), 4.18 (1H, s), 4.59 (2H, s), 6.67 (1H, s), 7.44 (2H, m), 7.65 (2H, m)

LCMS Spectrum: m/z (ESI+)(M+H)+=382; HPLC tR=2.23 min.

### 2-Chloro-4-[(3S)-3-methylmorpholin-4-yl]-6-[(2-methylphenyl)sulfanylmethyl]pyrimidine

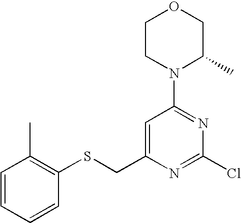

DIPEA (3.70 mL, 21.21 mmol) was added to 2-chloro-4-(iodomethyl)-6-[(3S)-3-methylmorpholin-4-yl]pyrimidine (5 g, 14.14 mmol) and 2-methylbenzenethiol (2.5 mL, 21.21 mmol) in THF (80 mL). The resulting slurry was stirred at RT for 18 hours. The reaction mixture was evaporated to dryness and redissolved in DCM (100 mL), and washed sequentially with water (100 mL) and saturated brine (100 mL). The organic layer was dried over MgSO₄, filtered and evaporated to afford crude product. The crude product was purified by flash silica chromatography, elution gradient 0 to 5% ethyl acetate in isohexane, to give the desired material as a yellow gum (4.37 g).

NMR Spectrum: ¹H NMR (399.902 MHz, DMSO-d₆) δ 1.13 (3H, d), 2.28 (3H, s), 3.11 (1H, m), 3.40 (1H, m), 3.55 (1H, m), 3.69 (1H, m), 3.90 (2H, m), 4.04 (2H, m), 4.21 (1H, m), 6.65 (1H, s), 7.18 (3H, m), 7.37 (1H, m)

LCMS Spectrum: m/z (ESI+)(M+H)+=350; HPLC tR=2.71 min.

The preparation of 2-chloro-4-(iodomethyl)-6-[(3S)-3-methylmorpholin-4-yl]pyrimidine was described earlier.

### EXAMPLE 55

### 1-[4-[4-[1-(1,3-Dimethylpyrazol-4-yl)sulfonylcyclopropyl]-6-[(3S)-3-methylmorpholin-4-yl]pyrimidin-2-yl]phenyl]-3-methylurea

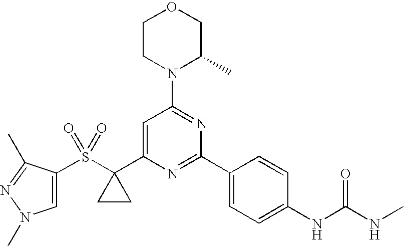

Methylamine (26.4 mg, 0.85 mmol) was added to phenyl N-[4-[4-[1-(1,3-dimethylpyrazol-4-yl)sulfonylcyclopropyl]-6-[(3S)-3-methylmorpholin-4-yl]pyrimidin-2-yl]phenyl]carbamate (100 mg, 0.17 mmol) and triethylamine (0.2 mL, 0.72 mmol) in NMP (2 mL). The resulting solution was heated at 50° C. for 2 days. The crude product was purified by preparative HPLC, using decreasingly polar mixtures of water (containing 1% NH3) and MeCN as eluents, to give the desired material (77 mg).

NMR Spectrum: ¹H NMR (399.902 MHz, DMSO-d₆) δ 1.20 (3H, d), 1.54 (2H, m), 1.74 (2H, m), 2.03 (3H, s), 2.67 (3H, m), 3.20 (1H, m), 3.48 (1H, m), 3.63 (1H, m), 3.77 (4H, m), 3.98 (1H, m), 4.13 (1H, m), 4.46 (1H, m), 6.06 (1H, m), 6.70 (1H, s), 7.45 (2H, d), 7.98 (2H, d), 8.18 (1H, s), 8.72 (1H, s)

LCMS Spectrum: m/z (ESI+)(M+H)+=526; HPLC tR=1.87 min.

The compounds below were prepared in an analogous fashion from phenyl N-[4-[4-[1-(1,3-dimethylpyrazol-4-yl)sulfonylcyclopropyl]-6-[(3S)-3-methylmorpholin-4-yl]pyrimidin-2-yl]phenyl]carbamate and the appropriate amine.

| | | | | Retention |
|---|---|---|---|---|
| | | | LCMS | time |
| Example | Structure | NAME | MH+ | (min) |
| 55a | 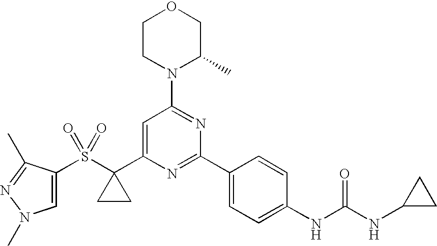 | 3-cyclopropyl-1-[4-[4-[1-(1,3-dimethylpyrazol-4-yl)sulfonylcyclopropyl]-6-[(3S)-3-methylmorpholin-4-yl]pyrimidin-2-yl]phenyl]urea | 552 | 2.02 |
| 55b | 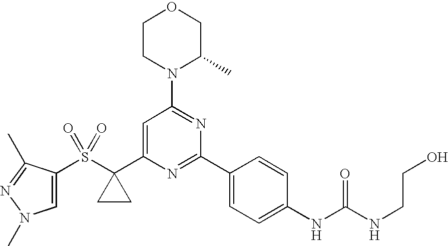 | 1-[4-[4-[1-(1,3-dimethylpyrazol-4-yl)sulfonylcyclopropyl]-6-[(3S)-3-methylmorpholin-4-yl]pyrimidin-2-yl]phenyl]-3-(2-hydroxyethyl)urea | 556 | 1.71 |
| 55c | 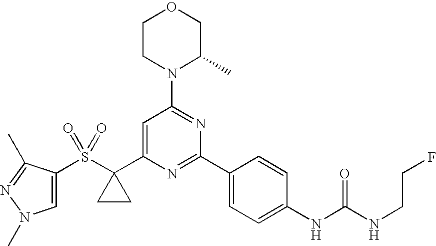 | 1-[4-[4-[1-(1,3-dimethylpyrazol-4-yl)sulfonylcyclopropyl]-6-[(3S)-3-methylmorpholin-4-yl]pyrimidin-2-yl]phenyl]-3-(2-fluoroethyl)urea | 558 | 2.00 |
| 55d | 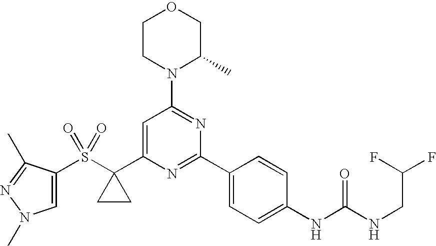 | 3-(2,2-difluoroethyl)-1-[4-[4-[1-(1,3-dimethylpyrazol-4-yl)sulfonylcyclopropyl]-6-[(3S)-3-methylmorpholin-4-yl]pyrimidin-2-yl]phenyl]urea | 576 | 2.13 |
| 55e | 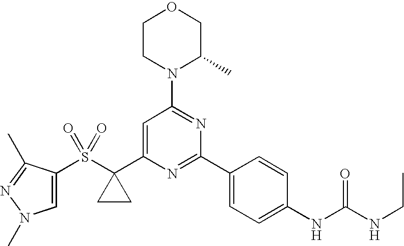 | 3-[4-[4-[1-(1,3-dimethylpyrazol-4-yl)sulfonylcyclopropyl]-6-[(3S)-3-methylmorpholin-4-yl]pyrimidin-2-yl]phenyl]-1-ethylurea | 540 | 2.00 |
| 55f | 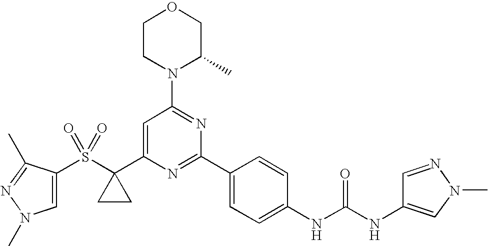 | 1-[4-[4-[1-(1,3-dimethylpyrazol-4-yl)sulfonylcyclopropyl]-6-[(3S)-3-methylmorpholin-4-yl]pyrimidin-2-yl]phenyl]-3-(1-methylpyrazol-4-yl)urea | 592 | 1.92 |

### EXAMPLE 55a

¹H NMR (399.902 MHz, DMSO-d₆) δ 0.42 (2H, m), 0.65 (2H, m), 1.20 (3H, d), 1.54 (2H, m), 1.74 (2H, m), 2.03 (3H, s), 2.56 (1H, m), 3.18 (1H, m), 3.48 (1H, m), 3.63 (1H, m), 3.76 (4H, m), 3.98 (1H, m), 4.14 (1H, m), 4.45 (1H, m), 6.42 (1H, m), 6.70 (1H, s), 7.46 (2H, m), 7.99 (2H, m), 8.19 (1H, s), 8.53 (1H, s)

### EXAMPLE 55b

¹H NMR (399.902 MHz, DMSO-d₆) δ 1.21 (3H, d), 1.54 (2H, m), 1.73 (2H, m), 2.03 (3H, s), 3.18 (3H, m), 3.47 (3H, m), 3.63 (1H, m), 3.76 (4H, m), 3.98 (1H, m), 4.14 (1H, m), 4.46 (1H, m), 4.74 (1H, t), 6.25 (1H, m), 6.70 (1H, s), 7.44 (2H, d), 7.98 (2H, d), 8.19 (1H, s), 8.79 (1H, s)

### EXAMPLE 55c

¹H NMR (399.902 MHz, DMSO-d₆) δ 1.20 (3H, d), 1.54 (2H, m), 1.74 (2H, m), 2.03 (3H, s), 3.18 (2H, m), 3.39 (1H, m), 3.46 (2H, m), 3.63 (1H, m), 3.76 (4H, m), 3.98 (1H, m), 4.14 (1H, m), 4.49 (2H, m), 6.43 (1H, m), 6.70 (1H, s), 7.46 (2H, d), 7.99 (2H, d), 8.18 (1H, s), 8.80 (1H, s)

### EXAMPLE 55d

¹H NMR (399.902 MHz, DMSO-d₆) δ 1.21 (3H, d), 1.55 (2H, m), 1.74 (2H, m), 2.02 (3H, s), 3.17 (1H, m), 3.54 (4H, m), 3.76 (4H, m), 3.97 (1H, m), 4.14 (1H, m), 4.47 (1H, m), 6.08 (1H, m), 6.52 (1H, m), 6.71 (1H, s), 7.46 (2H, m), 8.01 (2H, m), 8.19 (1H, s), 8.90 (1H, s)

### EXAMPLE 55e

¹H NMR (399.902 MHz, DMSO-d₆) δ 1.07 (3H, t), 1.20 (3H, d), 1.54 (2H, m), 1.74 (2H, m), 2.03 (3H, s), 3.15 (3H, m), 3.48 (1H, m), 3.63 (1H, m), 3.76 (4H, m), 3.98 (1H, m), 4.13 (1H, m), 4.46 (1H, m), 6.16 (1H, m), 6.70 (1H, s), 7.45 (2H, d), 7.98 (2H, d), 8.19 (1H, s), 8.65 (1H, s)

### EXAMPLE 55f

¹H NMR (399.902 MHz, DMSO-d₆) δ 1.21 (3H, d), 1.54 (2H, m), 1.74 (2H, m), 2.04 (3H, s), 3.18 (1H, m), 3.49 (1H, m), 3.63 (1H, m), 3.77 (7H, m), 3.98 (1H, m), 4.15 (1H, m), 4.47 (1H, m), 6.71 (1H, s), 7.39 (1H, s), 7.50 (2H, d), 7.77 (1H, s), 8.02 (2H, d), 8.19 (1H, s), 8.38 (1H, s), 8.82 (1H, s)

The preparation of phenyl N-[4-[4-[1-(1,3-dimethylpyrazol-4-yl)sulfonylcyclopropyl]-6-[(3S)-3-methylmorpholin-4-yl]pyrimidin-2-yl]phenyl]carbamate is described below:

### Phenyl N-[4-[4-[1-(1,3-dimethylpyrazol-4-yl)sulfonylcyclopropyl]-6-[(3S)-3-methylmorpholin-4-yl]pyrimidin-2-yl]phenyl]carbamate

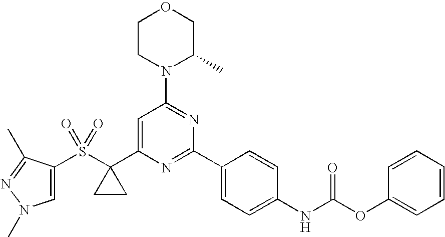

Phenyl chloroformate (0.201 mL, 1.60 mmol) was added dropwise to 4-[4-[1-(1,3-dimethylpyrazol-4-yl)sulfonylcyclopropyl]-6-[(3S)-3-methylmorpholin-4-yl]pyrimidin-2-yl]aniline (750 mg, 1.60 mmol) and sodium bicarbonate (148 mg, 1.76 mmol) in dioxane (30 mL). The resulting suspension was stirred at RT for 3 hours. The reaction mixture was filtered and the precipitate was redissolved in DCM (50 mL), and washed sequentially with water (50 mL) and saturated brine (50 mL). The organic layer was dried over MgSO₄, filtered and evaporated to afford the desired material as an orange gum which formed a foam solid when heated in desiccator (1.030 g).

NMR Spectrum: ¹H NMR (399.902 MHz, DMSO-d₆) δ 1.22 (3H, d), 1.55 (2H, m), 1.75 (2H, m), 2.04 (3H, s), 3.20 (1H, m), 3.49 (1H, m), 3.63 (1H, m), 3.76 (4H, m), 3.98 (1H, m), 4.16 (1H, m), 4.48 (1H, m), 6.75 (1H, s), 7.28 (3H, m), 7.45 (2H, m), 7.59 (2H, m), 8.07 (2H, m), 8.20 (1H, s), 10.43 (1H, s)

LCMS Spectrum: m/z (ESI+)(M+H)+=589; HPLC tR=2.63 min.

### 4-[4-[1-(1,3-Dimethylpyrazol-4-yl)sulfonylcyclopropyl]-6-[(3S)-3-methylmorpholin-4-yl]pyrimidin-2-yl]aniline

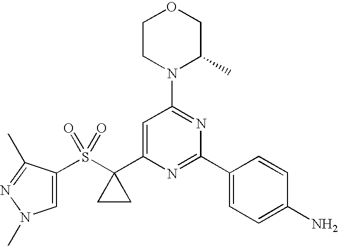

A solution of 2-chloro-4-[1-(1,3-dimethylpyrazol-4-yl)sulfonylcyclopropyl]-6-[(3S)-3-methylmorpholin-4-yl]pyrimidine (2.44 g, 5.92 mmol), 4-(4,4,5,5-tetramethyl-1,3,2-dioxaborolan-2-yl)aniline (1.428 g, 6.52 mmol) and sodium carbonate (2.51 g, 23.69 mmol) in DME (60 mL) and water (15 mL) was stirred under nitrogen for 5 minutes. Dichlorobis(triphenylphosphine)palladium(II) (0.416 g, 0.59 mmol) was added and the resulting solution was stirred at 80° C. for 5 hours. The reaction mixture was diluted with DCM (200 mL), and washed sequentially with water (200 mL) and saturated brine (200 mL). The organic layer was dried over MgSO₄, filtered and evaporated to afford crude product. The crude product was purified by flash silica chromatography, elution gradient 0 to 1% MeOH in DCM, to give crude product. The crude product was further purified by ion exchange chromatography using an SCX column, eluting with 2M ammonia in methanol, to give the desired material as a orange solid (1.25 g).

NMR Spectrum: ¹H NMR (399.902 MHz, DMSO-d₆) δ 1.19 (3H, d), 1.52 (2H, m), 1.72 (2H, m), 2.04 (3H, s), 3.15 (1H, m), 3.47 (1H, m), 3.61 (1H, m), 3.75 (4H, m), 3.97 (1H, m), 4.09 (1H, m), 4.42 (1H, m), 5.52 (2H, s), 6.57 (3H, m), 7.82 (2H, m), 8.17 (1H, s)

LCMS Spectrum: m/z (ESI+)(M+H)+=469; HPLC tR=1.87 min.

### 2-Chloro-4-[1-(1,3-dimethylpyrazol-4-yl)sulfonylcyclopropyl]-6-[(3S)-3-methylmorpholin-4-yl]pyrimidine

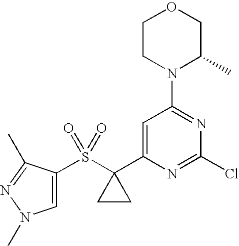

Sodium hydroxide (14.54 g, 363.47 mmol) in water (14.5 mL) was added to a stirred solution of 2-chloro-4-[(1,3-dimethylpyrazol-4-yl)sulfonylmethyl]-6-[(3S)-3-methylmorpholin-4-yl]pyrimidine (2.55 g, 6.61 mmol), 1,2-dibromoethane (1.708 mL, 19.83 mmol) and tetrabutylammonium bromide (0.213 g, 0.66 mmol) in toluene (100 mL). The resulting solution was stirred at 60° C. for 3 hours. The reaction mixture was diluted with DCM (200 mL), and washed sequentially with water (200 mL) and saturated brine (200 mL). The organic layer was dried over MgSO₄, filtered and evaporated to afford crude product. The crude product was purified by flash silica chromatography, elution gradient 0 to 30% ethyl acetate in DCM, to give the desired material as a white solid (2.44 g).

NMR Spectrum: ¹H NMR (399.902 MHz, DMSO-d₆) δ 1.18 (3H, d), 1.48 (2H, m), 1.70 (2H, m), 2.05 (3H, s), 3.17 (1H, m), 3.42 (1H, m), 3.57 (1H, m), 3.72 (1H, m), 3.80 (3H, s), 3.93 (2H, m), 4.30 (1H, m), 6.77 (1H, s), 8.20 (1H, s)

LCMS Spectrum: m/z (ESI+)(M+H)+=412; HPLC tR=1.69 min.

### 2-Chloro-4-[(1,3-dimethylpyrazol-4-yl)sulfonylmethyl]-6-[(3S)-3-methylmorpholin-4-yl]pyrimidine

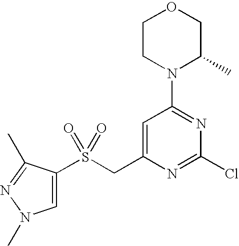

2-Chloro-4-(iodomethyl)-6-[(3S)-3-methylmorpholin-4-yl]pyrimidine (5 g, 14.14 mmol) was added to sodium 1,3-dimethyl-1H-pyrazole-4-sulfinate (4.79 g, 26.29 mmol) in DMF (80 mL). The resulting solution was stirred at RT for 18 hours. The reaction mixture was evaporated to dryness and redissolved in DCM (100 mL), and washed sequentially with water (100 mL) and saturated brine (100 mL). The organic layer was dried over MgSO₄, filtered and evaporated to afford crude product. The crude product was purified by flash silica chromatography, elution gradient 0 to 20% ethyl acetate in DCM, to give the desired material as a white solid (4.55 g).

NMR Spectrum: ¹H NMR (399.902 MHz, DMSO-d₆) δ 1.19 (3H, d), 2.13 (3H, s), 3.18 (1H, m), 3.44 (1H, m), 3.58 (1H, m), 3.73 (1H, m), 3.80 (3H, s), 3.94 (2H, m), 4.22 (1H, s), 4.46 (2H, s), 6.72 (1H, s), 8.14 (1H, s)

LCMS Spectrum: m/z (ESI+)(M+H)+=386; HPLC tR=1.63 min.

The preparation of 2-chloro-4-(iodomethyl)-6-[(3S)-3-methylmorpholin-4-yl]pyrimidine was described earlier.

### Sodium 1,3-dimethyl-1H-pyrazole-4-sulfinate

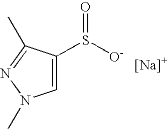

A solution of sodium sulfite (2.82 g, 22.35 mmol) in water (30 mL) was stirred for 10 minutes at RT. Sodium bicarbonate (3.75 g, 44.70 mmol) was added and the solution was stirred for 1 hour at 50° C. 1,3-Dimethyl-1H-pyrazole-4-sulfonyl chloride (4.35 g, 22.35 mmol) was added portionwise and the resulting solution was stirred at 50° C. for 18 hours. The suspension was allowed to stir at RT for 20 minutes. The suspension was filtered and the filtrate evaporated to afford the desired material as a white solid, which was air dried overnight under vacuum and used without further purification (4.79 g).

### EXAMPLE 56

### 3-Cyclopropyl-1-[4-[4-morpholin-4-yl-6-(1-Pyridin-2-ylsulfonylcyclopropyl)pyrimidin-2-yl]phenyl]urea

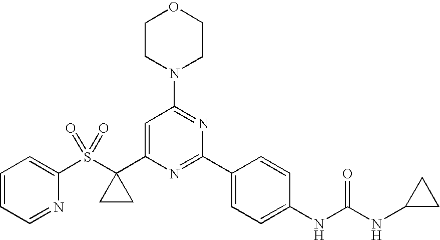

A solution of phenyl N-[4-[4-morpholin-4-yl-6-(1-pyridin-2-ylsulfonylcyclopropyl)pyrimidin-2-yl]phenyl]carbamate (0.100 g, 0.18 mmol), cyclopropylamine (0.90 mmol) and triethylamine (0.076 mL, 0.54 mmol) in NMP (2 mL) was heated at 50° C. for 16 hours. The crude product was purified by preparative HPLC, using decreasingly polar mixtures of water (containing 1% NH3) and MeCN as eluents, to give the desired material as a solid (75 mg).

NMR Spectrum: ¹H NMR (400.132 MHz, DMSO-d₆) δ 0.40-0.44 (2H, m), 0.63-0.67 (2H, m), 1.71-1.74 (2H, q), 1.98-2.01 (2H, q), 2.54-2.58 (1H, m), 3.63-3.65 (4H, m), 3.68-3.70 (4H, m), 6.40-6.41 (1H, d), 6.71 (1H, s), 7.35-7.37 (2H, d), 7.68-7.70 (2H, d), 7.73-7.76 (1H, m), 7.96-7.99 (1H, dt), 8.06-8.10 (1H, td), 8.49 (1H, s), 8.82-8.83 (1H, m).

LCMS Spectrum: m/z (ESI+)(M+H)+=521; HPLC tR=1.99 min.

The compounds below were prepared in an analogous fashion from phenyl N-[4-[4-morpholin-4-yl-6-(1-pyridin-2-ylsulfonylcyclopropyl)pyrimidin-2-yl]phenyl]carbamate using the appropriate amine.

| | | | | Retention |
|---|---|---|---|---|
| | | | LCMS | time |
| Example | Structure | NAME | MH+ | (min) |
| 56a | 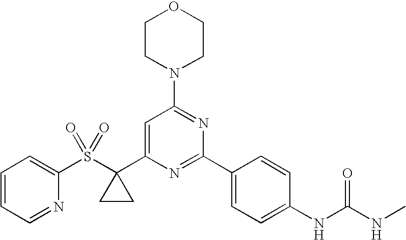 | 3-methyl-1-[4-[4-morpholin-4-yl-6-(1-pyridin-2-ylsulfonylcyclopropyl)pyrimidin-2-yl]phenyl]urea | 495 | 1.82 |
| 56b | 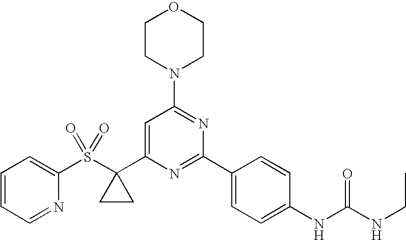 | 3-ethyl-1-[4-[4-morpholin-4-yl-6-(1-pyridin-2-ylsulfonylcyclopropyl)pyrimidin-2-yl]phenyl]urea | 509 | 1.96 |
| 56c | 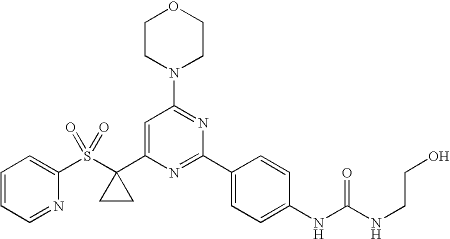 | 3-(2-hydroxyethyl)-1-[4-[4-morpholin-4-yl-6-(1-pyridin-2-ylsulfonylcyclopropyl)pyrimidin-2-yl]phenyl]urea | 525 | 1.65 |
| 56d | 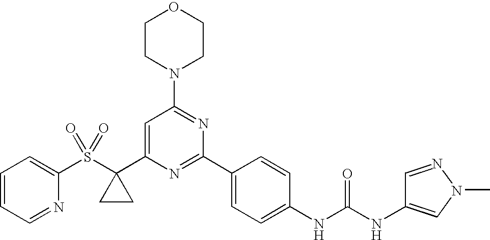 | 3-(1-methylpyrazol-4-yl)-1-[4-[4-morpholin-4-yl-6-(1-pyridin-2-ylsulfonylcyclopropyl)pyrimidin-2-yl]phenyl]urea | 561 | 1.88 |

### EXAMPLE 56a

¹H NMR (400.132 MHz, DMSO-d₆) δ 1.70-1.74 (2H, q), 1.98-2.01 (2H, q), 2.65-2.66 (3H, d), 3.63-3.65 (4H, m), 3.68-3.70 (4H, m), 6.02-6.06 (1H, q), 6.71 (1H, s), 7.34-7.37 (2H, d), 7.67-7.70 (2H, d), 7.73-7.76 (1H, m), 7.96-7.98 (1H, dt), 8.06-8.10 (1H, td), 8.69 (1H, s), 8.82-8.84 (1H, m).

### EXAMPLE 56b

¹H NMR (400.132 MHz, DMSO-d₆) δ 1.05-1.08 (3H, t), 1.70-1.73 (2H, q), 1.98-2.01 (2H, q), 3.09-3.16 (2H, m), 3.63-3.64 (4H, m), 3.68-3.69 (4H, m), 6.12-6.15 (1H, t), 6.71 (1H, s), 7.33-7.36 (2H, d), 7.67-7.70 (2H, d), 7.73-7.76 (1H, m), 7.96-7.99 (1H, dt), 8.06-8.10 (1H, td), 8.61 (1H, s), 8.82-8.83 (1H, m).

### EXAMPLE 56c

¹H NMR (400.132 MHz, DMSO-d₆) δ 1.70-1.74 (2H, q), 1.98-2.01 (2H, q), 3.15-3.19 (2H, m), 3.44-3.48 (2H, m), 3.63-3.65 (4H, m), 3.68-3.69 (4H, m), 4.71-4.74 (1H, t), 6.23-6.24 (1H, t), 6.71 (1H, s), 7.33-7.35 (2H, d), 7.68-7.70 (2H, d), 7.73-7.76 (1H, m), 7.96-7.99 (1H, dt), 8.06-8.10 (1H, td), 8.76 (1H, s), 8.82-8.84 (1H, m).

### EXAMPLE 56d

¹H NMR (400.132 MHz, DMSO-d₆) δ 1.71-1.74 (2H, q), 1.99-2.02 (2H, q), 3.64-3.66 (4H, m), 3.68-3.71 (4H, m), 3.79 (3H, s), 6.73 (1H, s), 7.38-7.41 (3H m), 7.71-7.76 (4H, m), 7.97-7.99 (1H, dt), 8.07-8.11 (1H, td), 8.36 (1H, s), 8.79 (1H, s), 8.83-8.84 (1H, m).

The preparation of phenyl N-[4-[4-morpholin-4-yl-6-(1-pyridin-2-ylsulfonylcyclopropyl)pyrimidin-2-yl]phenyl]carbamate is described below.

### Phenyl N-[4-[4-morpholin-4-yl-6-(1-pyridin-2-ylsulfonylcyclopropyl)pyrimidin-2-yl]phenyl]carbamate

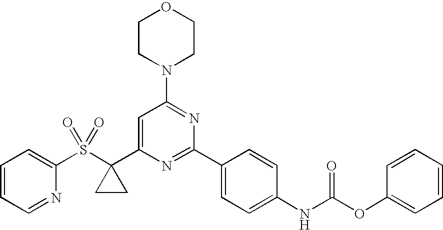

Phenyl chloroformate (0.363 mL, 2.89 mmol) was added to 4-[4-morpholin-4-yl-6-(1-pyridin-2-ylsulfonylcyclopropyl)pyrimidin-2-yl]aniline (1.265 g, 2.89 mmol) and sodium hydrogen carbonate (0.364 g, 4.34 mmol) in DCM (50 mL) at RT and the resulting suspension stirred for 2 hours. The mixture was washed with water, dried over MgSO₄, filtered and evaporated and the resultant gum dried in the vacuum oven at 50° C. overnight to give the desired material as a colorless gum (1.86 g).

NMR Spectrum: ¹H NMR (400.132 MHz, DMSO-d₆) δ 1.73-1.77 (2H, q), 1.99-2.03 (2H, q), 3.68-3.69(8H, m), 6.77 (1H, s), 7.24-7.32 (3H, m), 7.44-7.52 (4H, m), 7.74-7.77 (1H, m), 7.80-7.82 (2H, d), 7.98-8.01 (1H, dt), 8.07-8.11 (1H, td), 8.83-8.85 (1H, dq), 10.4 (1H, s).

LCMS Spectrum: m/z (ES+)(M+H)+=558; HPLC tR=2.75 min.

### 4-[4-morpholin-4-yl-6-(1-pyridin-2-ylsulfonylcyclopropyl)pyrimidin-2-yl]aniline

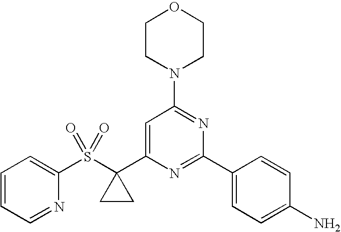

Bis(triphenylphosphine)palladium(II) chloride (0.131 g, 0.19 mmol) was added in one portion to 2-chloro-4-morpholin-4-yl-6-(1-pyridin-2-ylsulfonylcyclopropyl)pyrimidine (1.42 g, 3.73 mmol), 4-(4,4,5,5-tetramethyl-1,3,2-dioxaborolan-2-yl)aniline (0.817 g, 3.73 mmol) and 2M aquoeus sodium carbonate solution (9.32 mL, 18.64 mmol) in a DMF solution (18% DMF, 82% of a 7:3:2 mixture of DME:water:Ethanol) at 22° C. under nitrogen. The resulting solution was stirred at 80° C. for 3 hours. The solvent was removed, ethyl acetate added and the organics washed with water. Precipitate was observed in the aqueous layer and was isolated by filtration to give crude product. This material was dissolved in DCM and insoluble material removed by filtration and discarded. The filtrate was purified by ion exchange chromatography using an SCX column, eluting with 7M ammonia in methanol, to give material which was further purified by flash silica chromatography, elution gradient 0 to 60% ethyl acetate in DCM, to give the desired material as a white solid (1.265 g).

NMR Spectrum: ¹H NMR (400.132 MHz, DMSO-d₆) δ 1.68-1.71 (2H, q), 1.96-2.00 (2H, q), 3.59-3.61 (4H, m), 3.66-3.69 (4H, m), 5.48-5.50 (1H, d (NH2), 6.45-6.47 (2H, d), 6.61 (1H, s), 7.52-7.54 (2H, d), 7.72-7.75 (1H, m), 7.96-7.98 (1H, dt), 8.05-8.10 (1H, td), 8.81-8.83 (1H, dq).

LCMS Spectrum: m/z (ES+)(M+H)+=438; HPLC tR=1.93 min.

### 2-Chloro-4-morpholin-4-yl-6-(1-pyridin-2-ylsulfonylcyclopropyl)pyrimidine

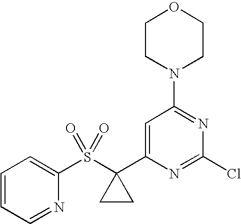

Sodium hydroxide (50% w/w solution) (12.71 g, 317.77 mmol) was added to 2-chloro-4-morpholin-4-yl-6-(pyridin-2-ylsulfonylmethyl)pyrimidine (2.050 g, 5.78 mmol), 1,2-dibromoethane (1.494 mL, 17.33 mmol) and tetrabutylammonium bromide (0.186 g, 0.58 mmol) in toluene (50 mL) at RT. The resulting suspension was stirred at 60° C. overnight. Water was added and the layers were separated. The organic layer was washed twice with water, dried over MgSO₄, filtered and evaporated. The crude product was purified by flash silica chromatography, elution gradient 0 to 40% ethyl acetate in DCM, to give the desired material as a white solid (1.42 g).

NMR Spectrum: ¹H NMR (400.132 MHz, DMSO-d₆) δ 1.63-1.66 (2H, q), 1.91-1.95 (2H, q), 3.55 (4H, bs), 3.63-3.65 (4H, t), 6.84 (1H, s), 7.73-7.76 (1H, m), 7.98-8.00 (1H, dt), 8.10-8.14 (1H, td), 8.77-8.79 (1H, dt).

LCMS Spectrum: m/z (ES+)(M+H)+=381; HPLC tR=1.71 min.

### 2-Chloro-4-morpholin-4-yl-6-(pyridin-2-ylsulfonylmethyl)pyrimidine

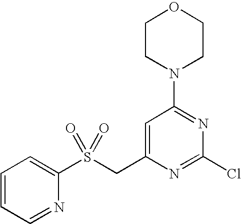

2-Chloro-4-morpholin-4-yl-6-(pyridin-2-ylsulfanylmethyl)pyrimidine (4.96 g, 15.36 mmol) was dissolved in dioxane (70 mL) and 2N sulfuric acid (0.362 mL) was added. The solution was heated to 55° C. Sodium tungstate dihydrate (0.101 g, 0.31 mmol) dissolved in water (3.54 mL) was added to the solution and allowed to stir for 10 minutes. Hydrogen peroxide (9.50 mL, 307.30 mmol) was then added dropwise over several minutes. The solution was heated at 55° C. for 4 hours. Water (300 mL) was added and the reaction was allowed to cool. The reaction mixture was extracted with DCM, the organic layer dried over MgSO₄, filtered and evaporated to afford desired product as a pale yellow solid (5.09 g).

NMR Spectrum: ¹H NMR (400.132 MHz, CDCl₃) δ 3.64 (3H, bs), 3.75-3.77(5H, t), 4.56 (2H, s), 6.60 (1H, s), 7.57-7.61 (1H, m), 7.97-7.97 (2H, m), 8.78-8.80 (1H, dt).

LCMS Spectrum: m/z (ES+)(M+H)+=355; HPLC tR=1.51 min.

### 2-Chloro-4-morpholin-4-yl-6-(pyridin-2-ylsulfanylmethyl)pyrimidine

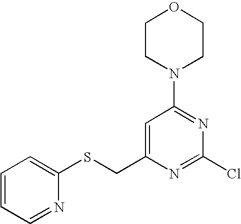

2-Chloro-4-(iodomethyl)-6-morpholin-4-ylpyrimidine (6.60 g, 19.44 mmol) was added to 2-mercaptopyridine (3.24 g, 29.16 mmol) and DIPEA (5.08 mL, 29.16 mmol) in acetonitrile (140 mL) at RT. The resulting solution was stirred at RT for 2 hours then evaporated to dryness, redissolved in DCM and washed sequentially with water and saturated brine. The organic layer was dried over MgSO₄, filtered and evaporated to afford crude product. The crude product was purified by flash silica chromatography, elution gradient 0 to 30% ethyl acetate in DCM, to give the desired material as a beige oil which solidified on standing (4.96 g).

NMR Spectrum: ¹H NMR (400.132 MHz, CDCl₃) δ 3.52 (4H, bs), 3.64-3.67 (4H, t), 4.27 (2H, s), 6.58 (1H, s), 6.93-6.96 (1H, q), 7.11-7.13 (1H, dd), 7.41-7.45 (1H, td), 8.34-8.36 (1H, dt).

LCMS Spectrum: m/z (ES+)(M+H)+=323; HPLC tR=1.98 min.

The preparation of 2-chloro-⁴-(iodomethyl)-6-morpholin-4-ylpyrimidine was described earlier.

### EXAMPLE 57

### 1-[4-[4-[1-(2-Fluoro-4-methylaminophenyl)sulfonylcyclopropyl]-6-[(3S)-3-methylmorpholin-4-yl]pyrimidin-2-yl]phenyl]-3-methylurea

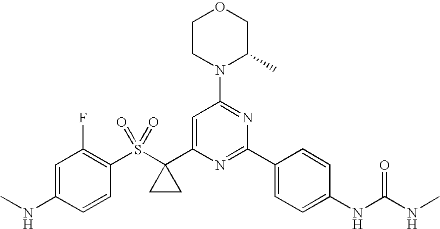

Phenyl N-[4-[4-[1-(2,4-difluorophenyl)sulfonylcyclopropyl]-6-[(3S)-3-methylmorpholin-4-yl]pyrimidin-2-yl]phenyl]carbamate (0.15 g, 0.25 mmol), triethylamine (0.103 mL, 0.74 mmol) and methylamine solution (2M in THF, 1.236 mL, 2.47 mmol) were added to dioxane (10 mL) and heated at 50° C. over the weekend. The reaction was evaporated to dryness and was purified by preparative HPLC, eluting with decreasingly polar mixtures of water (containing 1% ammonia) and acetonitrile, to give the desired material as a white solid (0.074 g).

NMR Spectrum: ¹H NMR (400.132 MHz, DMSO-d₆) δ 1.18 (3H, d), 1.51-1.48 (2H, m), 1.82-1.78 (2H, m), 2.66 (3H, d), 3.14 (1H, ddd), 3.45 (1H, ddd), 3.60 (1H, dd), 3.75 (1H, d), 3.96 (1H, dd), 4.06 (1H, d), 4.43 (1H, s), 6.04 (1H, q), 6.33-6.32 (1H, m), 6.38 (1H, dd), 6.43 (1H, ddd), 6.54 (1H, s), 7.47 (3H, m), 7.95 (2H, d), 8.72 (1H, s);

LCMS Spectrum: m/z (ES+)(M+H)+=555; HPLC tR=2.40 min.

The following compounds were prepared in an analogous fashion from phenyl N-[4-[4-[1-(2,4-difluorophenyl)sulfonylcyclopropyl]-6-[(3S)-3-methylmorpholin-4-yl]pyrimidin-2-yl]phenyl]carbamate and the appropriate amine.

| | | | | Reten- |
|---|---|---|---|---|
| | | | | tion |
| | | | LCMS | time |
| Example | Structure | NAME | MH+ | (min) |
| 57a | 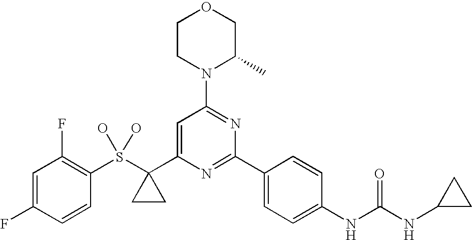 | 3-cyclopropyl-1-[4-[4-[1-(2,4-difluorophenyl)sulfonylcyclopro-pyl]-6-[(3S)-3-methylmorpholin-4-yl]pyrimidin-2-yl]phenyl]urea | 570 | 2.51 |
| 57b | 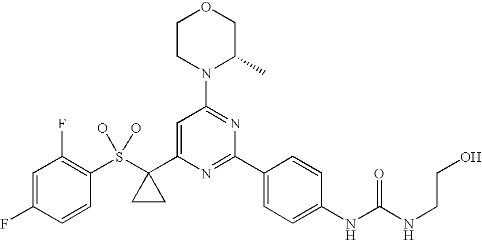 | 1-[4-[4-[1-(2,4-difluorophenyl)sulfonylcyclopro-pyl]-6-[(3S)-3-methylmorpholin-4-yl]pyrimidin-2-yl]phenyl]-3-(2-hydroxyethyl)urea | 573 | 1.96 |
| 57c | 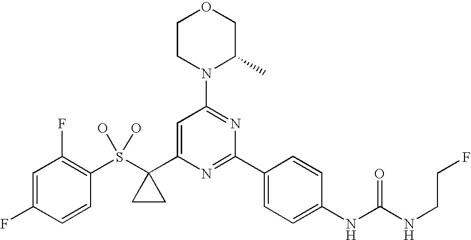 | 1-[4-[4-[1-(2,4-difluorophenyl)sulfonylcyclopro-pyl]-6-[(3S)-3-methylmorpholin-4-yl]pyrimidin-2-yl]phenyl]-3-(2-fluoroethyl)urea | 576 | 2.47 |
| 57d | 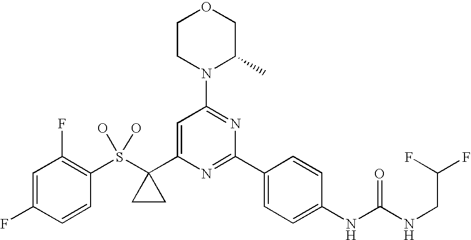 | 3-(2,2-difluoroethyl)-1-[4-[4-[1-(2,4-difluorophenyl)sulfonylcyclopro-pyl]-6-[(3S)-3-methylmorpholin-4-yl]pyrimidin-2-yl]phenyl]urea | 595 | 2.60 |
| 57e | 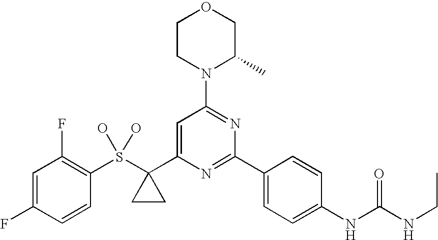 | 3-[4-[4-[1-(2,4-difluorophenyl)sulfonylcyclopro-pyl]-6-[(3S)-3-methylmorpholin-4-yl]pyrimidin-2-yl]phenyl]-1-ethylurea | 558 | 2.48 |
| 57f | 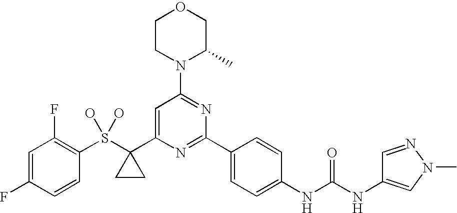 | 1-[4-[4-[1-(2,4-difluorophenyl)sulfonylcyclopro-pyl]-6-[(3S)-3-methylmorpholin-4-yl]pyrimidin-2-yl]phenyl]-3-(1-methylpyrazol-4-yl)urea | 610 | 2.35 |
| 57g | 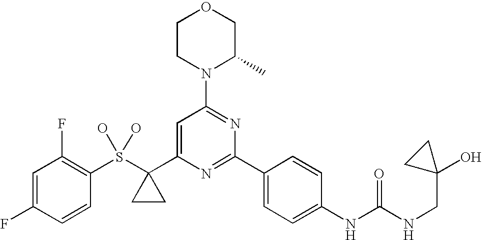 | 1-[4-[4-[1-(2,4-difluorophenyl)sulfonylcyclopro-pyl]-6-[(3S)-3-methylmorpholin-4-yl]pyrimidin-2-yl]phenyl]-3-[(1-hydroxycyclopropyl)methyl]urea | 600 | 2.31 |

### EXAMPLE 57a

¹H NMR (400.132 MHz, DMSO-d₆) δ 0.43-0.40 (2H, m), 0.67-0.62 (2H, m), 1.19 (3H, d), 1.67-1.64 (2H, m), 1.91-1.89 (2H, m), 2.57-2.54 (1H, m), 3.16 (1H, ddd), 3.46 (1H, ddd), 3.61 (1H, dd), 3.74 (1H, d), 3.95 (1H, dd), 4.16 (1H, d), 4.46 (1H, s), 6.39 (1H, s), 6.69 (1H, s), 7.20 (1H, ddd), 7.38 (2H, d), 7.65-7.59 (1H, m), 7.77-7.72 (3H, m), 8.51 (1H, s).

### EXAMPLE 57b

¹H NMR (400.132 MHz, DMSO-d₆) δ 1.19 (3H, d), 1.67-1.63 (2H, m), 1.91-1.87 (2H, m), 3.17 (3H, m), 3.48-3.42 (3H, m), 3.60 (1H, dd), 3.74 (1H, d), 3.95 (1H, d), 4.15 (1H, d), 4.46 (1H, s), 4.72 (1H, t), 6.22 (1H, t), 6.68 (1H, s), 7.20 (1H, ddd), 7.36 (2H, d), 7.65-7.60 (1H, m), 7.77-7.72 (3H, m), 8.78 (1H, s).

### EXAMPLE 57c

¹H NMR (400.132 MHz, DMSO-d₆) δ 1.19 (3H, d), 1.67-1.64 (2H, m), 1.91-1.89 (2H, m), 3.16 (1H, ddd), 3.38 (1H, q), 3.49-3.43 (2H, m), 3.61 (1H, dd), 3.74 (1H, d), 3.95 (1H, dd), 4.16 (1H, d), 4.41 (1H, t), 4.47 (1H, s), 4.53 (1H, t), 6.40 (1H, t), 6.69 (1H, s), 7.20 (1H, ddd), 7.38 (2H, d), 7.65-7.60 (1H, m), 7.77-7.71 (3H, m), 8.79 (1H, s).

### EXAMPLE 57d

¹H NMR (400.132 MHz, DMSO-d₆) δ 1.19 (3H, d), 1.67-1.64 (2H, m), 1.92-1.87 (2H, m), 3.16 (1H, ddd), 3.63-3.43 (4H, m), 3.74 (1H, d), 3.95 (1H, dd), 4.16 (1H, d), 4.47 (1H, s), 6.07 (1H, ddt), 6.49 (1H, t), 6.69 (1H, s), 7.20 (1H, t), 7.39 (1H, d), 7.65-7.60 (1H, m), 7.76-7.71 (4H, m), 8.90 (1H, s).

### EXAMPLE 57e

Spectrum not recorded.

### EXAMPLE 57f

¹H NMR (400.132 MHz, DMSO-d₆) δ 1.19 (3H, d), 1.67-1.64 (2H, m), 1.92-1.90 (2H, m), 3.16 (1H, ddd), 3.46 (1H, ddd), 3.61 (1H, dd), 3.75 (1H, d), 3.79 (3H, s), 3.96 (1H, d), 4.16 (1H, d), 4.48 (1H, s), 6.70 (1H, s), 7.20 (1H, ddd), 7.38 (1H, s), 7.42 (2H, d), 7.66-7.61 (1H, m), 7.78-7.71 (4H, m), 8.34 (1H, s), 8.82 (1H, s).

### EXAMPLE 57 g

¹H NMR (400.132 MHz, DMSO-d₆) δ 0.51-0.48 (2H, m), 0.60-0.57 (2H, m), 1.19 (3H, d), 1.67-1.62 (2H, m), 1.91-1.87 (2H, m), 3.22 (2H, d), 3.29-3.27 (1H, m), 3.46 (1H, ddd), 3.61 (1H, dd), 3.74 (1H, d), 3.95 (1H, dd), 4.17-4.14 (1H, m), 4.47 (1H, s), 6.29 (1H, t), 6.68 (1H, s), 7.20 (1H, t), 7.36 (2H, d), 7.63 (1H, t), 7.73 (3H, d), 8.78 (1H, s).

The preparation of phenyl N-[4-[4-[1-(2,4-difluorophenyl)sulfonylcyclopropyl]-6-[(3S)-3-methylmorpholin-4-yl]pyrimidin-2-yl]phenyl]carbamate is described below.

### Phenyl N-[4-[4-[1-(2,4-difluorophenyl)sulfonylcyclopropyl]-6-[(3S)-3-methylmorpholin-4-yl]pyrimidin-2-yl]phenyl]carbamate

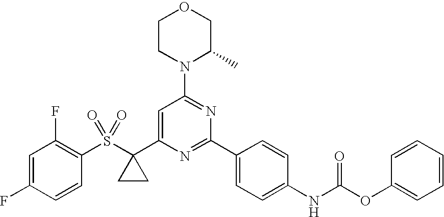

4-[4-[1-(2,4-Difluorophenyl)sulfonylcyclopropyl]-6-[(3S)-3-methylmorpholin-4-yl]pyrimidin-2-yl]aniline (2.1 g, 4.32 mmol) and sodium bicarbonate (0.725 g, 8.63 mmol) were added to dioxane (50 mL) and stirred for 10 minutes. Phenyl chloroformate (0.704 mL, 5.61 mmol) was added slowly and the reaction was stirred for 1 hour. The reaction mixture was quenched with 1.0 N citric acid (50 mL), extracted with ethyl acetate (3×75 mL), the organic layer was dried over MgSO4, filtered and evaporated to afford an orange solid. This was dissolved in DCM, the solvent was slowly removed until a solid was observed. Diethyl ether was then added to the solution with rapid stirring to afford the desired material as a white solid (1.65 g).

NMR Spectrum: ¹H NMR (400.132 MHz, DMSO-d₆) δ 1.20 (3H, d), 1.69-1.66 (2H, m), 1.92-1.89 (2H, m), 3.18 (1H, ddd), 3.46 (1H, ddd), 3.61 (1H, dd), 3.75 (1H, d), 3.96 (1H, dd), 4.36-4.10 (1H, m), 4.55-4.44 (1H, m), 6.75 (1H, s), 7.30-7.18 (4H, m), 7.47-7.43 (2H, m), 7.54 (2H, d), 7.64 (1H, t), 7.78-7.72 (1H, m), 7.85 (2H, d), 10.43 (1H, s).

LCMS Spectrum: m/z (ESI+) (M+H)+=607; HPLC tR=2.97 min

### 4-[4-[1-(2,4-Difluorophenyl)sulfonylcyclopropyl]-6-[(3S)-3-methylmorpholin-4-yl]pyrimidin-2-yl]aniline

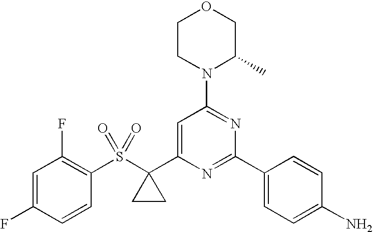

2-Chloro-4-[1-(2,4-difluorophenyl)sulfonylcyclopropyl]-6-[(3S)-3-methylmorpholin-4-yl]pyrimidine (3.61 g, 8.40 mmol), 4-(4,4,5,5-tetramethyl-1,3,2-dioxaborolan-2-yl)aniline (2.208 g, 10.08 mmol), sodium carbonate (4.45 g, 41.99 mmol) and 1,1′-bis(diphenylphosphino)ferrocenedichloropalladium(II) (0.608 g, 0.84 mmol) were added to DME (60.0 mL) and water (15 mL) and heated to 90° C. over a period of 2 hours under nitrogen. The solvent was evaporated and the residue was quenched water (100 mL), extracted with ethyl acetate (3×75 mL), the organic layer was dried over MgSO₄, filtered and evaporated to afford a black gum. The crude product was purified by flash silica chromatography, elution gradient 40 to 100% ethyl acetate in isohexane, to give the desired material as a yellow gum (2.2 g).

NMR Spectrum: ¹H NMR (400.132 MHz, CDCl₃) δ 1.29 (3H, d), 1.58-1.55 (1H, m), 1.66-1.62 (1H, m), 2.11-2.04 (2H, m), 3.26 (1H, ddd), 3.57 (1H, ddd), 3.72 (1H, dd), 3.80 (1H, d), 3.86 (2H, s), 4.02 (1H, dd), 4.11 (1H, m), 4.43-4.36 (1H, m), 6.60 (2H, d), 6.65 (1H, s), 6.86-6.82 (1H, m), 6.91 (1H, ddd), 7.75-7.69 (1H, m), 7.85 (2H, d).

LCMS Spectrum: m/z (ESI+) (M+H)+=487; HPLC tR=2.56 min.

### 2-Chloro-4-[1-(2,4-difluorophenyl)sulfonylcyclopropyl]-6-[(3S)-3-methylmorpholin-4-yl]pyrimidine

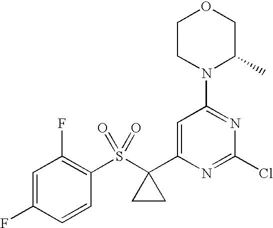

Sodium hydroxide (50% w/w solution) (32.7 g, 817.17 mmol) was added to 2-chloro-4-[(2,4-difluorophenyl)sulfonylmethyl]-6-[(3S)-3-methylmorpholin-4-yl]pyrimidine (6.0 g, 14.86 mmol), 1,2-dibromoethane (3.84 mL, 44.57 mmol) and tetrabutylammonium bromide (0.479 g, 1.49 mmol) in toluene (75 mL) at RT. The resulting suspension was stirred at 60° C. overnight. Water (100 mL) was added and the mixture was extracted with ethyl acetate (3×100 mL), dried over MgSO₄, filtered and evaporated. The crude product was purified by flash silica chromatography, elution gradient 0 to 40% ethyl acetate in DCM, to give a crude material which was dissolved in hot diethyl ether and then stirred for 2 hours to afford the desired material as a white solid (3.61 g).

NMR Spectrum: ¹H NMR (400.132 MHz, CDCl₃) δ 1.30 (3H, d), 1.56-1.52 (1H, m), 1.62-1.59 (1H, m), 2.10-2.00 (2H, m), 3.26 (1H, ddd), 3.53 (1H, ddd), 3.68 (1H, dd), 3.78 (1H, d), 4.00 (2H, dd), 4.26 (1H, s), 6.81 (1H, s), 6.99-6.90 (2H, m), 7.77-7.71 (1H, m).

LCMS Spectrum: m/z (ESI+) (M+H)+=430; HPLC tR=2.48 min

### 2-Chloro-4-[(2,4-difluorophenyl)sulfonylmethyl]-6-[(3S)-3-methylmorpholin-4-yl]pyrimidine

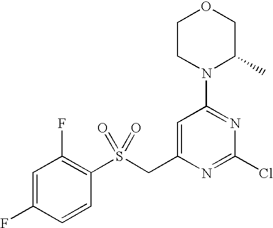

2,4-Difluorobenzenesulfinic acid, sodium salt (3.98 g, 19.80 mmol) and 2-chloro-4-(iodomethyl)-6-[(3S)-3-methylmorpholin-4-yl]pyrimidine (7 g, 19.80 mmol) were dissolved in DMF (50 mL) and stirred for 1 hour at RT. The solvent was evaporated to afford a brown gun, this was quenched with saturated aqueous ammonium chloride solution (50 mL), extracted with diethyl ether (3×75 mL), the organic layer was dried over MgSO₄, filtered and evaporated to afford a brown solid. The crude material was passed through a plug of silica, eluting with 80% ethyl acetate in isohexane, to give crude material which was triturated with diethyl ether to give the desired material as a white solid (7.04 g).

NMR Spectrum: ¹H NMR (399.902 MHz, CDCl₃) δ 1.32 (3H, d), 3.28 (1H, ddd), 3.54 (1H, ddd), 3.69 (1H, dd), 3.79 (1H, d), 4.03-3.99 (2H, m), 4.28 (1H, s), 4.43 (2H, s), 6.55 (1H, s), 7.03-6.98 (2H, m), 7.78-7.72 (1H, m).

LCMS Spectrum: m/z (ESI+) (M+H)+=404; HPLC tR=2.30 min;

The preparation of 2-chloro-4-(iodomethyl)-6-[(3S)-3-methylmorpholin-4-yl]pyrimidine was described earlier.

### 2,4-Difluorobenzenesulfinic acid, sodium salt

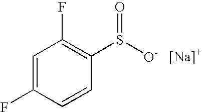

A solution of sodium sulfite (29.6 g, 235.18 mmol) in water (200 mL) was stirred at RT for 10 minutes. Sodium bicarbonate (39.5 g, 470.36 mmol) was added to the stirred solution. The resulting solution was stirred at 50° C. for 10 minutes. 2,4-Difluorobenzene-1-sulfonyl chloride (50 g, 235.18 mmol) was added portion wise to the solution and was stirred at 50° C. for 2 hours. The reaction mixture was evaporated to dryness and re-dissolved in methanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (41.6 g).

NMR Spectrum: ¹H NMR (399.902 MHz, DMSO-d₆) δ 7.10-7.04 (2H, m), 7.74-7.68 (1H, m);

### EXAMPLE 58

### 1-[4-[4-[1-(2-Fluorophenyl)sulfonylcyclopropyl]-6-[(3S)-3-methylmorpholin-4-yl]pyrimidin-2-yl]phenyl]-3-methylurea

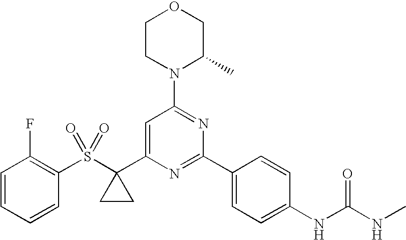

Phenyl N-[4-[4-[1-(2-fluorophenyl)sulfonylcyclopropyl]-6-[(3S)-3-methylmorpholin-4-yl]pyrimidin-2-yl]phenyl]carbamate (0.2 g, 0.34 mmol) and methylamine solution (2M in THF, 1.699 mL, 3.40 mmol) were added to dioxane (10 mL) and stirred overnight. The reaction was evaporated to dryness and was purified by preparative HPLC, eluting with decreasingly polar mixtures of water (containing 1% ammonia) and acetonitrile, to give the desired material as a white solid (0.119 g).

NMR Spectrum: ¹H NMR (400.132 MHz, DMSO-d₆) δ 1.17 (3H, d), 1.71-1.65 (2H, m), 1.94-1.89 (2H, m), 2.66 (3H, d), 3.14 (1H, ddd), 3.45 (1H, ddd), 3.60 (1H, dd), 3.74 (1H, d), 3.95 (1H, dd), 4.12 (1H, d), 4.42 (1H, s), 6.04 (1H, q), 6.66 (1H, s), 7.37-7.31 (3H, m), 7.52-7.48 (1H, m), 7.78-7.70 (4H, m), 8.69 (1H, s);

m/z (ESI+) (M+H)+=526; HPLC tR=2.34 min;

The following compounds were prepared in an analogous fashion from phenyl N-[4-[4-[1-(2-fluorophenyl)sulfonylcyclopropyl]-6-[(3S)-3-methylmorpholin-4-yl]pyrimidin-2-yl]phenyl]carbamate and the appropriate amine.

| | | | | Reten- |
|---|---|---|---|---|
| | | | | tion |
| | | | LCMS | time |
| Example | Structure | NAME | MH+ | (min) |
| 58a | 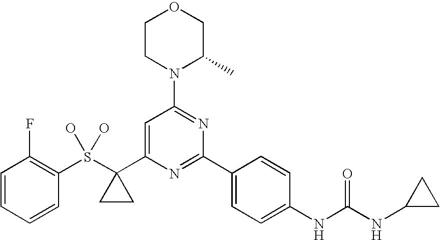 | 3-cyclopropyl-1-[4-[4-[1-(2-fluorophenyl)sulfonylcyclopropyl]-6-[(3S)-3-methylmorpholin-4-yl]pyrimidin-2-yl]phenyl]urea | 552 | 2.45 |
| 58b | 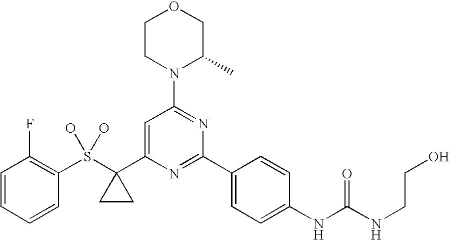 | 1-[4-[4-[1-(2-fluorophenyl)sulfonylcyclopropyl]-6-[(3S)-3-methylmorpholin-4-yl]pyrimidin-2-yl]phenyl]-3-(2-hydroxyethyl)urea | 556 | 2.09 |
| 58c | 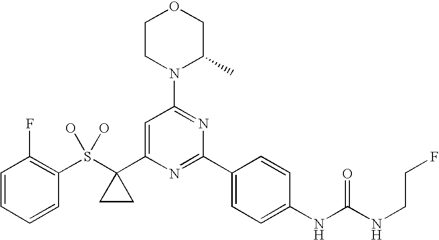 | 3-(2-fluoroethyl)-1-[4-[4-[1-(2-fluorophenyl)sulfonylcyclopropyl]-6-[(3S)-3-methylmorpholin-4-yl]pyrimidin-2-yl]phenyl]urea | 558 | 2.41 |
| 58d | 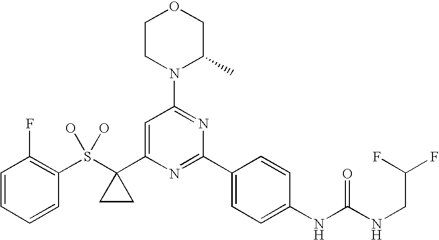 | 3-(2,2-difluoroethyl)-1-[4-[4-[1-(2-fluorophenyl)sulfonylcyclopropyl]-6-[(3S)-3-methylmorpholin-4-yl]pyrimidin-2-yl]phenyl]urea | 576 | 2.53 |
| 58e | 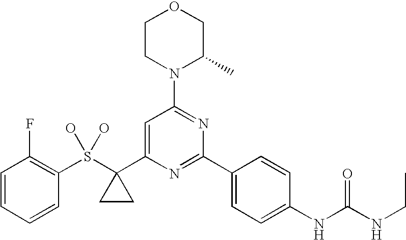 | 1-ethyl-3-[4-[4-[1-(2-fluorophenyl)sulfonylcyclopropyl]-6-[(3S)-3-methylmorpholin-4-yl]pyrimidin-2-yl]phenyl]urea | 540 | 2.41 |
| 58f | 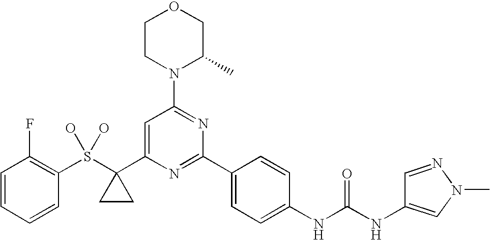 | 1-[4-[4-[1-(2-fluorophenyl)sulfonylcyclopropyl]-6-[(3S)-3-methylmorpholin-4-yl]pyrimidin-2-yl]phenyl]-3-(1-methylpyrazol-4-yl)urea | 591 | 2.31 |
| 58g | 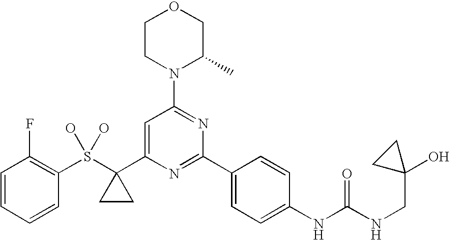 | 1-[4-[4-[1-(2-fluorophenyl)sulfonylcyclopropyl]-6-[(3S)-3-methylmorpholin-4-yl]pyrimidin-2-yl]phenyl]-3-[(1-hydroxycyclopropyl)methyl]urea | 582 | 2.26 |

### EXAMPLE 58a

¹H NMR (400.132 MHz, DMSO-d₆) δ 0.43-0.40 (2H, m), 0.67-0.62 (2H, m), 1.17 (3H, d), 1.71-1.65 (2H, m), 1.95-1.87 (2H, m), 2.58-2.51 (1H, m), 3.14 (1H, ddd), 3.45 (1H, ddd), 3.60 (1H, dd), 3.74 (1H, d), 3.95 (1H, dd), 4.12 (1H, d), 4.43 (1H, s), 6.40 (1H, d), 6.66 (1H, s), 7.37-7.31 (3H, m), 7.52-7.48 (1H, m), 7.79-7.70 (4H, m), 8.48 (1H, s);

### EXAMPLE 58b

¹H NMR (400.132 MHz, DMSO-d₆) δ 1.17 (3H, d), 1.71-1.65 (2H, m), 1.93-1.89 (2H, m), 3.19-3.10 (3H, m), 3.48-3.42 (3H, m), 3.60 (1H, dd), 3.74 (1H, d), 3.95 (1H, dd), 4.12 (1H, d), 4.43 (1H, s), 4.72 (1H, t), 6.23 (1H, t), 6.66 (1H, s), 7.35-7.31 (3H, m), 7.52-7.48 (1H, m), 7.78-7.70 (4H, m), 8.75 (1H, s);

### EXAMPLE 58c

¹H NMR (400.132 MHz, DMSO-d₆) δ 1.17 (3H, d), 1.71-1.65 (2H, m), 1.94-1.88 (2H, m), 3.14 (1H, ddd), 3.48-3.36 (3H, m), 3.60 (1H, dd), 3.74 (1H, d), 3.95 (1H, dd), 4.12 (1H, d), 4.46-4.40 (2H, m), 4.53 (1H, t), 6.41 (1H, t), 6.66 (1H, s), 7.37-7.31 (3H, m), 7.52-7.48 (1H, m), 7.78-7.69 (4H, m), 8.76 (1H, s);

### EXAMPLE 58d

¹H NMR (400.132 MHz, DMSO-d₆) δ 1.17 (3H, d), 1.71-1.67 (2H, m), 1.94-1.89 (2H, m), 3.14 (1H, ddd), 3.55-3.42 (3H, m), 3.60 (1H, dd), 3.74 (1H, d), 3.95 (1H, dd), 4.13 (1H, d), 4.42 (1H, s), 6.07 (1H, ddt), 6.51 (1H, t), 6.67 (1H, s), 7.38-7.31 (3H, m), 7.52-7.48 (1H, m), 7.78-7.70 (4H, m), 8.87 (1H, s);

### EXAMPLE 58e

¹H NMR (400.132 MHz, DMSO-d₆) δ 1.06 (3H, t), 1.17 (3H, d), 1.71-1.65 (2H, m), 1.94-1.88 (2H, m), 3.17-3.09 (3H, m), 3.45 (1H, ddd), 3.60 (1H, dd), 3.74 (1H, d), 3.95 (1H, ddd), 4.12 (1H, d), 4.42 (1H, s), 6.13 (1H, t), 6.66 (1H, s), 7.36-7.31 (3H, m), 7.52-7.48 (1H, m), 7.78-7.71 (4H, m), 8.60 (1H, s);

### EXAMPLE 58f

¹H NMR (400.132 MHz, DMSO-d₆) δ 1.18 (3H, d), 1.72-1.65 (2H, m), 1.94-1.89 (2H, m), 3.15 (1H, ddd), 3.46 (1H, ddd), 3.61 (1H, dd), 3.74 (1H, d), 3.79 (3H, s), 3.95 (1H, dd), 4.13 (1H, d), 4.43 (1H, s), 6.67 (1H, s), 7.34 (1H, t), 7.41-7.38 (3H, m), 7.53-7.49 (1H, m), 7.79-7.70 (5H, m), 8.35 (1H, s), 8.78 (1H, s).

### EXAMPLE 58g

¹H NMR (400.132 MHz, DMSO-d₆) δ 0.51-0.48 (2H, m), 0.59-0.57 (2H, m), 1.17 (3H, d), 1.71-1.65 (2H, m), 1.94-1.90 (2H, m), 3.14 (1H, ddd), 3.22 (2H, d), 3.45 (1H, ddd), 3.60 (1H, dd), 3.74 (1H, d), 3.95 (1H, dd), 4.12 (1H, d), 4.42 (1H, s), 5.36 (1H, s), 6.29 (1H, t), 6.66 (1H, s), 7.35-7.31 (3H, m), 7.52-7.48 (1H, m), 7.78-7.70 (4H, m), 8.76 (1H, s).

The preparation of phenyl N-[4-[4-[1-(2-fluorophenyl)sulfonylcyclopropyl]-6-[(3S)-3-methylmorpholin-4-yl]pyrimidin-2-yl]phenyl]carbamate is described below.

### Phenyl N-[4-[4-[1-(2-fluorophenyl)sulfonylcyclopropyl]-6-[(3S)-3-methylmorpholin-4-yl]pyrimidin-2-yl]phenyl]carbamate

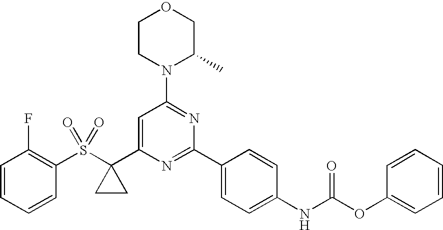

4-[4-[1-(2-Fluorophenyl)sulfonylcyclopropyl]-6-[(3S)-3-methylmorpholin-4-yl]pyrimidin-2-yl]aniline (used as the hydrochloride salt) (3.2 g, 6.83 mmol) and sodium bicarbonate (11.47 g, 136.59 mmol) were added to DCM (75 mL) and stirred for 10 minutes. Phenyl chloroformate (1 114 mL, 8.88 mmol) was added slowly and the reaction was stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. The crude product was purified by flash silica chromatography, elution gradient 30 to 50% ethyl acetate in isohexane, to give the desired material as a yellow foam (3.5 g).

NMR Spectrum: ¹H NMR (400.132 MHz, DMSO-d₆) δ 1.18 (3H, d), 1.72-1.66 (2H, m), 1.97-1.88 (2H, m), 3.15 (1H, ddd), 3.45 (1H, ddd), 3.60 (1H, dd), 3.74 (1H, d), 3.95 (1H, dd), 4.14 (1H, d), 4.44 (1H, s), 6.70 (1H, s), 7.35-7.23 (4H, m), 7.53-7.42 (5H, m), 7.77-7.71 (2H, m), 7.83 (2H, d), 10.37 (1H, s).

LCMS Spectrum: m/z (ESI+) (M+H)+=589; HPLC tR=3.04 min;

### 4-[4-[1-(2-Fluorophenyl)sulfonylcyclopropyl]-6-[(3S)-3-methylmorpholin-4-yl]pyrimidin-2-yl]aniline

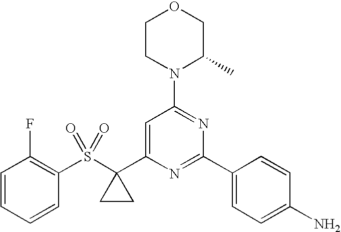

tert-Butyl N-[4-[4-[1-(2-fluorophenyl)sulfonylcyclopropyl]-6-[(3S)-3-methylmorpholin-4-yl]pyrimidin-2-yl]phenyl]carbamate (3.6 g, 6.33 mmol) was added to 6N hydrogen chloride in propan-2-ol (60 mL) and stirred at RT for 5 hours. The solvent was removed to 80% of the initial volume then diethyl ether rapidly added to afford the desired material (as a hydrochloride salt) as a yellow solid (3.20 g). This was used in the next step without any further purification.

### tert-Butyl N-[4-[4-[1-(2-fluorophenyl)sulfonylcyclopropyl]-6-[(3S)-3-methylmorpholin-4-yl]pyrimidin-2-yl]phenyl]carbamate

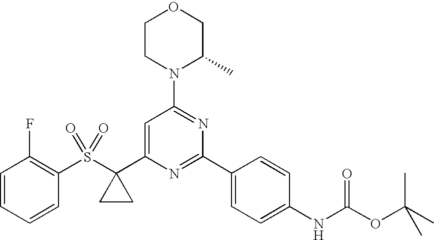

tert-Butyl N-[4-[4-[(2-fluorophenyl)sulfonylmethyl]-6-[(3S)-3-methylmorpholin-4-yl]pyrimidin-2-yl]phenyl]carbamate (5.75 g, 10.60 mmol) was added to sodium hydride (1.526 g, 31.79 mmol) and 1,2-dibromoethane (1.826 mL, 21.19 mmol) in DMF (30 mL) at RT. The resulting suspension was stirred at 45° C. for 1 hour. Additional sodium hydride (1.526 g, 31.79 mmol) and 1,2-dibromoethane (1.826 mL, 21.19 mmol) were added and the reaction was stirred at 45° C. overnight. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to give the desired material as a yellow foam (3.60 g).

NMR Spectrum: ¹H NMR (400.132 MHz, DMSO-d₆) δ 1.17 (3H, d), 1.49 (9H, s), 1.71-1.65 (2H, m), 1.94-1.90 (2H, m), 3.14 (1H, ddd), 3.45 (1H, ddd), 3.60 (1H, dd), 3.74 (1H, d), 3.95 (1H, ddd), 4.13 (1H, d), 4.42 (1H, s), 6.68 (1H, s), 7.33 (1H, t), 7.42 (2H, d), 7.53-7.48 (1H, m), 7.78-7.70 (4H, m), 9.48 (1H, s).

LCMS Spectrum: m/z (ESI+) (M+H)+=569; HPLC tR=3.13 min;

### tert-Butyl N-[4-[4-[(2-fluorophenyl)sulfonylmethyl]-6-[(3S)-3-methylmorpholin-4-yl]pyrimidin-2-yl]phenyl]carbamate

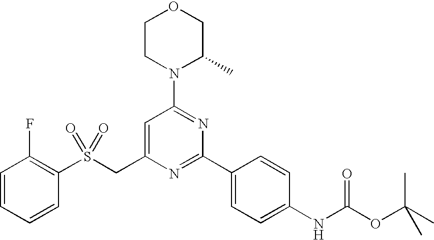

[6-[(3S)-3-Methylmorpholin-4-yl]-2-[4-[(2-methylpropan-2-yl)oxycarbonylamino]phenyl]pyrimidin-4-yl]methyl methanesulfonate (4.74 g, 9.90 mmol) and lithium iodide (3.98 g, 29.71 mmol) were added to dioxane (70 mL) and heated at 97° C. for 30 minutes. To this was added sodium 2-fluorobenzenesulfinate (2.71 g, 14.86 mmol) and DMF (5 mL), the reaction was stirred at 97° C. overnight. The reaction mixture was quenched with water (50 mL), extracted with ethyl acetate (3×50 mL), the organic layer was dried over MgSO₄, filtered and evaporated to afford an orange gum. Diethyl ether (100 mL) was added with vigorous stirring to afford the desired material as a white solid (4.0 g).

NMR Spectrum: ¹H NMR (400.132 MHz, DMSO-d₆) δ 1.20 (3H, d), 1.49 (9H, s), 3.17 (1H, ddd), 3.48 (1H, ddd), 3.63 (1H, dd), 3.76 (1H, d), 3.97 (1H, dd), 4.12 (1H, d), 4.39 (1H, s), 4.76 (2H, s), 6.75 (1H, s), 7.34 (1H, t), 7.41 (2H, d), 7.65-7.57 (2H, m), 7.71 (2H, d), 7.83-7.77 (1H, m), 9.48 (1H, s).

LCMS Spectrum: m/z (ESI+) (M+H)+=543; HPLC tR=2.92 min;

### Sodium 2-fluorobenzenesulfinate

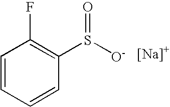

A solution of sodium sulfite (32.4 g, 256.92 mmol) in water (200 mL) was stirred at RT for 10 minutes. Sodium bicarbonate (43.2 g, 513.85 mmol) was added to the stirred solution. The resulting solution was stirred at 50° C. for 10 minutes. 2-Fluorobenzene-1-sulfonyl chloride (50 g, 256.92 mmol) was added portionwise to the solution and was stirred at 50° C. for 2 hours. The reaction mixture was evaporated to dryness and redissolved in methanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (41.0 g).

NMR Spectrum: ¹H NMR (400.132 MHz, DMSO-d₆) δ 6.99 (1H, t), 7.16 (1H, t), 7.28-7.22 (1H, m), 7.61 (1H, t).

### [6-[(3S)-3-Methylmorpholin-4-yl]-2-[4-[(2-methylpropan-2-yl)oxycarbonylamino]phenyl]pyrimidin-4-yl]methyl methanesulfonate

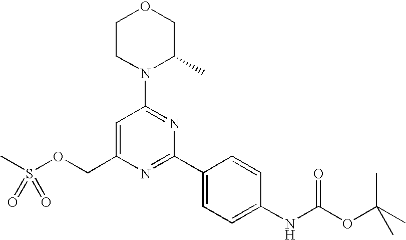

tert-Butyl N-[4-[4-(hydroxymethyl)-6-[(3S)-3-methylmorpholin-4-yl]pyrimidin-2-yl]phenyl]carbamate (23 g, 57.43 mmol) and DIPEA (12.04 mL, 68.92 mmol) were added to DCM (80 mL), to this was slowly added methanesulphonyl chloride (4.48 mL, 57.43 mmol) and the reaction was stirred for 30 minutes. The reaction mixture was quenched with a saturated aqueous solution of ammonium chloride (100 mL), extracted with DCM (2×100 mL), the organic layer was dried over MgSO₄, filtered and evaporated to afford the desired material as a brown gum (27.0 g). This was used without any further purification.

NMR Spectrum: ¹H NMR (400.132 MHz, DMSO-d₆) δ 1.24 (3H, d), 1.50 (9H, s), 3.22 (1H, ddd), 3.34 (3H, s), 3.50 (1H, ddd), 3.65 (1H, dd), 3.77 (1H, d), 3.98 (1H, dd), 4.19 (1H, d), 4.53 (1H, s), 5.19 (2H, s), 6.71 (1H, s), 7.56 (2H, d), 8.23 (2H, d), 9.55 (1H, s).

LCMS Spectrum: m/z (ESI+) (M+H)+=479; HPLC tR=2.74 min;

### tert-Butyl N-[4-[4-(hydroxymethyl)-6-[(3S)-3-methylmorpholin-4-yl]pyrimidin-2-yl]phenyl]carbamate

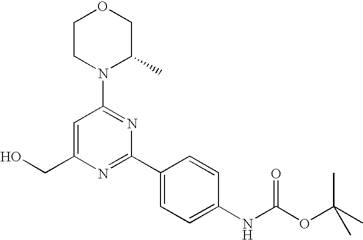

[2-Chloro-6-[(3S)-3-methylmorpholin-4-yl]pyrimidin-4-yl]methanol (18.00 g, 73.86 mmol), tert-butyl 4-(4,4,5,5-tetramethyl-1,3,2-dioxaborolan-2-yl)phenylcarbamate (23.58 g, 73.86 mmol), sodium carbonate (39.1 g, 369.32 mmol) and 1,1′-bis(diphenylphosphino)ferrocenedichloropalladium(II) (5.35 g, 7.39 mmol) were added to DME (300 mL) and water (75 mL) and heated to 90° C. overnight under nitrogen. The solvent was evaporated and the residue was quenched with water (100 mL), extracted with ethyl acetate (3×100 mL), the aqueous layer was dried over MgSO₄, filtered and evaporated to afford black gum. The residue was filtered through a plug of silica eluting with ethyl acetate to give a pale orange gum. This was triturated with diethyl ether to give the desired material as a white solid (24.4 g).

NMR Spectrum: ¹H NMR (400.132 MHz, DMSO-d₆) δ 1.23 (3H, d), 1.49 (9H, s), 3.19 (1H, ddd), 3.49 (1H, ddd), 3.64 (1H, dd), 3.76 (1H, d), 3.97 (1H, dd), 4.17 (1H, d), 4.50-4.46 (3H, m), 5.39 (1H, s), 6.67 (1H, s), 7.54 (2H, d), 8.22 (2H, d), 9.50 (1H, s).

LCMS Spectrum: m/z (ESI+) (M+H)+=401; HPLC tR=2.30 min;

The preparation of [2-chloro-6-[(3S)-3-methylmorpholin-4-yl]pyrimidin-4-yl]methanol was described earlier.

### EXAMPLE 59

### 1-[4-[4-[1-[(3,5-Dimethyl-1,2-oxazol-4-yl)sulfonyl]cyclopropyl]-6-methylmorpholin-4-yl]pyrimidin-2-yl]phenyl]-3-methylurea

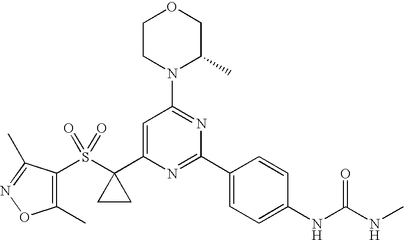

Phenyl N-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]carbamate (0.15 g, 0.25 mmol) and methylamine (2M in THF, 1.272 mL, 2.54 mmol) were added to dioxane (10 mL) and stirred overnight at 50° C. The reaction was evaporated to dryness and was purified by preparative HPLC, eluting with decreasingly polar mixtures of water (containing 1% ammonia) and acetonitrile, to give the desired material as a white solid (0.098 g).

NMR Spectrum: ¹H NMR (400.132 MHz, DMSO-d₆) δ 1.21 (3H, d), 1.59-1.52 (2H, m), 1.80-1.77 (2H, m), 2.11 (3H, s), 2.33 (3H, s), 2.66 (3H, d), 3.18 (1H, ddd), 3.47 (1H, ddd), 3.62 (1H, dd), 3.76 (1H, d), 3.97 (1H, dd), 4.17 (1H, d), 4.52 (1H, s), 6.07 (1H, q), 6.75 (1H, s), 7.45 (2H, d), 7.96 (2H, d), 8.72 (1H, s).

LCMS Spectrum: m/z (ESI+) (M+H)+=527; HPLC tR=2.18 min

The following samples were prepared in an analogous fashion from phenyl N-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]carbamate and the appropriate amine.

| | | | | Reten- |
|---|---|---|---|---|
| | | | | tion |
| | | | LCMS | time |
| Example | Structure | NAME | MH+ | (min) |
| 59a | 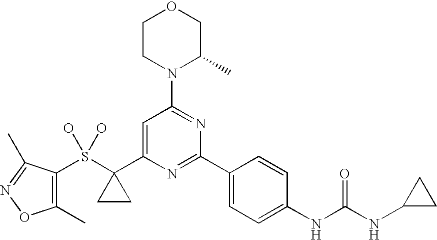 | 3-cyclopropyl-1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]urea | 553 | 2.31 |
| 59b | 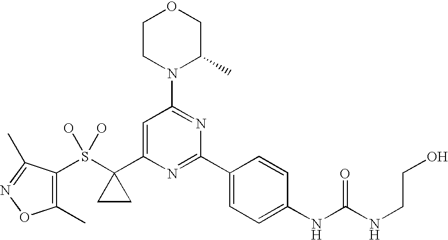 | 1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-(2-hydroxyethyl)urea | 557 | 2.00 |
| 59c | 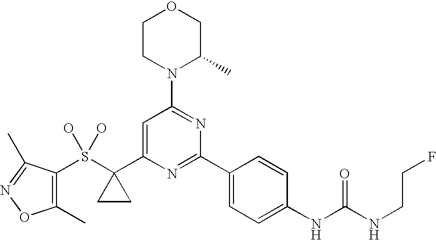 | 1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-(2-fluoroethyl)urea | 559 | 2.33 |
| 59d | 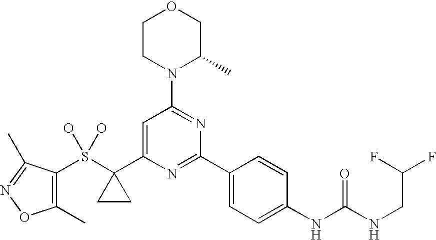 | 3-(2,2-difluoroethyl)-1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]urea | 577 | 2.44 |
| 59e | 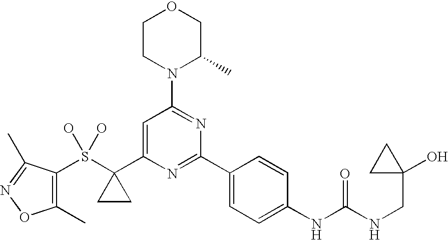 | 1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-[(1-hydroxycyclopropyl)methyl]urea | 583 | 2.16 |
| 59f | 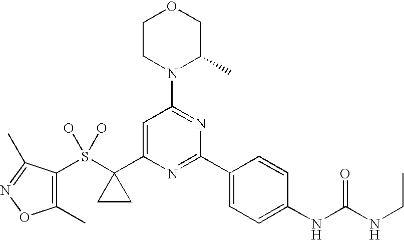 | 3-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-1-ethylurea | 541 | 2.34 |
| 59g | 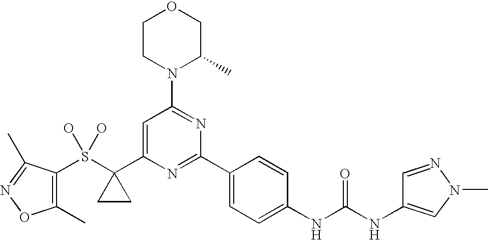 | 1-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]-3-(1-methylpyrazol-4-yl)urea | 593 | 2.21 |

### EXAMPLE 59a

¹H NMR (400.132 MHz, DMSO-d₆) δ 0.44-0.40 (2H, m), 0.67-0.63 (2H, m), 1.21 (3H, d), 1.59-1.52 (2H, m), 1.80-1.77 (2H, m), 2.11 (3H, s), 2.32 (3H, s), 2.59-2.54 (1H, m), 3.18 (1H, ddd), 3.47 (1H, ddd), 3.62 (1H, dd), 3.76 (1H, d), 3.97 (1H, dd), 4.17 (1H, d), 4.52 (1H, s), 6.44 (1H, d), 6.76 (1H, s), 7.45 (2H, d), 7.96 (2H, d), 8.52 (1H, s).

### EXAMPLE 59b

¹H NMR (400.132 MHz, DMSO-d₆) δ 1.20 (3H, d), 1.59-1.52 (2H, m), 1.80-1.75 (2H, m), 2.11 (3H, s), 2.32 (3H, s), 3.22-3.14 (3H, m), 3.51-3.45 (3H, m), 3.62 (1H, dd), 3.76 (1H, d), 3.97 (1H, dd), 4.17 (1H, d), 4.52 (1H, s), 4.73 (1H, t), 6.26 (1H, t), 6.75 (1H, s), 7.43 (2H, d), 7.96 (2H, d), 8.79 (1H, s).

### EXAMPLE 59c

¹H NMR (400.132 MHz, DMSO-d₆) δ 1.21 (3H, d), 1.59-1.53 (2H, m) 1.80-1.75 (2H, m), 2.11 (3H, s), 2.33 (3H, s), 3.22-3.14 (1H, m), 3.39 (1H, q), 3.51-3.44 (1H, m), 3.62 (1H, dd), 3.76 (1H, d), 3.97 (1H, dd), 4.17 (1H, d), 4.42 (1H, t), 4.55-4.49 (2H, m), 5.75 (1H, s), 6.44 (1H, t), 6.76 (1H, s), 7.45 (2H, d), 7.97 (2H, d), 8.79 (1H, s);

### EXAMPLE 59d

¹H NMR (400.132 MHz, DMSO-d₆) δ 1.21 (3H, d), 1.60-1.52 (2H, m), 1.81-1.75 (2H, m), 2.11 (3H, s), 2.33 (3H, s), 3.18 (1H, ddd), 3.64-3.44 (4H, m), 3.76 (1H, d), 3.97 (1H, dd), 4.18 (1H, d), 4.52 (1H, s), 6.07 (1H, ddt), 6.54 (1H, t), 6.77 (1H, s), 7.46 (2H, d), 7.98 (2H, d), 8.91 (1H, s).

### EXAMPLE 59e

¹H NMR (400.132 MHz, DMSO-d₆) δ 0.53-0.49 (2H, m), 0.60-0.56 (2H, m), 1.20 (3H, d), 1.59-1.52 (2H, m), 1.80-1.77 (2H, m), 2.11 (3H, s), 2.32 (3H, s), 3.18-3.14 (1H, m), 3.22 (2H, d), 3.47 (1H, ddd), 3.62 (1H, dd), 3.76 (1H, d), 3.97 (1H, dd), 4.17 (1H, d), 4.52 (1H, s), 5.37 (1H, s), 6.33 (1H, t), 6.76 (1H, s), 7.43 (2H, d), 7.96 (2H, d), 8.80 (1H, s);

### EXAMPLE 59f

¹H NMR (400.132 MHz, DMSO-d₆) δ 1.07 (3H, t), 1.21 (3H, d), 1.59-1.52 (2H, m), 1.80-1.77 (2H, m), 2.11 (3H, s), 2.32 (3H, s), 3.22-3.09 (3H, m), 3.47 (1H, ddd), 3.62 (1H, dd), 3.76 (1H, d), 3.97 (1H, dd), 4.17 (1H, d), 4.53 (1H, s), 6.17 (1H, t), 6.75 (1H, s), 7.44 (2H, d), 7.95 (2H, d), 8.64 (1H, s);

### EXAMPLE 59g

¹H NMR (400.132 MHz, DMSO-d₆) δ 1.21 (3H, d), 1.59-1.53 (2H, m), 1.81-1.78 (2H, m), 2.12 (3H, s), 2.33 (3H, s), 3.23-3.15 (1H, m), 3.48 (1H, ddd), 3.63 (1H, dd), 3.76 (1H, d), 3.79 (3H, s), 3.97 (1H, dd), 4.18 (1H, d), 4.53 (1H, s), 6.77 (1H, s), 7.39 (1H, s), 7.49 (2H, d), 7.77 (1H, s), 7.99 (2H, d), 8.40 (1H, s), 8.82 (1H, s).

The preparation of phenyl N-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]carbamate is described below.

### Phenyl N-[4-[4-[1-[(3,5-dimethyl-1,2-oxazol-4-yl)sulfonyl]cyclopropyl]-6-[(3s)-3-methylmorpholin-4-yl]pyrimidin-2-yl]phenyl]carbamate

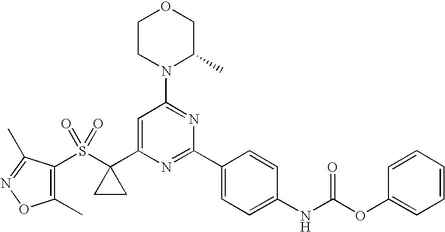

4-[4-[1-[(3,5-Dimethyl-1,2-oxazol-4-yl)sulfonyl]cyclopropyl]-6-[(3S)-3-methylmorpholin-4-yl]pyrimidin-2-yl]aniline (as the hydrochloride salt) (1.94 g, 3.83 mmol) and sodium bicarbonate (1.610 g, 19.17 mmol) were added to DCM (60 mL) and stirred for 10 minutes. Phenyl chloroformate (0.625 mL, 4.98 mmol) was added slowly and the reaction was stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. DCM followed by diethyl ether were added and the solvent was slowly removed until a solid was obtained. The solid was collected by filtration and dried under vacuum to give the desired material as as a white solid (1.89 g).

NMR Spectrum: ¹H NMR (400.132 MHz, DMSO-d₆) δ 1.21 (3H, d), 1.60-1.52 (2H, m), 1.81-1.78 (2H, m), 2.11 (3H, s), 2.33 (3H, s), 3.19 (1H, ddd), 3.48 (1H, ddd), 3.63 (1H, dd), 3.76 (1H, d), 3.97 (1H, dd), 4.19 (1H, d), 4.54 (1H, s), 6.80 (1H, s), 7.30-7.24 (3H, m), 7.45 (2H, t), 7.57 (2H, d), 8.04 (2H, d), 10.42 (1H, s).

LCMS Spectrum: m/z (ESI+) (M+H)+=590; HPLC tR=2.86 min

### 4-[4-[1-[(3,5-Dimethyl-1,2-oxazol-4-yl)sulfonyl]cyclopropyl]-6-[(3S)-3-methylmorpholin-4-yl]pyrimidin-2-yl]aniline

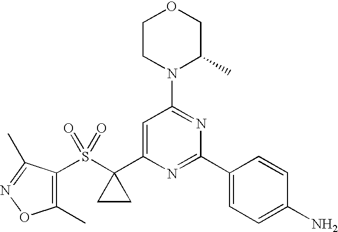

tert-Butyl N-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]carbamate (2.6 g, 4.56 mmol) was added to 6N hydrogen chloride in propan-2-ol (40 mL) and stirred for 2 hours at RT. The crude solution was triturated with diethyl ether to give the desired material (as the hydrochloride salt) as a yellow solid (1.96 g). The material was used without further purification.

NMR Spectrum: ¹H NMR (400.132 MHz, DMSO-d₆) δ 1.24 (3H, d), 1.69-1.58 (2H, m), 1.84-1.82 (2H, m), 2.09 (3H, s), 2.38 (3H, s), 3.30-3.24 (1H, m), 3.50-3.45 (1H, m), 3.62 (1H, dd), 3.81-3.75 (1H, m), 3.99 (1H, dd), 4.28 (1H, s), 4.60 (1H, s), 6.93 (1H, s), 7.23-7.13 (2H, m), 8.05 (2H, d).

LCMS Spectrum: m/z (ESI+) (M+H)+=470; HPLC tR=1.88 min

### tert-Butyl N-[4-[4-[1-[(3,5-dimethyl-1,2-oxazol-4-yl)sulfonyl]cyclopropyl]-6-[(3S)-3-methylmorpholin-4-yl]pyrimidin-2-yl]phenyl]carbamate

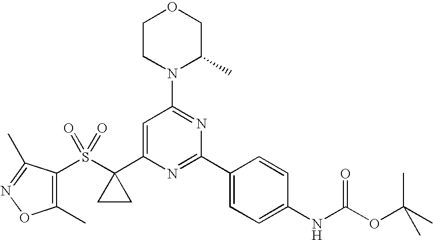

Sodium hydride (2.177 g, 45.36 mmol) and 1,2-dibromoethane (2.61 mL, 30.24 mmol) in DMF (70 mL) were added rapidly to a solution of tert-butyl N-[4-[4-[(3,5-dimethyl-1,2-oxazol-4-yl)sulfonylmethyl]-6-[(3S)-3-methylmorpholin-4-yl]pyrimidin-2-yl]phenyl]carbamate (4.11 g, 7.56 mmol) in DMF (70 mL) at RT. The resulting suspension was stirred at RT for 1 hour. Additional sodium hydride (1.1 g, 22.68 mmol) and 1,2-dibromoethane (1.305 mL, 15.12 mmol) were added and the reaction was stirred at RT for 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to afford a yellow foam. This was dissolved in 40% ethyl acetate in isohexane and stirred to give the desired material as a white solid which was collected by filtration (2.60 g).

NMR Spectrum: ¹H NMR (400.132 MHz, DMSO-d₆) δ 1.21 (3H, d), 1.50 (9H, s), 1.59-1.53 (2H, m), 1.81-1.77 (2H, m), 2.11 (3H, s), 2.32 (3H, s), 3.18 (1H, ddd), 3.47 (1H, ddd), 3.62 (1H, dd), 3.76 (1H, d), 3.97 (1H, dd), 4.18 (1H, d), 4.53 (1H, s), 6.78 (1H, s), 7.51 (2H, d), 7.97 (2H, d), 9.54 (1H, s).

LCMS Spectrum: m/z (ESI+) (M+H)+=570; HPLC tR=2.99 min

### tert-Butyl N-[4-[4-[(3,5-dimethyl-1,2-oxazol-4-yl)sulfonylmethyl]-6-[(3S)-3-methylmorpholin-4-yl]pyrimidin-2-yl]phenyl]carbamate

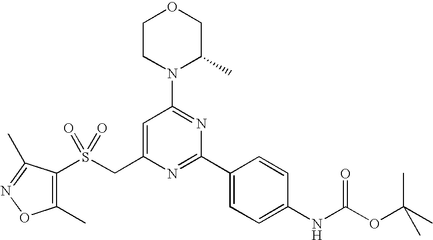

3,5-dimethylisoxazole-4-sulfinic acid, sodium salt (1.443 g, 7.84 mmol) and tert-Butyl N-[4-[4-(iodomethyl)-6-[(3S)-3-methylmorpholin-4-yl]pyrimidin-2-yl]phenyl]carbamate (4 g, 7.84 mmol) were dissolved in DMF (50 mL) and stirred for 1 hour at RT. The solvent was evaporated to afford a yellow solid, this was quenched with water (50 mL) and extracted with DCM (2×75 mL), dried and solvent evaporated to afford an orange gum, this was rapidly stirred with diethyl ether (100 mL) to afford a solid which was collected by filtration and dried under vacuum to give the desired material as an off white solid (4.11 g).

NMR Spectrum: ¹H NMR (400.132 MHz, DMSO-d₆) δ 1.22 (3H, d), 1.50 (9H, s), 2.19 (3H, s), 2.33 (3H, s), 3.20 (1H, ddd), 3.50 (1H, ddd), 3.65 (1H, dd), 3.78 (1H, d), 3.98 (1H, dd), 4.15 (1H, d), 4.44 (1H, s), 4.69 (2H, s), 6.79 (1H, s), 7.51 (2H, d), 7.97 (2H, d), 9.55 (1H, s).

LCMS Spectrum: m/z (ESI+) (M+H)+=544; HPLC tR=2.76 min

### 3,5-Dimethylisoxazole-4-sulfinic acid, sodium salt

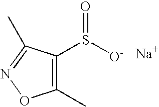

A solution of sodium sulfite (5.03 g, 39.87 mmol) in water (50 mL) was stirred at RT for 10 minutes. Sodium bicarbonate (6.70 g, 79.74 mmol) was added and the resulting solution was stirred at 50° C. for 10 minutes. 3,5-Dimethylisoxazole-4-sulfonyl chloride (7.8 g, 39.87 mmol) was added portion-wise to the solution and was stirred at 50° C. for 2 hours. The reaction mixture was evaporated to dryness and re-dissolved in methanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (7.16 g).

NMR Spectrum: ¹H NMR (400.132 MHz, DMSO-d₆) δ 2.24 (3H, s), 2.39 (3H, s).

### tert-Butyl N-[4-[4-(iodomethyl)-6-[(3S)-3-methylmorpholin-4-yl]pyrimidin-2-yl]phenyl]carbamate

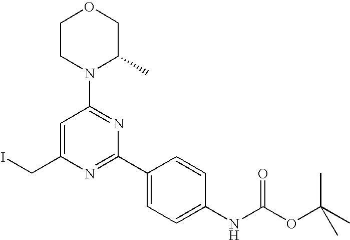

[6-[(3S)-3-Methylmorpholin-4-yl]-2-[4-[(2-methylpropan-2-yl)oxycarbonylamino]phenyl]pyrimidin-4-yl]methyl methanesulfonate (27 g, 56.42 mmol) and lithium iodide (4.33 mL, 112.84 mmol) were added to dioxane (250 mL) and heated at 60° C. for 1 hour and then at RT overnight. The solvent was evaporated to dryness, the reaction mixture was quenched with a saturated aqueous solution of ammonium chloride (100 mL) and extracted with DCM (3×75 mL). The organic extracts were then flushed through a two inch silica plug, eluting with ethyl acetate, to give a brown foam. This was rapidly dissolved in diethyl ether and stirred to afford the desired material as a white solid (25.2 g).

NMR Spectrum: ¹H NMR (400.132 MHz, DMSO-d₆) δ 1.22 (3H, d), 1.50 (9H, s), 3.19 (1H, ddd), 3.49 (1H, ddd), 3.64 (1H, dd), 3.76 (1H, d), 3.97 (1H, dd), 4.16 (1H, d), 4.39 (2H, s), 4.48 (1H, s), 6.80 (1H, s), 7.55 (2H, d), 8.22 (2H, d), 9.53 (1H, s).

LCMS Spectrum: m/z (ESI+) (M+H)+=511; HPLC tR=2.85 min

The preparation of [6-[(3S)-3-methylmorpholin-4-yl]-2-[4-[(2-methylpropan-2-yl)oxycarbonylamino]phenyl]pyrimidin-4-yl]methyl methanesulfonate was described earlier.

### EXAMPLE 60

### 1-[4-[4-[1-(2,5-Difluorophenyl)sulfonylcyclopropyl]-6-[(3S)-3-methylmorpholin-4-yl]pyrimidin-2-yl]phenyl]-3-methylurea

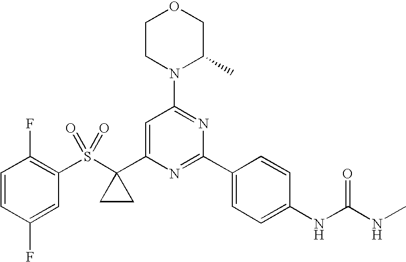

Phenyl N-[4-[4-[1-(2,5-difluorophenyl)sulfonylcyclopropyl]-6-[(3S)-3-methylmorpholin-4-yl]pyrimidin-2-yl]phenyl]carbamate (0.15 g, 0.25 mmol) and methylamine (2M in THF, 1.236 mL, 2.47 mmol) were added to dioxane (10 mL) and stirred overnight. The reaction was evaporated to dryness and was purified by preparative HPLC, eluting with decreasingly polar mixtures of water (containing 1% ammonia) and acetonitrile, to give the desired material as a white solid (0.113 g).

NMR Spectrum: ¹H NMR (400.132 MHz, DMSO-d₆) δ 1.19 (3H, d), 1.72-1.70 (2H, m), 1.96-1.91 (2H, m), 2.66 (3H, d), 3.16 (1H, ddd), 3.46 (1H, ddd), 3.61 (1H, dd), 3.74 (1H, d), 3.95 (1H, dd), 4.16 (1H, d), 4.48 (1H, s), 6.05 (1H, q), 6.68 (1H, s), 7.37 (2H, d), 7.54-7.50 (1H, m), 7.69-7.57 (2H, m), 7.73 (2H, d), 8.70 (1H, s);

LCMS Spectrum: m/z (ESI+) (M+H)+=544; HPLC tR=2.30 min

The following compounds were prepared in an analogous fashion from phenyl N-[4-[4-[1-(2,5-difluorophenyl)sulfonylcyclopropyl]-6-[(3S)-3-methylmorpholin-4-yl]pyrimidin-2-yl]phenyl]carbamate and the appropriate amine.

| | | | | Reten- |
|---|---|---|---|---|
| | | | | tion |
| | | | LCMS | time |
| Example | Structure | NAME | MH+ | (min) |
| 60a | 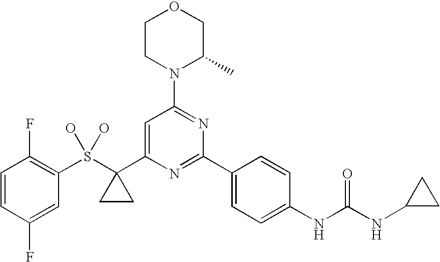 | 3-cyclopropyl-1-[4-[4-[1-(2,5-difluorophenyl)sulfonylcyclopropyl]-6-[(3S)-3-methylmorpholin-4-yl]pyrimidin-2-yl]phenyl]urea | 570 | 2.51 |
| 60b | 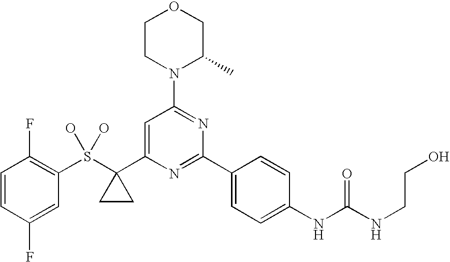 | 1-[4-[4-[1-(2,5-difluorophenyl)sulfonylcyclopropyl]-6-[(3S)-3-methylmorpholin-4-yl]pyrimidin-2-yl]phenyl]-3-(2-hydroxyethyl)urea | 574 | 2.11 |
| 60c | 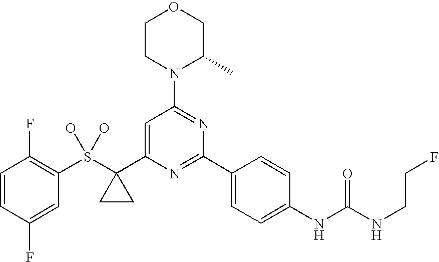 | 1-[4-[4-[1-(2,5-difluorophenyl)sulfonylcyclopropyl]-6-[(3S)-3-methylmorpholin-4-yl]pyrimidin-2-yl]phenyl]-3-(2-fluoroethyl)urea | 576 | 2.48 |
| 60d | 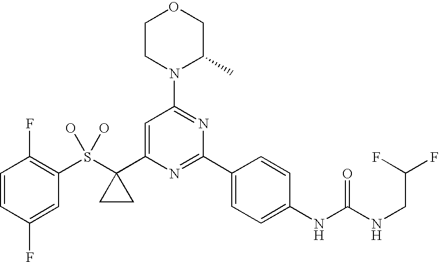 | 3-(2,2-difluoroethyl)-1-[4-[4-[1-(2,5-difluorophenyl)sulfonylcyclopropyl]-6-[(3S)-3-methylmorpholin-4-yl]pyrimidin-2-yl]phenyl]urea | 594 | 2.61 |
| 60e | 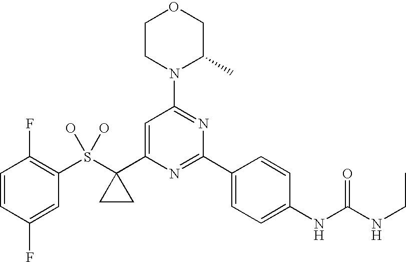 | 3-[4-[4-[1-(2,5-difluorophenyl)sulfonylcyclopropyl]-6-[(3S)-3-methylmorpholin-4-yl]pyrimidin-2-yl]phenyl]-1-ethylurea | 558 | 2.48 |
| 60f | 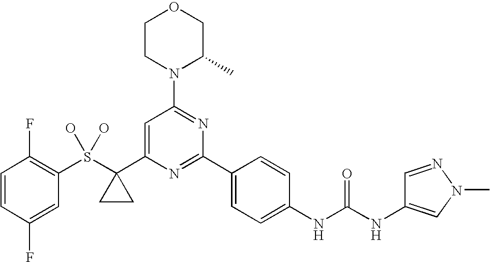 | 1-[4-[4-[1-(2,5-difluorophenyl)sulfonylcyclopropyl]-6-[(3S)-3-methylmorpholin-4-yl]pyrimidin-2-yl]phenyl]-3-(1-methylpyrazol-4-yl)urea | 610 | 2.46 |

### EXAMPLE 60a

¹H NMR (400.132 MHz, DMSO-d₆) δ 0.44-0.40 (2H, m), 0.67-0.62 (2H, m), 1.19 (3H, d), 1.74-1.68 (2H, m), 1.96-1.90 (2H, m), 2.58-2.53 (1H, m), 3.16 (1H, ddd), 3.46 (1H, ddd), 3.61 (1H, dd), 3.74 (1H, d), 3.95 (1H, dd), 4.16 (1H, d), 4.48 (1H, s), 6.41 (1H, s), 6.68 (1H, s), 7.38 (2H, d), 7.54-7.50 (1H, m), 7.69-7.57 (2H, m), 7.73 (2H, d), 8.50 (1H, s);

### EXAMPLE 60b

¹H NMR (400.132 MHz, DMSO-d₆) δ 1.19 (3H, d), 1.74-1.68 (2H, m), 1.98-1.89 (2H, m), 2.09 (3H, s), 3.20-3.12 (3H, m), 3.49-3.42 (3H, m), 3.61 (1H, dd)), 3.74 (1H, d), 3.95 (1H, dd), 4.16 (1H, d), 4.48 (1H, s), 4.73 (1H, t), 6.24 (1H, t), 6.68 (1H, s), 7.36 (2H, d), 7.54-7.50 (1H, m), 7.69-7.57 (2H, m), 7.73 (2H, d), 8.77 (1H, s);

### EXAMPLE 60c

¹H NMR (400.132 MHz, DMSO-d₆) δ 1.19 (3H, d), 1.74-1.68 (2H, m), 1.96-1.90 (2H, m), 3.16 (1H, ddd), 3.48-3.36 (3H, m), 3.61 (1H, dd), 3.74 (1H, d), 3.95 (1H, dd), 4.16 (1H, d), 4.47 (2H, dt), 4.48 (1H, s), 6.41 (1H, t), 6.68 (1H, s), 7.37 (2H, d), 7.54-7.50 (1H, m), 7.69-7.57 (2H, m), 7.74 (2H, d), 8.77 (1H, s);

### EXAMPLE 60d

¹H NMR (400.132 MHz, DMSO-d₆) δ 1.19 (3H, d), 1.74-1.68 (2H, m), 1.96-1.91 (2H, m), 3.16 (1H, ddd), 3.63-3.43 (4H, m), 3.74 (1H, d), 3.96 (1H, dd), 4.16 (1H, d), 4.48 (1H, s), 6.07 (1H, ddt), 6.51 (1H, t), 6.69 (1H, s), 7.38 (2H, d), 7.54-7.50 (1H, m), 7.69-7.57 (2H, m), 7.75 (2H, d), 8.89 (1H, s);

### EXAMPLE 60e

¹H NMR (400.132 MHz, DMSO-d₆) δ 1.07 (3H, t), 1.19 (3H, d), 1.72-1.69 (2H, m), 1.95-1.91 (2H, m), 3.19-3.09 (3H, m), 3.45 (1H, ddd), 3.61 (1H, dd), 3.74 (1H, d), 3.95 (1H, dd), 4.16 (1H, d), 4.48 (1H, s), 6.14 (1H, t), 6.68 (1H, s), 7.36 (2H, d), 7.54-7.50 (1H, m), 7.69-7.57 (2H, m), 7.72 (2H, d), 8.62 (1H, s);

### EXAMPLE 60f

¹H NMR (400.132 MHz, DMSO-d₆) δ 1.19 (3H, d), 1.74-1.68 (2H, m), 1.96-1.92 (2H, m), 3.17 (1H, ddd), 3.46 (1H, ddd), 3.61 (1H, dd), 3.75 (1H, d), 3.79 (3H, s), 3.96 (1H, dd), 4.17 (1H, d), 4.48 (1H, s), 6.69 (1H, s), 7.38 (1H, s), 7.42 (2H, d), 7.55-7.51 (1H, m), 7.70-7.58 (2H, m), 7.78-7.75 (3H, m), 8.36 (1H, s), 8.80 (1H, s);

The preparation of phenyl N-[4-[4-[1-(2,5-difluorophenyl)sulfonylcyclopropyl]-6-[(3S)-3-methylmorpholin-4-yl]pyrimidin-2-yl]phenyl]carbamate is described below.

### Phenyl N-[4-[4-[1-(2,5-difluorophenyl)sulfonylcyclopropyl]-6-[(3S)-3-methylmorpholin-4-yl]pyrimidin-2-yl]phenyl]carbamate

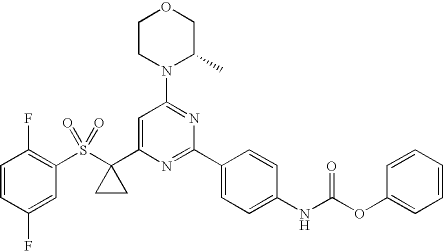

4-[4-[1-(2,5-Difluorophenyl)sulfonylcyclopropyl]-6-[(3S)-3-methylmorpholin-4-yl]pyrimidin-2-yl]aniline (as the hydrochloride salt) (2.54 g, 4.86 mmol) and sodium bicarbonate (2.04 g, 24.28 mmol) were added to DCM (60 mL) and stirred for 10 minutes. Phenyl chloroformate (0.792 mL, 6.31 mmol) was added slowly and the reaction was stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. The solid was passed through a plug of silica, eluting with ethyl acetate, to give a solid that was further purified by trituration with diethyl ether to give the desired material as a white solid (2.20 g).

NMR Spectrum: ¹H NMR (400.132 MHz, DMSO-d₆) δ 1.20 (3H, d), 1.73-1.69 (2H, m), 1.98-1.91 (2H, m), 3.17 (1H, ddd), 3.51-3.44 (1H, m), 3.61 (1H, dd), 3.75 (1H, d), 3.95 (1H, d), 4.18 (1H, d), 4.49 (1H, s), 6.72 (1H, s), 7.30-7.24 (3H, m), 7.45 (2H, t), 7.55-7.50 (3H, m), 7.69-7.57 (2H, m), 7.83 (2H, d), 10.39 (1H, s);

LCMS Spectrum: m/z (ESI+) (M+H)+=607; HPLC tR=3.15 min

### 4-[4-[1-(2,5-Difluorophenyl)sulfonylcyclopropyl]-6-[(3S)-3-methylmorpholin-4-yl]pyrimidin-2-yl]aniline

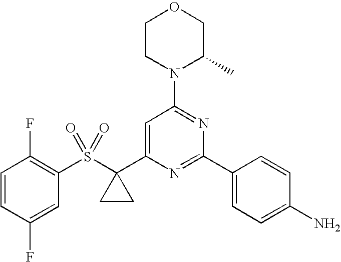

tert-Butyl N-[4-[4-[1-(2,5-difluorophenyl)sulfonylcyclopropyl]-6-[(3S)-3-methylmorpholin-4-yl]pyrimidin-2-yl]phenyl]carbamate (2.85 g, 4.86 mmol) was added to 6N hydrogen chloride in propan-2-ol (30 mL) and stirred for 2 hours at RT. The crude solution was triturated with diethyl ether to give the desired material (as a hydrochloride salt) as a yellow solid (2.4 g, 94 %). The material was used without further purification.

NMR Spectrum: ¹H NMR (400.132 MHz, DMSO-d₆) δ 1.20 (3H, d), 1.78-1.71 (2H, m), 1.97-1.93 (2H, m), 3.23 (1H, ddd), 3.45 (1H, ddd), 3.59 (1H, dd), 3.82-3.73 (1H, m), 3.96 (1H, dd), 4.29-4.18 (1H, m), 4.59-4.47 (1H, m), 6.81 (1H, s), 7.25 (2H, d), 7.63-7.54 (2H, m), 7.72-7.66 (1H, m), 7.95 (2H, d);

LCMS Spectrum: m/z (ESI+) (M+H)+=487; HPLC tR=2.52 min

### tert-Butyl N-[4-[4-[1-(2,5-difluorophenyl)sulfonylcyclopropyl]-6-[(3S)-3-methylmorpholin-4-yl]pyrimidin-2-yl]phenyl]carbamate

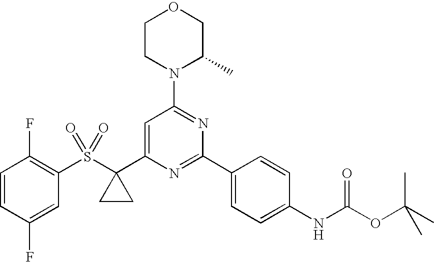

Sodium hydride (1.38 g, 28.56 mmol) was added rapidly to a solution of tert-butyl N-[4-[4-[(2,5-difluorophenyl)sulfonylmethyl]-6-[(3S)-3-methylmorpholin-4-yl]pyrimidin-2-yl]phenyl]carbamate (4.0 g, 7.14 mmol) in DMF (70 mL) and the mixture stirred at 40° C. for 10 minutes before the slow addition 1,2-dibromoethane (2.459 mL, 28.54 mmol) in DMF (70 mL) at RT. The resulting suspension was stirred at 40° C. for 1 hour. Additional sodium hydride (0.69 g, 14.26) and 1,2-dibromoethane (1.23 mL, 14.26 mmol) were added and the reaction was stirred at RT for 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to give a yellow foam. This was dissolved in 40% ethyl acetate in isohexane and upon stirring the desired material precipitated out as a white solid (2.85 g).

NMR Spectrum: ¹H NMR (400.132 MHz, DMSO-d₆) δ 1.19 (3H, d), 1.49 (9H, s), 1.74-1.68 (2H, m), 1.96-1.92 (2H, m), 3.16 (1H, ddd), 3.46 (1H, ddd), 3.61 (1H, dd), 3.74 (1H, d), 3.95 (1H, dd), 4.17 (1H, d), 4.48 (1H, s), 6.70 (1H, s), 7.44 (2H, d), 7.54-7.50 (1H, m), 7.69-7.57 (2H, m), 7.76 (2H, d), 9.50 (1H, s);

LCMS Spectrum: m/z (ESI+) (M+H)+=587; HPLC tR=2.94 min

### tert-Butyl N-[4-[4-[(2,5-difluorophenyl)sulfonylmethyl]-6-[(3S)-3-methylmorpholin-4-yl]pyrimidin-2-yl]phenyl]carbamate

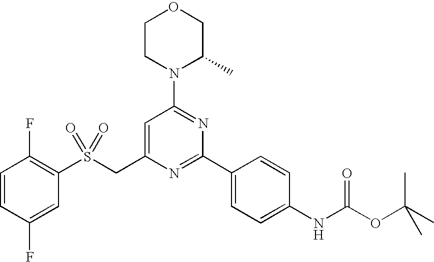

Sodium 2,5-difluorobenzenesulfonate (2.117 g, 9.80 mmol) and tert-butyl N-[4-[4-(iodomethyl)-6-[(3S)-3-methylmorpholin-4-yl]pyrimidin-2-yl]phenyl]carbamate (5 g, 9.80 mmol) were dissolved in DMF (50 mL) and stirred for 1 hour at RT. The solvent was evaporated and the residue partitioned between water (50 mL) and DCM (75 mL). The layers were separated, the aqueous layer further extracted with DCM (75 mL) and the combined organics passed through a plug of silica, eluting with ethyl acetate, to give a yellow solid. This material was triturated with diethyl ether to give the desired material as a white solid (4.90 g).

NMR Spectrum: ¹H NMR (400.132 MHz, DMSO-d₆) δ 1.21 (3H, d), 1.49 (9H, s), 3.19 (1H, ddd), 3.48 (1H, ddd), 3.63 (1H, dd), 3.76 (1H, d), 3.97 (1H, dd), 4.13 (1H, d), 4.41 (1H, s), 4.82 (2H, s), 6.78 (1H, s), 7.44-7.42 (3H, m), 7.75-7.68 (4H, m), 9.50 (1H, s);

LCMS Spectrum: m/z (ESI+) (M+H)+=561; HPLC tR=2.95 min

### 2,5-Difluorobenzenesulfinic acid, sodium salt

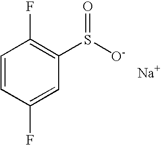

A solution of sodium sulfite (29.6 g, 235.18 mmol) in water (200 mL) was stirred at RT for 10 minutes. Sodium bicarbonate (39.5 g, 470.36 mmol) was added to the stirred solution. The resulting solution was stirred at 50° C. for 10 minutes. 2,5-difluorobenzene-1-sulfonyl chloride (50 g, 235 mmol) was added portionwise and the solution stirred at 50° C. for 2 hours. The reaction mixture was evaporated to dryness and redissolved in ethanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (43.0 g).

NMR Spectrum: ¹H NMR (400.132 MHz, DMSO-d₆) δ 7.09-7.05 (2H, m), 7.36-7.32 (1H, m).

The preparation of tert-butyl N-[4-[4-(iodomethyl)-6-[(3S)-3-methylmorpholin-4-yl]pyrimidin-2-yl]phenyl]carbamate was described earlier.

### EXAMPLE 61

### 1-[4-[4-[1-(5-Fluoro-2-methylphenyl)sulfonylcyclopropyl]-6-[(3S)-3-methylmorpholin-4-yl]pyrimidin-2-yl]phenyl]-3-methylurea

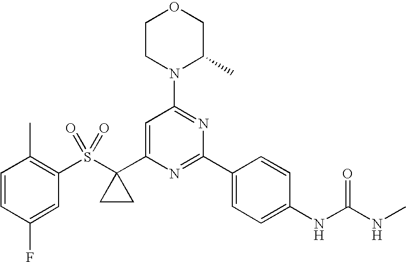

Phenyl N-[4-[4-[1-(5-fluoro-2-methylphenyl)sulfonylcyclopropyl]-6-[(3S)-3-methylmorpholin-4-yl]pyrimidin-2-yl]phenyl]carbamate (0.15 g, 0.25 mmol) and methylamine (2M in THF, 1.244 mL, 2.49 mmol) were added to dioxane (10 mL) and stirred overnight. The reaction was evaporated to dryness and was purified by preparative HPLC, eluting with decreasingly polar mixtures of water (containing 1% ammonia) and acetonitrile, to give the desired material as a white solid (0.130 g).

NMR Spectrum: ¹H NMR (400.132 MHz, DMSO-d₆) δ 1.16 (3H, d), 1.70-1.64 (2H, m), 1.90-1.87 (2H, m), 2.41 (3H, s), 2.66 (3H, d), 3.12 (1H, ddd), 3.44 (1H, ddd), 3.60 (1H, dd), 3.74 (1H, d), 3.95 (1H, dd), 4.10 (1H, d), 4.43 (1H, s), 6.05 (1H, q), 6.64 (1H, s), 7.42-7.38 (4H, m), 7.62 (1H, d), 7.86 (2H, d), 8.71 (1H, s).

LCMS Spectrum: m/z (ESI+) (M+H)+=540; HPLC tR=2.46 min

The following compounds were prepared in an analogous fashion from phenyl N-[4-[4-[1-(5-fluoro-2-methylphenyl)sulfonylcyclopropyl]-6-[(3S)-3-methylmorpholin-4-yl]pyrimidin-2-yl]phenyl]carbamate and the appropriate amine.

| | | | | Reten- |
|---|---|---|---|---|
| | | | | tion |
| | | | LCMS | time |
| Example | Structure | NAME | MH+ | (min) |
| 61a | 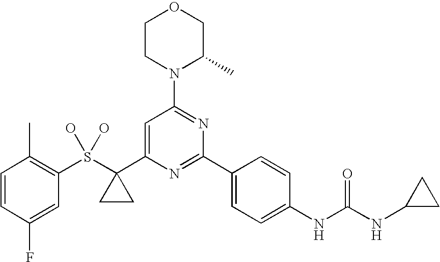 | 3-cyclopropyl-1-[4-[4-[1-(5-fluoro-2-methylphenyl)sulfonylcyclopropyl]-6-[(3S)-3-methylmorphohn-4-yl]pyrimidin-2-yl]phenyl]urea | 566 | 2.65 |
| 61b | 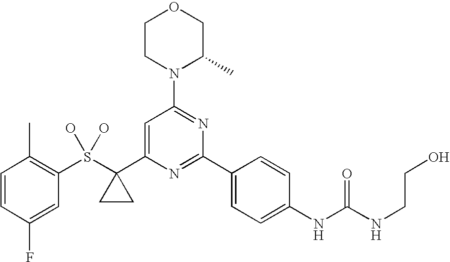 | 1-[4-[4-[1-(5-fluoro-2-methylphenyl)sulfonylcyclopropyl]-6-[(3S)-3-methylmorpholin-4-yl]pyrimidin-2-yl]phenyl]-3-(2-hydroxyethyl)urea | 570 | 2.28 |
| 61c | 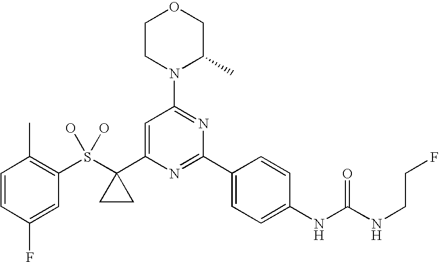 | 3-(2-fluoroethyl)-1-[4-[4-[1-(5-fluoro-2-methylphenyl)sulfonylcyclopropyl]-6-[(3S)-3-methylmorpholin-4-yl]pyrimidin-2-yl]phenyl]urea | 572 | 2.48 |
| 61d | 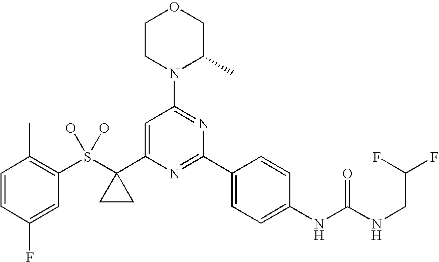 | 3-(2,2-difluoroethyl)-1-[4-[4-[1-(5-fluoro-2-methylphenyl)sulfonylcyclopropyl]-6-[(3S)-3-methylmorphohn-4-yl]pyrimidin-2-yl]phenyl]urea | 590 | 2.74 |
| 61e | 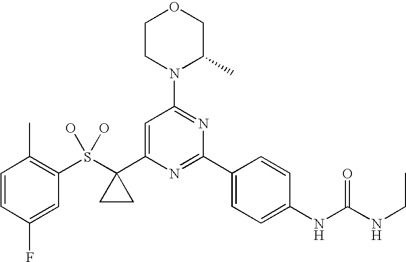 | 1-ethyl-3-[4-[4-[1-(5-fluoro-2-methylphenyl)sulfonylcyclopropyl]-6-[(3S)-3-methylmorpholin-4-yl]pyrimidin-2-yl]phenyl]urea | 554 | 2.54 |
| 61f | 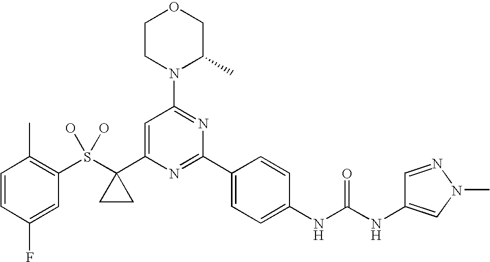 | 1-[4-[4-[1-(5-fluoro-2-methylphenyl)sulfonylcyclopropyl]-6-[(3S)-3-methylmorpholin-4-yl]pyrimidin-2-yl]phenyl]-3-(1-methylpyrazol-4-yl)urea | 606 | 2.40 |

### EXAMPLE 61a

¹H NMR (400.132 MHz, DMSO-d₆) δ 0.44-0.40 (2H, m), 0.67-0.63 (2H, m), 1.16 (3H, d), 1.73-1.64 (2H, m), 1.90-1.87 (2H, m), 2.41 (3H, s), 2.59-2.53 (1H, m), 3.12 (1H, ddd), 3.45 (1H, ddd), 3.60 (1H, dd), 3.74 (1H, d), 3.95 (1H, dd), 4.10 (1H, d), 4.43 (1H, s), 6.42 (1H, s), 6.64 (1H, s), 7.42-7.38 (4H, m), 7.63-7.61 (1H, m), 7.86 (2H, d), 8.50 (1H, s).

### EXAMPLE 61b

¹H NMR (400.132 MHz, DMSO-d₆) δ 1.16 (3H, d), 1.70-1.64 (2H, m), 1.90-1.87 (2H, m), 2.41 (3H, s), 3.20-3.09 (3H, m), 3.48-3.41 (3H, m), 3.60 (1H, dd), 3.74 (1H, d), 3.95 (1H, dd), 4.10 (1H, d), 4.43 (1H, s), 4.73 (1H, t), 6.24 (1H, t), 6.64 (1H, s), 7.40-7.38 (4H, m), 7.63-7.60 (1H, m), 7.86 (2H, d), 8.77 (1H, s);

### EXAMPLE 61c

¹H NMR (400.132 MHz, DMSO-d₆) δ 1.16 (3H, d), 1.70-1.64 (2H, m), 1.91-1.87 (2H, m), 2.41 (3H, s), 3.13 (1H, ddd), 3.47-3.37 (3H, m), 3.60 (1H, dd), 3.74 (1H, d), 3.95 (1H, dd), 4.10 (1H, d), 4.47-4.40 (1H, m), 4.47 (2H, dt), 6.42 (1H, t), 6.64 (1H, s), 7.41-7.38 (4H, m), 7.61 (1H, d), 7.87 (2H, d), 8.78 (1H, s).

### EXAMPLE 61d

¹H NMR (400.132 MHz, DMSO-d₆) δ 1.16 (3H, d), 1.70-1.64 (2H, m), 1.90-1.87 (2H, m), 2.41 (3H, s), 3.12 (1H, ddd), 3.62-3.41 (4H, m), 3.74 (1H, d), 3.95 (1H, dd), 4.10 (1H, d), 4.44 (1H, s), 6.08 (1H, ddt), 6.52 (1H, t), 6.65 (1H, s), 7.42-7.38 (4H, m), 7.62

### EXAMPLE 61e

¹H NMR (400.132 MHz, DMSO-d₆) δ 1.07 (3H, t), 1.16 (3H, d), 1.70-1.65 (2H, m), 1.90-1.85 (2H, m), 2.4 (S, 3H), 3.15-3.09 (3H, m), 3.44 (1H, ddd), 3.60 (1H, dd), 3.74 (1H, d), 3.95 (1H, dd), 4.10 (1H, d), 4.43 (1H, s), 6.14 (1H, t), 6.64 (1H, s), 7.39 (4H, d), 7.62 (1H, d), 7.86 (2H, d), 8.63 (1H, s).

### EXAMPLE 61f

¹H NMR (400.132 MHz, DMSO-d₆) δ 1.17 (3H, d), 1.71-1.65 (2H, m), 1.90-1.88 (2H, m), 2.41 (3H, s), 3.13 (1H, ddd), 3.45 (1H, ddd), 3.60 (1H, dd), 3.74 (1H, d), 3.77 (3H, s), 3.95 (1H, ddd), 4.43 (1H, s), 6.65 (1H, s), 7.41-7.39 (4H, m), 7.45 (2H, d), 7.62 (1H, d), 7.77 (1H, s), 7.89 (2H, d), 8.37 (1H, s), 8.80 (1H, s).

The preparation of phenyl N-[4-[4-[1-(5-fluoro-2-methylphenyl)sulfonylcyclopropyl]-6-[(3S)-3-methylmorpholin-4-yl]pyrimidin-2-yl]phenyl]carbamate is described below.

### Phenyl N-[4-[4-[1-(5-fluoro-2-methylphenyl)sulfonylcyclopropyl]-6-[(3S)-3-methylmorpholin-4-yl]pyrimidin-2-yl]phenyl]carbamate

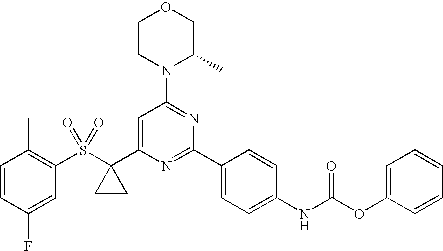

4-[4-[1-(5-Fluoro-2-methylphenyl)sulfonylcyclopropyl]-6-[(3S)-3-methylmorpholin-4-yl]pyrimidin-2-yl]aniline (as the hydrochloride salt) (2.4 g, 4.62 mmol) and sodium bicarbonate (1.942 g, 23.12 mmol) were added to DCM (60 mL) and stirred for 10 minutes. Phenyl chloroformate (0.754 mL, 6.01 mmol) was added slowly and the reaction was stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. To this was added DCM and then diethyl ether, the solvent was slowly removed until the desired material precipitated from the mixture as a white solid (2.05 g).

NMR Spectrum: ¹H NMR (400.132 MHz, DMSO-d₆) δ 1.17 (3H, d), 1.72-1.66 (2H, m), 1.91-1.87 (2H, m), 2.41 (3H, s), 3.14 (1H, ddd), 3.47-3.38 (1H, m), 3.60 (1H, d), 3.74 (1H, d), 3.95 (1H, dd), 4.12 (1H, d), 4.45 (1H, s), 6.68 (1H, s), 7.30-7.24 (3H, m), 7.40 (2H, d), 7.45 (2H, t), 7.54 (2H, d), 7.62 (1H, d), 7.95 (2H, d), 10.39 (1H, s).

LCMS Spectrum: m/z (ESI+) (M+H)+=603; HPLC tR=3.02 min

### 4-[4-[1-(5-Fluoro-2-methylphenyl)sulfonylcyclopropyl]-6-[(3S)-3-methylmorpholin-4-yl]pyrimidin-2-yl]aniline

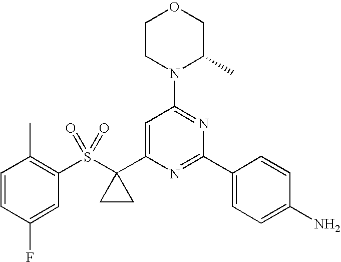

tert-Butyl N-[4-[4-[1-(5-fluoro-2-methylphenyl)sulfonylcyclopropyl]-6-[(3S)-3-methylmorpholin-4-yl]pyrimidin-2-yl]phenyl]carbamate (2.7 g, 4.63 mmol) was added to 6N hydrogen chloride in propan-2-ol (30 mL) and stirred for 2 hours at RT. The crude solution was triturated with diethyl ether to give the desired material (as the hydrochloride salt) as a yellow solid (2.40 g). The material was used without further purification.

NMR Spectrum: ¹H NMR (400.132 MHz, DMSO-d₆) δ 1.18 (3H, d), 1.73-1.69 (2H, m), 1.93-1.90 (2H, m), 2.38 (3H, s), 3.20 (1H, ddd), 3.44 (1H, ddd), 3.58 (1H, dd), 3.75 (1H, d), 3.96 (1H, dd), 4.26-4.12 (1H, m), 4.51 (1H, s), 6.71 (1H, s), 7.17-7.13 (2H, m), 7.45 (2H, d), 7.60 (1H, d), 7.97 (2H, d).

LCMS Spectrum: m/z (ESI+) (M+H)+=483; HPLC tR=2.57 min

### tert-Butyl N-[4-[4-[1-(5-fluoro-2-methylphenyl)sulfonylcyclopropyl]-6-[(3S)-3-methylmorpholin-4-yl]pyrimidin-2-yl]phenyl]carbamate

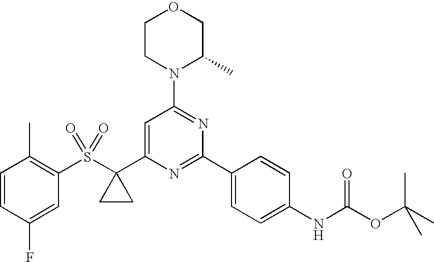

Sodium hydride (1.987 g, 41.39 mmol) was added rapidly to a solution of tert-butyl N-[4-[4-[(5-fluoro-2-methylphenyl)sulfonylmethyl]-6-[(3S)-3-methylmorpholin-4-yl]pyrimidin-2-yl]phenyl]carbamate (3.84 g, 6.90 mmol) in DMF (70 mL) and the mixture stirred at RT for 10 minutes before the slow addition 1,2-dibromoethane (2.38 mL, 27.59 mmol) in DMF (70 mL). The resulting suspension was stirred at RT for 30 minutes. Additional sodium hydride (0.95 g, 20.70 mmol) and 1,2 dibromoethane (1.19 g, 20.70 mmol) were added and the reaction was stirred for a further 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to give a material which was further purified by trituration with 40% ethyl acetate in isohexane to give the desired material as a white solid (2.70 g).

NMR Spectrum: ¹H NMR (400.132 MHz, DMSO-d₆) δ 1.16 (3H, d), 1.49 (9H, s), 1.70-1.64 (2H, m), 1.90-1.87 (2H, m), 2.41 (3H, s), 3.13 (1H, ddd), 3.45 (1H, ddd), 3.60 (1H, dd), 3.74 (1H, d), 3.95 (1H, dd), 4.11 (1H, d), 4.43 (1H, s), 6.66 (1H, s), 7.41-7.38 (2H, m), 7.46 (2H, d), 7.62-7.60 (1H, m), 7.88 (2H, d), 9.50 (1H, s).

LCMS Spectrum: m/z (ESI+) (M+H)+=583; HPLC tR=3.19 min

### tert-Butyl N-[4-[4-[(5-fluoro-2-methylphenyl)sulfonylmethyl]-6-[(3S)-3-methylmorpholin-4-yl]pyrimidin-2-yl]phenyl]carbamate

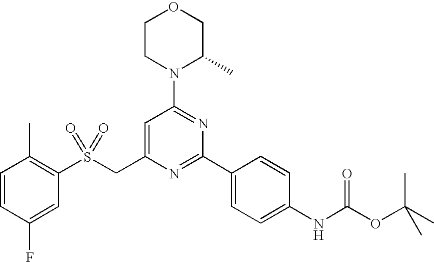

5-Fluoro-2-methylbenzenesulfinic acid, sodium salt (2.079 g, 9.80 mmol) and tert-butyl N-[4-[4-(iodomethyl)-6-[(3S)-3-methylmorpholin-4-yl]pyrimidin-2-yl]phenyl]carbamate (5 g, 9.80 mmol) were dissolved in DMF (50 mL) and stirred for 1 hour at RT The solvent was evaporated and the residue partitioned between water (50 mL) and DCM (75 mL). The layers were separated, the aqueous layer further extracted with DCM (75 mL) and the combined organics dried and solvent evaporated to afford an orange gum which was further purified by trituration with diethyl ether to give the desired material as an off white solid (4.75 g).

NMR Spectrum: ¹H NMR (400.132 MHz, DMSO-d₆) δ 1.20 (3H, d), 1.50 (9H, s), 2.61 (3H, s), 3.17 (1H, ddd), 3.48 (1H, ddd), 3.63 (1H, dd), 3.76 (1H, d), 3.97 (1H, dd), 4.11 (1H, d), 4.40 (1H, s), 4.75 (2H, s), 6.69 (1H, s), 7.50-7.41 (4H, m), 7.56-7.53 (1H, m), 7.82 (2H, d), 9.50 (1H, s).

LCMS Spectrum: m/z (ESI+) (M+H)+=557; HPLC tR=2.91 min

### 5-fluoro-2-methylbenzenesulfinic acid, sodium salt

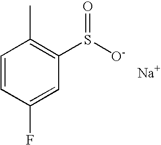

A solution of sodium sulfite (30.2 g, 239.65 mmol) in water (200 mL) was stirred at RT for 10 minutes. Sodium bicarbonate (40.3 g, 479.30 mmol) was added to the stirred solution. The resulting solution was stirred at 50° C. for 10 minutes. 5-Fluoro-2-methyl sulfonyl chloride was added portionwise and the solution stirred at 50° C. for 2 hours. The reaction mixture was evaporated to dryness and redissolved in ethanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (27.0 g).

NMR Spectrum: ¹H NMR (400.132 MHz, DMSO-d₆) δ 2.41 (3H, s), 6.90 (1H, ddd), 7.05 (1H, dd), 7.36 (1H, dd).

The preparation of tert-butyl N-[4-[4-(iodomethyl)-6-[(3S)-3-methylmorpholin-4-yl]pyrimidin-2-yl]phenyl]carbamate was described earlier.

### EXAMPLE 62

### 3-(2-Hydroxyethyl)-1-[4-[4-[(3S)-3-methylmorpholin-4-yl]-6-(1-methylsulfonylcyclobutyl)pyrimidin-2-yl]phenyl]thiourea

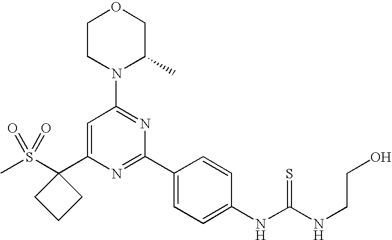

1,1′-Thiocarbonyldiimidazole (57.6 mg, 0.32 mmol) was added to a stirred solution of 4-[4-[(3S)-3-methylmorpholin-4-yl]-6-(1-methylsulfonylcyclobutyl)pyrimidin-2-yl]aniline (100 mg, 0.25 mmol) in THF (1.0 mL) and DCM (2.0 mL) at RT. The resulting solution was stirred for 2 hours. Triethylamine (0.035 mL, 0.25 mmol) and ethanolamine (15.18 mg, 0.25 mmol) were added to the reaction mixture and then stirred at RT for a further 1 hour. The reaction mixture was evaporated to dryness and redissolved in acetonitrile (2.0 mL), filtered and purified by preparative HPLC, eluting with decreasingly polar mixtures of water (containing 1% ammonia) and acetonitrile, to give the desired material as a white solid (73 mg).

NMR Spectrum: ¹H NMR (400.132 MHz, DMSO-d₆) δ 1.25 (3H, d), 1.92 (1H, d), 2.07-2.09 (1H, m), 2.79-2.86 (2H, m), 2.88 (3H, s), 2.87-2.95 (2H, m), 3.18-3.26 (1H, m), 3.47-3.54 (1H, m), 3.57 (3H, s), 3.64-3.67 (1H, m), 3.77 (1H, d), 3.96-4.00 (1H, m), 4.30 (1H, d), 4.63 (1H, d), 4.85 (1H, s), 6.75 (1H, s), 7.61-7.64 (2H, m), 7.90 (1H, s), 8.27-8.30 (2H, m), 9.85 (1H, s)

LCMS Spectrum: m/z (ESI+)(M+H)+=506.55; HPLC tR=2.35 min.

The compounds below were prepared in an analogous fashion from 4-[4-[(3S)-3-methylmorpholin-4-yl]-6-(1-methylsulfonylcyclobutyl)pyrimidin-2-yl]aniline using the appropriate amine.

| | | | | Retention |
|---|---|---|---|---|
| | | | LCMS | time |
| Example | Structure | NAME | MH+ | (min) |
| 62a | 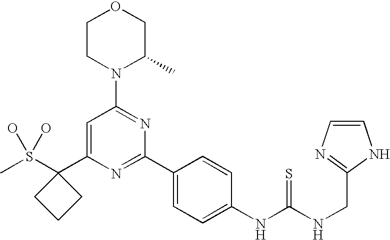 | 3-(1H-imidazol-2-ylmethyl)-1-[4-[4-[(3S)-3-methylmorpholin-4-yl]-6-(1-methylsulfonylcyclobutyl)pyrimi-din-2-yl]phenyl]thiourea | 542.6 | 2.41 |
| 62b | 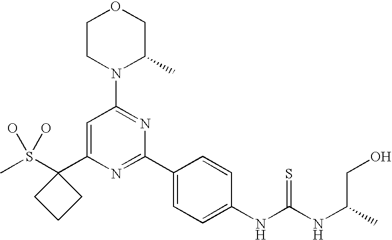 | 3-[(2S)-1-hydroxypropan-2-yl]-1-[4-[4-[(3S)-3-methylmorpholin-4-yl]-6-(1-methylsulfonylcyclobutyl)pyrimi-din-2-yl]phenyl]thiourea | 520.6 | 2.47 |
| 62c | 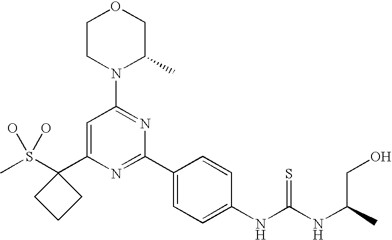 | 3-[(2R)-1-hydroxypropan-2-yl]-1-[4-[4-[(3S)-3-methylmorpholin-4-yl]-6-(1-methylsulfonylcyclobutyl)pyrimi-din-2-yl]phenyl]thiourea | 520.6 | 2.47 |
| 62d | 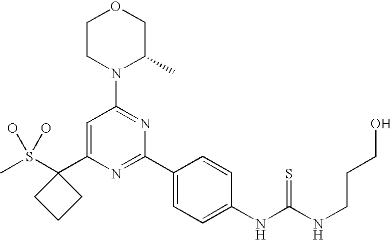 | 3-(3-hydroxypropyl)-1-[4-[4-[(3S)-3-methylmorpholin-4-yl]-6-(1-methylsulfonylcyclobutyl)pyrimi-din-2-yl]phenyl]thiourea | 520.6 | 2.39 |

### EXAMPLE 62a

¹H NMR (400.132 MHz, DMSO-d₆) δ 1.25 (3H, d), 1.90-1.93 (1H, m), 2.07-2.09 (1H, m), 2.79-2.86 (2H, m), 2.88 (3H, s), 2.89-2.95 (2H, m), 3.20-3.26 (1H, m), 3.47-3.54 (1H, m), 3.64-3.67 (1H, m), 3.77 (1H, d), 3.97-4.00 (1H, m), 4.27 (1H, m), 4.60 (1H, s), 4.71 (2H, s), 6.76 (1H, s), 6.88 (1H, s), 7.09 (1H, s), 7.68 (2H, d), 8.24 (1H, s), 8.28-8.32 (2H, m), 10.06 (1H, s).

### EXAMPLE 62b

¹H NMR (400.132 MHz, DMSO-d₆) δ 1.14-1.16 (3H, m), 1.24 (3H, s), 1.90-1.94 (1H, m), 2.07-2.09 (1H, m), 2.79-2.84 (2H, m), 2.88 (3H, s), 2.91-2.95 (2H, m), 3.20-3.26 (1H, m), 3.44-3.54 (3H, m), 3.64-3.67 (1H, m), 3.77 (1H, d), 3.96-4.00 (1H, m), 4.26 (1H, d), 4.38 (1H, s), 4.60 (1H, s), 4.92 (1H, s), 6.75 (1H, s), 7.63-7.66 (2H, m), 7.73-7.75 (1H, m), 8.28 (2H, d), 9.75 (1H, s).

### EXAMPLE 62c

¹H NMR (400.132 MHz, DMSO-d₆) δ 1.14-1.16 (3H, m), 1.24 (3H, s), 1.90-1.94 (1H, m), 2.07-2.09 (1H, m), 2.79-2.84 (2H, m), 2.88 (3H, s), 2.91-2.95 (2H, m), 3.20-3.26 (1H, m), 3.44-3.54 (3H, m), 3.64-3.67 (1H, m), 3.77 (1H, d), 3.96-4.00 (1H, m), 4.26 (1H, d), 4.38 (1H, s), 4.60 (1H, s), 4.92 (1H, s), 6.75 (1H, s), 7.63-7.66 (2H, m), 7.73-7.75 (1H, m), 8.28 (2H, d), 9.75 (1H, s).

### EXAMPLE 62d

¹H NMR (400.132 MHz, DMSO-d₆) δ 1.24-1.25 (3H, m), 1.68-1.75 (2H, m), 1.90-1.93 (1H, m), 2.07-2.09 (1H, m), 2.79-2.86 (2H, m), 2.88 (3H, s), 2.89-2.95 (2H, m), 3.18-3.25 (1H, m), 3.47-3.54 (5H, m), 3.64-3.67 (1H, m), 3.77 (1H, d), 3.96-4.00 (1H, m), 4.26 (1H, d), 4.58 (2H, s), 6.75 (1H, s), 7.55-7.59 (2H, m), 7.93 (1H, s), 8.27-8.30 (2H, m), 9.74 (1H, s).

The preparation of 4-[4-[(3S)-3-methylmorpholin-4-yl]-6-(1-methylsulfonylcyclobutyl)pyrimidin-2-yl]aniline was described earlier.

### EXAMPLE 63

### 3-(1H-Imidazol-2-ylmethyl)-1-[4-[4-[(3S)-3-methylmorpholin-4-yl]-6-(1-pyridin-4-ylsulfonylcyclobutyl)pyrimidin-2-yl]phenyl]thiourea

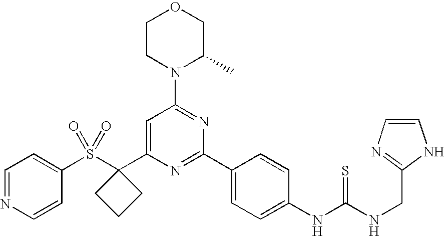

1,1′-Thiocarbonyldiimidazole (49.8 mg, 0.28 mmol) was added to a stirred solution of 4-[4-[(3S)-3-methylmorpholin-4-yl]-6-(1-pyridin-4-ylsulfonylcyclobutyl)pyrimidin-2-yl]aniline (100 mg, 0.21 mmol) in THF (1.0 mL) and DCM (2.0 mL) at RT. The resulting solution was stirred for 2 hours. Triethylamine (0.030 mL, 0.21 mmol) and (1H-imidazol-2-yl)methanamine (20.86 mg, 0.21 mmol) were then added to the reaction mixture and stirred at RT for 1 hour. The reaction mixture was evaporated to dryness and redissolved in acetonitrile (2.0 mL), filtered and then purified by preparative HPLC, eluting with decreasingly polar mixtures of water (containing 1% ammonia) and acetonitrile, to afford desired material as a white solid (85 mg).

NMR Spectrum: ¹H NMR (399.9 MHz, DMSO-d₆) δ 1.21-1.23 (3H, d), 1.94 (1H, m), 2.15 (1H, m), 2.82 (2H, m), 3.14 (2H, m), 3.51 (1H, t), 3.63 (1H, d), 3.68 (1H, d), 3.77 (1H, d), 3.97 (1H, dd), 4.17 (1H, d), 4.55 (1H, s), 4.73 (2H, s), 6.57 (1H, s), 6.89 (1H, s), 7.08 (1H, s), 7.48 (2H, d), 7.56 (2H, d), 7.79 (2H, d), 8.20 (1H, s), 8.73 (2H, d), 10.00 (1H, s), 11.94 (1H, s)

LCMS Spectrum: m/z (ESI+) (M+H)+=605.51; HPLC tR=2.03 min.

The preparation of 4-[4-[(3S)-3-methylmorpholin-4-yl]-6-(1-pyridin-4-ylsulfonylcyclobutyl)pyrimidin-2-yl]aniline is described below:

### 4-[4-[(3S)-3-Methylmorpholin-4-yl]-6-(1-pyridin-4-ylsulfonylcyclobutyl)pyrimidin-2-yl]aniline

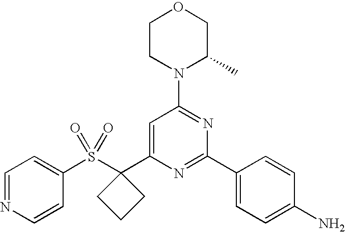

Bis(triphenylphosphine)palladium(II) chloride (79 mg, 0.11 mmol) was added to 2-chloro-4-[(3S)-3-methylmorpholin-4-yl]-6-(1-pyridin-4-ylsulfonylcyclobutyl)pyrimidine (920 mg, 2.25 mmol), 4-(4,4,5,5-tetramethyl-1,3,2-dioxaborolan-2-yl)aniline (641 mg, 2.92 mmol) and 2M aqueous sodium carbonate solution (5 mL, 10.0 mmol) in DMF (10 ML), DME (40 mL), ethanol (10 mL) and water (10 mL) at RT under a nitrogen atmosphere. The resulting mixture was stirred at 80° C. for 2 hours. The reaction mixture was then diluted with ethyl acetate (400 mL) and washed sequentially with water (200 mL) and then brine (250 mL). The organic layer was dried over MgSO₄, filtered and evaporated to afford crude product. The crude product was purified by flash silica chromatography, with an elution gradient of 0% to 50% ethyl acetate in DCM, to afford the desired material as a creamy white solid (1.01 g).

NMR Spectrum: ¹H NMR (399.9 MHz, DMSO-d₆) δ 1.19-1.21 (3H, m), 1.88-1.95 (1H, m), 2.10-2.15 (1H, m), 2.76-2.83 (2H, m), 3.05-3.18 (3H, m), 3.45-3.52 (1H, m), 3.62-3.65 (1H, m), 3.75 (1H, d), 3.94-3.98 (1H, m), 4.13 (1H, d), 4.47 (1H, d), 5.50 (2H, d), 6.45-6.48 (2H, m), 6.52 (1H, s), 7.44-7.46 (2H, m), 7.52-7.66 (2H, d), 8.70-8.72 (2H, m)

LCMS Spectrum: m/z (ESI+) (M+H)+=466.20; HPLC tR=2.06 min

### 2-Chloro-4-[(3S)-3-methylmorpholin-4-yl]-6-(1-pyridin-4-ylsulfonylcyclobutyl)pyrimidine

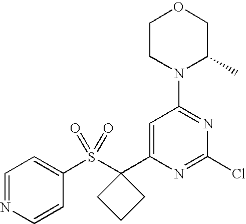

Sodium hydroxide (50% w/w solution) (24.39 g, 609.89 mmol) was added to a mixture of 2-chloro-4-[(3S)-3-methylmorpholin-4-yl]-6-(pyridin-4-ylsulfonylmethyl)pyrimidine (4.09 g, 11.09 mmol), 1,3-dibromopropane (3.38 mL, 33.27 mmol) and tetrabutylammonium bromide (0.357 g, 1.11 mmol) in toluene (200 mL) at RT under air. The resulting mixture was warmed to 45° C. for 3 hours. Water (100 mL) was added to the solution and the toluene layer was washed with further water, brine and then dried over MgSO₄. The mixture was filtered and the filtrate was evaporated to dryness. The crude product was purified by flash silica chromatography, eluting with a gradient of 30 to 50% ethyl acetate in DCM, to afford desired material as a solid (936 mg).

NMR Spectrum: ¹H NMR (399.9 MHz, DMSO-d₆) δ 1.17-1.19 (3H, m), 1.89-1.94 (1H, m), 2.07-2.13 (1H, m), 2.68-2.75 (2H, m), 2.96-3.06 (2H, m), 3.11-3.19 (1H, m), 3.39-3.46 (1H, m), 3.56-3.59 (1H, m), 3.71 (1H, d), 3.91-3.94 (2H, m), 4.34 (1H, s), 6.65 (1H, s), 7.47-7.49 (2H, m), 8.83-8.85 (2H, m)

LCMS Spectrum: m/z (ESI+) (M+H)+=409; HPLC tR=2.04 min.

The preparation of 2-chloro-4-[(3S)-3-methylmorpholin-4-yl]-6-(pyridin-4-ylsulfonylmethyl)pyrimidine was described earlier.

### EXAMPLE 64

### 3-Cyclopropyl-1-[4-[4-[1-[4-(difluoromethoxy)phenyl]sulfonylcyclopropyl]-6-[(3 S)-3-methylmorpholin-4-yl]pyrimidin-2-yl]phenyl]urea

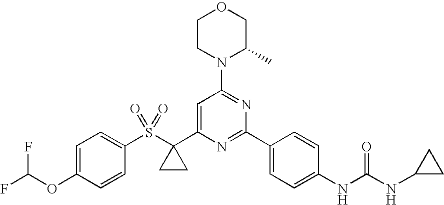

Cyclopropylamine (0.136 mL, 0.80 mmol) was added to a solution of phenyl N-[4-[4-[1-[4-(difluoromethoxy)phenyl]sulfonylcyclopropyl]-6-[(3S)-3-methylmorpholin-4-yl]pyrimidin-2-yl]phenyl]carbamate (100 mg, 0.16 mmol) and triethylamine (0.066 mL, 0.48 mmol) in DMA (1 mL). The reaction was stirred at RT for 18 hours. The crude product was purified by preparative HPLC, eluting with decreasingly polar mixtures of water (containing 1% ammonia) and acetonitrile to afford the desired product as a solid (66 mg).

NMR Spectrum: ¹H NMR (400.13 MHz, DMSO-d₆) δ 0.41 (2H, m), 0.64 (2H, m), 1.18 (3H, d), 1.61 (2H, m), 1.87 (2H, m), 2.55 (1H, dd), 3.14 (1H, m), 3.46 (1H, td), 3.61 (1H, dd), 3.74 (1H, d), 3.95 (1H, dd), 4.14 (1H, s), 4.40 (1H, s), 6.40 (1H, d), 6.63 (1H, s), 7.37-7.58 (5H, m), 7.84 (4H, m), 8.50 (1H, s)

LCMS Spectrum: m/z (ESI+)(M+H)+=600; HPLC tR=2.36 min.

The compounds below were prepared in an analogous fashion using the appropriate carbamate and the appropriate amine.

| | | | LCMS | Retention |
|---|---|---|---|---|
| Example | Structure | NAME | MH+ | time (min) |
| 64a* | 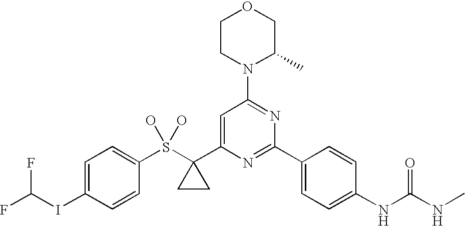 | 1-[4-[4-[1-[4-(difluoromethoxy)phenyl]sulfonyl-cyclopropyl]-6-[(3S)-3-methylmorpholin-4-yl]pyrimidin-2-yl]phenyl]-3-methylurea | 574 | 2.21 |
| 64b* | 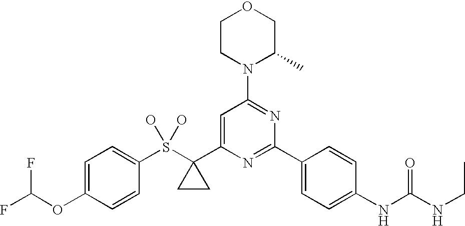 | 3-[4-[4-[1-[4-(difluoromethoxy)phenyl]sulfonyl-cyclopropyl]-6-[(3S)-3-methylmorpholin-4-yl]pyrimidin-2-yl]phenyl]-1-ethylurea | 588 | 2.35 |
| 64c* | 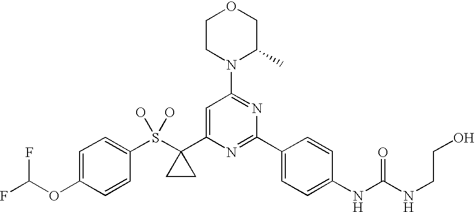 | 1-[4-[4-[1-[4-(difluoromethoxy)phenyl]sulfonyl-cyclopropyl]-6-[(3S)-3-methylmorpholin-4-yl]pyrimidin-2-yl]phenyl]-3-(2-hydroxyethyl)urea | 604 | 2.05 |
| 64d** | 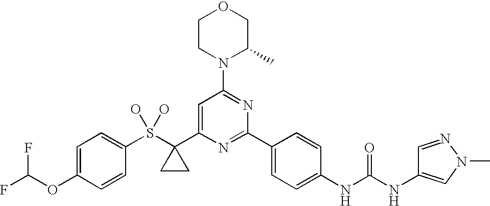 | 1-[4-[4-[1-[4-(difluoromethoxy)phenyl]sulfonyl-cyclopropyl]-6-[(3S)-3-methylmorpholin-4-yl]pyrimidin-2-yl]phenyl]-3-(1-methylpyrazol-4-yl)urea | 640 | 2.22 |
| 64e** | 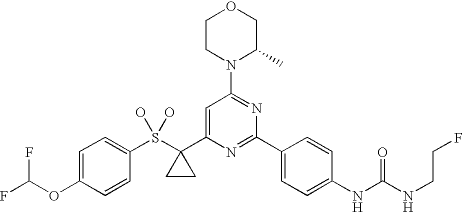 | 1-[4-[4-[1-[4-(difluoromethoxy)phenyl]sulfonyl-cyclopropyl]-6-[(3S)-3-methylmorpholin-4-yl]pyrimidin-2-yl]phenyl]-3-(2-fluoroethyl)urea | 606 | 2.33 |
| 64f** | 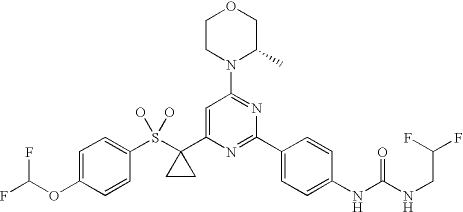 | 3-(2,2-difluoroethyl)-1-[4-[4-[1-[4-(difluoromethoxy)phenyl]sulfonyl-cyclopropyl]-6-[(3S)-3-methylmorpholin-4-yl]pyrimidin-2-yl]phenyl]urea | 624 | 2.45 |
| 64g* | 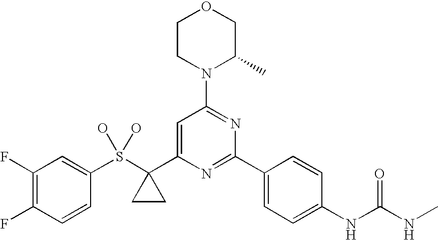 | 1-[4-[4-[1-(3,4-difluorophenyl)sulfonylcyclopro-pyl]-6-[(3S)-3-methylmorpholin-4-yl]pyrimidin-2-yl]phenyl]-3-methylurea | 544 | 2.21 |
| 64h* | 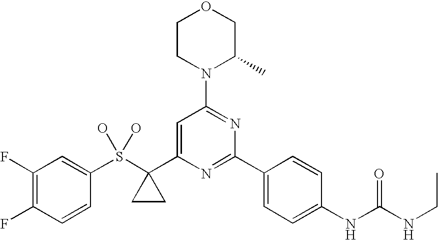 | 3-[4-[4-[1-(3,4-difluorophenyl)sulfonylcyclopro-pyl]-6-[(3S)-3-methylmorpholin-4-yl]pyrimidin-2-yl]phenyl]-1-ethylurea | 558 | 2.40 |
| 64i | 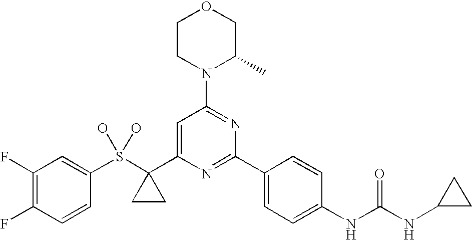 | 3-cyclopropyl-1-[4-[4-[1-(3,4-difluorophenyl)sulfonylcyclopro-pyl]-6-[(3S)-3-methylmorpholin-4-yl]pyrimidin-2-yl]phenyl]urea | 570 | 2.47 |
| 64j | 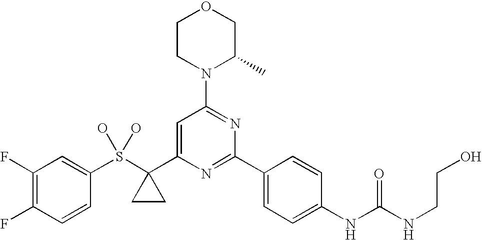 | 1-[4-[4-[1-(3,4-difluorophenyl)sulfonylcyclopro-pyl]-6-[(3S)-3-methylmorpholin-4-yl]pyrimidin-2-yl]phenyl]-3-(2-hydroxyethyl)urea | 574 | 2.06 |
| 64k** | 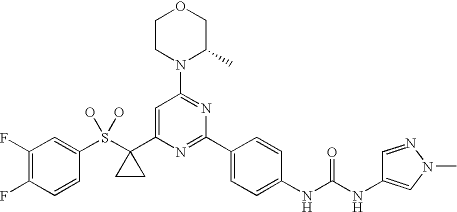 | 1-[4-[4-[1-(3,4-difluorophenyl)sulfonylcyclopro-pyl]-6-[(3S)-3-methylmorpholin-4-yl]pyrimidin-2-yl]phenyl]-3-(1-methylpyrazol-4-yl)urea | 610 | 2.22 |
| 641** | 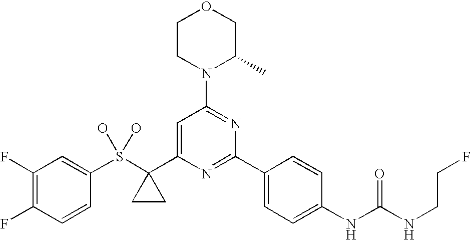 | 1-[4-[4-[1-(3,4-difluorophenyl)sulfonylcyclopro-pyl]-6-[(3S)-3-methylmorpholin-4-yl]pyrimidin-2-yl]phenyl]-3-(2-fluoroethyl)urea | 576 | 2.34 |
| 64m** | 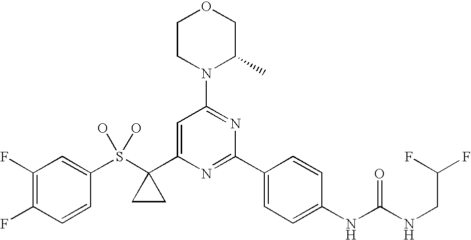 | 3-(2,2-difluoroethyl)-1-[4-[4-[1-(3,4-difluorophenyl)sulfonylcyclo-propyl]-6-[(3S)-3-methylmorpholin-4-yl]pyrimidin-2-yl]phenyl]urea | 594 | 2.45 |
| 64n*** | 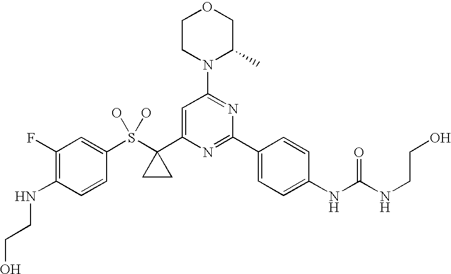 | 1-[4-[4-[1-[3-fluoro-4-(2-hydroxyethylamino)phenyl]sulfon-ylcyclopropyl]-6-[(3S)-3-methylmorpholin-4-yl]pyrimidin-2-yl]phenyl]-3-(2-hydroxyethyl)urea | 615 | 1.72 |
| 64o | 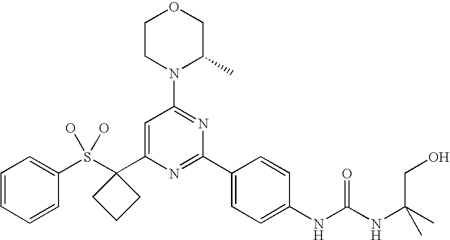 | 1-[4-[4-[1-(benzenesulfonyl)cyclobutyl]-6-[(3S)-3-methylmorpholin-4-yl]pyrimidin-2-yl]phenyl]-3-(1-hydroxy-2-methylpropan-2-yl)urea | 580 | 2.30 |
| 64p | 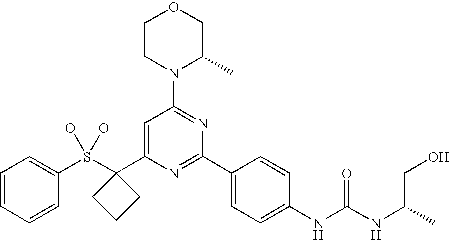 | 1-[4-[4-[1-(benzenesulfonyl)cyclobutyl]-6-[(3S)-3-methylmorpholin-4-yl]pyrimidin-2-yl]phenyl]-3-[(2S)-1-hydroxypropan-2-yl]urea | 566 | 2.11 |
| 64q | 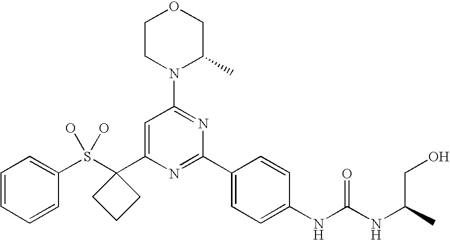 | 1-[4-[4-[1-(benzenesulfonyl)cyclobutyl]-6-[(3S)-3-methylmorpholin-4-yl]pyrimidin-2-yl]phenyl]-3-[(2R)-1-hydroxypropan-2-yl]urea | 566 | 2.11 |
| 64r | 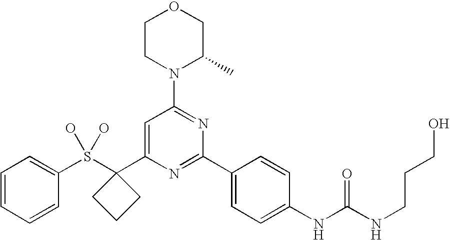 | 1-[4-[4-[1-(benzenesulfonyl)cyclobutyl]-6-[(3S)-3-methylmorpholin-4-yl]pyrimidin-2-yl]phenyl]-3-(3-hydroxypropyl)urea | 566 | 2.05 |
| 64s** | 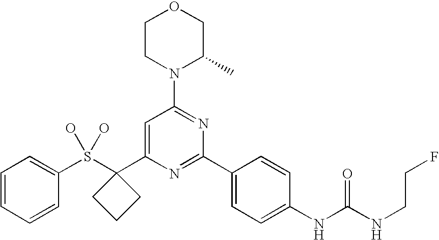 | 1-[4-[4-[1-(benzenesulfonyl)cyclobutyl]-6-[(3S)-3-methylmorpholin-4-yl]pyrimidin-2-yl]phenyl]-3-(2-fluoroethyl)urea | 554 | 2.32 |
| 64t** | 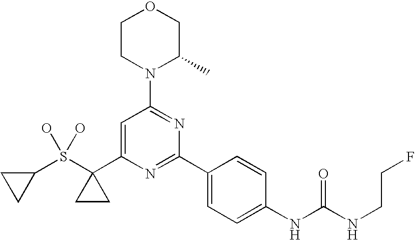 | 1-[4-[4-(1-cyclopropylsulfonylcyclopropyl)-6-[(3S)-3-methylmorpholin-4-yl]pyrimidin-2-yl]phenyl]-3-(2-fluoroethyl)urea | 504 | 1.95 |
| 64u** | 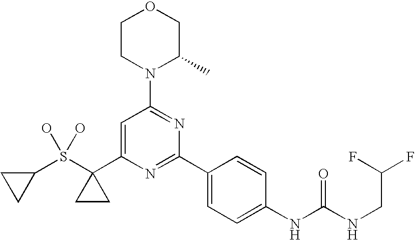 | 1-[4-[4-(1-cyclopropylsulfonylcyclopropyl)-6-[(3S)-3-methylmorpholin-4-yl]pyrimidin-2-yl]phenyl]-3-(2,2-difluoroethyl)urea | 522 | 2.10 |
| 64v | 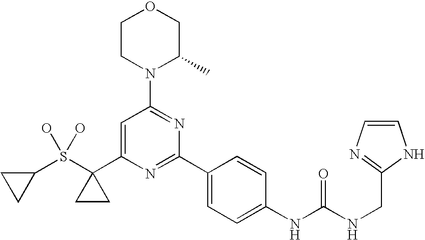 | 1-[4-[4-(1-cyclopropylsulfonylcyclopropyl)-6-[(3S)-3-methylmorpholin-4-yl]pyrimidin-2-yl]phenyl]-3-(1H-imidazol-2-ylmethyl)urea | 538 | 1.77 |
| 64w | 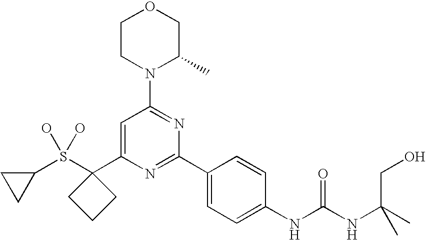 | 1-[4-[4-(1-cyclopropylsulfonylcyclobutyl)-6-[(3S)-3-methylmorpholin-4-yl]pyrimidin-2-yl]phenyl]-3-(1-hydroxy-2-methylpropan-2-yl)urea | 544 | 2.11 |
| 64x | 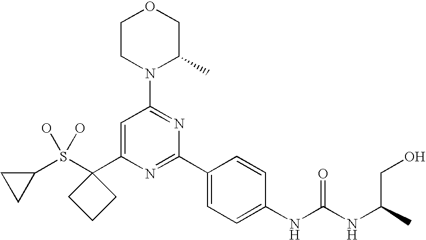 | 1-[4-[4-(1-cyclopropylsulfonylcyclobutyl)-6-[(3S)-3-methylmorpholin-4-yl]pyrimidin-2-yl]phenyl]-3-[(2S)-1-hydroxypropan-2-yl]urea | 530 | 1.91 |
| 64y | 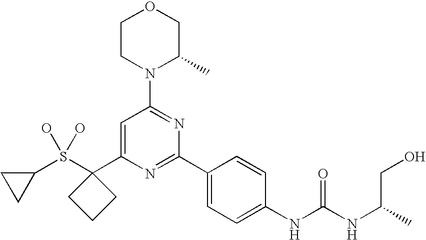 | 1-[4-[4-(1-cyclopropylsulfonylcyclobutyl)-6-[(3S)-3-methylmorpholin-4-yl]pyrimidin-2-yl]phenyl]-3-[(2R)-1-hydroxypropan-2-yl]urea | 530 | 1.91 |
| 64z | 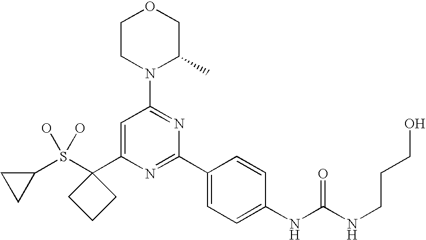 | 1-[4-[4-(1-cyclopropylsulfonylcyclobutyl)-6-[(3S)-3-methylmorpholin-4-yl]pyrimidin-2-yl]phenyl]-3-(3-hydroxypropyl)urea | 530 | 1.86 |
| 64aa** | 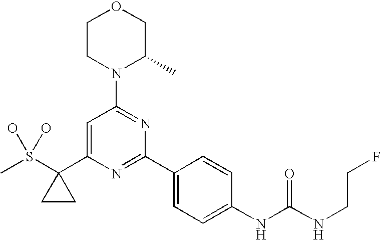 | 3-(2-fluoroethyl)-1-[4-[4-[(3S)-3-methylmorpholin-4-yl]-6-(1-methylsulfonylcyclopropyl)pyrimi-din-2-yl]phenyl]urea | 478 | 1.84 |
| 64ab** | 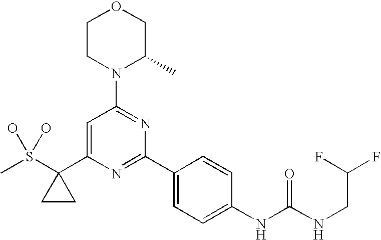 | 3-(2,2-difluoroethyl)-1-[4-[4-[(3S)-methylsulfonylcyclopropyl)pyrimi-din-2-yl]phenyl]urea | 496 | 2.03 |
| 64ac | 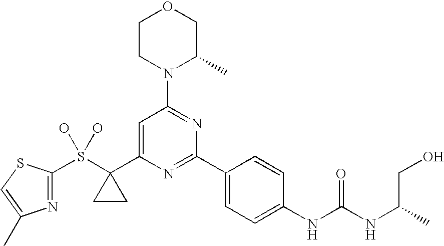 | 3-[(2S)-1-hydroxypropan-2-yl]-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 573 | 1.92 |
| 64ad | 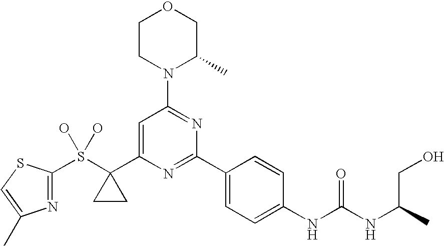 | 3-[(2R)-1-hydroxypropan-2-yl]-1-[4-[4-[(3S)-3-methylmorpholin-4-yl]-6-[1-[(4-methyl-1,3-thiazol-2-yl)sulfonyl]cyclopropyl]pyrimidin-1-yl]phenyl]urea | 561 | 2.14 |
| 64ae** | 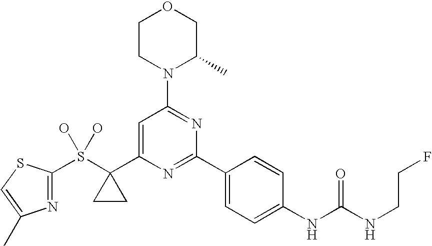 | 3-(2-fluoroethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea | 561 | 2.14 |
| 64af** | 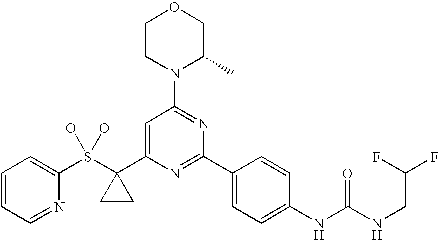 | 3-(2,2-difluoroethyl)-1-[4-[4-[(3S)-3-methylmorpholin-4-yl]-6-(1-pyridin-2-ylsulfonylcyclopropyl)pyrimidin-2-yl]phenyl]urea | 559 | 2.07 |
| 64ag | 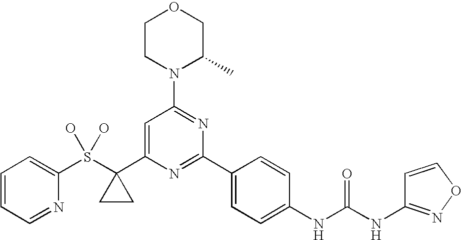 | 1-[4-[4-[(3S)-3-methylmorpholin-4-yl]-6-(1-pyridin-2-ylsulfonylcyclopropyl)pyrimidin-2-yl]phenyl]-3-(1,2-oxazol-3-yl)urea | 562 | 2.12 |
| 64ah** | 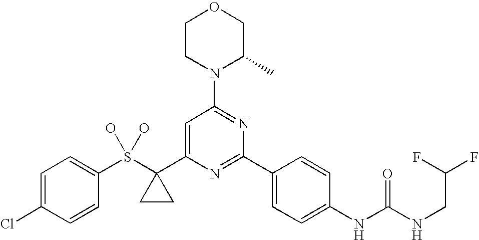 | 1-[4-[4-[1-(4-chlorophenyl)sulfonylcyclopropyl]-6-[(3S)-3-methylmorpholin-4-yl]pyrimidin-2-yl]phenyl]-3-(2,2-difluoroethyl)urea | 592 | 2.48 |
| 64ai | 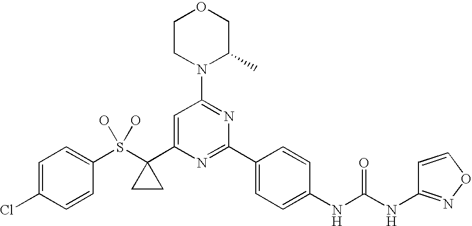 | 1-[4-[4-[1-(4-chlorophenyl)sulfonylcyclopropyl]-6-[(3S)-3-methylmorpholin-4-yl]pyrimidin-2-yl]phenyl]-3-(1,2-oxazol-3-yl)urea | 595 | 2.51 |
| 64aj** | 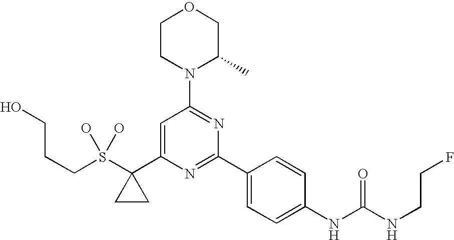 | 3-(2-fluoroethyl)-1-[4-[4-[1-(3-hydroxypropylsulfonyl)cyclopro-pyl]-6-[(3S)-3-methylmorpholin-4-yl]pyrimidin-2-yl]phenyl]urea | 522 | 1.72 |
| 64ak | 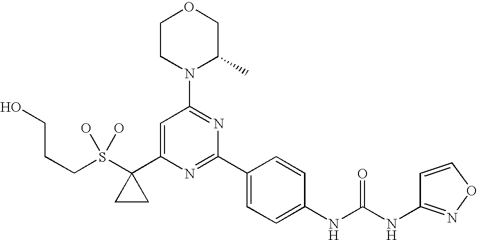 | 1-[4-[4-[1-(3-hydroxypropylsulfonyl)cyclopro-pyl]-6-[(3S)-3-methylmorpholin-4-yl]pyrimidin-2-yl]phenyl]-3-(1,2-oxazol-3-yl)urea | 543 | 1.77 |
| 64al* | 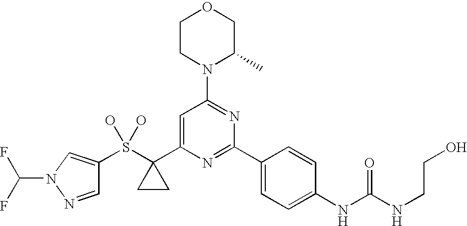 | 1-[4-[4-[1-[1-(difluoromethyl)pyrazol-4-yl]sulfonylcyclopropyl]-6-[(3S)-3-methylmorpholin-4-yl]pyrimidin-2-yl]phenyl]-3-(2-hydroxyethyl)urea | 578 | 2.00 |

*1.2 Equivalents of amine used
**Reaction heated at 50° C. for 3 hours
***By product formed and isolated when preparing Example 64j

### EXAMPLE 64a

¹H NMR (400.132 MHz, DMSO-d₆) δ 1.18 (3H, d), 1.60 (2H, m), 1.89 (2H, m), 2.66 (3H, d), 3.14 (1H, td), 3.46 (1H, td), 3.62 (1H, dd), 3.74 (1H, d), 3.94 (1H, dd), 4.15 (1H, d), 4.40 (1H, s), 6.04 (1H, q), 6.63 (1H, s), 7.36-7.61 (5H, m), 7.84 (4H, m), 8.70 (1H, s)

### EXAMPLE 64b

¹H NMR (400.132 MHz, DMSO-d₆) δ 1.06 (3H, t), 1.18 (3H, d), 1.61 (2H, m), 1.88 (2H, m), 3.13 (3H, m), 3.46 (1H, td), 3.61 (1H, dd), 3.74 (1H, d), 3.95 (1H, dd), 4.15 (1H, d), 4.40 (1H, s), 6.13 (1H, t), 6.63 (1H, s), 7.37-7.59 (5H, m), 7.84 (4H, m), 8.62 (1H, s)

### EXAMPLE 64c

¹H NMR (400.132 MHz, DMSO-d₆) δ 1.18 (3H, d), 1.60 (2H, m), 1.87 (2H, m), 3.16-3.18 (3H, m), 3.46 (3H, m), 3.61 (1H, dd), 3.75 (1H, d), 3.95 (1H, dd), 4.15 (1H, d), 4.40 (1H, s), 4.72 (1H, t), 6.24 (1H, t), 6.63 (1H, s), 7.37-7.60 (5H, m), 7.84 (4H, m), 8.76 (1H, s)

### EXAMPLE 64d

¹H NMR (400.132 MHz, DMSO-d₆) δ 1.19 (3H, d), 1.61 (2H, m), 1.89 (2H, m), 3.17 (1H, td), 3.46 (1H, td), 3.61 (1H, dd), 3.75 (1H, d), 3.79 (3H, s), 3.96 (1H, dd), 4.15 (1H, d), 4.40 (1H, s), 6.65 (1H, s), 7.36-7.60 (6H, m), 7.76 (1H, s), 7.84-7.86 (4H, dd), 8.35 (1H, s), 8.80 (1H, s)

### EXAMPLE 64e

¹H NMR (400.132 MHz, DMSO-d₆) δ 1.18 (3H, d), 1.61 (2H, m), 1.89 (2H, m), 3.16 (1H, td), 3.38 (1H, m), 3.45 (2H, m), 3.61 (1H, dd), 3.74 (1H, d), 3.95 (1H, dd), 4.15 (1H, d), 4.41 (2H, t), 4.53 (1H, t), 6.41 (1H, t), 6.64 (1H, s), 7.37-7.60 (5H, m), 7.84 (4H, m), 8.77 (1H, s)

### EXAMPLE 64f

¹H NMR (400.132 MHz, DMSO-d₆) δ 1.18 (3H, d), 1.60 (2H, m), 1.89 (2H, m), 3.16 (1H, td), 3.48-3.54 (3H, m), 3.61 (1H, dd), 3.74 (1H, d), 3.96 (1H, dd), 4.14 (1H, d), 4.40 (1H, s), 5.90-6.40 (1H, m), 6.50 (1H, t), 6.64 (1H, s), 7.38-7.60 (5H, m), 7.85 (4H, m), 8.88 (1H, s)

### EXAMPLE 64g

¹H NMR (400.132 MHz, DMSO-d₆) δ 1.19 (3H, m), 1.61 (2H, dd), 1.93 (2H, dd), 2.67 (3H, t), 3.17 (1H, m), 3.46 (1H, td), 3.62 (1H, dd), 3.75 (1H, m), 3.92 (1H, dd), 4.20 (1H, d), 4.45 (1H br s), 6.04 (1H, q), 6.67 (1H, s), 7.40 (2H, m), 7.67 (2H, m), 7.79 (2H, m), 7.91 (1H, m), 8.73 (1H, s)

### EXAMPLE 64h

¹H NMR (400.132 MHz, DMSO-d₆) δ 1.07 (3H, t), 1.19 (3H, d), 1.61 (2H, dd), 1.93 (2H, dd), 3.12 (2H, m), 3.16 (1H, m), 3.45 (1H, td), 3.62 (1H, dd), 3.75 (1H, d), 3.95 (1H, dd), 4.15 (1H, d), 4.45 (1H, br s), 6.13 (1H, t), 6.67 (1H, s), 7.39 (2H, m), 7.65 (1H, d), 7.67 (1H, t), 7.79 (2H, m), 7.91 (1H, m), 8.65 (1H, s)

### EXAMPLE 64i

¹H NMR (400.132 MHz, DMSO-d₆) δ 0.42 (2H, dt), 0.65 (2H, td), 1.19 (3H, d), 1.61 (2H, dd), 1.93 (2H, dd), 2.56 (1H, m), 3.18 (1H, td), 3.46 (1H, td), 3.61 (1H, dd), 3.75 (1H, d), 3.96 (1H, dd), 4.15 (1H, d), 4.47 (1H, br s), 6.30 (1H, s), 6.67 (1H, s), 7.40 (2H, m), 7.66 (2H, m), 7.80 (2H, m), 7.90 (1H, td), 8.53 (1H, s)

### EXAMPLE 64j

¹H NMR (400.132 MHz, DMSO-d₆) δ 1.19 (3H, d), 1.61 (2H, dd), 1.93 (2H, m), 3.17 (3H, q), 3.45 (3H, m), 3.61 (1H, dd), 3.73 (1H, d), 3.91 (1H, dd), 4.15 (1H, d), 4.50 (1H, br s), 4.72 (1H, t), 6.22 (1H, s), 6.67 (1H, s), 7.38 (2H, m), 7.66 (1H, t), 7.67 (1H, s), 7.79 (2H, m), 7.90 (1H, m), 8.79 (1H, s)

### EXAMPLE 64k

¹H NMR (400.132 MHz, DMSO-d₆) δ 1.20 (3H, d), 1.62 (2H, dd), 1.94 (2H, dd), 3.17 (1H, td), 3.47 (1H, td), 3.62 (1H, dd), 3.74 (1H, d), 3.79 (3H, s), 3.96 (1H, dd), 4.17 (1H, d), 4.47 (1H, br s), 6.68 (1H, s), 7.38 (1H, d), 7.45 (2H, m), 7.67 (2H, m), 7.77 (1H, d), 7.83 (2H, d), 7.91 (1H, t), 8.35 (1H, s), 8.84 (1H, s)

### EXAMPLE 64l

¹H NMR (400.132 MHz, DMSO-d₆) δ 1.20 (3H, m), 1.61 (2H, dd), 1.93 (2H, dd), 3.17 (1H, td), 3.39 (1H, t), 3.45 (2H, q), 3.61 (1H, dd), 3.75 (1H, d), 3.96 (1H, dd), 4.15 (1H, d), 4.42 (1H, t), 4.50 (1H, br s), 4.53 (1H, t), 6.40 (1H, t), 6.67 (1H, s), 7.40 (2H, m), 7.66 (1H, d), 7.67 (1H, t), 7.80 (2H, d), 7.90 (1H, m), 8.80 (1H, s)

### EXAMPLE 64m

¹H NMR (400.132 MHz, DMSO-d₆) δ 1.20 (3H, d), 1.61 (2H, dd), 1.94 (2H, dd), 3.17 (1H, td), 3.45 (1H, td), 3.54 (2H, m), 3.61 (1H, dd), 3.75 (1H, d), 3.96 (1H, dd), 4.17 (1H, d), 4.47 (1H, br s), 5.90-6.20 (1H, tt), 6.50 (1H, t), 6.68 (1H, s), 7.41 (2H, m), 7.67 (2H, m), 7.82 (2H, m), 7.90 (1H, m), 8.92 (1H, s)

### EXAMPLE 64n

¹H NMR (400.132 MHz, DMSO-d₆) δ 1.18 (3H, d), 1.56 (2H, m), 1.81 (2H, m), 3.15 (1H, m), 3.17 (2H, m), 3.46 (3H, q), 3.57 (2H, q), 3.62 (1H, m), 3.75 (1H, d), 3.96 (1H, dd), 4.06 (1H, m), 4.37 (1H, br s), 4.73 (2H, q), 6.25 (1H, t), 6.36 (1H, m), 6.58 (1H, s), 6.82 (1H, t), 7.33 (1H, m), 7.41 (3H, m), 7.96 (2H, m), 8.76 (1H, s)

### EXAMPLE 64o

¹H NMR (400.132 MHz, DMSO-d₆) δ 1.19 (3H, d), 1.22 (6H, s), 1.87 (1H, 2.08 (1H, m), 2.78 (2H, m), 3.04-3.20 (3H, m), 3.38 (2H, d), 3.48 (1H, td), 3.63 (1H, dd), 3.75 (1H, d), 3.96 (1H, dd), 4.08 (1H, d), 4.44 (1H, br s), 4.95 (1H, t), 5.97 (1H, s), 6.46 (1H, s), 7.32 (2H, m), 7.47 (4H, m), 7.60 (1H, m), 7.78 (2H, d), 8.67 (1H, s)

### EXAMPLE 64p

¹H NMR (400.132 MHz, DMSO-d₆) δ 1.08 (3H, d), 1.20 (3H, d), 1.87 (1H, m), 2.08 (1H, m), 2.79 (2H, m), 3.04-3.20 (3H, m), 3.36 (2H, m), 3.48 (1H, td), 3.63 (1H, dd), 3.71 (2H, m), 3.96 (1H, dd), 4.07 (1H, d), 4.45 (1H, br s), 4.78 (1H, t), 6.07 (1H, m), 6.46 (1H, s), 7.35 (2H, m), 7.47 (4H, m), 7.60 (1H, m), 7.79 (2H, d), 8.65 (1H, s)

### EXAMPLE 64q

¹H NMR (400.132 MHz, DMSO-d₆) δ 1.08 (3H, d), 1.20 (3H, d), 1.87 (1H, m), 2.08 (1H, m), 2.79 (2H, m), 3.04-3.20 (3H, m), 3.36 (2H, m), 3.48 (1H, td), 3.63 (1H, dd), 3.71 (1H, m), 3.73 (1H, m), 3.96 (1H, dd), 4.07 (1H, d), 4.45 (1H, br s), 4.78 (1H, t), 6.07 (1H, d), 6.46 (1H, s), 7.35 (2H, d), 7.47 (4H, m), 7.59 (1H, td), 7.79 (2H, m), 8.65 (1H, s)

### EXAMPLE 64r

¹H NMR (400.132 MHz, DMSO-d₆) δ 1.20 (3H, d), 1.59 (2H, m), 1.87 (1H, m), 2.08 (1H, m), 2.79 (2H, m), 3.04 (2H, m), 3.12 (1H, m), 3.25 (2H, d), 3.46 (3H, m), 3.63 (1H, dd), 3.75 (1H, d), 3.96 (1H, dd), 4.07 (1H, m), 4.47 (2H, m), 6.18 (1H, t), 6.46 (1H, s), 7.36 (2H, m), 7.48 (4H, m), 7.59 (1H, m), 7.79 (2H, d), 8.65 (1H, s)

### EXAMPLE 64s

¹H NMR (400.132 MHz, DMSO-d₆) δ 1.20 (3H, d), 1.87 (1H, m), 2.12 (1H, m), 2.75 (2H, m), 3.00-3.20 (3H, m), 3.40 (1H, t), 3.45 (2H, m), 3.61 (1H, dd), 3.74 (1H, d), 3.92 (1H, dd), 4.15 (1H, d), 4.41 (2H, m), 4.53 (1H, t), 6.41 (1H, t), 6.47 (1H, s), 7.37 (2H, m), 7.48 (4H, m), 7.60 (1H, t), 7.80 (2H, m), 8.75 (1H, s)

### EXAMPLE 64t

¹H NMR (400.132 MHz, DMSO-d₆) δ 0.94 (2H, m), 1.03 (2H, m), 1.24 (3H, d), 1.58 (2H, m), 1.65 (2H, m), 3.00 (1H, m), 3.15 (1H, td), 3.36 (1H, m), 3.46 (2H, m), 3.61 (1H, dd), 3.77 (1H, d), 3.94 (1H, dd), 4.20 (1H, d), 4.42 (1H, t), 4.53 (2H, t), 6.43 (1H, t), 6.85 (1H, s), 7.50 (2H, m), 8.22 (2H, m), 8.80 (1H, s)

### EXAMPLE 64u

¹H NMR (400.132 MHz, DMSO-d₆) δ 0.94 (2H, m), 1.03 (2H, m), 1.24 (3H, d), 1.59 (2H, m), 1.65 (2H, m), 3.00 (1H, m), 3.21 (1H, td), 3.54 (3H, m), 3.64 (1H, dd), 3.77 (1H, d), 3.98 (1H, dd), 4.20 (1H, d), 4.54 (1H, br s), 5.90-6.20 (1H, tt), 6.53 (1H, t), 6.85 (1H, s), 7.51 (2H, m), 8.23 (2H, m), 8.91 (1H, s)

### EXAMPLE 64v

¹H NMR (400.132 MHz, DMSO-d₆) δ 0.94 (2H, m), 1.03 (2H, m), 1.24 (3H, d), 1.59 (2H, m), 1.65 (2H, m), 3.00 (1H, m), 3.19 (1H, td), 3.49 (1H, td), 3.64 (1H, dd), 3.77 (1H, d), 3.96 (1H, dd), 4.20 (1H, d), 4.32 (2H, d), 4.54 (1H, br s), 6.62 (1H, t), 6.85 (1H, s), 6.95 (1H, br s), 7.51 (2H, m), 8.22 (2H, m), 8.92 (1H, s)

### EXAMPLE 64w

¹H NMR (400.132 MHz, DMSO-d₆) δ 0.76 (2H, m), 0.85 (2H, m), 1.24 (9H, m), 1.90 (1H, m), 2.10 (1H, m), 2.45 (1H, m), 2.80-3.30 (4H, m), 3.20 (1H, td), 3.39 (2H, d), 3.50 (1H, td), 3.65 (1H, dd), 3.75 (1H, d), 3.95 (1H, dd), 4.20 (1H, d), 4.50 (1H, br s), 4.95 (1H, t), 6.00 (1H, s), 6.70 (1H, s), 7.45 (2H, m), 8.22 (2H, m), 8.72 (1H, s)

### EXAMPLE 64x

¹H NMR (400.132 MHz, DMSO-d₆) δ 0.70 (2H, m), 0.85 (2H, m), 1.10 (3H, d), 1.25 (3H, d), 1.90 (1H, m), 2.10 (1H, m), 2.45 (1H, m), 2.80-3.00 (4H, m), 3.15 (1H, td), 3.35 (2H, m), 3.50 (1H, td), 3.60-3.80 (3H, m), 3.95 (1H, dd), 4.20 (1H, d), 4.55 (1H, br s), 4.75 (1H, t), 6.10 (1H, d), 6.70 (1H, s), 7.45 (2H, m), 8.20 (2H, m), 8.70 (1H, s)

### EXAMPLE 64y

¹H NMR (400.132 MHz, DMSO-d₆) δ 0.70 (2H, m), 0.84 (2H, m), 1.08 (3H, d), 1.23 (3H, d), 1.89 (1H, m), 2.10 (1H, m), 2.46 (1H, m), 2.84-3.00 (4H, m), 3.15 (1H, td), 3.36 (2H, m), 3.51 (1H, td), 3.67 (1H, td), 3.77 (2H, m), 3.95 (1H, dd), 4.20 (1H, d), 4.55 (1H, br s), 4.78 (1H, t), 6.09 (1H, d), 6.70 (1H, s), 7.47 (2H, m), 8.23 (2H, m), 8.70 (1H, s)

### EXAMPLE 64z

¹H NMR (400.132 MHz, DMSO-d₆) δ 0.70 (2H, m), 0.84 (2H, m), 1.20 (3H, d), 1.57 (2H, m), 1.89 (1H, m), 2.10 (1H, m), 2.46 (1H, m), 2.80-3.00 (4H, m), 3.15 (4H, m), 3.45 (2H, m), 3.55 (1H, td), 3.65 (1H, dd), 3.77 (1H, d), 3.95 (1H, dd), 4.20 (1H, d), 4.45 (1H, t), 4.55 (1H, br s), 6.20 (1H, t), 6.70 (1H, s), 7.47 (2H, m), 8.25 (2H, m), 8.70 (1H, s)

### EXAMPLE 64aa

¹H NMR (400.132 MHz, DMSO-d₆) δ 1.23 (3H, m), 1.58 (2H, m), 1.67 (2H, m), 2.47 (3H, s), 3.20 (1H, td), 3.39 (1H, q), 3.46 (2H, m), 3.63 (1H, dd), 3.76 (1H, d), 3.97 (1H, dd), 4.21 (1H, d), 4.42 (1H, t), 4.54 (1H, br s), 4.55 (1H, t), 6.43 (1H, t), 6.77 (1H, s), 7.51 (2H, m), 8.21 (2H, m), 8.82 (1H, s)

### EXAMPLE 64ab

¹H NMR (400.132 MHz, DMSO-d₆) δ 1.23 (3H, d), 1.56 (2H, m), 1.67 (2H, m), 2.46 (3H, s), 3.15 (1H, td), 3.47-3.63 (4H, m), 3.76 (1H, d), 3.97 (1H, dd), 4.21 (1H, d), 4.57 (1H, br s), 5.90-6.20 (1H, m), 6.53 (1H, t), 6.78 (1H, s), 7.52 (2H, m), 8.22 (2H, m), 8.93 (1H, s

### EXAMPLE 64ac

¹H NMR (400.132 MHz, DMSO-d₆) δ 1.08 (3H, d), 1.21 (3H, d), 1.77 (2H, m), 1.94 (2H, m), 2.48 (3H, d), 3.18 (1H, td), 3.36 (2H, m), 3.40 (1H, td), 3.63 (1H, dd), 3.71 (1H, m), 3.76 (1H, d), 3.96 (1H, dd), 4.15 (1H, d), 4.45 (1H, br s), 4.78 (1H, t), 6.07 (1H, m), 6.77 (1H, s), 7.39 (2H, m), 7.84-7.90 (3H, m), 8.69 (1H, s)

### EXAMPLE 64ad

¹H NMR (400.132 MHz, DMSO-d₆) δ 1.08 (3H, d), 1.21 (3H, d), 1.77 (2H, m), 1.94 (2H, m), 2.48 (3H, d), 3.18 (1H, td), 3.36 (2H, m), 3.40 (1H, td), 3.63 (1H, dd), 3.71 (1H, m), 3.76 (1H, d), 3.96 (1H, dd), 4.15 (1H, d), 4.45 (1H, br s), 4.78 (1H, t), 6.07 (1H, m), 6.77 (1H, s), 7.39 (2H, m), 7.84-7.90 (3H, m), 8.70 (1H, s)

### EXAMPLE 64ae

¹H NMR (400.132 MHz, DMSO-d₆) δ 1.21 (3H, d), 1.77 (2H, m), 1.96 (2H, m), 2.48 (3H, d), 3.18 (1H, td), 3.38 (1H, m), 3.45 (2H, m), 3.62 (1H, dd), 3.76 (1H, d), 3.96 (1H, dd), 4.15 (1H, d), 4.41 (1H, t), 4.45 (1H, br s), 4.53 (1H, t), 6.41 (1H, t), 6.78 (1H, s), 7.42 (2H, m), 7.84 (1H, s), 7.90 (2H, m), 8.79 (1H, s)

### EXAMPLE 64af

¹H NMR (400.132 MHz, DMSO-d₆) δ 1.16 (3H, d), 1.74 (2H, m), 1.97 (2H, m), 3.15 (1H, td), 3.40(1H, td), 3.50-3.60 (3H, m), 3.74 (1H, d), 3.95 (1H, dd), 4.13 (1H, d), 4.40 (1H, br s), 5.90-6.20 (1H, m), 6.50 (1H, t), 6.70 (1H, s), 7.35 (2H, m), 7.76 (3H, m), 7.95 (1H, m), 8.09 (1H, td), 8.75 (1H, d), 8.83 (1H, s)

### EXAMPLE 64ag

¹H NMR (400.132 MHz, DMSO-d₆) δ 1.16 (3H, d), 1.74 (2H, m), 2.00 (2H, m), 3.15 (1H, td), 3.52 (1H, td), 3.60 (1H, dd), 3.74 (1H, d), 3.95 (1H, dd), 4.13 (1H, d), 4.40 (1H, br s), 6.70 (1H, s), 6.87 (1H, s), 7.43 (2H, m), 7.76-7.81 (3H, m), 7.99 (1H, m), 8.09 (1H, td), 8.75 (1H, s), 8.83 (1H, m), 9.02 (1H, s), 9.60 (1H, s)

### EXAMPLE 64ah

¹H NMR (400.132 MHz, DMSO-d₆) δ 1.19 (3H, d), 1.61 (2H, dd), 1.90 (2H, dd), 3.17 (1H, m), 3.40-3.64 (4H, m), 3.75 (1H, d), 3.96 (1H, dd), 4.15 (1H, d), 4.45 (1H, br s), 5.90-6.20(1H, t), 6.49 (1H, t), 6.64 (1H, s), 7.41 (2H, d), 7.66 (2H, m), 7.78 (4H, m), 8.90 (1H, s)

### EXAMPLE 64ai

¹H NMR (400.132 MHz, DMSO-d₆) δ 1.19 (3H, m), 1.62 (2H, m), 1.91 (2H, m), 3.15 (1H, td), 3,45 (1H, td), 3.61 (1H, dd), 3.75 (1H, d), 3.95 (1H, dd), 4.15 (1H, d), 4.45 (1H, br s), 6.66 (1H, s), 6.87 (1H, s), 7.47 (2H, d), 7.67 (2H, d), 7.79 (2H, d), 7.84 (2H, m), 8.75 (1H, s), 9.10 (1H, s), 9.60 (1H, s)

### EXAMPLE 64aj

¹H NMR (400.132 MHz, DMSO-d₆) δ 1.22 (3H, d), 1.56 (2H, m), 1.64 (2H, m), 1.94 (2H, m), 3.15-3.25 (2H, m), 3.39 (1H, m), 3.40-3.52 (5H, m), 3.65 (1H, dd), 3.75 (1H, d), 3.95 (1H, dd), 4.20 (1H, br s, 4.42 (1H, t), 4.54 (2H, m), 4.69 (1H, t), 6.43 (1H, t), 6.77 (1H, s), 7.50 (2H, m), 8.22 (2H, m), 8.81 (1H, s)

### EXAMPLE 64ak

¹H NMR (400.132 MHz, DMSO-d₆) δ 1.24 (3H, d), 1.57 (2H, m), 1.64 (2H, m), 1.94 (2H, m), 3.25 (1H, m), 3.51 (1H, m), 3.53 (4H, m), 3.76 (1H, dd), 3.86 (1H, d), 4.10 (1H, dd), 4.30 (1H, d), 4.65 (1H, br s), 4.70 (1H, t), 6.80 (1H, s), 6.87 (1H, s), 7.57 (2H, m), 8.29 (2H, m), 8.76 (1H, s), 9.07 (1H, s), 9.62 (1H, s)

### EXAMPLE 64al

¹H NMR (400.132 MHz, DMSO-d₆) δ 1.20 (3H, d), 1.60 (2H, m), 1.85 (2H, m), 2.78 (1H, s), 2.95 (1H, s), 3.15 (2H, m), 3.42 (2H, m), 3.61 (1H, dd), 3.75 (1H, d), 3.96 (1H, dd), 4.15 (1H, d), 4.45 (1H, s), 4.72 (1H, t), 6.23 (1H, t), 6.70 (1H, s), 7.40 (2H, m), 7.70-8.00 (3H, m), 8.20 (1H, s), 8.80 (1H, s), 8.91 (1H, s)

The preparation of phenyl N-[4-[4-[1-[4-(difluoromethoxy)phenyl]sulfonylcyclopropyl]-6-[(3S)-3-methylmorpholin-4-yl]pyrimidin-2-yl]phenyl]carbamate is described below.

### Phenyl N-[4-[4-[1-[4-(difluoromethoxy)phenyl]sulfonylcyclopropyl]-6-[(3S)-3-methylmorpholin-4-yl]pyrimidin-2-yl]phenyl]carbamate

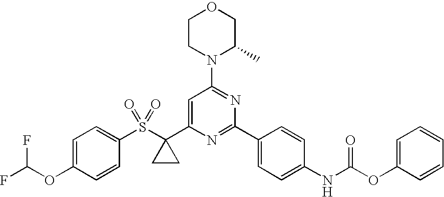

Phenyl chloroformate (0.170 mL, 1.36 mmol) was added dropwise to 4-[4-[1-[4-(difluoromethoxy)phenyl]sulfonylcyclopropyl]-6-[(3S)-3-methylmorpholin-4-yl]pyrimidin-2-yl]aniline (0.700 g, 1.36 mmol) and sodium hydrogen carbonate (0.114 g, 1.36 mmol) in dioxane and the resulting suspension stirred at RT for 6 hours. The reaction mixture was concentrated and diluted with DCM (50 mL), and washed sequentially with water (10 mL), water (10 mL), and saturated brine (10 mL). The organic layer was dried over MgSO₄, filtered and evaporated to afford crude product. The crude solid was triturated with a mixture of diethyl ether:isohexane (20:80) to give the desired material as a white solid (0.710 g).

NMR Spectrum: ¹H NMR (400.132 MHz, DMSO-d₆) δ 1.19 (3H, m), 1.62 (2H, dd), 1.90 (2H, dd), 3.16 (1H, td), 3.46 (1H, td), 3.60 (1H, dd), 3.75 (1H, d), 3.96 (1H, dd), 4.14 (1H, d), 4.42 (1H, s), 6.67 (1H, s), 7.24-7.27 (3H, m), 7.37 (3H, m), 7.45 (2H, m), 7.53 (2H, m), 7.84 (2H, m), 7.93 (2H, m), 10.40 (1H, s).

LCMS Spectrum: m/z (ES+)(M+H)+=637; HPLC tR=3.04 min.

### 4-[4-[1-[4-(Difluoromethoxy)phenyl]sulfonylcyclopropyl]-6-[(3S)-3-methylmorpholin-4-yl]pyrimidin-2-yl]aniline

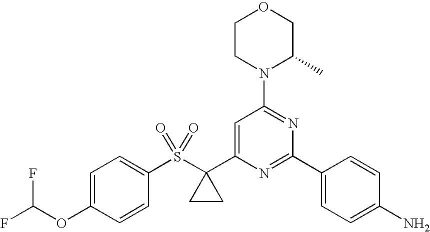

Trans-Dichlorobis(triphenylphosphine)palladium (II) (0.225 g, 0.32 mmol) was added to 2-chloro-4-[1-[4-(difluoromethoxy)phenyl]sulfonylcyclopropyl]-6-[(3S)-3-methylmorpholin-4-yl]pyrimidine (2.95 g, 6.41 mmol), 4-(4,4,5,5-tetramethyl-1,3,2-dioxaborolan-2-yl)aniline (1.476 g, 6.74 mmol) and 2M aqueous solution of sodium carbonate (16.04 mL, 32.07 mmol) in the DMF solution (150 mL) (DMF solution was 18% DMF, 82% of a 7:3:2 mixture of DME:H₂O:Ethanol) at RT under nitrogen. The resulting solution was stirred at 80° C. for 5 hours. The reaction was cooled and the reaction mixture diluted with ethyl acetate and water. The reaction mixture was extracted with ethyl acetate and the combined organics dried over MgSO₄, filtered and evaporated. The crude product was purified by flash silica chromatography, elution gradient 0 to 10% ethyl acetate in DCM, to give the desired material as a beige solid (1.35 g).

NMR Spectrum: ¹H NMR (400.132 MHz, DMSO-d₆) δ 1.18 (3H, d), 1.59 (2H, dd), 1.87 (2H, dd), 3.26 (1H, td), 3.50 (1H, td), 3.61 (1H, dd), 3.73 (1H, d), 3.95 (1H, dd), 4.10 (1H, d), 4.40 (1H, br s), 5.51 (2H, br s), 6.50 (2H, m), 6.53 (1H, s), 7.21-7.60 (1H, m), 7.35 (2H, m), 7.68 (2H, m), 7.83 (2H, m).

LCMS Spectrum: m/z (ES+)(M+H)+=517; HPLC tR=2.53 min.

### 2-Chloro-4-[1-[4-(difluoromethoxy)phenyl]sulfonylcyclopropyl]-6-[(3S)-3-methylmorpholin-4-yl]pyrimidine

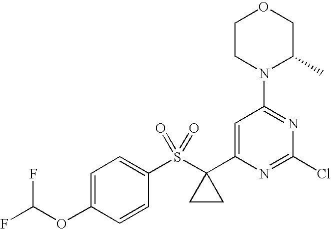

A solution of 50% w/v sodium hydroxide (14.2 mL, 354.96 mmol) was added portionwise to a stirred solution of 2-chloro-4-[[4-(difluoromethoxy)phenyl]sulfonylmethyl]-6-[(3S)-3-methylmorpholin-4-yl]pyrimidine (2.8 g, 6.45 mmol), tetrabutylammonium bromide (0.208 g, 0.65 mmol) and 1,2-dibromoethane (1.668 mL, 19.36 mmol) in toluene (150 mL) and the resulting suspension stirred at 60° C. for 6 hours. The reaction mixture was diluted with water (50 mL), and the washed sequentially with water (2×50 mL), and saturated brine (50 mL). The organic layer was dried over MgSO₄, filtered and evaporated to afford crude product. The crude product was purified by flash silica chromatography, elution gradient 0 to 10% ethyl acetate in DCM, to give the desired material as a colourless oil which solidified on standing (2.95 g).

NMR Spectrum: ¹H NMR (400.132 MHz, DMSO-d₆) δ 1.16 (3H, dd), 1.55 (2H, dd), 1.83 (2H, dd), 3.13 (1H, td), 3.40 (1H, td), 3.54 (1H, dd), 3.69 (1H, d), 3.90 (2H, dd), 4.23 (1H, br s), 6.68 (1H, s), 7.23-7.60 (1H, t), 7.37 (2H, d), 7.82 (2H, d).

LCMS Spectrum: m/z (ES+)(M+H)+=459; HPLC tR=2.49 min.

### 2-Chloro-4-[[4-(difluoromethoxy)phenyl]sulfonylmethyl]-6-[(3S)-3-methylmorpholin-4-yl]pyrimidine

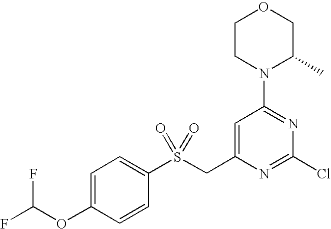

Sodium 4-(difluoromethoxy)benzenesulfinate (2.54 g, 11.03 mmol) was added portionwise to 2-chloro-4-(iodomethyl)-6-[(3S)-3-methylmorpholin-4-yl]pyrimidine (3.25 g, 9.19 mmol) in acetonitrile (125 mL) and the resulting suspension stirred at 80° C. for 6 hours. Additional sodium 4-(difluoromethoxy)benzenesulfinate (680mg, 3.39 mmol) was added in one portion and the suspension was stirred at 80° C. for a further 2 hours. The reaction mixture was concentrated and diluted with DCM (200 mL) and washed sequentially with 10% aqueous sodium thiosulfate solution (50 mL), water (50 mL), and saturated brine (50 mL). The organic layer was dried over MgSO₄, filtered and evaporated to afford crude product. The crude gum was triturated with diethyl ether to give the desired material as a beige solid (2.94 g).

NMR Spectrum: ¹H NMR (400.132 MHz, DMSO-d₆) δ 1.16 (3H, d), 3.15 (1H, td), 3.42 (1H, m), 3.57 (1H, dd), 3.71 (1H, d), 3.91 (1H, br s), 3.92 (1H, dd), 4.17 (1H, br s), 4.64 (2H, s), 6.68 (1H, s), 7.25-7.60 (1H, t), 7.41 (2H, m), 7.84 (2H, m).

LCMS Spectrum: m/z (ES+)(M+H)+=434; HPLC tR=2.29 min.

### Sodium 4-(difluoromethoxy)benzenesulfinate

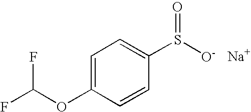

A solution of sodium sulfite (1.470 g, 11.66 mmol) in water (15 mL) was stirred at RT for 10 minutes. Sodium bicarbonate (1.960 g, 23.33 mmol) was added to the stirred solution. The resulting solution was stirred at 50° C. for 1 hour. 4-(Difluoromethoxy)benzene-1-sulfonyl chloride (2.83 g, 11.66 mmol) was added dropwise with caution to the solution and was stirred at 50° C. for 18 hours. The reaction mixture was evaporated to dryness and redissolved in methanol (15 mL). The suspension was allowed to stir at RT for 20 minutes. The suspension was filtered and the filtrate was evaporated to give a crude product that was triturated with isohexane to give the desired material as a cream solid (2.85 g).

NMR Spectrum: ¹H NMR (400.132 MHz, DMSO-d₆) δ 7.10 (2H, dd), 7.50 (2H, dd).

The preparation of 2-chloro-4-(iodomethyl)-6-[(3S)-3-methylmorpholin-4-yl]pyrimidine was described earlier.

The preparation of phenyl N-[4-[4-[1-(3,4-difluorophenyl)sulfonylcyclopropyl]-6-[(3S)-3-methylmorpholin-4-yl]pyrimidin-2-yl]phenyl]carbamate is described below.

### Phenyl N-[4-[4-[1-(3,4-difluorophenyl)sulfonylcyclopropyl]-6-[(3S)-3-methylmorpholin-4-yl]pyrimidin-2-yl]phenyl]carbamate

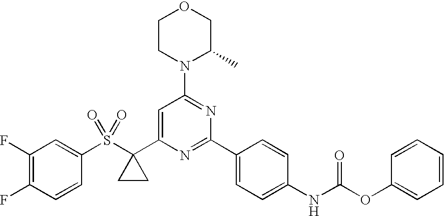

Phenyl chloroformate (0.206 mL, 1.64 mmol) was added dropwise to 4-[4-[1-(3,4-difluorophenyl)sulfonylcyclopropyl]-6-[(3S)-3-methylmorpholin-4-yl]pyrimidin-2-yl]aniline (800 mg, 1.64 mmol) and sodium hydrogen carbonate (138 mg, 1.64 mmol) in dioxane and the resulting suspension was stirred at RT for 6 hours. The reaction mixture was concentrated and diluted with DCM (50 mL), and washed sequentially with water (2×10 mL), and saturated brine (10 mL). The organic layer was dried over MgSO₄, filtered and evaporated to afford crude product. The crude solid was triturated with acetonitrile to give the desired material as a white solid (1.0 g).

NMR Spectrum: ¹H NMR (400.132 MHz, DMSO-d₆) δ 1.20 (3H, d), 1.63 (2H, dd), 1.94 (2H, dd), 3.17 (1H, td), 3.47 (1H, td), 3.61 (1H, dd), 3.75 (1H, d), 3.96 (1H, dd), 4.18 (1H, d), 4.50 (1H, br s), 6.71 (1H, s), 7.25 (2H, dt), 7.29 (1H, m), 7.45 (2H, dd), 7.54 (2H, d), 7.67 (2H, m), 7.91 (3H, dd), 10.40 (1H, s).

LCMS Spectrum: m/z (ES+)(M+H)+=607; HPLC tR=3.05 min.

### 4-[4-[1-(3,4-Difluorophenyl)sulfonylcyclopropyl]-6-[(3S)-3-methylmorpholin-4-yl]pyrimidin-2-yl]aniline

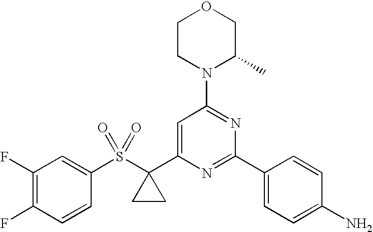

Trans-Dichlorobis(triphenylphosphine)palladium (II) (0.252 g, 0.36 mmol) was added to 2-chloro-4-[1-(3,4-difluorophenyl)sulfonylcyclopropyl]-6-[(3S)-3-methylmorpholin-4-yl]pyrimidine (3.09 g, 7.19 mmol), 4-(4,4,5,5-tetramethyl-1,3,2-dioxaborolan-2-yl)aniline (1.575 g, 7.19 mmol)and a 2M aqueous solution of sodium carbonate (17.97 mL, 35.94 mmol) in the DMF solution (DMF solution was 18% DMF, 82% of a 7:3:2 mixture of DME:H₂O:Ethanol) (150 mL) at RT under nitrogen. The resulting solution was stirred at 80° C. for 5 hours. The reaction was cooled and the mixture diluted with ethyl acetate and water. The reaction mixture was extracted with ethyl acetate and the combined organics dried over MgSO₄, filtered and evaporated. The crude product was purified by flash silica chromatography, elution gradient 0 to 5% ethyl acetate in DCM, to give a material which was further purified by ion exchange chromatography using an SCXcolumn, eluting with 7M ammonia in methanol, to give the desired material as a tan solid (1.94 g).

NMR Spectrum: ¹H NMR (400.132 MHz, DMSO-d₆) δ 1.18 (3H, d), 1.59 (2H, dd), 1.91 (2H, dd), 3.10 (1H, td), 3.45 (1H, td), 3.60 (1H, dd), 3.74 (1H, d), 3.95 (1H, dd), 4.13 (1H, m), 4.43 (1H, br s), 5.53 (2H, d), 6.50 (2H, m), 6.57 (1H, s), 7.64 (1H, m), 7.65 (3H, m), 7.89 (1H, m).

LCMS Spectrum: m/z (ES+)(M+H)+=487; HPLC tR=2.55 min.

### 2-Chloro-4-[1-(3,4-difluorophenyl)sulfonylcyclopropyl]-6-[(3S)-3-methylmorpholin-4-yl]pyrimidine

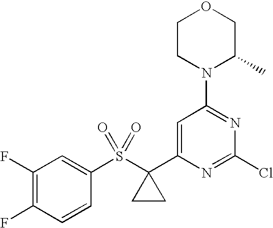

A solution of 50% w/v sodium hydroxide (17.32 mL, 433.10 mmol) was added portionwise to a stirred solution of 2-chloro-4-[(3,4-difluorophenyl)sulfonylmethyl]-6-[(3S)-3-methylmorpholin-4-yl]pyrimidine (3.18 g, 7.87 mmol), tetrabutylammonium bromide (0.254 g, 0.79 mmol) and 1,2-dibromoethane (2.036 mL, 23.62 mmol) in toluene (200 mL) and the resulting suspension stirred at 60° C. for 6 hours. The reaction mixture was diluted with water (50 mL), and washed sequentially with water (2×50 mL), and saturated brine (50 mL). The organic layer was dried over MgSO₄, filtered and evaporated to afford crude product. The crude product was purified by flash silica chromatography, elution gradient 0 to 20% ethyl acetate in DCM, to give the desired material as a white solid (3.09 g).

NMR Spectrum: ¹H NMR (400.132 MHz, DMSO-d₆) δ 1.16 (3H, d), 1.56 (2H, dd), 1.88 (2H, dd), 3.15 (1H, td), 3.40 (1H, td), 3.54 (1H, dd), 3.69 (1H, d), 3.91 (1H, dd), 3.97 (1H, d), 4.29 (1H, br s), 6.73 (1H, s), 7.65 (1H, m), 7.66 (1H, m), 7.92 (1H, ddd).

LCMS Spectrum: m/z (ES+)(M+H)+=430; HPLC tR=2.41 min.

### 2-Chloro-4-[(3,4-difluorophenyl)sulfonylmethyl]-6-[(³S)-³-methylmorpholin-4-yl]pyrimidine

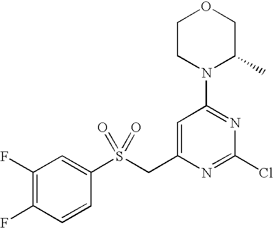

Sodium 3,4-difluorobenzenesulfinate (3.40 g, 16.97 mmol) was added portionwise to 2-chloro-4-(iodomethyl)-6-[(3S)-3-methylmorpholin-4-yl]pyrimidine (6 g, 16.97 mmol) in acetonitrile (200 mL) and the resulting suspension stirred at 80° C. for 6 hours. Additional sodium 3,4-difluorobenzenesulfinate (680mg, 3.39 mmol) was added in one portion and the suspension was stirred at 80° C. for a further 2 hours. The reaction mixture was concentrated and diluted with DCM (200 mL), and washed sequentially with water (2×50 mL), and saturated brine (50 mL). The organic layer was dried over MgSO₄, filtered and evaporated to afford crude product. The crude product was purified by flash silica chromatography, elution gradient 0 to 10% ethyl acetate in DCM, to give the desired material as a yellow solid (4.58 g).

NMR Spectrum: ¹H NMR (400.132 MHz, DMSO-d₆) δ 1.18 (3H, m), 3.10-3.20 (1H, td), 3.40-3.45 (1H, td), 3.55-3.60 (1H, dd), 3.70 (1H, d), 3.90-4.00 (2H, dd), 2H, dd), 4.20 (1H, br s), 4.70 (2H, s), 6.77 (1H, s), 7.66 (1H, m), 7.74 (1H, dt), 7.95 (1H, ddd).

LCMS Spectrum: m/z (ES+)(M+H)+=404; HPLC tR=2.24 min.

### Sodium 3,4-difluorobenzenesulfinate

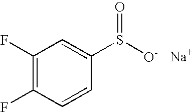

A solution of sodium sulfite (2.96 g, 23.52 mmol) in water (25 mL) was stirred at RT for 10 minutes. Sodium bicarbonate (3.95 g, 47.04 mmol) was added to the stirred solution. The resulting solution was stirred at 50° C. for 1 hour. 3,4-Difluorobenzene-1-sulfonyl chloride (5 g, 23.52 mmol) was added portionwise to the solution and was stirred at 50° C. for 18 hours. The reaction mixture was evaporated to dryness and redissolved in methanol (15 mL). The suspension was allowed to stir at RT for 20 minutes. The suspension was filtered and the filtrate was evaporated to afford the desired material as a white solid (5.30 g), which was dried overnight under vacuum and used without further purification.

NMR Spectrum: ¹H NMR (400.132 MHz, DMSO-d₆) δ 7.28 (1H, ddd), 7.37 (2H, m).

The preparation of 2-chloro-4-(iodomethyl)-6-[(3S)-3-methylmorpholin-4-yl]pyrimidine was described earlier

The preparation of phenyl N-[4-[4-[1-[1-(difluoromethyl)pyrazol-4-yl]sulfonylcyclopropyl]-6-[(3S)-3-methylmorpholin-4-yl]pyrimidin-2-yl]phenyl]carbamate is described below.

### Phenyl N-[4-[4-[1-[1-(difluoromethyl)pyrazol-4-yl]sulfonylcyclopropyl]-6-[(3S)-3-methylmorpholin-4-yl]pyrimidin-2-yl]phenyl]carbamate

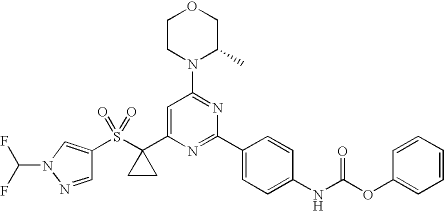

Phenyl chloroformate (0.018 mL, 0.14 mmol) was added dropwise to 4-[4-[1-[1-(difluoromethyl)pyrazol-4-yl]sulfonylcyclopropyl]-6-[(3S)-3-methylmorpholin-4-yl]pyrimidin-2-yl]aniline (70 mg, 0.14 mmol) and sodium hydrogen carbonate (11.99 mg, 0.14 mmol) in dioxane and the resulting suspension stirred at RT for 6 hours. The reaction mixture was concentrated and diluted with DCM (50 mL), and washed sequentially with water (2×10 mL), and saturated brine (10 mL). The organic layer was dried over MgSO₄, filtered and evaporated to afford crude product. The crude solid was triturated with diethyl ether:isohexane (20:80) to give the desired material as a white solid (80 mg).

NMR Spectrum: ¹H NMR (400.132 MHz, DMSO-d₆) δ 1.18 (3H, d), 1.59 (2H, m), 1.89 (2H, m), 3.15 (1H, td), 3.45 (1H, td), 3.63 (1H, dd), 3.75 (1H, d), 3.95 (1H, dd), 4.15 (1H, d), 4.50 (1H, br s), 6.75 (1H, m), 7.26 (3H, m), 7.45 (2H, m), 7.56 (2H, m), 7.70-8.00 (1H, t),8.00 (2H, m), 8.17 (1H, s), 8.93 (1H, s), 10.40 (1H, s).

LCMS Spectrum: m/z (ES+)(M+H)+=61 1; HPLC tR=2.83 min.

### 4-[4-[1-[1-(Difluoromethyl)pyrazol-4-yl]sulfonylcyclopropyl]-6-[(3S)-3-methylmorpholin-4-yl]pyrimidin-2-yl]aniline

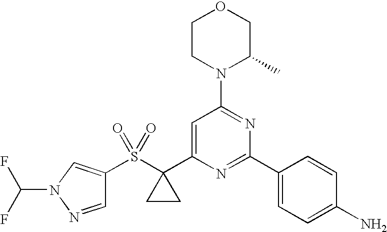

Trans-Dichlorobis(triphenylphosphine)palladium (II) (0.197 g, 0.28 mmol) was added to 2-chloro-4-[1-[1-(difluoromethyl)pyrazol-4-yl]sulfonylcyclopropyl]-6-[(3S)-3-methylmorpholin-4-yl]pyrimidine (2.44 g, 5.62 mmol), 4-(4,4,5,5-tetramethyl-1,3,2-dioxaborolan-2-yl)aniline (1.232 g, 5.62 mmol) and a 2M aqueous solution of sodium carbonate (14.06 mL, 28.12 mmol) in the DMF solution (DMF solution was 18% DMF, 82% of a 7:3:2 mixture of DME:H₂O:Ethanol) (150 mL) and the resulting solution stirred at 80° C. for 5 hours. The reaction mixture was partitioned between ethyl acetate and water, the layers separated and the aqueous layer extracted with ethyl acetate. The combined organics were dried over MgSO₄, filtered and evaporated. The crude product was purified by flash silica chromatography, elution gradient 0 to 5% ethyl acetate in DCM, to give the desired material as a tan solid (0.070 g).

LCMS Spectrum: m/z (ES+)(M+H)+=491; HPLC tR=2.23 min.

### 2-Chloro-4-[1-[1-(difluoromethyl)pyrazol-4-yl]sulfonylcyclopropyl]-6-[(3S)-3-methylmorpholin-4-yl]pyrimidine

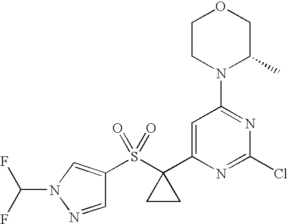

A solution of 50% w/v sodium hydroxide (16.18 mL, 404.59 mmol) was added portionwise to a stirred solution of 2-chloro-4-[[1-(difluoromethyl)pyrazol-4-yl]sulfonylmethyl]-6-[(3S)-3-methylmorpholin-4-yl]pyrimidine (3 g, 7.36 mmol), tetrabutylammonium bromide (0.237 g, 0.74 mmol) and 1,2-dibromoethane (1.902 mL, 22.07 mmol) in toluene (200 mL) and the resulting suspension stirred at RT for 6 hours. The reaction mixture was diluted with water (50 mL), and washed sequentially with water (2×50 mL), and saturated brine (50 mL). The organic layer was dried over MgSO₄, filtered and evaporated to afford crude product. The crude product was purified by flash silica chromatography, elution gradient 0 to 10% ethyl acetate in DCM, to give the desired material as a yellow oil (2.44 g) which solidified on standing.

NMR Spectrum: ¹H NMR (400.132 MHz, DMSO-d₆) δ 1.18 (3H, d), 1.57 (2H, dd), 1.82 (2H, dd), 3.16 (1H, td), 3.40 (1H, td), 3.56 (1H, dd), 3.70 (1H, d), 3.92 (1H, dd), 4.04 (1H, d), 4.30 (1H, br s), 6.78 (1H, s), 7.70-8.00 (1H, t), 8.19 (1H, s), 8.93 (1H, s).

LCMS Spectrum: m/z (ES+)(M+H)+=434; HPLC tR=2.15 min.

### 2-Chloro-4-[[1-(difluoromethyl)pyrazol-4-yl]sulfonylmethyl]-6-[(3S)-3-methylmorpholin-4-yl]pyrimidine

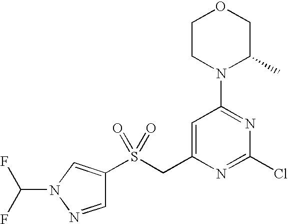

Sodium 1-(difluoromethyl)-1H-pyrazole-4-sulfinate (3.28 g, 16.09 mmol) was added portionwise to 2-chloro-4-(iodomethyl)-6-[(3S)-3-methylmorpholin-4-yl]pyrimidine (4.74 g, 13.41 mmol) in acetonitrile (150 mL) and the resulting suspension stirred at 80° C. for 6 hours. Additional sodium 1-(difluoromethyl)-1H-pyrazole-4-sulfinate (680 mg, 3.39 mmol) was added in one portion and the suspension was stirred at 80° C. for a further 2 hours. The reaction mixture was concentrated and diluted with DCM (200 mL), and washed sequentially with 10% aqueous sodium thiosulfate solution (50 mL), water (50 mL), and saturated brine (50 mL). The organic layer was dried over MgSO₄, filtered and evaporated to afford crude product. The crude residue was triturated with diethyl ether to give the desired material as a beige solid (4.50 g).

NMR Spectrum: ¹H NMR (400.132 MHz, DMSO-d₆) δ 1.19 (3H, d), 3.18 (1H, td), 3.43 (1H, td), 3.58 (1H, dd), 3.72 (1H, d), 3.93 (2H, dd), 4.21 (1H, br s), 4.67 (2H, s), 6.77 (1H, s), 7.70-8.05 (1H, t), 8.13 (1H, s), 8.87 (1H, s).

LCMS Spectrum: m/z (ES+)(M+H)+=408; HPLC tR=1.91 min.

### Sodium 1-(difluoromethyl)-1H-pyrazole-4-sulfinate

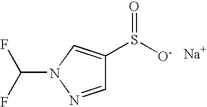

A solution of sodium sulfite (2.87 g, 22.81 mmol) in water (25 mL) was stirred at RT for 10 minutes. Sodium bicarbonate (3.83 g, 45.62 mmol) was added to the stirred solution. The resulting solution was stirred at 50° C. for 1 hour. 1-(Difluoromethyl)-1H-pyrazole-4-sulfonyl chloride (4.94 g, 22.81 mmol) was added dropwise with caution to the solution and was stirred at 50° C. for 18 hours. The reaction mixture was evaporated to dryness and redissolved in methanol (15 mL). The suspension was allowed to stir at RT for 20 minutes. The suspension was filtered and the filtrate evaporated to give a solid which was triturated with isohexane to give the desired material as a white solid (5.85 g).

NMR Spectrum: ¹H NMR (400.132 MHz, DMSO-d₆) δ 7.60 (1H, m), 7.74 (1H, s), 7.90 (1H, m).

The preparation of 2-chloro-4-(iodomethyl)-6-[(3S)-3-methylmorpholin-4-yl]pyrimidine was described earlier

### EXAMPLE 65

### 1-[4-[4-[1-(4-Fluorophenyl)sulfonylcyclobutyl]-6-[(3S)-3-methylmorpholin-4-yl]pyrimidin-2-yl]phenyl]-3-(1H-imidazol-2-ylmethyl)urea

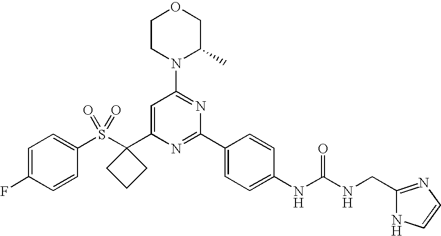

C-(1H-Imidazol-2-yl)-methylamine (17.73 mg, 0.18 mmol) was added in one portion to phenyl N-[4-[4-[1-(4-fluorophenyl)sulfonylcyclobutyl]-6-[(3S)-3-methylmorpholin-4-yl]pyrimidin-2-yl]phenyl]carbamate (100 mg, 0.17 mmol) and triethylamine (0.069 mL, 0.50 mmol) in NMP (2 mL) at RT and stirred for a period of 16 hours under air. The crude product was purified by preparative HPLC, eluting with decreasingly polar mixtures of water (containing 1% ammonia) and acetonitrile, to give the desired material as a cream solid (42 mg).

NMR Spectrum: ¹H NMR (399.902 MHz, DMSO-d₆) δ 1.21 (d, 3H), 1.83-1.93 (m, 1H), 2.04-2.15 (m, 1H), 2.75-2.82 (m, 2H), 3.02-3.11 (m, 2H), 3.16 (td, 1H), 3.49 (td, 1H), 3.64 (dd, 1H), 3.76 (d, 1H), 3.97 (dd, 1H), 4.15 (d, 1H), 4.32 (d, 2H), 4.44-4.54 (m, 1H), 6.55 (s, 1H), 6.60 (t, 1H), 6.80-6.90 (m, 1H), 7.00-7.08 (m, 1H), 7.29 (m, 2H), 7.40 (dt, 2H), 7.55 (ddd, 2H), 7.80 (d, 2H), 8.90 (s, 1H), 11.85 (s, 1H)

LCMS Spectrum: m/z (ESI+) (M+H)+=606.55; HPLC tR=2.09 min.

The compounds below were prepared in an analogous fashion from phenyl N-[4-[4-[1-(4-fluorophenyl)sulfonylcyclobutyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 65a | 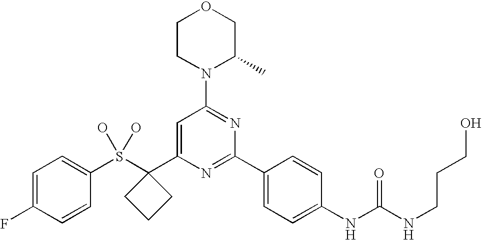 | 1-[4-[4-[1-(4-fluorophenyl)sulfonylcyclobutyl]-6-[(3S)-3-methylmorpholin-4-yl]pyrimidin-2-yl]phenyl]-3-(3-hydroxypropyl)urea | 584.5 | 2.15 |
| 65b | 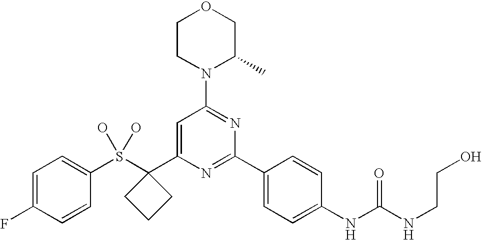 | 1-[4-[4-[1-(4-fluorophenyl)sulfonylcyclobutyl]-6-[(3S)-3-methylmorpholin-4-yl]pyrimidin-2-yl]phenyl]-3-(2-hydroxyethyl)urea | 570.5 | 2.05 |
| 65c | 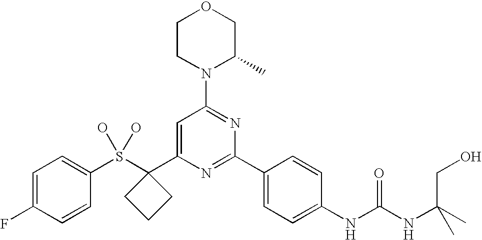 | 1-[4-[4-[1-(4-fluorophenyl)sulfonylcyclobutyl]-6-[(3S)-3-methylmorpholin-4-yl]pyrimidin-2-yl]phenyl]-3-(1-hydroxy-2-methylpropan-2-yl)urea | 598.6 | 2.33 |
| 65d | 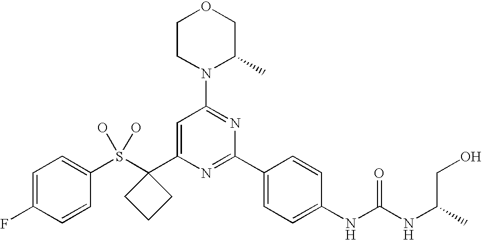 | 1-[4-[4-[1-(4-fluorophenyl)sulfonylcyclobutyl]-6-[(3S)-3-methylmorpholin-4-yl]pyrimidin-2-yl]phenyl]-3-[(2S)-1-hydroxypropan-2-yl]urea | 584.5 | 2.15 |
| 65e | 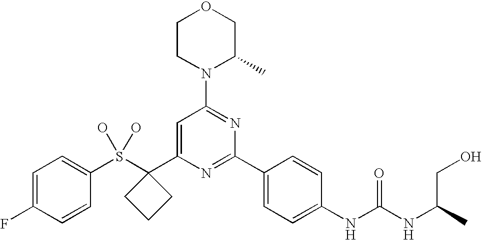 | 1-[4-[4-[1-(4-fluorophenyl)sulfonylcyclobutyl]-6-[(3S)-3-methylmorpholin-4-yl]pyrimidin-2-yl]phenyl]-3-[(2R)-1-hydroxypropan-2-yl]urea | 584.5 | 2.13 |

### EXAMPLE 65a

¹H NMR (399.902 MHz, DMSO-d₆) δ 1.21 (d, 3H), 1.57-1.63 (m, 2H), 1.83-1.94 (m, 1H), 2.05-2.15 (m, 1H), 2.75-2.82 (m, 2H), 3.01-3.09 (m, 2H), 3.17 (dd, 3H), 3.48 (dd, 3H), 3.64 (dd, 1H), 3.76 (d, 1H), 3.97 (dd, 1H), 4.10-4.19 (m, 1H), 4.48 (t, 2H), 6.18 (t, 1H), 6.54 (s, 1H), 7.29 (t, 2H), 7.38 (d, 2H), 7.55 (ddd, 2H), 7.78 (d, 2H), 8.68 (s, 1H)

### EXAMPLE 65b

¹H NMR (399.902 MHz, DMSO-d₆) δ 1.21 (d, 3H), 1.83-1.94 (m, 1H), 2.04-2.15 (m, 1H), 2.75-2.82 (m, 2H), 3.01-3.09 (m, 2H), 3.16-3.20 (m, 3H), 3.44-3.50 (m, 3H), 3.64 (dd, 1H), 3.76 (d, 1H), 3.97 (dd, 1H), 4.09-4.19 (m, 1H), 4.43-4.53 (m, 1H), 4.73 (t, 1H), 6.23 (t, 1H), 6.54 (s, 1H), 7.29 (t, 2H), 7.37 (d, 2H), 7.55 (ddd, 2H), 7.78 (d, 2H), 8.77 (s, 1H)

### EXAMPLE 65c

¹H NMR (399.902 MHz, DMSO-d₆) δ 1.21 (d, 3H), 1.25 (s, 6H), 1.83-1.94 (m, 1H), 2.05-2.15 (m, 1H), 2.73-2.83 (m, 2H), 3.01-3.09 (m, 2H), 3.12-3.20 (m, 1H), 3.39 (d, 2H), 3.49 (td, 1H), 3.64 (dd, 1H), 3.76 (d, 1H), 3.97 (dd, 1H), 4.10-4.18 (m, 1H), 4.43-4.53 (m, 1H), 4.96 (t, 1H), 5.98 (s, 1H), 6.54 (s, 1H), 7.27-7.35 (m, 4H), 7.53-7.57 (m, 2H), 7.77 (d, 2H), 8.70 (s, 1H)

### EXAMPLE 65d

¹H NMR (399.902 MHz, DMSO-d₆) δ 1.09 (d, 3H), 1.21 (d, 3H), 1.82-1.93 (m, 1H), 2.02-2.15 (m, 1H), 2.75-2.82 (m, 2H), 3.01-3.11 (m, 2H), 3.16 (td, 1H), 3.33-3.43 (m, 2H), 3.49 (td, 1H), 3.64 (dd, 1H), 3.69-3.73 (m, 1H), 3.76 (d, 1H), 3.97 (dd, 1H), 4.11-4.19 (m, 1H), 4.44-4.53 (m, 1H), 4.79 (t, 1H), 6.08 (d, 1H), 6.54 (s, 1H), 7.29 (ddd, 2H), 7.36 (d, 2H), 7.55 (ddd, 2H), 7.78 (d, 2H), 8.68 (s, 1H)

### EXAMPLE 65e

¹H NMR (399.902 MHz, DMSO-d₆) δ 1.09 (d, 3H), 1.21 (d, 3H), 1.83-1.94 (m, 1H), 2.05-2.15 (m, 1H), 2.75-2.82 (m, 2H), 3.01-3.09 (m, 2H), 3.16 (td, 1H), 3.33-3.43 (m, 2H), 3.49 (td, 1H), 3.64 (dd, 1H), 3.69-3.73 (m, 1H), 3.76 (d, 1H), 3.97 (dd, 1H), 4.10-4.18 (m, 1H), 4.44-4.53 (m, 1H), 4.79 (t, 1H), 6.08 (d, 1H), 6.54 (s, 1H), 7.29 (ddd, 2H), 7.36 (d, 2H), 7.55 (ddd, 2H), 7.78 (d, 2H), 8.68 (s, 1H)

The preparation of phenyl N-[4-[4-[1-(4-fluorophenyl)sulfonylcyclobutyl]-6-[(3S)-3-methylmorpholin-4-yl]pyrimidin-2-yl]phenyl]carbamate is described below:

### Phenyl N-[4-[4-[1-(4-fluorophenyl)sulfonylcyclobutyl]-6-[(3S)-3-methylmorpholin-4-yl]pyrimidin-2-yl]phenyl]carbamate

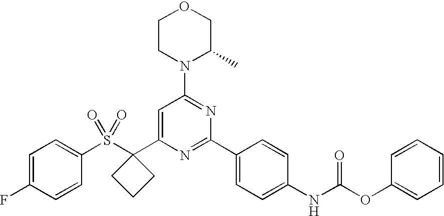

Phenyl chloroformate (0.285 mL, 2.27 mmol) was added dropwise to 4-[4-[1-(4-fluorophenyl)sulfonylcyclobutyl]-6-[(3S)-3-methylmorpholin-4-yl]pyrimidin-2-yl]aniline (1.095 g, 2.27 mmol) and sodium hydrogencarbonate (0.191 g, 2.27 mmol) in dioxane (20 mL) and the resulting suspension was stirred at RT for 3 hours. The reaction mixture was evaporated and DCM (50 mL) added and washed sequentially with water (2×20 mL), and saturated brine (20 mL). The organic layer was dried over MgSO₄, filtered and evaporated to afford desired material as a dry film (1.4 g).

NMR Spectrum: ¹H NMR (399.902 MHz, DMSO-d₆) δ 1.22 (d, 3H), 1.84-1.95 (m, 1H), 2.05-2.16 (m, 1H), 2.74-2.86 (m, 2H), 3.02-3.12 (m, 2H), 3.17 (td, 1H), 3.45-3.55 (m, 1H), 3.61-3.69 (m, 1H), 3.71-3.85 (m, 1H), 3.93-4.03 (m, 1H), 4.11-4.24 (m, 1H), 4.45-4.58 (m, 1H), 7.22-7.35 (m, 5H), 7.42-7.49 (m, 2H), 7.50-7.60 (m, 4H), 7.88 (d, 2H), 10.40 (s, 1H)

LCMS Spectrum: m/z (ESI+) (M+H)+=603.17; HPLC tR=3.15 min.

### 4-[4-[1-(4-Fluorophenyl)sulfonylcyclobutyl]-6-[(3S)-3-methylmorpholin-4-yl]pyrimidin-2-yl]aniline

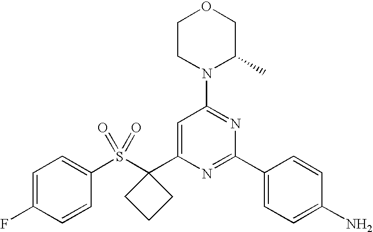

Bis(triphenylphosphine)palladium(II) chloride (0.086 g, 0.12 mmol) was added in one portion to 2-chloro-4-[1-(4-fluorophenyl)sulfonylcyclobutyl]-6-[(3S)-3-methylmorpholin-4-yl]pyrimidine (1.046 g, 2.46 mmol), 4-(4,4,5,5-tetramethyl-1,3,2-dioxaborolan-2-yl)aniline (0.538 g, 2.46 mmol) and sodium carbonate (6.14 mL, 12.28 mmol) in a DMF:DME:water:ethanol solution and the reaction mixture thoroughly degassed and stirred at 80° C. for 3 hours under a nitrogen atmosphere. The reaction mixture was evaporated to dryness and redissolved in DCM (50 mL), and washed sequentially with water (50 mL) and saturated brine (50 mL). The organic layer was dried over MgSO₄, filtered and evaporated to afford crude product. The crude product was purified by flash silica chromatography, elution gradient 30 to 100% ethyl acetate in isohexane, to afford desired material as a colourless dry film (1.1 g).

NMR Spectrum: ¹H NMR (399.902 MHz, DMSO-d₆) δ 1.20 (d, 3H), 1.82-1.95 (m, 1H), 2.03-2.14 (m, 1H), 2.71-2.82 (m, 2H), 2.98-3.08 (m, 2H), 3.13 (td, 1H), 3.48 (td, 1H), 3.63 (dd, 1H), 3.75 (d, 1H), 3.93-3.99 (m, 1H), 4.07-4.15 (m, 1H), 4.41-4.49 (m, 1H), 5.49 (d, 1H), 6.44 (s, 1H), 6.49 (d, 2H), 7.28 (ddd, 2H), 7.54 (ddd, 2H), 7.62 (d, 2H)

LCMS Spectrum: m/z (ESI+) (M+H)+=483.27; HPLC tR=2.39 min.

### 2-Chloro-4-[1-(4-fluorophenyl)sulfonylcyclobutyl]-6-[(3S)-3-methylmorpholin-4-yl]pyrimidine

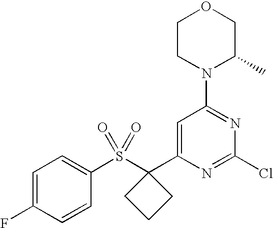

Sodium hydroxide (50% w/w solution) (16.91 g, 422.79 mmol) was added to 2-chloro-4-[(4-fluorophenyl)sulfonylmethyl]-6-[(3S)-3-methylmorpholin-4-yl]pyrimidine (2.966 g, 7.69 mmol), 1,3-dibromopropane (2.341 mL, 23.06 mmol) and tetrabutylammonium bromide (0.248 g, 0.77 mmol) in toluene (150 mL) and the resulting suspension stirred at 45° C. for 1 hour. The organics were washed with water twice, dried over MgSO₄, filtered and evaporated. The crude product was purified by flash silica chromatography, elution gradient 10 to 30% ethyl acetate in DCM, to afford desired material as a colourless dry film (1.055 g).

NMR Spectrum: ¹H NMR (399.902 MHz, DMSO-d₆) δ 1.18 (d, 3H), 1.81-1.92 (m, 1H), 2.01-2.12 (m, 1H), 2.65-2.72 (m, 2H), 2.91-3.01 (m, 2H), 3.14 (td, 1H), 3.42 (td, 1H), 3.57 (dd, 1H), 3.71 (d, 1H), 3.88-4.00 (m, 2H), 4.23-4.40 (m, 1H), 6.56 (s, 1H), 7.38-7.44 (m, 2H), 7.54-7.60 (m, 2H)

LCMS Spectrum: m/z (ESI+) (M+H)+=426.06; HPLC tR=2.52 min.

The preparation of 2-chloro-4-[(4-fluorophenyl)sulfonylmethyl]-6-[(3S)-3-methylmorpholin-4-yl]pyrimidine was described earlier.

### EXAMPLE 66

### 3-(2-Hydroxyethyl)-1-[4-[4-[(3S)-3-methylmorpholin-4-yl]-6-[1-(1,3-thiazol-2-ylsulfonyl)cyclopropyl]pyrimidin-2-yl]phenyl]urea

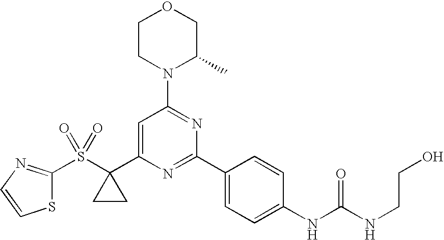

Ethanolamine (0.052 mL, 0.87 mmol) was added in one portion to phenyl N-[4-[4-[(3S)-3-methylmorpholin-4-yl]-6-[1-(1,3-thiazol-2-ylsulfonyl)cyclopropyl]pyrimidin-2-yl]phenyl]carbamate (100 mg, 0.17 mmol) and triethylamine (0.072 mL, 0.52 mmol) in NMP (2 mL) and warmed to 55° C. over a period of 16 hours under air. The crude reaction mixture was purified by preparative HPLC, eluting with decreasingly polar mixtures of water (containing 1% ammonia) and acetonitrile, to afford the desired material as a colourless dry film. (84 mg)

NMR Spectrum: ¹H NMR (399.902 MHz, DMSO-d₆) δ 1.21 (d, 3H), 1.77-1.82 (m, 2H), 1.98-2.02 (m, 2H), 3.14-3.21 (m, 3H), 3.44-3.50 (m, 3H), 3.62 (dd, 1H), 1H), 3.76 (d, 1H), 3.97 (dd, 1H), 4.10-4.21 (m, 1H), 4.40-4.51 (m, 1H), 4.74 (t, 1H), 6.25 (t, 1H), 6.74 (s, 1H), 7.40 (d, 2H), 7.85 (d, 2H), 8.24 (d, 1H), 8.29 (d, 1H), 8.78 (s, 1H)

LCMS Spectrum: m/z (ESI+) (M+H)+=545.75; HPLC tR=1.79 min.

The compounds below were prepared in an analogous fashion from phenyl N-[4-[4-[(3S)-3-methylmorpholin-4-yl]-6-[1-(1,3-thiazol-2-ylsulfonyl)cyclopropyl]pyrimidin-2-yl]phenyl]carbamate and the appropriate amine.

| | | | LCMS | Retention |
|---|---|---|---|---|
| Example | Structure | NAME | MH+ | time (min) |
| 66a | 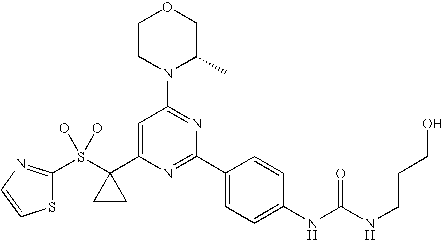 | 3-(3-hydroxypropyl)-1-[4-[4-[(3S)-3-methylmorpholin-4-yl]-6-[1-(1,3-thiazol-2-ylsulfonyl)cyclopropyl]pyrimidin-2-yl]phenyl]urea | 559.8 | 1.84 |
| 66b | 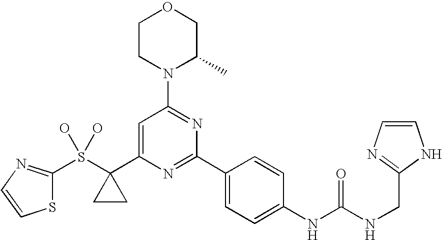 | 3-(1H-imidazol-2-ylmethyl)-1-[4-[4-[(3S)-3-methylmorpholin-4-yl]-6-[1-(1,3-thiazol-2-ylsulfonyl)cyclopropyl]pyrimidin-2-yl]phenyl]urea | 581.8 | 1.30 |

### EXAMPLE 66a

¹H NMR (399.902 MHz, DMSO-d₆) δ 1.21, 1.60, 1.77-1.82, 1.97-2.03, 3.14-3.21, 3.44-3.50, 3.62, 3.76, 3.97, 4.12-4.20, 4.40-4.52, 4.49, 6.19, 6.74, 7.40, 7.85, 8.23, 8.28, 8.68

### EXAMPLE 66b

¹H NMR (399.902 MHz, DMSO-d₆) δ 1.21 (d, 3H), 1.77-1.82 (m, 2H), 1.97-2.03 (m, 2H), 3.14-3.21 (m, 1H), 3.47 (td, 1H), 3.62 (dd, 1H), 3.76 (d, 1H), 3.97 (dd, 1H), 4.11-4.21 (m, 1H), 4.32 (d, 2H), 4.39-4.54 (m, 1H), 6.62 (t, 1H), 6.74 (s, 1H), 6.88-7.01 (m, 2H), 7.43 (d, 2H), 7.87 (d, 2H), 8.23 (d, 1H), 8.28 (d, 1H), 8.90 (s, 1H), 11.85 (s, 1H)

The preparation of phenyl N-[4-[4-[(3S)-3-methylmorpholin-4-yl]-6-[1-(1,3-thiazol-2-ylsulfonyl)cyclopropyl]pyrimidin-2-yl]phenyl]carbamate is described below:

### Phenyl N-[4-[4-[(3S)-3-methylmorpholin-4-yl]-6-[1-(1,3-thiazol-2-ylsulfonyl)cyclopropyl]pyrimidin-2-yl]phenyl]carbamate

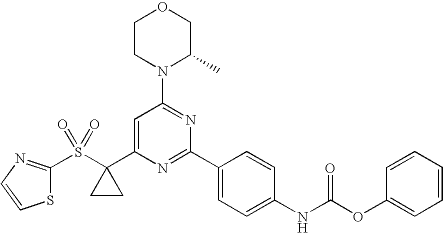

Phenyl chloroformate (0.123 mL, 0.98 mmol) was added dropwise to 4-[4-[(3S)-3-methylmorpholin-4-yl]-6-[1-(1,3-thiazol-2-ylsulfonyl)cyclopropyl]pyrimidin-2-yl]aniline (450 mg, 0.98 mmol) and sodium hydrogencarbonate (83 mg, 0.98 mmol) in dioxane and the resulting suspension stirred at RT for 3 hours. The reaction mixture was evaporated and DCM (50 mL) added and washed sequentially with water (2×20 mL), and saturated brine (20 mL). The organic layer was dried over MgSO₄, filtered and evaporated to afford desired product as a dry film (522 mg).

NMR Spectrum: ¹H NMR (399.902 MHz, DMSO-d₆) δ 1.22 (d, 3H), 1.78-1.84 (m, 2H), 1.98-2.04 (m, 2H), 3.19 (td, 1H), 3.48 (td, 1H), 3.63 (dd, 1H), 3.76 (d, 1H), 3.97 (dd, 1H), 4.12-4.24 (m, 1H), 4.40-4.52 (m, 1H), 6.79 (s, 1H), 7.23-7.32 (m, 3H), 7.46 (t, 2H), 7.55 (d, 2H), 7.95 (d, 2H), 8.24 (d, 1H), 8.29 (d, 1H), 10.41 (s, 1H)

LCMS Spectrum: m/z (ESI+) (M+H)+=578.04; HPLC tR=2.83 min.

### 4-[4-[(3S)-3-Methylmorpholin-4-yl]-6-[1-(1,3-thiazol-2-ylsulfonyl)cyclopropyl]pyrimidin-2-yl]aniline

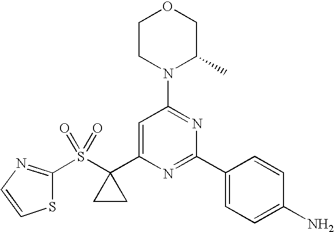

Bis(triphenylphosphine)palladium(II) chloride (0.139 g, 0.20 mmol) was added in one portion to 2-chloro-4-[(3S)-3-methylmorpholin-4-yl]-6-[1-(1,3-thiazol-2-ylsulfonyl)cyclopropyl]pyrimidine (1.59 g, 3.97 mmol), 4-(4,4,5,5-tetramethyl-1,3,2-dioxaborolan-2-yl)aniline (0.869 g, 3.97 mmol) and sodium carbonate (9.92 mL, 19.83 mmol) in a DMF, DME, water and ethanol solution at RT under nitrogen. The reaction mixture was thoroughly degassed and was stirred at 80° C. for 3 hours. The reaction mixture was evaporated to dryness and redissolved in DCM (50 mL), and washed sequentially with water (50 mL) and saturated brine (50 mL). The organic layer was dried over MgSO₄, filtered and evaporated to afford crude product. The crude product was purified by flash silica chromatography, elution gradient 30 to 100% ethyl acetate in isohexane, to afford desired material as a beige dry film (1.430 g).

NMR Spectrum: ¹H NMR (399.902 MHz, DMSO-d₆) δ 1.16-1.21 (m, 3H), 1.75-1.79 (m, 2H), 1.96-2.01 (m, 2H), 3.14 (td, 1H), 3.46 (td, 1H), 3.61 (dd, 1H), 3.75 (d, 1H), 3.96 (dd, 1H), 4.07-4.17 (m, 1H), 4.35-4.48 (m, 1H), 5.50-5.55 (m, 1H), 6.52 (d, 2H), 6.63 (s, 1H), 7.69 (d, 2H), 8.22 (d, 1H), 8.27 (d, 1H)

LCMS Spectrum: m/z (ESI+) (M+H)+=458.16; HPLC tR=1.68 min.

The preparation of 2-chloro-4-[(3S)-3-methylmorpholin-4-yl]-6-[1-(1,3-thiazol-2-ylsulfonyl)cyclopropyl]pyrimidine was described earlier.

### EXAMPLE 67

3-(2-Hydroxyethyl)-1-[4-[4-[(3S)-3-methylmorpholin-4-yl]-6-[1-(1,3-thiazol-2-ylsulfonyl)cyclopropyl]pyrimidin-2-yl]phenyl]thiourea

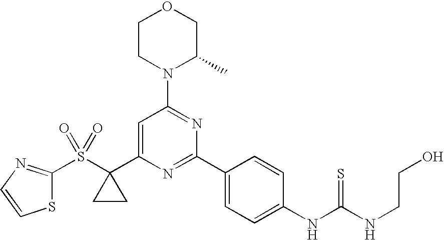

A solution of 1,1′-thiocarbonyldiimidazole (50.6 mg, 0.28 mmol) in DCM (2 mL) was added to a stirred solution 4-[4-[(3S)-3-methylmorpholin-4-yl]-6-[1-(1,3-thiazol-2-ylsulfonyl)cyclopropyl]pyrimidin-2-yl]aniline (100 mg, 0.22 mmol) in THF (1 mL) and DCM (2 mL) at RT, over a period of 2 minutes under nitrogen. The resulting solution was stirred at RT for 2 hours. Triethylamine (0.030 mL, 0.22 mmol) and ethanolamine (0.066 mL, 1.09 mmol) were added to the reaction mixture. The resulting solution was stirred at RT for 60 hours. The reaction mixture was evaporated to dryness and redissolved in acetonitrile (2 mL), filtered and purified by preparative HPLC, eluting with decreasingly polar mixtures of water (containing 1% ammonia) and acetonitrile, to afford desired material as a beige solid (88 mg).

NMR Spectrum: ¹H NMR (399.902 MHz, DMSO-d₆) δ 1.22 (d, 3H), 1.78-1.84 (m, 2H), 1.98-2.03 (m, 2H), 3.19 (td, 1H), 3.48 (td, 1H), 3.54-3.60 (m, 3H), 3.63 (dd, 1H), 3.76 (d, 1H), 3.97 (dd, 1H), 4.16 (d, 1H), 4.41-4.53 (m, 1H), 4.78-4.88 (m, 1H), 6.79 (s, 1H), 7.54 (d, 2H), 7.82-7.89 (m, 1H), 7.92 (dt, 2H), 8.24 (d, 1H), 8.28 (d, 1H), 9.78 (s, 1H)

LCMS Spectrum: m/z (ESI+) (M+H)+=561.82; HPLC tR=2.12 min.

The preparation of 4-[4-[(3S)-3-methylmorpholin-4-yl]-6-[1-(1,3-thiazol-2-ylsulfonyl)cyclopropyl]pyrimidin-2-yl]aniline was described earlier.

### EXAMPLE 68

### 3-Cyclopropyl-1-[4-[4-[(3S)-3-methylmorpholin-4-yl]-6-[1-(4-methylphenyl)sulfonylcyclobutyl]pyrimidin-2-yl]phenyl]urea

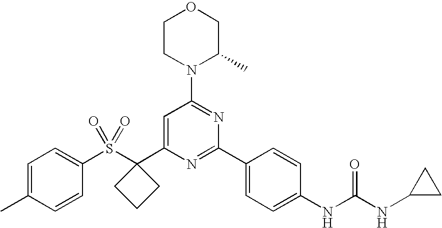

Triethylamine (0.057 mL, 0.41 mmol) was added to phenyl N-[4-[4-[(3S)-3-methylmorpholin-4-yl]-6-[1-(4-methylphenyl)sulfonylcyclobutyl]pyrimidin-2-yl]phenyl]carbamate (82 mg, 0.14 mmol) and cyclopropylamine (0.047 mL, 0.68 mmol) in NMP (2 mL) and the resulting solution stirred at 50° C. overnight. The crude product was purified by preparative HPLC, eluting with decreasingly polar mixtures of water (containing 1% ammonia) and acetonitrile, to give the desired material as a white solid (43 mg).

NMR Spectrum: ¹H NMR (400.132 MHz, DMSO-d₆) δ 0.40-0.44(2H, m), 0.63-0.67(2H, m), 1.19-1.20(3H, d), 1.81-1.92(1H, m), 2.02-2.13(1H, m), 2.27(3H, s), 2.54-2.59(1H, m), 2.73-2.79(2H, m), 2.99-3.07(2H, m), 3.09-3.17(1H, td), 3.45-3.51(1H, td), 3.62-3.65(1H, dd), 3.74-3.77(1H, d), 3.95-3.98(1H, dd), 4.07-4.10(1H, d), 4.44(1H, bs), 6.40-6.41(1H, d), 6.45(1H, s), 7.24-7.26(2H, d), 7.35-7.40(4H, m), 7.76-7.78(2H, d), 8.49(1H, s).

LCMS Spectrum: m/z (ESI+)(M+H)+562 =HPLC tR=2.60 min.

The following compound was made in an analogous fashion using the appropriate amine.

| | | | | Reten- |
|---|---|---|---|---|
| | | | | tion |
| | | | LCMS | time |
| Example | Structure | NAME | MH+ | (min) |
| 68a | 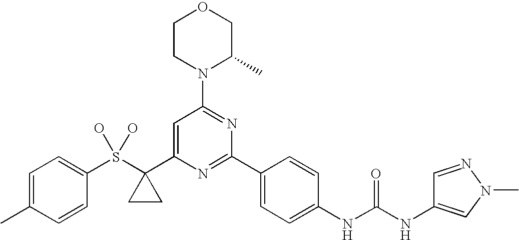 | 1-[4-[4-[(3S)-3-methylmorpholin-4-yl]-6-[1-(4-methylphenyl)sulfonyl-cyclobutyl]pyrimidin-2-yl]phenyl]-3-(1-methyl-pyrazol-4-yl)urea | 601 | 2.44 |

### EXAMPLE 68a

¹H NMR (400.132 MHz, DMSO-d₆) δ 1.19-1.21(3H, d), 1.83-1.90(1H, m), 2.04-2.12(1H, m), 2.28(3H, s), 2.73-2.80(2H, m), 3.00-3.07(2H, m), 3.10-3.15(1H, td), 3.45-3.52(1H, td), 3.62-3.66(1H, dd), 3.75-3.78(1H, d), 3.79(3H, s), 3.95-3.99(1H, td), 3.45-4.11(1H, d), 4.45(1H, bs), 6.46(1H, s), 7.24-7.26(2H, d), 7.35-7.38(3H, m), 7.42-7.44(2H, d), 7.77(1H, s), 7.79-7.81(2H, d), 8.35(1H, s), 8.79(1H, s).

### Phenyl N-[4-[4-[(3S)-3-methylmorpholin-4-yl]-6-[1-(4-methylphenyl)sulfonylcyclobutyl]pyrimidin-2-yl]phenyl]carbamate

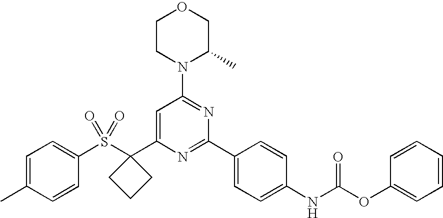

Phenyl chloroformate (0.034 mL, 0.27 mmol) was added to 4-[4-[(3S)-3-methylmorpholin-4-yl]-6-[1-(4-methylphenyl)sulfonylcyclobutyl]pyrimidin-2-yl]aniline (129 mg, 0.27 mmol)and sodium hydrogen carbonate (34.0 mg, 0.40 mmol) in dioxane (10 mL). The resulting suspension was stirred at RT for 5 hours. The reaction mixture was evaporated to dryness and redissolved in ethyl acetate(125 mL), and washed sequentially with water (125 mL), and saturated brine (50 mL). The organic layer was dried over MgSO₄, filtered and evaporated to afford the desired material (182 mg).

NMR Spectrum: ¹H NMR (400.132 MHz, CDCl3) δ 1.34 (3H, d), 1.87-1.98 (1H, m), 2.19-2.30 (4H, m), 2.72-2.81 (2H, m), 3.15-3.25 (2H, m), 3.30 (1H, td), 3.63 (1H, td), 3.70 (1H, s), 3.72-3.82 (3H, m), 4.03-4.16 (3H, m), 4.46 (1H, q), 6.56 (1H, s), 7.01 (21H, s), 7.07 (2H, d), 7.19-7.27 (3H, m, obscured by CDCL3 peak), 7.35-7.42 (6H, m), 7.91 (2H, d).

LCMS Spectrum: m/z (ES+)(M+H)+=599; HPLC tR=3.25 min.

### 4-[4-[(3S)-3-Methylmorpholin-4-yl]-6-[ 1-(4-methylphenyl)sulfonylcyclobutyl]pyrimidin-2-yl]aniline

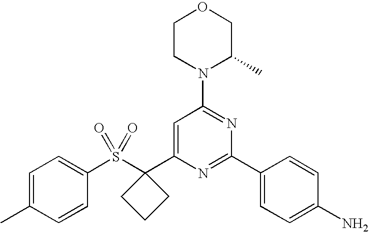

Bis(triphenylphosphine)palladium(II) chloride (0.131 g, 0.19 mmol) was added in one portion to a mixture of 2-chloro-4-[(3S)-3-methylmorpholin-4-yl]-6-[1-(4-methylphenyl)sulfonylcyclobutyl]pyrimidine and 4-[1-(benzenesulfonyl)cyclobutyl]-2-chloro-6-[(3S)-3-methylmorpholin-4-yl]pyrimidine (1.522 g), 4-(4,4,5,5-tetramethyl-1,3,2-dioxaborolan-2-yl)aniline (0.817 g, 3.73 mmol) and a solution of sodium carbonate (9.33 mL, 18.66 mmol) in DMF (18 mL), DME (47.8 mL), water (20.5 mL)and ethanol (13.5 mL) and the resulting solution stirred at 80° C. for 2 hours. The reaction mixture was concentrated in vacuo to remove the ethanol then the reaction mixture was acidified with 2M hydrochloric acid. The resulting suspension was purified by ion exchange chromatography using an SCX column, eluting with 7M ammonia in methanol, followed by preparative HPLC, to give the desired material as an off white solid (0.136 g).

NMR Spectrum:¹H NMR (400.132 MHz, CDCl₃) δ 1.32 (3H, d), 1.85-1.97 (1H, m), 2.17-2.29 (4H, m), 2.71-2.81 (2H, m), 3.11-3.33 (3H, m), 3.61 (1H, t), 3.74-3.90 (4H, m), 4.03 (1H, d), 4.09-4.15 (1H, m), 4.44 (1H, q), 6.48 (1H, s), 6.57 (2H, d), 7.07 (2H, d), 7.36 (2H, d), 7.75 (2H, d).

LCMS Spectrum: m/z (ES+)(M+H)+=479; HPLC tR=2.23 min.

### Mixture of 2-chloro-4-[(3S)-3-methylmorpholin-4-yl]-6-[1-(4-methylphenyl)sulfonylcyclobutyl]pyrimidine and 4-[1-(benzenesulfonyl)cyclobutyl]-2-chloro-6-[(3S)-3-methylmorpholin-4-yl]pyrimidine

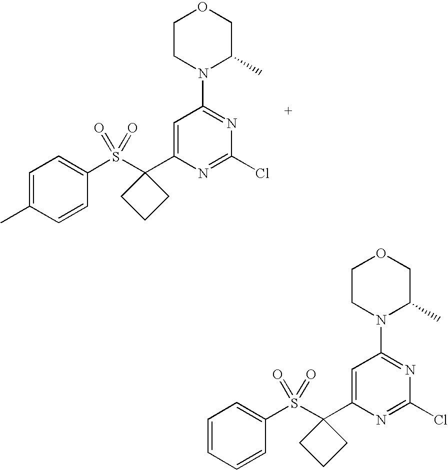

Sodium hydroxide (50% w/w solution) (52.4 g, 654.89 mmol) was added to a mixture of 2-chloro-4-[(3S)-3-methylmorpholin-4-yl]-6-[(4-methylphenyl)sulfonylmethyl]pyrimidine and 4-(benzenesulfonylmethyl)-2-chloro-6-[(3 S)-3-methylmorpholin-4-yl]pyrimidine (4.38 g), 1,3-dibromopropane (3.63 mL, 35.72 mmol) and tetrabutylammonium bromide (0.384 g, 1.19 mmol) in toluene (270 mL). The resulting suspension was stirred at 45° C. for 1 hour. The reaction mixture was diluted with water (300 mL). The organic layer was dried over MgSO₄, filtered and evaporated to afford crude product. The crude product was purified by flash silica chromatography, elution gradient 0 to 40% ethyl acetate in DCM, to give a white solid (1.522 g) which appeared to be a mixture of 2-chloro-4-[(3S)-3-methylmorpholin-4-yl]-6-[1-(4-methylphenyl)sulfonylcyclobutyl]pyrimidine (12%) and 4-[1-(benzenesulfonyl)cyclobutyl]-2-chloro-6-[(3S)-3-methylmorpholin-4-yl]pyrimidine (83%). The mixture was taken through into the next step with no further purification.

LCMS Spectrum: m/z (ES+)(M+H)+=422; HPLC tR=2.59 min.

LCMS Spectrum: m/z (ES+)(M+H)+=408; HPLC tR=2.45 min.

### Mixture of 2-chloro-4-[(3S)-3-methylmorpholin-4-yl]-6-[(4-methylphenyl)sulfonylmethyl]pyrimidine and ⁴-(benzenesulfonylmethyl)-2-chloro-6-[(3S)-3-methylmorpholin-4-yl]pyrimidine

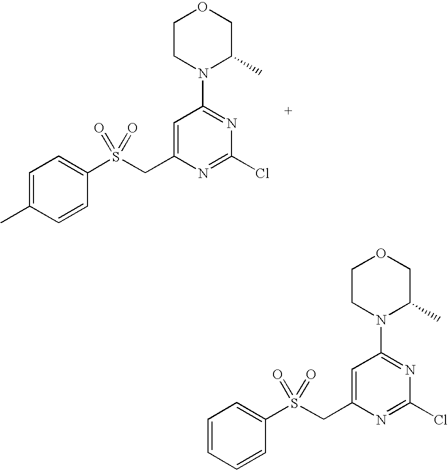

Benzenesulfinic acid, sodium salt (3.66 g, 22.32 mmol) was added to a mixture of 2-chloro-4-[(3S)-3-methylmorpholin-4-yl]-6-[(4-methylphenyl)sulfonylmethyl]pyrimidine and 2-chloro-4-(chloromethyl)-6-[(3S)-3-methylmorpholin-4-yl]pyrimidine (5.85 g) in acetonitrile (200 mL) under argon. The resulting suspension was stirred at reflux for 18 hours. Additional benzenesulfinic acid, sodium salt (1.2 g, 7.31 mmol) and sodium iodide (0.335 g, 2.23 mmol) were added and the suspension was stirred at reflux for a further 24 hours. The reaction mixture was evaporated to dryness, redissolved in DCM (500 mL) and washed with water (250 mL). The organic layer was dried over MgSO₄, filtered and evaporated. The crude material was purified by flash silica chromatography, elution gradient 0 to 50% ethyl acetate in DCM, to give a white solid (4.38 g) which appeared to be a mixture of 2-chloro-4-[(3S)-3-methylmorpholin-4-yl]-6-[(4-methylphenyl)sulfonylmethyl]pyrimidine (7%) and 4-(benzenesulfonylmethyl)-2-chloro-6-[(3 S)-3-methylmorpholin-4-yl]pyrimidine (93%). The mixture was taken through into the next step with no further purification.

LCMS Spectrum: m/z (ES+)(M+H)+=382; HPLC tR=2.23 min.

LCMS Spectrum: m/z (ES+)(M+H)+=368; HPLC tR=2.06 min.

### Mixture of 2-chloro-4-[(3S)-3-methylmorpholin-4-yl]-6-[(4-methylphenyl)sulfonylmethyl]pyrimidine and 2-chloro-4-(chloromethyl)-6-[(3S)-3-methylmorpholin-4-yl]pyrimidine

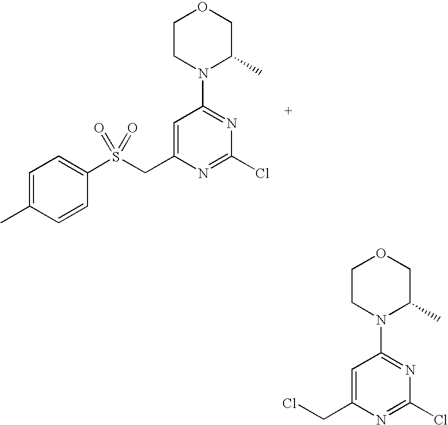

A solution of p-toluenesulfonyl chloride (23.47 g, 123.11 mmol) in DCM (50 mL) was added dropwise to a stirred solution of [2-chloro-6-[(3S)-3-methylmorpholin-4-yl]pyrimidin-4-yl]methanol (20 g, 82.07 mmol) and DIPEA (21.44 mL, 123.11 mmol) in DCM (200 mL) cooled to 5° C., over a period of 1 hour under nitrogen. The resulting solution was stirred at 5° C. for 72 hours and then at reflux for 24 hours. The reaction mixture was washed with water and the organic layer was dried over MgSO₄, filtered and evaporated to afford a brown gum which appeared to be a mixture of 2-chloro-4-[(3S)-3-methylmorpholin-4-yl]-6-[(4-methylphenyl)sulfonylmethyl]pyrimidine (11%) and 2-chloro-4-(chloromethyl)-6-[(3S)-3-methylmorpholin-4-yl]pyrimidine (87%). The mixture was taken through into the next step with no further purification.

LCMS Spectrum: m/z (ES+)(M+H)+=398; HPLC tR=2.52 min.

LCMS Spectrum: m/z (ES+)(M+H)+=262; HPLC tR=1.97 min.

The preparation of [2-chloro-6-[(3S)-3-methylmorpholin-4-yl]pyrimidin-4-yl]methanol was described earlier.

### EXAMPLE 69

### 1-[4-[4-[1-(Benzenesulfonyl)cyclopropyl]-6-[(3R)-3-methylmorpholin-4-yl]pyrimidin-2-yl]phenyl]-3-ethylurea

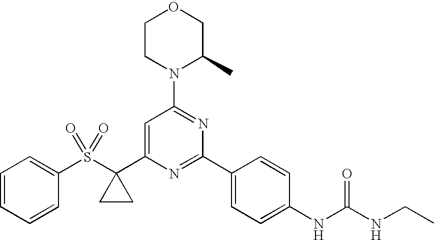

Triethylamine (0.064 mL, 0.46 mmol) was added to [2-(4-aminophenyl)-6-[1-(benzenesulfonyl)cyclopropyl]pyrimidin-4-yl]trifluoromethanesulfonate (0.104 g, 0.21 mmol), (R)-3-methylmorpholine (0.042 g, 0.42 mmol) in dioxane (5 mL) and the resulting solution stirred at RT overnight. Ethyl isocyanate (0.494 mL, 6.25 mmol) was added and the solution was allowed to stir at RT overnight. Methanol was added carefully and then all of the solvent was removed. The crude product was purified by preparative HPLC, eluting with decreasingly polar mixtures of water (containing 1% ammonia) and acetonitrile, to give the desired material as a tan solid (0.052 g). NMR Spectrum: ¹H NMR (400MHz, DMSO-d₆) 1.05-1.08 (3H, t), 1.16-1.18 (3H, d), 1.60-1.67 (2H, m), 1.88-1.92 (2H, m), 3.09-3.19 (3H, m), 3.42-3.49 (1H, td), 3.59-3.62 (1H, dd), 3.73-3.76 (1H, d), 3.94-3.97 (1H, dd), 4.08-4.12 (1H, d), 4.37(1H, bs), 6.13-6.16 (1H, t), 6.62 (1H, s), 7.37-7.39 (2H, d), 7.57-7.61 (2H, t), 7.70-7.74 (1H, tt), 7.78-7.85 (4H, m), 8.63 (1H, s).

LCMS Spectrum: m/z (ES+)(M+H)+=522; HPLC tR=2.32 min.

### [2-(4-Aminophenyl)-6-[1-(benzenesulfonyl)cyclopropyl]pyrimidin-4-yl]trifluoromethanesulfonate

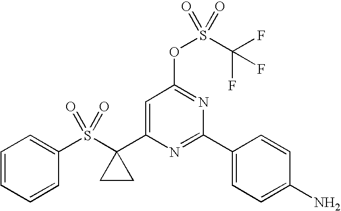

2-(4-Aminophenyl)-6-[1-(benzenesulfonyl)cyclopropyl]pyrimidin-4-ol (4.0 g, 10.89 mmol), 1,1,1-trifluoro-N-phenyl-N-(trifluoromethylsulfonyl)methanesulfonamide (4.28 g, 11.98 mmol)were dissolved in DCM (75 mL), to this was added DBU (1.791 mL, 11.98 mmol) and the reaction was stirred at RT overnight. The solvent was evaporated to dryness and the gum was quenched with 1.0N citric acid (100 mL) and extracted with diethyl ether (3×100 mL). The combined organics were dried over MgSO₄, filtered and evaporated to afford an orange solid. The solid was passed through a silica plug, eluting with diethyl ether, to give a yellow solid. This solid was dissolved in a minimum amount hot diethyl ether, to which iso-hexane was added and the system was stirred to afford the desired material as a yellow solid (1.5 g).

NMR Spectrum: ¹H NMR (400MHz, DMSO-d₆) 1.79 (2H, q), 2.02 (2H, q), 5.43 (2H, s), 6.62 (2H, d), 7.43 (1H, s), 7.59 (2H, t), 7.68 (2H, d), 7.72 (1H, t), 7.80 (2H, d).

LCMS Spectrum: m/z (ES+)(M+H)+=500; HPLC tR=2.96 min

### 2-(4-Aminophenyl)-6-[1-(benzenesulfonyl)cyclopropyl]pyrimidin-4-ol

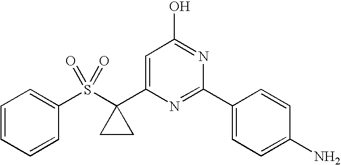

Ethyl 3-[1-(benzenesulfonyl)cyclopropyl]-3-oxopropanoate (5.4 g, 15.12 mmol), 4-aminobenzamidine dihydrochloride (3.78 g, 18.15 mmol) and potassium carbonate (3.83 mL, 63.52 mmol) were added to methanol (150 mL) and heated at reflux overnight. The solvent was evaporated to dryness and the remaining solid was acidified with 1.0N citric acid. The solid was filtered and then triturated with hot acetonitrile to give the desired material as a yellow solid (4.0 g). NMR Spectrum: ¹H NMR (400MHz, DMSO-d₆) 1.58 (2H, q), 1.86 (2H, q), 5.86 (2H, s), 6.21 (1H, s), 6.50 (2H, d), 7.51 (2H, d), 7.60 (2H, t), 7.72 (1H, t), 7.82 (2H, d), 12.11 (1H, s).

LCMS Spectrum: m/z (ES+)(M+H)+=368; HPLC tR=1.23 min

### Ethyl 3-[1-(benzenesulfonyl)cyclopropyl]-3-oxopropanoate

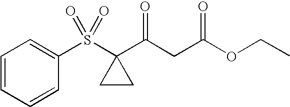

Triethylamine (20.70 mL, 148.51 mmol) and magnesium chloride (9.28 g, 97.46 mmol) were added to a stirred solution of potassium 3-ethoxy-3-oxopropanoate (15.80 g, 92.82 mmol) in acetonitrile (150 mL). The reaction was stirred for 2 hours. 1-(Benzenesulfonyl)cyclopropane-1-carboxylic acid (10.5 g, 46.41 mmol) and 1,1′-carbonyldiimidazole (9.03 g, 55.69 mmol) were dissolved in acetonitrile (30 mL) and stirred for 2 hours. This was then added to the initial reaction and the system was stirred over the weekend at RT. 2M hydrochloric acid (150 mL) was added and the mixture extracted with diethyl ether (3×50 mL), the organics separated and evaporated to afford an orange oil. This oil was washed with a saturated solution of sodium hydrogen carbonate (100 mL) and extracted with diethyl ether (3×100 mL). The organics were dried over MgSO₄, filtered and evaporated to afford a yellow gum. This was passed through a plug of silica, eluting with DCM, to afford a colourless gum. The crude product was further purified by flash silica chromatography, eluting with DCM, to give the desired material as a colourless gum (5.6 g).

NMR Spectrum: ¹H NMR (400MHz, CDCl₃) δ 1.23 (3H, t), 1.74 (2H, q), 1.99 (2H, q), 3.77 (2H, s), 4.12 (2H, q), 7.57 (2H, t), 7.67 (1H, t), 7.92 (2H, d).

### 1-(Benzenesulfonyl)cyclopropane-1-carboxylic acid

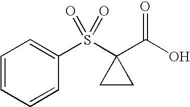

Methyl 1-(benzenesulfonyl)cyclopropane-1-carboxylate (11 g, 45.78 mmol) was added to ethanol (50 mL) and water (50 mL), to this was added sodium hydroxide (1.904 mL, 48.07 mmol) and the reaction was stirred for 1 hour. The ethanol was carefully evaporated and the reaction mixture was extracted with diethyl ether (1×100 mL). The aqueous layer was quenched with 2M hydrochloric acid (50 mL), extracted with diethyl ether (3×100 mL) and the combined organics dried over MgSO4, filtered and evaporated to afford a white solid. The 20 crude solid was triturated with ethyl acetate to give the desired material as a white solid (10.5 g).

NMR Spectrum: ¹H NMR (400 MHz, DMSO-d₆) δ 1.63 (2H, q), 1.85 (2H, q), 7.63 (2H, t), 7.73 (1H, t), 7.97 (2H, d).

### Methyl 1-(benzenesulfonyl)cyclopropane-1-carboxylate

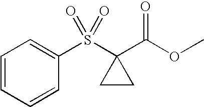

Methyl 2-(phenylsulfonyl)acetate (15 g, 70.02 mmol), benzyltriethylammonium chloride (4.77 g, 21.00 mmol), Potassium carbonate (29.0 g, 210.05 mmol) and 1,2-dibromoethane (12.07 mL, 140.03 mmol) were added to toluene (200 mL) and heated at 100° C. over the weekend. The reaction was filtered and solvent evaporated to afford a viscous gum. This reaction mixture was quenched with water (100 mL) and extracted with diethyl ether (3×100 mL). The organics were dried over MgSO₄, filtered and evaporated to afford an orange liquid. This liquid was passed through a plug of silica, eluting with diethyl ether, to afford an orange gum. The crude gum was triturated with ethanol to give the desired material as a white solid (11.0 g).

NMR Spectrum: ¹H NMR (400 MHz, CDCl₃) δ 11.70 (2H, q), 2.00 (2H, q), 3.63 (3H, s), 7.55 (2H, t), 7.64 (1H, t), 8.00 (2H, d).

LCMS Spectrum: m/z (ES+)(M+H)+=241; HPLC tR=1.84 min

### EXAMPLE 70

### 1-[4-[4-[1-(Benzenesulfonyl)cyclopropyl]-6-[(3S)-3-ethylmorpholin-4-yl]pyrimidin-2-yl]phenyl]-3-ethylurea

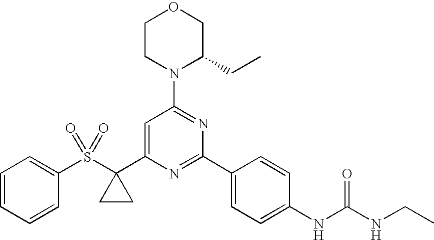

Triethylamine (0.062 mL, 0.45 mmol) was added to [2-(4-aminophenyl)-6-[1-(benzenesulfonyl)cyclopropyl]pyrimidin-4-yl]trifluoromethanesulfonate (0.102 g, 0.20 mmol), (S)-3-ethylmorpholine (0.047 g, 0.41 mmol) in dioxane (5 mL) and the resulting solution stirred at 50° C. overnight. Ethyl isocyanate (0.564 mL, 7.12 mmol) was added and the solution allowed to stir at RT overnight. Methanol was added and then the solvent was removed. The crude product was purified by preparative HPLC, to give the desired material as a white solid (0.042 g).

NMR Spectrum: ¹H NMR (400 MHz, DMSO-d₆) δ 0.83-0.86(3H, t), 1.05-1.08(3H, t), 1.56-1.67(4H, m), 1.71-1.80(1H, m), 1.88-1.91(2H, m), 3.09-3.16(3H, m), 3.40-3.47(1H, td), 3.50-3.54(1H, dd), 3.84-3.87(1H, d), 3.90-3.94(1H, dd), 4.18(1H, bs), 6.12-6.15(1H, t), 6.61(1H, s), 7.37-7.40(2H, d), 7.57-7.61(2H, t), 7.69-7.74(1H, tt), 7.77-7.80(2H, dd), 7.84-7.86(2H, d), 8.61(1H, s).

LCMS Spectrum: m/z (ES+)(M+H)+=536; HPLC tR=2.43 min.

The preparation of [2-(4-aminophenyl)-6-[1-(benzenesulfonyl)cyclopropyl]pyrimidin-4-yl]trifluoromethanesulfonate was described earlier.

### EXAMPLE 71

### 1-[4-[4-[1-(Benzenesulfonyl)cyclopropyl]-6-[3-(hydroxymethyl)morpholin-4-yl]pyrimidin-2-yl]phenyl]-3-ethylurea

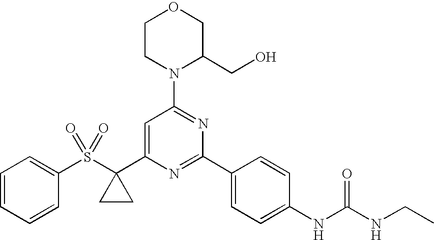

Triethylamine (0.1 12 mL, 0.80 mmol) was added to [2-(4-aminophenyl)-6-[1-(benzenesulfonyl)cyclopropyl]pyrimidin-4-yl]trifluoromethanesulfonate (0.100 g, 0.20 mmol), morpholin-3-ylmethanol hydrochloride (0.062 g, 0.40 mmol) in dioxane (5 mL) and the resulting solution stirred at 50° C. overnight. Ethyl isocyanate (0.555 mL, 7.01 mmol) was added and the solution allowed to stir at RT overnight. The solvent was removed and the sludge was taken up in methanol. 30% Sodium methoxide in methanol solution was added and the reaction was allowed to stir overnight. Additional 30% sodium methoxide in methanol solution was added and the reaction refluxed overnight. Approximately 80% of the methanol was removed and the solution was quenched with saturated ammonium chloride solution. The mixture was extracted with DCM, the organics dried over MgSO₄, filtered and evaporated. The crude product was purified by preparative HPLC, to give the desired material as a cream solid (0.019 g).

NMR Spectrum: ¹H NMR (400 MHz, DMSO-d₆) δ 1.05-1.08 (3H, t), 1.58-1.66 (2H, m), 1.86-1.92 (2H, m), 3.07-3.16 (3H, m), 3.42-3.53 (3H, m), 3.69-3.73 (1H, m), 3.92-3.95 1H, dd), 4.05-4.08 (1H, d), 4.18 (1H, bs), 4.93 (1H, bs), 6.14-6.17 (1H, t), 6.68 (1H, s), 7.36 (2H, d), 7.57-7.61 (2H, t), 7.70-7.74 (1H, tt), 7.79-7.84 (4H, m), 8.64 (1H, s) (1 peak under water or solvent peak).

LCMS Spectrum: m/z (ES+)(M+H)+=538; HPLC tR=1.95 min.

### Morpholin-3-ylmethanol

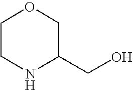

(4-Benzylmorpholin-3-yl)methanol (0.473 g, 2.28 mmol) and palladium (5% on carbon, 50% wet) (0.094 g, 0.02 mmol) in ethanol (50 mL) were stirred under an atmosphere of hydrogen at 5 bar and 25° C. for 18 hours. The mixture was filtered and then hydrochloric acid (0.628 mL, 2.51 mmol) added. The reaction was stirred overnight at RT and then the solvent removed to give the desired material (as a hydrochloride salt) as an orangey brown gum (257 mg).

NMR Spectrum: ¹H NMR (400 MHz, CDCl₃) δ 1.43-1.46(1H, t), 3.14-3.67(3H, m), 3.81-4.18(4H, m), 4.62(1H, bs), 9.32(bs), 9.60(bs), 10.43(bs).

The preparation of [2-(4-aminophenyl)-6-[1-(benzenesulfonyl)cyclopropyl]pyrimidin-4-yl]trifluoromethanesulfonate was described earlier.

### EXAMPLE 72

### 1-[4-[4-[1-(Benzenesulfonyl)cyclopropyl]-6-[(3S,5S)-3,5-dimethylmorpholin-4-yl]pyrimidin-2-yl]phenyl]-3-ethylurea

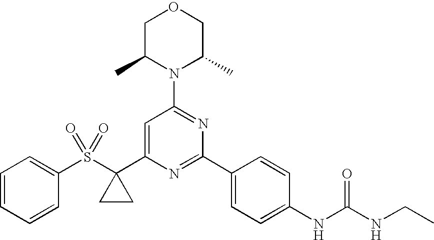

DIPEA (0.141 mL, 0.81 mmol) was added to [2-(4-aminophenyl)-6-[1-(benzenesulfonyl)cyclopropyl]pyrimidin-4-yl]trifluoromethanesulfonate (0.101 g, 0.20 mmol) and (3S,5S)-3,5-dimethylmorpholine (hydrochloride salt) (0.061 g, 0.40 mmol) in dioxane (5 mL) under nitrogen. The resulting solution was stirred at 70° C. overnight then at 90° C. for several hours. Additional (3S,5S)-3,5-dimethylmorpholine (hydrochloride salt) was added and the reaction was allowed to stir at 90° C. overnight. Ethyl isocyanate (0.320 mL, 4.05 mmol) was added and the reaction allowed to stir at RT over the weekend. Methanol was added and then the solvent was removed. The crude product was purified by preparative HPLC, to give the desired material as a cream solid (0.029 g).

NMR Spectrum: ¹H NMR (400 MHz, DMSO-d₆) δ 1.05-1.08 (3H, t), 1.27-1.29(6H, d), 1.71 (2H, m), 1.88-1.95 (2H, m), 3.09-3.16 (2H, m), 3.67-3.70 (2H, m), 4.10-4.16 (4H, m), 6.14-6.16 (1H, t), 6.60 (1H, s), 7.39-7.41 (2H, d), 7.57-7.61 (2H, t), 7.69-7.73 (1H, tt), 7.78-7.81 (2H, dd), 7.86-7.89 (2H, d), 8.62 (1H, s).

LCMS Spectrum: m/z (ES+)(M+H)+=536; HPLC tR=2.41 min.

The preparation of [2-(4-aminophenyl)-6-[1-(benzenesulfonyl)cyclopropyl]pyrimidin-4-yl]trifluoromethanesulfonate was described earlier.

### (3S,5S)-3,5-Dimethylmorpholine

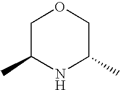

Hydrogen chloride (4M solution in dioxane, 30.1 mL, 120.25 mmol) was added to tert-butyl (3S,5S)-3,5-dimethylmorpholine-4-carboxylate (5.23 g, 24.29 mmol) in dioxane (50 mL) and the resulting solution stirred at RT overnight. The solvent was removed and the solid was triturated with diethyl ether to give the desired material (as the hydrochloride salt) as a white solid (3.22 g).

NMR Spectrum: ¹H NMR (400 MHz, CDCl₃) δ 1.50-1.51(6H, d), 3.56-3.67 (4H, m), 3.97-4.00 (2H, dd), 9.96 (2H, bs).

### tert-Butyl (3S,5S)-3,5-dimethylmorpholine-4-carboxylate and tert-butyl (3S,5R)-3,5-dimethylmorpholine-4-carboxylate

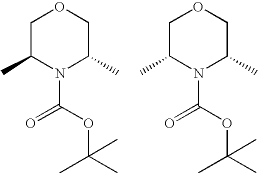

(3S)-3,5-Dimethylmorpholine (13.73 g, 90.55 mmol) was dissolved in a solution of sodium hydroxide (3.91 mL, 208.26 mmol) in water (100 mL) and di-tert-butyl dicarbonate (22.88 mL, 99.60 mmol) added portionwise. The resulting solution was stirred at RT overnight then extracted with diethyl ether and the organics dried over MgSO₄, filtered and evaporated to give a clear liquid. The crude material was purified and the diastereomers separated using silica chromatography, eluting with 0-10% ethyl acetate in isohexane, to give tert-butyl (3S,5S)-3,5-dimethylmorpholine-4-carboxylate (first product to elut) as a colourless liquid (5.93 g) and tert-butyl (3S,5R)-3,5-dimethylmorpholine-4-carboxylate (second product to elut) as a colourless liquid (5.03 g).

### tert-butyl (3S,5S)-3,5-dimethylmorpholine-4-carboxylate

NMR Spectrum: ¹H NMR (400 MHz, CDCl₃) δ 1.28-1.29(6H, d), 1.47(9H, s), 3.43-3.48 (2H, m), 3.77-3.84 (4H, m).

### tert-butyl (3S,5R)-3,5-dimethylmorpholine-4-carboxylate

NMR Spectrum: ¹H NMR (400 MHz, CDCl₃) δ 1.30-1.31(6H, d), 1.47(9H, s), 3.52-3.56(2H, dd), 3.68-3.71(2H, d), 3.90-3.96(2H, m).

### (3S)-3 5-Dimethylmorpholine

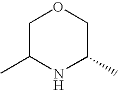

2-[[(2S)-1-Hydroxypropan-2-yl]amino]propan-1-ol (14.79 g, 111.01 mmol) was cooled to 0° C. with stirring and concentrated sulfuric acid (19.85 g, 202.39 mmol) added. The mixture was heated to 180° C. for 5 hours. Potassium hydroxide (23.95 g, 426.87 mmol) in water (120 mL) was added slowly then the mixture filtered to leave a dark black aqueous solution. This solution was distilled (distillate came off at 98° C.) and the distillate acidified with 2M hydrochloric acid. The water was removed to give the desired material (as the hydrochloride salt) as a white solid (13.73 g). The material was used in the following step without further purification.

NMR Spectrum: ¹H NMR (400 MHz, CDCl₃) δ 1.44-1.46 (3H, d), 1.48-1.50 (3H, d), 3.31-3.34 (1H, m), 3.56-3.59 (1H, m), 3.62-3.72 (2H, m), 3.84-3.88 (1H, m), 3.96-3.99 (1H, dd), 9.69-10.12 (2H, bt).

### 2-[[(2S)-1-Hydroxypropan-2-yl]amino]propan-1-ol

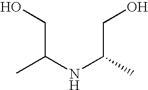

(S)-2-Aminopropan-1-ol (9 g, 119.82 mmol), platinum(IV) oxide (0.052 g, 0.23 mmol) and 1-hydroxypropan-2-one (11.54 g, 155.77 mmol) in methanol (100 mL) were stirred for 1 hour and then placed under an atmosphere of hydrogen at 1 bar and 25° C. for 3 hours. The solution was filtered and evaporated to give a crude product that was purified by distillation at (0.55 mBar, 92° C.) to give the desired material as a yellow oil (10.71 g).

NMR Spectrum: ¹H NMR (400 MHz, CDCl₃) δ 1.01-1.03 (3H, d), 1.07-1.09 (3H, d), 2.56 (1H, bs), 2.83-2.94 (2H, m), 3.27-3.32 (2H, m), 3.55-3.61 (2H, m).

### EXAMPLE 73

### 1-[4-[4-[1-(Benzenesulfonyl)cyclopropyl]-6-[(3R,5S)-3,5-dimethylmorpholin-4-yl]pyrimidin-2-yl]phenyl]-3-ethylurea

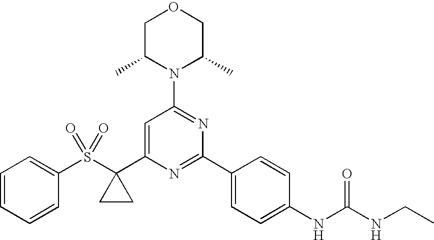

DIPEA (0.140 mL, 0.81 mmol) was added to [2-(4-aminophenyl)-6-[1-(benzenesulfonyl)cyclopropyl]pyrimidin-4-yl]trifluoromethanesulfonate (0.101 g, 0.20 mmol), (3S,5R)-3,5-dimethylmorpholine (as the hydrochloride salt) (0.052 g, 0.34 mmol) in dioxane (5 mL) under nitrogen. The reaction was heated to 90° C. overnight. The reaction cooled and the solvent was removed. The residue was taken up in dioxane and ethyl isocyanate (0.319 mL, 4.03 mmol) added. The reaction was allowed to stir over the weekend. Methanol was added and the solvent was removed. The crude product was purified by preparative HPLC, to give the desired material as a white solid (9.00 mg).

NMR Spectrum: ¹H NMR (400 MHz, DMSO-d₆) δ 1.05-1.09 (3H, t), 1.17-1.20(6H, m), 1.62-1.68 (2H, m), 1.89-1.92 (2H, m), 3.09-3.14 (2H, m), 3.43-3.48 (2H, m), 3.86-3.90 (1H, dd), 3.94-4.16 (1H, dd), 4.12-4.16 (1H, m), 4.29-4.31 (1H, m), 6.12-6.15 (1H, t), 6.60 (1H, s), 7.37-7.40 (2H, d), 7.57-7.61 (2H, t), 7.70-7.74 (1H, tt), 7.79-7.86 (4H, m), 8.61 (1H, s).

LCMS Spectrum: m/z (ES+)(M+H)+=536; HPLC tR=2.38 min.

The preparation of [2-(4-aminophenyl)-6-[1-(benzenesulfonyl)cyclopropyl]pyrimidin-4-yl]trifluoromethanesulfonate was described earlier.

### (3S,5R)-3 5-Dimethylmorpholine

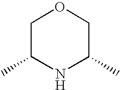

Hydrogen chloride (4M solution in dioxane, 9.98 mL, 39.92 mmol) was added to tert-butyl (3S,5R)-3,5-dimethylmorpholine-4-carboxylate (1.910 g, 8.87 mmol) in dioxane (15 mL) and the resulting solution stirred at RT overnight. The solvent was removed and the solid was triturated with diethyl ether to give the desired material (as the hydrochloride salt) as a white solid (0.960 g).

NMR Spectrum:¹H NMR (400 MHz, CDCl₃) δ 1.47-1.48(6H, d), 3.31-3.40 (2H, m), 3.68-3.74 (2H, t), 3.86-3.90 (2H, dd), 9.77 (1H, bs), 10.22 (1H, bs).

The preparation of tert-butyl (3S,5R)-3,5-dimethylmorpholine-4-carboxylate was described earlier.

### EXAMPLE 74

### 1-[4-[4-[1-(Benzenesulfonyl)cyclopropyl]-6-[(3S)-3-ethylmorpholin-4-yl]pyrimidin-2-yl]phenyl]-3-methylurea

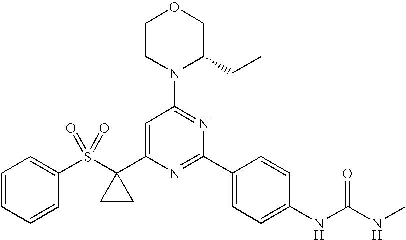

Phenyl N-[4-[4-[1-(benzenesulfonyl)cyclopropyl]-6-[(3S)-3-ethylmorpholin-4-yl]pyrimidin-2-yl]phenyl]carbamate (0.1 g, 0.17 mmol), triethylamine (0.072 mL, 0.51 mmol) and methylamine (0.51 mmol) were dissolved in dioxane (10 mL) and heated at 50° C. overnight. The reaction was evaporated to dryness and was purified by preparative HPLC, eluting with decreasingly polar mixtures of water (containing 1% ammonia) and acetonitrile, to give the desired material as a white solid.

NMR Spectrum: ¹H NMR (400.132 MHz, DMSO-d₆) δ 0.84 (3H, t), 1.65-1.56 (3H, m), 1.80-1.71 (1H, m), 1.91-1.87 (2H, m), 2.66 (3H, d), 3.12 (1H, ddd), 3.43 (1H, ddd), 3.51 (1H, dd), 3.85 (1H, d), 3.91 (1H, dd), 4.24-4.12 (2H, m), 6.04 (1H, q), 6.61 (1H, s), 7.39 (2H, d), 7.58 (2H, t), 7.71 (1H, t), 7.78 (2H, d), 7.84 (2H, d), 8.69 (1H, s).

LCMS Spectrum: m/z (ES+) (M+H)+=522; HPLC tR=2.31 min;

The following compounds were prepared in an analogous fashion from phenyl N-[4-[4-[1-(benzenesulfonyl)cyclopropyl]-6-[(3S)-3-ethylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 74a | 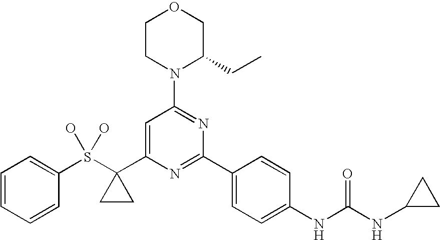 | 1-[4-[4-[1-(benzenesulfonyl)cyclopropyl]-6-[(3S)-3-ethylmorpholin-4-yl]pyrimidin-2-yl]phenyl]-3-cyclopropylurea | 548 | 2.46 |
| 74b | 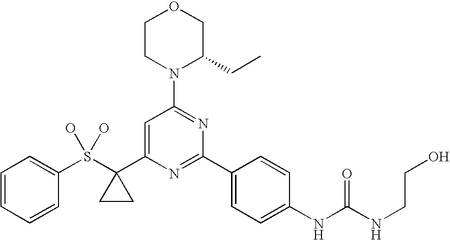 | 1-[4-[4-[1-(benzenesulfonyl)cyclopropyl]-6-[(3S)-3-ethylmorpholin-4-yl]pyrimidin-2-yl]phenyl]-3-(2-hydroxyethyl)urea | 552 | 2.11 |
| 74c | 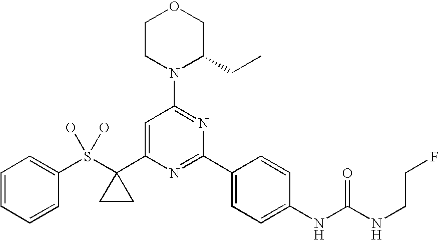 | 1-[4-[4-[1-(benzenesulfonyl)cyclopropyl]-6-[(3S)-3-ethylmorpholin-4-yl]pyrimidin-2-yl]phenyl]-3-(2-fluoroethyl)urea | 554 | 1.95 |
| 74d | 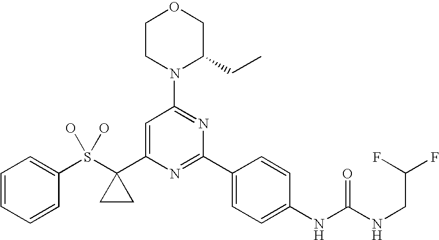 | 1-[4-[4-[1-(benzenesulfonyl)cyclopropyl]-6-[(3S)-3-ethylmorpholin-4-yl]pyrimidin-2-yl]phenyl]-3-(2,2-difluoroethyl)urea | 572 | 2.45 |
| 74e | 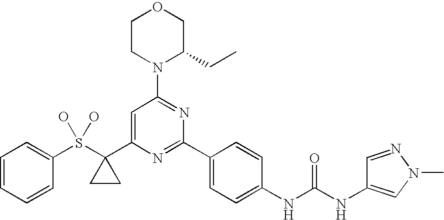 | 1-[4-[4-[1-(benzenesulfonyl)cyclopropyl]-6-[(3S)-3-ethylmorpholin-4-yl]pyrimidin-2-yl]phenyl]-3-(1-methylpyrazol-4-yl)urea | 588 | 2.33 |
| 74f | 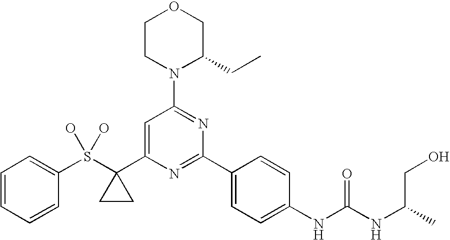 | 1-[4-[4-[1-(benzenesulfonyl)cyclopropyl]-6-[(3S)-3-ethylmorpholin-4-yl]pyrimidin-2-yl]phenyl]-3-[(2S)-1-hydroxypropan-2-yl]urea | 566 | 2.21 |
| 74g | 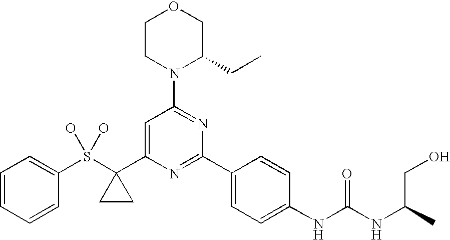 | 1-[4-[4-[1-(benzenesulfonyl)cyclopropyl]-6-[(3S)-3-ethylmorpholin-4-yl]pyrimidin-2-yl]phenyl]-3-[(2R)-1-hydroxypropan-2-yl]urea | 566 | 2.21 |
| 74h | 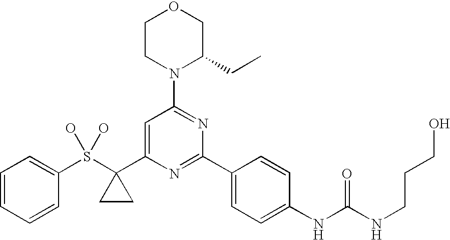 | 1-[4-[4-[1-(benzenesulfonyl)cyclopropyl]-6-[(3S)-3-ethylmorpholin-4-yl]pyrimidin-2-yl]phenyl]-3-(3-hydroxypropyl)urea | 578 | 2.28 |
| 74i | 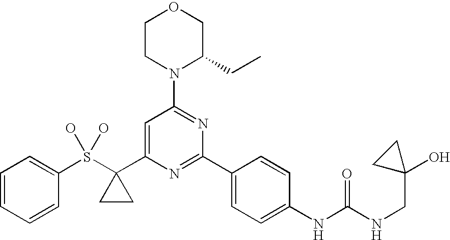 | 1-[4-[4-[1-(benzenesulfonyl)cyclopropyl]-6-[(3S)-3-ethylmorpholin-4-yl]pyrimidin-2-yl]phenyl]-3-[(1-hydroxycyclopropyl)methyl]urea | 578 | 2.29 |

### EXAMPLE 74a

¹H NMR (400.132 MHz, DMSO-d₆) δ 0.43-0.39 (2H, m), 0.67-0.62 (2H, m), 0.84 (3H, t), 1.64-1.58 (3H, m), 1.80-1.73 (1H, m), 1.92-1.85 (2H, m), 2.57-2.52 (1H, m), 3.12 (1H, ddd), 3.43 (1H, ddd), 3.51 (1H, dd), 3.85 (1H, d), 3.91 (1H, dd), 4.28-4.09 (2H, m), 6.40 (1H, s), 6.61 (1H, s), 7.39 (2H, d), 7.58 (2H, t), 7.71 (1H, t), 7.78 (2H, d), 7.85 (2H, d), 8.49 (1H, s);

### EXAMPLE 74b

¹H NMR (400.132 MHz, DMSO-d₆) δ 0.84 (3H, t), 1.64-1.58 (3H, m), 1.80-1.80-1.73 (1H, m), 1.91-1.86 (2H, m), 3.19-3.09 (3H, m), 3.48-3.40 (3H, m), 3.51 (1H, dd), 3.85 (1H, d), 3.91 (1H, dd), 4.26-4.08 (2H, m), 4.72 (1H, t), 6.23 (1H, t), 6.61 (1H, s), 7.38 (2H, d), 7.58 (2H, t), 7.71 (1H, t), 7.78 (2H, d), 7.85 (2H, d), 8.75 (1H, s);

### EXAMPLE 74c

¹H NMR (400.132 MHz, DMSO-d₆) δ 0.84 (3H, t), 1.64-1.58 (3H, m), 1.80-1.71 (1H, m), 1.91-1.87 (2H, m), 3.12 (1H, ddd), 3.47-3.36 (3H, m), 3.51 (1H, dd), 3.85 (1H, d), 3.91 (1H, dd), 4.28-4.09 (2H, m), 4.47 (2H, dt), 6.42 (1H, t), 6.62 (1H, s), 7.39 (2H, d), 7.58 (2H, t), 7.71 (1H, t), 7.78 (2H, d), 7.86 (2H, d), 8.77 (1H, s);

### EXAMPLE 74d

¹H NMR (400.132 MHz, DMSO-d₆) δ 0.84 (3H, t), 1.65-1.58 (3H, m), 1.80-1.73 (1H, m), 1.91-1.87 (2H, m), 3.12 (1H, ddd), 3.43 (1H, ddd), 3.59-3.50 (3H, m), 3.85 (1H, d), 3.92 (1H, dd), 4.27-4.12 (2H, m), 6.07 (1H, tt), 6.51 (1H, t), 6.62 (1H, s), 7.40 (2H, d), 7.58 (2H, t), 7.71 (1H, t), 7.78 (2H, d), 7.87 (2H, d), 8.87 (1H, s);

### EXAMPLE 74e

¹H NMR (400.132 MHz, DMSO-d₆) δ 0.85 (3H, t), 1.68-1.59 (3H, m), 1.80-1.73 (1H, m), 1.92-1.87 (2H, m), 3.12 (1H, ddd), 3.43 (1H, ddd), 3.52 (1H, dd), 3.79 (3H, s), 3.86 (1H, d), 3.92 (1H, dd), 4.30-4.18 (2H, m), 6.62 (1H, s), 7.38 (1H, s), 7.43 (2H, d), 7.59 (2H, t), 7.72 (1H, t), 7.76 (1H, s), 7.78 (2H, d), 7.88 (2H, d), 8.36 (1H, s), 8.78 (1H, s);

### EXAMPLE 74f

¹H NMR (400.132 MHz, DMSO-d₆) δ 0.84 (3H, t), 1.08 (3H, d), 1.65-1.58 (3H, m), 1.82-1.73 (1H, m), 1.90-1.88 (2H, m), 3.12 (1H, ddd), 3.39-3.33 (2H, m), 3.43 (1H, ddd), 3.51 (1H, dd), 3.73-3.67 (1H, m), 3.85 (1H, d), 3.91 (1H, dd), 4.25-4.13 (2H, m), 4.78 (1H, t), 6.07 (1H, d), 6.61 (1H, s), 7.36 (2H, d), 7.58 (2H, t), 7.71 (1H, t), 7.78 (2H, d), 7.85 (2H, d), 8.66 (1H, s);

### EXAMPLE 74g

¹H NMR (400.132 MHz, DMSO-d₆) δ 0.84 (3H, t), 1.08 (3H, d), 1.67-1.58 (3H, m), 1.80-1.73 (1H, m), 1.90-1.88 (2H, m), 3.12 (1H, ddd), 3.40-3.32 (2H, m), 3.44 (1H, ddd), 3.51 (1H, dd), 3.73-3.67 (1H, m), 3.85 (1H, d), 3.91 (1H, dd), 4.27-4.08 (2H, m), 4.78 (1H, t), 6.07 (1H, d), 6.61 (1H, s), 7.36 (2H, d), 7.58 (2H, t), 7.71 (1H, t), 7.78 (2H, d), 7.85 (2H, d), 8.66 (1H, s);

### EXAMPLE 74h

¹H NMR (400.132 MHz, DMSO-d₆) δ 0.84 (3H, t), 1.64-1.56 (5H, m), 1.82-1.69 (1H, m), 1.91-1.87 (2H, m), 3.19-3.08 (3H, m), 3.53-3.40 (4H, m), 3.85 (1H, d), 3.91 (1H, dd), 4.26-4.08 (2H, m), 4.47 (1H, t), 6.18 (1H, t), 6.61 (1H, s), 7.38 (2H, t), 7.71 (1H, t), 7.78 (2H, d), 7.85 (2H, d), 8.66 (1H, s);

### EXAMPLE 74i

¹H NMR (400.132 MHz, DMSO-d₆) δ 0.53-0.48 (2H, m), 0.59-0.56 (2H, m), 0.84 (3H, t), 1.64-1.58 (3H, m), 1.80-1.73 (1H, m), 1.92-1.88 (2H, m), 3.12 (1H, ddd), 3.21 (2H, d), 3.46-3.38 (1H, m), 3.51 (1H, dd), 3.85 (1H, d), 3.91 (1H, dd), 4.27-4.05 (2H, m), 6.30 (1H, t), 6.61 (1H, s), 7.38 (2H, d), 7.58 (2H, t), 7.71 (1H, t), 7.78 (2H, d), 7.85 (2H, d), 8.76 (1H, s), hydroxy missing

The preparation of phenyl N-[4-[4-[1-(benzenesulfonyl)cyclopropyl]-6-[(3S)-3-ethylmorpholin-4-yl]pyrimidin-2-yl]phenyl]carbamate is described below.

### Phenyl N-[4-[4-[1-(benzenesulfonyl)cyclopropyl]-6-[(3S)-3-ethylmorpholin-4-yl]pyrimidin-2-yl]phenylcarbamate

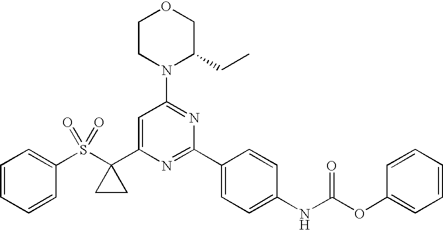

4-[4-[1-(Benzenesulfonyl)cyclopropyl]-6-[(3S)-3-ethylmorpholin-4-yl]pyrimidin-2-yl]aniline (as the hydrochloride salt) (1.418 g, 2.83 mmol) and sodium bicarbonate (2.377 g, 28.30 mmol) were added to DCM (60 mL) and stirred for 10 minutes. Phenyl chloroformate (0.462 mL, 3.68 mmol) was added slowly and the reaction stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. The crude product was purified by flash silica chromatography, elution gradient 30 to 60% ethyl acetate in isohexane, to give the desired material as a yellow solid (1.22 g).

NMR Spectrum: ¹H NMR (400.132 MHz, DMSO-d₆) δ 0.84 (3H, t), 1.68-1.59 (3H, m), 1.80-1.73 (1H, m), 1.92-1.89 (2H, m), 3.13 (1H, ddd), 3.43 (1H, ddd), 3.52 (1H, dd), 3.85 (1H, d), 3.92 (1H, dd), 4.18 (2H, s), 6.66 (1H, s), 7.29-7.24 (3H, m), 7.45 (2H, t), 7.54 (2H, d), 7.58 (2H, t), 7.71 (1H, t), 7.79 (2H, d), 7.95 (2H, d), 10.39 (1H, s);

LCMS Spectrum: m/z (ES+) (M+H)+=585; HPLC tR=3.12 min;

### 4-[4-[1-(Benzenesulfonyl)cyclopropyl]-6-[(3S)-3-ethylmorpholin-4-yl]pyrimidin-2-yl]aniline

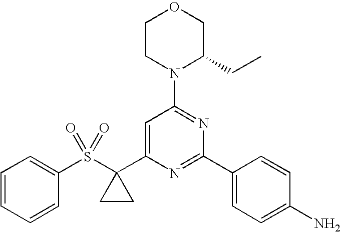

tert-Butyl N-[4-[4-[1-(benzenesulfonyl)cyclopropyl]-6-[(3S)-3-ethylmorpholin-4-yl]pyrimidin-2-yl]phenyl]carbamate (1.6 g, 2.83 mmol) was added to 6.0 N hydrogen chloride in propan-2-ol (20 mL) and stirred for 2 hours at RT. The crude solution was triturated with diethyl ether to give the desired material (as the hydrochloride salt) as a yellow solid (1.40 g).

NMR Spectrum: ¹H NMR (400.132 MHz, DMSO-d₆) δ 0.81-0.71 (3H, m), 1.78-1.60 (5H, m), 1.96-1.87 (2H, m), 3.20 (1H, ddd), 3.41 (1H, ddd), 3.50 (1H, dd), 3.84 (1H, d), 3.93 (1H, dd), 6.56 (1H, s), 7.01 (2H, s), 7.61 (2H, t), 7.79-7.75 (4H, m), 7.95 (2H, d);

LCMS Spectrum: m/z (ES+) (M+H)+=465; HPLC tR=2.54 min;

### tert-Butyl N-[4-[4-[1-(benzenesulfonyl)cyclopropyl]-6-[(3S)-3-ethylmorpholin-4-yl]pyrimidin-2-yl]phenyl]carbamate

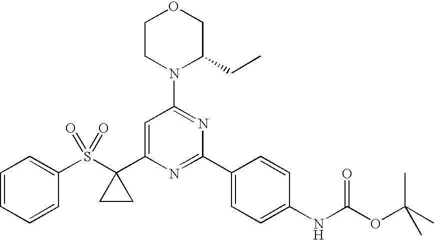

Sodium hydride (0.724 g, 15.07 mmol) was added rapidly to tert-butyl N-[4-[4-(benzenesulfonylmethyl)-6-[(3S)-3-ethylmorpholin-4-yl]pyrimidin-2-yl]phenyl]carbamate (2.03 g, 3.77 mmol) in DMF (30 mL) and the mixture stirred at RT for 10 minutes before the slow addition 1,2-dibromoethane (1.299 mL, 15.07 mmol) in DMF (30 mL). The resulting suspension was stirred at RT for 1 hour. Additional sodium hydride (0.36 g, 7.53 mmol) and 1,2 dibromoethane (0.65 mL, 7.53 mmol) were rapidly added and the reaction was stirred for a further 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to afford yellow foam. This was dissolved in 40% ethyl acetate in isohexane and stirred resulting in the desired material precipitating out as a white solid (1.65 g).

NMR Spectrum: ¹H NMR (400.132 MHz, DMSO-d₆) δ 0.84 (3H, t), 1.49 (9H, s), 1.65-1.58 (3H, m), 1.80-1.73 (1H, m), 1.90-1.87 (2H, m), 3.12 (1H, ddd), 3.43 (1H, ddd), 3.51 (1H, dd), 3.85 (1H, d), 3.91 (1H, dd), 4.18 (2H, s), 6.63 (1H, s), 7.45 (2H, d), 7.58 (2H, t), 7.71 (1H, t), 7.78 (2H, d), 7.87 (2H, d), 9.49 (1H, s);

LCMS Spectrum: m/z (ES+) (M+H)+=565; HPLC tR=3.23 min;

### tert-Butyl N-[4-[4-(benzenesulfonylmethyl)-6-[ (3S)-3-ethylmorpholin-4-yl]pyrimidin-2-yl]phenyl]carbamate

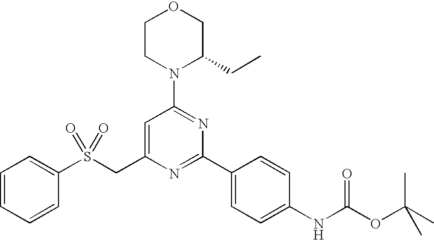

Sodium benzenesulfonate (0.626 g, 3.81 mmol) and tert-butyl N-[4-[4-[(3S)-3-ethylmorpholin-4-yl]-6-(iodomethyl)pyrimidin-2-yl]phenyl]carbamate (2.0 g, 3.81 mmol) were dissolved in DMF (25 mL) and stirred for 1 hour at RT. The solvent was evaporated to afford a yellow solid which was partitioned between aqueous sodium thiosulphate solution (50 mL) and DCM (75 mL). The organics were purified by flash silica chromatography, elution gradient 30 to 60% ethyl acetate in isohexane., to give the desired material as a white foam (1.99 g).

NMR Spectrum: ¹H NMR (400.132 MHz, DMSO-d₆) δ 0.86 (3H, t), 1.49 (9H, s), 1.66-1.57 (1H, m), 1.82-1.74 (1H, m), 3.14 (1H, ddd), 3.45 (1H, ddd), 3.53 (1H, dd), 3.87 (1H, d), 3.93 (1H, dd), 4.16 (2H, s), 4.69 (2H, s), 6.61 (1H, s), 7.45 (2H, d), 7.61 (2H, t), 7.74 (1H, t), 7.85-7.80 (4H, m), 9.49 (1H, s);

LCMS Spectrum: m/z (ES+) (M+H)+=539; HPLC tR=3.00 min;

### tert-Butyl N-[4-[4-[(3S)-3-ethylmorpholin-4-yl]-6-(iodomethyl)pyrimidin-2-yl]phenyl]carbamate

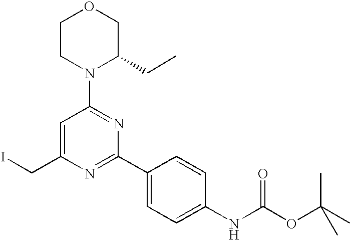

[6-[(3S)-3-Ethylmorpholin-4-yl]-2-[4-[(2-methylpropan-2-yl)oxycarbonylamino]phenyl]pyrimidin-4-yl]methyl methanesulfonate (10.34 g, 20.99 mmol) and lithium iodide (1.208 mL, 31.49 mmol) were added to dioxane (250 mL) and heated at 60° C. for 1 hour and then at RT overnight. The solvent was evaporated and the reaction mixture quenched with saturated ammonium chloride solution (100 mL) then extracted with DCM (3×75 mL). The organic extracts were flushed through a 2 inch silica plug, eluting 1o with ethyl acetate, to give a brown foam. This was dissolved in diethyl ether and isohexane carefully added until a cloudy solution was observed. Upon cooling to 0° C., the desired material precipitated out as a white solid and was isolated by filtration (9.80 g).

NMR Spectrum: ¹H NMR (400.132 MHz, DMSO-d₆) δ 0.88 (3H, t), 1.50 (9H, s), 1.72-1.63 (1H, m), 1.83-1.76 (1H, m), 3.17 (1H, ddd), 3.47 (1H, ddd), 3.55 (1H, dd), 3.87 (1H, d), 3.93 (1H, dd), 4.29 (2H, s), 4.38 (2H, s), 6.81 (1H, s), 7.56 (2H, d), 8.22 (2H, d), 9.53 (1H, s);

LCMS Spectrum: m/z (ES+) (M+H)+=525; HPLC tR=3.17 min;

### [6-[(3S)-3-Ethylmorpholin-4-yl]-2-[4-[(2-methylpropan-2-yl)oxycarbonylamino]phenyl]pyrimidin-4-yl]methyl methanesulfonate

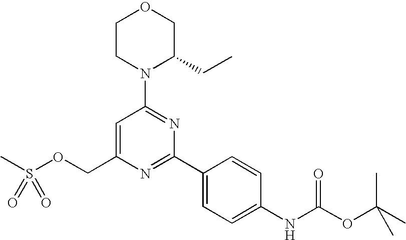

tert-Butyl N-[4-[4-[(3S)-3-ethylmorpholin-4-yl]-6-(hydroxymethyl)pyrimidin-2-yl]phenyl]carbamate (8.7 g, 20.99 mmol) and DIPEA (4.40 mL, 25.19 mmol) were added to DCM (80 mL), to this was slowly added methane sulphonyl chloride(1.636 mL, 20.99 mmol) and the reaction was stirred for 30 minutes. The reaction mixture was quenched with saturated ammonium chloride solution (100 mL), extracted with DCM (2×100 mL), the organic layer was dried over MgSO₄, filtered and evaporated to afford the desired material as a brown gum (10.2 g). This was used without any further purification.

LCMS Spectrum: m/z (ES+) (M+H)+=493; HPLC tR=2.90 min;

### tert-Butyl N-[4-[4-[(3S)-3-ethylmorpholin-4-yl]-6-(hydroxymethyl)pyrimidin-2-yl]phenylcarbamate

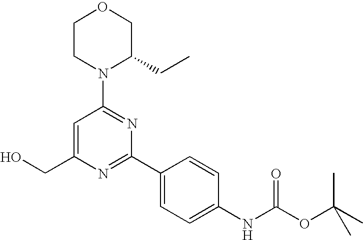

[2-Chloro-6-[(3S)-3-ethylmorpholin-4-yl]pyrimidin-4-yl]methanol (12 g, 46.56 mmol), tert-butyl 4-(4,4,5,5-tetramethyl-1,3,2-dioxaborolan-2-yl)phenylcarbamate (14.86 g, 46.56 mmol), sodium carbonate (24.68 g, 232.81 mmol) and 1,1′-bis(diphenyl phosphino)ferrocenedichloropalladium(II) (3.37 g, 4.66 mmol) were added to DME (150 mL) and water (37.5 mL) and heated to 90° C. overnight under nitrogen. The solvent was evaporated and the residue was quenched with water (100 mL), extracted with ethyl acetate (3×100 mL), the organic layer was dried over MgSO₄, filtered and evaporated to afford black gum. The residue was filtered through a plug of silica, eluting with ethyl acetate, to give a very dark gum. This was purified by flash silica chromatography, elution gradient 40 to 100% ethyl acetate in isohexane, to give an orange gum. The gum was dissolved in diethyl ether and isohexane carefully added until a cloudy solution was observed, further stirring gave the desired material as a white solid precipitate which was isolated by filtration (8.7 g).

NMR Spectrum: ¹H NMR (400.132 MHz, DMSO-d₆) δ 0.89 (3H, t), 1.49 (9H, s), 1.73-1.64 (1H, m), 1.84-1.77 (1H, m), 3.19 (1H, ddd), 3.47 (1H, ddd), 3.55 (1H, dd), 3.88 (1H, d), 3.94 (1H, dd), 4.33-4.23 (2H, m), 4.45 (2H, d), 5.38 (1H, t), 6.67 (1H, s), 7.53 (2H, d), 8.21 (2H, d), 9.50 (1H, s);

LCMS Spectrum: m/z (ES+) (M+H)+=415; HPLC tR=2.49 min;

### [2-Chloro-6-[(3S)-3-ethylmorpholin-4-yl]pyrimidin-4-yl]methanol

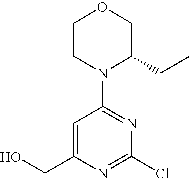

Lithium borohydride, 2M in THF (17.63 mL, 35.26 mmol) was added dropwise to methyl 2-chloro-6-[(3S)-3-ethylmorpholin-4-yl]pyrimidine-4-carboxylate (15.5 g, 54.25 mmol) in THF (100 mL) at 0° C. over a period of 30 minutes under nitrogen. The resulting solution was stirred at 0° C. for 30 minutes then allowed to warm to RT. Water (250 mL) was added and the THF evaporated. The aqueous residues were extracted with ethyl acetate (2×500 mL) and the combined organics were washed with water (2×300 mL). The organic layer was dried over MgSO₄ then evaporated to dryness to afford a viscous oil, this was triturated with hot diethyl ether to give the desired material as a white solid (13.4 g).

NMR Spectrum: ¹H NMR (400.132 MHz, DMSO-d₆) δ 0.84 (3H, t), 1.80-1.66 (2H, m), 3.19-3.14 (1H, m), 3.42 (1H, ddd), 3.51 (1H, dd), 3.82 (1H, d), 3.89 (1H, dd), 4.15-4.06 (2H, m), 4.34 (2H, d), 5.50 (1H, t), 6.74 (1H, s);

LCMS Spectrum: m/z (ES+) (M+H)+=258; HPLC tR=1.45 min;

### Methyl 2-chloro-6-[(3S)-3-ethylmorpholin-4-yl]pyrimidine-4-carboxylate

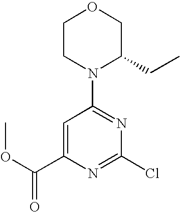

A solution of (S)-3-ethylmorpholine (10 g, 86.83 mmol) in DCM (75 mL) was added dropwise to a stirred solution of methyl 2,6-dichloropyrimidine-4-carboxylate (19.77 g, 95.51 mmol) and triethylamine (24.20 ml, 173.65 mmol) in DCM (200 mL) at RT, over a period of 2 hours under air. The resulting solution was stirred at RT overnight. The reaction mixture was quenched with water (250 mL), extracted with DCM (300 mL) and the solvent was removed to 30% the initial volume. The dark solution was passed through a 2 inch plug of silica, eluting with ethyl acetate, to give an orange gum which was dissolved in ethyl acetate (40 mL). To this was added diethyl ether (120 mL) and then isohexane until a cloudy solution was observed. The reaction was seeded with 15 mg of methyl 2-chloro-6-[(3S)-3-methylmorpholin-4-yl]pyrimidine-4-carboxylate and stirred for 15 minutes to afford the desired material as a white solid (15.8 g).

NMR Spectrum: ¹H NMR (400.132 MHz, DMSO-d₆) δ 0.83 (3H, t), 1.75 (2H, septet), 3.22 (1H, s), 3.44 (1H, ddd), 3.53 (1H, dd), 3.82 (1H, d), 3.91-3.87 (4H, m), 4.22 (2H, m), 7.32 (1H, s);

LCMS Spectrum: m/z (ES+) (M+H)+=286; HPLC tR=1.81 min;

### EXAMPLE 75

### 3-Cyclopropyl-1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]urea

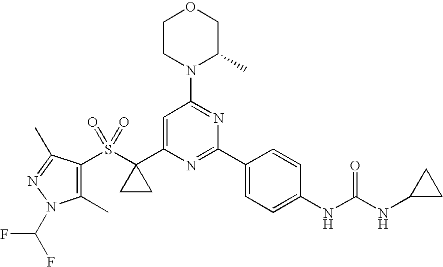

Cyclopropylamine (0.038 mL, 0.54 mmol) was added to a solution of phenyl N-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]carbamate (0.115g, 0.18 mmol) in NMP (2 mL) followed by triethylamine (0.076 mL, 0.54 mmol) and the reaction was heated at 50° C. overnight. The crude product was purified by preparative HPLC, eluting with decreasingly polar mixtures of water (containing 1% ammonia) and acetonitrile, to give the desired material as an off white solid (88 mg).

NMR Spectrum: ¹H NMR (399.9 MHz, DMSO-d₆) δ 0.40-0.44 (2H, m), 0.63-0.68 (2H, m), 1.19-1.21 (3H, d), 1.46-1.54 (2H, m), 1.75-1.78 (2H, m), 2.07 (3H, s), 2.34 (3H, s), 2.53-2.58 (1H, m), 3.12-3.20 (1H, m), 3.44-3.50 (1H, m), 3.60-3.64 (1H, m), 3.76 (1H, d), 3.95-3.99 (1H, m), 4.13 (1H, d), 4.51 (1H, s), 6.42 (1H, d), 6.69 (1H, s), 7.42-7.45 (2H, m), 7.64-7.93 (1H, t), 7.91-7.94 (2H, m), 8.53 (1H, s)

LCMS Spectrum: m/z (ESI+)(M+H)+=602; HPLC tR=2.20 min.

The compounds below were prepared in an analogous fashion from phenyl N-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 75a | 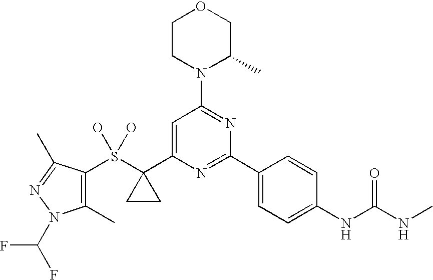 | 1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-methylurea | 576 | 2.05 |
| 75b | 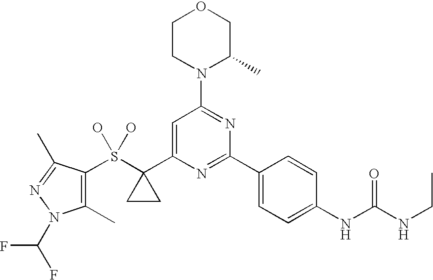 | 3-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-1-ethylurea | 590 | 2.19 |
| 75c | 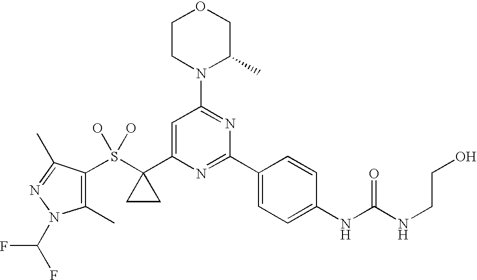 | 1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-(2-hydroxyethyl)urea | 606 | 1.89 |
| 75d | 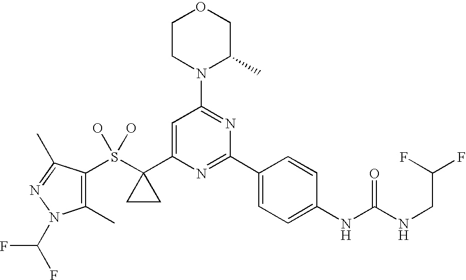 | 3-(2,2-difluoroethyl)-1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]urea | 626 | 2.31 |
| 75e | 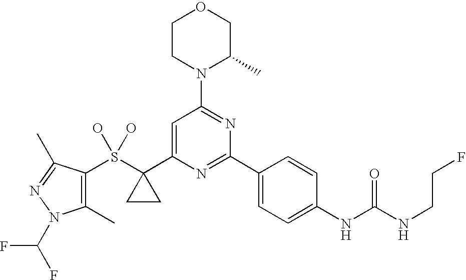 | 1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-(2-fluoroethyl)urea | 608 | 2.18 |
| 75f* | 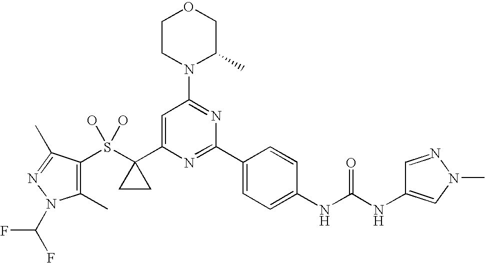 | 1-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]-3-(1-methylpyrazol-4-yl)urea | 642 | 2.08 |

*In addition to the reaction conditions described above this reaction was subsequently subjected to the addition of DIPEA (3 equivalents) and heating at 95° C. for 90 minutes.

### EXAMPLE 75a

¹H NMR (399.9 MHz, DMSO-d₆) δ 1.20 (3H, d), 1.46-1.54 (2H, m), 1.75-1.78 (2H, m), 2.07 (3H, s), 2.34 (3H, s), 2.68 (3H, d), 3.12-3.17 (1H, m), 3.44-3.50 (1H, m), 3.60-3.64 (1H, m), 3.76 (1H, d), 3.95-3.99 (1H, m), 4.13 (1H, d), 4.51 (1H, s), 6.05 (1H, q), 6.69 (1H, s), 7.42-7.44 (2H, m), 7.64-7.92 (1H, t), 7.90-7.93 (2H, m), 8.73 (1 H, s).

### EXAMPLE 75b

¹H NMR (399.9 MHz, DMSO-d₆) δ 1.07 (3H, t), 1.20 (3H, d), 1.49-1.51 (2H, m), 1.75-1.78 (2H, m), 2.07 (3H, s), 2.34 (3H, s), 3.10-3.20 (3H, m), 3.44-3.50 (1H, m), 3.60-3.64 (1H, m), 3.76 (1H, d), 3.95-3.99 (1H, m), 4.11-4.15 (1H, m), 4.51 (1H, s), 6.14 (1H, t), 6.69 (1H, s), 7.41-7.44 (2H, m), 7.64-7.92 (1H, t), 7.91-7.93 (2H, d), 8.65 (1H, s).

### EXAMPLE 75c

¹H NMR (399.9 MHz, DMSO-d₆) δ 1.20 (3H, s), 1.46-1.54 (2H, m), 1.75-1.78 (2H, m), 2.07 (3H, s), 2.34 (3H, s), 3.12-3.20 (3H, m), 3.45-3.50 (3H, m), 3.60-3.64 (1H, m), 3.76 (1H, d), 3.95-3.99 (1H, m), 4.13 (1H, d), 4.51 (1H, s), 4.74 (1H, t), 6.24 (1H, t), 6.69 (1H, s), 7.42 (2H, d), 7.64-7.92 (1H, t), 7.92 (2H, d), 8.79 (1H, s).

### EXAMPLE 75d

¹H NMR (399.9 MHz, DMSO-d₆) δ 1.20 (3H, d), 1.46-1.54 (2H, m), 1.75-1.78 (2H, m), 2.07 (3H, s), 2.34 (3H, s), 3.16-3.19 (1H, m), 3.44-3.64 (4H, m), 3.76 (1H, d), 3.95-3.99 (1H, m), 4.12-4.15 (1H, m), 4.51 (1H, s), 5.93-6.23 (1H, m), 6.52 (1H, t), 6.70 (1H, s), 7.44 (2H, d), 7.64-7.92 (1H, t), 7.95 (2H, d), 8.92 (1H, s).

### EXAMPLE 75e

¹H NMR (399.9 MHz, DMSO-d₆) δ 1.20 (3H, d), 1.46-1.54 (2H, m), 1.73-1.78 (2H, m), 2.07 (3H, s), 2.34 (3H, s), 3.14-3.20 (1H, m), 3.36-3.50 (3H, m), 3.60-3.64 (1H, m), 3.76 (1H, d), 3.95-3.99 (1H, m), 4.13 (1H, d), 4.42 (1H, t), 4.54 (2H, t), 6.42 (1H, t), 6.70 (1H, s), 7.43 (2H, d), 7.64-7.93 (1H, t), 7.93 (2H, d), 8.80 (1H, s).

### EXAMPLE 75f

¹H NMR (399.9 MHz, DMSO-d₆) δ 1.20-1.21 (3H, m), 1.47-1.54 (2H, m), 1.74-1.79 (2H, m), 2.07 (3H, s), 2.35 (3H, s), 3.13-3.21 (1H, m), 3.44-3.51 (1H, m), 3.61-3.64 (1H, m), 3.75-3.78 (1H, m), 3.80 (3H, s), 3.95-3.99 (1H, m), 4.14 (1H, d), 4.52 (1H, s), 6.70 (1H, s), 7.39-7.39 (1H, m), 7.46-7.49 (2H, m), 7.78 (1H, s), 7.65-7.94 (1H, t), 7.96 (2H, d), 8.37 (1H, s), 8.84 (1H, s)

The preparation of phenyl N-[4-[4-[1-[1-(difluoromethyl)-3,5-dimethylpyrazol-4-yl]sulfonylcyclopropyl]-6-[(3S)-3-methylmorpholin-4-yl]pyrimidin-2-yl]phenyl]carbamate is described below.

### Phenyl N-[4-[4-[1-[1-(difluoromethyl)-3 5-dimethylpyrazol-4-yl]sulfonylcyclopropyl]-6-[(3S)-3-methylmorpholin-4-yl]pyrimidin-2-yl]phenyl]carbamate

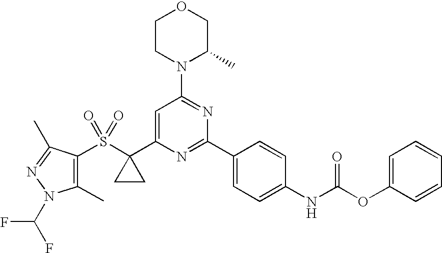

Phenyl chloroformate (0.176 mL, 1.40 mmol) was added dropwise to a mixture of 4-[4-[1-[1-(difluoromethyl)-3,5-dimethylpyrazol-4-yl]sulfonylcyclopropyl]-6-[(3S)-3-methylmorpholin-4-yl]pyrimidin-2-yl]aniline (0.726 g, 1.4 mmol) and sodium hydrogen carbonate (0.176 g, 2.10 mmol) in dioxane (20 mL) under nitrogen. The resulting suspension was stirred at RT for 2 hours. The precipitate was collected by filtration, washed with a mixture of isohexane (4 mL) and diethyl ether (2 mL), then suspended in water (20 mL) and stirred for 20 minutes. The precipitate was collected by filtration, washed with water (5 mL) then with a mixture of isohexane (10 mL ) and diethyl ether (5 mL) and dried under vacuum to afford the desired material as a white solid (0.826 g).

NMR Spectrum: ¹H NMR (399.9 MHz, DMSO-d₆) δ 1.21 (3H, d), 1.48-1.55 (2H, m), 1.74-1.79 (2H, m), 2.07 (3H, s), 2.35 (3H, s), 3.14-3.17 (1H, m), 3.39-3.50 (1H, m), 3.61-3.64 (1H, m), 3.77 (1H, m), 3.95-3.99 (1H, m), 4.16 (1H, d), 4.53 (1H, s), 6.75 (1H, s), 7.24-7.31 (3H, m), 7.44-7.48 (2H, m), 7.57 (2H, d), 7.64-7.93 (1H, t), 8.00-8.03 (2H, m), 10.42 (1H, s).

LCMS Spectrum: m/z (ESI+)(M+H)+=639; HPLC tR=2.92 min.

### 4-[4-[1-[1-(Difluoromethyl)-3,5-dimethylpyrazol-4-yl]sulfonylcyclopropyl]-6-[(3S)-3-methylmorpholin-4-yl]pyrimidin-2-yl]aniline

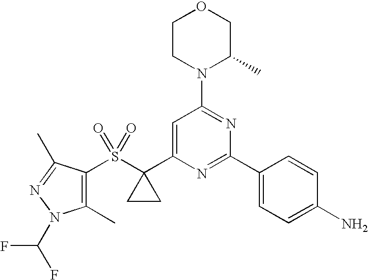

Dichlorobis(triphenylphosphine)palladium(II) (0.412 g, 0.59 mmol) was added in one portion to a mixture of sodium carbonate (1.867 g, 17.61 mmol), 2-chloro-4-[1-[1-(difluoromethyl)-3,5-dimethylpyrazol-4-yl]sulfonylcyclopropyl]-6-[(3S)-3-methylmorpholin-4-yl]pyrimidine (2.712 g, 5.87 mmol) and (4-aminophenyl)boronic acid pinacol ester (1.351 g, 6.16 mmol) in DME (50 mL)and water (12.5 mL) under nitrogen. The mixture was stirred at 80° C. for 2 hours, cooled to RT then diluted with DCM (100 mL) and water (50 mL). The organic layer was washed with saturated brine (50 mL) then dried over MgSO₄, filtered and evaporated to afford crude product. The crude product was purified by flash silica chromatography, elution gradient 30 to 60% ethyl acetate in isohexane, to give a residue which was further purified by trituration with isohexane (25 mL) and diethyl ether (25 mL), to give the desired material as a yellow solid (2.84 g).

NMR Spectrum: ¹H NMR (399.9 MHz, DMSO-d₆) δ 1.18 (3H, m), 1.43-1.51 (2H, m), 1.73-1.76 (2H, m), 2.07 (3H, s), 2.34 (3H, s), 3.09-3.17 (1H, m), 3.42-3.49 (1H, m), 3.59-3.63 (1H, m), 3.75 (1H, d), 3.94-3.97 (1H, m), 4.48 (1H, s), 5.52 (2H, m), 6.52-6.55 (2H, m), 6.58 (1H, s), 7.75-7.78 (2H, m), 7.64-7.93 (1H, t).

LCMS Spectrum: m/z (ESI+)(M+H)+=519; HPLC tR=1.91 min.

### 2-Chloro-4-[1-[1-(difluoromethyl)-3,5-dimethylpyrazol-4-yl]sulfonylcyclopropyl]-6-[(3S)-3-methylmorpholin-4-yl]pyrimidine

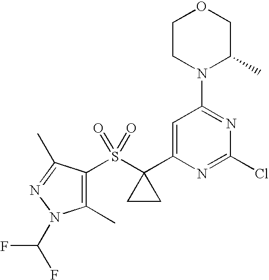

Sodium hydroxide (33.0 mL, 412.50 mmol) was added to 2-chloro-4-[[1-(difluoromethyl)-3,5-dimethylpyrazol-4-yl]sulfonylmethyl]-6-[(3S)-3-methylmorpholin-4-yl]pyrimidine (3.27 g, 7.5 mmol), 1,2-dibromoethane (1.939 mL, 22.50 mmol) and tetrabutylammonium bromide (0.242 g, 0.75 mmol) in toluene (132 mL). The resulting solution was stirred at 60° C. for 1 hour. The reaction mixture was diluted with DCM (200 mL), and washed twice with water (200 mL). The organic layer was dried over MgSO₄, filtered and evaporated to afford crude product. The crude product was purified by flash silica chromatography, elution gradient 0 to 20% ethyl acetate in DCM, to give the desired material as a white solid (2.77 g).

NMR Spectrum: ¹H NMR (399.9 MHz, DMSO-d₆) δ 1.18 (3H, d), 1.45-1.52 (2H, m), 1.69-1.76 (2H, m), 2.13 (3H, s), 2.37 (3H, s), 3.12-3.19 (1H, m), 3.37-3.44 (1H, m), 3.53-3.57 (1H, m), 3.71 (1H, d), 3.90-3.94 (2H, m), 4.35 (1H, s), 6.80 (1H, s), 7.71-8.00 (1H, t).

LCMS Spectrum: m/z (ESI+)(M+H)+=462; HPLC tR=2.26 min.

### 2-Chloro-4-[[1-(difluoromethyl)-3,5-dimethylpyrazol-4-yl]sulfonylmethyl]-6-[(3S)-3-methylmorpholin-4-yl]pyrimidine

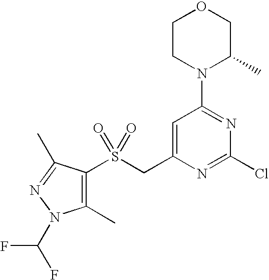

Sodium 1-(difluoromethyl)-3,5-dimethylpyrazole-4-sulfinate (3.44 g, 14.83 mmol) was added to a solution of 2-chloro-4-(iodomethyl)-6-[(3S)-3-methylmorpholin-4-yl]pyrimidine (4.37 g, 12.36 mmol) in DMF (20 mL). The resulting mixture was stirred at RT for 1 hour. The reaction mixture was evaporated to dryness, redissolved in DCM (150 mL), and washed sequentially with water (100 mL) and saturated brine (50 mL). The organic layer was dried over MgSO₄, filtered and evaporated to afford crude product. The crude product was purified by flash silica chromatography, elution gradient 20 to 60% ethyl acetate in isohexane, to give the desired material as a white solid (5.22 g).

NMR Spectrum: ¹H NMR (399.9 MHz, DMSO-d₆) δ 1.19 (3H, d), 2.21 (3H, s), 2.42 (3H, s), 3.15-3.22 (1H, m), 3.40-3.47 (1H, m), 3.57-3.60 (1H, m), 3.73 (1H, d), 3.92-3.96 (2H, m), 4.25 (1H, s), 4.57 (2H, s), 6.82 (1H, s), 7.73-8.02 (1H, t).

LCMS Spectrum: m/z (ESI+)(M+H)+=436; HPLC tR=2.13 min.

### Sodium 1-(difluoromethyl)-3 5-dimethylpyrazole-4-sulfinate

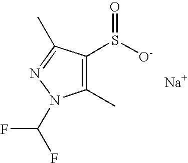

Sodium hydrogen carbonate (3.43 g, 40.88 mmol) was added to a solution of sodium sulfite (2.58 g, 20.44 mmol) in water (25 mL) and the resulting solution was stirred at 50° C. for 1 hour. 1-(Difluoromethyl)-3,5-dimethyl-1H-pyrazole-4-sulfonyl chloride (5 g, 20.44 mmol) was added portionwise and the solution was stirred at 50° C. for 18 hours. The reaction mixture was evaporated to dryness and methanol (75 mL) was added. The suspension was allowed to stir at RT for 20 minutes, filtered and the filtrate was evaporated. The residue was dissolved in ethanol (50 mL) at 50° C., filtered and evaporated to afford the desired material as a white solid (4.59 g).

NMR Spectrum: ¹H NMR (399.9 MHz, DMSO-d₆) δ 2.24 (3H, s), 2.46 (3H, s), 7.44-7.74-7.74 (1H, t).

The preparation of 2-chloro-4-(iodomethyl)-6-[(3S)-3-methylmorpholin-4-yl]pyrimidine was described earlier.

### EXAMPLE 76

### 1-[4-[4-[1-(2-Chlorophenyl)sulfonylcyclopropyl]-6-[(3S)-3-methylmorpholin-4-yl]pyrimidin-2-yl]phenyl]-3-cyclopropylurea

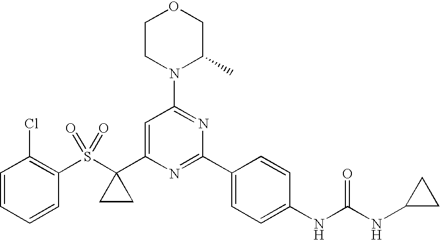

Cyclopropylamine (0.037 mL, 0.51 mmol) was added to a solution of phenyl N-[4-[4-[1-(2-chlorophenyl)sulfonylcyclopropyl]-6-[(3S)-3-methylmorpholin-4-yl]pyrimidin-2-yl]phenyl]carbamate (0.103 g, 0.17 mmol) in NMP (2 mL) followed by triethylamine (0.072 mL, 0.51 mmol) and the reaction was heated at 50° C. overnight. The crude product was purified by preparative HPLC using decreasingly polar mixtures of water (containing 1% NH3) and MeCN as eluents, to give the desired material as an off-white solid (82 mg, 80%).

NMR Spectrum: ¹H NMR (399.9 MHz, DMSO-d₆) δ 0.40-0.44 (2H, m), 0.63-0.68 (2H, m), 1.16 (3H, d), 1.71-1.75 (2H, m), 1.97-2.01 (2H, m), 2.53-2.57 (1H, m), 3.12-3.16 (1H, m), 3.41-3.48 (1H, m), 3.58-3.61 (1H, m), 3.74 (1H, d), 3.93-3.97 (1H, m), 4.10 (1H, d), 4.41 (1H, s), 6.42 (1H, d), 6.63 (1H, s), 7.37-7.40 (2H, m), 7.43-7.46 (1H, m), 7.60-7.64 (1H, m), 7.67-7.69 (1H, m), 7.78-7.82 (2H, m), 7.90-7.92 (1H, m), 8.50 (1H, s).

LCMS Spectrum: m/z (ESI+)(M+H)+=568; HPLC tR=2.30 min.

The compounds below were prepared in an analogous fashion from phenyl N-[4-[4-[1-(2-chlorophenyl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 76a | 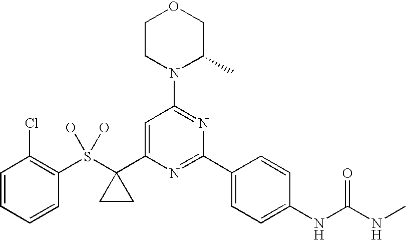 | 1-[4-[4-[1-(2-chlorophenyl)sulfonylcyclopropyl]-6-[(3S)-3-methylmorpholin-4-yl]pyrimidin-2-yl]phenyl]-3-methylurea | 542 | 2.16 |
| 76b | 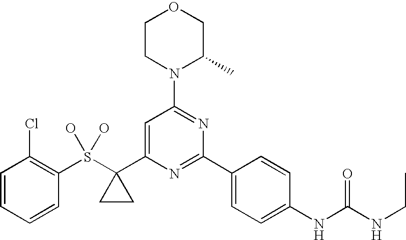 | 3-[4-[4-[1-(2-chlorophenyl)sulfonylcyclopropyl]-6-[(3S)-3-methylmorpholin-4-yl]pyrimidin-2-yl]phenyl]-1-ethylurea | 556 | 2.30 |
| 76c | 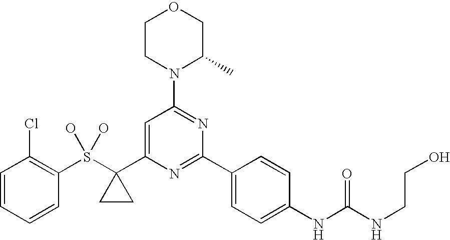 | 1-[4-[4-[1-(2-chlorophenyl)sulfonylcyclopropyl]-6-[(3S)-3-methylmorpholin-4-yl]pyrimidin-2-yl]phenyl]-3-(2-hydroxyethyl)urea | 572 | 1.99 |
| 76d | 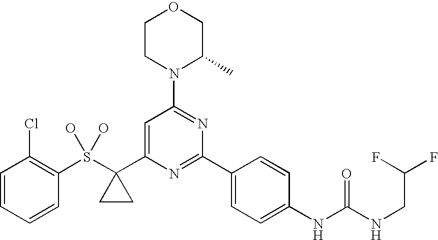 | 1-[4-[4-[1-(2-chlorophenyl)sulfonylcyclopropyl]-6-[(3S)-3-methylmorpholin-4-yl]pyrimidin-2-yl]phenyl]-3-(2,2-difluoroethyl)urea | 592 | 2.40 |
| 76e | 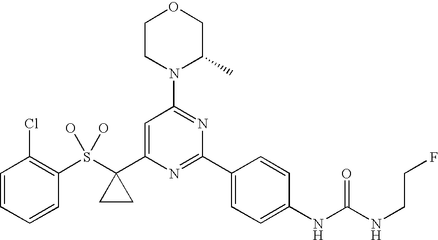 | 1-[4-[4-[1-(2-chlorophenyl)sulfonylcyclopropyl]-6-[(3S)-3-methylmorpholin-4-yl]pyrimidin-2-yl]phenyl]-3-(2-fluoroethyl)urea | 574 | 2.27 |
| 76f* | 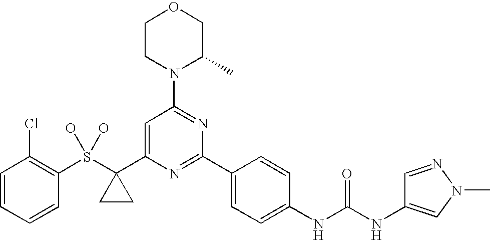 | 1-[4-[4-[1-(2-chlorophenyl)sulfonylcyclopropyl]-6-[(3S)-3-methylmorpholin-4-yl]pyrimidin-2-yl]phenyl]-3-(1-methylpyrazol-4-yl)urea | 608 | 2.12 |

*In addition to the reaction conditions described above this reaction was subsequently subjected to the addition of DIPEA (3 equivalents) and heating at 95° C. for 90 minutes.

### EXAMPLE 76a

¹H NMR (399.9 MHz, DMSO-d₆) δ 1.15-1.16 (3H, m), 1.73 2H, m), 1.97-2.01 (2H, m), 2.66 (3H, d), 3.12 (1H, d), 3.40-3.48 (1H, m), 3.58-3.61 (1H, m), 3.74 (1H, d), 3.93-3.97 (1H, m), 4.08 (1H, m), 4.41 (1H, s), 6.06 (1H, d), 6.62 (1H, s), 7.38 (2H, d), 7.46 (1H, m), 7.60-7.62 (1H, m), 7.67-7.69 (1H, m), 7.79 (2H, d), 7.90-7.92 (1H, d), 8.70 (1H, s).

### EXAMPLE 76b

¹H NMR (399.9 MHz, DMSO-d₆) δ 1.07 (3H, t), 1.16 (3H, d), 1.72-1.75 (2H, m), 1.97-2.01 (2H, m), 3.10-3.16 (3H, m), 3.42-3.45 (1H, m), 3.58-3.61 (1H, m), 3.73-3.75 (1H, m), 3.93-3.98 (1H, m), 4.10 (1H, d), 4.41 (1H, s), 6.15 (1H, t), 6.63 (1H, s), 7.35-7.39 (2H, m), 7.43-7.47 (1H, m), 7.60-7.64 (1H, m), 7.67-7.70 (1H, m), 7.78-7.81 (2H, d), 7.90-7.92 (1H, m), 8.62 (1H, s).

### EXAMPLE 76c

¹H NMR (399.9 MHz, DMSO-d₆) δ 1.15-1.16 (3H, m), 1.73 (2H, m), 1.97-2.01 (2H, m), 3.16 (1H, m), 3.17-3.19 (2H, m), 3.46 (3H, m), 3.58-3.61 (1H, m), 3.74 (1H, d), 3.93-3.97 (1H, m), 4.10 (1H, m), 4.42 (1H, s), 4.74 (1H, t), 6.24 (1H, t), 6.63 (1H, s), 7.36 (2H, d), 7.42-7.47 (1H, m), 7.60-7.62 (1H, m), 7.67-7.70 (1H, m), 7.80 (2H, d), 7.90-7.92 (1H, m), 8.76 (1H, s).

### EXAMPLE 76d

¹H NMR (399.9 MHz, DMSO-d₆) δ 1.16 (3H, d), 1.70-1.77 (2H, m), 1.96-2.03 (2H, m), 3.09-3.16 (1H, m), 3.41-3.48 (1H, m), 3.49-3.62 (3H, m), 3.74 (1H, d), 3.93-3.97 (1H, m), 4.08-4.12 (1H, d), 4.41 (1H, s), 5.92-6.22 (1H, m), 6.52 (1H, t), 6.64 (1H, s), 7.37-7.41 (2H, d), 7.44-7.47 (1H, m), 7.60-7.62 (1H, m), 7.67-7.70 (1H, m), 7.82 (2H, d), 7.90-7.92 (1H, m), 8.88 (1H, s).

### EXAMPLE 76e

¹H NMR (399.9 MHz, DMSO-d₆) δ 1.16 (3H, d), 1.69-1.77 (2H, m), 1.95-2.03 (2H, m), 3.09-3.16 (1H, m), 3.36-3.48 (3H, m), 3.58-3.62 (1H, m), 3.74 (1H, d), 3.93-3.97 (1H, m), 4.10 (1H, d), 4.42 (2H, m), 4.54 (1H, t), 6.42 (1H, t), 6.63 (1H, s), 7.36-7.40 (2H, m), 7.43-7.47 (1H, m), 7.60-7.66 (1H, m), 7.67-7.70 (1H, m), 7.80-7.82 (2H, m), 7.90-7.92 (1H, m), 8.77 (1H, s).

### EXAMPLE 76f

¹H NMR (399.9 MHz, DMSO-d₆) δ 1.10-1.17 (3H, d), 1.72-1.75 (2H, m), 1.98-2.01 (2H, m), 3.13-3.16 (1H, m), 3.42-3.48 (1H, m), 3.58-3.62 (1H, m), 3.75 (1H, d), 3.80 (3H, s), 3.94-3.97 (1H, m), 4.12 (1H, m), 4.42 (1H, s), 6.64 (1H, s), 7.40-7.47 (4H, m), 7.61-7.63 (1H, m), 7.68-7.71 (1H, m), 7.77 (1H, s), 7.83 (2H, d), 7.90-7.93 (1H, m), 8.38 (1H, s), 8.80 (1H, s).

The preparation of phenyl N-[4-[4-[1-(2-chlorophenyl)sulfonylcyclopropyl]-6-[(3S)-3-methylmorpholin-4-yl]pyrimidin-2-yl]phenyl]carbamate is described below. Phenyl N-[4-[4-[1-(2-chlorophenyl)sulfonylcyclopropyl]-6-[(3S)-3-methylmorpholin-4-yl]pyrimidin-2-yl]phenyl]carbamate

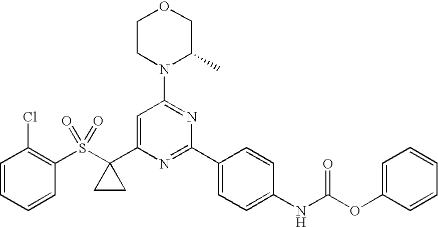

Phenyl chloroformate (0.189 mL, 1.50 mmol) was added dropwise to a mixture of 4-[4-[1-(2-chlorophenyl)sulfonylcyclopropyl]-6-[(3S)-3-methylmorpholin-4-yl]pyrimidin-2-yl]aniline (0.727 g, 1.5 mmol) and sodium hydrogen carbonate (0.189 g, 2.25 mmol) in dioxane (20 mL) under nitrogen. The resulting suspension was stirred at RT for 2 hours. The precipitate was collected by filtration, washed with diethyl ether (10 mL), suspended in water (20 mL), stirred for 20 minutes, collected by filtration, washed with water (10 mL) then ether (2 mL) and dried under vacuum to afford the desired material as a white solid (0.735 g).

NMR Spectrum: ¹H NMR (399.9 MHz, DMSO-d₆) δ 1.17 (3H, d), 1.72-1.77 (2H, m), 1.98-2.01 (2H, m), 3.10-3.18 (1H, m), 3.44-3.62 (2H, m), 3.74 (1H, d), 3.93-3.97 (1H, m), 4.12 (1H, d), 4.43 (1H, s), 6.67 (1H, s), 7.24-7.31 (3H, m), 7.43-7.48 (3H, m), 7.51-7.53 (2H, m), 7.60-7.63 (1H, m), 7.67-7.70 (1H, m), 7.88-7.92 (3H, m), 10.40 (1H, s).

LCMS Spectrum: m/z (ESI+)(M+H)+=605; HPLC tR=3.02 min.

### 4-[4-[1-(2-Chlorophenyl)sulfonylcyclopropyl]-6-[(3S)-3-methylmorpholin-4-yl]pyrimidin-2-yl]aniline

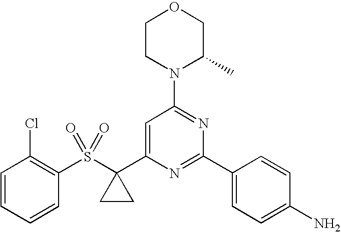

Dichlorobis(triphenylphosphine)palladium(II) (0.519 g, 0.74 mmol) was added in one portion to a mixture of sodium carbonate (2.352 g, 22.20 mmol), 2-chloro-4-[1-(2-chlorophenyl)sulfonylcyclopropyl]-6-[(3S)-3-methylmorpholin-4-yl]pyrimidine (3.169 g, 7.40 mmol) and (4-aminophenyl)boronic acid pinacol ester (1.702 g, 7.77 mmol) in DME (56 mL)and water (14 mL) under nitrogen. The mixture was stirred at 80° C. for 2 hours, cooled to RT then diluted with DCM (100 mL) and water (50 mL). The organic layer was washed with saturated brine (50 mL) then dried over MgSO₄, filtered and evaporated to afford crude product. The crude product was purified by flash silica chromatography, elution gradient 30 to 60% ethyl acetate in isohexane, to give a residue which was further purified by trituration with isohexane (25 mL) and diethyl ether (25 mL), to give the desired material as a yellow solid (3.21 g).

NMR Spectrum: ¹H NMR (399.9 MHz, DMSO-d₆) δ 1.08-1.19 (3H, m), 1.67-1.75 (2H, m), 1.97-2.00 (2H, m), 3.05-3.13 (1H, m), 3.39-3.43 (1H, m), 3.56-3.60 (1H, m), 3.73 (1H, d), 3.91-3.96 (1H, m), 4.04 (1H, q), 4.37 (1H, s), 5.49 (2H, m), 6.47-6.49 (2H, m), 6.52 (1H, s), 7.43-7.47 (1H, m), 7.59-7.68 (4H, m), 7.90-7.93 (1H, m).

LCMS Spectrum: m/z (ESI+)(M+H)+=485; HPLC tR=1.94 min.

### 2-Chloro-4-[1-(2-chlorophenyl)sulfonylcyclopropyl]-6-[(3S)-3-methylmorpholin-4-yl]pyrimidine

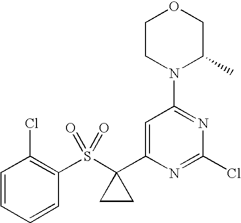

Sodium hydroxide (50% w/w solution) (35.2 mL, 440.00 mmol) was added to 2-chloro-4-[(2-chlorophenyl)sulfonylmethyl]-6-[(3S)-3-methylmorpholin-4-yl]pyrimidine (3.22 g, 8 mmol), 1,2-dibromoethane (2.068 mL, 24.00 mmol) and tetrabutylammonium bromide (0.258 g, 0.80 mmol) in toluene (141 mL). The resulting solution was stirred at 60° C. for 18 hours. The reaction mixture was diluted with DCM (150 mL), and washed twice with water (150 mL). The organic layer was dried over MgSO₄, filtered and evaporated to afford crude product. The crude product was purified by flash silica chromatography, elution gradient 0 to 10% ethyl acetate in DCM, to give the desired material as a colourless dry film (3.23 g).

NMR Spectrum: ¹H NMR (399.9 MHz, DMSO-d₆) δ 1.13 (3H, d), 1.64-1.67 (2H, m), 1.92-1.95 (2H, m), 3.07-3.15 (1H, m), 3.35-3.41 (1H, m), 3.51-3.55 (1H, m), 3.69 (1H, d), 3.88-3.92 (2H, m), 4.24 (1H, s), 6.72 (1H, s), 7.52-7.56 (1H, m), 7.66-7.73 (2H, m), 7.88-7.91 (1H, m).

LCMS Spectrum: m/z (ESI+)(M+H)+=428; HPLC tR=2.42 min.

### 2-Chloro-4-[(2-chlorophenyl)sulfonylmethyl]-6-[(3S)-3-methylmorpholin-4-yl]pyrimidine

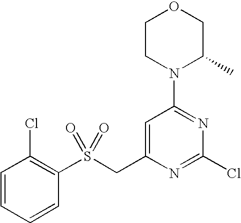

Sodium 2-chlorobenzenesulfinate (3.19 g, 16.07 mmol) was added to a solution of 2-chloro-4-(iodomethyl)-6-[(3S)-3-methylmorpholin-4-yl]pyrimidine (4.37 g, 12.36 mmol) in DMF (20 mL). The resulting mixture was stirred at RT for 1 hour. The reaction mixture was evaporated to dryness and redissolved in DCM (150 mL) and washed sequentially with water (100 mL) and saturated brine (50 mL). The organic layer was dried over MgSO₄, filtered and evaporated to afford crude product. The crude product was purified by flash silica chromatography, elution gradient 20 to 60% ethyl acetate in isohexane, to give the desired material as a white solid (4.34 g).

NMR Spectrum: ¹H NMR (399.9 MHz, DMSO-d₆) δ 1.16-1.18 (3H, d), 3.13-3.20 (1H, m), 3.39-3.46 (1H, m), 3.56-3.59 (1H, m), 3.72 (1H, d), 3.91-3.95 (2H, m), 4.22 (1H, s), 4.78 (2H, s), 6.79 (1H, s), 7.56-7.59 (1H, m), 7.74-7.79 (2H, m), 7.82-7.84 (1H, m).

LCMS Spectrum: m/z (ESI+)(M+H)+=402; HPLC tR=2.26 min.

The preparation of 2-chloro-4-(iodomethyl)-6-[(3S)-3-methylmorpholin-4-yl]pyrimidine was described earlier.

### EXAMPLE 77

### 1-[4-[4-[1-(4-Cyanophenyl)sulfonylcyclopropyl]-6-[(3S)-3-methylmorpholin-4-yl]pyrimidin-2-yl]phenyl]-3-cyclopropylurea

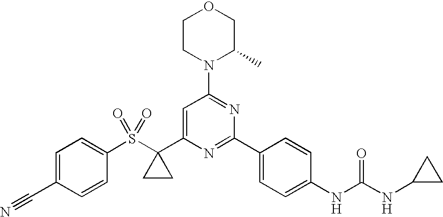

Cyclopropylamine (32 mg, 0.564 mmol) was added to phenyl N-[4-[4-[1-(4-cyanophenyl)sulfonylcyclopropyl]-6-[(3S)-3-methylmorpholin-4-yl]pyrimidin-2-yl]phenyl]carbamate (112 mg, 0.188 mmol) and triethylamine (0.080 mL, 0.564 mmol) in NMP (2 mL). The resulting solution was stirred at 50° C. for 16 hours then cooled to RT and purified by preparative HPLC, eluting with decreasingly polar mixtures of water (containing 1% ammonia) and acetonitrile, to give the desired material as a solid (81 mg).

NMR Spectrum: ¹H NMR (399.902 MHz, DMSO-d₆) δ 0.42 (2H, m), 0.65 (2H, m), 1.19 (3H, d), 1.65 (2H, m), 1.96 (2H, m), 2.56 (1H, m), 3.17 (1H, m), 3.47 (1H, m), 3.61 (1H, m), 3.75 (1H, m), 3.96 (1H, m), 4.16 (1H, m), 4.46 (1H, m), 6.41 (1H, d), 6.67 (1H, s), 7.39 (2H, d), 7.68 (2H, d), 7.98 (2H, d), 8.08 (2H, d), 8.53 (1H, s).

LCMS Spectrum: m/z (ESI+)(M+H)+=559; HPLC tR=2.36 min.

The compounds below were prepared in an analogous fashion from phenyl N-[4-[4-[1-(4-cyanophenyl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 77a | 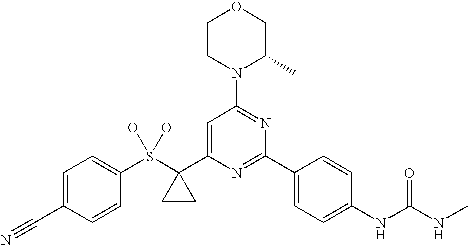 | 1-[4-[4-[1-(4-cyanophenyl)sulfonylcyclopropyl]-6-[(3S)-3-methylmorpholin-4-yl]pyrimidin-2-yl]phenyl]-3-methylurea | 533 | 2.20 |
| 77b | 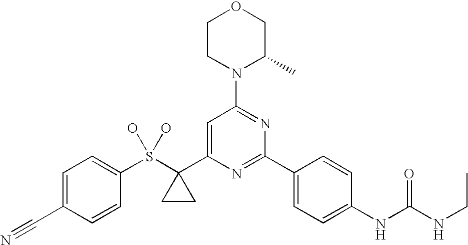 | 3-[4-[4-[1-(4-cyanophenyl)sulfonylcyclopropyl]-6-[(3S)-3-methylmorpholin-4-yl]pyrimidin-2-yl]phenyl]-1-ethylurea | 547 | 2.34 |
| 77c | 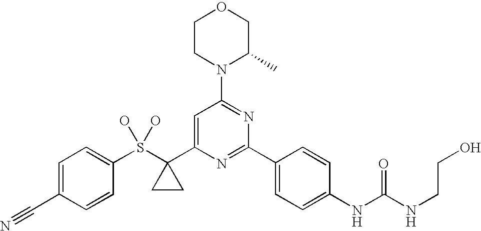 | 1-[4-[4-[1-(4-cyanophenyl)sulfonylcyclopropyl]-6-[(3S)-3-methylmorpholin-4-yl]pyrimidin-2-yl]phenyl]-3-(2-hydroxyethyl)urea | 563 | 2.00 |
| 77d | 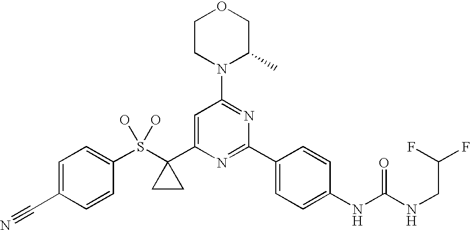 | 1-[4-[4-[1-(4-cyanophenyl)sulfonylcyclopropyl]-6-[(3S)-3-methylmorpholin-4-yl]pyrimidin-2-yl]phenyl]-3-(2,2-difluoroethyl)urea | 583 | 2.46 |
| 77e | 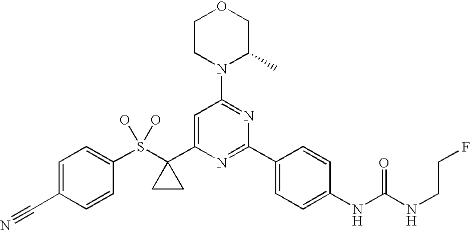 | 1-[4-[4-[1-(4-cyanophenyl)sulfonylcyclopropyl]-6-[(3S)-3-methylmorpholin-4-yl]pyrimidin-2-yl]phenyl]-3-(2-fluoroethyl)urea | 565 | 2.34 |
| 77f | 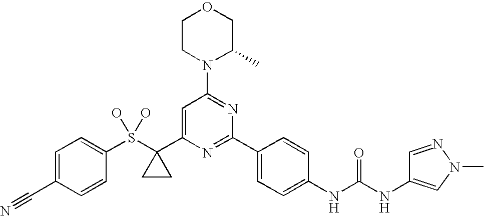 | 1-[4-[4-[1-(4-cyanophenyl)sulfonylcyclopropyl]-6-[(3S)-3-methylmorpholin-4-yl]pyrimidin-2-yl]phenyl]-3-(1-methylpyrazol-4-yl)urea | 599 | 2.22 |

*In addition to the reaction conditions described above this sample was further subjected to the addition of more triethylamine (3 equivalents) and stirred for an additional 7 hours at 80° C.

### EXAMPLE 77a

¹H NMR (399.902 MHz, DMSO-d₆) δ 1.19 (3H, d), 1.65 (2H, m), 1.95 (2H, m), 2.66 (3H, d), 3.16 (1H, m), 3.47 (1H, m), 3.61 (1H, m), 3.75 (1H, m), 3.96 (1H, m), 4.16 (1H, m), 4.46 (1H, m), 6.06 (1H, q), 6.66 (1H, s), 7.39 (2H, d), 7.68 (2H, d), 7.98 (2H, d), 8.08 (2H, d), 8.73 (1H, s).

### EXAMPLE 77b

¹H NMR (399.902 MHz, DMSO-d₆) δ 1.07 (3H, t), 1.19 (3H, d), 1.65 (2H, m), 1.95 (2H, m), 3.09-3.20 (3H, m), 3.47 (1H, m), 3.62 (1H, m), 3.75 (1H, m), 3.96 (1H, m), 4.16 (1H, m), 4.46 (1H, m), 6.15 (1H, t), 6.66 (1H, s), 7.38 (2H, d), 7.67 (2H, d), 7.98 (2H, d), 8.08 (2H, d), 8.65 (1H, s).

### EXAMPLE 77c

¹H NMR (399.902 MHz, DMSO-d₆) δ 1.20 (3H, d), 1.65 (2H, m), 1.96 (2H, m), 3.18 (3H, m), 3.47 (3H, m), 3.61 (1H, m), 3.75 (1H, m), 3.96 (1H, m), 4.16 (1H, m), 4.46 (1H, m), 4.74 (1H, t), 6.25 (1H, t), 6.66 (1H, s), 7.37 (2H, d), 7.67 (2H, d), 7.98 (2H, d), 8.09 (2H, d), 8.79 (1H, s).

### EXAMPLE 77d

¹H NMR (399.902 MHz, DMSO-d₆) δ 1.20 (3H, d), 1.65 (2H, m), 1.96 (2H, m), 3.17 (1H, m), 3.43-3.63 (4H, m), 3.75 (1H, m), 3.96 (1H, m), 4.16 (1H, m), 4.46 (1H, m), 5.92-6.22 (1H, m), 6.52 (1H, t), 6.67 (1H, s), 7.39 (2H, d), 7.70 (2H, d), 8.08 (2H, d), 8.91 (1H, s).

### EXAMPLE 77e

¹H NMR (399.902 MHz, DMSO-d₆) δ 1.20 (3H, d), 1.65 (2H, m), 1.96 (2H, m), 3.16 (1H, m), 3.39 (1H, m), 3.47 (2H, m), 3.61 (1H, m), 3.75 (1H, m), 3.96 (1H, m), 4.16 (1H, m), 4.41-4.55 (3H, m), 6.43 (1H, t), 6.67 (1H, s), 7.39 (2H, d), 7.69 (2H,d), 7.98 (2H, d), 8.09 (2H, d), 8.80 (1H, s).

### EXAMPLE 77f

¹H NMR (399.902 MHz, DMSO-d₆) δ 1.20 (4H, d), 1.65 (2H, m), 1.96 (3H, m), 3.17 (2H, m), 3.47 (1H, m), 3.62 (1H, m), 3.74-3.80 (4H, m), 3.97 (1H, m), 4.17 (1H, m), 4.47 (1H, m), 6.68 (1H, s), 7.38 (2H, s), 7.43 (3H, d), 7.71 (3H, d), 7.77 (2H, s), 7.98 (2H, d), 8.09 (2H, d), 8.38 (1H, s), 8.83 (1H, s).

The preparation of phenyl N-[4-[4-[1-(4-cyanophenyl)sulfonylcyclopropyl]-6-[(3S)-3-methylmorpholin-4-yl]pyrimidin-2-yl]phenyl]carbamate is described below.

### Phenyl N-[4-[4-l [-(4-cyanophenyl)sulfonylcyclopropyl]-6-[(3S)-3-methylmorpholin-4-yl]pyrimidin-2-yl]phenyl]carbamate

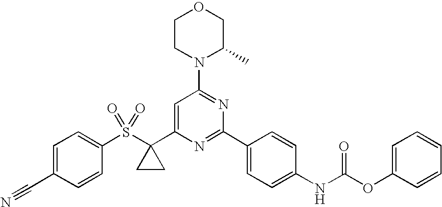

Phenyl chloroformate (0.185 mL, 1.47 mmol) was added dropwise to 4-[1-[2-(4-aminophenyl)-6-[(3S)-3-methylmorpholin-4-yl]pyrimidin-4-yl]cyclopropyl]sulfonylbenzonitrile (700 mg, 1.47 mmol) and sodium bicarbonate (185 mg, 2.21 mmol) in dioxane (20 mL) under nitrogen. The resulting suspension was stirred at RT for 2 hours. The precipitate was collected by filtration, washed with diethyl ether (2 mL) and suspended in water (20 mL) and stirred for 20 minutes. The precipitate was collected by filtration, washed with water (5 mL) then diethyl ether (5 mL) and dried under vacuum to afford the desired material as a white solid (786 mg).

NMR Spectrum: ¹H NMR (399.902 MHz, DMSO-d₆) δ 1.20 (3H, d), 1.67 (2H, m), 1.97 (2H, m), 3.18 (1H, m), 3.45 (1H, m), 3.62 (1H, m), 3.76 (1H, m), 3.97 (1H, m), 4.18 (1H, m), 4.47 (1H, m), 6.71 (1H, s), 7.27 (3H, m), 7.46 (2H, m), 7.53 (2H, d), 7.78 (2H, d), 7.98 (2H, d), 8.09 (2H, d), 10.44 (1H, s).

LCMS Spectrum: m/z (ESI+)(M+H)+=596; HPLC tR=3.01 min.

### 4-[1-[2-(4-Aminophenyl)-6-[(3S)-3-methylmorpholin-4-yl]pyrimidin-4-yl]cyclopropyl]sulfonylbenzonitrile

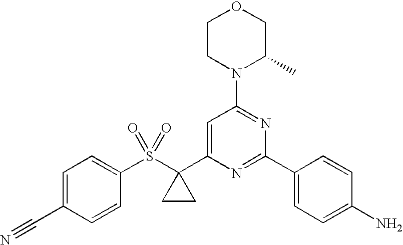

Sodium carbonate (1.693 g, 15.97 mmol) was added to 4-[1-[2-chloro-6-[(3S)-3-methylmorpholin-4-yl]pyrimidin-4-yl]cyclopropyl]sulfonylbenzonitrile (2.23 g, 5.32 mmol) and (4-aminophenyl)boronic acid pinacol ester (1.225 g, 5.59 mmol) in a mixture of DME (40 mL) and water (10.00 mL). The mixture was bubbled with nitrogen for 10 minutes then dichlorobis(triphenylphosphine)palladium(II) (0.374 g, 0.53 mmol) was added and the mixture stirred at 80° C. for 2 hours. The reaction mixture was cooled to RT then diluted with DCM (100 mL) and water (50 mL). The organic layer was washed with saturated brine (50 mL) then dried over MgSO₄, filtered and evaporated to afford crude product. The crude product was purified by flash silica chromatography, elution gradient 30 to 40% ethyl acetate in isohexane followed by 40% ethyl acetate in isohexane, to give the desired material as an orange solid (2.31 g).

NMR Spectrum: ¹H NMR (399.902 MHz, DMSO-d₆) δ 1.18 (3H, d), 1.62 (2H, m), 1.93 (2H, m), 3.14 (1H, m), 3.45 (1H, m), 3.60 (1H, m), 3.74 (1H, m), 3.95 (1H, m), 4.12 (1H, m), 4.42 (1H, m), 5.55 (2H, s), 6.48 (2H, d), 6.56 (1H, s), 7.50 (2H, d), 7.97 (2H, d), 8.08 (2H, d).

LCMS Spectrum: m/z (ESI+)(M+H)+=476; HPLC tR=2.37 min.

### 4-[1-[2-Chloro-6-[(3S)-3-methylmorpholin-4-yl]pyrimidin-4-yl]cyclopropyl]sulfonylbenzonitrile

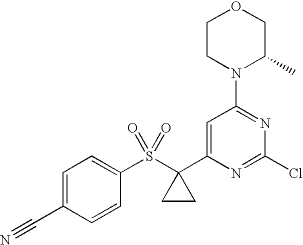

Sodium hydride, 60% dispersion in mineral oil (0.316 g, 7.91 mmol) was added in one portion to 4-[[2-chloro-6-[(3S)-3-methylmorpholin-4-yl]pyrimidin-4-yl]methylsulfonyl]benzonitrile (2.96 g, 7.53 mmol) in DMF (20 mL) at 0° C. under nitrogen. The resulting suspension was stirred for 10 minutes then 1,2-dibromoethane (0.682 mL, 7.91 mmol) was added. The mixture was warmed to 10° C. for 10 minutes then cooled back to 0° C. and a further portion of sodium hydride, 60% dispersion in mineral oil (0.316 g, 7.91 mmol) added. The mixture was warmed to 50° C. and stirred at 50° C. for 2 hours. A further portion of sodium hydride, 60% dispersion in mineral oil (0.158 g, 3.95 mmol) and 1,2-dibromoethane (0.341 ml, 3.95 mmol) was added and stirred for a further 2 hours. The reaction mixture was cooled to RT, quenched with saturated aqueous ammonium chloride (2 mL) and the solvents evaporated. The residues were stirred in water (50 mL) for 15 minutes then the resulting solid collected by filtration. The solid was dissolved in DCM (50 mL), washed with water (20 mL), then saturated brine (20 mL), dried over MgSO₄, filtered and evaporated to give the crude product. The crude product was purified by flash silica chromatography, elution gradient 20 to 40% ethyl acetate in isohexane, to give the desired material as a white solid (2.33 g).

NMR Spectrum: ¹H NMR (399.902 MHz, DMSO-d₆) δ 1.16 (3H, d), 1.60 (2H, m), 1.90 (2H, m), 3.15 (1H, m), 3.40 (1H, m), 3.55 (1H, m), 3.70 (1H, m), 3.90-3.99 (2H, m), 4.27 (1H, m), 6.72 (1H, s), 7.96 (2H, d), 8.11 (2H, d).

LCMS Spectrum: m/z (ESI+)(M+H)+=419; HPLC tR=2.19 min.

### 4-[[2-Chloro-6-[(3S)-3-methylmorpholin-4-yl]pyrimidin-4-yl]methylsulfonyl]benzonitrile

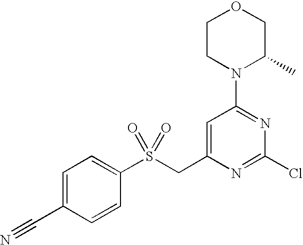

2N Sulfuric acid (0.4 mL) was added to a stirred solution of 4-[[2-chloro-6-[(3S)-3-methylmorpholin-4-yl]pyrimidin-4-yl]methylsulfanyl]benzonitrile (5.18 g, 14.35 mmol) in dioxane (125 mL) and the solution heated to 55° C. Sodium tungstate dihydrate (0.095 g, 0.29 mmol) in water (3.5 mL) was added and the solution was allowed to stir for 5 minutes. Hydrogen peroxide, 30% by wt solution in water (8.80 mL, 86.13 mmol) was added dropwise and the solution stirred at 55° C. for 3 hours. The reaction was cooled to RT then water added until precipitation ceased. The precipitate was collected by filtration, washed with water and dried under vacuum to afford the desired material as a white solid (5.0 g).

NMR Spectrum: ¹H NMR (399.902 MHz, DMSO-d₆) δ 1.18 (3H, d), 3.18 (1H, m), 3.43 (1H, m), 3.58 (1H, m), 3.73 (1H, m), 3.91 (2H, m), 4.18 (1H, m), 4.77 (2H, s), 6.76 (1H, s), 7.99 (2H, d), 8.15 (2H, d).

LCMS Spectrum: m/z (ESI+)(M+H)+=393; HPLC tR=1.99 min.

### 4-[[2-Chloro-6-[(3S)-3-methylmorpholin-4-yl]pyrimidin-4-yl]methylsulfanyl]benzonitrile

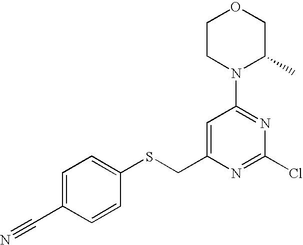

4-Mercaptobenzonitrile (3.48 g, 25.74 mmol) was added to 2-chloro-4-(iodomethyl)-6-[(3S)-3-methylmorpholin-4-yl]pyrimidine (7 g, 19.80 mmol) and DIPEA (5.32 mL, 29.70 mmol) in THF (50 mL). The resulting slurry was stirred at RT for 16 hours then the temperature was increased to 70° C. for a further 16 hours. The reaction mixture was cooled to RT then diluted with DCM (200 mL), and washed sequentially with water (200 mL) then saturated brine (100 mL). The organic layer was dried over MgSO₄, filtered and evaporated to afford crude product. The crude product was purified by flash silica chromatography, elution gradient 10 to 30% ethyl acetate in isohexane, to give the desired material as a yellow solid (5.18 g).

NMR Spectrum: ¹H NMR (399.902 MHz, DMSO-d₆) δ 1.15 (3H, d), 3.16 (1H, m), 3.42 (1H, m), 3.57 (1H, m), 3.71 (1H, m), 3.90-3.99 (2H, m), 4.26 (3H, m), 6.88 (1H, s), 7.55 (2H, d), 7.75 (2H, d).

LCMS Spectrum: m/z (ESI+)(M+H)+=361; HPLC tR=2.33 min.

The preparation of 2-chloro-4-(iodomethyl)-6-[(3S)-3-methylmorpholin-4-yl]pyrimidine was described earlier.

### EXAMPLE 78

### 3-Cyclopropyl-1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]urea

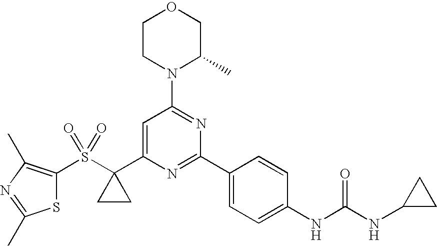

Cyclopropylamine (26 mg, 0.450 mmol) was added to phenyl N-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]carbamate (91 mg, 0.150 mmol) and triethylamine (0.063 mL, 0.450 mmol) in NMP (2 mL). The resulting solution was stirred at 50° C. for 16 hours then cooled to RT and purified by preparative HPLC, eluting with decreasingly polar mixtures of water (containing 1% ammonia) and acetonitrile, to give the desired material as a white solid (64 mg).

NMR Spectrum: ¹H NMR (399.902 MHz, DMSO-d₆) δ 0.42 (2H, m), 0.66 (2H, m), 1.21 (3H, d), 1.62 (2H, m), 1.79 (2H, m), 2.22 (3H, s), 2.56 (1H, m), 2.62 (3H, s), 3.18 (1H, m), 3.48 (1H, m), 3.63 (1H, m), 3.76 (1H, m), 3.97 (1H, m), 4.16 (1H, m), 4.49 (1H, m), 6.43 (1H, d), 6.73 (1H, s), 7.45 (2H, d), 7.93 (2H, d), 8.53 (1H, s).

LCMS Spectrum: m/z (ESI+)(M+H)+=569; HPLC tR=2.27 min.

The compounds below were prepared in an analogous fashion from phenyl N-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 78a | 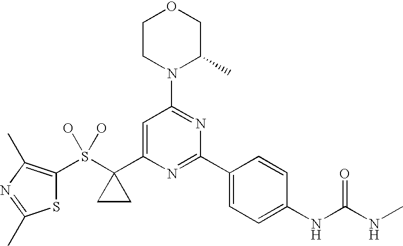 | 1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-methylurea | 543 | 2.08 |
| 78b | 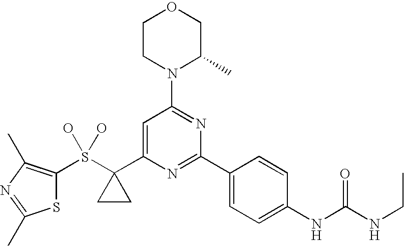 | 3-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-1-ethylurea | 557 | 2.25 |
| 78c | 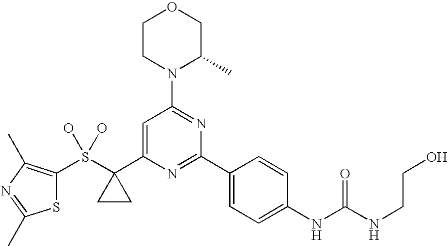 | 1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-(2-hydroxyethyl)urea | 573 | 1.91 |
| 78d | 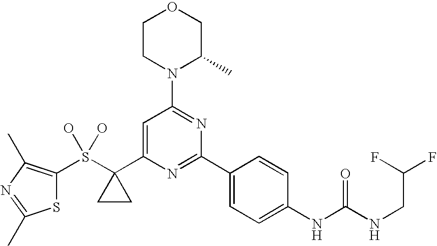 | 3-(2,2-difluoroethyl)-1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]urea | 593 | 2.38 |
| 78e | 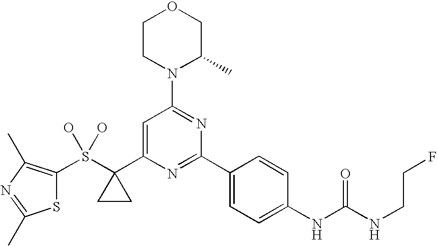 | 1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-(2-fluoroethyl)urea | 575 | 2.24 |
| 78f* | 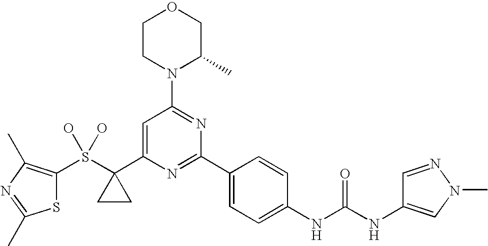 | 1-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]-3-(1-methylpyrazol-4-yl)urea | 609 | 2.13 |

*Stirred for an additional 4 hours at 80° C.

### EXAMPLE 78a

¹H NMR (399.902 MHz, DMSO-d₆) δ 1.21 (3H, d), 1.61 (2H, m), 1.79 (2H, m), 2.22 (3H, s), 2.62 (3H, s), 2.67 (3H, d), 3.18 (1H, m), 3.48 (1H, m), 3.63 (1H, m), 3.76 (1H, m), 3.97 (1H, m), 4.16 (1H, m), 4.49 (1H, m), 6.06 (1H, q), 6.72 (1H, s), 7.44 (2H, d), 7.92 (2H, d), 8.74 (1H, s).

### EXAMPLE 78b

¹H NMR (399.902 MHz, DMSO-d₆) δ 1.07 (3H, t), 1.21 (3H, d), 1.62 (2H, m), 1.79 (2H, m), 2.22 (3H, s), 2.62 (3H, s), 3.10-3.22 (3H, m), 3.48 (1H, m), 3.76 (1H, m), 3.98 (1H, m), 4.16 (1H, m), 4.49 (1H, m), 6.15 (1H, t), 6.72 (1H, s), 7.43 (2H, d), 7.92 (2H, d), 8.66 (1H, s).

### EXAMPLE 78c

¹H NMR (399.902 MHz, DMSO-d₆) δ 1.21 (3H, d), 1.62 (2H, m), 1.79 (2H, m), 2.22 (3H, s), 2.62 (3H, s), 3.14-3.22 (3H, m), 3.44-3.51 (3H, m), 3.63 (1H, m), 3.76 (1H, m), 3.97 (1H, m), 4.16 (1H, m), 4.49 (1H, m), 4.74 (1H, t), 6.24 (1H, t), 6.73 (1H, s), 7.43 (2H, d), 7.93 (2H, d), 8.80 (1H, s).

### EXAMPLE 78d

¹H NMR (399.902 MHz, DMSO-d₆) δ 1.21 (3H, d), 1.62 (2H, m), 1.79 (2H, m), 2.22 (3H, s), 2.62 (3H, s), 3.18 (1H, m), 3.45-3.65 (4H, m), 3.76 (1H, m), 3.97 (1H, m), 4.16 (1H, m), 4.49 (1H, m), 5.93-6.23 (1H, m), 6.53 (1H, t), 6.74 (1H, s), 7.45 (2H, d), 7.95 (2H, d), 8.92 (1H, s).

### EXAMPLE 78e

¹H NMR (399.902 MHz, DMSO-d₆) δ 1.21 (3H, d), 1.62 (2H, m), 1.79 (2H, m), 2.22 (3H, s), 2.62 (3H, s), 3.18 (1H, m), 3.37-3.51 (3H, m), 3.63 (1H, m), 3.77 (1H, m), 3.98 (1H, m), 4.16 (1H, m), 4.41-4.55 (3H, m), 6.43 (1H, t), 6.73 (1H, s), 7.44 (2H, d), 7.93 (2H, d), 8.81 (1H, s).

### EXAMPLE 78f

¹H NMR (399.902 MHz, DMSO-d₆) δ 1.21 (3H, d), 1.62 (2H, m), 1.80 (2H, m), 2.23 (3H, s), 2.63 (3H, s), 3.19 (1H, m), 3.48 (1H, m), 3.64 (1H, m), 3.77 (1H, m), 3.80 (3H, s), 3.98 (1H, m), 4.17 (1H, m), 4.49 (1H, m), 6.74 (1H, s), 7.39 (1H, s), 7.49 (1H,s), 7.49 (2H, d), 7.78 (1H, s), 7.96 (2H, d), 8.38 (1H, s), 8.83 (1H, s).

The preparation of phenyl N-[4-[4-[1-[(2,4-dimethyl-1,3-thiazol-5-yl)sulfonyl]cyclopropyl]-6-[(3S)-3-methylmorpholin-4-yl]pyrimidin-2-yl]phenyl]carbamate is described below.

### Phenyl N-[4-[4-[1-[(2,4-dimethyl-1,3-thiazol-5-yl)sulfonvyl]cyclopropyl]-6-[(3S)-3-methylmorpholin-4-yl]pyrimidin-2-yl]phenyl]carbamate

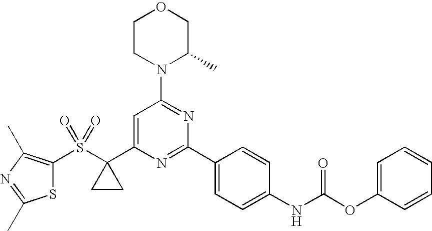

Phenyl chloroformate (0.181 mL, 1.44 mmol) was added dropwise to 4-[4-[1-[(2,4-dimethyl-1,3-thiazol-5-yl)sulfonyl]cyclopropyl]-6-[(3S)-3-methylmorpholin-4-yl]pyrimidin-2-yl]aniline (700 mg, 1.44 mmol) and sodium bicarbonate (182 mg, 2.16 mmol) in dioxane (20 mL) under nitrogen. The resulting suspension was stirred at RT for 2 hours. The precipitate was collected by filtration, washed with diethyl ether (2 mL) and suspended in water (20 mL) and stirred for 20 minutes. The precipitate was collected by filtration, washed with water (5 mL) then diethyl ether (5 mL) and dried under vacuum to afford the desired material as a beige solid (637 mg).

NMR Spectrum: ¹H NMR (399.902 MHz, DMSO-d₆) δ 1.22 (3H, d), 1.63 (2H, m), 1.81 (2H, m), 2.24 (3H, s), 2.61 (3H, s), 3.20 (1H, m), 3.49 (1H, m), 3.63 (1H, m), 3.77 (1H, m), 3.98 (1H, m), 4.17 (1H, m), 4.51 (1H, m), 6.78 (1H, s), 7.28 (3H, m), 7.46 (2H, m), 7.58 (2H, d), 8.02 (2H, d), 10.43 (1H, s).

LCMS Spectrum: m/z (ESI+)(M+H)+=606; HPLC tR=2.91 min.

### 4-[4-[1-[(2,4-Dimethyl-1,3-thiazol-5-yl)sulfonyl]cyclopropyl]-6-[(3S)-3-methylmorpholin-4-yl]pyrimidin-2-yl]aniline

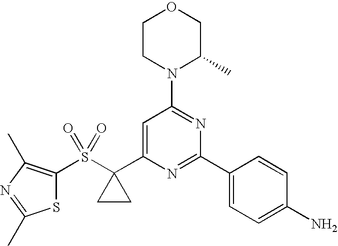

Sodium carbonate (2.105 g, 19.86 mmol) was added to 2-chloro-4-[1-[(2,4-dimethyl-1,3-thiazol-5-yl)sulfonyl]cyclopropyl]-6-[(3S)-3-methylmorpholin-4-yl]pyrimidine (2.84 g, 6.62 mmol) and (4-aminophenyl)boronic acid pinacol ester (1.523 g, 6.95 mmol) in a mixture of DME (40 mL) and water (10 mL). The mixture was bubbled with nitrogen for 10 minutes then dichlorobis(triphenylphosphine)palladium(II) (0.465 g, 0.66 mmol) was added and the mixture stirred at 80° C. for 2 hours. Further dichlorobis(triphenylphosphine)palladium(II) (0.232 g, 0.33 mmol) was added and the mixture was stirred at 80° C. for a further 4 hours. The reaction mixture was cooled to RT then diluted with DCM (250 mL) and water (100 mL). The organic layer was dried over MgSO₄, filtered and evaporated to afford crude product. The crude product was purified by flash silica chromatography, elution gradient 40 to 50% ethyl acetate in isohexane followed by 50% ethyl acetate in isohexane, to give a material which was further purified by ion exchange chromatography using an SCX column, eluting with 2M ammonia in methanol, to give the desired material as an orange solid (2.59 g).

NMR Spectrum: ¹H NMR (399.902 MHz, DMSO-d₆) δ 1.19 (3H, d), 1.59 (2H, m), 1.77 (2H, m), 2.22 (3H, s), 2.62 (3H, s), 3.15 (1H, m), 3.47 (1H, m), 3.61 (1H, m), 3.75 (1H, m), 3.96 (1H, m), 4.12 (1H, m), 4.45 (1H, m), 5.54 (2H, s), 6.54 (2H, d), 6.62 (1H, s), 7.76 (2H, d).

LCMS Spectrum: m/z (ESI+)(M+H)+=486; HPLC tR=2.17 min.

### 2-Chloro-4-[1-[(2,4-dimethyl-1,3-thiazol-5-yl)sulfonyl]cyclopropyl]-6-[(3S)-3-methylmorpholin-4-yl]pyrimidine

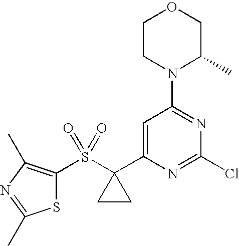

Sodium hydroxide (15.94 g, 398.59 mmol) in water (16 mL) was added to a stirred solution of 2-chloro-4-[(2,4-dimethyl-1,3-thiazol-5-yl)sulfonylmethyl]-6-[(3S)-3-methylmorpholin-4-yl]pyrimidine (2.92 g, 7.25 mmol), 1,2-dibromoethane (1.874 mL, 21.74 mmol) and tetrabutylammonium bromide (0.234 g, 0.72 mmol) in toluene (100 mL). The resulting solution was stirred at RT for 90 minutes then at 60° C. for 1 hour. The reaction mixture was diluted with water (300 mL), the organic layer separated and washed with saturated brine (100 mL) then dried over MgSO₄, filtered and evaporated to afford crude product. The crude product was purified by flash silica chromatography, elution gradient 30 to 50% ethyl acetate in isohexane, to give the desired material as a white solid (2.84 g).

NMR Spectrum: ¹H NMR (399.902 MHz, DMSO-d₆) δ 1.18 (3H, d), 1.59 (2H, m), 1.76 (2H, m), 2.29 (3H, s), 2.68 (3H, s), 3.17 (1H, m), 3.42 (1H, m), 3.56 (1H, m), 3.72 (1H, m), 3.91-4.00 (2H, m), 4.33 (1H, m), 6.82 (1H, s).

LCMS Spectrum: m/z (ESI+)(M+H)+=429; HPLC tR=2.09 min.

### 2-Chloro-4-[(2,4-dimethyl-1,3-thiazol-5-yl)sulfonylmethyl]-6-[(3S)-3-methylmorpholin-4-yl]pyrimidine

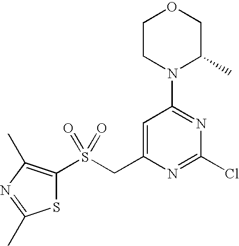

Sodium 2,4-dimethylthiazole-5-sulfinate (5.2 g, 26.10 mmol) was added to 2-chloro-4-(iodomethyl)-6-[(3S)-3-methylmorpholin-4-yl]pyrimidine (4.01 g, 11.35 mmol) in DMF (50 mL). The resulting mixture was stirred at RT for 1 hour. The reaction mixture was evaporated to dryness and redissolved in DCM (250 mL), and washed sequentially with water (150 mL) and saturated brine (100 mL). The organic layer was dried over MgSO₄, filtered and evaporated to afford crude product. The crude product was purified by flash silica chromatography, elution gradient 20 to 50% ethyl acetate in isohexane followed by 50% ethyl acetate in isohexane, to give the desired material as a yellow solid (4.77 g).

NMR Spectrum: ¹H NMR (399.902 MHz, DMSO-d₆) δ 1.19 (3H, d), 2.40 (3H, s), 2.67 (3H, s), 3.19 (1H, m), 3.44 (1H, m), 3.59 (1H, m), 3.73 (1H, m), 3.93 (2H, m), 4.23 (1H, m), 4.69 (2H, s), 6.83 (1H, s).

LCMS Spectrum: m/z (ESI+)(M+H)+=403; HPLC tR=1.82 min.

### Sodium 2,4-dimethylthiazole-5-sulfinate

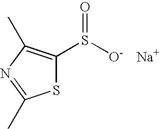

A solution of sodium sulfite (2.98 g, 23.62 mmol) in water (25 mL) was stirred at RT for 10 minutes. Sodium bicarbonate (3.97 g, 47.24 mmol) was added and the resulting solution was stirred at 50° C. for 1 hour. 2,4-Dimethylthiazole-5-sulfonyl chloride (5 g, 23.62 mmol) was added portion wise to the solution and stirring was continued at 50° C. for 18 hours. The reaction mixture was evaporated to dryness and redissolved in methanol (75 mL). The suspension was allowed to stir at RT for 20 minutes. The suspension was filtered and the filtrate evaporated to afford the desired material as a yellow solid (5.21 g), which was dried under vacuum and used without further purification.

NMR Spectrum: ¹H NMR (399.902 MHz, DMSO-d₆) δ 2.28 (3H, s).

The preparation of 2-chloro-4-(iodomethyl)-6-[(3S)-3-methylmorpholin-4-yl]pyrimidine was described earlier.

### EXAMPLE 79

### 3-Cyclopropyl-1-[4-[4-[1-(3-hydroxy-3-methylbutyl)sulfonylcyclopropyl]-6-[(3S)-3-methylmorpholin-4-yl]pyrimidin-2-yl]phenyl]urea

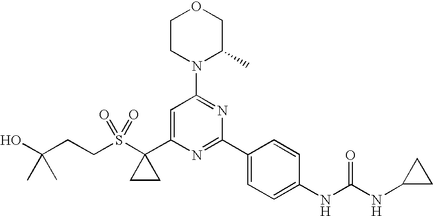

Cyclopropylamine (33 mg, 0.573 mmol) was added to phenyl N-[4-[4-[1-(3-hydroxy-3-methylbutyl)sulfonylcyclopropyl]-6-[(3S)-3-methylmorpholin-4-yl]pyrimidin-2-yl]phenyl]carbamate (111 mg, 0.191 mmol) and triethylamine (0.080 mL, 0.573 mmol) in NMP (2 mL). The resulting solution was stirred at 50° C. for 16 hours then cooled to RT and purified by preparative HPLC, eluting with decreasingly polar mixtures of water (containing 1% ammonia) and acetonitrile, to give the desired material as a white solid (83 mg).

NMR Spectrum: ¹H NMR (399.902 MHz, DMSO-d₆) δ 0.43 (2H, m), 0.65 (2H, m), 1.12 (6H, s), 1.24 (3H, d), 1.56 (2H, m), 1.65 (2H, m), 1.85 (2H, m), 2.56 (1H, m), 3.21 (1H, m), 3.49 (1H, m), 3.56 (2H, m), 3.63 (1H, m), 3.77 (1H, m), 3.98 (1H, m), 4.22 (1H, m), 4.49 (1H, s), 4.56 (1H, m), 6.44 (1H, d), 6.76 (1H, s), 7.50 (2H, d), 8.24 (2H, d), 8.57 (1H, s),

LCMS Spectrum: m/z (ESI+)(M+H)+=544; HPLC tR=2.10 min.

The compounds below were prepared in an analogous fashion from phenyl N-[4-[4-[1-(3-hydroxy-3-methylbutyl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 79a | 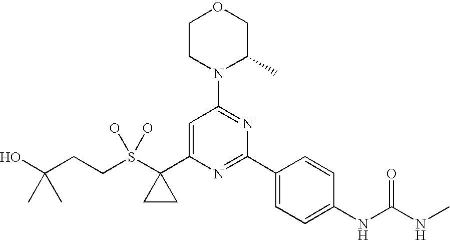 | 1-[4-[4-[1-(3-hydroxy-3-methylbutyl)sulfonylcyclopropyl]-6-[(3S)-3-methylmorpholin-4-yl]pyrimidin-2-yl]phenyl]-3-methylurea | 518 | 1.93 |
| 79b | 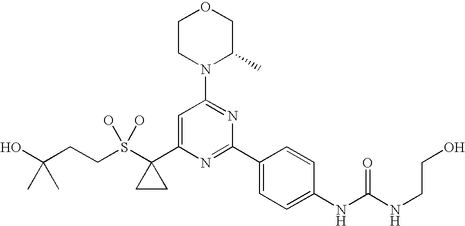 | 3-(2-hydroxyethyl)-1-[4-[4-[1-(3-hydroxy-3-methylbutyl)sulfonylcyclopropyl]-6-[(3S)-3-methylmorpholin-4-yl]pyrimidin-2-yl]phenyl]urea | 548 | 1.78 |
| 79c | 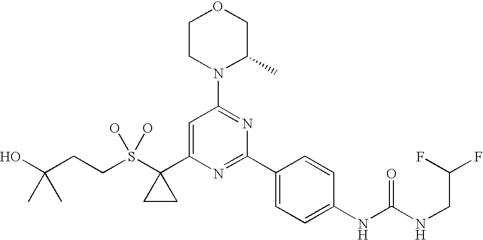 | 3-(2,2-difluoroethyl)-1-[4-[4-[1-(3-hydroxy-3-methylbutyl)sulfonylcyclopropyl]-6-[(3S)-3-methylmorpholin-4-yl]pyrimidin-2-yl]phenyl]urea | 568 | 2.18 |
| 79d | 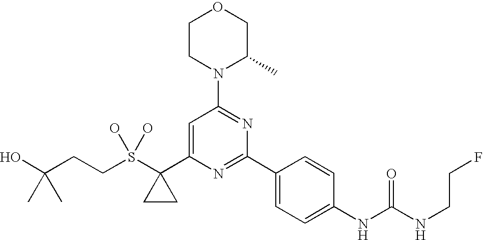 | 3-(2-fluoroethyl)-1-[4-[4-[1-(3-hydroxy-3-methylbutyl)sulfonylcyclopropyl]-6-[(3S)-3-methylmorpholin-4-yl]pyrimidin-2-yl]phenyl]urea | 550 | 2.06 |
| 79e* | 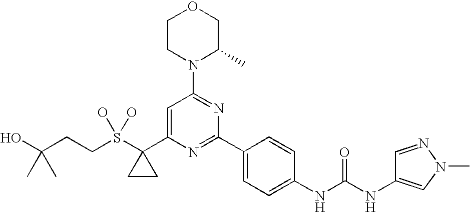 | 1-[4-[4-[1-(3-hydroxy-3-methylbutyl)sulfonylcyclopropyl]-6-[(3S)-3-methylmorpholin-4-yl]pyrimidin-2-yl]phenyl]-3-(1-methylpyrazol-4-yl)urea | 584 | 1.98 |
| 79f | 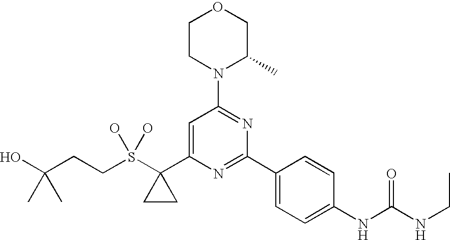 | 1-ethyl-3-[4-[4-[1-(3-hydroxy-3-methylbutyl)sulfonylcyclopropyl]-6-[(3S)-3-methylmorpholin-4-yl]pyrimidin-2-yl]phenyl]urea | 532 | 2.07 |

*6 Equivalents of triethylamine were used.

### EXAMPLE 79a

¹H NMR (399.902 MHz, DMSO-d₆) δ 1.13 (6H, s), 1.23 (3H, d), 1.55 (2H, m), 1.65 (2H, m), 1.85 (2H, m), 2.67 (3H, d), 3.21 (1H, m), 3.45-3.58 (3H, m), 3.63 (1H, m), 3.77 (1H, m), 3.98 (1H, m), 4.22 (1H, m), 4.49 (1H, s), 4.57 (1H, m), 6.07 (1H, m), 6.75 (1H, s), 7.49 (2H, d), 8.23 (2H, d), 8.77 (1H, s).

### EXAMPLE 79b

¹H NMR (399.902 MHz, DMSO-d₆) δ 1.13 (6H, s), 1.24 (3H, d), 1.55 (2H, m), 1.65 (2H, m), 1.85 (2H, m), 3.16-3.24 (3H, m), 3.45-3.49 (3H, m), 3.56 (2H, m), 3.63 (1H, m), 3.77 (1H, m), 3.98 (1H, m), 4.22 (1H, m), 4.49 (1H, s), 4.56 (1H, m), 4.74 (1H, t), 6.25 (1H, t), 6.76 (1H, s), 7.48 (2H, d), 8.23 (2H, d), 8.82 (1H, s).

### EXAMPLE 79c

¹H NMR (399.902 MHz, DMSO-d₆) δ 1.13 (6H, s), 1.24 (3H, d), 1.56 (2H, m), 1.65 (2H, m), 1.85 (2H, m), 3.21 (1H, m), 3.45-3.65 (6H, m), 3.77 (1H, m), 3.98 (1H, m), 4.23 (1H, m), 4.49 (1H, s), 4.57 (1H, m), 5.93-6.23 (1H, m), 6.53 (1H, t), 6.77 (1H, s), 7.50 (2H, d), 8.26 (2H, d), 8.95 (1H, s).

### EXAMPLE 79d

¹H NMR (399.902 MHz, DMSO-d₆) δ 1.12 (6H, s), 1.24 (3H, d), 1.55 (2H, m), 1.65 (2H, m), 1.85 (2H, m), 3.21 (1H, m), 3.36-3.58 (5H, m), 3.64 (1H, m), 3.77 (1H, m), 3.98 (1H, m), 4.22 (1H, m), 4.41-4.58 (4H, m), 6.43 (1H, t), 6.76 (1H, 7.49 (2H, d), 8.25 (2H, d), 8.83 (1H, s).

### EXAMPLE 79e

¹H NMR (399.902 MHz, DMSO-d₆) δ 1.13 (6H, s), 1.24 (3H, d), 1.56 (2H, m), 1.66 (2H, m), 1.86 (2H, m), 3.22 (1H, m), 3.49 (1H, m), 3.57 (2H, m), 3.64 (1H, m), 3.78 (4H, m), 3.98 (1H, m), 4.23 (1H, m), 4.50 (1H, s), 4.57 (1H, m), 6.77 (1H, s, 7.39 (1H, s), ) 7.54 (2H, d), 7.78 (1H, s), 8.27 (2H, d), 8.38 (1H, s), 8.86 (1H, s)

### EXAMPLE 79f

¹H NMR (399.902 MHz, DMSO-d₆) δ 1.07 (3H, t), 1.12 (6H, s), 1.23 (3H, d), 1.59 (4H, m), 1.84 (2H, m), 3.17 (3H, m), 3.54 (4H, m), 3.76 (1H, m), 3.98 (1H, m), 4.23 (1H, m), 4.56 (2H, m), 6.17 (1H, m), 6.75 (1H, s), 7.48 (2H, d), 8.23 (2H, d), 8.72 (1H, s)

The preparation of phenyl N-[4-[4-[1-(3-hydroxy-3-methylbutyl)sulfonylcyclopropyl]-6-[(3S)-3-methylmorpholin-4-yl]pyrimidin-2-yl]phenyl]carbamate is described below.

### Phenyl N-[4-[4-[1-(3-hydroxy-3-methylbutyl)sulfonylcyclopropyl]-6-[(3S)-3-methylmorpholin-4-yl]pyrimidin-2-yl]phenyl]carbamate

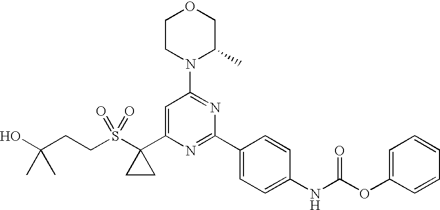

Phenyl chloroformate (0.191 mL, 1.52 mmol) was added dropwise to 4-[1-[2-(4-aminophenyl)-6-[(3S)-3-methylmorpholin-4-yl]pyrimidin-4-yl]cyclopropyl]sulfonyl-2-methylbutan-2-ol (700 mg, 1.52 mmol) and sodium bicarbonate (192 mg, 2.28 mmol) in dioxane (20 mL) under nitrogen. The resulting suspension was stirred at RT for 2 hours. The precipitate was collected by filtration then washed with diethyl ether (2 mL). The precipitate was suspended in water (20 mL) then extracted into DCM (50 mL). The organic layer was dried over MgSO₄, filtered and evaporated to afford the desired material as a yellow solid (774 mg).

NMR Spectrum: ¹H NMR (399.902 MHz, DMSO-d₆) δ 1.12 (6H, s), 1.24 (3H, d), 1.57 (2H, m), 1.66 (2H, m), 1.85 (2H, m), 3.22 (1H, m), 3.46-3.57 (3H, m), 3.64 (1H, m), 3.77 (1H, m), 3.98 (1H, m), 4.24 (1H, m), 4.58 (1H, m), 6.80 (1H, s), 7.28 (3H, m), 7.46 (2H, m), 7.62 (2H, d), 8.33 (2H, d), 10.43 (1H, s).

LCMS Spectrum: m/z (ESI+)(M+H)+=581; HPLC tR=2.63 min.

### 4-[1-[2-(4-Aminophenyl)-6-[(3S)-3-methylmorpholin-4-yl]pyrimidin-4-yl]cyclopropyl]sulfonyl-2-methylbutan-2-ol

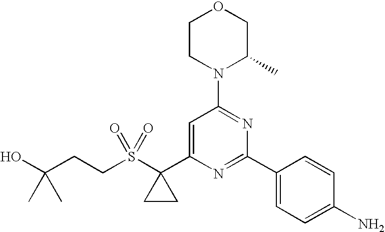

TFA (5 mL) was added to tert-butyl N-[4-[4-[1-(3-hydroxy-3-methylbutyl)sulfonylcyclopropyl]-6-[(3S)-3-methylmorpholin-4-yl]pyrimidin-2-yl]phenyl]carbamate (2.08 g, 3.71 mmol) in DCM (5 mL). The resulting solution was stirred at RT for 1 hour then added to an SCX column. The crude product was eluted from the column using 2M ammonia in methanol the further purified by flash silica chromatography, elution gradient 40 to 50% ethyl acetate in isohexane, to give the desired material as a white solid (1.15 g).

NMR Spectrum: ¹H NMR (399.902 MHz, DMSO-d₆) δ 1.13 (6H, s), 1.22 (3H, d), 1.52 (2H, m), 1.63 (2H, m), 1.85 (2H, m), 3.18 (1H, m), 3.48 (1H, m), 3.55 (2H, m), 3.62 (1H, m), 3.76 (1H, m), 3.97 (1H, m), 4.19 (1H, m), 4.48 (1H, s), 4.53 (1H, m), 5.57 (2H, s), 6.59 (2H, d), 6.65 (1H, s), 8.07 (2H, d).

LCMS Spectrum: m/z (ESI+)(M+H)+=461; HPLC tR=1.96 min.

### tert-Butyl N-[4-[4-[1-(3-hydroxy-3-methylbutyl)sulfonylcyclopropyl]-6-[(3S)-3-methylmorpholin-4-yl]pyrimidin-2-yl]phenyl]carbamate

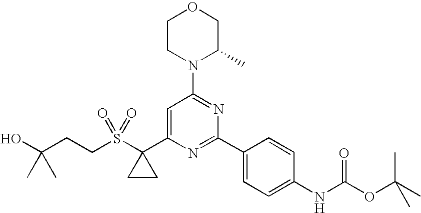

Sodium carbonate (1.299 g, 12.25 mmol) was added to 4-[1-[2-chloro-6-[(3S)-3-methylmorpholin-4-yl]pyrimidin-4-yl]cyclopropyl]sulfonyl-2-methylbutan-2-ol (1.65 g, 4.08 mmol) and (4-boc-aminophenyl)boronic acid pinacol ester (1.369 g, 4.29 mmol) in a mixture of DME (40 mL) and water (10 mL). The mixture was bubbled with nitrogen for 10 minutes then 1,1′-bis(diphenylphosphino)ferrocenedichloropalladium(II) (0.296 g, 0.41 mmol) was added and the mixture stirred at 80° C. for 8 hours. The reaction mixture was cooled to RT then diluted with DCM (150 mL), and washed with water (100 mL). The organic layer was dried over MgSO₄, filtered and evaporated to afford crude product. The crude product was purified by flash silica chromatography, elution gradient 30 to 50% ethyl acetate in isohexane, to give the desired material as a yellow gum (2.08 g).

LCMS Spectrum: m/z (ESI+)(M+H)+=561; HPLC tR=2.69 min.

### 4-[1-[2-Chloro-6-[(3S)-3-methylmorpholin-4-yl]pyrimidin-4-yl]cyclopropyl]sulfonyl-2-methylbutan-2-ol

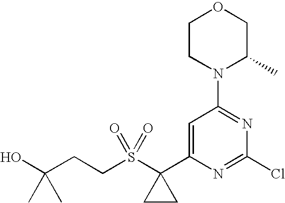

Sodium hydroxide (11.93 g, 298.37 mmol) in water (12 mL) was added to a stirred solution of 4-[[2-chloro-6-[(3S)-3-methylmorpholin-4-yl]pyrimidin-4-yl]methylsulfonyl]-2-methylbutan-2-ol (2.05 g, 5.42 mmol), 1,2-dibromoethane (1.402 mL, 16.27 mmol) and tetrabutylammonium bromide (0.175 g, 0.54 mmol) in toluene (75 mL). The resulting solution was stirred at RT for 5 hours then at 45° C. for 3 hours. The reaction mixture was diluted with water (100 mL), the organic layer separated and dried over MgSO₄, filtered and evaporated to afford crude product. The crude product was purified by flash silica chromatography, elution gradient 30 to 50% ethyl acetate in isohexane, to give the desired material as a yellow dry film (1.75 g).

NMR Spectrum: ¹H NMR (399.902 MHz, DMSO-d₆) δ 1.13 (6H, s), 1.22 (3H, d), 1.52 (2H, m), 1.61 (2H, m), 1.80 (2H, m), 3.21 (1H, m), 3.36-3.47 (3H, m), 3.58 (1H, m), 3.72 (1H, m), 3.93 (1H, m), 4.05 (1H, m), 4.40 (1H, m), 4.46 (1H, s), 6.94 (1H, s).

LCMS Spectrum: m/z (ESI+)(M+H)+=404; HPLC tR=1.83 min.

### 4-[[2-Chloro-6-[(3S)-3-methylmorpholin-4-yl]pyrimidin-4-yl]methylsulfonyl]-2-methylbutan-2-ol

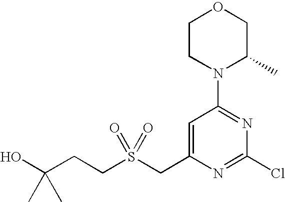

2N Sulfuric Acid (0.307 mL) was added to a stirred solution of 4-[[2-chloro-6-[(3S)-3-methylmorpholin-4-yl]pyrimidin-4-yl]methylsulfanyl]-2-methylbutan-2-ol (3.78 g, 10.93 mmol) in dioxane (100 mL) and the solution heated to 55° C. Sodium tungstate dihydrate (0.072 g, 0.22 mmol) in water (3 mL) was added and the solution was allowed to stir for 5 minutes. Hydrogen peroxide, 30% by wt solution in water (6.7 mL, 65.57 mmol) was added dropwise and the solution stirred at 55° C. for 2.5 hours. The reaction mixture was cooled to RT and diluted with water (100 mL), and extracted with DCM (2×200 mL). The organic layer was washed with brine (50 mL) then dried over MgSO₄, filtered and evaporated to afford the desired material as a cream solid (4.39 g).

NMR Spectrum: ¹H NMR (399.902 MHz, DMSO-d₆) δ 1.15 (6H, s), 1.23 (3H, d), 1.82 (2H, m), 3.19-3.30 (3H, m), 3.46 (1H, m), 3.61 (1H, m), 3.74 (1H, m), 3.93-4.02 (2H, m), 4.31 (1H, m), 4.46 (2H, s), 4.48 (1H, s), 6.95 (1H, s).

LCMS Spectrum: m/z (ESI+)(M+H)+=378; HPLC tR=1.64 min.

### 4-[[2-Chloro-6-[(3S)-3-methylmorpholin-4-yl]pyrimidin-4-yl]methylsulfanyl]-2-methylbutan-2-ol

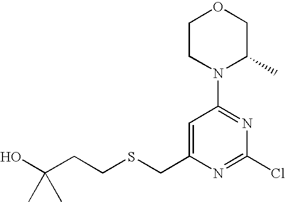

Methylmagnesium bromide, 3M in diethyl ether (13.17 mL, 39.50 mmol) was added dropwise to methyl 3-[[2-chloro-6-[(3S)-3-methylmorpholin-4-yl]pyrimidin-4-yl]methylsulfanyl]propanoate (4.14 g, 11.97 mmol) in THF (50 mL) at 0° C. over a period of 10 minutes under nitrogen. The resulting solution was stirred at 0° C. for 30 minutes then at RT for 90 minutes. The reaction mixture was quenched with saturated aqueous ammonium chloride solution (1 mL), diluted with water (100 mL) then extracted with DCM (3×100 mL), the organic layer was washed with brine (100 mL) then dried over MgSO₄, filtered and evaporated to afford the desired material as a yellow gum (3.96 g).

NMR Spectrum: ¹H NMR (399.902 MHz, DMSO-d₆) δ 1.08 (6H, s), 1.21 (3H, d), 1.61 (2H, m), 2.54 (2H, m), 3.18 (1H, m), 3.44 (1H, m), 3.57 (2H, s), 3.61 (1H, m), 3.72 (1H, m), 3.91-4.01 (2H, m), 4.23 (1H, s), 4.32 (1H, m), 6.78 (1H, s).

LCMS Spectrum: m/z (ESI+)(M+H)+=346; HPLC tR=1.79 min.

### Methyl 3-[[2-chloro-6-[(3S)-3-methylmorpholin-4-yl]pyrimidin-4-yl]methylsulfanyl]propanoate

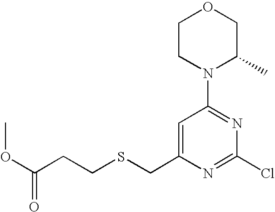

Methyl 3-mercaptopropionate (2.55 g, 21.21 mmol) was added to 2-chloro-4-(iodomethyl)-6-[(3S)-3-methylmorpholin-4-yl]pyrimidine (5 g, 14.14 mmol) and DIPEA (3.80 mL, 21.21 mmol) in THF (50 mL). The resulting slurry was stirred at RT for 16 hours. The reaction mixture was diluted with DCM (150 mL), and washed sequentially with water (100 mL) then saturated brine (50 mL). The organic layer was dried over MgSO₄, filtered and evaporated to afford crude product. The crude product was purified by flash silica chromatography, elution gradient 10 to 30% ethyl acetate in isohexane, to give the desired material as a colourless liquid (4.6 g).

NMR Spectrum: ¹H NMR (399.902 MHz, DMSO-d₆) δ 1.21 (3H, d), 2.65 (2H, t), 2.76 (2H, t), 3.19 (1H, m), 3.45 (1H, m), 3.56-3.64 (6H, m), 3.72 (1H, m), 3.91-4.00 (2H, m), 4.33 (1H, m), 6.81 (1H, s).

LCMS Spectrum: m/z (ESI+)(M+H)+=346; HPLC tR=2.05 min.

The preparation of 2-chloro-4-(iodomethyl)-6-[(3S)-3-methylmorpholin-4-yl]pyrimidine was described earlier.

### EXAMPLE 80

3-(2,2-Difluoroethyl)-1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]urea

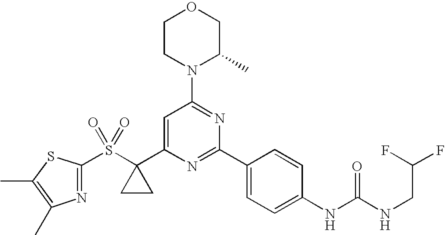

Phenyl N-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]carbamate (150 mg, 0.25 mmol), triethylamine (0.104 mL, 0.74 mmol) and 2,2-difluoroethanamine (60.2 mg, 0.74 mmol) were added to dioxane (10 mL) and heated at 50° C. for 72 hours. The reaction mixture was evaporated under reduced pressure to a gum which was purified by preparative HPLC, eluting with decreasingly polar mixtures of water (containing 1% ammonia) and acetonitrile, to give the desired material as a white solid (83 mg).

NMR Spectrum: ¹H NMR (400.132 MHz, DMSO-d₆) δ 1.21 (3H, d), 1.77-1.72 (2H, m), 1.95-1.88 (2H, m), 2.37 (3H, s), 2.40 (3H, s), 3.23-3.13 (1H, m), 3.66-3.41 (4H, m), 3.80-3.73 (1H, m), 3.97 (1H, d), 4.17 (1H, d), 4.51-4.41 (1H, m), 6.23-5.91 (1H, m), 6.51 (1H, t), 6.79 (1H, s), 7.44 (2H, d), 7.90 (2H, d), 8.91 (1H, s)

LCMS Spectrum: m/z (ESI+)(M+H)+=493; HPLC tR=2.42 min.

The compounds below were prepared in an analogous fashion from phenyl N-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 80a | 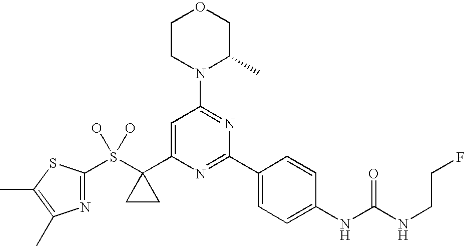 | 1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-(2-fluoroethyl)urea | 576 | 2.25 |
| 80b | 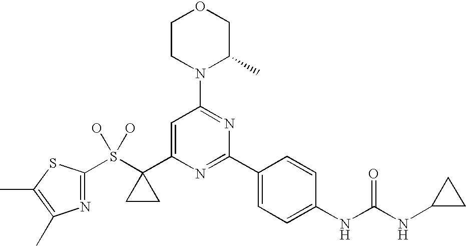 | 3-cyclopropyl-1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]urea | 569 | 2.23 |
| 80c | 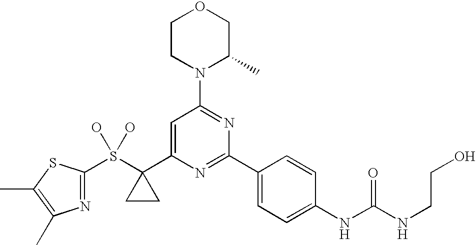 | 1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-(2-hydroxyethyl)urea | 573 | 1.90 |
| 80d* | 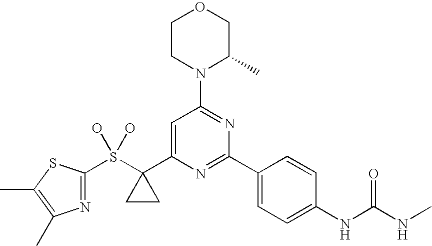 | 1-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-3-methylurea | 543 | 2.06 |
| 80e* | 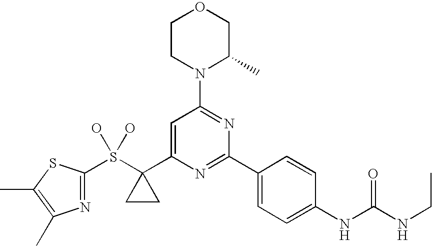 | 3-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]-1-ethylurea | 557 | 2.21 |

*Stirred at RT for 16 hours

### EXAMPLE 80a

¹H NMR (400.132 MHz, DMSO-d₆) δ 1.21 (3H, d), 1.78-1.71 (2H, m), 1.94-1.89 (2H, m), 2.37 (3H, s), 2.40 (3H, s), 3.24-3.13 (1H, m), 3.51-3.35 (4H, m), 3.65-3.59 (1H, m), 3.79-3.74 (1H, m), 4.01-3.94 (1H, m), 4.17 (1H, d), 4.56-4.38 (3H, m), 6.41 (1H, t), 7.43 (2H, d), 7.89 (2H, d), 8.80 (1H, s)

### EXAMPLE 80b

¹H NMR (400.132 MHz, DMSO-d₆) δ 0.45-0.38 (2H, m), 0.69-0.61 (2H, m), 1.21 (3H, d), 1.76-1.72 (2H, m), 1.94-1.89 (2H, m), 2.37 (3H, s), 2.41 (3H, s), 2.59-2.53 (1H, m), 3.24-3.13 (1H, m), 3.47 (1H, t), 3.64-3.59 (1H, m), 3.80-3.72 (1H, m), 3.98 (1H, d), 4.17 (1H, d), 4.50-4.41 (1H, m), 6.41 (1H, d), 6.78 (1H, s), 7.43 (2H, d), 7.88 (2H, d), 8.53 (1H, s)

### EXAMPLE 80c

¹H NMR (400.132 MHz, DMSO-d₆) δ 1.21 (3H, d), 1.77-1.72 (2H, m), 1.94-1.89 (2H, m), 2.37 (3H, s), 2.40 (4H, s), 3.51-3.42 (3H, m), 3.65-3.59 (1H, m), 3.79-3.74 (1H, m), 3.97 (1H, d), 4.06 (2H, q), 4.17 (1H, d), 4.50-4.41 (1H, m), 4.72 (1H, t), 6.23 (1H, t), 6.78 (1H, s), 7.41 (2H, d), 7.88 (2H, d), 8.79 (1H, s)

### EXAMPLE 80d

¹H NMR (400.132 MHz, DMSO-d₆) δ 1.21 (3H, d), 1.78-1.72 (2H, m), 1.94-1.89 (2H, m), 2.37 (3H, s), 2.40 (3H, s), 2.66 (3H, d), 3.23-3.13 (1H, m), 3.52-3.42 (1H, m), 3.65-3.59 (1H, m), 3.79-3.74 (1H, m), 3.97 (1H, d), 4.20-4.13 (1H, m), 4.49-4.41 (1H, m), 6.07-6.03 (1H, m), 6.78 (1H, s), 7.43 (2H, d), 7.87 (2H, d), 8.73 (1H, s)

### EXAMPLE 80e

¹H NMR (400.132 MHz, DMSO-d₆) 1.07 (3H, t), 1.21 (3H, d), 1.77-1.71 (2H, m), 1.95-1.88 (2H, m), 2.37 (3H, s), 2.41 (3H, s), 3.23-3.07 (3H, m), 3.47 (1H, t), 3.65-3.59 (1H, m), 3.79-3.73 (1H, m), 3.98 (1H, d), 4.16 (1H, d), 4.51-4.40 (1H, m), 6.14 (1H, t), 6.78 (1H, s), 7.42 (2H, d), 7.88 (2H, d), 8.65 (1H, s)

The preparation of phenyl N-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]carbamate is described below.

### Phenyl N-[4-[4-[1-[(4,5-dimethyl-1,3-thiazol-2-yl)sulfonvyl]cyclopropyl]-6-[(3-methylmorpholin-4-yl]pyrimidin-2-yl]phenyl]carbamate

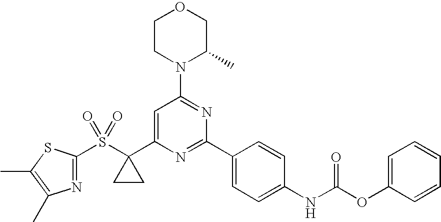

Phenyl chloroformate (0.866 mL, 6.91 mmol) was added dropwise to 4-[4-[1-[(4,5-dimethyl-1,3-thiazol-2-yl)sulfonyl]cyclopropyl]-6-[(3S)-3-methylmorpholin-4-yl]pyrimidin-2-yl]aniline (2.58 g, 5.31 mmol) and sodium hydrogencarbonate (8.93 g, 106.26 mmol) in DCM (52.3 mL) at RT under nitrogen. The resulting suspension was stirred at RT for 90 minutes, saturated ammonium chloride solution added followed by DCM (40 mL). The organics were separated, washed with water (50 mL) and saturated brine (50 mL), dried over MgSO₄ and evaporated to give the desired material (2.91 g).

NMR Spectrum: ¹H NMR (400.132 MHz, CDCl₃) δ 1.34 (3H, d), 1.87-1.77 (2H, m), 2.14-2.10 (2H, m), 2.34 (3H, s), 2.36 (3H, s), 3.32 (1H, t), 3.60 (1H, t), 3.77-3.72 (1H, m), 3.85-3.81 (1H, m), 4.04 (1H, d), 4.20 (1H, d), 4.53-4.44 (1H, m), 7.04 (1H, s), 7.10 (1H, s), 7.28-7.17 (3H, m), 7.40 (2H, t), 7.46 (2H, d), 8.16 (2H, d)

LCMS Spectrum: m/z (ESI+)(M+H)+=606; HPLC tR=2.90 min.

### 4-[4-[1-[(4,5-Dimethyl-1,3-thiazol-2-yl)sulfonyl]cyclopropyl]-6-[(3S)-3-methylmorpholin-4-yl]pyrimidin-2-yl]aniline

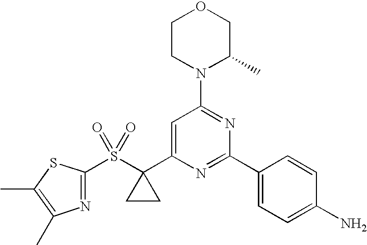

Dichlorobis(triphenylphosphine)palladium(II) (0.219 g, 0.31 mmol) was added in one portion to 2-chloro-4-[1-[(4,5-dimethyl-1,3-thiazol-2-yl)sulfonyl]cyclopropyl]-6-[(3S)-3-methylmorpholin-4-yl]pyrimidine (2.68 g, 6.25 mmol), 4-(4,4,5,5-tetramethyl-1,3,2-dioxaborolan-2-yl)aniline (1.369 g, 6.25 mmol) and sodium bicarbonate (15.62 mL, 31.24 mmol) in a mix of solvents (18% DMF, 82% of a 7:3:2 mixture of DME:water:ethanol) (75 mL) and the resulting mixture stirred at 80° C. for 16 hours under an inert atmosphere. The reaction mixture was diluted with ethyl acetate (200 mL), and washed sequentially with water (2×150 mL) and saturated brine (3×100 mL). The organic layer was dried over MgSO₄, filtered and evaporated to afford crude product. The crude product was purified by flash silica chromatography, elution gradient 0 to 40% ethyl acetate in DCM, to give the desired material as a yellow foam (2.85 g).

NMR Spectrum: ¹H NMR (400.132 MHz, CDCl₃) δ 1.32 (3H, d), 1.88-1.75 (2H, m), 2.16-2.06 (2H, m), 2.34 (3H, s), 2.36 (3H, s), 3.33-3.24 (1H, m), 3.63-3.55 (1H, M), 3.88-3.71 (4H, m), 4.03 (1H, d), 4.18 (1H, d), 4.51-4.43 (1H, m), 6.64 (2H, d), 7.04 (1H, s), 8.01 (2H, d)

LCMS Spectrum: m/z (ESI+)(M+H)+=486; HPLC tR=1.65 min.

### 2-Chloro-4-l [-[(4,5-dimethyl-1,3-thiazol-2-yl)sulfonyl]cyclopropyl]-6-[(3S)-3-methylmorpholin-4-yl]pyrimidine

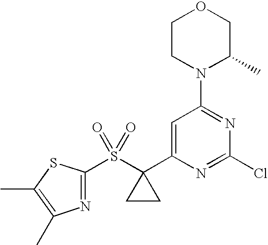

Sodium hydroxide (50% w/w) (45.9 g, 573.32 mmol) was added in one portion to 2-chloro-4-[(4,5-dimethyl-1,3-thiazol-2-yl)sulfonylmethyl]-6-[(3S)-3-methylmorpholin-4-yl]pyrimidine (4.2 g, 10.42 mmol), tetrabutylammonium bromide (0.336 g, 1.04 mmol) and 1,2-dibromoethane (2.69 mL, 31.27 mmol) in toluene (52.1 mL) and the resulting mixture stirred at 60° C. for 5 hours. The reaction mixture was diluted with toluene (50 mL) and water (100 mL) and washed sequentially with water (2×100 mL) and saturated brine (100 mL). The 1o organic layer was dried over MgSO₄, filtered and evaporated to afford crude product. The crude product was purified by flash silica chromatography, elution gradient 0 to 50% ethyl acetate in DCM, to give the desired material as a white foam (3.42 g).

NMR Spectrum: ¹H NMR (400.132 MHz, CDCl₃) δ 1.34 (3H, d), 1.83-1.72 (2H, m), 2.11-2.02 (2H, m), 2.37 (3H, s), 2.44 (3H, s), 3.29 (1H, t), 3.54 (1H, t), 3.72-3.66 (1H, m), 3.82-3.76 (1H, m), 4.13-3.97 (2H, m), 4.35 (1H, s), 7.29 (1H, s)

LCMS Spectrum: m/z (ESI+)(M+H)+=429; HPLC tR=2.35 min.

### 2-Chloro-4-[(4,5-dimethyl-1,3-thiazol-2-yl)sulfonylmethyl]-6-[(3S)-3-methylmorpholin-4-yl]pyrimidine

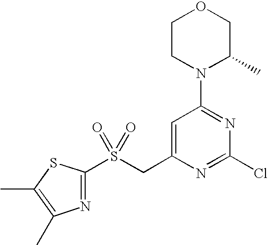

3-Chloroperoxybenzoic acid (11.18 g, 49.88 mmol) was added portionwise to (2-chloro-4-[(4,5-dimethyl-1,3-thiazol-2-yl)sulfanylmethyl]-6-[(3S)-3-methylmorpholin-4-yl]pyrimidine (7.4 g, 19.95 mmol) in DCM (100 mL) at 5° C. over a period of 15 minutes under nitrogen. The resulting suspension was stirred at 5° C. for 1 hour then allowed to warm to RT and stirred for 3 hours. A saturated solution of sodium hydrogen carbonate (100 mL) was added and the organics separated and washed with water (100 mL) and brine (100 mL), dried over MgSO₄ and evaporated in vacuo. The crude product was purified by flash silica chromatography, elution gradient 30 to 60% ethyl acetate in DCM, to give the desired material as a white solid (4.2 g).

NMR Spectrum: ¹H NMR (400.132 MHz, CDCl₃) δ 1.32 (3H, d), 2.42 (3H, s), 2.46 (3H, s), 3.28 (1H, t), 3.54 (1H, t), 3.71-3.66 (1H, m), 3.81-3.76 (1H, m), 4.09-3.96 (2H, m), 4.35-4.23 (1H, m), 4.51 (2H, s), 6.59 (1H, s)

LCMS Spectrum: m/z (ESI+)(M+H)+=403; HPLC tR=2.02 min.

### 2-Chloro-4-[(4,5-dimethyl-1,3-thiazol-2-yl)sulfanylmethyl]-6-[(3S)-3-methylmorpholin-4-yl]pyrimidine

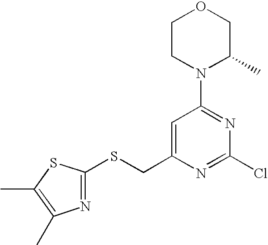

2-Chloro-4-(iodomethyl)-6-[(3S)-3-methylmorpholin-4-yl]pyrimidine (7 g, 19.80 mmol) was added to 4,5-dimethylthiazole-2-thiol (3.16 g, 21.78 mmol) and DIPEA (5.17 mL, 29.70 mmol) in THF (10 mL) and the slurry stirred at RT for 7 hours. The solvent was removed under reduced pressure and the residue diluted with DCM and washed sequentially with water and saturated brine. The organic layer was dried over MgSO₄, filtered and evaporated to afford crude product. The crude product was purified by flash silica chromatography, elution gradient 0 to 20% ethyl acetate in DCM, to give the desired material as a yellow gum (7.95 g).

NMR Spectrum: ¹H NMR (400 MHz, DMSO-d₆) δ 1.16-1.19 (3H, m), 2.23 (3H, d), 2.29 (3H, d), 3.16-3.20 (1H, td), 3.39-3.46 (1H, td), 3.55-3.59 (1H, dd), 3.71 (1H, d), 3.91-3.94 (2H, dd), 4.21-4.29 (3H, m), 6.80 (1H, s)

LCMS Spectrum: m/z (ESI+)(M+H)+=371; HPLC tR=2.31 min.

The preparation of 2-chloro-4-(iodomethyl)-6-[(3S)-3-methylmorpholin-4-yl]pyrimidine was described earlier.

### EXAMPLE 81

### 1-[4-[4-[1-[(4,5-Dimethyl-1,3-thiazol-2-yl)sulfonyl]cyclopropyl]-6-[(3S)-3-methylmorpholin-4-yl]pyrimidin-2-yl]phenyl]-3-(1-methylpyrazol-4-yl)urea

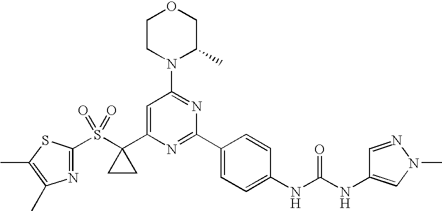

Phenyl N-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]carbamate (150 mg, 0.25 mmol), 1-methyl-1H-pyrazol-4-amine dihydrochloride (126 mg, 0.74 mmol) and DIPEA (0.428 mL, 2.48 mmol) were dissolved in dioxane (10 mL) and sealed into a microwave tube. The reaction was heated to 100° C. for 150 minutes in the microwave reactor and cooled to RT. The solvent was evaporated under reduced pressure, and the crude product was purified by preparative HPLC, eluting with decreasingly polar mixtures of water (containing 1% ammonia) and acetonitrile, to give the desired material as a white solid (70 mg).

NMR Spectrum: ¹H NMR (400.132 MHz, DMSO-d₆) δ 1.22 (3H, d), 1.77-1.72 (2H, m), 1.95-1.89 (2H, m), 2.37 (3H, s), 2.41 (3H, s), 3.24-3.14 (1H, m), 3.52-3.43 (1H, m), 3.65-3.59 (1H, m), 3.81-3.75 (4H, m), 4.00-3.95 (1H, m), 4.22-4.11 (1H, m), 4.51-4.41 (1H, m), 6.79 (1H, s), 7.38 (1H, s), 7.47 (2H, d), 7.77 (1H, s), 7.92 (2H, d), 8.36 (1H, s), 8.83 (1H, s)

LCMS Spectrum: m/z (ESI+)(M+H)+=609; HPLC tR=2.09 min.

The preparation of phenyl N-[4-[4-[1-[(4,5-dimethyl-1,3-thiazol-2-yl)sulfonyl]cyclopropyl]-6-[(3S)-3-methylmorpholin-4-yl]pyrimidin-2-yl]phenyl]carbamate was described earlier.

### EXAMPLE 82

1-[4-[4-[1-(4-Fluoro-2-methylphenyl)sulfonylcyclopropyl]-6-[(3S)-3-methylmorpholin-4-yl]pyrimidin-2-yl]phenyl]-3-methylurea

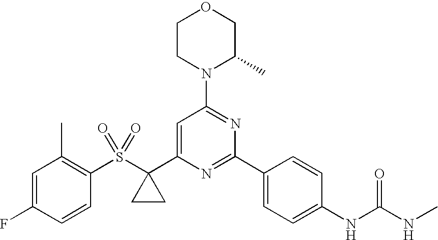

Phenyl N-[4-[4-[1-(4-fluoro-2-methylphenyl)sulfonylcyclopropyl]-6-[(3S)-3-methylmorpholin-4-yl]pyrimidin-2-yl]phenyl]carbamate (0.150 g, 0.25 mmol), triethylamine (0.104 mL, 0.75 mmol) and methylamine (0.75 mmol) were dissolved in dioxane (10 mL) and heated at 50° C. overnight. The reaction was evaporated to dryness and was purified by preparative HPLC, eluting with using decreasingly polar mixtures of water (containing 1% ammonia) and acetonitrile, to give the desired material as a white solid.

NMR Spectrum: ¹H NMR (400.132 MHz, DMSO-d₆) δ 1.16 (3H, d), 1.63-1.60 (2H, m), 1.85-1.83 (2H, m), 2.44 (3H, s), 2.66 (3H, d), 3.13 (1H, ddd), 3.45 (1H, ddd), 3.60 (1H, dd), 3.74 (1H, d), 3.95 (1H, dd), 4.10 (1H, d), 4.42 (1H, s), 6.04 (1H, q), 6.62 (1H, s), 7.17 (1H, ddd), 7.24 (1H, dd), 7.40 (2H, d), 7.84-7.81 (3H, m), 8.71 (1H, s);

LCMS Spectrum: m/z (ES+) (M+H)+=540; HPLC tR=2.34 min

The following compounds were prepared in an analogous fashion from phenyl N-[4-[4-[1-(4-fluoro-2-methylphenyl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 82a | 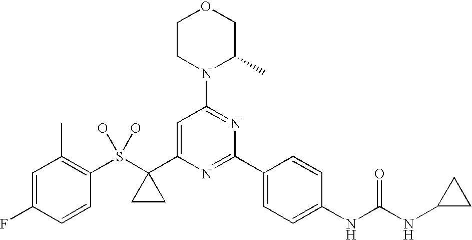 | 3-cyclopropyl-1-[4-[4-[1-(4-fluoro-2-methylphenyl)sulfonylcyclopropyl]-6-[(3S)-3-methylmorpholin-4-yl]pyrimidin-2-yl]phenyl]urea | 566 | 2.51 |
| 82b | 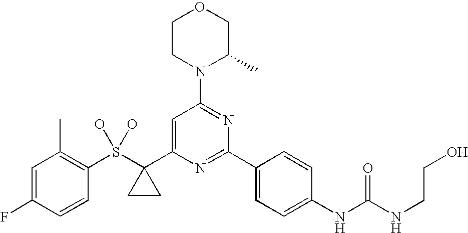 | 1-[4-[4-[1-(4-fluoro-2-methylphenyl)sulfonylcyclopropyl]-6-[(3S)-3-methylmorpholin-4-yl]pyrimidin-2-yl]phenyl]-3-(2-hydroxyethyl)urea | 570 | 2.15 |
| 82c | 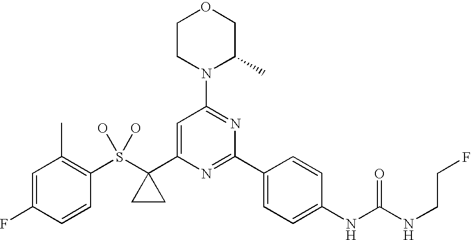 | 3-(2-fluoroethyl)-1-[4-[4-[1-(4-fluoro-2-methylphenyl)sulfonylcyclopropyl]-6-[(3S)-3-methylmorpholin-4-yl]pyrimidin-2-yl]phenyl]urea | 572 | 2.50 |
| 82d | 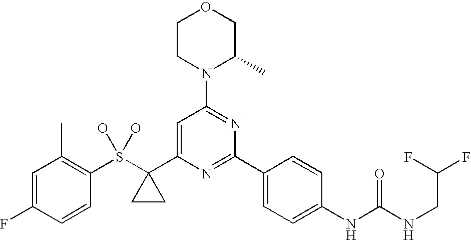 | 3-(2,2-difluoroethyl)-1-[4-[4-[1-(4-fluoro-2-methylphenyl)sulfonylcyclopropyl]-6-[(3S)-3-methylmorpholin-4-yl]pyrimidin-2-yl]phenyl]urea | 590 | 2.62 |
| 82e | 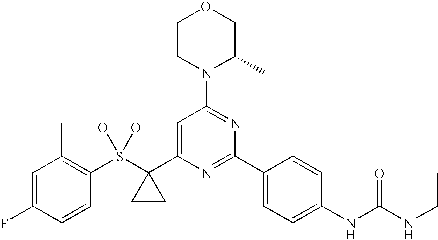 | 3-ethyl-1-[4-[4-[1-(4-fluoro-2-methylphenyl)sulfonylcyclopropyl]-6-[(3S)-3-methylmorpholin-4-yl]pyrimidin-2-yl]phenyl]urea | 554 | 2.50 |
| 82f | 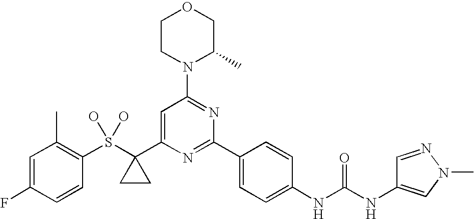 | 1-[4-[4-[1-(4-fluoro-2-methylphenyl)sulfonylcyclopropyl]-6-[(3S)-3-methylmorpholin-4-yl]pyrimidin-2-yl]phenyl]-3-(1-methylpyrazol-4-yl)urea | 606 | 2.36 |

### EXAMPLE 82a

¹H NMR (400.132 MHz, DMSO-d₆) δ 1.16 (3H, d), 1.63-1.60 (2H, m), 1.85-1.83 (2H, m), 2.44 (3H, s), 2.66 (3H, d), 3.13 (1H, ddd), 3.45 (1H, ddd), 3.60 (1H, dd), 3.74 (1H, d), 3.95 (1H, dd), 4.10 (1H, d), 4.42 (1H, s), 6.04 (1H, q), 6.62 (1H, s), 7.17 (1H, ddd), 7.24 (1H, dd), 7.40 (2H, d), 7.84-7.81 (3H, m), 8.71 (1H, s);

### EXAMPLE 82b

¹H NMR (400.132 MHz, DMSO-d₆) δ 1.16 (3H, d), 1.63-1.60 (2H, m), 1.85-1.83 (2H, m), 2.44 (3H, s), 3.20-3.09 (3H, m), 3.48-3.42 (3H, m), 3.60 (1H, dd), 3.74 (1H, d), 3.95 (1H, dd), 4.10 (1H, d), 4.42 (1H, s), 4.73 (1H, t), 6.23 (1H, t), 6.62 (1H, s), 7.17 (1H, ddd), 7.24 (1H, dd), 7.39 (2H, d), 7.85-7.80 (3H, m), 8.77 (1H, s);

### EXAMPLE 82c

¹H NMR (400.132 MHz, DMSO-d₆) δ 1.16 (3H, d), 1.63-1.60 (2H, m), 1.85-1.83 (2H, m), 2.44 (3H, s), 3.13 (1H, ddd), 3.47-3.36 (3H, m), 3.60 (1H, dd), 3.74 (1H, d), 3.95 (1H, dd), 4.10 (1H, d), 4.55-4.40 (3H, m), 6.41 (1H, t), 6.63 (1H, s), 7.17 (1H, ddd), 7.24 (1H, dd), 7.40 (2H, d), 7.86-7.81 (3H, m), 8.78 (1H, s);

### EXAMPLE 82d

¹H NMR (400.132 MHz, DMSO-d₆) δ 1.16 (3H, d), 1.63-1.60 (2H, m), 1.86-1.83 (2H, m), 2.44 (3H, s), 3.13 (1H, ddd), 3.62-3.42 (4H, m), 3.74 (1H, d), 3.95 (1H, dd), 4.10 (1H, d), 4.42 (1H, s), 6.07 (1H, ddt), 6.50 (1H, t), 6.63 (1H, s), 7.17 (1H, ddd), 7.24 (1H, dd), 7.41 (2H, d), 7.86-7.80 (3H, m), 8.90 (1H, s);

### EXAMPLE 82e

¹H NMR (400.132 MHz, DMSO-d₆) δ 1.07 (3H, t), 1.16 (3H, d), 1.63-1.60 (2H, m), 1.85-1.83 (2H, m), 2.44 (3H, s), 3.18-3.09 (3H, m), 3.44 (1H, ddd), 3.60 (1H, dd), 3.74 (1H, d), 3.95 (1H, ddd), 4.42 (1H, s), 6.13 (1H, t), 6.62 (1H, s), 7.17 (1H, ddd), 7.24 (1H, dd), 7.39 (2H, d), 7.84-7.81 (3H, m), 8.63 (1H, s);

### EXAMPLE 82f

¹H NMR (400.132 MHz, DMSO-d₆) δ 1.17 (3H, d), 1.64-1.61 (2H, m), 1.86-1.83 (2H, m), 2.45 (3H, s), 3.14 (1H, ddd), 3.45 (1H, ddd), 3.60 (1H, dd), 3.74 (1H, d), 3.79 (3H, s), 3.96 (1H, dd), 4.11 (1H, d), 4.43 (1H, s), 6.64 (1H, s), 7.18 (1H, ddd), 7.25 (1H, dd), 7.38 (1H, s), 7.45 (2H, d), 7.77 (1H, s), 7.88-7.81 (3H, m), 8.36 (1H, s), 8.81 (1H, s);

The preparation of phenyl N-[4-[4-[1-(4-fluoro-2-methylphenyl)sulfonylcyclopropyl]-6-[(3S)-3-methylmorpholin-4-yl]pyrimidin-2-yl]phenyl]carbamate is described below.

### Phenyl N-[4-[4-[1-(4-fluoro-2-methylphenyl)sulfonylcyclopropyl]-6-[(3S)-3-methylmorpholin-4-yl]pyrimidin-2-yl]phenyl]carbamate

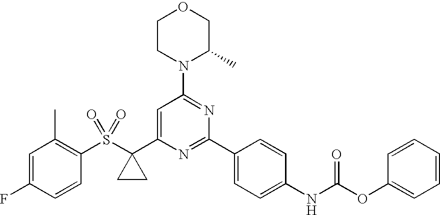

4-[4-[1-(4-Fluoro-2-methylphenyl)sulfonylcyclopropyl]-6-[(3S)-3-methylmorpholin-4-yl]pyrimidin-2-yl]aniline (as the hydrochloride salt) (1.80 g, 3.47 mmol) and sodium bicarbonate (2.91 g, 34.68 mmol) were added to DCM (60 mL) and stirred for 10 minutes. Phenyl chloroformate (0.566 mL, 4.51 mmol) was added slowly and the reaction stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. The crude product was purified by flash silica chromatography, elution gradient 30 to 60% ethyl acetate in isohexane, to give the desired material as a yellow solid (1.73 g).

NMR Spectrum: ¹H NMR (400.132 MHz, DMSO-d₆) δ 1.17 (3H, d), 1.65-1.62 (2H, m), 1.87-1.84 (2H, m), 2.45 (3H, s), 3.14 (1H, ddd), 3.49-3.42 (1H, m), 3.60 (1H, dd), 3.74 (1H, d), 3.95 (1H, dd), 4.12 (1H, d), 4.45 (1H, s), 6.67 (1H, s), 7.17 (1H, ddd), 7.30-7.23 (4H, m), 7.45 (2H, t), 7.54 (2H, d), 7.83 (1H, dd), 7.93 (2H, d), 10.40 (1H, s);

LCMS Spectrum: m/z (ES+) (M+H)+=603; HPLC tR=3.15 min

### 5 4-[4-l [-(4-Fluoro-2-methylphenyl)sulfonylcyclopropyl]-6-[(3S)-3-methylmorpholin-4-yl]pyrimidin-2-yl]aniline

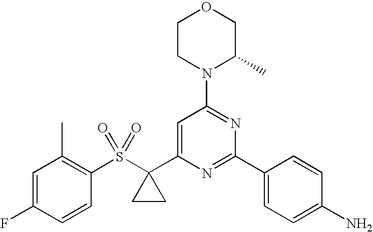

tert-Butyl N-[4-[4-[1-(4-fluoro-2-methylphenyl)sulfonylcyclopropyl]-6-[(3S)-3-methylmorpholin-4-yl]pyrimidin-2-yl]phenyl]carbamate (2.03 g, 3.48 mmol) was added to 6.0 N hydrogen chloride in propan-2-ol (30 mL) and stirred for 2 hours at RT. The crude solution was triturated with diethyl ether to give the desired material (as the hydrochloride salt) as a white solid (1.80 g).

NMR Spectrum: ¹H NMR (400.132 MHz, DMSO-d₆) δ 1.04 (3H, d), 1.70-1.65 (2H, m), 1.89-1.86 (2H, m), 2.45 (3H, s), 3.22 (1H, ddd), 3.44 (1H, ddd), 3.59 (1H, dd), 3.75 (1H, d), 3.97 (1H, dd), 4.21 (1H, s), 4.50 (1H, s), 6.14 (2H, s), 6.70 (1H, s), 7.14-7.03 (2H, m), 7.21 (1H, t), 7.31 (1H, d), 7.82 (1H, dd), 7.93 (2H, d);

LCMS Spectrum: m/z (ES+) (M+H)+=483; HPLC tR=2.67 min

### tert-Butyl N-[4-[4-[1-(4-fluoro-2-methylphenyl)sulfonylcyclopropyl]-6-[(3S)-3-methylmorpholin-4-yl]pyrimidin-2-yl]phenyl]carbamate

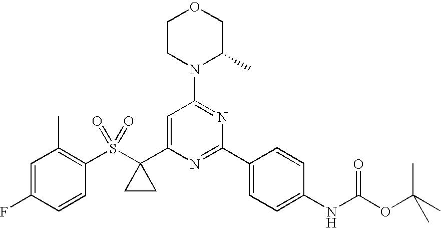

To tert-butyl N-[4-[4-[(4-fluoro-2-methylphenyl)sulfonylmethyl]-6-[(3S)-3-methylmorpholin-4-yl]pyrimidin-2-yl]phenyl]carbamate (3.36 g, 6.04 mmol) in DMF (30 mL) was added rapidly sodium hydride (1.159 g, 24.14 mmol), this was stirred at RT for 10 minutes before the slow addition 1,2-dibromoethane (2.081 mL, 24.14 mmol) in DMF (30 mL). The resulting suspension was stirred at RT for 1 hour. A further portion of sodium hydride (0.58 g, 12.07 mmol) and 1,2 dibromoethane (1.04 mL, 12.07 mmol) were rapidly added and the reaction was stirred for a further 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to give the desired material as a white foam (2.05 g).

NMR Spectrum: ¹H NMR (400.132 MHz, DMSO-d₆) δ 1.16 (3H, d), 1.49 (9H, s), 1.63-1.60 (2H, m), 1.86-1.83 (2H, m), 2.44 (3H, s), 3.13 (1H, ddd), 3.45 (1H, ddd), 3.60 (1H, dd), 3.74 (1H, d), 3.95 (1H, dd), 4.11 (1H, d), 4.43 (1H, s), 6.65 (1H, s), 7.17 (1H, ddd), 7.24 (1H, dd), 7.47 (2H, d), 7.82 (1H, dd), 7.86 (2H, d), 9.50 (1H, s);

LCMS Spectrum: m/z (ES+) (M+H)+=583; HPLC tR=3.17 min

### tert-Butyl N-[4-[4-[(4-fluoro-2-methylphenyl)sulfonylmethyl]-6-[(3S)-3-methylmorpholin-4-yl]pyrimidin-2-yl]phenyl]carbamate

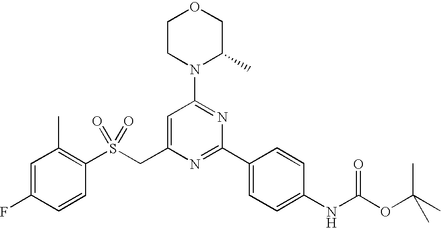

Sodium 4-fluoro-2-methylbenzenesulfinate (1.663 g, 7.84 mmol) and tert-butyl N-[4-[4-(iodomethyl)-6-[(3S)-3-methylmorpholin-4-yl]pyrimidin-2-yl]phenyl]carbamate (4.0 g, 7.84 mmol) were dissolved in DMF (25 mL) and stirred for 1 hour at RT. The solvent was evaporated to afford a yellow solid. This was partitioned between aqueous sodium thiosulphate solution (50 mL) and DCM (75 mL). The organics were purified by flash silica chromatography, elution gradient 30 to 60% ethyl acetate in isohexane, to give the desired material as a white foam (4.20 g).

NMR Spectrum: ¹H NMR (400.132 MHz, DMSO-d₆) δ 1.20 (3H, d), 1.49 (9H, s), 2.65 (3H, s), 3.16 (1H, ddd), 3.48 (1H, ddd), 3.63 (1H, dd), 3.76 (1H, d), 3.97 (1H, dd), 4.11 (1H, d), 4.39 (1H, s), 4.67 (2H, s), 6.67 (1H, s), 7.15 (1H, ddd), 7.39 (1H, dd), 7.45 (2H, d), 7.68 (1H, dd), 7.80 (2H, d), 9.50 (1H, s);

LCMS Spectrum: m/z (ES+) (M+H)+=557; HPLC tR=3.06 min

### Sodium 4-fluoro-2-methylbenzenesulfinate

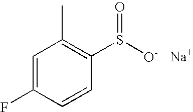

A solution of sodium sulfate (15.10 g, 119.82 mmol) in water (100 mL) was stirred at RT for 10 minutes. Sodium bicarbonate (20.13 g, 239.65 mmol) was added to the stirred solution. The resulting solution was stirred at 50° C. for 10 minutes. 4-Fluoro-2-methylbenzene-1-sulfonyl chloride (25 g, 119.82 mmol) was added portionwise to the solution and was stirred at 50° C. for 2 hours. The reaction mixture was evaporated to dryness and re-dissolved in ethanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (17.5 g).

NMR Spectrum: ¹H NMR (400.132 MHz, DMSO-d₆) δ 2.46 (3H, s), 6.86 (1H, dd), 6.95 (1H, ddd), 7.66 (1H, dd);

The preparation of tert-butyl N-[4-[4-(iodomethyl)-6-[(3S)-3-methylmorpholin-4-yl]pyrimidin-2-yl]phenyl]carbamate was described earlier.

### EXAMPLE 83

### 3-(2-Hydroxyethyl)-1-[4-[4-[(3S)-3-methylmorpholin-4-yl]-6-(1-pyridin-4-ylsulfonylcyclobutyl)pyrimidin-2-yl]phenyl]urea

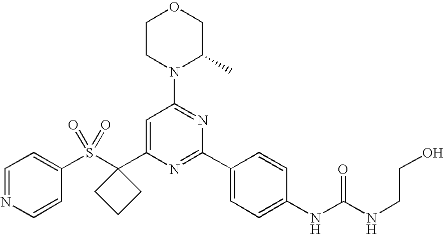

A solution of phenyl N-[4-[4-[(3S)-3-methylmorpholin-4-yl]-6-(1-pyridin-4-ylsulfonylcyclobutyl)pyrimidin-2-yl]phenyl]carbamate (100 mg, 0.17 mmol) in NMP (2 mL) was treated with ethanolamine (11 mg, 0.18 mmol) and triethylamine (51 mg, 0.5 mmol) and stirred at RT overnight. The crude reaction mixture was purified by preparative HPLC, eluting with decreasingly polar mixtures of water (containing 1% ammonia) and acetonitrile, to give the desired material (66mg).

NMR Spectrum: ¹H NMR (400 MHz, DMSO-d₆) δ 1.20-1.22 (3H, m), 1.93 (1H, t), 2.11-2.16 (1H, m), 2.78-2.85 (2H, m), 3.09-3.19 (5H, m), 3.45-3.52 (3H, m), 3.63-3.66 (1H, m), 3.75-3.78 (1H, m), 3.97 (1H, d), 4.15-4.19 (1H, m), 4.51 (1H, s), 4.78 (1H, s), 6.26 (1H, t), 6.63 (1H, s), 7.35 (2H, d), 7.46 (2H, d), 7.68 (2H, d), 8.72 (2H, d), 8.80 (1H, s)

LCMS Spectrum: m/z (ESI+) (M+H)+=553.48; HPLC tR=1.93 min.

The compounds below were prepared in an analogous fashion from phenyl N-[4-[4-[(3S)-3-methylmorpholin-4-yl]-6-(1-pyridin-4-ylsulfonylcyclobutyl)pyrimidin-2-yl]phenyl]carbamate and the appropriate amine.

| | | | LCMS | Retention |
|---|---|---|---|---|
| Example | Structure | NAME | MH+ | time (min) |
| 83a | 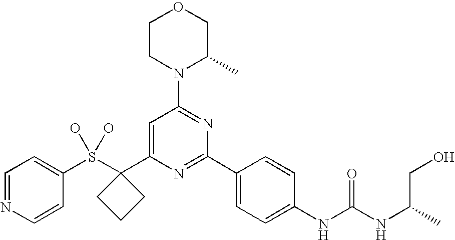 | 3-[(2S)-1-hydroxypropan-2-yl]-1-[4-[4-[(3S)-3-methylmorpholin-4-yl]-6-(1-pyridin-4-ylsulfonylcyclobutyl)pyrimidin-2-yl]phenyl]urea | 567.5 | 2.02 |
| 83b | 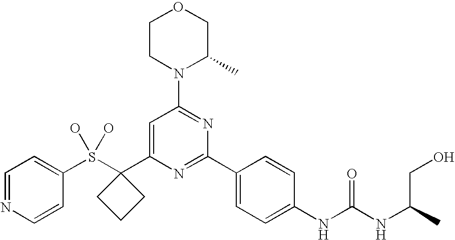 | 3-[(2R)-1-hydroxypropan-2-yl]-1-[4-[4-[(3S)-3-methylmorpholin-4-yl]-6-(1-pyridin-4-ylsulfonylcyclobutyl)pyrimidin-2-yl]phenyl]urea | 567.5 | 2.02 |
| 83c | 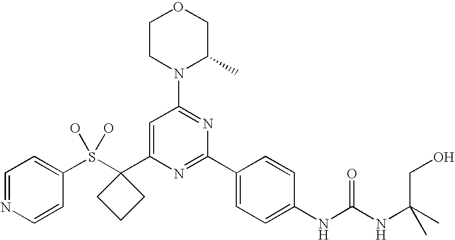 | 3-(1-hydroxy-2-methylpropan-2-yl)-1-[4-[4-[(3S)-3-methylmorpholin-4-yl]-6-(1-pyridin-4-ylsulfonylcyclobutyl)pyrimidin-2-yl]phenyl]urea | 581.6 | 2.22 |
| 83d | 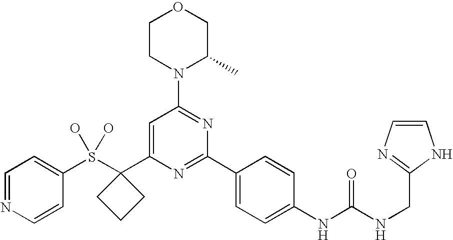 | 3-(1H-imidazol-2-ylmethyl)-1-[4-[4-[(3S)-3-methylmorpholin-4-yl]-6-(1-pyridin-4-ylsulfonylcyclobutyl)pyrimidin-2-yl]phenyl]urea | 589.5 | 1.43 |

### EXAMPLE 83a

¹H NMR (400 MHz, DMSO-d₆) δ 1.07-1.09 (3H, m), 1.20 (3H, d), 1.91-1.93 (1H, m), 2.13 (1H, t), 2.78-2.85 (2H, m), 3.08-3.19 (3H, m), 3.38-3.41 (2H, m), 3.45-3.54 (1H, td), 3.60-3.82 (3H, m), 3.96 (1H, dd), 4.16 (1H, d), 4.50 (1H, s), 4.38 (1H, t), 6.09-6.11 (1H, m), 6.63 (1H, s), 7.34 (2H, d), 7.46-7.47 (2H, m), 7.68 (2H, d), 8.70-8.72 (3H, m)

### EXAMPLE 83b

¹H NMR (400 MHz, DMSO-d₆) δ 1.07-1.09 (3H, m), 1.20 (3H, d), 1.91-1.93 (1H, m), 2.13 (1H, t), 2.78-2.85 (2H, m), 3.08-3.19 (3H, m), 3.38-3.41 (2H, m), 3.45-3.54 (1H, td), 3.60-3.82 (3H, m), 3.96 (1H, dd), 4.16 (1H, d), 4.50 (1H, s), 4.83 (1H, t), 6.09-6.11 (1H, m), 6.63 (1H, s), 7.34 (2H, d), 7.46-7.47 (2H, m), 7.68 (2H,d), 8.70-8.72 (3H, m)

### EXAMPLE 83c

¹H NMR (400 MHz, DMSO-d₆) δ 1.21 (3H, d), 1.24 (6H, s), 1.91-1.93 (1H, m), 2.11-2.16 (1H, m), 2.77-2.84 (2H, m), 3.08-3.20 (3H, m), 3.39 (2H, d), 3.46-3.52 (1H, m), 3.62-3.66 (1H, m), 3.76 (1H, d), 3.95-3.99 (1H, m), 4.17 (1H, d) 4.51 (1H, s), 5.00 (1H, t), 6.00 (1H, s), 6.63 (1H, s), 7.29-7.32 (2H, m), 7.46-7.47 (2H, m), 7.65-7.67 (2H, m), 8.71-8.72 (3H, m)

### EXAMPLE 83d

¹H NMR (400 MHz, DMSO-d₆) δ 1.21 (3H, d), 1.92 (1H, q), 2.11-2.16 (1H, m), 2.81 (2H, s), 3.09-3.18 (3H, m), 3.49 (1H, t), 3.64 (1H, d), 3.76 (1H, d), 3.97 (1H, d), 4.17 (1H, d), 4.32 (2H, d), 4.51 (1H, s), 6.64 (2H, s), 6.84 (1H, s), 7.04 (1H, s), 7.38 (2H, d), 7.46 (2H, d), 7.69 (2H, d), 8.72 (2H, d), 8.92 (1H, s), 11.87 (1H, s)

The preparation of phenyl N-[4-[4-[(3S)-3-methylmorpholin-4-yl]-6-(1-pyridin-4-ylsulfonylcyclobutyl)pyrimidin-2-yl]phenyl]carbamate is described below:

### Phenyl N-[4-[4-[(3S)-3-methylmorpholin-4-yl]-6-(1-pyridin-4-ylsulfonylcyclobutyl)pyrimidin-2-yl]phenyl]carbamate

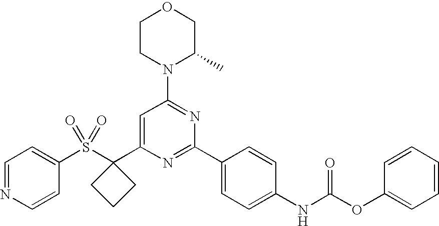

Phenyl chloroformate (0.241 mL, 1.92 mmol) was added dropwise to 4-[4-[(3S)-3-methylmorpholin-4-yl]-6-(1-pyridin-4-ylsulfonylcyclobutyl)pyrimidin-2-yl]aniline (894 mg, 1.92 mmol) and sodium bicarbonate (161 mg, 1.92 mmol) in dioxane (10 mL) at RT under air. The resulting solution was stirred at RT for 2 hours. The reaction was evaporated to dryness and the residue was taken up in water (100 mL) and extracted with ethyl acetate (200 mL). The organic layer was washed with water, brine and then dried (MgSO₄). The solution 1o was evaporated to dryness and the crude material was chromatographed on a silica, eluting with 50% ethyl acetate in isohexane, to give the desired material as a cream coloured solid (690 mg).

NMR Spectrum: ¹H NMR (400 MHz, DMSO-d₆) δ 1.17-1.23 (3H, m), 1.90-1.92 (1H, m), 2.00 (1H, s), 2.12 (1H, t), 2.77-2.83 (2H, m), 3.06-3.17 (3H, m), 3.47-3.50 (1H, m), 3.61-3.65 (1H, m), 3.75 (1H, d), 3.94-3.98 (1H, m), 4.15 (1H, d), 4.50 (1H, s), 5.54 (1H, d), 6.46 (1H, d), 6.53 (1H, s), 6.75-6.79 (2H, m), 7.15-7.19 (1H, m), 7.43-7.49 (2H, m), 7.49 (1H, d), 7.53 (1H, s), 8.70-8.72 (2H, m), 9.34 (1H, s)

LCMS Spectrum: m/z (ESI+) (M+H)+=586.18; HPLC tR=2.87 min.

The preparation of 4-[4-[(3S)-3-methylmorpholin-4-yl]-6-(1-pyridin-4-ylsulfonylcyclobutyl)pyrimidin-2-yl]aniline was described earlier.

### EXAMPLE 84

1-[4-[4-[(3S)-3-Ethylmorpholin-4-yl]-6-(1-methylsulfonylcyclopropyl)pyrimidin-2-yl]phenyl]-3-methylurea

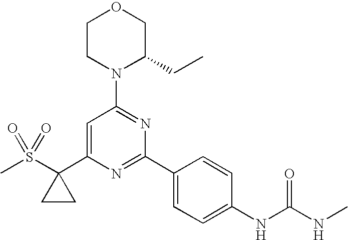

Phenyl N-[4-[4-[(3S)-3-ethylmorpholin-4-yl]-6-(1-methylsulfonylcyclopropyl)pyrimidin-2-yl]phenyl]carbamate (54.0 mg, 0.10 mmol), triethylamine (0.043 mL, 0.31 mmol) and methyl amine (−, 0.31 mmol) were dissolved in dioxane (10 mL) and heated at 50° C. over the weekend. The reaction was evaporated to dryness and was purified by preparative HPLC (Waters XTerra C18 column, 5μ silica, 19 mm diameter, 100 mm length), using decreasingly polar mixtures of water (containing 1% NH3) and MeCN as eluents. Fractions containing the desired compound were evaporated to dryness to afford (S)-1-(4-(4-(3-ethylmorpholino)-6-(1-(methylsulfonyl)cyclopropyl)pyrimidin-2-yl)phenyl)-3-methylurea as a white solid.

NMR Spectrum: ¹H NMR (400.132 MHz, DMSO-d₆) δ 0.89 (3H, t), 1.56-1.54 (2H, m), 1.69-1.64 (2H, m), 1.83-1.69 (2H, m), 2.66 (3H, d), 3.22-3.14 (1H, m), 3.27 (3H, s), 3.47 (1H, ddd), 3.55 (1H, dd), 3.87 (1H, d), 3.93 (1H, dd), 4.41-4.27 (2H, m), 6.07 (1H, q), 6.78 (1H, s), 7.50 (2H, d), 8.19 (2H, d), 8.74 (1H, s);

LCMS Spectrum: m/z (ESI+) (M+H)+=460; HPLC tR=1.98 min.

The following compounds were prepared in an analogous fashion from phenyl N-[4-[4-[(3S)-3-ethylmorpholin-4-yl]-6-(1-methylsulfonylcyclopropyl)pyrimidin-2-yl]phenyl]carbamate and the appropriate amine.

| | | | LCMS | Retention |
|---|---|---|---|---|
| Example | Structure | NAME | MH+ | time (min) |
| 84a | 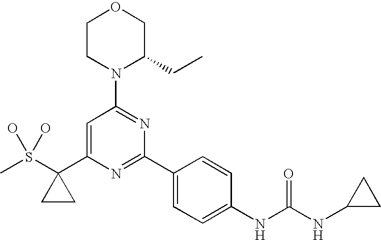 | 3-cyclopropyl-1-[4-[4-[(3S)-3-ethylmorpholin-4-yl]-6-(1-methylsulfonylcyclopropyl)pyrimidin-2-yl]phenyl]urea | 486 | 2.15 |
| 84b | 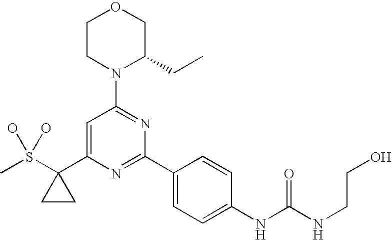 | 1-[4-[4-[(3S)-3-ethylmorpholin-4-yl]-6-(1-methylsulfonylcyclopropyl)pyrimidin-2-yl]phenyl]-3-(2-hydroxyethyl)urea | 490 | 1.8 |
| 84c | 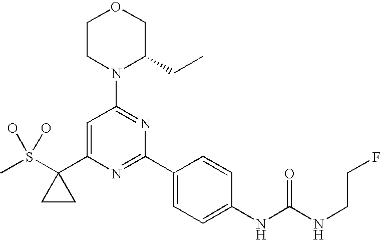 | 1-[4-[4-[(3S)-3-ethylmorpholin-4-yl]-6-(1-methylsulfonylcyclopropyl)pyrimidin-2-yl]phenyl]-3-(2-fluoroethyl)urea | 492 | 2.13 |
| 84d | 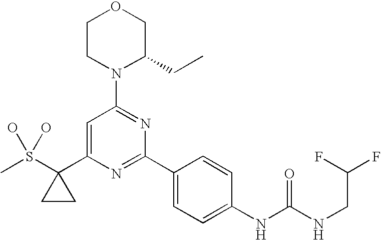 | 3-(2,2-difluoroethyl)-1-[4-[4-[(3S)-3-ethylmorpholin-4-yl]-6-(1-methylsulfonylcyclopropyl)pyrimidin-2-yl]phenyl]urea | 510 | 2.27 |
| 84e | 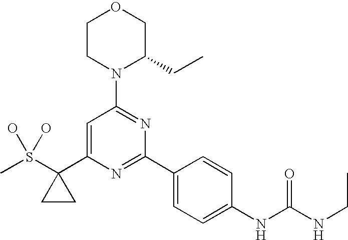 | 3-ethyl-1-[4-[4-[(3S)-3-ethylmorpholin-4-yl]-6-(1-methylsulfonylcyclopropyl)pyrimidin-2-yl]phenyl]urea | 474 | 2.13 |
| 84f | 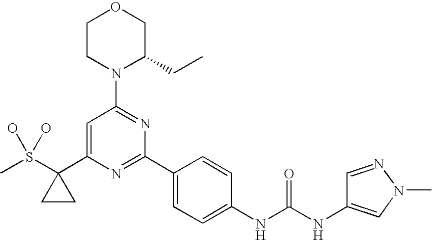 | 1-[4-[4-[(3S)-3-ethylmorpholin-4-yl]-6-(1-methylsulfonylcyclopropyl)pyrimidin-2-yl]phenyl]-3-(1-methylpyrazol-4-yl)urea | 526 | 2.03 |

### EXAMPLE 84a

¹H NMR (400.132 MHz, DMSO-d₆) δ 0.44-0.40 (2H, m), 0.65-0.64 (2H, m), 0.89 (3H, t), 1.57-1.53 (2H, m), 1.66 (2H, m), 1.68-1.64 (2H, m), 3.23-3.14 (1H, m), 3.50-3.44 (1H, m), 3.55 (1H, dd), 3.87 (1H, d), 3.94-3.92 (1H, m), 4.47-4.22 (2H, m), 6.44 (1H, s), 6.78 (1H, s), 7.51 (2H, d), 8.19 (2H, d), 8.54 (1H, s); 4 protons missing due to very weak sample!!

### EXAMPLE 84b

¹H NMR (400.132 MHz, DMSO-d₆) δ 0.89 (3H, t), 1.57-1.53 (2H, m), 1.69-1.64 (2H, m), 1.83-1.71 (2H, m), 3.22-3.16 (3H, m), 3.27 (3H, s), 3.49-3.44 (3H, m), 3.55 (1H, dd), 3.87 (1H, d), 3.94-3.92 (1H, m), 4.39-4.24 (2H, m), 4.73 (1H, t), 6.25 (1H, t), 6.78 (1H, s), 7.49 (2H, d), 8.19 (2H, d), 8.80 (1H, s);

### EXAMPLE 84c

¹H NMR (400.132 MHz, DMSO-d₆) δ 0.89 (3H, t), 1.58-1.54 (2H, m), 1.69-1.64 (2H, m), 1.83-1.69 (2H, m), 3.22-3.17 (1H, m), 3.29 (s, 3H), 3.49-3.37 (3H, m), 3.55 (1H, dd), 3.87 (1H, d), 3.94-3.92 (1H, m), 4.40-4.16 (2H, m), 4.48 (2H, dt), 6.44 (1H, t), 6.78 (1H, s), 7.50 (2H, d), 8.20 (2H, d), 8.81 (1H, s);

### EXAMPLE 84d

¹H NMR (400.132 MHz, DMSO-d₆) δ 0.89 (3H, t), 1.57-1.54 (2H, m), 1.67-1.64 (2H, m), 1.83-1.69 (2H, m), 3.22-3.16 (1H, m), 3.29 (3H, s), 3.60-3.44 (4H, m), 3.87 (1H, d), 3.93 (1H, dd), 4.48-4.21 (2H, m), 6.07 (1H, tt), 6.53 (1H, t), 6.79 (1H, s), 7.51 (2H, d), 8.21 (2H, d), 8.92 (1H, s);

### EXAMPLE 84e

¹H NMR (400.132 MHz, DMSO-d₆) δ 0.89 (3H, t), 1.07 (3H, t), 1.56-1.54 (2H, m), 1.68-1.65 (2H, m), 1.83-1.69 (2H, m), 3.22-3.09 (3H, m), 3.29 (3H, s), 2.47 (1H, ddd), 3.55 (1H, dd), 3.87 (1H, d), 3.93 (1H, dd), 4.40-4.24 (2H, m), 6.16 (1H, t), 6.78 (1H, s), 7.50 (2H, d), 8.19 (2H, d), 8.66 (1H, s);

### EXAMPLE 84f

¹H NMR (400.132 MHz, DMSO-d₆) δ 0.89 (3H, t), 1.57-1.54 (2H, m), 1.69-1.65 (2H, m), 1.84-1.70 (2H, m), 3.23-3.17 (1H, m), 3.47 (1H, ddd), 3.56 (1H, dd), 3.79 (3H, s), 3.88 (1H, d), 3.94 (1H, dd), 4.41-4.23 (2H, m), 6.80 (1H, s), 7.38 (1H, s), 7.55 (2H, d), 7.76 (1H, s), 8.23 (2H, d), 8.39 (1H, s), 8.84 (1H, s); Methyl sulfone hidden under water peak (3H missing).

The preparation of phenyl N-[4-[4-[(3S)-3-ethylmorpholin-4-yl]-6-(1-methylsulfonylcyclopropyl)pyrimidin-2-yl]phenyl]carbamate is described below.

### Phenyl N-[4-[4-[(3S)-3-ethylmorpholin-4-yl]-6-(1-methylsulfonylcyclopropyl)pyrimidin-2-yl]phenyl]carbamate

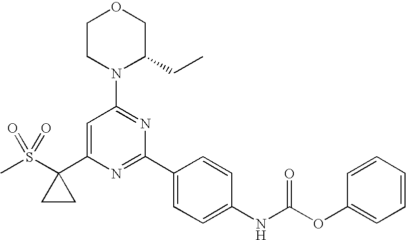

4-[4-[(3S)-3-Ethylmorpholin-4-yl]-6-(1-methylsulfonylcyclopropyl)pyrimidin-2-yl]aniline (as the hydrochloride salt) (0.43 g, 0.98 mmol) and sodium bicarbonate (0.823 g, 9.80 mmol) were added to DCM (60 mL) and stirred for 10 minutes. Phenyl chloroformate (0.160 mL, 1.27 mmol) was added slowly and the reaction stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. The crude product was purified by flash silica chromatography, elution gradient 30 to 60% ethyl acetate in isohexane, to give the desired material as a yellow solid (0.46 g).

NMR Spectrum: ¹H NMR (400.132 MHz, DMSO-d₆) δ 0.89 (3H, t), 1.58-1.55 (2H, m), 1.69-1.65 (2H, m), 1.84-1.70 (2H, m), 3.20 (1H, ddd), 3.27 (3H, s), 3.47 (1H, ddd), 3.56 (1H, dd), 3.87 (1H, d), 3.93 (1H, dd), 4.41-4.24 (2H, m), 6.82 (1H, s), 7.30-7.24 (3H, m), 7.45 (2H, t), 7.64 (2H, d), 8.29 (2H, d), 10.44 (1H, s);

LCMS Spectrum: m/z (ESI+) (M+H)+=523; HPLC tR=2.85 min.

### 4-[4-[(3S)-3-Ethylmorpholin-4-yl]-6-(1-methylsulfonylcyclopropyl)pyrimidin-2-yl]aniline

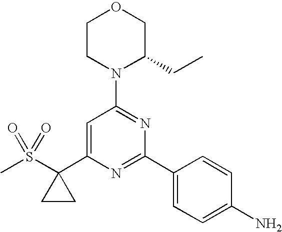

tert-Butyl N-[4-[4-[(3S)-3-ethylmorpholin-4-yl]-6-(1-methylsulfonylcyclopropyl)pyrimidin-2-yl]phenyl]carbamate (0.5 g, 0.99 mmol) was added to 6.0 N hydrogen chloride in propan-2-ol (30 mL) and stirred for 2 hours at RT. The crude solution was triturated with diethyl ether to give the desired material (as the hydrochloride salt) as a yellow solid (0.43 g).

NMR Spectrum: ¹H NMR (400.132 MHz, DMSO-d₆) δ 0.89 (3H, t), 1.65-1.60 (2H, m), 1.75-1.67 (2H, m), 1.83-1.80 (2H, m), 3.24 (3H, s), 3.38-3.28 (1H, m), 3.52-3.46 (1H, m), 3.58 (1H, dd), 3.89 (1H, d), 3.96 (1H, dd), 4.48-4.33 (2H, m), 7.09-6.98 (3H, m), 8.21-8.08 (2H, m); NH2 missing;

LCMS Spectrum: m/z (ESI+) (M+H)+=403; HPLC tR=2.23 min.

### tert-Butyl N-[4-[4-[(3S)-3-ethylmorpholin-4-yl]-6-(1-methylsulfonylcyclopropyl)pyrimidin-2-yl]phenyl]carbamate

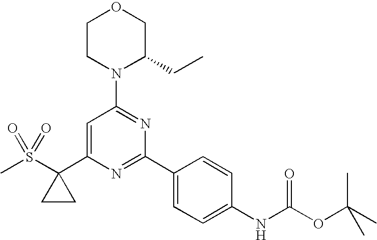

Sodium hydride (0.705 g, 14.69 mmol) was added rapidly to tert-butyl N-[4-[4-[(3S)-3-ethylmorpholin-4-yl]-6-(methylsulfonylmethyl)pyrimidin-2-yl]phenyl]carbamate (1.75 g, 3.67 mmol) in DMF (30 mL) and the mixture stirred at RT for 10 minutes before the slow addition 1,2-dibromoethane (1.266 mL, 14.69 mmol) in DMF (30 mL). The resulting suspension was stirred at RT for 1 hour. The reaction was heated to 40° C. and a further portion of sodium hydride (2.0 eq) and 1,2 dibromoethane (2.0 eq) were rapidly added and the reaction was stirred for a further 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to give the desired material as a yellow foam (0.30 g).

NMR Spectrum: ¹H NMR (400.132 MHz, DMSO-d₆) δ 0.89 (3H, t), 1.50 (9H, s), 1.57-1.54 (2H, m), 1.68-1.66 (2H, m), 1.83-1.70 (2H, m), 3.22-3.16 (1H, m), 3.50-3.44 (1H, m), 3.57-3.53 (1H, m), 3.87 (1H, d), 3.94-3.92 (1H, m), 4.38-4.24 (2H, m), 6.80 (1H, s), 7.57 (2H, d), 8.21 (2H, d), 9.55 (1H, s); methyl peak under water (3H missing).

### tert-Butyl N-[4-[4-[(3S)-3-ethylmorpholin-4-yl]-6-(methylsulfonylmethyl)pyrimidin-2-yl]phenyl]carbamate

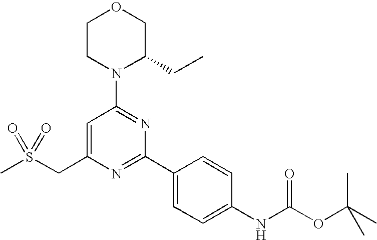

tert-Butyl N-[4-[4-[(3S)-3-ethylmorpholin-4-yl]-6-(iodomethyl)pyrimidin-2-yl]phenyl]carbamate (2.0 g, 3.81 mmol) and sodium methanesulfinate (0.389 g, 3.81 mmol) were dissolved in DMF (25 mL) and stirred for 1 hour at RT. The solvent was evaporated to afford a yellow solid which was partitioned between aqueous sodium thiosulphate solution (50 mL) and DCM (75 mL). The layers were separated, the aquoes layer further extracted with DCM (75 mL), the combined organics purified by flash silica chromatography, elution gradient 30 to 60% ethyl acetate in isohexane, to give the desired material as a white foam (1.7 g).

NMR Spectrum: ¹H NMR (400.132 MHz, DMSO-d₆) δ 0.90 (3H, t), 1.50 (9H, s), 1.73-1.64 (1H, m), 1.85-1.78 (1H, m), 3.24-3.18 (4H, m), 3.48 (1H, ddd), 3.57 (1H, dd), 3.89 (1H, d), 3.95 (1H, dd), 4.30 (2H, s), 4.48 (2H, s), 6.80 (1H, s), 7.57 (2H, d), 8.23 (2H,d), 9.55 (1H, s),

LCMS Spectrum: m/z (ESI+) (M+H)+=477; HPLC tR=2.67 min.

The preparation of tert-butyl N-[4-[4-[(3S)-3-ethylmorpholin-4-yl]-6-(iodomethyl)pyrimidin-2-yl]phenyl]carbamate was described earlier.

### EXAMPLE 85

3-Chloro-4-[1-[2-[4-(methylcarbamoylamino)phenyl]-6-[(3S)-3-methylmorpholin-4-yl]pyrimidin-4-yl]cyclopropyl]sulfonylbenzamide

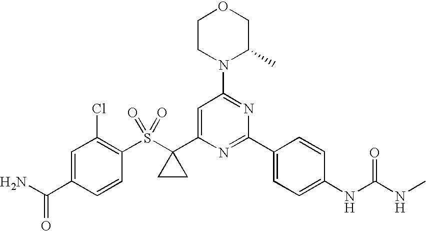

Methylamine (0.347 mL, 0.69 mmol) was added to phenyl N-[4-[4-[1-(4-carbamoyl-2-chlorophenyl)sulfonylcyclopropyl]-6-[(3S)-3-methylmorpholin-4-yl]pyrimidin-2-yl]phenyl]carbamate (150 mg, 0.23 mmol) and triethylamine (0.096 mL, 0.69 mmol) in DMF (0.714 mL) and resulting solution was stirred at 50° C. for 2 hours. The crude mixture was purified by prep HPLC to give the desired material.

NMR Spectrum: ¹H NMR (400.132 MHz, DMSO-d₆) δ 1.15 (3H, d), 1.72-1.76 (2H, m), 1.99-2.02 (2H, m), 2.65 (3H, d), 3.09-3.17 (1H, m), 3.44 (1H, dt), 3.59 (1H, dd), 3.73 (1H, d), 3.94 (1H, d), 4.09 (1H, d), 4.41 (1H, s), 6.03-6.07 (1H, m), 6.61 (1H, 7.35 (2H, d), 7.67 (1H, s), 7.74 (2H, d), 7.88 (1H, dd), 7.99 (1H, d), 8.12 (1H, d), 8.20 (1H, s), 8.68 (1H, s)

LCMS Spectrum: m/z (ES+) (M+H)+=586; HPLC tR=1.86 min.

The compounds below were prepared in an analogous fashion from phenyl N-[4-[4-[1-(4-carbamoyl-2-chlorophenyl)sulfonylcyclopropyl]-6-[(3S)-3-methylmorpholin-4-yl]pyrimidin-2-yl]phenyl]carbamate or phenyl N-[4-[4-[1-(2-chloro-4-cyanophenyl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 85a | 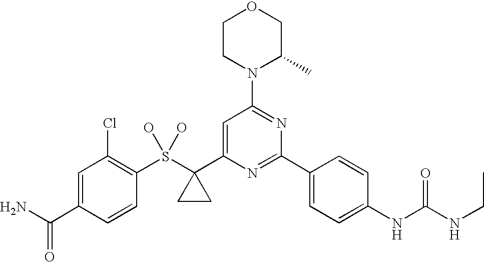 | 3-chloro-4-[1-[2-[4-(ethylcarbamoylamino)phenyl]-6-[(3S)-3-methylmorpholin-4-yl]pyrimidin-4-yl]cyclopropyl]sulfonylbenzamide | 598(M − H)⁻ | 1.98 |
| 85b | 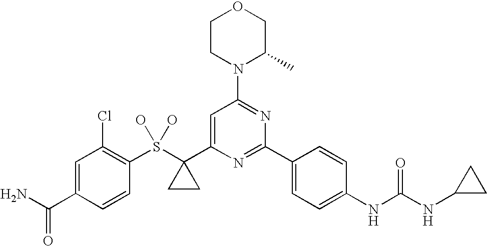 | 3-chloro-4-[1-[2-[4-(cyclopropylcarbamoylamino)phenyl]-6-[(3S)-3-methylmorpholin-4-yl]pyrimidin-4-yl]cyclopropyl]sulfonylbenzamide | 610(M − H)⁻ | 2.00 |
| 85c | 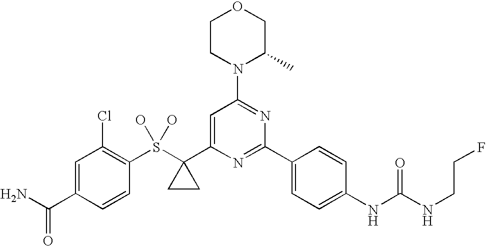 | 3-chloro-4-[1-[2-[4-(2-fluoroethylcarbamoylamino)phenyl]-6-[(3S)-3-methylmorpholin-4-yl]pyrimidin-4-yl]cyclopropyl]sulfonylbenzamide | 616(M − H)⁻ | 1.98 |
| 85d | 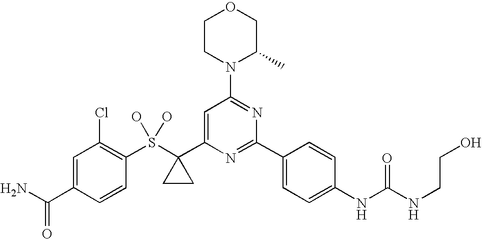 | 3-chloro-4-[1-[2-[4-(2-hydroxyethylcarbamoylamino)phenyl]-6-[(3S)-3-methylmorpholin-4-yl]pyrimidin-4-yl]cyclopropyl]sulfonylbenzamide | 615 | 1.76 |
| 85e | 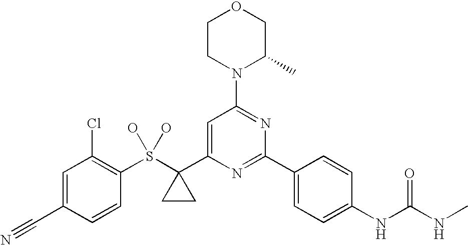 | 1-[4-[4-[1-(2-chloro-4-cyanophenyl)sulfonylcyclopropyl]-6-[(3S)-3-methylmorpholin-4-yl]pyrimidin-2-yl]phenyl]-3-methylurea | 568 | 2.25 |
| 85f | 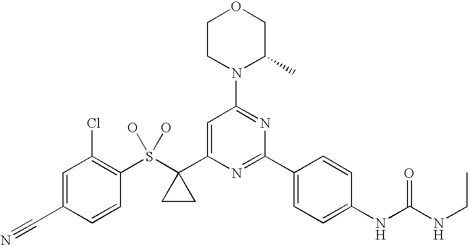 | 3-[4-[4-[1-(2-chloro-4-cyanophenyl)sulfonylcyclopropyl]-6-[(3S)-3-methylmorpholin-4-yl]pyrimidin-2-yl]phenyl]-1-ethylurea | 582 | 2.39 |
| 85g | 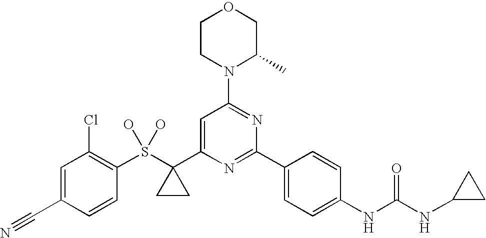 | 1-[4-[4-[1-(2-chloro-4-cyanophenyl)sulfonylcyclopropyl]-6-[(3S)-3-methylmorpholin-4-yl]pyrimidin-2-yl]phenyl]-3-cyclopropylurea | 592(M − H) | 2.40 |
| 85h | 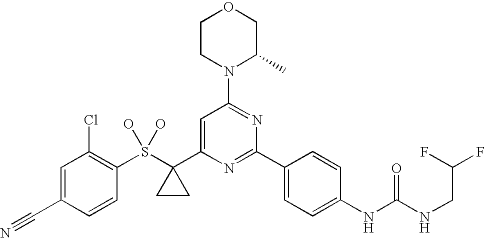 | 1-[4-[4-[1-(2-chloro-4-cyanophenyl)sulfonylcyclopropyl]-6-[(3S)-3-methylmorpholin-4-yl]pyrimidin-2-yl]phenyl]-3-(2,2-difluoroethyl)urea | 618 | 2.51 |
| 85i | 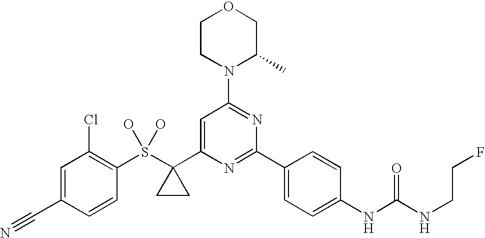 | 1-[4-[4-[1-(2-chloro-4-cyanophenyl)sulfonylcyclopropyl]-6-[(3S)-3-methylmorpholin-4-yl]pyrimidin-2-yl]phenyl]-3-(2-fluoroethyl)urea | 598(M − H)⁻ | 2.39 |
| 85j | 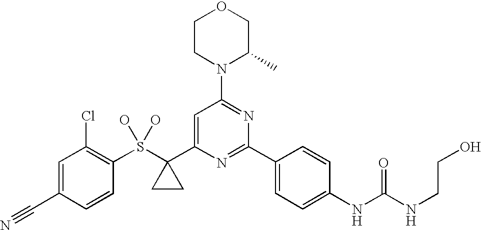 | 1-[4-[4-[1-(2-chloro-4-cyanophenyl)sulfonylcyclopropyl]-6-[(3S)-3-methylmorpholin-4-yl]pyrimidin-2-yl]phenyl]-3-(2-hydroxyethyl)urea | 598 | 2.06 |

### EXAMPLE 85a

¹H NMR (400.132 MHz, DMSO-d₆) δ 1.06 (3H, t), 1.15 (3H, d), 1.72-1.76 (2H, m), 1.99-2.02 (2H, m), 3.09-3.18 (3H, m), 3.44 (1H, dt), 3.59 (1H, dd), 3.73 (1H, d), 3.94 (1H, d), 4.09 (1H, d), 4.41 (1H, s), 6.13 (1H, t), 6.61 (1H, s), 7.34 (2H, d), 7.67 (1H, s), 7.74 (2H, d), 7.88 (1H, dd), 7.99 (1H, d), 8.12 (1H, d), 8.21 (1H, s), 8.60 (1H, s)

### EXAMPLE 85b

¹H NMR (400.132 MHz, DMSO-d₆) δ 0.39-0.43 (2H, m), 0.62-0.67 (2H, m), 1.15 (3H, d), 1.72-1.76 (2H, m), 1.99-2.02 (2H, m), 2.54-2.55 (1H, m), 3.13 (1H, dt), 3.44 (1H, dt), 3.59 (1H, dd), 3.73 (1H, d), 3.94 (1H, dd), 4.09 (1H, d), 4.41 (1H, s), 6.40 (1H, d), 6.62 (1H, s), 7.35 (2H, d), 7.67 (1H, s), 7.74 (2H, d), 7.88 (1H, dd), 7.99 (1H, d), 8.12 (1H, d), 8.21 (1H, s), 8.48 (1H, s)

### EXAMPLE 85c

¹H NMR (400.132 MHz, CDCl₃) δ 1.29 (3H, d), 1.54-1.56 (2H, m), 2.15-2.19 (2H, m), 3.27 (1H, dt), 3.51-3.62 (2H, m), 3.72 (1H, dd), 3.81 (1H, d), 4.02 (1H, dd), 4.08 (1H, d), 4.40 (1H, s), 4.46 (1H, t), 4.58 (1H, t), 5.63 (1H, t), 6.58 (1H, s), 7.17 (1H, s), 7.17 (1H, s), 7.24 (2H, d), 7.45-7.56 (2H, m), 7.64-7.69 (1H, m), 7.84-7.88 (2H, m), 7.90 (2H, d)

### EXAMPLE 85d

¹H NMR (400.132 MHz, DMSO-d₆) δ 1.15 (3H, d), 1.72-1.76 (2H, m), 1.99-2.02 (2H, m), 3.09-3.12 (1H, m), 3.15-3.19 (2H, m), 3.43-3.48 (3H, m), 3.59 (1H, dd), 3.73 (1H, d), 3.93 (1H, dd), 4.09 (1H, d), 4.41 (1H, s), 4.72 (1H, t), 6.24 (1H, t), 6.61 (1H, s), 7.33 (2H, d), 7.66 (1H, s), 7.74 (2H, d), 7.88 (1H, dd), 7.99 (1H, d), 8.12 (1H, d), 8.21 (1H, s), 8.74 (1H, s)

### EXAMPLE 85e

¹H NMR (400.132 MHz, DMSO-d₆) δ 1.18 (3H, d), 1.72-1.78 (2H, m), 2.00-2.03 (2H, m), 2.66 (3H, d), 3.10-3.15 (1H, m), 3.45 (1H, dt), 3.60 (1H, dd), 3.74 (1H, d), 4.06 (1H, q), 4.14 (1H, d), 4.47 (1H, s), 6.04-6.09 (1H, m), 6.66 (1H, s), 7.38 (2H, d), 7.66 (2H, d), 7.90 (1H, d), 8.02 (1H, d), 8.34 (1H, d), 8.71 (1H, s)

### EXAMPLE 85f

¹H NMR (400.132 MHz, DMSO-d₆) δ 1.07 (3H, t), 1.18 (3H, d), 1.73-1.77 (2H, m), 1.99-2.03 (2H, m), 3.09-3.16 (2H, m), 3.16-3.19 (1H, m), 3.45 (1H, dt), 3.60 (1H, dd), 3.74 (1H, d), 3.95 (1H, dd), 4.14 (1H, d), 4.47 (1H, s), 6.16 (1H, t), 6.66 (1H, s), 7.37 (2H, d), 7.66 (2H, d), 7.90 (1H, dd), 8.02 (1H, d), 8.35 (1H, d), 8.62 (1H,s)

### EXAMPLE 85g

¹H NMR (400.132 MHz, DMSO-d₆) δ 0.40-0.44 (2H, m), 0.62-0.67 (2H, m), 1.18 (3H, d), 1.73-1.77 (2H, m), 2.00-2.03 (2H, m), 2.53-2.59 (1H, m), 3.11-3.18 (1H, m), 3.45 (1H, dt), 3.58-3.62 (1H, m), 3.74 (1H, d), 3.95 (1H, dd), 4.14 (1H, d), 4.47 (1H, s), 6.42 (1H, d), 6.66 (1H, s), 7.38 (2H, d), 7.66 (2H, d), 7.90 (1H, dd), 8.02 (1H, d), 8.35 (1H, d), 8.50 (1H, s)

### EXAMPLE 85h

¹H NMR (400.132 MHz, DMSO-d₆) δ 1.18 (3H, d), 1.73-1.77 (2H, m), 2.00-2.03 (2H, m), 3.15 (1H, dt), 3.42-3.51 (1H, m), 3.54-3.62 (3H, m), 3.74 (1H, d), 3.95 (1H, dd), 4.15 (1H, d), 4.47 (1H, s), 6.07 (1H, app t), 6.53 (1H, t), 6.67 (1H, s), 7.39 (2H, d), 7.68 (2H, d), 7.90 (1H, dd), 8.02 (1H, d), 8.35 (1H, d), 8.89 (1H, s)

### EXAMPLE 85i

¹H NMR (400.132 MHz, DMSO-d₆) δ 1.18 (3H, d), 1.73-1.77 (2H, m), 2.00-2.03 (2H, m), 3.15 (1H, dt), 3.39 (1H, q), 3.42-3.48 (2H, m), 3.60 (1H, dd), 3.74 (1H, d), 3.96 (1H, dd), 4.14 (1H, d), 4.42 (1H, t), 4.48 (1H, s), 4.53 (1H, t), 6.44 (1H, t), 6.66 (1H, s), 7.38 (2H, d), 7.67 (2H, d), 7.90 (1H, dd), 8.02 (1H, d), 8.35 (1H, d), 8.77 (1H, s)

### EXAMPLE 85j

¹H NMR (400.132 MHz, DMSO-d₆) δ 1.18 (3H, d), 1.73-1.77 (2H, m), 2.00-2.03 (2H, m), 3.14-3.20 (3H, m), 3.43-3.48 (3H, m), 3.60 (1H, dd), 3.74 (1H, d), 3.95 (1H, dd), 4.14 (1H, d), 4.48 (1H, s), 4.73 (1H, t), 6.26 (1H, t), 6.66 (1H, s), 7.37 (2H, d), 7.66 (2H, d), 7.90 (1H, dd), 8.02 (1H, d), 8.35 (1H, d), 8.77 (1H, s)

The preparation of phenyl N-[4-[4-[1-(4-carbamoyl-2-chlorophenyl)sulfonylcyclopropyl]-6-[(3S)-3-methylmorpholin-4-yl]pyrimidin-2-yl]phenyl]carbamate is described below.

### Phenyl N-[4-[4-l [-(4-carbamoyl-2-chlorophenyl)sulfonylcyclopropyl]-6-[(3S)-3-methylmorpholin-4-yl]pyrimidin-2-yl]phenyl]carbamate

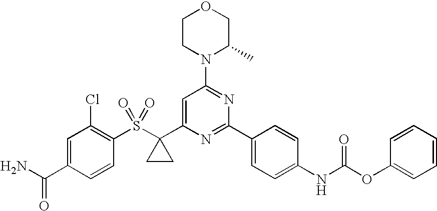

Sodium bicarbonate (0.263 g, 3.12 mmol) was added to 4-[1-[2-(4-aminophenyl)-6-[(3S)-3-methylmorpholin-4-yl]pyrimidin-4-yl]cyclopropyl]sulfonyl-3-chlorobenzamide (1.1 g, 2.08 mmol), in 1,4-dioxane (10.15 mL) and to the resulting suspension was added phenyl chloroformate (0.262 mL, 2.08 mmol) dropwise over 2 minutes. The reaction was stirred at RT for 2 hours then evaporated to dryness and the residue redissolved in DCM (20 mL), and washed with water (20 mL). The organic layer was dried over MgSO₄, filtered and evaporated to afford a solid which was triturated with diethyl ether to give the desired material as a beige solid (0.9 g).

NMR Spectrum: ¹H NMR (400.132 MHz, DMSO-d₆) δ 1.16 (3H, d), 1.73-1.77 (2H, m), 1.99-2.03 (2H, m), 3.12-3.17 (1H, m), 3.40-3.47 (1H, m), 3.57-3.61 (1H, m), 3.73 (1H, d), 3.91-3.96 (1H, m), 4.06-4.10 (1H, m), 4.44 (1H, s), 6.67 (1H, s), 7.24-7.30 (2H, m), 7.45 (2H, t), 7.50 (2H, d), 7.85 (2H, d), 7.88-7.90 (1H, m), 8.00 (1H, d), 8.13 (1H, d), 8.22 (1H, s), 10.40 (1H, s)

LCMS Spectrum: m/z (ES+) (M+H)+=648; HPLC tR=2.62 min.

### 4-[1-[2-(4-Aminophenyl)-6-[(3S)-3-methylmorpholin-4-yl]pyrimidin-4-yl]cyclopropyl]sulfonyl-3-chlorobenzamide

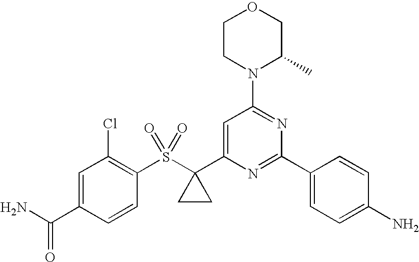

Bis(triphenylphosphine)palladium(II) chloride (0.271 g, 0.39 mmol) was added to 4-(4,4,5,5-tetramethyl-1,3,2-dioxaborolan-2-yl)aniline (2.199 g, 10.04 mmol) and 3-chloro-4-[1-[2-chloro-6-[(3S)-3-methylmorpholin-4-yl]pyrimidin-4-yl]cyclopropyl]sulfonylbenzonitrile (3.5 g, 7.72 mmol) and 2M aqueous solution of sodium carbonate (11.58 mL, 23.16 mmol) in a solvent mixture of DME (20 mL), ethanol (10 mL) and water (10 mL) and the resulting mixture stirred at 95° C. for 16 hours. The reaction mixture was diluted with ethyl acetate (20 mL) and washed with water (2×20 mL). The organic layer was dried over MgSO₄, filtered and evaporated to afford crude product. The crude product was purified by flash silica chromatography, elution gradient 0 to 10% methanol in DCM, to give the desired material as a brown solid (1.1 g).

NMR Spectrum: ¹H NMR (400.132 MHz, DMSO-d₆) δ 1.13 (3H, d), 1.71-1.73 (2H, m), 1.97-2.01 (2H, m), 3.06-3.13 (1H, m), 3.37-3.45 (1H, m), 3.58 (1H, dd), 3.72 (1H, d), 3.92-3.94 (1H, m), 4.04-4.08 (1H, m), 4.37 (1H, s), 5.48 (2H, s), 6.50 (1H, s), 7.58 (2H, d), 7.89 (1H, dd), 8.00 (2H, d), 8.11 (1H, d), 8.21 (1H, s)

LCMS Spectrum: m/z (ES+) (M+H)+=528; HPLC tR=1.99 min.

### 3-Chloro-4-l [-[2-chloro-6-[(3S)-3-methylmorpholin-4-yl]pyrimidin-4-yl]cyclopropyl]sulfonylbenzonitrile

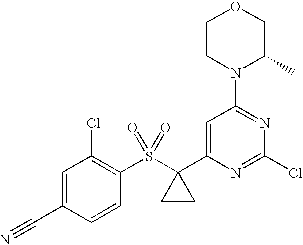

1,2-Dibromoethane (1.654 ml, 19.19 mmol) was added to 3-chloro-4-[[2-chloro-6-[(3S)-3-methylmorpholin-4-yl]pyrimidin-4-yl]methylsulfonyl]benzonitrile (4.1 g, 9.60 mmol), sodium hydroxide (50% w/w) (9.60 mL, 95.95 mmol) and tetrabutylammonium bromide (0.619 g, 1.92 mmol) in DCM and the resulting solution stirred at 40° C. for 5 hours. The 5 reaction mixture was washed sequentially with water (200 mL) and saturated brine (100 mL). The organic layer was dried over MgSO₄, filtered and evaporated to afford crude product. The crude product was purified by flash silica chromatography, elution gradient 5 to 50% ethyl acetate in DCM, to give the desired material as a cream solid (3.50 g).

NMR Spectrum: ¹H NMR (400.132 MHz, DMSO-d₆) δ 1.15 (3H, d), 1.68-1.72 (2H, m), 1.95-1.98 (2H, m), 3.11-3.17 (1H, m), 3.29-3.30 (1H, m), 3.39 (1H, dt), 3.54 (1H, dd), 3.69 (1H, d), 3.91 (1H, dd), 4.30 (1H, s), 6.76 (1H, s), 8.03 (2H, s), 8.33 (1H, s)

LCMS Spectrum: m/z (ESI+) (M+H)+=453; HPLC tR=2.41 min.

### 3-Chloro-4-[[2-chloro-6-[(3S)-3-methylmorpholin-4-yl]pyrimidin-4-yl]methylsulfonyl]benzonitrile

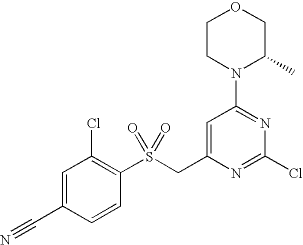

Sodium 2-chloro-4-cyanobenzenesulfinate (4.43 g, 19.80 mmol) was added in one portion to 2-chloro-4-(iodomethyl)-6-[(3S)-3-methylmorpholin-4-yl]pyrimidine (7 g, 19.80 mmol) in acetonitrile (99 mL) and the resulting suspension stirred at 80° C. for 6 hours. The reaction mixture was evaporated to dryness, redissolved in DCM (200 mL) and washed with water (200 mL). The organic layer was dried over MgSO₄, filtered and evaporated to afford crude product. The crude product was purified by flash silica chromatography, elution gradient 0 to 40% ethyl acetate in DCM, to give the desired material as a cream solid (6.20 g).

NMR Spectrum: ¹H NMR (400.132 MHz, DMSO-d₆) δ 1.18 (3H, d), 3.15-3.21 (1H, m), 3.43 (1H, dt), 3.58 (1H, dd), 3.72 (1H, d), 3.93 (2H, m), 4.24 (1H, s), 4.86 (2H, s), 6.87 (1H, s), 7.96 (1H, d), 8.06 (1H, dd), 8.43 (1H, d)

LCMS Spectrum: m/z (ESI+) (M+H)+=427; HPLC tR=2.28 min.

### Sodium 2-chloro-4-cyanobenzenesulfinate

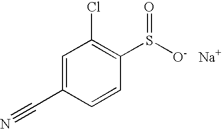

A solution of sodium sulfite (6.02 g, 47.74 mmol) in water (100 mL) was stirred at RT for 10 minutes. Sodium bicarbonate (8.02 g, 95.48 mmol) was added to the stirred solution. The resulting solution was stirred at 50° C. for 10 minutes. 2-Chloro-4-cyanobenzene-1-sulfonyl chloride (11.27 g, 47.74 mmol) was added dropwise to the solution and was stirred at 50° C. for 2 hours. The reaction mixture was evaporated to dryness and redissolved in ethanol (200 mL). The suspension was allowed to stir at RT for 20 minutes. The suspension was filtered and the filtrate evaporated to afford a white solid, this was stirred with acetonitrile (50 mL) and then filtered to afford the desired material as a white solid (10.0 g).

NMR Spectrum: ¹H NMR (400.132 MHz, DMSO-d₆) δ 7.80-7.77 (2H, m), 7.84 (1H, d)

The preparation of 2-chloro-4-(iodomethyl)-6-[(3S)-3-methylmorpholin-4-yl]pyrimidine was described earlier.

The preparation of phenyl N-[4-[4-[1-(2-chloro-4-cyanophenyl)sulfonylcyclopropyl]-6-[(3S)-3-methylmorpholin-4-yl]pyrimidin-2-yl]phenyl]carbamate is described below.

### Phenyl N-[4-[4-[1-(2-chloro-4-cyanophenyl)sulfonylcyclopropyl]-6-[(3S)-3-methylmorpholin-4-yl]pyrimidin-2-yl]phenyl]carbamate

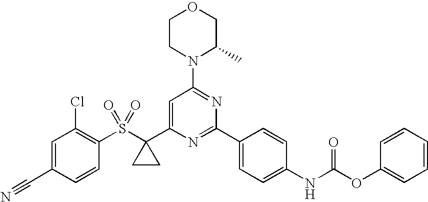

Sodium bicarbonate (1.400 g, 16.67 mmol) was added to 4-[1-[2-(4-aminophenyl)-6-[(3S)-3-methylmorpholin-4-yl]pyrimidin-4-yl]cyclopropyl]sulfonyl-3-chlorobenzonitrile (1.7 g, 3.33 mmol), in 1,4-dioxane (16.25 mL) and to the resulting suspension was added phenyl chloroformate (0.42 mL, 3.33 mmol) dropwise over 2 minutes. The reaction was stirred at RT for 2 hours then evaporated to dryness, the residue redissolved in DCM (20 mL), and washed with water (20 mL). The organic layer was dried over MgSO₄, filtered and evaporated to afford a solid which was triturated with diethyl ether to give the desired material as a cream solid (1.4 g).

NMR Spectrum: ¹H NMR (400.132 MHz, DMSO-d₆) δ 1.19 (3H, d), 1.76-1.80 (2H, m), 2.01-2.04 (2H, m), 3.14-3.19 (1H, m), 3.43-3.48 (1H, m), 3.59-3.62 (1H, m), 3.74 (1H, d), 3.94-3.97 (1H, m), 4.16 (1H, d), 4.49 (1H, s), 6.71 (1H, s), 7.24-7.30 (3H, m), 7.45 (2H, t), 7.53 (2H, d), 7.77 (2H, d), 7.90-7.92 (1H, m), 8.02 (1H, d), 8.35 (1H, d), 10.44 (1H, s)

LCMS Spectrum: m/z (ES+) (M+H)+=630; HPLC tR=3.02 min.

### 4-[1-[2-(4-Aminophenyl)-6-[(3S)-3-methylmorpholin-4-yl]pyrimidin-4-yl]cyclopropyl]sulfonyl-3-chlorobenzonitrile

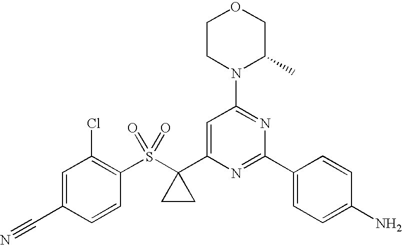

tert-Butyl N-[4-[4-[1-(2-chloro-4-cyanophenyl)sulfonylcyclopropyl]-6-[(3S)-3-methylmorpholin-4-yl]pyrimidin-2-yl]phenyl]carbamate (3.4 g, 5.57 mmol) was dissolved in methanol (17.86 mL) and to this was added 6 N hydrogen chloride in propan-2-ol (10 mL) and the reaction stirred for 2 hours at RT. The crude solution was triturated with diethyl ether to give the desired material as a white solid (3.50 g). The material was used without further purification.

MR Spectrum: ¹H NMR (400.132 MHz, DMSO-d₆) δ 1.20 (3H, d), 1.76-1.79 (2H, m), 2.01-2.04 (2H, m), 3.18-3.24 (1H, m), 3.42-3.48 (1H, m), 3.60 (1H, dd), 3.74-3.77 (1H, m), 3.94-3.98 (1H, m), 4.20 (1H, s), 4.51 (1H, s), 6.76 (1H, s), 7.11-7.14 (2H, m), 7.86 (2H, d), 7.95-7.97 (2H, m), 8.07 (1H, d), 8.37 (1H, d)

LCMS Spectrum: m/z (ES+) (M+H)+=510; HPLC tR=2.49 min.

### tert-Butyl N-[4-[4-l [-(2-chloro-4-cyanophenyl)sulfonylcyclopropyl]-6-[(3S)-3-methylmorpholin-4-yl]pyrimidin-2-yl]phenyl]carbamate

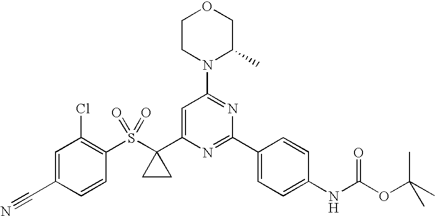

Sodium hydride (1.282 g, 26.71 mmol) was added rapidly to tert-butyl N-[4-[4-[(2-chloro-4-cyanophenyl)sulfonylmethyl]-6-[(3S)-3-methylmorpholin-4-yl]pyrimidin-2-yl]phenyl]carbamate (3.9 g, 6.68 mmol) in DMF (70 mL) and the mixture stirred at RT for 10 minutes before the slow addition 1,2-dibromoethane (2.302 mL, 26.71 mmol) in DMF (70 mL) at RT. The resulting suspension was stirred at RT for 90 minutes. A further portion of sodium hydride (1.0 eq) and 1,2 dibromoethane (1.0 eq) were rapidly added and the reaction was stirred for a further 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to give the desired material as a yellow solid (3.40 g).

NMR Spectrum: ¹H NMR (400.132 MHz, DMSO-d₆) δ 1.18 (3H, d), 1.50 (9H, s), 1.73-1.78 (2H, m), 1.99-2.05 (2H, m), 3.12-3.18 (1H, m), 3.45 (1H, t), 3.60 (1H, d), 3.74 (1H, d), 3.95 (1H, d), 4.15 (1H, d), 4.48 (1H, s), 6.68 (1H, s), 7.45 (2H, d), 7.69 (2H, d), 7.90 (1H, d), 8.01 (1H, d), 8.35 (1H, s), 9.52 (1H, s)

LCMS Spectrum: m/z (ES+) (M+H)+=610; HPLC tR=3.10 min.

### tert-Butyl N-[4-[4-[(2-chloro-4-cyanophenyl)sulfonylmethyl]-6-[(3S)-3-methylmorpholin-4-yl]pyrimidin-2-yl]phenyl]carbamate

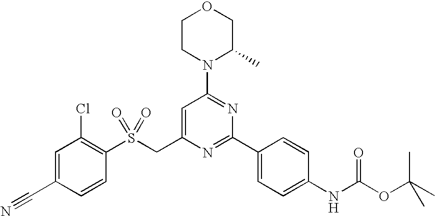

Sodium 2-chloro-4-cyanobenzenesulfinate (1.753 g, 7.84 mmol) and tert-butyl N-[4-[4-(iodomethyl)-6-[(3S)-3-methylmorpholin-4-yl]pyrimidin-2-yl]phenyl]carbamate (4 g, 7.84 mmol) were dissolved in DMF (50 mL) and stirred for 1 hour at RT. The solvent was evaporated to afford a yellow solid which was partitioned between water (50 mL) and DCM (75 mL). The layers were separated, the aqueous layer extracted with DCM (75 mL) and the combined organics concentrated in vacuo to give a yellow solid. This was rapidly stirred with ether (100 mL), to afford the desired material as an off white solid (3.90 g).

NMR Spectrum: ¹H NMR (400.132 MHz, DMSO-d₆) δ 1.21 (3H, d), 1.49 (9H, s), 3.19 (1H, dt), 3.48 (1H, dt), 3.63 (1H, dd), 3.76 (1H, d), 3.97 (1H, dd), 4.13 (1H, d), 4.41 (1H, s), 4.97 (2H, s), 6.79 (1H, s), 7.43 (2H, d), 7.61 (2H, d), 7.86 (1H, d), 7.91 (1H, dd), 8.53 (1H, d), 9.54 (1H, s)

LCMS Spectrum: m/z (ES+) (M+H)+=583.94; HPLC tR=3.07 min.

The preparation of tert-butyl N-[4-[4-(iodomethyl)-6-[(3S)-3-methylmorpholin-4-yl]pyrimidin-2-yl]phenyl]carbamate was described earlier.

### EXAMPLE 86

1-[4-[4-[1-(2,6-Difluorophenyl)sulfonylcyclopropyl]-6-[(3S)-3-methylmorpholin-4-yl]pyrimidin-2-,yl]phenyl]-3-methylurea

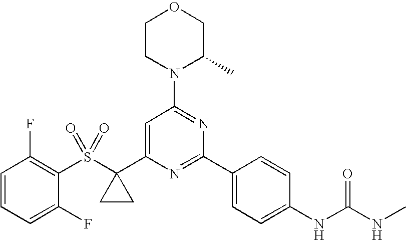

Phenyl N-[4-[4-[1-(2,6-difluorophenyl)sulfonylcyclopropyl]-6-[(3S)-3-methylmorpholin-4-yl]pyrimidin-2-yl]phenyl]carbamate (0.15 g, 0.25 mmol), triethylamine (0.103 mL, 0.74 mmol) and methylamine (0.74 mmol) were dissolved in dioxane (10 mL) and stirred at RT overnight. The reaction was evaporated to dryness and was purified by preparative HPLC, eluting with decreasingly polar mixtures of water (containing 1% ammonia) and acetonitrile, to give the desired material as a white solid.

NMR Spectrum: ¹H NMR (400.132 MHz, DMSO-d₆) δ 1.20 (3H, d), 1.69-1.67 (2H, m), 1.94-1.91 (2H, m), 2.66 (3H, d), 3.17 (1H, ddd), 3.47 (1H, ddd), 3.62 (1H, dd), 3.75 (1H, d), 3.96 (1H, dd), 4.16 (1H, d), 4.49 (1H, s), 6.04 (1H, q), 6.74 (1H, s), 7.25 (2H, t), 7.35 (2H, d), 7.80-7.73 (3H, m), 8.69 (1H, s);

LCMS Spectrum: m/z (ESI+) (M+H)+=544; HPLC tR=1.82 min

The following compounds were prepared in an analogous fashion from phenyl N-[4-[4-[1-(2,6-difluorophenyl)sulfonylcyclopropyl]-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 86a | 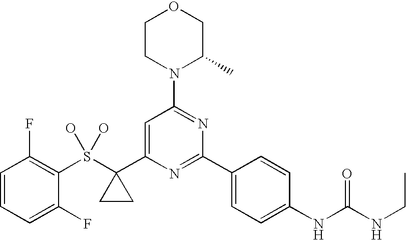 | 1-[4-[4-[1-(2,6-difluorophenyl)sulfonylcyclopropyl]-6-[(3S)-3-methylmorpholin-4-yl]pyrimidin-2-yl]phenyl]-3-ethylurea | 558 | 2.43 |
| 86b* | 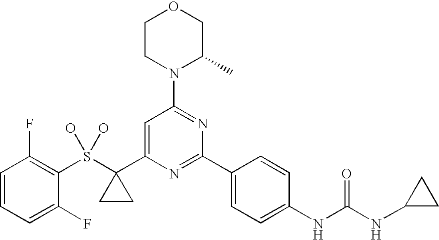 | 3-cyclopropyl-1-[4-[4-[1-(2,6-difluorophenyl)sulfonylcyclopropyl]-6-[(3S)-3-methylmorpholin-4-yl]pyrimidin-2-yl]phenyl]urea | 570 | 2.44 |
| 86c* | 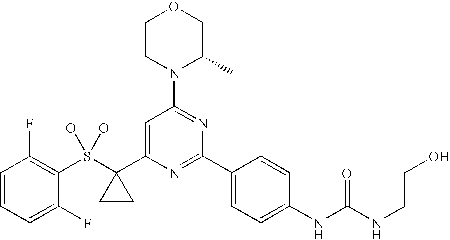 | 1-[4-[4-[1-(2,6-difluorophenyl)sulfonylcyclopropyl]-6-[(3S)-3-methylmorpholin-4-yl]pyrimidin-2-yl]phenyl]-3-(2-hydroxyethyl)urea | 574 | 2.07 |
| 86d* | 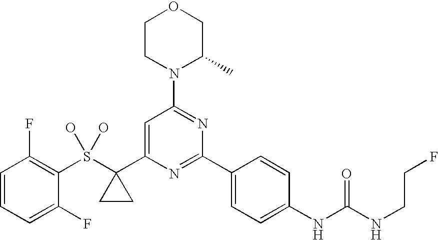 | 1-[4-[4-[1-(2,6-difluorophenyl)sulfonylcyclopropyl]-6-[(3S)-3-methylmorpholin-4-yl]pyrimidin-2-yl]phenyl]-3-(2-fluoroethyl)urea | 576 | 2.42 |
| 86e* | 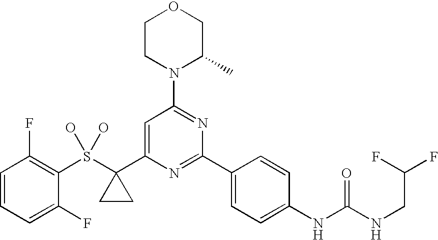 | 3-(2,2-difluoroethyl)-1-[4-[4-[1-(2,6-difluorophenyl)sulfonylcyclopropyl]-6-[(3S)-3-methylmorpholin-4-yl]pyrimidin-2-yl]phenyl]urea | 594 | 2.55 |
| 86f* | 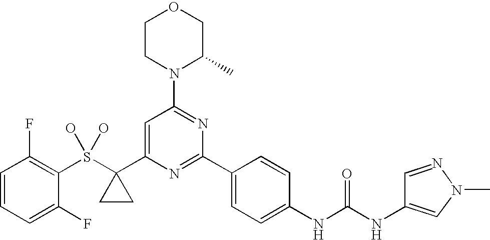 | 1-[4-[4-[1-(2,6-difluorophenyl)sulfonylcyclopropyl]-6-[(3S)-3-methylmorpholin-4-yl]pyrimidin-2-yl]phenyl]-3-(1-methylpyrazol-4-yl)urea | 610 | 2.28 |

### EXAMPLE 86a

¹H NMR (400.132 MHz, DMSO-d₆) δ 1.06 (3H, t), 1.20 (3H, d), 1.71-1.65 (2H, m), 1.93-1.89 (2H, m), 3.20-3.09 (3H, m), 3.47 (1H, ddd), 3.62 (1H, dd), 3.75 (1H, d), 3.96 (2H, ddd), 4.48 (1H, s), 6.14 (1H, t), 6.74 (1H, s), 7.25 (2H, t), 7.35 (2H, d), 7.80-7.73 (3H, m), 8.61 (1H, s);

### EXAMPLE 86b

¹H NMR (400.132 MHz, DMSO-d₆) δ 0.43-0.39 (2H, m), 0.67-0.62 (2H, m), 1.20 (3H, d), 1.71-1.65 (2H, m), 1.92-1.91 (2H, m), 2.57-2.54 (1H, m), 3.17 (1H, ddd), 3.47 (1H, ddd), 3.62 (1H, dd), 3.75 (1H, d), 3.96 (1H, dd), 4.16 (1H, d), 4.48 (1H, s), 6.40 (1H, s), 6.75 (1H, s), 7.25 (2H, t), 7.36 (2H, d), 7.80-7.73 (3H, m), 8.49 (1H, s);

### EXAMPLE 86c

¹H NMR (400.132 MHz, DMSO-d₆) δ 1.20 (3H, d), 1.71-1.65 (2H, m), 1.94-1.88 (2H, m), 3.21-3.14 (3H, m), 3.50-3.44 (3H, m), 3.62 (1H, dd), 3.75 (1H, d), 3.96 (1H, dd), 4.16 (1H, d), 4.48 (1H, s), 4.72 (1H, t), 6.23 (1H, t), 6.74 (1H, s), 7.25 (2H, t), 7.34 (2H, d), 7.80-7.73 (3H, m), 8.75 (1H, s);

### EXAMPLE 86d

¹H NMR (400.132 MHz, DMSO-d₆) δ 1.20 (3H, d), 1.69-1.67 (2H, m), 1.93-1.89 (2H, m), 3.17 (1H, ddd), 3.50-3.36 (3H, m), 3.62 (1H, dd), 3.75 (1H, d), 3.96 (1H, dd), 4.16 (1H, d), 4.47 (2H, tt), 4.49 (1H, s), 6.41 (1H, t), 6.75 (1H, s), 7.25 (2H, t), 7.36 (2H, d), 7.80-7.74 (3H, m), 8.76 (1H, s);

### EXAMPLE 86e

¹H NMR (400.132 MHz, DMSO-d₆) δ 1.20 (3H, d), 1.69-1.67 (2H, m), 1.93-1.89 (2H, m), 3.17 (1H, ddd), 3.59-3.44 (3H, m), 3.62 (1H, dd), 3.75 (1H, d), 3.96 (1H, dd), 4.16 (1H, d), 4.48 (1H, s), 6.07 (1H, tt), 6.51 (1H, t), 6.75 (1H, s), 7.25 (2H, t), 3.37 (2H, d), 7.80-7.72 (3H, m), 8.87 (1H, s);

### EXAMPLE 86f

¹H NMR (400.132 MHz, DMSO-d₆) δ 1.20 (3H, d), 1.70-1.68 (2H, m), 1.94-1.89 (2H, m), 3.18 (1H, ddd), 3.47 (1H, ddd), 3.63 (1H, dd), 3.76 (1H, d), 3.79 (3H, s), 3.97 (1H, dd), 4.16 (1H, d), 4.49 (1H, s), 6.76 (1H, s), 7.26 (2H, t), 7.41-7.38 (3H, m), 7.80-7.73 (4H, m), 8.36 (1H, s), 8.79 (1H, s).

The preparation of phenyl N-[4-[4-[1-(2,6-difluorophenyl)sulfonylcyclopropyl]-6-[(3S)-3-methylmorpholin-4-yl]pyrimidin-2-yl]phenyl]carbamate is described below.

### Phenyl N-[4-[4-[1-(2,6-difluorophenyl)sulfonylcyclopropyl]-6-[(3S)-3-methylmorpholin-4-yl]pyrimidin-2-yl]phenyl]carbamate

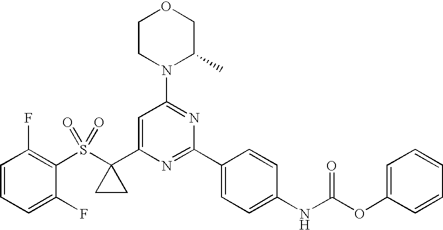

4-[4-[1-(2,6-Difluorophenyl)sulfonylcyclopropyl]-6-[(3S)-3-methylmorpholin-4-yl]pyrimidin-2-yl]aniline (as the hydrochloride salt) (1.67 g, 3.19 mmol) and sodium bicarbonate (2.68 g, 31.93 mmol) were added to DCM (60 mL) and stirred for 10 minutes. Phenyl chloroformate (0.521 mL, 4.15 mmol) was added slowly and the reaction stirred for 1 hour. The reaction mixture was quenched with a saturated aqueous solution of ammonium chloride (50 mL), extracted with ethyl acetate (3×50 mL), the organic layer was dried over MgSO₄, filtered and evaporated to afford an orange solid. The crude product was purified by flash silica chromatography, elution gradient 30 to 60% ethyl acetate in isohexane, to give the desired material as a yellow solid (1.4 g).

NMR Spectrum: ¹H NMR (400.132 MHz, DMSO-d₆) δ 1.21 (3H, d), 1.72-1.66 (2H, m), 1.93-1.92 (2H, m), 3.18 (1H, ddd), 3.47 (1H, ddd), 3.62 (1H, dd), 3.76 (1H, d), 3.96 (1H, dd), 4.18 (1H, d), 4.50 (1H, s), 6.79 (1H, s), 7.30-7.23 (5H, m), 7.45 (2H, t), 7.49 (2H, d), 7.79-7.72 (1H, m), 7.84 (2H, d), 10.38 (1H, s).

LCMS Spectrum: m/z (ESI+) (M+H)+=607; HPLC tR=3.02 min.

### 4-[4-l [-(2,6-Difluorophenyl)sulfonylcyclopropyl]-6-[(3S)-3-methylmorpholin-4-yl]pyrimidin-2-yl]aniline

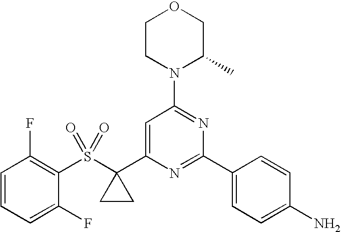

tert-Butyl N-[4-[4-[1-(2,6-difluorophenyl)sulfonylcyclopropyl]-6-[(3S)-3-methylmorpholin-4-yl]pyrimidin-2-yl]phenyl]carbamate (2.15 g, 3.66 mmol) was added to 6.0 N hydrogen chloride in propan-2-ol (30 mL) and stirred for 2 hours at RT. The crude solution was triturated with diethyl ether to give the desired material (as the hydrochloride salt) as a white solid (1.67 g).

NMR Spectrum: ¹H NMR (400.132 MHz, DMSO-d₆) δ 1.04 (3H, d), 1.76-1.72 (2H, m), 1.98-1.90 (2H, m), 3.24 (1H, ddd), 3.46 (1H, ddd), 3.60 (1H, dd), 3.76 (1H, d), 3.97 (1H, dd), 4.25 (1H, s), 4.53 (1H, s), 6.85 (1H, s), 7.15-7.02 (2H, m), 7.29 (2H, t), 7.84-7.77 (1H, m), 7.91 (2H, d); NH2 not visible)

LCMS Spectrum: m/z (ESI+) (M+H)+=487; HPLC tR=2.45 min.

### tert-Butyl N-[4-[4-[1-(2,6-difluorophenyl)sulfonylcyclopropyl]-6-[(3S)-3-methylmorpholin-4-yl]pyrimidin-2-yl]phenyl]carbamate

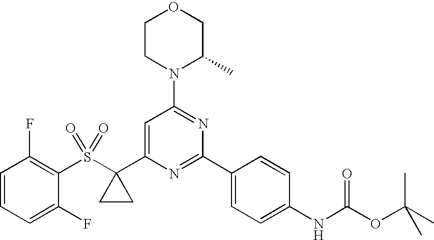

Sodium hydride (1.027 g, 21.41 mmol) was added rapidly to tert-butyl N-[4-[4-[(2,6-difluorophenyl)sulfonylmethyl]-6-[(3S)-3-methylmorpholin-4-yl]pyrimidin-2-yl]phenyl]carbamate (3.0 g, 5.35 mmol) in DMF (70 mL) and stirred at RT for 10 minutes before the slow addition 1,2-dibromoethane (1.845 mL, 21.41 mmol) in DMF (70 mL). The resulting suspension was stirred at RT for 1.5 hours. A further portion of sodium hydride (2.0 eq) and 1,2 dibromoethane (2.0 eq) were rapidly added and the reaction was stirred for a further 30 minutes. The reaction mixture was quenched with water (50 mL), extracted with ethyl acetate (3×50 mL), the organic layer was dried over MgSO₄, filtered and evaporated to afford brown gum. The crude product was purified by flash silica chromatography, elution gradient 20 to 50% ethyl acetate in isohexane, to afford a yellow foam. This was dissolved in 40% ethyl acetate in isohexane and upon stirring the desired material precipitated as a white solid (2.15 g).

NMR Spectrum: ¹H NMR (400.132 MHz, DMSO-d₆) δ 1.20 (3H, d), 1.49 (9H, s), 1.71-1.65 (2H, m), 1.93-1.90 (2H, m), 3.17 (1H, ddd), 3.47 (1H, ddd), 3.62 (1H, dd), 3.75 (1H, d), 3.96 (1H, dd), 4.16 (1H, d), 4.48 (1H, s), 6.76 (1H, s), 7.25 (2H, t), 7.42 (2H, d), 7.80-7.72 (3H, m), 9.49 (1H, s);

LCMS Spectrum: m/z (ESI+) (M+H)+=587; HPLC tR=3.12 min.

### tert-Butyl N-[4-[4-[(2,6-difluorophenyl)sulfonylmethyl]-6-[(3S)-3-methylmorpholin-4-yl]pyrimidin-2-yl]phenyl]carbamate

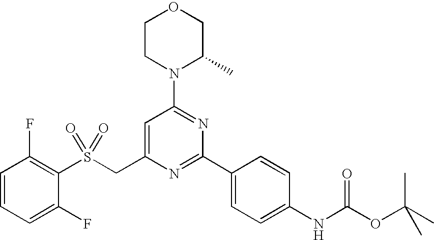

Sodium 2,6-difluorobenzenesulfonate (1.270 g, 5.88 mmol) and tert-butyl N-[4-[4-(iodomethyl)-6-[(3S)-3-methylmorpholin-4-yl]pyrimidin-2-yl]phenyl]carbamate (3 g, 5.88 mmol) were dissolved in DMF (50 mL) and stirred for 1 hour at RT. The solvent was evaporated to afford a yellow solid which was partitioned between water (50 mL) and DCM (75 mL). The layers were separated, the aqueous layer extracted with DCM (75 mL) and the 20 combined organics concentrated in vacuo to give a yellow solid. The solid was rapidly stirred with ether (100 mL), to afford a solid which was collected by filtration and dried under vacuum to give the desired material as an off white solid (3.0 g).

NMR Spectrum: ¹H NMR (400.132 MHz, DMSO-d₆) δ 1.22 (3H, d), 1.49 (9H, s), 3.20 (1H, ddd), 3.49 (1H, ddd), 3.64 (1H, dd), 3.77 (1H, d), 3.98 (1H, dd), 4.14 (1H, d), 4.42 (1H, s), 4.78 (2H, s), 6.81 (1H, s), 7.32 (2H, t), 7.42 (2H, d), 7.73 (2H, d), 7.85-7.78 (1H, m) 9.50 (1H, s);

LCMS Spectrum: m/z (ESI+) (M+H)+=561; HPLC tR=2.84 min.

The preparation of tert-butyl N-[4-[4-(iodomethyl)-6-[(3S)-3-methylmorpholin-4-yl]pyrimidin-2-yl]phenyl]carbamate was described earlier.

### EXAMPLE 87

### 1-[4-[4-(1-Ethylsulfonylcyclobutyl)-6-[(3S)-3-methylmorpholin-4-yl]pyrimidin-2-yl]phenyl]-3-(2-hydroxyethyl)urea

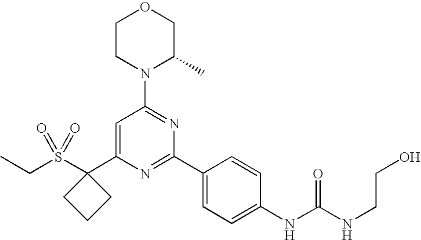

Ethanolamine (0.023 mL, 0.37 mmol) was added in one portion to phenyl N-[4-[4-(1-ethylsulfonylcyclobutyl)-6-[(3S)-3-methylmorpholin-4-yl]pyrimidin-2-yl]phenyl]carbamate (101 mg, 0.19 mmol) and triethylamine (0.078 mL, 0.56 mmol) in NMP (2 mL) at RT and stirred for a period of 16 hours. The crude product was purified by preparative HPLC, eluting with decreasingly polar mixtures of water (containing 1% ammonia) and acetonitrile, to afford desired material as a cream solid (71 mg).

NMR Spectrum: ¹H NMR (399.902 MHz, DMSO-d₆) δ 1.15 (t, 3H), 1.23 (d, 3H), 1.86-1.97 (m, 1H), 2.01-2.11 (m, 1H), 2.77-2.87 (m, 2H), 2.90-2.99 (m, 4H), 3.16-3.25 (m, 2H), 3.46 (q, 1H), 3.52 (dd, 3H), 3.65 (dd, 1H), 3.77 (d, 1H), 3.98 (dd, 1H), 4.20-4.28 (m, 1H), 4.52-4.61 (m, 1H), 4.77 (t, 1H), 6.28 (t, 1H), 6.72 (s, 1H), 7.50 (d, 2H), 8.22 (d, 2H), 8.84 (s, 1H)

LCMS Spectrum: m/z (ESI+) (M+H)+=504; HPLC tR=1.79 min.

The compounds below were prepared in an analogous fashion from phenyl N-[4-[4-(1-ethylsulfonylcyclobutyl)-6-[(3S)-3-methylmorpholin-4-yl]pyrimidin-2-yl]phenyl]carbamate and the appropriate amine.

| | | | LCMS | Retention |
|---|---|---|---|---|
| Example | Structure | NAME | MH+ | time (min) |
| 87a | 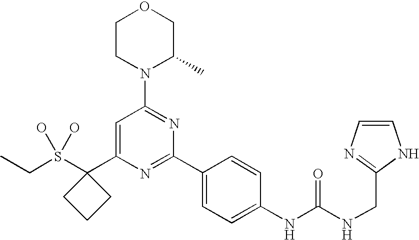 | 1-[4-[4-(1-ethylsulfonylcyclobutyl)-6-[(3S)-3-methylmorpholin-4-yl]pyrimidin-2-yl]phenyl]-3-(1H-imidazol-2-ylmethyl)urea | 540 | 1.87 |
| 87b | 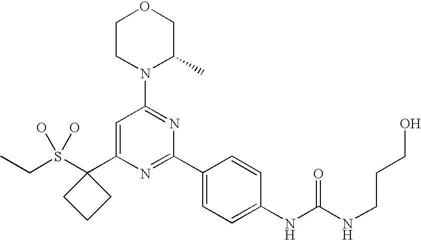 | 1-[4-[4-(1-ethylsulfonylcyclobutyl)-6-[(3S)-3-methylmorpholin-4-yl]pyrimidin-2-yl]phenyl]-3-(3-hydroxypropyl)urea | 518 | 1.83 |
| 87c | 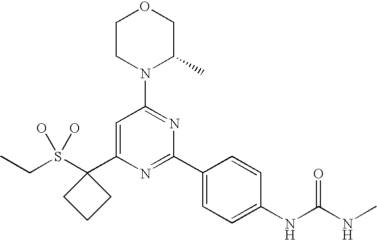 | 1-[4-[4-(1-ethylsulfonylcyclobutyl)-6-[(3S)-3-methylmorpholin-4-yl]pyrimidin-2-yl]phenyl]-3-methylurea | 474 | 1.95 |
| 87d | 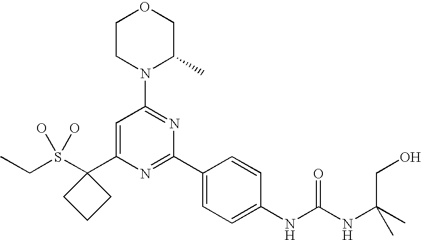 | 1-[4-[4-(1-ethylsulfonylcyclobutyl)-6-[(3S)-3-methylmorpholin-4-yl]pyrimidin-2-yl]phenyl]-3-(1-hydroxy-2-methylpropan-2-yl)urea | 532 | 2.08 |
| 87e | 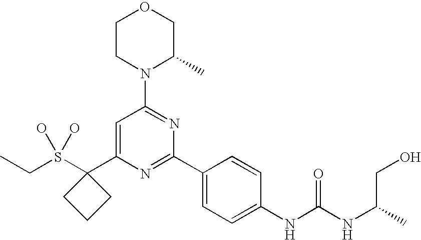 | 1-[4-[4-(1-ethylsulfonylcyclobutyl)-6-[(3S)-3-methylmorpholin-4-yl]pyrimidin-2-yl]phenyl]-3-[(2S)-1-hydroxypropan-2-yl]urea | 518 | 1.88 |
| 87f | 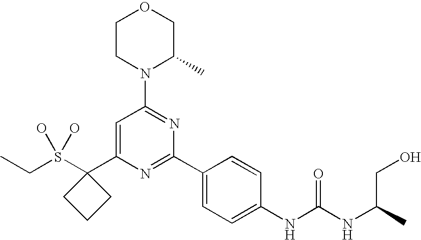 | 1-[4-[4-(1-ethylsulfonylcyclobutyl)-6-[(3S)-3-methylmorpholin-4-yl]pyrimidin-2-yl]phenyl]-3-[(2R)-1-hydroxypropan-2-yl]urea | 518 | 1.89 |

### EXAMPLE 87a

¹H NMR(399.902 MHz, DMSO-d₆) δ 1.15 (t, 3H), 1.23 (d, 3H), 1.87-1.97 (m, 1H), 2.02-2.10 (m, 1H), 2.77-2.87 (m, 2H), 2.90-3.01 (m, 4H), 3.17-3.25 (m, 1H), 3.50 (td, 1H), 3.65 (dd, 1H), 3.77 (d, 1H), 3.98 (dd, 1H), 4.21-4.29 (m, 1H), 4.33 (d, 2H), 4.52-4.61 (m, 1H), 6.65 (t, 1H), 6.73 (s, 1H), 6.82-6.86 (m, 1H), 7.02-7.07 (m, 1H), 7.52 (d, 2H), 8.24 (d, 2H), 8.97 (s, 1H), 11.87 (s, 1H)

### EXAMPLE 87b

¹H NMR (399.902 MHz, DMSO-d₆) δ 1.15 (t, 3H), 1.23 (d, 3H), 1.60 (quintet, 2H), 1.86-1.97 (m, 1H), 2.01-2.11 (m, 1H), 2.77-2.87 (m, 2H), 2.90-2.99 (m, 4H), 3.14-3.25 (m, 3H), 3.47 (q, 2H), 3.49-3.54 (m, 1H), 3.65 (dd, 1H), 3.77 (d, 1H), 3.98 (dd, 1H), 4.20-4.30 (m, 1H), 4.51-4.61 (m, 1H), 4.52 (t, 1H), 6.23 (t, 1H), 6.72 (s, 1H), 7.50 (d, 2H), 8.22 (d, 2H), 8.75 (s, 1H)

### EXAMPLE 87c

¹H NMR (399.902 MHz, DMSO-d₆) δ 1.15 (t, 3H), 1.23 (d, 3H), 1.85-1.97 (m, 1H), 2.00-2.10 (m, 1H), 2.66 (d, 3H), 2.77-2.87 (m, 2H), 2.90-3.00 (m, 4H), 3.21 (td, 1H), 3.50 (td, 1H), 3.65 (dd, 1H), 3.77 (d, 1H), 3.98 (dd, 1H), 4.20-4.29 (m, 1H), 4.52-4.60 (m, 1H), 6.09 (q, 1H), 6.72 (s, 1H), 7.51 (d, 2H), 8.22 (d, 2H), 8.79 (s, 1H)

### EXAMPLE 87d

¹H NMR (399.902 MHz, DMSO-d₆) δ 1.15 (t, 3H), 1.23 (d, 3H), 1.24 (s, 6H), 1.86-1.97 (m, 1H), 2.01-2.11 (m, 1H), 2.76-2.87 (m, 2H), 2.90-3.01 (m, 4H), 3.22 (dd, 1H), 3.39 (d, 2H), 3.50 (td, 1H), 3.65 (dd, 1H), 3.77 (d, 1H), 3.98 (dd, 1H), 4.19-4.30 (m, 1H), 4.51-4.61 (m, 1H), 5.00 (t, 1H), 6.02 (s, 1H), 6.72 (s, 1H), 7.46 (d, 2H), 8.21 (d, 2H), 8.77 (s, 1H)

### EXAMPLE 87e

¹H NMR (399.902 MHz, DMSO-d₆) δ 1.09 (d, 3H), 1.15 (t, 3H), 1.23 (d, 3H), 1.86-1.97 (m, 1H), 2.01-2.11 (m, 1H), 2.77-2.87 (m, 2H), 2.90-3.00 (m, 4H), 3.21 (td, 1H), 3.32-3.42 (m, 2H), 3.50 (td, 1H), 3.65 (dd, 1H), 3.68-3.74 (m, 1H), 3.77 (d, 1H), 3.98 (dd, 1H), 4.20-4.29 (m, 1H), 4.52-4.61 (m, 1H), 4.83 (t, 1H), 6.12 (d, 1H), 6.72 (s, 1H), 7.49 (d, 2H), 8.22 (d, 2H), 8.75 (s, 1H)

### EXAMPLE 87f

¹H NMR (399.902 MHz, DMSO-d₆) δ 1.08 (d, 3H), 1.15 (t, 3H), 1.23 (d, 3H), 1.86-1.97 (m, 1H), 2.01-2.11 (m, 1H), 2.77-2.87 (m, 2H), 2.90-3.00 (m, 4H), 3.21 (td, 1H), 3.32-3.42 (m, 2H), 3.50 (td, 1H), 3.65 (dd, 1H), 3.68-3.74 (m, 1H), 3.76-3.79 (m, 1H), 3.98 (dd, 1H), 4.20-4.29 (m, 1H), 4.52-4.61 (m, 1H), 4.83 (t, 1H), 6.12 (d, 1H), 6.72 (s, 1H), 7.48 (d, 2H), 8.22 (d, 2H), 8.75 (s, 1H)

The preparation of phenyl N-[4-[4-(1-ethylsulfonylcyclobutyl)-6-[(3S)-3-methylmorpholin-4-yl]pyrimidin-2-yl]phenyl]carbamate is described below:

### Phenyl N-[4-[4-(1-ethylsulfonylcyclobutyl)-6-[(3S)-3-methylmorpholin-4-yl]pyrimidin-2-yl]phenyl]carbamate

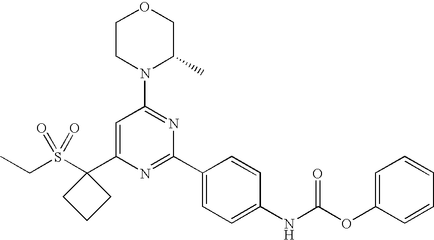

Phenyl chloroformate (0.211 mL, 1.68 mmol) was added dropwise to 4-[4-(1-ethylsulfonylcyclobutyl)-6-[(3S)-3-methylmorpholin-4-yl]pyrimidin-2-yl]aniline (700 mg, 1.68 mmol) and sodium hydrogen carbonate (141 mg, 1.68 mmol) in dioxane (20 mL) and the resulting suspension stirred at RT for 3 hours. The reaction mixture was evaporated and DCM (50 mL) added and washed sequentially with water (20 mL) and saturated brine (20 mL). The organic layer was dried over MgSO₄, filtered and evaporated to afford desired material as a yellow oil which solidified on standing. (930 mg)

NMR Spectrum: ¹H NMR (399.902 MHz, DMSO-d₆) δ 1.16 (t, 3H), 1.25 (d, 3H), 1.87-1.98 (m, 1H), 2.02-2.12 (m, 1H), 2.78-2.88 (m, 2H), 2.92-3.00 (m, 4H), 3.23 (td, 1H), 3.48-3.55 (m, 1H), 3.66 (dd, 1H), 3.78 (d, 1H), 3.99 (dd, 1H), 4.21-4.32 (m, 1H), 4.53-4.64 (m, 1H), 6.76 (s, 1H), 7.24-7.32 (m, 3H), 7.46 (dt, 2H), 7.64 (d, 2H), 8.32 (d, 2H), 10.44 (s, 1H)

LCMS Spectrum: m/z (ESI+) (M+H)+=537.15; HPLC tR=2.95 min.

### 4-[4-(1-Ethylsulfonylcyclobutyl)-6-[(3S)-3-methylmorpholin-4-yl]pyrimidin-2-yl]aniline

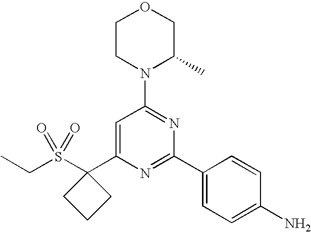

Bis(triphenylphosphine)palladium(II) chloride (0.137 g, 0.19 mmol) was added in one portion to 2-chloro-4-(1-ethylsulfonylcyclobutyl)-6-[(3S)-3-methylmorpholin-4-yl]pyrimidine (1.4 g, 3.89 mmol), 4-(4,4,5,5-tetramethyl-1,3,2-dioxaborolan-2-yl)aniline (0.852 g, 3.89 mmol) and sodium carbonate (9.73 ml, 19.45 mmol) in a DMF/DME/water/ethanol solution at RT under nitrogen. The reaction mixture was thoroughly degassed and was stirred at 80° C. for 2 hours. The reaction mixture was evaporated to dryness and redissolved in DCM (50 mL), and washed sequentially with water (50 mL) and saturated brine (50 mL). The organic layer was dried over MgSO₄, filtered and evaporated to afford crude product. The crude product was purified by flash silica chromatography, elution gradient 30 to 100% ethyl acetate in DCM, to 25 afford desired material as a yellow dry film (1.536 g).

NMR Spectrum: ¹H NMR (399.902 MHz, DMSO-d₆) δ 1.15 (t, 3H), 1.22 (d, 3H), 1.85-1.96 (m, 1H), 2.02-2.11 (m, 1H), 2.75-2.86 (m, 2H), 2.89-2.99 (m, 4H), 3.19 (td, 1H), 3.50 (td, 1H), 3.64 (dd, 1H), 3.76 (d, 1H), 3.97 (dd, 2H), 4.16-4.25 (m, 1H), 5.56 (s, 2H), 6.59-6.63 (m, 3H), 8.06 (d, 2H)

LCMS Spectrum: m/z (ESI+) (M+H)+=417.25; HPLC tR=2.03 min.

### 2-Chloro-4-(1-ethylsulfonylcyclobutyl)-6-[(3S)-3-methylmorpholin-4-yl]pyrimidine

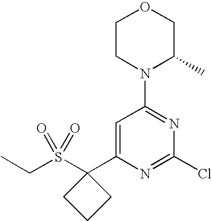

Sodium hydroxide (50% w/w solution) (27.4 g, 683.96 mmol) was added to 2-chloro-4-(ethylsulfonylmethyl)-6-[(3S)-3-methylmorpholin-4-yl]pyrimidine (3.977 g, 12.44 mmol), 1,3-dibromopropane (3.79 mL, 37.31 mmol) and tetrabutylammonium bromide (0.401 g, 1.24 mmol) in toluene (200 mL) and the resulting suspension stirred at 45° C. for 3 hours. The organics were washed with water twice, dried over MgSO₄, filtered and evaporated. The crude product was purified by flash silica chromatography, elution gradient 30 to 50% ethyl acetate in DCM, to afford desired material as a colourless dry film (1.47 g).

NMR Spectrum: ¹H NMR (399.902 MHz, DMSO-d₆) δ 1.13-1.19 (m, 3H), 1.22 (d, 3H), 1.84-1.95 (m, 1H), 1.98-2.10 (m, 1H), 2.65-2.77 (m, 2H), 2.82-2.92 (m, 2H), 2.96 (q, 2H), 3.18-3.25 (m, 1H), 3.45 (td, 1H), 3.60 (dd, 1H), 3.73 (d, 1H), 3.94 (dd, 1H), 3.99-4.12 (m, 1H), 4.36-4.50 (m, 1H), 6.83 (s, 1H)

LCMS Spectrum: m/z (ESI+) (M+H)+=360.22; HPLC tR=2.13 min.

The preparation of 2-chloro-4-(ethylsulfonylmethyl)-6-[(3S)-3-methylmorpholin-4-yl]pyrimidine was described earlier.

### EXAMPLE 88

### 1-[4-[4-(1-Ethylsulfonylcyclobutyl)-6-[(3S)-3-methylmorpholin-4-yl]pyrimidin-2-yl]phenyl]-3-(3-hydroxypropyl)thiourea

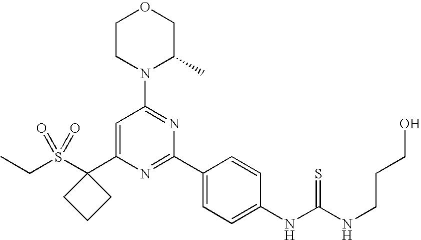

A solution of 1,1′-thiocarbonyldiimidazole (55.6 mg, 0.31 mmol) in DCM (2 mL) was added to a stirred solution of 4-[4-(1-ethylsulfonylcyclobutyl)-6-[(3S)-3-methylmorpholin-4-yl]pyrimidin-2-yl]aniline (100 mg, 0.24 mmol) in THF (1 mL) and DCM (2 mL) at RT, over a period of 2 minutes under nitrogen. The resulting solution was stirred at RT for 2 hours. Triethylamine (0.033 mL, 0.24 mmol) and 3-amino-1-propanol (0.092 mL, 1.20 mmol) were added to the reaction mixture. The resulting solution was stirred at RT for 60 hours. The reaction mixture was evaporated to dryness and redissolved in acetonitrile (2 mL), filtered and purified by preparative HPLC, eluting with decreasingly polar mixtures of water (containing 1% ammonia) and acetonitrile, to dryness to afford desired material as a beige solid. (100 mg)

NMR Spectrum: ¹H NMR (399.902 MHz, DMSO-d₆) δ 1.15 (t, 3H), 1.24 (d, 3H), 1.72 (quintet, 2H), 1.86-1.97 (m, 1H), 2.03-2.12 (m, 1H), 2.77-2.88 (m, 2H), 2.91-3.00 (m, 4H), 3.18-3.26 (m, 1H), 3.45-3.59 (m, 5H), 3.65 (dd, 1H), 3.77 (d, 1H), 3.98 (dd, 1H), 4.19-4.29 (m, 1H), 4.49-4.63 (m, 2H), 6.75 (s, 1H), 7.57 (d, 2H), 7.86-7.94 (m, 1H), 8.28 (d, 2H), 9.65-9.74 (m, 1H)

LCMS Spectrum: m/z (ES+) (M+H)+=534.8; HPLC tR=2.16 min.

The compounds below were prepared in an analogous fashion from either 4-[4-(1-ethylsulfonylcyclobutyl)-6-[(3S)-3-methylmorpholin-4-yl]pyrimidin-2-yl]aniline or 4-[4-(1-ethylsulfonylcyclopropyl)-6-[(3S)-3-methylmorpholin-4-yl]pyrimidin-2-yl]aniline and the appropriate amine.

| | | | LCMS | Retention |
|---|---|---|---|---|
| Example | Structure | NAME | MH+ | time (min) |
| 88a | 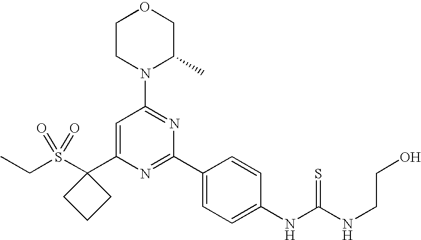 | 1-[4-[4-(1-ethylsulfonylcyclobutyl)-6-[(3S)-3-methylmorpholin-4-yl]pyrimidin-2-yl]phenyl]-3-(2-hydroxyethyl)thiourea | 521 | 2.13 |
| 88b | 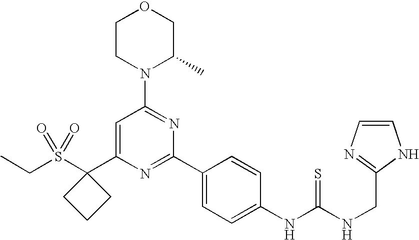 | 1-[4-[4-(1-ethylsulfonylcyclobutyl)-6-[(3S)-3-methylmorpholin-4-yl]pyrimidin-2-yl]phenyl]-3-(1H-imidazol-2-ylmethyl)thiourea | 557 | 1.51 |
| 88c | 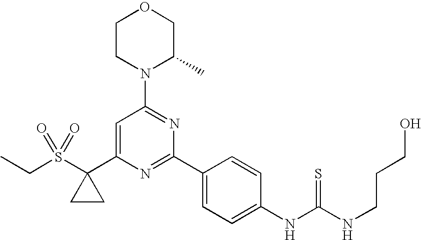 | 1-[4-[4-(1-ethylsulfonylcyclopropyl)-6-[(3S)-3-methylmorpholin-4-yl]pyrimidin-2-yl]phenyl]-3-(3-hydroxypropyl)thiourea | 5.21 | 2.33 |
| 88d | 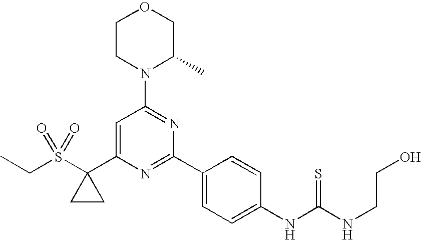 | 1-[4-[4-(1-ethylsulfonylcyclopropyl)-6-[(3S)-3-methylmorpholin-4-yl]pyrimidin-2-yl]phenyl]-3-(2-hydroxyethyl)thiourea | 507 | 2.30 |
| 88e | 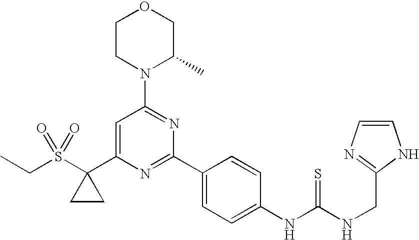 | 1-[4-[4-(1-ethylsulfonylcyclopropyl)-6-[(3S)-3-methylmorpholin-4-yl]pyrimidin-2-yl]phenyl]-3-(1H-imidazol-2-ylmethyl)thiourea | 543 | 2.36 |

### EXAMPLE 88a

¹H NMR (399.902 MHz, DMSO-d₆) δ 1.15 (t, 3H), 1.24 (d, 3H), 1.86-1.97 (m, 1H), 2.03-2.11 (m, 1H), 2.77-2.88 (m, 2H), 2.91-3.00 (m, 4H), 3.18-3.27 (m, 1H), 3.51 (td, 1H), 3.55-3.60 (m, 4H), 3.65 (dd, 1H), 3.77 (d, 1H), 3.98 (dd, 1H), 4.18-4.30 (m, 1H), 4.54-4.63 (m, 1H), 4.77-4.86 (m, 1H), 6.75 (s, 1H), 7.62 (d, 2H), 7.84-7.90 (m, 1H), 8.28 (d, 2H), 9.81 (s, 1H)

### EXAMPLE 88b

¹H NMR (399.902 MHz, DMSO-d₆) δ 1.15 (t, 3H), 1.24 (d, 3H), 1.86-1.97 (m, 1H), 2.02-2.12 (m, 1H), 2.78-2.88 (m, 2H), 2.91-3.00 (m, 4H), 3.18-3.26 (m, 1H), 3.51 (td, 1H), 3.66 (dd, 1H), 3.77 (d, 1H), 3.98 (dd, 1H), 4.19-4.30 (m, 1H), 4.53-4.63 (m, 1H), 4.71 (d, 2H), 6.75 (s, 1H), 6.99 (s, 2H), 7.69 (d, 2H), 8.18-8.25 (m, 1H), 8.30 (d, 2H), 9.98-10.09 (m, 1H), 11.84-12.09 (m, 1H)

### EXAMPLE 88c

¹H NMR (399.902 MHz, DMSO-d₆) δ 1.24 (d, 3H), 1.33 (t, 3H), 1.54-1.59 (m, 2H), 1.61-1.66 (m, 2H), 1.72 (quintet, 2H), 3.22 (td, 1H), 3.40-3.59 (m, 7H), 3.64 (dd, 1H), 3.77 (d, 1H), 3.98 (dd, 1H), 4.16-4.28 (m, 1H), 4.52-4.64 (m, 2H), 6.83 (s, 1H), 7.58 (d, 2H), 7.90-7.97 (m, 1H), 8.25 (d, 2H), 9.74 (s, 1H)

### EXAMPLE 88d

¹H NMR (399.902 MHz, DMSO-d₆) δ 1.24 (d, 3H), 1.33 (t, 3H), 1.55-1.59 (m, 2H), 1.61-1.66 (m, 2H), 3.22 (td, 1H), 3.44 (q, 2H), 3.45-3.52 (m, 1H), 3.55-3.60 (m, 4H), 3.64 (dd, 1H), 3.77 (d, 1H), 3.98 (dd, 1H), 4.15-4.28 (m, 1H), 4.52-4.63 (m, 1H), 4.82-4.90 (m, 1H), 6.82 (s, 1H), 7.63 (d, 2H), 7.88-7.93 (m, 1H), 8.25 (d, 2H), 9.85 (s, 1H)

### EXAMPLE 88e

¹H NMR (399.902 MHz, DMSO-d₆) δ 1.24 (d, 3H), 1.33 (t, 3H), 1.54-1.59 (m, 2H), 1.61-1.66 (m, 2H), 3.22 (td, 1H), 3.45 (q, 2H), 3.45-3.52 (m, 1H), 3.64 (dd, 1H), 3.77 (d, 1H), 3.98 (dd, 1H), 4.15-4.28 (m, 1H), 4.53-4.63 (m, 1H), 4.69-4.74 (m, 2H), 6.83 (s, 1H), 6.86-7.15 (m, 2H), 7.70 (d, 2H), 8.22-8.29 (m, 1H), 8.27 (d, 2H), 10.05 (s, 1H), 11.95 (s, 1H)

The preparation of both 4-[4-(1-ethylsulfonylcyclobutyl)-6-[(3S)-3-methylmorpholin-4-yl]pyrimidin-2-yl]aniline and 4-[4-(1-ethylsulfonylcyclopropyl)-6-[(3S)-3-methylmorpholin-4-yl]pyrimidin-2-yl]aniline were described earlier.

### EXAMPLE 89

### 4-[6-[1-(Benzenesulfonyl)cyclopropyl]-2-[4-(ethylcarbamoylamino)phenyl]pyrimidin-4-yl]morpholine-3-carboxamide

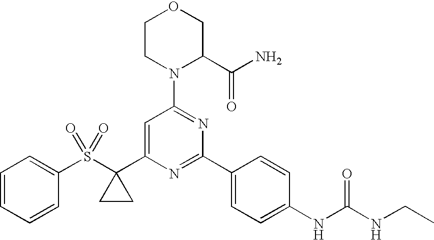

DIPEA (0.411 mL, 2.36 mmol) was added to [2-(4-aminophenyl)-6-[1-(benzenesulfonyl)cyclopropyl]pyrimidin-4-yl]trifluoromethanesulfonate (0.102 g, 0.20 mmol) and morpholine-3-carboxamide (as a mixture of the trifluoroacetic acid salts and the hydrochloride salt) (0.212 g, 1.63 mmol) in dioxane (5 mL) under nitrogen. The reaction was stirred at 70° C. for several hours. The solvent was removed and then the gum was taken up in dioxane again. Ethyl isocyanate (0.032 mL, 0.41 mmol) was added and the reaction stirred at RT over the weekend. Further ethyl isocyanate (an excess) was added and the reaction stirred for several days. Methanol was added and the solvent was removed. The crude product was purified by preparative HPLC, eluting with decreasingly polar mixtures of water (containing 1% ammonia) and acetonitrile, to give the desired material as a yellow solid (0.027 g).

NMR Spectrum: ¹H NMR (400 MHz, DMSO-d₆) δ 1.05-1.08 (3H, t), 1.59-1.62 (1H, m), 1.65-1.68 (1H, m), 1.87-1.95 (2H, m), 3.09-3.17 (2H, m), 3.17-3.19 (1H, d), 3.41-3.54 (3H, m), 3.70-3.73 (1H, dd), 3.94-3.97 (1H, d), 4.30-4.33 (1H, d), 6.13-6.16 (1H, t), 6.72 (1H, s), 7.15 (1H, bs), 7.36-7.38 (2H, d), 7.52 (1H, bs), 7.57-7.61 (2H, t), 7.70-7.74 (1H, tt), 7.79-7.82 (4H, m), 8.62 (1H, s).

LCMS Spectrum: m/z (ES+)(M+H)+=551; HPLC tR=1.84 min.

The preparation of [2-(4-aminophenyl)-6-[1-(benzenesulfonyl)cyclopropyl]pyrimidin-4-yl]trifluoromethanesulfonate was described earlier

### Morpholine-3-carboxamide

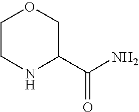

Hydrogen chloride (2.420 mL, 9.68 mmol) (4M solution in dioxane) was added to tert-butyl 3-cyanomorpholine-4-carboxylate (0.419 g, 1.98 mmol) in dioxane (15 mL) and the resulting solution stirred at RT over the weekend. The solvent was removed. The solid was taken up in dioxane and trifluoroacetic acid (1.2 eq) added. The reaction was allowed to stir at RT overnight. The solvent was removed, the crude material taken up in DCM and trifluoroacetic acid (0.734 mL, 9.88 mmol) added. The reaction was stirred overnight and then the solvent was removed to give the desired material (isolated as potentially a mixture of the trifluoroacetic acid salts and the hydrochloride salt). The material was used without further purification.

NMR Spectrum: ¹H NMR (400 MHz, DMSO-d₆) δ 3.07-3.16 (1H, m), 3.58-3.70 (1H, m), 3.79-3.82 (1H, m), 3.87-3.93 (1H, m), 4.00-4.07 (1H, m), 4.11-4.22 (1H, m), 4.71-4.89 (1H, m), 7.76-8.16 (t) (TFA salt), 9.15-9.78 (bt) (HCl salt). tert-Butyl 3-cyanomorpholine-4-carboxylate

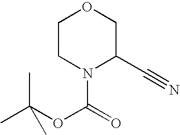

tert-Butyl 3-carbamoylmorpholine-4-carboxylate (0.929 g, 4.03 mmol) was dissolved in dry DCM (10 mL) and triethylamine (1.181 mL, 8.47 mmol) was added. The solution was put under nitrogen and cooled to 0° C. Trifluoroacetic anhydride (0.627 mL, 4.44 mmol) was then added and the reaction was allowed to slowly warm up to RT, followed by stirring at RT for at least 3 hours. The solvent was removed and then ethyl acetate added. The organic layer was washed with brined, dried over MgSO₄, filtered and evaporated. The crude product was purified by flash silica chromatography, elution gradient 0 to 30% ethyl acetate in DCM, to give the desired material as a light yellow crystalline solid (0.419 g).

NMR Spectrum: ¹H NMR (400 MHz, CDCl₃) δ 1.50 (9H, s), 3.24 (1H, bt), 3.45-3.52 (1H, td), 3.60-3.64 (1H, dd), 3.80-3.83 (1H, d), 3.94-3.97 (1H, d), 4.04-4.07 (1H, d), 4.89 (1H, bs). tert-butyl 3-carbamoylmorpholine-4-carboxylate

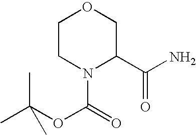

HATU (5.92 g, 15.57 mmol) was added to a solution of 4-(tert-butoxycarbonyl)morpholine-3-carboxylic acid (3 g, 12.97 mmol), DIPEA (3.40 mL, 19.46 mmol) and ammonium chloride (3.47 g, 64.87 mmol) in DMF (70 mL) and the resulting suspension stirred at RT for 12 hours under nitrogen. The reaction mixture was concentrated and diluted with ethyl acetate (150 mL), and washed sequentially with water (2×50 mL), and saturated brine (50 mL). The organic layer was dried over MgSO₄, filtered and evaporated to afford the desired material as an oil (4.0 g), which was used without further purification.

NMR Spectrum: ¹H NMR (400 MHz, DMSO-d₆) δ 1.39 (9H, s), 3.15 (2H, m), 3.54 (2H, m), 3.63 (2H, m), 3.75 (1H, m), 4.17 (2H, m).

### EXAMPLE 90

### 4-[6-[1-(Benzenesulfonyl)cyclopropyl]-2-[4-(ethylcarbamoylamino)phenyl]pyrimidin-4-yl]-N,N-dimethylmorpholine-3-carboxamide

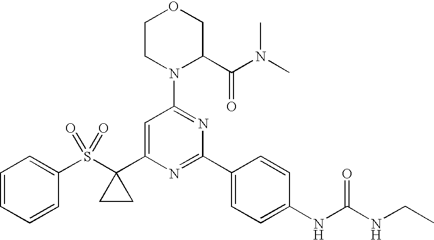

DIPEA (0.141 mL, 0.81 mmol) was added to [2-(4-aminophenyl)-6-[1-(benzenesulfonyl)cyclopropyl]pyrimidin-4-yl]trifluoromethanesulfonate (0.101 g, 0.20 mmol) and N,N-dimethylmorpholine-3-carboxamide (as the hydrochloride salt) (0.079 g, 0.41 mmol) in dioxane (5 mL) under nitrogen. The reaction was stirred at 70° C. for several hours then allowed to cool and ethyl isocyanate (0.321 mL, 4.05 mmol) added. The reaction was stirred at RT overnight, additional ethyl isocyanate added and the reaction stirred at RT overnight. Methanol was added and the solvent was removed. The crude product was purified by preparative HPLC, eluting with decreasingly polar mixtures of water (containing 1% ammonia) and acetonitrile, to give the desired material as a cream solid (0.047 g).

NMR Spectrum: ¹H NMR (400 MHz, DMSO-d₆) δ 1.05-1.08 (3H, t), 1.58-1.62 (1H, m), 1.68-1.73 (1H, m), 1.86-1.97 (2H, m), 2.82 (3H, bs), 3.09-3.16 (2H, m), 3.19 (3H, bs), 3.53-3.60 (1H, m), 3.67-3.71 (1H, m), 3.79-3.84 (1H, dd), 4.00-4.02 (1H, d), 4.14-4.17 (1H, d), 5.44 (1H, bs), 6.15-6.18 (1H, t), 6.77 (1H, s), 7.37-7.39 (2H, d), 7.56-7.60 (2H, t), 7.70-7.74 (1H, t), 7.76-7.80 (4H, m), 8.61 (1H, s). (1 peak under water or solvent peak).

LCMS Spectrum: m/z (ES+)(M+H)+=579; HPLC tR=2.01 min.

The preparation of [2-(4-aminophenyl)-6-[1-(benzenesulfonyl)cyclopropyl]pyrimidin-4-yl]trifluoromethanesulfonate was described earlier

### N,N-Dimethylmorpholine-3-carboxamide

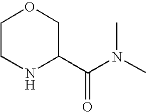

Hydrogen chloride (3.57 mL, 14.28 mmol) (4M solution in dioxane) was added to tert-butyl 3-(dimethylcarbamoyl)morpholine-4-carboxylate (0.820 g, 3.17 mmol) in dioxane (25 mL) and the resulting solution stirred at RT overnight. The solvent was removed and the gum was triturated with diethyl ether to give the desired material (as the hydrochloride salt) as a solid which turned to a gum on standing (0.70 g).

NMR Spectrum: ¹H NMR (400 MHz, CDCl₃) δ 2.89 (3H, s), 3.07 (3H, s), 3.13-3.16 (1H, dd), 3.20-3.23 (1H, dt), 3.42-3.48 (1H, m), 3.69-3.75 (1H, td), 3.92-3.96 (1H, dt), 4.15-4.19 (1H, dd), 4.57-4.61 (1H, dd), 9.47 (1H, bs).

### tert-Butyl 3-(dimethylcarbamoyl)morpholine-4-carboxylate

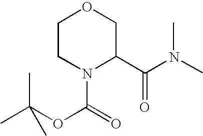

HATU (1.97 g, 5.19 mmol) was added to a solution of 4-(tert-butoxycarbonyl)morpholine-3-carboxylic acid (1 g, 4.32 mmol), DIPEA (1.133 ml, 6.49 mmol) and dimethylamine 2.0M in THF (10.81 mL, 21.62 mmol) in DMF and the resulting solution stirred at RT for 15 hours under nitrogen. The reaction mixture was concentrated and diluted with ethyl acetate (100 mL), and washed sequentially with water(25 mL), 5% aqueous citric acid solution (25 mL), and saturated brine (25 mL). The organic layer was dried over MgSO₄, filtered and evaporated to afford crude product and dried under vacuum to the desired material as an oil (0.91 g), which was used without further purification.

NMR Spectrum: ¹H NMR (400 MHz, DMSO-d₆) δ 1.36 (9H, br s), 2.83 (3H, s), 2.98 (3H, s), 3.35 (1H, m), 3.48 (1H, m), 3.63 (1H, dd), 3.80 (1H, m), 3.90-4.04 (2H, m), 4.70 (1H, m).

### EXAMPLE 91

### 3-Cyclopropyl-1-[4-[4-[(3S,5S)-3,5-dimethylmorpholin-4-yl]-6-(1-methylsulfonylyclopropyl)pyrimidin-2-yl]phenyl]urea

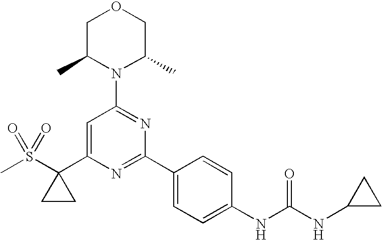

Cyclopropylamine (0.10 mmol) was added to phenyl N-[4-[4-[(3S,5S)-3,5-dimethylmorpholin-4-yl]-6-(1-methylsulfonylcyclopropyl)pyrimidin-2-yl]phenyl]carbamate (50 mg, 0.10 mmol) and triethylamine (0.027 mL, 0.19 mmol) in DMF (1 mL) at RT. The resulting solution was stirred at 50° C. for 2 hours and the material purified by preparative HPLC, eluting with decreasingly polar mixtures of water (containing 1% ammonia) and acetonitrile, to give the desired material as a white solid (40 mg).

NMR Spectrum: ¹H NMR (400 MHz, DMSO-d₆) δ 0.40-0.44 (2H, m), 0.63-0.67 (2H, m), 1.36 (6H, d), 1.53-1.61 (2H, m), 1.64-1.72 (2H, m), 2.54-2.59 (1H, m), 3.26 (3H, s), 3.72 (2H, dd), 4.16 (2H, dd), 4.25-4.31 (2H, m), 6.44 (1H, d), 6.72 (1H, s), 7.52 (2H, d), 8.21 (2H, d), 8.54 (1H, s).

LCMS Spectrum: m/z (ES+)(M+H)+=486; HPLC tR=2.13 min.

The compounds below were prepared in an analogous fashion from phenyl N-[4-[4-[(3S,5S)-3,5-dimethylmorpholin-4-yl]-6-(1-methylsulfonylcyclopropyl)pyrimidin-2-yl]phenyl]carbamate and the appropriate amine.

| | | | LCMS | Retention |
|---|---|---|---|---|
| Example | Structure | NAME | MH+ | time (min) |
| 91a | 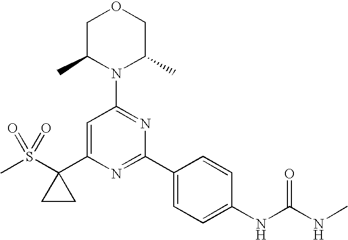 | 1-[4-[4-[(3S,5S)-3,5-dimethylmorpholin-4-yl]-6-(1-methylsulfonylcyclopropyl)pyrimidin-2-yl]phenyl]-3-methylurea | 460 | 1.70 |
| 91b | 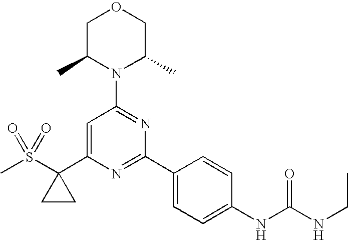 | 1-[4-[4-[(3S,5S)-3,5-dimethylmorpholin-4-yl]-6-(1-methylsulfonylcyclopropyl)pyrimidin-2-yl]phenyl]-3-ethylurea | 474 | 2.11 |
| 91c | 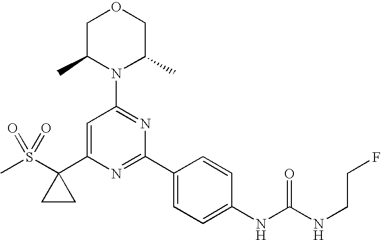 | 1-[4-[4-[(3S,5S)-3,5-dimethylmorpholin-4-yl]-6-(1-methylsulfonylcyclopropyl)pyrimidin-2-yl]phenyl]-3-(2-fluoroethyl)urea | 492 | 2.11 |
| 91d | 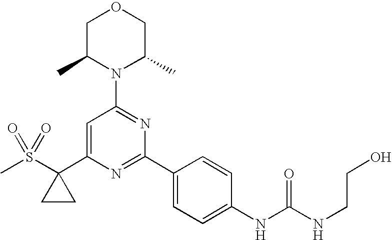 | 1-[4-[4-[(3S,5S)-3,5-dimethylmorpholin-4-yl]-6-(1-methylsulfonylcyclopropyl)pyrimidin-2-yl]phenyl]-3-(2-hydroxyethyl)urea | 490 | 1.77 |

### EXAMPLE 91a

¹H NMR (400.132 MHz, DMSO-d₆) δ 1.36 (6H, d), 1.55-1.61 (2H, m), 1.64-1.70 (2H, m), 2.66 (3H, d), 3.26 (3H, s), 3.72 (2H, dd), 4.16 (2H, dd), 4.27-4.28 (2H, m), 6.07-6.08 (1H, m), 6.72 (1H, s), 7.51 (2H, d), 8.20 (2H, d), 8.74 (1H, s).

### EXAMPLE 91b

¹H NMR (400.132 MHz, DMSO-d₆) δ 1.07 (3H, t), 1.36 (6H, d), 1.55-1.61 (2H, m), 1.64-1.73 (2H, m), 3.09-3.16 (2H, m), 3.26 (3H, s), 3.72 (2H, dd), 4.16 (2H, dd), 4.27-4.28 (2H, m), 6.17 (1H, t), 6.72 (1H, s), 7.50 (2H, d), 8.20 (2H, d), 8.66 (1H, s).

### EXAMPLE 91c

¹H NMR (400.132 MHz, DMSO-d₆) δ 1.36 (6H, d), 1.55-1.59 (2H, m), 1.64-1.70 (2H, m), 3.26 (3H, s), 3.42 (2H, dq), 3.72 (2H, dd), 4.16 (2H, dd), 4.26-4.29 (2H, m), 4.42 (1H, t), 4.54 (1H, t), 6.44 (1H, t), 6.72 (1H, s), 7.51 (2H, d), 8.21 (2H, d), 8.81 (1H, s).

### EXAMPLE 91d

¹H NMR (400.132 MHz, DMSO-d₆) δ 1.36 (6H, d), 1.55-1.60 (2H, m), 1.65-1.72 (2H, m), 3.16-3.20 (2H, m), 3.26 (3H, s), 3.44-3.48 (2H, m), 3.72 (2H, dd), 4.16 (2H, dd), 4.24-4.31 (2H, m), 4.73 (1H, t), 6.26 (1H, t), 6.72 (1H, s), 7.50 (2H, d), 8.20 (2H, d), 8.81 (1H, s).

The preparation of phenyl N-[4-[4-[(3S,5S)-3,5-dimethylmorpholin-4-yl]-6-(1-methylsulfonylcyclopropyl)pyrimidin-2-yl]phenyl]carbamate is described below.

### Phenyl N-[4-[4-[(3S,5S)-3 5-dimethylmorpholin-4-yl]-6-(1-methylsulfonylcyclopropyl)pyrimidin-2-yl]phenyl]carbamate

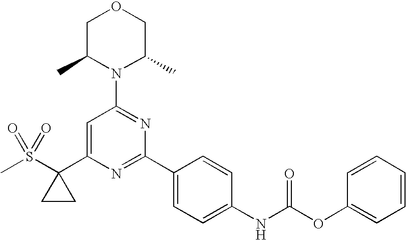

Sodium bicarbonate (104 mg, 1.24 mmol) was added to 4-[4-[(3S,5S)-3,5-dimethylmorpholin-4-yl]-6-(1-methylsulfonylcyclopropyl)pyrimidin-2-yl]aniline (250 mg, 0.62 mmol) in 1,4-dioxane (3 mL) and phenyl chloroformate (0.078 mL, 0.62 mmol) was added dropwise to the resulting suspension. The reaction stirred at RT for 2 hours then evaporated to dryness, redissolved in DCM (20 mL), and washed with water (20 mL). The organic layer was dried over MgSO₄, filtered and evaporated to afford a solid which was triturated with diethyl ether to give the desired material as a cream solid (250 mg).

NMR Spectrum: ¹H NMR (400 MHz, DMSO-d₆) δ 1.37 (6H, d), 1.56-1.62 (2H, m), 1.65-1.71 (2H, m), 3.27 (3H, s), 3.73 (2H, dd), 4.17 (2H, dd), 4.29-4.31 (2H, m), 6.77 (1H, s), 7.24-7.30 (3H, m), 7.45 (2H, t), 7.65 (2H, d), 8.30 (2H, d), 10.45 (1H, s).

LCMS Spectrum: m/z (ES+)(M+H)+=523; HPLC tR=2.74 min.

### 4-[4-[(3S,5S)-3,5-Dimethylmorpholin-4-yl]-6-(1-methylsulfonylcyclopropyl)pyrimidin-2--yl]aniline

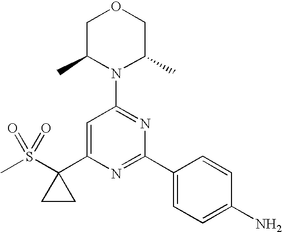

Bis(triphenylphosphine)palladium(II) chloride (35.5 mg, 0.05 mmol) was added to 2-chloro-4-[(3S,5S)-3,5-dimethylmorpholin-4-yl]-6-(1-methylsulfonylcyclopropyl)pyrimidine (350 mg, 1.01 mmol), 4-(4,4,5,5-tetramethyl-1,3,2-dioxaborolan-2-yl)aniline (222 mg, 1.01 mmol) and 2M aqueous sodium carbonate solution (1.52 mL, 3.04 mmol), in ethanol (0.89 mL), DME (1.77 mL) and water (0.89 mL) and the reaction stirred at 90° C. for 2 hours. The reaction mixture was diluted with ethyl acetate (20 mL), and washed sequentially with water (20 mL), and saturated brine (20 mL). The organic layer was dried over MgSO₄, filtered and evaporated to afford crude product. The crude product was purified by flash silica chromatography, elution gradient 2 to 10% methanol in DCM, to give the desired material as a beige solid (250 mg).

NMR Spectrum: ¹H NMR (400 MHz, DMSO-d₆) δ 1.34 (6H, d), 1.50-1.58 (2H, m), 1.62-1.70 (2H, m), 3.25 (3H, s), 3.70 (2H, dd), 4.14 (2H, dd), 4.22-4.25 (2H, m), 5.56 (1H, s), 6.61 (2H, d), 8.04 (2H, d).

LCMS Spectrum: m/z (ES+)(M+H)+=403; HPLC tR=2.05 min.

### 2-Chloro-4-[(3S,5S)-3,5-dimethylmorpholin-4-yl]-6-(1-methylsulfonylcyclopropyl)pyrimidine

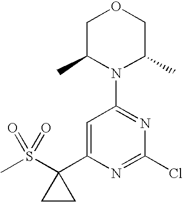

A solution of 50% w/v sodium hydroxide (6.12 mL, 154.44 mmol) was added portionwise to a stirred solution of 2-chloro-4-[(3S,5S)-3,5-dimethylmorpholin-4-yl]-6-(methylsulfonylmethyl)pyrimidine (898 mg, 2.81 mmol), tetrabutylammonium bromide (91 mg, 0.28 mmol) and 1,2-dibromoethane (0.726 mL, 8.42 mmol) in toluene (50 mL) and the resulting suspension stirred at 60° C. for 6 hours. The reaction mixture was diluted with water (50 mL), and washed sequentially with water (2×50 mL), and saturated brine (50 mL). The organic layer was dried over MgSO₄, filtered and evaporated to afford crude product. The crude product was purified by flash silica chromatography, elution gradient 0 to 20% ethyl acetate in DCM, to give the desired material as a solid (350 mg).

NMR Spectrum: ¹H NMR (400 MHz, CDCl₃) δ 1.43 (3H, d), 1.44 (3H, d), 1.50 (2H, m), 1.82 (2H, m), 3.02 (3H, s), 3.78 (2H, dd), 4.16 (2H, m), 4.24 (2H, dd), 6.77 (1H, s).

LCMS Spectrum: m/z (ES+)(M+H)+=346; HPLC tR=1.83 min.

### 2-Chloro-4-[(3S,5S)-3,5-dimethylmorpholin-4-yl]-6-(methylsulfonylmethyl)pyrimidine

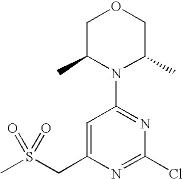

Sodium methanesulfinate (0.542 g, 5.31 mmol) was added portionwise to 2-chloro-4-[(3S,5S)-3,5-dimethylmorpholin-4-yl]-6-(iodomethyl)pyrimidine (1.86 g, 5.06 mmol) in acetonitrile (50 mL) and the resulting suspension stirred at 80° C. for 6 hours. Further sodium methanesulfinate (0.5 equivalents) was added and the reaction was heated a further 8 hours. The solvent was removed and the solid was taken up in DMF. Further sodium methanesulfinate (1 equivalent) was added and the mixture allowed to stir until the reaction was complete. The solvent was removed and ethyl acetate added. The mixture was washed with 10% sodium thiosulfate, brine and water and the organics dried over MgSO₄, filtered and evaporated. The crude product was purified by flash silica chromatography, elution gradient 0 to 25% ethyl acetate in DCM, to give the desired material as a yellow gum (0.898 g).

NMR Spectrum: ¹H NMR (400 MHz, CDCl₃) δ 1.44-1.45 (6H, d), 3.02 (3H, s), 3.77-3.80 (2H, dd), 4.14-4.16 (2H, m), 4.17-4.18 (2H, d), 4.23-4.27 (2H, dd), 6.46 (1H, s).

LCMS Spectrum: m/z (ES+)(M+H)+=320; HPLC tR=1.59 min.

### 2-Chloro-4-[(3S,5S)-3,5-dimethylmorpholin-4-yl]-6-(iodomethyl)pyrimidine

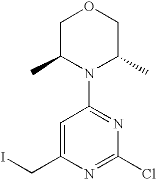

[2-Chloro-6-[(3S,5S)-3,5-dimethylmorpholin-4-yl]pyrimidin-4-yl]methyl methanesulfonate (5.21 g, 15.51 mmol) and lithium iodide (1.190 mL, 31.03 mmol) were added to dioxane (250 mL) and heated at 60° C. for 1 hour and then at RT overnight. The mixture was evaporated to dryness and partitioned between saturated ammonium chloride solution (100 mL) and DCM (75 mL). The layers were separated and the aqueous layer further extracted with DCM (2×75 mL) then the combined organics washed sequentially with 10% sodium thiosulfate solution (100 mL) and saturated brine (50 mL). The organic layer was dried over MgSO₄, filtered and evaporated to afford crude product as a brown oil (5.59 g). The material was used without further purification.

NMR Spectrum: ¹H NMR (400 MHz, DMSO-d₆) δ 1.32 (6H, d), 3.69 (2H, dd), 4.08 (2H, m), 4.14 (2H, dd), 4.34 (2H, s), 6.84 (1H, s).

LCMS Spectrum: m/z (ES+)(M+H)+=368; HPLC tR=2.26 min.

### [2-Chloro-6-[(3S,5S)-3,5-dimethylmorpholin-4-yl]pyrimidin-4-yl]methyl methanesulfonate

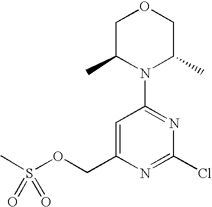

Methanesulfonyl chloride (1.802 mL, 23.28 mmol) was added dropwise to [2-chloro-6-[(3S,5S)-3,5-dimethylmorpholin-4-yl]pyrimidin-4-yl]methanol (4.00 g, 15.52 mmol) and DIPEA (4.03 mL, 23.28 mmol) in DCM (100 mL) at 0° C. over a period of 2 minutes and the resulting solution allowed to gradually warm up to RT over a period of 2 hours. The reaction mixture was diluted with DCM (50 mL), and washed with water. The organic layer was dried over MgSO₄, filtered and evaporated to afford the desired material as a yellow gum (5.56 g). This was used without further purification.

NMR Spectrum: ¹H NMR (400 MHz, CDCl₃) δ 1.42-1.44 (6H, d), 3.14 (3H, s), 3.76-3.80 (2H, dd), 4.13-4.18 (2H, m), 4.22-4.26 (2H, dd), 5.11 (2H, d), 6.48 (1H, s).

LCMS Spectrum: m/z (ES+)(M+H)+=336; HPLC tR=1.88 min.

### [2-Chloro-6-[(3S,5S)-3 5-dimethylmorpholin-4-yl]pyrimidin-4-yl]methanol

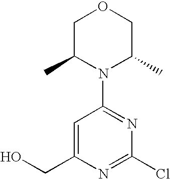

Lithium borohydride, 2M in THF (6.54 mL, 13.09 mmol) was added dropwise to methyl 2-chloro-6-[(3S,5S)-3,5-dimethylmorpholin-4-yl]pyrimidine-4-carboxylate (4.40 g, 15.40 mmol) in THF (75 mL) at 0° C. over a period of 30 minutes under nitrogen. The resulting solution was stirred at 0° C. for 30 minutes then allowed to warm to RT. Water (250 mL) was added and the organics removed in vacuo. The aqueous residues were extracted with ethyl acetate then the combined organics were washed with brine. The organic layer was dried over MgSO₄ then evaporated to dryness to afford the desired material as a white solid (4.0 g).

NMR Spectrum: ¹H NMR (400 MHz, CDCl₃) δ 1.41-1.42 (6H, d), 2.69-2.71 (1H, t), 3.75-3.78 (2H, dd), 4.12-4.18 (2H, m), 4.21-4.25 (2H, dd), 4.59-4.60 (2H, d), 6.39 (1H, s).

LCMS Spectrum: m/z (ES+)(M+H)+=258; HPLC tR=1.38 min.

### Methyl 2-chloro-6-[(3S,5S)-3,5-dimethylmorpholin-4-yl]pyrimidine-4-carboxylate

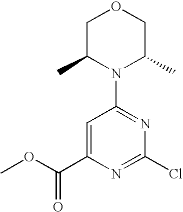

Methyl 2,4-dichloropyrimidine-6-carboxylate (4.45 g, 21.50 mmol) was dissolved in dry DCM (100 mL) and DIPEA (9.67 mL, 55.89 mmol) was added. (3S,5S)-3,5-Dimethylmorpholine (as the hydrochloride salt) (3.42 g, 22.57 mmol)in DCM (20 mL) was added to this solution dropwise over several minutes and the reaction allowed to stir at RT for days then at 50° C. for several days. The crude reaction mixture was washed with water, dried over MgSO₄ and filtered. The crude product was purified by flash silica chromatography, elution gradient 0 to 25% ethyl acetate in DCM, to give the desired material as a cream solid (4.4 g).

NMR Spectrum: ¹H NMR (400 MHz, CDCl₃) δ 1.44-1.46 (6H, d), 3.78-3.81 (2H, dd), 3.98 (3H, s), 4.16-4.22 (2H, m), 4.24-4.28 (2H, dd), 7.10 (1H, s).

LCMS Spectrum: m/z (ES+)(M+H)+=286; HPLC tR=1.72 min.

The preparation of (3S,5S)-3,5-dimethylmorpholine was described earlier.

### EXAMPLE 92

### 3-Cyclopropyl-1-[4-[4-(1-cyclopropylsulfonylcyclopropyl)-6-[(3S,5S)-3,5-dimethylmorpholin-4-yl]pyrimidin-2-yl]phenyl]urea

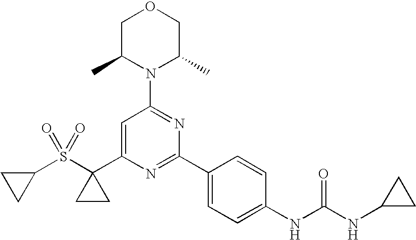

Cyclopropanamine (12.49 mg, 0.22 mmol) was added to phenyl N-[4-[4-(1-cyclopropylsulfonylcyclopropyl)-6-[(3S,5S)-3,5-dimethylmorpholin-4-yl]pyrimidin-2-yl]phenyl]carbamate (120 mg, 0.22 mmol) and triethylamine (0.091 mL, 0.66 mmol) in DMF (1 mL) at RT. The resulting solution was stirred at 50° C. for 2 hours. The crude product was purified by preparative HPLC, eluting with decreasingly polar mixtures of water (containing 1% ammonia) and acetonitrile, to give the desired material as a white solid (82 mg).

NMR Spectrum: ¹H NMR (400 MHz, DMSO-d₆) δ 0.40-0.44 (2H, m), 0.63-0.67 (2H, m), 0.87-1.04 (4H, m), 1.36 (6H, d), 1.57-1.70 (4H, m), 2.54-2.58 (1H, m), 2.91-2.97 (1H, m), 3.71 (2H, dd), 4.16 (2H, dd), 4.25-4.27 (2H, m), 6.43 (1H, s), 6.81 (1H, s), 7.51 (2H, d), 8.22 (2H, d), 8.52 (1H, s).

LCMS Spectrum: m/z (ES+)(M+H)+=512; HPLC tR=2.25 min.

The compounds below were prepared in an analogous fashion from phenyl N-[4-[4-(1-cyclopropylsulfonylcyclopropyl)-6-[(3S,5S)-3,5-dimethylmorpholin-4-yl]pyrimidin-2-yl]phenyl]carbamate and the appropriate amine.

| | | | | Retention |
|---|---|---|---|---|
| | | | LCMS | time |
| Example | Structure | NAME | MH+ | (min) |
| 92a | 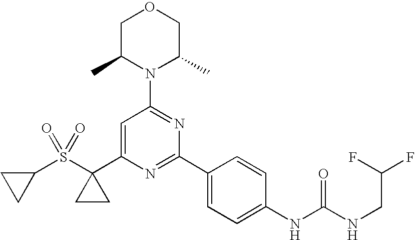 | 1-[4-[4-(1-cyclopropylsulfonylcyclopropyl)-6-[(3S,5S)-3,5-dimethylmorpholin-4-yl]pyrimidin-2-yl]phenyl]-3-(2,2-difluoroethyl)urea | 537 | 2.39 |
| 92b | 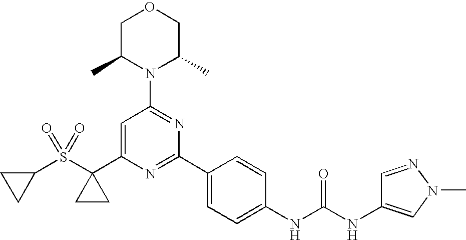 | 1-[4-[4-(1-cyclopropylsulfonylcyclopropyl)-6-[(3S,5S)-3,5-dimethylmorpholin-4-yl]pyrimidin-2-yl]phenyl]-3-(1-methylpyrazol-4-yl)urea | 552 | 1.83 |
| 92c | 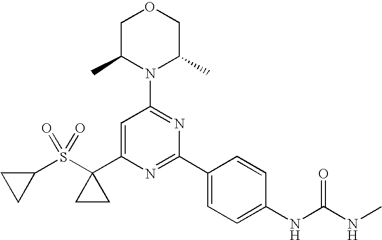 | 1-[4-[4-(1-cyclopropylsulfonylcyclopropyl)-6-[(3S,5)-3,5-dimethylmorpholin-4-yl]pyrimidin-2-yl]phenyl]-3-methylurea | 486 | 2.06 |
| 92d | 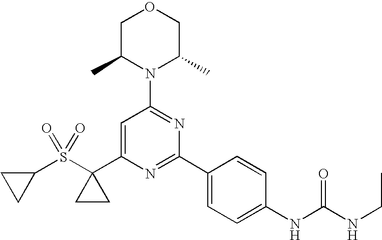 | 1-[4-[4-(1-cyclopropylsulfonylcyclopropyl)-6-[(3S,5S)-3,5-dimethylmorpholin-4-yl]pyrimidin-2-yl]phenyl]-3-ethylurea | 500 | 1.91 |
| 92e | 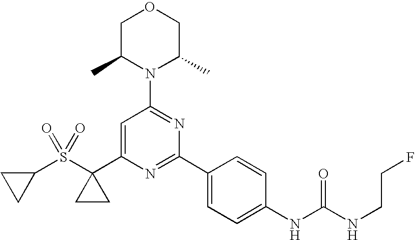 | 1-[4-[4-(1-cyclopropylsulfonylcyclopropyl)-6-[(3S,5S)-3,5-dimethylmorpholin-4-yl]pyrimidin-2-yl]phenyl]-3-(2-fluoroethyl)urea | 518 | 1.94 |
| 92f | 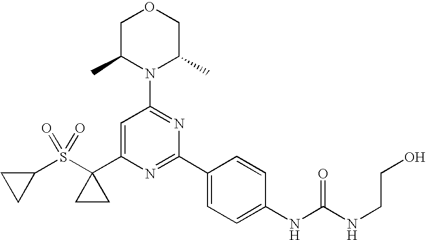 | 1-[4-[4-(1-cyclopropylsulfonylcyclopropyl)-6-[(3S,5S)-3,5-dimethylmorpholin-4-yl]pyrimidin-2-yl]phenyl]-3-(2-hydroxyethyl)urea | 516 | 1.88 |

### EXAMPLE 92a

¹H NMR (400.132 MHz, DMSO-d₆) δ 0.87-1.06 (4H, m), 1.36 (6H, d), 1.55-1.70 (4H, m), 2.91-2.97 (1H, m), 3.50-3.60 (2H, m), 3.72 (2H, dd), 4.16 (2H, dd), 4.25-4.28 (2H, m), 6.07 (1H, t), 6.54 (1H, t), 6.82 (1H, s), 7.52 (2H, d), 8.24 (2H, d), 8.91 (1H, s).

### EXAMPLE 92b

¹H NMR (400.132 MHz, DMSO-d₆) δ 0.88-1.07 (4H, m), 1.36 (6H, d), 1.56-1.71 (4H, m), 2.91-2.98 (1H, m), 3.72 (2H, dd), 3.79 (3H, s), 4.16 (2H, dd), 4.26-4.29 (2H, m), 6.82 (1H, s), 7.39 (1H, s), 7.55 (2H, d), 7.76 (1H, s), 8.25 (2H, d), 8.39 (1H, s), 8.83 (1H, s).

### EXAMPLE 92c

¹H NMR (400.132 MHz, DMSO-d₆) δ 0.89-1.04 (4H, m), 1.36 (6H, d), 1.57-1.68 (4H, m), 2.66 (3H, d), 2.91-2.97 (1H, m), 3.71 (2H, ddd), 4.16 (2H, dd), 4.25-4.27 (2H, m), 6.06-6.07 (1H, m), 7.50 (2H, d), 7.55-7.58 (1H, m), 8.21 (2H, d), 8.73 (1H, s).

### EXAMPLE 92d

¹H NMR (400.132 MHz, DMSO-d₆) δ 0.87-1.04 (4H, m), 1.07 (3H, t), 1.36 (6H, d), 1.55-1.70 (4H, m), 2.91-2.97 (1H, m), 3.09-3.16 (2H, m), 3.71 (2H, dd), 4.16 (2H, dd), 4.24-4.27 (2H, m), 6.16 (1H, t), 6.81 (1H, s), 7.49 (2H, d), 8.21 (2H, d), 8.65 (1H, s).

### EXAMPLE 92e

¹H NMR (400.132 MHz, DMSO-d₆) δ 0.87-1.06 (4H, m), 1.36 (6H, d), 1.55-1.70 (4H, m), 2.91-2.97 (1H, m), 3.39 (1H, q), 3.46 (1H, q), 3.72 (2H, dd), 4.16 (2H, dd), 4.22-4.29 (2H, m), 4.42 (1H, t), 4.54 (1H, t), 6.44 (1H, t), 6.81 (1H, s), 7.51 (2H, d), 8.23 (2H, d), 8.80 (1H, s).

### EXAMPLE 92f

¹H NMR (400.132 MHz, DMSO-d₆) δ 0.87-1.04 (4H, m), 1.36 (6H, d), 1.55-1.69 (4H, m), 2.90-2.97 (1H, m), 3.16-3.20 (2H, m), 3.44-3.48 (2H, m), 3.71 (2H, dd), 4.16 (2H, dd), 4.24-4.29 (2H, m), 4.73 (1H, t), 6.26 (1H, t), 6.81 (1H, s), 7.49 (2H, d), 8.22 (2H, d), 8.79 (1H, s).

The preparation of phenyl N-[4-[4-(1-cyclopropylsulfonylcyclopropyl)-6-[(3S,5S)-3,5-dimethylmorpholin-4-yl]pyrimidin-2-yl]phenyl]carbamate is described below.

### Phenyl N-[4-[4-(1-cyclopropylsulfonylcyclopropyl)-6-[(3S,5s)-3,5-dimethylmorpholin-4-yl]pyrimidin-2-yl]phenyl]carbamate

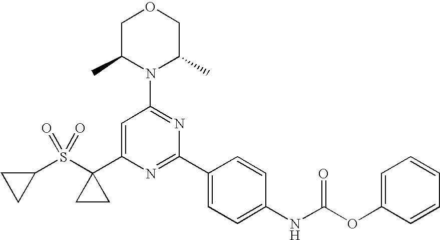

Sodium bicarbonate (286 mg, 3.41 mmol) was added to 4-[4-(1-cyclopropylsulfonylcyclopropyl)-6-[(3S,5S)-3,5-dimethylmorpholin-4-yl]pyrimidin-2-yl]aniline (730 mg, 1.70 mmol), in 1,4-dioxane (8.3 mL) and phenyl chloroformate (0.214 mL, 1.70 mmol) added dropwise to the resulting suspension. The mixture was stirred at RT for 2 hours. The reaction mixture was evaporated to dryness and redissolved in DCM (20 mL), and washed with water (20 mL). The organic layer was dried over MgSO₄, filtered and evaporated to afford a solid which was triturated with diethyl ether to give the desired material as a cream solid (800 mg).

NMR Spectrum: ¹H NMR (400 MHz, DMSO-d₆) δ 0.88-1.05 (4H, m), 1.37 (6H, d), 1.59-1.70 (4H, m), 2.92-2.99 (1H, m), 3.73 (2H, dd), 4.17 (2H, dd), 4.28-4.30 (2H, m), 6.86 (1H, s), 7.24-7.30 (3H, m), 7.45 (2H, t), 7.64 (2H, d), 8.30 (2H, d), 10.45 (1H, s).

LCMS Spectrum: m/z (ES+)(M+H)+=549; HPLC tR=2.66 min.

### 4-[4-(1-Cyclopropylsulfonylcyclopropyl)-6-[(3S,5S)-3,5-dimethylmorpholin-4-yl]pyrimidin-2-yl]aniline

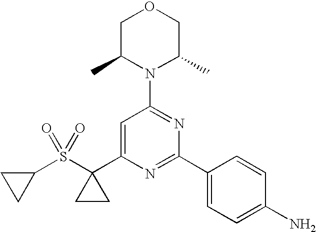

Bis(triphenylphosphine)palladium(II) chloride (60.4 mg, 0.09 mmol) was added to 2-chloro-4-(1-cyclopropylsulfonylcyclopropyl)-6-[(3S,5S)-3,5-dimethylmorpholin-4-yl]pyrimidine (640 mg, 1.72 mmol), 4-(4,4,5,5-tetramethyl-1,3,2-dioxaborolan-2-yl)aniline (490 mg, 2.24 mmol) and 2M sodium carbonate solution (2.6 mL, 5.16 mmol) in water (1.5 mL), ethanol (1.5 mL), and DME (3 mL) and the resulting solution was stirred at 95° C. for 2 hours. The reaction mixture was diluted with ethyl acetate (20 mL), and washed sequentially with water (10 mL) and saturated brine (10 mL). The organic layer was dried over MgSO₄, filtered and evaporated to afford crude product. The crude product was purified by flash silica chromatography, elution gradient 0 to 10% methanol in DCM, to give the desired material as a beige solid (740 mg).

NMR Spectrum: ¹H NMR (400 MHz, DMSO-d₆) δ 0.89-1.03 (4H, m), 1.34 (6H, d), 1.54-1.67 (4H, m), 2.89-2.96 (1H, m), 3.70 (2H, dd), 4.14 (2H, dd), 4.20-4.24 (2H, m), 6.61 (2H, d), 6.73 (1H, s), 8.05 (2H, d)

LCMS Spectrum: m/z (ES+)(M+H)+=429; HPLC tR=2.18 min.

### 2-Chloro-4-(1-cyclopropylsulfonylcyclopropyl)-6-[(3S,5S)-3 5-dimethylmorpholin-4-yl]pyrimidine

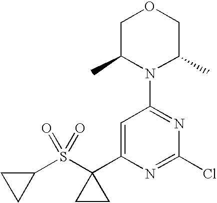

A solution of 50% w/v sodium hydroxide (7.06 mL, 176.52 mmol) was added portionwise to a stirred solution of 2-chloro-4-(cyclopropylsulfonylmethyl)-6-[(3S,5S)-3,5-dimethylmorpholin-4-yl]pyrimidine (1.11 g, 3.21 mmol), tetrabutylammonium bromide (0.103 g, 0.32 mmol) and 1,2-dibromoethane (0.830 mL, 9.63 mmol) in toluene (50 mL) and the resulting suspension stirred at 60° C. for 6 hours. The reaction mixture was diluted with water (50 mL), and washed sequentially with water (2×50 mL), and saturated brine (50 mL). The organic layer was dried over MgSO₄, filtered and evaporated to afford crude product. The crude product was purified by flash silica chromatography, elution gradient 0 to 20% ethyl acetate in DCM, to give the desired material as a solid (0.64 g).

NMR Spectrum: ¹H NMR (400 MHz, DMSO-d₆) δ 0.86-0.99 (2H, m), 1.01 (2H, m), 1.31 (3H, d), 1.33 (3H, d), 1.51 (2H, m), 1.63 (2H, m), 2.86 (1H, m), 3.70 (2H, dd), 4.13 (2H, m), 4.16 (2H, m), 6.90 (1H, s).

LCMS Spectrum: m/z (ES+)(M+H)+=372; HPLC tR=1.97 min.

### 2-Chloro-4-(cyclopropylsulfonylmethyl)-6-[(3S,5S)-3,5-dimethylmorpholin-4-yl]pyrimidine

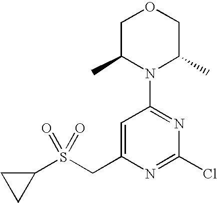

Sodium cyclopropanesulfinate (0.648 g, 5.06 mmol) was added portionwise to 2-chloro-4-[(3S,5S)-3,5-dimethylmorpholin-4-yl]-6-(iodomethyl)pyrimidine (1.86 g, 5.06 mmol) in acetonitrile (50 mL) and the resulting suspension stirred at 80° C. for 6 hours. Further sodium cyclopropanesulfinate (259 mg, 2.02 mmol) was added in one portion and the suspension was stirred at 80° C. for a further 2 hours. The reaction mixture was concentrated and diluted with DCM (200 mL), and washed sequentially with water (50 mL), 10% sodium thiosulfate solution (50 mL), and saturated brine(50 mL). The organic layer was dried over MgSO₄, filtered and evaporated to afford crude product. The crude product was purified by flash silica chromatography, elution gradient 0 to 20% ethyl acetate in DCM, to give the desired material as a yellow solid (1.11 g).

NMR Spectrum: ¹H NMR (400 MHz, DMSO-d₆) δ 0.97 (2H, m), 1.03 (2H, m), 1.32 (3H, d), 1.34 (3H, d), 2.79 (1H, m), 3.70 (2H, dd), 4.09 (2H, m), 4.15 (2H, dd), 4.49 (2H, d), 6.82 (1H, s).

LCMS Spectrum: m/z (ES+)(M+H)+=346; HPLC tR=1.77 min.

The preparation of 2-chloro-4-[(3S,5S)-3,5-dimethylmorpholin-4-yl]-6-(iodomethyl)pyrimidine was described earlier.

### EXAMPLE 93

### 1-[4-[4-[1-(Benzenesulfonyl)cyclopropyl]-6-[(3S,5S)-3,5-dimethylmorpholin-4-yl]pyrimidin-2-yl]phenyl]-3-cyclopropylurea

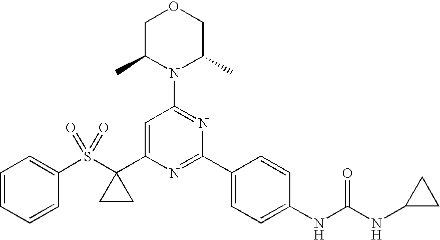

Cyclopropylamine (19.0 mg, 0.34 mmol) was added to phenyl N-[4-[4-[1-(benzenesulfonyl)cyclopropyl]-6-[(3S,5S)-3,5-dimethylmorpholin-4-yl]pyrimidin-2-yl]phenyl]carbamate (100 mg, 0.17 mmol) and triethylamine (52 mg, 0.51 mmol) in dioxane (10 mL) at RT. The resulting solution was stirred at 50° C. overnight. The crude product was purified by preparative HPLC, eluting with decreasingly polar mixtures of water (containing 1% ammonia) and acetonitrile, to give the desired material as a white solid (64 mg).

NMR Spectrum: ¹H NMR (400 MHz, DMSO-d₆) δ 0.43-0.39 (2H, m), 0.67-0.62 (2H, m), 1.28 (6H, d), 1.69-1.62 (2H, m), 1.93-1.89 (2H, m), 2.59-2.50 (m, 1H), 3.70-3.67 (2H, m), 4.18-4.10 (4H, m), 6.41 (1H, s), 6.60 (1H, s), 7.41 (2H, d), 7.59 (2H, t), 7.70 (1H, t), 7.79 (2H, d), 7.88 (2H, d), 8.49 (1H, s).

LCMS Spectrum: m/z (ES+)(M+H)+=548; HPLC tR=2.49 min.

The compounds below were prepared in an analogous fashion from phenyl N-[4-[4-[1-(benzenesulfonyl)cyclopropyl]-6-[(3S,5S)-3,5-dimethylmorpholin-4-yl]pyrimidin-2-yl]phenyl]carbamate and the appropriate amine.

| | | | | Retention |
|---|---|---|---|---|
| | | | LCMS | time |
| Example | Structure | NAME | MH+ | (min) |
| 93a | 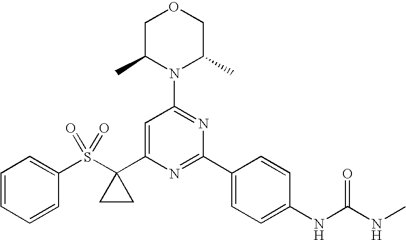 | 1-[4-[4-[1-(benzenesulfonyl)cyclopropyl]-6-[(3S,5S)-3,5-dimethylmorpholin-4-yl]pyrimidin-2-yl]phenyl]-3-methylurea | 522 | 2.32 |
| 93b | 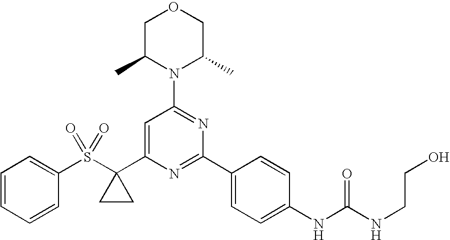 | 1-[4-[4-[1-(benzenesulfonyl)cyclopropyl]-6-[(3S,5S)-3,5-dimethylmorpholin-4-yl]pyrimidin-2-yl]phenyl]-3-(2-hydroxyethyl)urea | 552 | 2.13 |
| 93c | 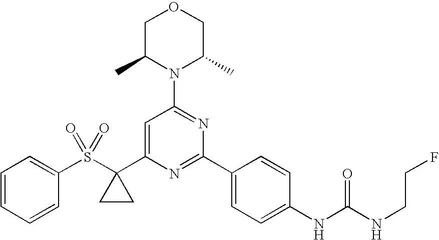 | 1-[4-[4-[1-(benzenesulfonyl)cyclopropyl]-6-[(3S,5S)-3,5-dimethylmorpholin-4-yl]pyrimidin-2-yl]phenyl]-3-(2-fluoroethyl)urea | 554 | 2.47 |
| 93d | 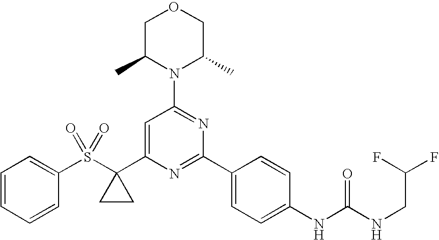 | 1-[4-[4-[1-(benzenesulfonyl)cyclopropyl]-6-[(3S,5S)-3,5-dimethylmorpholin-4-yl]pyrimidin-2-yl]phenyl]-3-(2,2-difluoroethyl)urea | 572 | 2.60 |
| 93e | 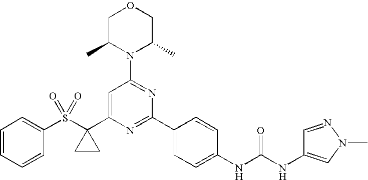 | 1-[4-[4-[1-(benzenesulfonyl)cyclopropyl]-6-[(3S,5S)-3,5-dimethylmorpholin-4-yl]pyrimidin-2-yl]phenyl]-3-(1-methylpyrazol-4-yl)urea | 588 | 2.34 |

1-[4-[4-[1-(Benzenesulfonyl)cyclopropyl]-6-[(3S,5S)-3,5-dimethylmorpholin-4-yl]pyrimidin-2-yl]phenyl]-3-ethylurea (Example 72) can also be prepared in this fashion.

### EXAMPLE 93a

¹H NMR (400.132 MHz, DMSO-d₆) δ 1.28 (6H, d), 1.69-1.62 (2H, m), 1.93-1.89 (2H, m), 2.68-2.65 (3H, m), 3.70-3.67 (2H, m), 4.16-4.10 (4H, m), 6.05 (1H, q), 6.59 (1H, s), 7.40 (2H, d), 7.59 (2H, t), 7.70 (1H, t), 7.79 (2H, d), 7.87 (2H, d), 8.69 (1H, s).

### EXAMPLE 93b

¹H NMR (400.132 MHz, DMSO-d₆) δ 1.28 (6H, d), 1.69-1.63 (2H, m), 1.93-1.89 (2H, m), 3.18-3.15 (2H, m), 3.46 (2H, q), 3.70-3.67 (2H, m), 4.18-4.10 (4H, m), 4.72 (1H, t), 6.24 (1H, t), 6.60 (1H, s), 7.39 (2H, d), 7.59 (2H, t), 7.70 (1H, t), 7.79 (2H, d), 7.88 (2H, d), 8.76 (1H, s).

### EXAMPLE 93c

¹H NMR (400.132 MHz, DMSO-d₆) δ 1.28 (6H, d), 1.69-1.64 (2H, m), 1.93-1.89 (2H, m), 3.41 (2H, dq), 3.70-3.68 (2H, m), 4.18-4.10 (4H, m), 4.47 (2H, dt), 6.42 (1H, t), 6.60 (1H, s), 7.40 (2H, d), 7.59 (2H, t), 7.70 (1H, t), 7.79 (2H, d), 7.88 (2H, d), 8.76 (1H, s).

### EXAMPLE 93d

¹H NMR (400.132 MHz, DMSO-d₆) δ 1.28 (6H, d), 1.69-1.61 (2H, m), 1.93-1.90 (2H, m), 3.59-3.49 (2H, m), 3.70-3.68 (2H, m), 4.18-4.10 (4H, m), 6.07 (1H, tt), 6.52 (1H, t), 6.60 (1H, s), 7.41 (2H, d), 7.59 (2H, t), 7.70 (1H, t), 7.79 (2H, d), 7.89 (2H, d), 8.87 (1H, s).

### EXAMPLE 93e

¹H NMR (400.132 MHz, DMSO-d₆) δ 1.28 (6H, d), 1.72-1.62 (2H, m), 1.94-1.90 (2H, m), 3.70-3.68 (2H, m), 3.79 (3H, s), 4.18-4.10 (4H, m), 6.61 (1H, s), 7.38 (1H, s), 7.45 (2H, d), 7.59 (2H, t), 7.71 (1H, t), 7.76 (1H, s), 7.80 (2H, d), 7.91 (2H, d), 8.37 (1H, s), 8.79 (1H, s).

The preparation of phenyl N-[4-[4-[1-(benzenesulfonyl)cyclopropyl]-6-[(3S,5S)-3,5-dimethylmorpholin-4-yl]pyrimidin-2-yl]phenyl]carbamate is described below.

### Phenyl N-[4-[4-[1-(benzenesulfonyl)cyclopropyl]-6-[(3S,5S)-3 5-dimethylmorpholin-4-yl]pyrimidin-2-yl]phenyl]carbamate

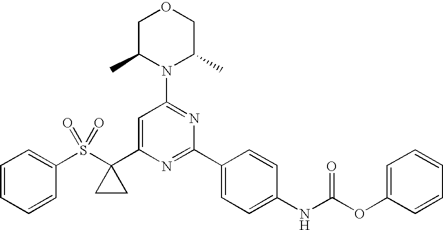

4-[4-[1-(Benzenesulfonyl)cyclopropyl]-6-[(3S,5S)-3,5-dimethylmorpholin-4-yl]pyrimidin-2-yl]aniline (0.6 g, 1.29 mmol) and sodium bicarbonate (1.085 g, 12.91 mmol) were added to DCM (60 mL) and stirred for 10 minutes. Phenyl chloroformate (0.211 mL, 1.68 mmol) was added slowly and the reaction was stirred for 1 hour. The reaction mixture was quenched with saturated ammonium chloride solution (50 mL), extracted with ethyl acetate (3×50 mL), the organic layer was dried over MgSO₄, filtered and evaporated to afford an orange solid. The crude product was purified by flash silica chromatography, elution gradient 30 to 60% ethyl acetate in isohexane, to give the desired material as a yellow solid (0.73 g).

NMR Spectrum: ¹H NMR (400 MHz, DMSO-d₆) δ 1.29 (6H, d), 1.71-1.64 (2H, m), 1.94-1.89 (2H, m), 3.71-3.68 (2H, m), 4.18-4.11 (4H, m), 6.63 (1H, s), 7.30-7.23 (3H, m), 7.45 (2H, t), 7.54 (2H, d), 7.59 (2H, t), 7.70 (1H, t), 7.81-7.79 (2H, m), 7.97 (2H, d), 10.39 (1H, s).

LCMS Spectrum: m/z (ES+)(M+H)+=585; HPLC tR=3.02 min.

### 4-[4-[1-(Benzenesulfonyl)cyclopropyl]-6-[(3S,5S)-3,5-dimethylmorpholin-4-yl]pyrimidin-2-yl]aniline

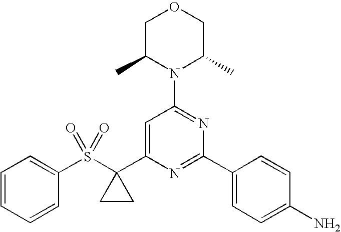

Bis(triphenylphosphine)palladium(II) chloride (0.053 g, 0.07 mmol) was added to 4-[1-(benzenesulfonyl)cyclopropyl]-2-chloro-6-[(3S,5S)-3,5-dimethylmorpholin-4-yl]pyrimidine (0.611 g, 1.50 mmol), 4-(4,4,5,5-tetramethyl-1,3,2-dioxaborolan-2-yl)aniline (0.427 g, 1.95 mmol) and 2M sodium carbonate solution (2.247 mL, 4.49 mmol) in water (3 mL), ethanol (3 mL), and DME (6 mL) and the resulting solution stirred at 95° C. overnight. The reaction mixture was diluted with ethyl acetate (75 mL), and washed sequentially with water (10 mL) and saturated brine (10 mL). The organic layer was dried over MgSO₄, filtered and evaporated to afford the crude product. The crude product was purified by flash silica chromatography, elution gradient 30 to 50% ethyl acetate in DCM, to give the desired material as a white foam (0.6 g).

NMR Spectrum: ¹H NMR (400 MHz, DMSO-d₆) δ 1.25 (6H, d), 1.69-1.60 (2H, m), 1.93-1.85 (2H, m), 3.68-3.64 (2H, m), 4.13-4.06 (4H, m), 5.50 (1H, s), 6.52-6.50 (4H, m), 7.58 (2H, t), 7.74-7.67 (3H, m), 7.80-7.78 (2H, m).

LCMS Spectrum: m/z (ES+)(M+H)+=465; HPLC tR=2.50 min.

### 4-[1-(Benzenesulfonyl)cyclopropyl]-2-chloro-6-[(3S,5S)-3,5-dimethylmorpholin-4-yl]pyrimidine

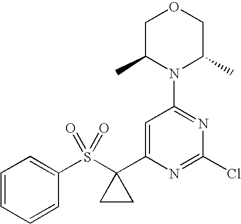

A solution of 50% w/v sodium hydroxide (4.99 mL, 124.73 mmol) was added portionwise to a stirred solution of 4-(benzenesulfonylmethyl)-2-chloro-6-[(3S,5S)-3,5-dimethylmorpholin-4-yl]pyrimidine (866 mg, 2.27 mmol), tetrabutylammonium bromide (73.1 mg, 0.23 mmol) and 1,2-dibromoethane (0.586 mL, 6.80 mmol) in toluene (50 mL) and the resulting suspension stirred at 60° C. for 6 hours. The reaction mixture was diluted with water (50 mL), and washed sequentially with water (2×50 mL), and saturated brine (50 mL). The organic layer was dried over MgSO₄, filtered and evaporated to afford crude product. The crude product was purified by flash silica chromatography, elution gradient 0 to 20% ethyl acetate in DCM, to give the desired material as a solid (611 mg).

NMR Spectrum: ¹H NMR (400 MHz, DMSO-d₆) δ 1.20 (6H, m), 1.59 (2H, m), 1.86 (2H, m), 3.66 (2H, dd), 4.02 (2H, m), 4.10 (2H, dd), 6.62 (1H, s), 7.61 (2H, m), 7.74 (1H, m), 7.75 (2H, m).

LCMS Spectrum: m/z (ES+)(M+H)+=408; HPLC tR=2.26 min.

### 4-(Benzenesulfonylmethyl)-2-chloro-6-[(3S,5S)-3,5-dimethylmorpholin-4-yl]pyrimidine

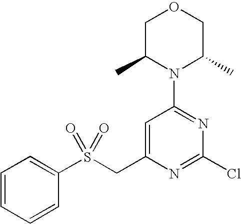

Sodium benzenesulfinate (0.872 g, 5.31 mmol) was added portionwise to 2-chloro-4-[(3S,5S)-3,5-dimethylmorpholin-4-yl]-6-(iodomethyl)pyrimidine (1.86 g, 5.06 mmol) in acetonitrile (50 mL) and the resulting suspension stirred at 80° C. for 6 hours. Further sodium benzenesulfinate (0.5 equivalents) was added and the reaction was heated for a further 8 hours. The solvent was removed and the solid was taken up in DMF. Further sodium benzenesulfinate (1 equivalent) was added and the reaction allowed to stir until complete (some sodium iodide was added to speed up the reaction). 10% Aqueous sodium thiosulfate solution was added and the acetonitrile removed, ethyl acetate was added and the layers separated. The organic layer was washed with brine and water, dried over MgSO₄, filtered and evaporated. The crude product was purified by flash silica chromatography, elution gradient 0 to 25% ethyl acetate in DCM, to give the desired material as a white solid (0.866 g).

NMR Spectrum: ¹H NMR (400 MHz, CDCl₃) δ 1.42-1.44(6H, d), 3.76-3.79(2H, dd), 4.10-4.16(2H, m), 4.22-4.26(2H, dd), 4.32(2H, s), 6.56(1H, s), 7.52-7.56(2H, t), 7.64-7.68(1H, t), 7.77-7.79(2H, d).

LCMS Spectrum: m/z (ES+)(M+H)+=382; HPLC tR=2.11 min.

The preparation of 2-chloro-4-[(3S,5S)-3,5-dimethylmorpholin-4-yl]-6-(iodomethyl)pyrimidine was described earlier.

### EXAMPLE 94

The following samples were prepared by heating a mixture of the carbamate (1 equivalent), triethylamine (4 equivalents) and the amine (4 equivalents) in NMP (2 mL) at 70° C. for 2 hours. The compounds were purified by preparative HPLC.

The following compounds were prepared in an analogous fashion from either phenyl N-[4-[4-[(3S)-3-methylmorpholin-4-yl]-6-[1-(oxan-4-ylsulfonyl)cyclopropyl]pyrimidin-2-yl]phenyl]carbamate or phenyl N-[4-[4-[(3S)-3-methylmorpholin-4-yl]-6-[1-(oxolan-3-ylsulfonyl)cyclopropyl]pyrimidin-2-yl]phenyl]carbamate and the appropriate amine.

| | | | | Retention |
|---|---|---|---|---|
| Ex- | | | LCMS | time |
| ample | Structure | NAME | MH+ | (min) |
| 94a | 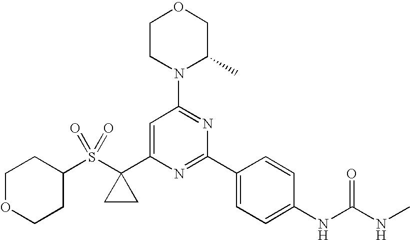 | 3-methyl-1-[4-[4-[(3S)-3-methylmorpholin-4-yl]-6-[1-(oxan-4-ylsulfonyl)cyclopropyl]pyrimidin-2-yl]phenyl]urea | 516 | 1.98 |
| 94b | 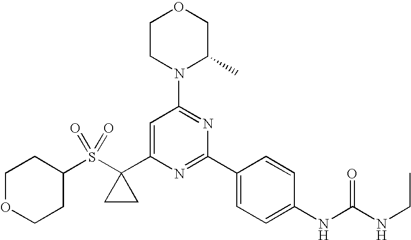 | 1-ethyl-3-[4-[4-[(3S)-3-methylmorpholin-4-yl]-6-[1-(oxan-4-ylsulfonyl)cyclopropyl]pyrimidin-2-yl]phenyl]urea | 530 | 2.14 |
| 94c | 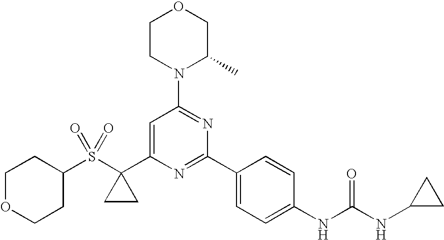 | 3-cyclopropyl-1-[4-[4-[(3S)-3-methylmorpholin-4-yl]-6-[1-(oxan-4-ylsulfonyl)cyclopropyl]pyrimidin-2-yl]phenyl]urea | 542 | 2.16 |
| 94d | 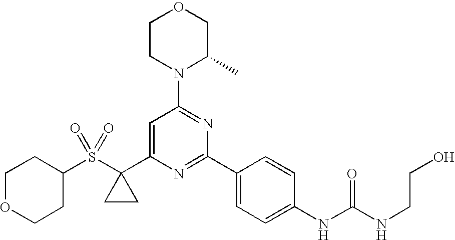 | 3-(2-hydroxyethyl)-1-[4-[4-[(3S)-3-methylmorpholin-4-yl]-6-[1-(oxan-4-ylsulfonyl)cyclopropyl]pyrimidin-2-yl]phenyl]urea | 546 | 1.80 |
| 94e | 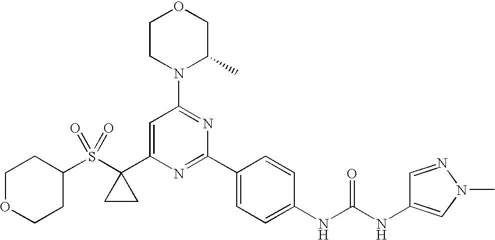 | 1-[4-[4-[(3S)-3-methylmorpholin-4-yl]-6-[1-(oxan-4-ylsulfonyl)cyclopropyl]pyrimidin-2-yl]phenyl]-3-(1-methylpyrazol-4-yl)urea | 582 | 2.03 |
| 94f | 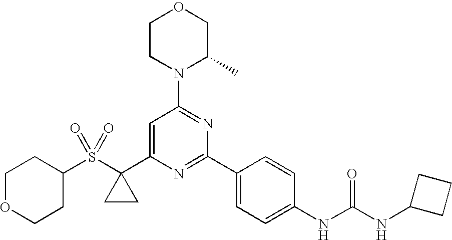 | 3-cyclobutyl-1-[4-[4-[(3S)-3-methylmorpholin-4-yl]-6-[1-(oxan-4-ylsulfonyl)cyclopropyl]pyrimidin-2-yl]phenyl]urea | 556 | 2.44 |
| 94g | 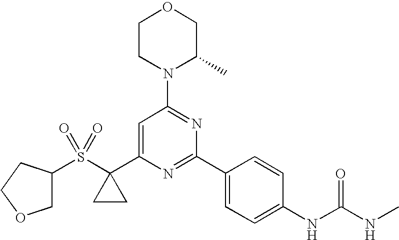 | 3-methyl-1-[4-[4-[(3S)-3-methylmorpholin-4-yl]-6-[1-(oxolan-3-ylsulfonyl)cyclopropyl]pyrimidin-2-yl]phenyl]urea | 502 | 1.98 |
| 94h | 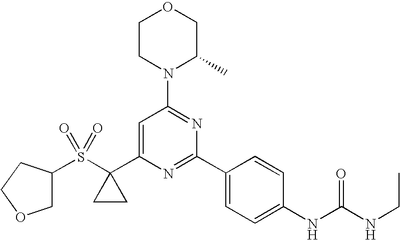 | 1-ethyl-3-[4-[4-[(3S)-3-methylmorpholin-4-yl]-6-[1-(oxolan-3-ylsulfonyl)cyclopropyl]pyrimidin-2-yl]phenyl]urea | 516 | 2.13 |
| 94i | 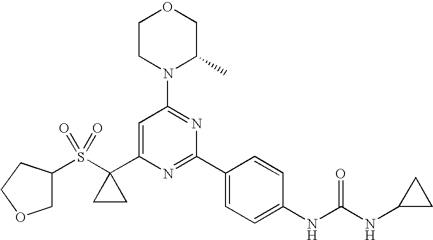 | 3-cyclopropyl-1-[4-[4-[(3S)-3-methylmorpholin-4-yl]-6-[1-(oxolan-3-ylsulfonyl)cyclopropyl]pyrimidin-2-yl]phenyl]urea | 528 | 2.15 |
| 94j | 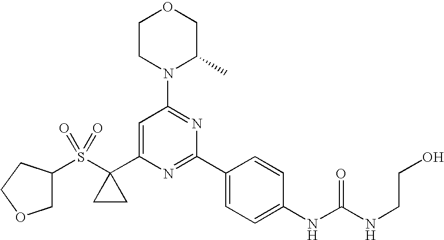 | 3-(2-hydroxyethyl)-1-[4-[4-[(3S)-3-methylmorpholin-4-yl]-6-[1-(oxolan-3-ylsulfonyl)cyclopropyl]pyrimidin-2-yl]phenyl]urea | 532 | 1.81 |
| 94k | 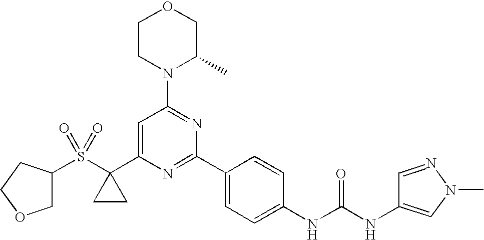 | 1-[4-[4-[(3S)-3-methylmorpholin-4-yl]-6-[1-(oxolan-3-ylsulfonyl)cyclopropyl]pyrimidin-2-yl]phenyl]-3-(1-methylpyrazol-4-yl)urea | 568 | 2.03 |

### EXAMPLE 94a

¹H NMR (400.132 MHz, DMSO-d₆) δ 1.24 (3H, d), 1.52-1.56 (2H, m), 1.58-1.61 (2H, m), 1.65-1.77 (2H, m), 2.10-2.19 (2H, m), 3.17-3.33 (3H, m), 3.48 (1H, td), 3.63 (1H, d), 3.75-3.87 (2H, m), 3.95-4.05 (3H, m), 4.17-4.27 (1H, m), 4.55 (1H, s), 6.00-6.15 (1H, m), 6.78 (1H, s), 7.52 (2H, d), 8.18 (2H, d), 8.75 (1H, s)

### EXAMPLE 94b

¹H NMR (400.132 MHz, DMSO-d₆) δ 1.07 (3H, t), 1.24 (3H, d), 1.52-1.56 (2H, m), 1.57-1.62 (2H, m), 1.65-1.78 (2H, m), 2.09-2.19 (2H, m), 3.09-3.24 (3H, m), 3.26-3.35 (2H, m), 3.48 (1H, td), 3.63 (1H, d), 3.74-3.88 (2H, m), 3.94-4.04 (3H, m), 4.22 (1H, d), 4.55 (1H, s), 6.17 (1H, t), 6.78 (1H, s), 7.51 (2H, d), 8.18 (2H, d), 8.67 (1H, s)

### EXAMPLE 94c

¹H NMR (400.132 MHz, DMSO-d₆) δ 0.38-0.46 (2H, m), 0.62-0.69 (2H, m), 1.24 (3H, d), 1.50-1.56 (2H, m), 1.56-1.62 (2H, m), 1.66-1.78 (2H, m), 2.09-2.19 (2H, m), 2.54-2.60 (1H, m), 3.21 (1H, td), 3.28-3.34 (2H, m), 3.48 (1H, td), 3.63 (1H, d), 3.76 (1H, d), 3.79-3.88 (1H, m), 3.94-4.04 (3H, m), 4.22 (1H, d), 4.55 (1H, s), 6.45 (1H, s), 6.78 (1H, s), 7.52 (2H, d), 8.19 (2H, d), 8.55 (1H, s)

### EXAMPLE 94d

¹H NMR (400.132 MHz, DMSO-d₆) δ 1.24 (3H, d), 1.50-1.56 (2H, m), 1.56-1.62 (2H, m), 1.64-1.77 (2H, m), 2.09-2.20 (2H, m), 3.15-3.23 (2H, m), 3.25-3.35 (2H, m), 3.43-3.50 (2H, m), 3.61-3.66 (1H, m), 3.74-3.87 (2H, m), 3.94-4.04 (3H, m), 4.22 (1H, d), 4.55 (1H, s), 4.73 (1H, t), 6.27 (1H, t), 6.78 (1H, s), 7.50 (2H, d), 8.18 (2H, d), 8.82 (1H, s)

### EXAMPLE 94e

¹H NMR (400.132 MHz, DMSO-d₆) δ 1.24 (3H, d), 1.51-1.57 (2H, m), 1.58-1.62 (2H, m), 1.67-1.78 (2H, m), 2.10-2.21 (2H, m), 3.18-3.34 (3H, m), 3.49 (1H, td), 3.64 (1H, d), 3.73-3.88 (5H, m), 3.95-4.05 (3H, m), 4.23 (1H, d), 4.55 (1H, s), 6.79 (1H, s), 7.39 (1H, s), 7.56 (2H, d), 7.77 (1H, s), 8.22 (2H, d), 8.40 (1H, s), 8.85 (1H, s)

### EXAMPLE 94f

¹H NMR (400.132 MHz, DMSO-d₆) δ 1.23 (3H, d), 1.49-1.77 (8H, m), 1.81-1.91 (2H, m), 2.09-2.25 (4H, m), 3.21 (1H, td), 3.26-3.35 (2H, m), 3.48 (1H, td), 3.63 (1H, d), 3.71-3.89 (2H, m), 3.94-4.04 (3H, m), 4.08-4.25 (2H, m), 4.55 (1H, s), 6.47 (1H, d), 6.78 (1H, s), 7.49 (2H, d), 8.18 (2H, d), 8.57 (1H, s)

### EXAMPLE 94g

¹H NMR (400.132 MHz, DMSO-d₆) δ 1.23 (3H, d), 1.52-1.58 (2H, m), 1.62-1.70 (2H, m), 2.17-2.30 (2H, m), 2.66 (3H, d), 3.14-3.28 (1H, m), 3.49 (1H, td), 3.61-3.72 (2H, m), 3.73-3.82 (2H, m), 3.93-4.04 (2H, m), 4.21 (1H, d), 4.27-4.39 (1H, m), 4.55 (1H, s), 6.07 (1H, t), 6.79 (1H, s), 7.51 (2H, d), 8.20 (2H, d), 8.74 (1H, s)

### EXAMPLE 94h

¹H NMR (400.132 MHz, DMSO-d₆) δ 1.07 (3H, t), 1.23 (3H, d), 1.52-1.59 (2H, m), 1.63-1.69 (2H, m), 2.18-2.30 (2H, m), 3.09-3.17 (2H, m), 3.17-3.24 (1H, m), 3.49 (1H, td), 3.61-3.71 (2H, m), 3.73-3.83 (2H, m), 3.94-4.02 (3H, m), 4.21 (1H, d), 4.30-4.38 (1H, m), 4.54 (1H, s), 6.17 (1H, t), 6.79 (1H, s), 7.50 (2H, d), 8.20 (2H, d), 8.66 (1H, s)

### EXAMPLE 94i

¹H NMR (400.132 MHz, DMSO-d₆) δ 0.40-0.44 (2H, m), 0.62-0.68 (3H, m), 1.23 (4H, d), 1.52-1.60 (4H, m), 1.63-1.70 (3H, m), 2.16-2.30 (3H, m), 2.52-2.61 (18H, m), 3.21 (3H, td), 3.49 (1H, td), 3.59-3.72 (3H, m), 3.72-3.84 (3H, m), 3.93-4.03 (3H, m), 4.21 (1H, d), 4.28-4.40 (1H, m), 4.54 (1H, s), 6.44 (1H, s), 6.80 (1H, s), 7.52 (2H, d), 8.20 (2H, d), 8.54 (1H, s)

### EXAMPLE 94j

¹H NMR (400.132 MHz, DMSO-d₆) δ 1.23 (3H, d), 1.51-1.60 (2H, m), 1.62-1.69 (2H, m), 2.18-2.30 (2H, m), 3.14-3.24 (4H, m), 3.43-3.50 (2H, m), 3.61-3.71 (1H, m), 3.74-3.81 (2H, m), 3.94-4.02 (3H, m), 4.21 (1H, d), 4.30-4.38 (1H, m), 4.54 (1H, s), 4.69-4.77 (1H, m), 6.26 (1H, t), 6.79 (1H, s), 7.50 (2H, d), 8.20 (2H, d), 8.81 (1H, s)

### EXAMPLE 94k

¹H NMR (400.132 MHz, DMSO-d₆) δ 1.24 (3H, d), 1.50-1.60 (2H, m), 1.63-1.68 (2H, m), 2.20-2.30 (2H, m), 3.16-3.28 (1H, m), 3.44-3.57 (1H, m), 3.61-3.70 (1H, m), 3.74-3.84 (4H, m), 3.94-4.05 (3H, m), 4.22 (1H, d), 4.31-4.38 (1H, m), 4.55 (1H, s), 6.81 (1H, s), 7.39 (1H, s), 7.56 (2H, d), 7.76 (1H, s), 8.24 (2H, d), 8.39 (1H, s), 8.84 (1H, s)

The preparation of phenyl N-[4-[4-[(3S)-3-methylmorpholin-4-yl]-6-[1-(oxan-4-ylsulfonyl)cyclopropyl]pyrimidin-2-yl]phenyl]carbamate is described below.

### Phenyl N-[4-[4-[(3S)-3-methylmorpholin-4-yl]-6-[1-(oxan-4-ylsulfonyl)cyclopropyl]pyrimidin-2-yl]phenyl]carbamate

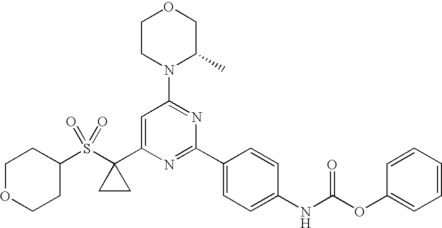

Phenyl chloroformate (0.436 mL, 3.47 mmol) was added to 4-[4-[(3S)-3-methylmorpholin-4-yl]-6-[1-(oxan-4-ylsulfonyl)cyclopropyl]pyrimidin-2-yl]aniline (1.06 g, 2.31 mmol), sodium hydrogen carbonate (0.291 g, 3.47 mmol) in dioxane (40 mL) at 5° C. under a nitrogen atmosphere. The resulting mixture was stirred at RT for 1.5 hours. The reaction mixture was diluted with ethyl acetate (150 mL), and washed sequentially with water (2×100 mL). The organic layer was dried over Na₂SO₄, filtered and evaporated to afford crude product which was triturated with a mixture of diethyl ether and isohexane to give the desired material as a beige solid (1.14 g).

NMR Spectrum: ¹H NMR (400.132 MHz, DMSO-d₆) δ 1.24 (3H, d), 1.52-1.58 (2H, m), 1.58-1.64 (2H, m), 1.65-1.77 (2H, m), 2.10-2.20 (2H, m), 3.17-3.54 (2H, m), 3.63 (2H, d), 3.74-3.87 (3H, m), 3.96-4.04 (3H, m), 4.24 (1H, d), 4.56 (1H, s), 6.83 (1H, s), 7.20-7.32 (3H, m), 7.42-7.50 (2H, m), 7.63 (2H, d), 8.28 (2H, d), 10.45 (1H, s)

LCMS Spectrum: m/z (ESI+)(M+H)+=579; HPLC tR=2.8 min.

### 4-[4-[(3S)-3-Methylmorpholin-4-yl]-6-[1-(oxan-4-ylsulfonyl)cyclopropyl]pyrimidin-2-yl]aniline

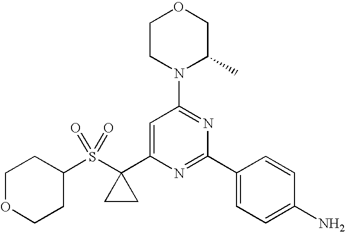

Bis(triphenylphosphine)palladium(II) chloride (0.187 g, 0.27 mmol) was added to 2-chloro-4-[(3S)-3-methylmorpholin-4-yl]-6-[1-(oxan-4-ylsulfonyl)cyclopropyl]pyrimidine (1.6 g, 3.98 mmol), 4-(4,4,5,5-tetramethyl-1,3,2-dioxaborolan-2-yl)aniline (1.308 g, 5.97 mmol) and 2M aqueous sodium carbonate solution (1 mL, 2.00 mmol) in a solvent mixture of DMF (5 mL), DME (12 mL), water (1 mL) and ethanol (1 mL) at RT . The resulting mixture was stirred at 90° C. for 7 hours under an inert atmosphere. The reaction mixture was diluted with ethyl acetate (100 mL), and washed sequentially with water (2×100 mL). The organic layer was dried over Na₂SO₄, filtered and evaporated to afford crude product. The crude product was purified by flash silica chromatography, elution gradient 10 to 50% ethyl acetate in isohexane, and the crude product then further purified by ion exchange chromatography using an SCX column, eluting with 7M ammonia in methanol, to give a solid. The solid was further purified by trituration with a mixture of diethyl ether and isohexane to give the desired material as a beige solid (1.0 g).

NMR Spectrum: ¹H NMR (400.132 MHz, DMSO-d₆) δ 1.22 (3H, d), 1.48-1.53 (2H, m), 1.55-1.59 (2H, m), 1.66-1.76 (2H, m), 2.11-2.18 (2H, m), 3.18 (1H, dd), 3.30-3.34 (2H, m), 3.47 (1H, td), 3.62 (1H, d), 3.75 (1H, d), 3.81-3.90 (1H, m), 3.93-4.05 (3H, m), 4.18 (1H, d), 4.51 (1H, s), 5.58 (2H, s), 6.62 (2H, d), 6.67 (1H, s), 8.02 (2H, d)

LCMS Spectrum: m/z (ESI+)(M+H)+=459; HPLC tR=2.11 min.

### 2-Chloro-4-[(3S)-3-methylmorpholin-4-yl]-6-[1-(oxan-4-ylsulfonyl)cyclopropyl]pyrimidine

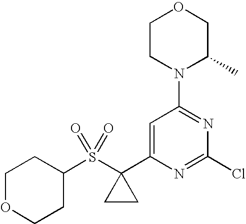

Aqueous sodium hydroxide solution (10 mL, 186.39 mmol) was added to 2-chloro-4-[(3S)-3-methylmorpholin-4-yl]-6-(oxan-4-ylsulfonylmethyl)pyrimidine (1.5 g, 3.99 mmol), tetraethylammonium bromide (0.168 g, 0.80 mmol) and 1,2-dibromoethane (2.75 mL, 31.93 mmol) in toluene (10 mL) at RT under a nitrogen atmosphere. After stirring at RT for 2 hours the reaction mixture was diluted with ethyl acetate (200 mL), and washed with water (100 mL). The organic layer was dried over Na₂SO₄, filtered and evaporated to afford crude product. The crude product was purified by flash silica chromatography, elution gradient 5 to 60% ethyl acetate in isohexane, to give the desired material as an oil which solidified on standing (1.64 g).

NMR Spectrum: ¹H NMR (399.902 MHz, DMSO-d₆) δ 1.20-1.25 (3H, m), 1.50-1.54 (2H, m), 1.55-1.59 (2H, m), 1.61-1.72 (2H, m), 1.97-2.07 (2H, m), 3.18-3.27 (1H, m), 3.26-3.36 (2H, m), 3.44 (1H, td), 3.58 (1H, d), 3.65-3.78 (2H, m), 3.90-4.01 (3H, m), 4.01-4.10 (1H, m), 4.39 (1H, s), 6.96 (1H, s)

LCMS Spectrum: m/z (ESI+)(M+H)+=402; HPLC tR=1.99 min.

### 2-Chloro-4-[(3S)-3-methylmorpholin-4-yl]-6-(oxan-4-ylsulfonylmethyl)pyrimidine

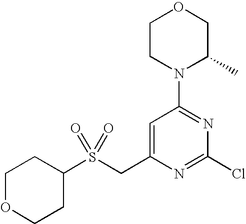

3-Chloroperoxybenzoic acid (381 mg, 2.21 mmol) was added to 2-chloro-4-[(3S)-3-methylmorpholin-4-yl]-6-(oxan-4-ylsulfanylmethyl)pyrimidine (345 mg, 1.00 mmol)in DCM (10 mL) at RT under a nitrogen atmosphere. The resulting solution was stirred at RT for 2 hours then diluted with DCM (100 mL), and washed sequentially with 10% aqueous sodium metabisulphite solution (200 mL), saturated aqueous sodium hydrogencarbonate (200 mL), dried over Na₂SO₄, filtered and evaporated to afford crude product. The crude solid was triturated with a mixture of diethyl ether and isohexane to give the desired material as a cream solid (200 mg).

NMR Spectrum: ¹H NMR (399.902 MHz, DMSO-d₆) δ 1.19-1.26 (3H, m), 1.60-1.76 (2H, m), 1.95-2.06 (2H, m), 3.19-3.29 (1H, m), 3.31-3.39 (2H, m), 3.40-3.65 (3H, m), 3.74 (1H, d), 3.90-4.04 (3H, m), 4.30 (1H, s), 4.48 (2H, s), 6.93 (1H, s)

### 2-Chloro-4-[(3S)-3-methylmorpholin-4-yl]-6-(oxan-4-ylsulfanylmethyl)pyrimidine

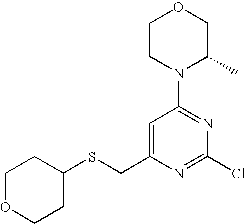

DIPEA (1.762 mL, 10.18 mmol) was added to oxane-4-thiol (1.203 g, 10.18 mmol), in acetonitrile (20 mL) at RT under a nitrogen atmosphere. The resulting solution was stirred at RT for 5 mins then 2-chloro-4-(iodomethyl)-6-[(3S)-3-methylmorpholin-4-yl]pyrimidine (2.4 g, 6.79 mmol) added. The resulting mixture was stirred at RT for 1 hour then diluted with ethyl acetate (75 mL), and washed sequentially with water (2×75 mL). The organic layer was dried over MgSO₄, filtered and evaporated to afford crude product. The crude product was purified by flash silica chromatography, elution gradient 10 to 70% ethyl acetate in isohexane, to give the desired material as a brown gum (2.5 g).

NMR Spectrum: ¹H NMR (400.132 MHz, DMSO-d₆) δ 1.19 (3H, d), 1.39-1.51 (2H, m), 1.82-1.92 (2H, m), 2.92-3.02 (1H, m), 3.13-3.23 (1H, m), 3.26-3.37 (2H, m), 3.44 (1H, td), 3.54-3.66 (1H, m), 3.72 (1H, d), 3.78-3.88 (2H, m), 3.90-4.02 (2H, m), 4.31 (1H, s), 6.81 (1H, s)

LCMS Spectrum: m/z (ESI+)(M+H)+=344; HPLC tR=1.99 min.

The preparation of 2-chloro-4-(iodomethyl)-6-[(3S)-3-methylmorpholin-4-yl]pyrimidine was described earlier.

### Oxane-4-thiol

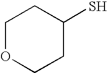

7M Ammonia in methanol (20 mL, 140.00 mmol) was added to S-(oxan-4-yl)ethanethioate (2.27 g, 14.17 mmol) at RT. The resulting solution was stirred at RT for 1 hour then the mixture concentrated in vacuo and used without further purification.

NMR Spectrum: none

LCMS Spectrum: no mass ion; HPLC tR=0.61 min.

### S-(Oxan-4-yl) ethanethioate

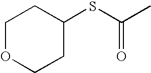

Potassium thioacetate (4.68 g, 40.98 mmol) was added to oxan-4-yl methanesulfonate (4.2 g, 23.30 mmol), in DMA (80 mL) at RT. The resulting mixture was stirred at 65° C. for 18 hours. The reaction mixture was diluted with ethyl acetate (400 mL), and washed sequentially with water (2×150 mL). The organic layer was dried over Na₂SO₄, filtered and evaporated to afford crude product. The crude product was purified by flash silica chromatography, elution gradient 5 to 30% ethyl acetate in isohexane, to give the desired material as a orange oil (2.27 g).

NMR Spectrum: ¹H NMR (400.132 MHz, DMSO-d₆) δ 1.47-1.65 (2H, m), 1.76-1.90 (2H, m), 2.35 (3H, s), 3.36-3.51 (2H, m), 3.53-3.69 (1H, m), 3.67-3.82 (2H, m).

LCMS Spectrum: no mass ion; HPLC tR=1.45 min.

The preparation of phenyl N-[4-[4-[(3S)-3-methylmorpholin-4-yl]-6-[1-(oxolan-3-ylsulfonyl)cyclopropyl]pyrimidin-2-yl]phenyl]carbamate is described below.

### Phenyl N-[4-[4-[(3S)-3-methylmorpholin-4-yl]-6-[1-(oxolan-3-ylsulfonyl)cyclopropyl]pyrimidin-2-yl]phenyl]carbamate

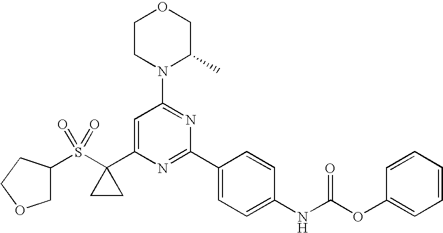

Phenyl chloroformate (0.297 mL, 2.36 mmol) was added to 4-[4-[(3S)-3-methylmorpholin-4-yl]-6-[1-(oxolan-3-ylsulfonyl)cyclopropyl]pyrimidin-2-yl]aniline (700 mg, 1.57 mmol), sodium hydrogen carbonate (198 mg, 2.36 mmol) in dioxane (30 mL) at 50° C. under a nitrogen atmosphere. The resulting mixture was stirred at RT for 1.5 hours then diluted with ethyl acetate (150 mL), and washed sequentially with water (2×100 mL). The organic layer was dried over Na₂SO₄, filtered and evaporated to afford crude product. The crude gum was triturated with a mixture of diethyl ether and isohexane to give the desired material as a beige solid (600 mg).

NMR Spectrum: ¹H NMR (400.132 MHz, DMSO-d₆) δ 1.25 (3H, d), 1.52-1.59 (2H, m), 1.64-1.71 (2H, m), 2.15-2.30 (2H, m), 3.22 (1H, td), 3.44-3.54 (1H, m), 3.61-3.70 (2H, m), 3.73-3.83 (2H, m), 3.94-4.03 (3H, m), 4.23 (1H, d), 4.30-4.38 (1H, m), 4.56 (1H, s), 6.84 (1H, s), 7.21-7.32 (3H, m), 7.41-7.50 (2H, m), 7.64 (2H, d), 8.30 (2H, d), 10.44 (1H, s)

### 4-[4-[(3S)-3-Methylmorpholin-4-yl]-6-[1-(oxolan-3-ylsulfonyl)cyclopropyl]pyrimidin-2-yl]aniline

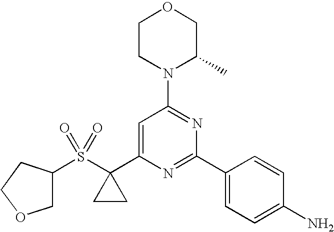

Bis(triphenylphosphine)palladium(II) chloride (0.233 g, 0.33 mmol) was added to 2-chloro-4-[(3S)-3-methylmorpholin-4-yl]-6-[1-(oxolan-3-ylsulfonyl)cyclopropyl]pyrimidine (1.92 g, 4.95 mmol), 4-(4,4,5,5-tetramethyl-1,3,2-dioxaborolan-2-yl)aniline (1.627 g, 7.42 mmol) and sodium carbonate (1 mL, 2.00 mmol) in a solvent mixture of DMF (5 mL), DME (12 mL), water (1 mL) and ethanol (1 mL) at RT. The resulting mixture was stirred at 90° C. for 7 hours under an inert atmosphere. The reaction mixture was diluted with ethyl acetate (100 mL), and washed sequentially with water (2×100 mL).The organic layer was dried over Na₂SO₄, filtered and evaporated to afford crude product. The crude product was purified by flash silica chromatography, elution gradient 10 to 50% ethyl acetate in isohexane, to give a product that was further purified by ion exchange chromatography using an SCX column, eluting with 7M ammonia in methanol, to give a solid. The crude solid was triturated with a mixture of diethyl ether and isohexane to give the desired material as a beige solid (0.821 g).

NMR Spectrum: ¹H NMR (400.132 MHz, DMSO-d₆) δ 1.22 (3H, d), 1.49-1.54 (2H, m), 1.61-1.66 (2H, m), 2.16-2.26 (2H, m), 3.18 (1H, dd), 3.39-3.53 (1H, m), 3.59-3.71 (3H, m), 3.70-3.83 (2H, m), 3.90-4.01 (3H, m), 4.17 (1H, d), 4.33 (1H, q), 4.51 (1H, s), 5.57 (2H, s), 6.61 (2H, d), 6.69 (1H, s), 8.03 (2H, d)

LCMS Spectrum: m/z (ESI+)(M+H)+=447; HPLC tR=1.91 min.

### 2-Chloro-4-[(3S)-3-methylmorpholin-4-yl]-6-[1-(oxolan-3-ylsulfonyl)cyclopropyl]pyrimidine

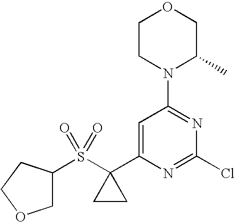

Aqueous sodium hydroxide solution (10 mL, 186.39 mmol) was added to 2-chloro-4-[(3S)-3-methylmorpholin-4-yl]-6-(oxolan-3-ylsulfonylmethyl)pyrimidine (1.9 g, 5.25 mmol), tetraethylammonium bromide (0.221 g, 1.05 mmol), and 1,2-dibromoethane (3.62 mL, 42.01 mmol) in toluene (30 mL) at RT under a nitrogen atmosphere. After being stirred at RT for 1.5 hours the reaction mixture was diluted with ethyl acetate (200 mL), and washed with water (125 mL). The organic layer was dried over Na₂SO₄, filtered and evaporated to afford crude product. The crude product was purified by flash silica chromatography, elution gradient 10 to 60% ethyl acetate in isohexane, to give the desired material as a colourless gum (1.92 g).

NMR Spectrum: none

LCMS Spectrum: m/z (ESI+)(M+H)+=388; HPLC tR=1.91 min.

### 2-Chloro-4-[(3S)-3-methylmorpholin-4-yl]-6-(oxolan-3-ylsulfonylmethyl)pyrimidine

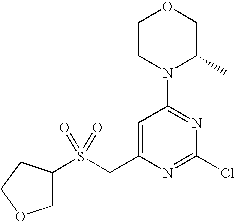

3-Chloroperoxybenzoic acid (4.26 g, 24.68 mmol) was added to 2-chloro-4-[(3S)-3-methylmorpholin-4-yl]-6-(oxolan-3-ylsulfanylmethyl)pyrimidine (3.7 g, 11.22 mmol)in DCM (200 mL) at RT under a nitrogen atmosphere. The resulting solution was stirred at RT for 2 hours then diluted with DCM (100 mL), and washed sequentially with 10% aqueous sodium metabisulphite solution (200 mL) and a saturated aqueous solution of sodium hydrogencarbonate (200 mL). The organic layer was dried over Na₂SO₄, filtered and evaporated to afford crude product. The crude solid was triturated with a mixture of diethyl ether and isohexane to give the desired material as a cream solid (3.03 g).

NMR Spectrum: ¹H NMR (399.902 MHz, DMSO-d₆) δ 1.20-1.26 (3H, m), 2.18-2.31 (2H, m), 3.20-3.31 (1H, m), 3.37-3.52 (1H, m), 3.60 (1H, d), 3.65-3.77 (2H, m), 3.80-3.89 (1H, m), 3.90-3.99 (3H, m), 4.03-4.16 (2H, m), 4.31 (1H, s), 4.49 (2H, s), 6.94 (1H, s)

LCMS Spectrum: m/z (ESI+)(M+H)+=362; HPLC tR=1.59 min.

### 2-Chloro-4-[(3S)-3-methylmorpholin-4-yl]-6-(oxolan-3-ylsulfanylmethyl)pyrimidine

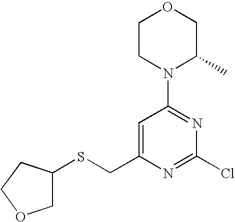

DIPEA (3.67 mL, 21.21 mmol) was added to oxolane-3-thiol (2.210 g, 21.21 mmol), in acetonitrile (100 mL) at RT under a nitrogen atmosphere. The resulting solution was stirred for 5 minutes then 2-chloro-4-(iodomethyl)-6-[(3S)-3-methylmorpholin-4-yl]pyrimidine (5 g, 14.14 mmol) added. The resulting mixture was stirred at RT for 1 hour then diluted with ethyl acetate (75 mL), and washed sequentially with water (2×75 mL). The organic layer was dried over MgSO₄, filtered and evaporated to afford crude product. The crude product was purified by flash silica chromatography, elution gradient 10 to 70% ethyl acetate in isohexane, to give the desired material as a brown gum (3.75 g).

NMR Spectrum: ¹H NMR (399.902 MHz, DMSO-d₆) δ 1.17-1.24 (3H, m), 1.63-1.75 (2H, m), 2.17-2.30 (2H, m), 3.19 (1H, td), 3.40-3.50 (2H, m), 3.59 (1H, d), 3.63-3.80 (3H, m), 3.91-4.04 (4H, m), 4.33 (1H, s), 6.82 (1H, s)

LCMS Spectrum: m/z (ESI+)(M+H)+=330; HPLC tR=1.89 min.

The preparation of 2-chloro-4-(iodomethyl)-6-[(3S)-3-methylmorpholin-4-yl]pyrimidine was described earlier.

### Oxolane-3-thiol

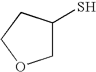

7M ammonia in methanol (20 mL, 140.00 mmol) was added to S-(Oxolan-3-yl)ethanethioate (20.47 g, 140.00 mmol) at RT. The resulting solution was stirred at RT for 1 hour then concentrated in vacuo to give the desired material which was used without further purification or characterisation.

S-(Oxolan-3-yl)ethanethioate

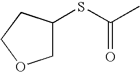

Potassium thioacetate (16.60 g, 145.31 mmol) was added to oxolan-3-yl methanesulfonate (13.8 g, 83.03 mmol), in DMA (150 mL) at RT. The resulting mixture was stirred at 65° C. for 7 hours. The reaction mixture was diluted with ethyl acetate (400 mL), and washed sequentially with water (2×150 mL). The organic layer was dried over Na₂SO₄, filtered and evaporated to afford crude product. The crude product was purified by flash silica chromatography, elution gradient 0 to 30% ethyl acetate in isohexane, to give the desired material as a brown oil (9.50 g).

NMR Spectrum: ¹H NMR (400.132 MHz, DMSO-d₆) δ 1.70-1.80 (1H, m), 2.25-2.40 (4H, m), 3.44-3.52 (1H, m), 3.67-3.81 (2H, m), 3.84-3.94 (1H, m), 3.97-4.08 (1H, m)

LCMS Spectrum: no mass ion; HPLC tR=1.18 min.

### Oxolan-3-yl methanesulfonate

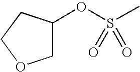

Methanesulfonyl chloride (11.86 mL, 153.23 mmol) was added dropwise to tetrahydrofuran-3-ol (9 g, 102.15 mmol) and triethylamine (21.36 mL, 153.23 mmol) in DCM (300 mL) at RT over a period of 30 minutes under a nitrogen atmosphere. The resulting mixture was stirred at RT for 1 hour then diluted with ethyl acetate (400 mL) and washed with water (250 mL). The organic layer was dried over Na₂SO₄, filtered and evaporated to afford the desired material, which was used without further purification.

NMR Spectrum: ¹H NMR (400.132 MHz, DMSO-d₆) δ 2.03-2.14 (1H, m), 2.17-2.29 (1H, m), 3.21 (3H, s), 3.69-3.92 (4H, m), 5.28-5.33 (1H, m)

### EXAMPLE 95

The following samples were prepared by heating a mixture of the carbamate (1 equivalent), triethylamine (4 equivalents) and the amine (1.2 equivalents) in NMP (2 mL) at 50° C. for 2 hours. The compounds were purified by preparative HPLC.

The following compounds were prepared in an analogous fashion from either phenyl N-[4-[4-[1-(3-chloro-4-fluorophenyl)sulfonylcyclopropyl]-6-[(3S)-3-methylmorpholin-4-yl]pyrimidin-2-yl]phenyl]carbamate or phenyl N-[4-[4-[(3S)-3-methylmorpholin-4-yl]-6-[1-(4-methylphenyl)sulfonylcyclopropyl]pyrimidin-2-yl]phenyl]carbamate and the appropriate amine.

| | | | | Retention |
|---|---|---|---|---|
| Ex- | | | LCMS | time |
| ample | Structure | NAME | MH+ | (min) |
| 95a | 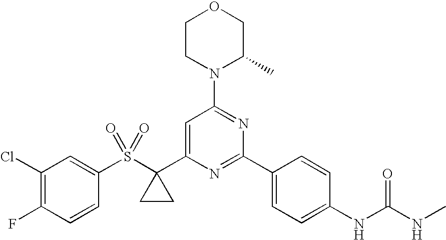 | 1-[4-[4-[1-(3-chloro-4-fluorophenyl)sulfonyl-cyclopropyl]-6-[(3S)-3-methylmorpholin-4-yl]pyrimidin-2-yl]phenyl]-3-methylurea | 560 | 2.29 |
| 95b | 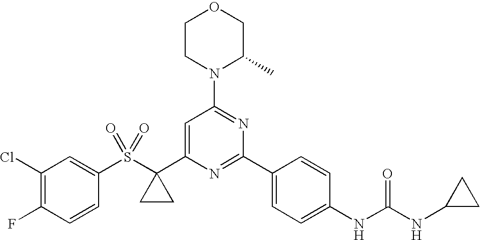 | 1-[4-[4-[1-(3-chloro-4-fluorophenyl)sulfonyl-cyclopropyl]-6-[(3S)-3-methylmorpholin-4-yl]pyrimidin-2-yl]phenyl]-3-cyclopropylurea | 586 | 2.44 |
| 95c | 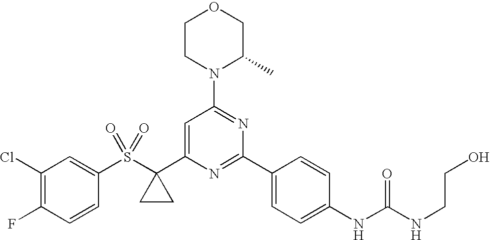 | 1-[4-[4-[1-(3-chloro-4-fluorophenyl)sulfonyl-cyclopropyl]-6-[(3S)-3-methylmorpholin-4-yl]pyrimidin-2-yl]phenyl]-3-(2-hydroxyethyl)urea | 590 | 2.10 |
| 95d | 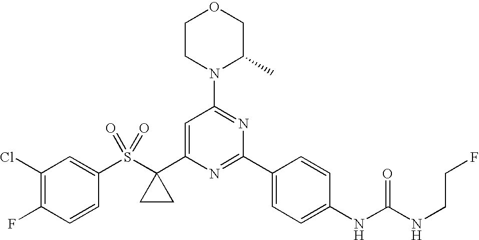 | 1-[4-[4-[1-(3-chloro-4-fluorophenyl)sulfonyl-cyclopropyl]-6-[(3S)-3-methylmorpholin-4-yl]pyrimidin-2-yl]phenyl]-3-(2-fluoroethyl)urea | 592 | 2.41 |
| 95e | 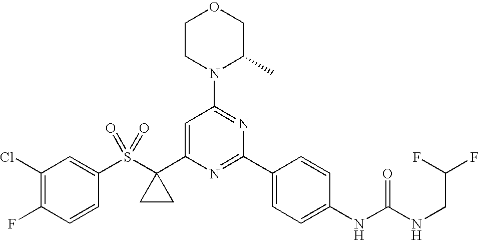 | 1-[4-[4-[1-(3-chloro-4-fluorophenyl)sulfonyl-cyclopropyl]-6-[(3S)-3-methylmorpholin-4-yl]pyrimi-din-2-yl]phenyl]-3-(2,2-difluoroethyl)urea | 610 | 2.54 |
| 95f | 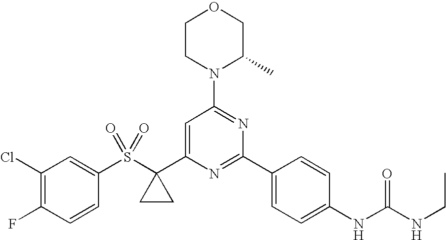 | 3-[4-[4-[1-(3-chloro-4-fluorophenyl)sulfonyl-cyclopropyl]-6-[(3S)-3-methylmorpholin-4-yl]pyrimidin-2-yl]phenyl]-1-ethylurea | 574 | 2.45 |
| 95g | 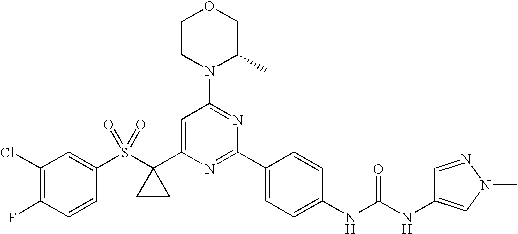 | 1-[4-[4-[1-(3-chloro-4-fluorophenyl)sulfonyl-cyclopropyl]-6-[(3S)-3-methylmorpholin-4-yl]pyrimidin-2-yl]phenyl]-3-(1-methylpyrazol-4-yl)urea | 626 | 2.29 |
| 95h* | 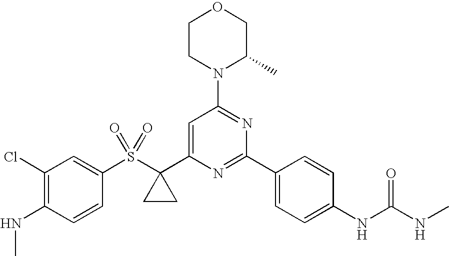 | 1-[4-[4-[1-(3-chloro-4-methylamino-phenyl)sulfonylcyclo-propyl]-6-[(3S)-3-methylmorpho-lin-4-yl]pyrimidin-2-yl]phenyl]-3-methylurea | 571 | 2.11 |
| 95i* | 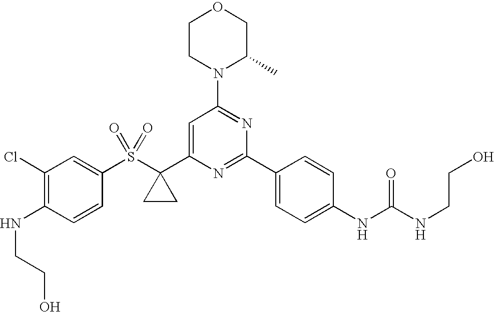 | 1-[4-[4-[1-[3-chloro-4-(2-hydroxyethylamino)phenyl]sulfonylcyclopropyl]-6-[(3S)-3-methylmorpho-lin-4-yl]pyrimidin-2-yl]phenyl]-3-(2-hydroxyethyl)urea | 631 | 1.79 |
| 95j* | 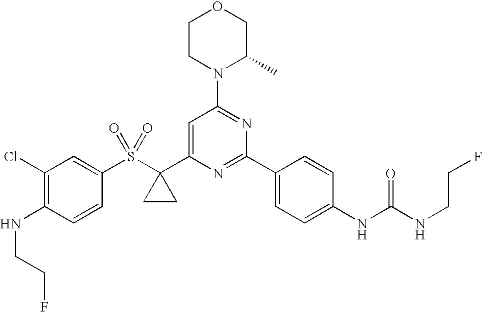 | 1-[4-[4-[1-[3-chloro-4-(2-fluoroethyl-amino)phenyl]sulfonyl-cyclopropyl]-6-[(3S)-3-methylmorpho-lin-4-yl]pyrimidin-2-yl]phenyl]-3-(2-fluoroethyl)urea | 635 | 2.30 |
| 95k* | 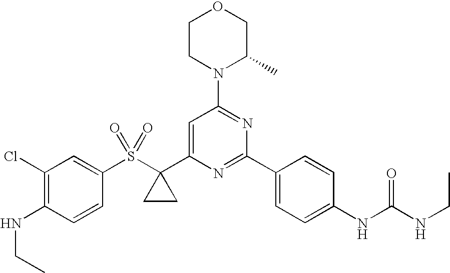 | 1-[4-[4-[1-(3-chloro-4-ethylamino-phenyl)sulfonylcyclopro-pyl]-6-[(3S)-3-methylmorpho-lin-4-yl]pyrimidin-2-yl]phenyl]-3-ethylurea | 599 | 2.41 |
| 95l | 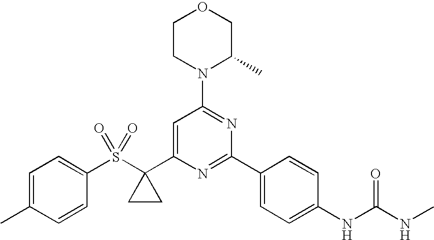 | 3-methyl-1-[4-[4-[(3S)-3-methylmorpholin-4-yl]-6-[1-(4-methylphenyl)sulfonylcyclopropyl]pyrimidin-2-yl]phenyl]urea | 522 | 2.14 |
| 95m | 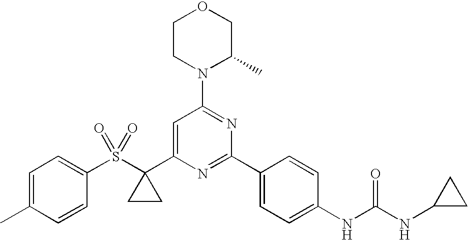 | 3-cyclopropyl-1-[4-[4-[(3S)-3-methylmorpholin-4-yl]-6-[1-(4-methylphenyl)sulfonylcyclopropyl]pyrimidin-2-yl]phenyl]urea | 548 | 2.29 |
| 95n | 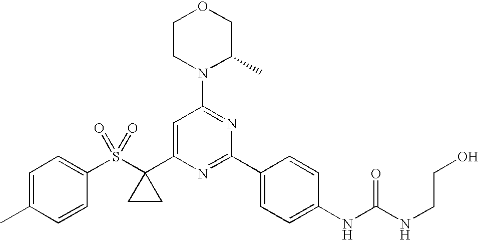 | 3-(2-hydroxyethyl)-1-[4-[4-[(3S)-3-methylmorpholin-4-yl]-6-[1-(4-methylphenyl)sulfonylcyclopropyl]pyrimidin-2-yl]phenyl]urea | 552 | 1.98 |
| 95o | 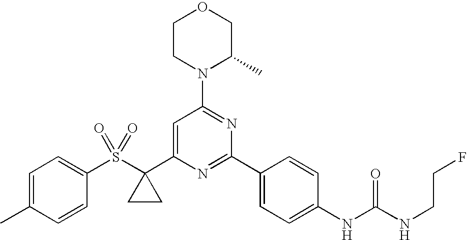 | 3-(2-fluoro-ethyl)-1-[4-[4-[(3S)-3-methylmorpholin-4-yl]-6-[1-(4-methylphenyl)sulfonylcyclopropyl]pyrimidin-2-yl]phenyl]urea | 554 | 2.28 |
| 95p | 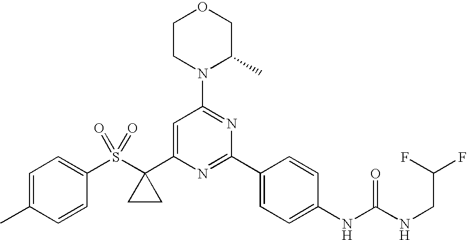 | 3-(2,2-difluoroethyl)-1-[4-[4-[(3S)-3-methylmorpholin-4-yl]-6-[1-(4-methylphenyl)sulfonylcyclopropyl]pyrimidin-2-yl]phenyl]urea | 572 | 2.40 |
| 95q | 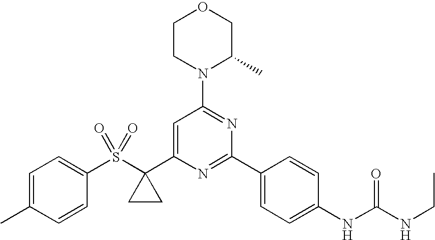 | 1-ethyl-3-[4-[4-[(3S)-3-methylmorpholin-4-yl]-6-[1-(4-methylphenyl)sulfonylcyclopropyl]pyrimidin-2-yl]phenyl]urea | 536 | 2.28 |
| 95r | 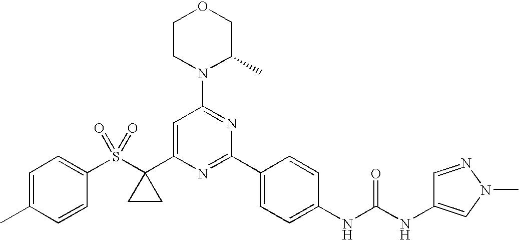 | 1-[4-[4-[(3S)-3-methylmorpholin-4-yl]-6-[1-(4-methylphenyl)sulfonylcyclopropyl]pyrimidin-2-yl]phenyl]-3-(1-methylpyrazol-4-yl)urea | 588 | 2.16 |

*4 equivalents of amine used.

### EXAMPLE 95a

¹H NMR (400.132 MHz, DMSO-d₆) δ 1.20 (3H, d), 1.59-1.64 (2H, m), 1.90-1.98 (2H, m), 2.68 (3H, s), 3.12-3.23 (1H, m), 3.46 (1H, dd), 3.61 (1H, d), 3.75 (1H, d), 3.96 (1H, d), 4.15 (1H, d), 4.48 (1H, s), 6.04 (1H, s), 6.67 (1H, s), 7.39 (2H, d), 7.58-7.68 (1H, m), 7.73-7.83 (3H, m), 7.99 (1H, d), 8.74 (1H, s)

### EXAMPLE 95b

¹H NMR (400.132 MHz, DMSO-d₆) δ 0.37-0.44 (2H, m), 0.60-0.69 (2H, m), 1.20 (3H, d), 1.60-1.64 (2H, m), 1.89-1.98 (2H, m), 2.56-2.60 (1H, m), 3.11-3.23 (1H, m), 3.42-3.52 (1H, m), 3.61 (1H, d), 3.75 (1H, d), 3.95 (1H, d), 4.15 (1H, d), 4.49 (1H, s), 6.39 (1H, s), 6.67 (1H, s), 7.40 (2H, d), 7.57-7.68 (1H, m), 7.73-7.85 (3H, m), 7.92-8.03 (1H, m), 8.54 (1H, s)

### EXAMPLE 95c

¹H NMR (400.132 MHz, DMSO-d₆) δ 1.20 (3H, d), 1.59-1.64 (2H, m), 1.91-1.96 (2H, m), 3.14-3.22 (2H, m), 3.40-3.50 (3H, m), 3.61 (1H, d), 3.75 (1H, d), 3.96 (1H, d), 4.16 (1H, d), 4.48 (1H, s), 4.72 (1H, t), 6.23 (1H, s), 6.67 (1H, s), 7.38 (2H, d), 7.63 (1H, t), 7.75-7.84 (3H, m), 7.98 (1H, d), 8.80 (1H, s)

### EXAMPLE 95d

¹H NMR (400.132 MHz, DMSO-d₆) δ 1.20 (3H, d), 1.59-1.65 (2H, m), 1.88-1.97 (2H, m), 3.36-3.51 (4H, m), 3.62 (1H, d), 3.75 (1H, d), 3.95 (1H, d), 4.12-4.23 (1H, m), 4.41 (1H, t), 4.45-4.51 (1H, m), 4.53 (1H, t), 6.40 (1H, s), 6.68 (1H, s), 7.39 (2H, d), 7.60-7.69 (1H, m), 7.72-7.86 (3H, m), 7.99 (1H, d), 8.81 (1H, s)

### EXAMPLE 95e

¹H NMR (400.132 MHz, DMSO-d₆) δ 1.20 (3H, d), 1.59-1.65 (2H, m), 1.89-1.97 (2H, m), 3.12-3.22 (1H, m), 3.41-3.64 (3H, m), 3.75 (1H, d), 3.96 (1H, d), 4.15 (1H, d), 4.49 (1H, s), 5.87-6.22 (1H, m), 6.49 (1H, t), 6.68 (1H, s), 7.40 (2H, d), 7.63 (1H, t), 7.77-7.86 (3H, m), 7.97-8.00 (1H, m), 8.92 (1H, s)

### EXAMPLE 95f

¹H NMR (400.132 MHz, DMSO-d₆) δ 1.07 (3H, t), 1.20 (3H, d), 1.59-1.65 (2H, m), 1.90-1.98 (2H, m), 3.09-3.21 (3H, m), 3.42-3.52 (1H, m), 3.61 (1H, d), 3.75 (1H, d), 3.96 (1H, d), 4.09-4.21 (1H, m), 4.44-4.53 (1H, m), 6.12 (1H, t), 6.67 (1H, s), 7.39 (2H, d), 7.63 (1H, t), 7.75-7.85 (3H, m), 7.99 (1H, d), 8.66 (1H, s)

### EXAMPLE 95g

¹H NMR (400.132 MHz, DMSO-d₆) δ 1.20 (3H, d), 1.59-1.66 (2H, m), 1.91-1.99 (2H, m), 3.11-3.21 (1H, m), 3.47 (1H, td), 3.62 (1H, d), 3.70-3.83 (4H, m), 3.96 (1H, d), 4.17 (1H, d), 4.49 (1H, s), 6.69 (1H, s), 7.38 (1H, s), 7.44 (2H, d), 7.64 (1H, t), 7.75-7.85 (3H, m), 7.99 (1H, d), 8.34 (1H, s), 8.84 (1H, s)

### EXAMPLE 95h

¹H NMR (400.132 MHz, DMSO-d₆) δ 1.18 (3H, d), 1.53-1.59 (2H, m), 1.77-1.83 (2H, m), 2.63-2.67 (3H, m), 2.82 (3H, d), 3.10-3.19 (1H, m), 3.46 (1H, dd), 3.61 (1H, d), 3.75 (1H, d), 3.95 (1H, d), 4.09 (1H, d), 4.39 (1H, s), 6.00-6.08 (1H, m), 6.47 (1H, t), 6.59 (1H, s), 6.70 (1H, d), 7.41 (2H, d), 7.48-7.57 (2H, m), 7.94 (2H, d), 8.70 (1H, s)

### EXAMPLE 95i

¹H NMR (400.132 MHz, DMSO-d₆) δ 1.18 (3H, d), 1.53-1.60 (2H, m), 1.77-1.83 (2H, m), 3.14-3.19 (2H, m), 3.25-3.32 (2H, m), 3.39-3.50 (3H, m), 3.55-3.65 (3H, m), 3.75 (1H, d), 3.96 (1H, d), 4.10 (1H, d), 4.38 (1H, s), 4.73 (1H, t), 4.82 (1H, t), 6.15 (1H, t), 6.24 (1H, t), 6.59 (1H, s), 6.84 (1H, d), 7.40 (2H, d), 7.48 (2H, d), 7.54 (1H, d), 7.95 (2H, d), 8.76 (1H, s)

### EXAMPLE 95j

¹H NMR (400.132 MHz, DMSO-d₆) δ 1.18 (3H, d), 1.52-1.60 (2H, m), 1.78-1.83 (2H, m), 3.09-3.19 (1H, m), 3.35-3.49 (4H, m), 3.52-3.58 (1H, m), 3.57-3.65 (2H, m), 3.74 (1H, d), 3.95 (1H, d), 4.07 (1H, d), 4.37-4.44 (2H, m), 4.49-4.55 (1H, m), 4.63 (1H, t), 6.38-6.50 (2H, m), 6.58 (1H, s), 6.89 (1H, d), 7.40 (2H, d), 7.48 (2H, d), 7.53 (1H, s), 7.96 (2H, d), 8.76 (1H, s)

### EXAMPLE 95k

¹H NMR (400.132 MHz, DMSO-d₆) δ 1.06 (3H, t), 1.13-1.21 (6H, m), 1.49-1.58 (2H, m), 1.78-1.82 (2H, m), 3.08-3.18 (2H, m), 3.22-3.33 (7H, m), 3.46 (1H, dd), 3.61 (1H, d), 3.74 (1H, d), 3.95 (1H, d), 4.09 (1H, d), 4.39 (1H, s), 6.13 (1H, t), 6.28 (1H, t), 6.59 (1H, s), 6.77 (1H, d), 7.39 (2H, d), 7.46 (2H, d), 7.52 (1H, s), 7.96 (2H, d), 8.61 (1H, s)

### EXAMPLE 95l

¹H NMR (400.132 MHz, DMSO-d₆) δ 1.17 (3H, d), 1.56-1.64 (2H, m), 1.82-1.89 (2H, m), 2.40 (3H, s), 2.68 (3H, s), 3.08-3.17 (1H, m), 3.45 (1H, td), 3.60 (1H, d), 3.74 (1H, d), 3.95 (1H, d), 4.10 (1H, d), 4.38 (1H, s), 6.04 (1H, t), 6.61 (1H, s), 7.33-7.44 (4H, m), 7.66 (2H, d), 7.82 (2H, d), 8.70 (1H, s)

### EXAMPLE 95m

¹H NMR (400.132 MHz, DMSO-d₆) δ 0.42-0.50 (2H, m), 0.66-0.75 (2H, m), 1.23 (3H, d), 1.60-1.69 (2H, m), 1.87-1.98 (2H, m), 2.46 (3H, s), 2.62-2.70 (1H, m), 3.19 (1H, td), 3.46-3.57 (1H, m), 3.66 (1H, d), 3.80 (1H, d), 4.01 (1H, d), 4.16 (1H, d), 4.43 (1H, s), 6.46 (1H, s), 6.67 (1H, s), 7.39-7.49 (4H, m), 7.71 (2H, d), 7.88 (2H, d), 8.56 (1H, s)

### EXAMPLE 95n

¹H NMR (400.132 MHz, DMSO-d₆) δ 1.17 (3H, d), 1.57-1.65 (2H, m), 1.83-1.90 (2H, m), 2.42 (3H, s), 3.13-3.20 (3H, m), 3.40-3.50 (3H, m), 3.60 (1H, d), 3.74 (1H, d), 3.95 (1H, d), 4.09 (1H, d), 4.37 (1H, s), 4.72 (1H, t), 6.22 (1H, t), 6.61 (1H, s), 7.35-7.43 (4H, m), 7.66 (2H, d), 7.82 (2H, d), 8.77 (1H, s)

### EXAMPLE 95o

¹H NMR (400.132 MHz, DMSO-d₆) δ 1.17 (3H, d), 1.56-1.65 (2H, m), 1.81-1.90 (2H, m), 2.40 (3H, s), 3.07-3.22 (1H, m), 3.36-3.50 (3H, m), 3.60 (1H, d), 3.74 (1H, d), 3.96 (1H, d), 4.12 (1H, d), 4.33-4.45 (2H, m), 4.53 (1H, t), 6.41 (1H, t), 6.61 (1H, s), 7.34-7.43 (4H, m), 7.66 (2H, d), 7.83 (2H, d), 8.77 (1H, s)

### EXAMPLE 95p

¹H NMR (400.132 MHz, DMSO-d₆) δ 1.17 (3H, d), 1.56-1.65 (2H, m), 1.82-1.91 (2H, m), 2.40 (3H, s), 3.10-3.18 (1H, m), 3.41-3.65 (4H, m), 3.74 (1H, d), 3.96 (1H, d), 4.09 (1H, d), 4.37 (1H, s), 5.91-6.23 (1H, m), 6.50 (1H, t), 6.62 (1H, s), 7.35-7.44 (4H, m), 7.66 (2H, d), 7.84 (2H, d), 8.89 (1H, s)

### EXAMPLE 95q

¹H NMR (400.132 MHz, DMSO-d₆) δ 1.06 (3H, t), 1.17 (3H, d), 1.52-1.66 (2H, m), 1.85-1.90 (2H, m), 2.40 (3H, s), 3.07-3.20 (3H, m), 3.45 (1H, td), 3.60 (1H, d), 3.74 (1H, d), 3.95 (1H, d), 4.12 (1H, d), 4.37 (1H, s), 6.13 (1H, t), 6.61 (1H, s), 7.35-7.44 (4H, m), 7.66 (2H, d), 7.82 (2H, d), 8.62 (1H, s)

### EXAMPLE 95r

¹H NMR (400.132 MHz, DMSO-d₆) δ 1.18 (3H, d), 1.57-1.66 (2H, m), 1.84-1.90 (2H, m), 2.41 (3H, s), 3.08-3.18 (1H, m), 3.46 (1H, td), 3.61 (1H, d), 3.72-3.83 (4H, m), 3.96 (1H, d), 4.10 (1H, d), 4.37 (1H, s), 6.62 (1H, s), 7.35-7.49 (5H, m), 7.66 (2H, d), 7.76 (1H, s), 7.86 (2H, d), 8.35 (1H, s), 8.80 (1H, s)

The preparation of phenyl N-[4-[4-[1-(3-chloro-4-fluorophenyl)sulfonylcyclopropyl]-6-[(3S)-3-methylmorpholin-4-yl]pyrimidin-2-yl]phenyl]carbamate is described below.

### Phenyl N-[4-[4-[1-(3-chloro-4-fluorophenyl)sulfonylcyclopropyl]-6-[(3S)-3-methylmorpholin-4-yl]pyrimidin-2-yl]phenyl]carbamate

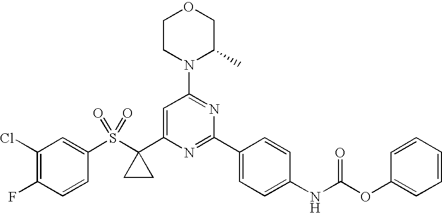

Sodium hydrogen carbonate (0.501 g, 5.96 mmol) was added to 4-[4-[1-(3-chloro-4-fluorophenyl)sulfonylcyclopropyl]-6-[(3S)-3-methylmorpholin-4-yl]pyrimidin-2-yl]aniline (2 g, 3.98 mmol) in dioxane (30 mL) at 5° C. under nitrogen. Phenyl chloroformate (0.749 mL, 5.96 mmol) was then added and the resulting mixture stirred at RT for 2 hours. The reaction mixture was diluted with DCM (50 mL) and washed with water (75 mL). The organic layer was dried over Na₂SO₄, filtered and evaporated to afford crude product. The crude product was triturated with a mixture of diethyl ether and isohexane to give the desired material as a cream solid (1.45 g).

NMR Spectrum: ¹H NMR (400.132 MHz, DMSO-d₆) δ 1.21 (3H, d), 1.59-1.66 (2H, m), 1.91-1.96 (2H, m), 3.12-3.24 (1H, m), 3.40-3.52 (1H, m), 3.62 (1H, d), 3.75 (1H, d), 3.96 (1H, d), 4.18 (1H, d), 4.50 (1H, s), 6.74 (1H, s), 7.23-7.32 (3H, m), 7.41-7.49 (2H, m), 7.54 (2H, d), 7.58-7.68 (1H, m), 7.79-7.83 (1H, m), 7.88 (2H, d), 7.96-8.02 (1H, m), 10.40 (1H, s)

LCMS Spectrum: m/z (ESI+)(M+H)+=622; HPLC tR=3.21 min.

### 4-[4-[1-(3-Chloro-4-fluorophenyl)sulfonylcyclopropyl]-6-[(3S)-3-methylmorpholin-4-yl]pyrimidin-2-yl]aniline

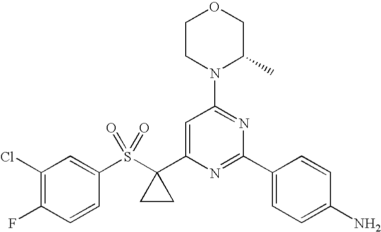

Bis(triphenylphosphine)palladium(II) chloride (0.242 g, 0.35 mmol) was added to 2-chloro-4-[1-(3-chloro-4-fluorophenyl)sulfonylcyclopropyl]-6-[(3S)-3-methylmorpholin-4-yl]pyrimidine (2.3 g, 5.15 mmol), 4-(4,4,5,5-tetramethyl-1,3,2-dioxaborolan-2-yl)aniline (1.411 g, 6.44 mmol) and 2M aqueous sodium carbonate solution (3 mL, 6.00 mmol) in a solvent mixture of DMF (5 mL), DME (8 mL), water (2 mL) and ethanol (1.5 mL) at RT. The resulting mixture was stirred at 90° C., under an inert atmosphere for 5 hours then left at RT for 16 hours. The reaction mixture was diluted with ethyl acetate (200 mL), and washed sequentially with water (2×100 mL). The organic layer was dried over Na₂SO₄, filtered and evaporated to afford crude product. The crude product was purified by flash silica chromatography, elution gradient 20 to 80% ethyl acetate in isohexane, to give the desired material as a yellow gum (2.0 g).

NMR Spectrum: ¹H NMR (400.132 MHz, DMSO-d₆) δ 1.18 (3H, d), 1.57-1.61 (2H, m), 1.87-1.96 (2H, m), 3.14 (1H, td), 3.41-3.50 (1H, m), 3.60 (1H, d), 3.74 (1H, d), 3.95 (1H, d), 4.13 (1H, d), 4.44 (1H, s), 5.52 (1H, d), 6.49 (2H, d), 6.57 (1H, s), 7.57-7.68 (3H, m), 7.75-7.85 (1H, m), 7.99 (1H, d)

LCMS Spectrum: m/z (ESI+)(M+H)+=503; HPLC tR=2.66 min.

### 2-Chloro-4-[1-(3-chloro-4-fluorophenyl)sulfonylcyclopropyl]-6-[(3S)-3-methylmorpholin-4-yl]pyrimidine

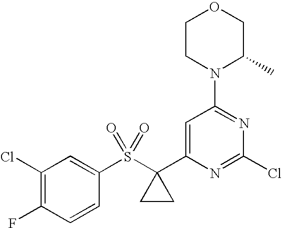

Aqueous sodium hydroxide solution (20 mL, 142.76 mmol) was added to 2-chloro-4-[(3-chloro-4-fluorophenyl)sulfonylmethyl]-6-[(3S)-3-methylmorpholin-4-yl]pyrimidine (2.5 g, 5.95 mmol), 1,2-dibromoethane (2.56 mL, 29.74 mmol) and tetrabutylammonium bromide (0.192 g, 0.59 mmol) in toluene (100 mL) at RT under a nitrogen atmosphere and the mixture stirred for 3 hours. The reaction mixture was diluted with ethyl acetate (100 mL) and washed with water (50 mL). The organic layer was dried over MgSO₄, filtered and evaporated to afford crude product. The crude product was purified by flash silica chromatography, elution gradient 10 to 100% ethyl acetate in isohexane, to give the desired material as a colourless gum (2.3 g).

NMR Spectrum: ¹H NMR (400.132 MHz, DMSO-d₆) δ 7.98 (1H, d), 1.18 (3H, d), 1.54-1.61 (2H, m), 1.84-1.91 (2H, m), 3.15 (1H, td), 3.36-3.45 (1H, m), 3.55 (1H, d), 3.70 (1H, d), 3.90 (1H, d), 4.08 (1H, s), 4.31 (1H, s), 6.74 (1H, s), 7.62-7.71 (1H, m), 7.76-7.83 (1H, m)

LCMS Spectrum: m/z (ESI+)(M+H)+=446; HPLC tR=2.56 min.

### 2-Chloro-4-[(3-chloro-4-fluorophenyl)sulfonylmethyl]-6-[(3S)-3-methylmorpholin-4-yl]pyrimidine

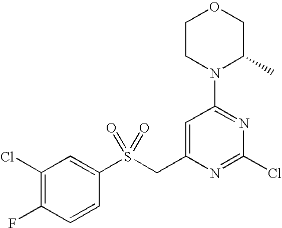

Sodium 3-chloro-4-fluorobenzenesulfinate (3.52 g, 16.26 mmol) was added to 2-chloro-4-(iodomethyl)-6-[(3S)-3-methylmorpholin-4-yl]pyrimidine (5 g, 14.14 mmol) in acetonitrile (150 mL) at RT under a nitrogen atmosphere. The resulting mixture was stirred at 80° C. for 3 hours. The reaction mixture was diluted with ethyl acetate (150 mL) and washed with water (100 mL), The organic layer was dried over MgSO₄, filtered and evaporated to afford crude product. The crude product was purified by flash silica chromatography, elution gradient 0 to 70% ethyl acetate in isohexane, to give a colourless oil which solidified on standing. The crude solid was triturated with a mixture of diethyl ether and isohexane to give the desired material as a white solid (4.0 g).

NMR Spectrum: ¹H NMR (400.132 MHz, DMSO-d₆) δ 1.18 (3H, d), 3.18 (1H, dd), 3.28 (1H, d), 3.43 (1H, td), 3.58 (1H, d), 3.72 (1H, d), 3.94 (1H, d), 4.21 (1H, s), 4.73 (2H, s), 6.78 (1H, s), 7.66-7.74 (1H, m), 7.78-7.87 (1H, m), 8.02 (1H, d)

LCMS Spectrum: m/z (ESI+)(M+H)+=420; HPLC tR=2.38 min.

The preparation of 2-chloro-4-(iodomethyl)-6-[(3S)-3-methylmorpholin-4-yl]pyrimidine was described earlier.

### Sodium 3-chloro-4-fluorobenzenesulfinate

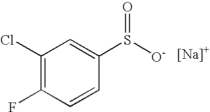

A solution of sodium sulfite (8.25 g, 65.49 mmol) in water (75 mL) was stirred at RT for 10 minutes. Sodium bicarbonate (11.0 g, 130.97 mmol) was added to the stirred solution and the resulting solution stirred at 50° C. for 1 hour. 3-Chloro-4-fluorobenzene-1-sulfonyl chloride (15 g, 65.49 mmol) was added portionwise to the solution and was stirred at 50° C. for 18 hours. The reaction mixture was evaporated to dryness and redissolved in methanol (300 mL). The suspension was allowed to stir at RT for 20 minutes then filtered and the filtrate evaporated to afford the desired material as a white solid, which was air dried overnight under vacuum and used without further purification (16.5 g).

NMR Spectrum: ¹H NMR (400.132 MHz, DMSO-d₆) δ 7.34 (1H, t), 7.40-7.46 (1H, m), 7.58 (1H, d)

The preparation of phenyl N-[4-[4-[(3S)-3-methylmorpholin-4-yl]-6-[1-(4-methylphenyl)sulfonylcyclopropyl]pyrimidin-2-yl]phenyl]carbamate is described below.

### Phenyl N-[4-[4-[(3S)-3-methylmorpholin-4-yl]-6-[1-(4-methylphenyl)sulfonylcyclopropyl]pyrimidin-2-yl]phenyl]carbamate

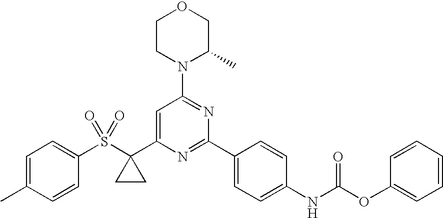

Sodium hydrogen carbonate (0.570 g, 6.78 mmol) was added to 4-[4-[(3S)-3-methylmorpholin-4-yl]-6-[1-(4-methylphenyl)sulfonylcyclopropyl]pyrimidin-2-yl]aniline (2.1 g, 4.52 mmol) in dioxane (6 mL) at 5° C. under a nitrogen atmosphere. Phenyl chloroformate (0.852 mL, 6.78 mmol) was then added and the resulting mixture stirred at RT for 2 hours. The reaction mixture was diluted with DCM (50 mL) and washed with water (75 mL). The organic layer was dried over Na₂SO₄, filtered and evaporated to afford crude product. The crude gum was triturated with a mixture of diethyl ether and isohexane to give the desired material as a cream solid (1.52 g).

NMR Spectrum: ¹H NMR (400.132 MHz, DMSO-d₆) δ 1.17 (3H, d), 1.58-1.64 (2H, m), 1.85-1.91 (2H, m), 2.40 (3H, s), 3.09-3.23 (1H, m), 3.41-3.51 (1H, m), 3.60 (1H, d), 3.75 (1H, d), 3.96 (1H, d), 4.12 (1H, d), 4.39 (1H, s), 6.65 (1H, s), 7.21-7.31 (3H, m), 7.36-7.41 (2H, m), 7.42-7.48 (2H, m), 7.50-7.56 (2H, m), 7.63-7.71 (2H, m), 7.89 (2H, d), 10.38 (1H, s)

LCMS Spectrum: m/z (ESI+)(M+H)+=535; HPLC tR=3.1 min.

### 4-[4-[(3S)-3-Methylmorpholin-4-yl]-6-[1-(4-methylphenyl)sulfonylcyclopropyl]pyrimidin-2-yl]aniline

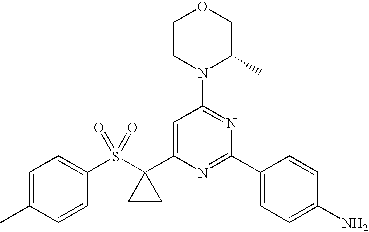

Bis(triphenylphosphine)palladium(II) chloride (0.225 g, 0.32 mmol) was added to 2-chloro-4-[(3S)-3-methylmorpholin-4-yl]-6-[1-(4-methylphenyl)sulfonylcyclopropyl]pyrimidine (1.95 g, 4.78 mmol), 4-(4,4,5,5-tetramethyl-1,3,2-dioxaborolan-2-yl)aniline (1.362 g, 6.21 mmol) and 2M aqueous sodium carbonate solution (3 mL, 6.00 mmol) in a solvent mixture of DMF (5 mL), DME (8 mL), water (2 mL) and ethanol (1.5 mL) at RT. The resulting mixture was stirred at 90° C. for 5 hours under an inert atmosphere, then left at RT for 16 hours. The reaction mixture was diluted with ethyl acetate (200 mL) and washed with water (2×100 mL). The organic layer was dried over Na₂SO₄, filtered and evaporated to afford crude product. The crude product was purified by flash silica chromatography, elution gradient 20 to 80% ethyl acetate in isohexane, to give crude material which was further purified by ion exchange chromatography using an SCX column, eluting with 7M ammonia in methanol, to give the desired material as a white solid (2.1 g).

NMR Spectrum: ¹H NMR (400.132 MHz, DMSO-d₆) δ 1.15 (3H, d), 1.54-1.63 (2H, m), 1.81-1.89 (2H, m), 1.99 (3H, s), 3.11 (1H, td), 3.37-3.49 (1H, m), 3.59 (1H, d), 3.73 (1H, d), 3.94 (1H, d), 4.06 (1H, s), 4.32 (1H, s), 5.50 (1H, s), 6.50 (3H, d), 7.38 (2H, d), 7.62-7.72 (4H, m)

LCMS Spectrum: m/z (ESI+)(M+H)+=465; HPLC tR=2.48 min.

### 2-Chloro-4-[(3S)-3-methylmorpholin-4-yl]-6-[1-(4-methylphenyl)sulfonylcyclopropyl]pyrimidine

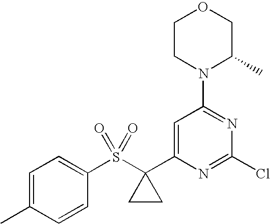

Aqueous sodium hydroxide solution (20 mL, 125.69 mmol) was added to 2-chloro-4-[(3S)-3-methylmorpholin-4-yl]-6-[(4-methylphenyl)sulfonylmethyl]pyrimidine (2 g, 5.24 mmol), 1,2-dibromoethane (2.257 mL, 26.19 mmol) and tetrabutylammonium bromide (0.169 g, 0.52 mmol) in DCM (100 mL) at RT under a nitrogen atmosphere then stirred for 3 hours. The reaction mixture was diluted with ethyl acetate (100 mL), and washed with water (50 mL). The organic layer was dried over MgSO₄, filtered and evaporated to afford crude product. The crude product was purified by flash silica chromatography, elution gradient 10 to 60% ethyl acetate in isohexane, to give the desired material as a colourless gum (1.9 g).

NMR Spectrum: ¹H NMR (400.132 MHz, DMSO-d₆) δ 1.13 (3H, d), 1.52-1.56 (2H, m), 1.79-1.84 (2H, m), 1.99 (3H, s), 3.12 (1H, td), 3.35-3.47 (1H, m), 3.54 (1H, d), 3.71 (1H, d), 3.84-3.95 (2H, m), 4.17 (1H, s), 6.63 (1H, s), 7.41 (2H, d), 7.63 (2H, d)

LCMS Spectrum: m/z (ESI+)(M+H)+=408; HPLC tR=2.35 min.

### 2-Chloro-4-[(3S)-3-methylmorpholin-4-yl]-6-[(4-methylphenyl)sulfonylmethyl]pyrimidine

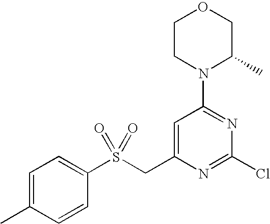

Sodium 4-methylbenzenesulfinate (2.9 g, 16.28 mmol) was added to 2-chloro-4-(iodomethyl)-6-[(3S)-3-methylmorpholin-4-yl]pyrimidine (5 g, 14.14 mmol), in acetonitrile (150 mL) at RT under a nitrogen atmosphere. The resulting mixture was stirred at 80° C. for 3 hours. The reaction mixture was diluted with ethyl acetate (150 mL) and washed with water (100 mL). The organic layer was dried over MgSO₄, filtered and evaporated to afford crude product. The crude product was purified by flash silica chromatography, elution gradient 0 to 40% ethyl acetate in isohexane, to give the desired material as a beige solid (3.10 g).

NMR Spectrum: ¹H NMR (400.132 MHz, DMSO-d₆) δ 1.15 (3H, d), 2.41 (3H, s), 3.14 (1H, td), 3.38-3.46 (1H, m), 3.56 (1H, d), 3.71 (1H, d), 3.80-3.96 (2H, m), 4.13 (1H, s), 4.60 (2H, s), 6.60 (1H, s), 7.43 (2H, d), 7.66 (2H, d)

LCMS Spectrum: m/z (ESI+)(M+H)+=382; HPLC tR=2.16 min.

The preparation of 2-chloro-4-(iodomethyl)-6-[(3S)-3-methylmorpholin-4-yl]pyrimidine was described earlier.

### Sodium 4-methylbenzenesulfinate

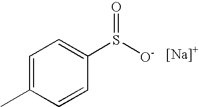

A solution of sodium sulfite (9.92 g, 78.68 mmol) in water (75 mL) was stirred at RT for 10 minutes. Sodium bicarbonate (13.22 g, 157.36 mmol) was added to the stirred solution. The resulting solution was stirred at 50° C. for 1 hour. 4-Methylbenzene-1-sulfonyl chloride (15 g, 78.68 mmol) was added portionwise to the solution and was stirred at 50° C. for 18 hours. The reaction mixture was evaporated to dryness and redissolved in methanol (400 mL). The suspension was allowed to stir at RT for 20 minutes then filtered and the filtrate evaporated to afford the desired material as a white solid, which was air dried overnight under vacuum and used without further purification (17.3 g).

### EXAMPLE 96

The following samples were prepared by heating a mixture of the carbamate (1 equivalent), triethylamine (4 equivalents) and the amine (4 equivalents) in NMP (2 mL) at RT for between 2-16 hours. The compounds were purified by preparative HPLC.

The following compounds were prepared in an analogous fashion from phenyl N-[4-[4-[1-[3-(difluoromethoxy)propylsulfonyl]cyclopropyl]-6-[(3S)-3-methylmorpholin-4-yl]pyrimidin-2-yl]phenyl]carbamate (contaminated with phenyl N-[4-[4-[1-[3-(difluoromethoxy)propylsulfonyl]propyl]-6-[(3S)-3-methylmorpholin-4-yl]pyrimidin-2-yl]phenyl]carbamate) and the appropriate amine.

| | | | | Retention |
|---|---|---|---|---|
| Ex- | | | LCMS | time |
| ample | Structure | NAME | MH+ | (min) |
| 96a | 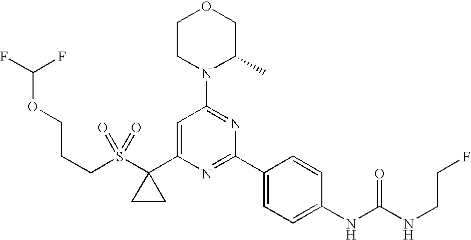 | 1-[4-[4-[1-[3-(difluoromethoxy)propylsulfonyl]cyclopropyl]-6-[(3S)-3-methylmorpholin-4-yl]pyrimidin-2-yl]phenyl]-3-(2-fluoroethyl)urea | 572 | 2.28 |
| 96b. | 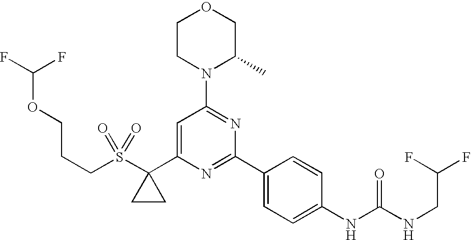 | 3-(2,2-difluoroethyl)-1-[4-[4-[1-[3-(difluoromethoxy)propylsulfonyl]cycloproyl]-6-[(3S)-3-methylmorpholin-4-yl]pyrimidin-2-yl]phenyl]urea | 590 | 2.32 |
| 96c | 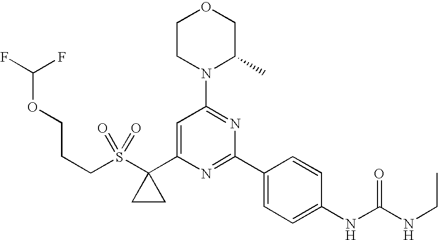 | 1-[4-[4-[1-[3-(difluoromethoxy)propylsulfonyl]cyclopropyl]-6-[(3S)-3-methylmorpholin-4-yl]pyrimidin-2-yl]phenyl]-3-ethylurea | 554 | 2.21 |
| 96d | 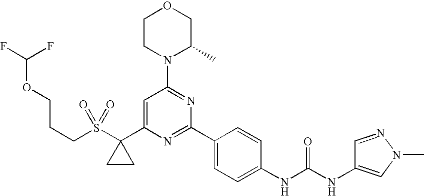 | 1-[4-[4-[1-[3-(difluoromethoxy)propylsulfonyl]cyclopropyl]-6-[(3S)-3-methylmorpholin-4-yl]pyrimidin-2-yl]phenyl]-3-(1-methylpyrazol-4-yl)urea | 606 | 2.09 |
| 96e | 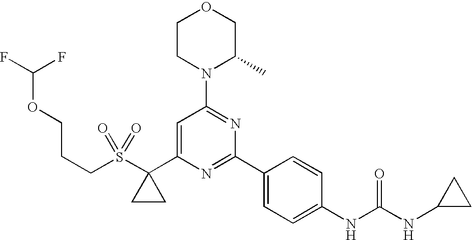 | 3-cyclopropyl-1-[4-[4-[1-[3-(difluoromethoxy)propylsulfonyl]cyclopropyl]-6-[(3S)-3-methylmorpholin-4-yl]pyrimidin-2-yl]phenyl]urea | none | |

### EXAMPLE 96a

¹H NMR (400.132 MHz, DMSO-d₆) δ 1.23 (3H, d), 1.54-1.59 (2H, m), 1.62-1.69 (2H, m), 2.09-2.18 (2H, m), 3.17-3.25 (1H, m), 3.35-3.42 (1H, m), 3.43-3.52 (2H, m), 3.56-3.66 (3H, m), 3.78 (1H, d), 3.94-4.02 (3H, m), 4.21 (1H, d), 4.42 (1H, t), 4.52-4.60 (2H, m), 6.43 (1H, t), 6.47-6.89 (1H, m), 6.77 (1H, s), 7.49 (2H, d), 8.20 (2H, d), 8.81 (1H, s)

### EXAMPLE 96b

¹H NMR (400.132 MHz, DMSO-d₆) δ 1.23 (3H, d), 1.53-1.60 (2H, m), 1.62-1.69 (2H, m), 2.08-2.17 (2H, m), 3.16-3.25 (1H, m), 3.43-3.66 (6H, m), 3.76 (1H, d), 3.94-4.00 (3H, m), 4.22 (1H, d), 4.56 (1H, s), 5.91-6.23 (1H, m), 6.43-6.88 (1H, m), 6.52 (1H, t), 6.77 (1H, s), 7.50 (2H, d), 8.21 (2H, d), 8.93 (1H, s)

### EXAMPLE 96c

¹H NMR (400.132 MHz, DMSO-d₆) δ 1.08 (3H, t), 1.23 (3H, d), 1.54-1.60 (2H, m), 1.63-1.69 (2H, m), 2.09-2.18 (2H, m), 3.07-3.25 (3H, m), 3.48 (1H, td), 3.55-3.66 (3H, m), 3.76 (1H, d), 3.93-4.01 (3H, m), 4.21 (1H, d), 4.56 (1H, s), 6.15 (1H, t), 6.42-6.88 (1H, m), 6.76 (1H, s), 7.49 (2H, d), 8.18 (2H, d), 8.66 (1H, s)

### EXAMPLE 96d

¹H NMR (400.132 MHz, DMSO-d₆) δ 1.24 (3H, d), 1.55-1.60 (2H, m), 1.63-1.69 (2H, m), 2.09-2.18 (2H, m), 3.18-3.26 (1H, m), 3.44-3.53 (1H, m), 3.55-3.67 (3H, m), 3.72-3.80 (4H, m), 3.93-4.03 (3H, m), 4.22 (1H, d), 4.57 (1H, s), 6.43-6.86 (1H, m), 6.78 (1H, s), 7.38 (1H, s), 7.54 (2H, d), 7.77 (1H, s), 8.22 (2H, d), 8.37 (1H, s), 8.85 (1H, s)

### EXAMPLE 96e

¹H NMR (400.132 MHz, DMSO-d₆) δ 0.38-0.40 (2H,m), 0.61-0.64 (2H,m), 1.20-1.22 (3H,d), 1.51-1.53 (2H,m), 1.61-1.64 (2H,m), 2.18-2.24 (2H,m), 2.52-2.58 (1H,m), 3.18-3.20 (1H,dd), 3.55-3.68 (3H,m), 3.72-3.78 (1H,m), 3.90-3.95 (3H,m), 4.20 (1H,s), 4.55 (1H,s), 6.40 (1H,s), 6.60 (1H,s), 7.45 (2H,d), 8.18 (2H,d), 8.54 (1H,s)

The preparation of phenyl N-[4-[4-[1-[3-(difluoromethoxy)propylsulfonyl]cyclopropyl]-6-[(3S)-3-methylmorpholin-4-yl]pyrimidin-2-yl]phenyl]carbamate is described below.

### Phenyl N-[4-[4-[1-[3-(difluoromethoxy)propylsulfonyl]cyclopropyl]-6-[(3s)-3-methylmorpholin-4-yl]pyrimidin-2-yl]phenyl]carbamate

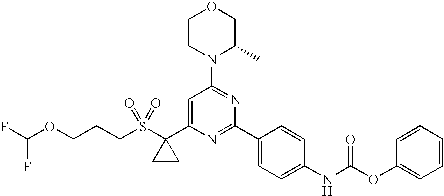

Sodium hydrogen carbonate (65.3 mg, 0.78 mmol) was added to 4-[4-[1-[3-(difluoromethoxy)propylsulfonyl]cyclopropyl]-6-[(3S)-3-methylmorpholin-4-yl]pyrimidin-2-yl]aniline (contaminated with 4-[4-[1-[3-(difluoromethoxy)propylsulfonyl]propyl]-6-[(3S)-3-methylmorpholin-4-yl]pyrimidin-2-yl]aniline) (250 mg, 0.52 mmol) in dioxane (6 mL) at 5° C. under a nitrogen atmosphere. Phenyl chloroformate (0.098 mL, 0.78 mmol) was added and the resulting mixture stirred at RT for 2 hours. The reaction mixture was diluted with DCM (50 mL), the organics dried over Na₂SO₄, filtered and evaporated to afford crude product. The crude material was triturated with a mixture of diethyl ether and isohexane to give the desired material (contaminated with phenyl N-[4-[4-[1-[3-(difluoromethoxy)propylsulfonyl]propyl]-6-[(3S)-3-methylmorpholin-4-yl]pyrimidin-2-yl]phenyl]carbamate) as a cream solid (200 mg). The material was used without further purification.

NMR Spectrum: none

LCMS Spectrum: m/z (ESI+)(M+H)+=604; HPLC tR=2.97 min.

### 4-[4-[1-[3-(Difluoromethoxy)propylsulfonyl]cyclopropyl]-6-[(3S)-3-methylmorpholin-4-yl]pyrimidin-2-yl]aniline

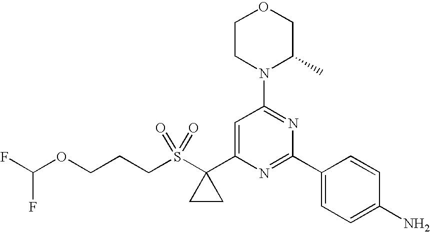

5% Palladium on charcoal (400 mg, 3.6 mmol) was added to 4-[1-[3-(difluoromethoxy)propylsulfonyl]cyclopropyl]-6-[(3S)-3-methylmorpholin-4-yl]-2-(4-nitrophenyl)pyrimidine (2 g, 3.90 mmol) in ethyl acetate (200 mL) and methanol (30 mL) at RT. The flask was evacuated and the atmosphere replaced with first nitrogen and then hydrogen and the mixture left to stir under hydrogen at RT for 36 hours. The crude product was purified by flash silica chromatography, elution gradient 30 to 90% ethyl acetate in isohexane, to give a white solid (1.7 g) which appears to be the desired material (˜30%) contaminated with 4-[4-[1-[3-(difluoromethoxy)propylsulfonyl]propyl]-6-[(3S)-3-methylmorpholin-4-yl]pyrimidin-2-yl]aniline (60%). The material was used without further purification.

NMR Spectrum: none

LCMS Spectrum: m/z (ESI+)(M+H)+=483; HPLC tR=1.76 min.

LCMS Spectrum: (4-[4-[1-[3-(difluoromethoxy)propylsulfonyl]propyl]-6-[(3S)-3-methylmorpholin-4-yl]pyrimidin-2-yl]aniline) m/z (ESI+)(M+H)+=485; HPLC tR=2.0 min.

### 4-[1-[3-(Difluoromethoxy)propylsulfonyl]cyclopropyl]-6-[(3S)-3-methylmorpholin-4-yl]-2-(4-nitrophenyl)pyrimidine

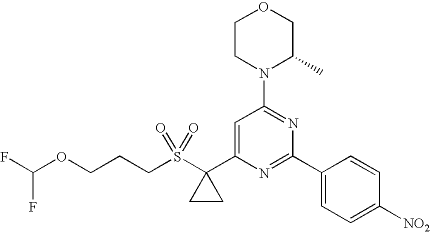

A solution of 2,2-difluoro-2-(fluorosulfonyl)acetic acid (4.56 mL, 44.11 mmol) in acetonitrile (25 mL) was added dropwise over 1.5 hours to a stirred solution of 3-[1-[6-[(3S)-3-methylmorpholin-4-yl]-2-(4-nitrophenyl)pyrimidin-4-yl]cyclopropyl]sulfonylpropan-1-ol (3.4g, 7.35 mmol) and copper(I) iodide (280 mg, 1.47 mmol) in acetonitrile (100 mL) at 55° C. under a nitrogen atmosphere. The mixture was stirred at 55° C. for a further 1 hour then diluted with ethyl acetate (300 mL) and washed with water (100 mL). The organic layer was dried over Na₂SO₄, filtered and evaporated to afford crude product. The crude product was purified by flash silica chromatography, elution gradient 10 to 50% ethyl acetate in isohexane, to give the desired material as a yellow solid (2.0 g).

NMR Spectrum: ¹H NMR (400.132 MHz, DMSO-d₆) δ 1.26 (3H, d), 1.59-1.64 (2H, m), 1.67-1.72 (2H, m), 2.11-2.17 (2H, m), 3.22-3.28 (1H, m), 3.46-3.59 (3H, m), 3.65 (1H, d), 3.78 (1H, d), 3.93-4.01 (3H, m), 4.25 (1H, s), 4.61 (1H, s), 6.96 (1H, s), 8.33 (2H, d), 8.55 (2H, d)

LCMS Spectrum: m/z (ESI+)(M+H)+=513; HPLC tR=2.87 min.

### 3-[1-[6-[(3S)-3-Methylmorpholin-4-yl]-2-(4-nitrophenyl)pyrimidin-4-yl]cyclopropyl]sulfonylpropan-1-ol

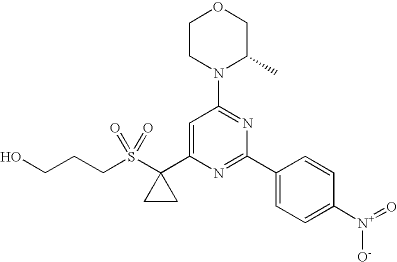

Bis(triphenylphosphine)palladium(II) chloride (0.636 g, 0.91 mmol) was added to 3-[1-[2-chloro-6-[(3S)-3-methylmorpholin-4-yl]pyrimidin-4-yl]cyclopropyl]sulfonylpropoxy-tri(propan-2-yl)silane (7.2 g, 13.53 mmol), 2M aqueous sodium carbonate solution (15 mL, 30.00 mmol) and 4-(4,4,5,5-tetramethyl-1,3,2-dioxaborolan-2-yl)nitrobenzene (6.74 g, 27.06 mmol) in DME (100 mL) and water (5 mL) at RT. The mixture was stirred at 90° C. for 16 hours under a nitrogen atmosphere then allowed to cool. The reaction mixture was diluted with ethyl acetate (400 mL), and washed sequentially with water (100 mL) then additional water (200 mL). The organic layer was dried over Na₂SO₄, filtered and evaporated to afford crude product. The crude product was dissolved in DCM then tetrabutylammonium fluoride (67.6 mL, 67.64 mmol) added and left to stir for 1 hour. A saturated aqueous solution of ammonium chloride was added, the organics separated and dried over Na₂SO₄, filtered and evaporated. The crude product was purified by flash silica chromatography, elution gradient 10 to 100% ethyl acetate in isohexane, to give crude material which was further purified by ion exchange chromatography using an SCX column, eluting with 7M ammonia in methanol, to give the desired material as a brown solid (3.60 g).

NMR Spectrum: ¹H NMR (400.132 MHz, DMSO-d₆) δ 1.26 (3H, d), 1.56-1.63 (2H, m), 1.66-1.70 (2H, m), 1.91-1.98 (2H, m), 3.22-3.29 (3H, m), 3.47-3.57 (3H, m), 3.65 (1H, d), 3.78 (1H, d), 3.99 (1H, dd), 4.25 (1H, d), 4.60 (1H, s), 4.72 (1H, t), 6.96 (1H, s), 8.33 (2H, d), 8.58 (2H, d)

LCMS Spectrum: m/z (ESI+)(M+H)+=463; HPLC tR=2.37 min.

The preparation of 3-[1-[2-chloro-6-[(3S)-3-methylmorpholin-4-yl]pyrimidin-4-yl]cyclopropyl]sulfonylpropoxy-tri(propan-2-yl)silane was described earlier.

### EXAMPLE 97

### 3-Methyl-1-[4-[4-[1-(2-methylaminoethylsulfonyl)cyclopropyl]-6-morpholin-4-ylpyrimidin-2-yl]phenyl]urea

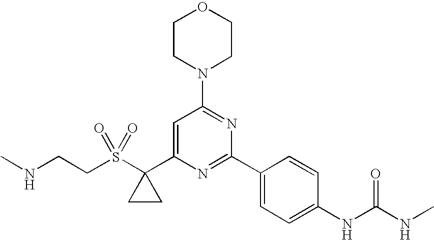

Methylamine (2 M in THF, 4 equivalents) was added to phenyl N-[4-[4-[1-[2-(difluoromethoxy)ethylsulfonyl]cyclopropyl]-6-morpholin-4-ylpyrimidin-2-yl]phenyl]carbamate in NMP and resulting solution stirred at RT for 30 minutes then purified by preparative HPLC to give the desired material (0.012 g)

NMR Spectrum: ¹H NMR (400.132 MHz, DMSO-d₆) δ 1.52-1.57 (2H, m), 1.62-1.66 (2H, m), 2.67 (3H, d), 2.92-3.01 (2H, m), 3.31 (3H, s), 3.55-3.64 (2H, m), 3.72 (8H, s), 6.06 (1H, t), 6.81 (1H, s), 7.50 (2H, d), 8.21 (2H, d), 8.75 (1H, s)

LCMS Spectrum: m/z (ESI+) (M+H)+=475; HPLC tR=1.65 min.

The preparation of phenyl N-[4-[4-[1-[2-(difluoromethoxy)ethylsulfonyl]cyclopropyl]-6-morpholin-4-ylpyrimidin-2-yl]phenyl]carbamate is described below.

### Phenyl N-[4-[4-[1-[2-(difluoromethoxy)ethylsulfonyl]cyclopropyl]-6-morpholin-4-ylpyrimidin-2-yl]phenyl]carbamate

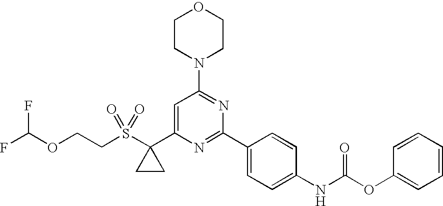

Sodium hydrogen carbonate (1.5 equivalents) was added to 4-[4-[1-[2-(difluoromethoxy)ethylsulfonyl]cyclopropyl]-6-morpholin-4-ylpyrimidin-2-yl]aniline (1 equivalent) in dioxane at 5° C. under nitrogen. Phenyl chloroformate (1.5 equivalents) was then added. The resulting mixture was stirred at RT for 2 hours. The reaction mixture was diluted with DCM, the organics dried over Na₂SO₄, filtered and evaporated to afford crude product. The crude gum was triturated with a mixture of ethyl acetate and isohexane to give the desired material as a cream solid.

NMR Spectrum: none

LCMS Spectrum: m/z (ESI+) (M+H)+=575; HPLC tR=2.8 min.

### 4-[4-[1-[2-(Difluoromethoxy)ethylsulfonyl]cyclopropyl]-6-morpholin-4-ylpyrimidin-2-yl]aniline

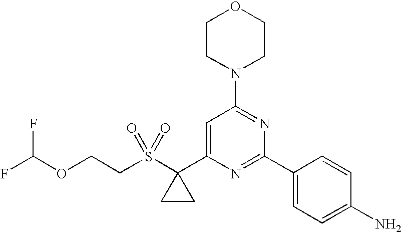

Palladium, 5% On Charcoal (6.59 mg, 0.06 mmol) was added to 4-[1-[2-(difluoromethoxy)ethylsulfonyl]cyclopropyl]-6-morpholin-4-yl-2-(4-nitrophenyl)pyrimidine (150 mg, 0.31 mmol), in ethyl acetate (20 mL) and methanol (3 mL) at RT and left to stir under an atmosphere of hydrogen for 24 hours. The mixture was filtered through celite® and the filtrate purified by flash silica chromatography, elution gradient 10 to 70% ethyl acetate in isohexane, to give the desired material as a yellow gum (90 mg).

NMR Spectrum: none

LCMS Spectrum: m/z (ESI+) (M+H)+=455; HPLC tR=2.2 min.

### 4-[1-[2-(Difluoromethoxy)ethylsulfonyl]cyclopropyl]-6-morpholin-4-yl-2-(4-nitrophenyl)pyrimidine

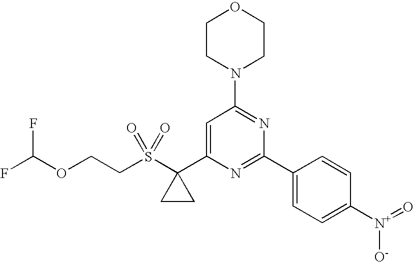

A solution of 2,2-difluoro-2-(fluorosulfonyl)acetic acid (0.856 mL, 8.29 mmol) in acetonitrile (4 mL) was added dropwise over 1 hour to a stirred solution of 2-[1-[6-morpholin-4-yl-2-(4-nitrophenyl)pyrimidin-4-yl]cyclopropyl]sulfonylethanol (480 mg, 1.10 mmol) and copper(I) iodide (742 mgl, 0.22 mmol) in acetonitrile (15 mL) at 55° C. under a nitrogen atmosphere. The mixture was stirred at 55° C. for 90 minutes, allowed to cool and diluted with ethyl acetate (300 mL) and washed with water (100 mL). The organic layer was dried over Na₂SO₄, filtered and evaporated to afford crude product. The crude product was purified by flash silica chromatography, elution gradient 10 to 50% ethyl acetate in isohexane, to give the desired material as a yellow solid (195 mg).

NMR Spectrum: ¹H NMR (400.132 MHz, DMSO-d₆) δ 1.59-1.68 (2H, m), 1.70-1.78 (2H, m), 3.70-3.81 (8H, m), 3.88-3.96 (2H, m), 4.28-4.35 (2H, m), 7.01 (1H, s), 8.33 (2H, d), 8.56 (2H, d)

LCMS Spectrum: m/z (ESI+) (M+H)+=485; HPLC tR=2.73 min.

### 2-[1-[6-Mrpholin-4-yl-2-(4-nitrophenyl)pyrimidin-4-yl]cyclopropyl]sulfonylethanol

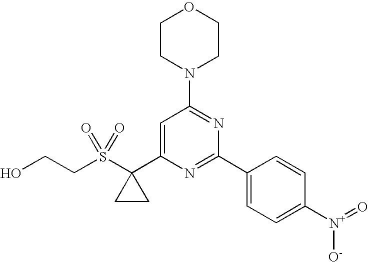

Bis(triphenylphosphine)palladium(II) chloride (84 mg, 0.12 mmol) was added to 2-[1-(2-chloro-6-morpholin-4-ylpyrimidin-4-yl)cyclopropyl]sulfonylethoxy-tri(propan-2-yl)silane (900 mg, 1.79 mmol), 4-(4,4,5,5-tetramethyl-1,3,2-dioxaborolan-2-yl)nitrobenzene (889 mg, 3.57 mmol) and 2M aqueous solution of sodium carbonate (3 mL, 6.00 mmol) in a solvent mixture of DME (25 mL) and water (1 mL). The mixture was stirred at 90° C. for 18 hours under an inert atmosphere. The reaction mixture was diluted with ethyl acetate (100 mL), and washed with water (2×100 mL). The organic layer was dried over Na₂SO₄, filtered and evaporated to afford crude product. The crude product was dissolved in DCM then 1M solution of tetrabutylammonium fluoride (8.93 mL, 8.93 mmol) added and left to stir 1 hour. A saturated aqueous solution of ammonium chloride was added, the layers separated and the organics dried over Na₂SO₄, filtered and evaporated. The crude product was purified by flash silica chromatography, elution gradient 10 to 70% ethyl acetate in isohexane, to give a material which was further purified by ion exchange chromatography using an SCX column, eluting with 7M ammonia in methanol, and finally triturated with a mixture of diethyl ether and isohexane to give the desired material as a beige solid (670 mg).

NMR Spectrum: ¹H NMR (400.132 MHz, DMSO-d₆) δ 1.53-1.62 (2H, m), 1.65-1.73 (2H, m), 3.56-3.67 (2H, m), 3.66-3.82 (8H, m), 3.80-3.97 (2H, m), 5.01 (1H, t), 7.01 (1H, s), 8.34 (2H, d), 8.57 (2H, d)

LCMS Spectrum: m/z (ESI+) (M+H)+=435; HPLC tR=2.18 min.

The preparation of 2-[1-(2-chloro-6-morpholin-4-ylpyrimidin-4-yl)cyclopropyl]sulfonylethoxy-tri(propan-2-yl)silane was described earlier.

### EXAMPLE 98

The following samples were prepared by heating a mixture of the carbamate (1 equivalent), triethylamine (4 equivalents) and the amine (4 equivalents) in NMP (2 mL) at 50° C. for 2 hours. The compounds were purified by preparative HPLC.

The following compounds were prepared in an analogous fashion from phenyl N-[4-[4-[(3S)-3-methylmorpholin-4-yl]-6-[1-[(4-methyl-1,3-thiazol-2-yl)sulfonyl]cyclobutyl]pyrimidin-2-yl]phenyl]carbamate or phenyl N-[4-[4-[(3S)-3-methylmorpholin-4-yl]-6-[1-(1,3-thiazol-2-ylsulfonyl)cyclobutyl]pyrimidin-2-yl]phenyl]carbamate and the appropriate amine.

| | | | | Retention |
|---|---|---|---|---|
| | | | LCMS | time |
| Example | Structure | NAME | MH+ | (min) |
| 98a | 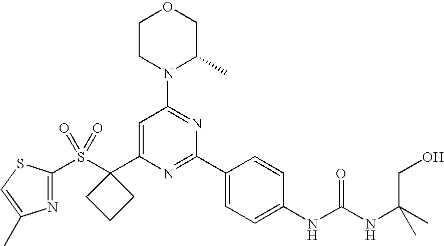 | 3-(1-hydroxy-2-methylpropan-2-yl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea | 601 | 2.49 |
| 98b | 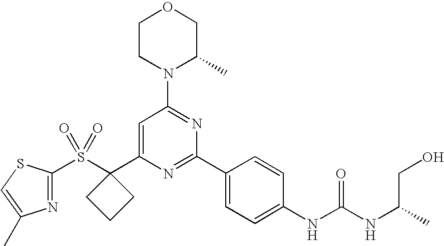 | 3-[(2S)-1-hydroxypropan-2-yl]-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea | 587 | 2.25 |
| 98c | 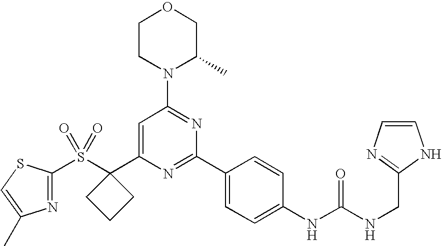 | 3-(1H-imidazol-2-ylmethyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea | 609 | 2.22 |
| 98d | 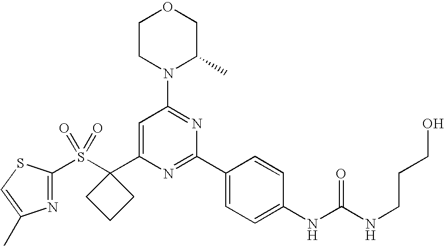 | 3-(3-hydroxypropyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea | 587 | 2.18 |
| 98e | 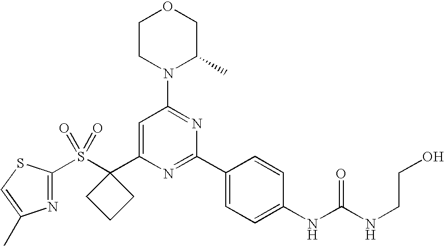 | 3-(2-hydroxyethyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea | 573 | 2.14 |
| 98f | 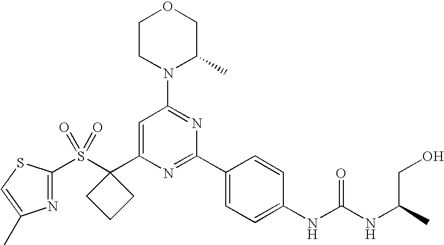 | 3-[(2R)-1-hydroxypropan-2-yl]-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea | 587 | 2.24 |
| 98g | 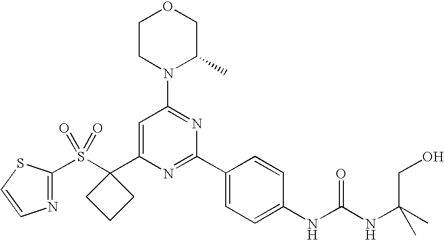 | 3-(1-hydroxy-2-methylpropan-2-yl)-1-[4-[4-[(3S)-3-methylmorpholin-4-yl]-6-[1-(1,3-thiazol-2-ylsulfonyl)cyclobutyl]pyrimidin-2-yl]phenyl]urea | 587 | 2.41 |
| 98h | 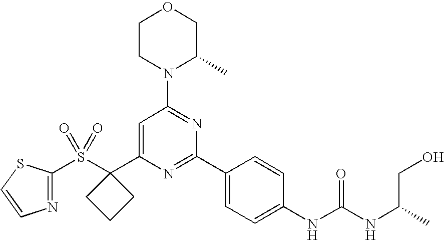 | 3-[(2S)-1-hydroxypropan-2-yl]-1-[4-[4-[(3S)-3-methylmorpholin-4-yl]-6-[1-(1,3-thiazol-2-ylsulfonyl)cyclobutyl]pyrimidin-2-yl]phenyl]urea | 573 | 2.20 |
| 98i | 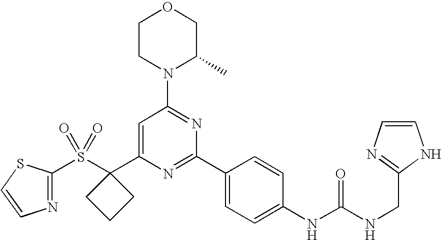 | 3-(1H-imidazol-2-ylmethyl)-1-[4-[4-[(3S)-3-methylmorpholin-4-yl]-6-[1-(1,3-thiazol-2-ylsulfonyl)cyclobutyl]pyrimidin-2-yl]phenyl]urea | 595 | 2.17 |
| 98j | 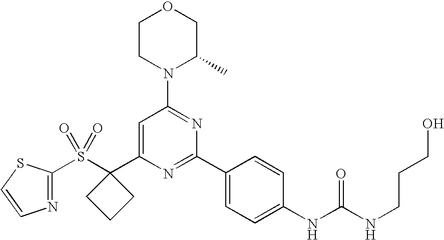 | 3-(3-hydroxypropyl)-1-[4-[4-[(3S)-3-methylmorpholin-4-yl]-6-[1-(1,3-thiazol-2-ylsulfonyl)cyclobutyl]pyrimidin-2-yl]phenyl]urea | 573 | 2.13 |
| 98k | 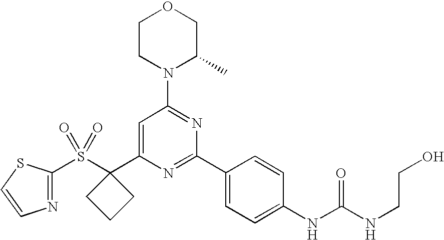 | 3-(2-hydroxyethyl)-1-[4-[4-[(3S)-3-methylmorpholin-4-yl]-6-[1-(1,3-thiazol-2-ylsulfonyl)cyclobutyl]pyrimidin-2-yl]phenyl]urea | 559 | 2.09 |
| 98l | 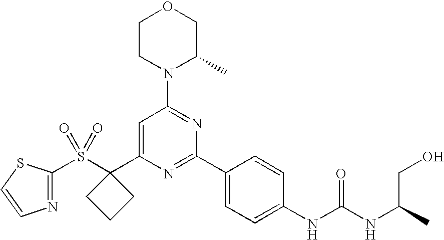 | 3-[(2R)-1-hydroxypropan-2-yl]-1-[4-[4-[(3S)-3-methylmorpholin-4-yl]-6-[1-(1,3-thiazol-2-ylsulfonyl)cyclobutyl]pyrimidin-2-yl]phenyl]urea | 573 | 2.18 |

### EXAMPLE 98a

¹H NMR (400.132 MHz, DMSO-d₆) δ 1.21 (3H, d), 1.24 (6H, s), 1.83-2.01 (1H, m), 2.12-2.20 (1H, m), 2.37 (3H, s), 2.83-2.92 (2H, m), 3.10-3.22 (4H, m), 3.36-3.41 (1H, m), 3.49 (1H, td), 3.64 (1H, d), 3.76 (1H, d), 3.97 (1H, d), 4.13 (1H, d), 4.48 (1H, s), 4.95 (1H, t), 5.99 (1H, s), 6.54 (1H, s), 7.36 (2H, d), 7.68 (1H, s), 7.86 (2H, d), 8.71 (1H, s)

### EXAMPLE 98b

¹H NMR (400.132 MHz, DMSO-d₆) δ 1.08 (3H, d), 1.21 (3H, d), 1.88-1.99 (1H, m), 2.12-2.21 (1H, m), 2.82-2.92 (2H, m), 3.10-3.23 (2H, m), 3.31 (3H, s), 3.32-3.42 (3H, m), 3.44-3.54 (1H, m), 3.61-3.78 (3H, m), 3.97 (1H, d), 4.09-4.17 (1H, m), 4.49 (1H, s), 4.78 (1H, t), 6.08 (1H, d), 6.54 (1H, s), 7.39 (2H, d), 7.68 (1H, s), 7.87 (2H, d), 8.68 (1H, s)

### EXAMPLE 98c

¹H NMR (400.132 MHz, DMSO-d₆) δ 1.21 (3H, d), 1.89-1.99 (1H, m), 2.11-2.20 (1H, m), 2.37 (3H, s), 2.85-2.95 (2H, m), 3.10-3.22 (3H, m), 3.43-3.54 (1H, m), 3.64 (1H, d), 3.76 (1H, d), 3.97 (1H, d), 4.14 (1H, d), 4.33 (2H, s), 4.50 (1H, s), 6.53-6.63 (2H, m), 6.93-7.05 (1H, m), 7.43 (2H, d), 7.68 (1H, s), 7.85-7.93 (2H, m), 8.90 (1H, s)

### EXAMPLE 98d

¹H NMR (400.132 MHz, DMSO-d₆) δ 1.21 (3H, d), 1.56-1.63 (2H, m), 1.86-1.97 (2H, m), 2.12-2.22 (2H, m), 2.77-2.92 (2H, m), 3.13-3.22 (3H, m), 3.25-3.34 (2H, m), 3.43-3.52 (3H, m), 3.64 (1H, d), 3.72-3.80 (1H, m), 3.97 (1H, d), 4.13 (1H, d), 4.44-4.53 (2H, m), 6.18 (1H, t), 6.54 (1H, s), 7.41 (2H, d), 7.68 (1H, s), 7.87 (2H, d), 8.68 (1H, s)

### EXAMPLE 98e

¹H NMR (400.132 MHz, DMSO-d₆) δ 1.21 (3H, d), 1.87-1.97 (1H, m), 2.15-2.21 (1H, m), 2.37 (3H, s), 2.79-2.94 (2H, m), 3.12-3.21 (3H, m), 3.27-3.34 (2H, m), 3.42-3.52 (3H, m), 3.64 (1H, d), 3.76 (1H, d), 3.97 (1H, d), 4.13 (1H, d), 4.49 (1H, s), 4.72 (1H, t), 6.24 (1H, t), 6.54 (1H, s), 7.40 (2H, d), 7.68 (1H, s), 7.87 (2H, d), 8.78 (1H, s)

### EXAMPLE 98f

¹H NMR (400.132 MHz, DMSO-d₆) δ 1.08 (3H, d), 1.21 (3H, d), 1.87-1.99 (1H, m), 2.12-2.23 (1H, m), 2.37 (3H, s), 2.83-2.92 (2H, m), 3.12-3.23 (4H, m), 3.34-3.41 (1H, m), 3.45-3.52 (1H, m), 3.64 (1H, d), 3.75 (1H, d), 3.97 (1H, d), 4.13 (1H, d), 4.50 (1H, s), 4.78 (1H, t), 6.05-6.10 (1H, m), 6.54 (1H, s), 7.40 (2H, d), 7.68 (1H, s), 7.87 (2H, d), 8.68 (1H, s)

### EXAMPLE 98g

¹H NMR (400.132 MHz, DMSO-d₆) δ 1.20 (3H, d), 1.25 (6H, s), 1.90-1.99 (1H, m), 2.14-2.21 (1H, m), 2.84-2.93 (2H, m), 3.17-3.25 (2H, m), 3.29-3.33 (1H, m), 3.45-3.52 (1H, m), 3.64 (1H, d), 3.76 (1H, d), 3.97 (1H, d), 4.13 (1H, d), 4.48 (1H, s), 4.95 (1H, t), 5.99 (1H, s), 6.53 (1H, s), 7.35 (2H, d), 7.83 (2H, d), 8.15 (1H, s), 8.70 (1H, s)

### EXAMPLE 98h

¹H NMR (400.132 MHz, DMSO-d₆) δ 1.08 (3H, d), 1.20 (3H, d), 1.91-2.00 (1H, m), 2.13-2.22 (1H, m), 2.85-2.95 (2H, m), 3.14-3.21 (2H, m), 3.30-3.42 (4H, m), 3.44-3.52 (1H, m), 3.64-3.78 (2H, m), 3.96 (1H, d), 4.13 (1H, d), 4.49 (1H, s), 4.78 (1H, t), 6.06-6.13 (1H, m), 6.53 (1H, s), 7.38 (2H, d), 7.85 (2H, d), 8.14 (1H, s), 8.67 (1H, s)

### EXAMPLE 98i

¹H NMR (400.132 MHz, DMSO-d₆) δ 1.21 (3H, d), 1.93-2.00 (1H, m), 2.13-2.20 (1H, m), 2.83-2.93 (2H, m), 3.14-3.24 (3H, m), 3.43-3.53 (1H, m), 3.64 (1H, d), 3.76 (1H, d), 3.96 (1H, d), 4.15 (1H, d), 4.32 (2H, s), 4.48 (1H, s), 6.54 (1H, s), 6.63 (1H, t), 6.93 (1H, s), 7.42 (2H, d), 7.87 (2H, d), 8.14 (1H, s), 8.90 (1H, s)

### EXAMPLE 98j

¹H NMR (400.132 MHz, DMSO-d₆) δ 1.20 (3H, d), 1.56-1.63 (2H, m), 1.88-2.01 (1H, m), 2.09-2.22 (1H, m), 2.84-2.94 (2H, m), 3.12-3.24 (5H, m), 3.43-3.54 (3H, m), 3.65 (1H, d), 3.76 (1H, d), 3.95 (1H, d), 4.06-4.20 (1H, m), 4.46-4.51 (1H, m), 6.19 (1H, t), 6.53 (1H, s), 7.39 (2H, d), 7.85 (2H, d), 8.15 (1H, s), 8.67 (1H, s)

### EXAMPLE 98k

¹H NMR (400.132 MHz, DMSO-d₆) δ 1.20 (3H, d), 1.90-2.00 (1H, m), 2.12-2.22 (1H, m), 2.84-2.94 (2H, m), 3.14-3.22 (3H, m), 3.25-3.34 (2H, m), 3.39-3.52 (3H, m), 3.63 (1H, d), 3.76 (1H, d), 3.96 (1H, d), 4.13 (1H, d), 4.48 (1H, s), 4.72 (1H, t), 6.24 (1H, t), 6.53 (1H, s), 7.39 (2H, d), 7.85 (2H, d), 8.15 (1H, s), 8.76 (1H, s)

### EXAMPLE 98l

¹H NMR (400.132 MHz, DMSO-d₆) δ 1.08 (3H, d), 1.20 (3H, d), 1.89-1.98 (1H, m), 2.12-2.23 (1H, m), 2.80-2.93 (2H, m), 3.14-3.24 (3H, m), 3.30-3.40 (2H, m), 3.45-3.53 (1H, m), 3.60-3.77 (3H, m), 3.96 (1H, d), 4.13 (1H, d), 4.49 (1H, s), 4.78 (1H, t), 6.08 (1H, d), 6.53 (1H, s), 7.38 (2H, d), 7.85 (2H, d), 8.15 (1H, s), 8.67 (1H, s)

The preparation of phenyl N-[4-[4-[(3S)-3-methylmorpholin-4-yl]-6-[1-[(4-methyl-1,3-thiazol-2-yl)sulfonyl]cyclobutyl]pyrimidin-2-yl]phenyl]carbamate is described below.

### Phenyl N-[4-[4-[(3S)-3-methylmorpholin-4-yl]-6-[1-[(4-methyl-1,3-thiazol-2-yl)sulfonyl]cyclobutyl]pyrimidin-2-yl]phenyl]carbamate

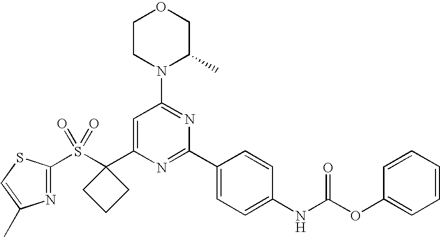

Sodium hydrogen carbonate (0.311 g, 3.71 mmol) was added to 4-[4-[(3S)-3-methylmorpholin-4-yl]-6-[1-[(4-methyl-1,3-thiazol-2-yl)sulfonyl]cyclobutyl]pyrimidin-2-yl]aniline (1.2 g, 2.47 mmol) in dioxane (30 mL) at 5° C. under nitrogen. Phenyl chloroformate (0.466 mL, 3.71 mmol) was then added and the resulting mixture stirred at RT for 2 hours. The reaction mixture was diluted with ethyl acetate (100 mL), washed with water (75 mL) and the organic layer dried over Na₂SO₄, filtered and evaporated to afford crude product. The crude gum was triturated with a mixture of diethyl ether and isohexane to give the desired material as a cream solid (1.2 g).

NMR Spectrum: ¹H NMR (400.132 MHz, DMSO-d₆) δ 1.21 (3H, d), 1.88-2.00 (1H, m), 2.10-2.21 (1H, m), 2.36 (3H, s), 2.83-2.95 (2H, m), 3.10-3.24 (3H, m), 3.44-3.53 (1H, m), 3.64 (1H, d), 3.76 (1H, d), 3.97 (1H, d), 4.15 (1H, d), 4.52 (1H, s), 6.59 (1H, s), 7.21-7.32 (3H, m), 7.39-7.47 (2H, m), 7.55 (2H, d), 7.68 (1H, s), 7.97 (2H, d), 10.39 (1H, s)

LCMS Spectrum: m/z (ESI+) (M+H)+=606; HPLC tR=3.12 min.

### 4-[4-[(3S)-3-Methylmorpholin-4-yl]-6-[1-[(4-methyl-1 3-thiazol-2-yl)sulfonyl]cyclobutyl]pyrimidin-2-yl]aniline

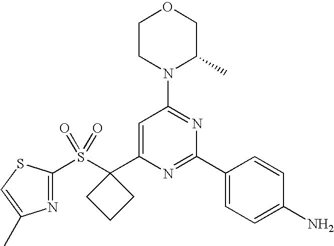

Bis(triphenylphosphine)palladium(II) chloride (0.115 g, 0.16 mmol) was added to 2-chloro-4-[(3S)-3-methylmorpholin-4-yl]-6-[1-[(4-methyl-1,3-thiazol-2-yl)sulfonyl]cyclobutyl]pyrimidine (1.4 g, 3.26 mmol), 4-(4,4,5,5-tetramethyl-1,3,2-dioxaborolan-2-yl)aniline (1.073 g, 4.90 mmol) and 2M aqueous odium carbonate solution (5 mL, 10.00 mmol) in a solvent mixture of DMF (6 mL), DME (12 mL), ethanol (3 mL) and water (3.5 mL) and the resulting mixture stirred at 95° C. for 5 hours under an inert atmosphere. The reaction mixture was diluted with ethyl acetate (250 mL), and washed with water (2×150 mL). The organic layer was dried over Na₂SO₄, filtered and evaporated to afford crude product. The crude product was purified by flash silica chromatography, elution gradient 20 to 60% ethyl acetate in isohexane, to give the desired material as a beige solid (1.17 g).

NMR Spectrum: ¹H NMR (400.132 MHz, DMSO-d₆) δ 1.19 (3H, d), 1.92 (1H, m), 1.99 (1H, s), 2.10-2.18 (1H, m), 2.39 (3H, s), 2.80-2.91 (2H, m), 3.09-3.24 (2H, m), 3.47 (1H, td), 3.62 (1H, d), 3.75 (1H, d), 3.95 (1H, d), 4.09 (1H, d), 4.45 (1H, s), 5.50 (2H, s), 6.43 (1H, s), 6.54 (2H, d), 7.61-7.80 (3H, m)

LCMS Spectrum: m/z (ESI+) (M+H)+=486; HPLC tR=2.51 min.

### 2-Chloro-4-[(3S)-3-methylmorpholin-4-yl]-6-[1-[(4-methyl-1 3-thiazol-2-yl)sulfonyl]cyclobutyl]pyrimidine

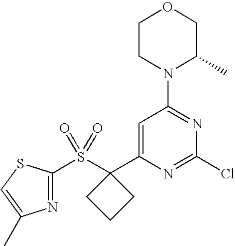

Tetrabutylammonium bromide (0.912 g, 2.83 mmol) was added to 2-chloro-4-[(3S)-3-methylmorpholin-4-yl]-6-[(4-methyl-1,3-thiazol-2-yl)sulfonylmethyl]pyrimidine (11 g, 28.29 mmol), 1,3-dibromopropane (17.23 mL, 169.71 mmol) and sodium hydroxide solution (50% w/w) (30 mL) in toluene (200 mL) and the resulting mixture stirred at 35° C. for 2.5 hours under a nitrogen atmosphere. The reaction mixture was diluted with ethyl acetate (200 mL), and washed with water (100 mL). The organic layer was dried over MgSO₄, filtered and evaporated to afford crude product. The crude product was purified by flash silica chromatography, elution gradient 10 to 50% ethyl acetate in isohexane, to give the desired material as a yellow gum (1.7 g).

NMR Spectrum: ¹H NMR (400.132 MHz, DMSO-d₆) δ 7.88 (1H, s), 1.18 (3H, d), 1.85-1.97 (1H, m), 2.04-2.18 (1H, m), 2.70-2.85 (2H, m), 3.00-3.21 (3H, m), 3.29 (3H, s), 3.37-3.49 (1H, m), 3.57 (1H, d), 3.71 (1H, d), 3.86-4.01 (2H, m), 4.34 (1H, s), 6.59 (1H, s) 7.91(1H,s).

LCMS Spectrum: m/z (ESI+) (M+H)+=429; HPLC tR=2.36 min.

The preparation of 2-chloro-4-[(3S)-3-methylmorpholin-4-yl]-6-[(4-methyl-1,3-thiazol-2-yl)sulfonylmethyl]pyrimidine was described earlier.

The preparation of phenyl N-[4-[4-[(3S)-3-methylmorpholin-4-yl]-6-[1-(1,3-thiazol-2-ylsulfonyl)cyclobutyl]pyrimidin-2-yl]phenyl]carbamate is described below.

### Phenyl N-[4-[4-[(3S)-3-methylmorpholin-4-yl]-6-[1-(1,3-thiazol-2-ylsulfonyl)cyclobutyl]pyrimidin-2-yl]phenyl]carbamate

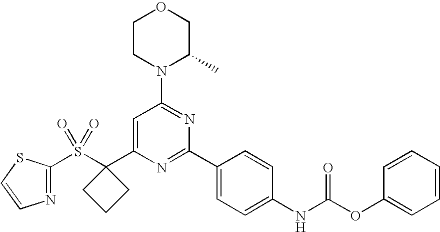

Sodium hydrogen carbonate (0.267 g, 3.18 mmol) was added to 4-[4-[(3S)-3-methylmorpholin-4-yl]-6-[1-(1,3-thiazol-2-ylsulfonyl)cyclobutyl]pyrimidin-2-yl]aniline (1 g, 2.12 mmol) in dioxane (30 mL) at 5° C. under nitrogen. Phenyl chloroformate (0.4 mL, 3.18 mmol) was then added. The resulting mixture was stirred at RT for 2 hours. The reaction mixture was diluted with ethyl acetate (100 mL) and washed with water (75 mL). The organic layer was dried over Na₂SO₄, filtered and evaporated to afford crude product. The crude gum was triturated with a mixture of diethyl ether and isohexane to give the desired material as a cream solid (0.80 g).

NMR Spectrum: ¹H NMR (400.132 MHz, DMSO-d₆) δ 1.21 (3H, d), 1.89-2.01 (1H, m), 2.12-2.22 (1H, m), 2.84-2.97 (2H, m), 3.11-3.26 (3H, m), 3.44-3.54 (1H, m), 3.64 (1H, d), 3.76 (1H, d), 3.97 (1H, d), 4.15 (1H, d), 4.50 (1H, s), 6.57 (1H, s), 7.15-7.35 (3H, m), 7.38-7.48 (2H, m), 7.49-7.60 (2H, m), 8.15 (2H, s), 10.39 (1H, s)

LCMS Spectrum: m/z (ESI+) (M+H)+=592; HPLC tR=3.06 min.

### 4-[4-[(3S)-3-Methylmorpholin-4-yl]-6-[1-(1,3-thiazol-2-ylsulfonyl)cyclobutylpyrimidin-2--yl]aniline

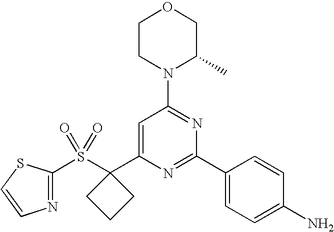

Bis(triphenylphosphine)palladium(II) chloride (0.093 g, 0.13 mmol) was added to 2-chloro-4-[(3S)-3-methylmorpholin-4-yl]-6-[1-(1,3-thiazol-2-ylsulfonyl)cyclobutyl]pyrimidine (1.1 g, 2.65 mmol), 4-(4,4,5,5-tetramethyl-1,3,2-dioxaborolan-2-yl)aniline (0.871 g, 3.98 mmol) and 2M aqueous sodium carbonate solution (5 mL, 10.00 mmol) in a solvent mixture of DMF (6 mL), DME (12 mL), ethanol (3 mL) and water (3.5 mL) and the resulting mixture stirred at 95° C. for 5 hours under an inert atmosphere. The reaction mixture was diluted with ethyl acetate (250 mL), and washed with water (2×150 mL). The organic layer was dried over Na₂SO₄, filtered and evaporated to afford crude product. The crude product was purified by flash silica chromatography, elution gradient 20 to 70% ethyl acetate in isohexane, to give the desired material as a yellow solid (0.99 g).

NMR Spectrum: ¹H NMR (400.132 MHz, DMSO-d₆) δ 1.18 (3H, d), 1.88-1.96 (1H, m), 2.11-2.21 (1H, m), 2.82-2.91 (2H, m), 3.05-3.23 (3H, m), 3.47 (1H, td), 3.62 (1H, d), 3.74 (1H, d), 3.95 (1H, d), 4.10 (1H, s), 4.44 (1H, s), 5.50 (2H, s), 6.42 (1H, s), 6.51 (2H, d), 7.70 (2H, d), 8.15 (2H, s)

### 2-Chloro-4-[(3S)-3-methylmorpholin-4-yl]-6-[1-(13-thiazol-2-ylsulfonyl)cyclobutyl]pyrimidine

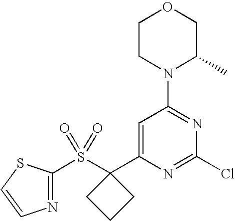

Tetrabutylammonium bromide (0.559 g, 1.73 mmol) was added to 2-chloro-4-[(3S)-3-methylmorpholin-4-yl]-6-(1,3-thiazol-2-ylsulfonylmethyl)pyrimidine (6.5 g, 17.34 mmol), 1,3-dibromopropane (10.56 mL, 104.04 mmol) and sodium hydroxide (50% w/w) (10 mL) in toluene (20 mL) and the resulting mixture stirred at 70° C. for 30 minutes. The reaction mixture was diluted with ethyl acetate (200 mL), and washed with water (100 mL). The organic layer was dried over MgSO₄, filtered and evaporated to afford crude product. The crude product was purified by flash silica chromatography, elution gradient 20 to 60% ethyl acetate in isohexane, to give the desired material as a yellow gum (1.0 g).

NMR Spectrum: ¹H NMR (400.132 MHz, DMSO-d₆) δ 1.86-1.97 (1H, m), 2.07-2.18 (1H, m), 2.72-2.84 (2H, m), 3.04-3.19 (3H, m), 3.42 (1H, td), 3.57 (1H, d), 3.70 (1H, d), 3.89-3.99 (2H, m), 4.33 (1H, s), 6.60 (1H, s), 8.19 (1H, d), 8.30 (1H, d)

The preparation of 2-chloro-4-[(3S)-3-methylmorpholin-4-yl]-6-(1,3-thiazol-2-ylsulfonylmethyl)pyrimidine was described earlier.

### EXAMPLE 99

The following samples were prepared by heating a mixture of the aniline (1 equivalent) and 1,1 thiocarbonyldiimidazole (1.2 equivalents) in a mixture of DCM: THF (2: 1) at RT for 30 minutes then adding the amine (5 equivalents) and stirring at 50° C. for 2 hours. The compounds were purified by preparative HPLC.

The following compounds were prepared in an analogous fashion from either 3-[1-[2-(4-aminophenyl)-6-[(3S)-3-methylmorpholin-4-yl]pyrimidin-4-yl]cyclobutyl]sulfonylpropan-1-ol or 3-[1-[2-(4-aminophenyl)-6-[(3S)-3-methylmorpholin-4-yl]pyrimidin-4-yl]cyclopropyl]sulfonylpropan-1-ol and the appropriate amine.

| | | | | Retention |
|---|---|---|---|---|
| | | | LCMS | time |
| Example | Structure | NAME | MH+ | (min) |
| 99a | 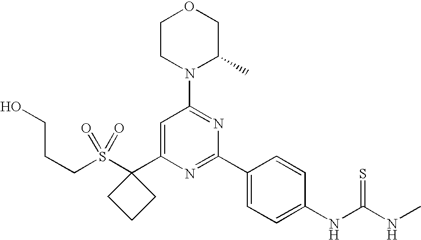 | 1-[4-[4-[1-(3-hydroxypropylsulfonyl)cyclobutyl]-6-[(3S)-3-methylmorpholin-4-yl]pyrimidin-2-yl]phenyl]-3-methylthiourea | 520 | 1.92 |
| 99b | 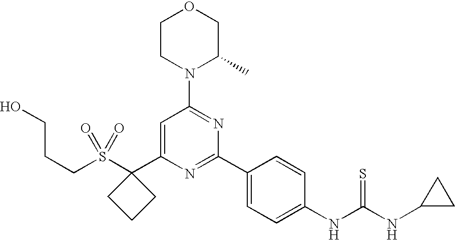 | 3-cyclopropyl-1-[4-[4-[1-(3-hydroxypropylsulfonyl)cyclobutyl]-6-[(3S)-3-methylmorpholin-4-yl]pyrimidin-2-yl]phenyl]thiourea | 546 | 2.07 |
| 99c | 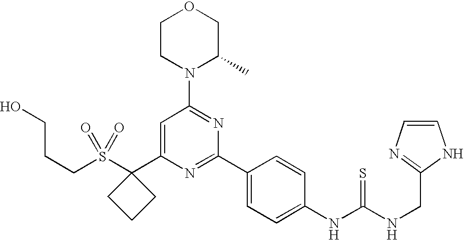 | 1-[4-[4-[1-(3-hydroxypropylsulfonyl)cyclobutyl]-6-[(3S)-3-methylmorpholin-4-yl]pyrimidin-2-yl]phenyl]-3-(1H-imidazol-2-ylmethyl)thiourea | 586 | 1.83 |
| 99d | 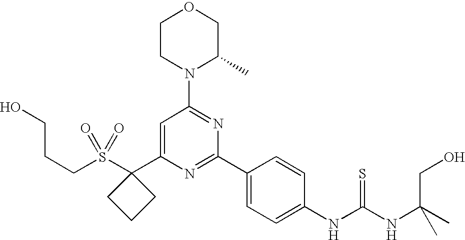 | 3-(1-hydroxy-2-methylpropan-2-yl)-1-[4-[4-[1-(3-hydroxypropylsulfonyl)cyclobutyl]-6-[(3S)-3-methylmorpholin-4-yl]pyrimidin-2-yl]phenyl]thiourea | 578 | 2.02 |
| 99e | 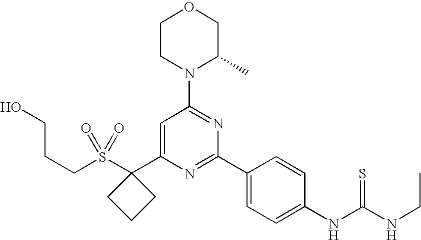 | 1-ethyl-3-[4-[4-[1-(3-hydroxypropylsulfonyl)cyclobutyl]-6-[(3S)-3-methylmorpholin-4-yl]pyrimidin-2-yl]phenyl]thiourea | 534 | 2.07 |
| 99f | 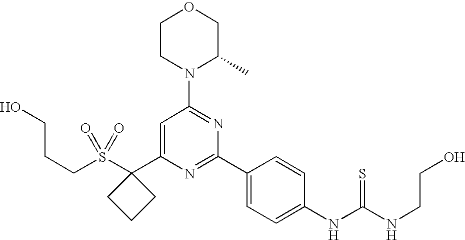 | 3-(2-hydroxyethyl)-1-[4-[4-[1-(3-hydroxypropylsulfonyl)cyclobutyl]-6-[(3S)-3-methylmorpholin-4-yl]pyrimidin-2-yl]phenyl]thiourea | 550 | 1.75 |
| 99g | 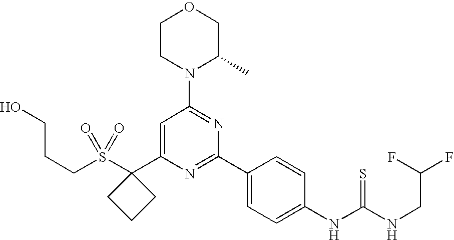 | 3-(2,2-difluoroethyl)-1-[4-[4-[1-(3-hydroxypropylsulfonyl)cyclobutyl]-6-[(3S)-3-methylmorpholin-4-yl]pyrimidin-2-yl]phenyl]thiourea | 570 | 2.21 |
| 99h | 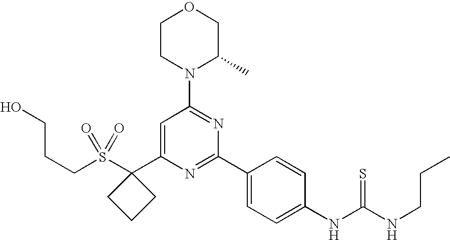 | 3-[4-[4-[1-(3-hydroxypropylsulfonyl)cyclobutyl]-6-[(3S)-3-methylmorpholin-4-yl]pyrimidin-2-yl]phenyl]-1-propylthiourea | 548 | 2.38 |
| 99i | 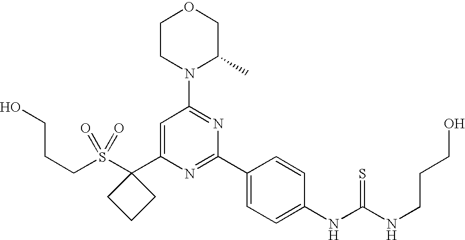 | 3-(3-hydroxypropyl)-1-[4-[4-[1-(3-hydroxypropylsulfonyl)cyclobutyl]-6-[(3S)-3-methylmorpholin-4-yl]pyrimidin-2-yl]phenyl]thiourea | 564 | 1.91 |
| 99j | 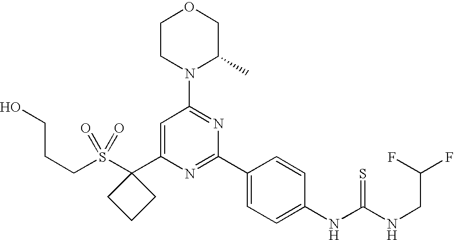 | 3-(2,2-difluoroethyl)-1-[4-[4-[1-(3-hydroxypropylsulfonyl)cyclopropyl]-6-[(3S)-3-methylmorpholin-4-yl]pyrimidin-2-yl]phenyl]thiourea | 556 | 2.06 |

### EXAMPLE 99a

¹H NMR (400.132 MHz, DMSO-d₆) δ 1.24 (3H, d), 1.73-1.81 (2H, m), 1.88-1.97 (1H, m), 2.03-2.10 (1H, m), 2.76-2.87 (2H, m), 2.89-3.05 (7H, m), 3.19-3.26 (1H, m), 3.36-3.41 (2H, m), 3.46-3.55 (1H, m), 3.66 (1H, d), 3.77 (1H, d), 3.98 (1H, d), 4.23 (1H, d), 4.52-4.61 (2H, m), 6.76 (1H, s), 7.55 (2H, d), 7.85 (1H, s), 8.28 (2H, d), 9.73 (1H, s)

### EXAMPLE 99b

¹H NMR (400.132 MHz, DMSO-d₆) δ 0.56-0.64 (2H, m), 0.73-0.80 (2H, m), 1.25 (3H, d), 1.72-1.81 (2H, m), 1.88-1.96 (1H, m), 2.03-2.10 (1H, m), 2.80-3.04 (6H, m), 3.17-3.26 (1H, m), 3.34-3.41 (2H, m), 3.45-3.57 (1H, m), 3.66 (1H, d), 3.77 (1H, d), 3.98 (1H, d), 4.23 (1H, d), 4.49-4.64 (2H, m), 6.76 (1H, s), 7.59-7.66 (2H, m), 7.96 (1H, s), 8.28 (2H, d), 9.51 (1H, s)

### EXAMPLE 99c

¹H NMR (400.132 MHz, DMSO-d₆) δ 1.25 (3H, d), 1.73-1.82 (2H, m), 1.89-1.97 (1H, m), 2.04-2.11 (1H, m), 2.78-2.88 (2H, m), 2.92-2.99 (2H, m), 2.98-3.05 (2H, m), 3.20-3.26 (1H, m), 3.34-3.42 (2H, m), 3.46-3.57 (1H, m), 3.66 (1H, d), 3.77 (1H, d), 3.98 (1H, d), 4.04-4.10 (1H, m), 4.25 (1H, d), 4.54-4.63 (2H, m), 4.71 (2H, s), 6.76 (1H, s), 6.99 (2H, s), 7.69 (2H, d), 8.21 (1H, s), 8.29 (2H, d), 10.03 (1H, s)

### EXAMPLE 99d

¹H NMR (400.132 MHz, DMSO-d₆) δ 1.25 (3H, d), 1.45 (6H, s), 1.73-1.80 (2H, m), 1.87-1.95 (1H, m), 2.03-2.10 (1H, m), 2.79-2.87 (2H, m), 2.92-2.98 (2H, m), 2.97-3.05 (2H, m), 3.20-3.24 (1H, m), 3.35-3.42 (2H, m), 3.48-3.57 (3H, m), 3.66 (1H, d), 3.77 (1H, d), 3.95-4.01 (1H, m), 4.03-4.09 (1H, m), 4.25 (1H, d), 4.52-4.62 (2H, m), 6.75 (1H, s), 7.61 (2H, d), 8.27 (2H, d), 9.92 (1H, s)

### EXAMPLE 99e

¹H NMR (400.132 MHz, DMSO-d₆) δ 1.14 (3H, t), 1.24 (3H, d), 1.72-1.82 (2H, m), 1.88-1.98 (1H, m), 2.01-2.11 (1H, m), 2.76-2.87 (2H, m), 2.89-2.98 (2H, m), 2.98-3.06 (2H, m), 3.21-3.27 (1H, m), 3.35-3.42 (2H, m), 3.46-3.56 (3H, m), 3.66 (1H, d), 3.77 (1H, d), 3.98 (1H, d), 4.24 (1H, d), 4.52-4.62 (2H, m), 6.75 (1H, s), 7.57 (2H, d), 7.89 (1H, s), 8.28 (2H, d), 9.63 (1H, s)

### EXAMPLE 99f

¹H NMR (400.132 MHz, DMSO-d₆) δ 1.24 (3H, d), 1.73-1.81 (2H, m), 1.86-1.97 (1H, m), 2.02-2.12 (1H, m), 2.79-2.87 (2H, m), 2.92-2.99 (2H, m), 2.97-3.05 (2H, 5 m), 3.20-3.28 (1H, m), 3.35-3.43 (2H, m), 3.47-3.60 (3H, m), 3.66 (1H, d), 3.77 (1H, d), 3.98 (1H, d), 4.25 (1H, d), 4.50-4.63 (2H, m), 4.81 (1H, s), 6.75 (1H, s), 7.63 (2H, d), 7.88 (1H, s), 8.28 (2H, d), 9.81 (1H, s)

### EXAMPLE 99g

¹H NMR (400.132 MHz, DMSO-d₆) δ 10.01 (1H, s), 8.31 (2H, d), 8.12 (1H, s), 7.59 (2H, d), 6.77 (1H, s), 6.39-6.03 (1H, m), 4.64-4.54 (2H, m), 4.25 (1H, d), 4.05-3.94 (3H, m), 3.77 (1H, d), 3.66 (1H, d), 3.56-3.46 (1H, m), 3.42-3.36 (2H, m), 3.27-3.19 (1H, m), 3.05-2.98 (2H, m), 2.98-2.91 (2H, m), 2.89-2.79 (2H, m), 2.12-1.98 (1H, m), 1.97-1.87 (1H, m), 1.81-1.73 (2H, m), 1.25 (3H, d)

### EXAMPLE 99h

¹H NMR (400.132 MHz, DMSO-d₆) δ 10.01 (1H, s), 8.31 (2H, d), 8.12 (1H, s), 7.59 (2H, d), 6.77 (1H, s), 6.39-6.03 (1H, m), 4.64-4.54 (2H, m), 4.25 (1H, d), 4.05-3.94 (3H, m), 3.77 (1H, d), 3.66 (1H, d), 3.56-3.46 (1H, m), 3.42-3.36 (2H, m), 3.27-3.19 (1H, m), 3.05-2.98 (2H, m), 2.98-2.91 (2H, m), 2.89-2.79 (2H, m), 2.12-1.98 (1H, m), 1.97-1.87 (1H, m), 1.81-1.73 (2H, m), 1.25 (3H, d)

### EXAMPLE 99i

¹H NMR (400.132 MHz, DMSO-d₆) δ 1.24 (3H, d), 1.69-1.80 (4H, m), 1.88-1.98 (1H, m), 2.03-2.09 (1H, m), 2.78-2.89 (2H, m), 2.90-2.97 (2H, m), 2.98-3.05 (2H, m), 3.17-3.25 (1H, m), 3.35-3.42 (2H, m), 3.46-3.59 (5H, m), 3.66 (1H, d), 3.77 (1H, d), 3.98 (1H, d), 4.25 (1H, d), 4.52-4.61 (2H, m), 6.75 (1H, s), 7.58 (2H, d), 7.90 (1H, s), 8.28 (2H, d), 9.69 (1H, s)

### EXAMPLE 99j

Spectrum not recorded.

The preparations of 3-[1-[2-(4-aminophenyl)-6-[(3S)-3-methylmorpholin-4-yl]pyrimidin-4-yl]cyclobutyl]sulfonylpropan-1-ol and 3-[1-[2-(4-aminophenyl)-6-[(3S)-3-methylmorpholin-4-yl]pyrimidin-4-yl]cyclopropyl]sulfonylpropan-1-ol were described earlier.

1. A compound of formula (I) 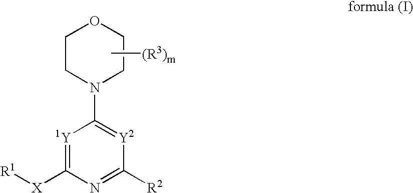
or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═C⁵CR⁶R⁷—, —CR⁶R⁷CR⁵═CR⁴—, —C═CCR⁶R⁷—, —CR⁶R⁷C═C—, —NR⁴CR⁶R⁷, —OCR⁶R⁷, —SCR⁶R⁷—, —S(O)CR⁶R⁷—, —S(O)₂CR⁶R⁷, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷— and —S(O)₂NR⁴CR⁶R⁷;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is independently selected from halo, cyano, nitro, —R¹³, —OR¹³, —SR¹³, —SOR³, —SO₂R¹³, —CO₂R¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 4- to 10-membered carbocyclic or heterocyclic ring wherein 1, 2 or 3 ring carbon atoms is optionally replaced with N, 0 or S and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹², R¹⁷ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylaamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹⁹ is hydrogen, cyano or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; or R¹⁸ and R¹⁹ together with the nitrogen atom to which they are attached form a 3- to 10-membered heterocyclic ring wherein 1 or 2 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

2. The Acompound according to claim 1, or a pharmaceutically acceptable salt thereof, wherein
m is 0, 1 or 2;
¹Y and Y² are independently N or CR⁸ provided that one of ¹Y and Y² is N and the other is CR⁸
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R—, —NR⁴C(O)CR⁶R—, —NR⁴C(O)NR⁵CR⁶R⁷— and —S(O)₂NR⁴CR⁶R⁷;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
or X—R¹ is —CR⁶R⁷OH;
R² is a group selected from carbocyclyl and heterocyclyl which group is optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², —NR¹¹COCONR¹²R¹⁶, —NR¹¹SO₂R¹², —NR¹⁷CONR¹⁸R¹⁹ and —NR¹⁷CSNR¹⁸R¹⁹;
each R³, when present, is selected from cyano, R¹³, and —CONR¹³R¹⁴;
R⁴ and R⁵ are independently hydrogen or C₁₋₆alkyl;
or, when X is —NR⁴CR⁵R⁶—, —NR⁴C(O)CR⁶R⁷— or —NR⁴C(O)NR⁵CR⁶R⁷—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, oxo, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkyl amino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ together with the carbon atom to which they are attached form a 3- to 10-membered carbocyclic ring or heterocyclic ring wherein 1 ring carbon atom is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹², R¹⁷ and R¹⁸ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹³and R¹⁴ are independently hydrogen or a C₁₋₃alkyl which is optionally substituted by one or more substituent groups selected from halo, cyano, hydroxy and C₁₋₃alkoxy; and
R¹⁹ is hydrogen, cyano or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
or R¹⁸ and R¹⁹ together with the nitrogen atom to which they are attached form a 6-membered heterocyclic ring wherein 1 ring carbon atoms is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

3. The compound according to claim 1, or a pharmaceutically acceptable salt thereof, wherein ¹Y is CH and Y² is N.

4. The compound according to claim 1, or a pharmaceutically acceptable salt thereof, wherein X is a —S(O)₂CR⁶R⁷— linker group.

5. The compound according to claim 1, or a pharmaceutically acceptable salt thereof, wherein R¹ is a group selected from methyl, ethyl, propyl, butyl, isobutyl, tert-butyl, cyclopropyl, cyclopentyl cyclohexyl, phenyl, benzyl, phen ethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH₂ and —CONHCH₃.

6. The compound according to claim 1, or a pharmaceutically acceptable salt thereof, wherein R² is 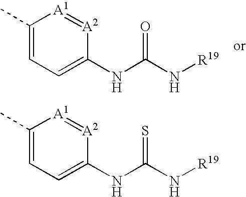
wherein A¹ and A² are selected from CH or N provided that at least one of A¹ or A² is CH.

7. The compound according to claim 6, or a pharmaceutically acceptable salt thereof, wherein R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyrazolylmethyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(CI-6alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

8. The compound according to claim 1, or a pharmaceutically acceptable salt thereof, wherein R⁶ and R⁷ together with the carbon atom to which they are attached form a 3- to 5-membered carbocyclic ring.

9. The compound according to claim 1, or a pharmaceutically acceptable salt thereof, wherein the compound of formula (I) is a compound of formula (IA), (IB) or (IC) 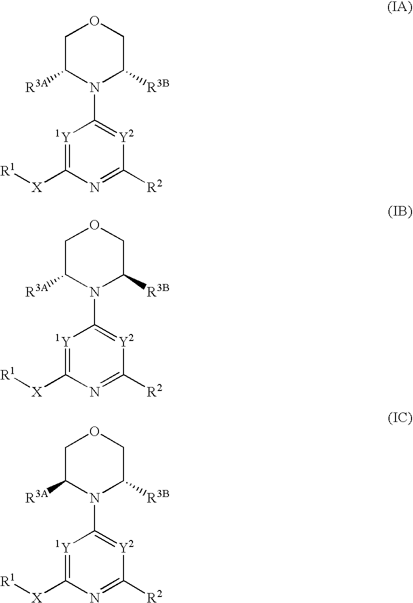
wherein
X is a —S(O)₂CR⁶R⁷— linker group;
Y is CH and Y² is N;
R¹ is a group selected from methyl, ethyl, isopropyl, tert-butyl, cyclopropyl, cyclopentyl, cyclohexyl, —CH₂CH₂OH, —CH₂CH₂CH₂H, —CH₂CH₂C(OH)(CH₃)₂, —CH₂CH₂CH₂OCHF₂, —CH₂CH₂OCH₃, —CH₂CH₂NHC(O)CH₃, —CH₂C(O)NH₂, —CH₂C(O)NHMe, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-(trifluoromethyl)phenyl, 2-methylphenyl, 4-methylphenyl, 4-(2-hydroxyethylamino)phenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1-(difluoromethyl)-3,5-dimethylpyrazol-4-yl, 1,3-dimethylpyrazol-4-yl, pyridin-4-yl, pyridin-2-yl, 5-fluoropyridin-2-yl, 5-fluoropyridin-3-yl, thiazol-2-yl, 4-methylthiazol-2-yl, 4,5-dimethylthiazol-2-yl, 2,4-dimethylthiazol-5-yl, 5-methyl-1,3,4-thiadiazol-2-yl, terahydrofuran-3-yl and terahydropyran-4-yl;
R² is 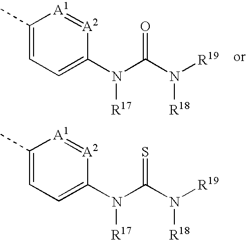
wherein A¹ and A² are CH;
R¹⁷ is hydrogen;
R¹⁸ is hydrogen; and
R¹⁹ is is hydrogen, cyano or a group selected from methyl, ethyl, propyl, i-propyl, i-butyl, t-butyl, cyclopropyl, cyclobutyl, —CH₂(cyclopropyl), —CH₂CH₂NMe₂, —CH(CH₃)CH₃OH, —C(CH₃)₂CH₃OH, —CH₂C(CH₃)₃OH, —CH₂C(CH₃)₂CH₃OH, —CH₂CH₃OH, —CH₂CH₂CH₂OH, —CH₂CF₃, —CH₂CHF₂, —CH₂CH₂F, —CH₂CH₂Cl, —CH₂CH₂SO₂Me, —CH₂CH(OH)CF₃, —CH₂CH₂CN, —CH₂CN, —CH₂CONMe₂, —CH₂CO₂H, 1-(methyl)cyclopropyl, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-(trifluoromethyl)phenyl, 4-fluorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 1,1-dioxothiolan-3-yl, 5-methylisoxazol-3-yl, —CH₂(1-methylpyrazol-4-yl), 1-methylpyrazol-4-yl, —CH₂(1-methylpyrazol-4-yl), 5-methylpyrazin-2-yl, —CH₂(2H-1,2,4-triazol-3-yl), 6-methoxypryridin-3-yl, pyridin-2-yl, 5-fluoropyridin-2-yl, thiazol-2-yl, 1,2,4-thiadiazol-5-yl and 1-methylpyrazol-3-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl, ethyl, hydroxymethyl, dimethylcarbamoyl or carbamoyl; or
when R^{3′} is methyl, R³ is methyl.

10. The compound according to claim 1, or a pharmaceutically acceptable salt thereof, wherein the compound of formula (I) is a compound of formula (IA), (IB) or (IC) 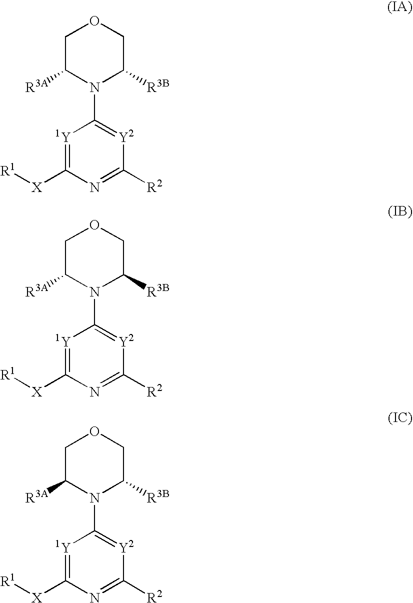
wherein
X is a —S(O)₂CR⁶R⁷— linker group;
¹Y is CH and Y2 is N;
R¹ is a group selected from methyl, ethyl, isopropyl, tert-butyl, cyclopropyl, —CH₂CH₃OH, —CH₂CH₂CH₂OH, —CH₂CH₂C(OH)(CH₃)₂, —CH₂CH₂CH₂OCHF₂, —CH₂CH₂OCH₃, —CH₂CH₂NHC(O)CH₃, —CH₂CH₂NHMe, phenyl, 2-fluorophenyl, 3-fluorophenyl, 4-fluorophenyl, 2-fluoro-4-methylaminophenyl, 4-fluoro-2-methylphenyl, 5-fluoro-2-methylphenyl, 3-fluoro-4-(2-hydroxyethylamino)phenyl, 4-(difluoromethoxy)phenyl, 4-carbamoyl-2-chlorophenyl, 4-chlorophenyl, 2-chlorophenyl, 3-chloro-4-fluorophenyl, 3-chloro-4-methylaminophenyl, 3-chloro-4-ethylaminophenyl, 3-chloro-4-(2-fluoroethylamino)phenyl, 3-chloro-4-(2-hydroxyethylamino)phenyl, 2-chloro-4-cyanophenyl, 4-cyanophenyl, 2,4-difluorophenyl, 2,5-difluorophenyl, 2,6-difluorophenyl, 3,4-difluorophenyl, 3,5-difluorophenyl, 2-methylphenyl, 4-methylphenyl, 1H-imidazol-2-yl, 3,5-dimethylisoxazol-4-yl, 2-(dimethylcarbamoyl)pyridin-3-yl, 5-(dimethylcarbamoyl)pyridin-2-yl, 1-(difluoromethyl)pyrazol-4-yl, 1,3-dimethylpyrazol-4-yl, pyridin-4-yl, pyridin-2-yl, 5-fluoropyridin-2-yl, thiazol-2-yl, 4-methylthiazol-2-yl, 4,5-dimethylthiazol-2-yl, 2,4-dimethylthiazol-5-yl and 5-methyl-1,3,4-thiadiazol-2-yl;
R² is 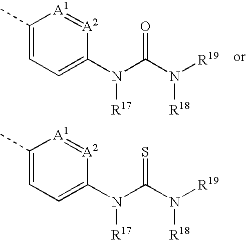
wherein A¹ and A² are CH;
R¹⁷ is hydrogen;
R¹⁸ is hydrogen; and
R¹⁹ is hydrogen or a group selected from methyl, ethyl, propyl, i-propyl, cyclopropyl, cyclobutyl, —CH₂(cyclopropyl), —CH₂CH₂NMe₂, —CH(CH₃)CH₃OH, —C(CH₃)₂CH₃OH, —CH₂CH₃OH, —CH₂CH₂CH₃OH, —CH₂CF₃, —CH₂CHF₂, —CH₂CH₂F, —CH₂CH₂Cl, —CH₂CH₂CN, —CH₂(1-hydroxycyclopropyl), 1-(hydroxymethyl)cyclopropyl, (1R)-2-hydroxy-1-methylethyl, (1S)-2-hydroxy-1-methylethyl, phenyl, 4-methylphenyl, 4-chlorophenyl, 4-methoxyphenyl, 3,4-difluorophenyl, —CH₂CH₂(pyrrolidin-1-yl), —CH₂(imidazol-2-yl), 1-methylimidazol-4-yl, oxazolyl-2-yl, isoxazolyl-3-yl, oxetan-3-yl, 5-methylisoxazol-3-yl, 1-methylpyrazol-4-yl, 5-methylpyrazin-2-yl, 6-methoxypryridin-3-yl, thiazol-2-yl, 1,2,4-thiadiazol-5-yl and 1-methylpyrazol-3-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl or ethyl; or
when R^{3A} is methyl, R^{3B} is methyl.

11. The compound according to claim 1, or a pharmaceutically acceptable salt thereof, wherein the compound of formula (I) is a compound of formula (IA), (IB) or (IC) 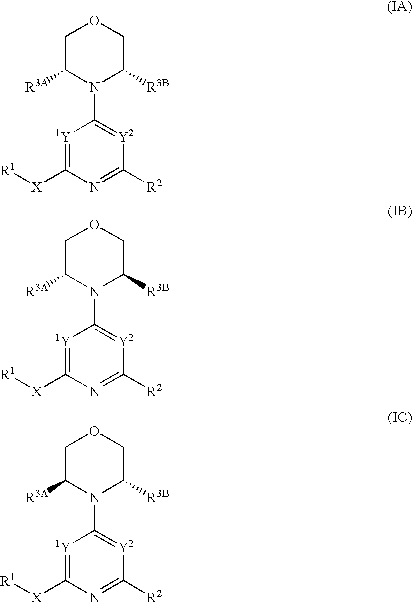
wherein
X is a —S(O)₂CR⁶R⁷— linker group;
¹Y is CH and Y² is N;
R¹ is a group selected from methyl, ethyl, cyclopropyl, —CH₂CH₂CH₃OH, phenyl, 2-fluorophenyl, 3-fluorophenyl, 4-fluorophenyl, 2-chlorophenyl, 2-methylphenyl, 5-fluoropyridin-2-yl, pyridin-2-yl, thiazol-2-yl and 4-methylthiazol-2-yl;
R² is 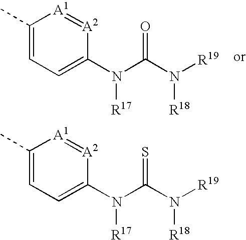
wherein A¹ and A² are CH;
R¹⁷ is hydrogen;
R¹⁸ is hydrogen; and
R¹⁹ is a group selected from methyl, ethyl, cyclopropyl, cyclobutyl, —CH(CH₃)CH₃OH, —CH₂CH₃OH, —CH₂CH₂CH₃OH, —CH₂CHF₂, —CH₂CH₂F, —CH₂CH₂CN, (1R)-2-hydroxy-1-methylethyl, (1S)-2-hydroxy-1-methylethyl, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is hydrogen, methyl or ethyl; or
when R^{3A} is methyl, R^{3B} is methyl.

12. The compound according to claim 1, or a pharmaceutically acceptable salt thereof, wherein the compound of formula (I) is a compound of formula (Ia), (lb) or (Ic) 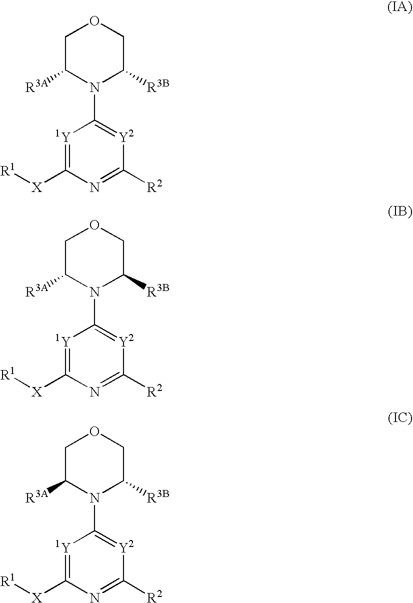
or a pharmaceutically acceptable salt thereof;
wherein
X is a —S(O)₂CR⁶R⁷— linker group;
¹Y is CH and Y² is N;
R¹ is a group selected from methyl, ethyl, cyclopropyl, —CH₂CH₂CH₂OH, phenyl, 2-fluorophenyl, 3-fluorophenyl, 4-fluorophenyl, 2-chlorophenyl, 2-methylphenyl, 5-fluoropyridin-2-yl, pyridin-2-yl, thiazol-2-yl and 4-methylthiazol-2-yl;
R² is 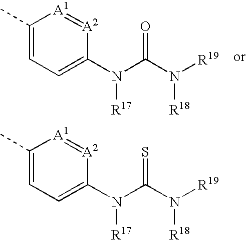
wherein A¹ and A² are CH;
R¹⁷ is hydrogen;
R¹⁸ is hydrogen; and
R¹⁹ is a group selected from methyl, ethyl, cyclopropyl, cyclobutyl, —CH(CH₃)CH₃OH, —CH₂CH₂OH, —CH₂CH₂CH₃OH, —CH₂CHF₂, —CH₂CH₂F, —CH₂CH₂CN, (1R)-2-hydroxy-1-methylethyl, (1S)-2-hydroxy-1-methylethyl, —CH₂(imidazol-2-yl), oxazolyl-2-yl, isoxazolyl-3-yl, 1-methylpyrazol-4-yl, 5-methylpyrazin-2-yl, thiazol-2-yl and 1,2,4-thiadiazol-5-yl;
R⁶ and R⁷ together with the carbon atom to which they are attached form a cyclopropyl, cyclobutyl, cyclopentyl, tetrahydropyranyl or piperidyl ring; and
when R^{3A} is hydrogen, R^{3B} is methyl or ethyl; or
when R^{3A} is methyl, R^{3B} is methyl.

13. The compound according to claim 2, or a pharmaceutically acceptable salt thereof,
wherein
¹Y is CH and Y² is N;
X is a —S(O)₂CR⁶R⁷— linker group;
R¹ is a group selected from methyl, ethyl, propyl, butyl, isobutyl, tert-butyl, cyclopropyl, cyclopentyl cyclohexyl, phenyl, benzyl, phenethyl, imidazolyl, pyrrolidinyl, thiadiazolyl, thiazolyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, hydroxy, methyl, methoxy, trifluoromethyl, trifluoromethoxy, —NHCOCH₃, —CONH, and —CONHCH₃;
R¹ is 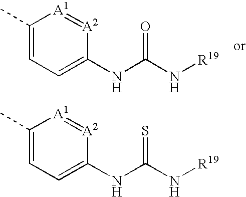
wherein A¹ and A² are selected from CH or N provided that at least one of A¹ or A² is CH;
R⁶ and R⁷ together with the carbon atom to which they are attached form a 3- to 5-membered carbocyclic ring; and
R¹⁹ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyrazolylmethyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C ,₆alkyl)carbamoyl.

14. The compound according to any one of claims 9 to 12, or a pharmaceutically acceptable salt thereof, where R⁶ and R⁷ together with the carbon atom to which they are attached form a cyclopropyl or cyclobutyl ring.

15. The compound of formula (I) according to claim 1 selected from any one of
1-[4-[4-(1-ethylsulfonylcyclopropyl)-6-[(3S)-3-methylmorpholin-4-yl]pyrimidin-2-yl]phenyl]-3-methyl-urea,
1-[4-[4-(1-cyclopropylsulfonylcyclopropyl)-6-[(3S)-3-methylmorpholin-4-yl]pyrimidin-2-yl]phenyl]-3-(2-hydroxyethyl)urea,
3-(2-hydroxyethyl)-1-[4-[4-[(3S)-3-methylmorpholin-4-yl]-6-(1-methylsulfonylcyclopropyl)pyrimidin-2-yl]phenyl]thiourea,
3-cyclopropyl-1-[4-[4-(1-methylsulfonylcyclopropyl)-6-morpholin-4-ylpyrimidin-2-yl]phenyl]urea,
1-[4-[4-[1-(4-chlorophenyl)sulfonylcyclopropyl]-6-[(3S)-3-methylmorpholin-4-yl]pyrimidn-2-yl]phenyl]-3-(2-hydroxyethyl)urea,
3-(2-hydroxyethyl)-1-[4-[4-[(3S)-3-methylmorpholin-4-yl]-6-(1-pyridin-2- ylsulfonylcyclopropyl)pyrimidin-2-yl]phenyl]urea,
3-(2-hydroxyethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
1-[4-[4-[1-(benzenesulfonyl)cyclopropyl]-6-[(3S)-3-methylmorpholin-4-yl]pyrimidin-2-yl]phenyl]-3-cyclopropylurea,
1-[4-[4-[1-(benzenesulfonyl)cyclopropyl]-6-[(3S)-3--methylmorpholin-4-yl]pyrimidin-2-yl]phenyl]-3-(2-hydroxyethyl)urea,
1-[4-[4-[1-(benzenesulfonyl)cyclopropyl]-6-[(3S)-3-methylmorpholin-4-yl]pyrimidin-2-yl]phenyl]-3-(3-hydroxypropyl)urea,
1-[4-[4-(1-cyclopropylsulfonylcyclopropyl)-6-[(3S)-3-methylmorpholin-4-yl]pyrimidin-2-yl]phenyl]-3-(2-hydroxyethyl)thiourea,
3-cyclopropyl-1-[4-[4-[1-(4-fluorophenyl)sulfonylcyclopropyl]-6-[(3S)-3-methylmorpholin-4-yl]pyrimidin-2-yl]phenyl]thiourea,
3-cyclopropyl-1-[4-[4-[1-(3-hydroxypropylsulfonyl)cyclopropyl]-6-[(3S)-3-methylmorpholin-4-yl]pyrimidin-2-yl]phenyl]thiourea,
1-ethyl-3-[4-[4-[1-(3-hydroxypropylsulfonyl)cyclopropyl]-6-[(3S)-3-methylmorpholin-4-yl]pyrimidin-2-yl]phenyl]thiourea,
1-[4-[4-(1-cyclopropylsulfonylcyclobutyl)-6-[(3S)-3-methylmorpholin-4-yl]pyrimidin-2-yl]phenyl]-3-(2-hydroxyethyl)urea,
1-[4-[4-[1-(benzenesulfonyl)cyclobutyl]-6-[(3S)-3-methylmorpholin-4-yl]pyrimidin-2-yl]phenyl]-3-(2-hydroxyethyl)urea,
1-[4-[4-[1-(benzenesulfonyl)cyclopropyl]-6-[(3S)-3-methylmorpholin-4-yl]pyrimidin-2-yl]phenyl]-3-methylthiourea,
1-[4-[4-[1-(benzenesulfonyl)cyclopropyl]-6-[(3S)-3-methylmorpholin-4-yl]pyrimidin-2-yl]phenyl]-3-(1H-imidazol-2-ylmethyl)thiourea,
3-(2-hydroxyethyl)-1-[4-[4-[(3S)-3-methylmorpholin-4-yl]-6-(1-pyridin-4-ylsulfonylcyclopropyl)pyrimidin-2-yl]phenyl]thiourea,
1-[4-[4-[1-(benzenesulfonyl)cyclopropyl]-6-[(3S)-3-methylmorpholin-4-yl]pyrimidin-2-yl]phenyl]-3-(2-fluoroethyl)urea,
1-[4-[4-[1-(2,4-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(3-fluorophenyl)sulfonylcyclopropyl]-6-[(3S)-3-methylmorpholin-4-yl]pyrimidin-2-yl]phenyl]-3-(2-hydroxyethyl)urea,
3-(2-hydroxyethyl)-1-[4-[4-[(3S)-3-methylmorpholin-4-yl]-6-[1-(2-methylphenyl)sulfonylcyclopropyl]pymidin-2-yl]phenyl]urea,
3-(2-fluoroethyl)-1-[4-[4-[(3S)-3-methylmorpholin-4-yl]-6-[1-(2-methylphenyl)sulfonylcyclopropyl]pyrimidin-2-yl]phenyl]urea,
3-(2-hydroxyethyl)-1-[4-[4-[(3S)-3-methylmorpholin-4-yl]-6-[1-(1,3-thiazol-2-ylsulfonyl)cyclopropyl]pyrimidin-2-yl]phenyl]urea,
1-[4-[4-[1-(2-chlorophenyl)sulfonylcyclopropyl]-6-[(3S)-3-methylmorpholin-4-yl]pyrimidin-2-yl]phenyl]-3-(2-hydroxyethyl)urea,
1-[4-[4-[1-(benzenesulfonyl)cyclobutyl]-6-[(3S)-3-methylmorpholin-4-yl]pyrimidin-2-yl]phenyl]-3-[(2S)-1-hydroxypropan-2-yl]urea,
1-[4-[4-(1-cyclopropylsulfonylcyclobutyl)-6-[(3S)-3-methylmorpholin-4-yl]pyrimidin-2-yl]phenyl]-3-[(2S)-1-hydroxypropan-2-yl]urea,
1-[4-[4-[1-(benzenesulfonyl)cyclobutyl]-6-[(3S)-3-methylmorpholin-4-yl]pyrimidin-2-yl]phenyl]-3-[(2R)-1-hydroxypropan-2-yl]urea,
3-(2-fluoroethyl)-1-[4-[4-[(3S)-3-methylmorpholin-4-yl]-6-[1-[(4-methyl-1,3-thiazol-2-yl)sulfonyl]cyclopropyl]pyrimidin-2-yl]phenyl]urea,
3-(2-hydroxyethyl)-1-[4-[4-[(3S)-3-methylmorpholin-4-yl]-6-[1-[(4-methyl-1,3-thiazol-2-yl)sulfonyl]cyclobutyl]pyrimidin-2-yl]phenyl]urea,
3-(2-hydroxyethyl)-1-[4-[4-[(3S)-3-methylmorpholin-4-yl]-6-[1-(1,3-thiazol-2-ylsulfonyl)cyclobutyl]pyrimidin-2-yl]phenyl]urea,
1-[4-[4-[1-(benzenesulfonyl)cyclopropyl]-6-[(3S)-3-ethylmorpholin-4-yl]pyrimidin-2-ylvphenyl]-3-methylurea,
1-[4-[4-[1-(benzenesulfonyl)cyclopropyl]-6-[(3S)-3-ethylmorpholin-4-yl]pyrimidin-2-yl]phenyl]-3-cyclopropylurea,
1-[4-[4-[1-(benzenesulfonyl)cyclopropyl]-6-[(3S)-3-ethylmorpholin-4-yl]pyrimidin-2-yl]phenyl]-3-(2-hydroxyethyl)urea,
1-[4-[4-[1-(benzenesulfonyl)cyclopropyl]-6-[(3S)-3-ethylmorpholin-4-yl]pyrimidin-2-yl]phenyl]-3-(2-fluoroethyl)urea,
1-[4-[4-[1-(benzenesulfonyl)cyclopropyl]-6-[(3S)-3-methylmorpholin-4-yl]pyrimidin-2-yl]phenyl]-3-(2,2-difluoroethyl)urea,
1-[4-[4-[1-(benzenesulfonyl)cyclopropyl]-6-[(3S)-3-metbylmorpholin-4-yl]pyrimidin-2-yl]phenyl]-3-[(2S)-1-hydroxypropan-2-yl]urea,
1-[4-[4-[1-(benzenesulfonyl)cyclopropyl]-6-[(3S)-3-methylmorpholin-4-yl]pyrimidin-2-yl]phenyl]-3-[(2R)-1-hydroxypropan-2-yl]urea,
3-cyclopropyl-1-[4-[4-[(3S)-3-ethylmorpholin-4-yl]-6-(1-methylsulfonylcyclopropyl)pyrimidin-2-yl]phenyl]urea,
1-[4-[4-[(3S)-3-ethylmorpholin-4-yl]-6-(1-methylsulfonylcyclopropyl)pyrimidin-2-yl]phenyl]-3-(2-hydroxyethyl)urea,
1-[4-[4-[1-(2,6-difluorophenyl)sulfonylcyclopropyl]-6-[(3S)-3-methylmorpholin-4-yl]pyrimidin-2-yl]phenyl]-3-(2-hydroxyethyl)urea,
1-[4-[4-(1-cyclopropylsulfonylcyclopropyl)-6-[(3S,5S)-3,5-dimethylmorpholin-4-yl]pyrimidin-2-yl]phenyl]-3-(2-hydroxyethyl)urea,
3-cyclopropyl-1-[4-[4-[(3S,5S)-3,5-dimethylmorpholin-4-yl]-6-(1-methylsulfonylcyclopropyl)pyrimidin-2-yl]phenyl]urea,
1-[4-[4-[(3S,5S)-3,5-dimethylmorpholin-4-yl]-6-(1-methylsulfonylcyclopropyl)pyrimidin-2-yl]phenyl]-3-(2-fluoroethyl)urea,
1-[4-[4-[(3S,5S)-3,5-dimethylmorpholin-4-yl]-6-(1-methylsulfonylcyclopropyl)pyrimidin-2-yl]phenyl]-3-(2-hydroxyethyl)urea,
1-[4-[4-[1-(benzenesulfonyl)cyclopropyl]-6-[(3S,5S)-3,5-dimethylmorpholin-4-yl]pyrimidin-2-yl]phenyl]-3-methylurea,
1-[4-[4-[1-(benzenesulfonyl)cyclopropyl]-6-[(3S,5S)-3,5-dimethylmorpholin-4-yl]pyrimidin-2-yl]phenyl]-3-cyclopropylurea,
1-[4-[4-[1-(benzenesulfonyl)cyclopropyl]-6-[(3S,5S)-3,5-dimethylmorpholin-4-yl]pyrimidin-2-yl]phenyl]-3-(2-hydroxyethyl)urea,
1-[4-[4-[1-(benzenesulfonyl)cyclopropyl]-6-[(3S)-3-ethylmorpholin-4-yl]pyrimidin-2-yl]phenyl]-3-[(2S)-1-hydroxypropan-2-yl]urea,
1-[4-[4-[1-(benzenesulfonyl)cyclopropyl]-6-[(3S)-3-ethylmorpholin-4-yl]pyrimidin-2-yl]phenyl]-3-(3-hydroxypropyl)urea,
1-[4-[4-[1-(benzenesulfonyl)cyclopropyl]-6-[(3S)-3-ethylmorpholin-4-yl]pyrimidin-2-yl]phenyl]-3-[(1-hydroxycyclopropyl)methyl]urea,
1-[4-[4-(1-ethylsulfonylcyclobutyl)-6-[(3S)-3-methylmorpholin-4-yl]pyrimidin-2-yl]phenyl]-3-(2-hydroxyethyl)thiourea,
1-[4-[4-[(3S,5S)-3,5-dimethylmorpholin-4-yl]-6-(1-methylsulfonylcyclopropyl)pyrimidin-2-yl]phenyl]-3-ethylurea,
3-[(2S)-1-hydroxypropan-2-yl]-1-[4-[4-[(3S)-3-methylmorpholin-4-yl]-6-[1-(1,3-thiazol-2-ylsulfonyl)cyclobutyl]pyrimidin-2-yl]phenyl]urea, and
1-[4-[4-[1-(benzenesulfonyl)cyclopropyl]-6-[(3S,5S)-3,5-dimethylmorpholin-4-yl]pyrimidin-2-yl]phenyl]-3-(2-fluoroethyl)urea,
or a pharmaceutically acceptable salt thereof.

16. (canceled)

17. A method for producing an anti-proliferative effect in a warm-blooded animal in need of such treatment which comprises administering to said animal an effective amount of a compound of formula (I) according to claim 1, or a pharmaceutically acceptable salt thereof.

18. A method for producing a mTOR kinase inhibitory effect in a warm-blooded animal in need of such treatment which comprises administering to said animal an effective amount of a compound of formula (I) according to claim 1, or a pharmaceutically acceptable salt thereof.

19. A method for treating cancer, inflammatory diseases, obstructive airways diseases, immune diseases or cardiovascular diseases in a warm blooded animal that is in need of such treatment which comprises administering to said animal an effective amount of a compound of formula (I) according to claim 1, or a pharmaceutically acceptable salt thereof.

20. A pharmaceutical composition comprising a compound of formula (I) according to claim 1, or a pharmaceutically acceptable salt thereof, in association with a pharmaceutically acceptable diluent or carrier.
